(19) United States
(12) Patent Application Publication
(10) Pub. No.: US 2007/0185017 A1
(43) Pub. Date: Aug. 9, 2007

(54) Compositions and methods for the treatment of immune related diseases

(75) Inventors: Sudeepta Aggarwal, North Potomac, MD (US); Hilary Clark, San Francisco, CA (US); Austin L. Gurney, San Francisco, CA (US); Jill R. Schoenfeld, Ashland, OR (US); P. Mickey Williams, Half Moon Bay, CA (US); William I. Wood, Cupertino, CA (US); Thomas D. Wu, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 10/533,069

(22) PCT Filed: Oct. 28, 2003
(86) PCT No.: PCT/US03/34381

§ 371 Date: Jan. 3, 2007

Related U.S. Application Data
(60) Provisional application No. 60/422,472, filed on Oct. 29, 2002.

(51) Int. Cl. A61K 38/17; C07K 14/47; C07K 16/18; C12P 21/06; C12N 9/00

(52) U.S. Cl. 514/12; 530/350; 530/388.1; 435/69.1; 435/183; 435/320.1; 435/325

(57) ABSTRACT

The present invention relates to compositions containing novel proteins and methods of using those compositions for the diagnosis and treatment of immune related diseases.

### FIELD OF THE INVENTION

The present invention relates to compositions and methods useful for the diagnosis and treatment of immune related diseases.

### BACKGROUND OF THE INVENTION

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host mammal. T cells include helper T cells and cytotoxic T cells. Helper T cells proliferate extensively following recognition of an antigen-MHC complex on an antigen presenting cell. Helper T cells also secrete a variety of cytokines, i.e., lymphokines, which play a central role in the activation of B cells, cytotoxic T cells and a variety of other cells which participate in the immune response.

Immune related diseases could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

T cells play a central role in host defense. T cells are able to modulate the immune response of other cell lineages through the production of a variety of cytokines and immune modulatory molecules. In addition they are responsible for surveying cells throughout the organism for the presence of non-self. This highly sophisticated process utilizes the T cell receptor (TCR), which is able to recognize and discriminate between self and non-self peptides displayed by the MHC complex on other cells. This process also integrates co-stimulatory signals that provide additional information to the T cell about the nature of the potential non-self threat. These two signals, the TCR signal and the co-stimulatory signal can be experimentally triggered by use of agonist antibodies such as certain antibodies to the T cell receptor component CD3, and the co-stimulatory receptor CD28. While T cells are essential components of normal immune function, it is believed that inappropriate T cell function underlies many very serious medical conditions including autoimmune disease. Diseases that are impacted by pathologic T cell function are thought to include asthma, arthritis, psoriasis, multiple sclerosis, inflammatory bowel disease, diabetes, graft versus host disease and many others. In these diseases the portion of the T cell repertoire that has a “memory” phenotype is thought to contribute to the disease pathology. It is therefore of great importance to understand the molecular events that occur upon activation of memory T cells. In humans, memory T cells can be identified through the use of the antigen CD45RO which is expressed on memory T cells but not on resting naïve T cells. The use of DNA microarrays provides a powerful experimental approach to identify molecular changes that occur upon activation of this critical cell population. Understanding the identity of molecules whose expression is altered upon memory T cell activation can enable therapeutic strategies that target the pathways impacted by these alterations in gene expression. Such therapeutic strategies can include the use of recombinant proteins, soluble receptors, antibodies, peptides, or small molecule drugs.

Despite the above identified advances in T cell research, there is a great need for additional diagnostic and therapeutic agents capable of detecting the presence of memory T cell mediated disorders in a mammal and for effectively reducing these disorders. Accordingly, it is an objective of the present invention to identify polypeptides that are overexpressed in memory T cells as compared to non-memory T cells, and to use those polypeptides, and their encoding nucleic acids, to produce compositions of matter useful in the therapeutic treatment and diagnostic detection of memory T cell mediated disorders in mammals.

### SUMMARY OF THE INVENTION

### A. Embodiments

The present invention concerns compositions and methods useful for the diagnosis and treatment of immune related disease in mammals, including humans. The present invention is based on the identification of proteins (including agonist and antagonist antibodies) which are a result of stimulation of the immune response in mammals. Immune related diseases can be treated by suppressing or enhancing the immune response. Molecules that enhance the immune response stimulate or potentiate the immune response to an antigen. Molecules which stimulate the immune response can be used therapeutically where enhancement of the immune response would be beneficial. Alternatively, molecules that suppress the immune response attenuate or reduce the immune response to an antigen (e.g., neutralizing antibodies) can be used therapeutically where attenuation of the immune response would be beneficial (e.g., inflammation). Accordingly, the PRO polypeptides, agonists and antagonists thereof are also useful to prepare medicines and medicaments for the treatment of immune-related and inflammatory diseases. In a specific aspect, such medicines and medicaments comprise a therapeutically effective amount of a PRO polypeptide, agonist or antagonist thereof with a pharmaceutically acceptable carrier. Preferably, the admixture is sterile.

In a further embodiment, the invention concerns a method of identifying agonists or antagonists to a PRO polypeptide which comprises contacting the PRO polypeptide with a candidate molecule and monitoring a biological activity mediated by said PRO polypeptide. Preferably, the PRO polypeptide is a native sequence PRO polypeptide. In a specific aspect, the PRO agonist or antagonist is an anti-PRO antibody.

In another embodiment, the invention concerns a composition of matter comprising a PRO polypeptide or an agonist or antagonist antibody which binds the polypeptide in admixture with a carrier or excipient. In one aspect, the composition comprises a therapeutically effective amount of the polypeptide or antibody. In another aspect, when the composition comprises an immune stimulating molecule, the composition is useful for: (a) increasing infiltration of inflammatory cells into a tissue of a mammal in need thereof, (b) stimulating or enhancing an immune response in a mammal in need thereof, (c) increasing the proliferation of T-lymphocytes in a mammal in need thereof in response to an antigen, (d) stimulating the activity of T-lymphocytes or (e) increasing the vascular permeability. In a further aspect, when the composition comprises an immune inhibiting molecule, the composition is useful for: (a) decreasing infiltration of inflammatory cells into a tissue of a mammal in need thereof, (b) inhibiting or reducing an immune response in a mammal in need thereof, (c) decreasing the activity of T-lymphocytes or (d) decreasing the proliferation of T-lymphocytes in a mammal in need thereof in response to an antigen. In another aspect, the composition comprises a further active ingredient, which may, for example, be a further antibody or a cytotoxic or chemotherapeutic agent. Preferably, the composition is sterile.

In another embodiment, the invention concerns a method of treating an immune related disorder in a mammal in need thereof, comprising administering to the mammal an effective amount of a PRO polypeptide, an agonist thereof, or an antagonist thereto. In a preferred aspect, the immune related disorder is selected from the group consisting of: systemic lupus erythematosis, rheumatoid arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In another embodiment, the invention provides an antibody which specifically binds to any of the above or below described polypeptides. Optionally, the antibody is a monoclonal antibody, humanized antibody, antibody fragment or single-chain antibody. In one aspect, the present invention concerns an isolated antibody which binds a PRO polypeptide. In another aspect, the antibody mimics the activity of a PRO polypeptide (an agonist antibody) or conversely the antibody inhibits or neutralizes the activity of a PRO polypeptide (an antagonist antibody). In another aspect, the antibody is a monoclonal antibody, which preferably has nonhuman complementarity determining region (CDR) residues and human framework region (FR) residues. The antibody may be labeled and may be immobilized on a solid support. In a further aspect, the antibody is an antibody fragment, a monoclonal antibody, a single-chain antibody, or an anti-idiotypic antibody.

In yet another embodiment, the present invention provides a composition comprising an anti-PRO antibody in admixture with a pharmaceutically acceptable carrier. In one aspect, the composition comprises a therapeutically effective amount of the antibody. Preferably, the composition is sterile. The composition may be administered in the form of a liquid pharmaceutical formulation, which may be preserved to achieve extended storage stability. Alternatively, the antibody is a monoclonal antibody, an antibody fragment, a humanized antibody, or a single-chain antibody.

In a further embodiment, the invention concerns an article of manufacture, comprising:

(a) a composition of matter comprising a PRO polypeptide or agonist or antagonist thereof;

(b) a container containing said composition; and

(c) a label affixed to said container, or a package insert included in said container referring to the use of said PRO polypeptide or agonist or antagonist thereof in the treatment of an immune related disease. The composition may comprise a therapeutically effective amount of the PRO polypeptide or the agonist or antagonist thereof.

In yet another embodiment, the present invention concerns a method of diagnosing an immune related disease in a mammal, comprising detecting the level of expression of a gene encoding a PRO polypeptide (a) in a test sample of tissue cells obtained from the mammal, and (b) in a control sample of known normal tissue cells of the same cell type, wherein a higher or lower expression level in the test sample as compared to the control sample indicates the presence of immune related disease in the mammal from which the test tissue cells were obtained.

In another embodiment, the present invention concerns a method of diagnosing an immune disease in a mammal, comprising (a) contacting an anti-PRO antibody with a test sample of tissue cells obtained from the mammal, and (b) detecting the formation of a complex between the antibody and a PRO polypeptide, in the test sample; wherein the formation of said complex is indicative of the presence or absence of said disease. The detection may be qualitative or quantitative, and may be performed in comparison with monitoring the complex formation in a control sample of known normal tissue cells of the same cell type. A larger quantity of complexes formed in the test sample indicates the presence or absence of an immune disease in the mammal from which the test tissue cells were obtained. The antibody preferably carries a detectable label. Complex formation can be monitored, for example, by light microscopy, flow cytometry, fluorimetry, or other techniques known in the art. The test sample is usually obtained from an individual suspected of having a deficiency or abnormality of the immune system.

In another embodiment, the invention provides a method for determining the presence of a PRO polypeptide in a sample comprising exposing a test sample of cells suspected of containing the PRO polypeptide to an anti-PRO antibody and determining the binding of said antibody to said cell sample. In a specific aspect, the sample comprises a cell suspected of containing the PRO polypeptide and the antibody binds to the cell. The antibody is preferably detectably labeled and/or bound to a solid support.

In another embodiment, the present invention concerns an immune-related disease diagnostic kit, comprising an anti-PRO antibody and a carrier in suitable packaging. The kit preferably contains instructions for using the antibody to detect the presence of the PRO polypeptide. Preferably the carrier is pharmaceutically acceptable.

In another embodiment, the present invention concerns a diagnostic kit, containing an anti-PRO antibody in suitable packaging. The kit preferably contains instructions for using the antibody to detect the PRO polypeptide.

In another embodiment, the invention provides a method of diagnosing an immune-related disease in a mammal which comprises detecting the presence or absence or a PRO polypeptide in a test sample of tissue cells obtained from said mammal, wherein the presence or absence of the PRO polypeptide in said test sample is indicative of the presence of an immune-related disease in said mammal.

In another embodiment, the present invention concerns a method for identifying an agonist of a PRO polypeptide comprising:

(a) contacting cells and a test compound to be screened under conditions suitable for the induction of a cellular response normally induced by a PRO polypeptide; and

(b) determining the induction of said cellular response to determine if the test compound is an effective agonist, wherein the induction of said cellular response is indicative of said test compound being an effective agonist.

In another embodiment, the invention concerns a method for identifying a compound capable of inhibiting the activity of a PRO polypeptide comprising contacting a candidate compound with a PRO polypeptide under conditions and for a time sufficient to allow these two components to interact and determining whether the activity of the PRO polypeptide is inhibited. In a specific aspect, either the candidate compound or the PRO polypeptide is immobilized on a solid support. In another aspect, the non-immobilized component carries a detectable label. In a preferred aspect, this method comprises the steps of:

(a) contacting cells and a test compound to be screened in the presence of a PRO polypeptide under conditions suitable for the induction of a cellular response normally induced by a PRO polypeptide; and

(b) determining the induction of said cellular response to determine if the test compound is an effective antagonist.

In another embodiment, the invention provides a method for identifying a compound that inhibits the expression of a PRO polypeptide in cells that normally express the polypeptide, wherein the method comprises contacting the cells with a test compound and determining whether the expression of the PRO polypeptide is inhibited. In a preferred aspect, this method comprises the steps of:

(a) contacting cells and a test compound to be screened under conditions suitable for allowing expression of the PRO polypeptide; and

(b) determining the inhibition of expression of said polypeptide.

In yet another embodiment, the present invention concerns a method for treating an immune-related disorder in a mammal that suffers therefrom comprising administering to the mammal a nucleic acid molecule that codes for either (a) a PRO polypeptide, (b) an agonist of a PRO polypeptide or (c) an antagonist of a PRO polypeptide, wherein said agonist or antagonist may be an anti-PRO antibody. In a preferred embodiment, the mammal is human. In another preferred embodiment, the nucleic acid is administered via ex vivo gene therapy. In a further preferred embodiment, the nucleic acid is comprised within a vector, more preferably an adenoviral, adeno-associated viral, lentiviral or retroviral vector.

In yet another aspect, the invention provides a recombinant viral particle comprising a viral vector consisting essentially of a promoter, nucleic acid encoding (a) a PRO polypeptide, (b) an agonist polypeptide of a PRO polypeptide, or (c) an antagonist polypeptide of a PRO polypeptide, and a signal sequence for cellular secretion of the polypeptide, wherein the viral vector is in association with viral structural proteins. Preferably, the signal sequence is from a mammal, such as from a native PRO polypeptide.

In a still further embodiment, the invention concerns an ex vivo producer cell comprising a nucleic [symbol] construct that expresses retroviral structural proteins and also comprises a retroviral vector consisting [symbol] ually of a promoter, nucleic acid encoding (a) a PRO polypeptide, (b) an agonist polypeptide of a PRO [symbol] peptide or (c) an antagonist polypeptide of a PRO polypeptide, and a signal sequence for cellular [symbol] tion of the polypeptide, wherein said producer cell packages the retroviral vector in association with the [symbol] ural proteins to produce recombinant retroviral particles.

In a still further embodiment, the invention provides a method of increasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is increased.

In a still further embodiment, the invention provides a method of decreasing the activity of T-lymphocytes in a mammal comprising administering to said mammal (a) a PRO polypeptide, (b) an agonist of a PRO polypeptide, or (c) an antagonist of a PRO polypeptide, wherein the activity of T-lymphocytes in the mammal is decreased.

In a still further embodiment, the invention provides a method of increasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is increased.

In a still further embodiment, the invention provides a method of decreasing the proliferation of T-lymphocytes in a mammal comprising administering to said mammal (a) a PRO polypeptide, (b) an agonist of a PRO polypeptide, or (c) an antagonist of a PRO polypeptide, wherein the proliferation of T-lymphocytes in the mammal is decreased.

### B. Additional Embodiments

In other embodiments of the present invention, the invention provides vectors comprising DNA encoding any of the herein described polypeptides. Host cell comprising any such vector are also provided. By way of example, the host cells may be CHO cells, E. coli, or yeast. A process for producing any of the herein described polypeptides is further provided and comprises culturing host cells under conditions suitable for expression of the desired polypeptide and recovering the desired polypeptide from the cell culture.

In other embodiments, the invention provides chimeric molecules comprising any of the herein described polypeptides fused to a heterologous polypeptide or amino acid sequence. Example of such chimeric molecules comprise any of the herein described polypeptides fused to an epitope tag sequence or a Fc region of an immunoglobulin.

In another embodiment, the invention provides an antibody which specifically binds to any of the above or below described polypeptides. Optionally, the antibody is a monoclonal antibody, humanized antibody, antibody fragment or single-chain antibody.

In yet other embodiments, the invention provides oligonucleotide probes useful for isolating genomic and cDNA nucleotide sequences or as antisense probes, wherein those probes may be derived from any of the above or below described nucleotide sequences.

In other embodiments, the invention provides an isolated nucleic acid molecule comprising a nucleotide sequence that encodes a PRO polypeptide.

In one aspect, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule encoding a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein, or (b) the complement of the DNA molecule of (a).

In other aspects, the isolated nucleic acid molecule comprises a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule comprising the coding sequence of a full-length PRO polypeptide cDNA as disclosed herein, the coding sequence of a PRO polypeptide lacking the signal peptide as disclosed herein, the coding sequence of an extracellular domain of a transmembrane PRO polypeptide, with or without the signal peptide, as disclosed herein or the coding sequence of any other specifically defined fragment of the full-length amino acid sequence as disclosed herein, or (b) the complement of the DNA molecule of (a).

In a further aspect, the invention concerns an isolated nucleic acid molecule comprising a nucleotide sequence having at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity to (a) a DNA molecule that encodes the same mature polypeptide encoded by any of the human protein cDNAs as disclosed herein, or (b) the complement of the DNA molecule of (a).

Another aspect the invention provides an isolated nucleic acid molecule comprising a nucleotide sequence encoding a PRO polypeptide which is either transmembrane domain-deleted or transmembrane domain-inactivated, or is complementary to such encoding nucleotide sequence, wherein the transmembrane domain(s) of such polypeptide are disclosed herein. Therefore, soluble extracellular domains of the herein described PRO polypeptides are contemplated.

Another embodiment is directed to fragments of a PRO polypeptide coding sequence, or the complement thereof, that may find use as, for example, hybridization probes, for encoding fragments of a PRO polypeptide that may optionally encode a polypeptide comprising a binding site for an anti-PRO antibody or as antisense oligonucleotide probes. Such nucleic acid fragments are usually at least about 20 nucleotides in length, alternatively at least about 30 nucleotides in length, alternatively at least about 40 nucleotides in length, alternatively at least about 50 nucleotides in length, alternatively at least about 60 nucleotides in length, alternatively at least about 70 nucleotides in length, alternatively at least about 80 nucleotides in length, alternatively at least about 90 nucleotides in length, alternatively at least about 100 nucleotides in length, alternatively at least about 110 nucleotides in length, alternatively at least about 120 nucleotides in length, alternatively at least about 130 nucleotides in length, alternatively at least about 140 nucleotides in length, alternatively at least about 150 nucleotides in length, alternatively at least about 160 nucleotides in length, alternatively at least about 170 nucleotides in length, alternatively at least about 180 nucleotides in length, alternatively at least about 190 nucleotides in length, alternatively at least about 200 nucleotides in length, alternatively at least about 250 nucleotides in length, alternatively at least about 300 nucleotides in length, alternatively at least about 350 nucleotides in length, alternatively at least about 400 nucleotides in length, alternatively at least about 450 nucleotides in length, alternatively at least about 500 nucleotides in length, alternatively at least about 600 nucleotides in length, alternatively at least about 700 nucleotides in length, alternatively at least about 800 nucleotides in length, alternatively at least about 900 nucleotides in length and alternatively at least about 1000 nucleotides in length, wherein in this context the term “about” means the referenced nucleotide sequence length plus or minus 10% of that referenced length. It is noted that novel fragments of a PRO polypeptide-encoding nucleotide sequence may be determined in a routine manner by aligning the PRO polypeptide-encoding nucleotide sequence with other known nucleotide sequences using any of a number of well known sequence alignment programs and determining which PRO polypeptide-encoding nucleotide sequence fragment(s) are novel. All of such PRO polypeptide-encoding nucleotide sequences are contemplated herein. Also contemplated are the PRO polypeptide fragments encoded by these nucleotide molecule fragments, preferably those PRO polypeptide fragments that comprise a binding site for an anti-PRO antibody.

In another embodiment, the invention provides isolated PRO polypeptide encoded by any of the isolated nucleic acid sequences herein above identified.

In a certain aspect, the invention concerns an isolated PRO polypeptide, comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a PRO polypeptide having a full-length amino acid sequence as disclosed herein, an amino acid sequence lacking the signal peptide as disclosed herein, an extracellular domain of a transmembrane protein, with or without the signal peptide, as disclosed herein or any other specifically defined fragment of the full-length amino acid sequence as disclosed herein.

In a further aspect, the invention concerns an isolated PRO polypeptide comprising an amino acid sequence having at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to an amino acid sequence encoded by any of the human protein cDNAs as disclosed herein.

In a specific aspect, the invention provides an isolated PRO polypeptide without the N-terminal signal sequence and/or the initiating methionine and is encoded by a nucleotide sequence that encodes such an amino acid sequence as herein before described. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

Another aspect the invention provides an isolated PRO polypeptide which is either transmembrane domain-deleted or transmembrane domain-inactivated. Processes for producing the same are also herein described, wherein those processes comprise culturing a host cell comprising a vector which comprises the appropriate encoding nucleic acid molecule under conditions suitable for expression of the PRO polypeptide and recovering the PRO polypeptide from the cell culture.

In yet another embodiment, the invention concerns agonists and antagonists of a native PRO polypeptide as defined herein. In a particular embodiment, the agonist or antagonist is an anti-PRO antibody or a small molecule.

In a further embodiment, the invention concerns a method of identifying agonists or antagonists to a PRO polypeptide which comprise contacting the PRO polypeptide with a candidate molecule and monitoring a biological activity mediated by said PRO polypeptide. Preferably, the PRO polypeptide is a native PRO polypeptide.

In a still further embodiment, the invention concerns a composition of matter comprising a PRO polypeptide, or an agonist or antagonist of a PRO polypeptide as herein described, or an anti-PRO antibody, in combination with a carrier. Optionally, the carrier is a pharmaceutically acceptable carrier.

Another embodiment of the present invention is directed to the use of a PRO polypeptide, or an agonist or antagonist thereof as herein before described, or an anti-PRO antibody, for the preparation of a medicament useful in the treatment of a condition which is responsive to the PRO polypeptide, an agonist or antagonist thereof or an anti-PRO antibody.

### BRIEF DESCRIPTION OF THE DRAWINGS

The FIGS. 1-2442 show the nucleic acids of the invention and their encoded PRO polypeptides.

FIG. 1 shows a nucleotide sequence (SEQ ID NO:1) of a native sequence PRO84739 cDNA, wherein SEQ ID NO:1 is a clone designated herein as “DNA329084”.

FIG. 2 shows the amino acid sequence (SEQ ID NO:2) derived from the coding sequence of SEQ ID NO:1 shown in FIG. 1.

FIG. 3 shows a nucleotide sequence (SEQ ID NO:3) of a native sequence PRO61679 cDNA, wherein SEQ ID NO3: is a clone designated herein as “DNA273712”.

FIG. 4 shows the amino acid sequence (SEQ ID NO:4) derived from the coding sequence of SEQ ID NO:3 shown in FIG. 3.

FIG. 5 shows a nucleotide sequence (SEQ ID NO:5) of a native sequence PRO83580 cDNA, wherein SEQ ID NO5: is a clone designated herein as “DNA327535”.

FIG. 6 shows the amino acid sequence (SEQ ID NO:6) derived from the coding sequence of SEQ ID NO5: shown in FIG. 5.

FIG. 7 shows a nucleotide sequence (SEQ ID NO:7) of a native sequence PRO60800 cDNA, wherein SEQ ID NO: is a clone designated herein as “DNA326466”.

FIG. 8 shows the amino acid sequence (SEQ ID NO:8) derived from the coding sequence of SEQ ID NO: 7 shown in FIG. 7.

FIG. 9 shows a nucleotide sequence (SEQ ID NO:9) of a native sequence PRO84740 cDNA, wherein SEQ ID NO:9 is a clone designated herein as “DNA329085”.

FIG. 10 shows the amino acid sequence (SEQ ID NO:10) derived from the coding sequence of SEQ ID NO:9 shown in FIG. 9.

FIG. 11 shows a nucleotide sequence (SEQ ID NO:11) of a native sequence PRO84741 cDNA, wherein SEQ ID NO:11 is a clone designated herein as “DNA329086”.

FIG. 12 shows the amino acid sequence (SEQ ID NO:12) derived from the coding sequence of SEQ ID NO:11 shown in FIG. 11.

FIG. 13 shows a nucleotide sequence (SEQ ID NO:13) of a native sequence PRO69614 cDNA, wherein SEQ ID NO:13 is a clone designated herein as “DNA329087”.

FIG. 14 shows the amino acid sequence (SEQ ID NO:14) derived from the coding sequence of SEQ ID NO:13 shown in FIG. 13.

FIG. 15 shows a nucleotide sequence (SEQ ID NO:15) of a native sequence PRO71125 cDNA, wherein SEQ ID NO:15 is a clone designated herein as “DNA324783”.

FIG. 16 shows the amino acid sequence (SEQ ID NO:16) derived from the coding sequence of SEQ ID NO:15 shown in FIG. 15.

FIG. 17 shows a nucleotide sequence (SEQ ID NO:17) of a native sequence PRO40279 cDNA, wherein SEQ ID NO:17 is a clone designated herein as “DNA329088”.

FIG. 18 shows the amino acid sequence (SEQ ID NO:18) derived from the coding sequence of SEQ ID NO:17 shown in FIG. 17.

FIG. 19 shows a nucleotide sequence (SEQ ID NO:19) of a native sequence PRO60747 cDNA, wherein SEQ ID NO:19 is a clone designated herein as “DNA272614”.

FIG. 20 shows the amino acid sequence (SEQ ID NO:20) derived from the coding sequence of SEQ ID NO:19 shown in FIG. 19.

FIG. 21 shows a nucleotide sequence (SEQ ID NO:21) of a native sequence PRO71106 cDNA, wherein SEQ ID NO:21 is a clone designated herein as “DNA304680”.

FIG. 22 shows the amino acid sequence (SEQ ID NO:22) derived from the coding sequence of SEQ ID NO:21 shown in FIG. 21.

FIG. 23 shows a nucleotide sequence (SEQ ID NO:23) of a native sequence PRO37034 cDNA, wherein SEQ ID NO:23 is a clone designated herein as “DNA226571”.

FIG. 24 shows the amino acid sequence (SEQ ID NO:24) derived from the coding sequence of SEQ ID NO:23 shown in FIG. 23.

FIG. 25 shows a nucleotide sequence (SEQ ID NO:25) of a native sequence PRO84742 cDNA, % herein SEQ ID NO:25 is a clone designated herein as “DNA329089”.

FIG. 26 shows the amino acid sequence (SEQ ID NO:26) derived from the coding sequence of SEQ ID NO:25 shown in FIG. 25.

FIG. 27 shows a nucleotide sequence (SEQ ID NO:27) of a native sequence PRO84743 cDNA, wherein SEQ ID NO:27 is a clone designated herein as “DNA329090”.

FIG. 28 shows the amino acid sequence (SEQ ID NO:28) derived from the coding sequence of SEQ ID NO:27 shown in FIG. 27.

FIG. 29 shows a nucleotide sequence (SEQ ID NO:29) of a native sequence PRO22637 cDNA, wherein SEQ ID NO:29 is a clone designated herein as “DNA189703”.

FIG. 30 shows the amino acid sequence (SEQ ID NO:30) derived from the coding sequence of SEQ ID NO:29 shown in FIG. 29.

FIG. 31 shows a nucleotide sequence (SEQ ID NO:31) of a native sequence PRO81962 cDNA, wherein SEQ ID NO:31 is a clone designated herein as “DNA325438”.

FIG. 32 shows the amino acid sequence (SEQ ID NO:32) derived from the coding sequence of SEQ ID NO:31 shown in FIG. 31.

FIG. 33 shows a nucleotide sequence (SEQ ID NO:33) of a native sequence PRO11997 cDNA, wherein SEQ ID NO:33 is a clone designated herein as “DNA329091”.

FIG. 34 shows the amino acid sequence (SEQ ID NO:34) derived from the coding sequence of SEQ ID NO:33 shown in FIG. 33.

FIG. 35 shows a nucleotide sequence (SEQ ID NO:35) of a native sequence PRO59293 cDNA, wherein SEQ ID NO:35 is a clone designated herein as “DNA270963”.

FIG. 36 shows the amino acid sequence (SEQ ID NO:36) derived from the coding sequence of SEQ ID NO:35 shown in FIG. 35.

FIG. 37 shows a nucleotide sequence (SEQ ID NO:37) of a native sequence PRO33667 cDNA, wherein SEQ ID NO:37 is a clone designated herein as “DNA210121”.

FIG. 38 shows the amino acid sequence (SEQ ID NO:38) derived from the coding sequence of SEQ ID NO:37 shown in FIG. 37.

FIG. 39 shows a nucleotide sequence (SEQ ID NO:39) of a native sequence PRO84744 cDNA, wherein SEQ ID NO:39 is a clone designated herein as “DNA329092”.

FIG. 40 shows the amino acid sequence (SEQ ID NO:40) derived from the coding sequence of SEQ ID NO:39 shown in FIG. 39.

FIG. 41 shows a nucleotide sequence (SEQ ID NO:41) of a native sequence PRO49242 cDNA, wherein SEQ ID NO:41 is a clone designated herein as “DNA254127”.

FIG. 42 shows the amino acid sequence (SEQ ID NO:42) derived from the coding sequence of SEQ ID NO:41 shown in FIG. 41.

FIG. 43 shows a nucleotide sequence (SEQ ID NO:43) of a native sequence PRO4546 cDNA, wherein SEQ ID NO:43 is a clone designated herein as “DNA103216”.

FIG. 44 shows the amino acid sequence (SEQ ID NO:44) derived from the coding sequence of SEQ ID NO:43 shown in FIG. 43.

FIG. 45 shows a nucleotide sequence (SEQ ID NO:45) of a native sequence PRO38028 cDNA, wherein SEQ ID NO:45 is a clone designated herein as “DNA328356”.

FIG. 46 shows the amino acid sequence (SEQ ID NO:46) derived from the coding sequence of SEQ ID NO:45 shown in FIG. 45.

FIG. 47 shows a nucleotide sequence (SEQ ID NO:47) of a native sequence PRO65 cDNA, wherein SEQ ID NO:47 is a clone designated herein as “DNA324158”.

FIG. 48 shows the amino acid sequence (SEQ ID NO:48) derived from the coding sequence of SEQ ID NO:47 shown in FIG. 47.

FIG. 49 shows a nucleotide sequence (SEQ ID NO:49) of a native sequence PRO84745 cDNA, wherein SEQ ID NO:49 is a clone designated herein as “DNA329093”.

FIG. 50 shows the amino acid sequence (SEQ ID NO:50) derived from the coding sequence of SEQ ID NO:49 shown in FIG. 49.

FIG. 51 shows a nucleotide sequence (SEQ ID NO:51) of a native sequence PRO84746 cDNA, wherein SEQ ID NO:51 is a clone designated herein as “DNA329094”.

FIG. 52 shows the amino acid sequence (SEQ ID NO:52) derived from the coding sequence of SEQ ID NO:51 shown in FIG. 51.

FIG. 53 shows a nucleotide sequence (SEQ ID NO:53) of a native sequence PRO84223 cDNA, wherein SEQ ID NO:53 is a clone designated herein as “DNA328364”.

FIG. 54 shows the amino acid sequence (SEQ ID NO:54) derived from the coding sequence of SEQ ID NO:53 shown in FIG. 53.

FIG. 55 shows a nucleotide sequence (SEQ ID NO:55) of a native sequence PRO84670 cDNA, wherein SEQ ID NO:55 is a clone designated herein as “DNA328966”.

FIG. 56 shows the amino acid sequence (SEQ ID NO:56) derived from the coding sequence of SEQ ID NO:55 shown in FIG. 55.

FIG. 57 shows a nucleotide sequence (SEQ ID NO:57) of a native sequence PRO77352 cDNA, wherein SEQ ID NO:57 is a clone designated herein as “DNA329095”.

FIG. 58 shows the amino acid sequence (SEQ ID NO:58) derived from the coding sequence of SEQ ID NO:57 shown in FIG. 57.

FIG. 59 shows a nucleotide sequence (SEQ ID NO:59) of a native sequence PRO83815 cDNA, wherein SEQ ID NO:59 is a clone designated herein as “DNA327876”.

FIG. 60 shows the amino acid sequence (SEQ ID NO:60) derived from the coding sequence of SEQ ID NO:59 shown in FIG. 59.

FIG. 61 shows a nucleotide sequence (SEQ ID NO:61) of a native sequence PRO12926 cDNA, wherein SEQ ID NO:61 is a clone designated herein as “DNA153752”.

FIG. 62 shows the amino acid sequence (SEQ ID NO:62) derived from the coding sequence of SEQ ID NO:61 shown in FIG. 61.

FIG. 63 shows a nucleotide sequence (SEQ ID NO:63) of a native sequence PRO59084 cDNA, wherein SEQ ID NO:63 is a clone designated herein as “DNA270721”.

FIG. 64 shows the amino acid sequence (SEQ ID NO:64) derived from the coding sequence of SEQ ID NO:63 shown in FIG. 63.

FIG. 65 shows a nucleotide sequence (SEQ ID NO:65) of a native sequence PRO69520 cDNA, wherein SEQ ID NO:65 is a clone designated herein as “DNA287245”.

FIG. 66 shows the amino acid sequence (SEQ ID NO:66) derived from the coding sequence of SEQ ID NO:65 shown in FIG. 65.

FIG. 67 shows a nucleotide sequence (SEQ ID NO:67) of a native sequence PRO71134 cDNA, wherein SEQ ID NO:67 is a clone designated herein as “DNA327532”.

FIG. 68 shows the amino acid sequence (SEQ ID NO:68) derived from the coding sequence of SEQ ID NO:67 shown in FIG. 67.

FIG. 69 shows a nucleotide sequence (SEQ ID NO:69) of a native sequence PRO3632 cDNA, wherein SEQ ID NO:69 is a clone designated herein as “DNA97285”.

FIG. 70 shows the amino acid sequence (SEQ ID NO:70) derived from the coding sequence of SEQ ID NO:69 shown in FIG. 69.

FIG. 71 shows a nucleotide sequence (SEQ ID NO:71) of a native sequence PRO84747 cDNA, wherein SEQ ID NO:71 is a clone designated herein as “DNA329096”.

FIG. 72 shows the amino acid sequence (SEQ ID NO:72) derived from the coding sequence of SEQ ID NO:71 shown in FIG. 71.

FIG. 73 shows a nucleotide sequence (SEQ ID NO:73) of a native sequence PRO37518 cDNA, wherein SEQ ID NO:73 is a clone designated herein as “DNA227055”.

FIG. 74 shows the amino acid sequence (SEQ ID NO:74) derived from the coding sequence of SEQ ID NO:73 shown in FIG. 73.

FIG. 75 shows a nucleotide sequence (SEQ ID NO:75) of a native sequence PRO81277 cDNA, wherein SEQ ID NO:75 is a clone designated herein as “DNA324633”.

FIG. 76 shows the amino acid sequence (SEQ ID NO:76) derived from the coding sequence of SEQ ID NO:75 shown in FIG. 75.

FIG. 77 shows a nucleotide sequence (SEQ ID NO:77) of a native sequence PRO81277 cDNA, wherein SEQ ID NO:77 is a clone designated herein as “DNA324633”.

FIG. 78 shows the amino acid sequence (SEQ ID NO:78) derived from the coding sequence of SEQ ID NO:77 shown in FIG. 77.

FIG. 79 shows a nucleotide sequence (SEQ ID NO:79) of a native sequence PRO70258 cDNA, wherein SEQ ID NO:79 is a clone designated herein as “DNA324058”.

FIG. 80 shows the amino acid sequence (SEQ ID NO:80) derived from the coding sequence of SEQ ID NO:79 shown in FIG. 79.

FIG. 81 shows a nucleotide sequence (SEQ ID NO:81) of a native sequence PRO61271 cDNA, wherein SEQ ID NO:81 is a clone designated herein as “DNA327845”.

FIG. 82 shows the amino acid sequence (SEQ ID NO:82) derived from the coding sequence of SEQ ID NO:81 shown in FIG. 81.

FIG. 83 shows a nucleotide sequence (SEQ ID NO:83) of a native sequence PRO39268 cDNA, wherein SEQ ID NO:83 is a clone designated herein as “DNA287207”.

FIG. 84 shows the amino acid sequence (SEQ ID NO:84) derived from the coding sequence of SEQ ID NO:83 shown in FIG. 83.

FIG. 85 shows a nucleotide sequence (SEQ ID NO:85) of a native sequence PRO84748 cDNA, wherein SEQ ID NO:85 is a clone designated herein as “DNA329097”.

FIG. 86 shows the amino acid sequence (SEQ ID NO:86) derived from the coding sequence of SEQ ID NO:85 shown in FIG. 85.

FIG. 87 shows a nucleotide sequence (SEQ ID NO:87) of a native sequence PRO59895 cDNA, wherein SEQ ID NO:87 is a clone designated herein as “DNA271608”.

FIG. 88 shows the amino acid sequence (SEQ ID NO:88) derived from the coding sequence of SEQ ID NO:87 shown in FIG. 87.

FIG. 89 shows a nucleotide sequence (SEQ ID NO:89) of a native sequence PRO80773 cDNA, wherein SEQ ID NO:89 is a clone designated herein as “DNA324060”.

FIG. 90 shows the amino acid sequence (SEQ ID NO:90) derived from the coding sequence of SEQ ID NO:89 shown in FIG. 89.

FIG. 91 shows a nucleotide sequence (SEQ ID NO:91) of a native sequence PRO69492 cDNA, wherein SEQ ID NO:91 is a clone designated herein as “DNA287211”.

FIG. 92 shows the amino acid sequence (SEQ ID NO:92) derived from the coding sequence of SEQ ID NO:91 shown in FIG. 91.

FIG. 93 shows a nucleotide sequence (SEQ ID NO:93) of a native sequence PRO38258 cDNA, wherein SEQ ID NO:93 is a clone designated herein as “DNA227795”.

FIG. 94 shows the amino acid sequence (SEQ ID NO:94) derived from the coding sequence of SEQ ID NO:93 shown in FIG. 93.

FIG. 95 shows a nucleotide sequence (SEQ ID NO:95) of a native sequence PRO83005 cDNA, wherein SEQ ID NO:95 is a clone designated herein as “DNA326655”.

FIG. 96 shows the amino acid sequence (SEQ ID NO:96) derived from the coding sequence of SEQ ID NO:95 shown in FIG. 95.

FIG. 97 shows a nucleotide sequence (SEQ ID NO:97) of a native sequence PRO84749 cDNA, wherein SEQ ID NO:97 is a clone designated herein as “DNA329098”.

FIG. 98 shows the amino acid sequence (SEQ ID NO:98) derived from the coding sequence of SEQ ID NO:97 shown in FIG. 97.

FIG. 99 shows a nucleotide sequence (SEQ ID NO:99) of a native sequence PRO83581 cDNA, wherein SEQ ID NO:99 is a clone designated herein as “DNA327537”.

FIG. 100 shows the amino acid sequence (SEQ ID NO:100) derived from the coding sequence of SEQ ID NO:99 shown in FIG. 99.

FIG. 101 shows a nucleotide sequence (SEQ ID NO:101) of a native sequence PRO49642 cDNA, wherein SEQ ID NO:101 is a clone designated herein as “DNA254537”.

FIG. 102 shows the amino acid sequence (SEQ ID NO:102) derived from the coding sequence of SEQ ID NO:101 shown in FIG. 101.

FIG. 103 shows a nucleotide sequence (SEQ ID NO:103) of a native sequence PRO49675 cDNA, wherein SEQ ID NO:103 is a clone designated herein as “DNA254572”.

FIG. 104 shows the amino acid sequence (SEQ ID NO:104) derived from the coding sequence of SEQ ID NO:103 shown in FIG. 103.

FIG. 105 shows a nucleotide sequence (SEQ ID NO:105) of a native sequence PRO59358 cDNA, wherein SEQ ID NO:105 is a clone designated herein as “DNA271030”.

FIG. 106 shows the amino acid sequence (SEQ ID NO:106) derived from the coding sequence of SEQ ID NO:105 shown in FIG. 105.

FIG. 107 shows a nucleotide sequence (SEQ ID NO:107) of a native sequence PRO81477 cDNA, wherein SEQ ID NO:107 is a clone designated herein as “DNA324871”.

FIG. 108 shows the amino acid sequence (SEQ ID NO:108) derived from the coding sequence of SEQ ID NO:107 shown in FIG. 107.

FIG. 109 shows a nucleotide sequence (SEQ ID NO:109) of a native sequence PRO80814 cDNA, wherein SEQ ID NO:109 is a clone designated herein as “DNA324107”.

FIG. 110 shows the amino acid sequence (SEQ ID NO:110) derived from the coding sequence of SEQ ID NO:109 shown in FIG. 109.

FIG. 111 shows a nucleotide sequence (SEQ ID NO:111) of a native sequence PRO60127 cDNA, wherein SEQ ID NO:111 is a clone designated herein as “DNA329099”.

FIG. 112 shows the amino acid sequence (SEQ ID NO:112) derived from the coding sequence of SEQ ID NO:111 shown in FIG. 111.

FIG. 113 shows a nucleotide sequence (SEQ ID NO:113) of a native sequence PRO60127 cDNA, wherein SEQ ID NO:113 is a clone designated herein as “DNA271847”.

FIG. 114 shows the amino acid sequence (SEQ ID NO:114) derived from the coding sequence of SEQ ID NO:113 shown in FIG. 113.

FIG. 115 shows a nucleotide sequence (SEQ ID NO:115) of a native sequence PRO69473 cDNA, wherein SEQ ID NO:115 is a clone designated herein as “DNA287187”.

FIG. 116 shows the amino acid sequence (SEQ ID NO:116) derived from the coding sequence of SEQ ID NO:115 shown in FIG. 115.

FIG. 117 shows a nucleotide sequence (SEQ ID NO:117) of a native sequence PRO62041 cDNA, wherein SEQ ID NO:117 is a clone designated herein as “DNA274103”.

FIG. 118 shows the amino acid sequence (SEQ ID NO:118) derived from the coding sequence of SEQ ID NO:117 shown in FIG. 117.

FIG. 119 shows a nucleotide sequence (SEQ ID NO:119) of a native sequence cDNA, wherein SEQ ID NO:119 is a clone designated herein as “DNA328380”.

FIG. 120 shows a nucleotide sequence (SEQ ID NO:120) of a native sequence PRO61053 cDNA, wherein SEQ ID NO:120 is a clone designated herein as “DNA272974”.

FIG. 121 shows the amino acid sequence (SEQ ID NO:121) derived from the coding sequence of SEQ ID NO:120 shown in FIG. 120.

FIG. 122 shows a nucleotide sequence (SEQ ID NO:122) of a native sequence PRO57298 cDNA, wherein SEQ ID NO:122 is a clone designated herein as “DNA327255”.

FIG. 123 shows the amino acid sequence (SEQ ID NO:123) derived from the coding sequence of SEQ ID NO:122 shown in FIG. 122.

FIG. 124 shows a nucleotide sequence (SEQ ID NO:124) of a native sequence PRO38005 cDNA, wherein SEQ ID NO:124 is a clone designated herein as “DNA327540”.

FIG. 125 shows the amino acid sequence (SEQ ID NO:125) derived from the coding sequence of SEQ ID NO:124 shown in FIG. 124.

FIG. 126 shows a nucleotide sequence (SEQ ID NO:126) of a native sequence PRO36766 cDNA, wherein SEQ ID NO:126 is a clone designated herein as “DNA287217”.

FIG. 127 shows the amino acid sequence (SEQ ID NO:127) derived from the coding sequence of SEQ ID NO:126 shown in FIG. 126.

FIG. 128 shows a nucleotide sequence (SEQ ID NO:128) of a native sequence PRO84750 cDNA, wherein SEQ ID NO:128 is a clone designated herein as “DNA329100”.

FIG. 129 shows the amino acid sequence (SEQ ID NO:129) derived from the coding sequence of SEQ ID NO:128 shown in FIG. 128.

FIG. 130 shows a nucleotide sequence (SEQ ID NO:130) of a native sequence PRO82352 cDNA, wherein SEQ ID NO:130 is a clone designated herein as “DNA325896”.

FIG. 131 shows the amino acid sequence (SEQ ID NO:131) derived from the coding sequence of SEQ ID NO:130 shown in FIG. 130.

FIG. 132 shows a nucleotide sequence (SEQ ID NO:132) of a native sequence PRO71139 cDNA, wherein SEQ ID NO:132 is a clone designated herein as “DNA304713”.

FIG. 133 shows the amino acid sequence (SEQ ID NO:133) derived from the coding sequence of SEQ ID NO:132 shown in FIG. 132.

FIG. 134 shows a nucleotide sequence (SEQ ID NO:134) of a native sequence PRO2907 cDNA, wherein SEQ ID NO:134 is a clone designated herein as “DNA89242”.

FIG. 135 shows the amino acid sequence (SEQ ID NO:135) derived from the coding sequence of SEQ ID NO:134 shown in FIG. 134.

FIG. 136 shows a nucleotide sequence (SEQ ID NO:136) of a native sequence PRO84240 cDNA, wherein SEQ ID NO:136 is a clone designated herein as “DNA328388”.

FIG. 137 shows the amino acid sequence (SEQ ID NO:137) derived from the coding sequence of SEQ ID NO:136 shown in FIG. 136.

FIG. 138 shows a nucleotide sequence (SEQ ID NO:138) of a native sequence PRO11993 cDNA, wherein SEQ ID NO:138 is a clone designated herein as “DNA151697”.

FIG. 139 shows the amino acid sequence (SEQ ID NO:139) derived from the coding sequence of SEQ ID NO:138 shown in FIG. 138.

FIG. 140 shows a nucleotide sequence (SEQ ID NO:140) of a native sequence PRO84751 cDNA, wherein SEQ ID NO:140 is a clone designated herein as “DNA329101”.

FIG. 141 shows the amino acid sequence (SEQ ID NO:141) derived from the coding sequence of SEQ ID NO:140 shown in FIG. 140.

FIG. 142 shows a nucleotide sequence (SEQ ID NO:142) of a native sequence PRO69632 cDNA, wherein SEQ ID NO:142 is a clone designated herein as “DNA287372”.

FIG. 143 shows the amino acid sequence (SEQ ID NO:143) derived from the coding sequence of SEQ ID NO:142 shown in FIG. 142.

FIG. 144 shows a nucleotide sequence (SEQ ID NO:144) of a native sequence PRO81592 cDNA, wherein SEQ ID NO:144 is a clone designated herein as “DNA325001”.

FIG. 145 shows the amino acid sequence (SEQ ID NO:145) derived from the coding sequence of SEQ ID NO:144 shown in FIG. 144.

FIG. 146 shows a nucleotide sequence (SEQ ID NO:146) of a native sequence PRO83292 cDNA, wherein SEQ ID NO:146 is a clone designated herein as “DNA326984”.

FIG. 147 shows the amino acid sequence (SEQ ID NO:147) derived from the coding sequence of SEQ ID NO:146 shown in FIG. 146.

FIG. 148 shows a nucleotide sequence (SEQ ID NO:148) of a native sequence PRO615 cDNA, wherein SEQ ID NO:148 is a clone designated herein as “DNA329102”.

FIG. 149 shows the amino acid sequence (SEQ ID NO:149) derived from the coding sequence of SEQ ID NO:148 shown in FIG. 148.

FIG. 150 shows a nucleotide sequence (SEQ ID NO:150) of a native sequence PRO49824 cDNA, wherein SEQ ID NO:150 is a clone designated herein as “DNA254725”.

FIG. 151 shows the amino acid sequence (SEQ ID NO:151) derived from the coding sequence of SEQ ID NO:150 shown in FIG. 150.

FIG. 152 shows a nucleotide sequence (SEQ ID NO:152) of a native sequence PRO69484 cDNA, wherein SEQ ID NO:152 is a clone designated herein as “DNA287198”.

FIG. 153 shows the amino acid sequence (SEQ ID NO:153) derived from the coding sequence of SEQ ID NO:152 shown in FIG. 152.

FIG. 154 shows a nucleotide sequence (SEQ ID NO:154) of a native sequence PRO36173 cDNA, wherein SEQ ID NO:154 is a clone designated herein as “DNA225710”.

FIG. 155 shows the amino acid sequence (SEQ ID NO:155) derived from the coding sequence of SEQ ID NO:154 shown in FIG. 154.

FIG. 156 shows a nucleotide sequence (SEQ ID NO:156) of a native sequence PRO82678 cDNA, wherein SEQ ID NO:156 is a clone designated herein as “DNA326273”.

FIG. 157 shows the amino acid sequence (SEQ ID NO:157) derived from the coding sequence of SEQ ID NO:156 shown in FIG. 156.

FIG. 158 shows a nucleotide sequence (SEQ ID NO:158) of a native sequence PRO84752 cDNA, wherein SEQ ID NO:158 is a clone designated herein as “DNA329103”.

FIG. 159 shows the amino acid sequence (SEQ ID NO:159) derived from the coding sequence of SEQ ID NO:158 shown in FIG. 158.

FIG. 160 shows a nucleotide sequence (SEQ ID NO:160) of a native sequence PRO69550 cDNA, wherein SEQ ID NO:160 is a clone designated herein as “DNA329104”.

FIG. 161 shows the amino acid sequence (SEQ ID NO:161) derived from the coding sequence of SEQ ID NO:160 shown in FIG. 160.

FIG. 162 shows a nucleotide sequence (SEQ ID NO:162) of a native sequence PRO84753 cDNA, wherein SEQ ID NO:162 is a clone designated herein as “DNA329105”.

FIG. 163 shows the amino acid sequence (SEQ ID NO:163) derived from the coding sequence of SEQ ID NO:162 shown in FIG. 162.

FIG. 164 shows a nucleotide sequence (SEQ ID NO:164) of a native sequence PRO12890 cDNA, wherein SEQ ID NO:164 is a clone designated herein as “DNA151802”.

FIG. 165 shows the amino acid sequence (SEQ ID NO:165) derived from the coding sequence of SEQ ID NO:164 shown in FIG. 164.

FIG. 166 shows a nucleotide sequence (SEQ ID NO:166) of a native sequence PRO4780 cDNA, wherein SEQ ID NO:166 is a clone designated herein as “DNA103453”.

FIG. 167 shows the amino acid sequence (SEQ ID NO:167) derived from the coding sequence of SEQ ID NO:166 shown in FIG. 166.

FIG. 168 shows a nucleotide sequence (SEQ ID NO:168) of a native sequence PRO60513 cDNA, wherein SEQ ID NO:168 is a clone designated herein as “DNA272251”.

FIG. 169 shows the amino acid sequence (SEQ ID NO:169) derived from the coding sequence of SEQ ID NO:168 shown in FIG. 168.

FIG. 170 shows a nucleotide sequence (SEQ ID NO:170) of a native sequence PRO61616 cDNA, wherein SEQ ID NO:170 is a clone designated herein as “DNA273645”.

FIG. 171 shows the amino acid sequence (SEQ ID NO:171) derived from the coding sequence of SEQ ID NO:170 shown in FIG. 170.

FIG. 172 shows a nucleotide sequence (SEQ ID NO:172) of a native sequence PRO69463 cDNA, wherein SEQ ID NO:172 is a clone designated herein as “DNA287173”.

FIG. 173 shows the amino acid sequence (SEQ ID NO:173) derived from the coding sequence of SEQ ID NO:172 shown in FIG. 172.

FIG. 174 shows a nucleotide sequence (SEQ ID NO:174) of a native sequence PRO69595 cDNA, wherein SEQ ID NO:174 is a clone designated herein as “DNA287331”.

FIG. 175 shows the amino acid sequence (SEQ ID NO:175) derived from the coding sequence of SEQ ID NO:174 shown in FIG. 174.

FIG. 176 shows a nucleotide sequence (SEQ ID NO:176) of a native sequence PRO62075 cDNA, wherein SEQ ID NO:176 is a clone designated herein as “DNA274139”.

FIG. 177 shows the amino acid sequence (SEQ ID NO:177) derived from the coding sequence of SEQ ID NO:176 shown in FIG. 176.

FIG. 178 shows a nucleotide sequence (SEQ ID NO:178) of a native sequence PRO59281 cDNA, wherein SEQ ID NO:178 is a clone designated herein as “DNA270950”.

FIG. 179 shows the amino acid sequence (SEQ ID NO:179) derived from the coding sequence of SEQ ID NO:178 shown in FIG. 178.

FIG. 180 shows a nucleotide sequence (SEQ ID NO:180) of a native sequence PRO81979 cDNA, wherein SEQ ID NO:180 is a clone designated herein as “DNA329004”.

FIG. 181 shows the amino acid sequence (SEQ ID NO:181) derived from the coding sequence of SEQ ID NO:180 shown in FIG. 180.

FIG. 182 shows a nucleotide sequence (SEQ ID NO:182) of a native sequence PRO84252 cDNA, wherein SEQ ID NO:182 is a clone designated herein as “DNA328405”.

FIG. 183 shows the amino acid sequence (SEQ ID NO:183) derived from the coding sequence of SEQ ID NO:182 shown in FIG. 182.

FIG. 184 shows a nucleotide sequence (SEQ ID NO:184) of a native sequence PRO83360 cDNA, wherein SEQ ID NO:184 is a clone designated herein as “DNA329106”.

FIG. 185 shows the amino acid sequence (SEQ ID NO:185) derived from the coding sequence of SEQ ID NO:184 shown in FIG. 184.

FIG. 186 shows a nucleotide sequence (SEQ ID NO:186) of a native sequence PRO71133 cDNA, wherein SEQ ID NO:186 is a clone designated herein as “DNA304707”.

FIG. 187 shows the amino acid sequence (SEQ ID NO:187) derived from the coding sequence of SEQ ID NO:186 shown in FIG. 186.

FIG. 188 shows a nucleotide sequence (SEQ ID NO:188) of a native sequence PRO62518 cDNA, wherein SEQ ID NO188: is a clone designated herein as “DNA274745”.

FIG. 189 shows the amino acid sequence (SEQ ID NO:189) derived from the coding sequence of SEQ ID NO:188 shown in FIG. 188.

FIG. 190 shows a nucleotide sequence (SEQ ID NO:190) of a native sequence PRO4912 cDNA, wherein SEQ ID NO:190 is a clone designated herein as “DNA329002”.

FIG. 191 shows the amino acid sequence (SEQ ID NO:191) derived from the coding sequence of SEQ ID NO:190 shown in FIG. 190.

FIG. 192 shows a nucleotide sequence (SEQ ID NO:192) of a native sequence PRO4912 cDNA, wherein SEQ ID NO:192 is a clone designated herein as “DNA329002”.

FIG. 193 shows the amino acid sequence (SEQ ID NO:193) derived from the coding sequence of SEQ ID NO:192 shown in FIG. 192.

FIG. 194 shows a nucleotide sequence (SEQ ID NO:194) of a native sequence PRO59943 cDNA, wherein SEQ ID NO:194 is a clone designated herein as “DNA271656”.

FIG. 195 shows the amino acid sequence (SEQ ID NO:195) derived from the coding sequence of SEQ ID NO:194 shown in FIG. 194.

FIG. 196 shows a nucleotide sequence (SEQ ID NO:196) of a native sequence PRO84754 cDNA, wherein SEQ ID NO:196 is a clone designated herein as “DNA329107”.

FIG. 197 shows the amino acid sequence (SEQ ID NO:197) derived from the coding sequence of SEQ ID NO:196 shown in FIG. 196.

FIG. 198 shows a nucleotide sequence (SEQ ID NO:198) of a native sequence PRO84755 cDNA, wherein SEQ ID NO:198 is a clone designated herein as “DNA329108”.

FIG. 199 shows the amino acid sequence (SEQ ID NO:199) derived from the coding sequence of SEQ ID NO:198 shown in FIG. 198.

FIG. 200 shows a nucleotide sequence (SEQ ID NO:200) of a native sequence PRO81854 cDNA, wherein SEQ ID NO:200 is a clone designated herein as “DNA329109”.

FIG. 201 shows the amino acid sequence (SEQ ID NO:201) derived from the coding sequence of SEQ ID NO:200 shown in FIG. 200.

FIG. 202 shows a nucleotide sequence (SEQ ID NO:202) of a native sequence PRO11586 cDNA, wherein SEQ ID NO:202 is a clone designated herein as “DNA3291 10”.

FIG. 203 shows the amino acid sequence (SEQ ID NO:203) derived from the coding sequence of SEQ ID NO:202 shown in FIG. 202.

FIG. 204 shows a nucleotide sequence (SEQ ID NO:204) of a native sequence PRO cDNA, wherein SEQ ID NO:204 is a clone designated herein as “DNA”.

FIG. 205 shows the amino acid sequence (SEQ ID NO:205) derived from the coding sequence of SEQ ID NO:204 shown in FIG. 204.

FIG. 206 shows a nucleotide sequence (SEQ ID NO:206) of a native sequence PRO59309 cDNA, wherein SEQ ID NO:206 is a clone designated herein as “DNA270979”.

FIG. 207 shows the amino acid sequence (SEQ ID NO:207) derived from the coding sequence of SEQ ID NO:206 shown in FIG. 206.

FIG. 208 shows a nucleotide sequence (SEQ ID NO:208) of a native sequence PRO2338 cDNA, wherein SEQ ID NO:208 is a clone designated herein as “DNA88418”.

FIG. 209 shows the amino acid sequence (SEQ ID NO:209) derived from the coding sequence of SEQ ID NO:208 shown in FIG. 208.

FIG. 210 shows a nucleotide sequence (SEQ ID NO:210) of a native sequence PRO37063 cDNA, wherein SEQ ID NO:210 is a clone designated herein as “DNA226600”.

FIG. 211 shows the amino acid sequence (SEQ ID NO:211) derived from the coding sequence of SEQ ID NO:210 shown in FIG. 210.

FIG. 212 shows a nucleotide sequence (SEQ ID NO:212) of a native sequence PRO84757 cDNA, wherein SEQ ID NO:212 is a clone designated herein as “DNA329112”.

FIG. 213 shows the amino acid sequence (SEQ ID NO:213) derived from the coding sequence of SEQ ID NO:212 shown in FIG. 212.

FIG. 214 shows a nucleotide sequence (SEQ ID NO:214) of a native sequence PRO83076 cDNA, wherein SEQ ID NO:214 is a clone designated herein as “DNA326736”.

FIG. 215 shows the amino acid sequence (SEQ ID NO:215) derived from the coding sequence of SEQ ID NO:214 shown in FIG. 214.

FIG. 216 shows a nucleotide sequence (SEQ ID NO:216) of a native sequence PRO49881 cDNA, wherein SEQ ID NO:216 is a clone designated herein as “DNA254783”.

FIG. 217 shows the amino acid sequence (SEQ ID NO:217) derived from the coding sequence of SEQ ID NO:216 shown in FIG. 216.

FIG. 218 shows a nucleotide sequence (SEQ ID NO:218) of a native sequence PRO37073 cDNA, wherein SEQ ID NO:218 is a clone designated herein as “DNA304459”.

FIG. 219 shows the amino acid sequence (SEQ ID NO:219) derived from the coding sequence of SEQ ID NO:218 shown in FIG. 218.

FIG. 220 shows a nucleotide sequence (SEQ ID NO:220) of a native sequence PRO37073 cDNA, wherein SEQ ID NO:220 is a clone designated herein as “DNA304459”.

FIG. 221 shows the amino acid sequence (SEQ ID NO:221) derived from the coding sequence of SEQ ID NO:220 shown in FIG. 220.

FIG. 222 shows a nucleotide sequence (SEQ ID NO:222) of a native sequence PRO49210 cDNA, wherein SEQ ID NO:222 is a clone designated herein as “DNA253807”.

FIG. 223 shows the amino acid sequence (SEQ ID NO:223) derived from the coding sequence of SEQ ID NO:222 shown in FIG. 222.

FIG. 224 shows a nucleotide sequence (SEQ ID NO:224) of a native sequence PRO80498 cDNA, wherein SEQ ID NO:224 is a clone designated herein as “DNA323741”.

FIG. 225 shows the amino acid sequence (SEQ ID NO:225) derived from the coding sequence of SEQ ID NO:224 shown in FIG. 224.

FIG. 226 shows a nucleotide sequence (SEQ ID NO:226) of a native sequence PRO83586 cDNA, wherein SEQ ID NO:226 is a clone designated herein as “DNA327555”.

FIG. 227 shows the amino acid sequence (SEQ ID NO:227) derived from the coding sequence of SEQ ID NO:226 shown in FIG. 226.

FIG. 228 shows a nucleotide sequence (SEQ ID NO:228) of a native sequence PRO3647 cDNA, wherein SEQ ID NO:228 is a clone designated herein as “DNA97300”.

FIG. 229 shows the amino acid sequence (SEQ ID NO:229) derived from the coding sequence of SEQ ID NO:228 shown in FIG. 228.

FIG. 230 shows a nucleotide sequence (SEQ ID NO:231) of a native sequence PRO84262 cDNA, wherein SEQ ID NO:230 is a clone designated herein as “DNA328419”.

FIG. 231 shows the amino acid sequence (SEQ ID NO:231) derived from the coding sequence of SEQ ID NO:230 shown in FIG. 230.

FIG. 232 shows a nucleotide sequence (SEQ ID NO:232) of a native sequence PRO37941 cDNA, wherein SEQ ID NO:232 is a clone designated herein as “DNA227478”.

FIG. 233 shows the amino acid sequence (SEQ ID NO:233) derived from the coding sequence of SEQ ID NO:232 shown in FIG. 232.

FIG. 234 shows a nucleotide sequence (SEQ ID NO:234) of a native sequence PRO59324 cDNA, wherein SEQ ID NO:234 is a clone designated herein as “DNA270995”.

FIG. 235 shows the amino acid sequence (SEQ ID NO:235) derived from the coding sequence of SEQ ID NO:234 shown in FIG. 234.

FIG. 236 shows a nucleotide sequence (SEQ ID NO:236) of a native sequence PRO37534 cDNA, wherein SEQ ID NO:236 is a clone designated herein as “DNA227071”.

FIG. 237 shows the amino acid sequence (SEQ ID NO:237) derived from the coding sequence of SEQ ID NO:236 shown in FIG. 236.

FIG. 238 shows a nucleotide sequence (SEQ ID NO:238) of a native sequence PRO84758 cDNA, wherein SEQ ID NO:238 is a clone designated herein as “DNA329113”.

FIG. 239 shows the amino acid sequence (SEQ ID NO:239) derived from the coding sequence of SEQ ID NO:238 shown in FIG. 238.

FIG. 240 shows a nucleotide sequence (SEQ ID NO:240) of a native sequence PRO70425 cDNA, wherein SEQ ID NO:240 is a clone designated herein as “DNA290280”.

FIG. 241 shows the amino acid sequence (SEQ ID NO:241) derived from the coding sequence of SEQ ID NO:240 shown in FIG. 240.

FIG. 242 shows a nucleotide sequence (SEQ ID NO:242) of a native sequence PRO60991 cDNA, wherein SEQ ID NO:242 is a clone designated herein as “DNA272904”.

FIG. 243 shows the amino acid sequence (SEQ ID NO:243) derived from the coding sequence of SEQ ID NO:242 shown in FIG. 242.

FIG. 244 shows a nucleotide sequence (SEQ ID NO:244) of a native sequence PRO84759 cDNA, wherein SEQ ID NO:244 is a clone designated herein as “DNA329114”.

FIG. 245 shows the amino acid sequence (SEQ ID NO:245) derived from the coding sequence of SEQ ID NO:244 shown in FIG. 244.

FIG. 246 shows a nucleotide sequence (SEQ ID NO:246) of a native sequence PRO84760 cDNA, wherein SEQ ID NO:246 is a clone designated herein as “DNA329115”.

FIG. 247 shows the amino acid sequence (SEQ ID NO:247) derived from the coding sequence of SEQ ID NO:246 shown in FIG. 246.

FIG. 248 shows a nucleotide sequence (SEQ ID NO:248) of a native sequence PRO34726 cDNA, wherein SEQ ID NO:248 is a clone designated herein as “DNA220748”.

FIG. 249 shows the amino acid sequence (SEQ ID NO:249) derived from the coding sequence of SEQ ID NO:248 shown in FIG. 248.

FIG. 250 shows a nucleotide sequence (SEQ ID NO:250) of a native sequence PRO84761 cDNA, wherein SEQ ID NO:250 is a clone designated herein as “DNA329116”.

FIG. 251 shows the amino acid sequence (SEQ ID NO:251) derived from the coding sequence of SEQ ID NO:250 shown in FIG. 250.

FIG. 252 shows a nucleotide sequence (SEQ ID NO:252) of a native sequence cDNA, wherein SEQ ID NO:252 is a clone designated herein as “DNA329117”.

FIG. 253 shows a nucleotide sequence (SEQ ID NO:253) of a native sequence PRO37335 cDNA, wherein SEQ ID NO:253 is a clone designated herein as “DNA226872”.

FIG. 254 shows the amino acid sequence (SEQ ID NO:254) derived from the coding sequence of SEQ ID NO:253 shown in FIG. 253.

FIG. 255 shows a nucleotide sequence (SEQ ID NO:255) of a native sequence PRO37924 cDNA, wherein SEQ ID NO:255 is a clone designated herein as “DNA227461”.

FIG. 256 shows the amino acid sequence (SEQ ID NO:256) derived from the coding sequence of SEQ ID NO:255 shown in FIG. 255.

FIG. 257 shows a nucleotide sequence (SEQ ID NO:257) of a native sequence PRO38039 cDNA, wherein SEQ ID NO:257 is a clone designated herein as “DNA227576”.

FIG. 258 shows the amino acid sequence (SEQ ID NO:258) derived from the coding sequence of SEQ ID NO:257 shown in FIG. 257.

FIG. 259 shows a nucleotide sequence (SEQ ID NO:259) of a native sequence PRO82769 cDNA, wherein SEQ ID NO:259 is a clone designated herein as “DNA326373”.

FIG. 260 shows the amino acid sequence (SEQ ID NO:260) derived from the coding sequence of SEQ ID NO:259 shown in FIG. 259.

FIG. 261 shows a nucleotide sequence (SEQ ID NO:261) of a native sequence PRO83589 cDNA, wherein SEQ ID NO:261 is a clone designated herein as “DNA327559”.

FIG. 262 shows the amino acid sequence (SEQ ID NO:262) derived from the coding sequence of SEQ ID NO:261 shown in FIG. 261.

FIG. 263 shows a nucleotide sequence (SEQ ID NO:263) of a native sequence PRO59136 cDNA, wherein SEQ ID NO:263 is a clone designated herein as “DNA287167”.

FIG. 264 shows the amino acid sequence (SEQ ID NO:264) derived from the coding sequence of SEQ ID NO:263 shown in FIG. 263.

FIG. 265 shows a nucleotide sequence (SEQ ID NO:265) of a native sequence PRO69491 cDNA, wherein SEQ ID NO:265 is a clone designated herein as “DNA287625”.

FIG. 266 shows the amino acid sequence (SEQ ID NO:266) derived from the coding sequence of SEQ ID NO:265 shown in FIG. 265.

FIG. 267 shows a nucleotide sequence (SEQ ID NO:267) of a native sequence PRO80735 cDNA, wherein SEQ ID NO:267 is a clone designated herein as “DNA324015”.

FIG. 268 shows the amino acid sequence (SEQ ID NO:268) derived from the coding sequence of SEQ ID NO:267 shown in FIG. 267.

FIG. 269 shows a nucleotide sequence (SEQ ID NO:269) of a native sequence PRO83123 cDNA, wherein SEQ ID NO:269 is a clone designated herein as “DNA329118”.

FIG. 270 shows the amino acid sequence (SEQ ID NO:270) derived from the coding sequence of SEQ ID NO:269 shown in FIG. 269.

FIG. 271 shows a nucleotide sequence (SEQ ID NO:271) of a native sequence PRO2842 cDNA, wherein SEQ ID NO:271 is a clone designated herein as “DNA88562”.

FIG. 272 shows the amino acid sequence (SEQ ID NO:272) derived from the coding sequence of SEQ ID NO:271 shown in FIG. 271.

FIG. 273 shows a nucleotide sequence (SEQ ID NO:273) of a native sequence PRO60337 cDNA, wherein SEQ ID NO:273 is a clone designated herein as “DNA272066”.

FIG. 274 shows the amino acid sequence (SEQ ID NO:274) derived from the coding sequence of SEQ ID NO:273 shown in FIG. 273.

FIG. 275 shows a nucleotide sequence (SEQ ID NO:275) of a native sequence PRO11583 cDNA, wherein SEQ ID NO:275 is a clone designated herein as “DNA150805”.

FIG. 276 shows the amino acid sequence (SEQ ID NO:276) derived from the coding sequence of SEQ ID NO:275 shown in FIG. 275.

FIG. 277 shows a nucleotide sequence (SEQ ID NO:277) of a native sequence PRO80988 cDNA, wherein SEQ ID NO:277 is a clone designated herein as “DNA324310”.

FIG. 278 shows the amino acid sequence (SEQ ID NO:278) derived from the coding sequence of SEQ ID NO:277 shown in FIG. 277.

FIG. 279 shows a nucleotide sequence (SEQ ID NO:279) of a native sequence PRO63048 cDNA, wherein SEQ ID NO:279 is a clone designated herein as “DNA275385”.

FIG. 280 shows the amino acid sequence (SEQ ID NO:280) derived from the coding sequence of SEQ ID NO:279 shown in FIG. 279.

FIG. 281 shows a nucleotide sequence (SEQ ID NO:281) of a native sequence PRO37575 cDNA, wherein SEQ ID NO:281 is a clone designated herein as “DNA2271 12”.

FIG. 282 shows the amino acid sequence (SEQ ID NO:282) derived from the coding sequence of SEQ ID NO:281 shown in FIG. 281.

FIG. 283 shows a nucleotide sequence (SEQ ID NO:283) of a native sequence PRO62927 cDNA, wherein SEQ ID NO:283 is a clone designated herein as “DNA275240”.

FIG. 284 shows the amino acid sequence (SEQ ID NO:284) derived from the coding sequence of SEQ ID NO:283 shown in FIG. 283.

FIG. 285 shows a nucleotide sequence (SEQ ID NO:285) of a native sequence PRO4554 cDNA, wherein SEQ ID NO:285 is a clone designated herein as “DNA329119”.

FIG. 286 shows the amino acid sequence (SEQ ID NO:286) derived from the coding sequence of SEQ ID NO:285 shown in FIG. 285.

FIG. 287 shows a nucleotide sequence (SEQ ID NO:287) of a native sequence PRO2752 cDNA, wherein SEQ ID NO:287 is a clone designated herein as “DNA329120”.

FIG. 288 shows the amino acid sequence (SEQ ID NO:288) derived from the coding sequence of SEQ ID NO:287 shown in FIG. 287.

FIG. 289 shows a nucleotide sequence (SEQ ID NO:289) of a native sequence PRO62097 cDNA, wherein SEQ ID NO:289 is a clone designated herein as “DNA274167”.

FIG. 290 shows the amino acid sequence (SEQ ID NO:290) derived from the coding sequence of SEQ ID NO:289 shown in FIG. 289.

FIG. 291 shows a nucleotide sequence (SEQ ID NO:291) of a native sequence PRO62908 cDNA, wherein SEQ ID NO:291 is a clone designated herein as “DNA275214”.

FIG. 292 shows the amino acid sequence (SEQ ID NO:292) derived from the coding sequence of SEQ ID NO:291 shown in FIG. 291.

FIG. 293 shows a nucleotide sequence (SEQ ID NO:293) of a native sequence PRO8359.6 cDNA, wherein SEQ ID NO:293 is a clone designated herein as “DNA327567”.

FIG. 294 shows the amino acid sequence (SEQ ID NO:294) derived from the coding sequence of SEQ ID NO:293 shown in FIG. 293.

FIG. 295 shows a nucleotide sequence (SEQ ID NO:295) of a native sequence PRO36579 cDNA, wherein SEQ ID NO:295 is a clone designated herein as “DNA226116”.

FIG. 296 shows the amino acid sequence (SEQ ID NO:296) derived from the coding sequence of SEQ ID NO:295 shown in FIG. 295.

FIG. 297 shows a nucleotide sequence (SEQ ID NO:297) of a native sequence PRO60487 cDNA, wherein SEQ ID NO:297 is a clone designated herein as “DNA272225”.

FIG. 298 shows the amino acid sequence (SEQ ID NO:298) derived from the coding sequence of SEQ ID NO:297 shown in FIG. 297.

FIG. 299 shows a nucleotide sequence (SEQ ID NO:299) of a native sequence PRO84274 cDNA, wherein SEQ ID NO:299 is a clone designated herein as “DNA328440”.

FIG. 300 shows the amino acid sequence (SEQ ID NO:300) derived from the coding sequence of SEQ ID NO:299 shown in FIG. 299.

FIG. 301 shows a nucleotide sequence (SEQ ID NO:301) of a native sequence PRO84695 cDNA, wherein SEQ ID NO:301 is a clone designated herein as “DNA329020”.

FIG. 302 shows the amino acid sequence (SEQ ID NO:302) derived from the coding sequence of SEQ ID NO:301 shown in FIG. 301.

FIG. 303 shows a nucleotide sequence (SEQ ID NO:303) of a native sequence PRO84275 cDNA, wherein SEQ ID NO:303 is a clone designated herein as “DNA328442”.

FIG. 304 shows the amino acid sequence (SEQ ID NO:304) derived from the coding sequence of SEQ ID NO:303 shown in FIG. 303.

FIG. 305 shows a nucleotide sequence (SEQ ID NO:305) of a native sequence PRO49673 cDNA, wherein SEQ ID NO:305 is a clone designated herein as “DNA254570”.

FIG. 306 shows the amino acid sequence (SEQ ID NO:306) derived from the coding sequence of SEQ ID NO:305 shown in FIG. 305.

FIG. 307 shows a nucleotide sequence (SEQ ID NO:307) of a native sequence PRO84763 cDNA, wherein SEQ ID NO:307 is a clone designated herein as “DNA329121”.

FIG. 308 shows the amino acid sequence (SEQ ID NO:308) derived from the coding sequence of SEQ ID NO:307 shown in FIG. 307.

FIG. 309 shows a nucleotide sequence (SEQ ID NO:309) of a native sequence PRO84277 cDNA, wherein SEQ ID NO:309 is a clone designated herein as “DNA328444”.

FIG. 310 shows the amino acid sequence (SEQ ID NO:310) derived from the coding sequence of SEQ ID NO:309 shown in FIG. 309.

FIG. 311 shows a nucleotide sequence (SEQ ID NO:311) of a native sequence PRO62362 cDNA, wherein SEQ ID NO:311 is a clone designated herein as “DNA328448”.

FIG. 312 shows the amino acid sequence (SEQ ID NO:312) derived from the coding sequence of SEQ ID NO:311 shown in FIG. 311.

FIG. 313 shows a nucleotide sequence (SEQ ID NO:313) of a native sequence PRO81689 cDNA, wherein SEQ ID NO:313 is a clone designated herein as “DNA325115”.

FIG. 314 shows the amino acid sequence (SEQ ID NO:314) derived from the coding sequence of SEQ ID NO:313 shown in FIG. 313.

FIG. 315 shows a nucleotide sequence (SEQ ID NO:315) of a native sequence PRO58880 cDNA, wherein SEQ ID NO:315 is a clone designated herein as “DNA270502”.

FIG. 316 shows the amino acid sequence (SEQ ID NO:316) derived from the coding sequence of SEQ ID NO:315 shown in FIG. 315.

FIG. 317 shows a nucleotide sequence (SEQ ID NO:317) of a native sequence PRO1213 cDNA, wherein SEQ ID NO:317 is a clone designated herein as “DNA66487”.

FIG. 318 shows the amino acid sequence (SEQ ID NO:318) derived from the coding sequence of SEQ ID NO:317 shown in FIG. 317.

FIG. 319 shows a nucleotide sequence (SEQ ID NO:319) of a native sequence PRO83600 cDNA, wherein SEQ ID NO:319 is a clone designated herein as “DNA327576”.

FIG. 320 shows the amino acid sequence (SEQ ID NO:320) derived from the coding sequence of SEQ ID NO:319 shown in FIG. 319.

FIG. 321 shows a nucleotide sequence (SEQ ID NO:321) of a native sequence PRO21744 cDNA, wherein SEQ ID NO:321 is a clone designated herein as “DNA188225”.

FIG. 322 shows the amino acid sequence (SEQ ID NO:322) derived from the coding sequence of SEQ ID NO:321 shown in FIG. 321.

FIG. 323 shows a nucleotide sequence (SEQ ID NO:323) of a native sequence PRO84764 cDNA, wherein SEQ ID NO:323 is a clone designated herein as “DNA329122”.

FIG. 324 shows the amino acid sequence (SEQ ID NO:324) derived from the coding sequence of SEQ ID NO:323 shown in FIG. 323.

FIG. 325 shows a nucleotide sequence (SEQ ID NO:325) of a native sequence PRO84765 cDNA, wherein SEQ ID NO:325 is a clone designated herein as “DNA329123”.

FIG. 326 shows the amino acid sequence (SEQ ID NO:326) derived from the coding sequence of SEQ ID NO:325 shown in FIG. 325.

FIG. 327 shows a nucleotide sequence (SEQ ID NO:327) of a native sequence PRO84766 cDNA, wherein SEQ ID NO:327 is a clone designated herein as “DNA329124”.

FIG. 328 shows the amino acid sequence (SEQ ID NO:328) derived from the coding sequence of SEQ ID NO:327 shown in FIG. 327.

FIG. 329 shows a nucleotide sequence (SEQ ID NO:329) of a native sequence PRO64556 cDNA, wherein SEQ ID NO:329 is a clone designated herein as “DNA277809”.

FIG. 330 shows the amino acid sequence (SEQ ID NO:330) derived from the coding sequence of SEQ ID NO:329 shown in FIG. 329.

FIG. 331 shows a nucleotide sequence (SEQ ID NO:331) of a native sequence PRO83257 cDNA, wherein SEQ ID NO:331 is a clone designated herein as “DNA326939”.

FIG. 332 shows the amino acid sequence (SEQ ID NO:332) derived from the coding sequence of SEQ ID NO:331 shown in FIG. 331.

FIG. 333 shows a nucleotide sequence (SEQ ID NO:333) of a native sequence PRO71111 cDNA, wherein SEQ ID NO:333 is a clone designated herein as “DNA304685”.

FIG. 334 shows the amino acid sequence (SEQ ID NO:334) derived from the coding sequence of SEQ ID NO:333 shown in FIG. 333.

FIG. 335 shows a nucleotide sequence (SEQ ID NO:335) of a native sequence PRO84767 cDNA, wherein SEQ ID NO:335 is a clone designated herein as “DNA329125”.

FIG. 336 shows the amino acid sequence (SEQ ID NO:336) derived from the coding sequence of SEQ ID NO: shown in Figure.

FIG. 337 shows a nucleotide sequence (SEQ ID NO:337) of a native sequence PRO62626 cDNA, wherein SEQ ID NO:337 is a clone designated herein as “DNA274881”.

FIG. 338 shows the amino acid sequence (SEQ ID NO:338) derived from the coding sequence of SEQ ID NO:337 shown in FIG. 337.

FIG. 339 shows a nucleotide sequence (SEQ ID NO:339) of a native sequence PRO84768 cDNA, wherein SEQ ID NO:339 is a clone designated herein as “DNA329126”.

FIG. 340 shows the amino acid sequence (SEQ ID NO:340) derived from the coding sequence of SEQ ID NO:339 shown in FIG. 339.

FIG. 341 shows a nucleotide sequence (SEQ ID NO:341) of a native sequence PRO49244 cDNA, wherein SEQ ID NO:341 is a clone designated herein as “DNA254129”.

FIG. 342 shows the amino acid sequence (SEQ ID NO:342) derived from the coding sequence of SEQ ID NO:341 shown in FIG. 341.

FIG. 343 shows a nucleotide sequence (SEQ ID NO:343) of a native sequence PRO60906 cDNA, wherein SEQ ID NO:343 is a clone designated herein as “DNA272801”.

FIG. 344 shows the amino acid sequence (SEQ ID NO:344) derived from the coding sequence of SEQ ID NO:343 shown in FIG. 343.

FIG. 345 shows a nucleotide sequence (SEQ ID NO:345) of a native sequence PRO62479 cDNA, wherein SEQ ID NO:345 is a clone designated herein as “DNA274690”.

FIG. 346 shows the amino acid sequence (SEQ ID NO:346) derived from the coding sequence of SEQ ID NO:345 shown in FIG. 345.

FIG. 347 shows a nucleotide sequence (SEQ ID NO:347) of a native sequence PRO81599 cDNA, wherein SEQ ID NO:347 is a clone designated herein as “DNA329127”.

FIG. 348 shows the amino acid sequence (SEQ ID NO:348) derived from the coding sequence of SEQ ID NO:347 shown in FIG. 347.

FIG. 349 shows a nucleotide sequence (SEQ ID NO:349) of a native sequence PRO84769 cDNA, wherein SEQ ID NO:349 is a clone designated herein as “DNA329128”.

FIG. 350 shows the amino acid sequence (SEQ ID NO:350) derived from the coding sequence of SEQ ID NO:349 shown in FIG. 349.

FIG. 351 shows a nucleotide sequence (SEQ ID NO:351) of a native sequence PRO60248 cDNA, wherein SEQ ID NO:351 is a clone designated herein as “DNA271973”.

FIG. 352 shows the amino acid sequence (SEQ ID NO:352) derived from the coding sequence of SEQ ID NO:351 shown in FIG. 351.

FIG. 353 shows a nucleotide sequence (SEQ ID NO:353) of a native sequence PRO84288 cDNA, wherein SEQ ID NO:353 is a clone designated herein as “DNA329129”.

FIG. 354 shows the amino acid sequence (SEQ ID NO:354) derived from the coding sequence of SEQ ID NO:353 shown in FIG. 353.

FIG. 355 shows a nucleotide sequence (SEQ ID NO:355) of a native sequence PRO61349 cDNA, wherein SEQ ID NO:355 is a clone designated herein as “DNA273346”.

FIG. 356 shows the amino acid sequence (SEQ ID NO:356) derived from the coding sequence of SEQ ID NO:355 shown in FIG. 355.

FIG. 357 shows a nucleotide sequence (SEQ ID NO:357) of a native sequence PRO12742 cDNA, wherein SEQ ID NO:357 is a clone designated herein as “DNA151878”.

FIG. 358 shows the amino acid sequence (SEQ ID NO:358) derived from the coding sequence of SEQ ID NO:357 shown in FIG. 357.

FIG. 359 shows a nucleotide sequence (SEQ ID NO:359) of a native sequence PRO60936 cDNA, wherein SEQ ID NO:359 is a clone designated herein as “DNA272840”.

FIG. 360 shows the amino acid sequence (SEQ ID NO:360) derived from the coding sequence of SEQ ID NO:359 shown in FIG. 359.

FIG. 361 shows a nucleotide sequence (SEQ ID NO:361) of a native sequence PRO34252 cDNA, wherein SEQ ID NO:361 is a clone designated herein as “DNA216500”.

FIG. 362 shows the amino acid sequence (SEQ ID NO:362) derived from the coding sequence of SEQ ID NO:361 shown in FIG. 361.

FIG. 363 shows a nucleotide sequence (SEQ ID NO:363) of a native sequence PRO20124 cDNA, wherein SEQ ID NO:363 is a clone designated herein as “DNA329130”.

FIG. 364 shows the amino acid sequence (SEQ ID NO:364) derived from the coding sequence of SEQ ID NO:363 shown in FIG. 363.

FIG. 365 shows a nucleotide sequence (SEQ ID NO:365) of a native sequence PRO84770 cDNA, wherein SEQ ID NO:365 is a clone designated herein as “DNA329131”.

FIG. 366 shows the amino acid sequence (SEQ ID NO:366) derived from the coding sequence of SEQ ID NO:365 shown in FIG. 365.

FIG. 367 shows a nucleotide sequence (SEQ ID NO:367) of a native sequence PRO81877 cDNA, wherein SEQ ID NO:367 is a clone designated herein as “DNA325334”.

FIG. 368 shows the amino acid sequence (SEQ ID NO:368) derived from the coding sequence of SEQ ID NO:367 shown in FIG. 367.

FIG. 369 shows a nucleotide sequence (SEQ ID NO:369) of a native sequence PRO60742 cDNA, wherein SEQ ID NO:369 is a clone designated herein as “DNA272608”.

FIG. 370 shows the amino acid sequence (SEQ ID NO:370) derived from the coding sequence of SEQ ID NO:369 shown in FIG. 369.

FIG. 371 shows a nucleotide sequence (SEQ ID NO:371) of a native sequence PRO-80649 cDNA, wherein SEQ ID NO:371 is a clone designated herein as “DNA327584”.

FIG. 372 shows the amino acid sequence (SEQ ID NO:372) derived from the coding sequence of SEQ ID NO:371 shown in FIG. 371.

FIG. 373 shows a nucleotide sequence (SEQ ID NO:373) of a native sequence PRO83145 cDNA, wherein SEQ ID NO:373 is a clone designated herein as “DNA329132”.

FIG. 374 shows the amino acid sequence (SEQ ID NO:374) derived from the coding sequence of SEQ ID NO:373 shown in FIG. 373.

FIG. 375 shows a nucleotide sequence (SEQ ID NO:375) of a native sequence PRO84771 cDNA, wherein SEQ ID NO:375 is a clone designated herein as “DNA329133”.

FIG. 376 shows the amino acid sequence (SEQ ID NO:376) derived from the coding sequence of SEQ ID NO:375 shown in FIG. 375.

FIG. 377 shows a nucleotide sequence (SEQ ID NO:377) of a native sequence PRO83605 cDNA, wherein SEQ ID NO:377 is a clone designated herein as “DNA327585”.

FIG. 378 shows the amino acid sequence (SEQ ID NO:378) derived from the coding sequence of SEQ ID NO:377 shown in FIG. 377.

FIG. 379 shows a nucleotide sequence (SEQ ID NO:379) of a native sequence PRO71107 cDNA, wherein SEQ ID NO:379 is a clone designated herein as “DNA304681”.

FIG. 380 shows the amino acid sequence (SEQ ID NO:380) derived from the coding sequence of SEQ ID NO:379 shown in FIG. 379.

FIG. 381 shows a nucleotide sequence (SEQ ID NO:381) of a native sequence PRO59254 cDNA, wherein SEQ ID NO:381 is a clone designated herein as “DNA329134”.

FIG. 382 shows the amino acid sequence (SEQ ID NO:382) derived from the coding sequence of SEQ ID NO:381 shown in FIG. 381.

FIG. 383 shows a nucleotide sequence (SEQ ID NO:383) of a native sequence PRO84299 cDNA, wherein SEQ ID NO:383 is a clone designated herein as “DNA328473”.

FIG. 384 shows the amino acid sequence (SEQ ID NO:384) derived from the coding sequence of SEQ ID NO:383 shown in FIG. 383.

FIG. 385 shows a nucleotide sequence (SEQ ID NO:385) of a native sequence PRO37756 cDNA, wherein SEQ ID NO:385 is a clone designated herein as “DNA227293”.

FIG. 386 shows the amino acid sequence (SEQ ID NO:386) derived from the coding sequence of SEQ ID NO:385 shown in FIG. 385.

FIG. 387 shows a nucleotide sequence (SEQ ID NO:387) of a native sequence PRO58102 cDNA, wherein SEQ ID NO:387 is a clone designated herein as “DNA329135”.

FIG. 388 shows the amino acid sequence (SEQ ID NO:388) derived from the coding sequence of SEQ ID NO:387 shown in FIG. 387.

FIG. 389 shows a nucleotide sequence (SEQ ID NO:389) of a native sequence PRO37368 cDNA, wherein SEQ ID NO:389 is a clone designated herein as “DNA226905”.

FIG. 390 shows the amino acid sequence (SEQ ID NO:390) derived from the coding sequence of SEQ ID NO:389 shown in FIG. 389.

FIG. 391 shows a nucleotide sequence (SEQ ID NO:391) of a native sequence PRO84772 cDNA, wherein SEQ ID NO:391 is a clone designated herein as “DNA329136”.

FIG. 392 shows the amino acid sequence (SEQ ID NO:392) derived from the coding sequence of SEQ ID NO:391 shown in FIG. 391.

FIG. 393 shows a nucleotide sequence (SEQ ID NO:393) of a native sequence PRO12879 cDNA, wherein SEQ ID NO:393 is a clone designated herein as “DNA329137”.

FIG. 394 shows the amino acid sequence (SEQ ID NO:394) derived from the coding sequence of SEQ ID NO:393 shown in FIG. 393.

FIG. 395 shows a nucleotide sequence (SEQ ID NO:395) of a native sequence PRO38299 cDNA, wherein SEQ ID NO:395 is a clone designated herein as “DNA227836”.

FIG. 396 shows the amino acid sequence (SEQ ID NO:396) derived from the coding sequence of SEQ ID NO:395 shown in FIG. 395.

FIG. 397 shows a nucleotide sequence (SEQ ID NO:397) of a native sequence PRO84773 cDNA, wherein SEQ ID NO:397 is a clone designated herein as “DNA329138”.

FIG. 398 shows the amino acid sequence (SEQ ID NO:398) derived from the coding sequence of SEQ ID NO:397 shown in FIG. 397.

FIG. 399 shows a nucleotide sequence (SEQ ID NO:399) of a native sequence PRO84774 cDNA, wherein SEQ ID NO:399 is a clone designated herein as “DNA329139”.

FIG. 400 shows the amino acid sequence (SEQ ID NO:400) derived from the coding sequence of SEQ ID NO:399 shown in FIG. 399.

FIG. 401 shows a nucleotide sequence (SEQ ID NO:401) of a native sequence PRO60960 cDNA, wherein SEQ ID NO:401 is a clone designated herein as “DNA272867”.

FIG. 402 shows the amino acid sequence (SEQ ID NO:402) derived from the coding sequence of SEQ ID NO:401 shown in FIG. 401.

FIG. 403 shows a nucleotide sequence (SEQ ID NO:403) of a native sequence PRO12770 cDNA, wherein SEQ ID NO:403 is a clone designated herein as “DNA150430”.

FIG. 404 shows the amino acid sequence (SEQ ID NO:404) derived from the coding sequence of SEQ ID NO:403 shown in FIG. 403.

FIG. 405 shows a nucleotide sequence (SEQ ID NO:405) of a native sequence PRO71146 cDNA, wherein SEQ ID NO:405 is a clone designated herein as “DNA304720”.

FIG. 406 shows the amino acid sequence (SEQ ID NO:406) derived from the coding sequence of SEQ ID NO:405 shown in FIG. 405.

FIG. 407 shows a nucleotide sequence (SEQ ID NO:407) of a native sequence PRO60024 cDNA, wherein SEQ ID NO:407 is a clone designated herein as “DNA271740”.

FIG. 408 shows the amino acid sequence (SEQ ID NO:408) derived from the coding sequence of SEQ ID NO:407 shown in FIG. 407.

FIG. 409 shows a nucleotide sequence (SEQ ID NO:409) of a native sequence PRO60698 cDNA, wherein SEQ ID NO:409 is a clone designated herein as “DNA272449”.

FIG. 410 shows the amino acid sequence (SEQ ID NO:410) derived from the coding sequence of SEQ ID NO:409 shown in FIG. 409.

FIG. 411 shows a nucleotide sequence (SEQ ID NO:411) of a native sequence PRO84775 cDNA, wherein SEQ ID NO:411 is a clone designated herein as “DNA329140”.

FIG. 412 shows the amino acid sequence (SEQ ID NO:412) derived from the coding sequence of SEQ ID NO:411 shown in FIG. 411.

FIG. 413 shows a nucleotide sequence (SEQ ID NO:413) of a native sequence PRO59315 cDNA, wherein SEQ ID NO:413 is a clone designated herein as “DNA270985”.

FIG. 414 shows the amino acid sequence (SEQ ID NO:414) derived from the coding sequence of SEQ ID NO:413 shown in FIG. 413.

FIG. 415 shows a nucleotide sequence (SEQ ID NO:415) of a native sequence PRO80660 cDNA, wherein SEQ ID NO:415 is a clone designated herein as “DNA323927”.

FIG. 416 shows the amino acid sequence (SEQ ID NO:416) derived from the coding sequence of SEQ ID NO:415 shown in FIG. 415.

FIG. 417 shows a nucleotide sequence (SEQ ID NO:417) of a native sequence PRO51738 cDNA, wherein SEQ ID NO:417 is a clone designated herein as “DNA256807”.

FIG. 418 shows the amino acid sequence (SEQ ID NO:418) derived from the coding sequence of SEQ ID NO:417 shown in FIG. 417.

FIG. 419 shows a nucleotide sequence (SEQ ID NO:419) of a native sequence PRO84776 cDNA, wherein SEQ ID NO:419 is a clone designated herein as “DNA329141”.

FIG. 420 shows the amino acid sequence (SEQ ID NO:420) derived from the coding sequence of SEQ ID NO:419 shown in FIG. 419.

FIG. 421 shows a nucleotide sequence (SEQ ID NO:421) of a native sequence PRO84777 cDNA, wherein SEQ ID NO:421 is a clone designated herein as “DNA329142”.

FIG. 422 shows the amino acid sequence (SEQ ID NO:422) derived from the coding sequence of SEQ ID NO:421 shown in FIG. 421.

FIG. 423 shows a nucleotide sequence (SEQ ID NO:423) of a native sequence PRO60997 cDNA, wherein SEQ ID NO:423 is a clone designated herein as “DNA272911”.

FIG. 424 shows the amino acid sequence (SEQ ID NO:424) derived from the coding sequence of SEQ ID NO:423 shown in FIG. 423.

FIG. 425 shows a nucleotide sequence (SEQ ID NO:425) of a native sequence PRO84700 cDNA, wherein SEQ ID NO:425 is a clone designated herein as “DNA329033”.

FIG. 426 shows the amino acid sequence (SEQ ID NO:426) derived from the coding sequence of SEQ ID NO:425 shown in FIG. 425.

FIG. 427 shows a nucleotide sequence (SEQ ID NO:427) of a native sequence PRO84778 cDNA, wherein SEQ ID NO:427 is a clone designated herein as “DNA329143”.

FIG. 428 shows the amino acid sequence (SEQ ID NO:428) derived from the coding sequence of SEQ ID NO:427 shown in FIG. 427.

FIG. 429 shows a nucleotide sequence (SEQ ID NO:429) of a native sequence PRO69521 cDNA, wherein SEQ ID NO:429 is a clone designated herein as “DNA287246”.

FIG. 430 shows the amino acid sequence (SEQ ID NO:430) derived from the coding sequence of SEQ ID NO:429 shown in FIG. 429.

FIG. 431 shows a nucleotide sequence (SEQ ID NO:431) of a native sequence PRO84779 cDNA, wherein SEQ ID NO:431 is a clone designated herein as “DNA329144”.

FIG. 432 shows the amino acid sequence (SEQ ID NO:432) derived from the coding sequence of SEQ ID NO:431 shown in FIG. 431.

FIG. 433 shows a nucleotide sequence (SEQ ID NO:433) of a native sequence PRO80881 cDNA, wherein SEQ ID NO:433 is a clone designated herein as “DNA324183”.

FIG. 434 shows the amino acid sequence (SEQ ID NO:434) derived from the coding sequence of SEQ ID NO:433 shown in FIG. 433.

FIG. 435 shows a nucleotide sequence (SEQ ID NO:435) of a native sequence PRO37791 cDNA, wherein SEQ ID NO:435 is a clone designated herein as “DNA326322”.

FIG. 436 shows the amino acid sequence (SEQ ID NO:436) derived from the coding sequence of SEQ ID NO:435 shown in FIG. 435.

FIG. 437 shows a nucleotide sequence (SEQ ID NO:437) of a native sequence PRO37815 cDNA, wherein SEQ ID NO:437 is a clone designated herein as “DNA328513”.

FIG. 438 shows the amino acid sequence (SEQ ID NO:438) derived from the coding sequence of SEQ ID NO:437 shown in FIG. 437.

FIG. 439 shows a nucleotide sequence (SEQ ID NO:439) of a native sequence PRO1723 cDNA, wherein SEQ ID NO:439 is a clone designated herein as “DNA82376”.

FIG. 440 shows the amino acid sequence (SEQ ID NO:440) derived from the coding sequence of SEQ ID NO:439 shown in FIG. 439.

FIG. 441 shows a nucleotide sequence (SEQ ID NO:441) of a native sequence PRO2711 cDNA, wherein SEQ ID NO:441 is a clone designated herein as “DNA88239”.

FIG. 442 shows the amino acid sequence (SEQ ID NO:442) derived from the coding sequence of SEQ ID NO:441 shown in FIG. 441.

FIG. 443 shows a nucleotide sequence (SEQ ID NO:443) of a native sequence PRO36378 cDNA, wherein SEQ ID NO:443 is a clone designated herein as “DNA225915”.

FIG. 444 shows the amino acid sequence (SEQ ID NO:444) derived from the coding sequence of SEQ ID NO:443 shown in FIG. 443.

FIG. 445 shows a nucleotide sequence (SEQ ID NO:445) of a native sequence PRO84780 cDNA, wherein SEQ ID NO:445 is a clone designated herein as “DNA329145”.

FIG. 446 shows the amino acid sequence (SEQ ID NO:446) derived from the coding sequence of SEQ ID NO:445 shown in FIG. 445.

FIG. 447 shows a nucleotide sequence (SEQ ID NO:447) of a native sequence PRO70001 cDNA, wherein SEQ ID NO:447 is a clone designated herein as “DNA329146”.

FIG. 448 shows the amino acid sequence (SEQ ID NO:448) derived from the coding sequence of SEQ ID NO:447 shown in FIG. 447.

FIG. 449 shows a nucleotide sequence (SEQ ID NO:449) of a native sequence PRO37200 cDNA, wherein SEQ ID NO:449 is a clone designated herein as “DNA226737”.

FIG. 450 shows the amino acid sequence (SEQ ID NO:450) derived from the coding sequence of SEQ ID NO:449 shown in FIG. 449.

FIG. 451 shows a nucleotide sequence (SEQ ID NO:451) of a native sequence PRO84781 cDNA, wherein SEQ ID NO:451 is a clone designated herein as “DNA329147”.

FIG. 452 shows the amino acid sequence (SEQ ID NO:452) derived from the coding sequence of SEQ ID NO:451 shown in FIG. 451.

FIG. 453 shows a nucleotide sequence (SEQ ID NO:453) of a native sequence PRO34276 cDNA, wherein SEQ ID NO:453 is a clone designated herein as “DNA216689”.

FIG. 454 shows the amino acid sequence (SEQ ID NO:454) derived from the coding sequence of SEQ ID NO:453 shown in FIG. 453.

FIG. 455 shows a nucleotide sequence (SEQ ID NO:455) of a native sequence PRO12313 cDNA, wherein SEQ ID NO:455 is a clone designated herein as “DNA150529”.

FIG. 456 shows the amino acid sequence (SEQ ID NO:456) derived from the coding sequence of SEQ ID NO:455 shown in FIG. 455.

FIG. 457 shows a nucleotide sequence (SEQ ID NO:457) of a native sequence PRO61870 cDNA, wherein SEQ ID NO:457 is a clone designated herein as “DNA273919”.

FIG. 458 shows the amino acid sequence (SEQ ID NO:458) derived from the coding sequence of SEQ ID NO:457 shown in FIG. 457.

FIG. 459 shows a nucleotide sequence (SEQ ID NO:459) of a native sequence PRO37579 cDNA, wherein SEQ ID NO:459 is a clone designated herein as “DNA227116”.

FIG. 460 shows the amino acid sequence (SEQ ID NO:460) derived from the coding sequence of SEQ ID NO:459 shown in FIG. 459.

FIG. 461 shows a nucleotide sequence (SEQ ID NO:461) of a native sequence PRO60781 cDNA, wherein SEQ ID NO:461 is a clone designated herein as “DNA272655”.

FIG. 462 shows the amino acid sequence (SEQ ID NO:462) derived from the coding sequence of SEQ ID NO:461 shown in FIG. 461.

FIG. 463 shows a nucleotide sequence (SEQ ID NO:463) of a native sequence PRO84782 cDNA, wherein SEQ ID NO:463 is a clone designated herein as “DNA329148”.

FIG. 464 shows the amino acid sequence (SEQ ID NO:464) derived from the coding sequence of SEQ ID NO:463 shown in FIG. 463.

FIG. 465 shows a nucleotide sequence (SEQ ID NO:465) of a native sequence PRO12481 cDNA, wherein SEQ ID NO:465 is a clone designated herein as “DNA150812”.

FIG. 466 shows the amino acid sequence (SEQ ID NO:466) derived from the coding sequence of SEQ ID NO:465 shown in FIG. 465.

FIG. 467 shows a nucleotide sequence (SEQ ID NO:467) of a native sequence PRO4854 cDNA, wherein SEQ ID NO:467 is a clone designated herein as “DNA103527”.

FIG. 468 shows the amino acid sequence (SEQ ID NO:468) derived from the coding sequence of SEQ ID NO:467 shown in FIG. 467.

FIG. 469 shows a nucleotide sequence (SEQ ID NO:469) of a native sequence PRO37040 cDNA, wherein SEQ ID NO:469 is a clone designated herein as “DNA226577”.

FIG. 470 shows the amino acid sequence (SEQ ID NO:470) derived from the coding sequence of SEQ ID NO:469 shown in FIG. 469.

FIG. 471 shows a nucleotide sequence (SEQ ID NO:471) of a native sequence PRO61763 cDNA, wherein SEQ ID NO:471 is a clone designated herein as “DNA273802”.

FIG. 472 shows the amino acid sequence (SEQ ID NO:472) derived from the coding sequence of SEQ ID NO:471 shown in FIG. 471.

FIG. 473 shows a nucleotide sequence (SEQ ID NO:473) of a native sequence PRO20111 cDNA, wherein SEQ ID NO:473 is a clone designated herein as “DNA329149”.

FIG. 474 shows the amino acid sequence (SEQ ID NO:474) derived from the coding sequence of SEQ ID NO:473 shown in FIG. 473.

FIG. 475 shows a nucleotide sequence (SEQ ID NO:475) of a native sequence PRO4793 cDNA, wherein SEQ ID NO:475 is a clone designated herein as “DNA325800”.

FIG. 476 shows the amino acid sequence (SEQ ID NO:476) derived from the coding sequence of SEQ ID NO:475 shown in FIG. 475.

FIG. 477 shows a nucleotide sequence (SEQ ID NO:477) of a native sequence PRO84783 cDNA, wherein SEQ ID NO:477 is a clone designated herein as “DNA329150”.

FIG. 478 shows the amino acid sequence (SEQ ID NO:478) derived from the coding sequence of SEQ ID NO:477 shown in FIG. 477.

FIG. 479 shows a nucleotide sequence (SEQ ID NO:479) of a native sequence PRO84703 cDNA, wherein SEQ ID NO:479 is a clone designated herein as “DNA329036”.

FIG. 480 shows the amino acid sequence (SEQ ID NO:480) derived from the coding sequence of SEQ ID NO:479 shown in FIG. 479.

FIG. 481 shows a nucleotide sequence (SEQ ID NO:481) of a native sequence PRO12173 cDNA, wherein SEQ ID NO:481 is a clone designated herein as “DNA151067”.

FIG. 482 shows the amino acid sequence (SEQ ID NO:482) derived from the coding sequence of SEQ ID NO:481 shown in FIG. 481.

FIG. 483 shows a nucleotide sequence (SEQ ID NO:483) of a native sequence PRO36542 cDNA, wherein SEQ ID NO:483 is a clone designated herein as “DNA226079”.

FIG. 484 shows the amino acid sequence (SEQ ID NO:484) derived from the coding sequence of SEQ ID NO:483 shown in FIG. 483.

FIG. 485 shows a nucleotide sequence (SEQ ID NO:485) of a native sequence PRO37560 cDNA, wherein SEQ ID NO:485 is a clone designated herein as “DNA227097”.

FIG. 486 shows the amino acid sequence (SEQ ID NO:486) derived from the coding sequence of SEQ ID NO:485 shown in FIG. 485.

FIG. 487 shows a nucleotide sequence (SEQ ID NO:487) of a native sequence PRO84784 cDNA, wherein SEQ ID NO:487 is a clone designated herein as “DNA329151”.

FIG. 488 shows the amino acid sequence (SEQ ID NO:488) derived from the coding sequence of SEQ ID NO:487 shown in FIG. 487.

FIG. 489 shows a nucleotide sequence (SEQ ID NO:489) of a native sequence PRO84785 cDNA, wherein SEQ ID NO:489 is a clone designated herein as “DNA329152”.

FIG. 490 shows the amino acid sequence (SEQ ID NO:490) derived from the coding sequence of SEQ ID NO:489 shown in FIG. 489.

FIG. 491 shows a nucleotide sequence (SEQ ID NO:491) of a native sequence PRO81753 cDNA, wherein SEQ ID NO:491 is a clone designated herein as “DNA325192”.

FIG. 492 shows the amino acid sequence (SEQ ID NO:492) derived from the coding sequence of SEQ ID NO:491 shown in FIG. 491.

FIG. 493 shows a nucleotide sequence (SEQ ID NO:493) of a native sequence PRO84786 cDNA, wherein SEQ ID NO:493 is a clone designated herein as “DNA329153”.

FIG. 494 shows the amino acid sequence (SEQ ID NO:494) derived from the coding sequence of SEQ ID NO:493 shown in FIG. 493.

FIG. 495 shows a nucleotide sequence (SEQ ID NO:495) of a native sequence PRO69568 cDNA, wherein SEQ ID NO:495 is a clone designated herein as “DNA329154”.

FIG. 496 shows the amino acid sequence (SEQ ID NO:496) derived from the coding sequence of SEQ ID NO:495 shown in FIG. 495.

FIG. 497 shows a nucleotide sequence (SEQ ID NO:497) of a native sequence PRO1207 cDNA, wherein SEQ ID NO:497 is a clone designated herein as “DNA329155”.

FIG. 498 shows the amino acid sequence (SEQ ID NO:498) derived from the coding sequence of SEQ ID NO:497 shown in FIG. 497.

FIG. 499 shows a nucleotide sequence (SEQ ID NO:499) of a native sequence PRO84787 cDNA, wherein SEQ ID NO:499 is a clone designated herein as “DNA329156”.

FIG. 500 shows the amino acid sequence (SEQ ID NO:500) derived from the coding sequence of SEQ ID NO:499 shown in FIG. 499.

FIG. 501 shows a nucleotide sequence (SEQ ID NO:501) of a native sequence PRO49183 cDNA, wherein SEQ ID NO:501 is a clone designated herein as “DNA253585”.

FIG. 502 shows the amino acid sequence (SEQ ID NO:502) derived from the coding sequence of SEQ ID NO:501 shown in FIG. 501.

FIG. 503 shows a nucleotide sequence (SEQ ID NO:503) of a native sequence PRO60670 cDNA, wherein SEQ ID NO:503 is a clone designated herein as “DNA272417”.

FIG. 504 shows the amino acid sequence (SEQ ID NO:504) derived from the coding sequence of SEQ ID NO:503 shown in FIG. 503.

FIG. 505 shows a nucleotide sequence (SEQ ID NO:505) of a native sequence PRO62861 cDNA, wherein SEQ ID NO:505 is a clone designated herein as “DNA329157”.

FIG. 506 shows the amino acid sequence (SEQ ID NO:506) derived from the coding sequence of SEQ ID NO:505 shown in FIG. 505.

FIG. 507 shows a nucleotide sequence (SEQ ID NO:507) of a native sequence PRO2536 cDNA, wherein SEQ ID NO:507 is a clone designated herein as “DNA76503”.

FIG. 508 shows the amino acid sequence (SEQ ID NO:508) derived from the coding sequence of SEQ ID NO:507 shown in FIG. 507.

FIG. 509 shows a nucleotide sequence (SEQ ID NO:509) of a native sequence PRO38477 cDNA, wherein SEQ ID NO:509 is a clone designated herein as “DNA228014”.

FIG. 510 shows the amino acid sequence (SEQ ID NO:510) derived from the coding sequence of SEQ ID NO:509 shown in FIG. 509.

FIG. 511 shows a nucleotide sequence (SEQ ID NO:511) of a native sequence PRO12243 cDNA, wherein SEQ ID NO:511 is a clone designated herein as “DNA150427”.

FIG. 512 shows the amino acid sequence (SEQ ID NO:512) derived from the coding sequence of SEQ ID NO:511 shown in FIG. 511.

FIG. 513 shows a nucleotide sequence (SEQ ID NO:513) of a native sequence PRO84788 cDNA, wherein SEQ ID NO:513 is a clone designated herein as “DNA329158”.

FIG. 514 shows the amino acid sequence (SEQ ID NO:514) derived from the coding sequence of SEQ ID NO:513 shown in FIG. 513.

FIG. 515 shows a nucleotide sequence (SEQ ID NO:515) of a native sequence PRO4660 cDNA, wherein SEQ ID NO:515 is a clone designated herein as “DNA329159”.

FIG. 516 shows the amino acid sequence (SEQ ID NO:516) derived from the coding sequence of SEQ ID NO:515 shown in FIG. 515.

FIG. 517 shows a nucleotide sequence (SEQ ID NO:517) of a native sequence PRO81585 cDNA, wherein SEQ ID NO:517 is a clone designated herein as “DNA324991”.

FIG. 518 shows the amino acid sequence (SEQ ID NO:518) derived from the coding sequence of SEQ ID NO:517 shown in FIG. 517.

FIG. 519 shows a nucleotide sequence (SEQ ID NO:519) of a native sequence PRO83635 cDNA, wherein SEQ ID NO:519 is a clone designated herein as “DNA327632”.

FIG. 520 shows the amino acid sequence (SEQ ID NO:520) derived from the coding sequence of SEQ ID NO:519 shown in Figure.

FIG. 521 shows a nucleotide sequence (SEQ ID NO:521) of a native sequence PRO21960 cDNA, wherein SEQ ID NO:521 is a clone designated herein as “DNA192060”.

FIG. 522 shows the amino acid sequence (SEQ ID NO:522) derived from the coding sequence of SEQ ID NO:521 shown in FIG. 521.

FIG. 523 shows a nucleotide sequence (SEQ ID NO:523) of a native sequence PRO62760 cDNA, wherein SEQ ID NO:523 is a clone designated herein as “DNA299899”.

FIG. 524 shows the amino acid sequence (SEQ ID NO:524) derived from the coding sequence of SEQ ID NO:523 shown in FIG. 523.

FIG. 525 shows a nucleotide sequence (SEQ ID NO:525) of a native sequence PRO84789 cDNA, wherein SEQ ID NO:525 is a clone designated herein as “DNA329160”.

FIG. 526 shows the amino acid sequence (SEQ ID NO:526) derived from the coding sequence of SEQ ID NO:525 shown in FIG. 525.

FIG. 527 shows a nucleotide sequence (SEQ ID NO:527) of a native sequence PRO21708 cDNA, wherein SEQ ID NO:527 is a clone designated herein as “DNA188333”.

FIG. 528 shows the amino acid sequence (SEQ ID NO:528) derived from the coding sequence of SEQ ID NO:527 shown in FIG. 527.

FIG. 529 shows a nucleotide sequence (SEQ ID NO:529) of a native sequence PRO37544 cDNA, wherein SEQ ID NO:529 is a clone designated herein as “DNA227081”.

FIG. 530 shows the amino acid sequence (SEQ ID NO:530) derived from the coding sequence of SEQ ID NO:529 shown in Figure.

FIG. 531 shows a nucleotide sequence (SEQ ID NO:531) of a native sequence PRO37910 cDNA, wherein SEQ ID NO:531 is a clone designated herein as “DNA227447”.

FIG. 532 shows the amino acid sequence (SEQ ID NO:532) derived from the coding sequence of SEQ ID NO:531 shown in FIG. 531.

FIG. 533 shows a nucleotide sequence (SEQ ID NO:533) of a native sequence PRO21834 cDNA, wherein SEQ ID NO:533 is a clone designated herein as “DNA188301”.

FIG. 534 shows the amino acid sequence (SEQ ID NO:534) derived from the coding sequence of SEQ ID NO:533 shown in FIG. 533.

FIG. 535 shows a nucleotide sequence (SEQ ID NO:535) of a native sequence PRO37636 cDNA, wherein SEQ ID NO:535 is a clone designated herein as “DNA227173”.

FIG. 536 shows the amino acid sequence (SEQ ID NO:536) derived from the coding sequence of SEQ ID NO:535 shown in FIG. 535.

FIG. 537 shows a nucleotide sequence (SEQ ID NO:537) of a native sequence PRO84790 cDNA, wherein SEQ ID NO:537 is a clone designated herein as “DNA329161”.

FIG. 538 shows the amino acid sequence (SEQ ID NO:538) derived from the coding sequence of SEQ ID NO:537 shown in FIG. 537.

FIG. 539 shows a nucleotide sequence (SEQ ID NO:539) of a native sequence PRO21885 cDNA, wherein SEQ ID NO:539 is a clone designated herein as “DNA188355”.

FIG. 540 shows the amino acid sequence (SEQ ID NO:540) derived from the coding sequence of SEQ ID NO:539 shown in FIG. 539.

FIG. 541 shows a nucleotide sequence (SEQ ID NO:541) of a native sequence PRO51301 cDNA, wherein SEQ ID NO:541 is a clone designated herein as “DNA256257”.

FIG. 542 shows the amino acid sequence (SEQ ID NO:542) derived from the coding sequence of SEQ ID NO:541 shown in FIG. 541.

FIG. 543 shows a nucleotide sequence (SEQ ID NO:543) of a native sequence PRO60077 cDNA, wherein SEQ ID NO:543 is a clone designated herein as “DNA329162”.

FIG. 544 shows the amino acid sequence (SEQ ID NO:544) derived from the coding sequence of SEQ ID NO:543 shown in FIG. 543.

FIG. 545 shows a nucleotide sequence (SEQ ID NO:545) of a native sequence PRO83644 cDNA, wherein SEQ ID NO:545 is a clone designated herein as “DNA327643”.

FIG. 546 shows the amino acid sequence (SEQ ID NO:546) derived from the coding sequence of SEQ ID NO:545 shown in FIG. 545.

FIG. 547 shows a nucleotide sequence (SEQ ID NO:547) of a native sequence cDNA, wherein SEQ ID NO:547 is a clone designated herein as “DNA329163”.

FIG. 548 shows a nucleotide sequence (SEQ ID NO:548) of a native sequence PRO84792 cDNA, wherein SEQ ID NO:548 is a clone designated herein as “DNA329164”.

FIG. 549 shows the amino acid sequence (SEQ ID NO:549) derived from the coding sequence of SEQ ID NO:548 shown in FIG. 548.

FIG. 550 shows a nucleotide sequence (SEQ ID NO:550) of a native sequence PRO81000 cDNA, wherein SEQ ID NO:550 is a clone designated herein as “DNA324324”.

FIG. 551 shows the amino acid sequence (SEQ ID NO:551) derived from the coding sequence of SEQ ID NO:550 shown in FIG. 550.

FIG. 552 shows a nucleotide sequence (SEQ ID NO:552) of a native sequence PRO37843 cDNA, wherein SEQ ID NO:552 is a clone designated herein as “DNA328570”.

FIG. 553 shows the amino acid sequence (SEQ ID NO:553) derived from the coding sequence of SEQ ID NO:552 shown in FIG. 552.

FIG. 554 shows a nucleotide sequence (SEQ ID NO:554) of a native sequence PRO63054 cDNA, wherein SEQ ID NO:554 is a clone designated herein as “DNA329165”.

FIG. 555 shows the amino acid sequence (SEQ ID NO:555) derived from the coding sequence of SEQ ID NO:554 shown in FIG. 554.

FIG. 556 shows a nucleotide sequence (SEQ ID NO:556) of a native sequence PRO12374 cDNA, wherein SEQ ID NO:556 is a clone designated herein as “DNA150621”.

FIG. 557 shows the amino acid sequence (SEQ ID NO:557) derived from the coding sequence of SEQ ID NO:556 shown in FIG. 556.

FIG. 558 shows a nucleotide sequence (SEQ ID NO:558) of a native sequence PRO2541 cDNA, wherein SEQ ID NO:558 is a clone designated herein as “DNA76517”.

FIG. 559 shows the amino acid sequence (SEQ ID NO:559) derived from the coding sequence of SEQ ID NO:558 shown in FIG. 558.

FIG. 560 shows a nucleotide sequence (SEQ ID NO:560) of a native sequence PRO4940 cDNA, wherein SEQ ID NO:560 is a clone designated herein as “DNA328576”.

FIG. 561 shows the amino acid sequence (SEQ ID NO:561) derived from the coding sequence of SEQ ID NO:560 shown in FIG. 560.

FIG. 562 shows a nucleotide sequence (SEQ ID NO:562) of a native sequence PRO2524 cDNA, wherein SEQ ID NO:562 is a clone designated herein as “DNA75525”.

FIG. 563 shows the amino acid sequence (SEQ ID NO:563) derived from the coding sequence of SEQ ID NO:562 shown in FIG. 562.

FIG. 564 shows a nucleotide sequence (SEQ ID NO:564) of a native sequence PRO59203 cDNA, wherein SEQ ID NO:564 is a clone designated herein as “DNA270867”.

FIG. 565 shows the amino acid sequence (SEQ ID NO:565) derived from the coding sequence of SEQ ID NO:564 shown in FIG. 564.

FIG. 566 shows a nucleotide sequence (SEQ ID NO:566) of a native sequence PRO2022 cDNA, wherein SEQ ID NO:566 is a clone designated herein as “DNA76516”.

FIG. 567 shows the amino acid sequence (SEQ ID NO:567) derived from the coding sequence of SEQ ID NO:566 shown in FIG. 566.

FIG. 568 shows a nucleotide sequence (SEQ ID NO:568) of a native sequence PRO58425 cDNA, wherein SEQ ID NO:568 is a clone designated herein as “DNA329047”.

FIG. 569 shows the amino acid sequence (SEQ ID NO:569) derived from the coding sequence of SEQ ID NO:568 shown in FIG. 568.

FIG. 570 shows a nucleotide sequence (SEQ ID NO:570) of a native sequence PRO4611 cDNA, wherein SEQ ID NO:570 is a clone designated herein as “DNA103281”.

FIG. 571 shows the amino acid sequence (SEQ ID NO:571) derived from the coding sequence of SEQ ID NO:570 shown in FIG. 570.

FIG. 572 shows a nucleotide sequence (SEQ ID NO:572) of a native sequence PRO84793 cDNA, wherein SEQ ID NO:572 is a clone designated herein as “DNA329166”.

FIG. 573 shows the amino acid sequence (SEQ ID NO:573) derived from the coding sequence of SEQ ID NO:572 shown in FIG. 572.

FIG. 574 shows a nucleotide sequence (SEQ ID NO:574) of a native sequence PRO2068 cDNA, wherein SEQ ID NO:574 is a clone designated herein as “DNA83063”.

FIG. 575 shows the amino acid sequence (SEQ ID NO:575) derived from the coding sequence of SEQ ID NO:574 shown in FIG. 574.

FIG. 576 shows a nucleotide sequence (SEQ ID NO:576) of a native sequence PRO12876 cDNA, wherein SEQ ID NO:576 is a clone designated herein as “DNA151420”.

FIG. 577 shows the amino acid sequence (SEQ ID NO:577) derived from the coding sequence of SEQ ID NO:576 shown in FIG. 576.

FIG. 578 shows a nucleotide sequence (SEQ ID NO:578) of a native sequence PRO38147 cDNA, wherein SEQ ID NO:578 is a clone designated herein as “DNA327657”.

FIG. 579 shows the amino acid sequence (SEQ ID NO:579) derived from the coding sequence of SEQ ID NO:578 shown in FIG. 578.

FIG. 580 shows a nucleotide sequence (SEQ ID NO:580) of a native sequence PRO4933 cDNA, wherein SEQ ID NO:580 is a clone designated herein as “DNA329167”.

FIG. 581 shows the amino acid sequence (SEQ ID NO:581) derived from the coding sequence of SEQ ID NO:580 shown in FIG. 580.

FIG. 582 shows a nucleotide sequence (SEQ ID NO:582) of a native sequence PRO12612 cDNA, wherein SEQ ID NO:582 is a clone designated herein as “DNA329005”.

FIG. 583 shows the amino acid sequence (SEQ ID NO:583) derived from the coding sequence of SEQ ID NO:582 shown in FIG. 582.

FIG. 584 shows a nucleotide sequence (SEQ ID NO:584) of a native sequence PRO84794 cDNA, wherein SEQ ID NO:584 is a clone designated herein as “DNA329168”.

FIG. 585 shows the amino acid sequence (SEQ ID NO:585) derived from the coding sequence of SEQ ID NO:584 shown in FIG. 584.

FIG. 586 shows a nucleotide sequence (SEQ ID NO:586) of a native sequence PRO36521 cDNA, wherein SEQ ID NO:586 is a clone designated herein as “DNA226058”.

FIG. 587 shows the amino acid sequence (SEQ ID NO:587) derived from the coding sequence of SEQ ID NO:586 shown in FIG. 586.

FIG. 588 shows a nucleotide sequence (SEQ ID NO:588) of a native sequence PRO34330 cDNA, wherein SEQ ID NO:588 is a clone designated herein as “DNA218278”.

FIG. 589 shows the amino acid sequence (SEQ ID NO:589) derived from the coding sequence of SEQ ID NO:588 shown in FIG. 588.

FIG. 590 shows a nucleotide sequence (SEQ ID NO:590) of a native sequence PRO37671 cDNA, wherein SEQ ID NO:590 is a clone designated herein as “DNA227208”.

FIG. 591 shows the amino acid sequence (SEQ ID NO:591) derived from the coding sequence of SEQ ID NO:590 shown in FIG. 590.

FIG. 592 shows a nucleotide sequence (SEQ ID NO:592) of a native sequence PRO1610 cDNA, wherein SEQ ID NO:592 is a clone designated herein as “DNA329169”.

FIG. 593 shows the amino acid sequence (SEQ ID NO:593) derived from the coding sequence of SEQ ID NO:592 shown in FIG. 592.

FIG. 594 shows a nucleotide sequence (SEQ ID NO:594) of a native sequence PRO24922 cDNA, wherein SEQ ID NO:594 is a clone designated herein as “DNA196424”.

FIG. 595 shows the amino acid sequence (SEQ ID NO:595) derived from the coding sequence of SEQ ID NO:594 shown in FIG. 594.

FIG. 596 shows a nucleotide sequence (SEQ ID NO:596) of a native sequence PRO83069 cDNA, wherein SEQ ID NO:596 is a clone designated herein as “DNA326727”.

FIG. 597 shows the amino acid sequence (SEQ ID NO:597) derived from the coding sequence of SEQ ID NO:596 shown in FIG. 596.

FIG. 598 shows a nucleotide sequence (SEQ ID NO:598) of a native sequence PRO70938 cDNA, wherein SEQ ID NO:598 is a clone designated herein as “DNA329170”.

FIG. 599 shows the amino acid sequence (SEQ ID NO:599) derived from the coding sequence of SEQ ID NO:598 shown in FIG. 598.

FIG. 600 shows a nucleotide sequence (SEQ ID NO:600) of a native sequence PRO84795 cDNA, wherein SEQ ID NO:600 is a clone designated herein as “DNA329171”.

FIG. 601 shows the amino acid sequence (SEQ ID NO:601) derived from the coding sequence of SEQ ID NO:600 shown in FIG. 600.

FIG. 602 shows a nucleotide sequence (SEQ ID NO:602) of a native sequence PRO84796 cDNA, wherein SEQ ID NO:602 is a clone designated herein as “DNA329172”.

FIG. 603 shows the amino acid sequence (SEQ ID NO:603) derived from the coding sequence of SEQ ID NO:602 shown in FIG. 602.

FIG. 604 shows a nucleotide sequence (SEQ ID NO:604) of a native sequence PRO83141 cDNA, wherein SEQ ID NO:604 is a clone designated herein as “DNA329173”.

FIG. 605 shows the amino acid sequence (SEQ ID NO:605) derived from the coding sequence of SEQ ID NO:604 shown in FIG. 604.

FIG. 606 shows a nucleotide sequence (SEQ ID NO:606) of a native sequence PRO2768 cDNA, wherein SEQ ID NO:606 is a clone designated herein as “DNA88374”.

FIG. 607 shows the amino acid sequence (SEQ ID NO:607) derived from the coding sequence of SEQ ID NO:606 shown in FIG. 606.

FIG. 608 shows a nucleotide sequence (SEQ ID NO:608) of a native sequence PRO84797 cDNA, wherein SEQ ID NO:608 is a clone designated herein as “DNA329174”.

FIG. 609 shows the amino acid sequence (SEQ ID NO:609) derived from the coding sequence of SEQ ID NO:608 shown in FIG. 608.

FIG. 610 shows a nucleotide sequence (SEQ ID NO:610) of a native sequence PRO49572 cDNA, wherein SEQ ID NO:610 is a clone designated herein as “DNA254464”.

FIG. 611 shows the amino acid sequence (SEQ ID NO:611) derived from the coding sequence of SEQ ID NO:610 shown in FIG. 610.

FIG. 612 shows a nucleotide sequence (SEQ ID NO:612) of a native sequence PRO2693 cDNA, wherein SEQ ID NO:612 is a clone designated herein as “DNA88195”.

FIG. 613 shows the amino acid sequence (SEQ ID NO:613) derived from the coding sequence of SEQ ID NO:612 shown in FIG. 612.

FIG. 614 shows a nucleotide sequence (SEQ ID NO:614) of a native sequence PRO60433 cDNA, wherein SEQ ID NO:614 is a clone designated herein as “DNA272165”.

FIG. 615 shows the amino acid sequence (SEQ ID NO:615) derived from the coding sequence of SEQ ID NO:614 shown in FIG. 614.

FIG. 616 shows a nucleotide sequence (SEQ ID NO:616) of a native sequence PRO51592 cDNA, wherein SEQ ID NO:616 is a clone designated herein as “DNA256561”.

FIG. 617 shows the amino acid sequence (SEQ ID NO:617) derived from the coding sequence of SEQ ID NO:616 shown in FIG. 616.

FIG. 618 shows a nucleotide sequence (SEQ ID NO:618) of a native sequence PRO84798 cDNA, wherein SEQ ID NO:618 is a clone designated herein as “DNA329175”.

FIG. 619 shows the amino acid sequence (SEQ ID NO:619) derived from the coding sequence of SEQ ID NO:618 shown in FIG. 618.

FIG. 620 shows a nucleotide sequence (SEQ ID NO:620) of a native sequence PRO7 cDNA, wherein SEQ ID NO: is a clone designated herein as “DNA35629”.

FIG. 621 shows the amino acid sequence (SEQ ID NO:621) derived from the coding sequence of SEQ ID NO:620 shown in FIG. 620.

FIG. 622 shows a nucleotide sequence (SEQ ID NO:622) of a native sequence PRO84376 cDNA, wherein SEQ ID NO:622 is a clone designated herein as “DNA328591”.

FIG. 623 shows the amino acid sequence (SEQ ID NO:623) derived from the coding sequence of SEQ ID NO:622 shown in FIG. 622.

FIG. 624 shows a nucleotide sequence (SEQ ID NO:624) of a native sequence PRO38213 cDNA, wherein SEQ ID NO:624 is a clone designated herein as “DNA227750”.

FIG. 625 shows the amino acid sequence (SEQ ID NO:625) derived from the coding sequence of SEQ ID NO:624 shown in FIG. 624.

FIG. 626 shows a nucleotide sequence (SEQ ID NO:626) of a native sequence PRO61201 cDNA, wherein SEQ ID NO:626 is a clone designated herein as “DNA273159”.

FIG. 627 shows the amino acid sequence (SEQ ID NO:627) derived from the coding sequence of SEQ ID NO:626 shown in FIG. 626.

FIG. 628 shows a nucleotide sequence (SEQ ID NO:628) of a native sequence PRO84799 cDNA, wherein SEQ ID NO:628 is a clone designated herein as “DNA329176”.

FIG. 629 shows the amino acid sequence (SEQ ID NO:629) derived from the coding sequence of SEQ ID NO:628 shown in FIG. 628.

FIG. 630 shows a nucleotide sequence (SEQ ID NO:630) of a native sequence PRO34451 cDNA, wherein SEQ ID NO:630 is a clone designated herein as “DNA218655”.

FIG. 631 shows the amino acid sequence (SEQ ID NO:631) derived from the coding sequence of SEQ ID NO:630 shown in FIG. 630.

FIG. 632 shows a nucleotide sequence (SEQ ID NO:632) of a native sequence PRO83661 cDNA, wherein SEQ ID NO:632 is a clone designated herein as “DNA327674”.

FIG. 633 shows the amino acid sequence (SEQ ID NO:633) derived from the coding sequence of SEQ ID NO:632 shown in FIG. 632.

FIG. 634 shows a nucleotide sequence (SEQ ID NO:634) of a native sequence PRO84800 cDNA, wherein SEQ ID NO:634 is a clone designated herein as “DNA329177”.

FIG. 635 shows the amino acid sequence (SEQ ID NO:635) derived from the coding sequence of SEQ ID NO:634 shown in FIG. 634.

FIG. 636 shows a nucleotide sequence (SEQ ID NO:636) of a native sequence PRO38214 cDNA, wherein SEQ ID NO:636 is a clone designated herein as “DNA227751”.

FIG. 637 shows the amino acid sequence (SEQ ID NO:637) derived from the coding sequence of SEQ ID NO:636 shown in FIG. 636.

FIG. 638 shows a nucleotide sequence (SEQ ID NO:638) of a native sequence PRO36999 cDNA, wherein SEQ ID NO:638 is a clone designated herein as “DNA226536”.

FIG. 639 shows the amino acid sequence (SEQ ID NO:639) derived from the coding sequence of SEQ ID NO:638 shown in FIG. 638.

FIG. 640 shows a nucleotide sequence (SEQ ID NO:640) of a native sequence PRO36859 cDNA, wherein SEQ ID NO:640 is a clone designated herein as “DNA226396”.

FIG. 641 shows the amino acid sequence (SEQ ID NO:641) derived from the coding sequence of SEQ ID NO:640 shown in FIG. 640.

FIG. 642 shows a nucleotide sequence (SEQ ID NO:642) of a native sequence PRO73 cDNA, wherein SEQ ID NO:642 is a clone designated herein as “DNA36718”.

FIG. 643 shows the amino acid sequence (SEQ ID NO:643) derived from the coding sequence of SEQ ID NO:642 shown in FIG. 642.

FIG. 644 shows a nucleotide sequence (SEQ ID NO:644) of a native sequence PRO848001 cDNA, wherein SEQ ID NO:644 is a clone designated herein as “DNA329178”.

FIG. 645 shows the amino acid sequence (SEQ ID NO:645) derived from the coding sequence of SEQ ID NO:644 shown in FIG. 644.

FIG. 646 shows a nucleotide sequence (SEQ ID NO:646) of a native sequence PRO84381 cDNA, wherein SEQ ID NO:646 is a clone designated herein as “DNA328597”.

FIG. 647 shows the amino acid sequence (SEQ ID NO:647) derived from the coding sequence of SEQ ID NO:646 shown in FIG. 646.

FIG. 648 shows a nucleotide sequence (SEQ ID NO:648) of a native sequence PRO2023 cDNA, wherein SEQ ID NO:648 is a clone designated herein as “DNA304473”.

FIG. 649 shows the amino acid sequence (SEQ ID NO:649) derived from the coding sequence of SEQ ID NO:648 shown in Figure.

FIG. 650 shows a nucleotide sequence (SEQ ID NO:650) of a native sequence PRO84383 cDNA, wherein SEQ ID NO:650 is a clone designated herein as “DNA328600”.

FIG. 651 shows the amino acid sequence (SEQ ID NO:651) derived from the coding sequence of SEQ ID NO:650 shown in FIG. 650.

FIG. 652 shows a nucleotide sequence (SEQ ID NO:652) of a native sequence PRO84384 cDNA, wherein SEQ ID NO:652 is a clone designated herein as “DNA328601”.

FIG. 653 shows the amino acid sequence (SEQ ID NO:653) derived from the coding sequence of SEQ ID NO:652 shown in FIG. 652.

FIG. 654 shows a nucleotide sequence (SEQ ID NO:654) of a native sequence PRO36800 cDNA, wherein SEQ ID NO:654 is a clone designated herein as “DNA226337”.

FIG. 655 shows the amino acid sequence (SEQ ID NO:655) derived from the coding sequence of SEQ ID NO:654 shown in FIG. 654.

FIG. 656 shows a nucleotide sequence (SEQ ID NO:656) of a native sequence PRO37797 cDNA, wherein SEQ ID NO:656 is a clone designated herein as “DNA227334”.

FIG. 657 shows the amino acid sequence (SEQ ID NO:657) derived from the coding sequence of SEQ ID NO:656 shown in FIG. 656.

FIG. 658 shows a nucleotide sequence (SEQ ID NO:658) of a native sequence PRO24924 cDNA, wherein SEQ ID NO:658 is a clone designated herein as “DNA196426”.

FIG. 659 shows the amino acid sequence (SEQ ID NO:659) derived from the coding sequence of SEQ ID NO:658 shown in FIG. 658.

FIG. 660 shows a nucleotide sequence (SEQ ID NO:660) of a native sequence PRO84802 cDNA, wherein SEQ ID NO:660 is a clone designated herein as “DNA329179”.

FIG. 661 shows the amino acid sequence (SEQ ID NO:661) derived from the coding sequence of SEQ ID NO:660 shown in FIG. 660.

FIG. 662 shows a nucleotide sequence (SEQ ID NO:662) of a native sequence PRO37393 cDNA, wherein SEQ ID NO:662 is a clone designated herein as “DNA226930”.

FIG. 663 shows the amino acid sequence (SEQ ID NO:663) derived from the coding sequence of SEQ ID NO:662 shown in FIG. 662.

FIG. 664 shows a nucleotide sequence (SEQ ID NO:664) of a native sequence PRO84803 cDNA, wherein SEQ ID NO:664 is a clone designated herein as “DNA329180”.

FIG. 665 shows the amino acid sequence (SEQ ID NO:665) derived from the coding sequence of SEQ ID NO:664 shown in FIG. 664.

FIG. 666 shows a nucleotide sequence (SEQ ID NO:666) of a native sequence PRO84804 cDNA, wherein SEQ ID NO:666 is a clone designated herein as “DNA329181”.

FIG. 667 shows the amino acid sequence (SEQ ID NO:667) derived from the coding sequence of SEQ ID NO:666 shown in FIG. 666.

FIG. 668 shows a nucleotide sequence (SEQ ID NO:668) of a native sequence PRO84805 cDNA, wherein SEQ ID NO:668 is a clone designated herein as “DNA329182”.

FIG. 669 shows the amino acid sequence (SEQ ID NO:669) derived from the coding sequence of SEQ ID NO:668 shown in FIG. 668.

FIG. 670 shows a nucleotide sequence (SEQ ID NO:670) of a native sequence PRO21795 cDNA, wherein SEQ ID NO:670 is a clone designated herein as “DNA188271”.

FIG. 671 shows the amino acid sequence (SEQ ID NO:671) derived from the coding sequence of SEQ ID NO:670 shown in FIG. 670.

FIG. 672 shows a nucleotide sequence (SEQ ID NO:672) of a native sequence PRO34286 cDNA, wherein SEQ ID NO:672 is a clone designated herein as “DNA217244”.

FIG. 673 shows the amino acid sequence (SEQ ID NO:673) derived from the coding sequence of SEQ ID NO:672 shown in FIG. 672.

FIG. 674 shows a nucleotide sequence (SEQ ID NO:674) of a native sequence PRO34287 cDNA, wherein SEQ ID NO:674 is a clone designated herein as “DNA217245”.

FIG. 675 shows the amino acid sequence (SEQ ID NO:675) derived from the coding sequence of SEQ ID NO:674 shown in FIG. 674.

FIG. 676 shows a nucleotide sequence (SEQ ID NO:676) of a native sequence PRO34447 cDNA, wherein SEQ ID NO:676 is a clone designated herein as “DNA218651”.

FIG. 677 shows the amino acid sequence (SEQ ID NO:377) derived from the coding sequence of SEQ ID NO:676 shown in FIG. 676.

FIG. 678 shows a nucleotide sequence (SEQ ID NO:678) of a native sequence PRO36753 cDNA, wherein SEQ ID NO:678 is a clone designated herein as “DNA226290”.

FIG. 679 shows the amino acid sequence (SEQ ID NO:679) derived from the coding sequence of SEQ ID NO:678 shown in FIG. 678.

FIG. 680 shows a nucleotide sequence (SEQ ID NO:680) of a native sequence PRO84806 cDNA, wherein SEQ ID NO:680 is a clone designated herein as “DNA329183”.

FIG. 681 shows the amino acid sequence (SEQ ID NO:681) derived from the coding sequence of SEQ ID NO:680 shown in FIG. 680.

FIG. 682 shows a nucleotide sequence (SEQ ID NO:682) of a native sequence PRO84807 cDNA, wherein SEQ ID NO:682 is a clone designated herein as “DNA329184”.

FIG. 683 shows the amino acid sequence (SEQ ID NO:683) derived from the coding sequence of SEQ ID NO:682 shown in FIG. 682.

FIG. 684 shows a nucleotide sequence (SEQ ID NO:684) of a native sequence PRO37687 cDNA, wherein SEQ ID NO:684 is a clone designated herein as “DNA227224”.

FIG. 685 shows the amino acid sequence (SEQ ID NO:685) derived from the coding sequence of SEQ ID NO:684 shown in FIG. 684.

FIG. 686 shows a nucleotide sequence (SEQ ID NO:686) of a native sequence PRO61770 cDNA, wherein SEQ ID NO:686 is a clone designated herein as “DNA329185”.

FIG. 687 shows the amino acid sequence (SEQ ID NO:687) derived from the coding sequence of SEQ ID NO:686 shown in FIG. 686.

FIG. 688 shows a nucleotide sequence (SEQ ID NO:688) of a native sequence PRO80638 cDNA, wherein SEQ ID NO:688 is a clone designated herein as “DNA323896”.

FIG. 689 shows the amino acid sequence (SEQ ID NO:689) derived from the coding sequence of SEQ ID NO:688 shown in FIG. 688.

FIG. 690 shows a nucleotide sequence (SEQ ID NO:690) of a native sequence PRO84391 cDNA, wherein SEQ ID NO:690 is a clone designated herein as “DNA328609”.

FIG. 691 shows the amino acid sequence (SEQ ID NO:691) derived from the coding sequence of SEQ ID NO:690 shown in FIG. 690.

FIG. 692 shows a nucleotide sequence (SEQ ID NO:692) of a native sequence PRO81872 cDNA, wherein SEQ ID NO:692 is a clone designated herein as “DNA325329”.

FIG. 693 shows the amino acid sequence (SEQ ID NO:693) derived from the coding sequence of SEQ ID NO:692 shown in FIG. 692.

FIG. 694 shows a nucleotide sequence (SEQ ID NO:694) of a native sequence PRO72 cDNA, wherein SEQ ID NO:694 is a clone designated herein as “DNA36717”.

FIG. 695 shows the amino acid sequence (SEQ ID NO:695) derived from the coding sequence of SEQ ID NO:694 shown in FIG. 694.

FIG. 696 shows a nucleotide sequence (SEQ ID NO:696) of a native sequence PRO84393 cDNA, wherein SEQ ID NO:696 is a clone designated herein as “DNA328611”.

FIG. 697 shows the amino acid sequence (SEQ ID NO:697) derived from the coding sequence of SEQ ID NO:696 shown in FIG. 696.

FIG. 698 shows a nucleotide sequence (SEQ ID NO:698) of a native sequence PRO82391 cDNA, wherein SEQ ID NO:698 is a clone designated herein as “DNA325944”.

FIG. 699 shows the amino acid sequence (SEQ ID NO:699) derived from the coding sequence of SEQ ID NO:698 shown in FIG. 698.

FIG. 700 shows a nucleotide sequence (SEQ ID NO:700) of a native sequence PRO9824 cDNA, wherein SEQ ID NO:700 is a clone designated herein as “DNA327689”.

FIG. 701 shows the amino acid sequence (SEQ ID NO:701) derived from the coding sequence of SEQ ID NO:700 shown in FIG. 700.

FIG. 702 shows a nucleotide sequence (SEQ ID NO:702) of a native sequence PRO84808 cDNA, wherein SEQ ID NO:702 is a clone designated herein as “DNA329186”.

FIG. 703 shows the amino acid sequence (SEQ ID NO:703) derived from the coding sequence of SEQ ID NO:702 shown in FIG. 702.

FIG. 704 shows a nucleotide sequence (SEQ ID NO:704) of a native sequence PRO84809 cDNA, wherein SEQ ID NO:704 is a clone designated herein as “DNA329187”.

FIG. 705 shows the amino acid sequence (SEQ ID NO:705) derived from the coding sequence of SEQ ID NO:704 shown in FIG. 704.

FIG. 706 shows a nucleotide sequence (SEQ ID NO:706) of a native sequence PRO61545 cDNA, wherein SEQ ID NO:706 is a clone designated herein as “DNA273567”.

FIG. 707 shows the amino acid sequence (SEQ ID NO:707) derived from the coding sequence of SEQ ID NO:706 shown in FIG. 706.

FIG. 708 shows a nucleotide sequence (SEQ ID NO:708) of a native sequence PRO66271 cDNA, wherein SEQ ID NO:708 is a clone designated herein as “DNA281431”.

FIG. 709 shows the amino acid sequence (SEQ ID NO:709) derived from the coding sequence of SEQ ID NO:708 shown in FIG. 708.

FIG. 710 shows a nucleotide sequence (SEQ ID NO:710) of a native sequence PRO84810 cDNA, wherein SEQ ID NO:710 is a clone designated herein as “DNA329188”.

FIG. 711 shows the amino acid sequence (SEQ ID NO:711) derived from the coding sequence of SEQ ID NO:710 shown in FIG. 710.

FIG. 712 shows a nucleotide sequence (SEQ ID NO:712) of a native sequence PRO59579 cDNA, wherein SEQ ID NO:712 is a clone designated herein as “DNA271268”.

FIG. 713 shows the amino acid sequence (SEQ ID NO:713) derived from the coding sequence of SEQ ID NO:712 shown in FIG. 712.

FIG. 714 shows a nucleotide sequence (SEQ ID NO:714) of a native sequence PRO69487 cDNA, wherein SEQ ID NO:714 is a clone designated herein as “DNA287203”.

FIG. 715 shows the amino acid sequence (SEQ ID NO:715) derived from the coding sequence of SEQ ID NO:714 shown in FIG. 714.

FIG. 716 shows a nucleotide sequence (SEQ ID NO:716) of a native sequence PRO71112 cDNA, wherein SEQ ID NO:716 is a clone designated herein as “DNA304686”.

FIG. 717 shows the amino acid sequence (SEQ ID NO:717) derived from the coding sequence of SEQ ID NO:716 shown in FIG. 716.

FIG. 718 shows a nucleotide sequence (SEQ ID NO:718) of a native sequence PRO84401 cDNA, wherein SEQ ID NO:718 is a clone designated herein as “DNA328619”.

FIG. 719 shows the amino acid sequence (SEQ ID NO:719) derived from the coding sequence of SEQ ID NO:718 shown in FIG. 718.

FIG. 720 shows a nucleotide sequence (SEQ ID NO:720) of a native sequence PRO69475 cDNA, wherein SEQ ID NO:720 is a clone designated herein as “DNA287189”.

FIG. 721 shows the amino acid sequence (SEQ ID NO:721) derived from the coding sequence of SEQ ID NO:720 shown in FIG. 720.

FIG. 722 shows a nucleotide sequence (SEQ ID NO:722) of a native sequence PRO84710 cDNA, wherein SEQ ID NO:722 is a clone designated herein as “DNA329048”.

FIG. 723 shows the amino acid sequence (SEQ ID NO:723) derived from the coding sequence of SEQ ID NO:722 shown in FIG. 722.

FIG. 724 shows a nucleotide sequence (SEQ ID NO:724) of a native sequence PRO3645 cDNA, wherein SEQ ID NO:724 is a clone designated herein as “DNA97298”.

FIG. 725 shows the amino acid sequence (SEQ ID NO:725) derived from the coding sequence of SEQ ID NO:724 shown in FIG. 724.

FIG. 726 shows a nucleotide sequence (SEQ ID NO:726) of a native sequence PRO83679 cDNA, wherein SEQ ID NO:726 is a clone designated herein as “DNA327696”.

FIG. 727 shows the amino acid sequence (SEQ ID NO:727) derived from the coding sequence of SEQ ID NO:726 shown in FIG. 726.

FIG. 728 shows a nucleotide sequence (SEQ ID NO:728) of a native sequence PRO69684 cDNA, wherein SEQ ID NO:728 is a clone designated herein as “DNA287427”.

FIG. 729 shows the amino acid sequence (SEQ ID NO:729) derived from the coding sequence of SEQ ID NO:728 shown in FIG. 728.

FIG. 730 shows a nucleotide sequence (SEQ ID NO:730) of a native sequence PRO69498 cDNA, wherein SEQ ID NO:730 is a clone designated herein as “DNA287219”.

FIG. 731 shows the amino acid sequence (SEQ ID NO:731) derived from the coding sequence of SEQ ID NO:730 shown in FIG. 730.

FIG. 732 shows a nucleotide sequence (SEQ ID NO:732) of a native sequence PRO4911 cDNA, wherein SEQ ID NO:732 is a clone designated herein as “DNA329189”.

FIG. 733 shows the amino acid sequence (SEQ ID NO:733) derived from the coding sequence of SEQ ID NO:732 shown in FIG. 732.

FIG. 734 shows a nucleotide sequence (SEQ ID NO:734) of a native sequence PRO82935 cDNA, wherein SEQ ID NO:734 is a clone designated herein as “DNA326573”.

FIG. 735 shows the amino acid sequence (SEQ ID NO:735) derived from the coding sequence of SEQ ID NO:734 shown in FIG. 734.

FIG. 736 shows a nucleotide sequence (SEQ ID NO:736) of a native sequence PRO84403 cDNA, wherein SEQ ID NO:736 is a clone designated herein as “DNA328621”.

FIG. 737 shows the amino acid sequence (SEQ ID NO:737) derived from the coding sequence of SEQ ID NO:736 shown in FIG. 736.

FIG. 738 shows a nucleotide sequence (SEQ ID NO:738) of a native sequence PRO84811 cDNA, wherein SEQ ID NO:738 is a clone designated herein as “DNA329190”.

FIG. 739 shows the amino acid sequence (SEQ ID NO:739) derived from the coding sequence of SEQ ID NO:738 shown in FIG. 738.

FIG. 740 shows a nucleotide sequence (SEQ ID NO:740) of a native sequence PRO10404 cDNA, wherein SEQ ID NO:740 is a clone designated herein as “DNA287169”.

FIG. 741 shows the amino acid sequence (SEQ ID NO:741) derived from the coding sequence of SEQ ID NO:740 shown in FIG. 740.

FIG. 742 shows a nucleotide sequence (SEQ ID NO:742) of a native sequence PRO61502 cDNA, wherein SEQ ID NO:742 is a clone designated herein as “DNA273521”.

FIG. 743 shows the amino acid sequence (SEQ ID NO:743) derived from the coding sequence of SEQ ID NO:742 shown in FIG. 742.

FIG. 744 shows a nucleotide sequence (SEQ ID NO:744) of a native sequence PRO83682 cDNA, wherein SEQ ID NO:744 is a clone designated herein as “DNA327699”.

FIG. 745 shows the amino acid sequence (SEQ ID NO:745) derived from the coding sequence of SEQ ID NO:744 shown in Figure.

FIG. 746 shows a nucleotide sequence (SEQ ID NO:746) of a native sequence PRO80665 cDNA, wherein SEQ ID NO:746 is a clone designated herein as “DNA329191”.

FIG. 747 shows the amino acid sequence (SEQ ID NO:747) derived from the coding sequence of SEQ ID NO:746 shown in FIG. 746.

FIG. 748 shows a nucleotide sequence (SEQ ID NO:748) of a native sequence PRO38019 cDNA, wherein SEQ ID NO:748 is a clone designated herein as “DNA227556”.

FIG. 749 shows the amino acid sequence (SEQ ID NO:749) derived from the coding sequence of SEQ ID NO:748 shown in FIG. 748.

FIG. 750 shows a nucleotide sequence (SEQ ID NO:750) of a native sequence PRO38337 cDNA, wherein SEQ ID NO:750 is a clone designated herein as “DNA227874”.

FIG. 751 shows the amino acid sequence (SEQ ID NO:751) derived from the coding sequence of SEQ ID NO:750 shown in FIG. 750.

FIG. 752 shows a nucleotide sequence (SEQ ID NO:752) of a native sequence PRO83683 cDNA, wherein SEQ ID NO:752 is a clone designated herein as “DNA327700”.

FIG. 753 shows the amino acid sequence (SEQ ID NO:753) derived from the coding sequence of SEQ ID NO:752 shown in FIG. 752.

FIG. 754 shows a nucleotide sequence (SEQ ID NO:754) of a native sequence PRO82667 cDNA, wherein SEQ ID NO:754 is a clone designated herein as “DNA327701”.

FIG. 755 shows the amino acid sequence (SEQ ID NO:755) derived from the coding sequence of SEQ ID NO:754 shown in FIG. 754.

FIG. 756 shows a nucleotide sequence (SEQ ID NO:756) of a native sequence PRO83684 cDNA, wherein SEQ ID NO:756 is a clone designated herein as “DNA327702”.

FIG. 757 shows the amino acid sequence (SEQ ID NO:757) derived from the coding sequence of SEQ ID NO:756 shown in FIG. 576.

FIG. 758 shows a nucleotide sequence (SEQ ID NO:758) of a native sequence PRO70339 cDNA, wherein SEQ ID NO:758 is a clone designated herein as “DNA290237”.

FIG. 759 shows the amino acid sequence (SEQ ID NO:759) derived from the coding sequence of SEQ ID NO:758 shown in FIG. 758.

FIG. 760 shows a nucleotide sequence (SEQ ID NO:760) of a native sequence PRO82739 cDNA, wherein SEQ ID NO:760 is a clone designated herein as “DNA326343”.

FIG. 761 shows the amino acid sequence (SEQ ID NO:761) derived from the coding sequence of SEQ ID NO:760 shown in FIG. 760.

FIG. 762 shows a nucleotide sequence (SEQ ID NO:762) of a native sequence PRO84407 cDNA, wherein SEQ ID NO:762 is a clone designated herein as “DNA328629”.

FIG. 763 shows the amino acid sequence (SEQ ID NO:763) derived from the coding sequence of SEQ ID NO:762 shown in FIG. 762.

FIG. 764 shows a nucleotide sequence (SEQ ID NO:764) of a native sequence PRO84812 cDNA, wherein SEQ ID NO:764 is a clone designated herein as “DNA329192”.

FIG. 765 shows the amino acid sequence (SEQ ID NO:765) derived from the coding sequence of SEQ ID NO:764 shown in FIG. 764.

FIG. 766 shows a nucleotide sequence (SEQ ID NO:766) of a native sequence PRO84411 cDNA, wherein SEQ ID NO:766 is a clone designated herein as “DNA328633”.

FIG. 767 shows the amino acid sequence (SEQ ID NO:767) derived from the coding sequence of SEQ ID NO:766 shown in FIG. 766.

FIG. 768 shows a nucleotide sequence (SEQ ID NO:768) of a native sequence PRO83795 cDNA, wherein SEQ ID NO:768 is a clone designated herein as “DNA327851”.

FIG. 769 shows the amino acid sequence (SEQ ID NO:769) derived from the coding sequence of SEQ ID NO:768 shown in FIG. 768.

FIG. 770 shows a nucleotide sequence (SEQ ID NO:770) of a native sequence PRO83688 cDNA, wherein SEQ ID NO:770 is a clone designated herein as “DNA327706”.

FIG. 771 shows the amino acid sequence (SEQ ID NO:771) derived from the coding sequence of SEQ ID NO:770 shown in FIG. 770.

FIG. 772 shows a nucleotide sequence (SEQ ID NO:772) of a native sequence PRO84413 cDNA, wherein SEQ ID NO:772 is a clone designated herein as “DNA328635”.

FIG. 773 shows the amino acid sequence (SEQ ID NO:773) derived from the coding sequence of SEQ ID NO:772 shown in FIG. 772.

FIG. 774 shows a nucleotide sequence (SEQ ID NO:774) of a native sequence PRO62131 cDNA, wherein SEQ ID NO:774 is a clone designated herein as “DNA274202”.

FIG. 775 shows the amino acid sequence (SEQ ID NO:775) derived from the coding sequence of SEQ ID NO:774 shown in FIG. 774.

FIG. 776 shows a nucleotide sequence (SEQ ID NO:776) of a native sequence PRO84813 cDNA, wherein SEQ ID NO:776 is a clone designated herein as “DNA329193”.

FIG. 777 shows the amino acid sequence (SEQ ID NO:777) derived from the coding sequence of SEQ ID NO:776 shown in FIG. 776.

FIG. 778 shows a nucleotide sequence (SEQ ID NO:778) of a native sequence PRO84814 cDNA, wherein SEQ ID NO:778 is a clone designated herein as “DNA329194”.

FIG. 779 shows the amino acid sequence (SEQ ID NO:779) derived from the coding sequence of SEQ ID NO:778 shown in FIG. 778.

FIG. 780 shows a nucleotide sequence (SEQ ID NO:780) of a native sequence PRO82573 cDNA, wherein SEQ ID NO:780 is a clone designated herein as “DNA329195”.

FIG. 781 shows the amino acid sequence (SEQ ID NO:781) derived from the coding sequence of SEQ ID NO:780 shown in FIG. 780.

FIG. 782 shows a nucleotide sequence (SEQ ID NO:782) of a native sequence PRO81832 cDNA, wherein SEQ ID NO:782 is a clone designated herein as “DNA325285”.

FIG. 783 shows the amino acid sequence (SEQ ID NO:783) derived from the coding sequence of SEQ ID NO:782 shown in FIG. 782.

FIG. 784 shows a nucleotide sequence (SEQ ID NO:784) of a native sequence PRO84815 cDNA, wherein SEQ ID NO:784 is a clone designated herein as “DNA329196”.

FIG. 785 shows the amino acid sequence (SEQ ID NO:785) derived from the coding sequence of SEQ ID NO:784 shown in FIG. 784.

FIG. 786 shows a nucleotide sequence (SEQ ID NO:786) of a native sequence PRO84418 cDNA, wherein SEQ ID NO:786 is a clone designated herein as “DNA328642”.

FIG. 787 shows the amino acid sequence (SEQ ID NO:787) derived from the coding sequence of SEQ ID NO:786 shown in FIG. 786.

FIG. 788 shows a nucleotide sequence (SEQ ID NO:788) of a native sequence PRO84419 cDNA, wherein SEQ ID NO:788 is a clone designated herein as “DNA328643”.

FIG. 789 shows the amino acid sequence (SEQ ID NO:789) derived from the coding sequence of SEQ ID NO:788 shown in FIG. 788.

FIG. 790 shows a nucleotide sequence (SEQ ID NO:790) of a native sequence PRO81387 cDNA, wherein SEQ ID NO:790 is a clone designated herein as “DNA324766”.

FIG. 791 shows the amino acid sequence (SEQ ID NO:791) derived from the coding sequence of SEQ ID NO:790 shown in FIG. 790.

FIG. 792 shows a nucleotide sequence (SEQ ID NO:792) of a native sequence PRO82674 cDNA, wherein SEQ ID NO:792 is a clone designated herein as “DNA326267”.

FIG. 793 shows the amino acid sequence (SEQ ID NO:793) derived from the coding sequence of SEQ ID NO:792 shown in FIG. 792.

FIG. 794 shows a nucleotide sequence (SEQ ID NO:794) of a native sequence PRO59443 cDNA, wherein SEQ ID NO:794 is a clone designated herein as “DNA329197”.

FIG. 795 shows the amino acid sequence (SEQ ID NO:795) derived from the coding sequence of SEQ ID NO:794 shown in FIG. 794.

FIG. 796 shows a nucleotide sequence (SEQ ID NO:796) of a native sequence PRO59258 cDNA, wherein SEQ ID NO:796 is a clone designated herein as “DNA329198”.

FIG. 797 shows the amino acid sequence (SEQ ID NO:797) derived from the coding sequence of SEQ ID NO:796 shown in FIG. 796.

FIG. 798 shows a nucleotide sequence (SEQ ID NO:798) of a native sequence PRO84816 cDNA, wherein SEQ ID NO:798 is a clone designated herein as “DNA329199”.

FIG. 799 shows the amino acid sequence (SEQ ID NO:799) derived from the coding sequence of SEQ ID NO:798 shown in FIG. 798.

FIG. 800 shows a nucleotide sequence (SEQ ID NO:800) of a native sequence PRO84424 cDNA, wherein SEQ ID NO:800 is a clone designated herein as “DNA328649”.

FIG. 801 shows the amino acid sequence (SEQ ID NO:801) derived from the coding sequence of SEQ ID NO:800 shown in FIG. 800.

FIG. 802 shows a nucleotide sequence (SEQ ID NO:802) of a native sequence PRO58159 cDNA, wherein SEQ ID NO:802 is a clone designated herein as “DNA269750”.

FIG. 803 shows the amino acid sequence (SEQ ID NO:803) derived from the coding sequence of SEQ ID NO:802 shown in FIG. 802.

FIG. 804 shows a nucleotide sequence (SEQ ID NO:804) of a native sequence PRO84817 cDNA, wherein SEQ ID NO:804 is a clone designated herein as “DNA329200”.

FIG. 805 shows the amino acid sequence (SEQ ID NO:805) derived from the coding sequence of SEQ ID NO:804 shown in FIG. 804.

FIG. 806 shows a nucleotide sequence (SEQ ID NO:806) of a native sequence PRO62821 cDNA, wherein SEQ ID NO:806 is a clone designated herein as “DNA275106”.

FIG. 807 shows the amino acid sequence (SEQ ID NO:807) derived from the coding sequence of SEQ ID NO:806 shown in FIG. 806.

FIG. 808 shows a nucleotide sequence (SEQ ID NO:808) of a native sequence PRO58042 cDNA, wherein SEQ ID NO:808 is a clone designated herein as “DNA269630”.

FIG. 809 shows the amino acid sequence (SEQ ID NO:809) derived from the coding sequence of SEQ ID NO:808 shown in FIG. 808.

FIG. 810A-B shows a nucleotide sequence (SEQ ID NO:810) of a native sequence PRO84432 cDNA, wherein SEQ ID NO:810 is a clone designated herein as “DNA328658”.

FIG. 811 shows the amino acid sequence (SEQ ID NO:811) derived from the coding sequence of SEQ ID NO:810 shown in FIG. 810A-B.

FIG. 812 shows a nucleotide sequence (SEQ ID NO:812) of a native sequence PRO49214 cDNA, wherein SEQ ID NO:812 is a clone designated herein as “DNA253811”.

FIG. 813 shows the amino acid sequence (SEQ ID NO:813) derived from the coding sequence of SEQ ID NO:812 shown in FIG. 812.

FIG. 814 shows a nucleotide sequence (SEQ ID NO:814) of a native sequence PRO36183 cDNA, wherein SEQ ID NO:814 is a clone designated herein as “DNA328663”.

FIG. 815 shows the amino acid sequence (SEQ ID NO:815) derived from the coding sequence of SEQ ID NO:814 shown in FIG. 814.

FIG. 816 shows a nucleotide sequence (SEQ ID NO:816) of a native sequence PRO84818 cDNA, wherein SEQ ID NO:816 is a clone designated herein as “DNA329201”.

FIG. 817 shows the amino acid sequence (SEQ ID NO:817) derived from the coding sequence of SEQ ID NO:816 shown in FIG. 816.

FIG. 818 shows a nucleotide sequence (SEQ ID NO:818) of a native sequence PRO70367 cDNA, wherein SEQ ID NO:818 is a clone designated herein as “DNA329202”.

FIG. 819 shows the amino acid sequence (SEQ ID NO:819) derived from the coding sequence of SEQ ID NO:818 shown in FIG. 818.

FIG. 820 shows a nucleotide sequence (SEQ ID NO:820) of a native sequence PRO84819 cDNA, wherein SEQ ID NO:820 is a clone designated herein as “DNA329203”.

FIG. 821 shows the amino acid sequence (SEQ ID NO:821) derived from the coding sequence of SEQ ID NO:820 shown in FIG. 820.

FIG. 822 shows a nucleotide sequence (SEQ ID NO:822) of a native sequence PRO60104 cDNA, wherein SEQ ID NO:822 is a clone designated herein as “DNA271823”.

FIG. 823 shows the amino acid sequence (SEQ ID NO:823) derived from the coding sequence of SEQ ID NO:822 shown in FIG. 822.

FIG. 824 shows a nucleotide sequence (SEQ ID NO:824) of a native sequence PRO84382 cDNA, wherein SEQ ID NO:824 is a clone designated herein as “DNA328599”.

FIG. 825 shows the amino acid sequence (SEQ ID NO:825) derived from the coding sequence of SEQ ID NO:824 shown in FIG. 824.

FIG. 826 shows a nucleotide sequence (SEQ ID NO:826) of a native sequence PRO cDNA, wherein SEQ ID NO:826 is a clone designated herein as “DNA”.

FIG. 827 shows the amino acid sequence (SEQ ID NO:827) derived from the coding sequence of SEQ ID NO:826 shown in FIG. 826.

FIG. 828 shows a nucleotide sequence (SEQ ID NO:828) of a native sequence PRO cDNA, wherein SEQ ID NO:828 is a clone designated herein as “DNA”.

FIG. 829 shows the amino acid sequence (SEQ ID NO:829) derived from the coding sequence of SEQ ID NO:828 shown in FIG. 828.

FIG. 830 shows a nucleotide sequence (SEQ ID NO:830) of a native sequence PRO84820 cDNA, wherein SEQ ID NO:830 is a clone designated herein as “DNA329204”.

FIG. 831 shows the amino acid sequence (SEQ ID NO:831) derived from the coding sequence of SEQ ID NO:830 shown in FIG. 830.

FIG. 832 shows a nucleotide sequence (SEQ ID NO:832) of a native sequence PRO34255 cDNA, wherein SEQ ID NO:832 is a clone designated herein as “DNA216503”.

FIG. 833 shows the amino acid sequence (SEQ ID NO:833) derived from the coding sequence of SEQ ID NO:832 shown in FIG. 832.

FIG. 834 shows a nucleotide sequence (SEQ ID NO:834) of a native sequence PRO84821 cDNA, wherein SEQ ID NO:834 is a clone designated herein as “DNA329205”.

FIG. 835 shows the amino acid sequence (SEQ ID NO:835) derived from the coding sequence of SEQ ID NO:834 shown in FIG. 834.

FIG. 836 shows a nucleotide sequence (SEQ ID NO:836) of a native sequence PRO36899 cDNA, wherein SEQ ID NO:836 is a clone designated herein as “DNA226436”.

FIG. 837 shows the amino acid sequence (SEQ ID NO:837) derived from the coding sequence of SEQ ID NO:836 shown in FIG. 836.

FIG. 838 shows a nucleotide sequence (SEQ ID NO:838) of a native sequence PRO84822 cDNA, wherein SEQ ID NO:838 is a clone designated herein as “DNA329206”.

FIG. 839 shows the amino acid sequence (SEQ ID NO:839) derived from the coding sequence of SEQ ID NO:838 shown in FIG. 838.

FIG. 840 shows a nucleotide sequence (SEQ ID NO:840) of a native sequence PRO61801 cDNA, wherein SEQ ID NO:840 is a clone designated herein as “DNA327732”.

FIG. 841 shows the amino acid sequence (SEQ ID NO:841) derived from the coding sequence of SEQ ID NO:840 shown in FIG. 840.

FIG. 842 shows a nucleotide sequence (SEQ ID NO:842) of a native sequence PRO84448 cDNA, wherein SEQ ID NO:842 is a clone designated herein as “DNA328676”.

FIG. 843 shows the amino acid sequence (SEQ ID NO:843) derived from the coding sequence of SEQ ID NO:842 shown in FIG. 842.

FIG. 844 shows a nucleotide sequence (SEQ ID NO:844) of a native sequence PRO84448 cDNA, wherein SEQ ID NO:844 is a clone designated herein as “DNA328676”.

FIG. 845 shows the amino acid sequence (SEQ ID NO:845) derived from the coding sequence of SEQ ID NO:844 shown in FIG. 844.

FIG. 846 shows a nucleotide sequence (SEQ ID NO:846) of a native sequence PRO84449 cDNA, wherein SEQ ID NO:846 is a clone designated herein as “DNA328677”.

FIG. 847 shows the amino acid sequence (SEQ ID NO:847) derived from the coding sequence of SEQ ID NO:846 shown in FIG. 846.

FIG. 848 shows a nucleotide sequence (SEQ ID NO:848) of a native sequence PRO220 cDNA, wherein SEQ ID NO:848 is a clone designated herein as “DNA329207”.

FIG. 849 shows the amino acid sequence (SEQ ID NO:849) derived from the coding sequence of SEQ ID NO:848 shown in FIG. 848.

FIG. 850 shows a nucleotide sequence (SEQ ID NO:850) of a native sequence PRO36583 cDNA, wherein SEQ ID NO:850 is a clone designated herein as “DNA226120”.

FIG. 851 shows the amino acid sequence (SEQ ID NO:851) derived from the coding sequence of SEQ ID NO:850 shown in FIG. 850.

FIG. 852 shows a nucleotide sequence (SEQ ID NO:852) of a native sequence PRO84823 cDNA, wherein SEQ ID NO:852 is a clone designated herein as “DNA329208”.

FIG. 853 shows the amino acid sequence (SEQ ID NO:853) derived from the coding sequence of SEQ ID NO:852 shown in FIG. 852.

FIG. 854 shows a nucleotide sequence (SEQ ID NO:854) of a native sequence PRO63226 cDNA, wherein SEQ ID NO:854 is a clone designated herein as “DNA326562”.

FIG. 855 shows the amino acid sequence (SEQ ID NO:855) derived from the coding sequence of SEQ ID NO:854 shown in FIG. 854.

FIG. 856 shows a nucleotide sequence (SEQ ID NO:856) of a native sequence cDNA, wherein SEQ ID NO:856 is a clone designated herein as “DNA154921”.

FIG. 857A-B shows a nucleotide sequence (SEQ ID NO:857) of a native sequence PRO37597 cDNA, wherein SEQ ID NO:857 is a clone designated herein as “DNA227134”.

FIG. 858 shows the amino acid sequence (SEQ ID NO:858) derived from the coding sequence of SEQ ID NO:857 shown in FIG. 857A-B.

FIG. 859A-B shows a nucleotide sequence (SEQ ID NO:859) of a native sequence PRO84824 cDNA, wherein SEQ ID NO:859 is a clone designated herein as “DNA329209”.

FIG. 860 shows the amino acid sequence (SEQ ID NO:860) derived from the coding sequence of SEQ ID NO:859 shown in FIG. 859A-B.

FIG. 861 shows a nucleotide sequence (SEQ ID NO:861) of a native sequence PRO37121 cDNA, wherein SEQ ID NO:861 is a clone designated herein as “DNA226658”.

FIG. 862 shows the amino acid sequence (SEQ ID NO:862) derived from the coding sequence of SEQ ID NO:861 shown in FIG. 861.

FIG. 863 shows a nucleotide sequence (SEQ ID NO:863) of a native sequence PRO82342 cDNA, wherein SEQ ID NO:863 is a clone designated herein as “DNA325886”.

FIG. 864 shows the amino acid sequence (SEQ ID NO:864) derived from the coding sequence of SEQ ID NO:863 shown in FIG. 863.

FIG. 865 shows a nucleotide sequence (SEQ ID NO:865) of a native sequence PRO22818 cDNA, wherein SEQ ID NO:865 is a clone designated herein as “DNA329210”.

FIG. 866 shows the amino acid sequence (SEQ ID NO:866) derived from the coding sequence of SEQ ID NO:865 shown in FIG. 865.

FIG. 867 shows a nucleotide sequence (SEQ ID NO:867) of a native sequence PRO64 cDNA, wherein SEQ ID NO:867 is a clone designated herein as “DNA328686”.

FIG. 868 shows the amino acid sequence (SEQ ID NO:868) derived from the coding sequence of SEQ ID NO:867 shown in FIG. 867.

FIG. 869 shows a nucleotide sequence (SEQ ID NO:869) of a native sequence PRO2081 cDNA, wherein SEQ ID NO:869 is a clone designated herein as “DNA287620”.

FIG. 870 shows the amino acid sequence (SEQ ID NO:870) derived from the coding sequence of SEQ ID NO:869 shown in FIG. 869.

FIG. 871 shows a nucleotide sequence (SEQ ID NO:871) of a native sequence PRO7078 cDNA, wherein SEQ ID NO:871 is a clone designated herein as “DNA329211”.

FIG. 872 shows the amino acid sequence (SEQ ID NO:872) derived from the coding sequence of SEQ ID NO:871 shown in FIG. 871.

FIG. 873 shows a nucleotide sequence (SEQ ID NO:873) of a native sequence PRO80521 cDNA, wherein SEQ ID NO:873 is a clone designated herein as “DNA323769”.

FIG. 874 shows the amino acid sequence (SEQ ID NO:874) derived from the coding sequence of SEQ ID NO:873 shown in FIG. 873.

FIG. 875 shows a nucleotide sequence (SEQ ID NO:875) of a native sequence PRO84825 cDNA, wherein SEQ ID NO:875 is a clone designated herein as “DNA329212”.

FIG. 876 shows the amino acid sequence (SEQ ID NO:876) derived from the coding sequence of SEQ ID NO:875 shown in FIG. 875.

FIG. 877 shows a nucleotide sequence (SEQ ID NO:877) of a native sequence PRO34295 cDNA, wherein SEQ ID NO:877 is a clone designated herein as “DNA217253”.

FIG. 878 shows the amino acid sequence (SEQ ID NO:878) derived from the coding sequence of SEQ ID NO:877 shown in FIG. 877.

FIG. 879 shows a nucleotide sequence (SEQ ID NO:879) of a native sequence PRO2313 cDNA, wherein SEQ ID NO:879 is a clone designated herein as “DNA329213”.

FIG. 880 shows the amino acid sequence (SEQ ID NO:880) derived from the coding sequence of SEQ ID NO:879 shown in FIG. 879.

FIG. 881A-B shows a nucleotide sequence (SEQ ID NO:881) of a native sequence PRO84826 cDNA, wherein SEQ ID NO:881 is a clone designated herein as “DNA329214”.

FIG. 882 shows the amino acid sequence (SEQ ID NO:882) derived from the coding sequence of SEQ ID NO:881 shown in FIG. 881A-B.

FIG. 883 shows a nucleotide sequence (SEQ ID NO:883) of a native sequence PRO71063 cDNA, wherein SEQ ID NO:883 is a clone designated herein as “DNA304499”.

FIG. 884 shows the amino acid sequence (SEQ ID NO:884) derived from the coding sequence of SEQ ID NO:883 shown in FIG. 883.

FIG. 885 shows a nucleotide sequence (SEQ ID NO:885) of a native sequence PRO35991 cDNA, wherein SEQ ID NO:885 is a clone designated herein as “DNA225528”.

FIG. 886 shows the amino acid sequence (SEQ ID NO:886) derived from the coding sequence of SEQ ID NO:885 shown in FIG. 885.

FIG. 887 shows a nucleotide sequence (SEQ ID NO:887) of a native sequence PRO7424 cDNA, wherein SEQ ID NO:887 is a clone designated herein as “DNA329215”.

FIG. 888 shows the amino acid sequence (SEQ ID NO:888) derived from the coding sequence of SEQ ID NO:887 shown in FIG. 887.

FIG. 889 shows a nucleotide sequence (SEQ ID NO:889) of a native sequence PRO36857 cDNA, wherein SEQ ID NO:889 is a clone designated herein as “DNA226394”.

FIG. 890 shows the amino acid sequence (SEQ ID NO:890) derived from the coding sequence of SEQ ID NO:889 shown in FIG. 889.

FIG. 891 shows a nucleotide sequence (SEQ ID NO:891) of a native sequence PRO61638 cDNA, wherein SEQ ID NO:891 is a clone designated herein as “DNA323902”.

FIG. 892 shows the amino acid sequence (SEQ ID NO:892) derived from the coding sequence of SEQ ID NO:891 shown in FIG. 891.

FIG. 893 shows a nucleotide sequence (SEQ ID NO:893) of a native sequence PRO84827 cDNA, wherein SEQ ID NO:893 is a clone designated herein as “DNA329216”.

FIG. 894 shows the amino acid sequence (SEQ ID NO:894) derived from the coding sequence of SEQ ID NO:893 shown in FIG. 893.

FIG. 895 shows a nucleotide sequence (SEQ ID NO:895) of a native sequence PRO84828 cDNA, wherein SEQ ID NO:895 is a clone designated herein as “DNA329217”.

FIG. 896 shows the amino acid sequence (SEQ ID NO:896) derived from the coding sequence of SEQ ID NO:895 shown in FIG. 895.

FIG. 897 shows a nucleotide sequence (SEQ ID NO:897) of a native sequence PRO84829 cDNA, wherein SEQ ID NO:897 is a clone designated herein as “DNA329218”.

FIG. 898 shows the amino acid sequence (SEQ ID NO:898) derived from the coding sequence of SEQ ID NO:897 shown in FIG. 897.

FIG. 899 shows a nucleotide sequence (SEQ ID NO:899) of a native sequence PRO83721 cDNA, wherein SEQ ID NO:899 is a clone designated herein as “DNA327747”.

FIG. 900 shows the amino acid sequence (SEQ ID NO:900) derived from the coding sequence of SEQ ID NO:899 shown in FIG. 899.

FIG. 901 shows a nucleotide sequence (SEQ ID NO:901) of a native sequence PRO38923 cDNA, wherein SEQ ID NO:901 is a clone designated herein as “DNA237817”.

FIG. 902 shows the amino acid sequence (SEQ ID NO:902) derived from the coding sequence of SEQ ID NO:901 shown in FIG. 901.

FIG. 903 shows a nucleotide sequence (SEQ ID NO:903) of a native sequence PRO81278 cDNA, wherein SEQ ID NO:903 is a clone designated herein as “DNA329219”.

FIG. 904 shows the amino acid sequence (SEQ ID NO:904) derived from the coding sequence of SEQ ID NO:903 shown in FIG. 903.

FIG. 905A-C shows a nucleotide sequence (SEQ ID NO:905) of a native sequence PRO84830 cDNA, wherein SEQ ID NO:905 is a clone designated herein as “DNA329220”.

FIG. 906 shows the amino acid sequence (SEQ ID NO:906) derived from the coding sequence of SEQ ID NO:905 shown in FIG. 905A-C.

FIG. 907 shows a nucleotide sequence (SEQ ID NO:907) of a native sequence PRO50026 cDNA, wherein SEQ ID NO:907 is a clone designated herein as “DNA254936”.

FIG. 908 shows the amino acid sequence (SEQ ID NO:908) derived from the coding sequence of SEQ ID NO:907 shown in FIG. 907.

FIG. 909 shows a nucleotide sequence (SEQ ID NO:909) of a native sequence PRO61971 cDNA, wherein SEQ ID NO:909 is a clone designated herein as “DNA274027”.

FIG. 910 shows the amino acid sequence (SEQ ID NO:910) derived from the coding sequence of SEQ ID NO:909 shown in FIG. 909.

FIG. 911 shows a nucleotide sequence (SEQ ID NO:911) of a native sequence PRO4555 cDNA, wherein SEQ ID NO:911 is a clone designated herein as “DNA329221”.

FIG. 912 shows the amino acid sequence (SEQ ID NO:912) derived from the coding sequence of SEQ ID NO:911 shown in FIG. 911.

FIG. 913 shows a nucleotide sequence (SEQ ID NO:913) of a native sequence PRO61480 cDNA, wherein SEQ ID NO:913 is a clone designated herein as “DNA329222”.

FIG. 914 shows the amino acid sequence (SEQ ID NO:914) derived from the coding sequence of SEQ ID NO:913 shown in FIG. 913.

FIG. 915 shows a nucleotide sequence (SEQ ID NO:915) of a native sequence PRO71178 cDNA, wherein SEQ ID NO:915 is a clone designated herein as “DNA304765”.

FIG. 916 shows the amino acid sequence (SEQ ID NO:916) derived from the coding sequence of SEQ ID NO:915 shown in FIG. 915.

FIG. 917 shows a nucleotide sequence (SEQ ID NO:917) of a native sequence PRO4723 cDNA, wherein SEQ ID NO:917 is a clone designated herein as “DNA103395”.

FIG. 918 shows the amino acid sequence (SEQ ID NO:918) derived from the coding sequence of SEQ ID NO:917 shown in FIG. 917.

FIG. 919A-B shows a nucleotide sequence (SEQ ID NO:919) of a native sequence PRO62786 cDNA, wherein SEQ ID NO:919 is a clone designated herein as “DNA275066”.

FIG. 920 shows the amino acid sequence (SEQ ID NO:920) derived from the coding sequence of SEQ ID NO:919 shown in FIG. 919A-B.

FIG. 921 shows a nucleotide sequence (SEQ ID NO:921) of a native sequence PRO83725 cDNA, wherein SEQ ID NO:921 is a clone designated herein as “DNA327755”.

FIG. 922 shows the amino acid sequence (SEQ ID NO:922) derived from the coding sequence of SEQ ID NO:921 shown in FIG. 921.

FIG. 923 shows a nucleotide sequence (SEQ ID NO:923) of a native sequence PRO84831 cDNA, wherein SEQ ID NO:923 is a clone designated herein as “DNA329223”.

FIG. 924 shows the amino acid sequence (SEQ ID NO:924) derived from the coding sequence of SEQ ID NO:923 shown in FIG. 923.

FIG. 925 shows a nucleotide sequence (SEQ ID NO:925) of a native sequence PRO37041 cDNA, wherein SEQ ID NO:925 is a clone designated herein as “DNA226578”.

FIG. 926 shows the amino acid sequence (SEQ ID NO:926) derived from the coding sequence of SEQ ID NO:925 shown in FIG. 925.

FIG. 927 shows a nucleotide sequence (SEQ ID NO:927) of a native sequence PRO84832 cDNA, wherein SEQ ID NO:927 is a clone designated herein as “DNA329224”.

FIG. 928 shows the amino acid sequence (SEQ ID NO:928) derived from the coding sequence of SEQ ID NO:927 shown in FIG. 927.

FIG. 929 shows a nucleotide sequence (SEQ ID NO:929) of a native sequence PRO10347 cDNA, wherein SEQ ID NO:929 is a clone designated herein as “DNA328706”.

FIG. 930 shows the amino acid sequence (SEQ ID NO:930) derived from the coding sequence of SEQ ID NO:929 shown in FIG. 929.

FIG. 931 shows a nucleotide sequence (SEQ ID NO:931) of a native sequence PRO84833 cDNA, wherein SEQ ID NO:931 is a clone designated herein as “DNA329225”.

FIG. 932 shows the amino acid sequence (SEQ ID NO:932) derived from the coding sequence of SEQ ID NO:931 shown in FIG. 931.

FIG. 933A-B shows a nucleotide sequence (SEQ ID NO:933) of a native sequence PRO60388 cDNA, wherein SEQ ID NO:933 is a clone designated herein as “DNA329226”.

FIG. 934 shows the amino acid sequence (SEQ ID NO:934) derived from the coding sequence of SEQ ID NO:933 shown in FIG. 933A-B.

FIG. 935 shows a nucleotide sequence (SEQ ID NO:935) of a native sequence PRO2023 cDNA, wherein SEQ ID NO:935 is a clone designated herein as “DNA304473”.

FIG. 936 shows the amino acid sequence (SEQ ID NO:936) derived from the coding sequence of SEQ ID NO:935 shown in FIG. 935.

FIG. 937A-B shows a nucleotide sequence (SEQ ID NO:937) of a native sequence PRO34751 cDNA, wherein SEQ ID NO:937 is a clone designated herein as “DNA328685”.

FIG. 938 shows the amino acid sequence (SEQ ID NO:938) derived from the coding sequence of SEQ ID NO:937 shown in FIG. 937A-B.

FIG. 939A-B shows a nucleotide sequence (SEQ ID NO:939) of a native sequence PRO81785 cDNA, wherein SEQ ID NO:939 is a clone designated herein as “DNA325227”.

FIG. 940 shows the amino acid sequence (SEQ ID NO:940) derived from the coding sequence of SEQ ID NO:939 shown in FIG. 939A-B.

FIG. 941A-B shows a nucleotide sequence (SEQ ID NO:941) of a native sequence cDNA, wherein SEQ ID NO:941 is a clone designated herein as “DNA272195”.

FIG. 942A-B shows a nucleotide sequence (SEQ ID NO:942) of a native sequence PRO82307 cDNA, wherein SEQ ID NO:942 is a clone designated herein as “DNA329227”.

FIG. 943 shows the amino acid sequence (SEQ ID NO:943) derived from the coding sequence of SEQ ID NO:942 shown in FIG. 942A-B.

FIG. 944 shows a nucleotide sequence (SEQ ID NO:944) of a native sequence PRO82388 cDNA, wherein SEQ ID NO:944 is a clone designated herein as “DNA325941”.

FIG. 945 shows the amino acid sequence (SEQ ID NO:945) derived from the coding sequence of SEQ ID NO:944 shown in FIG. 944.

FIG. 946 shows a nucleotide sequence (SEQ ID NO:946) of a native sequence PRO69480 cDNA, wherein SEQ ID NO:946 is a clone designated herein as “DNA287194”.

FIG. 947 shows the amino acid sequence (SEQ ID NO:947) derived from the coding sequence of SEQ ID NO:946 shown in FIG. 946.

FIG. 948A-C shows a nucleotide sequence (SEQ ID NO:948) of a native sequence PRO84834 cDNA, wherein SEQ ID NO:948 is a clone designated herein as “DNA329228”.

FIG. 949 shows the amino acid sequence (SEQ ID NO:949) derived from the coding sequence of SEQ ID NO:948 shown in FIG. 948A-C.

FIG. 950 shows a nucleotide sequence (SEQ ID NO:950) of a native sequence PRO69690 cDNA, wherein SEQ ID NO:950 is a clone designated herein as “DNA287433”.

FIG. 951 shows the amino acid sequence (SEQ ID NO:951) derived from the coding sequence of SEQ ID NO:950 shown in FIG. 950.

FIG. 952 shows a nucleotide sequence (SEQ ID NO:952) of a native sequence PRO4710 cDNA, wherein SEQ ID NO:952 is a clone designated herein as “DNA103380”.

FIG. 953 shows the amino acid sequence (SEQ ID NO:953) derived from the coding sequence of SEQ ID NO:952 shown in FIG. 952.

FIG. 954A-B shows a nucleotide sequence (SEQ ID NO:954) of a native sequence PRO12560 cDNA, wherein SEQ ID NO:954 is a clone designated herein as “DNA150956”.

FIG. 955 shows the amino acid sequence (SEQ ID NO:955) derived from the coding sequence of SEQ ID NO:954 shown in FIG. 954A-B.

FIG. 956A-B shows a nucleotide sequence (SEQ ID NO:956) of a native sequence cDNA, wherein SEQ ID NO:956 is a clone designated herein as “DNA150829”.

FIG. 957A-B shows a nucleotide sequence (SEQ ID NO:957) of a native sequence PRO84835 cDNA, wherein SEQ ID NO:957 is a clone designated herein as “DNA329229”.

FIG. 958 shows the amino acid sequence (SEQ ID NO:958) derived from the coding sequence of SEQ ID NO:957 shown in FIG. 957A-B.

FIG. 959 shows a nucleotide sequence (SEQ ID NO:959) of a native sequence PRO84836 cDNA, wherein SEQ ID NO:959 is a clone designated herein as “DNA329230”.

FIG. 960 shows the amino acid sequence (SEQ ID NO:960) derived from the coding sequence of SEQ ID NO:959 shown in FIG. 959.

FIG. 961 shows a nucleotide sequence (SEQ ID NO:961) of a native sequence cDNA, wherein SEQ ID NO:961 is a clone designated herein as “DNA150980”.

FIG. 962 shows a nucleotide sequence (SEQ ID NO:962) of a native sequence PRO84475 cDNA, wherein SEQ ID NO:962 is a clone designated herein as “DNA328719”.

FIG. 963 shows the amino acid sequence (SEQ ID NO:963) derived from the coding sequence of SEQ ID NO:962 shown in FIG. 962.

FIG. 964 shows a nucleotide sequence (SEQ ID NO:964) of a native sequence PRO59425 cDNA, wherein SEQ ID NO:964 is a clone designated herein as “DNA271103”.

FIG. 965 shows the amino acid sequence (SEQ ID NO:965) derived from the coding sequence of SEQ ID NO:964 shown in FIG. 964.

FIG. 966 shows a nucleotide sequence (SEQ ID NO:966) of a native sequence cDNA, wherein SEQ ID NO:966 is a clone designated herein as “DNA207620”.

FIG. 967 shows a nucleotide sequence (SEQ ID NO:967) of a native sequence PRO83141 cDNA, wherein SEQ ID NO:967 is a clone designated herein as “DNA326808”.

FIG. 968 shows the amino acid sequence (SEQ ID NO:968) derived from the coding sequence of SEQ ID NO:967 shown in FIG. 967.

FIG. 969A-B shows a nucleotide sequence (SEQ ID NO:969) of a native sequence PRO6323 cDNA, wherein SEQ ID NO:969 is a clone designated herein as “DNA124122”.

FIG. 970 shows the amino acid sequence (SEQ ID NO:970) derived from the coding sequence of SEQ ID NO:969 shown in FIG. 969A-B.

FIG. 971A-B shows a nucleotide sequence (SEQ ID NO:971) of a native sequence PRO6323 cDNA, wherein SEQ ID NO:971 is a clone designated herein as “DNA124122”.

FIG. 972 shows the amino acid sequence (SEQ ID NO:972) derived from the coding sequence of SEQ ID NO:971 shown in FIG. 971A-B.

FIG. 973 shows a nucleotide sequence (SEQ ID NO:973) of a native sequence PRO69476 cDNA, wherein SEQ ID NO:973 is a clone designated herein as “DNA287190”.

FIG. 974 shows the amino acid sequence (SEQ ID NO:974) derived from the coding sequence of SEQ ID NO:973 shown in FIG. 973.

FIG. 975 shows a nucleotide sequence (SEQ ID NO:975) of a native sequence PRO84837 cDNA, wherein SEQ ID NO:975 is a clone designated herein as “DNA329231”.

FIG. 976 shows the amino acid sequence (SEQ ID NO:976) derived from the coding sequence of SEQ ID NO:975 shown in FIG. 975.

FIG. 977A-B shows a nucleotide sequence (SEQ ID NO:977) of a native sequence PRO12554 cDNA, wherein SEQ ID NO:977 is a clone designated herein as “DNA150950”.

FIG. 978 shows the amino acid sequence (SEQ ID NO:978) derived from the coding sequence of SEQ ID NO:977 shown in FIG. 977A-B.

FIG. 979 shows a nucleotide sequence (SEQ ID NO:979) of a native sequence PRO11708 cDNA, wherein SEQ ID NO:979 is a clone designated herein as “DNA151330”.

FIG. 980 shows the amino acid sequence (SEQ ID NO:980) derived from the coding sequence of SEQ ID NO:979 shown in FIG. 979.

FIG. 981 shows a nucleotide sequence (SEQ ID NO:981) of a native sequence cDNA, wherein SEQ ID NO:981 is a clone designated herein as “DNA329232”.

FIG. 982 shows a nucleotide sequence (SEQ ID NO:982) of a native sequence PRO71082 cDNA, wherein SEQ ID NO:982 is a clone designated herein as “DNA304655”.

FIG. 983 shows the amino acid sequence (SEQ ID NO:983) derived from the coding sequence of SEQ ID NO:982 shown in FIG. 982.

FIG. 984 shows a nucleotide sequence (SEQ ID NO:984) of a native sequence PRO84485 cDNA, wherein SEQ ID NO:984 is a clone designated herein as “DNA328732”.

FIG. 985 shows the amino acid sequence (SEQ ID NO:985) derived from the coding sequence of SEQ ID NO:984 shown in FIG. 984.

FIG. 986 shows a nucleotide sequence (SEQ ID NO:986) of a native sequence PRO84839 cDNA, wherein SEQ ID NO:986 is a clone designated herein as “DNA329233”.

FIG. 987 shows the amino acid sequence (SEQ ID NO:987) derived from the coding sequence of SEQ ID NO:986 shown in FIG. 986.

FIG. 988 shows a nucleotide sequence (SEQ ID NO:988) of a native sequence cDNA, wherein SEQ ID NO:988 is a clone designated herein as “DNA329234”.

FIG. 989A-D shows a nucleotide sequence (SEQ ID NO:989) of a native sequence PRO84490 cDNA, wherein SEQ ID NO:989 is a clone designated herein as “DNA328737”.

FIG. 990 shows the amino acid sequence (SEQ ID NO:990) derived from the coding sequence of SEQ ID NO:989 shown in FIG. 989A-D.

FIG. 991 shows a nucleotide sequence (SEQ ID NO:991) of a native sequence PRO81715 cDNA, wherein SEQ ID NO:991 is a clone designated herein as “DNA329235”.

FIG. 992 shows the amino acid sequence (SEQ ID NO:992) derived from the coding sequence of SEQ ID NO:991 shown in FIG. 991.

FIG. 993A-C shows a nucleotide sequence (SEQ ID NO:993) of a native sequence PRO84841 cDNA, wherein SEQ ID NO:993 is a clone designated herein as “DNA329236”.

FIG. 994 shows the amino acid sequence (SEQ ID NO:994) derived from the coding sequence of SEQ ID NO:993 shown in FIG. 993A-C.

FIG. 995 shows a nucleotide sequence (SEQ ID NO:995) of a native sequence PRO84841 cDNA, wherein SEQ ID NO:995 is a clone designated herein as “DNA329236”.

FIG. 996 shows the amino acid sequence (SEQ ID NO:996) derived from the coding sequence of SEQ ID NO:995 shown in FIG. 995.

FIG. 997 shows a nucleotide sequence (SEQ ID NO:997) of a native sequence PRO11833 cDNA, wherein SEQ ID NO:997 is a clone designated herein as “DNA151487”.

FIG. 998 shows the amino acid sequence (SEQ ID NO:998) derived from the coding sequence of SEQ ID NO:997 shown in FIG. 997.

FIG. 999A-B shows a nucleotide sequence (SEQ ID NO:999) of a native sequence PRO84842 cDNA, wherein SEQ ID NO:999 is a clone designated herein as “DNA329237”.

FIG. 1000 shows the amino acid sequence (SEQ ID NO:1000) derived from the coding sequence of SEQ ID NO:999 shown in FIG. 999A-B.

FIG. 1001 shows a nucleotide sequence (SEQ ID NO:1001) of a native sequence PRO84843 cDNA, wherein SEQ ID NO:1001 is a clone designated herein as “DNA329238”.

FIG. 1002 shows the amino acid sequence (SEQ ID NO:1002) derived from the coding sequence of SEQ ID NO:1001 shown in FIG. 1001.

FIG. 1003A-B shows a nucleotide sequence (SEQ ID NO:1003) of a native sequence cDNA, wherein SEQ ID NO:1003 is a clone designated herein as “DNA327778”.

FIG. 1004A-B shows a nucleotide sequence (SEQ ID NO:1004) of a native sequence cDNA, wherein SEQ ID NO:1004 is a clone designated herein as “DNA287360”.

FIG. 1005A-B shows a nucleotide sequence (SEQ ID NO:1005) of a native sequence cDNA, wherein SEQ ID NO:1005 is a clone designated herein as “DNA270118”.

FIG. 1006A-B shows a nucleotide sequence (SEQ ID NO:1006) of a native sequence PRO59570 cDNA, wherein SEQ ID NO:1006 is a clone designated herein as “DNA328748”.

FIG. 1007 shows the amino acid sequence (SEQ ID NO:1007) derived from the coding sequence of SEQ ID NO:1006 shown in FIG. 1006A-B.

FIG. 1008 shows a nucleotide sequence (SEQ ID NO:1008) of a native sequence PRO84500 cDNA, wherein SEQ ID NO:1008 is a clone designated herein as “DNA328750”.

FIG. 1009 shows the amino acid sequence (SEQ ID NO:1009) derived from the coding sequence of SEQ ID NO:1008 shown in FIG. 1008.

FIG. 1010A-B shows a nucleotide sequence (SEQ ID NO:1010) of a native sequence PRO84502 cDNA, wherein SEQ ID NO:1010 is a clone designated herein as “DNA328753”.

FIG. 1011 shows the amino acid sequence (SEQ ID NO:1011) derived from the coding sequence of SEQ ID NO:1010 shown in FIG. 1010A-B.

FIG. 1012 shows a nucleotide sequence (SEQ ID NO:1012) of a native sequence PRO69549 cDNA, wherein SEQ ID NO:1012 is a clone designated herein as “DNA325596”.

FIG. 1013 shows the amino acid sequence (SEQ ID NO:1013) derived from the coding sequence of SEQ ID NO:1012 shown in FIG. 1012.

FIG. 1014 shows a nucleotide sequence (SEQ ID NO:1014) of a native sequence PRO84844 cDNA, wherein SEQ ID NO:1014 is a clone designated herein as “DNA329239”.

FIG. 1015 shows the amino acid sequence (SEQ ID NO:1015) derived from the coding sequence of SEQ ID NO:1014 shown in FIG. 1014.

FIG. 1016A-B shows a nucleotide sequence (SEQ ID NO:1016) of a native sequence PRO84845 cDNA, wherein SEQ ID NO:1016 is a clone designated herein as “DNA329240”.

FIG. 1017 shows the amino acid sequence (SEQ ID NO:1017) derived from the coding sequence of SEQ ID NO:1016 shown in FIG. 1016.

FIG. 1018 shows a nucleotide sequence (SEQ ID NO:1018) of a native sequence PRO84846 cDNA, wherein SEQ ID NO:1018 is a clone designated herein as “DNA329241”.

FIG. 1019 shows the amino acid sequence (SEQ ID NO:1019) derived from the coding sequence of SEQ ID NO:1018 shown in FIG. 1018.

FIG. 1020A-B shows a nucleotide sequence (SEQ ID NO:1020) of a native sequence PRO84847 cDNA, wherein SEQ ID NO:1020 is a clone designated herein as “DNA329242”.

FIG. 1021 shows the amino acid sequence (SEQ ID NO:1021) derived from the coding sequence of SEQ ID NO:1020 shown in FIG. 1020.

FIG. 1022 shows a nucleotide sequence (SEQ ID NO:1022) of a native sequence PRO84848 cDNA, wherein SEQ ID NO:1022 is a clone designated herein as “DNA329243”.

FIG. 1023 shows the amino acid sequence (SEQ ID NO:1023) derived from the coding sequence of SEQ ID NO:1022 shown in FIG. 1022.

FIG. 1024A-C shows a nucleotide sequence (SEQ ID NO:1024) of a native sequence PRO84849 cDNA, wherein SEQ ID NO:1024 is a clone designated herein as “DNA329244”.

FIG. 1025 shows the amino acid sequence (SEQ ID NO:1025) derived from the coding sequence of SEQ ID NO:1024 shown in FIG. 1024.

FIG. 1026A-B shows a nucleotide sequence (SEQ ID NO:1026) of a native sequence cDNA, wherein SEQ ID NO:1026 is a clone designated herein as “DNA328758”.

FIG. 1027 shows a nucleotide sequence (SEQ ID NO:1027) of a native sequence cDNA, wherein SEQ ID NO:1027 is a clone designated herein as “DNA329245”.

FIG. 1028A-B shows a nucleotide sequence (SEQ ID NO:1028) of a native sequence cDNA, wherein SEQ ID NO:1028 is a clone designated herein as “DNA329246”.

FIG. 1029 shows a nucleotide sequence (SEQ ID NO:1029) of a native sequence PRO69509 cDNA, wherein SEQ ID NO:1029 is a clone designated herein as “DNA287230”.

FIG. 1030 shows the amino acid sequence (SEQ ID NO:1030) derived from the coding sequence of SEQ ID NO:1029 shown in FIG. 1029.

FIG. 1031 shows a nucleotide sequence (SEQ ID NO:1031) of a native sequence cDNA, wherein SEQ ID NO:1031 is a clone designated herein as “DNA228053”.

FIG. 1032 shows a nucleotide sequence (SEQ ID NO:1032) of a native sequence PRO69541 cDNA, wherein SEQ ID NO:1032 is a clone designated herein as “DNA287270”.

FIG. 1033 shows the amino acid sequence (SEQ ID NO:1033) derived from the coding sequence of SEQ ID NO:1032 shown in FIG. 1032.

FIG. 1034A-C shows a nucleotide sequence (SEQ ID NO:1034) of a native sequence PRO59767 cDNA, wherein SEQ ID NO:1034 is a clone designated herein as “DNA329247”.

FIG. 1035 shows the amino acid sequence (SEQ ID NO:1035) derived from the coding sequence of SEQ ID NO:1034 shown in FIG. 1034.

FIG. 1036A-B shows a nucleotide sequence (SEQ ID NO:1036) of a native sequence PRO84850 cDNA, wherein SEQ ID NO:1036 is a clone designated herein as “DNA329248”.

FIG. 1037 shows the amino acid sequence (SEQ ID NO:1037) derived from the coding sequence of SEQ ID NO:1036 shown in FIG. 1036.

FIG. 1038 shows a nucleotide sequence (SEQ ID NO:1038) of a native sequence PRO50349 cDNA, wherein SEQ ID NO:1038 is a clone designated herein as “DNA255273”.

FIG. 1039 shows the amino acid sequence (SEQ ID NO:1039) derived from the coding sequence of SEQ ID NO:1038 shown in FIG. 1038.

FIG. 1040 shows a nucleotide sequence (SEQ ID NO:1040) of a native sequence PRO10641 cDNA, wherein SEQ ID NO:1040 is a clone designated herein as “DNA329249”.

FIG. 1041 shows the amino acid sequence (SEQ ID NO:1041) derived from the coding sequence of SEQ ID NO:1040 shown in FIG. 1040.

FIG. 1042A-B shows a nucleotide sequence (SEQ ID NO:1042) of a native sequence PRO84851 cDNA, wherein SEQ ID NO:1042 is a clone designated herein as “DNA329250”.

FIG. 1043 shows the amino acid sequence (SEQ ID NO:1043) derived from the coding sequence of SEQ ID NO:1042 shown in FIG. 1042.

FIG. 1044A-B shows a nucleotide sequence (SEQ ID NO:1044) of a native sequence cDNA, wherein SEQ ID NO:1044 is a clone designated herein as “DNA329251”.

FIG. 1045 shows a nucleotide sequence (SEQ ID NO:1045) of a native sequence PRO49375 cDNA, wherein SEQ ID NO:1045 is a clone designated herein as “DNA254264”.

FIG. 1046 shows the amino acid sequence (SEQ ID NO:1046) derived from the coding sequence of SEQ ID NO:1045 shown in FIG. 1045.

FIG. 1047 shows a nucleotide sequence (SEQ ID NO:1047) of a native sequence PRO83763 cDNA, wherein SEQ ID NO:1047 is a clone designated herein as “DNA327800”.

FIG. 1048 shows the amino acid sequence (SEQ ID NO:1048) derived from the coding sequence of SEQ ID NO:1047 shown in FIG. 1047.

FIG. 1049 shows a nucleotide sequence (SEQ ID NO:1049) of a native sequence PRO82188 cDNA, wherein SEQ ID NO:1049 is a clone designated herein as “DNA325704”.

FIG. 1050 shows the amino acid sequence (SEQ ID NO:1050) derived from the coding sequence of SEQ ID NO:1049 shown in FIG. 1049.

FIG. 1051 shows a nucleotide sequence (SEQ ID NO:1051) of a native sequence cDNA, wherein SEQ ID NO:1051 is a clone designated herein as “DNA328771”.

FIG. 1052A-B shows a nucleotide sequence (SEQ ID NO:1052) of a native sequence PRO84852 cDNA, wherein SEQ ID NO:1052 is a clone designated herein as “DNA329252”.

FIG. 1053 shows the amino acid sequence (SEQ ID NO:1053) derived from the coding sequence of SEQ ID NO:1052 shown in FIG. 1052.

FIG. 1054 shows a nucleotide sequence (SEQ ID NO:1054) of a native sequence PRO37804 cDNA, wherein SEQ ID NO:1054 is a clone designated herein as “DNA227341”.

FIG. 1055 shows the amino acid sequence (SEQ ID NO:1055) derived from the coding sequence of SEQ ID NO:1054 shown in FIG. 1054.

FIG. 1056 shows a nucleotide sequence (SEQ ID NO:1056) of a native sequence PRO60536 cDNA, wherein SEQ ID NO:1056 is a clone designated herein as “DNA328774”.

FIG. 1057 shows the amino acid sequence (SEQ ID NO:1057) derived from the coding sequence of SEQ ID NO:1056 shown in FIG. 1056.

FIG. 1058 shows a nucleotide sequence (SEQ ID NO:1058) of a native sequence cDNA, wherein SEQ ID NO:1058 is a clone designated herein as “DNA151041”.

FIG. 1059 shows a nucleotide sequence (SEQ ID NO:1059) of a native sequence PRO83768 cDNA, wherein SEQ ID NO:1059 is a clone designated herein as “DNA327807”.

FIG. 1060 shows the amino acid sequence (SEQ ID NO:1060) derived from the coding sequence of SEQ ID NO:1059 shown in FIG. 1059.

FIG. 1061 shows a nucleotide sequence (SEQ ID NO:1061) of a native sequence PRO2 cDNA, wherein SEQ ID NO:1061 is a clone designated herein as “DNA51782”.

FIG. 1062 shows the amino acid sequence (SEQ ID NO:1062) derived from the coding sequence of SEQ ID NO:1061 shown in FIG. 1061.

FIG. 1063 shows a nucleotide sequence (SEQ ID NO:1063) of a native sequence PRO10498 cDNA, wherein SEQ ID NO:1063 is a clone designated herein as “DNA324224”.

FIG. 1064 shows the amino acid sequence (SEQ ID NO:1064) derived from the coding sequence of SEQ ID NO:1063 shown in FIG. 1063.

FIG. 1065 shows a nucleotide sequence (SEQ ID NO:1065) of a native sequence PRO84853 cDNA, wherein SEQ ID NO:1065 is a clone designated herein as “DNA329253”.

FIG. 1066 shows the amino acid sequence (SEQ ID NO:1066) derived from the coding sequence of SEQ ID NO:1065 shown in FIG. 1065.

FIG. 1067 shows a nucleotide sequence (SEQ ID NO:1067) of a native sequence PRO84854 cDNA, wherein SEQ ID NO:1067 is a clone designated herein as “DNA329254”.

FIG. 1068 shows the amino acid sequence (SEQ ID NO:1068) derived from the coding sequence of SEQ ID NO:1067 shown in FIG. 1067.

FIG. 1069A-B shows a nucleotide sequence (SEQ ID NO:1069) of a native sequence PRO60550 cDNA, wherein SEQ ID NO:1069 is a clone designated herein as “DNA272292”.

FIG. 1070 shows the amino acid sequence (SEQ ID NO:1070) derived from the coding sequence of SEQ ID NO:1069 shown in FIG. 1069.

FIG. 1071 shows a nucleotide sequence (SEQ ID NO:1071) of a native sequence PRO84855 cDNA, wherein SEQ ID NO:1071 is a clone designated herein as “DNA329255”.

FIG. 1072 shows the amino acid sequence (SEQ ID NO:1072) derived from the coding sequence of SEQ ID NO:1071 shown in FIG. 1071.

FIG. 1073A-B shows a nucleotide sequence (SEQ ID NO:1073) of a native sequence PRO84856 cDNA, wherein SEQ ID NO:1073 is a clone designated herein as “DNA329256”.

FIG. 1074 shows the amino acid sequence (SEQ ID NO:1074) derived from the coding sequence of SEQ ID NO:1073 shown in FIG. 1073.

FIG. 1075 shows a nucleotide sequence (SEQ ID NO:1075) of a native sequence PRO2398 cDNA, wherein SEQ ID NO:1075 is a clone designated herein as “DNA88530”.

FIG. 1076 shows the amino acid sequence (SEQ ID NO:1076) derived from the coding sequence of SEQ ID NO:1075 shown in FIG. 1075.

FIG. 1077 shows a nucleotide sequence (SEQ ID NO:1077) of a native sequence PRO2634 cDNA, wherein SEQ ID NO:1077 is a clone designated herein as “DNA88054”.

FIG. 1078 shows the amino acid sequence (SEQ ID NO:1078) derived from the coding sequence of SEQ ID NO:1077 shown in FIG. 1077.

FIG. 1079 shows a nucleotide sequence (SEQ ID NO:1079) of a native sequence PRO83772 cDNA, wherein SEQ ID NO:1079 is a clone designated herein as “DNA327811”.

FIG. 1080 shows the amino acid sequence (SEQ ID NO:1080) derived from the coding sequence of SEQ ID NO:1079 shown in FIG. 1079.

FIG. 1081 shows a nucleotide sequence (SEQ ID NO:1081) of a native sequence PRO12564 cDNA, wherein SEQ ID NO:1081 is a clone designated herein as “DNA150971”.

FIG. 1082 shows the amino acid sequence (SEQ ID NO:1082) derived from the coding sequence of SEQ ID NO:1081 shown in FIG. 1081.

FIG. 1083A-B shows a nucleotide sequence (SEQ ID NO:1083) of a native sequence PRO84857 cDNA, wherein SEQ ID NO:1083 is a clone designated herein as “DNA329257”.

FIG. 1084 shows the amino acid sequence (SEQ ID NO:1084) derived from the coding sequence of SEQ ID NO:1083 shown in FIG. 1083.

FIG. 1085 shows a nucleotide sequence (SEQ ID NO:1085) of a native sequence PRO38069 cDNA, wherein SEQ ID NO:1085 is a clone designated herein as “DNA227606”.

FIG. 1086 shows the amino acid sequence (SEQ ID NO:1086) derived from the coding sequence of SEQ ID NO:1085 shown in FIG. 1085.

FIG. 1087 shows a nucleotide sequence (SEQ ID NO:1087) of a native sequence PRO71203 cDNA, wherein SEQ ID NO:1087 is a clone designated herein as “DNA304791”.

FIG. 1088 shows the amino acid sequence (SEQ ID NO:1088) derived from the coding sequence of SEQ ID NO:1087 shown in FIG. 1087.

FIG. 1089 shows a nucleotide sequence (SEQ ID NO:1089) of a native sequence PRO10586 cDNA, wherein SEQ ID NO:1089 is a clone designated herein as “DNA329258”.

FIG. 1090 shows the amino acid sequence (SEQ ID NO:1090) derived from the coding sequence of SEQ ID NO:1089 shown in FIG. 1089.

FIG. 1091 shows a nucleotide sequence (SEQ ID NO:1091) of a native sequence PRO34267 cDNA, wherein SEQ ID NO:1091 is a clone designated herein as “DNA216515”.

FIG. 1092 shows the amino acid sequence (SEQ ID NO:1092) derived from the coding sequence of SEQ ID NO:1091 shown in FIG. 1091.

FIG. 1093A-B shows a nucleotide sequence (SEQ ID NO:1093) of a native sequence PRO85430 cDNA, wherein SEQ ID NO:1093 is a clone designated herein as “DNA328784”.

FIG. 1094 shows the amino acid sequence (SEQ ID NO:1094) derived from the coding sequence of SEQ ID NO:1093 shown in FIG. 1093.

FIG. 1095 shows a nucleotide sequence (SEQ ID NO:1095) of a native sequence PRO1573 cDNA, wherein SEQ ID NO:1095 is a clone designated herein as “DNA327817”.

FIG. 1096 shows the amino acid sequence (SEQ ID NO:1096) derived from the coding sequence of SEQ ID NO:1095 shown in FIG. 1095.

FIG. 1097 shows a nucleotide sequence (SEQ ID NO:1097) of a native sequence PRO12646 cDNA, wherein SEQ ID NO:1097 is a clone designated herein as “DNA151222”.

FIG. 1098 shows the amino acid sequence (SEQ ID NO:1098) derived from the coding sequence of SEQ ID NO:1097 shown in FIG. 1097.

FIG. 1099 shows a nucleotide sequence (SEQ ID NO:1099) of a native sequence cDNA, wherein SEQ ID NO:1099 is a clone designated herein as “DNA329259”.

FIG. 1100 shows a nucleotide sequence (SEQ ID NO:1100) of a native sequence PRO83851 cDNA, wherein SEQ ID NO:1100 is a clone designated herein as “DNA327916”.

FIG. 1101 shows the amino acid sequence (SEQ ID NO:1101) derived from the coding sequence of SEQ ID NO:1100 shown in FIG. 1100.

FIG. 1102A-B shows a nucleotide sequence (SEQ ID NO:1102) of a native sequence PRO84858 cDNA, wherein SEQ ID NO:1102 is a clone designated herein as “DNA329260”.

FIG. 1103 shows the amino acid sequence (SEQ ID NO:1103) derived from the coding sequence of SEQ ID NO:1102 shown in FIG. 1102.

FIG. 1104A-B shows a nucleotide sequence (SEQ ID NO:1104) of a native sequence PRO84859 cDNA, wherein SEQ ID NO:1104 is a clone designated herein as “DNA329261”.

FIG. 1105 shows the amino acid sequence (SEQ ID NO:1105) derived from the coding sequence of SEQ ID NO:1104 shown in FIG. 1104.

FIG. 1106 shows a nucleotide sequence (SEQ ID NO:1106) of a native sequence PRO1721 cDNA, wherein SEQ ID NO:1106 is a clone designated herein as “DNA328799”.

FIG. 1107 shows the amino acid sequence (SEQ ID NO:1107) derived from the coding sequence of SEQ ID NO:1106 shown in FIG. 1106.

FIG. 1108 shows a nucleotide sequence (SEQ ID NO:1108) of a native sequence PRO84860 cDNA, wherein SEQ ID NO:1108 is a clone designated herein as “DNA329262”.

FIG. 1109 shows the amino acid sequence (SEQ ID NO:1109) derived from the coding sequence of SEQ ID NO:1108 shown in FIG. 1108.

FIG. 1110 shows a nucleotide sequence (SEQ ID NO:1110) of a native sequence PRO84861 cDNA, wherein SEQ ID NO:1110 is a clone designated herein as “DNA329263”.

FIG. 1111 shows the amino acid sequence (SEQ ID NO:1111) derived from the coding sequence of SEQ ID NO:1110 shown in FIG. 1110.

FIG. 1112 shows a nucleotide sequence (SEQ ID NO:1112) of a native sequence PRO60325 cDNA, wherein SEQ ID NO:1112 is a clone designated herein as “DNA326136”.

FIG. 1113 shows the amino acid sequence (SEQ ID NO:1113) derived from the coding sequence of SEQ ID NO:1112 shown in FIG. 1112.

FIG. 1114A-B shows a nucleotide sequence (SEQ ID NO:1114) of a native sequence cDNA, wherein SEQ ID NO:1114 is a clone designated herein as “DNA327827”.

FIG. 1115 shows a nucleotide sequence (SEQ ID NO:1115) of a native sequence PRO2178 cDNA, wherein SEQ ID NO:1115 is a clone designated herein as “DNA88121”.

FIG. 1116 shows the amino acid sequence (SEQ ID NO:1116) derived from the coding sequence of SEQ ID NO:1115 shown in FIG. 1115.

FIG. 1117A-B shows a nucleotide sequence (SEQ ID NO:1117) of a native sequence PRO12587 cDNA, wherein SEQ ID NO:1117 is a clone designated herein as “DNA151045”.

FIG. 1118 shows the amino acid sequence (SEQ ID NO:1118) derived from the coding sequence of SEQ ID NO:1117 shown in FIG. 1117.

FIG. 1119 shows a nucleotide sequence (SEQ ID NO:1119) of a native sequence PRO9819 cDNA, wherein SEQ ID NO:1119 is a clone designated herein as “DNA325174”.

FIG. 1120 shows the amino acid sequence (SEQ ID NO:1120) derived from the coding sequence of SEQ ID NO:1119 shown in FIG. 1119.

FIG. 1121 shows a nucleotide sequence (SEQ ID NO:1121) of a native sequence cDNA, wherein SEQ ID NO:1121 is a clone designated herein as “DNA329264”.

FIG. 1122 shows a nucleotide sequence (SEQ ID NO:1122) of a native sequence PRO84547 cDNA, wherein SEQ ID NO:1122 is a clone designated herein as “DNA328805”.

FIG. 1123 shows the amino acid sequence (SEQ ID NO:1123) derived from the coding sequence of SEQ ID NO:1122 shown in FIG. 1122.

FIG. 1124 shows a nucleotide sequence (SEQ ID NO:1124) of a native sequence PRO66288 cDNA, wherein SEQ ID NO:1124 is a clone designated herein as “DNA281449”.

FIG. 1125 shows the amino acid sequence (SEQ ID NO:1125) derived from the coding sequence of SEQ ID NO:1124 shown in FIG. 1124.

FIG. 1126 shows a nucleotide sequence (SEQ ID NO:1126) of a native sequence cDNA, wherein SEQ ID NO:1126 is a clone designated herein as “DNA329265”.

FIG. 1127 shows a nucleotide sequence (SEQ ID NO:1127) of a native sequence PRO12845 cDNA, wherein SEQ ID NO:1127 is a clone designated herein as “DNA329266”.

FIG. 1128 shows the amino acid sequence (SEQ ID NO:1128) derived from the coding sequence of SEQ ID NO:1127 shown in FIG. 1127.

FIG. 1129 shows a nucleotide sequence (SEQ ID NO:1129) of a native sequence PRO69518 cDNA, wherein SEQ ID NO:1129 is a clone designated herein as “DNA287243”.

FIG. 1130 shows the amino acid sequence (SEQ ID NO:1130) derived from the coding sequence of SEQ ID NO:1129 shown in FIG. 1129.

FIG. 1131 shows a nucleotide sequence (SEQ ID NO:1131) of a native sequence PRO2274 cDNA, wherein SEQ ID NO:1131 is a clone designated herein as “DNA88296”.

FIG. 1132 shows the amino acid sequence (SEQ ID NO:1132) derived from the coding sequence of SEQ ID NO:1131 shown in FIG. 1131.

FIG. 1133 shows a nucleotide sequence (SEQ ID NO:1133) of a native sequence PRO58102 cDNA, wherein SEQ ID NO:1133 is a clone designated herein as “DNA269692”.

FIG. 1134 shows the amino acid sequence (SEQ ID NO:1134) derived from the coding sequence of SEQ ID NO:1133 shown in FIG. 1133.

FIG. 1135 shows a nucleotide sequence (SEQ ID NO:1135) of a native sequence PRO21800 cDNA, wherein SEQ ID NO:1135 is a clone designated herein as “DNA188275”.

FIG. 1136 shows the amino acid sequence (SEQ ID NO:1136) derived from the coding sequence of SEQ ID NO:1135 shown in FIG. 1135.

FIG. 1137A-B shows a nucleotide sequence (SEQ ID NO:1137) of a native sequence PRO81897 cDNA, wherein SEQ ID NO: 1137 is a clone designated herein as “DNA325359”.

FIG. 1138 shows the amino acid sequence (SEQ ID NO:1138) derived from the coding sequence of SEQ ID NO:1137 shown in FIG. 1137.

FIG. 1139 shows a nucleotide sequence (SEQ ID NO:1139) of a native sequence cDNA, wherein SEQ ID NO:1139 is a clone designated herein as “DNA329267”.

FIG. 1140 shows a nucleotide sequence (SEQ ID NO:1140) of a native sequence cDNA, wherein SEQ ID NO:1140 is a clone designated herein as “DNA270839”.

FIG. 1141 shows a nucleotide sequence (SEQ ID NO:1141) of a native sequence PRO62882 cDNA, wherein SEQ ID NO:1141 is a clone designated herein as “DNA275181”.

FIG. 1142 shows the amino acid sequence (SEQ ID NO:1142) derived from the coding sequence of SEQ ID NO:1141 shown in FIG. 1141.

FIG. 1143A-C shows a nucleotide sequence (SEQ ID NO:1143) of a native sequence PRO84864 cDNA, wherein SEQ ID NO:1143 is a clone designated herein as “DNA329268”.

FIG. 1144 shows the amino acid sequence (SEQ ID NO:1144) derived from the coding sequence of SEQ ID NO:1143 shown in FIG. 1143.

FIG. 1145A-B shows a nucleotide sequence (SEQ ID NO:1145) of a native sequence PRO84865 cDNA, wherein SEQ ID NO:1145 is a clone designated herein as “DNA329269”.

FIG. 1146 shows the amino acid sequence (SEQ ID NO:1146) derived from the coding sequence of SEQ ID NO:1145 shown in FIG. 1145.

FIG. 1147 shows a nucleotide sequence (SEQ ID NO:1147) of a native sequence PRO84866 cDNA, wherein SEQ ID NO:1147 is a clone designated herein as “DNA329270”.

FIG. 1148 shows the amino acid sequence (SEQ ID NO:1148) derived from the coding sequence of SEQ ID NO:1147 shown in FIG. 1147.

FIG. 1149 shows a nucleotide sequence (SEQ ID NO:1149) of a native sequence PRO38457 cDNA, wherein SEQ ID NO:1149 is a clone designated herein as “DNA227994”.

FIG. 1150 shows the amino acid sequence (SEQ ID NO:1150) derived from the coding sequence of SEQ ID NO:1149 shown in FIG. 1149.

FIG. 1151 shows a nucleotide sequence (SEQ ID NO:1151) of a native sequence PRO84867 cDNA, wherein SEQ ID NO:1151 is a clone designated herein as “DNA329271”.

FIG. 1152 shows the amino acid sequence (SEQ ID NO:1152) derived from the coding sequence of SEQ ID NO:1151 shown in FIG. 1151.

FIG. 1153 shows a nucleotide sequence (SEQ ID NO:1153) of a native sequence PRO1869 cDNA, wherein SEQ ID NO:1153 is a clone designated herein as “DNA325832”.

FIG. 1154 shows the amino acid sequence (SEQ ID NO:1154) derived from the coding sequence of SEQ ID NO:1153 shown in FIG. 1153.

FIG. 1155A-B shows a nucleotide sequence (SEQ ID NO:1155) of a native sequence PRO2834 cDNA, wherein SEQ ID NO:1155 is a clone designated herein as “DNA327847”.

FIG. 1156 shows the amino acid sequence (SEQ ID NO:1156) derived from the coding sequence of SEQ ID NO:1155 shown in FIG. 1155.

FIG. 1157 shows a nucleotide sequence (SEQ ID NO:1157) of a native sequence PRO2834 cDNA, wherein SEQ ID NO:1157 is a clone designated herein as “DNA88541”.

FIG. 1158 shows the amino acid sequence (SEQ ID NO:1158) derived from the coding sequence of SEQ ID NO:1157 shown in FIG. 1157.

FIG. 1159 shows a nucleotide sequence (SEQ ID NO:1159) of a native sequence PRO83794 cDNA, wherein SEQ ID NO:1159 is a clone designated herein as “DNA327849”.

FIG. 1160 shows the amino acid sequence (SEQ ID NO:1160) derived from the coding sequence of SEQ ID NO:1159 shown in FIG. 1159.

FIG. 1161 shows a nucleotide sequence (SEQ ID NO:1161) of a native sequence PRO82287 cDNA, wherein SEQ ID NO:1161 is a clone designated herein as “DNA325821”.

FIG. 1162 shows the amino acid sequence (SEQ ID NO:1162) derived from the coding sequence of SEQ ID NO:1161 shown in FIG. 1161.

FIG. 1163 shows a nucleotide sequence (SEQ ID NO:1163) of a native sequence PRO84557 cDNA, wherein SEQ ID NO:1163 is a clone designated herein as “DNA328819”.

FIG. 1164 shows the amino acid sequence (SEQ ID NO:1164) derived from the coding sequence of SEQ ID NO:1163 shown in FIG. 1163.

FIG. 1165 shows a nucleotide sequence (SEQ ID NO:1165) of a native sequence PRO82331 cDNA, wherein SEQ ID NO:1165 is a clone designated herein as “DNA325873”.

FIG. 1166 shows the amino acid sequence (SEQ ID NO:1166) derived from the coding sequence of SEQ ID NO:1165 shown in FIG. 1165.

FIG. 1167 shows a nucleotide sequence (SEQ ID NO:1167) of a native sequence PRO84561 cDNA, wherein SEQ ID NO:1167 is a clone designated herein as “DNA328823”.

FIG. 1168 shows the amino acid sequence (SEQ ID NO:1168) derived from the coding sequence of SEQ ID NO:1167 shown in FIG. 1167.

FIG. 1169 shows a nucleotide sequence (SEQ ID NO:1169) of a native sequence PRO37635 cDNA, wherein SEQ ID NO:1169 is a clone designated herein as “DNA227172”.

FIG. 1170 shows the amino acid sequence (SEQ ID NO:1170) derived from the coding sequence of SEQ ID NO:1169 shown in FIG. 1169.

FIG. 1171 shows a nucleotide sequence (SEQ ID NO:1171) of a native sequence PRO84868 cDNA, wherein SEQ ID NO:1171 is a clone designated herein as “DNA329272”.

FIG. 1172 shows the amino acid sequence (SEQ ID NO:1172) derived from the coding sequence of SEQ ID NO:1171 shown in FIG. 1171

FIG. 1173 shows a nucleotide sequence (SEQ ID NO:1173) of a native sequence PRO82223 cDNA, wherein SEQ ID NO:1173 is a clone designated herein as “DNA327853”.

FIG. 1174 shows the amino acid sequence (SEQ ID NO:1174) derived from the coding sequence of SEQ ID NO:1173 shown in FIG. 1173.

FIG. 1175 shows a nucleotide sequence (SEQ ID NO:1175) of a native sequence PRO84869 cDNA, wherein SEQ ID NO:1175 is a clone designated herein as “DNA329273”.

FIG. 1176 shows the amino acid sequence (SEQ ID NO:1176) derived from the coding sequence of SEQ ID NO:1175 shown in FIG. 1175.

FIG. 1177 shows a nucleotide sequence (SEQ ID NO:1177) of a native sequence PRO84870 cDNA, wherein SEQ ID NO:1177 is a clone designated herein as “DNA329274”.

FIG. 1178 shows the amino acid sequence (SEQ ID NO:1178) derived from the coding sequence of SEQ ID NO:1177 shown in FIG. 1177.

FIG. 1179 shows a nucleotide sequence (SEQ ID NO:1179) of a native sequence PRO82813 cDNA, wherein SEQ ID NO:1179 is a clone designated herein as “DNA326428”.

FIG. 1180 shows the amino acid sequence (SEQ ID NO:1180) derived from the coding sequence of SEQ ID NO:1179 shown in FIG. 1179.

FIG. 1181 shows a nucleotide sequence (SEQ ID NO:1181) of a native sequence PRO37681 cDNA, wherein SEQ ID NO:1181 is a clone designated herein as “DNA227218”.

FIG. 1182 shows the amino acid sequence (SEQ ID NO:1182) derived from the coding sequence of SEQ ID NO:1181 shown in FIG. 1181.

FIG. 1183 shows a nucleotide sequence (SEQ ID NO:1183) of a native sequence PRO233 cDNA, wherein SEQ ID NO:1183 is a clone designated herein as “DNA328831”.

FIG. 1184 shows the amino acid sequence (SEQ ID NO:1184) derived from the coding sequence of SEQ ID NO:1183 shown in FIG. 1183.

FIG. 1185 shows a nucleotide sequence (SEQ ID NO:1185) of a native sequence PRO84569 cDNA, wherein SEQ ID NO:1185 is a clone designated herein as “DNA328833”.

FIG. 1186 shows the amino acid sequence (SEQ ID NO:1186) derived from the coding sequence of SEQ ID NO:1185 shown in FIG. 1186.

FIG. 1187 shows a nucleotide sequence (SEQ ID NO:) of a native sequence PRO84572 cDNA, wherein SEQ ID NO:1187 is a clone designated herein as “DNA328836”.

FIG. 1188 shows the amino acid sequence (SEQ ID NO:1188) derived from the coding sequence of SEQ ID NO:1187 shown in FIG. 1187.

FIG. 1189 shows a nucleotide sequence (SEQ ID NO:1189) of a native sequence PRO12342 cDNA, wherein SEQ ID NO:1189 is a clone designated herein as “DNA329275”.

FIG. 1190 shows the amino acid sequence (SEQ ID NO:1190) derived from the coding sequence of SEQ ID NO:1189 shown in FIG. 1189.

FIG. 1191 shows a nucleotide sequence (SEQ ID NO:1191) of a native sequence PRO12104 cDNA, wherein SEQ ID NO:1191 is a clone designated herein as “DNA329276”.

FIG. 1192 shows the amino acid sequence (SEQ ID NO:1192) derived from the coding sequence of SEQ ID NO:1191 shown in FIG. 1191.

FIG. 1193 shows a nucleotide sequence (SEQ ID NO:1193) of a native sequence PRO84575 cDNA, wherein SEQ ID NO:1193 is a clone designated herein as “DNA328841”.

FIG. 1194 shows the amino acid sequence (SEQ ID NO:1194) derived from the coding sequence of SEQ ID NO:1193 shown in FIG. 1193.

FIG. 1195 shows a nucleotide sequence (SEQ ID NO:1195) of a native sequence PRO6241 cDNA, wherein SEQ ID NO:1195 is a clone designated herein as “DNA329277”.

FIG. 1196 shows the amino acid sequence (SEQ ID NO:1196) derived from the coding sequence of SEQ ID NO:1195 shown in FIG. 1195.

FIG. 1197 shows a nucleotide sequence (SEQ ID NO:1197) of a native sequence PRO84871 cDNA, wherein SEQ ID NO:1197 is a clone designated herein as “DNA329278”.

FIG. 1198 shows the amino acid sequence (SEQ ID NO:1198) derived from the coding sequence of SEQ ID NO:1197 shown in FIG. 1197.

FIG. 1199 shows a nucleotide sequence (SEQ ID NO:1199) of a native sequence PRO62669 cDNA, wherein SEQ ID NO:1199 is a clone designated herein as “DNA274930”.

FIG. 1200 shows the amino acid sequence (SEQ ID NO:1200) derived from the coding sequence of SEQ ID NO:1199 shown in FIG. 1199.

FIG. 1201 shows a nucleotide sequence (SEQ ID NO:1201) of a native sequence PRO84872 cDNA, wherein SEQ ID NO:1201 is a clone designated herein as “DNA329279”.

FIG. 1202 shows the amino acid sequence (SEQ ID NO:1202) derived from the coding sequence of SEQ ID NO:1201 shown in FIG. 1201.

FIG. 1203 shows a nucleotide sequence (SEQ ID NO:1203) of a native sequence PRO49837 cDNA, wherein SEQ ID NO:1203 is a clone designated herein as “DNA254739”.

FIG. 1204 shows the amino acid sequence (SEQ ID NO:1204) derived from the coding sequence of SEQ ID NO:1203 shown in FIG. 1203.

FIG. 1205 shows a nucleotide sequence (SEQ ID NO:1205) of a native sequence PRO84873 cDNA, wherein SEQ ID NO:1205 is a clone designated herein as “DNA329280”.

FIG. 1206 shows the amino acid sequence (SEQ ID NO:1206) derived from the coding sequence of SEQ ID NO:1205 shown in FIG. 1205.

FIG. 1207 shows a nucleotide sequence (SEQ ID NO:1207) of a native sequence PRO83800 cDNA, wherein SEQ ID NO:1207 is a clone designated herein as “DNA327858”.

FIG. 1208 shows the amino acid sequence (SEQ ID NO:1208) derived from the coding sequence of SEQ ID NO:1207 shown in FIG. 1207.

FIG. 1209 shows a nucleotide sequence (SEQ ID NO:1209) of a native sequence PRO84581 cDNA, wherein SEQ ID NO:1209 is a clone designated herein as “DNA328850”.

FIG. 1210 shows the amino acid sequence (SEQ ID NO:1210) derived from the coding sequence of SEQ ID NO:1209 shown in FIG. 1209.

FIG. 1211 shows a nucleotide sequence (SEQ ID NO:1211) of a native sequence PRO61257 cDNA, wherein SEQ ID NO:1211 is a clone designated herein as “DNA273230”.

FIG. 1212 shows the amino acid sequence (SEQ ID NO:1212) derived from the coding sequence of SEQ ID NO:1211 shown in FIG. 1211.

FIG. 1213 shows a nucleotide sequence (SEQ ID NO:1213) of a native sequence PRO82325 cDNA, wherein SEQ ID NO:1213 is a clone designated herein as “DNA325866”.

FIG. 1214 shows the amino acid sequence (SEQ ID NO:1214) derived from the coding sequence of SEQ ID NO:1213 shown in FIG. 1213.

FIG. 1215 shows a nucleotide sequence (SEQ ID NO:1215) of a native sequence PRO84874 cDNA, wherein SEQ ID NO:1215 is a clone designated herein as “DNA329281”.

FIG. 1216 shows the amino acid sequence (SEQ ID NO:1216) derived from the coding sequence of SEQ ID NO:1215 shown in FIG. 1215.

FIG. 1217 shows a nucleotide sequence (SEQ ID NO:1217) of a native sequence PRO84875 cDNA, wherein SEQ ID NO:1217 is a clone designated herein as “DNA329282”.

FIG. 1218 shows the amino acid sequence (SEQ ID NO:1218) derived from the coding sequence of SEQ ID NO:1217 shown in FIG. 1217.

FIG. 1219 shows a nucleotide sequence (SEQ ID NO:1219) of a native sequence PRO61003 cDNA, wherein SEQ ID NO:1219 is a clone designated herein as “DNA272918”.

FIG. 1220 shows the amino acid sequence (SEQ ID NO:1220) derived from the coding sequence of SEQ ID NO:1219 shown in FIG. 1219.

FIG. 1221 shows a nucleotide sequence (SEQ ID NO:1221) of a native sequence PRO84876 cDNA, wherein SEQ ID NO:1221 is a clone designated herein as “DNA329283”.

FIG. 1222 shows the amino acid sequence (SEQ ID NO:1222) derived from the coding sequence of SEQ ID NO:1221 shown in FIG. 1221.

FIG. 1223A-B shows a nucleotide sequence (SEQ ID NO:1223) of a native sequence PRO84877 cDNA, wherein SEQ ID NO:1223 is a clone designated herein as “DNA329284”.

FIG. 1224 shows the amino acid sequence (SEQ ID NO:1224) derived from the coding sequence of SEQ ID NO:1223 shown in FIG. 1223.

FIG. 1225 shows a nucleotide sequence (SEQ ID NO:1225) of a native sequence PRO84878 cDNA, wherein SEQ ID NO:1225 is a clone designated herein as “DNA329285”.

FIG. 1226 shows the amino acid sequence (SEQ ID NO:1226) derived from the coding sequence of SEQ ID NO:1225 shown in FIG. 1225.

FIG. 1227 shows a nucleotide sequence (SEQ ID NO:1227) of a native sequence PRO84592 cDNA; wherein SEQ ID NO:1227 is a clone designated herein as “DNA328864”.

FIG. 1228 shows the amino acid sequence (SEQ ID NO:1228) derived from the coding sequence of SEQ ID NO:1227 shown in FIG. 1227.

FIG. 1229 shows a nucleotide sequence (SEQ ID NO:1229) of a native sequence PRO224 cDNA, wherein SEQ ID NO:1229 is a clone designated herein as “DNA326550”.

FIG. 1230 shows the amino acid sequence (SEQ ID NO:1230) derived from the coding sequence of SEQ ID NO:1229 shown in FIG. 1229.

FIG. 1231 shows a nucleotide sequence (SEQ ID NO:1231) of a native sequence PRO69644 cDNA, wherein SEQ ID NO:1231 is a clone designated herein as “DNA329286”.

FIG. 1232 shows the amino acid sequence (SEQ ID NO:1232) derived from the coding sequence of SEQ ID NO:1231 shown in FIG. 1231.

FIG. 1233 shows a nucleotide sequence (SEQ ID NO:1233) of a native sequence PRO4929 cDNA, wherein SEQ ID NO:1233 is a clone designated herein as “DNA93548”.

FIG. 1234 shows the amino acid sequence (SEQ ID NO:1234) derived from the coding sequence of SEQ ID NO:1233 shown in FIG. 1233.

FIG. 1235 shows a nucleotide sequence (SEQ ID NO:1235) of a native sequence PRO84879 cDNA, wherein SEQ ID NO:1235 is a clone designated herein as “DNA329287”.

FIG. 1236 shows the amino acid sequence (SEQ ID NO:1236) derived from the coding sequence of SEQ ID NO:1235 shown in FIG. 1235.

FIG. 1237 shows a nucleotide sequence (SEQ ID NO:1237) of a native sequence PRO84880 cDNA, wherein SEQ ID NO:1237 is a clone designated herein as “DNA329288”.

FIG. 1238 shows the amino acid sequence (SEQ ID NO:1238) derived from the coding sequence of SEQ ID NO:1237 shown in FIG. 1237.

FIG. 1239 shows a nucleotide sequence (SEQ ID NO:1239) of a native sequence PRO38080 cDNA, wherein SEQ ID NO:1239 is a clone designated herein as “DNA227617”.

FIG. 1240 shows the amino acid sequence (SEQ ID NO:1240) derived from the coding sequence of SEQ ID NO:1239 shown in FIG. 1239.

FIG. 1241 shows a nucleotide sequence (SEQ ID NO:1241) of a native sequence PRO83216 cDNA, wherein SEQ ID NO:1241 is a clone designated herein as “DNA329289”.

FIG. 1242 shows the amino acid sequence (SEQ ID NO:1242) derived from the coding sequence of SEQ ID NO:1241 shown in FIG. 1241.

FIG. 1243 shows a nucleotide sequence (SEQ ID NO:1243) of a native sequence PRO4805 cDNA, wherein SEQ ID NO:1243 is a clone designated herein as “DNA327874”.

FIG. 1244 shows the amino acid sequence (SEQ ID NO:1244) derived from the coding sequence of SEQ ID NO:1243 shown in FIG. 1243.

FIG. 1245 shows a nucleotide sequence (SEQ ID NO:1245) of a native sequence PRO37096 cDNA, wherein SEQ ID NO:1245 is a clone designated herein as “DNA226633”.

FIG. 1246 shows the amino acid sequence (SEQ ID NO:1246) derived from the coding sequence of SEQ ID NO:1245 shown in FIG. 1245.

FIG. 1247 shows a nucleotide sequence (SEQ ID NO:1247) of a native sequence PRO69889 cDNA, wherein SEQ ID NO:1247 is a clone designated herein as “DNA304780”.

FIG. 1248 shows the amino acid sequence (SEQ ID NO:1248) derived from the coding sequence of SEQ ID NO:1247 shown in FIG. 1247.

FIG. 1249 shows a nucleotide sequence (SEQ ID NO:1249) of a native sequence PRO84881 cDNA, wherein SEQ ID NO:1249 is a clone designated herein as “DNA329290”.

FIG. 1250 shows the amino acid sequence (SEQ ID NO:1250) derived from the coding sequence of SEQ ID NO:1249 shown in FIG. 1249.

FIG. 1251 shows a nucleotide sequence (SEQ ID NO:1251) of a native sequence PRO1017 cDNA, wherein SEQ ID NO:1251 is a clone designated herein as “DNA329291”.

FIG. 1252 shows the amino acid sequence (SEQ ID NO:1252) derived from the coding sequence of SEQ ID NO:1251 shown in FIG. 1251.

FIG. 1253 shows a nucleotide sequence (SEQ ID NO:1253) of a native sequence PRO84712 cDNA, wherein SEQ ID NO:1253 is a clone designated herein as “DNA329050”.

FIG. 1254 shows the amino acid sequence (SEQ ID NO:1254) derived from the coding sequence of SEQ ID NO:1253 shown in FIG. 1253.

FIG. 1255 shows a nucleotide sequence (SEQ ID NO:1255) of a native sequence PRO37650 cDNA, wherein SEQ ID NO:1255 is a clone designated herein as “DNA227187”.

FIG. 1256 shows the amino acid sequence (SEQ ID NO:1256) derived from the coding sequence of SEQ ID NO:1255 shown in FIG. 1255.

FIG. 1257 shows a nucleotide sequence (SEQ ID NO:1257) of a native sequence PRO84882 cDNA, wherein SEQ ID NO:1257 is a clone designated herein as “DNA329292”.

FIG. 1258 shows the amino acid sequence (SEQ ID NO:1258) derived from the coding sequence of SEQ ID NO:1257 shown in FIG. 1257.

FIG. 1259 shows a nucleotide sequence (SEQ ID NO:1259) of a native sequence PRO84883 cDNA, wherein SEQ ID NO:1259 is a clone designated herein as “DNA329293”.

FIG. 1260 shows the amino acid sequence (SEQ ID NO:1260) derived from the coding sequence of SEQ ID NO:1259 shown in FIG. 1259.

FIG. 1261 shows a nucleotide sequence (SEQ ID NO:1261) of a native sequence PRO84884 cDNA, wherein SEQ ID NO:1261 is a clone designated herein as “DNA329294”.

FIG. 1262 shows the amino acid sequence (SEQ ID NO:1262) derived from the coding sequence of SEQ ID NO:1261 shown in FIG. 1261.

FIG. 1263 shows a nucleotide sequence (SEQ ID NO:1263) of a native sequence PRO69517 cDNA, wherein SEQ ID NO:1263 is a clone designated herein as “DNA287242”.

FIG. 1264 shows the amino acid sequence (SEQ ID NO:1264) derived from the coding sequence of SEQ ID NO:1263 shown in FIG. 1263.

FIG. 1265 shows a nucleotide sequence (SEQ ID NO:1265) of a native sequence PRO84885 cDNA, wherein SEQ ID NO:1265 is a clone designated herein as “DNA329295”.

FIG. 1266 shows the amino acid sequence (SEQ ID NO:1266) derived from the coding sequence of SEQ ID NO:1261 shown in FIG. 1261.

FIG. 1267 shows a nucleotide sequence (SEQ ID NO:1267) of a native sequence PRO83819 cDNA, wherein SEQ ID NO:1267 is a clone designated herein as “DNA327880”.

FIG. 1268 shows the amino acid sequence (SEQ ID NO:1268) derived from the coding sequence of SEQ ID NO:1267 shown in FIG. 1267.

FIG. 1269 shows a nucleotide sequence (SEQ ID NO:1269) of a native sequence PRO84617 cDNA, wherein SEQ ID NO:1269 is a clone designated herein as “DNA328894”.

FIG. 1270 shows the amino acid sequence (SEQ ID NO:1270) derived from the coding sequence of SEQ ID NO:1269 shown in FIG. 1269.

FIG. 1271 shows a nucleotide sequence (SEQ ID NO:1271) of a native sequence PRO51682 cDNA, wherein SEQ ID NO:1271 is a clone designated herein as “DNA256749”.

FIG. 1272 shows the amino acid sequence (SEQ ID NO:1272) derived from the coding sequence of SEQ ID NO:1271 shown in FIG. 1271.

FIG. 1273 shows a nucleotide sequence (SEQ ID NO:1273) of a native sequence PRO84886 cDNA, wherein SEQ ID NO:1273 is a clone designated herein as “DNA329296”.

FIG. 1274 shows the amino acid sequence (SEQ ID NO:1274) derived from the coding sequence of SEQ ID NO:1273 shown in FIG. 1273.

FIG. 1275 shows a nucleotide sequence (SEQ ID NO:1275) of a native sequence PRO82421 cDNA, wherein SEQ ID NO:1275 is a clone designated herein as “DNA325976”.

FIG. 1276 shows the amino acid sequence (SEQ ID NO:1276) derived from the coding sequence of SEQ ID NO:1275 shown in FIG. 1275.

FIG. 1277A-C shows a nucleotide sequence (SEQ ID NO:1277) of a native sequence PRO23303 cDNA, wherein SEQ ID NO:1277 is a clone designated herein as “DNA329297”.

FIG. 1278 shows the amino acid sequence (SEQ ID NO:1278) derived from the coding sequence of SEQ ID NO:1277 shown in FIG. 1277.

FIG. 1279 shows a nucleotide sequence (SEQ ID NO:1279) of a native sequence PRO50991 cDNA, wherein SEQ ID NO:1279 is a clone designated herein as “DNA255939”.

FIG. 1280 shows the amino acid sequence (SEQ ID NO:1280) derived from the coding sequence of SEQ ID NO:1279 shown in FIG. 1279.

FIG. 1281 shows a nucleotide sequence (SEQ ID NO:1281) of a native sequence PRO49625 cDNA, wherein SEQ ID NO:1281 is a clone designated herein as “DNA254518”.

FIG. 1282 shows the amino acid sequence (SEQ ID NO:1282) derived from the coding sequence of SEQ ID NO:1281 shown in FIG. 1281.

FIG. 1283A-B shows a nucleotide sequence (SEQ ID NO:1283) of a native sequence PRO84887 cDNA, wherein SEQ ID NO:1283 is a clone designated herein as “DNA329298”.

FIG. 1284 shows the amino acid sequence (SEQ ID NO:1284) derived from the coding sequence of SEQ ID NO:1283 shown in FIG. 1283.

FIG. 1285 shows a nucleotide sequence (SEQ ID NO:1285) of a native sequence PRO84622 cDNA, wherein SEQ ID NO:1285 is a clone designated herein as “DNA328901”.

FIG. 1286 shows the amino acid sequence (SEQ ID NO:1286) derived from the coding sequence of SEQ ID NO:1285 shown in FIG. 1285.

FIG. 1287A-B shows a nucleotide sequence (SEQ ID NO:1287) of a native sequence PRO37642 cDNA, wherein SEQ ID NO:1287 is a clone designated herein as “DNA227179”.

FIG. 1288 shows the amino acid sequence (SEQ ID NO:1288) derived from the coding sequence of SEQ ID NO:1287 shown in FIG. 1287.

FIG. 1289 shows a nucleotide sequence (SEQ ID NO:1289) of a native sequence PRO84888 cDNA, wherein SEQ ID NO:1289 is a clone designated herein as “DNA329299”.

FIG. 1290 shows the amino acid sequence (SEQ ID NO:1290) derived from the coding sequence of SEQ ID NO:1289 shown in FIG. 1289.

FIG. 1291 shows a nucleotide sequence (SEQ ID NO:1291) of a native sequence PRO84889 cDNA, wherein SEQ ID NO:1291 is a clone designated herein as “DNA329300”.

FIG. 1292 shows the amino acid sequence (SEQ ID NO:1292) derived from the coding sequence of SEQ ID NO:1291 shown in FIG. 1291.

FIG. 1293 shows a nucleotide sequence (SEQ ID NO:1293) of a native sequence PRO51671 cDNA, wherein SEQ ID NO:1293 is a clone designated herein as “DNA256737”.

FIG. 1294 shows the amino acid sequence (SEQ ID NO:1294) derived from the coding sequence of SEQ ID NO:1293 shown in FIG. 1293.

FIG. 1295 shows a nucleotide sequence (SEQ ID NO:1295) of a native sequence PRO84890 cDNA, wherein SEQ ID NO:1295 is a clone designated herein as “DNA329301”.

FIG. 1296 shows the amino acid sequence (SEQ ID NO:1296) derived from the coding sequence of SEQ ID NO:1295 shown in FIG. 1295.

FIG. 1297A-B shows a nucleotide sequence (SEQ ID NO:1297) of a native sequence PRO84891 cDNA, wherein SEQ ID NO:1297 is a clone designated herein as “DNA329302”.

FIG. 1298 shows the amino acid sequence (SEQ ID NO:1298) derived from the coding sequence of SEQ ID NO:1297 shown in FIG. 1297.

FIG. 1299 shows a nucleotide sequence (SEQ ID NO:1299) of a native sequence PRO84634 cDNA, wherein SEQ ID NO:1299 is a clone designated herein as “DNA328915”.

FIG. 1300 shows the amino acid sequence (SEQ ID NO:1300) derived from the coding sequence of SEQ ID NO:1299 shown in FIG. 1299.

FIG. 1301A-B shows a nucleotide sequence (SEQ ID NO:1301) of a native sequence PRO61987 cDNA, wherein SEQ ID NO:1301 is a clone designated herein as “DNA274044”.

FIG. 1302 shows the amino acid sequence (SEQ ID NO:1302) derived from the coding sequence of SEQ ID NO:1301 shown in FIG. 1301.

FIG. 1303 shows a nucleotide sequence (SEQ ID NO:1303) of a native sequence PRO84892 cDNA, wherein SEQ ID NO:1303 is a clone designated herein as “DNA329303”.

FIG. 1304 shows the amino acid sequence (SEQ ID NO:1304) derived from the coding sequence of SEQ ID NO:1303 shown in FIG. 1303.

FIG. 1305 shows a nucleotide sequence (SEQ ID NO:1305) of a native sequence PRO69564 cDNA, wherein SEQ ID NO:1305 is a clone designated herein as “DNA287295”.

FIG. 1306 shows the amino acid sequence (SEQ ID NO:1306) derived from the coding sequence of SEQ ID NO:1305 shown in FIG. 1305.

FIG. 1307 shows a nucleotide sequence (SEQ ID NO:1307) of a native sequence PRO84893 cDNA, wherein SEQ ID NO:1307 is a clone designated herein as “DNA329304”.

FIG. 1308 shows the amino acid sequence (SEQ ID NO:1308) derived from the coding sequence of SEQ ID NO:1307 shown in FIG. 1307.

FIG. 1309 shows a nucleotide sequence (SEQ ID NO:1309) of a native sequence PRO83839 cDNA, wherein SEQ ID NO:1309 is a clone designated herein as “DNA327904”.

FIG. 1310 shows the amino acid sequence (SEQ ID NO:1310) derived from the coding sequence of SEQ ID NO:1309 shown in FIG. 1309.

FIG. 1311 shows a nucleotide sequence (SEQ ID NO:1311) of a native sequence PRO69472 cDNA, wherein SEQ ID NO:1311 is a clone designated herein as “DNA287186”.

FIG. 1312 shows the amino acid sequence (SEQ ID NO:1312) derived from the coding sequence of SEQ ID NO:1311 shown in FIG. 1311.

FIG. 1313 shows a nucleotide sequence (SEQ ID NO:1313) of a native sequence PRO84894 cDNA, wherein SEQ ID NO:1313 is a clone designated herein as “DNA329305”.

FIG. 1314 shows the amino acid sequence (SEQ ID NO:1314) derived from the coding sequence of SEQ ID NO:1313 shown in FIG. 1313.

FIG. 1315 shows a nucleotide sequence (SEQ ID NO:1315) of a native sequence PRO84895 cDNA, wherein SEQ ID NO:1315 is a clone designated herein as “DNA329306”.

FIG. 1316 shows the amino acid sequence (SEQ ID NO:1316) derived from the coding sequence of SEQ ID NO:1315 shown in FIG. 1315.

FIG. 1317 shows a nucleotide sequence (SEQ ID NO:1317) of a native sequence PRO84896 cDNA, wherein SEQ ID NO:1317 is a clone designated herein as “DNA329307”.

FIG. 1318 shows the amino acid sequence (SEQ ID NO:1318) derived from the coding sequence of SEQ ID NO:1317 shown in FIG. 1317.

FIG. 1319 shows a nucleotide sequence (SEQ ID NO:1319) of a native sequence PRO80512 cDNA, wherein SEQ ID NO:1319 is a clone designated herein as “DNA323756”.

FIG. 1320 shows the amino acid sequence (SEQ ID NO:1320) derived from the coding sequence of SEQ ID NO:1319 shown in FIG. 1319.

FIG. 1321 A-B shows a nucleotide sequence (SEQ ID NO:1321) of a native sequence PRO84897 cDNA, wherein SEQ ID NO:1321 is a clone designated herein as “DNA329308”.

FIG. 1322 shows the amino acid sequence (SEQ ID NO:1322) derived from the coding sequence of SEQ ID NO:1321 shown in FIG. 1321.

FIG. 1323 shows a nucleotide sequence (SEQ ID NO:1323) of a native sequence PRO84898 cDNA, wherein SEQ ID NO:1323 is a clone designated herein as “DNA329309”.

FIG. 1324 shows the amino acid sequence (SEQ ID NO:1324) derived from the coding sequence of SEQ ID NO:1323 shown in FIG. 1323.

FIG. 1325 shows a nucleotide sequence (SEQ ID NO:1325) of a native sequence PRO70111 cDNA, wherein SEQ ID NO:1325 is a clone designated herein as “DNA288247”.

FIG. 1326 shows the amino acid sequence (SEQ ID NO:1326) derived from the coding sequence of SEQ ID NO:1325 shown in FIG. 1325.

FIG. 1327 shows a nucleotide sequence (SEQ ID NO:1327) of a native sequence PRO23253 cDNA, wherein SEQ ID NO:1327 is a clone designated herein as “DNA329078”.

FIG. 1328 shows the amino acid sequence (SEQ ID NO:1328) derived from the coding sequence of SEQ ID NO:1327 shown in FIG. 1327.

FIG. 1329 shows a nucleotide sequence (SEQ ID NO:1329) of a native sequence PRO36690 cDNA, wherein SEQ ID NO:1329 is a clone designated herein as “DNA226227”.

FIG. 1330 shows the amino acid sequence (SEQ ID NO:1330) derived from the coding sequence of SEQ ID NO:1329 shown in FIG. 1329.

FIG. 1331 shows a nucleotide sequence (SEQ ID NO:1331) of a native sequence PRO34298 cDNA, wherein SEQ ID NO:1331 is a clone designated herein as “DNA217256”.

FIG. 1332 shows the amino acid sequence (SEQ ID NO:1332) derived from the coding sequence of SEQ ID NO:1331 shown in FIG. 1331.

FIG. 1333 shows a nucleotide sequence (SEQ ID NO:1333) of a native sequence PRO84899 cDNA, wherein SEQ ID NO:1333 is a clone designated herein as “DNA329310”.

FIG. 1334 shows the amino acid sequence (SEQ ID NO:1334) derived from the coding sequence of SEQ ID NO:1333 shown in FIG. 1333.

FIG. 1335 shows a nucleotide sequence (SEQ ID NO:1335) of a native sequence PRO84659 cDNA, wherein SEQ ID NO:1335 is a clone designated herein as “DNA328948”.

FIG. 1336 shows the amino acid sequence (SEQ ID NO:1336) derived from the coding sequence of SEQ ID NO:1335 shown in FIG. 1335.

FIG. 1337 shows a nucleotide sequence (SEQ ID NO:1337) of a native sequence PRO52290 cDNA, wherein SEQ ID NO:1337 is a clone designated herein as “DNA257738”.

FIG. 1338 shows the amino acid sequence (SEQ ID NO:1338) derived from the coding sequence of SEQ ID NO:1337 shown in FIG. 1337.

FIG. 1339 shows a nucleotide sequence (SEQ ID NO:1339) of a native sequence PRO84900 cDNA, wherein SEQ ID NO:1339 is a clone designated herein as “DNA3293 11”.

FIG. 1340 shows the amino acid sequence (SEQ ID NO:1340) derived from the coding sequence of SEQ ID NO:1339 shown in FIG. 1339.

FIG. 1341 shows a nucleotide sequence (SEQ ID NO:1341) of a native sequence PRO84901 cDNA, wherein SEQ ID NO:1341 is a clone designated herein as “DNA329312”.

FIG. 1342 shows the amino acid sequence (SEQ ID NO:1342) derived from the coding sequence of SEQ ID NO:1341 shown in FIG. 1341.

FIG. 1343 shows a nucleotide sequence (SEQ ID NO:1343) of a native sequence PRO70013 cDNA, wherein SEQ ID NO:1343 is a clone designated herein as “DNA288250”.

FIG. 1344 shows the amino acid sequence (SEQ ID NO:1344) derived from the coding sequence of SEQ ID NO:1343 shown in FIG. 1343.

FIG. 1345 shows a nucleotide sequence (SEQ ID NO:1345) of a native sequence PRO83944 cDNA, wherein SEQ ID NO:1345 is a clone designated herein as “DNA328027”.

FIG. 1346 shows the amino acid sequence (SEQ ID NO:1346) derived from the coding sequence of SEQ ID NO:1345 shown in FIG. 1345.

FIG. 1347 shows a nucleotide sequence (SEQ ID NO:1347) of a native sequence PRO5734 cDNA, wherein SEQ ID NO:1347 is a clone designated herein as “DNA329313”.

FIG. 1348 shows the amino acid sequence (SEQ ID NO:1348) derived from the coding sequence of SEQ ID NO:1347 shown in FIG. 1347.

FIG. 1349 A-B shows a nucleotide sequence (SEQ ID NO:1349) of a native sequence PRO84902 cDNA, wherein SEQ ID NO:1349 is a clone designated herein as “DNA329314”.

FIG. 1350 shows the amino acid sequence (SEQ ID NO:1350) derived from the coding sequence of SEQ ID NO:1349 shown in FIG. 1349.

FIG. 1351 shows a nucleotide sequence (SEQ ID NO:1351) of a native sequence PRO37766 cDNA, wherein SEQ ID NO:1351 is a clone designated herein as “DNA227303”.

FIG. 1352 shows the amino acid sequence (SEQ ID NO:1352) derived from the coding sequence of SEQ ID NO:1351 shown in FIG. 1351.

FIG. 1353 shows a nucleotide sequence (SEQ ID NO:1353) of a native sequence PRO84903 cDNA, wherein SEQ ID NO:1353 is a clone designated herein as “DNA329315”.

FIG. 1354 shows the amino acid sequence (SEQ ID NO:1354) derived from the coding sequence of SEQ ID NO:1353 shown in FIG. 1353.

FIG. 1355 A-B shows a nucleotide sequence (SEQ ID NO:1355) of a native sequence PRO84904 cDNA, wherein SEQ ID NO:1355 is a clone designated herein as “DNA329316”.

FIG. 1356 shows the amino acid sequence (SEQ ID NO:1356) derived from the coding sequence of SEQ ID NO:1355 shown in FIG. 1355.

FIG. 1357 shows a nucleotide sequence (SEQ ID NO:1357) of a native sequence PRO81157 cDNA, wherein SEQ ID NO:1357 is a clone designated herein as “DNA329317”.

FIG. 1358 shows the amino acid sequence (SEQ ID NO:1358) derived from the coding sequence of SEQ ID NO:1357 shown in FIG. 1357.

FIG. 1359 shows a nucleotide sequence (SEQ ID NO:1359) of a native sequence cDNA, wherein SEQ ID NO:1359 is a clone designated herein as “DNA329318”.

FIG. 1360 shows a nucleotide sequence (SEQ ID NO:1360) of a native sequence PRO1607 cDNA, wherein SEQ ID NO:1360 is a clone designated herein as “DNA329319”.

FIG. 1361 shows the amino acid sequence (SEQ ID NO:1361) derived from the coding sequence of SEQ ID NO:1360 shown in FIG. 1360.

FIG. 1362 shows a nucleotide sequence (SEQ ID NO:1362) of a native sequence PRO84667 cDNA, wherein SEQ ID NO:1362 is a clone designated herein as “DNA328961”.

FIG. 1363 shows the amino acid sequence (SEQ ID NO:1363) derived from the coding sequence of SEQ ID NO:1362 shown in FIG. 1362.

FIG. 1364 shows a nucleotide sequence (SEQ ID NO:1364) of a native sequence PRO84905 cDNA, wherein SEQ ID NO:1364 is a clone designated herein as “DNA329320”.

FIG. 1365 shows the amino acid sequence (SEQ ID NO:1365) derived from the coding sequence of SEQ ID NO:1364 shown in FIG. 1364.

FIG. 1366 shows a nucleotide sequence (SEQ ID NO:1366) of a native sequence PRO84674 cDNA, wherein SEQ ID NO:1366 is a clone designated herein as “DNA328971”.

FIG. 1367 shows the amino acid sequence (SEQ ID NO:1367) derived from the coding sequence of SEQ ID NO:1366 shown in FIG. 1366.

FIG. 1368 shows a nucleotide sequence (SEQ ID NO:1368) of a native sequence PRO84906 cDNA, wherein SEQ ID NO:1368 is a clone designated herein as “DNA329321”.

FIG. 1369 shows the amino acid sequence (SEQ ID NO:1369) derived from the coding sequence of SEQ ID NO:1368 shown in FIG. 1368.

FIG. 1370 shows a nucleotide sequence (SEQ ID NO:1370) of a native sequence PRO80490 cDNA, wherein SEQ ID NO:1370 is a clone designated herein as “DNA329322”.

FIG. 1371 shows the amino acid sequence (SEQ ID NO:1371) derived from the coding sequence of SEQ ID NO:1370 shown in FIG. 1370.

FIG. 1372 shows a nucleotide sequence (SEQ ID NO:1372) of a native sequence PRO47688 cDNA, wherein SEQ ID NO:1372 is a clone designated herein as “DNA328975”.

FIG. 1373 shows the amino acid sequence (SEQ ID NO:1373) derived from the coding sequence of SEQ ID NO:1372 shown in FIG. 1372.

FIG. 1374 shows a nucleotide sequence (SEQ ID NO:1374) of a native sequence PRO84677 cDNA, wherein SEQ ID NO:1374 is a clone designated herein as “DNA328976”.

FIG. 1375 shows the amino acid sequence (SEQ ID NO:1375) derived from the coding sequence of SEQ ID NO:1374 shown in FIG. 1374.

FIG. 1376 shows a nucleotide sequence (SEQ ID NO:1376) of a native sequence PRO703 cDNA, wherein SEQ ID NO:1376 is a clone designated herein as “DNA323915”.

FIG. 1377 shows the amino acid sequence (SEQ ID NO:1377) derived from the coding sequence of SEQ ID NO:1376 shown in FIG. 1376.

FIG. 1378 shows a nucleotide sequence (SEQ ID NO:1378) of a native sequence PRO84907 cDNA, wherein SEQ ID NO:1378 is a clone designated herein as “DNA329323”.

FIG. 1379 shows the amino acid sequence (SEQ ID NO:1379) derived from the coding sequence of SEQ ID NO:1378 shown in FIG. 1378.

FIG. 1380 A-B shows a nucleotide sequence (SEQ ID NO:1380) of a native sequence PRO1314 cDNA, wherein SEQ ID NO:1380 is a clone designated herein as “DNA324364”.

FIG. 1381 shows the amino acid sequence (SEQ ID NO:1381) derived from the coding sequence of SEQ ID NO:1380 shown in FIG. 1380.

FIG. 1382 shows a nucleotide sequence (SEQ ID NO:1382) of a native sequence PRO84908 cDNA, wherein SEQ ID NO:1382 is a clone designated herein as “DNA329324”.

FIG. 1383 shows the amino acid sequence (SEQ ID NO:1383) derived from the coding sequence of SEQ ID NO:1382 shown in FIG. 1382.

FIG. 1384 shows a nucleotide sequence (SEQ ID NO:1384) of a native sequence PRO69560 cDNA, wherein SEQ ID NO:1384 is a clone designated herein as “DNA287290”.

FIG. 1385 shows the amino acid sequence (SEQ ID NO:1385) derived from the coding sequence of SEQ ID NO:1384 shown in FIG. 1384.

FIG. 1386 shows a nucleotide sequence (SEQ ID NO:1386) of a native sequence PRO84909 cDNA, wherein SEQ ID NO:1386 is a clone designated herein as “DNA329325”.

FIG. 1387 shows the amino acid sequence (SEQ ID NO:1387) derived from the coding sequence of SEQ ID NO:1386 shown in FIG. 1386.

FIG. 1388 shows a nucleotide sequence (SEQ ID NO:1388) of a native sequence PRO37686 cDNA, wherein SEQ ID NO:1388 is a clone designated herein as “DNA227223”.

FIG. 1389 shows the amino acid sequence (SEQ ID NO:1389) derived from the coding sequence of SEQ ID NO:1388 shown in FIG. 1388.

FIG. 1390 A-B shows a nucleotide sequence (SEQ ID NO:1390) of a native sequence PRO84910 cDNA, wherein SEQ ID NO:1390 is a clone designated herein as “DNA329326”.

FIG. 1391 shows the amino acid sequence (SEQ ID NO:1391) derived from the coding sequence of SEQ ID NO:1390 shown in FIG. 1390.

FIG. 1392 shows a nucleotide sequence (SEQ ID NO:1392) of a native sequence PRO83868 cDNA, wherein SEQ ID NO:1392 is a clone designated herein as “DNA327938”.

FIG. 1393 shows the amino acid sequence (SEQ ID NO:1393) derived from the coding sequence of SEQ ID NO:1392 shown in FIG. 1392.

FIG. 1394 shows a nucleotide sequence (SEQ ID NO:1394) of a native sequence PRO83869 cDNA, wherein SEQ ID NO:1394 is a clone designated herein as “DNA327939”.

FIG. 1395 shows the amino acid sequence (SEQ ID NO:1395) derived from the coding sequence of SEQ ID NO:1394 shown in FIG. 1394.

FIG. 1396 shows a nucleotide sequence (SEQ ID NO:1396) of a native sequence PRO84911 cDNA, wherein SEQ ID NO:1396 is a clone designated herein as “DNA329327”.

FIG. 1397 shows the amino acid sequence (SEQ ID NO:1397) derived from the coding sequence of SEQ ID NO:1396 shown in FIG. 1396.

FIG. 1398 A-B shows a nucleotide sequence (SEQ ID NO:1398) of a native sequence PRO51526 cDNA, wherein SEQ ID NO:1398 is a clone designated herein as “DNA26489”.

FIG. 1399 shows the amino acid sequence (SEQ ID NO:1399) derived from the coding sequence of SEQ ID NO:1398 shown in FIG. 1398.

FIG. 1400 shows a nucleotide sequence (SEQ ID NO:1400) of a native sequence PRO4984 cDNA, wherein SEQ ID NO:1400 is a clone designated herein as “DNA304460”.

FIG. 1401 shows the amino acid sequence (SEQ ID NO:1401) derived from the coding sequence of SEQ ID NO:1400 shown in FIG. 1400.

FIG. 1402 shows a nucleotide sequence (SEQ ID NO:1402) of a native sequence PRO84912 cDNA, wherein SEQ ID NO:1402 is a clone designated herein as “DNA329328”.

FIG. 1403 shows the amino acid sequence (SEQ ID NO:1403) derived from the coding sequence of SEQ ID NO:1402 shown in FIG. 1402.

FIG. 1404 shows a nucleotide sequence (SEQ ID NO:1404) of a native sequence PRO793 cDNA, wherein SEQ ID NO:1404 is a clone designated herein as “DNA304495”.

FIG. 1405 shows the amino acid sequence (SEQ ID NO:1405) derived from the coding sequence of SEQ ID NO:1404 shown in FIG. 1404.

FIG. 1406 shows a nucleotide sequence (SEQ ID NO:1406) of a native sequence PRO84913 cDNA, wherein SEQ ID NO:1406 is a clone designated herein as “DNA329329”.

FIG. 1407 shows the amino acid sequence (SEQ ID NO:1407) derived from the coding sequence of SEQ ID NO:1406 shown in FIG. 1406.

FIG. 1408 shows a nucleotide sequence (SEQ ID NO:1408) of a native sequence PRO84914 cDNA, wherein SEQ ID NO:1408 is a clone designated herein as “DNA329330”.

FIG. 1409 shows the amino acid sequence (SEQ ID NO:1409) derived from the coding sequence of SEQ ID NO:1408 shown in FIG. 1408.

FIG. 1410 shows a nucleotide sequence (SEQ ID NO:1410) of a native sequence PRO865 cDNA, wherein SEQ ID NO:1410 is a clone designated herein as “DNA327943”.

FIG. 1411 shows the amino acid sequence (SEQ ID NO:1411) derived from the coding sequence of SEQ ID NO:1410 shown in FIG. 1410.

FIG. 1412 shows a nucleotide sequence (SEQ ID NO:1412) of a native sequence PRO84915 cDNA, wherein SEQ ID NO:1412 is a clone designated herein as “DNA329331”.

FIG. 1413 shows the amino acid sequence (SEQ ID NO:1413) derived from the coding sequence of SEQ ID NO:1412 shown in FIG. 1412.

FIG. 1414 shows a nucleotide sequence (SEQ ID NO:1414) of a native sequence PRO80547 cDNA, wherein SEQ ID NO:1414 is a clone designated herein as “DNA323797”.

FIG. 1415 shows the amino acid sequence (SEQ ID NO:1415) derived from the coding sequence of SEQ ID NO:1414 shown in FIG. 1414.

FIG. 1416 shows a nucleotide sequence (SEQ ID NO:1416) of a native sequence PRO60967 cDNA, wherein SEQ ID NO:1416 is a clone designated herein as “DNA272874”.

FIG. 1417 shows the amino acid sequence (SEQ ID NO:1417) derived from the coding sequence of SEQ ID NO:1416 shown in FIG. 1416.

FIG. 1418 shows a nucleotide sequence (SEQ ID NO:1418) of a native sequence PRO84916 cDNA, wherein SEQ ID NO:1418 is a clone designated herein as “DNA329332”.

FIG. 1419 shows the amino acid sequence (SEQ ID NO:1419) derived from the coding sequence of SEQ ID NO:1418 shown in FIG. 1418.

FIG. 1420 shows a nucleotide sequence (SEQ ID NO:1420) of a native sequence PRO84917 cDNA, wherein SEQ ID NO:1420 is a clone designated herein as “DNA329333”.

FIG. 1421 shows the amino acid sequence (SEQ ID NO:1421) derived from the coding sequence of SEQ ID NO:1420 shown in FIG. 1420.

FIG. 1422 shows a nucleotide sequence (SEQ ID NO:1422) of a native sequence PRO84918 cDNA, wherein SEQ ID NO:1422 is a clone designated herein as “DNA329334”.

FIG. 1423 shows the amino acid sequence (SEQ ID NO:1423) derived from the coding sequence of SEQ ID NO:1422 shown in FIG. 1422.

FIG. 1424 shows a nucleotide sequence (SEQ ID NO:1424) of a native sequence PRO84919 cDNA, wherein SEQ ID NO:1424 is a clone designated herein as “DNA29335”.

FIG. 1425 shows the amino acid sequence (SEQ ID NO:1425) derived from the coding sequence of SEQ ID NO:1424 shown in FIG. 1424.

FIG. 1426 shows a nucleotide sequence (SEQ ID NO:1426) of a native sequence PRO84920 cDNA, wherein SEQ ID NO:1426 is a clone designated herein as “DNA329336”.

FIG. 1427 shows the amino acid sequence (SEQ ID NO:1427) derived from the coding sequence of SEQ ID NO:1426 shown in FIG. 1426.

FIG. 1428 shows a nucleotide sequence (SEQ ID NO:1428) of a native sequence PRO10096 cDNA, wherein SEQ ID NO:1428 is a clone designated herein as “DNA329337”.

FIG. 1429 shows the amino acid sequence (SEQ ID NO:1429) derived from the coding sequence of SEQ ID NO:1428 shown in FIG. 1428.

FIG. 1430 shows a nucleotide sequence (SEQ ID NO:1430) of a native sequence PRO84921 cDNA, wherein SEQ ID NO:1430 is a clone designated herein as “DNA29338”.

FIG. 1431 shows the amino acid sequence (SEQ ID NO:1431) derived from the coding sequence of SEQ ID NO:1430 shown in FIG. 1430.

FIG. 1432 A-B shows a nucleotide sequence (SEQ ID NO:1432) of a native sequence PRO84922 cDNA, wherein SEQ ID NO:1432 is a clone designated herein as “DNA329339”.

FIG. 1433 shows the amino acid sequence (SEQ ID NO:1433) derived from the coding sequence of SEQ ID NO:1432 shown in FIG. 1432.

FIG. 1434 shows a nucleotide sequence (SEQ ID NO:1434) of a native sequence PRO81805 cDNA, wherein SEQ ID NO:1434 is a clone designated herein as “DNA329340”.

FIG. 1435 shows the amino acid sequence (SEQ ID NO:1435) derived from the coding sequence of SEQ ID NO:1434 shown in FIG. 1434.

FIG. 1436 A-B shows a nucleotide sequence (SEQ ID NO:1436) of a native sequence PRO10607 cDNA, wherein SEQ ID NO:1436 is a clone designated herein as “DNA287236”.

FIG. 1437 shows the amino acid sequence (SEQ ID NO:1437) derived from the coding sequence of SEQ ID NO:1436 shown in FIG. 1436.

FIG. 1438 shows a nucleotide sequence (SEQ ID NO:1438) of a native sequence PRO84923 cDNA, wherein SEQ ID NO:1438 is a clone designated herein as “DNA329341”.

FIG. 1439 shows the amino acid sequence (SEQ ID NO:1439) derived from the coding sequence of SEQ ID NO:1438 shown in FIG. 1438.

FIG. 1440 shows a nucleotide sequence (SEQ ID NO:1440) of a native sequence PRO83133 cDNA, wherein SEQ ID NO:1440 is a clone designated herein as “DNA326800”.

FIG. 1441 shows the amino acid sequence (SEQ ID NO:1441) derived from the coding sequence of SEQ ID NO:1440 shown in FIG. 1440.

FIG. 1442 A-B shows a nucleotide sequence (SEQ ID NO:1442) of a native sequence PRO84924 cDNA, wherein SEQ ID NO:1442 is a clone designated herein as “DNA329342”.

FIG. 1443 shows the amino acid sequence (SEQ ID NO:1443) derived from the coding sequence of SEQ ID NO:1442 shown in FIG. 1442.

FIG. 1444 A-B shows a nucleotide sequence (SEQ ID NO:1444) of a native sequence PRO84925 cDNA, wherein SEQ ID NO:1444 is a clone designated herein as “DNA329343”.

FIG. 1445 shows the amino acid sequence (SEQ ID NO:1445) derived from the coding sequence of SEQ ID NO:1444 shown in FIG. 1444.

FIG. 1446 shows a nucleotide sequence (SEQ ID NO:1446) of a native sequence PRO84926 cDNA, wherein SEQ ID NO:1446 is a clone designated herein as “DNA329344”.

FIG. 1447 shows the amino acid sequence (SEQ ID NO:1447) derived from the coding sequence of SEQ ID NO:1446 shown in FIG. 1446.

FIG. 1448 shows a nucleotide sequence (SEQ ID NO:1448) of a native sequence PRO69532 cDNA, wherein SEQ ID NO:1448 is a clone designated herein as “DNA287260”.

FIG. 1449 shows the amino acid sequence (SEQ ID NO:1449) derived from the coding sequence of SEQ ID NO:1448 shown in FIG. 1448.

FIG. 1450 shows a nucleotide sequence (SEQ ID NO:1450) of a native sequence PRO37675 cDNA, wherein SEQ ID NO:1450 is a clone designated herein as “DNA324198”.

FIG. 1451 shows the amino acid sequence (SEQ ID NO:1451) derived from the coding sequence of SEQ ID NO:1450 shown in FIG. 1450.

FIG. 1452 shows a nucleotide sequence (SEQ ID NO:1452) of a native sequence PRO25349 cDNA, wherein SEQ ID NO:1452 is a clone designated herein as “DNA189412”.

FIG. 1453 shows the amino acid sequence (SEQ ID NO:1453) derived from the coding sequence of SEQ ID NO:1452 shown in FIG. 1452.

FIG. 1454 shows a nucleotide sequence (SEQ ID NO:1454) of a native sequence PRO84927 cDNA, wherein SEQ ID NO:1454 is a clone designated herein as “DNA329345”.

FIG. 1455 shows the amino acid sequence (SEQ ID NO:1455) derived from the coding sequence of SEQ ID NO:1454 shown in FIG. 1454.

FIG. 1456 shows a nucleotide sequence (SEQ ID NO:1456) of a native sequence PRO12672 cDNA, wherein SEQ ID NO:1456 is a clone designated herein as “DNA151428”.

FIG. 1457 shows the amino acid sequence (SEQ ID NO:1457) derived from the coding sequence of SEQ ID NO:1456 shown in FIG. 1456.

FIG. 1458 shows a nucleotide sequence (SEQ ID NO:1458) of a native sequence PRO84928 cDNA, wherein SEQ ID NO:1458 is a clone designated herein as “DNA329346”.

FIG. 1459 shows the amino acid sequence (SEQ ID NO:1459) derived from the coding sequence of SEQ ID NO:1458 shown in FIG. 1458.

FIG. 1460 shows a nucleotide sequence (SEQ ID NO:1460) of a native sequence PRO84929 cDNA, wherein SEQ ID NO:1460 is a clone designated herein as “DNA329347”.

FIG. 1461 shows the amino acid sequence (SEQ ID NO:1461) derived from the coding sequence of SEQ ID NO:1460 shown in FIG. 1460.

FIG. 1462 shows a nucleotide sequence (SEQ ID NO:1462) of a native sequence PRO80902 cDNA, wherein SEQ ID NO:1462 is a clone designated herein as “DNA324209”.

FIG. 1463 shows the amino acid sequence (SEQ ID NO:1463) derived from the coding sequence of SEQ ID NO:1462 shown in FIG. 1462.

FIG. 1464 shows a nucleotide sequence (SEQ ID NO:1464) of a native sequence PRO84930 cDNA, wherein SEQ ID NO:1464 is a clone designated herein as “DNA329348”.

FIG. 1465 shows the amino acid sequence (SEQ ID NO:1465) derived from the coding sequence of SEQ ID NO:1464 shown in FIG. 1464.

FIG. 1466 shows a nucleotide sequence (SEQ ID NO:1466) of a native sequence PRO84931 cDNA, wherein SEQ ID NO:1466 is a clone designated herein as “DNA329349”.

FIG. 1467 shows the amino acid sequence (SEQ ID NO:1467) derived from the coding sequence of SEQ ID NO:1466 shown in FIG. 1466.

FIG. 1468 shows a nucleotide sequence (SEQ ID NO:1468) of a native sequence PRO83852 cDNA, wherein SEQ ID NO:1468 is a clone designated herein as “DNA327917”.

FIG. 1469 shows the amino acid sequence (SEQ ID NO:1469) derived from the coding sequence of SEQ ID NO:1468 shown in FIG. 1468.

FIG. 1470 shows a nucleotide sequence (SEQ ID NO:1470) of a native sequence PRO11732 cDNA, wherein SEQ ID NO:1470 is a clone designated herein as “DNA329350”.

FIG. 1471 shows the amino acid sequence (SEQ ID NO:1471) derived from the coding sequence of SEQ ID NO:1470 shown in FIG. 1470.

FIG. 1472 shows a nucleotide sequence (SEQ ID NO:1472) of a native sequence PRO82060 cDNA, wherein SEQ ID NO:1472 is a clone designated herein as “DNA325557”.

FIG. 1473 shows the amino acid sequence (SEQ ID NO:1473) derived from the coding sequence of SEQ ID NO:1472 shown in FIG. 1472.

FIG. 1474 shows a nucleotide sequence (SEQ ID NO:1474) of a native sequence PRO81147 cDNA, wherein SEQ ID NO:1474 is a clone designated herein as “DNA329351”.

FIG. 1475 shows the amino acid sequence (SEQ ID NO:1475) derived from the coding sequence of SEQ ID NO:1474 shown in FIG. 1474.

FIG. 1476 shows a nucleotide sequence (SEQ ID NO:1476) of a native sequence PRO84932 cDNA, wherein SEQ ID NO:1476 is a clone designated herein as “DNA329352”.

FIG. 1477 shows the amino acid sequence (SEQ ID NO:1477) derived from the coding sequence of SEQ ID NO:1476 shown in FIG. 1476.

FIG. 1478 shows a nucleotide sequence (SEQ ID NO:1478) of a native sequence PRO84933 cDNA, wherein SEQ ID NO:1478 is a clone designated herein as “DNA329353”.

FIG. 1479 shows the amino acid sequence (SEQ ID NO:1479) derived from the coding sequence of SEQ ID NO:1478 shown in FIG. 1478.

FIG. 1480 shows a nucleotide sequence (SEQ ID NO:1480) of a native sequence PRO83878 cDNA, wherein SEQ ID NO:1480 is a clone designated herein as “DNA327953”.

FIG. 1481 shows the amino acid sequence (SEQ ID NO:1481) derived from the coding sequence of SEQ ID NO:1480 shown in FIG. 1480.

FIG. 1482 shows a nucleotide sequence (SEQ ID NO:1482) of a native sequence PRO80542 cDNA, wherein SEQ ID NO:1482 is a clone designated herein as “DNA323792”.

FIG. 1483 shows the amino acid sequence (SEQ ID NO:1483) derived from the coding sequence of SEQ ID NO:1482 shown in FIG. 1482.

FIG. 1484 shows a nucleotide sequence (SEQ ID NO:1484) of a native sequence PRO81822 cDNA, wherein SEQ ID NO:1484 is a clone designated herein as “DNA325272”.

FIG. 1485 shows the amino acid sequence (SEQ ID NO:1485) derived from the coding sequence of SEQ ID NO:1484 shown in FIG. 1484.

FIG. 1486 shows a nucleotide sequence (SEQ ID NO:1486) of a native sequence PRO71043 cDNA, wherein SEQ ID NO:1486 is a clone designated herein as “DNA304467”.

FIG. 1487 shows the amino acid sequence (SEQ ID NO:1487) derived from the coding sequence of SEQ ID NO:1486 shown in FIG. 1486.

FIG. 1488A-B shows a nucleotide sequence (SEQ ID NO:1488) of a native sequence PRO52040 cDNA, wherein SEQ ID NO:1488 is a clone designated herein as “DNA257461”.

FIG. 1489 shows the amino acid sequence (SEQ ID NO:1489) derived from the coding sequence of SEQ ID NO:1488 shown in FIG. 1488.

FIG. 1490 shows a nucleotide sequence (SEQ ID NO:1490) of a native sequence PRO84934 cDNA, wherein SEQ ID NO:1490 is a clone designated herein as “DNA329354”.

FIG. 1491 shows the amino acid sequence (SEQ ID NO:1491) derived from the coding sequence of SEQ ID NO:1490 shown in FIG. 1490.

FIG. 1492 shows a nucleotide sequence (SEQ ID NO:1492) of a native sequence PRO83881 cDNA, wherein SEQ ID NO:1492 is a clone designated herein as “DNA327956”.

FIG. 1493 shows the amino acid sequence (SEQ ID NO:1493) derived from the coding sequence of SEQ ID NO:1492 shown in FIG. 1492.

FIG. 1494 shows a nucleotide sequence (SEQ ID NO:1494) of a native sequence PRO51950 cDNA, wherein SEQ ID NO:1494 is a clone designated herein as “DNA257363”.

FIG. 1495 shows the amino acid sequence (SEQ ID NO:1495) derived from the coding sequence of SEQ ID NO:1495 shown in Figure

FIG. 1496 shows a nucleotide sequence (SEQ ID NO:1496) of a native sequence PRO50434 cDNA, wherein SEQ ID NO:1496 is a clone designated herein as “DNA329355”.

FIG. 1497 shows the amino acid sequence (SEQ ID NO:1497) derived from the coding sequence of SEQ ID NO:1496 shown in FIG. 1496.

FIG. 1498 shows a nucleotide sequence (SEQ ID NO:1498) of a native sequence PRO84935 cDNA, wherein SEQ ID NO:1498 is a clone designated herein as “DNA329356”.

FIG. 1499 shows the amino acid sequence (SEQ ID NO:1499) derived from the coding sequence of SEQ ID NO:1498 shown in FIG. 1498.

FIG. 1500 shows a nucleotide sequence (SEQ ID NO:1500) of a native sequence PRO84936 cDNA, wherein SEQ ID NO:1500 is a clone designated herein as “DNA329357”.

FIG. 1501 shows the amino acid sequence (SEQ ID NO:1501) derived from the coding sequence of SEQ ID NO:1500 shown in FIG. 1500.

FIG. 1502 shows a nucleotide sequence (SEQ ID NO:1502) of a native sequence PRO84937 cDNA, wherein SEQ ID NO:1502 is a clone designated herein as “DNA329358”.

FIG. 1503 shows the amino acid sequence (SEQ ID NO:1503) derived from the coding sequence of SEQ ID NO:1502 shown in FIG. 1502.

FIG. 1504 shows a nucleotide sequence (SEQ ID NO:1504) of a native sequence PRO84938 cDNA, wherein SEQ ID NO:1504 is a clone designated herein as “DNA329359”.

FIG. 1505 shows the amino acid sequence (SEQ ID NO:1505) derived from the coding sequence of SEQ ID NO:1504 shown in FIG. 1504.

FIG. 1506 shows a nucleotide sequence (SEQ ID NO:1506) of a native sequence PRO84939 cDNA, wherein SEQ ID NO:1506 is a clone designated herein as “DNA329360”.

FIG. 1507 shows the amino acid sequence (SEQ ID NO:1507) derived from the coding sequence of SEQ ID NO:1506 shown in FIG. 1506.

FIG. 1508 shows a nucleotide sequence (SEQ ID NO:1508) of a native sequence PRO84940 cDNA, wherein SEQ ID NO:1508 is a clone designated herein as “DNA329361”.

FIG. 1509 shows the amino acid sequence (SEQ ID NO:1509) derived from the coding sequence of SEQ ID NO:1508 shown in FIG. 1508.

FIG. 1510 shows a nucleotide sequence (SEQ ID NO:1510) of a native sequence PRO80856 cDNA, wherein SEQ ID NO:1510 is a clone designated herein as “DNA324156”.

FIG. 1511 shows the amino acid sequence (SEQ ID NO:1511) derived from the coding sequence of SEQ ID NO:1510 shown in FIG. 1510.

FIG. 1512 shows a nucleotide sequence (SEQ ID NO:1512) of a native sequence PRO84941 cDNA, wherein SEQ ID NO:1512 is a clone designated herein as “DNA329362”.

FIG. 1513 shows the amino acid sequence (SEQ ID NO:1513) derived from the coding sequence of SEQ ID NO:1512 shown in FIG. 1512.

FIG. 1514 shows a nucleotide sequence (SEQ ID NO:1514) of a native sequence PRO84942 cDNA, wherein SEQ ID NO:1514 is a clone designated herein as “DNA329363”.

FIG. 1515 shows the amino acid sequence (SEQ ID NO:1515) derived from the coding sequence of SEQ ID NO:1514 shown in FIG. 1514.

FIG. 1516 shows a nucleotide sequence (SEQ ID NO:1516) of a native sequence PRO84943 cDNA, wherein SEQ ID NO:1516 is a clone designated herein as “DNA329364”.

FIG. 1517 shows the amino acid sequence (SEQ ID NO:1517) derived from the coding sequence of SEQ ID NO:1516 shown in FIG. 1516.

FIG. 1518 shows a nucleotide sequence (SEQ ID NO:1518) of a native sequence PRO84944 cDNA, wherein SEQ ID NO:1518 is a clone designated herein as “DNA329365”.

FIG. 1519 shows the amino acid sequence (SEQ ID NO:1519) derived from the coding sequence of SEQ ID NO:1518 shown in FIG. 1518.

FIG. 1520 shows a nucleotide sequence (SEQ ID NO:1520) of a native sequence PRO84945 cDNA, wherein SEQ ID NO:1520 is a clone designated herein as “DNA329366”.

FIG. 1521 shows the amino acid sequence (SEQ ID NO:1521) derived from the coding sequence of SEQ ID NO:1520 shown in FIG. 1520.

FIG. 1522 shows a nucleotide sequence (SEQ ID NO:1522) of a native sequence PRO61417 cDNA, wherein SEQ ID NO:1522 is a clone designated herein as “DNA273418”.

FIG. 1523 shows the amino acid sequence (SEQ ID NO:1523) derived from the coding sequence of SEQ ID NO:1522 shown in FIG. 1522.

FIG. 1524 shows a nucleotide sequence (SEQ ID NO:1524) of a native sequence PRO81368 cDNA, wherein SEQ ID NO:1524 is a clone designated herein as “DNA324743”.

FIG. 1525 shows the amino acid sequence (SEQ ID NO:1525) derived from the coding sequence of SEQ ID NO:1524 shown in FIG. 1424.

FIG. 1526 shows a nucleotide sequence (SEQ ID NO:1526) of a native sequence PRO84946 cDNA, wherein SEQ ID NO:1526 is a clone designated herein as “DNA329367”.

FIG. 1527 shows the amino acid sequence (SEQ ID NO:1527) derived from the coding sequence of SEQ ID NO:1526 shown in FIG. 1526.

FIG. 1528 shows a nucleotide sequence (SEQ ID NO:1528) of a native sequence PRO26224 cDNA, wherein SEQ ID NO:1528 is a clone designated herein as “DNA188735”.

FIG. 1529 shows the amino acid sequence (SEQ ID NO:1529) derived from the coding sequence of SEQ ID NO:1528 shown in FIG. 1528.

FIG. 1530 shows a nucleotide sequence (SEQ ID NO:1530) of a native sequence PRO69527 cDNA, wherein SEQ ID NO:1530 is a clone designated herein as “DNA287253”.

FIG. 1531 shows the amino acid sequence (SEQ ID NO:1531) derived from the coding sequence of SEQ ID NO:1530 shown in FIG. 1530.

FIG. 1532 shows a nucleotide sequence (SEQ ID NO:1532) of a native sequence PRO1065 cDNA, wherein SEQ ID NO:1532 is a clone designated herein as “DNA327200”.

FIG. 1533 shows the amino acid sequence (SEQ ID NO:1533) derived from the coding sequence of SEQ ID NO:1532 shown in FIG. 1532.

FIG. 1534 shows a nucleotide sequence (SEQ ID NO:1534) of a native sequence PRO34454 cDNA, wherein SEQ ID NO:1534 is a clone designated herein as “DNA218676”.

FIG. 1535 shows the amino acid sequence (SEQ ID NO:1535) derived from the coding sequence of SEQ ID NO:1534 shown in FIG. 1534.

FIG. 1536 shows a nucleotide sequence (SEQ ID NO:1536) of a native sequence PRO84947 cDNA, wherein SEQ ID NO:1536 is a clone designated herein as “DNA329368”.

FIG. 1537 shows the amino acid sequence (SEQ ID NO:1537) derived from the coding sequence of SEQ ID NO:1536 shown in FIG. 1536.

FIG. 1538 shows a nucleotide sequence (SEQ ID NO:1538) of a native sequence PRO84948 cDNA, wherein SEQ ID NO:1538 is a clone designated herein as “DNA329369”.

FIG. 1539 shows the amino acid sequence (SEQ ID NO:1539) derived from the coding sequence of SEQ ID NO:1538 shown in FIG. 1538.

FIG. 1540 shows a nucleotide sequence (SEQ ID NO:1540) of a native sequence PRO81339 cDNA, wherein SEQ ID NO:1540 is a clone designated herein as “DNA324707”.

FIG. 1541 shows the amino acid sequence (SEQ ID NO:1541) derived from the coding sequence of SEQ ID NO:1540 shown in FIG. 1540.

FIG. 1542 shows a nucleotide sequence (SEQ ID NO:1542) of a native sequence PRO84949 cDNA, wherein SEQ ID NO:1542 is a clone designated herein as “DNA329370”.

FIG. 1543 shows the amino acid sequence (SEQ ID NO:1543) derived from the coding sequence of SEQ ID NO:1542 shown in FIG. 1542.

FIG. 1544 shows a nucleotide sequence (SEQ ID NO:1544) of a native sequence PRO84950 cDNA, wherein SEQ ID NO:1544 is a clone designated herein as “DNA329371”.

FIG. 1545 shows the amino acid sequence (SEQ ID NO:1545) derived from the coding sequence of SEQ ID NO:1544 shown in FIG. 1544.

FIG. 1546 shows a nucleotide sequence (SEQ ID NO:1546) of a native sequence PRO84951 cDNA, wherein SEQ ID NO:1546 is a clone designated herein as “DNA329372”.

FIG. 1547 shows the amino acid sequence (SEQ ID NO:1547) derived from the coding sequence of SEQ ID NO:1546 shown in FIG. 1546.

FIG. 1548 shows a nucleotide sequence (SEQ ID NO:1548) of a native sequence PRO84952 cDNA, wherein SEQ ID NO:1548 is a clone designated herein as “DNA329373”.

FIG. 1549 shows the amino acid sequence (SEQ ID NO:1549) derived from the coding sequence of SEQ ID NO:1548 shown in FIG. 1548.

FIG. 1550 shows a nucleotide sequence (SEQ ID NO:1550) of a native sequence PRO69574 cDNA, wherein SEQ ID NO:1550 is a clone designated herein as “DNA327976”.

FIG. 1551 shows the amino acid sequence (SEQ ID NO:1551) derived from the coding sequence of SEQ ID NO:1550 shown in FIG. 1550.

FIG. 1552 shows a nucleotide sequence (SEQ ID NO:1552) of a native sequence PRO84953 cDNA, wherein SEQ ID NO:1552 is a clone designated herein as “DNA329374”.

FIG. 1553 shows the amino acid sequence (SEQ ID NO:1553) derived from the coding sequence of SEQ ID NO:1552 shown in FIG. 1552.

FIG. 1554 shows a nucleotide sequence (SEQ ID NO:1554) of a native sequence PRO51916 cDNA, wherein SEQ ID NO:1554 is a clone designated herein as “DNA329375”.

FIG. 1555 shows the amino acid sequence (SEQ ID NO:1555) derived from the coding sequence of SEQ ID NO:1554 shown in FIG. 1554.

FIG. 1556 shows a nucleotide sequence (SEQ ID NO:1556) of a native sequence PRO84954 cDNA, wherein SEQ ID NO:1556 is a clone designated herein as “DNA329376”.

FIG. 1557 shows the amino acid sequence (SEQ ID NO:1557) derived from the coding sequence of SEQ ID NO:1556 shown in FIG. 1556.

FIG. 1558 A-B shows a nucleotide sequence (SEQ ID NO:1558) of a native sequence PRO83901 cDNA, wherein SEQ ID NO:1558 is a clone designated herein as “DNA327981”.

FIG. 1559 shows the amino acid sequence (SEQ ID NO:1559) derived from the coding sequence of SEQ ID NO:1558 shown in FIG. 1558.

FIG. 1560 shows a nucleotide sequence (SEQ ID NO:1560) of a native sequence PRO84955 cDNA, wherein SEQ ID NO:1560 is a clone designated herein as “DNA329377”.

FIG. 1561 shows the amino acid sequence (SEQ ID NO:1561) derived from the coding sequence of SEQ ID NO:1560 shown in FIG. 1560.

FIG. 1562 shows a nucleotide sequence (SEQ ID NO:1562) of a native sequence PRO84956 cDNA, wherein SEQ ID NO:1562 is a clone designated herein as “DNA329378”.

FIG. 1563 shows the amino acid sequence (SEQ ID NO:1563) derived from the coding sequence of SEQ ID NO:1562 shown in FIG. 1562.

FIG. 1564 shows a nucleotide sequence (SEQ ID NO:1564) of a native sequence PRO80871 cDNA, wherein SEQ ID NO:1564 is a clone designated herein as “DNA324173”.

FIG. 1565 shows the amino acid sequence (SEQ ID NO:1565) derived from the coding sequence of SEQ ID NO:1564 shown in FIG. 1564.

FIG. 1566 shows a nucleotide sequence (SEQ ID NO:1566) of a native sequence PRO69641 cDNA, wherein SEQ ID NO:1566 is a clone designated herein as “DNA287382”.

FIG. 1567 shows the amino acid sequence (SEQ ID NO:1567) derived from the coding sequence of SEQ ID NO:1566 shown in FIG. 1566.

FIG. 1568 shows a nucleotide sequence (SEQ ID NO:1568) of a native sequence PRO51940 cDNA, wherein SEQ ID NO:1568 is a clone designated herein as “DNA257352”.

FIG. 1569 shows the amino acid sequence (SEQ ID NO:1569) derived from the coding sequence of SEQ ID NO:1568 shown in FIG. 1568.

FIG. 1570 A-B shows a nucleotide sequence (SEQ ID NO:1570) of a native sequence PRO69594 cDNA, wherein SEQ ID NO:1570 is a clone designated herein as “DNA287330”.

FIG. 1571 shows the amino acid sequence (SEQ ID NO:1571) derived from the coding sequence of SEQ ID NO:1570 shown in FIG. 1570.

FIG. 1572A-C shows a nucleotide sequence (SEQ ID NO:1572) of a native sequence PRO84957 cDNA, wherein SEQ ID NO:1572 is a clone designated herein as “DNA329379”.

FIG. 1573 shows the amino acid sequence (SEQ ID NO:1573) derived from the coding sequence of SEQ ID NO:1572 shown in FIG. 1572.

FIG. 1574 shows a nucleotide sequence (SEQ ID NO:1574) of a native sequence PRO80743 cDNA, wherein SEQ ID NO:1574 is a clone designated herein as “DNA329380”.

FIG. 1575 shows the amino acid sequence (SEQ ID NO:1575) derived from the coding sequence of SEQ ID NO:1574 shown in FIG. 1574.

FIG. 1576 shows a nucleotide sequence (SEQ ID NO:1576) of a native sequence cDNA, wherein SEQ ID NO:1576 is a clone designated herein as “DNA329381”.

FIG. 1577 shows a nucleotide sequence (SEQ ID NO:1577) of a native sequence PRO52338 cDNA, wherein SEQ ID NO:1577 is a clone designated herein as “DNA257789”.

FIG. 1578 shows the amino acid sequence (SEQ ID NO:1578) derived from the coding sequence of SEQ ID NO:1577 shown in FIG. 1577.

FIG. 1579 shows a nucleotide sequence (SEQ ID NO:1579) of a native sequence cDNA, wherein SEQ ID NO:1579 is a clone designated herein as “DNA329382”.

FIG. 1580 shows a nucleotide sequence (SEQ ID NO:1580) of a native sequence PRO23603 cDNA, wherein SEQ ID NO:1580 is a clone designated herein as “DNA194211”.

FIG. 1581 shows the amino acid sequence (SEQ ID NO:1581) derived from the coding sequence of SEQ ID NO:1580 shown in FIG. 1580.

FIG. 1582 A-B shows a nucleotide sequence (SEQ ID NO:1582) of a native sequence PRO23253 cDNA, wherein SEQ ID NO:1582 is a clone designated herein as “DNA169523”.

FIG. 1583 shows the amino acid sequence (SEQ ID NO:1583) derived from the coding sequence of SEQ ID NO:1582 shown in FIG. 1582.

FIG. 1584 shows a nucleotide sequence (SEQ ID NO:1584) of a native sequence PRO84959 cDNA, wherein SEQ ID NO:1584 is a clone designated herein as “DNA329383”.

FIG. 1585 shows the amino acid sequence (SEQ ID NO:1585) derived from the coding sequence of SEQ ID NO:1584 shown in FIG. 1584.

FIG. 1586 shows a nucleotide sequence (SEQ ID NO:1586) of a native sequence PRO84960 cDNA, wherein SEQ ID NO:1586 is a clone designated herein as “DNA329384”.

FIG. 1587 shows the amino acid sequence (SEQ ID NO:1587) derived from the coding sequence of SEQ ID NO:1586 shown in FIG. 1586.

FIG. 1588 A-B shows a nucleotide sequence (SEQ ID NO:1588) of a native sequence PRO84961 cDNA, wherein SEQ ID NO:1588 is a clone designated herein as “DNA329385”.

FIG. 1589 shows the amino acid sequence (SEQ ID NO:1589) derived from the coding sequence of SEQ ID NO:1588 shown in FIG. 1588.

FIG. 1590 shows a nucleotide sequence (SEQ ID NO:1590) of a native sequence cDNA, wherein SEQ ID NO:1590 is a clone designated herein as “DNA161646”.

FIG. 1591 shows a nucleotide sequence (SEQ ID NO:1591) of a native sequence PRO84962 cDNA, wherein SEQ ID NO:1591 is a clone designated herein as “DNA329386”.

FIG. 1592 shows the amino acid sequence (SEQ ID NO:1592) derived from the coding sequence of SEQ ID NO:1592 shown in Figure

FIG. 1593 shows a nucleotide sequence (SEQ ID NO:1593) of a native sequence PRO84963 cDNA, wherein SEQ ID NO:1593 is a clone designated herein as “DNA329387”.

FIG. 1594 shows the amino acid sequence (SEQ ID NO:1594) derived from the coding sequence of SEQ ID NO:1593 shown in FIG. 1593.

FIG. 1595 shows a nucleotide sequence (SEQ ID NO:1595) of a native sequence PRO84964 cDNA, wherein SEQ ID NO:1595 is a clone designated herein as “DNA329388”.

FIG. 1596 shows the amino acid sequence (SEQ ID NO:1596) derived from the coding sequence of SEQ ID NO:1595 shown in FIG. 1595.

FIG. 1597 shows a nucleotide sequence (SEQ ID NO:1597) of a native sequence PRO84965 cDNA, wherein SEQ ID NO:1597 is a clone designated herein as “DNA329389”.

FIG. 1598 shows the amino acid sequence (SEQ ID NO:1598) derived from the coding sequence of SEQ ID NO:1597 shown in FIG. 1597.

FIG. 1599A-D shows a nucleotide sequence (SEQ ID NO:1599) of a native sequence PRO84966 cDNA, wherein SEQ ID NO:1599 is a clone designated herein as “DNA329390”.

FIG. 1600 shows the amino acid sequence (SEQ ID NO:1600) derived from the coding sequence of SEQ ID NO:1600 shown in Figure

FIG. 1601A-E shows a nucleotide sequence (SEQ ID NO:1601) of a native sequence PRO84967 cDNA, wherein SEQ ID NO:1601 is a clone designated herein as “DNA329391”.

FIG. 1602 shows the amino acid sequence (SEQ ID NO:1602) derived from the coding sequence of SEQ ID NO:1601 shown in FIG. 1601.

FIG. 1603 A-B shows a nucleotide sequence (SEQ ID NO:1603) of a native sequence PRO84968 cDNA, wherein SEQ ID NO:1603 is a clone designated herein as “DNA329392”.

FIG. 1604 shows the amino acid sequence (SEQ ID NO:1604) derived from the coding sequence of SEQ ID NO:1603 shown in FIG. 1603.

FIG. 1605 A-B shows a nucleotide sequence (SEQ ID NO:1605) of a native sequence PRO81138 cDNA, wherein SEQ ID NO:1605 is a clone designated herein as “DNA327993”.

FIG. 1606 shows the amino acid sequence (SEQ ID NO:1606) derived from the coding sequence of SEQ ID NO:1605 shown in FIG. 1605.

FIG. 1607 shows a nucleotide sequence (SEQ ID NO:1607) of a native sequence cDNA, wherein SEQ ID NO:1607 is a clone designated herein as “DNA155396”.

FIG. 1608 shows a nucleotide sequence (SEQ ID NO:1608) of a native sequence PRO84969 cDNA, wherein SEQ ID NO:1608 is a clone designated herein as “DNA329393”.

FIG. 1609 shows the amino acid sequence (SEQ ID NO:1609) derived from the coding sequence of SEQ ID NO:1608 shown in FIG. 1608.

FIG. 1610 shows a nucleotide sequence (SEQ ID NO:1610) of a native sequence PRO83915 cDNA, wherein SEQ ID NO:1610 is a clone designated herein as “DNA327996”.

FIG. 1611 shows the amino acid sequence (SEQ ID NO:1611) derived from the coding sequence of SEQ ID NO:1611 shown in Figure

FIG. 1612 shows a nucleotide sequence (SEQ ID NO:1612) of a native sequence cDNA, wherein SEQ ID NO:1612 is a clone designated herein as “DNA329394”.

FIG. 1613 A-B shows a nucleotide sequence (SEQ ID NO:1613) of a native sequence PRO84971 cDNA, wherein SEQ ID NO:1613 is a clone designated herein as “DNA329395”.

FIG. 1614 shows the amino acid sequence (SEQ ID NO:1614) derived from the coding sequence of SEQ ID NO:1613 shown in FIG. 1613.

FIG. 1615 shows a nucleotide sequence (SEQ ID NO:1615) of a native sequence cDNA, wherein SEQ ID NO:1615 is a clone designated herein as “DNA228047”.

FIG. 1616 shows a nucleotide sequence (SEQ ID NO:1616) of a native sequence PRO84972 cDNA, wherein SEQ ID NO:1616 is a clone designated herein as “DNA329396”.

FIG. 1617 shows the amino acid sequence (SEQ ID NO:1617) derived from the coding sequence of SEQ ID NO:1616 shown in FIG. 1616.

FIG. 1618 shows a nucleotide sequence (SEQ ID NO:1618) of a native sequence PRO84973 cDNA, wherein SEQ ID NO:1618 is a clone designated herein as “DNA329397”.

FIG. 1619 shows the amino acid sequence (SEQ ID NO:1619) derived from the coding sequence of SEQ ID NO:1618 shown in FIG. 1618.

FIG. 1620 A-B shows a nucleotide sequence (SEQ ID NO:1620) of a native sequence PRO4805 cDNA, wherein SEQ ID NO:1620 is a clone designated herein as “DNA329398”.

FIG. 1621 shows the amino acid sequence (SEQ ID NO:1621) derived from the coding sequence of SEQ ID NO:1620 shown in FIG. 1620.

FIG. 1622 shows a nucleotide sequence (SEQ ID NO:1622) of a native sequence PRO84974 cDNA, wherein SEQ ID NO:1622 is a clone designated herein as “DNA329399”.

FIG. 1623 shows the amino acid sequence (SEQ ID NO:1623) derived from the coding sequence of SEQ ID NO:1622 shown in FIG. 1622.

FIG. 1624 shows a nucleotide sequence (SEQ ID NO:1624) of a native sequence cDNA, wherein SEQ ID NO:1624 is a clone designated herein as “DNA329400”.

FIG. 1625 shows a nucleotide sequence (SEQ ID NO:1625) of a native sequence PRO28544 cDNA, wherein SEQ ID NO:1625 is a clone designated herein as “DNA199090”.

FIG. 1626 shows the amino acid sequence (SEQ ID NO:1626) derived from the coding sequence of SEQ ID NO:1625 shown in FIG. 1625

FIG. 1627 shows a nucleotide sequence (SEQ ID NO:1627) of a native sequence PRO82422 cDNA, wherein SEQ ID NO:1627 is a clone designated herein as “DNA325977”.

FIG. 1628 shows the amino acid sequence (SEQ ID NO:1628) derived from the coding sequence of SEQ ID NO:1627 shown in FIG. 1627.

FIG. 1629 shows a nucleotide sequence (SEQ ID NO:1629) of a native sequence PRO84976 cDNA, wherein SEQ ID NO:1629 is a clone designated herein as “DNA329401”.

FIG. 1630 shows the amino acid sequence (SEQ ID NO:1630) derived from the coding sequence of SEQ ID NO:1629 shown in FIG. 1629.

FIG. 1631 shows a nucleotide sequence (SEQ ID NO:1631) of a native sequence PRO4845 cDNA, wherein SEQ ID NO:1631 is a clone designated herein as “DNA329402”.

FIG. 1632 shows the amino acid sequence (SEQ ID NO:1632) derived from the coding sequence of SEQ ID NO:1631 shown in FIG. 1631.

FIG. 1633 shows a nucleotide sequence (SEQ ID NO:1633) of a native sequence cDNA, wherein SEQ ID NO:1633 is a clone designated herein as “DNA329403”.

FIG. 1634 shows a nucleotide sequence (SEQ ID NO:1634) of a native sequence cDNA, wherein SEQ ID NO:1634 is a clone designated herein as “DNA195699”.

FIG. 1635 shows a nucleotide sequence (SEQ ID NO:1635) of a native sequence PRO71212 cDNA, wherein SEQ ID NO:1635 is a clone designated herein as “DNA304802”.

FIG. 1636 shows the amino acid sequence (SEQ ID NO:1636) derived from the coding sequence of SEQ ID NO:1635 shown in FIG. 1635.

FIG. 1637 shows a nucleotide sequence (SEQ ID NO:1637) of a native sequence cDNA, wherein SEQ ID NO:1637 is a clone designated herein as “DNA328005”.

FIG. 1638 shows a nucleotide sequence (SEQ ID NO:1638) of a native sequence PRO82972 cDNA, wherein SEQ ID NO:1638 is a clone designated herein as “DNA329404”.

FIG. 1639 shows the amino acid sequence (SEQ ID NO:1639) derived from the coding sequence of SEQ ID NO:1638 shown in FIG. 1638.

FIG. 1640 shows a nucleotide sequence (SEQ ID NO:1640) of a native sequence cDNA, wherein SEQ ID NO:1640 is a clone designated herein as “DNA196561”.

FIG. 1641 shows a nucleotide sequence (SEQ ID NO:1641) of a native sequence cDNA, wherein SEQ ID NO:1641 is a clone designated herein as “DNA210184”.

FIG. 1642 shows a nucleotide sequence (SEQ ID NO:1642) of a native sequence cDNA, wherein SEQ ID NO:1642 is a clone designated herein as “DNA329405”.

FIG. 1643 shows a nucleotide sequence (SEQ ID NO:1643) of a native sequence PRO83926 cDNA, wherein SEQ ID NO:1643 is a clone designated herein as “DNA328008”.

FIG. 1644 shows the amino acid sequence (SEQ ID NO:1644) derived from the coding sequence of SEQ ID NO:1643 shown in FIG. 1643.

FIG. 1645 shows a nucleotide sequence (SEQ ID NO:1645) of a native sequence PRO84979 cDNA, wherein SEQ ID NO:1645 is a clone designated herein as “DNA329406”.

FIG. 1646 shows the amino acid sequence (SEQ ID NO:1646) derived from the coding sequence of SEQ ID NO:1645 shown in FIG. 1645.

FIG. 1647 A-B shows a nucleotide sequence (SEQ ID NO:1647) of a native sequence PRO84980 cDNA, wherein SEQ ID NO:1647 is a clone designated herein as “DNA329407”.

FIG. 1648 shows the amino acid sequence (SEQ ID NO:1648) derived from the coding sequence of SEQ ID NO:1647 shown in FIG. 1647.

FIG. 1649 shows a nucleotide sequence (SEQ ID NO:1649) of a native sequence PRO71045 cDNA, wherein SEQ ID NO:1649 is a clone designated herein as “DNA304469”.

FIG. 1650 shows the amino acid sequence (SEQ ID NO:1650) derived from the coding sequence of SEQ ID NO:1649 shown in FIG. 1649.

FIG. 1651 shows a nucleotide sequence (SEQ ID NO:1651) of a native sequence PRO70021 cDNA, wherein SEQ ID NO:1651 is a clone designated herein as “DNA288261”.

FIG. 1652 shows the amino acid sequence (SEQ ID NO:1652) derived from the coding sequence of SEQ ID NO:1651 shown in FIG. 1651.

FIG. 1653 shows a nucleotide sequence (SEQ ID NO:1653) of a native sequence PRO38893 cDNA, wherein SEQ ID NO:1653 is a clone designated herein as “DNA329408”.

FIG. 1654 shows the amino acid sequence (SEQ ID NO:1654) derived from the coding sequence of SEQ ID NO:1653 shown in FIG. 1653.

FIG. 1655 shows a nucleotide sequence (SEQ ID NO:1655) of a native sequence PRO84981 cDNA, wherein SEQ ID NO:1655 is a clone designated herein as “DNA329409”.

FIG. 1656 shows the amino acid sequence (SEQ ID NO:1656) derived from the coding sequence of SEQ ID NO:1655 shown in FIG. 1655.

FIG. 1657 shows a nucleotide sequence (SEQ ID NO:1657) of a native sequence PRO84982 cDNA, wherein SEQ ID NO:1657 is a clone designated herein as “DNA329410”.

FIG. 1658 shows the amino acid sequence (SEQ ID NO:1658) derived from the coding sequence of SEQ ID NO:1657 shown in FIG. 1657.

FIG. 1659 shows a nucleotide sequence (SEQ ID NO:1659) of a native sequence PRO84983 cDNA, wherein SEQ ID NO:1659 is a clone designated herein as “DNA329411”.

FIG. 1660 shows the amino acid sequence (SEQ ID NO:1660) derived from the coding sequence of SEQ ID NO:1659 shown in FIG. 1659.

FIG. 1661 shows a nucleotide sequence (SEQ ID NO:1661) of a native sequence PRO51958 cDNA, wherein SEQ ID NO:1661 is a clone designated herein as “DNA257371”.

FIG. 1662 shows the amino acid sequence (SEQ ID NO:1662) derived from the coding sequence of SEQ ID NO:1661 shown in FIG. 1661.

FIG. 1663 shows a nucleotide sequence (SEQ ID NO:1663) of a native sequence PRO84984 cDNA, wherein SEQ ID NO:1663 is a clone designated herein as “DNA329412”.

FIG. 1664 shows the amino acid sequence (SEQ ID NO:1664) derived from the coding sequence of SEQ ID NO:1663 shown in FIG. 1663.

FIG. 1665 shows a nucleotide sequence (SEQ ID NO:1665) of a native sequence PRO84985 cDNA, wherein SEQ ID NO:1665 is a clone designated herein as “DNA329413”.

FIG. 1666 shows the amino acid sequence (SEQ ID NO:1666) derived from the coding sequence of SEQ ID NO:1665 shown in FIG. 1665.

FIG. 1667 shows a nucleotide sequence (SEQ ID NO:1667) of a native sequence PRO84986 cDNA, wherein SEQ ID NO:1667 is a clone designated herein as “DNA329414”.

FIG. 1668 shows the amino acid sequence (SEQ ID NO:1668) derived from the coding sequence of SEQ ID NO:1667 shown in FIG. 1667.

FIG. 1669 shows a nucleotide sequence (SEQ ID NO:1669) of a native sequence PRO84987 cDNA, wherein SEQ ID NO:1669 is a clone designated herein as “DNA329415”.

FIG. 1670 shows the amino acid sequence (SEQ ID NO:1670) derived from the coding sequence of SEQ ID NO:1669 shown in FIG. 1669.

FIG. 1671 shows a nucleotide sequence (SEQ ID NO:1671) of a native sequence PRO84988 cDNA, wherein SEQ ID NO:1671 is a clone designated herein as “DNA329416”.

FIG. 1672 shows the amino acid sequence (SEQ ID NO:1672) derived from the coding sequence of SEQ ID NO:1671 shown in FIG. 1671.

FIG. 1673 shows a nucleotide sequence (SEQ ID NO:1673) of a native sequence PRO84989 cDNA, wherein SEQ ID NO:1673 is a clone designated herein as “DNA329417”.

FIG. 1674 shows the amino acid sequence (SEQ ID NO:1674) derived from the coding sequence of SEQ ID NO:1673 shown in FIG. 1673.

FIG. 1675 shows a nucleotide sequence (SEQ ID NO:1675) of a native sequence PRO19906 cDNA, wherein SEQ ID NO:1675 is a clone designated herein as “DNA329418”.

FIG. 1676 shows the amino acid sequence (SEQ ID NO:1676) derived from the coding sequence of SEQ ID NO:1675 shown in FIG. 1675.

FIG. 1677 shows a nucleotide sequence (SEQ ID NO:1677) of a native sequence PRO69630 cDNA, wherein SEQ ID NO:1677 is a clone designated herein as “DNA287370”.

FIG. 1678 shows the amino acid sequence (SEQ ID NO:1678) derived from the coding sequence of SEQ ID NO:1677 shown in FIG. 1677.

FIG. 1679 A-B shows a nucleotide sequence (SEQ ID NO:1679) of a native sequence PRO84990 cDNA, wherein SEQ ID NO:1679 is a clone designated herein as “DNA329419”.

FIG. 1680 shows the amino acid sequence (SEQ ID NO:1680) derived from the coding sequence of SEQ ID NO:1679 shown in FIG. 1679.

FIG. 1681 shows a nucleotide sequence (SEQ ID NO:1681) of a native sequence cDNA, wherein SEQ ID NO:1681 is a clone designated herein as “DNA329420”.

FIG. 1682A-B shows a nucleotide sequence (SEQ ID NO:1682) of a native sequence PRO84992 cDNA, wherein SEQ ID NO:1682 is a clone designated herein as “DNA329421”.

FIG. 1683 shows the amino acid sequence (SEQ ID NO:1683) derived from the coding sequence of SEQ ID NO:1682 shown in FIG. 1682.

FIG. 1684 A-B shows a nucleotide sequence (SEQ ID NO:1684) of a native sequence PRO84993 cDNA, wherein SEQ ID NO:1684 is a clone designated herein as “DNA329422”.

FIG. 1685 shows the amino acid sequence (SEQ ID NO:1685) derived from the coding sequence of SEQ ID NO:1684 shown in FIG. 1684.

FIG. 1686 A-B shows a nucleotide sequence (SEQ ID NO:1686) of a native sequence PRO84994 cDNA, wherein SEQ ID NO:1686 is a clone designated herein as “DNA329423”.

FIG. 1687 shows the amino acid sequence (SEQ ID NO:1687) derived from the coding sequence of SEQ ID NO:1686 shown in FIG. 1686.

FIG. 1688 shows a nucleotide sequence (SEQ ID NO:1688) of a native sequence PRO84995 cDNA, wherein SEQ ID NO:1688 is a clone designated herein as “DNA329424”.

FIG. 1689 shows the amino acid sequence (SEQ ID NO:1689) derived from the coding sequence of SEQ ID NO:1688 shown in FIG. 1688.

FIG. 1690 shows a nucleotide sequence (SEQ ID NO:1690) of a native sequence cDNA, wherein SEQ ID NO:1690 is a clone designated herein as “DNA329425”.

FIG. 1691 shows a nucleotide sequence (SEQ ID NO:1691) of a native sequence PRO84997 cDNA, wherein SEQ ID NO:1691 is a clone designated herein as “DNA329426”.

FIG. 1692 shows the amino acid sequence (SEQ ID NO:1692) derived from the coding sequence of SEQ ID NO:1691 shown in FIG. 1691.

FIG. 1693 shows a nucleotide sequence (SEQ ID NO:1693) of a native sequence PRO956 cDNA, wherein SEQ ID NO:1693 is a clone designated herein as “DNA56350”.

FIG. 1694 shows the amino acid sequence (SEQ ID NO:1694) derived from the coding sequence of SEQ ID NO:1693 shown in FIG. 1693.

FIG. 1695 shows a nucleotide sequence (SEQ ID NO:1695) of a native sequence PRO84998 cDNA, wherein SEQ ID NO:1695 is a clone designated herein as “DNA329427”.

FIG. 1696 shows the amino acid sequence (SEQ ID NO:1696) derived from the coding sequence of SEQ ID NO:1695 shown in FIG. 1695.

FIG. 1697 shows a nucleotide sequence (SEQ ID NO:1697) of a native sequence PRO84999 cDNA, wherein SEQ ID NO:1697 is a clone designated herein as “DNA329428”.

FIG. 1698 shows the amino acid sequence (SEQ ID NO:1698) derived from the coding sequence of SEQ ID NO:1697 shown in FIG. 1697.

FIG. 1699 shows a nucleotide sequence (SEQ ID NO:1699) of a native sequence cDNA, wherein SEQ ID NO:1699 is a clone designated herein as “DNA195822”.

FIG. 1700 shows a nucleotide sequence (SEQ ID NO:1700) of a native sequence PRO23314 cDNA, wherein SEQ ID NO:1700 is a clone designated herein as “DNA193896”.

FIG. 1701 shows the amino acid sequence (SEQ ID NO:1701) derived from the coding sequence of SEQ ID NO:1700 shown in FIG. 1700.

FIG. 1702 shows a nucleotide sequence (SEQ ID NO:1702) of a native sequence PRO85000 cDNA, wherein SEQ ID NO:1702 is a clone designated herein as “DNA329429”.

FIG. 1703 shows the amino acid sequence (SEQ ID NO:1703) derived from the coding sequence of SEQ ID NO:1703 shown in Figure

FIG. 1704 shows a nucleotide sequence (SEQ ID NO:1704) of a native sequence PRO83945 cDNA, wherein SEQ ID NO:1704 is a clone designated herein as “DNA328028”.

FIG. 1705 shows the amino acid sequence (SEQ ID NO:1705) derived from the coding sequence of SEQ ID NO:1704 shown in FIG. 1704.

FIG. 1706 shows a nucleotide sequence (SEQ ID NO:1706) of a native sequence PRO38524 cDNA, wherein SEQ ID NO:1706 is a clone designated herein as “DNA329430”.

FIG. 1707 shows the amino acid sequence (SEQ ID NO:1707) derived from the coding sequence of SEQ ID NO:1706 shown in FIG. 1706.

FIG. 1708A-C shows a nucleotide sequence (SEQ ID NO:1708) of a native sequence PRO12637 cDNA, wherein SEQ ID NO:1708 is a clone designated herein as “DNA151207”.

FIG. 1709 shows the amino acid sequence (SEQ ID NO:1709) derived from the coding sequence of SEQ ID NO:1708 shown in FIG. 1708.

FIG. 1710 shows a nucleotide sequence (SEQ ID NO:1710) of a native sequence PRO85001 cDNA, wherein SEQ ID NO:1710 is a clone designated herein as “DNA329431”.

FIG. 1711 shows the amino acid sequence (SEQ ID NO:1711) derived from the coding sequence of SEQ ID NO:1710 shown in FIG. 1710.

FIG. 1712 shows a nucleotide sequence (SEQ ID NO:1712) of a native sequence PRO85002 cDNA, wherein SEQ ID NO:1712 is a clone designated herein as “DNA329432”.

FIG. 1713 shows the amino acid sequence (SEQ ID NO:1713) derived from the coding sequence of SEQ ID NO:1712 shown in FIG. 1712.

FIG. 1714 shows a nucleotide sequence (SEQ ID NO:1714) of a native sequence PRO85003 cDNA, wherein SEQ ID NO:1714 is a clone designated herein as “DNA329433”.

FIG. 1715 shows the amino acid sequence (SEQ ID NO:1715) derived from the coding sequence of SEQ ID NO:1714 shown in FIG. 1714.

FIG. 1716 shows a nucleotide sequence (SEQ ID NO:1716) of a native sequence PRO85004 cDNA, wherein SEQ ID NO:1716 is a clone designated herein as “DNA329434”.

FIG. 1717 shows the amino acid sequence (SEQ ID NO:1717) derived from the coding sequence of SEQ ID NO:1716 shown in FIG. 1716.

FIG. 1718 shows a nucleotide sequence (SEQ ID NO:1718) of a native sequence PRO52418 cDNA, wherein SEQ ID NO:1718 is a clone designated herein as “DNA257884”.

FIG. 1719 shows the amino acid sequence (SEQ ID NO:1719) derived from the coding sequence of SEQ ID NO:1718 shown in FIG. 1718.

FIG. 1720A-C shows a nucleotide sequence (SEQ ID NO:1720) of a native sequence PRO84288 cDNA, wherein SEQ ID NO:1720 is a clone designated herein as “DNA328462”.

FIG. 1721 shows the amino acid sequence (SEQ ID NO:1721) derived from the coding sequence of SEQ ID NO:1720 shown in FIG. 1720

FIG. 1722 shows a nucleotide sequence (SEQ ID NO:1722) of a native sequence PRO85005 cDNA, wherein SEQ ID NO:1722 is a clone designated herein as “DNA329435”.

FIG. 1723 shows the amino acid sequence (SEQ ID NO:1723) derived from the coding sequence of SEQ ID NO:1722 shown in FIG. 1722.

FIG. 1724A-B shows a nucleotide sequence (SEQ ID NO:1724) of a native sequence PRO85006 cDNA, wherein SEQ ID NO:1724 is a clone designated herein as “DNA329436”.

FIG. 1725 shows the amino acid sequence (SEQ ID NO:1725) derived from the coding sequence of SEQ ID NO:1724 shown in FIG. 1724

FIG. 1726 shows a nucleotide sequence (SEQ ID NO:1726) of a native sequence PRO85007 cDNA, wherein SEQ ID NO:1726 is a clone designated herein as “DNA329437”.

FIG. 1727 shows the amino acid sequence (SEQ ID NO:1727) derived from the coding sequence of SEQ ID NO:1726 shown in FIG. 1726.

FIG. 1728A-B shows a nucleotide sequence (SEQ ID NO:1728) of a native sequence PRO85008 cDNA, wherein SEQ ID NO:1728 is a clone designated herein as “DNA329438”.

FIG. 1729 shows the amino acid sequence (SEQ ID NO:1729) derived from the coding sequence of SEQ ID NO:1728 shown in FIG. 1728.

FIG. 1730 shows a nucleotide sequence (SEQ ID NO:1730) of a native sequence cDNA, wherein SEQ ID NO:1730 is a clone designated herein as “DNA329439”.

FIG. 1731 shows a nucleotide sequence (SEQ ID NO:1731) of a native sequence PRO12626 cDNA, wherein SEQ ID NO:1731 is a clone designated herein as “DNA151170”.

FIG. 1732 shows the amino acid sequence (SEQ ID NO:1732) derived from the coding sequence of SEQ ID NO:1731 shown in FIG. 1731.

FIG. 1733 shows a nucleotide sequence (SEQ ID NO:1733) of a native sequence PRO85009 cDNA, wherein SEQ ID NO:1733 is a clone designated herein as “DNA329440”.

FIG. 1734 shows the amino acid sequence (SEQ ID NO:1734) derived from the coding sequence of SEQ ID NO:1733 shown in FIG. 1733

FIG. 1735 shows a nucleotide sequence (SEQ ID NO:1735) of a native sequence PRO83963 cDNA, wherein SEQ ID NO:1735 is a clone designated herein as “DNA328049”.

FIG. 1736 shows the amino acid sequence (SEQ ID NO:1736) derived from the coding sequence of SEQ ID NO:1735 shown in FIG. 1735

FIG. 1737 shows a nucleotide sequence (SEQ ID NO:1737) of a native sequence PRO85010 cDNA, wherein SEQ ID NO:1737 is a clone designated herein as “DNA329441”.

FIG. 1738 shows the amino acid sequence (SEQ ID NO:1738) derived from the coding sequence of SEQ ID NO:1737 shown in FIG. 1737.

FIG. 1739 shows a nucleotide sequence (SEQ ID NO:1739) of a native sequence cDNA, wherein SEQ ID NO:1739 is a clone designated herein as “DNA329442”.

FIG. 1740 shows a nucleotide sequence (SEQ ID NO:1740) of a native sequence PRO85011 cDNA, wherein SEQ ID NO:1740 is a clone designated herein as “DNA329443”.

FIG. 1741 shows the amino acid sequence (SEQ ID NO:1741) derived from the coding sequence of SEQ ID NO:1740 shown in FIG. 1740.

FIG. 1742 shows a nucleotide sequence (SEQ ID NO:1742) of a native sequence PRO85012 cDNA, wherein SEQ ID NO:1742 is a clone designated herein as “DNA329444”.

FIG. 1743 shows the amino acid sequence (SEQ ID NO:1743) derived from the coding sequence of SEQ ID NO:1742 shown in FIG. 1742.

FIG. 1744 shows a nucleotide sequence (SEQ ID NO:1744) of a native sequence PRO85013 cDNA, wherein SEQ ID NO:1744 is a clone designated herein as “DNA329445”.

FIG. 1745 shows the amino acid sequence (SEQ ID NO:1745) derived from the coding sequence of SEQ ID NO:1744 shown in FIG. 1744.

FIG. 1746 shows a nucleotide sequence (SEQ ID NO:1746) of a native sequence PRO85014 cDNA, wherein SEQ ID NO:1746 is a clone designated herein as “DNA329446”.

FIG. 1747 shows the amino acid sequence (SEQ ID NO:1747) derived from the coding sequence of SEQ ID NO:1746 shown in FIG. 1746.

FIG. 1748 shows a nucleotide sequence (SEQ ID NO:1748) of a native sequence PRO61074 cDNA, wherein SEQ ID NO:1748 is a clone designated herein as “DNA273002”.

FIG. 1749 shows the amino acid sequence (SEQ ID NO:1749) derived from the coding sequence of SEQ ID NO:1748 shown in FIG. 1748.

FIG. 1750 shows a nucleotide sequence (SEQ ID NO:1750) of a native sequence PRO85015 cDNA, wherein SEQ ID NO:1750 is a clone designated herein as “DNA329447”.

FIG. 1751 shows the amino acid sequence (SEQ ID NO:1751) derived from the coding sequence of SEQ ID NO:1750 shown in FIG. 1750.

FIG. 1752A-B shows a nucleotide sequence (SEQ ID NO:1752) of a native sequence PRO83968 cDNA, wherein SEQ ID NO:1752 is a clone designated herein as “DNA328054”.

FIG. 1753 shows the amino acid sequence (SEQ ID NO:1753) derived from the coding sequence of SEQ ID NO:1752 shown in FIG. 1752.

FIG. 1754 shows a nucleotide sequence (SEQ ID NO:1754) of a native sequence PRO85016 cDNA, wherein SEQ ID NO:1754 is a clone designated herein as “DNA329448”.

FIG. 1755 shows the amino acid sequence (SEQ ID NO:1755) derived from the coding sequence of SEQ ID NO:1754 shown in FIG. 1754.

FIG. 1756 shows a nucleotide sequence (SEQ ID NO:1756) of a native sequence PRO85017 cDNA, wherein SEQ ID NO:1756 is a clone designated herein as “DNA329449”.

FIG. 1757 shows the amino acid sequence (SEQ ID NO:1757) derived from the coding sequence of SEQ ID NO:1756 shown in FIG. 1756.

FIG. 1758 shows a nucleotide sequence (SEQ ID NO:1758) of a native sequence cDNA, wherein SEQ ID NO:1758 is a clone designated herein as “DNA161163”.

FIG. 1759 shows a nucleotide sequence (SEQ ID NO:1759) of a native sequence PRO80483 cDNA, wherein SEQ ID NO:1759 is a clone designated herein as “DNA323723”.

FIG. 1760 shows the amino acid sequence (SEQ ID NO:1760) derived from the coding sequence of SEQ ID NO:1759 shown in FIG. 1759.

FIG. 1761 shows a nucleotide sequence (SEQ ID NO:1761) of a native sequence PRO85018 cDNA, wherein SEQ ID NO:1761 is a clone designated herein as “DNA329450”.

FIG. 1762 shows the amino acid sequence (SEQ ID NO:1762) derived from the coding sequence of SEQ ID NO:1761 shown in FIG. 1761.

FIG. 1763 shows a nucleotide sequence (SEQ ID NO:1763) of a native sequence PRO85019 cDNA, wherein SEQ ID NO:1763 is a clone designated herein as “DNA329451”.

FIG. 1764 shows the amino acid sequence (SEQ ID NO:1764) derived from the coding sequence of SEQ ID NO:1763 shown in FIG. 1763.

FIG. 1765 shows a nucleotide sequence (SEQ ID NO:1765) of a native sequence PRO85020 cDNA, wherein SEQ ID NO:1765 is a clone designated herein as “DNA329452”.

FIG. 1766 shows the amino acid sequence (SEQ ID NO:1766) derived from the coding sequence of SEQ ID NO:1765 shown in FIG. 1765.

FIG. 1767 shows a nucleotide sequence (SEQ ID NO:1767) of a native sequence cDNA, wherein SEQ ID NO:1767 is a clone designated herein as “DNA329453”.

FIG. 1768 shows a nucleotide sequence (SEQ ID NO:1768) of a native sequence PRO85022 cDNA, wherein SEQ ID NO:1768 is a clone designated herein as “DNA329454”.

FIG. 1769 shows the amino acid sequence (SEQ ID NO:1769) derived from the coding sequence of SEQ ID NO:1768 shown in FIG. 1768.

FIG. 1770 shows a nucleotide sequence (SEQ ID NO:1770) of a native sequence PRO82968 cDNA, wherein SEQ ID NO:1770 is a clone designated herein as “DNA329455”.

FIG. 1771 shows the amino acid sequence (SEQ ID NO:1771) derived from the coding sequence of SEQ ID NO:1771 shown in Figure

FIG. 1772 shows a nucleotide sequence (SEQ ID NO:1772) of a native sequence PRO11849 cDNA, wherein SEQ ID NO:1772 is a clone designated herein as “DNA151503”.

FIG. 1773 shows the amino acid sequence (SEQ ID NO:1773) derived from the coding sequence of SEQ ID NO:1772 shown in FIG. 1772.

FIG. 1774 shows a nucleotide sequence (SEQ ID NO:1774) of a native sequence PRO85023 cDNA, wherein SEQ ID NO:1714 is a clone designated herein as “DNA329456”.

FIG. 1775 shows the amino acid sequence (SEQ ID NO:1775) derived from the coding sequence of SEQ ID NO:1774 shown in FIG. 1774.

FIG. 1776 shows a nucleotide sequence (SEQ ID NO:1776) of a native sequence PRO11901 cDNA, wherein SEQ ID NO:1776 is a clone designated herein as “DNA151580”.

FIG. 1777 shows the amino acid sequence (SEQ ID NO:1777) derived from the coding sequence of SEQ ID NO:1777 shown in Figure

FIG. 1778 shows a nucleotide sequence (SEQ ID NO:1778) of a native sequence PRO85024 cDNA, wherein SEQ ID NO:1778 is a clone designated herein as “DNA329457”.

FIG. 1779 shows the amino acid sequence (SEQ ID NO:1779) derived from the coding sequence of SEQ ID NO:1778 shown in FIG. 1778.

FIG. 1780 shows a nucleotide sequence (SEQ ID NO:1780) of a native sequence PRO12397 cDNA, wherein SEQ ID NO:1780 is a clone designated herein as “DNA150660”.

FIG. 1781 shows the amino acid sequence (SEQ ID NO:1781) derived from the coding sequence of SEQ ID NO:1780 shown in FIG. 1780.

FIG. 1782 shows a nucleotide sequence (SEQ ID NO:1782) of a native sequence PRO85025 cDNA, wherein SEQ ID NO:1782 is a clone designated herein as “DNA329458”.

FIG. 1783 shows the amino acid sequence (SEQ ID NO:1783) derived from the coding sequence of SEQ ID NO:1782 shown in FIG. 1782.

FIG. 1784 shows a nucleotide sequence (SEQ ID NO:1784) of a native sequence PRO85026 cDNA, wherein SEQ ID NO:1784 is a clone designated herein as “DNA329459”.

FIG. 1785 shows the amino acid sequence (SEQ ID NO:1785) derived from the coding sequence of SEQ ID NO:1784 shown in FIG. 1784.

FIG. 1786 shows a nucleotide sequence (SEQ ID NO:1786) of a native sequence PRO85027 cDNA, wherein SEQ ID NO:1786 is a clone designated herein as “DNA329460”.

FIG. 1787 shows the amino acid sequence (SEQ ID NO:1787) derived from the coding sequence of SEQ ID NO:1786 shown in FIG. 1786.

FIG. 1788 shows a nucleotide sequence (SEQ ID NO:1788) of a native sequence PRO85028 cDNA, wherein SEQ ID NO:1788 is a clone designated herein as “DNA329461”.

FIG. 1789 shows the amino acid sequence (SEQ ID NO:1789) derived from the coding sequence of SEQ ID NO:1788 shown in FIG. 1788.

FIG. 1790 shows a nucleotide sequence (SEQ ID NO:1790) of a native sequence cDNA, wherein SEQ ID NO:1790 is a clone designated herein as “DNA153924”.

FIG. 1791 shows a nucleotide sequence (SEQ ID NO:1791) of a native sequence PRO37992 cDNA, wherein SEQ ID NO:1791 is a clone designated herein as “DNA227529”.

FIG. 1792 shows the amino acid sequence (SEQ ID NO:1792) derived from the coding sequence of SEQ ID NO:1791 shown in FIG. 1791.

FIG. 1793 shows a nucleotide sequence (SEQ ID NO:1793) of a native sequence cDNA, wherein SEQ ID NO:1793 is a clone designated herein as “DNA328074”.

FIG. 1794 shows a nucleotide sequence (SEQ ID NO:1794) of a native sequence PRO85029 cDNA, wherein SEQ ID NO:1794 is a clone designated herein as “DNA329462”.

FIG. 1795 shows the amino acid sequence (SEQ ID NO:1795) derived from the coding sequence of SEQ ID NO:1794 shown in FIG. 1794.

FIG. 1796 shows a nucleotide sequence (SEQ ID NO:1796) of a native sequence PRO85030 cDNA, wherein SEQ ID NO:1796 is a clone designated herein as “DNA329463”.

FIG. 1797 shows the amino acid sequence (SEQ ID NO:1797) derived from the coding sequence of SEQ ID NO:1797 shown in Figure

FIG. 1798 shows a nucleotide sequence (SEQ ID NO:1798) of a native sequence PRO83994 cDNA, wherein SEQ ID NO:1798 is a clone designated herein as “DNA328082”.

FIG. 1799 shows the amino acid sequence (SEQ ID NO:1799) derived from the coding sequence of SEQ ID NO:1798 shown in FIG. 1798.

FIG. 1800 shows a nucleotide sequence (SEQ ID NO:1800) of a native sequence cDNA, wherein SEQ ID NO:1800 is a clone designated herein as “DNA260010”.

FIG. 1801 shows a nucleotide sequence (SEQ ID NO:1801) of a native sequence PRO85031 cDNA, wherein SEQ ID NO:1801 is a clone designated herein as “DNA329464”.

FIG. 1802 shows the amino acid sequence (SEQ ID NO:1802) derived from the coding sequence of SEQ ID NO:1801 shown in FIG. 1801.

FIG. 1803 shows a nucleotide sequence (SEQ ID NO:1803) of a native sequence cDNA, wherein SEQ ID NO:1803 is a clone designated herein as “DNA257575”.

FIG. 1804 shows a nucleotide sequence (SEQ ID NO:1804) of a native sequence PRO69678 cDNA, wherein SEQ ID NO:1804 is a clone designated herein as “DNA287421”.

FIG. 1805 shows the amino acid sequence (SEQ ID NO:1805) derived from the coding sequence of SEQ ID NO:1804 shown in FIG. 1804.

FIG. 1806 shows a nucleotide sequence (SEQ ID NO:1806) of a native sequence PRO84001 cDNA, wherein SEQ ID NO:1806 is a clone designated herein as “DNA328090”.

FIG. 1807 shows the amino acid sequence (SEQ ID NO:1807) derived from the coding sequence of SEQ ID NO:1806 shown in FIG. 1806.

FIG. 1808 shows a nucleotide sequence (SEQ ID NO:1808) of a native sequence PRO85032 cDNA, wherein SEQ ID NO:1808 is a clone designated herein as “DNA329465”.

FIG. 1809 shows the amino acid sequence (SEQ ID NO:1809) derived from the coding sequence of SEQ ID NO:1808 shown in FIG. 1808.

FIG. 1810 shows a nucleotide sequence (SEQ ID NO:1810) of a native sequence PRO83478 cDNA, wherein SEQ ID NO:1810 is a clone designated herein as “DNA327205”.

FIG. 1811 shows the amino acid sequence (SEQ ID NO:1811) derived from the coding sequence of SEQ ID NO:1810 shown in FIG. 1810.

FIG. 1812A-B shows a nucleotide sequence (SEQ ID NO:1812) of a native sequence PRO38448 cDNA, wherein SEQ ID NO:1812 is a clone designated herein as “DNA227985”.

FIG. 1813 shows the amino acid sequence (SEQ ID NO:1813) derived from the coding sequence of SEQ ID NO:1812 shown in FIG. 1812.

FIG. 1814 shows a nucleotide sequence (SEQ ID NO:1814) of a native sequence PRO84003 cDNA, wherein SEQ ID NO:1814 is a clone designated herein as “DNA328092”.

FIG. 1815 shows the amino acid sequence (SEQ ID NO:1815) derived from the coding sequence of SEQ ID NO:1814 shown in FIG. 1814.

FIG. 1816 shows a nucleotide sequence (SEQ ID NO:1816) of a native sequence PRO81900 cDNA, wherein SEQ ID NO:1816 is a clone designated herein as “DNA325363”.

FIG. 1817 shows the amino acid sequence (SEQ ID NO:1817) derived from the coding sequence of SEQ ID NO:1817 shown in Figure

FIG. 1818 shows a nucleotide sequence (SEQ ID NO:1818) of a native sequence PRO23814 cDNA, wherein SEQ ID NO:1818 is a clone designated herein as “DNA329466”.

FIG. 1819 shows the amino acid sequence (SEQ ID NO:1819) derived from the coding sequence of SEQ ID NO:1818 shown in FIG. 1818.

FIG. 1820 shows a nucleotide sequence (SEQ ID NO:1820) of a native sequence PRO85033 cDNA, wherein SEQ ID NO:1820 is a clone designated herein as “DNA329467”.

FIG. 1821 shows the amino acid sequence (SEQ ID NO:1821) derived from the coding sequence of SEQ ID NO:1820 shown in FIG. 1820.

FIG. 1822 shows a nucleotide sequence (SEQ ID NO:1822) of a native sequence PRO88 cDNA, wherein SEQ ID NO:1822 is a clone designated herein as “DNA329468”.

FIG. 1823 shows the amino acid sequence (SEQ ID NO:1823) derived from the coding sequence of SEQ ID NO:1822 shown in FIG. 1822.

FIG. 1824A-B shows a nucleotide sequence (SEQ ID NO:1824) of a native sequence PRO24061 cDNA, wherein SEQ ID NO:1824 is a clone designated herein as “DNA194784”.

FIG. 1825 shows the amino acid sequence (SEQ ID NO:1825) derived from the coding sequence of SEQ ID NO:1824 shown in FIG. 1824.

FIG. 1826 shows a nucleotide sequence (SEQ ID NO:1826) of a native sequence PRO85034 cDNA, wherein SEQ ID NO:1826 is a clone designated herein as “DNA329469”.

FIG. 1827 shows the amino acid sequence (SEQ ID NO:1827) derived from the coding sequence of SEQ ID NO:1826 shown in FIG. 1826.

FIG. 1828 shows a nucleotide sequence (SEQ ID NO:1828) of a native sequence PRO85035 cDNA, wherein SEQ ID NO:1828 is a clone designated herein as “DNA329470”.

FIG. 1829 shows the amino acid sequence (SEQ ID NO:1829) derived from the coding sequence of SEQ ID NO:1828 shown in FIG. 1828.

FIG. 1830 shows a nucleotide sequence (SEQ ID NO:1830) of a native sequence PRO85036 cDNA, wherein SEQ ID NO:1830 is a clone designated herein as “DNA329471”.

FIG. 1831 shows the amino acid sequence (SEQ ID NO:1831) derived from the coding sequence of SEQ ID NO:1831 shown in Figure

FIG. 1832 shows a nucleotide sequence (SEQ ID NO:1832) of a native sequence PRO85037 cDNA, wherein SEQ ID NO:1832 is a clone designated herein as “DNA329472”.

FIG. 1833 shows the amino acid sequence (SEQ ID NO:1833) derived from the coding sequence of SEQ ID NO:1832 shown in FIG. 1832.

FIG. 1834 shows a nucleotide sequence (SEQ ID NO:1834) of a native sequence cDNA, wherein SEQ ID NO:1834 is a clone designated herein as “DNA136927”.

FIG. 1835 shows a nucleotide sequence (SEQ ID NO:1835) of a native sequence PRO1265 cDNA, wherein SEQ ID NO:1835 is a clone designated herein as “DNA304827”.

FIG. 1836 shows the amino acid sequence (SEQ ID NO:1836) derived from the coding sequence of SEQ ID NO:1835 shown in FIG. 1835.

FIG. 1837 shows a nucleotide sequence (SEQ ID NO:1837) of a native sequence PRO85038 cDNA, wherein SEQ ID NO:1837 is a clone designated herein as “DNA329473”.

FIG. 1838 shows the amino acid sequence (SEQ ID NO:1838) derived from the coding sequence of SEQ ID NO:1837 shown in FIG. 1837.

FIG. 1839 shows a nucleotide sequence (SEQ ID NO:1839) of a native sequence cDNA, wherein SEQ ID NO:1839 is a clone designated herein as “DNA195707”.

FIG. 1840 shows a nucleotide sequence (SEQ ID NO:1840) of a native sequence PRO38893 cDNA, wherein SEQ ID NO: is a clone designated herein as “DNA329474”.

FIG. 1841 shows the amino acid sequence (SEQ ID NO:1841) derived from the coding sequence of SEQ ID NO:1840 shown in FIG. 1840.

FIG. 1842 shows a nucleotide sequence (SEQ ID NO:1842) of a native sequence PRO85039 cDNA, wherein SEQ ID NO:1842 is a clone designated herein as “DNA329475”.

FIG. 1843 shows the amino acid sequence (SEQ ID NO:1843) derived from the coding sequence of SEQ ID NO:1843 shown in Figure

FIG. 1844A-B shows a nucleotide sequence (SEQ ID NO:1844) of a native sequence PRO85040 cDNA, wherein SEQ ID NO:1844 is a clone designated herein as “DNA329476”.

FIG. 1845 shows the amino acid sequence (SEQ ID NO:1845) derived from the coding sequence of SEQ ID NO:1845 shown in Figure

FIG. 1846 shows a nucleotide sequence (SEQ ID NO:1846) of a native sequence PRO51137 cDNA, wherein SEQ ID NO:1846 is a clone designated herein as “DNA256087”.

FIG. 1847 shows the amino acid sequence (SEQ ID NO:1847) derived from the coding sequence of SEQ ID NO:1846 shown in FIG. 1846.

FIG. 1848A-B shows a nucleotide sequence (SEQ ID NO:1848) of a native sequence PRO85041 cDNA, wherein SEQ ID NO:1848 is a clone designated herein as “DNA329477”.

FIG. 1849 shows the amino acid sequence (SEQ ID NO:1849) derived from the coding sequence of SEQ ID NO:1848 shown in FIG. 1848.

FIG. 1850 shows a nucleotide sequence (SEQ ID NO:1850) of a native sequence PRO10720 cDNA, wherein SEQ ID NO:1850 is a clone designated herein as “DNA329478”.

FIG. 1851 shows the amino acid sequence (SEQ ID NO:1851) derived from the coding sequence of SEQ ID NO:1850 shown in FIG. 1850.

FIG. 1852 shows a nucleotide sequence (SEQ ID NO:1852) of a native sequence cDNA, wherein SEQ ID NO:1852 is a clone designated herein as “DNA329479”.

FIG. 1853A-B shows a nucleotide sequence (SEQ ID NO:1853) of a native sequence PRO85043 cDNA, wherein SEQ ID NO:1853 is a clone designated herein as “DNA329480”.

FIG. 1854 shows the amino acid sequence (SEQ ID NO:1854) derived from the coding sequence of SEQ ID NO:1853 shown in FIG. 1853.

FIG. 1855 shows a nucleotide sequence (SEQ ID NO:1855) of a native sequence PRO60949 cDNA, wherein SEQ ID NO:1855 is a clone designated herein as “DNA329481”.

FIG. 1856 shows the amino acid sequence (SEQ ID NO:1856) derived from the coding sequence of SEQ ID NO:1855 shown in FIG. 1855.

FIG. 1857 shows a nucleotide sequence (SEQ ID NO:1857) of a native sequence PRO85044 cDNA, wherein SEQ ID NO:1857 is a clone designated herein as “DNA329482”.

FIG. 1858 shows the amino acid sequence (SEQ ID NO:1858) derived from the coding sequence of SEQ ID NO:1857 shown in FIG. 1857.

FIG. 1859 shows a nucleotide sequence (SEQ ID NO:1859) of a native sequence PRO20110 cDNA, wherein SEQ ID NO:1859 is a clone designated herein as “DNA329483”.

FIG. 1860 shows the amino acid sequence (SEQ ID NO:1860) derived from the coding sequence of SEQ ID NO:1859 shown in FIG. 1859.

FIG. 1861 shows a nucleotide sequence (SEQ ID NO:1861) of a native sequence cDNA, wherein SEQ ID NO:1861 is a clone designated herein as “DNA329484”.

FIG. 1862A-B shows a nucleotide sequence (SEQ ID NO:1862) of a native sequence PRO85046 cDNA, wherein SEQ ID NO:1862 is a clone designated herein as “DNA329485”.

FIG. 1863 shows the amino acid sequence (SEQ ID NO:1863) derived from the coding sequence of SEQ ID NO:1862 shown in FIG. 1862.

FIG. 1864 shows a nucleotide sequence (SEQ ID NO:1864) of a native sequence PRO84051 cDNA, wherein SEQ ID NO:1864 is a clone designated herein as “DNA328146”.

FIG. 1865 shows the amino acid sequence (SEQ ID NO:1865) derived from the coding sequence of SEQ ID NO:1864 shown in FIG. 1864.

FIG. 1866 shows a nucleotide sequence (SEQ ID NO:1866) of a native sequence PRO85047 cDNA, wherein SEQ ID NO:1866 is a clone designated herein as “DNA329486”.

FIG. 1867 shows the amino acid sequence (SEQ ID NO:1867) derived from the coding sequence of SEQ ID NO:1866 shown in FIG. 1866.

FIG. 1868A-B shows a nucleotide sequence (SEQ ID NO:1868) of a native sequence PRO85048 cDNA, wherein SEQ ID NO:1868 is a clone designated herein as “DNA329487”.

FIG. 1869 shows the amino acid sequence (SEQ ID NO:1869) derived from the coding sequence of SEQ ID NO:1868 shown in FIG. 1868

FIG. 1870 shows a nucleotide sequence (SEQ ID NO:1870) of a native sequence PRO85049 cDNA, wherein SEQ ID NO:1870 is a clone designated, herein as “DNA329488”.

FIG. 1871 shows the amino acid sequence (SEQ ID NO:1871) derived from the coding sequence of SEQ ID NO:1871 shown in Figure

FIG. 1872 shows a nucleotide sequence (SEQ ID NO:1872) of a native sequence PRO85050 cDNA, wherein SEQ ID NO:1872 is a clone designated herein as “DNA329489”.

FIG. 1873 shows the amino acid sequence (SEQ ID NO:1873) derived from the coding sequence of SEQ ID NO:1872 shown in FIG. 1872.

FIG. 1874 shows a nucleotide sequence (SEQ ID NO:1874) of a native sequence PRO70016 cDNA, wherein SEQ ID NO:1874 is a clone designated herein as “DNA288255”.

FIG. 1875 shows the amino acid sequence (SEQ ID NO:1875) derived from the coding sequence of SEQ ID NO:1874 shown in FIG. 1874.

FIG. 1876 shows a nucleotide sequence (SEQ ID NO:1876) of a native sequence PRO85051 cDNA, wherein SEQ ID NO:1876 is a clone designated herein as “DNA329490”.

FIG. 1877 shows the amino acid sequence (SEQ ID NO:1877) derived from the coding sequence of SEQ ID NO:1876 shown in FIG. 1876.

FIG. 1878 shows a nucleotide sequence (SEQ ID NO:1878) of a native sequence cDNA, wherein SEQ ID NO:1878 is a clone designated herein as “DNA259903”.

FIG. 1879 shows a nucleotide sequence (SEQ ID NO:1879) of a native sequence cDNA, wherein SEQ ID NO:1879 is a clone designated herein as “DNA259749”.

FIG. 1880 shows a nucleotide sequence (SEQ ID NO:1880) of a native sequence PRO85052 cDNA, wherein SEQ ID NO:1880 is a clone designated herein as “DNA329491”.

FIG. 1881 shows the amino acid sequence (SEQ ID NO:1881) derived from the coding sequence of SEQ ID NO:1880 shown in FIG. 1880.

FIG. 1882 shows a nucleotide sequence (SEQ ID NO:1882) of a native sequence PRO85053 cDNA, wherein SEQ ID NO:1882 is a clone designated herein as “DNA329492”.

FIG. 1883 shows the amino acid sequence (SEQ ID NO:1883) derived from the coding sequence of SEQ ID NO:1882 shown in FIG. 1882.

FIG. 1884 shows a nucleotide sequence (SEQ ID NO:1884) of a native sequence PRO85054 cDNA, wherein SEQ ID NO:1884 is a clone designated herein as “DNA329493”.

FIG. 1885 shows the amino acid sequence (SEQ ID NO:1885) derived from the coding sequence of SEQ ID NO:1884 shown in FIG. 1884.

FIG. 1886A-B shows a nucleotide sequence (SEQ ID NO:1886) of a native sequence PRO85055 cDNA, wherein SEQ ID NO:1886 is a clone designated herein as “DNA329494”.

FIG. 1887 shows the amino acid sequence (SEQ ID NO:1887) derived from the coding sequence of SEQ ID NO:1886 shown in FIG. 1886.

FIG. 1888 shows a nucleotide sequence (SEQ ID NO:1888) of a native sequence PRO52486 cDNA, wherein SEQ ID NO:1888 is a clone designated herein as “DNA257959”.

FIG. 1889 shows the amino acid sequence (SEQ ID NO:1889) derived from the coding sequence of SEQ ID NO:1888 shown in FIG. 1888.

FIG. 1890 shows a nucleotide sequence (SEQ ID NO:1890) of a native sequence PRO85056 cDNA, wherein SEQ ID NO:1890 is a clone designated herein as “DNA329495”.

FIG. 1891 shows the amino acid sequence (SEQ ID NO:1891) derived from the coding sequence of SEQ ID NO:1890 shown in FIG. 1890.

FIG. 1892 shows a nucleotide sequence (SEQ ID NO:1892) of a native sequence PRO85057 cDNA, wherein SEQ ID NO:1892 is a clone designated herein as “DNA329496”.

FIG. 1893 shows the amino acid sequence (SEQ ID NO:1893) derived from the coding sequence of SEQ ID NO:1892 shown in FIG. 1892.

FIG. 1894 shows a nucleotide sequence (SEQ ID NO:1894) of a native sequence PRO85058 cDNA, wherein SEQ ID NO:1894 is a clone designated herein as “DNA329497”.

FIG. 1895 shows the amino acid sequence (SEQ ID NO:1895) derived from the coding sequence of SEQ ID NO:1894 shown in FIG. 1894.

FIG. 1896 shows a nucleotide sequence (SEQ ID NO:1896) of a native sequence PRO85059 cDNA, wherein SEQ ID NO:1896 is a clone designated herein as “DNA329498”.

FIG. 1897 shows the amino acid sequence (SEQ ID NO:1897) derived from the coding sequence of SEQ ID NO:1896 shown in FIG. 1896.

FIG. 1898A-B shows a nucleotide sequence (SEQ ID NO:1898) of a native sequence PRO85060 cDNA, wherein SEQ ID NO:1898 is a clone designated herein as “DNA329499”.

FIG. 1899 shows the amino acid sequence (SEQ ID NO:1899) derived from the coding sequence of SEQ ID NO:1898 shown in FIG. 1898.

FIG. 1900 shows a nucleotide sequence (SEQ ID NO:1900) of a native sequence PRO85061 cDNA, wherein SEQ ID NO:1900 is a clone designated herein as “DNA329500”.

FIG. 1901 shows the amino acid sequence (SEQ ID NO:1901) derived from the coding sequence of SEQ ID NO:1900 shown in FIG. 1900.

FIG. 1902 shows a nucleotide sequence (SEQ ID NO:1902) of a native sequence PRO85062 cDNA, wherein SEQ ID NO:1902 is a clone designated herein as “DNA329501”.

FIG. 1903 shows the amino acid sequence (SEQ ID NO:1903) derived from the coding sequence of SEQ ID NO:1902 shown in FIG. 1902.

FIG. 1904 shows a nucleotide sequence (SEQ ID NO:1904) of a native sequence PRO85063 cDNA, wherein SEQ ID NO:1904 is a clone designated herein as “DNA329502”.

FIG. 1905 shows the amino acid sequence (SEQ ID NO:1905) derived from the coding sequence of SEQ ID NO:1904 shown in FIG. 1904.

FIG. 1906A-B shows a nucleotide sequence (SEQ ID NO:1906) of a native sequence cDNA, wherein SEQ ID NO:1906 is a clone designated herein as “DNA329503”.

FIG. 1907 shows a nucleotide sequence (SEQ ID NO:1907) of a native sequence PRO69635 cDNA, wherein SEQ ID NO:1907 is a clone designated herein as “DNA325417”.

FIG. 1908 shows the amino acid sequence (SEQ ID NO:1908) derived from the coding sequence of SEQ ID NO:1907 shown in FIG. 1907.

FIG. 1909 shows a nucleotide sequence (SEQ ID NO:1909) of a native sequence PRO85065 cDNA, wherein SEQ ID NO:1909 is a clone designated herein as “DNA329504”.

FIG. 1910 shows the amino acid sequence (SEQ ID NO:1910) derived from the coding sequence of SEQ ID NO:1909 shown in FIG. 1909.

FIG. 1911 shows a nucleotide sequence (SEQ ID NO:1911) of a native sequence cDNA, wherein SEQ ID NO:1911 is a clone designated herein as “DNA329505”.

FIG. 1912 shows a nucleotide sequence (SEQ ID NO:1912) of a native sequence PRO85067 cDNA, wherein SEQ ID NO:1912 is a clone designated herein as “DNA329506”.

FIG. 1913 shows the amino acid sequence (SEQ ID NO:1913) derived from the coding sequence of SEQ ID NO:1912 shown in FIG. 1912.

FIG. 1914 shows a nucleotide sequence (SEQ ID NO:1914) of a native sequence PRO85068 cDNA, wherein SEQ ID NO:1914 is a clone designated herein as “DNA329507”.

FIG. 1915 shows the amino acid sequence (SEQ ID NO:1915) derived from the coding sequence of SEQ ID NO:1914 shown in FIG. 1914.

FIG. 1916A-B shows a nucleotide sequence (SEQ ID NO:1916) of a native sequence PRO85069 cDNA, wherein SEQ ID NO:1916 is a clone designated herein as “DNA329508”.

FIG. 1917 shows the amino acid sequence (SEQ ID NO:1917) derived from the coding sequence of SEQ ID NO:1916 shown in FIG. 1916.

FIG. 1918 shows a nucleotide sequence (SEQ ID NO:1918) of a native sequence PRO85070 cDNA, wherein SEQ ID NO:1918 is a clone designated herein as “DNA329509”.

FIG. 1919 shows the amino acid sequence (SEQ ID NO:1919) derived from the coding sequence of SEQ ID NO:1918 shown in FIG. 1918.

FIG. 1920A-B shows a nucleotide sequence (SEQ ID NO:1920) of a native sequence cDNA, wherein SEQ ID NO:1920 is a clone designated herein as “DNA258863”.

FIG. 1921 shows a nucleotide sequence (SEQ ID NO:1921) of a native sequence PRO85071 cDNA, wherein SEQ ID NO:1921 is a clone designated herein as “DNA329510”.

FIG. 1922 shows the amino acid sequence (SEQ ID NO:1922) derived from the coding sequence of SEQ ID NO:1921 shown in FIG. 1921.

FIG. 1923 shows a nucleotide sequence (SEQ ID NO:1923) of a native sequence PRO23576 cDNA, wherein SEQ ID NO:1923 is a clone designated herein as “DNA194184”.

FIG. 1924 shows the amino acid sequence (SEQ ID NO:1924) derived from the coding sequence of SEQ ID NO:1923 shown in FIG. 1923.

FIG. 1925 shows a nucleotide sequence (SEQ ID NO:1925) of a native sequence PRO85072 cDNA, wherein SEQ ID NO:1925 is a clone designated herein as “DNA3295 11”.

FIG. 1926 shows the amino acid sequence (SEQ ID NO:1926) derived from the coding sequence of SEQ ID NO:1925 shown in FIG. 1925.

FIG. 1927 shows a nucleotide sequence (SEQ ID NO:1927) of a native sequence PRO84141 cDNA, wherein SEQ ID NO:1927 is a clone designated herein as “DNA328238”.

FIG. 1928 shows the amino acid sequence (SEQ ID NO:1928) derived from the coding sequence of SEQ ID NO:1927 shown in FIG. 1927.

FIG. 1929 shows a nucleotide sequence (SEQ ID NO:1929) of a native sequence PRO85073 cDNA, wherein SEQ ID NO:1929 is a clone designated herein as “DNA329512”.

FIG. 1930 shows the amino acid sequence (SEQ ID NO:1930) derived from the coding sequence of SEQ ID NO:1929 shown in FIG. 1929.

FIG. 1931 shows a nucleotide sequence (SEQ ID NO:1931) of a native sequence PRO85074 cDNA, wherein SEQ ID NO:1931 is a clone designated herein as “DNA329513”.

FIG. 1932 shows the amino acid sequence (SEQ ID NO:1932) derived from the coding sequence of SEQ ID NO:1931 shown in FIG. 1931.

FIG. 1933 shows a nucleotide sequence (SEQ ID NO:1933) of a native sequence PRO85075 cDNA, wherein SEQ ID NO:1933 is a clone designated herein as “DNA329514”.

FIG. 1934 shows the amino acid sequence (SEQ ID NO:1934) derived from the coding sequence of SEQ ID NO:1933 shown in FIG. 1933.

FIG. 1935 shows a nucleotide sequence (SEQ ID NO:1935) of a native sequence PRO4404 cDNA, wherein SEQ ID NO:1935 is a clone designated herein as “DNA84142”.

FIG. 1936 shows the amino acid sequence (SEQ ID NO:1936) derived from the coding sequence of SEQ ID NO:1935 shown in FIG. 1935.

FIG. 1937 shows a nucleotide sequence (SEQ ID NO:1937) of a native sequence PRO4348 cDNA, wherein SEQ ID NO:1937 is a clone designated herein as “DNA325654”.

FIG. 1938 shows the amino acid sequence (SEQ ID NO:1938) derived from the coding sequence of SEQ ID NO:1937 shown in FIG. 1937.

FIG. 1939 shows a nucleotide sequence (SEQ ID NO:1939) of a native sequence PRO4347 cDNA, wherein SEQ ID NO:1939 is a clone designated herein as “DNA329515”.

FIG. 1940 shows the amino acid sequence (SEQ ID NO:1940) derived from the coding sequence of SEQ ID NO:1940 shown in Figure

FIG. 1941 shows a nucleotide sequence (SEQ ID NO:1941) of a native sequence PRO220 cDNA, wherein SEQ ID NO:1941 is a clone designated herein as “DNA329516”.

FIG. 1942 shows the amino acid sequence (SEQ ID NO:1942) derived from the coding sequence of SEQ ID NO:1941 shown in FIG. 1941.

FIG. 1943 shows a nucleotide sequence (SEQ ID NO:1943) of a native sequence PRO85076 cDNA, wherein SEQ ID NO:1943 is a clone designated herein as “DNA329517”.

FIG. 1944 shows the amino acid sequence (SEQ ID NO:1944) derived from the coding sequence of SEQ ID NO:1943 shown in FIG. 1943.

FIG. 1945 shows a nucleotide sequence (SEQ ID NO:1945) of a native sequence PRO329 cDNA, wherein SEQ ID NO:1945 is a clone designated herein as “DNA323978”.

FIG. 1946 shows the amino acid sequence (SEQ ID NO:1946) derived from the coding sequence of SEQ ID NO:1945 shown in FIG. 1945.

FIG. 1947 shows a nucleotide sequence (SEQ ID NO:1947) of a native sequence PRO85077 cDNA, wherein SEQ ID NO:1947 is a clone designated herein as “DNA329518”.

FIG. 1948 shows the amino acid sequence (SEQ ID NO:1948) derived from the coding sequence of SEQ ID NO:1947 shown in FIG. 1947.

FIG. 1949 shows a nucleotide sequence (SEQ ID NO:1949) of a native sequence PRO38838 cDNA, wherein SEQ ID NO:1949 is a clone designated herein as “DNA233283”.

FIG. 1950 shows the amino acid sequence (SEQ ID NO:1950) derived from the coding sequence of SEQ ID NO:1949 shown in FIG. 1949.

FIG. 1951 shows a nucleotide sequence (SEQ ID NO:1951) of a native sequence PRO941 cDNA, wherein SEQ ID NO:1951 is a clone designated herein as “DNA329519”.

FIG. 1952 shows the amino acid sequence (SEQ ID NO:1952) derived from the coding sequence of SEQ ID NO:1951 shown in FIG. 1951.

FIG. 1953 shows a nucleotide sequence (SEQ ID NO:1953) of a native sequence PRO1054 cDNA, wherein SEQ ID NO:1953 is a clone designated herein as “DNA58853”.

FIG. 1954 shows the amino acid sequence (SEQ ID NO:1954) derived from the coding sequence of SEQ ID NO:1954 shown in Figure

FIG. 1955 shows a nucleotide sequence (SEQ ID NO:1955) of a native sequence PRO85078 cDNA, wherein SEQ ID NO:1955 is a clone designated herein as “DNA329520”.

FIG. 1956 shows the amino acid sequence (SEQ ID NO:1956) derived from the coding sequence of SEQ ID NO:1955 shown in FIG. 1955.

FIG. 1957 shows a nucleotide sequence (SEQ ID NO:1957) of a native sequence PRO6517 cDNA, wherein SEQ ID NO:1957 is a clone designated herein as “DNA109234”.

FIG. 1958 shows the amino acid sequence (SEQ ID NO:1958) derived from the coding sequence of SEQ ID NO:1957 shown in FIG. 1957.

FIG. 1959 shows a nucleotide sequence (SEQ ID NO:1959) of a native sequence PRO85079 cDNA, wherein SEQ ID NO:1959 is a clone designated herein as “DNA329521”.

FIG. 1960 shows the amino acid sequence (SEQ ID NO:1960) derived from the coding sequence of SEQ ID NO:1959 shown in FIG. 1959.

FIG. 1961 shows a nucleotide sequence (SEQ ID NO:1961) of a native sequence PRO12810 cDNA, wherein SEQ ID NO:1961 is a clone designated herein as “DNA150823”.

FIG. 1962 shows the amino acid sequence (SEQ ID NO:1962) derived from the coding sequence of SEQ ID NO:1961 shown in FIG. 1961.

FIG. 1963A-B shows a nucleotide sequence (SEQ ID NO:1963) of a native sequence PRO2598 cDNA, wherein SEQ ID NO:1963 is a clone designated herein as “DNA83118”.

FIG. 1964 shows the amino acid sequence (SEQ ID NO:1964) derived from the coding sequence of SEQ ID NO:1963 shown in FIG. 1963.

FIG. 1965 shows a nucleotide sequence (SEQ ID NO:1965) of a native sequence PRO2844 cDNA, wherein SEQ ID NO:1965 is a clone designated herein as “DNA88567”.

FIG. 1966 shows the amino acid sequence (SEQ ID NO:1966) derived from the coding sequence of SEQ ID NO:1965 shown in FIG. 1965.

FIG. 1967 shows a nucleotide sequence (SEQ ID NO:1967) of a native sequence PRO2852 cDNA, wherein SEQ ID NO:1967 is a clone designated herein as “DNA88583”.

FIG. 1968 shows the amino acid sequence (SEQ ID NO:1968) derived from the coding sequence of SEQ ID NO:1967 shown in FIG. 1967.

FIG. 1969 shows a nucleotide sequence (SEQ ID NO:1969) of a native sequence PRO24845 cDNA, wherein SEQ ID NO:1969 is a clone designated herein as “DNA196337”.

FIG. 1970 shows the amino acid sequence (SEQ ID NO:1970) derived from the coding sequence of SEQ ID NO:1969 shown in FIG. 1969.

FIG. 1971 shows a nucleotide sequence (SEQ ID NO:1971) of a native sequence PRO85080 cDNA, wherein SEQ ID NO:1971 is a clone designated herein as “DNA329522”.

FIG. 1972 shows the amino acid sequence (SEQ ID NO:1972) derived from the coding sequence of SEQ ID NO:1971 shown in FIG. 1971.

FIG. 1973 shows a nucleotide sequence (SEQ ID NO:1973) of a native sequence PRO2155 cDNA, wherein SEQ ID NO:1973 is a clone designated herein as “DNA329523”.

FIG. 1974 shows the amino acid sequence (SEQ ID NO:1974) derived from the coding sequence of SEQ ID NO:1973 shown in FIG. 1973.

FIG. 1975 shows a nucleotide sequence (SEQ ID NO:1975) of a native sequence PRO36996 cDNA, wherein SEQ ID NO:1975 is a clone designated herein as “DNA329524”.

FIG. 1976 shows the amino acid sequence (SEQ ID NO:1976) derived from the coding sequence of SEQ ID NO:1975 shown in FIG. 1975.

FIG. 1977 shows a nucleotide sequence (SEQ ID NO:1977) of a native sequence PRO2663 cDNA, wherein SEQ ID NO:1977 is a clone designated herein as “DNA88119”.

FIG. 1978 shows the amino acid sequence (SEQ ID NO:1978) derived from the coding sequence of SEQ ID NO:1977 shown in FIG. 1977.

FIG. 1979 shows a nucleotide sequence (SEQ ID NO:1979) of a native sequence PRO21942 cDNA, wherein SEQ ID NO:1979 is a clone designated herein as “DNA188234”.

FIG. 1980 shows the amino acid sequence (SEQ ID NO:1980) derived from the coding sequence of SEQ ID NO:1979 shown in FIG. 1979.

FIG. 1981 shows a nucleotide sequence (SEQ ID NO:1981) of a native sequence PRO36456 cDNA, wherein SEQ ID NO:1981 is a clone designated herein as “DNA225993”.

FIG. 1982 shows the amino acid sequence (SEQ ID NO:1982) derived from the coding sequence of SEQ ID NO:1981 shown in FIG. 1981.

FIG. 1983 shows a nucleotide sequence (SEQ ID NO:1983) of a native sequence PRO2590 cDNA, wherein SEQ ID NO:1983 is a clone designated herein as “DNA83101”.

FIG. 1984 shows the amino acid sequence (SEQ ID NO:1984) derived from the coding sequence of SEQ ID NO:1983 shown in FIG. 1983.

FIG. 1985 shows a nucleotide sequence (SEQ ID NO:1985) of a native sequence PRO69 cDNA, wherein SEQ ID NO:1985 is a clone designated herein as “DNA36714”.

FIG. 1986 shows the amino acid sequence (SEQ ID NO:1986) derived from the coding sequence of SEQ ID NO:1985 shown in FIG. 1985.

FIG. 1987 shows a nucleotide sequence (SEQ ID NO:1987) of a native sequence PRO36659 cDNA, wherein SEQ ID NO:1987 is a clone designated herein as “DNA226196”.

FIG. 1988 shows the amino acid sequence (SEQ ID NO:1988) derived from the coding sequence of SEQ ID NO:1987 shown in FIG. 1987.

FIG. 1989 shows a nucleotide sequence (SEQ ID NO:1989) of a native sequence PRO20139 cDNA, wherein SEQ ID NO:1989 is a clone designated herein as “DNA246375”.

FIG. 1990 shows the amino acid sequence (SEQ ID NO:1990) derived from the coding sequence of SEQ ID NO:1989 shown in FIG. 1989.

FIG. 1991 shows a nucleotide sequence (SEQ ID NO:1991) of a native sequence PRO2691 cDNA, wherein SEQ ID NO:1991 is a clone designated herein as “DNA88191”.

FIG. 1992 shows the amino acid sequence (SEQ ID NO:1992) derived from the coding sequence of SEQ ID NO:1991 shown in FIG. 1991.

FIG. 1993 shows a nucleotide sequence (SEQ ID NO:1993) of a native sequence PRO85081 cDNA, wherein SEQ ID NO:1993 is a clone designated herein as “DNA329525”.

FIG. 1994 shows the amino acid sequence (SEQ ID NO:1994) derived from the coding sequence of SEQ ID NO:1993 shown in FIG. 1993.

FIG. 1995 shows a nucleotide sequence (SEQ ID NO:1995) of a native sequence PRO4940 cDNA, wherein SEQ ID NO:1995 is a clone designated herein as “DNA328576”.

FIG. 1996 shows the amino acid sequence (SEQ ID NO:1996) derived from the coding sequence of SEQ ID NO:1995 shown in FIG. 1995.

FIG. 1997 shows a nucleotide sequence (SEQ ID NO:1997) of a native sequence PRO37421 cDNA, wherein SEQ ID NO:1997 is a clone designated herein as “DNA226958”.

FIG. 1998 shows the amino acid sequence (SEQ ID NO:1998) derived from the coding sequence of SEQ ID NO:1997 shown in FIG. 1997.

FIG. 1999 shows a nucleotide sequence (SEQ ID NO:1999) of a native sequence PRO81141 cDNA, wherein SEQ ID NO:1999 is a clone designated herein as “DNA324480”.

FIG. 2000 shows the amino acid sequence (SEQ ID NO:2000) derived from the coding sequence of SEQ ID NO:1999 shown in FIG. 1999.

FIG. 2001 shows a nucleotide sequence (SEQ ID NO:2001) of a native sequence PRO1718 cDNA, wherein SEQ ID NO:2001 is a clone designated herein as “DNA82362”.

FIG. 2002 shows the amino acid sequence (SEQ ID NO:2002) derived from the coding sequence of SEQ ID NO:2001 shown in FIG. 2001.

FIG. 2003 shows a nucleotide sequence (SEQ ID NO:2003) of a native sequence PRO37476 cDNA, wherein SEQ ID NO:2003 is a clone designated herein as “DNA227013”.

FIG. 2004 shows the amino acid sequence (SEQ ID NO:2004) derived from the coding sequence of SEQ ID NO:2003 shown in FIG. 2003.

FIG. 2005A-B shows a nucleotide sequence (SEQ ID NO:2005) of a native sequence PRO36827 cDNA, wherein SEQ ID NO:2005 is a clone designated herein as “DNA226364”.

FIG. 2006 shows the amino acid sequence (SEQ ID NO:2006) derived from the coding sequence of SEQ ID NO:2005 shown in FIG. 2005.

FIG. 2007 shows a nucleotide sequence (SEQ ID NO:2007) of a native sequence PRO2640 cDNA, wherein SEQ ID NO:2007 is a clone designated herein as “DNA88076”.

FIG. 2008 shows the amino acid sequence (SEQ ID NO:2008) derived from the coding sequence of SEQ ID NO:2007 shown in FIG. 2007.

FIG. 2009 shows a nucleotide sequence (SEQ ID NO:2009) of a native sequence PRO2013 cDNA, wherein SEQ ID NO:2009 is a clone designated herein as “DNA75526”.

FIG. 2010 shows the amino acid sequence (SEQ ID NO:2010) derived from the coding sequence of SEQ ID NO:2009 shown in FIG. 2009.

FIG. 2011 shows a nucleotide sequence (SEQ ID NO:2011) of a native sequence PRO2177 cDNA, wherein SEQ ID NO:2011 is a clone designated herein as “DNA88116”.

FIG. 2012 shows the amino acid sequence (SEQ ID NO:2012) derived from the coding sequence of SEQ ID NO:2011 shown in FIG. 2011.

FIG. 2013 shows a nucleotide sequence (SEQ ID NO:2013) of a native sequence PRO4695 cDNA, wherein SEQ ID NO:2013 is a clone designated herein as “DNA226380”.

FIG. 2014 shows the amino acid sequence (SEQ ID NO:2014) derived from the coding sequence of SEQ ID NO:2013 shown in FIG. 2013.

FIG. 2015 shows a nucleotide sequence (SEQ ID NO:2015) of a native sequence PRO80473 cDNA, wherein SEQ ID NO:2015 is a clone designated herein as “DNA329526”.

FIG. 2016 shows the amino acid sequence (SEQ ID NO:2016) derived from the coding sequence of SEQ ID NO:2015 shown in FIG. 2015.

FIG. 2017A-B shows a nucleotide sequence (SEQ ID NO:2017) of a native sequence PRO2249 cDNA, wherein SEQ ID NO:2017 is a clone designated herein as “DNA88251”.

FIG. 2018 shows the amino acid sequence (SEQ ID NO:2018) derived from the coding sequence of SEQ ID NO:2017 shown in FIG. 2017.

FIG. 2019A-B shows a nucleotide sequence (SEQ ID NO:2019) of a native sequence PRO25018 cDNA, wherein SEQ ID NO:2019 is a clone designated herein as “DNA196533”.

FIG. 2020 shows the amino acid sequence (SEQ ID NO:2020) derived from the coding sequence of SEQ ID NO:2019 shown in FIG. 2019.

FIG. 2021 shows a nucleotide sequence (SEQ ID NO:2021) of a native sequence PRO36124 cDNA, wherein SEQ ID NO:2021 is a clone designated herein as “DNA225661”.

FIG. 2022 shows the amino acid sequence (SEQ ID NO:2022) derived from the coding sequence of SEQ ID NO:2021 shown in FIG. 2021.

FIG. 2023 shows a nucleotide sequence (SEQ ID NO:2023) of a native sequence PRO2868 cDNA, wherein SEQ ID NO:2023 is a clone designated herein as “DNA329527”.

FIG. 2024 shows the amino acid sequence (SEQ ID NO:2024) derived from the coding sequence of SEQ ID NO:2023 shown in FIG. 2023.

FIG. 2025 shows a nucleotide sequence (SEQ ID NO:2025) of a native sequence PRO85082 cDNA, wherein SEQ ID NO:2025 is a clone designated herein as “DNA329528”.

FIG. 2026 shows the amino acid sequence (SEQ ID NO:2026) derived from the coding sequence of SEQ ID NO:2025 shown in FIG. 2025.

FIG. 2027 shows a nucleotide sequence (SEQ ID NO:2027) of a native sequence PRO85083 cDNA, wherein SEQ ID NO:2027 is a clone designated herein as “DNA329529”.

FIG. 2028 shows the amino acid sequence (SEQ ID NO:2028) derived from the coding sequence of SEQ ID NO:2028 shown in Figure

FIG. 2029 shows a nucleotide sequence (SEQ ID NO:2029) of a native sequence PRO82739 cDNA, wherein SEQ ID NO:2029 is a clone designated herein as “DNA326343”.

FIG. 2030 shows the amino acid sequence (SEQ ID NO:2030) derived from the coding sequence of SEQ ID NO:2029 shown in FIG. 2029.

FIG. 2031 shows a nucleotide sequence (SEQ ID NO:2031) of a native sequence PRO2809 cDNA, wherein SEQ ID NO:2031 is a clone designated herein as “DNA88472”.

FIG. 2032 shows the amino acid sequence (SEQ ID NO:2032) derived from the coding sequence of SEQ ID NO:2031 shown in FIG. 2031.

FIG. 2033 shows a nucleotide sequence (SEQ ID NO:2033) of a native sequence PRO11604 cDNA, wherein SEQ ID NO:2033 is a clone designated herein as “DNA329530”.

FIG. 2034 shows the amino acid sequence (SEQ ID NO:2034) derived from the coding sequence of SEQ ID NO:2033 shown in FIG. 2033.

FIG. 2035 shows a nucleotide sequence (SEQ ID NO:2035) of a native sequence PRO12452 cDNA, wherein SEQ ID NO:2035 is a clone designated herein as “DNA150757”.

FIG. 2036 shows the amino acid sequence (SEQ ID NO:2036) derived from the coding sequence of SEQ ID NO:2035 shown in FIG. 2035.

FIG. 2037 shows a nucleotide sequence (SEQ ID NO:2037) of a native sequence PRO21783 cDNA, wherein SEQ ID NO:2037 is a clone designated herein as “DNA188330”.

FIG. 2038 shows the amino acid sequence (SEQ ID NO:2038) derived from the coding sequence of SEQ ID NO:2037 shown in FIG. 2037.

FIG. 2039 shows a nucleotide sequence (SEQ ID NO:2039) of a native sequence PRO84151 cDNA, wherein SEQ ID NO:2039 is a clone designated herein as “DNA328258”.

FIG. 2040 shows the amino acid sequence (SEQ ID NO:2040) derived from the coding sequence of SEQ ID NO:2039 shown in FIG. 2039.

FIG. 2041 shows a nucleotide sequence (SEQ ID NO:2041) of a native sequence PRO85084 cDNA, wherein SEQ ID NO:2041 is a clone designated herein as “DNA329531”.

FIG. 2042 shows the amino acid sequence (SEQ ID NO:2042) derived from the coding sequence of SEQ ID NO:2041 shown in FIG. 2041.

FIG. 2043 shows a nucleotide sequence (SEQ ID NO:2043) of a native sequence PRO37029 cDNA, wherein SEQ ID NO:2043 is a clone designated herein as “DNA329007”.

FIG. 2044 shows the amino acid sequence (SEQ ID NO:2044) derived from the coding sequence of SEQ ID NO:2043 shown in FIG. 2043.

FIG. 2045 shows a nucleotide sequence (SEQ ID NO:2045) of a native sequence PRO36606 cDNA, wherein SEQ ID NO:2045 is a clone designated herein as “DNA226143”.

FIG. 2046 shows the amino acid sequence (SEQ ID NO:2046) derived from the coding sequence of SEQ ID NO:2045 shown in FIG. 2045.

FIG. 2047 shows a nucleotide sequence (SEQ ID NO:2047) of a native sequence PRO24862 cDNA, wherein SEQ ID NO:2047 is a clone designated herein as “DNA196357”.

FIG. 2048 shows the amino acid sequence (SEQ ID NO:2048) derived from the coding sequence of SEQ ID NO:2047 shown in FIG. 2047.

FIG. 2049A-B shows a nucleotide sequence (SEQ ID NO:2049) of a native sequence PRO37968 cDNA, wherein SEQ ID NO:2049 is a clone designated herein as “DNA227505”.

FIG. 2050 shows the amino acid sequence (SEQ ID NO:2050) derived from the coding sequence of SEQ ID NO:2049 shown in FIG. 2049.

FIG. 2051 shows a nucleotide sequence (SEQ ID NO:2051) of a native sequence PRO12658 cDNA, wherein SEQ ID NO:2051 is a clone designated herein as “DNA329532”.

FIG. 2052 shows the amino acid sequence (SEQ ID NO:2052) derived from the coding sequence of SEQ ID NO:2051 shown in FIG. 2051.

FIG. 2053 shows a nucleotide sequence (SEQ ID NO:2053) of a native sequence PRO85085 cDNA, wherein SEQ ID NO:2053 is a clone designated herein as “DNA329533”.

FIG. 2054 shows the amino acid sequence (SEQ ID NO:2054) derived from the coding sequence of SEQ ID NO:2053 shown in FIG. 2053.

FIG. 2055A-B shows a nucleotide sequence (SEQ ID NO:2055) of a native sequence PRO36420 cDNA, wherein SEQ ID NO:2055 is a clone designated herein as “DNA225957”.

FIG. 2056 shows the amino acid sequence (SEQ ID NO:2056) derived from the coding sequence of SEQ ID NO:2055 shown in FIG. 2055.

FIG. 2057 shows a nucleotide sequence (SEQ ID NO:2057) of a native sequence PRO84153 cDNA, wherein SEQ ID NO:2057 is a clone designated herein as “DNA328262”.

FIG. 2058 shows the amino acid sequence (SEQ ID NO:2058) derived from the coding sequence of SEQ ID NO:2057 shown in FIG. 2057.

FIG. 2059 shows a nucleotide sequence (SEQ ID NO:2059) of a native sequence PRO2904 cDNA, wherein SEQ ID NO:2059 is a clone designated herein as “DNA329534”.

FIG. 2060 shows the amino acid sequence (SEQ ID NO:2060) derived from the coding sequence of SEQ ID NO:2059 shown in FIG. 2059.

FIG. 2061 shows a nucleotide sequence (SEQ ID NO:2061) of a native sequence PRO85086 cDNA, wherein SEQ ID NO:2061 is a clone designated herein as “DNA329535”.

FIG. 2062 shows the amino acid sequence (SEQ ID NO:2062) derived from the coding sequence of SEQ ID NO:2061 shown in FIG. 2061.

FIG. 2063 shows a nucleotide sequence (SEQ ID NO:2063) of a native sequence PRO2733 cDNA, wherein SEQ ID NO:2063 is a clone designated herein as “DNA325039”.

FIG. 2064 shows the amino acid sequence (SEQ ID NO:2064) derived from the coding sequence of SEQ ID NO:2063 shown in FIG. 2063.

FIG. 2065 shows a nucleotide sequence (SEQ ID NO:2065) of a native sequence PRO23370 cDNA, wherein SEQ ID NO:2065 is a clone designated herein as “DNA329010”.

FIG. 2066 shows the amino acid sequence (SEQ ID NO:2066) derived from the coding sequence of SEQ ID NO:2065 shown in FIG. 2065.

FIG. 2067 shows a nucleotide sequence (SEQ ID NO:2067) of a native sequence PRO2602 cDNA, wherein SEQ ID NO:2067 is a clone designated herein as “DNA83134”.

FIG. 2068 shows the amino acid sequence (SEQ ID NO:2068) derived from the coding sequence of SEQ ID NO:2067 shown in FIG. 2067.

FIG. 2069 shows a nucleotide sequence (SEQ ID NO:2069) of a native sequence PRO4599 cDNA, wherein SEQ ID NO:2069 is a clone designated herein as “DNA103269”.

FIG. 2070 shows the amino acid sequence (SEQ ID NO:2070) derived from the coding sequence of SEQ ID NO:2069 shown in FIG. 2069.

FIG. 2071A-B shows a nucleotide sequence (SEQ ID NO:2071) of a native sequence PRO22775 cDNA, wherein SEQ ID NO:2071 is a clone designated herein as “DNA329536”.

FIG. 2072 shows the amino acid sequence (SEQ ID NO:2072) derived from the coding sequence of SEQ ID NO:2072 shown in FIG. 2072.

FIG. 2073 shows a nucleotide sequence (SEQ ID NO:2073) of a native sequence PRO85087 cDNA, wherein SEQ ID NO:2073 is a clone designated herein as “DNA329537”.

FIG. 2074 shows the amino acid sequence (SEQ ID NO:2074) derived from the coding sequence of SEQ ID NO:2073 shown in FIG. 2073.

FIG. 2075 shows a nucleotide sequence (SEQ ID NO:2075) of a native sequence PRO36963 cDNA, wherein SEQ ID NO:2075 is a clone designated herein as “DNA226500”.

FIG. 2076 shows the amino acid sequence (SEQ ID NO:2076) derived from the coding sequence of SEQ ID NO:2075 shown in FIG. 2075.

FIG. 2077 shows a nucleotide sequence (SEQ ID NO:2077) of a native sequence PRO20128 cDNA, wherein SEQ ID NO:2077 is a clone designated herein as “DNA329013”.

FIG. 2078 shows the amino acid sequence (SEQ ID NO:2078) derived from the coding sequence of SEQ ID NO:2077 shown in FIG. 2077.

FIG. 2079 shows a nucleotide sequence (SEQ ID NO:2079) of a native sequence PRO38443 cDNA, wherein SEQ ID NO:2079 is a clone designated herein as “DNA227980”.

FIG. 2080 shows the amino acid sequence (SEQ ID NO:2080) derived from the coding sequence of SEQ ID NO:2079 shown in FIG. 2079.

FIG. 2081 shows a nucleotide sequence (SEQ ID NO:2081) of a native sequence PRO85088 cDNA, wherein SEQ ID NO:2081 is a clone designated herein as “DNA329538”.

FIG. 2082 shows the amino acid sequence (SEQ ID NO:2082) derived from the coding sequence of SEQ ID NO:2081 shown in FIG. 2081.

FIG. 2083 shows a nucleotide sequence (SEQ ID NO:2083) of a native sequence PRO6180 cDNA, wherein SEQ ID NO:2083 is a clone designated herein as “DNA287376”.

FIG. 2084 shows the amino acid sequence (SEQ ID NO:2084) derived from the coding sequence of SEQ ID NO:2083 shown in FIG. 2083.

FIG. 2085 shows a nucleotide sequence (SEQ ID NO:2085) of a native sequence PRO85089 cDNA, wherein SEQ ID NO:2085 is a clone designated herein as “DNA329539”.

FIG. 2086 shows the amino acid sequence (SEQ ID NO:2086) derived from the coding sequence of SEQ ID NO:2085 shown in FIG. 2085.

FIG. 2087 shows a nucleotide sequence (SEQ ID NO:2087) of a native sequence PRO2520 cDNA, wherein SEQ ID NO:2087 is a clone designated herein as “DNA28759”.

FIG. 2088 shows the amino acid sequence (SEQ ID NO:2088) derived from the coding sequence of SEQ ID NO:2087 shown in FIG. 2087.

FIG. 2089 shows a nucleotide sequence (SEQ ID NO:2089) of a native sequence PRO4887 cDNA, wherein SEQ ID NO:2089 is a clone designated herein as “DNA329016”.

FIG. 2090 shows the amino acid sequence (SEQ ID NO:2090) derived from the coding sequence of SEQ ID NO:2089 shown in FIG. 2089.

FIG. 2091 shows a nucleotide sequence (SEQ ID NO:2091) of a native sequence PRO85090 cDNA, wherein SEQ ID NO:2091 is a clone designated herein as “DNA329540”.

FIG. 2092 shows the amino acid sequence (SEQ ID NO:2092) derived from the coding sequence of SEQ ID NO:2091 shown in FIG. 2091.

FIG. 2093 shows a nucleotide sequence (SEQ ID NO:2093) of a native sequence PRO4515 cDNA, wherein SEQ ID NO:2093 is a clone designated herein as “DNA93439”.

FIG. 2094 shows the amino acid sequence (SEQ ID NO:2094) derived from the coding sequence of SEQ ID NO:2093 shown in FIG. 2093.

FIG. 2095 shows a nucleotide sequence (SEQ ID NO:2095) of a native sequence PRO12358 cDNA, wherein SEQ ID NO:2095 is a clone designated herein as “DNA329541”.

FIG. 2096 shows the amino acid sequence (SEQ ID NO:2096) derived from the coding sequence of SEQ ID NO:2095 shown in FIG. 2095.

FIG. 2097 shows a nucleotide sequence (SEQ ID NO:2097) of a native sequence PRO37975 cDNA, wherein SEQ ID NO:2097 is a clone designated herein as “DNA227512”.

FIG. 2098 shows the amino acid sequence (SEQ ID NO:2098) derived from the coding sequence of SEQ ID NO:2097 shown in FIG. 2097

FIG. 2099 shows a nucleotide sequence (SEQ ID NO:2099) of a native sequence PRO12468 cDNA, wherein SEQ ID NO:2099 is a clone designated herein as “DNA324897”.

FIG. 2100 shows the amino acid sequence (SEQ ID NO:2100) derived from the coding sequence of SEQ ID NO:2099 shown in FIG. 2099.

FIG. 2101 shows a nucleotide sequence (SEQ ID NO:2101) of a native sequence PRO4767 cDNA, wherein SEQ ID NO:2101 is a clone designated herein as “DNA103440”.

FIG. 2102 shows the amino acid sequence (SEQ ID NO:2102) derived from the coding sequence of SEQ ID NO:2101 shown in FIG. 2101.

FIG. 2103 shows a nucleotide sequence (SEQ ID NO:2103) of a native sequence PRO4735 cDNA, wherein SEQ ID NO:2103 is a clone designated herein as “DNA329542”.

FIG. 2104 shows the amino acid sequence (SEQ ID NO:2104) derived from the coding sequence of SEQ ID NO:2103 shown in FIG. 2103.

FIG. 2105 shows a nucleotide sequence (SEQ ID NO:2105) of a native sequence PRO36506 cDNA, wherein SEQ ID NO:2105 is a clone designated herein as “DNA226043”.

FIG. 2106 shows the amino acid sequence (SEQ ID NO:2106) derived from the coding sequence of SEQ ID NO:2105 shown in FIG. 2105.

FIG. 2107 shows a nucleotide sequence (SEQ ID NO:2107) of a native sequence PRO2690 cDNA, wherein SEQ ID NO:2107 is a clone designated herein as “DNA88189”.

FIG. 2108 shows the amino acid sequence (SEQ ID NO:2108) derived from the coding sequence of SEQ ID NO:2107 shown in FIG. 2107.

FIG. 2109 shows a nucleotide sequence (SEQ ID NO:2109) of a native sequence PRO4808 cDNA, wherein SEQ ID NO:2109 is a clone designated herein as “DNA103481”.

FIG. 2110 shows the amino acid sequence (SEQ ID NO:2110) derived from the coding sequence of SEQ ID NO:2109 shown in FIG. 2109.

FIG. 2111 shows a nucleotide sequence (SEQ ID NO:2111) of a native sequence PRO4419 cDNA, wherein SEQ ID NO:2111 is a clone designated herein as “DNA329543”.

FIG. 2112 shows the amino acid sequence (SEQ ID NO:2112) derived from the coding sequence of SEQ ID NO:2111 shown in FIG. 2111.

FIG. 2113 shows a nucleotide sequence (SEQ ID NO:2113) of a native sequence PRO619 cDNA, wherein SEQ ID NO:2113 is a clone designated herein as “DNA329544”.

FIG. 2114 shows the amino acid sequence (SEQ ID NO:2114) derived from the coding sequence of SEQ ID NO:2113 shown in FIG. 2113.

FIG. 2115 shows a nucleotide sequence (SEQ ID NO:2115) of a native sequence PRO85091 cDNA, wherein SEQ ID NO:2115 is a clone designated herein as “DNA329545”.

FIG. 2116 shows the amino acid sequence (SEQ ID NO:2116) derived from the coding sequence of SEQ ID NO:2115 shown in FIG. 2115.

FIG. 2117 shows a nucleotide sequence (SEQ ID NO:2117) of a native sequence PRO23859 cDNA, wherein SEQ ID NO:2117 is a clone designated herein as “DNA328957”.

FIG. 2118 shows the amino acid sequence (SEQ ID NO:2118) derived from the coding sequence of SEQ ID NO:2117 shown in FIG. 2117.

FIG. 2119 shows a nucleotide sequence (SEQ ID NO:2119) of a native sequence PRO37696 cDNA, wherein SEQ ID NO:2119 is a clone designated herein as “DNA227233”.

FIG. 2120 shows the amino acid sequence (SEQ ID NO:2120) derived from the coding sequence of SEQ ID NO:2119 shown in FIG. 2119.

FIG. 2121 shows a nucleotide sequence (SEQ ID NO:2121) of a native sequence PRO296 cDNA, wherein SEQ ID NO:2121 is a clone designated herein as “DNA329546”.

FIG. 2122 shows the amino acid sequence (SEQ ID NO:2122) derived from the coding sequence of SEQ ID NO:2123 shown in FIG. 2123.

FIG. 2123 shows a nucleotide sequence (SEQ ID NO:2123) of a native sequence PRO34477 cDNA, wherein SEQ ID NO:2123 is a clone designated herein as “DNA218845”.

FIG. 2124 shows the amino acid sequence (SEQ ID NO:2124) derived from the coding sequence of SEQ ID NO:2123 shown in FIG. 2123.

FIG. 2125 shows a nucleotide sequence (SEQ ID NO:2125) of a native sequence PRO85092 cDNA, wherein SEQ ID NO:2125 is a clone designated herein as “DNA329547”.

FIG. 2126 shows the amino acid sequence (SEQ ID NO:2126) derived from the coding sequence of SEQ ID NO:2125 shown in FIG. 2125.

FIG. 2127 shows a nucleotide sequence (SEQ ID NO:2127) of a native sequence PRO24955 cDNA, wherein SEQ ID NO:2127 is a clone designated herein as “DNA196460”.

FIG. 2128 shows the amino acid sequence (SEQ ID NO:2128) derived from the coding sequence of SEQ ID NO:2127 shown in FIG. 2127.

FIG. 2129 shows a nucleotide sequence (SEQ ID NO:2129) of a native sequence PRO33679 cDNA, wherein SEQ ID NO:2129 is a clone designated herein as “DNA210134”.

FIG. 2130 shows the amino acid sequence (SEQ ID NO:2130) derived from the coding sequence of SEQ ID NO:2129 shown in FIG. 2129.

FIG. 2131 shows a nucleotide sequence (SEQ ID NO:2131) of a native sequence PRO36002 cDNA, wherein SEQ ID NO:2131 is a clone designated herein as “DNA225539”.

FIG. 2132 shows the amino acid sequence (SEQ ID NO:2132) derived from the coding sequence of SEQ ID NO:2131 shown in FIG. 2131.

FIG. 2133 shows a nucleotide sequence (SEQ ID NO:2133) of a native sequence PRO85093 cDNA, wherein SEQ ID NO:2133 is a clone designated herein as “DNA329548”.

FIG. 2134 shows the amino acid sequence (SEQ ID NO:2134) derived from the coding sequence of SEQ ID NO:2133 shown in FIG. 2133.

FIG. 2135 shows a nucleotide sequence (SEQ ID NO:2135) of a native sequence PRO71042 cDNA, wherein SEQ ID NO:2135 is a clone designated herein as “DNA304464”.

FIG. 2136 shows the amino acid sequence (SEQ ID NO:2136) derived from the coding sequence of SEQ ID NO:2135 shown in FIG. 2135.

FIG. 2137 shows a nucleotide sequence (SEQ ID NO:2137) of a native sequence PRO85094 cDNA, wherein SEQ ID NO:2137 is a clone designated herein as “DNA329549”.

FIG. 2138 shows the amino acid sequence (SEQ ID NO:2138) derived from the coding sequence of SEQ ID NO:2137 shown in FIG. 2137.

FIG. 2139 shows a nucleotide sequence (SEQ ID NO:2139) of a native sequence PRO36113 cDNA, wherein SEQ ID NO:2139 is a clone designated herein as “DNA225650”.

FIG. 2140 shows the amino acid sequence (SEQ ID NO:2140) derived from the coding sequence of SEQ ID NO:2139 shown in FIG. 2139.

FIG. 2141 shows a nucleotide sequence (SEQ ID NO:2141) of a native sequence PRO85095 cDNA, wherein SEQ ID NO:2141 is a clone designated herein as “DNA329550”.

FIG. 2142 shows the amino acid sequence (SEQ ID NO:2142) derived from the coding sequence of SEQ ID NO:2141 shown in FIG. 2141.

FIG. 2143 shows a nucleotide sequence (SEQ ID NO:2143) of a native sequence PRO9891 cDNA, wherein SEQ ID NO:2143 is a clone designated herein as “DNA328933”.

FIG. 2144 shows the amino acid sequence (SEQ ID NO:2144) derived from the coding sequence of SEQ ID NO:2143 shown in FIG. 2143.

FIG. 2145A-B shows a nucleotide sequence (SEQ ID NO:2145) of a native sequence PRO4330 cDNA, wherein SEQ ID NO:2145 is a clone designated herein as “DNA328454”.

FIG. 2146 shows the amino acid sequence (SEQ ID NO:2146) derived from the coding sequence of SEQ ID NO:2145 shown in FIG. 2145.

FIG. 2147 shows a nucleotide sequence (SEQ ID NO:2147) of a native sequence PRO34297 cDNA, wherein SEQ ID NO:2147 is a clone designated herein as “DNA217255”.

FIG. 2148 shows the amino acid sequence (SEQ ID NO:2148) derived from the coding sequence of SEQ ID NO:2147 shown in FIG. 2147.

FIG. 2149 shows a nucleotide sequence (SEQ ID NO:2149) of a native sequence PRO85096 cDNA, wherein SEQ ID NO:2149 is a clone designated herein as “DNA329551”.

FIG. 2150 shows the amino acid sequence (SEQ ID NO:2150) derived from the coding sequence of SEQ ID NO:2149 shown in FIG. 2149.

FIG. 2151 shows a nucleotide sequence (SEQ ID NO:2151) of a native sequence PRO85097 cDNA, wherein SEQ ID NO:2151 is a clone designated herein as “DNA329552”.

FIG. 2152 shows the amino acid sequence (SEQ ID NO:2152) derived from the coding sequence of SEQ ID NO:2151 shown in FIG. 2151.

FIG. 2153 shows a nucleotide sequence (SEQ ID NO:2153) of a native sequence PRO38313 cDNA, wherein SEQ ID NO:2153 is a clone designated herein as “DNA329553”.

FIG. 2154 shows the amino acid sequence (SEQ ID NO:2154) derived from the coding sequence of SEQ ID NO:2153 shown in FIG. 2153.

FIG. 2155 shows a nucleotide sequence (SEQ ID NO:2155) of a native sequence PRO85098 cDNA, wherein SEQ ID NO:2155 is a clone designated herein as “DNA329554”.

FIG. 2156 shows the amino acid sequence (SEQ ID NO:2156) derived from the coding sequence of SEQ ID NO:2155 shown in FIG. 2155.

FIG. 2157 shows a nucleotide sequence (SEQ ID NO:2157) of a native sequence PRO85099 cDNA, wherein SEQ ID NO:2157 is a clone designated herein as “DNA329555”.

FIG. 2158 shows the amino acid sequence (SEQ ID NO:2158) derived from the coding sequence of SEQ ID NO:2157 shown in FIG. 2157.

FIG. 2159 shows a nucleotide sequence (SEQ ID NO:2159) of a native sequence PRO12916 cDNA, wherein SEQ ID NO:2159 is a clone designated herein as “DNA151893”.

FIG. 2160 shows the amino acid sequence (SEQ ID NO:2160) derived from the coding sequence of SEQ ID NO:2159 shown in FIG. 2159.

FIG. 2161A-B shows a nucleotide sequence (SEQ ID NO:2161) of a native sequence cDNA, wherein SEQ ID NO:2161 is a clone designated herein as “DNA329556”.

FIG. 2162 shows a nucleotide sequence (SEQ ID NO:2162) of a native sequence PRO7250 cDNA, wherein SEQ ID NO:2162 is a clone designated herein as “DNA329557”.

FIG. 2163 shows the amino acid sequence (SEQ ID NO:2163) derived from the coding sequence of SEQ ID NO:2162 shown in FIG. 2162.

FIG. 2164 shows a nucleotide sequence (SEQ ID NO:2164) of a native sequence PRO38486 cDNA, wherein SEQ ID NO:2164 is a clone designated herein as “DNA228023”.

FIG. 2165 shows the amino acid sequence (SEQ ID NO:2165) derived from the coding sequence of SEQ ID NO:2164 shown in FIG. 2164.

FIG. 2166A-B shows a nucleotide sequence (SEQ ID NO:2166) of a native sequence PRO85100 cDNA, wherein SEQ ID NO:2166 is a clone designated herein as “DNA329558”.

FIG. 2167 shows the amino acid sequence (SEQ ID NO:2167) derived from the coding sequence of SEQ ID NO:2166 shown in FIG. 2166.

FIG. 2168 shows a nucleotide sequence (SEQ ID NO:2168) of a native sequence cDNA, wherein SEQ ID NO:2168 is a clone designated herein as “DNA150552”.

FIG. 2169 shows a nucleotide sequence (SEQ ID NO:2169) of a native sequence PRO80622 cDNA, wherein SEQ ID NO:2169 is a clone designated herein as “DNA323879”.

FIG. 2170 shows the amino acid sequence (SEQ ID NO:2170) derived from the coding sequence of SEQ ID NO:2169 shown in FIG. 2169.

FIG. 2171A-B shows a nucleotide sequence (SEQ ID NO:2171) of a native sequence PRO12843 cDNA, wherein SEQ ID NO:2171 is a clone designated herein as “DNA151027”.

FIG. 2172 shows the amino acid sequence (SEQ ID NO:2172) derived from the coding sequence of SEQ ID NO:2171 shown in FIG. 2171.

FIG. 2173 shows a nucleotide sequence (SEQ ID NO:2173) of a native sequence PRO85101 cDNA, wherein SEQ ID NO:2173 is a clone designated herein as “DNA329559”.

FIG. 2174 shows the amino acid sequence (SEQ ID NO:2174) derived from the coding sequence of SEQ ID NO:2173 shown in FIG. 2173.

FIG. 2175A-B shows a nucleotide sequence (SEQ ID NO:2175) of a native sequence PRO85102 cDNA, wherein SEQ ID NO:2175 is a clone designated herein as “DNA329560”.

FIG. 2176 shows the amino acid sequence (SEQ ID NO:2176) derived from the coding sequence of SEQ ID NO:2175 shown in FIG. 2175.

FIG. 2177 shows a nucleotide sequence (SEQ ID NO:2177) of a native sequence PRO85103 cDNA, wherein SEQ ID NO:2177 is a clone designated herein as “DNA329561”.

FIG. 2178 shows the amino acid sequence (SEQ ID NO:2178) derived from the coding sequence of SEQ ID NO:2177 shown in FIG. 2177.

FIG. 2179 shows a nucleotide sequence (SEQ ID NO:2179) of a native sequence PRO85104 cDNA, wherein SEQ ID NO:2179 is a clone designated herein as “DNA329562”.

FIG. 2180 shows the amino acid sequence (SEQ ID NO:2180) derived from the coding sequence of SEQ ID NO:2179 shown in FIG. 2179.

FIG. 2181 shows a nucleotide sequence (SEQ ID NO:2181) of a native sequence PRO84160 cDNA, wherein SEQ ID NO:2181 is a clone designated herein as “DNA328284”.

FIG. 2182 shows the amino acid sequence (SEQ ID NO:2182) derived from the coding sequence of SEQ ID NO:2181 shown in FIG. 2181.

FIG. 2183 shows a nucleotide sequence (SEQ ID NO:2183) of a native sequence PRO81947 cDNA, wherein SEQ ID NO:2183 is a clone designated herein as “DNA325421”.

FIG. 2184 shows the amino acid sequence (SEQ ID NO:2184) derived from the coding sequence of SEQ ID NO:2183 shown in FIG. 2183.

FIG. 2185A-B shows a nucleotide sequence (SEQ ID NO:2185) of a native sequence PRO1920 cDNA, wherein SEQ ID NO:2185 is a clone designated herein as “DNA329563”.

FIG. 2186 shows the amino acid sequence (SEQ ID NO:2186) derived from the coding sequence of SEQ ID NO:2185 shown in FIG. 2185.

FIG. 2187 shows a nucleotide sequence (SEQ ID NO:2187) of a native sequence PRO85105 cDNA, wherein SEQ ID NO:2187 is a clone designated herein as “DNA329564”.

FIG. 2188 shows the amino acid sequence (SEQ ID NO:2188) derived from the coding sequence of SEQ ID NO:2187 shown in FIG. 2187.

FIG. 2189 shows a nucleotide sequence (SEQ ID NO:2189) of a native sequence cDNA, wherein SEQ ID NO:2189 is a clone designated herein as “DNA196002”.

FIG. 2190A-B shows a nucleotide sequence (SEQ ID NO:2190) of a native sequence PRO84476 cDNA, wherein SEQ ID NO:2190 is a clone designated herein as “DNA328720”.

FIG. 2191 shows the amino acid sequence (SEQ ID NO:2191) derived from the coding sequence of SEQ ID NO:2190 shown in FIG. 2190.

FIG. 2192 shows a nucleotide sequence (SEQ ID NO:2192) of a native sequence PRO23880 cDNA, wherein SEQ ID NO:2192 is a clone designated herein as “DNA194522”.

FIG. 2193 shows the amino acid sequence (SEQ ID NO:2193) derived from the coding sequence of SEQ ID NO:2192 shown in FIG. 2192.

FIG. 2194 shows a nucleotide sequence (SEQ ID NO:2194) of a native sequence PRO85106 cDNA, wherein SEQ ID NO:2194 is a clone designated herein as “DNA329565”.

FIG. 2195 shows the amino acid sequence (SEQ ID NO:2195) derived from the coding sequence of SEQ ID NO:2194 shown in FIG. 2194.

FIG. 2196 shows a nucleotide sequence (SEQ ID NO:2196) of a native sequence PRO85107 cDNA, wherein SEQ ID NO:2196 is a clone designated herein as “DNA329566”.

FIG. 2197 shows the amino acid sequence (SEQ ID NO:2197) derived from the coding sequence of SEQ ID NO:2196 shown in FIG. 2196.

FIG. 2198 shows a nucleotide sequence (SEQ ID NO:2198) of a native sequence PRO69876 cDNA, wherein SEQ ID NO:2198 is a clone designated herein as “DNA328288”.

FIG. 2199 shows the amino acid sequence (SEQ ID NO:2199) derived from the coding sequence of SEQ ID NO:2198 shown in FIG. 2198.

FIG. 2200 shows a nucleotide sequence (SEQ ID NO:2200) of a native sequence PRO33775 cDNA, wherein SEQ ID NO:2200 is a clone designated herein as “DNA210241”.

FIG. 2201 shows the amino acid sequence (SEQ ID NO:2201) derived from the coding sequence of SEQ ID NO:2200 shown in FIG. 2200.

FIG. 2202 shows a nucleotide sequence (SEQ ID NO:2202) of a native sequence cDNA, wherein SEQ ID NO:2202 is a clone designated herein as “DNA33782”.

FIG. 2203 shows a nucleotide sequence (SEQ ID NO:2203) of a native sequence PRO85109 cDNA, wherein SEQ ID NO:2203 is a clone designated herein as “DNA329568”.

FIG. 2204 shows the amino acid sequence (SEQ ID NO:2204) derived from the coding sequence of SEQ ID NO:2203 shown in FIG. 2203.

FIG. 2205 shows a nucleotide sequence (SEQ ID NO:2205) of a native sequence cDNA, wherein SEQ ID NO:2205 is a clone designated herein as “DNA196162”.

FIG. 2206A-B shows a nucleotide sequence (SEQ ID NO:2206) of a native sequence PRO85110 cDNA, wherein SEQ ID NO:2206 is a clone designated herein as “DNA329569”.

FIG. 2207 shows the amino acid sequence (SEQ ID NO:2207) derived from the coding sequence of SEQ ID NO:2206 shown in FIG. 2206.

FIG. 2208 shows a nucleotide sequence (SEQ ID NO:2208) of a native sequence PRO7367 cDNA, wherein SEQ ID NO:2208 is a clone designated herein as “DNA90840”.

FIG. 2209 shows the amino acid sequence (SEQ ID NO:2209) derived from the coding sequence of SEQ ID NO:2208 shown in FIG. 2208.

FIG. 2210 shows a nucleotide sequence (SEQ ID NO:2210) of a native sequence cDNA, wherein SEQ ID NO:2210 is a clone designated herein as “DNA329570”.

FIG. 2211 shows a nucleotide sequence (SEQ ID NO:2211) of a native sequence PRO2391 cDNA, wherein SEQ ID NO:2211 is a clone designated herein as “DNA88516”.

FIG. 2212 shows the amino acid sequence (SEQ ID NO:2212) derived from the coding sequence of SEQ ID NO:2211 shown in FIG. 2211.

FIG. 2213 shows a nucleotide sequence (SEQ ID NO:2213) of a native sequence PRO50208 cDNA, wherein SEQ ID NO:2213 is a clone designated herein as “DNA255127”.

FIG. 2214 shows the amino acid sequence (SEQ ID NO:2214) derived from the coding sequence of SEQ ID NO:2213 shown in FIG. 2213.

FIG. 2215 shows a nucleotide sequence (SEQ ID NO:2215) of a native sequence PRO50791 cDNA, wherein SEQ ID NO:2215 is a clone designated herein as “DNA255734”.

FIG. 2216 shows the amino acid sequence (SEQ ID NO:2216) derived from the coding sequence of SEQ ID NO:2215 shown in FIG. 2215.

FIG. 2217 shows a nucleotide sequence (SEQ ID NO:2217) of a native sequence PRO51662 cDNA, wherein SEQ ID NO:2217 is a clone designated herein as “DNA329571”.

FIG. 2218 shows the amino acid sequence (SEQ ID NO:2218) derived from the coding sequence of SEQ ID NO:2217 shown in FIG. 2217.

FIG. 2219 shows a nucleotide sequence (SEQ ID NO:2219) of a native sequence PRO51611 cDNA, wherein SEQ ID NO:2219 is a clone designated herein as “DNA256608”.

FIG. 2220 shows the amino acid sequence (SEQ ID NO:2220) derived from the coding sequence of SEQ ID NO:2219 shown in FIG. 2219.

FIG. 2221 shows a nucleotide sequence (SEQ ID NO:2221) of a native sequence PRO85111 cDNA, wherein SEQ ID NO:2221 is a clone designated herein as “DNA329572”.

FIG. 2222 shows the amino acid sequence (SEQ ID NO:2222) derived from the coding sequence of SEQ ID NO:2221 shown in FIG. 2221.

FIG. 2223 shows a nucleotide sequence (SEQ ID NO:2223) of a native sequence PRO50241 cDNA, wherein SEQ ID NO:2223 is a clone designated herein as “DNA255161”.

FIG. 2224 shows the amino acid sequence (SEQ ID NO:2224) derived from the coding sequence of SEQ ID NO:2223 shown in FIG. 2223.

FIG. 2225 shows a nucleotide sequence (SEQ ID NO:2225) of a native sequence PRO62346 cDNA, wherein SEQ ID NO:2225 is a clone designated herein as “DNA274437”.

FIG. 2226 shows the amino acid sequence (SEQ ID NO:2226) derived from the coding sequence of SEQ ID NO:2225 shown in FIG. 2225.

FIG. 2227 shows a nucleotide sequence (SEQ ID NO:2227) of a native sequence PRO49615 cDNA, wherein SEQ ID NO:2227 is a clone designated herein as “DNA254508”.

FIG. 2228 shows the amino acid sequence (SEQ ID NO:2228) derived from the coding sequence of SEQ ID NO:2227 shown in FIG. 2227.

FIG. 2229 shows a nucleotide sequence (SEQ ID NO:2229) of a native sequence PRO2386 cDNA, wherein SEQ ID NO:2229 is a clone designated herein as “DNA885 11”.

FIG. 2230 shows the amino acid sequence (SEQ ID NO:2230) derived from the coding sequence of SEQ ID NO:2229 shown in FIG. 2229.

FIG. 2231 shows a nucleotide sequence (SEQ ID NO:2231) of a native sequence PRO51556 cDNA, wherein SEQ ID NO:2231 is a clone designated herein as “DNA256521”.

FIG. 2232 shows the amino acid sequence (SEQ ID NO:2232) derived from the coding sequence of SEQ ID NO:2231 shown in FIG. 2231.

FIG. 2233 shows a nucleotide sequence (SEQ ID NO:2233) of a native sequence PRO51565 cDNA, wherein SEQ ID NO:2233 is a clone designated herein as “DNA256533”.

FIG. 2234 shows the amino acid sequence (SEQ ID NO:2234) derived from the coding sequence of SEQ ID NO:2233 shown in FIG. 2233.

FIG. 2235 shows a nucleotide sequence (SEQ ID NO:2235) of a native sequence PRO85112 cDNA, wherein SEQ ID NO:2235 is a clone designated herein as “DNA329573”.

FIG. 2236 shows the amino acid sequence (SEQ ID NO:2236) derived from the coding sequence of SEQ ID NO:2235 shown in FIG. 2235.

FIG. 2237 shows a nucleotide sequence (SEQ ID NO:2237) of a native sequence PRO59409 cDNA, wherein SEQ ID NO:2237 is a clone designated herein as “DNA271085”.

FIG. 2238 shows the amino acid sequence (SEQ ID NO:2238) derived from the coding sequence of SEQ ID NO:2237 shown in FIG. 2237.

FIG. 2239 shows a nucleotide sequence (SEQ ID NO:2239) of a native sequence PRO85113 cDNA, wherein SEQ ID NO:2239 is a clone designated herein as “DNA329574”.

FIG. 2240 shows the amino acid sequence (SEQ ID NO:) derived from the coding sequence of SEQ ID NO: shown in Figure

FIG. 2241 shows a nucleotide sequence (SEQ ID NO:2241) of a native sequence PRO61403 cDNA, wherein SEQ ID NO:2241 is a clone designated herein as “DNA329575”.

FIG. 2242 shows the amino acid sequence (SEQ ID NO:2242) derived from the coding sequence of SEQ ID NO:2241 shown in FIG. 2241.

FIG. 2243 shows a nucleotide sequence (SEQ ID NO:2243) of a native sequence PRO64127 cDNA, wherein SEQ ID NO:2243 is a clone designated herein as “DNA329576”.

FIG. 2244 shows the amino acid sequence (SEQ ID NO:2244) derived from the coding sequence of SEQ ID NO:2243 shown in FIG. 2243.

FIG. 2245 shows a nucleotide sequence (SEQ ID NO:2245) of a native sequence PRO61623 cDNA, wherein SEQ ID NO:2245 is a clone designated herein as “DNA273653”.

FIG. 2246 shows the amino acid sequence (SEQ ID NO:2246) derived from the coding sequence of SEQ ID NO:2245 shown in FIG. 2245.

FIG. 2247 shows a nucleotide sequence (SEQ ID NO:2247) of a native sequence PRO50191 cDNA, wherein SEQ ID NO:2247 is a clone designated herein as “DNA329577”.

FIG. 2248 shows the amino acid sequence (SEQ ID NO:2248) derived from the coding sequence of SEQ ID NO:2247 shown in FIG. 2247.

FIG. 2249 shows a nucleotide sequence (SEQ ID NO:2249) of a native sequence PRO51820 cDNA, wherein SEQ ID NO:2249 is a clone designated herein as “DNA329578”.

FIG. 2250 shows the amino acid sequence (SEQ ID NO:2250) derived from the coding sequence of SEQ ID NO:2249 shown in FIG. 2249.

FIG. 2251 shows a nucleotide sequence (SEQ ID NO:2251) of a native sequence PRO69609 cDNA, wherein SEQ ID NO:2251 is a clone designated herein as “DNA329579”.

FIG. 2252 shows the amino acid sequence (SEQ ID NO:2252) derived from the coding sequence of SEQ ID NO:2251 shown in FIG. 2251.

FIG. 2253 shows a nucleotide sequence (SEQ ID NO:2253) of a native sequence PRO57311 cDNA, wherein SEQ ID NO:2253 is a clone designated herein as “DNA327927”.

FIG. 2254 shows the amino acid sequence (SEQ ID NO:2254) derived from the coding sequence of SEQ ID NO:2253 shown in FIG. 2253.

FIG. 2255 shows a nucleotide sequence (SEQ ID NO:2255) of a native sequence PRO58197 cDNA, wherein SEQ ID NO:2255 is a clone designated herein as “DNA269791”.

FIG. 2256 shows the amino acid sequence (SEQ ID NO:2256) derived from the coding sequence of SEQ ID NO:2255 shown in FIG. 2255.

FIG. 2257 shows a nucleotide sequence (SEQ ID NO:2257) of a native sequence PRO85114 cDNA, wherein SEQ ID NO:2257 is a clone designated herein as “DNA329580”.

FIG. 2258 shows the amino acid sequence (SEQ ID NO:2258) derived from the coding sequence of SEQ ID NO:2257 shown in FIG. 2257.

FIG. 2259 shows a nucleotide sequence (SEQ ID NO:2259) of a native sequence PRO85115 cDNA, wherein SEQ ID NO:2259 is a clone designated herein as “DNA329581”.

FIG. 2260 shows the amino acid sequence (SEQ ID NO:2260) derived from the coding sequence of SEQ ID NO:2259 shown in FIG. 2259.

FIG. 2261 shows a nucleotide sequence (SEQ ID NO:2261) of a native sequence PRO51602 cDNA, wherein SEQ ID NO:2261 is a clone designated herein as “DNA256578”.

FIG. 2262 shows the amino acid sequence (SEQ ID NO:2262) derived from the coding sequence of SEQ ID NO:2261 shown in FIG. 2261.

FIG. 2263 shows a nucleotide sequence (SEQ ID NO:2263) of a native sequence PRO49368 cDNA, wherein SEQ ID NO:2263 is a clone designated herein as “DNA254256”.

FIG. 2264 shows the amino acid sequence (SEQ ID NO:2264) derived from the coding sequence of SEQ ID NO:2263 shown in FIG. 2263.

FIG. 2265 shows a nucleotide sequence (SEQ ID NO:2265) of a native sequence PRO50216 cDNA, wherein SEQ ID NO:2265 is a clone designated herein as “DNA255135”.

FIG. 2266 shows the amino acid sequence (SEQ ID NO:2266) derived from the coding sequence of SEQ ID NO:2265 shown in FIG. 2265.

FIG. 2267 shows a nucleotide sequence (SEQ ID NO:2267) of a native sequence PRO50365 cDNA, wherein SEQ ID NO:2267 is a clone designated herein as “DNA255292”.

FIG. 2268 shows the amino acid sequence (SEQ ID NO:2268) derived from the coding sequence of SEQ ID NO:2267 shown in FIG. 2267.

FIG. 2269 shows a nucleotide sequence (SEQ ID NO:2269) of a native sequence PRO85116 cDNA, wherein SEQ ID NO:2269 is a clone designated herein as “DNA329582”.

FIG. 2270 shows the amino acid sequence (SEQ ID NO:2270) derived from the coding sequence of SEQ ID NO:2269 shown in FIG. 2269.

FIG. 2271 shows a nucleotide sequence (SEQ ID NO:2271) of a native sequence PRO50544 cDNA, wherein SEQ ID NO:2271 is a clone designated herein as “DNA255477”.

FIG. 2272 shows the amino acid sequence (SEQ ID NO:2272) derived from the coding sequence of SEQ ID NO:2271 shown in FIG. 2271.

FIG. 2273 shows a nucleotide sequence (SEQ ID NO:2273) of a native sequence PRO85117 cDNA, wherein SEQ ID NO:2273 is a clone designated herein as “DNA329583”.

FIG. 2274 shows the amino acid sequence (SEQ ID NO:2274) derived from the coding sequence of SEQ ID NO:2273 shown in FIG. 2273.

FIG. 2275 shows a nucleotide sequence (SEQ ID NO:2275) of a native sequence PRO85118 cDNA, wherein SEQ ID NO:2275 is a clone designated herein as “DNA329584”.

FIG. 2276 shows the amino acid sequence (SEQ ID NO:2276) derived from the coding sequence of SEQ ID NO:2275 shown in FIG. 2275.

FIG. 2277 shows a nucleotide sequence (SEQ ID NO:2277) of a native sequence PRO59911 cDNA, wherein SEQ ID NO:2277 is a clone designated herein as “DNA271624”.

FIG. 2278 shows the amino acid sequence (SEQ ID NO:2278) derived from the coding sequence of SEQ ID NO:2277 shown in FIG. 2277.

FIG. 2279 shows a nucleotide sequence (SEQ ID NO:2279) of a native sequence PRO69503 cDNA, wherein SEQ ID NO:2279 is a clone designated herein as “DNA287224”.

FIG. 2280 shows the amino acid sequence (SEQ ID NO:2280) derived from the coding sequence of SEQ ID NO:2279 shown in FIG. 2279.

FIG. 2281 shows a nucleotide sequence (SEQ ID NO:2281) of a native sequence PRO59278 cDNA, wherein SEQ ID NO:2281 is a clone designated herein as “DNA270947”.

FIG. 2282 shows the amino acid sequence (SEQ ID NO:2282) derived from the coding sequence of SEQ ID NO:2281 shown in FIG. 2281.

FIG. 2283 shows a nucleotide sequence (SEQ ID NO:2283) of a native sequence PRO85119 cDNA, wherein SEQ ID NO:2283 is a clone designated herein as “DNA329585”.

FIG. 2284 shows the amino acid sequence (SEQ ID NO:2284) derived from the coding sequence of SEQ ID NO:2283 shown in FIG. 2283.

FIG. 2285 shows a nucleotide sequence (SEQ ID NO:2285) of a native sequence PRO85120 cDNA, wherein SEQ ID NO:2285 is a clone designated herein as “DNA329586”.

FIG. 2286 shows the amino acid sequence (SEQ ID NO:2286) derived from the coding sequence of SEQ ID NO:2285 shown in FIG. 2285.

FIG. 2287 shows a nucleotide sequence (SEQ ID NO:2287) of a native sequence PRO51584 cDNA, wherein SEQ ID NO:2287 is a clone designated herein as “DNA256553”.

FIG. 2288 shows the amino acid sequence (SEQ ID NO:2288) derived from the coding sequence of SEQ ID NO:2287 shown in FIG. 2287.

FIG. 2289 shows a nucleotide sequence (SEQ ID NO:2289) of a native sequence PRO61504 cDNA, wherein SEQ ID NO:2289 is a clone designated herein as “DNA273523”.

FIG. 2290 shows the amino acid sequence (SEQ ID NO:2290) derived from the coding sequence of SEQ ID NO:2289 shown in FIG. 2289.

FIG. 2291 shows a nucleotide sequence (SEQ ID NO:2291) of a native sequence PRO58541 cDNA, wherein SEQ ID NO:2291 is a clone designated herein as “DNA270152”.

FIG. 2292 shows the amino acid sequence (SEQ ID NO:2292) derived from the coding sequence of SEQ ID NO:2291 shown in FIG. 2291.

FIG. 2293 shows a nucleotide sequence (SEQ ID NO:2293) of a native sequence PRO85121 cDNA, wherein SEQ ID NO:2293 is a clone designated herein as “DNA329587”.

FIG. 2294 shows the amino acid sequence (SEQ ID NO:2294) derived from the coding sequence of SEQ ID NO:2293 shown in FIG. 2293

FIG. 2295 shows a nucleotide sequence (SEQ ID NO:2295) of a native sequence PRO50682 cDNA, wherein SEQ ID NO:2295 is a clone designated herein as “DNA255619”.

FIG. 2296 shows the amino acid sequence (SEQ ID NO:2296) derived from the coding sequence of SEQ ID NO:2295 shown in FIG. 2295.

FIG. 2297 shows a nucleotide sequence (SEQ ID NO:2297) of a native sequence PRO85122 cDNA, wherein SEQ ID NO:2297 is a clone designated herein as “DNA329588”.

FIG. 2298 shows the amino acid sequence (SEQ ID NO:2298) derived from the coding sequence of SEQ ID NO:2297 shown in FIG. 2297

FIG. 2299 shows a nucleotide sequence (SEQ ID NO:2299) of a native sequence cDNA, wherein SEQ ID NO:2299 is a clone designated herein as “DNA2561 10”.

FIG. 2300 shows a nucleotide sequence (SEQ ID NO:2300) of a native sequence PRO85123 cDNA, wherein SEQ ID NO:2300 is a clone designated herein as “DNA329589”.

FIG. 2301 shows the amino acid sequence (SEQ ID NO:2301) derived from the coding sequence of SEQ ID NO:2300 shown in FIG. 2300.

FIG. 2302A-B shows a nucleotide sequence (SEQ ID NO:2302) of a native sequence PRO84706 cDNA, wherein SEQ ID NO:2302 is a clone designated herein as “DNA329039”.

FIG. 2303 shows the amino acid sequence (SEQ ID NO:2303) derived from the coding sequence of SEQ ID NO:2302 shown in FIG. 2302.

FIG. 2304 shows a nucleotide sequence (SEQ ID NO:2304) of a native sequence PRO57996 cDNA, wherein SEQ ID NO:2304 is a clone designated herein as “DNA328509”.

FIG. 2305 shows the amino acid sequence (SEQ ID NO:2305) derived from the coding sequence of SEQ ID NO:2304 shown in FIG. 2304.

FIG. 2306 shows a nucleotide sequence (SEQ ID NO:2306) of a native sequence PRO50614 cDNA, wherein SEQ ID NO:2306 is a clone designated herein as “DNA329590”.

FIG. 2307 shows the amino acid sequence (SEQ ID NO:2307) derived from the coding sequence of SEQ ID NO:2306 shown in FIG. 2306.

FIG. 2308 shows a nucleotide sequence (SEQ ID NO:2308) of a native sequence cDNA, wherein SEQ ID NO:2308 is a clone designated herein as “DNA329591”.

FIG. 2309 shows a nucleotide sequence (SEQ ID NO:2309) of a native sequence PRO85124 cDNA, wherein SEQ ID NO:2309 is a clone designated herein as “DNA329592”.

FIG. 2310 shows the amino acid sequence (SEQ ID NO:2310) derived from the coding sequence of SEQ ID NO:2309 shown in FIG. 2309.

FIG. 2311 shows a nucleotide sequence (SEQ ID NO:2311) of a native sequence PRO51777 cDNA, wherein SEQ ID NO:2311 is a clone designated herein as “DNA256846”.

FIG. 2312 shows the amino acid sequence (SEQ ID NO:2312) derived from the coding sequence of SEQ ID NO:2311 shown in FIG. 2311.

FIG. 2313 shows a nucleotide sequence (SEQ ID NO:2313) of a native sequence PRO85125 cDNA, wherein SEQ ID NO:2313 is a clone designated herein as “DNA329593”.

FIG. 2314 shows the amino acid sequence (SEQ ID NO:2314) derived from the coding sequence of SEQ ID NO:2313 shown in FIG. 2313.

FIG. 2315 shows a nucleotide sequence (SEQ ID NO:2315) of a native sequence PRO50261 cDNA, wherein SEQ ID NO:2315 is a clone designated herein as “DNA255181”.

FIG. 2316 shows the amino acid sequence (SEQ ID NO:2316) derived from the coding sequence of SEQ ID NO:2315 shown in FIG. 2315.

FIG. 2317 shows a nucleotide sequence (SEQ ID NO:2317) of a native sequence cDNA, wherein SEQ ID NO:2317 is a clone designated herein as “DNA329594”.

FIG. 2318 shows a nucleotide sequence (SEQ ID NO:2318) of a native sequence PRO85127 cDNA, wherein SEQ ID NO:2318 is a clone designated herein as “DNA329595”.

FIG. 2319 shows the amino acid sequence (SEQ ID NO:2319) derived from the coding sequence of SEQ ID NO:2318 shown in FIG. 2318.

FIG. 2320A-B shows a nucleotide sequence (SEQ ID NO:2320) of a native sequence PRO85128 cDNA, wherein SEQ ID NO:2320 is a clone designated herein as “DNA329596”.

FIG. 2321 shows the amino acid sequence (SEQ ID NO:2321) derived from the coding sequence of SEQ ID NO:2320 shown in FIG. 2320.

FIG. 2322 shows a nucleotide sequence (SEQ ID NO:2322) of a native sequence PRO85129 cDNA, wherein SEQ ID NO:2322 is a clone designated herein as “DNA329597”.

FIG. 2323 shows the amino acid sequence (SEQ ID NO:2323) derived from the coding sequence of SEQ ID NO:2322 shown in FIG. 2322.

FIG. 2324 shows a nucleotide sequence (SEQ ID NO:2324) of a native sequence PRO23253 cDNA, wherein SEQ ID NO:2324 is a clone designated herein as “DNA329078”.

FIG. 2325 shows the amino acid sequence (SEQ ID NO:2325) derived from the coding sequence of SEQ ID NO:2324 shown in FIG. 2324.

FIG. 2326 shows a nucleotide sequence (SEQ ID NO:2326) of a native sequence PRO85130 cDNA, wherein SEQ ID NO:2326 is a clone designated herein as “DNA329598”.

FIG. 2327 shows the amino acid sequence (SEQ ID NO:2327) derived from the coding sequence of SEQ ID NO:2326 shown in FIG. 2326.

FIG. 2328 shows a nucleotide sequence (SEQ ID NO:2328) of a native sequence PRO60456 cDNA, wherein SEQ ID NO:2328 is a clone designated herein as “DNA272191”.

FIG. 2329 shows the amino acid sequence (SEQ ID NO:2329) derived from the coding sequence of SEQ ID NO:2328 shown in FIG. 2328.

FIG. 2330A-B shows a nucleotide sequence (SEQ ID NO:2330) of a native sequence PRO85131 cDNA, wherein SEQ ID NO:2330 is a clone designated herein as “DNA329599”.

FIG. 2331 shows the amino acid sequence (SEQ ID NO:2331) derived from the coding sequence of SEQ ID NO:2230 shown in FIG. 2230.

FIG. 2332 shows a nucleotide sequence (SEQ ID NO:2332) of a native sequence PRO81261 cDNA, wherein SEQ ID NO:2332 is a clone designated herein as “DNA324612”.

FIG. 2333 shows the amino acid sequence (SEQ ID NO:2333) derived from the coding sequence of SEQ ID NO:2332 shown in FIG. 2332

FIG. 2334 shows a nucleotide sequence (SEQ ID NO:2334) of a native sequence PRO59570 cDNA, wherein SEQ ID NO:2334 is a clone designated herein as “DNA329600”.

FIG. 2335 shows the amino acid sequence (SEQ ID NO:2335) derived from the coding sequence of SEQ ID NO:2334 shown in FIG. 2334.

FIG. 2336A-B shows a nucleotide sequence (SEQ ID NO:2336) of a native sequence PRO50357 cDNA, wherein SEQ ID NO:2336 is a clone designated herein as “DNA255281”.

FIG. 2337 shows the amino acid sequence (SEQ ID NO:2337) derived from the coding sequence of SEQ ID NO:2336 shown in FIG. 2336.

FIG. 2338 shows a nucleotide sequence (SEQ ID NO:2338) of a native sequence PRO58933 cDNA, wherein SEQ ID NO:2338 is a clone designated herein as “DNA270558”.

FIG. 2339 shows the amino acid sequence (SEQ ID NO:2339) derived from the coding sequence of SEQ ID NO:2338 shown in FIG. 2338.

FIG. 2340 shows a nucleotide sequence (SEQ ID NO:2340) of a native sequence PRO82373 cDNA, wherein SEQ ID NO:2340 is a clone designated herein as “DNA325920”.

FIG. 2341 shows the amino acid sequence (SEQ ID NO:2341) derived from the coding sequence of SEQ ID NO:2340 shown in FIG. 2340.

FIG. 2342 shows a nucleotide sequence (SEQ ID NO:2342) of a native sequence PRO58993 cDNA, wherein SEQ ID NO:2342 is a clone designated herein as “DNA324690”.

FIG. 2343 shows the amino acid sequence (SEQ ID NO:2343) derived from the coding sequence of SEQ ID NO:2342 shown in FIG. 2342.

FIG. 2344 shows a nucleotide sequence (SEQ ID NO:2344) of a native sequence PRO57930 cDNA, wherein SEQ ID NO:2344 is a clone designated herein as “DNA269514”.

FIG. 2345 shows the amino acid sequence (SEQ ID NO:2345) derived from the coding sequence of SEQ ID NO:2344 shown in FIG. 2344.

FIG. 2346 shows a nucleotide sequence (SEQ ID NO:2346) of a native sequence PRO85132 cDNA, wherein SEQ ID NO:2346 is a clone designated herein as “DNA329601”.

FIG. 2347 shows the amino acid sequence (SEQ ID NO:2347) derived from the coding sequence of SEQ ID NO:2346 shown in FIG. 2346.

FIG. 2348 shows a nucleotide sequence (SEQ ID NO:2348) of a native sequence PRO49288 cDNA, wherein SEQ ID NO:2348 is a clone designated herein as “DNA254175”.

FIG. 2349 shows the amino acid sequence (SEQ ID NO:2349) derived from the coding sequence of SEQ ID NO:2348 shown in FIG. 2348.

FIG. 2350 shows a nucleotide sequence (SEQ ID NO:2350) of a native sequence PRO58175 cDNA, wherein SEQ ID NO:2350 is a clone designated herein as “DNA269766”.

FIG. 2351 shows the amino acid sequence (SEQ ID NO:2351) derived from the coding sequence of SEQ ID NO:2350 shown in FIG. 2350.

FIG. 2352 shows a nucleotide sequence (SEQ ID NO:2352) of a native sequence PRO85133 cDNA, wherein SEQ ID NO:2352 is a clone designated herein as “DNA329602”.

FIG. 2353 shows the amino acid sequence (SEQ ID NO:2353) derived from the coding sequence of SEQ ID NO:2352 shown in FIG. 2352.

FIG. 2354A-B shows a nucleotide sequence (SEQ ID NO:2354) of a native sequence PRO59246 cDNA, wherein SEQ ID NO:2354 is a clone designated herein as “DNA329603”.

FIG. 2355 shows the amino acid sequence (SEQ ID NO:2355) derived from the coding sequence of SEQ ID NO:2354 shown in FIG. 2354.

FIG. 2356 shows a nucleotide sequence (SEQ ID NO:2356) of a native sequence PRO85134 cDNA, wherein SEQ ID NO:2356 is a clone designated herein as “DNA329604”.

FIG. 2357 shows the amino acid sequence (SEQ ID NO:2357) derived from the coding sequence of SEQ ID NO:2356 shown in FIG. 2356.

FIG. 2358 shows a nucleotide sequence (SEQ ID NO:2358) of a native sequence PRO85135 cDNA, wherein SEQ ID NO:2358 is a clone designated herein as “DNA329605”.

FIG. 2359 shows the amino acid sequence (SEQ ID NO:2359) derived from the coding sequence of SEQ ID NO:2358 shown in FIG. 2358.

FIG. 2360 shows a nucleotide sequence (SEQ ID NO:2360) of a native sequence PRO58219 cDNA, wherein SEQ ID NO:2360 is a clone designated herein as “DNA269816”.

FIG. 2361 shows the amino acid sequence (SEQ ID NO:2361) derived from the coding sequence of SEQ ID NO:2360 shown in FIG. 2360.

FIG. 2362 shows a nucleotide sequence (SEQ ID NO:2362) of a native sequence PRO58754 cDNA, wherein SEQ ID NO:2362 is a clone designated herein as “DNA270369”.

FIG. 2363 shows the amino acid sequence (SEQ ID NO:2363) derived from the coding sequence of SEQ ID NO:2362 shown in FIG. 2362.

FIG. 2364 shows a nucleotide sequence (SEQ ID NO:2364) of a native sequence PRO85136 cDNA, wherein SEQ ID NO:2364 is a clone designated herein as “DNA329606”.

FIG. 2365 shows the amino acid sequence (SEQ ID NO:2365) derived from the coding sequence of SEQ ID NO:2364 shown in FIG. 2364.

FIG. 2366 shows a nucleotide sequence (SEQ ID NO:2366) of a native sequence PRO81893 cDNA, wherein SEQ ID NO:2366 is a clone designated herein as “DNA325355”.

FIG. 2367 shows the amino acid sequence (SEQ ID NO:2367) derived from the coding sequence of SEQ ID NO:2366 shown in FIG. 2366.

FIG. 2368 shows a nucleotide sequence (SEQ ID NO:2368) of a native sequence PRO85137 cDNA, wherein SEQ ID NO:2368 is a clone designated herein as “DNA329607”.

FIG. 2369 shows the amino acid sequence (SEQ ID NO:2369) derived from the coding sequence of SEQ ID NO:2368 shown in FIG. 2368.

FIG. 2370 shows a nucleotide sequence (SEQ ID NO:2370) of a native sequence PRO70699 cDNA, wherein SEQ ID NO:2370 is a clone designated herein as “DNA293243”.

FIG. 2371 shows the amino acid sequence (SEQ ID NO:2371) derived from the coding sequence of SEQ ID NO:2370 shown in FIG. 2370.

FIG. 2372 shows a nucleotide sequence (SEQ ID NO:2372) of a native sequence PRO2388 cDNA, wherein SEQ ID NO:2372 is a clone designated herein as “DNA88513”.

FIG. 2373 shows the amino acid sequence (SEQ ID NO:2373) derived from the coding sequence of SEQ ID NO:2372 shown in FIG. 2372.

FIG. 2374 shows a nucleotide sequence (SEQ ID NO:2374) of a native sequence PRO85138 cDNA, wherein SEQ ID NO:2374 is a clone designated herein as “DNA329608”.

FIG. 2375 shows the amino acid sequence (SEQ ID NO:2375) derived from the coding sequence of SEQ ID NO:2374 shown in FIG. 2374.

FIG. 2376A-B shows a nucleotide sequence (SEQ ID NO:2376) of a native sequence cDNA, wherein SEQ ID NO:2376 is a clone designated herein as “DNA329051”.

FIG. 2377 shows a nucleotide sequence (SEQ ID NO:2377) of a native sequence PRO85139 cDNA, wherein SEQ ID NO:2377 is a clone designated herein as “DNA329609”.

FIG. 2378 shows the amino acid sequence (SEQ ID NO:2378) derived from the coding sequence of SEQ ID NO:2378 shown in Figure

FIG. 2379 shows a nucleotide sequence (SEQ ID NO:2379) of a native sequence PRO84183 cDNA, wherein SEQ ID NO:2379 is a clone designated herein as “DNA328315”.

FIG. 2380 shows the amino acid sequence (SEQ ID NO:2380) derived from the coding sequence of SEQ ID NO:2379 shown in FIG. 2379.

FIG. 2381 shows a nucleotide sequence (SEQ ID NO:2381) of a native sequence cDNA, herein SEQ ID NO:2381 is a clone designated herein as “DNA329610”.

FIG. 2382 shows a nucleotide sequence (SEQ ID NO:2382) of a native sequence PRO85141 cDNA, wherein SEQ ID NO:2382 is a clone designated herein as “DNA329611”.

FIG. 2383 shows the amino acid sequence (SEQ ID NO:2383) derived from the coding sequence of SEQ ID NO:2382 shown in FIG. 2382.

FIG. 2384 shows a nucleotide sequence (SEQ ID NO:2384) of a native sequence cDNA, wherein SEQ ID NO:2384 is a clone designated herein as “DNA256198”.

FIG. 2385 shows a nucleotide sequence (SEQ ID NO:2385) of a native sequence PRO85142 cDNA, wherein SEQ ID NO:2385 is a clone designated herein as “DNA329612”.

FIG. 2386 shows the amino acid sequence (SEQ ID NO:2386) derived from the coding sequence of SEQ ID NO:2385 shown in FIG. 2385.

FIG. 2387 shows a nucleotide sequence (SEQ ID NO:2387) of a native sequence PRO85143 cDNA, wherein SEQ ID NO:2387 is a clone designated herein as “DNA329613”.

FIG. 2388 shows the amino acid sequence (SEQ ID NO:2388) derived from the coding sequence of SEQ ID NO:2387 shown in FIG. 2387.

FIG. 2389 shows a nucleotide sequence (SEQ ID NO:2389) of a native sequence cDNA, wherein SEQ ID NO:2389 is a clone designated herein as “DNA329614”.

FIG. 2390 shows a nucleotide sequence (SEQ ID NO:2390) of a native sequence PRO50379 cDNA, wherein SEQ ID NO:2390 is a clone designated herein as “DNA255306”.

FIG. 2391 shows the amino acid sequence (SEQ ID NO:2391) derived from the coding sequence of SEQ ID NO:2390 shown in FIG. 2390.

FIG. 2392 shows a nucleotide sequence (SEQ ID NO:2392) of a native sequence PRO70559 cDNA, wherein SEQ ID NO:2392 is a clone designated herein as “DNA290812”.

FIG. 2393 shows the amino acid sequence (SEQ ID NO:2393) derived from the coding sequence of SEQ ID NO:2392 shown in FIG. 2392.

FIG. 2394 shows a nucleotide sequence (SEQ ID NO:2394) of a native sequence cDNA, wherein SEQ ID NO:2394 is a clone designated herein as “DNA256085”.

FIG. 2395A-B shows a nucleotide sequence (SEQ ID NO:2395) of a native sequence PRO84584 cDNA, wherein SEQ ID NO:2395 is a clone designated herein as “DNA328853”.

FIG. 2396 shows the amino acid sequence (SEQ ID NO:2396) derived from the coding sequence of SEQ ID NO:2395 shown in FIG. 2395.

FIG. 2397 shows a nucleotide sequence (SEQ ID NO:2397) of a native sequence PRO85144 cDNA, wherein SEQ ID NO:2397 is a clone designated herein as “DNA329615”.

FIG. 2398 shows the amino acid sequence (SEQ ID NO:2398) derived from the coding sequence of SEQ ID NO:2397 shown in FIG. 2397.

FIG. 2399 shows a nucleotide sequence (SEQ ID NO:2399) of a native sequence cDNA, wherein SEQ ID NO:2399 is a clone designated herein as “DNA329616”.

FIG. 2400 shows a nucleotide sequence (SEQ ID NO:2400) of a native sequence cDNA, wherein SEQ ID NO:2400 is a clone designated herein as “DNA257960”.

FIG. 2401 shows a nucleotide sequence (SEQ ID NO:2401) of a native sequence PRO85146 cDNA, wherein SEQ ID NO:2401 is a clone designated herein as “DNA329617”.

FIG. 2402 shows the amino acid sequence (SEQ ID NO:2402) derived from the coding sequence of SEQ ID NO:2401 shown in FIG. 2401.

FIG. 2403 shows a nucleotide sequence (SEQ ID NO:2403) of a native sequence PRO52682 cDNA, wherein SEQ ID NO:2403 is a clone designated herein as “DNA258747”.

FIG. 2404 shows the amino acid sequence (SEQ ID NO:2404) derived from the coding sequence of SEQ ID NO:2403 shown in FIG. 2403.

FIG. 2405 shows a nucleotide sequence (SEQ ID NO:2405) of a native sequence cDNA, wherein SEQ ID NO:2405 is a clone designated herein as “DNA258793”.

FIG. 2406 shows a nucleotide sequence (SEQ ID NO:2406) of a native sequence cDNA, wherein SEQ ID NO:2406 is a clone designated herein as “DNA258683”.

FIG. 2407 shows a nucleotide sequence (SEQ ID NO:2407) of a native sequence PRO85147 cDNA, wherein SEQ ID NO:2407 is a clone designated herein as “DNA329618”.

FIG. 2408 shows the amino acid sequence (SEQ ID NO:2408) derived from the coding sequence of SEQ ID NO:2407 shown in FIG. 2407.

FIG. 2409 shows a nucleotide sequence (SEQ ID NO:2409) of a native sequence PRO85148 cDNA, wherein SEQ ID NO:2409 is a clone designated herein as “DNA329619”.

FIG. 2410 shows the amino acid sequence (SEQ ID NO:2410) derived from the coding sequence of SEQ ID NO:2409 shown in FIG. 2409.

FIG. 2411 shows a nucleotide sequence (SEQ ID NO:2411) of a native sequence PRO85149 cDNA, wherein SEQ ID NO:2411 is a clone designated herein as “DNA329620”.

FIG. 2412 shows the amino acid sequence (SEQ ID NO:2412) derived from the coding sequence of SEQ ID NO:2411 shown in FIG. 2411.

FIG. 2413 shows a nucleotide sequence (SEQ ID NO:2413) of a native sequence cDNA, wherein SEQ ID NO:2413 is a clone designated herein as “DNA258763”.

FIG. 2414 shows a nucleotide sequence (SEQ ID NO:2414) of a native sequence PRO85150 cDNA, wherein SEQ ID NO:2414 is a clone designated herein as “DNA329621”.

FIG. 2415 shows the amino acid sequence (SEQ ID NO:2415) derived from the coding sequence of SEQ ID NO:2414 shown in FIG. 2414.

FIG. 2416 shows a nucleotide sequence (SEQ ID NO:2416) of a native sequence cDNA, wherein SEQ ID NO:2416 is a clone designated herein as “DNA259435”.

FIG. 2417 shows a nucleotide sequence (SEQ ID NO:2417) of a native sequence PRO53966 cDNA, wherein SEQ ID NO:2417 is a clone designated herein as “DNA260036”.

FIG. 2418 shows the amino acid sequence (SEQ ID NO:2418) derived from the coding sequence of SEQ ID NO:2417 shown in FIG. 2417.

FIG. 2419 shows a nucleotide sequence (SEQ ID NO:2419) of a native sequence PRO85151 cDNA, wherein SEQ ID NO:2419 is a clone designated herein as “DNA329622”.

FIG. 2420 shows the amino acid sequence (SEQ ID NO:2420) derived from the coding sequence of SEQ ID NO:2419 shown in FIG. 2419.

FIG. 2421 shows a nucleotide sequence (SEQ ID NO:2421) of a native sequence PRO85152 cDNA, wherein SEQ ID NO:2421 is a clone designated herein as “DNA329623”.

FIG. 2422 shows the amino acid sequence (SEQ ID NO:2422) derived from the coding sequence of SEQ ID NO:2421 shown in FIG. 2421.

FIG. 2423 shows a nucleotide sequence (SEQ ID NO:2423) of a native sequence PRO85153 cDNA, wherein SEQ ID NO:2423 is a clone designated herein as “DNA329624”.

FIG. 2424 shows the amino acid sequence (SEQ ID NO:2424) derived from the coding sequence of SEQ ID NO:2423 shown in FIG. 2423.

FIG. 2425 shows a nucleotide sequence (SEQ ID NO:2425) of a native sequence PRO85154 cDNA, wherein SEQ ID NO:2425 is a clone designated herein as “DNA329625”.

FIG. 2426 shows the amino acid sequence (SEQ ID NO:2426) derived from the coding sequence of SEQ ID NO:2424 shown in FIG. 2425.

FIG. 2427 shows a nucleotide sequence (SEQ ID NO:2427) of a native sequence cDNA, wherein SEQ ID NO:2427 is a clone designated herein as “DNA258637”.

FIG. 2428 shows a nucleotide sequence (SEQ ID NO:2428) of a native sequence cDNA, wherein SEQ ID NO:2428 is a clone designated herein as “DNA262810”.

FIG. 2429 shows a nucleotide sequence (SEQ ID NO:2429) of a native sequence PRO51901 cDNA, wherein SEQ ID NO:2429 is a clone designated herein as “DNA257309”.

FIG. 2430 shows the amino acid sequence (SEQ ID NO:2430) derived from the coding sequence of SEQ ID NO:2429 shown in FIG. 2429.

FIG. 2431 shows a nucleotide sequence (SEQ ID NO:2431) of a native sequence PRO85155 cDNA, wherein SEQ ID NO:2431 is a clone designated herein as “DNA329626”.

FIG. 2432 shows the amino acid sequence (SEQ ID NO:2432) derived from the coding sequence of SEQ ID NO:2431 shown in FIG. 2431.

FIG. 2433 shows a nucleotide sequence (SEQ ID NO:2433) of a native sequence PRO85156 cDNA, wherein SEQ ID NO:2433 is a clone designated herein as “DNA329627”.

FIG. 2434 shows the amino acid sequence (SEQ ID NO:2434) derived from the coding sequence of SEQ ID NO:2433 shown in FIG. 2433.

FIG. 2435 shows a nucleotide sequence (SEQ ID NO:2435) of a native sequence PRO53004 cDNA, wherein SEQ ID NO:2435 is a clone designated herein as “DNA259071”.

FIG. 2436 shows the amino acid sequence (SEQ ID NO:2436) derived from the coding sequence of SEQ ID NO:2435 shown in FIG. 2435.

FIG. 2437 shows a nucleotide sequence (SEQ ID NO:2437) of a native sequence PRO85157 cDNA, wherein SEQ ID NO:2437 is a clone designated herein as “DNA329628”.

FIG. 2438 shows the amino acid sequence (SEQ ID NO:2438) derived from the coding sequence of SEQ ID NO:2437 shown in FIG. 2437.

FIG. 2439 shows a nucleotide sequence (SEQ ID NO:2439) of a native sequence PRO85158 cDNA, wherein SEQ ID NO:2439 is a clone designated herein as “DNA329629”.

FIG. 2440 shows the amino acid sequence (SEQ ID NO:2440) derived from the coding sequence of SEQ ID NO:2439 shown in FIG. 2439.

FIG. 2441 shows a nucleotide sequence (SEQ ID NO:2441) of a native sequence PRO52822 cDNA, wherein SEQ ID NO:2441 is a clone designated herein as “DNA258889”.

FIG. 2442 shows the amino acid sequence (SEQ ID NO:2442) derived from the coding sequence of SEQ ID NO:2441 shown in FIG. 2441.

### DETAILED DESCRIPTION OF THE PREFERRED EMBODIMENTS

I. Definitions

The terms “PRO polypeptide” and “PRO” as used herein and when immediately followed by a numerical designation refer to various polypeptides, wherein the complete designation (i.e., PRO/number) refers to specific polypeptide sequences as described herein. The terms “PRO/number polypeptide” and “PRO/number” wherein the term “number” is provided as an actual numerical designation as used herein encompass native sequence polypeptides and polypeptide variants (which are further defined herein). The PRO polypeptides described herein may be isolated from a variety of sources, such as from human tissue types or from another source, or prepared by recombinant or synthetic methods. The term “PRO polypeptide” refers to each individual PRO/number polypeptide disclosed herein. All disclosures in this specification which refer to the “PRO polypeptide” refer to each of the polypeptides individually as well as jointly. For example, descriptions of the preparation of, purification of, derivation of, formation of antibodies to or against, administration of, compositions containing, treatment of a disease with, etc., pertain to each polypeptide of the invention individually. The term “PRO polypeptide” also includes variants of the PRO/number polypeptides disclosed herein.

A “native sequence PRO polypeptide” comprises a polypeptide having the same amino acid sequence as the corresponding PRO polypeptide derived from nature. Such native sequence PRO polypeptides can be isolated from nature or can be produced by recombinant or synthetic means. The term “native sequence PRO polypeptide” specifically encompasses naturally-occurring truncated or secreted forms of the specific PRO polypeptide (e.g., an extracellular domain sequence), naturally-occurring variant forms (e.g., alternatively spliced forms) and naturally-occurring allelic variants of the polypeptide. In various embodiments of the invention, the native sequence PRO polypeptides disclosed herein are mature or full-length native sequence polypeptides comprising the full-length amino acids sequences shown in the accompanying figures. Start and stop codons are shown in bold font and underlined in the figures. However, while the PRO polypeptide disclosed in the accompanying figures are shown to begin with methionine residues designated herein as amino acid position 1 in the figures, it is conceivable and possible that other methionine residues located either upstream or downstream from the amino acid position 1 in the figures may be employed as the starting amino acid residue for the PRO polypeptides.

The PRO polypeptide “extracellular domain” or “ECD” refers to a form of the PRO polypeptide which is essentially free of the transmembrane and cytoplasmic domains. Ordinarily, a PRO polypeptide ECD will have less than 1% of such transmembrane and/or cytoplasmic domains and preferably, will have less than 0.5% of such domains. It will be understood that any transmembrane domains identified for the PRO polypeptides of the present invention are identified pursuant to criteria routinely employed in the art for identifying that type of hydrophobic domain. The exact boundaries of a transmembrane domain may vary but most likely by no more than about 5 amino acids at either end of the domain as initially identified herein. Optionally, therefore, an extracellular domain of a PRO polypeptide may contain from about 5 or fewer amino acids on either side of the transmembrane domain/extracellular domain boundary as identified in the Examples or specification and such polypeptides, with or without the associated signal peptide, and nucleic acid encoding them, are contemplated by the present invention.

The approximate location of the “signal peptides” of the various PRO polypeptides disclosed herein are shown in the present specification and/or the accompanying figures. It is noted, however, that the C-terminal boundary of a signal peptide may vary, but most likely by no more than about 5 amino acids on either side of the signal peptide C-terminal boundary as initially identified herein, wherein the C-terminal boundary of the signal peptide may be identified pursuant to criteria routinely employed in the art for identifying that type of amino acid sequence element (e.g., Nielsen et al., Prot. Eng. 10: 1-6 (1997) and von Heinje et al., Nucl. Acids. Res. 14:4683-4690 (1986)). Moreover, it is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

“PRO polypeptide variant” means an active PRO polypeptide as defined above or below having at least about 80% amino acid sequence identity with a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Such PRO polypeptide variants include, for instance, PRO polypeptides wherein one or more amino acid residues are added, or deleted, at the N- or C-terminus of the full-length native amino acid sequence. Ordinarily, a PRO polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length native sequence PRO polypeptide sequence as disclosed herein, a PRO polypeptide sequence lacking the signal peptide as disclosed herein, an extracellular domain of a PRO polypeptide, with or without the signal peptide, as disclosed herein or any other specifically defined fragment of a full-length PRO polypeptide sequence as disclosed herein. Ordinarily, PRO variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, alternatively at least about 200 amino acids in length, alternatively at least about 300 amino acids in length, or more.

“Percent (%) amino acid sequence identity” with respect to the PRO polypeptide sequences identified herein is defined as the percentage of amino acid residues in a candidate sequence that are identical with the amino acid residues in the specific PRO polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, Tables 2 and 3 demonstrate how to calculate the % amino acid sequence identity of the amino acid sequence designated “Comparison Protein” to the amino acid sequence designated “PRO”, wherein “PRO” represents the amino acid sequence of a hypothetical PRO polypeptide of interest, “Comparison Protein” represents the amino acid sequence of a polypeptide against which the “PRO” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the PRO polypeptide of interest having a sequence derived from the native PRO polypeptide and the comparison amino acid sequence of interest (i.e., the sequence against which the PRO polypeptide of interest is being compared which may be a PRO variant polypeptide) as determined by WU-BLAST-2 by (b) the total number of amino acid residues of the PRO polypeptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the PRO polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PRO variant polynucleotide” or “PRO variant nucleic acid sequence” means a nucleic acid molecule which encodes an active PRO polypeptide as defined below and which has at least about 80% nucleic acid sequence identity with a nucleotide acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal peptide, as disclosed herein or any other fragment of a full-length PRO polypeptide sequence as disclosed herein. Ordinarily, a PRO variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about [symbol] nucleic acid sequence identity, alternatively at least about 93% nucleic acid sequence identity, alternatively at least about 94% nucleic acid sequence identity, alternatively at least about 95% nucleic acid [symbol] sequence identity, alternatively at least about 96% nucleic acid sequence identity, alternatively at least about [symbol] nucleic acid sequence identity, alternatively at least about 98% nucleic acid sequence identity and [symbol] alternatively at least about 99% nucleic acid sequence identity with a nucleic acid sequence encoding a full-length native sequence PRO polypeptide sequence as disclosed herein, a full-length native sequence PRO polypeptide sequence lacking the signal peptide as disclosed herein, an extracellular domain of a PRO polypeptide, with or without the signal sequence, as disclosed herein or any other fragment of a full-length PRO polypeptide sequence as disclosed herein. Variants do not encompass the native nucleotide sequence.

Ordinarily, PRO variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to PRO-encoding nucleic acid sequences identified herein is defined as the percentage of nucleotides in a candidate sequence that are identical with the nucleotides in the PRO nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “PRO-DNA”, wherein “PRO-DNA” represents a hypothetical PRO-encoding nucleic acid sequence of interest, “Comparison DNA” represents the nucleotide sequence of a nucleic acid molecule against which the “PRO-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the PRO polypeptide-encoding nucleic acid molecule of interest having a sequence derived from the native sequence PRO polypeptide-encoding nucleic acid and the comparison nucleic acid molecule of interest (i.e., the sequence against which the PRO polypeptide-encoding nucleic acid molecule of interest is being compared which may be a variant PRO polynucleotide) as determined by WU-BLAST-2 by (b) the total number of nucleotides of the PRO polypeptide-encoding nucleic acid molecule of interest. For example, in the statement “an isolated nucleic acid molecule comprising a nucleic acid sequence A which has or having at least 80% nucleic acid sequence identity to the nucleic acid sequence B”, the nucleic acid sequence A is the comparison nucleic acid molecule of interest and the nucleic acid sequence B is the nucleic acid sequence of the PRO polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z
where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PRO variant polynucleotides are nucleic acid molecules that encode an active PRO polypeptide and which are capable of hybridizing, preferably under stringent hybridization and wash conditions, to nucleotide sequences encoding a full-length PRO polypeptide as disclosed herein. PRO variant polypeptides may be those that are encoded by a PRO variant polynucleotide.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PRO polypeptide natural environment will not be present. Ordinarily, however, isolated polypeptide will be prepared by at least one purification step.

An “isolated” PRO polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PRO monoclonal antibodies (including agonist, antagonist, and neutralizing antibodies), anti-PRO antibody compositions with polyepitopic specificity, single chain anti-PRO antibodies, and fragments of anti-PRO antibodies (see below). The term “monoclonal antibody” as used herein refers to an antibody obtained from a population of substantially homogeneous antibodies, i.e., the individual antibodies comprising the population are identical except for possible naturally-occurring mutations that may be present in minor amounts.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PRO polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of a PRO polypeptide which retain a biological and/or an immunological activity of native or naturally-occurring PRO, wherein “biological” activity refers to a biological function (either inhibitory or stimulatory) caused by a native or naturally-occurring PRO other than the ability to induce the production of an antibody against an antigenic epitope possessed by a native or naturally-occurring PRO and an “immunological” activity refers to the ability to induce the production of an antibody against an antigenic epitope possessed by a native or naturally-occurring PRO.

The term “antagonist” is used in the broadest sense, and includes any molecule that partially or fully blocks, inhibits, or neutralizes a biological activity of a native PRO polypeptide disclosed herein. In a similar manner, the term “agonist” is used in the broadest sense and includes any molecule that mimics a biological activity of a native PRO polypeptide disclosed herein. Suitable agonist or antagonist molecules specifically include agonist or antagonist antibodies or antibody fragments, fragments or amino acid sequence variants of native PRO polypeptides, peptides, antisense oligonucleotides, small organic molecules, etc. Methods for identifying agonists or antagonists of a PRO polypeptide may comprise contacting a PRO polypeptide with a candidate agonist or antagonist molecule and measuring a detectable change in one or more biological activities normally associated with the PRO polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, [symbol] immunoglobulins can be assigned to different classes. There are five major classes of immunoglobulins: [symbol] IgD, IgE, IgG, and IgM, and several of these may be further divided into subclasses (isotypes), e.g., [symbol] IgG1, IgG2, IgG3, IgG4, IgA, and IgA2.

“Single-chain Fv” or “sFv” antibody fragments comprise the V_{H} and V_{L} domains of antibody, wherein these domains are present in a single polypeptide chain. Preferably, the Fv polypeptide further [symbol] comprises a polypeptide linker between the V_{H} and V_{L} domains which enables the sFv to form the desired [symbol] ture for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The word “label” when used herein refers to a detectable compound or composition which is conjugated directly or indirectly to the antibody so as to generate a “labeled” antibody. The label may be detectable by itself (e.g. radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PRO polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to a morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

The term “effective amount” is a concentration or amount of a PRO polypeptide and/or agonist/antagonist which results in achieving a particular stated purpose. An “effective amount” of a PRO polypeptide or agonist or antagonist thereof may be determined empirically. Furthermore, a “therapeutically effective amount” is a concentration or amount of a PRO polypeptide and/or agonist/antagonist which is effective for achieving a stated therapeutic effect. This amount may also be determined empirically.

The term “cytotoxic agent” as used herein refers to a substance that inhibits or prevents the function of cells and/or causes destruction of cells. The term is intended to include radioactive isotopes (e.g., I¹³¹, I¹²⁵, Y⁹⁰ and Re¹⁸⁶), chemotherapeutic agents, and toxins such as enzymatically active toxins of bacterial, fungal, plant or animal origin, or fragments thereof.

A “chemotherapeutic agent” is a chemical compound useful in the treatment of cancer. Examples of chemotherapeutic agents include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (Taxotere, Rhône-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

A “growth inhibitory agent” when used herein refers to a compound or composition which inhibits growth of a cell, especially cancer cell overexpressing any of the genes identified herein, either in vitro or in vivo. Thus, the growth inhibitory agent is one which significantly reduces the percentage of cells overexpressing such genes in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (WB Saunders: Philadelphia, 1995), especially p. 13.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

As used herein, the term “inflammatory cells” designates cells that enhance the inflammatory response such as mononuclear cells, eosinophils, macrophages, and polymorphonuclear neutrophils (PMN).

TABLE 2

| | | |
|---|---|---|
| PRO | XXXXXXXXXXXXXXX | (Length = 15 amino acids) |
| Comparison | XXXXXYYYYYYY | (Length = 12 amino acids) |
| Protein | | |

% amino acid sequence identity =(the number of identically matching amino acid residues between the two polypeptide sequences as determined by ALIGN-2) divided by (the total number of amino acid residues of the PRO polypeptide) = 5 divided by 15 = 33.3%

TABLE 3

| | | |
|---|---|---|
| PRO | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the PRO polypeptide) = 5 divided by 10 = 50%

TABLE 4

| | | |
|---|---|---|
| PRO-DNA | NNNNNNNNNNNNNN | (Length = 14 nucleotides) |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 6 divided by 14 = 42.9%

TABLE 5

| | | |
|---|---|---|
| PRO-DNA | NNNNNNNNNNNN | (Length = 12 nucleotides) |
| Comparison | NNNNLLLVV | (Length = 9 nucleotides) |
| DNA | | |

% nucleic acid sequence identity = (the number of identically matching nucleotides between the two nucleic acid sequences as determined by ALIGN-2) divided by (the total number of nucleotides of the PRO-DNA nucleic acid sequence) = 4 divided by 12 = 33.3%

II. Compositions and Methods of the Invention

A. Full-Length PRO Polypeptides

The present invention provides newly identified and isolated nucleotide sequences encoding polypeptides referred to in the present application as PRO polypeptides. In particular, cDNAs encoding various PRO polypeptides have been identified and isolated, as disclosed in further detail in the Examples below. However, for sake of simplicity, in the present specification the protein encoded by the full length native nucleic acid molecules disclosed herein as well as all further native homologues and variants included in the foregoing definition of PRO, will be referred to as “PRO/number”, regardless of their origin or mode of preparation.

As disclosed in the Examples below, various cDNA clones have been disclosed. The predicted amino acid sequence can be determined from the nucleotide sequence using routine skill. For the PRO polypeptides and encoding nucleic acids described herein, Applicants have identified what is believed to be the reading frame best identifiable with the sequence information available at the time.

B. PRO Polypeptide Variants

In addition to the full-length native sequence PRO polypeptides described herein, it is contemplated that PRO variants can be prepared. PRO variants can be prepared by introducing appropriate nucleotide changes into the PRO DNA, and/or by synthesis of the desired PRO polypeptide. Those skilled in the art will appreciate that amino acid changes may alter post-translational processes of the PRO, such as changing the number or position of glycosylation sites or altering the membrane anchoring characteristics.

Variations in the native full-length sequence PRO or in various domains of the PRO described herein, can be made, for example, using any of the techniques and guidelines for conservative and non-conservative mutations set forth, for instance, in U.S. Pat. No. 5,364,934. Variations may be a substitution, deletion or insertion of one or more codons encoding the PRO that results in a change in the amino acid sequence of the PRO as compared with the native sequence PRO. Optionally, the variation is by substitution of at least one amino acid with any other amino acid in one or more of the domains of the PRO. Guidance in determining which amino acid residue may be inserted, substituted or deleted without adversely affecting the desired activity may be found by comparing the sequence of the PRO with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

PRO polypeptide fragments are provided herein. Such fragments may be truncated at the N-terminus or C-terminus, or may lack internal residues, for example, when compared with a full length native protein. Certain fragments lack amino acid residues that are not essential for a desired biological activity of the PRO polypeptide.

PRO fragments may be prepared by any of a number of conventional techniques. Desired peptide fragments may be chemically synthesized. An alternative approach involves generating PRO fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PRO polypeptide fragments share at least one biological and/or immunological activity with the native PRO polypeptide disclosed herein.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened.

TABLE 6

| Original | Exemplary | Preferred |
|---|---|---|
| Residue | Substitutions | Substitutions |
| Ala (A) | val; leu; ile | val |
| Arg (R) | lys; gin; asn | lys |
| Asn (N) | gln; his; lys; arg | gln |
| [symbol] (D) | glu | glu |
| [symbol] (C) | ser | ser |
| [symbol] (Q) | asn | asn |
| [symbol] (E) | asp | asp |
| Gly (G) | pro; ala | ala |
| [symbol] (H) | asn; gln; lys; arg | arg |
| [symbol] | leu; val; met; ala; phe; | leu |
| | norleucine | |
| [symbol] (L) | norleucine; ile; val; | ile |
| | met; ala; phe | |
| [symbol] (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of the PRO polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gln, his, lys, arg;

(5) residues that influence chain orientation: gly, pro; and

(6) aromatic: trp, tyr, phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the PRO variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-=bon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

C. Modifications of PRO

Covalent modifications of PRO are included within the scope of this invention. One type of covalent modification includes reacting targeted amino acid residues of a PRO polypeptide with an organic derivatizing agent that is capable of reacting with selected side chains or the N- or C-terminal residues of the PRO. Derivatization with bifunctional agents is useful, for instance, for crosslinking PRO to a water-insoluble support matrix or surface for use in the method for purifying anti-PRO antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the PRO polypeptide included within the scope of this invention comprises altering the native glycosylation pattern of the polypeptide. “Altering the native glycosylation pattern” is intended for purposes herein to mean deleting one or more carbohydrate moieties found in native sequence PRO (either by removing the underlying glycosylation site or by deleting the glycosylation by chemical and/or enzymatic means), and/or adding one or more glycosylation sites that are not present in the native sequence PRO. In addition, the phrase includes qualitative changes in the glycosylation of the native proteins, involving a change in the nature and proportions of the various carbohydrate moieties present.

Addition of glycosylation sites to the PRO polypeptide may be accomplished by altering the amino acid sequence. The alteration may be made, for example, by the addition of, or substitution by, one or more serine or threonine residues to the native sequence PRO (for O-linked glycosylation sites). The PRO amino acid sequence may optionally be altered through changes at the DNA level, particularly by mutating the DNA encoding the PRO polypeptide at preselected bases such that codons are generated that will translate into the desired amino acids.

Another means of increasing the number of carbohydrate moieties on the PRO polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PRO polypeptide may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PRO comprises linking the PRO polypeptide to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337.

The PRO of the present invention may also be modified in a way to form a chimeric molecule comprising PRO fused to another, heterologous polypeptide or amino acid sequence.

In one embodiment, such a chimeric molecule comprises a fusion of the PRO with a tag polypeptide which provides an epitope to which an anti-tag antibody can selectively bind. The epitope tag is generally placed at the amino- or carboxyl-terminus of the PRO. The presence of such epitope-tagged forms of the PRO can be detected using an antibody against the tag polypeptide. Also, provision of the epitope tag enables the PRO to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an alpha-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the PRO with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PRO polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

D. Preparation of PRO

The description below relates primarily to production of PRO by culturing cells transformed or transfected with a vector containing PRO nucleic acid. It is, of course, contemplated that alternative methods, which are well known in the art, may be employed to prepare PRO. For instance, the PRO sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of the PRO may be chemically synthesized separately and combined using chemical or enzymatic methods to produce the full-length PRO.

1. Isolation of DNA Encoding PRO

DNA encoding PRO may be obtained from a cDNA library prepared from tissue believed to possess the PRO mRNA and to express it at a detectable level. Accordingly, human PRO DNA can be conveniently obtained from a cDNA library prepared from human tissue, such as described in the Examples. The PRO-encoding gene may also be obtained from a genomic library or by known synthetic procedures (e.g., automated nucleic acid synthesis).

Libraries can be screened with probes (such as antibodies to the PRO or oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). An alternative means to isolate the gene encoding PRO is to use PCR methodology [Sambrook et al., supra; Dieffenbach et al., PCR Primer: A Laboratory Manual (Cold Spring Harbor Laboratory Press, 1995)].

The Examples below describe techniques for screening a cDNA library. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for PRO production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for PRO-encoding vectors. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulanis (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated PRO are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); Chinese hamster ovary cells/-DHFR (CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

3. Selection and Use of a Replicable Vector

The nucleic acid (e.g., cDNA or genomic DNA) encoding PRO may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The PRO may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the PRO-encoding DNA that is inserted into the vector. The [symbol] sequence may be a prokaryotic signal sequence selected, for example, from the group of the alkaline [symbol] phatase, penicillinase, lpp, or heat-stable enterotoxin II leaders. For yeast secretion the signal sequence [symbol] be, e.g., the yeast invertase leader, alpha factor leader (including Saccharomyces and Kluyveromyces α [symbol] -leaders, the latter described in U.S. Pat. No. 5,010,182), or acid phosphatase leader, the C. albicans [symbol] amylase leader (EP 362,179 published 4 Apr. 1990), or the signal described in WO 90/13646 [symbol] ed 15 Nov. 1990. In mammalian cell expression, mammalian signal sequences may be used to [symbol] secretion of the protein, such as signal sequences from secreted polypeptides of the same or related [symbol] species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the PRO-encoding nucleic acid, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the PRO-encoding nucleic acid sequence to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding PRO.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistry, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

PRO transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the PRO by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the PRO coding sequence, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding PRO.

Still other methods, vectors, and host cells suitable for adaptation to the synthesis of PRO in recombinant vertebrate cell culture are described in Gething et al., Nature, 293:620-625 (1981); Mantei et al., Nature, 281:40-46 (1979); EP 117,060; and EP 117,058.

4. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA. 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence PRO polypeptide or against a synthetic peptide based on the DNA sequences provided herein or against exogenous sequence fused to PRO DNA and encoding a specific antibody epitope.

5. Purification of Polypeptide

Forms of PRO may be recovered from culture medium or from host cell lysates. If membrane-bound, it can be released from the membrane using a suitable detergent solution (e.g. Triton-X 100) or by enzymatic cleavage. Cells employed in expression of PRO can be disrupted by various physical or chemical means, such as freeze-thaw cycling, sonication, mechanical disruption, or cell lysing agents.

It may be desired to purify PRO from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PRO. Various methods of protein purification may be employed and such methods are known in the art and described for example in Deutscher, Methods in Enzymology 182 (1990); Scopes, Protein Purification: Principles and Practice, Springer-Verlag, New York (1982). The purification step(s) selected will depend, for example, on the nature of the production process used and the particular PRO produced.

E. Tissue Distribution

The location of tissues expressing the PRO can be identified by determining mRNA expression in various human tissues. The location of such genes provides information about which tissues are most likely to be affected by the stimulating and inhibiting activities of the PRO polypeptides. The location of a gene in a specific tissue also provides sample tissue for the activity blocking assays discussed below.

As noted before, gene expression in various tissues may be measured by conventional Southern blotting, Northern blotting to quantitate the transcription of mRNA (Thomas, Proc. Natl. Acad. Sci. USA, 77:5201-5205 [1980]), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes.

Gene expression in various tissues, alternatively, may be measured by immunological methods, such as immunohistochemical staining of tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies may be prepared against a native sequence of a PRO polypeptide or against a synthetic peptide based on the DNA sequences encoding the PRO polypeptide or against an exogenous sequence fused to a DNA encoding a PRO polypeptide and encoding a specific antibody epitope. General techniques for generating antibodies, and special protocols for Northern blotting and in situ hybridization are provided below.

F. Antibody Binding Studies

The activity of the PRO polypeptides can be further verified by antibody binding studies, in which the ability of anti-PRO antibodies to inhibit the effect of the PRO polypeptides, respectively, on tissue cells is tested. Exemplary antibodies include polyclonal, monoclonal, humanized, bispecific, and heteroconjugate antibodies, the preparation of which will be described hereinbelow.

Antibody binding studies may be carried out in any known assay method, such as competitive binding assays, direct and indirect sandwich assays, and immunoprecipitation assays. Zola, Monoclonal Antibodies: A Manual of Techniques, pp. 147-158 (CRC Press, Inc., 1987).

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies preferably are insolubilized before or after the competition, so that the standard and analyte that are bound to the antibodies may conveniently be separated from the standard and analyte which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the tissue sample may be fresh or frozen or may be embedded in paraffin and fixed with a preservative such as formalin, for example.

G. Cell-Based Assays

Cell-based assays and animal models for immune related diseases can be used to further understand the relationship between the genes and polypeptides identified herein and the development and pathogenesis of immune related disease.

In a different approach, cells of a cell type known to be involved in a particular immune related disease are transfected with the cDNAs described herein, and the ability of these cDNAs to stimulate or inhibit immune function is analyzed. Suitable cells can be transfected with the desired gene, and monitored for immune function activity. Such transfected cell lines can then be used to test the ability of poly- or monoclonal antibodies or antibody compositions to inhibit or stimulate immune function, for example to modulate T-cell proliferation or inflammatory cell infiltration. Cells transfected with the coding sequences of the genes identified herein can further be used to identify drug candidates for the treatment of immune related diseases.

In addition, primary cultures derived from transgenic animals (as described below) can be used in the cell-based assays herein, although stable cell lines are preferred. Techniques to derive continuous cell lines from transgenic animals are well known in the art (see, e.g., Small et al., Mol. Cell. Biol. 5: 642-648 [1985]).

One suitable cell based assay is the mixed lymphocyte reaction (MLR). Current Protocols in Immunology, unit 3.12; edited by J E Coligan, A M Kruisbeek, D H Marglies, B M Shevach, W Strober, National Institutes of Health, Published by John Wiley & Sons, Inc. In this assay, the ability of a test compound to stimulate or inhibit the proliferation of activated T cells is assayed. A suspension of responder T cells is cultured with allogeneic stimulator cells and the proliferation of T cells is measured by uptake of tritiated thymidine. This assay is a general measure of T cell reactivity. Since the majority of T cells respond to and produce IL-2 upon activation, differences in responsiveness in this assay in part reflect differences in IL-2 production by the responding cells. The MLR results can be verified by a standard lymphokine (IL-2) detection assay. Current Protocols in Immunology, above, 3.15, 6.3.

A proliferative T cell response in an MLR assay may be due to direct mitogenic properties of an assayed molecule or to external antigen induced activation. Additional verification of the T cell stimulatory activity of the PRO polypeptides can be obtained by a costimulation assay. T cell activation requires an antigen specific signal mediated through the T-cell receptor (TCR) and a costimulatory signal mediated through a second ligand binding interaction, for example, the B7 (CD80, CD86)/CD28 binding interaction. CD28 crosslinking increases lymphokine secretion by activated T cells. T cell activation has both negative and positive controls through the binding of ligands which have a negative or positive effect. CD28 and CTLA-4 are related glycoproteins in the Ig superfamily which bind to B7. CD28 binding to B7 has a positive costimulation effect of T cell activation; conversely, CTLA-4 binding to B7 has a T cell deactivating effect Chambers, C. A. and Allison, J. P., Curr. Opin. Immunol. (1997) 9:396. Schwartz, R. H., Cell (1992) 71:1065; Linsey, P. S. and Ledbetter, J. A., Annu. Rev. Immunol. (1993) 11:191; June, C. H. et al, Immunol. Today (1994) 15:321; Jenkins, M. K., Immunity (1994) 1:405. In a costimulation assay, the PRO polypeptides are assayed for T cell costimulatory or inhibitory activity.

Direct use of a stimulating compound as in the invention has been validated in experiments with 4-1BB glycoprotein, a member of the tumor necrosis factor receptor family, which binds to a ligand (4-1BBL) expressed on primed T cells and signals T cell activation and growth. Alderson, M. E. et al., J. Immunol. (1994) 24:2219.

The use of an agonist stimulating compound has also been validated experimentally. Activation of 4-1BB by treatment with an agonist anti-4-1BB antibody enhances eradication of tumors. Hellstrom, I. and Hellstrom, K. E., Crit. Rev. Immunol. (1998) 18:1. Immunoadjuvant therapy for treatment of tumors, described in more detail below, is another example of the use of the stimulating compounds of the invention.

Alternatively, an immune stimulating or enhancing effect can also be achieved by administration of a PRO which has vascular permeability enhancing properties. Enhanced vascular permeability would be beneficial to disorders which can be attenuated by local infiltration of immune cells (e.g., monocytes, eosinophils, PMNs) and inflammation.

On the other hand, PRO polypeptides, as well as other compounds of the invention, which are direct inhibitors of T cell proliferation/activation, lymphokine secretion, and/or vascular permeability can be directly used to suppress the immune response. These compounds are useful to reduce the degree of the immune response and to treat immune related diseases characterized by a hyperactive, superoptimal, or autoimmune response. This use of the compounds of the invention has been validated by the experiments described above in which CTLA-4 binding to receptor B7 deactivates T cells. The direct inhibitory compounds of the invention function in an analogous manner. The use of compound which suppress vascular permeability would be expected to reduce inflammation. Such uses would be beneficial in treating conditions associated with excessive inflammation.

Alternatively, compounds, e.g., antibodies, which bind to stimulating PRO polypeptides and block the stimulating effect of these molecules produce a net inhibitory effect and can be used to suppress the T cell mediated immune response by inhibiting T cell proliferation/activation and/or lymphokine secretion. Blocking the stimulating effect of the polypeptides suppresses the immune response of the mammal. This use has been validated in experiments using an anti-IL2 antibody. In these experiments, the antibody binds to IL2 and blocks binding of IL2 to its receptor thereby achieving a T cell inhibitory effect.

H. Animal Models

The results of the cell based in vitro assays can be further verified using in vivo animal models and assays for T-cell function. A variety of well known animal models can be used to further understand the role of the genes identified herein in the development and pathogenesis of immune related disease, and to test the efficacy of candidate therapeutic agents, including antibodies, and other antagonists of the native polypeptides, including small molecule antagonists. The in vivo nature of such models makes them predictive of responses in human patients. Animal models of immune related diseases include both non-recombinant and recombinant (transgenic) animals. Non-recombinant animal models include, for example, rodent, e.g., murine models. Such models can be generated by introducing cells into syngeneic mice using standard techniques, e.g., subcutaneous injection, tail vein injection, spleen implantation, intraperitoneal implantation, implantation under the renal capsule, etc.

Graft-versus-host disease occurs when immunocompetent cells are transplanted into immunosuppressed or tolerant patients. The donor cells recognize and respond to host antigens. The response can vary from life threatening severe inflammation to mild cases of diarrhea and weight loss. Graft-versus-host disease models provide a means of assessing T cell reactivity against MHC antigens and minor transplant antigens. A suitable procedure is described in detail in Current Protocols in Immunology, above, unit 4.3.

An animal model for skin allograft rejection is a means of testing the ability of T cells to mediate in vivo tissue destruction and a measure of their role in transplant rejection. The most common and accepted models use murine tail-skin grafts. Repeated experiments have shown that skin allograft rejection is mediated by T cells, helper T cells and killer-effector T cells, and not antibodies. Auchincloss, H. Jr. and Sachs, D. H., Fundamental Immunology, 2nd ed., W. E. Paul ed., Raven Press, NY, 1989, 889-992. A suitable procedure is described in detail in Current Protocols in Immunology, above, unit 4.4. Other transplant rejection models which can be used to test the compounds of the invention are the allogeneic heart transplant models described by Tanabe, M. et al, Transplantation (1994) 58:23 and Tinubu, S. A. et al., J. Immunol. (1994) 4330-4338.

Animal models for delayed type hypersensitivity provides an assay of cell mediated immune function as well. Delayed type hypersensitivity reactions are a T cell mediated in vivo immune response characterized by inflammation which does not reach a peak until after a period of time has elapsed after challenge with an antigen. These reactions also occur in tissue specific autoimmune diseases such as [symbol] Multiple sclerosis (MS) and experimental autoimmune encephalomyelitis (EAE, a model for MS). A [symbol] able procedure is described in detail in Current Protocols in Immunology, above, unit 4.5.

EAE is a T cell mediated autoimmune disease characterized by T cell and mononuclear cell [symbol] inflammation and subsequent demyelination of axons in the central nervous system. EAE is generally [symbol] dered to be a relevant animal model for MS in humans. Bolton, C., Multiple Sclerosis (1995) 1:143. [symbol] a acute and relapsing-remitting models have been developed. The compounds of the invention can be [symbol] for T cell stimulatory or inhibitory activity against immune mediated demyelinating disease using the [symbol] ocol described in Current Protocols in Immunology, above, units 15.1 and 15.2. See also the models for myelin disease in which oligodendrocytes or Schwann cells are grafted into the central nervous system as described in Duncan, I. D. et al, Molec. Med. Today (1997) 554-561.

Contact hypersensitivity is a simple delayed type hypersensitivity in vivo assay of cell mediated immune function. In this procedure, cutaneous exposure to exogenous haptens which gives rise to a delayed type hypersensitivity reaction which is measured and quantitated. Contact sensitivity involves an initial sensitizing phase followed by an elicitation phase. The elicitation phase occurs when the T lymphocytes encounter an antigen to which they have had previous contact. Swelling and inflammation occur, making this an excellent model of human allergic contact dermatitis. A suitable procedure is described in detail in Current Protocols in Immunology, Eds. J. E. Cologan, A. M. Kruisbeek, D. H. Margulies, E. M. Shevach and W. Strober, John Wiley & Sons, Inc., 1994, unit 4.2. See also Grabbe, S. and Schwarz, T, Immun. Today 19 (1): 3744 (1998).

An animal model for arthritis is collagen-induced arthritis. This model shares clinical, histological and immunological characteristics of human autoimmune rheumatoid arthritis and is an acceptable model for human autoimmune arthritis. Mouse and rat models are characterized by synovitis, erosion of cartilage and subchondral bone. The compounds of the invention can be tested for activity against autoimmune arthritis using the protocols described in Current Protocols in Immunology, above, units 15.5. See also the model using a monoclonal antibody to CD18 and VLA-4 integrins described in Issekutz, A. C. et al., Immunology (1996) 88:569.

A model of asthma has been described in which antigen-induced airway hyper-reactivity, pulmonary eosinophilia and inflammation are induced by sensitizing an animal with ovalbumin and then challenging the animal with the same protein delivered by aerosol. Several animal models (guinea pig, rat, non-human primate) show symptoms similar to atopic asthma in humans upon challenge with aerosol antigens. Murine models have many of the features of human asthma. Suitable procedures to test the compounds of the invention for activity and effectiveness in the treatment of asthma are described by Wolyniec, W. W. et al, Am. J. Respir. Cell Mol. Biol. (1998) 18:777 and the references cited therein.

Additionally, the compounds of the invention can be tested on animal models for psoriasis like diseases. Evidence suggests a T cell pathogenesis for psoriasis. The compounds of the invention can be tested in the scid/scid mouse model described by Schon, M. P. et al Nat. Med. (1997) 3:183, in which the mice demonstrate histopathologic skin lesions resembling psoriasis. Another suitable model is the human skid/scid mouse chimera prepared as described by Nickoloff, B. J. et al, Am. J. Path. (1995) 146:580.

Recombinant (transgenic) animal models can be engineered by introducing the coding portion of the genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (Hoppe and Wanger, U.S. Pat. No. 4,873,191); retrovirus-mediated gene transfer into germ lines (e.g., Van der Putten et al., Proc. Natl. Acad. Sci. USA 82, 6148-615 [1985]); gene targeting in embryonic stem cells (Thompson et al., Cell 56, 313-321 [1989]); electroporation of embryos (Lo, Mol. Cel. Biol. 3, 1803-1814 [1983]); sperm-mediated gene transfer (Lavitrano et al., Cell 57, 717-73 [1989]). For review, see, for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA 89, 6232-636 (1992).

The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry.

The animals may be further examined for signs of immune disease pathology, for example by histological examination to determine infiltration of immune cells into specific tissues. Blocking experiments can also be performed in which the transgenic animals are treated with the compounds of the invention to determine the extent of the T cell proliferation stimulation or inhibition of the compounds. In these experiments, blocking antibodies which bind to the PRO polypeptide, prepared as described above, are administered to the animal and the effect on immune function is determined.

Alternatively, “knock out” animals can be constructed which have a defective or altered gene encoding a polypeptide identified herein, as a result of homologous recombination between the endogenous gene encoding the polypeptide and altered genomic DNA encoding the same polypeptide introduced into an embryonic cell of the animal. For example, cDNA encoding a particular polypeptide can be used to clone genomic DNA encoding that polypeptide in accordance with established techniques. A portion of the genomic DNA encoding a particular polypeptide can be deleted or replaced with another gene, such as a gene encoding a selectable marker which can be used to monitor integration. Typically, several kilobases of unaltered flanking DNA (both at the 5′ and 3′ ends) are included in the vector [see e.g., Thomas and Capecchi, Cell, 51:503 (1987) for a description of homologous recombination vectors]. The vector is introduced into an embryonic stem cell line (e.g., by electroporation) and cells in which the introduced DNA has homologously recombined with the endogenous DNA are selected [see e.g., Li et al., Cell, 69:915 (1992)]. The selected cells are then injected into a blastocyst of an animal (e.g., a mouse or rat) to form aggregation chimeras [see e.g., Bradley, in Teratocarcinomas and Embryonic Stem Cells: A Practical Approach, E. J. Robertson, ed. (IRL, Oxford, 1987), pp. 113-152]. A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the polypeptide.

I. ImmunoAdjuvant Therapy

In one embodiment, the immunostimulating compounds of the invention can be used in immunoadjuvant therapy for the treatment of tumors (cancer). It is now well established that T cells recognize human tumor specific antigens. One group of tumor antigens, encoded by the MAGE, BAGE and GAGE families of genes, are silent in all adult normal tissues, but are expressed in significant amounts in tumors, such as melanomas, lung tumors, head and neck tumors, and bladder carcinomas. DeSmet, C. et al., (1996) Proc. Natl. Acad. Sci. USA, 93:7149. It has been shown that costimulation of T cells induces tumor regression and an antitumor response both in vitro and in vivo. Melero, I. et al., Nature Medicine (1997) 3:682; Kwon, E. D. et al., Proc. Natl. Acad. Sci. USA (1997) 94: 8099; Lynch, D. H. et al, Nature Medicine (1997) 3:625; Finn, O. J. and Lotze, M. T., J. Immunol. (1998) 21:114. The stimulatory compounds of the invention can be administered as adjuvants, alone or together with a growth regulating agent, cytotoxic agent or chemotherapeutic agent, to stimulate T cell proliferation/activation and an antitumor response to tumor antigens. The growth regulating, cytotoxic, or chemotherapeutic agent may be administered in conventional amounts using known administration regimes. Immunostimulating activity by the compounds of the invention allows reduced amounts of the growth regulating, cytotoxic, or chemotherapeutic agents thereby potentially lowering the toxicity to the patient.

J. Screening Assays for Drug Candidates

Screening assays for drug candidates are designed to identify compounds that bind to or complex with the polypeptides encoded by the genes identified herein or a biologically active fragment thereof, or otherwise interfere with the interaction of the encoded polypeptides with other cellular proteins. Such screening assays will include assays amenable to high-throughput screening of chemical libraries, making them particularly suitable for identifying small molecule drug candidates. Small molecules contemplated include synthetic organic or inorganic compounds, including peptides, preferably soluble peptides, (poly)peptide-immunoglobulin fusions, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. The assays can be performed in a variety of formats, including protein-protein binding assays, biochemical screening assays, immunoassays and cell based assays, which are well characterized in the art. All assays are common in that they call for contacting the drug candidate with a polypeptide encoded by a nucleic acid identified herein under conditions and for a time sufficient to allow these two components to interact.

In binding assays, the interaction is binding and the complex formed can be isolated or detected in the reaction mixture. In a particular embodiment, the polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the polypeptide and drying. Alternatively, an immobilized antibody, e.g., a monoclonal antibody, specific for the polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labelled antibody specifically binding the immobilized complex.

If the candidate compound interacts with but does not bind to a particular protein encoded by a gene identified herein, its interaction with that protein can be assayed by methods well known for detecting protein-protein interactions. Such assays include traditional approaches, such as, cross-linking, co-immunoprecipitation, and co-purification through gradients or chromatographic columns. In addition, protein-protein interactions can be monitored by using a yeast-based genetic system described by Fields and co-workers [Fields and Song, Nature (London) 340, 245-246 (1989); Chien et al., Proc. Natl. Acad. Sci. USA 88, 9578-9582 (1991)] as disclosed by Chevray and Nathans, Proc. Natl. Acad. Sci. USA 89, 5789-5793 (1991). Many transcriptional activators, such as yeast GAL4, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

In order to find compounds that interfere with the interaction of a gene identified herein and other intra- or extracellular components can be tested, a reaction mixture is usually prepared containing the product of the gene and the intra- or extracellular component under conditions and for a time allowing for the interaction and binding of the two products. To test the ability of a test compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described above. The formation of a complex in the control reaction(s) but not in the reaction mixture containing the test compound indicates that the test compound interferes with the interaction of the test compound and its reaction partner.

K. Compositions and Methods for the Treatment of Immune Related Diseases

The compositions useful in the treatment of immune related diseases include, without limitation, proteins, antibodies, small organic molecules, peptides, phosphopeptides, antisense and ribozyme molecules, triple helix molecules, etc. that inhibit or stimulate immune function, for example, T cell proliferation/activation, lymphokine release, or immune cell infiltration.

For example, antisense RNA and RNA molecules act to directly block the translation of mRNA by hybridizing to targeted mRNA and preventing protein translation. When antisense DNA is used, oligodeoxyribonucleotides derived from the translation initiation site, e.g., between about −10 and +10 positions of the target gene nucleotide sequence, are preferred.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology 4, 469-471 (1994), and PCT publication No. WO 97/33551 (published Sep. 18, 1997).

Nucleic acid molecules in triple helix formation used to inhibit transcription should be single-stranded and composed of deoxynucleotides. The base composition of these oligonucleotides is designed such that it promotes triple helix formation via Hoogsteen base pairing rules, which generally require sizeable stretches of purines or pyrimidines on one strand of a duplex. For further details see, e.g., PCT publication No. WO 97/33551, supra.

These molecules can be identified by any or any combination of the screening assays discussed above and/or by any other screening techniques well known for those skilled in the art.

L. Anti-PRO Antibodies

The present invention further provides anti-PRO antibodies. Exemplary antibodies include polyclonal, monoclonal, humanized, bispecific, and heteroconjugate antibodies.

1. Polyclonal Antibodies

The anti-PRO antibodies may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the PRO polypeptide or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-PRO antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the PRO polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies [Kozbor, J. Immunol., 133:3001 (1984); Brodeur et al., Monoclonal Antibody Production Techniques and Applications, Marcel Dekker, Inc., New York, (1987) pp. 51-63].

The culture medium in which the hybridoma cells are cultured can then be assayed for the presence of monoclonal antibodies directed against PRO. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese [symbol] ster ovary (CHO) cells, or myeloma cells that do not otherwise produce immunoglobulin protein, to [symbol] the synthesis of monoclonal antibodies in the recombinant host cells. The DNA also may be modified, for example, by substituting the coding sequence for human heavy and light chain constant [symbol] ains in place of the homologous murine sequences [U.S. Pat. No. 4,816,567; Morrison et al., supra] or [symbol] covalently joining to the immunoglobulin coding sequence all or part of the coding sequence for a non- [symbol] immunoglobulin polypeptide. Such a non-immunoglobulin polypeptide can be substituted for the constant [symbol] ains of an antibody of the invention, or can be substituted for the variable domains of one antigen- [symbol] mbining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Human and Humanized Antibodies

The anti-PRO antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

The antibodies may also be affinity matured using known selection and/or mutagenesis methods as described above. Preferred affinity matured antibodies have an affinity which is five times, more preferably 10 times, even more preferably 20 or 30 times greater than the starting antibody (generally murine, humanized or human) from which the matured antibody is prepared.

4. Bispecific Antibodies

Bispecific antibodies are monoclonal, preferably human or humanized, antibodies that have binding specificities for at least two different antigens. In the present case, one of the binding specificities is for the PRO, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities [Milstein and Cuello, Nature, 305:537-539 (1983)]. Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies may bind to two different epitopes on a given PRO polypeptide herein. Alternatively, an anti-PRO polypeptide arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PRO polypeptide. Bispecific antibodies may also be used to localize cytotoxic agents to cells which express a particular PRO polypeptide. These antibodies possess a PRO-binding arm and an arm which binds a cytotoxic agent or a radionuclide chelator, such as EOTUBE, DPTA, DOTA, or TETA. Another bispecific antibody of interest binds the PRO polypeptide and further binds tissue factor (TF).

5. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

6. Effector Function Engineering

It may be desirable to modify the antibody of the invention with respect to effector function, so as to enhance, e.g., the effectiveness of the antibody in treating cancer. For example, cysteine residue(s) may be introduced into the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity may also be prepared using heterobifunctional cross-linkers as described in Wolff et al. Cancer Research, 53: 2560-2565 (1993). Alternatively, an antibody can be engineered that has dual Fc regions and may thereby have enhanced complement lysis and ADCC capabilities. See Stevenson et al., Anti-Cancer Drug Design, 3: 219-230 (1989).

7. Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol)propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

8. Immunoliposomes

The antibodies disclosed herein may also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19):1484 (1989).

M. Pharmaceutical Compositions

The active PRO molecules of the invention (e.g., PRO polypeptides, anti-PRO antibodies, and/or variants of each) as well as other molecules identified by the screening assays disclosed above, can be administered for the treatment of immune related diseases, in the form of pharmaceutical compositions.

Therapeutic formulations of the active PRO molecule, preferably a polypeptide or antibody of the invention, are prepared for storage by mixing the active molecule having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Compounds identified by the screening assays disclosed herein can be formulated in an analogous manner, using standard techniques well known in the art.

Lipofections or liposomes can also be used to deliver the PRO molecule into cells. Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90, 7889-7893 [1993]).

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active PRO molecules may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations or the PRO molecules may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L- [symbol] mate, non-degradable ethylene-vinyl acetate, degradable lactic acid-glycolic acid copolymers such as [symbol] UPRON DEPOT™ (injectable microspheres composed of lactic acid-glycolic acid copolymer and [symbol] prolide acetate), and poly-D-(−)-3-hydroxybutyric acid. While polymers such as ethylene-vinyl acetate [symbol] lactic acid-glycolic acid enable release of molecules for over 100 days, certain hydrogels release proteins [symbol] shorter time periods. When encapsulated antibodies remain in the body for a long time, they may [symbol] or aggregate as a result of exposure to moisture at 37° C., resulting in a loss of biological activity [symbol] possible changes in immunogenicity. Rational strategies can be devised for stabilization depending on [symbol] mechanism involved. For example, if the aggregation mechanism is discovered to be intermolecular S—S [symbol] formation through thio-disulfide interchange, stabilization may be achieved by modifying sulfhydryl residues, lyophilizing from acidic solutions, controlling moisture content, using appropriate additives, and developing specific polymer matrix compositions.

N. Methods of Treatment

It is contemplated that the polypeptides, antibodies and other active compounds of the present invention may be used to treat various immune related diseases and conditions, such as T cell mediated diseases, including those characterized by infiltration of inflammatory cells into a tissue, stimulation of T-cell proliferation, inhibition of T-cell proliferation, increased or decreased vascular permeability or the inhibition thereof.

Exemplary conditions or disorders to be treated with the polypeptides, antibodies and other compounds of the invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In systemic lupus erythematosus, the central mediator of disease is the production of auto-reactive antibodies to self proteins/tissues and the subsequent generation of immune-mediated inflammation. Antibodies either directly or indirectly mediate tissue injury. Though T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that mainly involves the synovial membrane of multiple joints with resultant injury to the articular cartilage. The pathogenesis is T lymphocyte dependent and is associated with the production of rheumatoid factors, auto-antibodies directed against self IgG, with the resultant formation of immune complexes that attain high levels in joint fluid and blood. These complexes in the joint may induce the marked infiltrate of lymphocytes and monocytes into the synovium and subsequent marked synovial changes; the joint space/fluid if infiltrated by similar cells with the addition of numerous neutrophils. Tissues affected are primarily the joints, often in symmetrical pattern. However, extra-articular disease also occurs in two major forms. One form is the development of extra-articular lesions with ongoing progressive joint disease and typical lesions of pulmonary fibrosis, vasculitis, and cutaneous ulcers. The second form of extra-articular disease is the so called Felty's syndrome which occurs late in the RA disease course, sometimes after joint disease has become quiescent, and involves the presence of neutropenia, thrombocytopenia and splenomegaly. This can be accompanied by vasculitis in multiple organs with formations of infarcts, skin ulcers and gangrene. Patients often also develop rheumatoid nodules in the subcutis tissue overlying affected joints; the nodules late stage have necrotic centers surrounded by a mixed inflammatory cell infiltrate. Other manifestations which can occur in RA include: pericarditis, pleuritis, coronary arteritis, intestitial pneumonitis with pulmonary fibrosis, keratoconjunctivitis sicca, and rhematoid nodules.

Juvenile chronic arthritis is a chronic idiopathic inflammatory disease which begins often at less than 16 years of age. Its phenotype has some similarities to RA; some patients which are rhematoid factor positive are classified as juvenile rheumatoid arthritis. The disease is sub-classified into three major categories: pauciarticular, polyarticular, and systemic. The arthritis can be severe and is typically destructive and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin; likely this is induced by an active inflammatory process. Scleroderma can be localized or systemic; vascular lesions are common and endothelial cell injury in the microvasculature is an early and important event in the development of systemic sclerosis; the vascular injury may be immune mediated. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs involved include: the gastrointestinal tract: smooth muscle atrophy and fibrosis resulting in abnormal peristalsis/motility; kidney: concentric subendothelial intimal proliferation affecting small arcuate and interlobular arteries with resultant reduced renal cortical blood flow, results in proteinuria, azotemia and hypertension; skeletal muscle: atrophy, interstitial fibrosis; inflammation; lung: interstitial pneumonitis and interstitial fibrosis; and heart: contraction band necrosis, scarring/fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components, proteins and RNA's, involved in protein synthesis.

Sjögren's syndrome is due to immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc., particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epitheloid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Type I diabetes mellitus or insulin-dependent diabetes is the autoimmune destruction of pancreatic islet β cells; this destruction is mediated by auto-antibodies and auto-reactive T cells. Antibodies to insulin or the insulin receptor can also produce the phenotype of insulin-non-responsiveness.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions.

Demyelinating diseases of the central and peripheral nervous systems, including Multiple Sclerosis; idiopathic demyelinating polyneuropathy or Guillain-Barré syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple Sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+ T lymphocytes are the predominant cell type at lesions. The mechanism of oligodendrocyte cell death and subsequent demyelination is not known but is likely T lymphocyte driven.

Inflammatory and Fibrotic Lung Disease, including Eosinophilic Pneumonias; Idiopathic Pulmonary Fibrosis, and Hypersensitivity Pneumonitis may involve a disregulated immune-inflammatory response. Inhibition of that response would be of therapeutic benefit.

Autoimmune or Immune-mediated Skin Disease including Bullous Skin Diseases, Erythema Multiforme, and Contact Dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Allergic diseases, including asthma; allergic rhinitis; atopic dermatitis; food hypersensitivity; and urticaria are T lymphocyte dependent. These diseases are predominantly mediated by T lymphocyte induced inflammation, IgE mediated-inflammation or a combination of both.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e., as from chemotherapy) immunodeficiency, and neoplasia.

It has been demonstrated that some human cancer patients develop an antibody and/or T lymphocyte response to antigens on neoplastic cells. It has also been shown in animal models of neoplasia that enhancement of the immune response can result in rejection or regression of that particular neoplasm. Molecules that enhance the T lymphocyte response in the MLR have utility in vivo in enhancing the immune response against neoplasia. Molecules which enhance the T lymphocyte proliferative response in the MLR (or small molecule agonists or antibodies that affected the same receptor in an agonistic fashion) can be used therapeutically to treat cancer. Molecules that inhibit the lymphocyte response in the MLR also function in vivo during neoplasia to suppress the immune response to a neoplasm; such molecules can either be expressed by the neoplastic cells themselves or their expression can be induced by the neoplasm in other cells. Antagonism of such inhibitory molecules (either with antibody, small molecule antagonists or other means) enhances immune-mediated tumor rejection.

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

The compounds of the present invention, e.g., polypeptides or antibodies, are administered to a mammal, preferably a human, in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation (intranasal, intrapulmonary) routes. Intravenous or inhaled administration of polypeptides and antibodies is preferred.

In immunoadjuvant therapy, other therapeutic regimens, such administration of an anticancer agent, may be combined with the administration of the proteins, antibodies or compounds of the instant invention. For example, the patient to be treated with a the immunoadjuvant of the invention may also receive an anti-cancer agent (chemotherapeutic agent) or radiation therapy. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992). The chemotherapeutic agent may precede, or follow administration of the immunoadjuvant or may be given simultaneously therewith. Additionally, an anti-estrogen compound such as tamoxifen or an anti-progesterone such as onapristone (see, EP 616812) may be given in dosages known for such molecules.

It may be desirable to also administer antibodies against other immune disease associated or tumor associated antigens, such as antibodies which bind to CD20, CD11a, CD18, ErbB2, EGFR, ErbB3, ErbB4, or vascular endothelial factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the PRO polypeptides are coadministered with a growth inhibitory agent. For example, the growth inhibitory agent may be administered first, followed by a PRO polypeptide. However, simultaneous administration or administration first is also contemplated. Suitable dosages for the growth inhibitory agent are those presently used and may be lowered due to the combined action (synergy) of the growth inhibitory agent and the PRO polypeptide.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the attending physician. The compound is suitably administered to the patient at one time or over a series of treatments.

For example, depending on the type and severity of the disease, about 1 μg/kg to 15 mg/kg (e.g., 0.1-20 mg/kg) of polypeptide or antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

O. Articles of Manufacture

In another embodiment of the invention, an article of manufacture containing materials (e.g., comprising a PRO molecule) useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is usually a polypeptide or an antibody of the invention. An instruction or label on, or associated with, the container indicates that the composition is used for diagnosing or treating the condition [symbol] choice. The article of manufacture may further comprise a second container comprising a [symbol] pharmaceutically-acceptable buffer, such as phosphate-buffered saline, Ringer's solution and dextrose [symbol] on. It may further include other materials desirable from a commercial and user standpoint, including [symbol] buffers, diluents, filters, needles, syringes, and package inserts with instructions for use.

P. Diagnosis and Prognosis of Immune Related Disease

Cell surface proteins, such as proteins which are overexpressed in certain immune related diseases, [symbol] excellent targets for drug candidates or disease treatment. The same proteins along with secreted [symbol] ins encoded by the genes amplified in immune related disease states find additional use in the diagnosis and prognosis of these diseases. For example, antibodies directed against the protein products of genes amplified in multiple sclerosis, rheumatoid arthritis, or another immune related disease, can be used as diagnostics or prognostics.

For example, antibodies, including antibody fragments, can be used to qualitatively or quantitatively detect the expression of proteins encoded by amplified or overexpressed genes (“marker gene products”). The antibody preferably is equipped with a detectable, e.g., fluorescent label, and binding can be monitored by light microscopy, flow cytometry, fluorimetry, or other techniques known in the art. These techniques are particularly suitable, if the overexpressed gene encodes a cell surface protein. Such binding assays are performed essentially as described above.

In situ detection of antibody binding to the marker gene products can be performed, for example, by immunofluorescence or immunoelectron microscopy. For this purpose, a histological specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Example 1

### Microarray Analysis of Stimulated T-Cells

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (in this instance, activated CD4+ T cells) sample is greater than hybridization signal of a probe from a control (in this instance, non-stimulated CD4+ T cells) sample, the gene or genes overexpressed in the test tissue are identified. The implication of this result is that an overexpressed protein in a test tissue is useful not only as a diagnostic marker for the presence of the disease condition, but also as a therapeutic target for treatment of the disease condition.

The methodology of hybridization of nucleic acids and microarray technology is well known in the art. In one example, the specific preparation of nucleic acids for hybridization and probes, slides, and hybridization conditions are all detailed in PCT Patent Application Serial No. PCT/US01/10482, filed on Mar. 30, 2001 and which is herein incorporated by reference.

The specific conditions for this set of experiments began by collecting 100 ml of fresh blood from donors. Peripheral blood mononuclear cells (PBMC) were isolated with LSM (ficol) (ICN Biomedicals) by step gradient separation. Monocytes were depleted by adherence to culture flask. CD45 RA and CD45 RO high cells were sorted by FACS with additional gating on lymphocytes by forward and side scatter. Cells of intermediate expression of either CD45RA or CD45 RO were not collected. Sorting was verified by re-FACS of samples of the sorted population and found to be approximately 99% correctly sorted. Cells were cultured for 16 hours in RPMI 1640, 10% heat inactivated FBS, 100 units/mL of Penicillin, 100 mg/mL of streptomycin, 2 mM L-glutamine and IL-2 (100 U/ml) and in the presence or absence of plate bound anti-CD3 (10 ug/ml) and soluble anti-CD28 (10 ug/ml). The activation status of the cells was monitored by FACS for cell surface expression of CD69 and CD25. Cells were then pelleted and RNA isolated by Qiagen miniprep and analysis run on Affimax™ (Affymetrix Inc. Santa Clara, Calif.) microarray chips. Non-stimulated (resting) cells were harvested immediately after purification, and subjected to the same analysis. Genes were compared whose expression was upregulated at either of the two timepoints in activated vs. resting cells

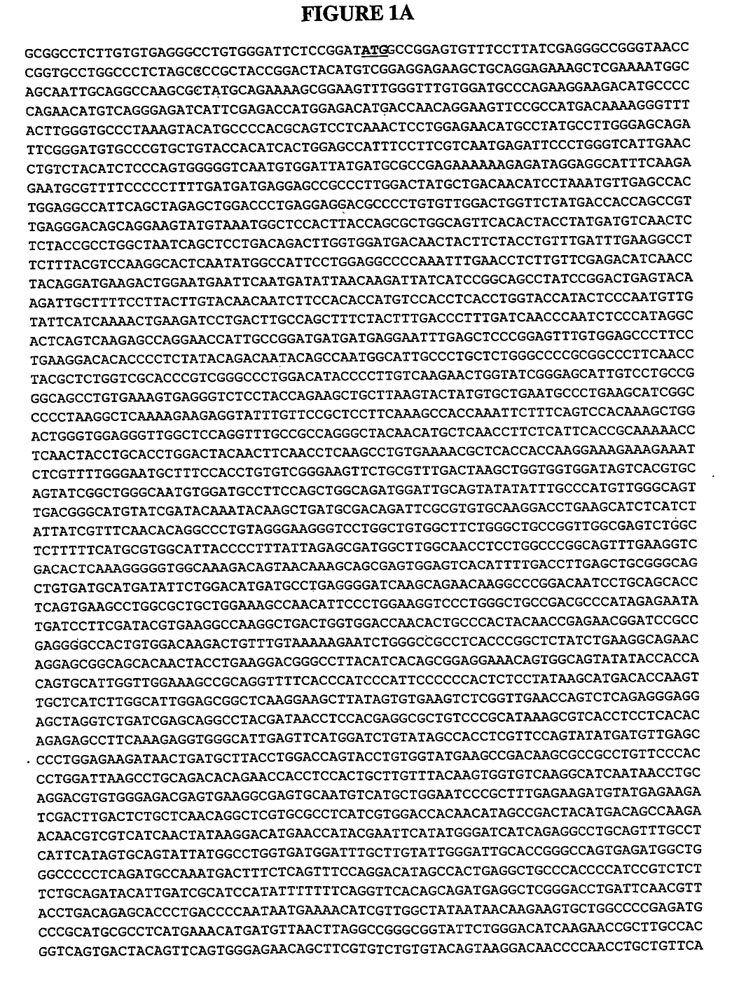

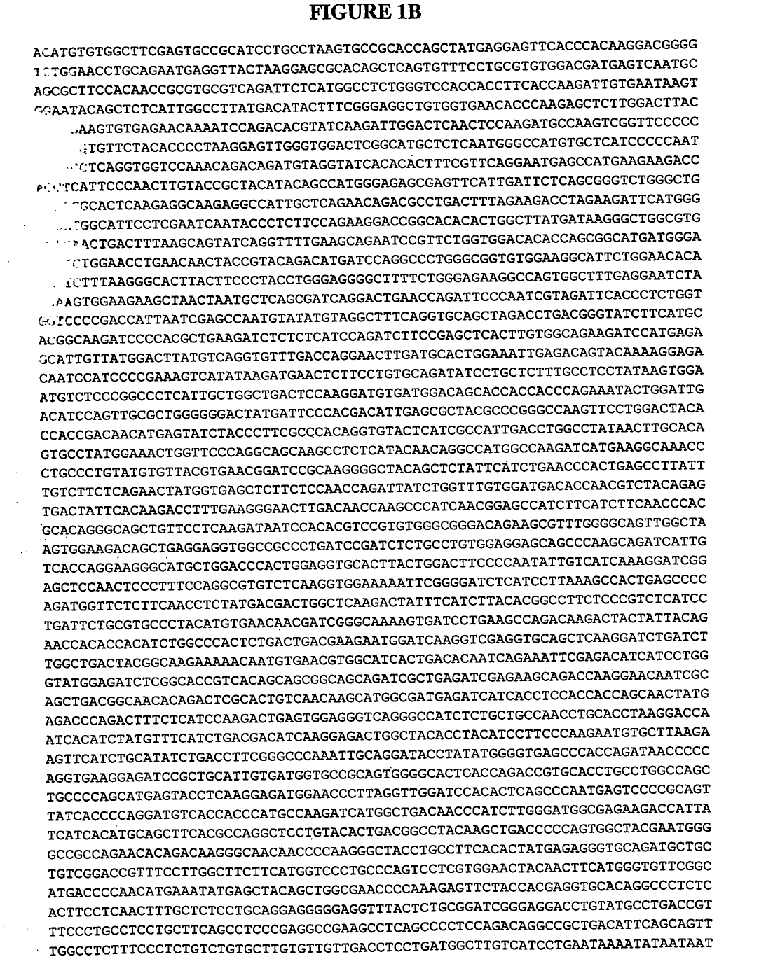

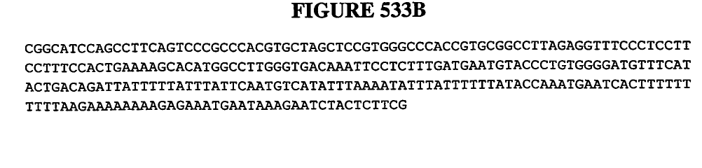

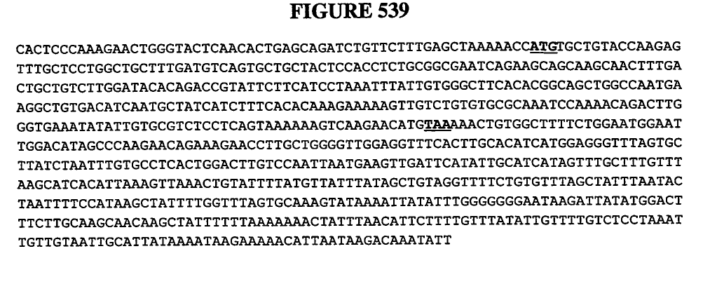

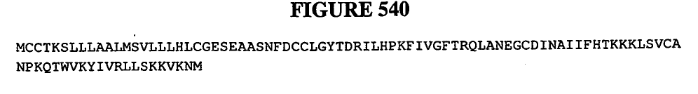

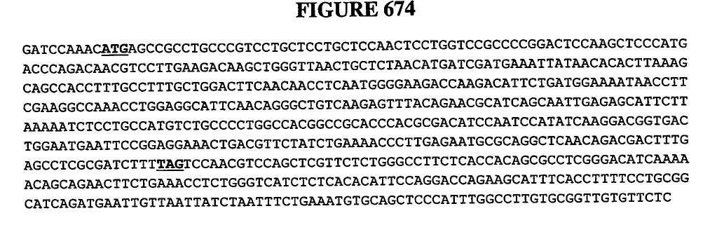

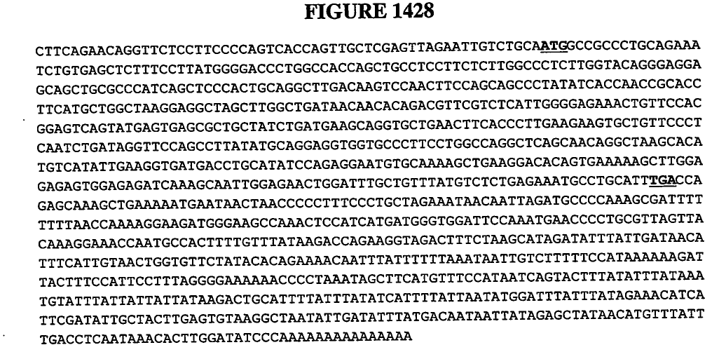

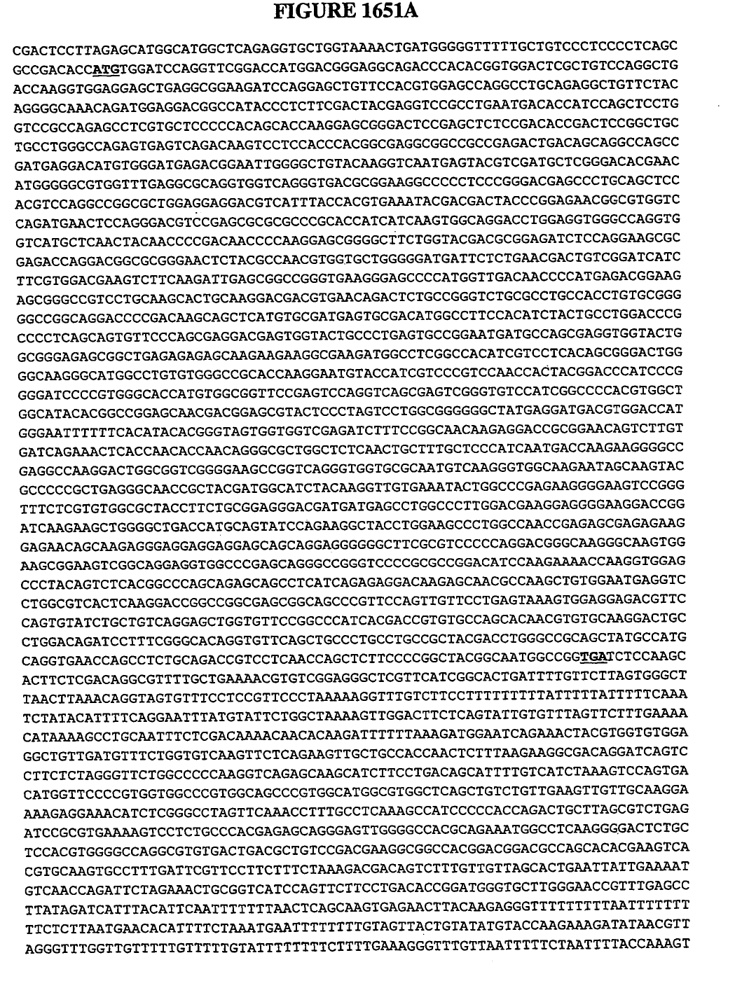

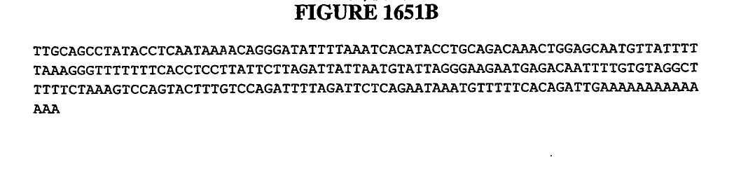

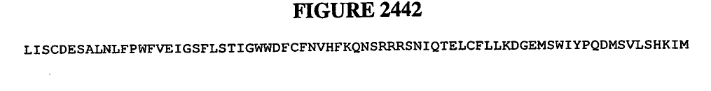

Below are the results of these experiments, demonstrating that various PRO polypeptides of the present invention are significantly differentially expressed in isolated CD45RO activated by anti-CD3/anti-CD28 as compared to: isolated resting CD45RO, isolated resting CD45RA and isolated CD45RA activated by anti-CD3/anti-CD28 cells. As described above, these data demonstrate that the PRO polypeptides of the present invention are useful not only as diagnostic markers for the presence of one or more immune disorders, but also serve as therapeutic targets for the treatment of those immune disorders. The FIGS. 1-2442 show the nucleic acids of the invention and their encoded PRO polypeptides. The nucleic acids and encoded proteins of FIG. 533, FIG. 539, FIG. 674, FIG. 877, FIG. 885, FIG. 1135, FIG. 1428, FIG. 1651 and FIG. 1859 are significantly overexpressed in activated CD45RO compared to matched isolated resting CD45RO, isolated resting CD45RA cells and activated CD45RA cells.

### Example 2

### Use of PRO as a Hybridization Probe

The following method describes use of a nucleotide sequence encoding PRO as a hybridization probe.

DNA comprising the coding sequence of full-length or mature PRO as disclosed herein is employed as a probe to screen for homologous DNAs (such as those encoding naturally-occurring variants of PRO) in human tissue cDNA libraries or human tissue genomic libraries.

Hybridization and washing of filters containing either library DNAs is performed under the following high stringency conditions. Hybridization of radiolabeled PRO-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PRO can then be identified using standard techniques known in the art.

### Example 3

### Expression of PRO in E. coli

This example illustrates preparation of an unglycosylated form of PRO by recombinant expression in E. coli.

The DNA sequence encoding PRO is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PRO coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PRO protein can then be purified using a metal chelating column under conditions that allow tight binding of the protein.

PRO may be expressed in E. coli in a poly-His tagged form, using the following procedure. The DNA encoding PRO is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) lon galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PRO polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

Many of the PRO polypeptides disclosed herein were successfully expressed as described above.

### Example 4

### Expression of PRO in Mammalian Cells

This example illustrates preparation of a potentially glycosylated form of PRO by recombinant expression in mammalian cells.

The vector, pRK5 (see EP 307,247, published Mar. 15, 1989), is employed as the expression vector. Optionally, the PRO DNA is ligated into pRK5 with selected restriction enzymes to allow insertion of the PRO DNA using ligation methods such as described in Sambrook et al., supra. The resulting vector is called pRK5-PRO.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PRO DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of PRO polypeptide. The cultures containing transfected cells may undergo further incubation (in serum free medium) and the medium is tested in selected bioassays.

In an alternative technique, PRO may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PRO DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PRO can then be concentrated and purified by any selected method, such as dialysis and/or column chromatography.

In another embodiment, PRO can be expressed in CHO cells. The pRK5-PRO can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PRO polypeptide, the culture medium may be replaced with serum free medium. Preferably, the cultures are incubated for about 6 days, and then the conditioned medium is harvested. The medium containing the expressed PRO can then be concentrated and purified by any selected method.

Epitope-tagged PRO may also be expressed in host CHO cells. The PRO may be subcloned out of the pRK5 vector. The subclone insert can undergo PCR to fuse in frame with a selected epitope tag such as a poly-his tag into a Baculovirus expression vector. The poly-his tagged PRO insert can then be subcloned into a SV40 promoter/enhancer containing vector containing a selection marker such as DHFR for selection of stable clones. Finally, the CHO cells can be transfected (as described above) with the SV40 promoter/enhancer containing vector. Labeling may be performed, as described above, to verify expression. The culture medium containing the expressed poly-His tagged PRO can then be concentrated and purified by any selected method, such as by Ni²⁺-chelate affinity chromatography.

PRO may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mL of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3 L production spinner is seeded at 1.2×10⁶ cells/mL. On day 0, pH is determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The [symbol] tioned media is pumped onto a 6 ml Ni-NTA column equilibrated in 20 mM Hepes, pH 7.4, buffer [symbol] ning 0.3 M NaCl and 5 mM imidazole at a flow rate of 4-5 ml/min. at 4° C. After loading, the column [symbol] washed with additional equilibration buffer and the protein eluted with equilibration buffer containing [symbol] M imidazole. The highly purified protein is subsequently desalted into a storage buffer containing 10 [symbol] Hepes, 0.14 M NaCl and 4% mannitol, pH 6.8, with a 25 ml G25 Superfine (Pharmacia) column and [symbol] at −80° C.

Immunoadhesin (Fc-containing) constructs are purified from the conditioned media as follows. The [symbol] conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μl of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

Many of the PRO polypeptides disclosed herein were successfully expressed as described above.

### Example 5

### Expression of PRO in Yeast

The following method describes recombinant expression of PRO in yeast.

First, yeast expression vectors are constructed for intracellular production or secretion of PRO from the ADH2/GAPDH promoter. DNA encoding PRO and the promoter is inserted into suitable restriction enzyme sites in the selected plasmid to direct intracellular expression of PRO. For secretion, DNA encoding PRO can be cloned into the selected plasmid, together with DNA encoding the ADH2/GAPDH promoter, a native PRO signal peptide or other mammalian signal peptide, or, for example, a yeast alpha-factor or invertase secretory signal/leader sequence, and linker sequences (if needed) for expression of PRO.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PRO can subsequently be isolated and purified by removing the yeast cells from the fermentation medium by centrifugation and then concentrating the medium using selected cartridge filters. The concentrate containing PRO may further be purified using selected column chromatography resins.

Many of the PRO polypeptides disclosed herein were successfully expressed as described above.

### Example 6

### Expression of PRO in Baculovirus-Infected Insect Cells

The following method describes recombinant expression of PRO in Baculovirus-infected insect cells.

The sequence coding for PRO is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding PRO or the desired portion of the coding sequence of PRO such as the sequence encoding the extracellular domain of a transmembrane protein or the sequence encoding the mature protein if the protein is extracellular is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PRO can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PRO are pooled and dialyzed against loading buffer.

Alternatively, purification of the IgG tagged (or Fc tagged) PRO can be performed using known chromatography techniques, including for instance, Protein A or protein G column chromatography.

Many of the PRO polypeptides disclosed herein were successfully expressed as described above.

### Example 7

### Preparation of Antibodies that Bind PRO

This example illustrates preparation of monoclonal antibodies which can specifically bind PRO.

Techniques for producing the monoclonal antibodies are known in the art and are described, for instance, in Goding, supra. Immunogens that may be employed include purified PRO, fusion proteins containing PRO, and cells expressing recombinant PRO on the cell surface. Selection of the immunogen can be made by the skilled artisan without undue experimentation.

Mice, such as Balb/c, are immunized with the PRO immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-PRO antibodies.

After a suitable antibody titer has been detected, the animals “positive” for antibodies can be injected with a final intravenous injection of PRO. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PRO. Determination of “positive” hybridoma cells secreting the desired monoclonal antibodies against PRO is within the skill in the art.

The positive hybridoma cells can be injected intraperitoneally into syngeneic Balb/c mice to produce ascites containing the anti-PRO monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 8

### Purification of PRO Polypeptides Using Specific Antibodies

Native or recombinant PRO polypeptides may be purified by a variety of standard techniques in the art of protein purification. For example, pro-PRO polypeptide, mature PRO polypeptide, or pre-PRO polypeptide is purified by immunoaffinity chromatography using antibodies specific for the PRO polypeptide of interest. In general, an immunoaffinity column is constructed by covalently coupling the anti-PRO polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PRO polypeptide by preparing a fraction from cells containing PRO polypeptide in a soluble form. This preparation is derived by solubilization of the whole cell or of a subcellular fraction obtained via differential centrifugation by the addition of detergent or by other methods well known in the art. Alternatively, soluble PRO polypeptide containing a signal sequence may be secreted in useful quantity into the medium in which the cells are grown.

A soluble PRO polypeptide-containing preparation is passed over the immunoaffinity column, and the column is washed under conditions that allow the preferential absorbance of PRO polypeptide (e.g., high ionic strength buffers in the presence of detergent). Then, the column is eluted under conditions that disrupt antibody/PRO polypeptide binding (e.g., a low pH buffer such as approximately pH 2-3, or a high concentration of a chaotrope such as urea or thiocyanate ion), and PRO polypeptide is collected.

### Example 9

### Drug Screening

This invention is particularly useful for screening compounds by using PRO polypeptides or binding fragment thereof in any of a variety of drug screening techniques. The PRO polypeptide or fragment employed in such a test may either be free in solution, affixed to a solid support, borne on a cell surface, or located intracellularly. One method of drug screening utilizes eukaryotic or prokaryotic host cells which are stably transformed with recombinant nucleic acids expressing the PRO polypeptide or fragment. Drugs are screened against such transformed cells in competitive binding assays. Such cells, either in viable or fixed form, can be used for standard binding assays. One may measure, for example, the formation of complexes between PRO polypeptide or a fragment and the agent being tested. Alternatively, one can examine the diminution in complex formation between the PRO polypeptide and its target cell or target receptors caused by the agent being tested.

Thus, the present invention provides methods of screening for drugs or any other agents which can affect a PRO polypeptide-associated disease or disorder. These methods comprise contacting such an agent with an PRO polypeptide or fragment thereof and assaying (I) for the presence of a complex between the agent and the PRO polypeptide or fragment, or (ii) for the presence of a complex between the PRO polypeptide or fragment and the cell, by methods well known in the art. In such competitive binding assays, the PRO polypeptide or fragment is typically labeled. After suitable incubation, free PRO polypeptide or fragment is separated from that present in bound form, and the amount of free or uncomplexed label is a measure of the ability of the particular agent to bind to PRO polypeptide or to interfere with the PRO polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PRO polypeptide, the peptide test compounds are reacted with PRO polypeptide and washed. Bound PRO polypeptide is detected by methods well known in the art. Purified PRO polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PRO polypeptide specifically compete with a test compound for binding to PRO polypeptide or fragments thereof. In this manner, the antibodies can be used to detect the presence of any peptide which shares one or more antigenic determinants with PRO polypeptide.

### Example 10

### Rational Drug Design

The goal of rational drug design is to produce structural analogs of biologically active polypeptide of interest (i.e., a PRO polypeptide) or of small molecules with which they interact, e.g., agonists, antagonists, or inhibitors. Any of these examples can be used to fashion drugs which are more active or stable forms of the PRO polypeptide or which enhance or interfere with the function of the PRO polypeptide in vivo (c.f., Hodgson, Bio/Technology, 2: 19-21 (1991)).

In one approach, the three-dimensional structure of the PRO polypeptide, or of a PRO polypeptide-inhibitor complex, is determined by x-ray crystallography, by computer modeling or, most typically, by a combination of the two approaches. Both the shape and charges of the PRO polypeptide must be ascertained to elucidate the structure and to determine active site(s) of the molecule. Less often, useful information regarding the structure of the PRO polypeptide may be gained by modeling based on the structure of homologous proteins. In both cases, relevant structural information is used to design analogous PRO polypeptide-like molecules or to identify efficient inhibitors. Useful examples of rational drug design may include molecules which have improved activity or stability as shown by Braxton and Wells, Biochemistry, 31:7796-7801 (1992) or which act as inhibitors, agonists, or antagonists of native peptides as shown by Athauda et al., J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PRO polypeptide may be made available to perform such analytical studies as X-ray crystallography. In addition, knowledge of the PRO polypeptide amino acid sequence provided herein will provide guidance to those employing computer modeling techniques in place of or in addition to x-ray crystallography.

The foregoing written specification is considered to be sufficient to enable one skilled in the art to practice the invention. The present invention is not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents. Indeed, various modifications of the invention in addition to those shown and described herein will become apparent to those skilled in the art from the foregoing description and fall within the scope of the appended claims.

What is claimed:

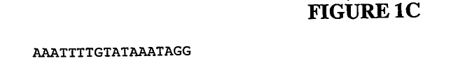

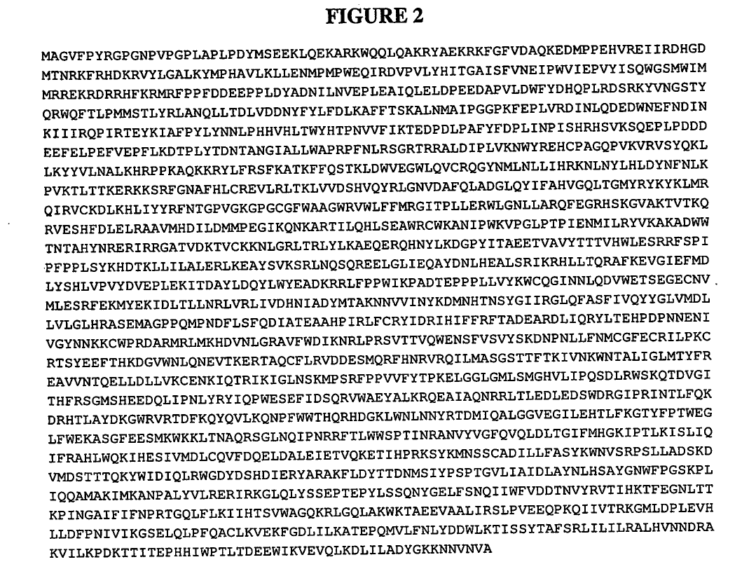

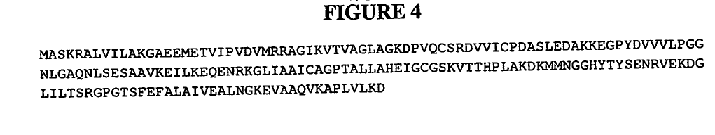

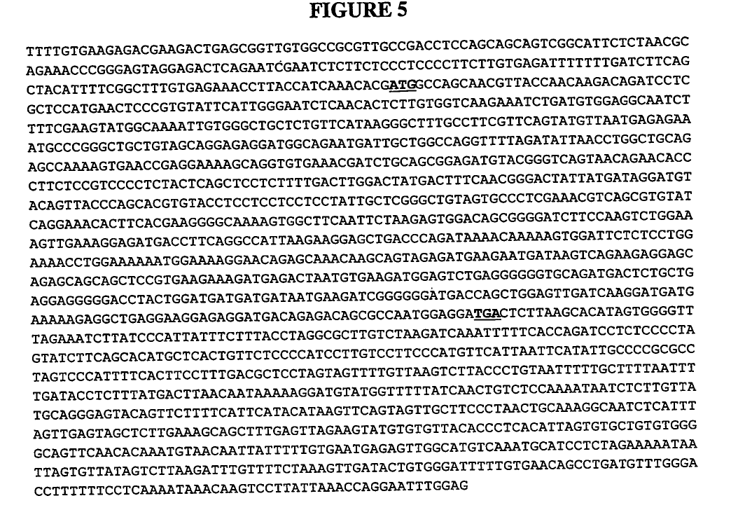

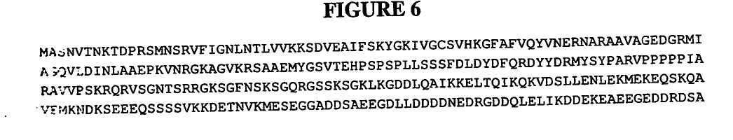

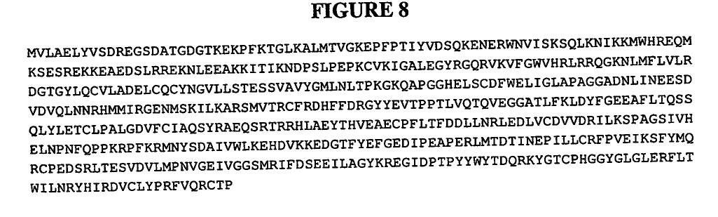

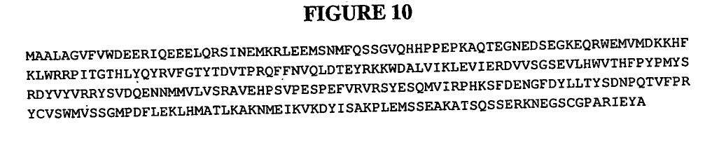

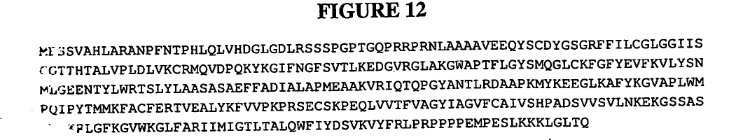

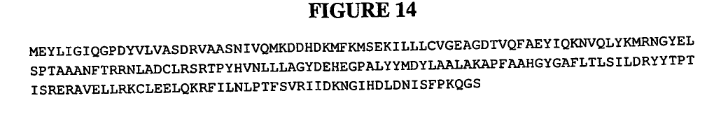

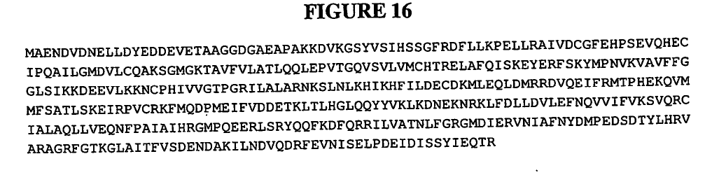

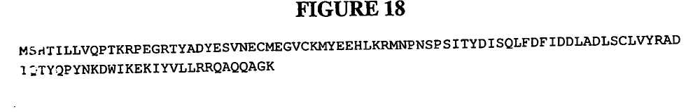

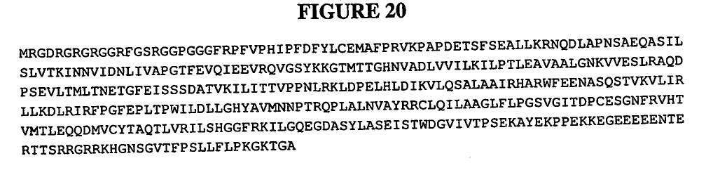

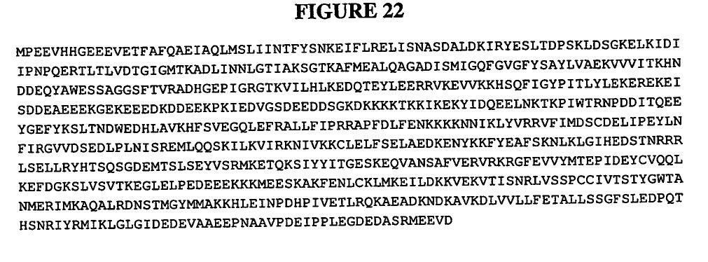

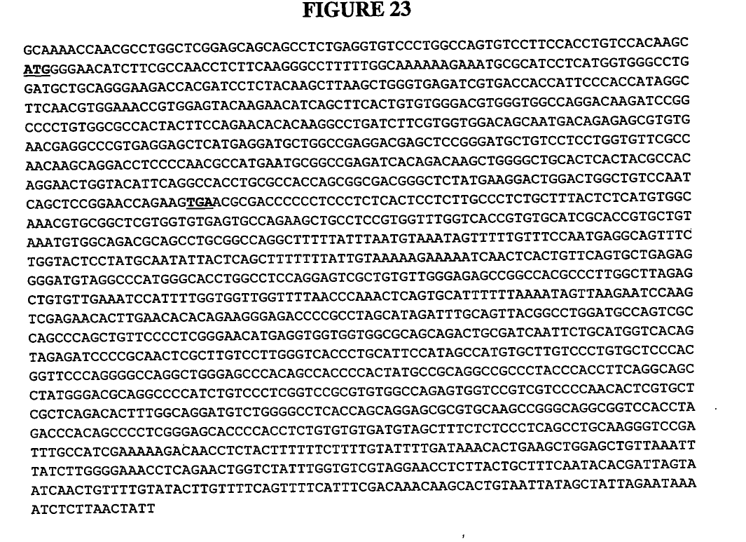

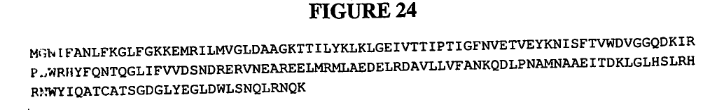

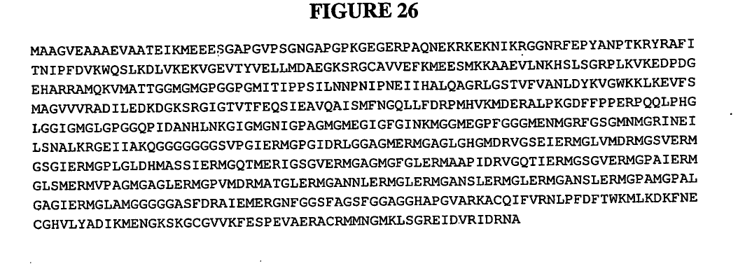

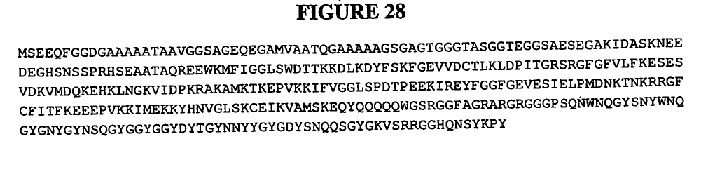

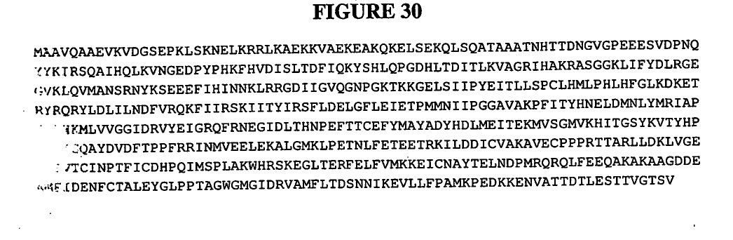

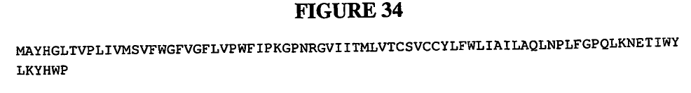

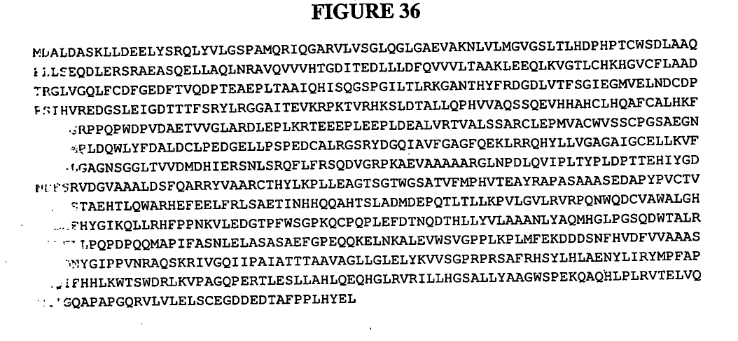

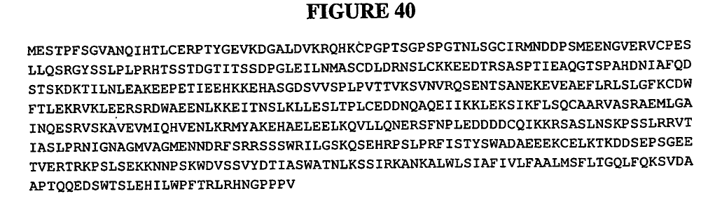

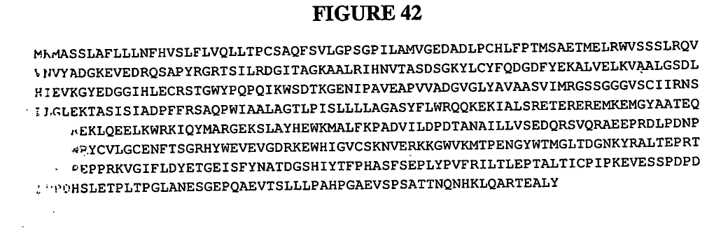

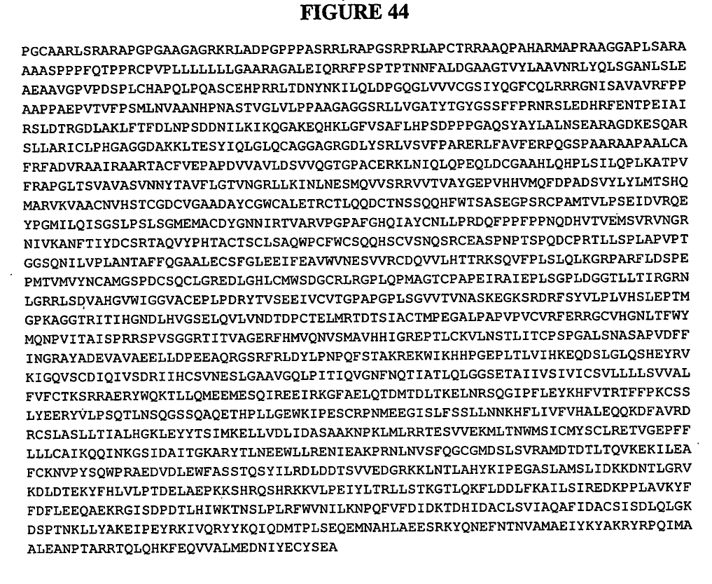

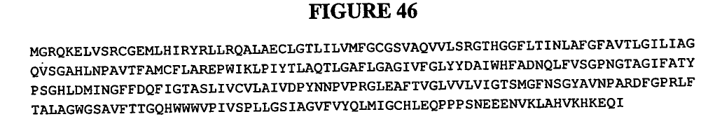

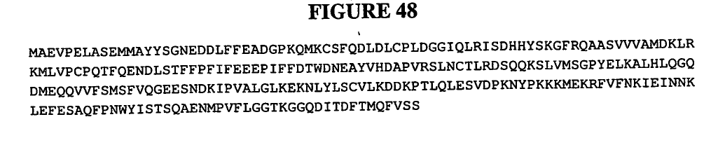

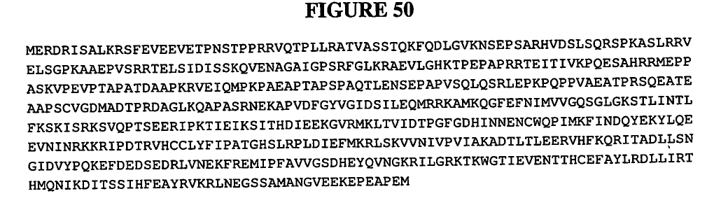

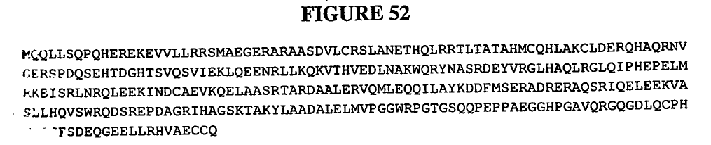

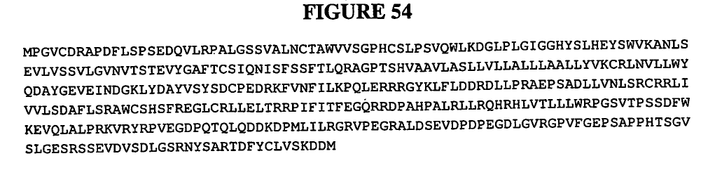

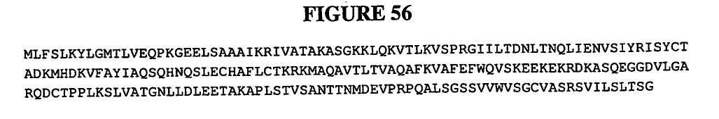

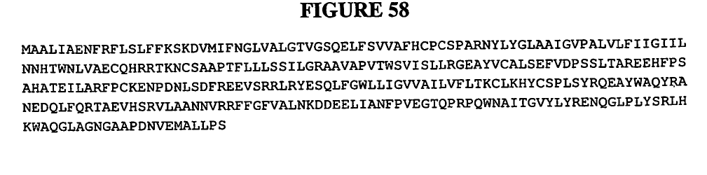

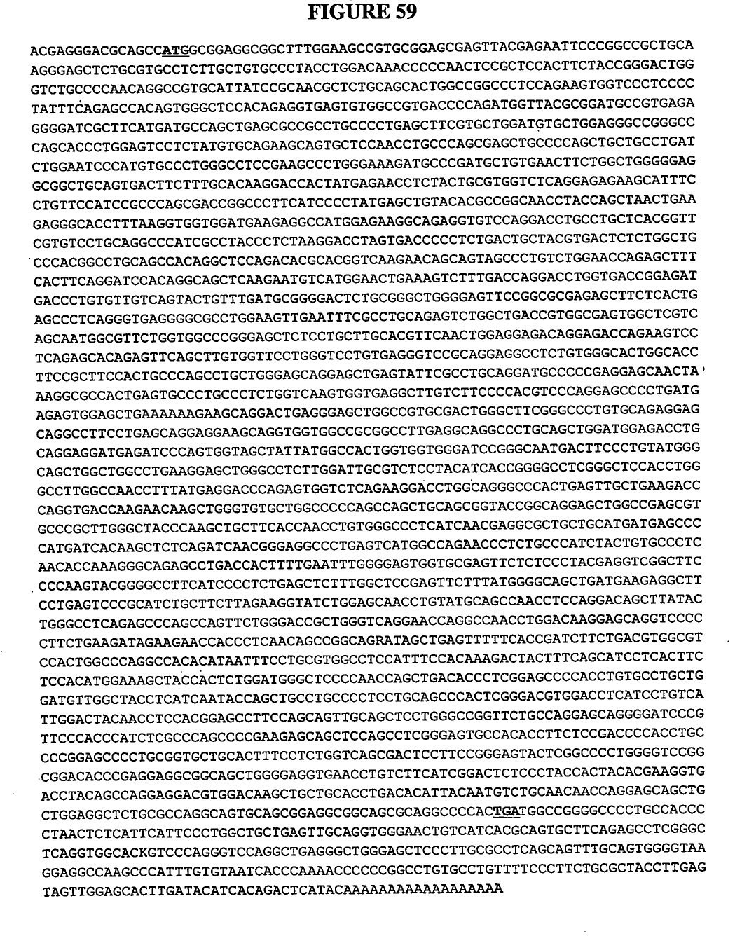

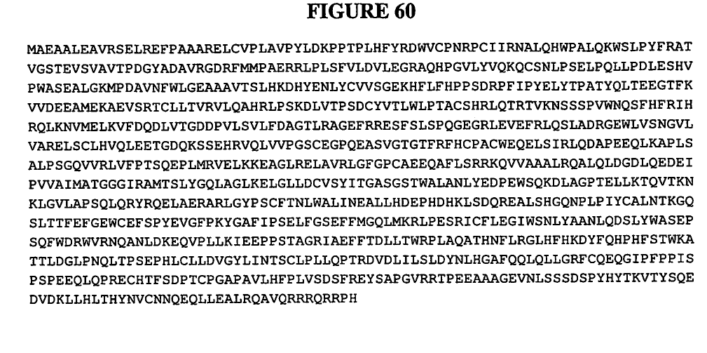

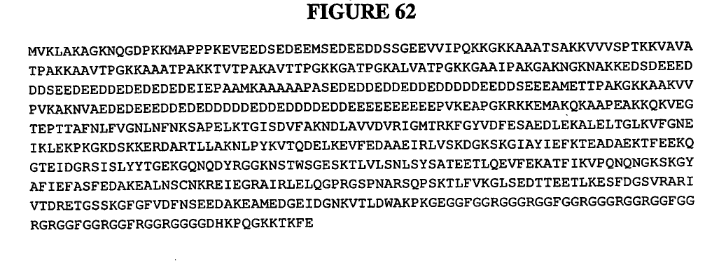

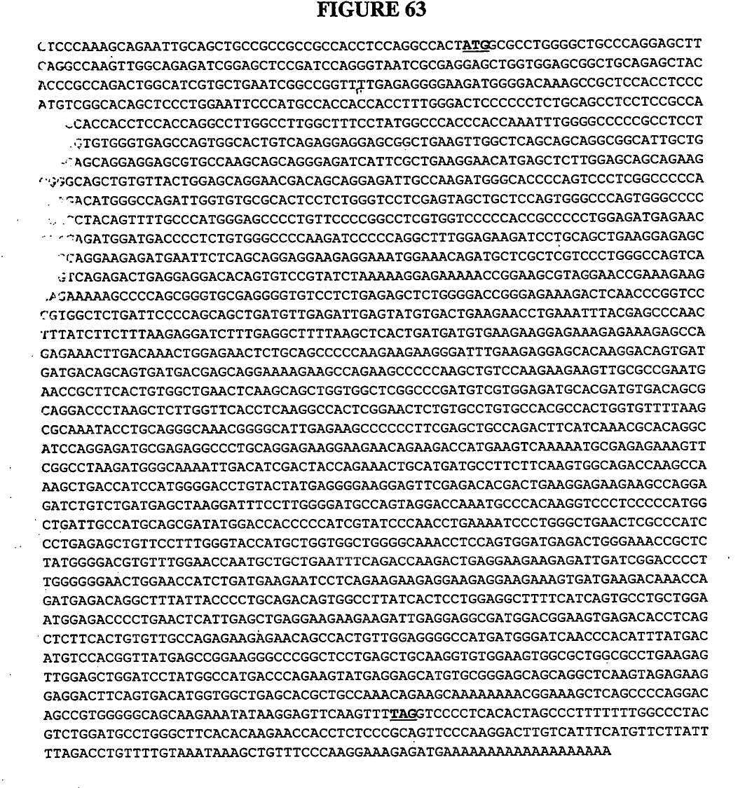

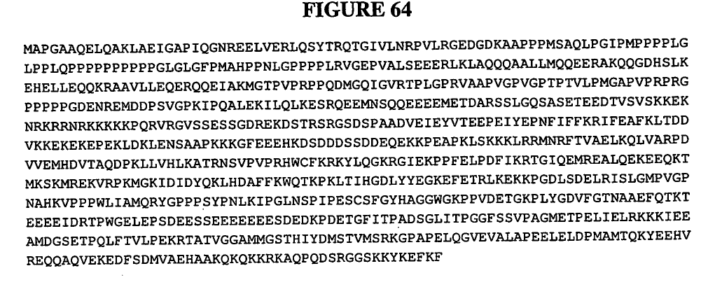

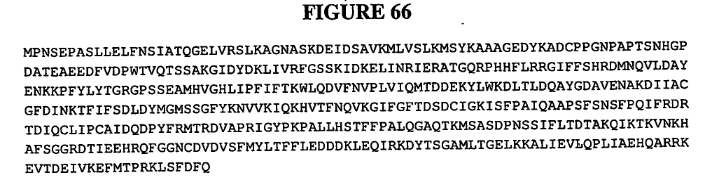

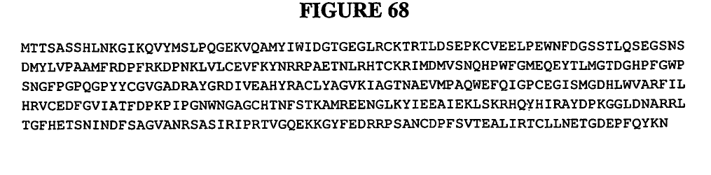

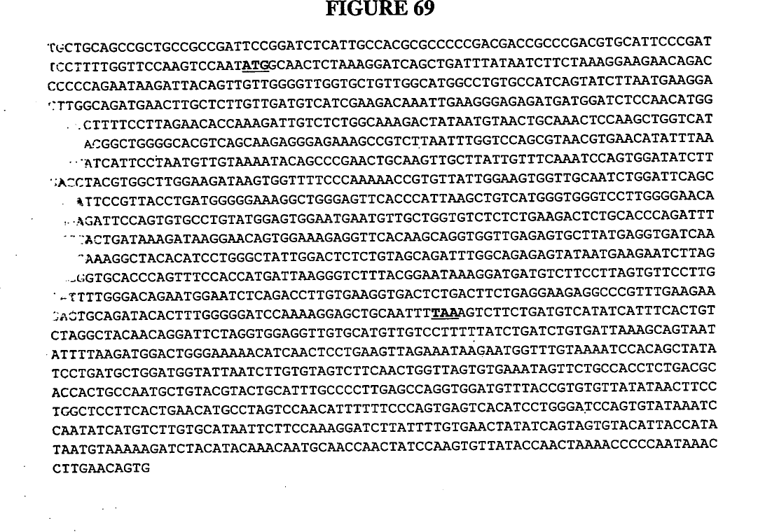

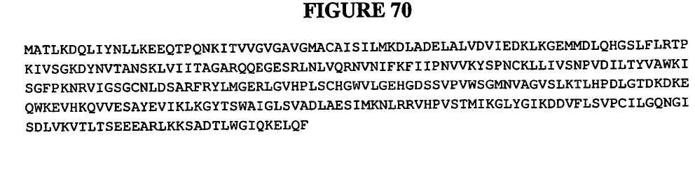

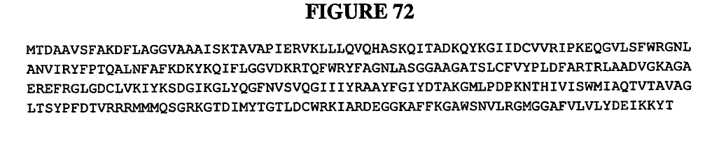

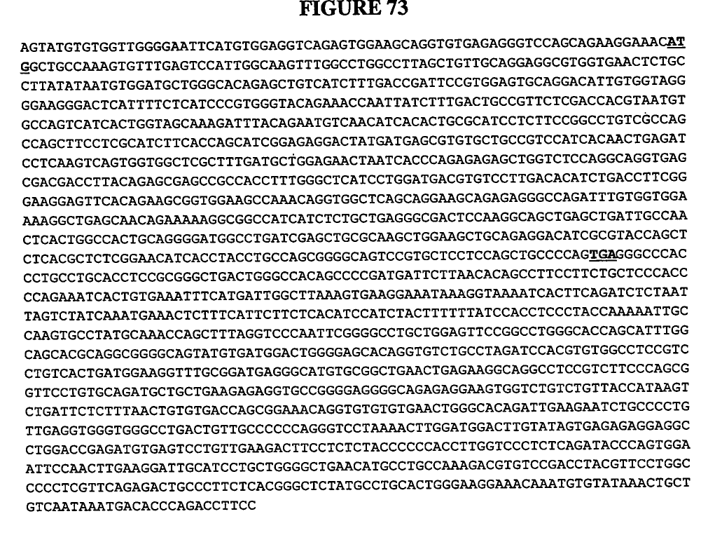

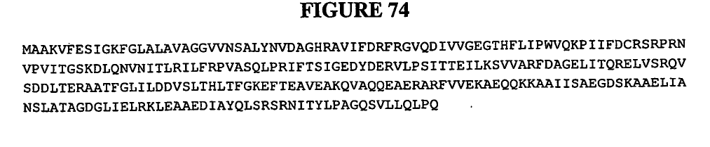

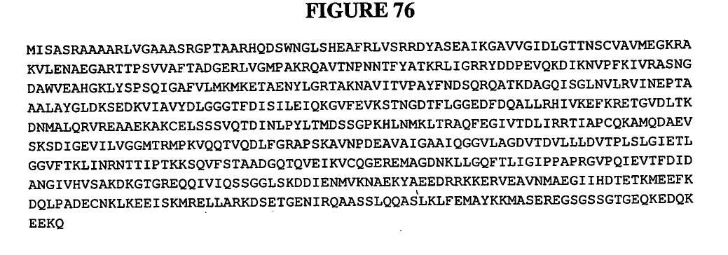

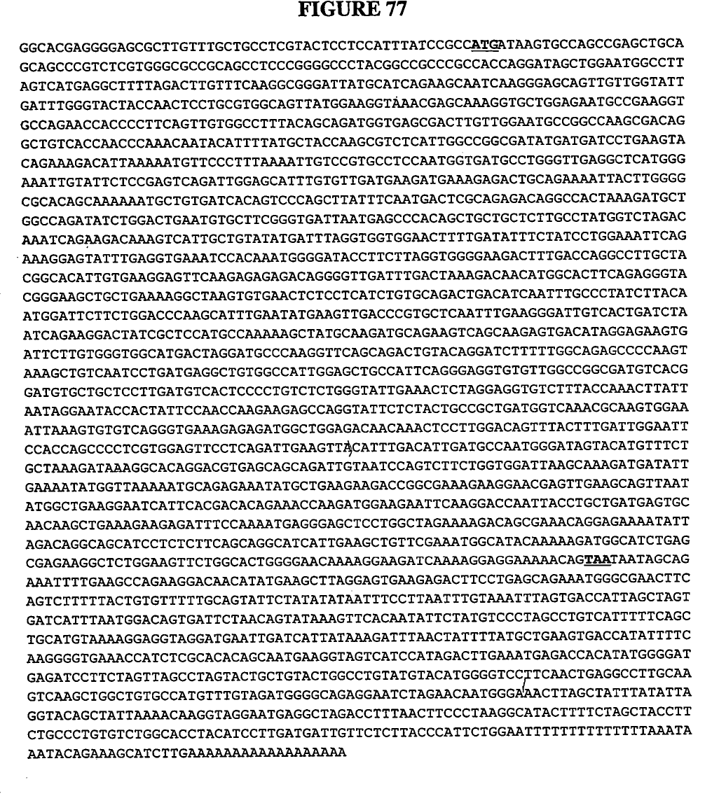

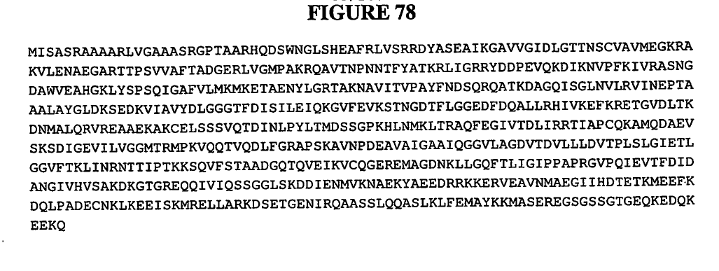

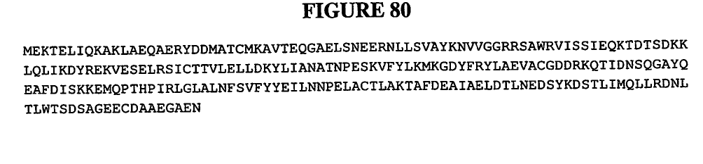

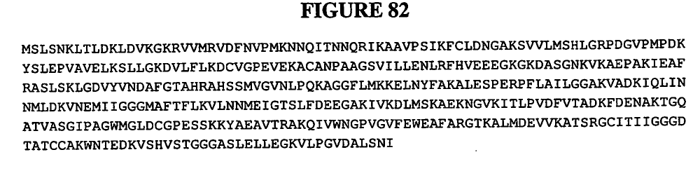

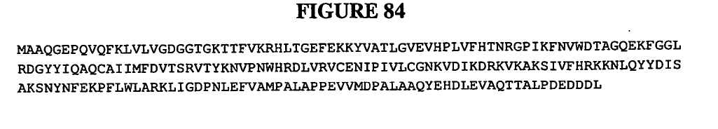

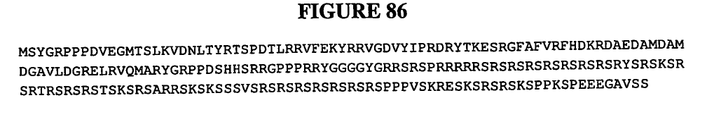

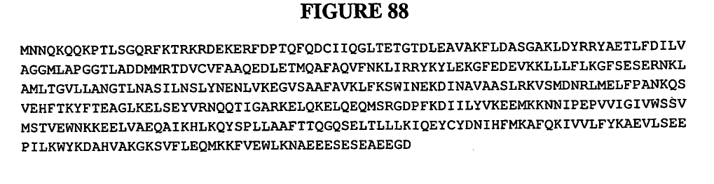

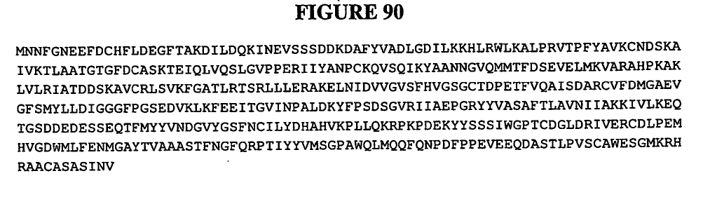

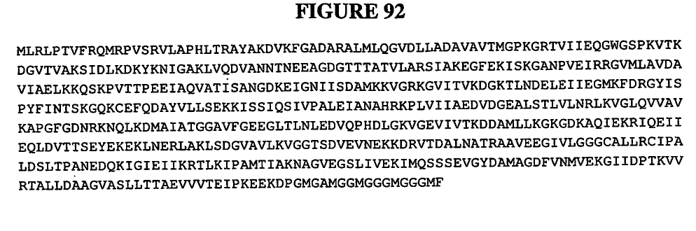

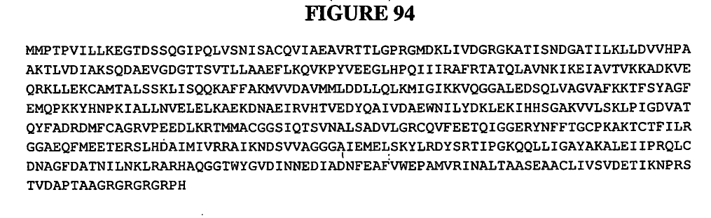

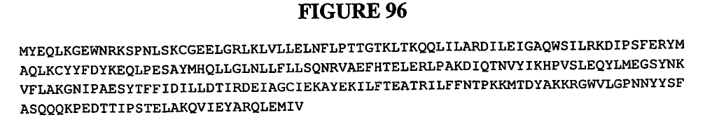

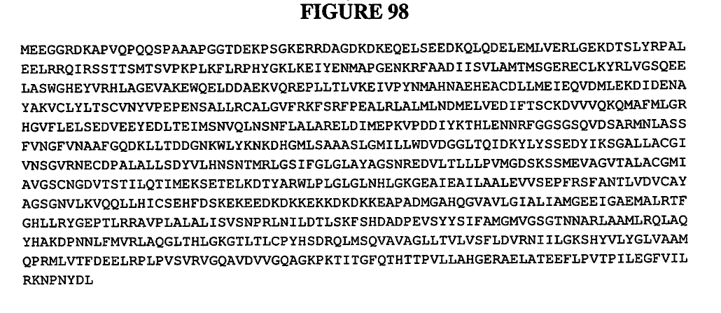

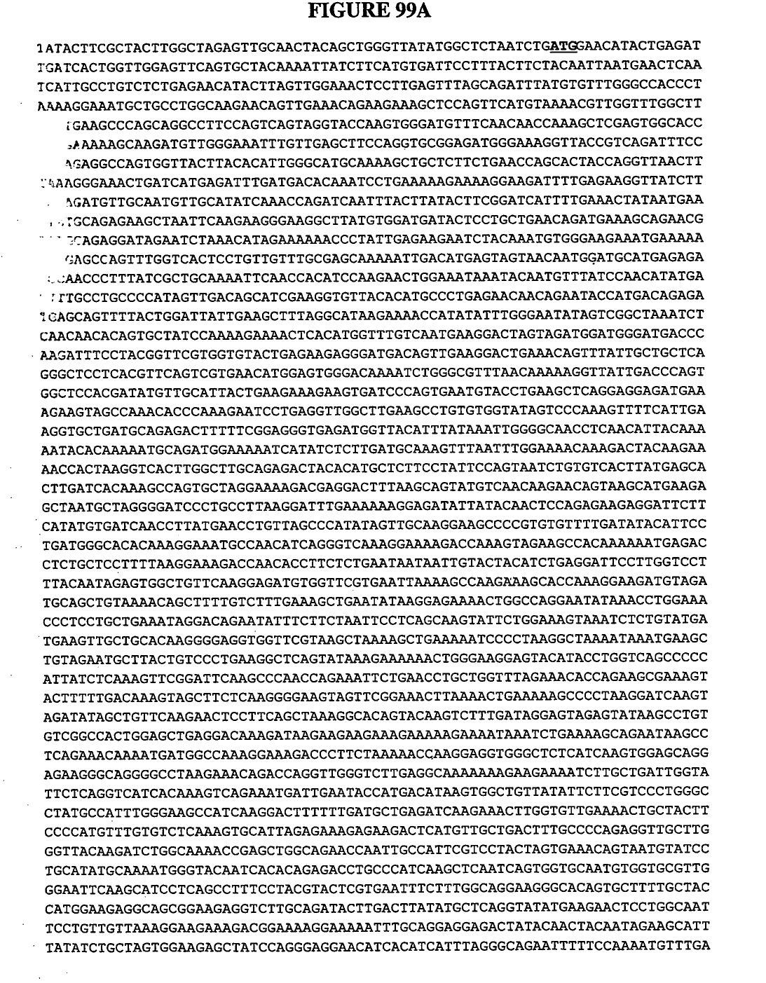

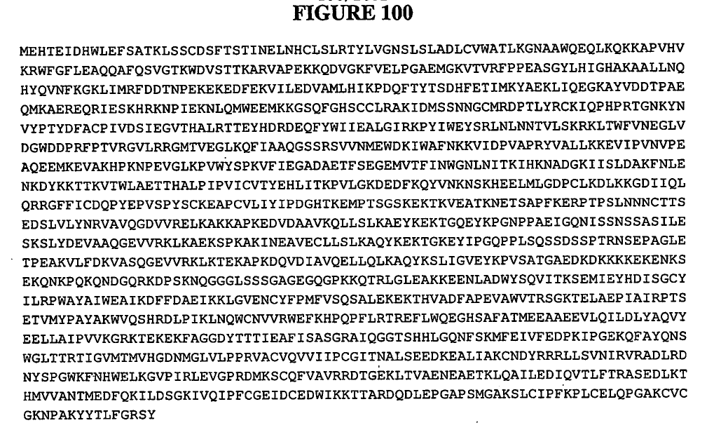

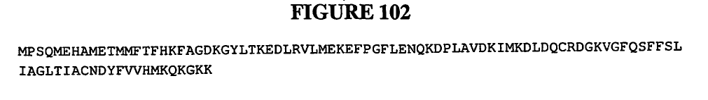

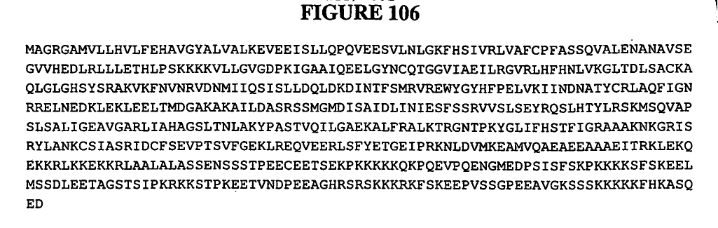

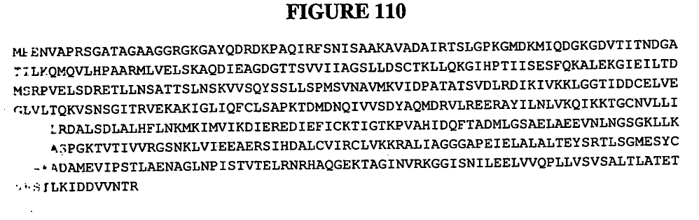

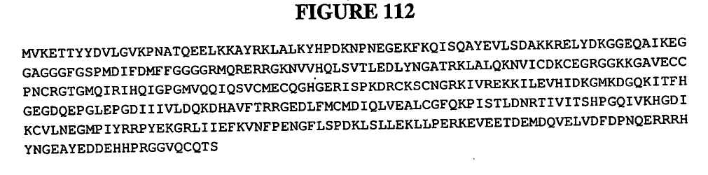

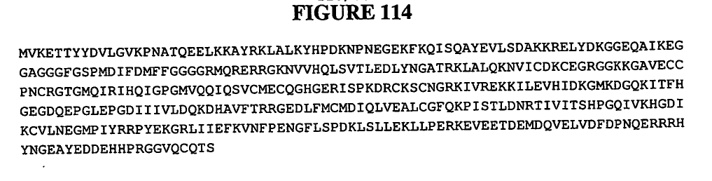

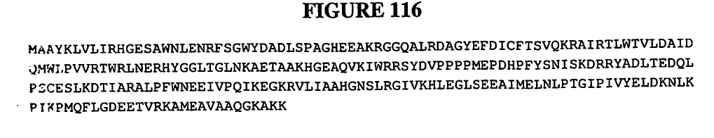

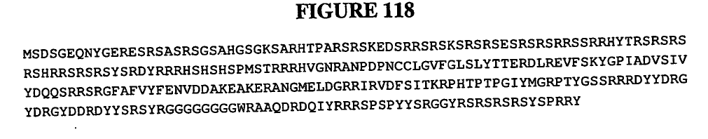

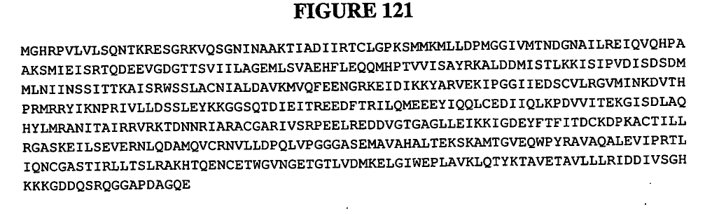

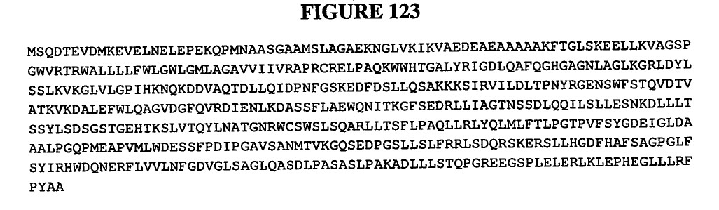

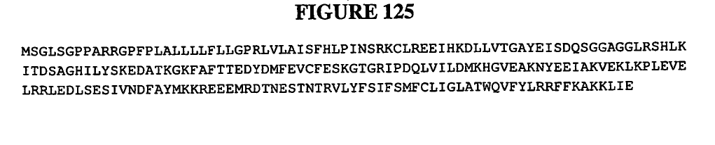

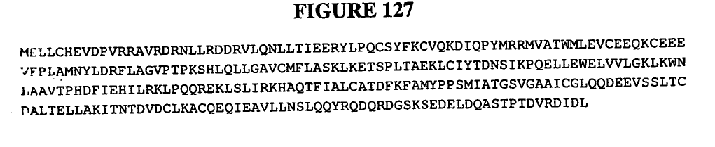

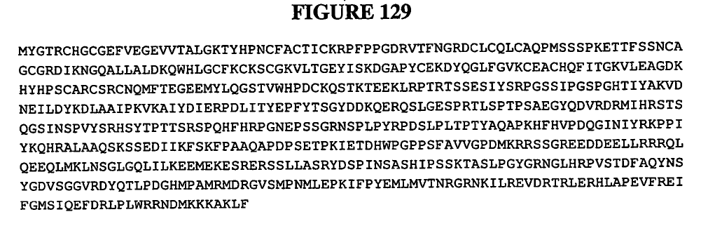

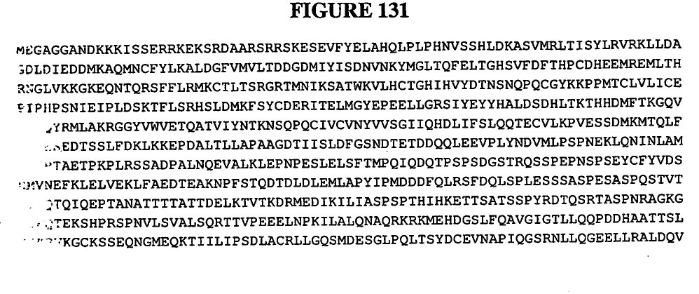

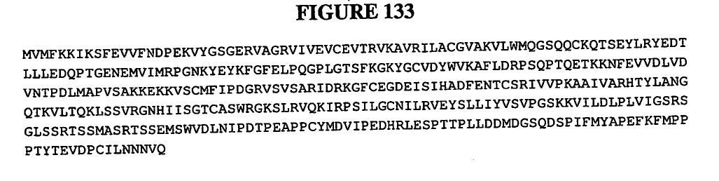

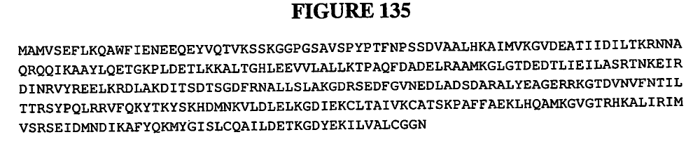

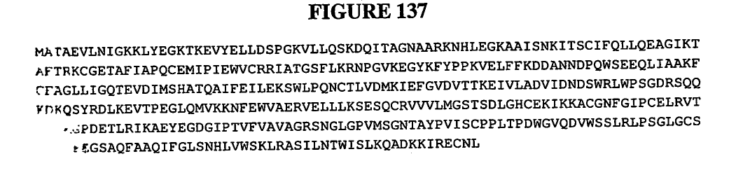

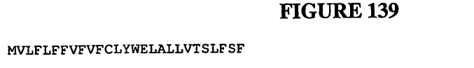

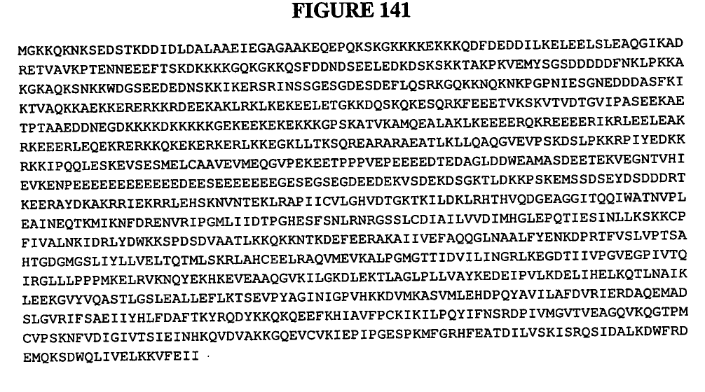

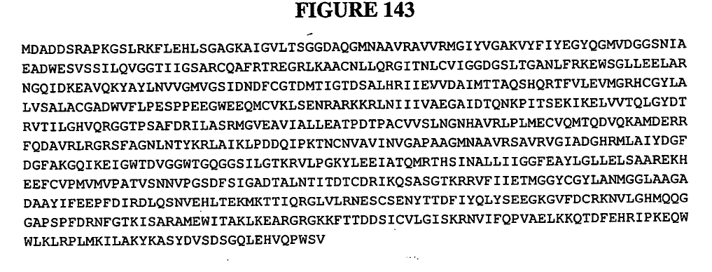

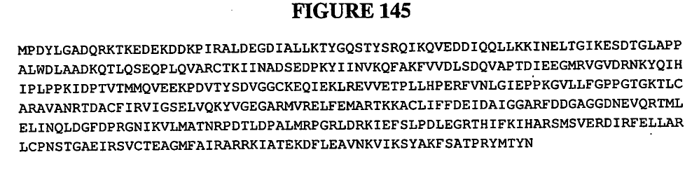

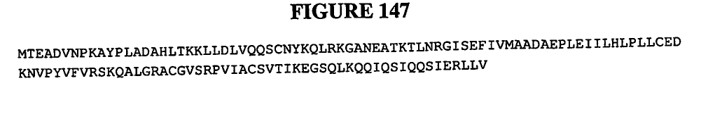

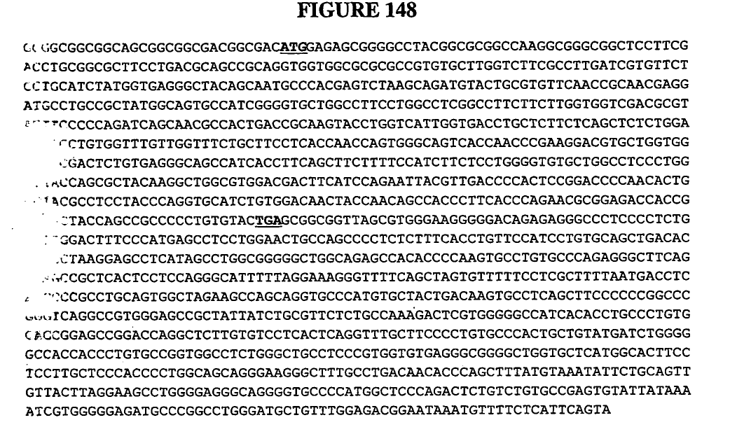

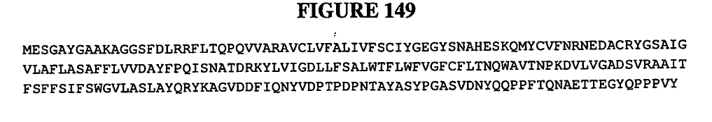

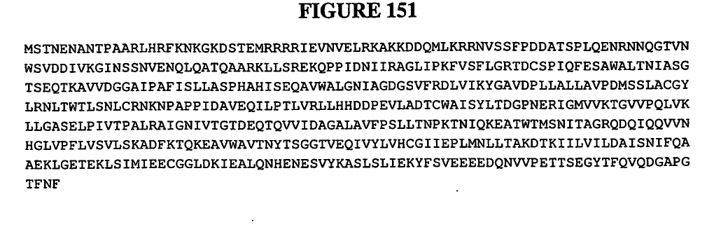

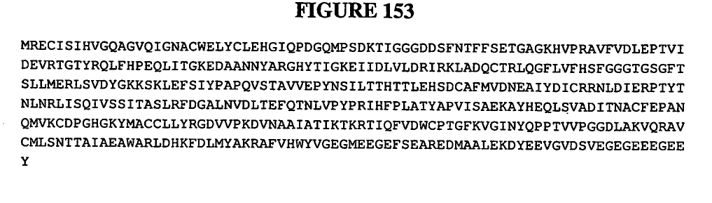

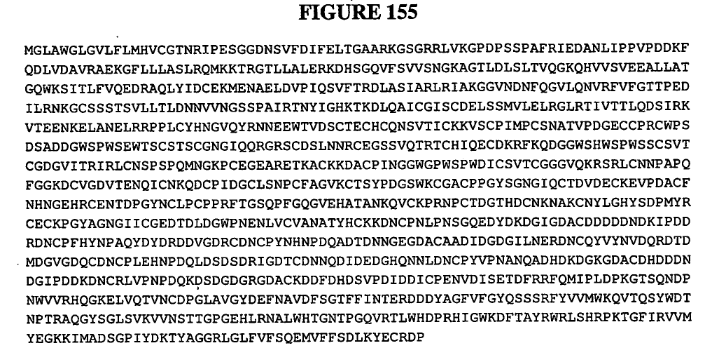

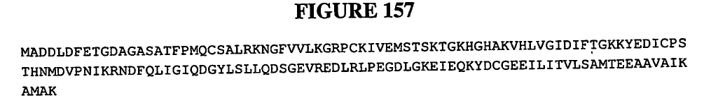

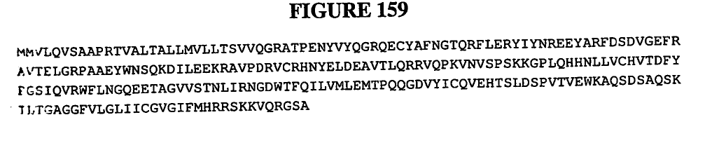

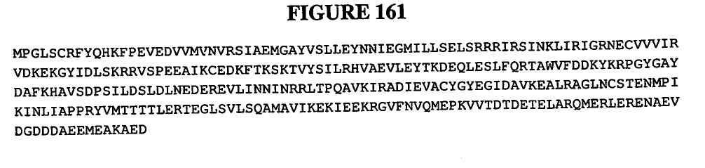

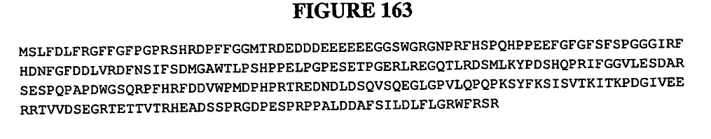

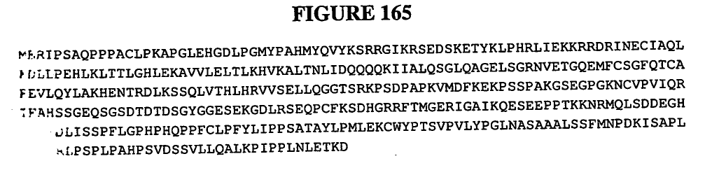

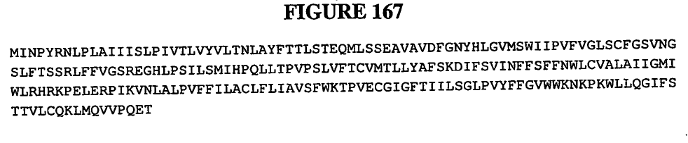

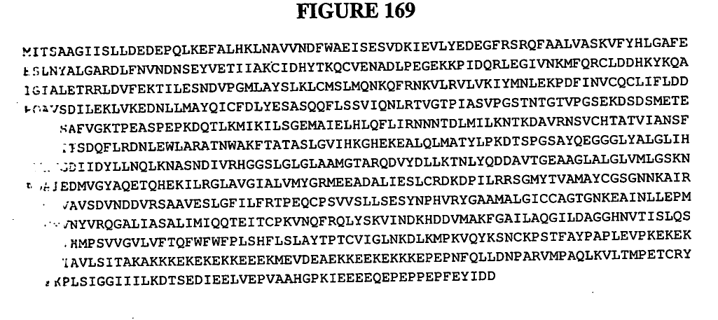

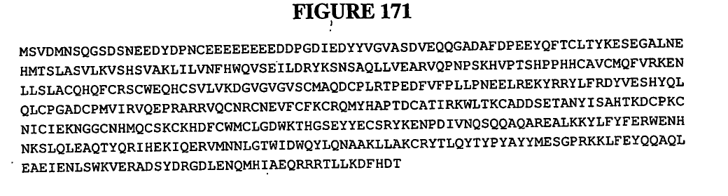

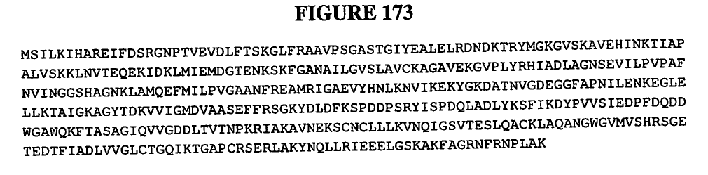

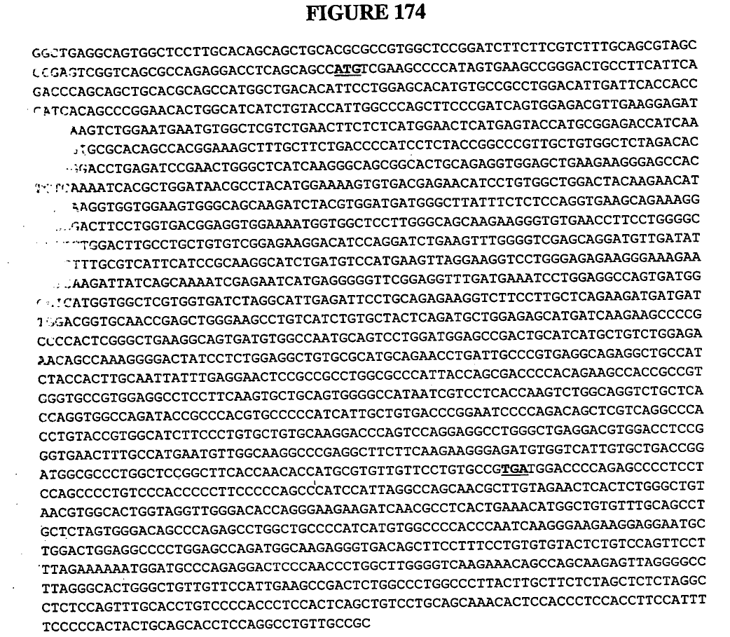

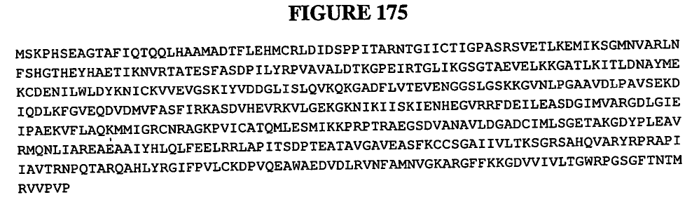

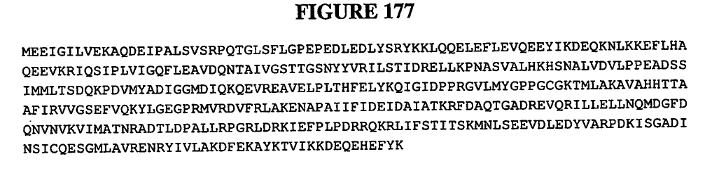

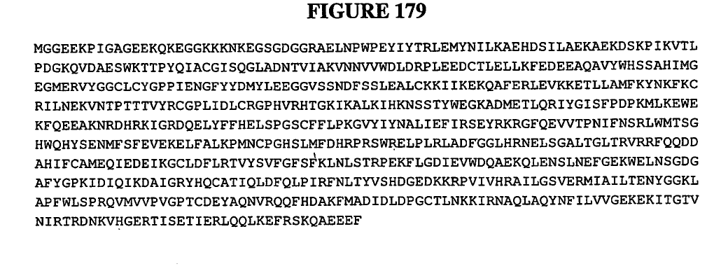

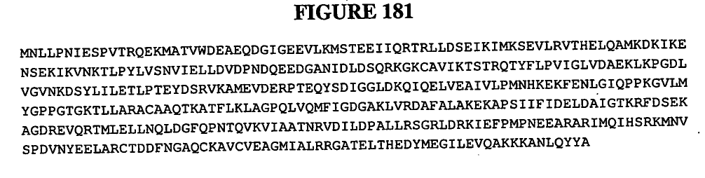

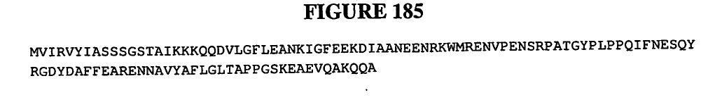

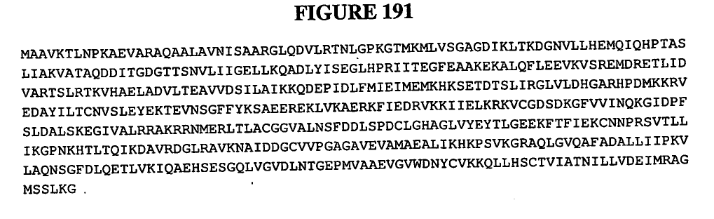

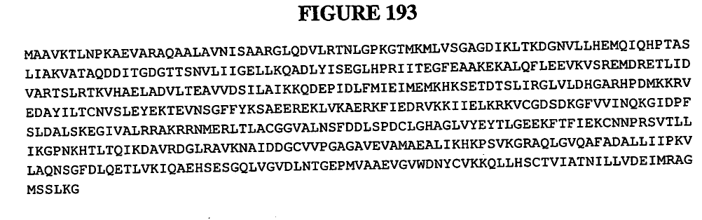

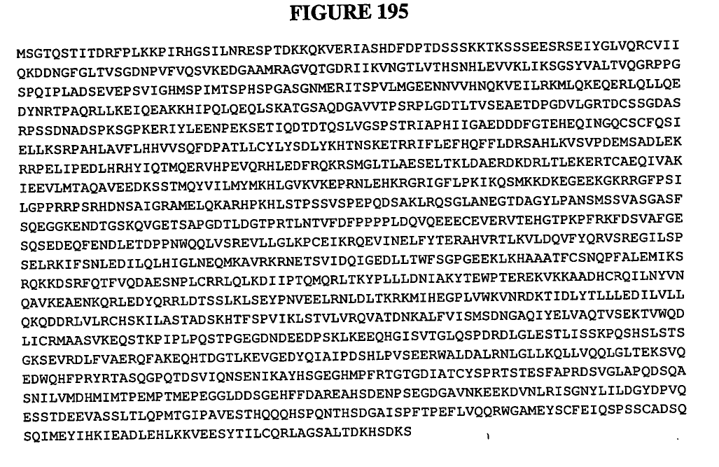

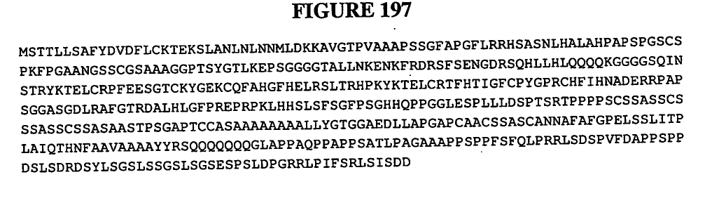

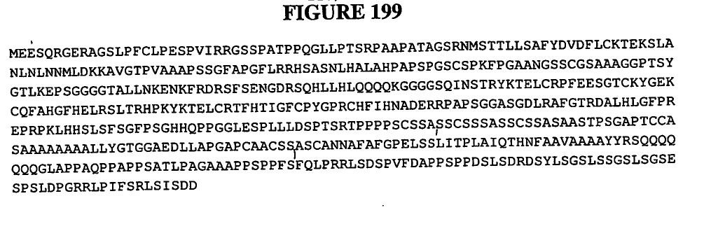

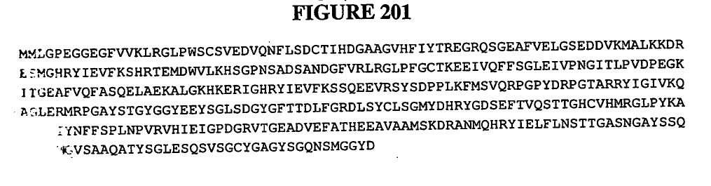

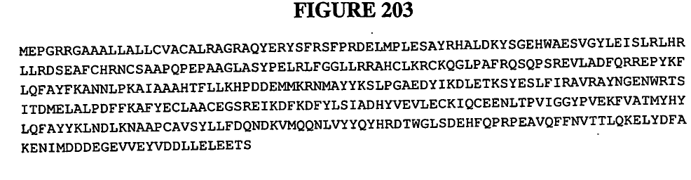

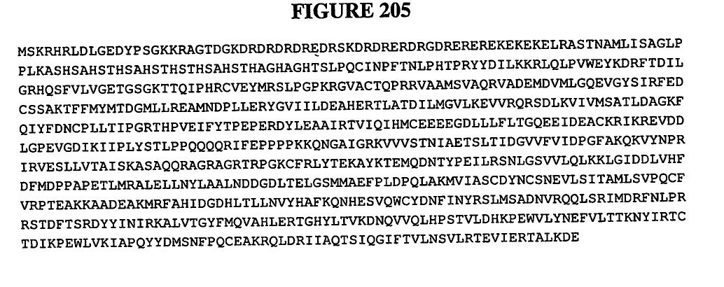

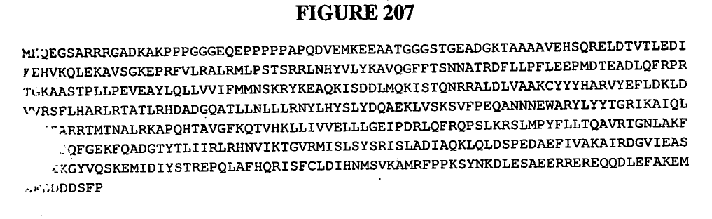

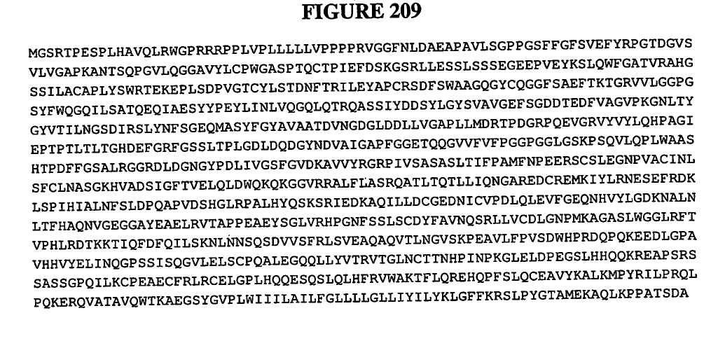

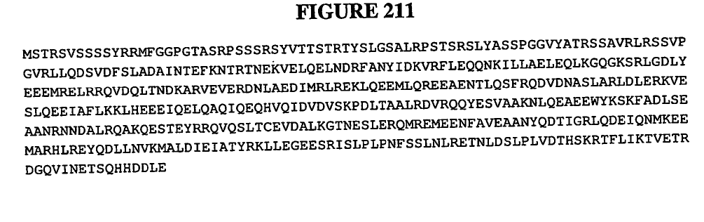

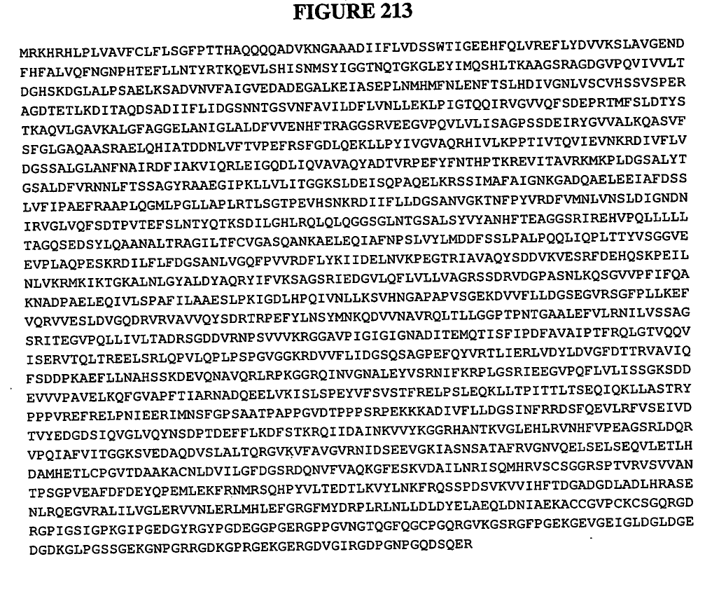

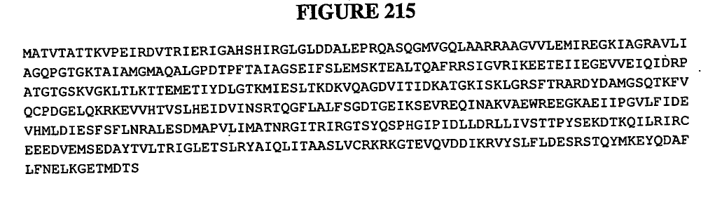

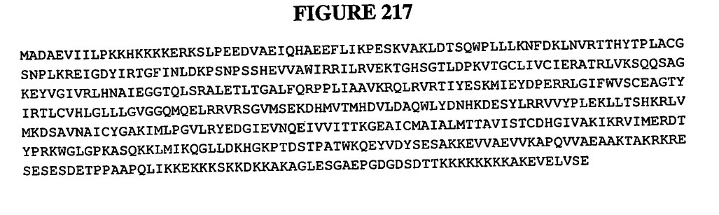

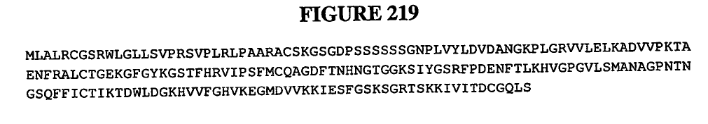

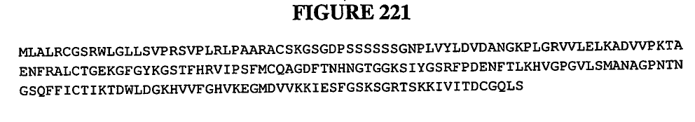

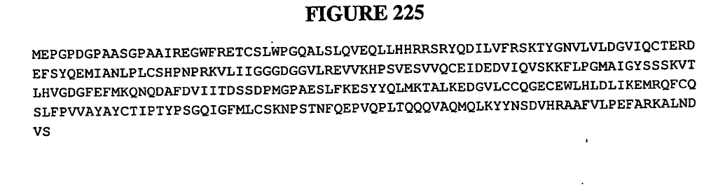

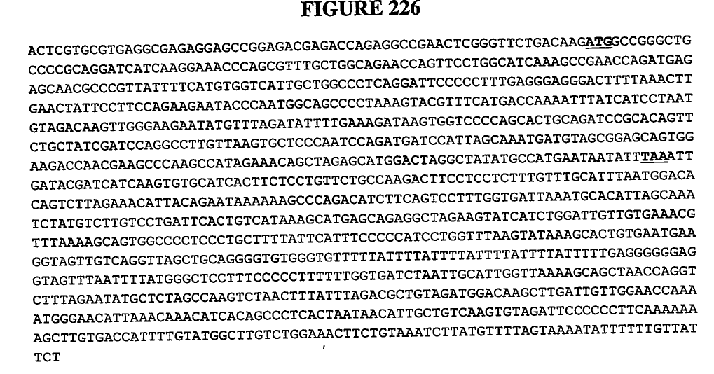

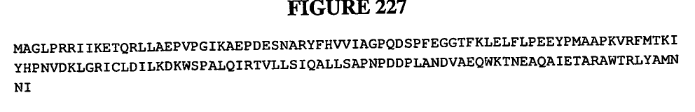

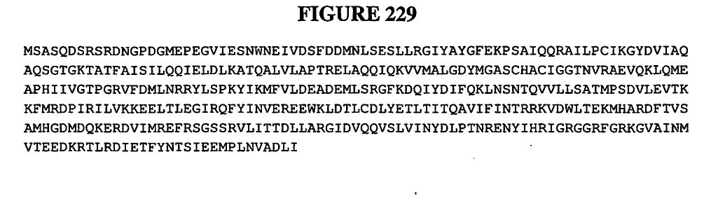

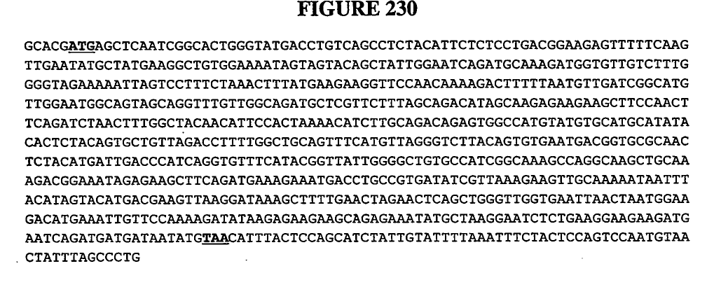

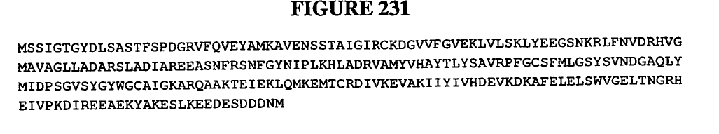

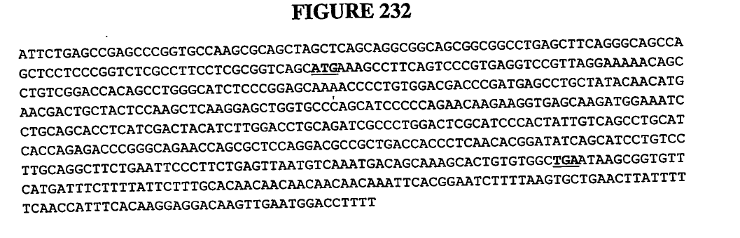

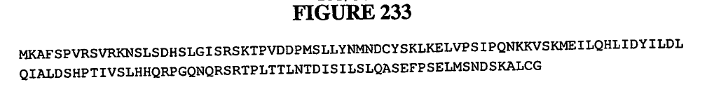

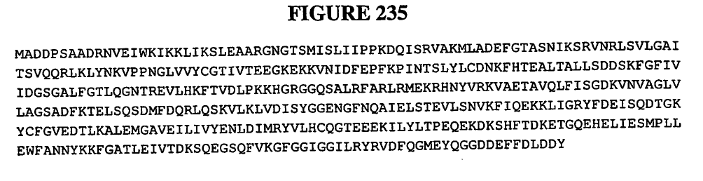

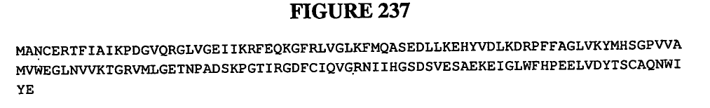

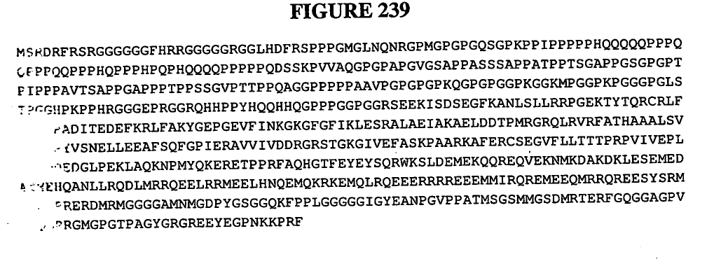

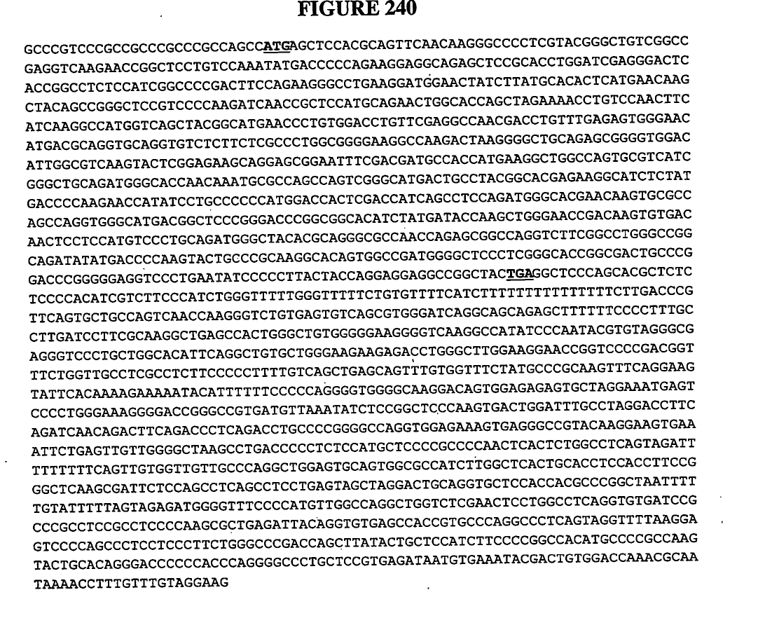

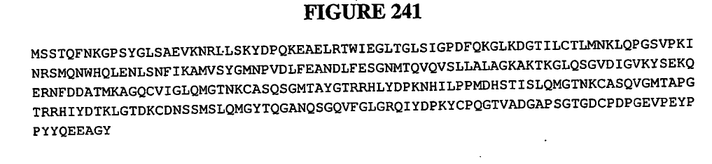

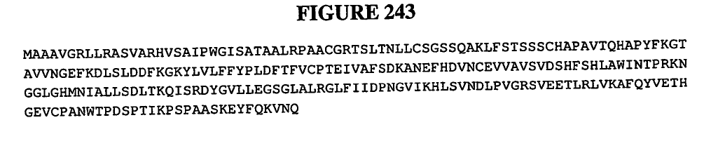

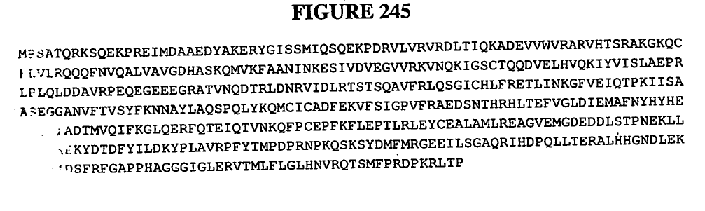

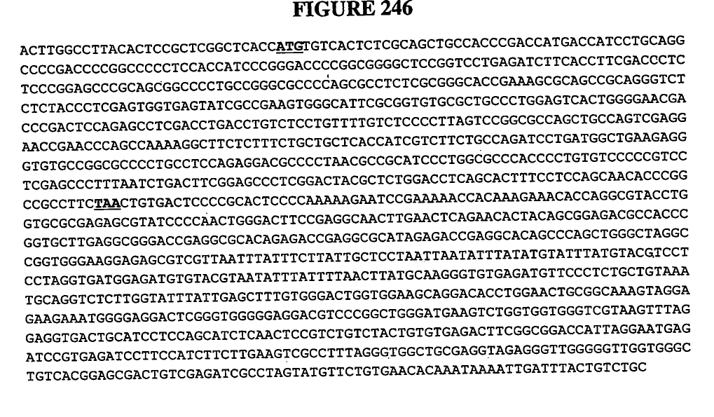

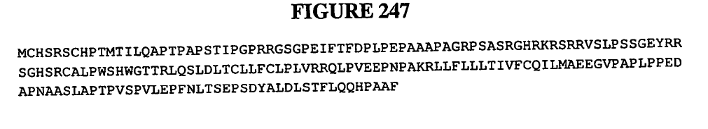

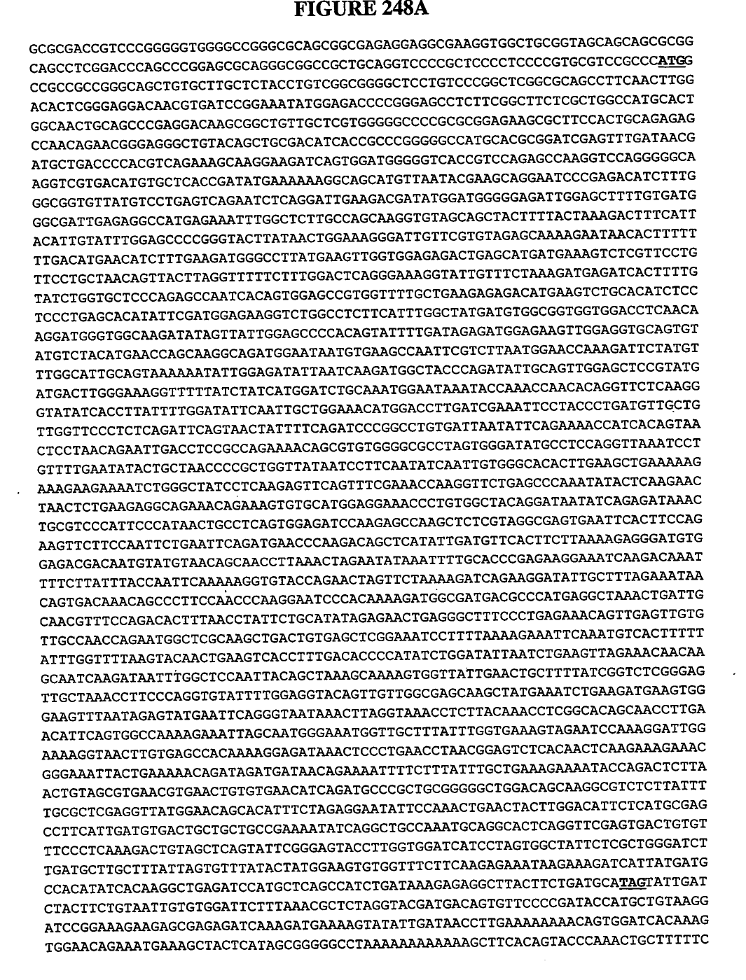

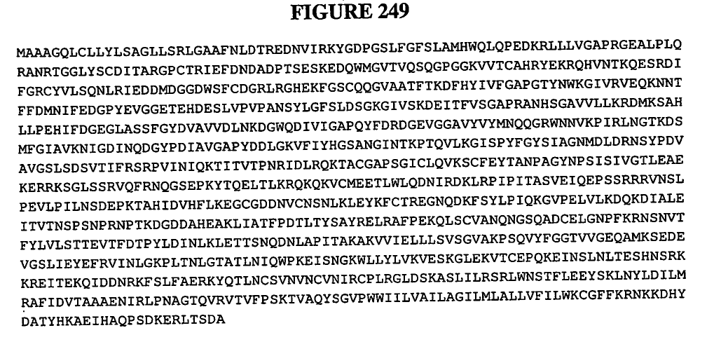

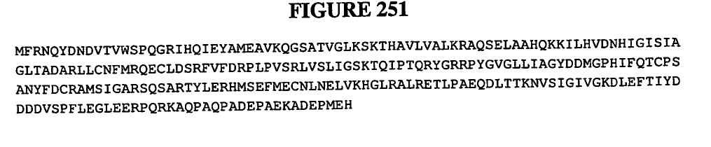

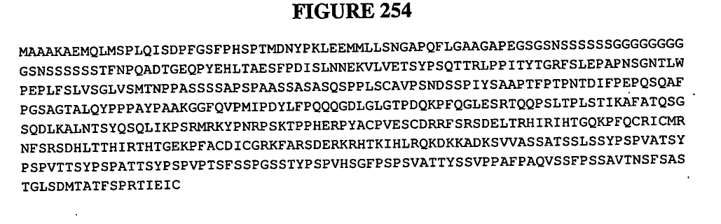

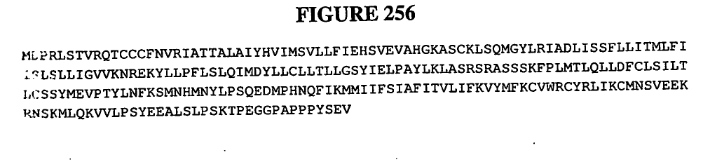

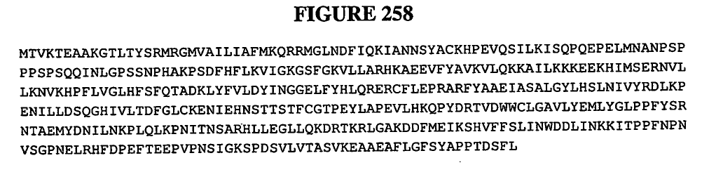

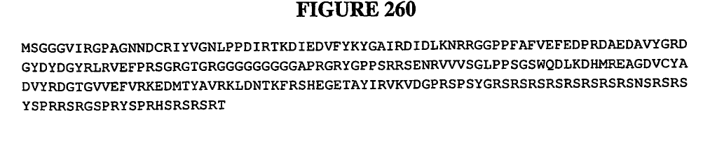

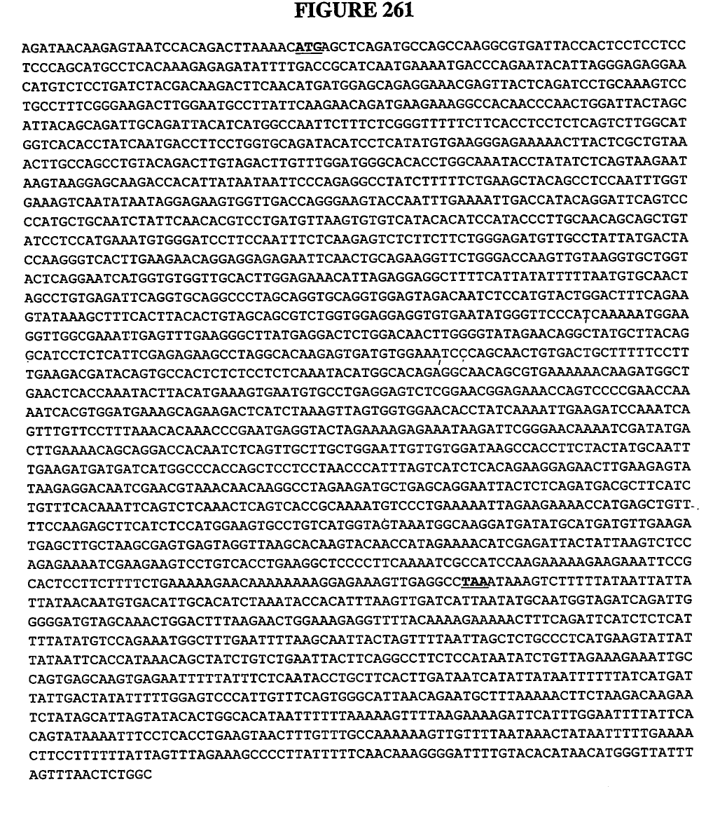

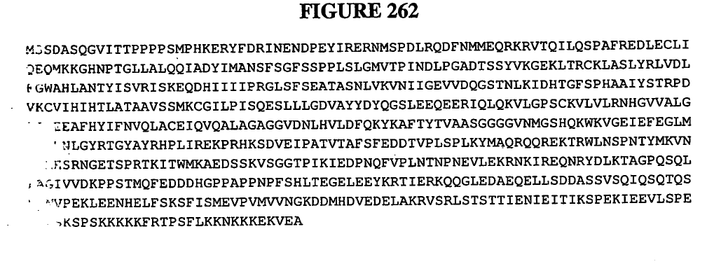

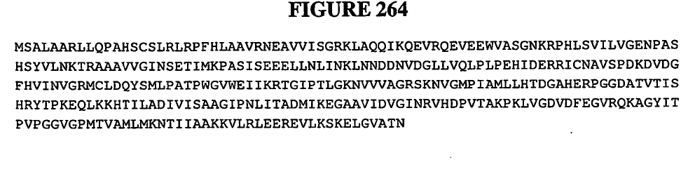

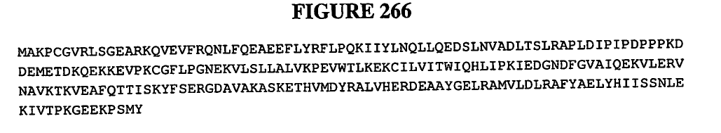

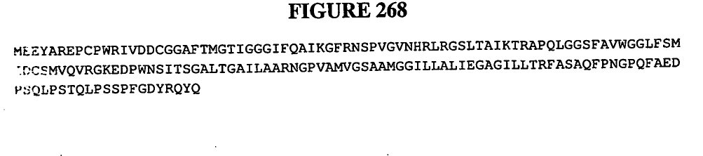

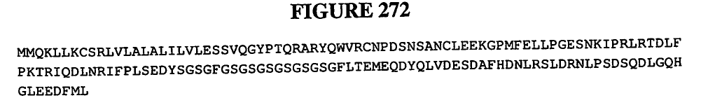

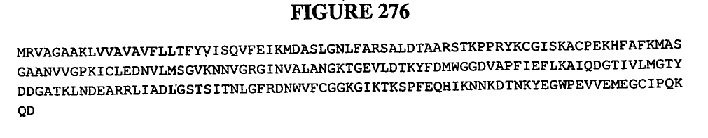

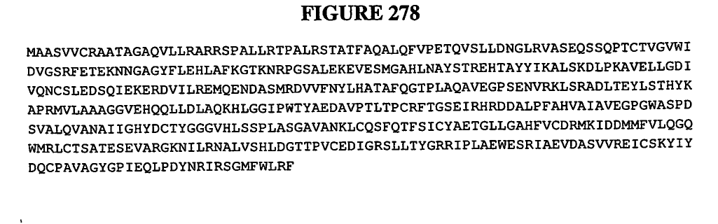

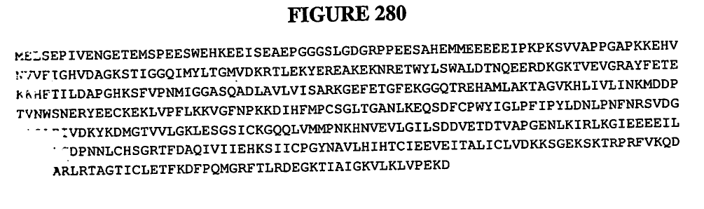

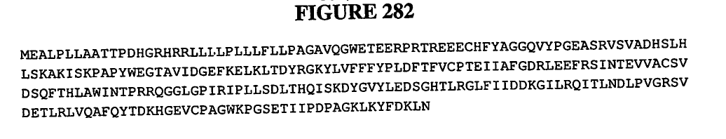

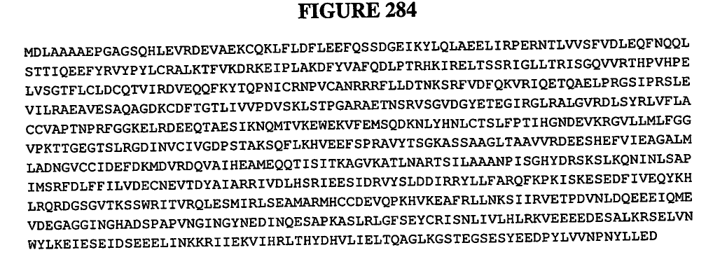

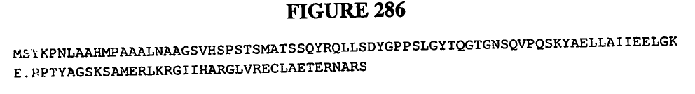

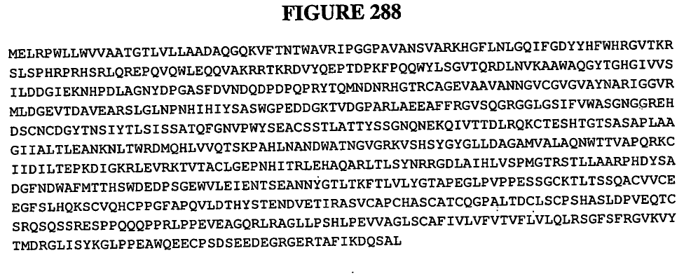

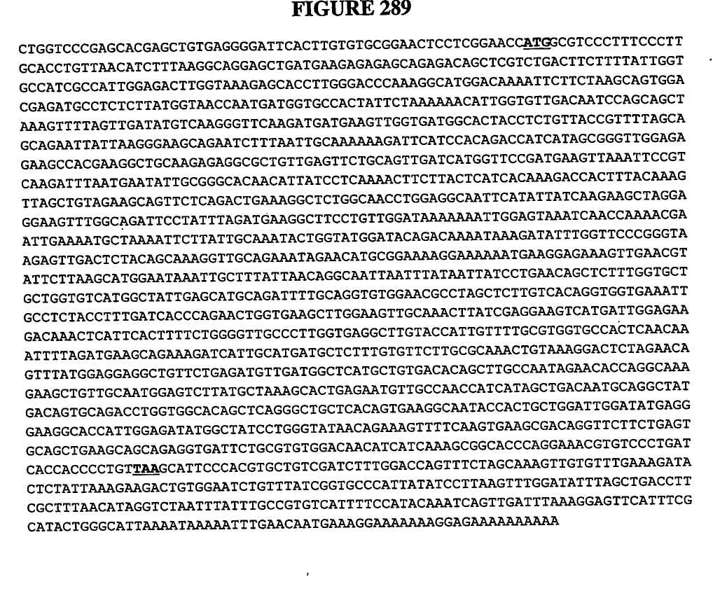

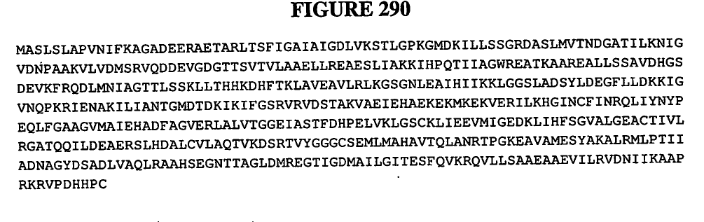

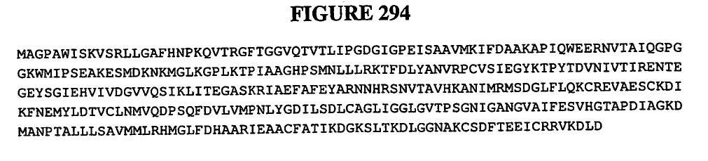

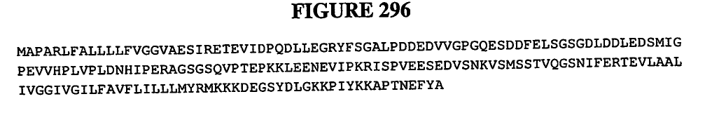

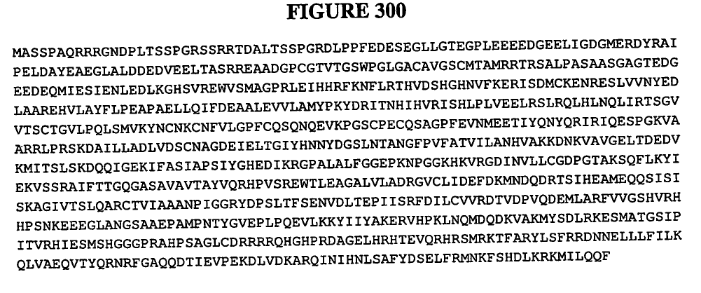

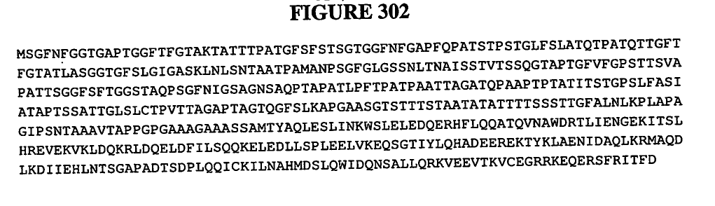

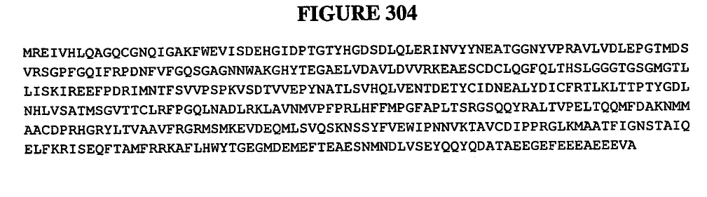

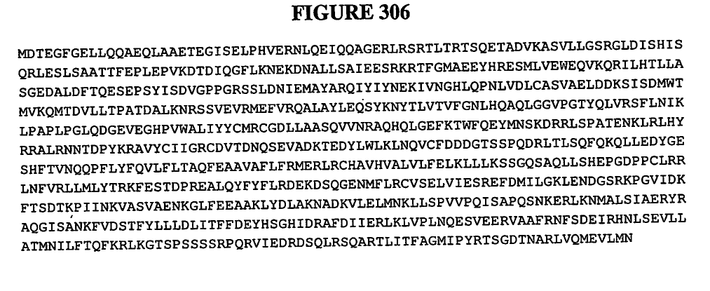

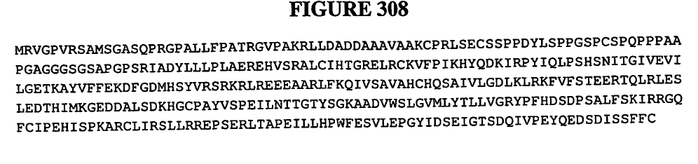

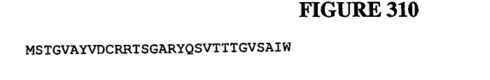

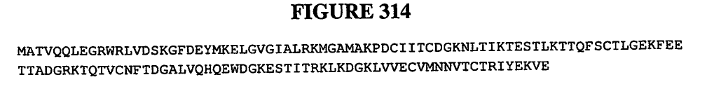

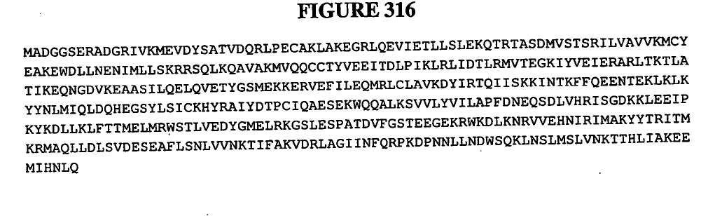

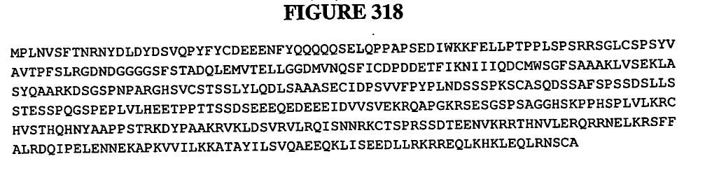

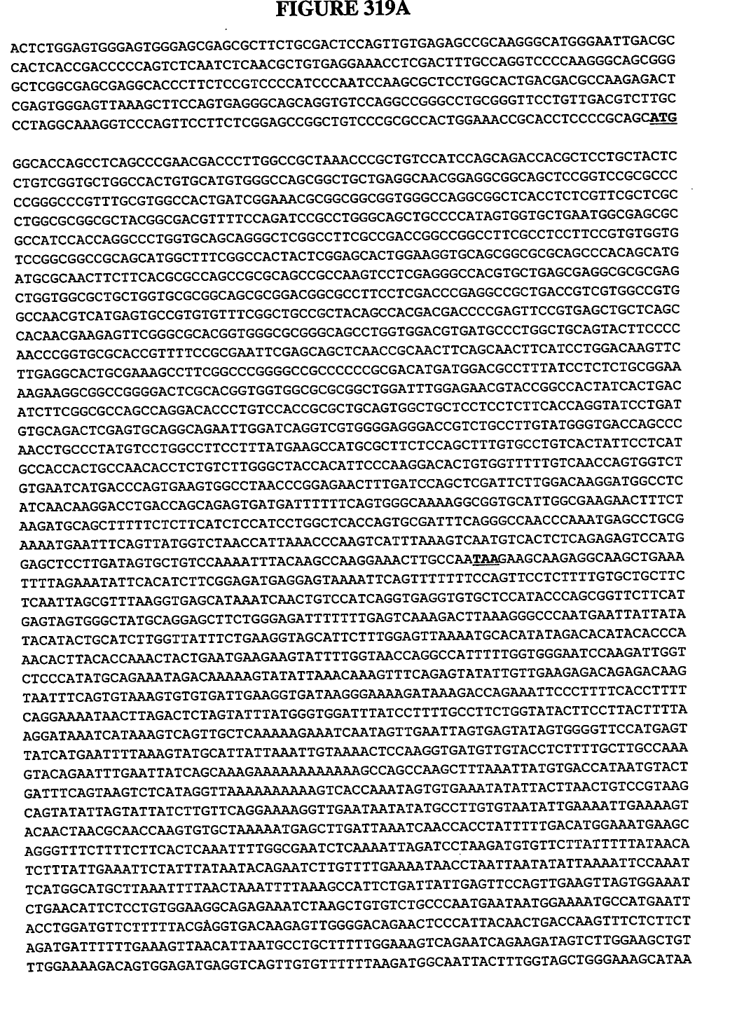

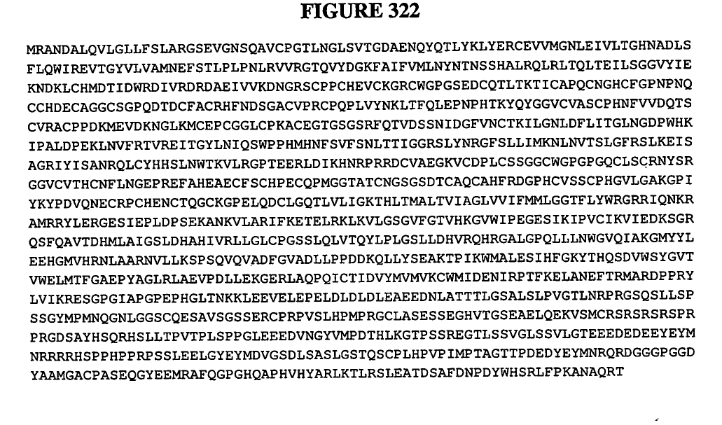

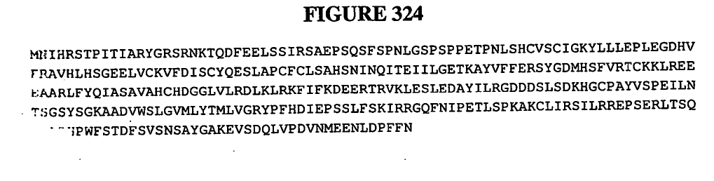

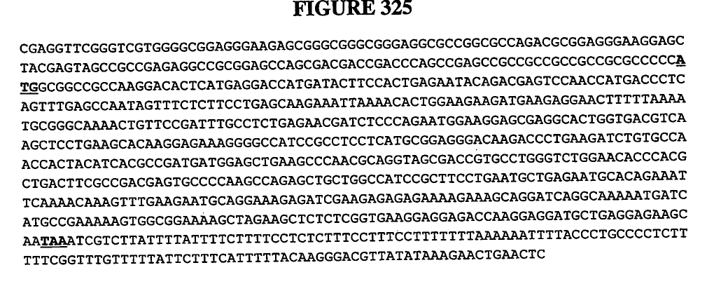

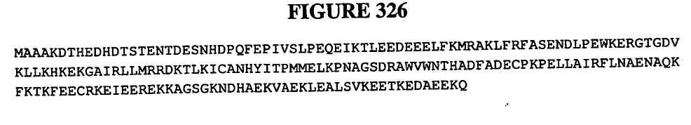

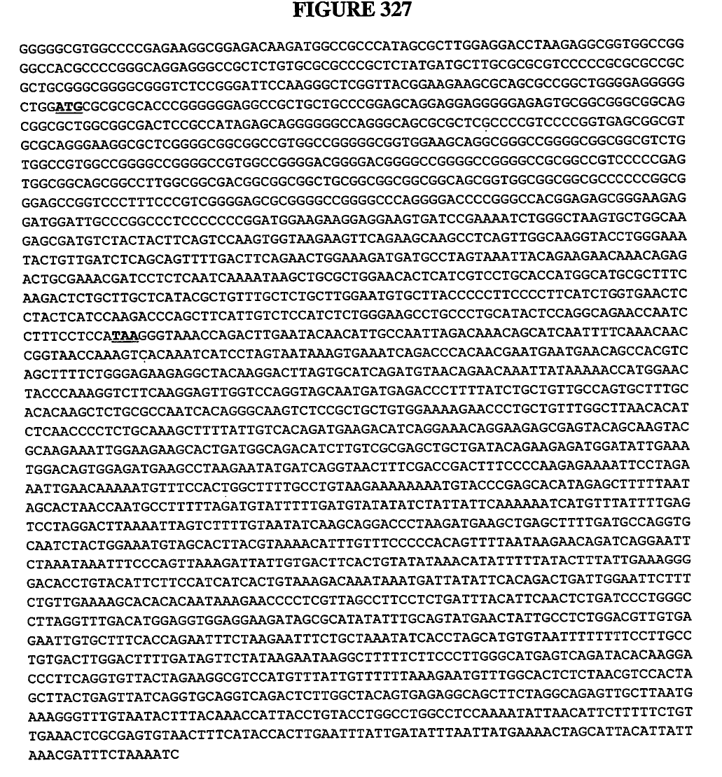

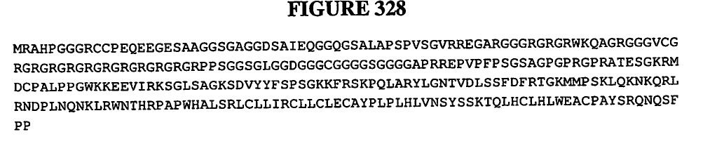

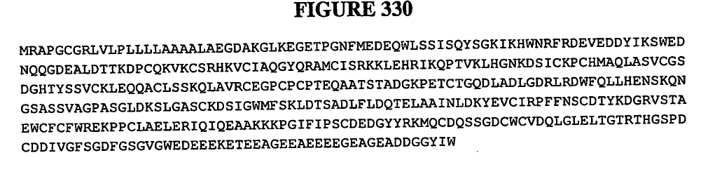

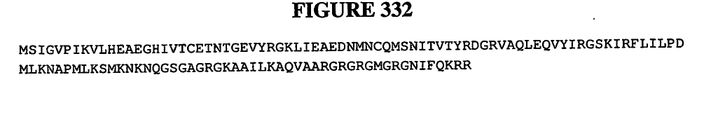

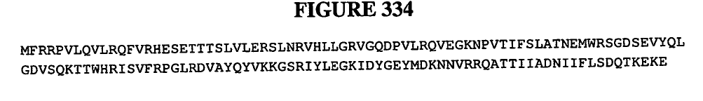

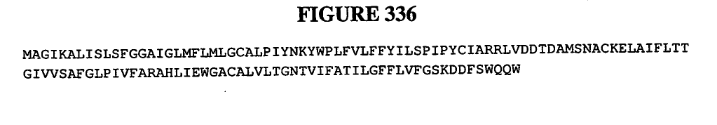

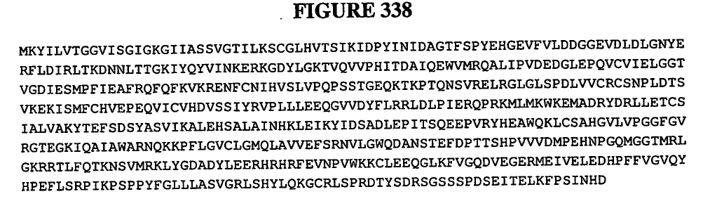

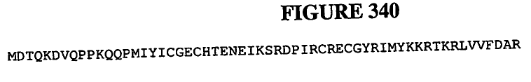

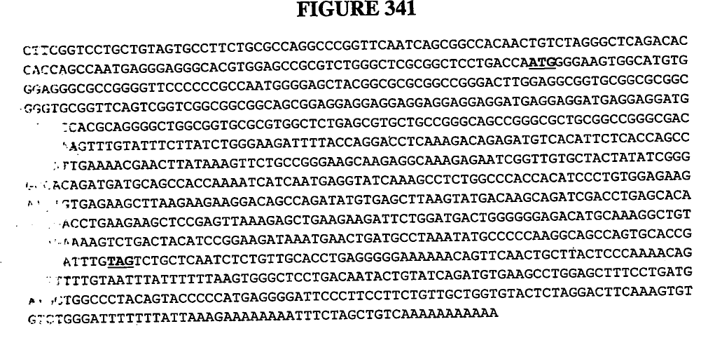

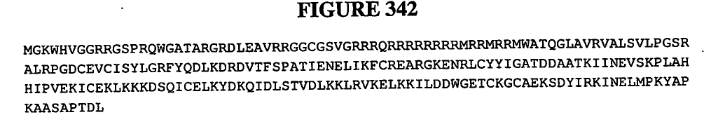

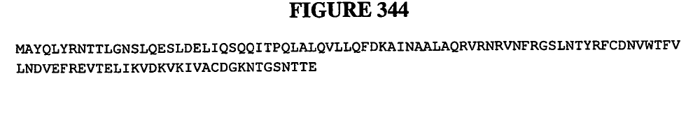

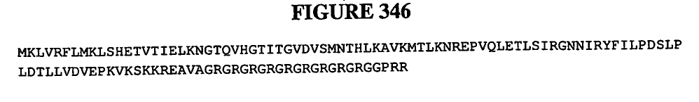

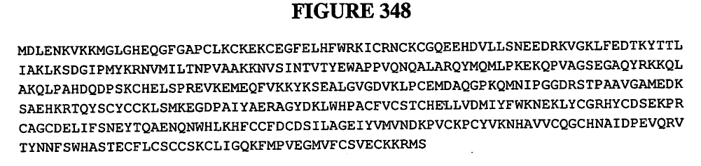

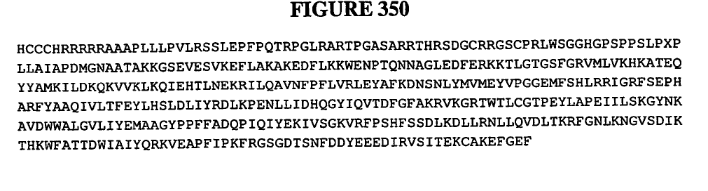

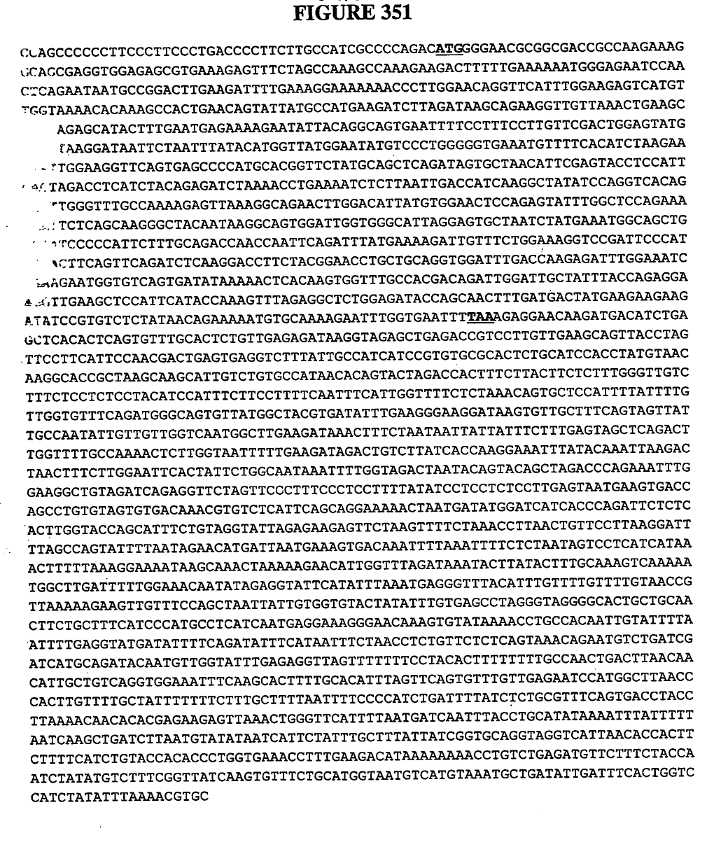

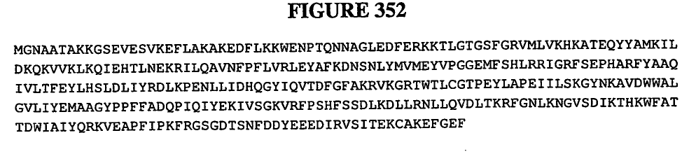

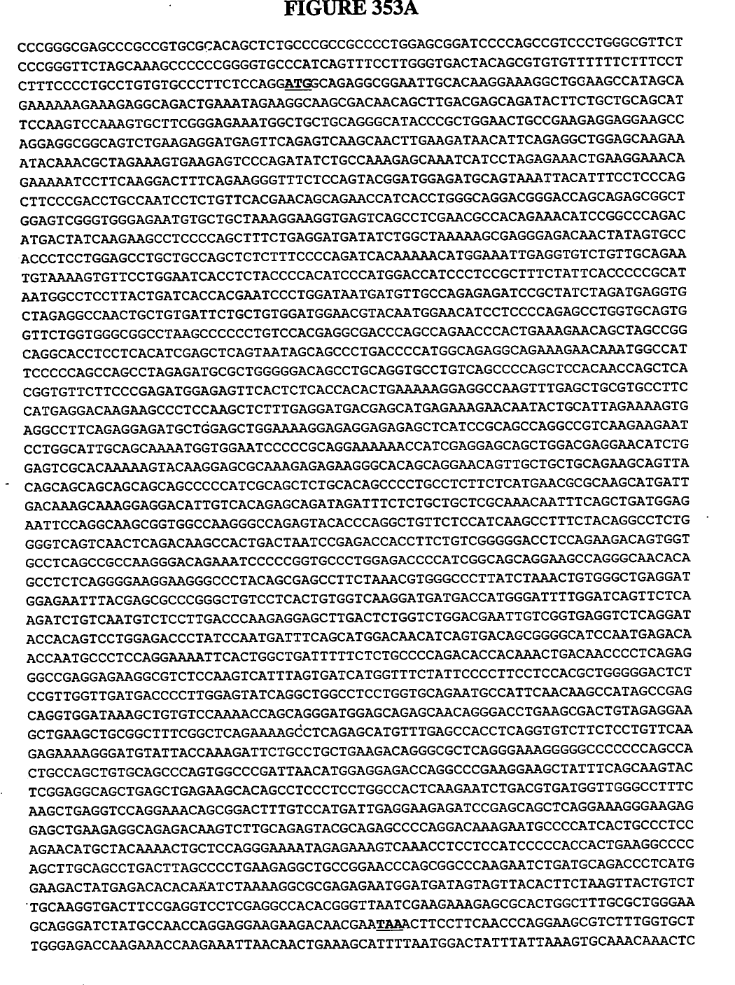

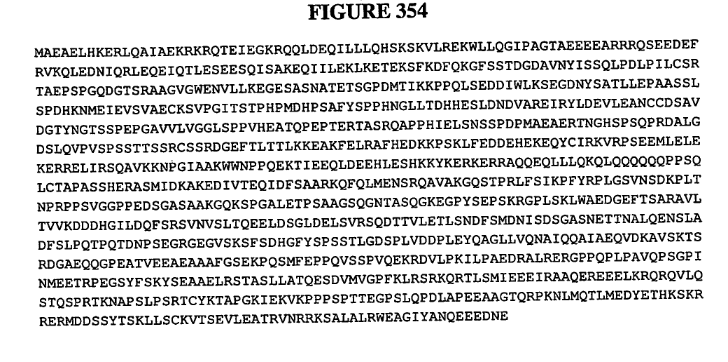

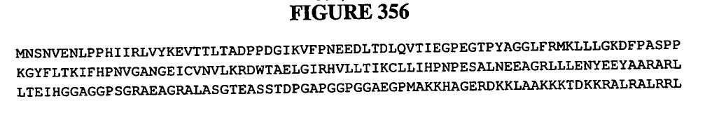

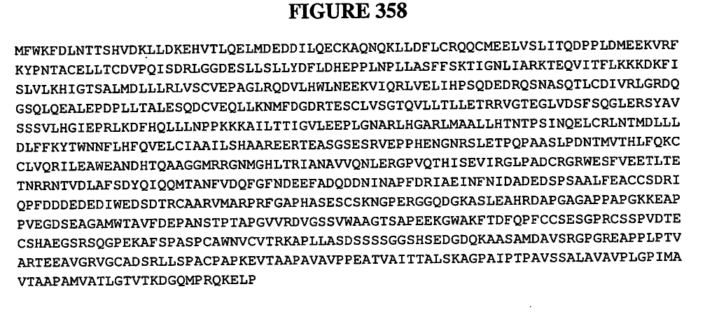

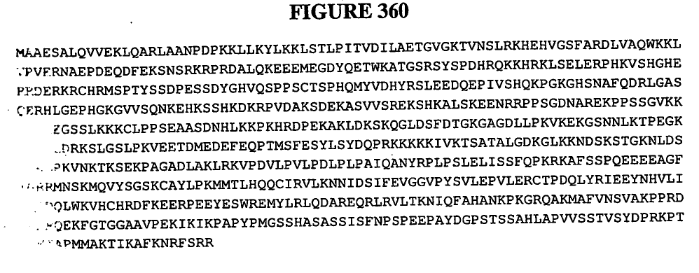

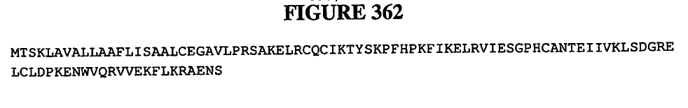

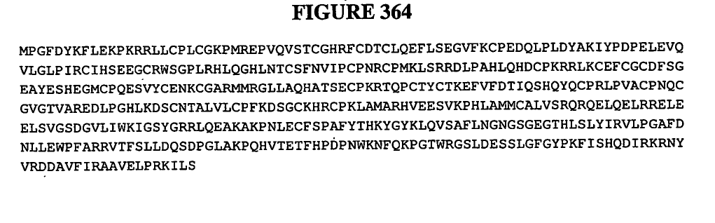

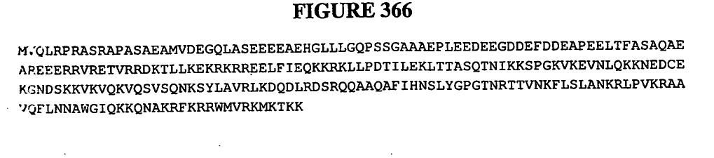

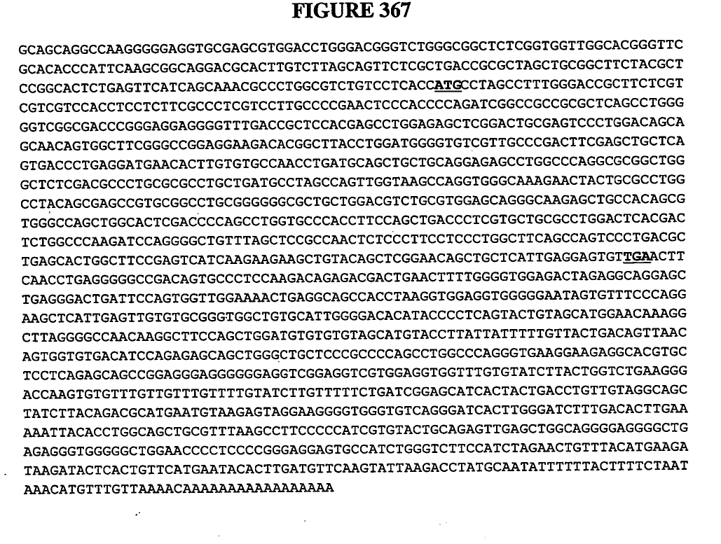

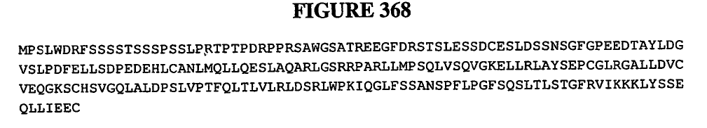

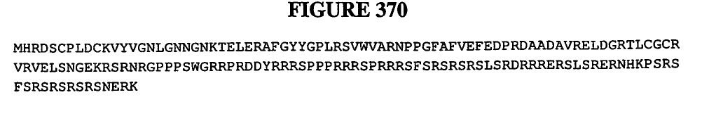

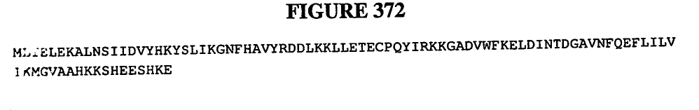

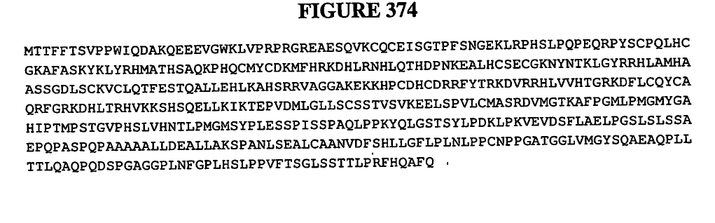

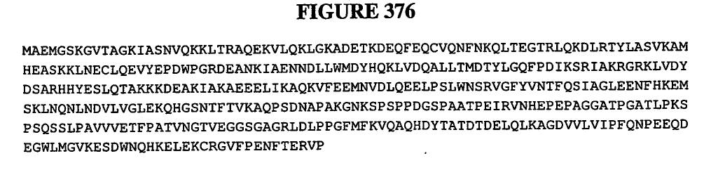

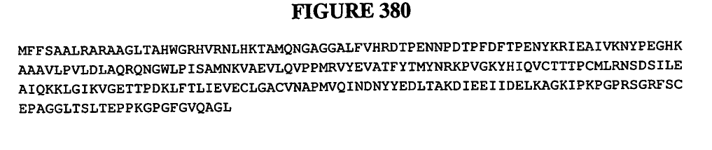

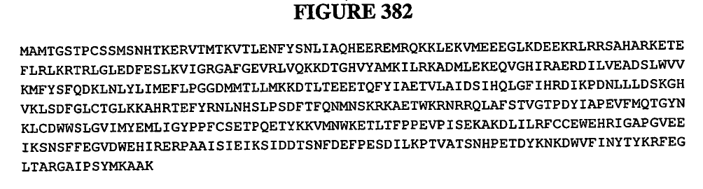

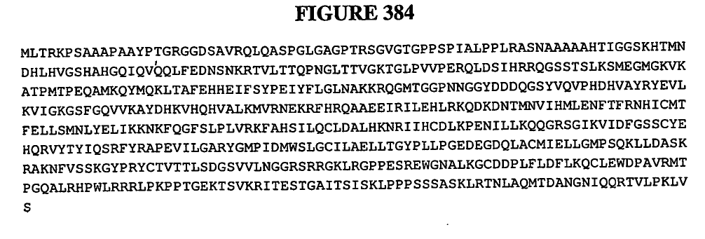

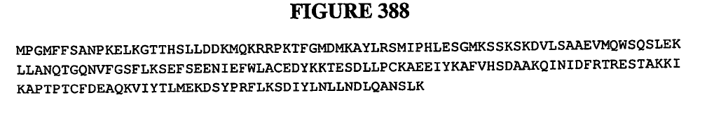

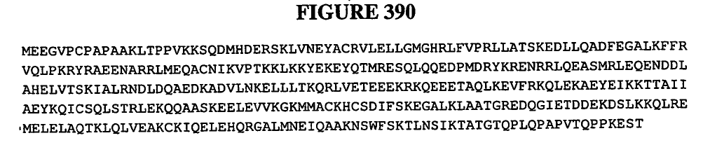

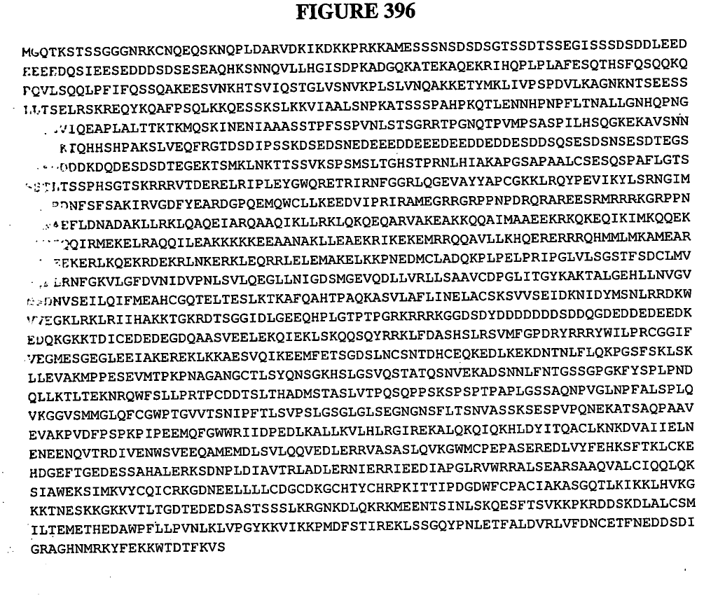

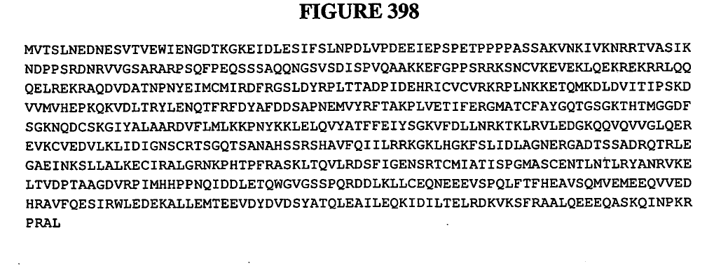

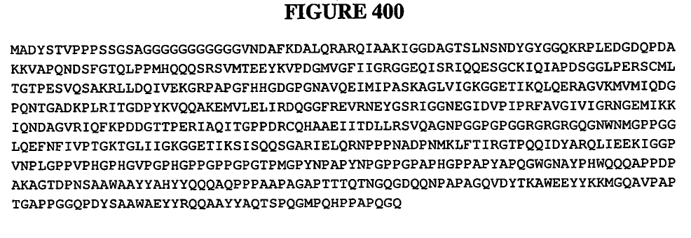

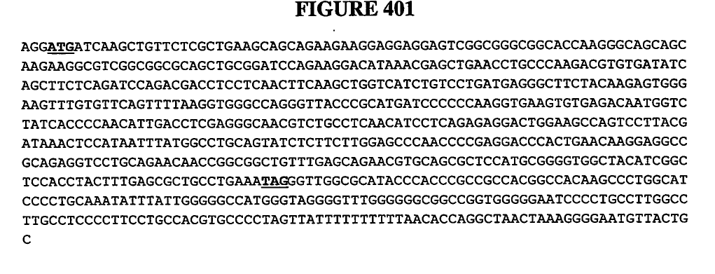

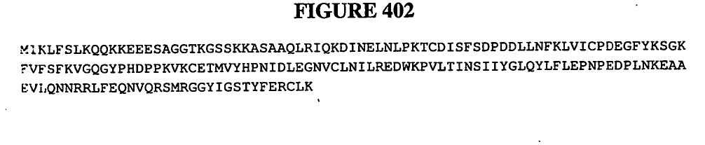

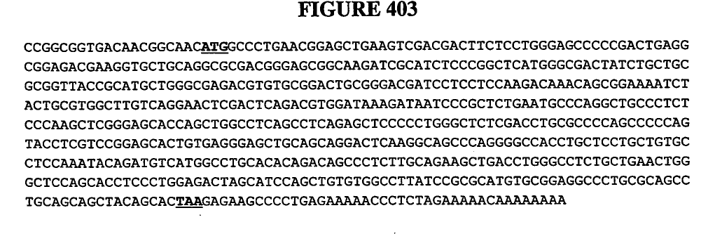

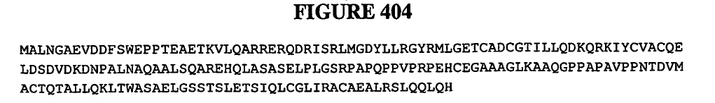

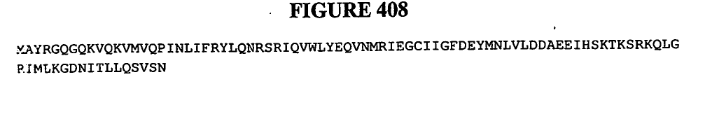

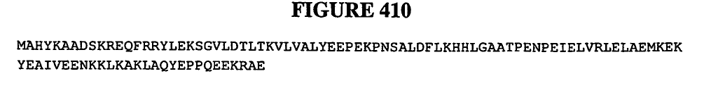

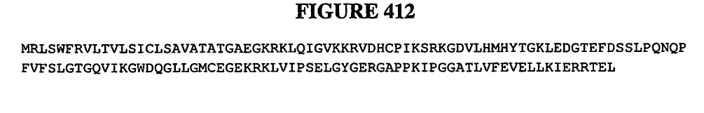

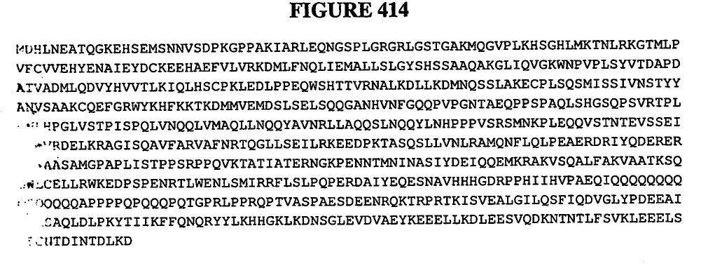

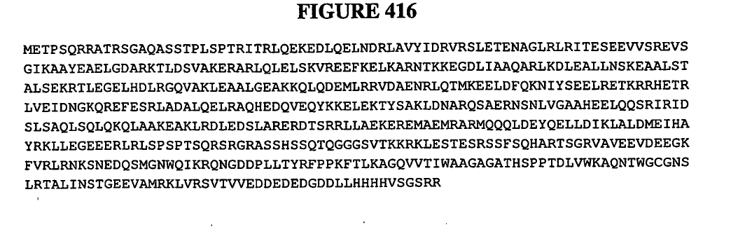

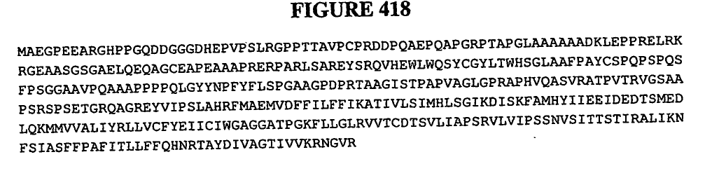

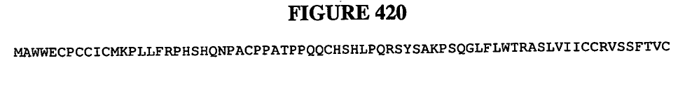

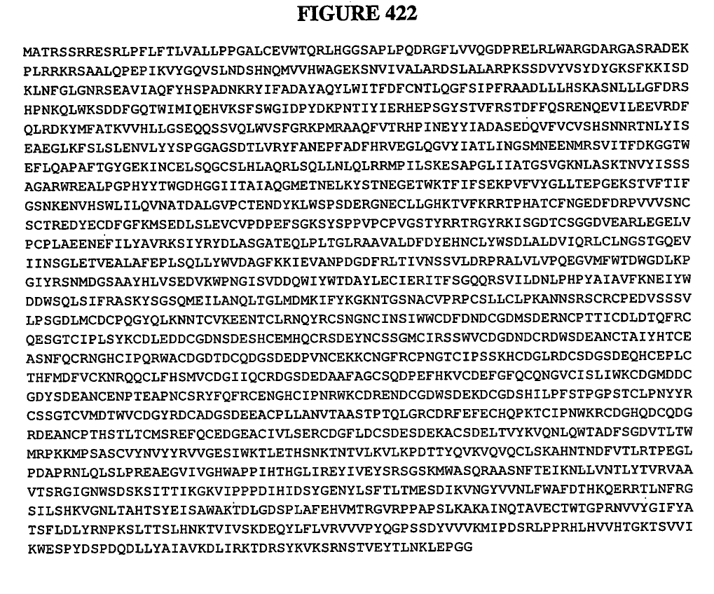

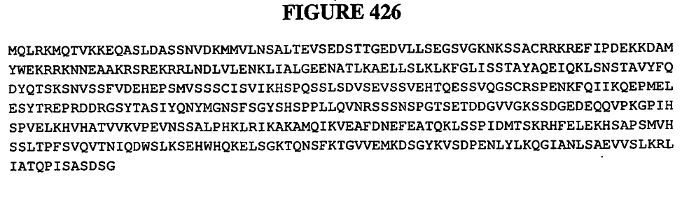

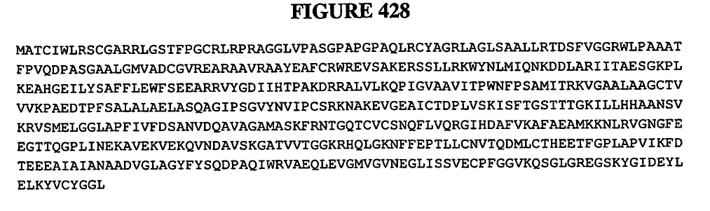

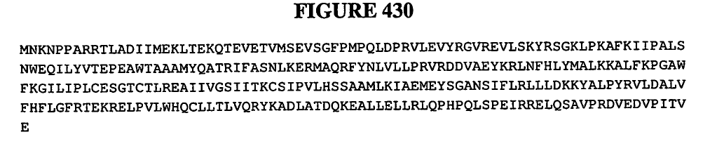

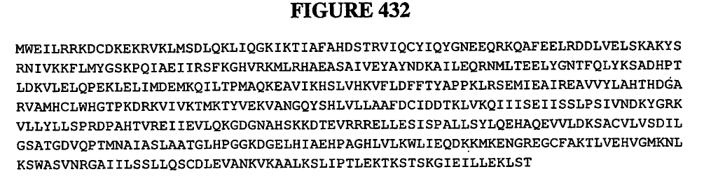

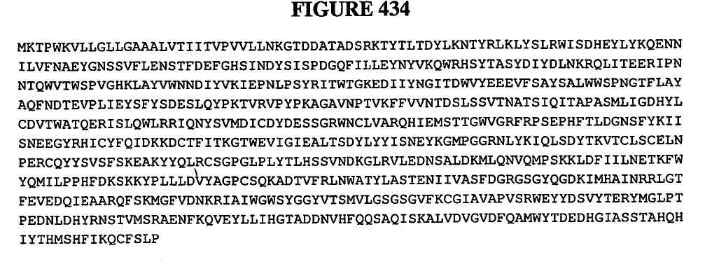

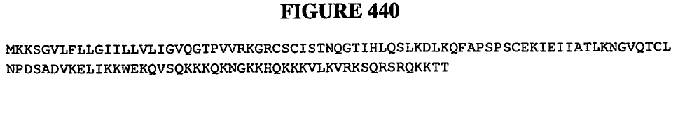

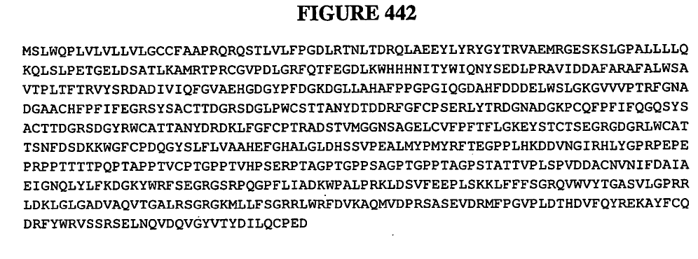

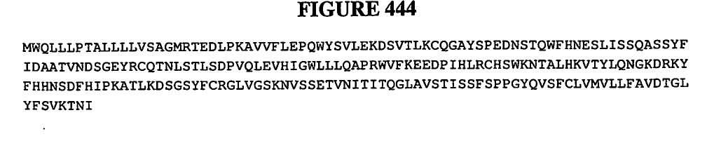

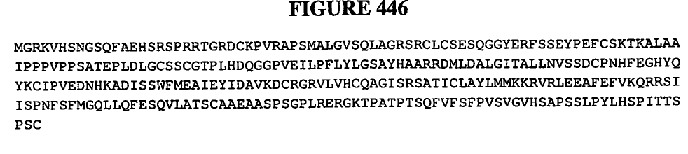

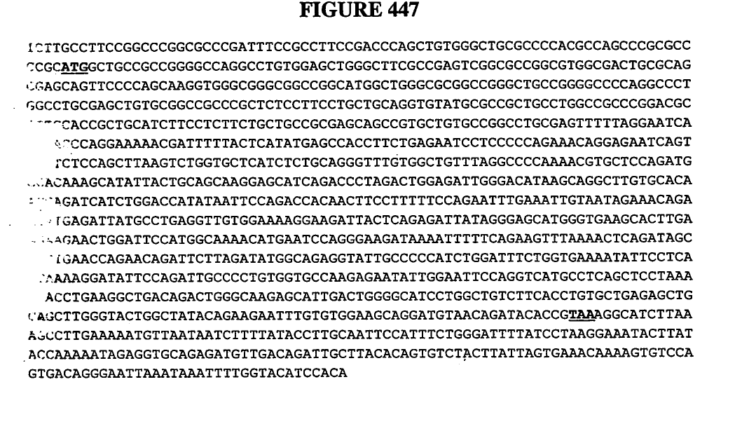

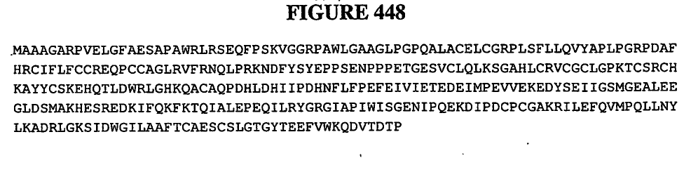

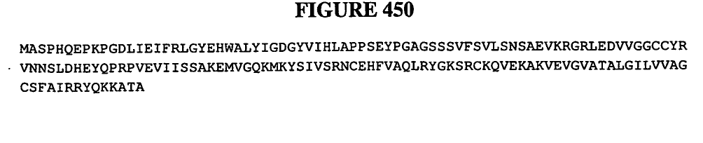

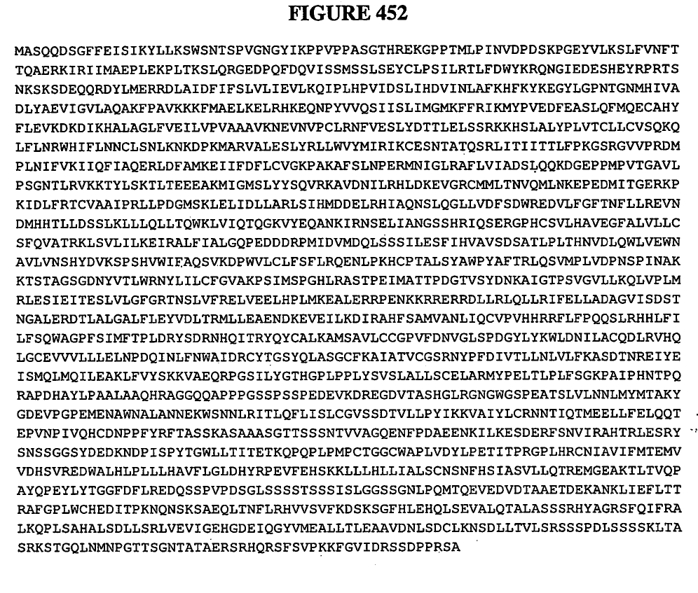

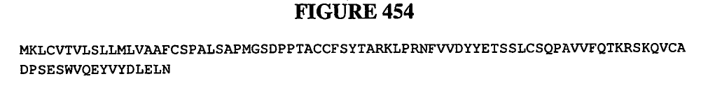

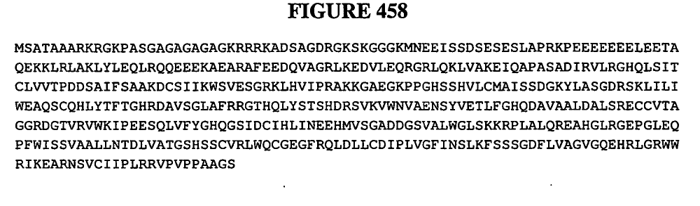

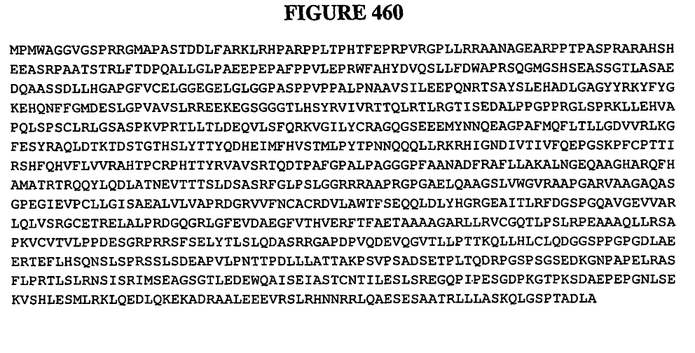

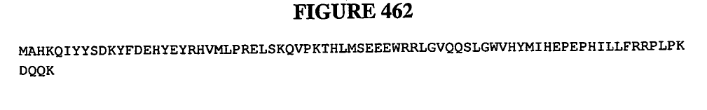

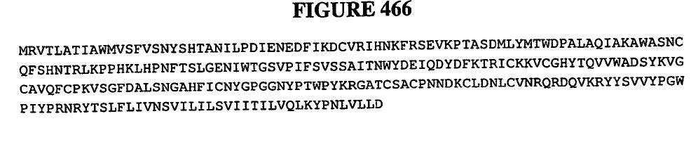

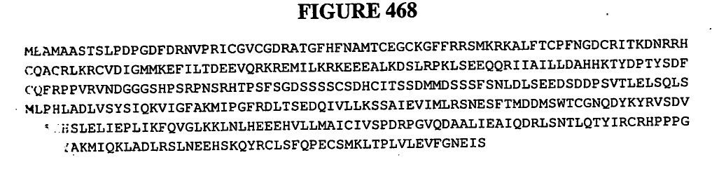

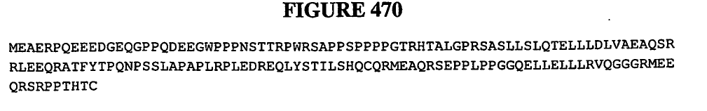

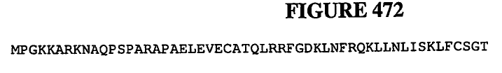

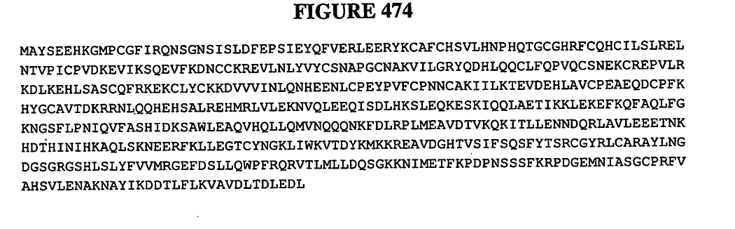

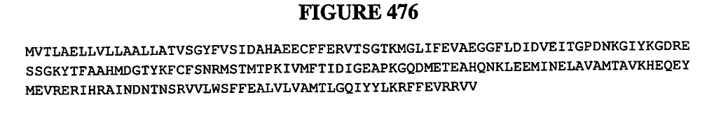

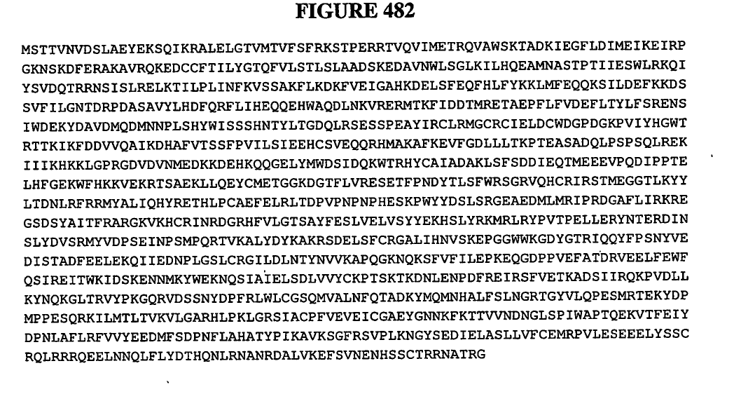

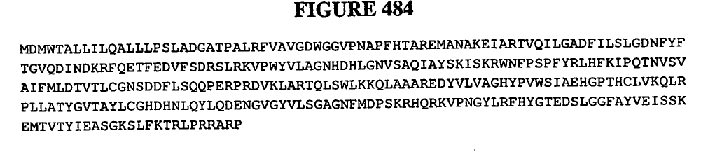

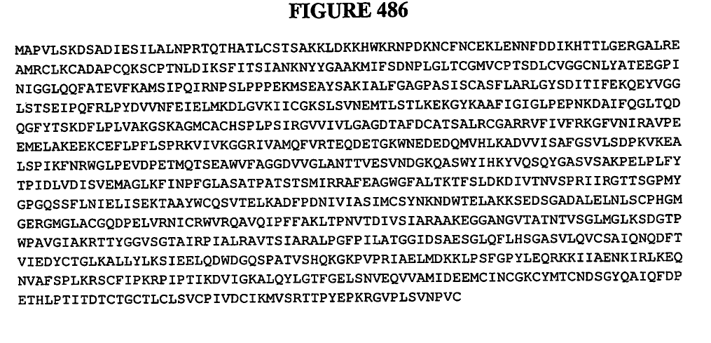

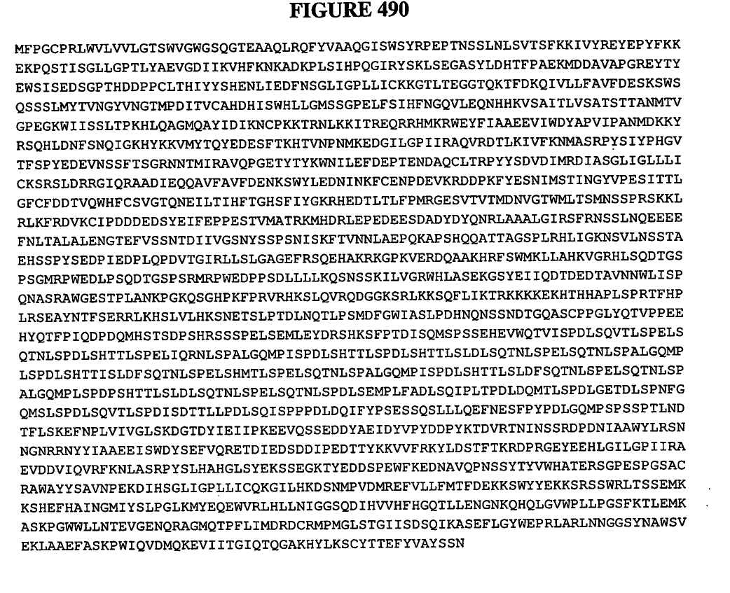

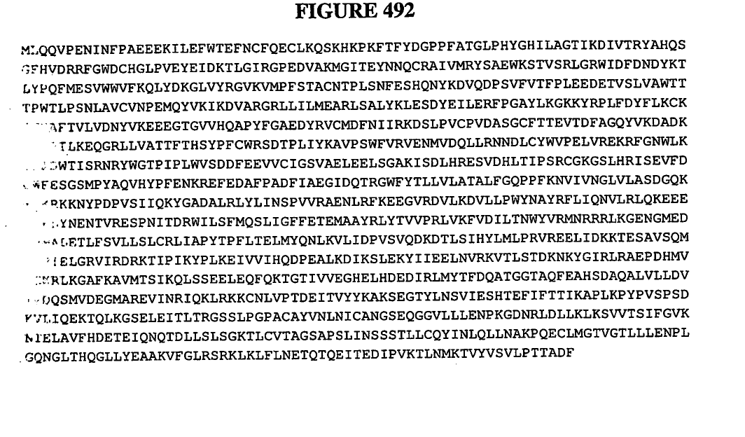

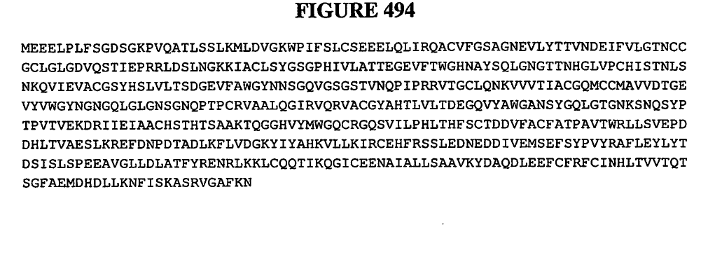

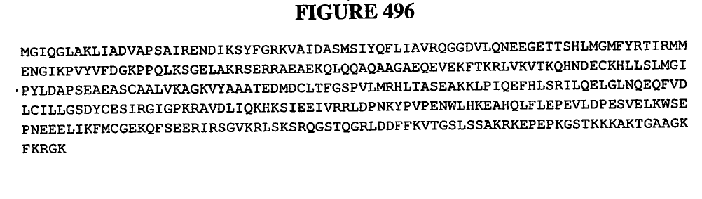

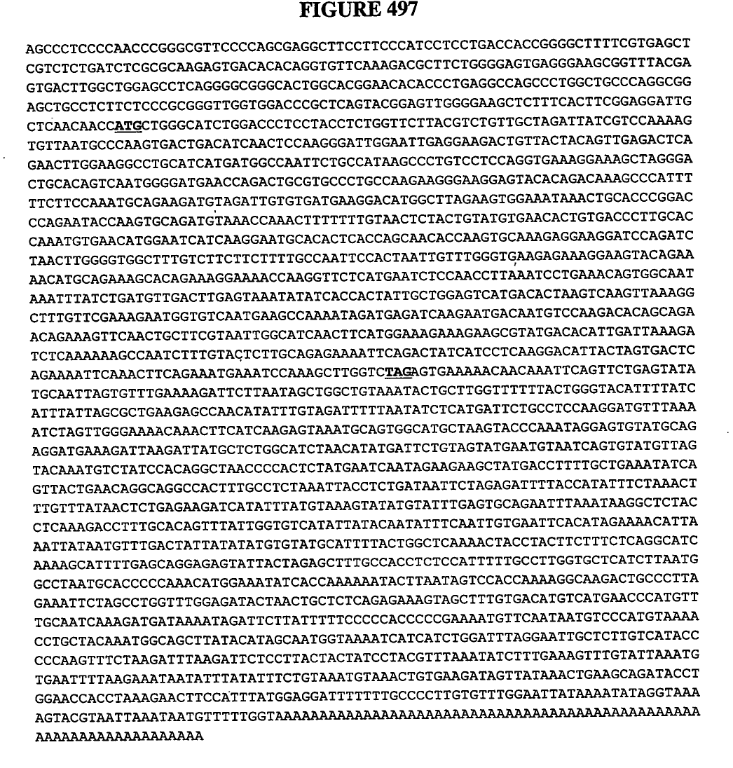

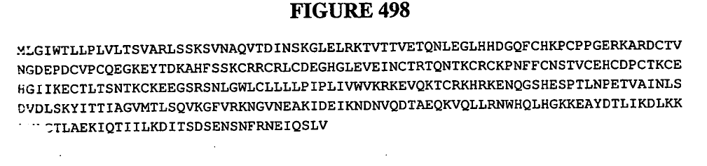

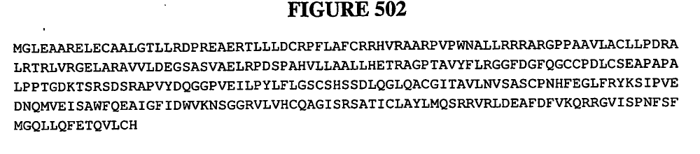

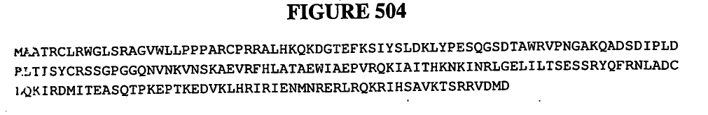

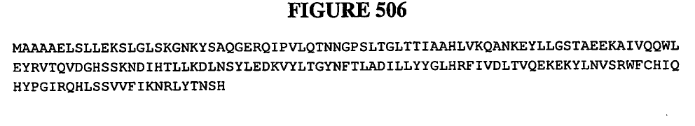

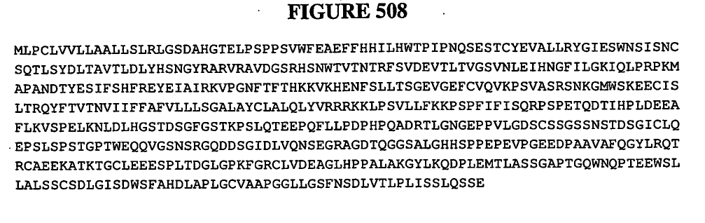

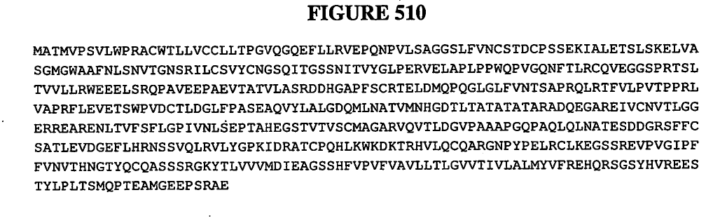

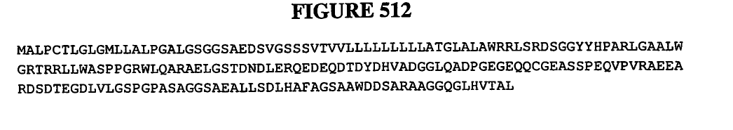

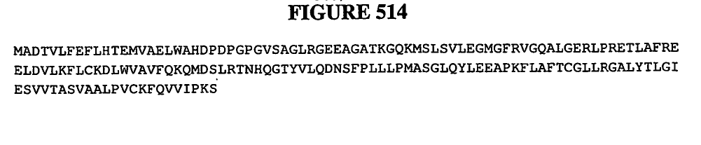

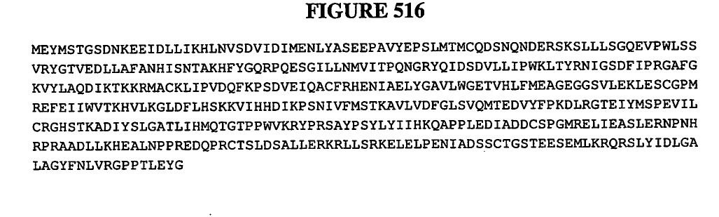

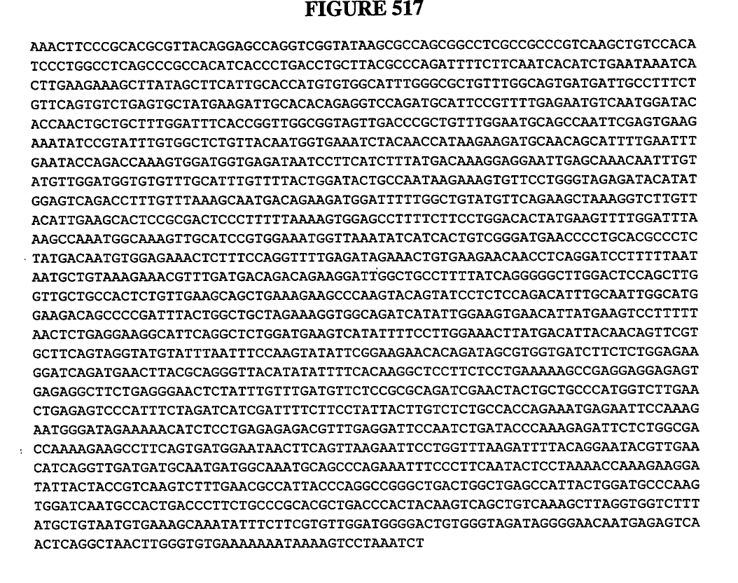

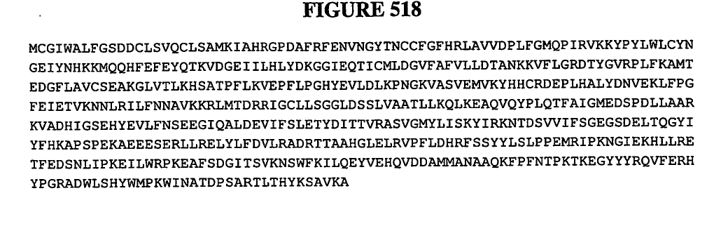

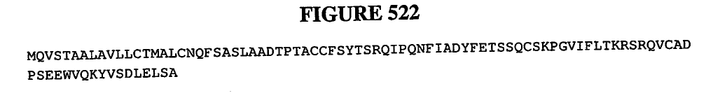

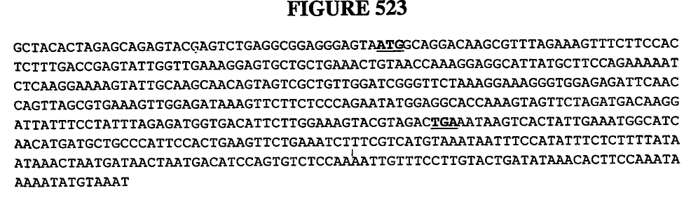

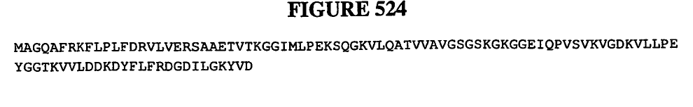

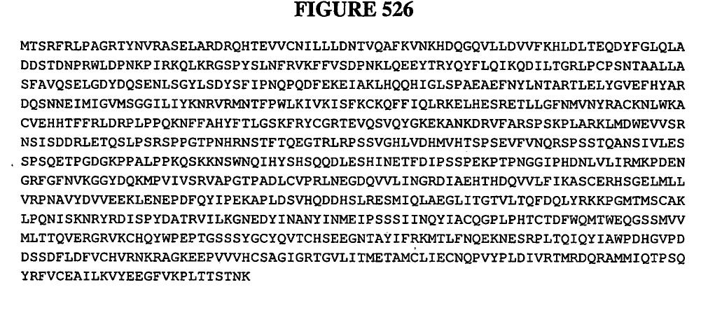

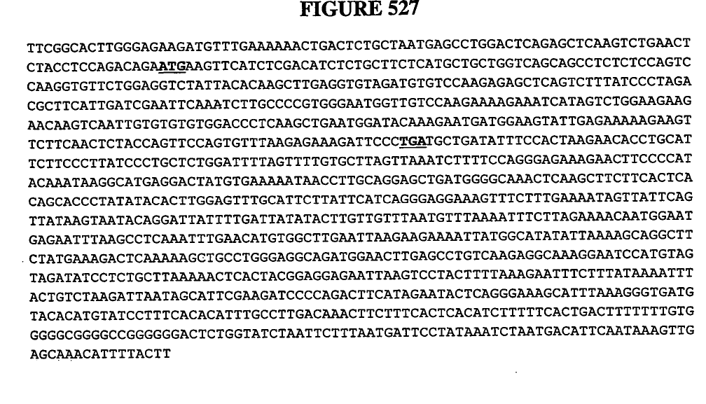

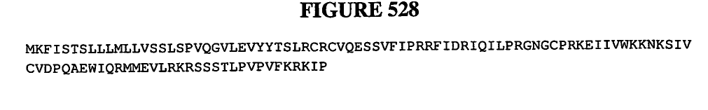

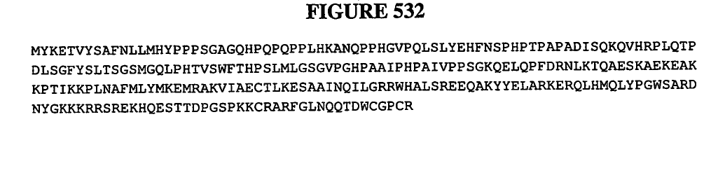

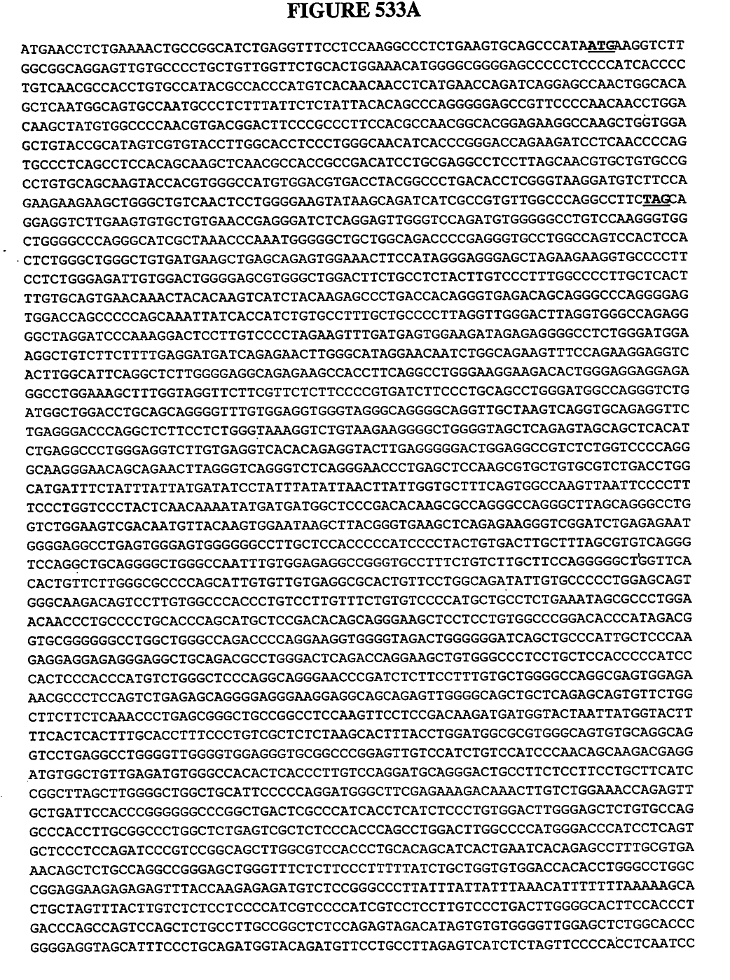

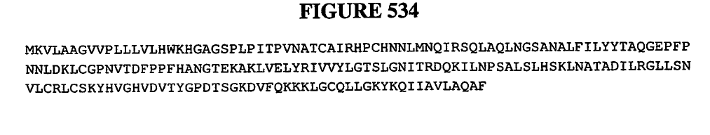

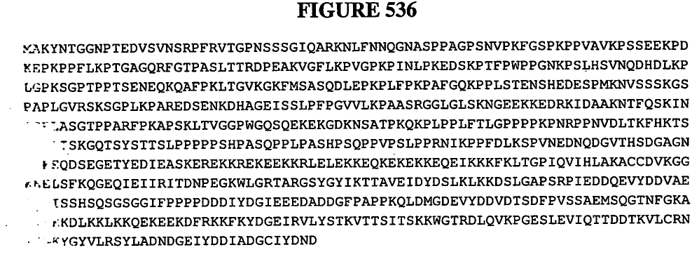

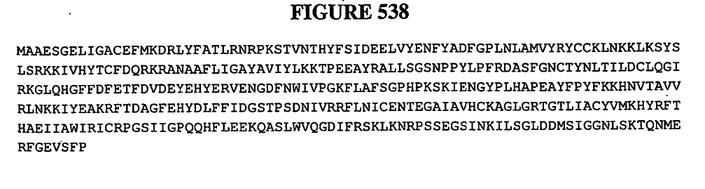

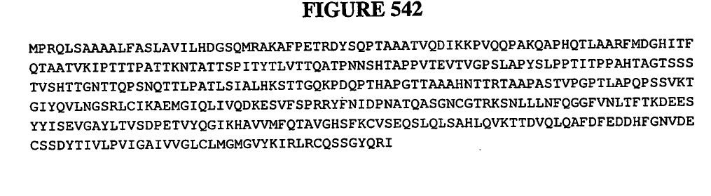

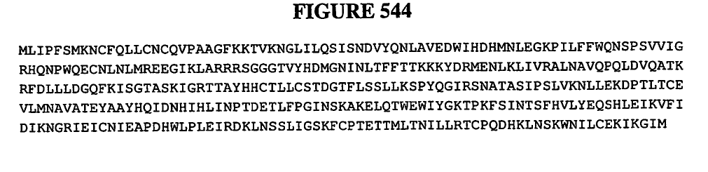

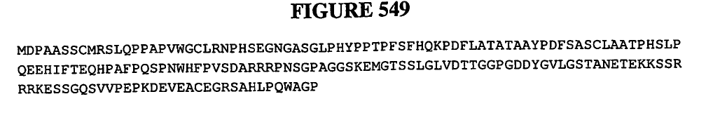

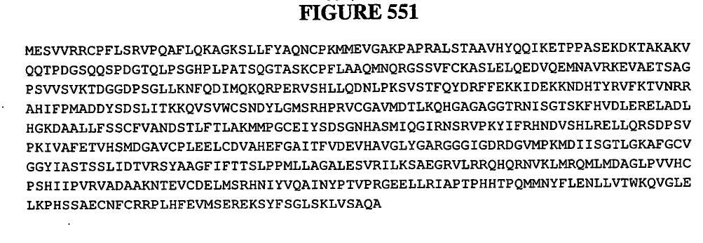

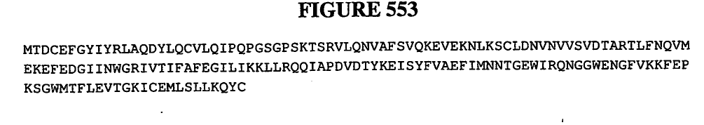

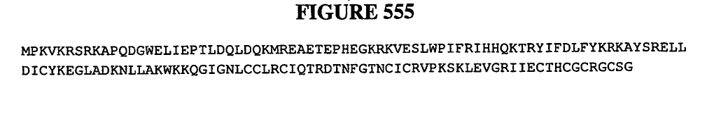

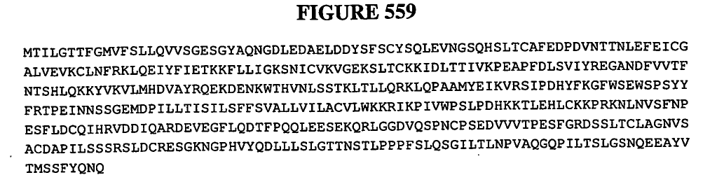

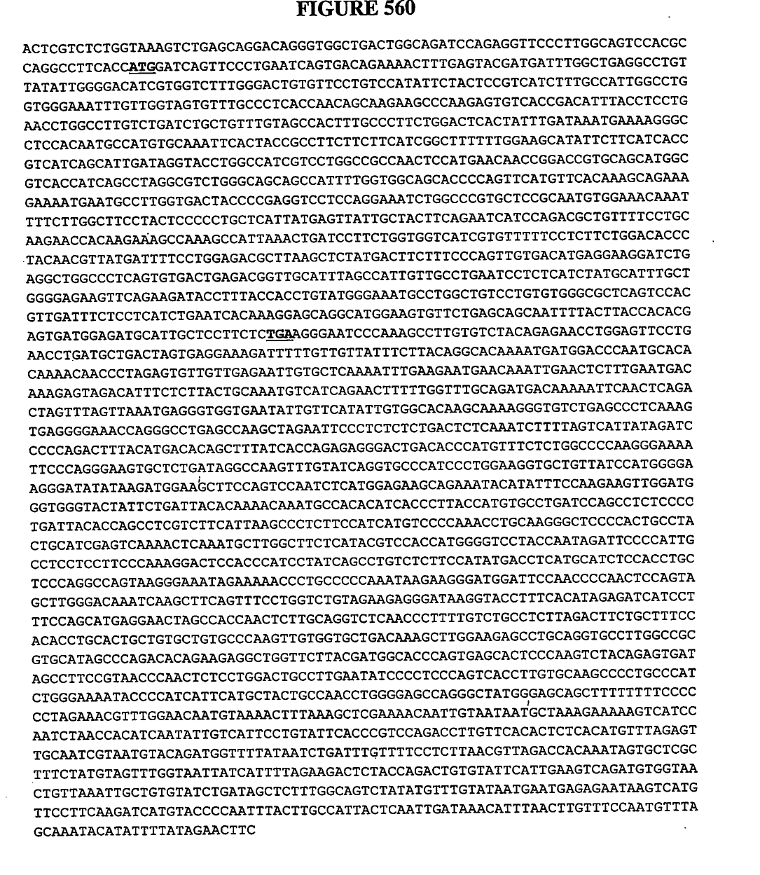

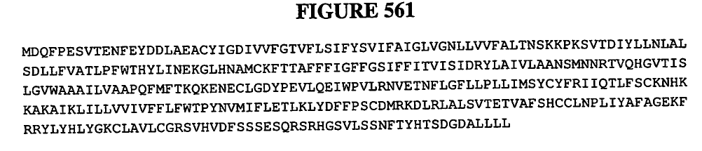

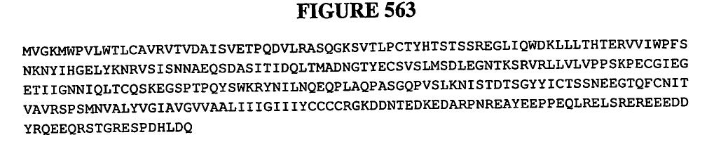

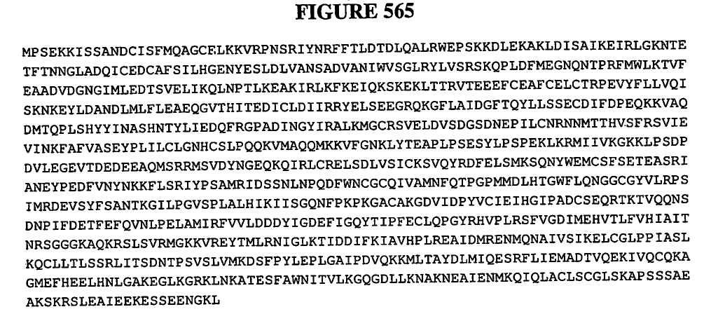

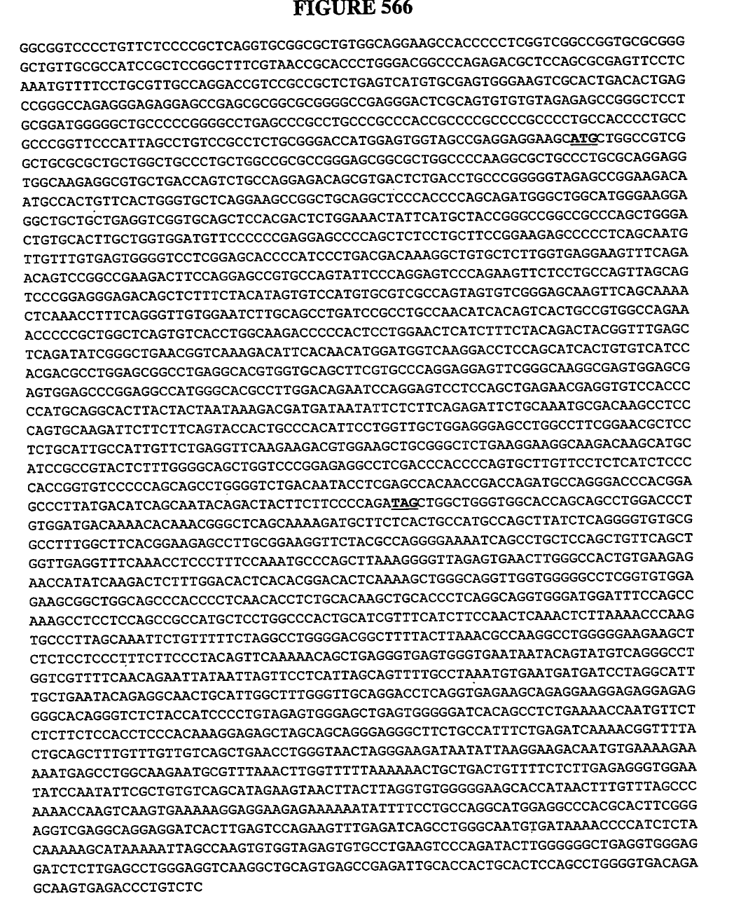

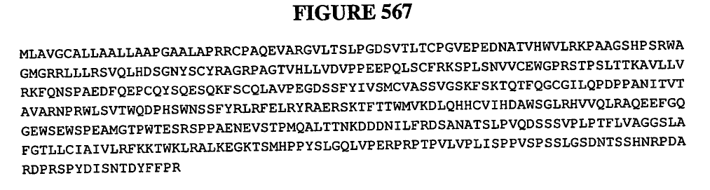

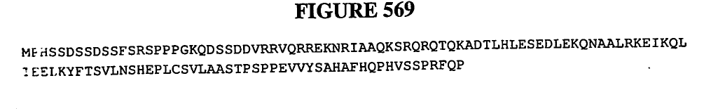

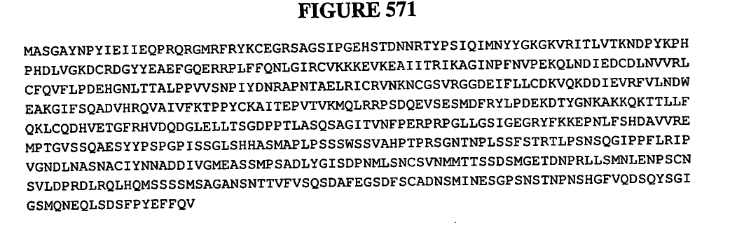

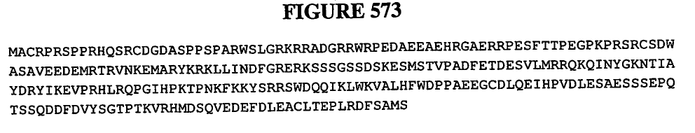

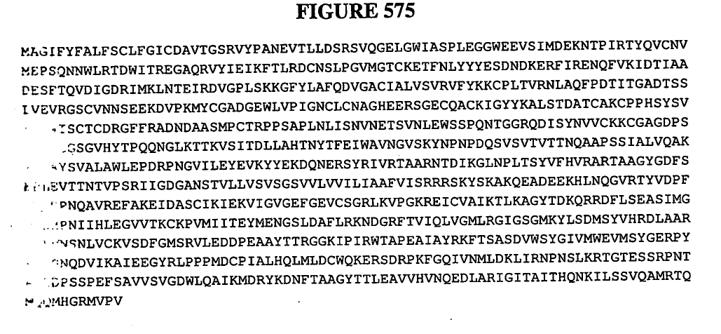

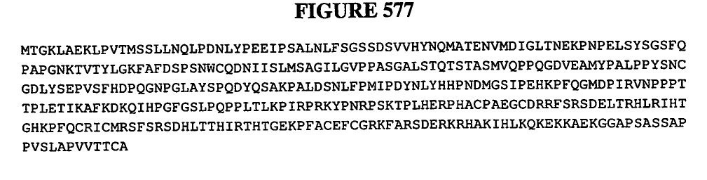

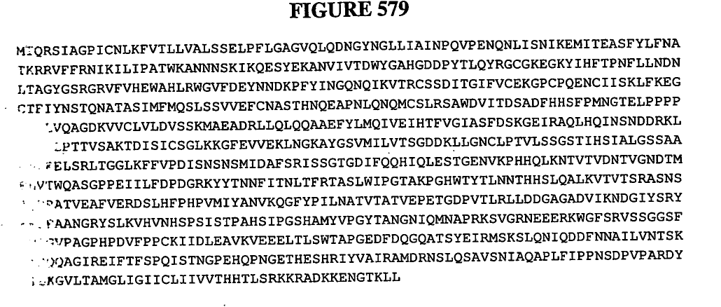

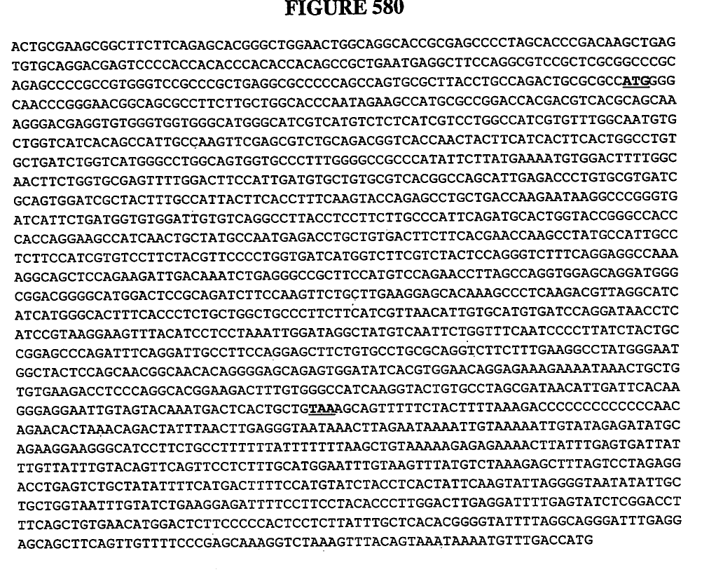

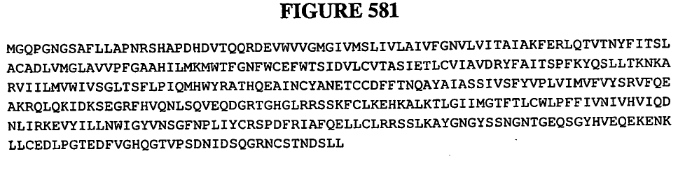

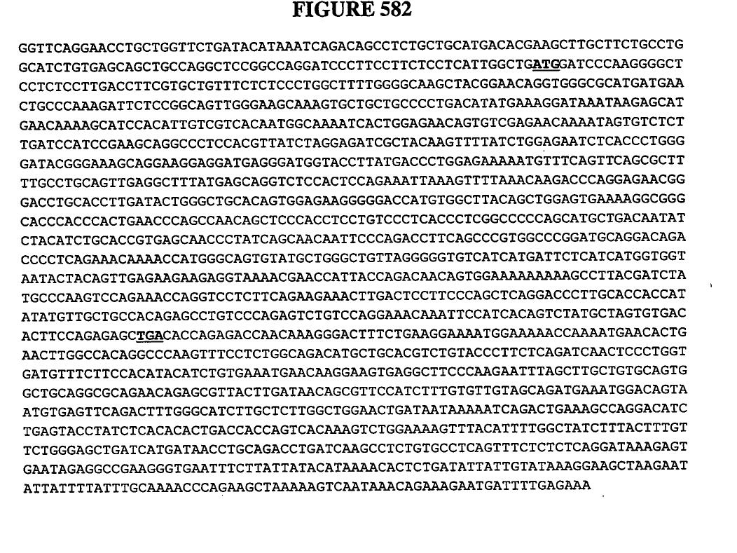

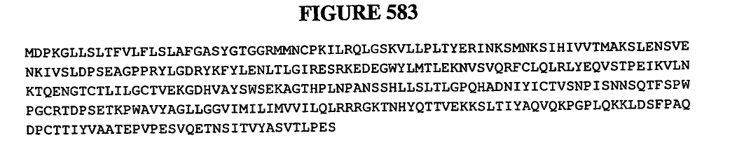

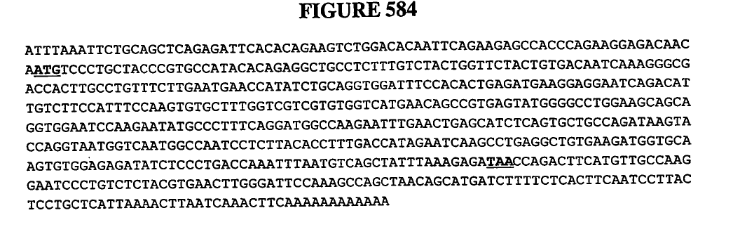

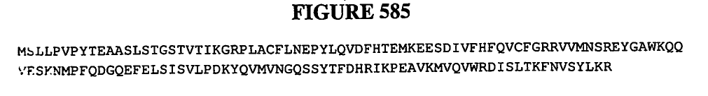

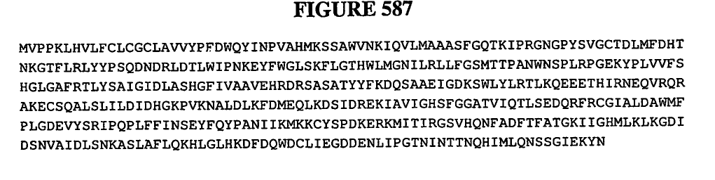

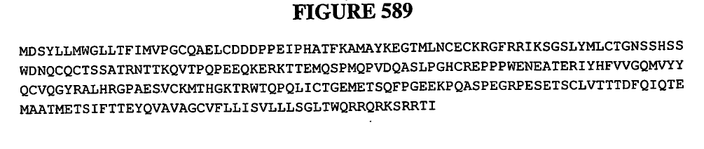

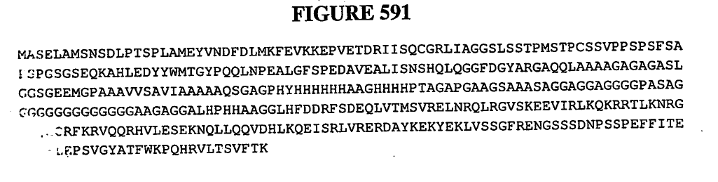

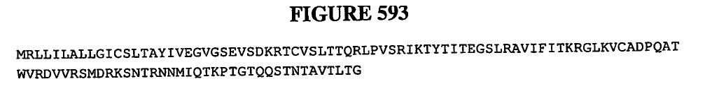

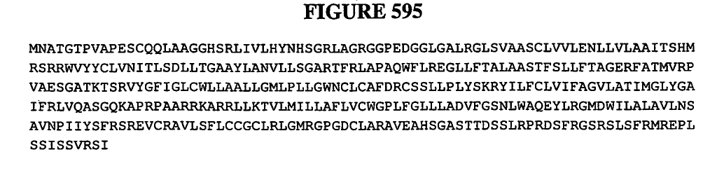

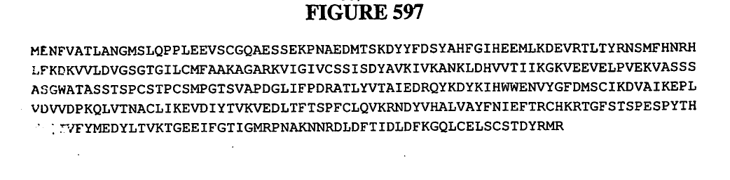

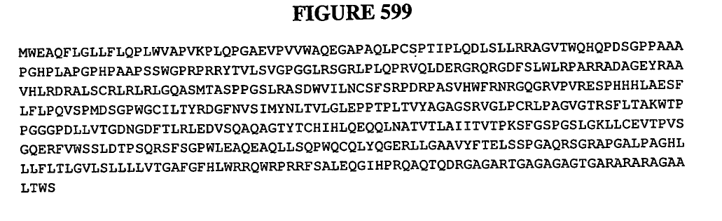

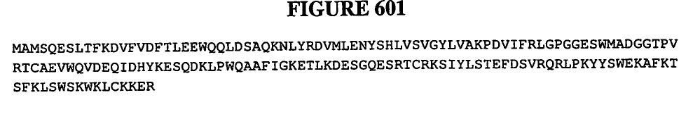

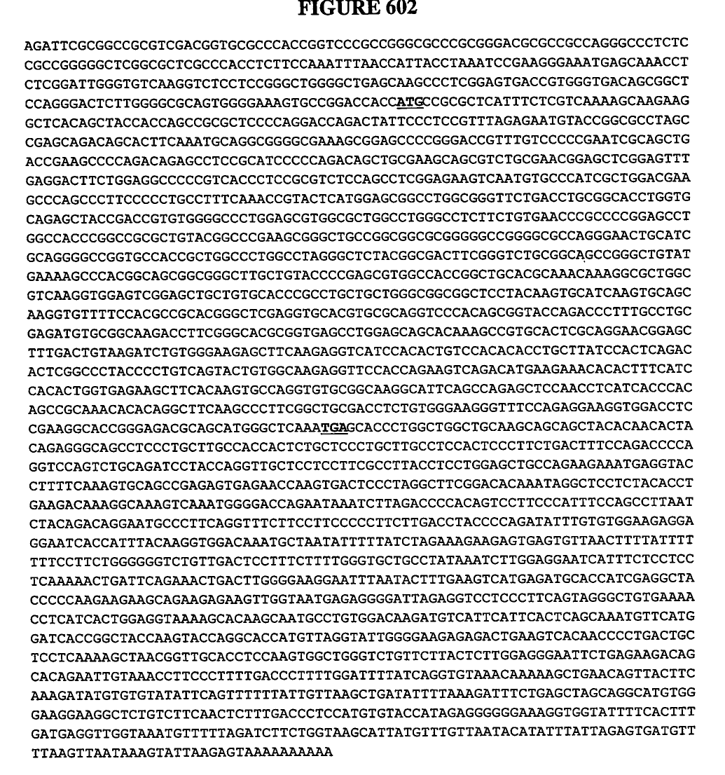

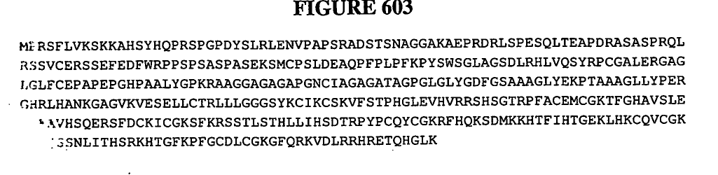

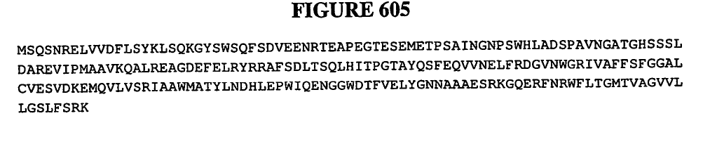

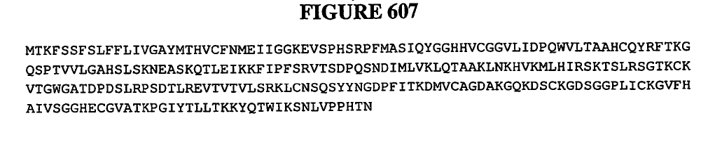

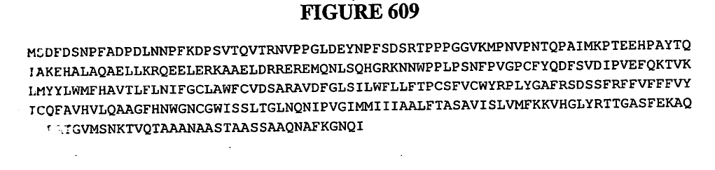

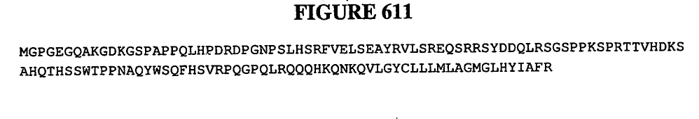

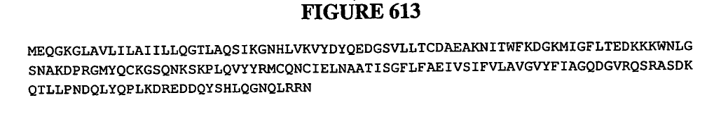

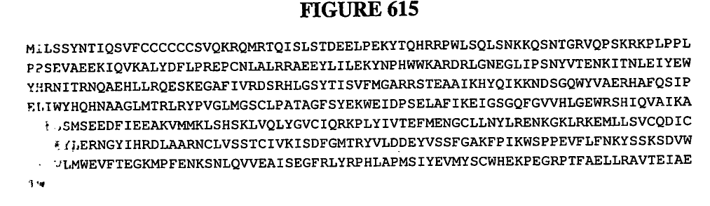

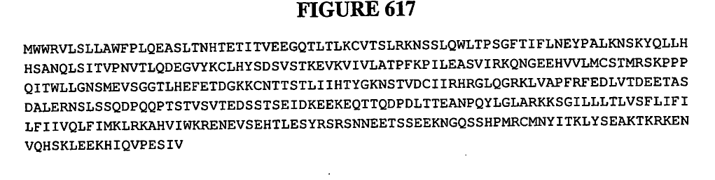

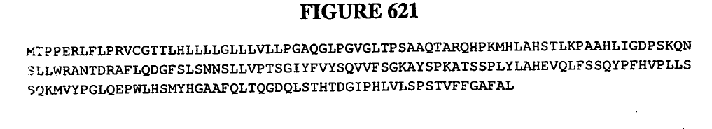

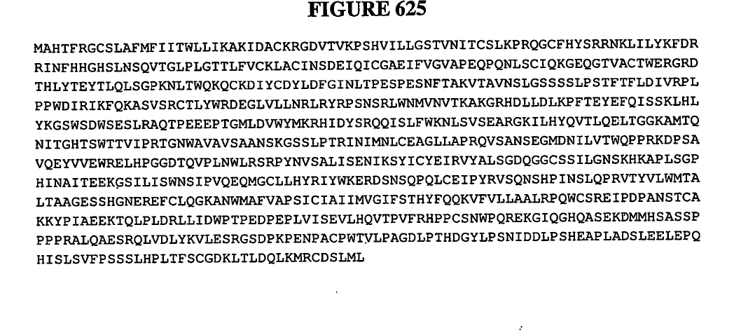

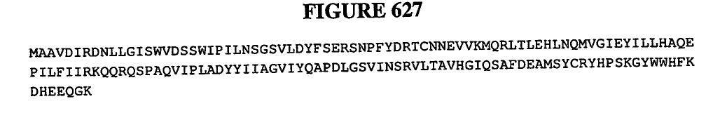

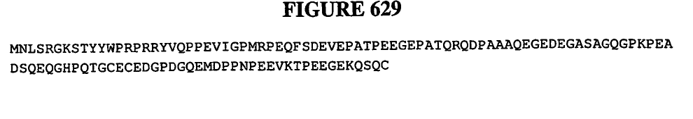

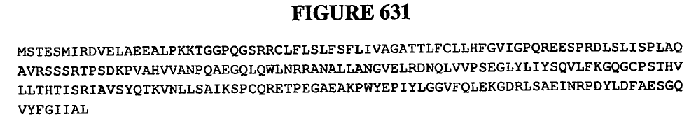

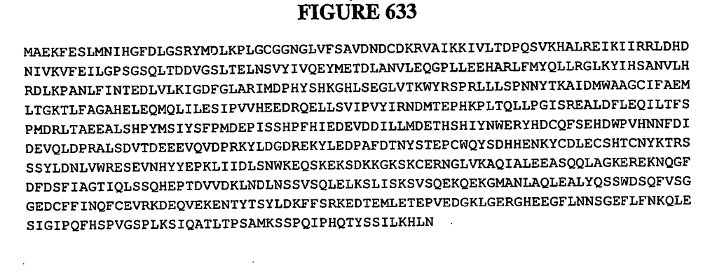

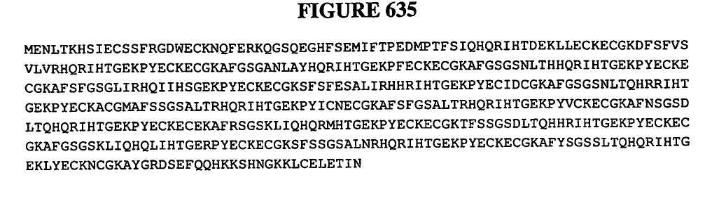

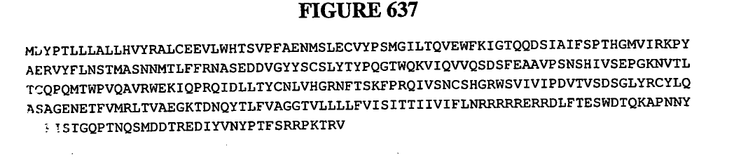

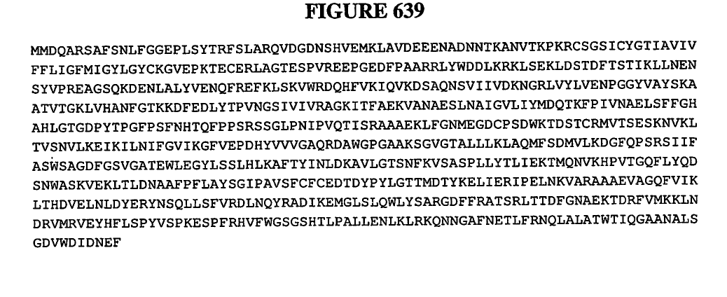

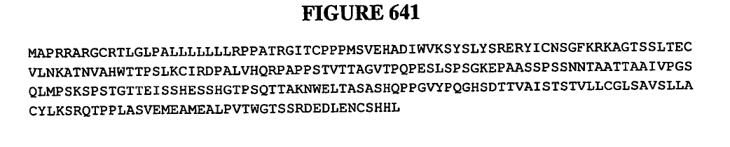

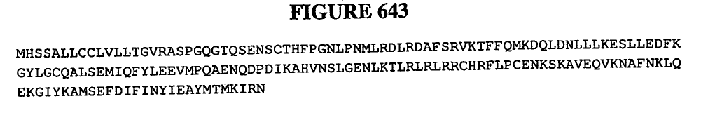

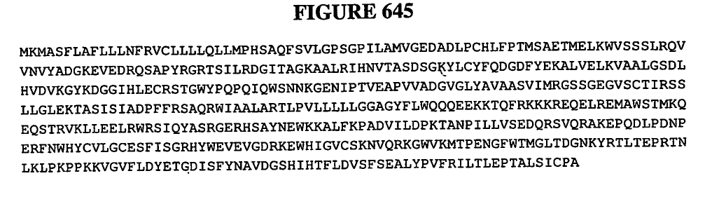

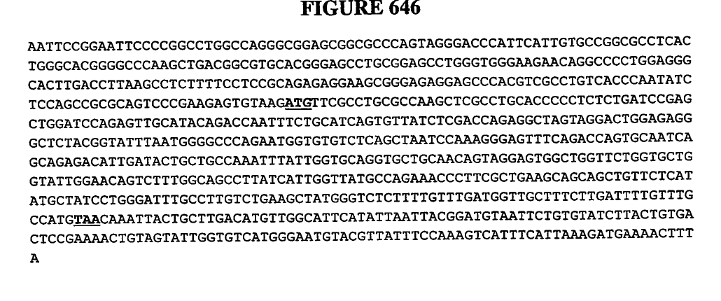

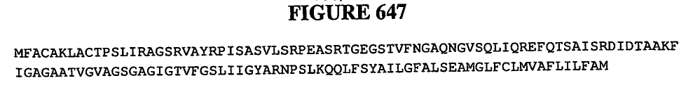

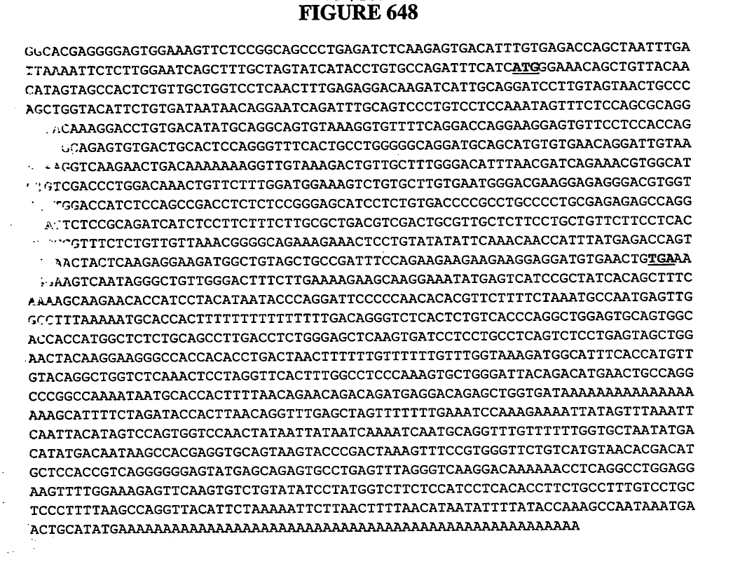

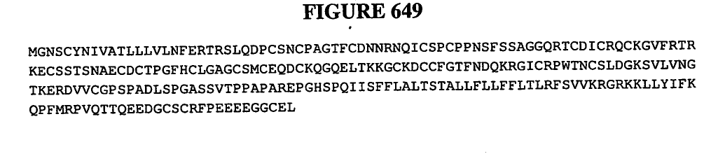

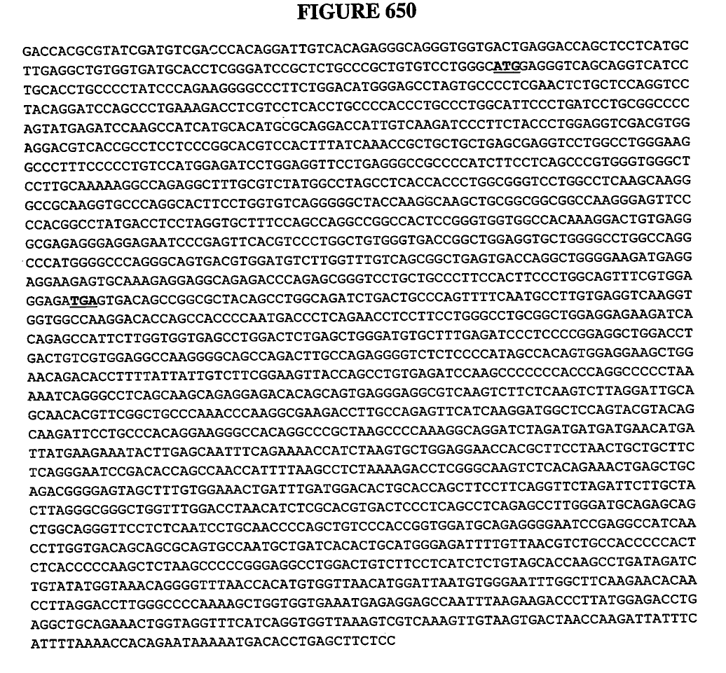

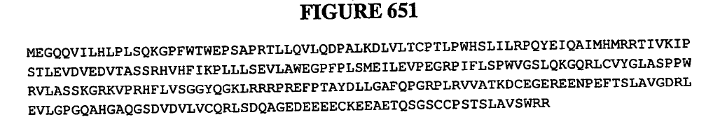

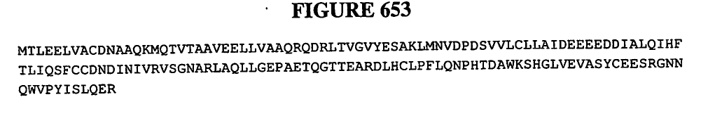

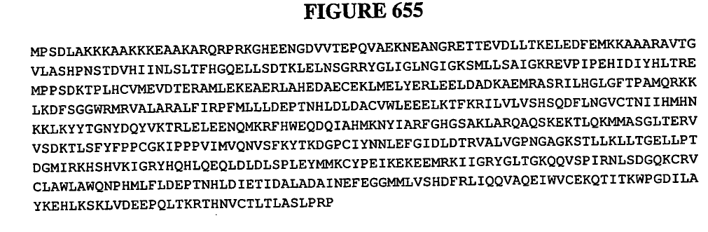

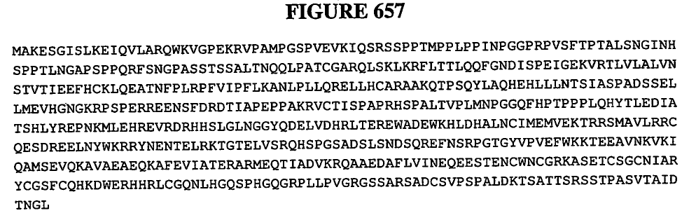

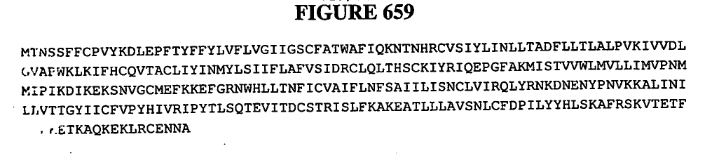

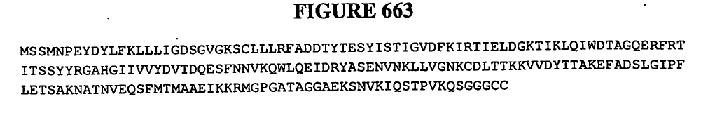

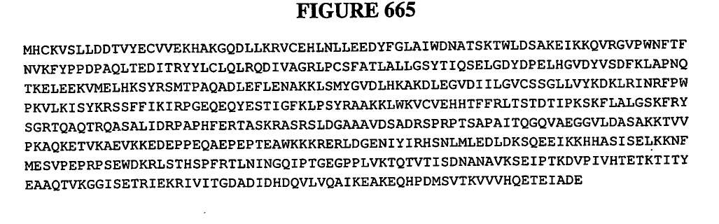

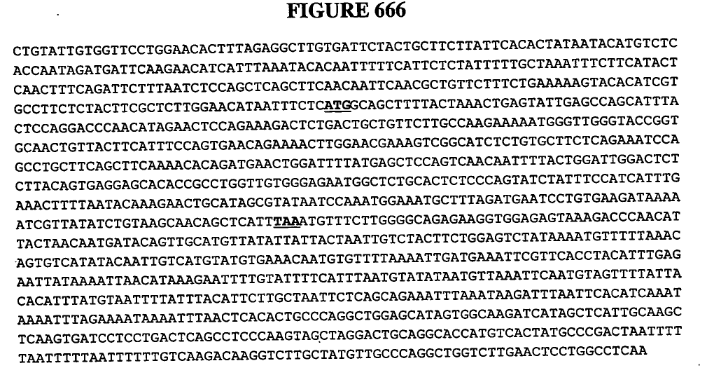

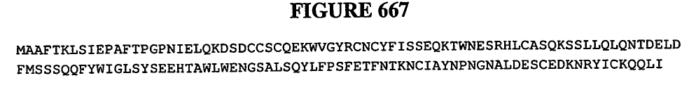

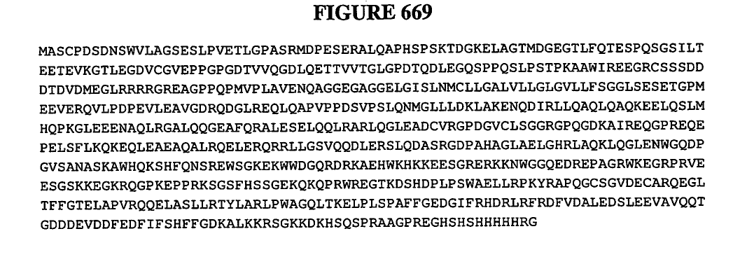

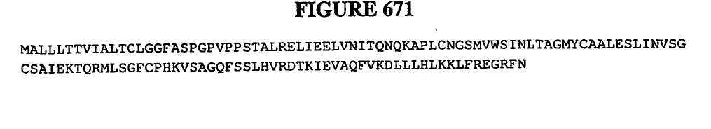

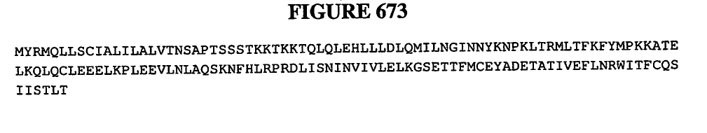

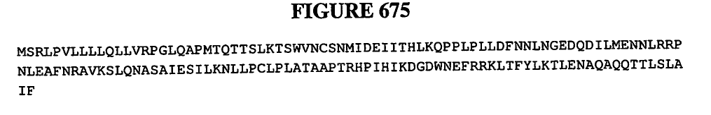

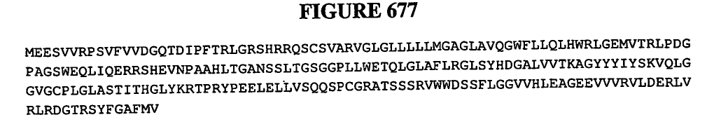

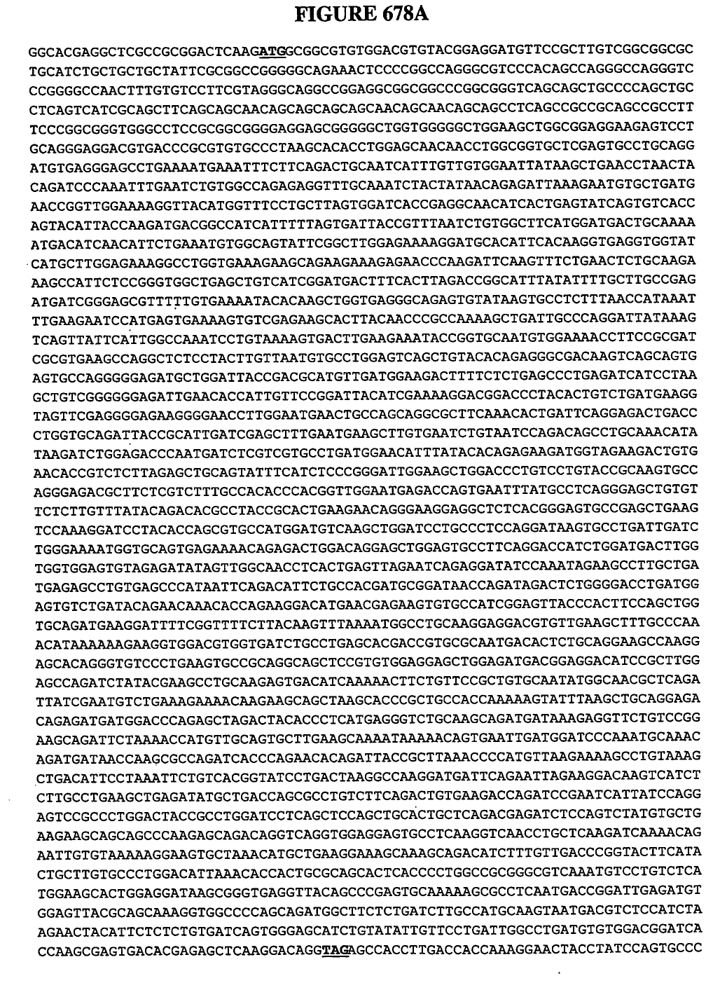

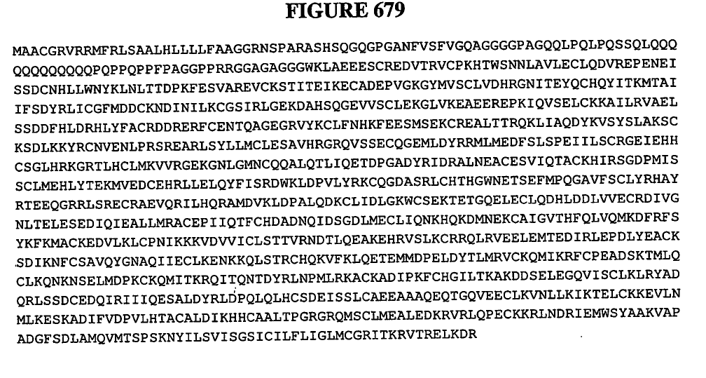

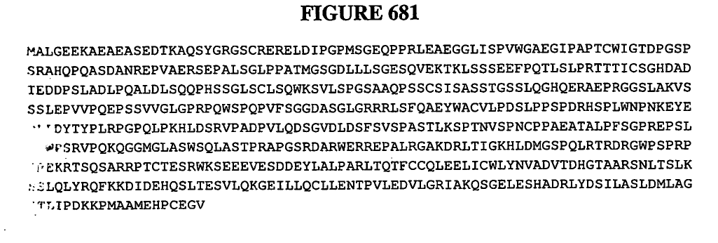

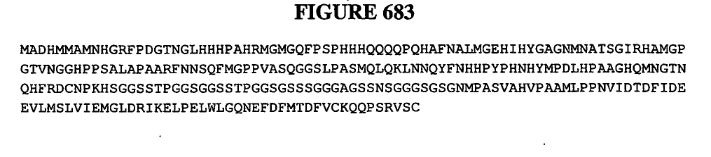

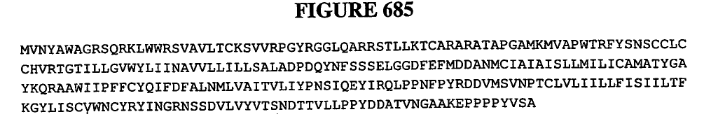

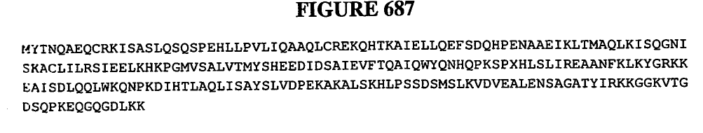

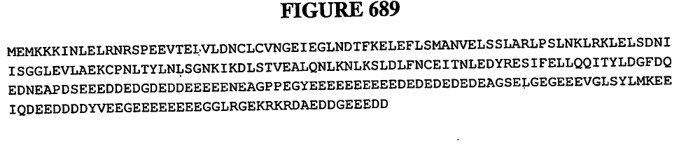

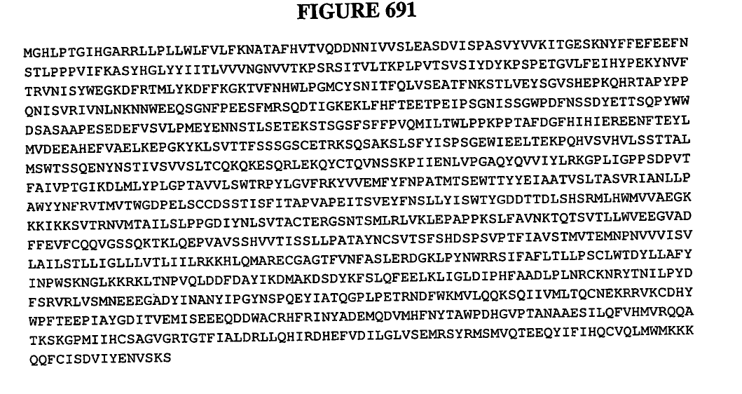

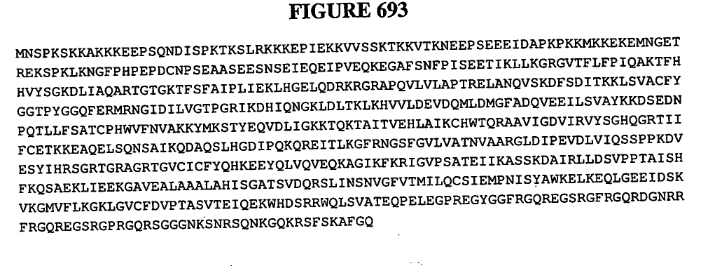

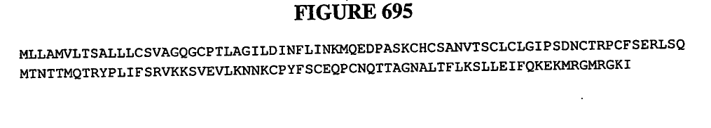

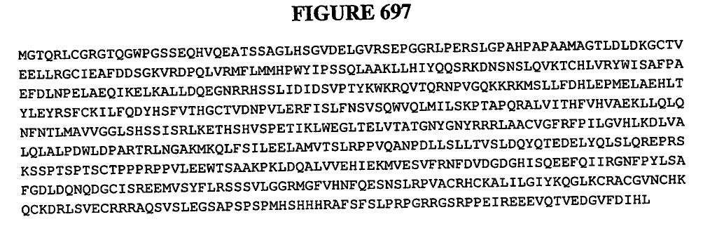

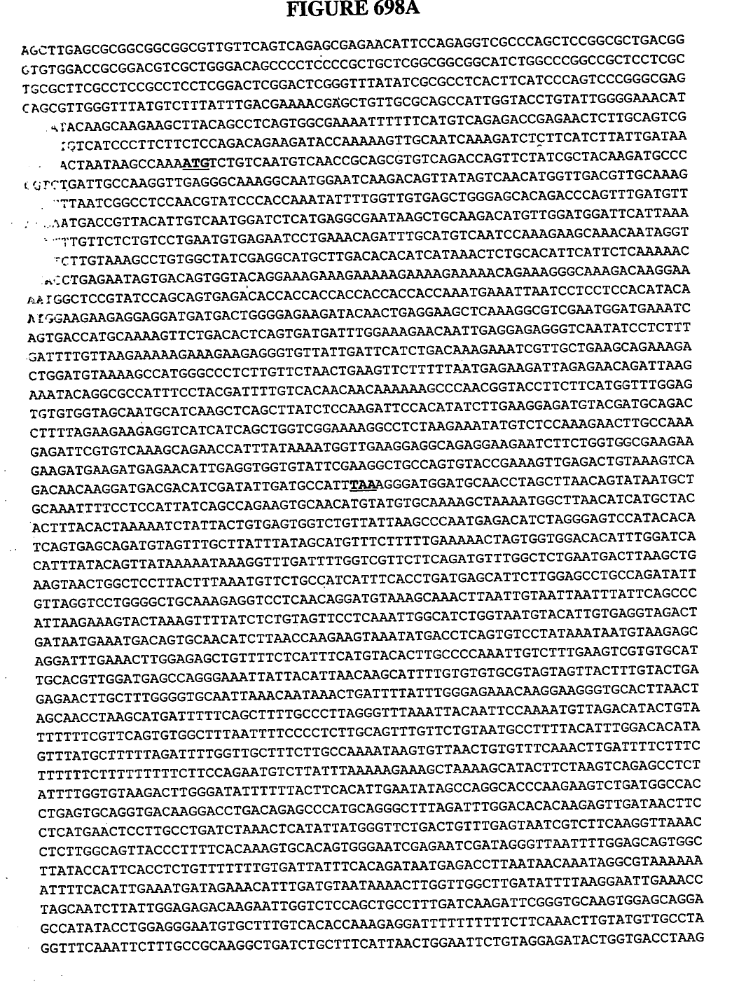

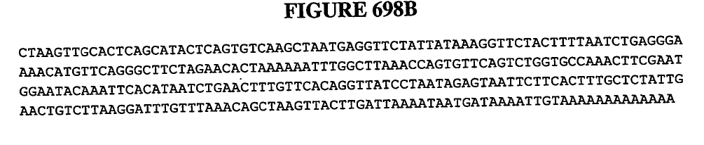

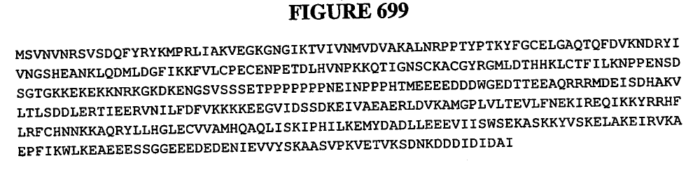

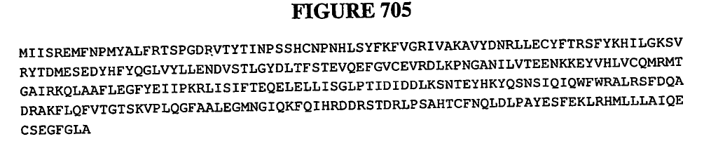

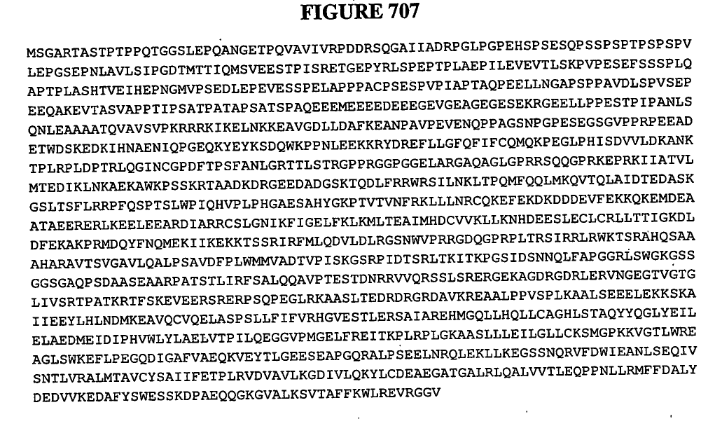

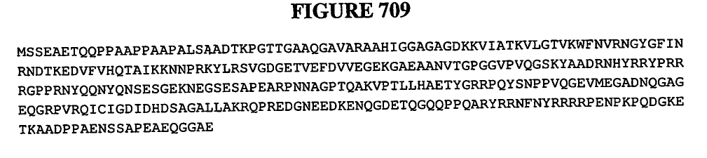

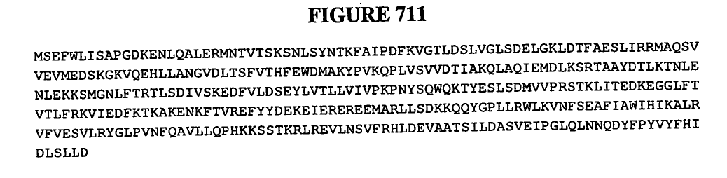

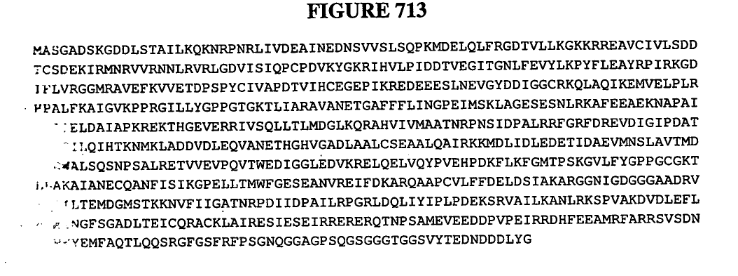

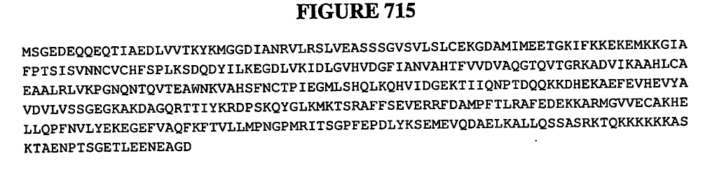

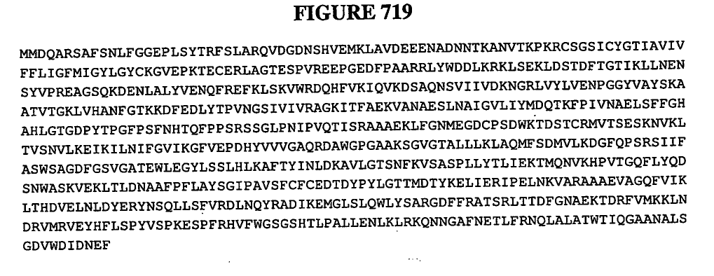

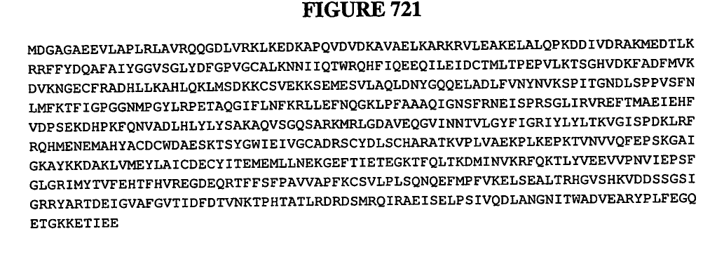

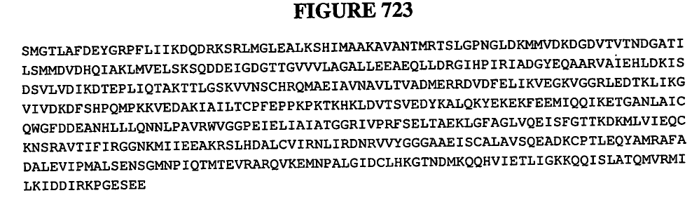

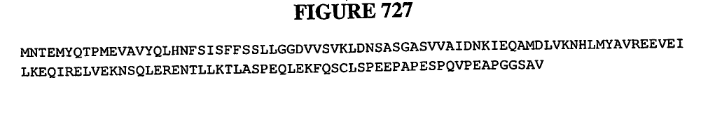

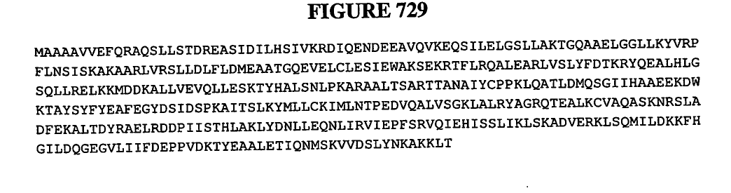

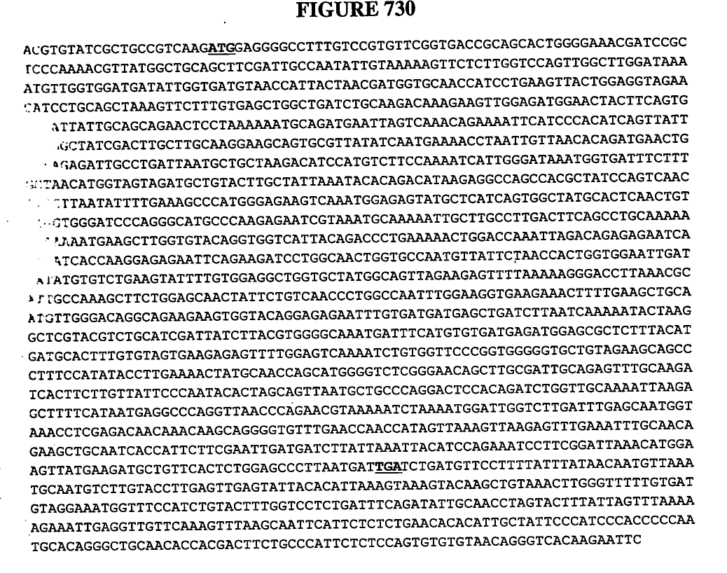

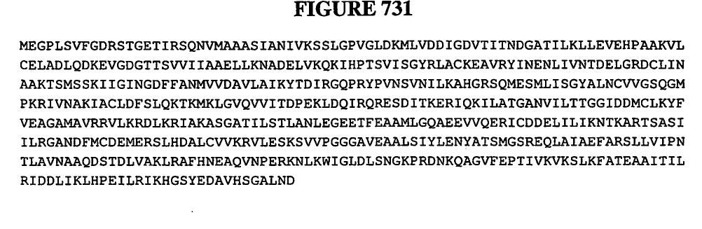

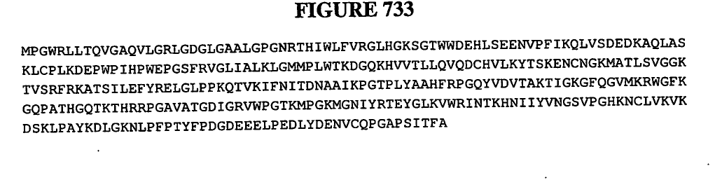

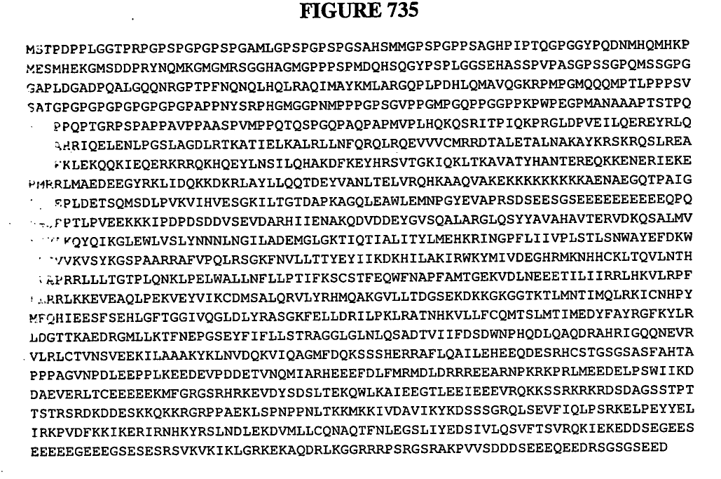

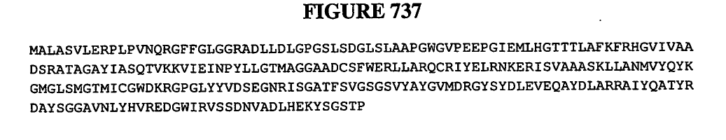

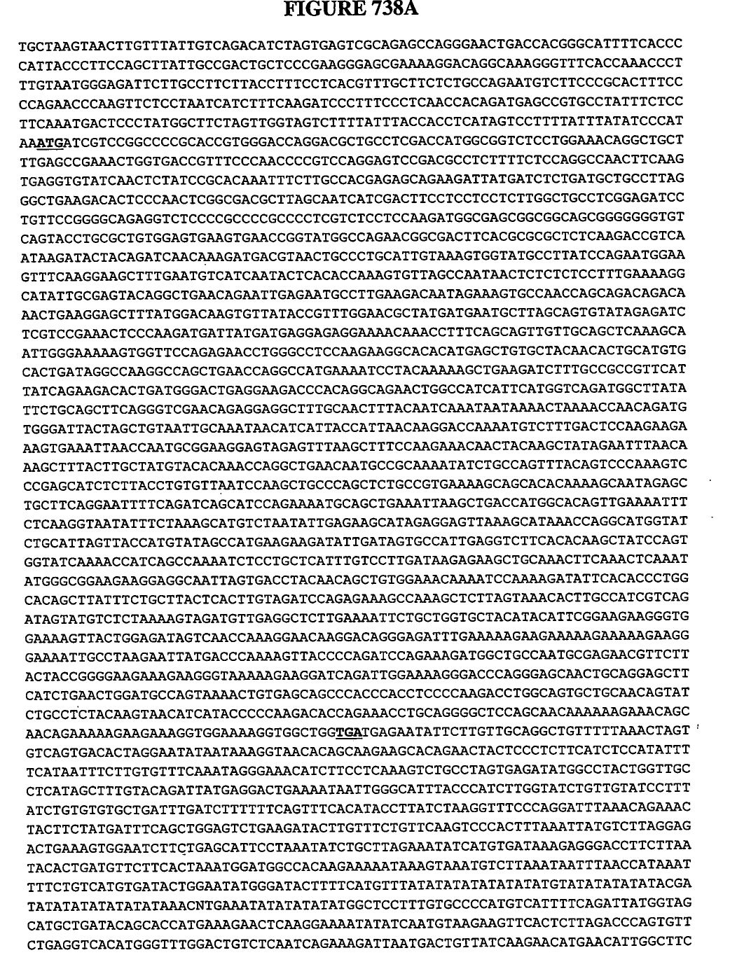

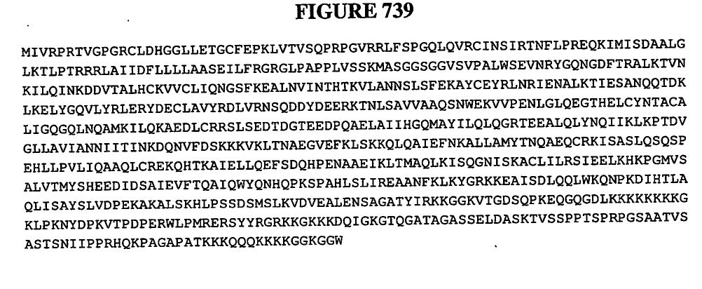

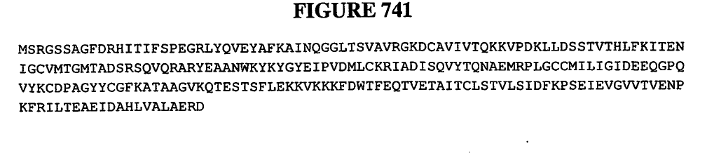

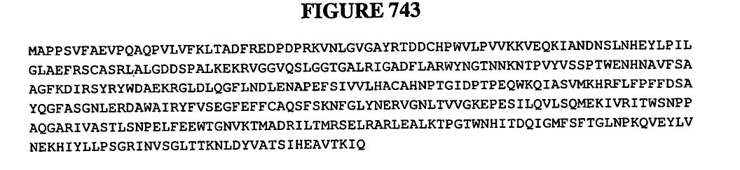

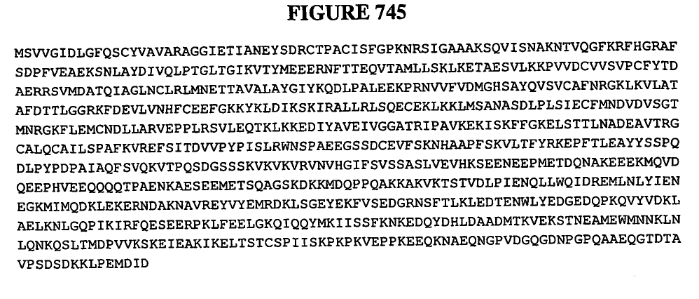

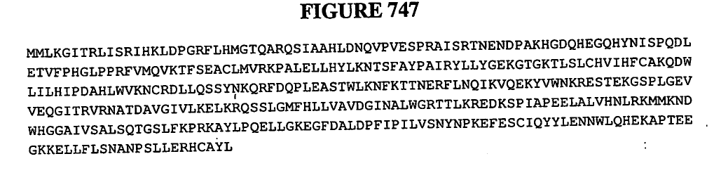

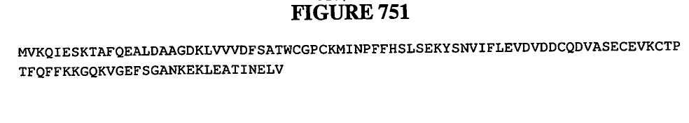

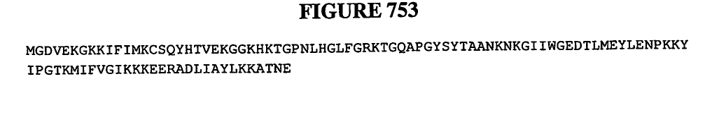

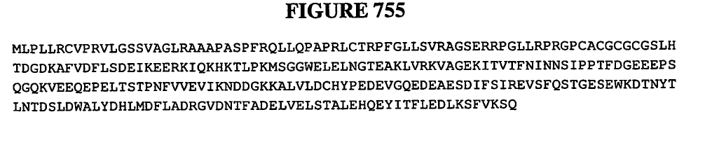

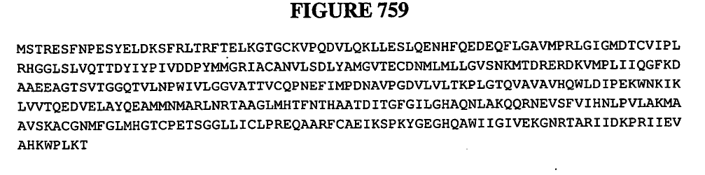

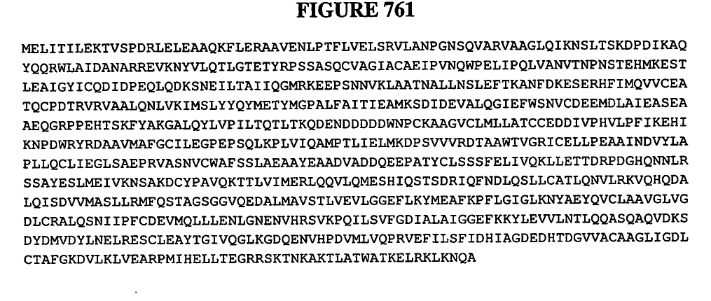

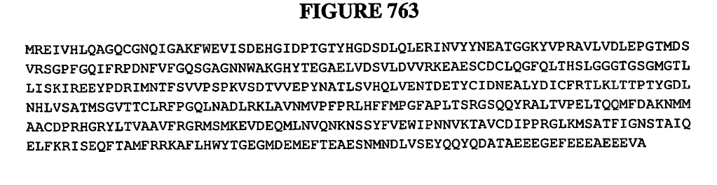

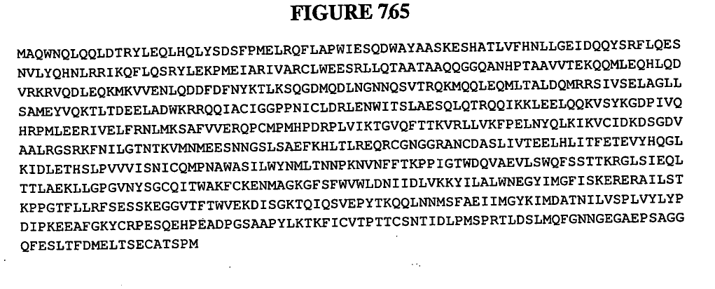

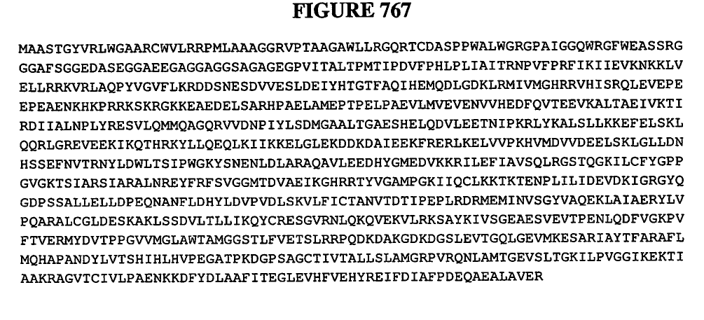

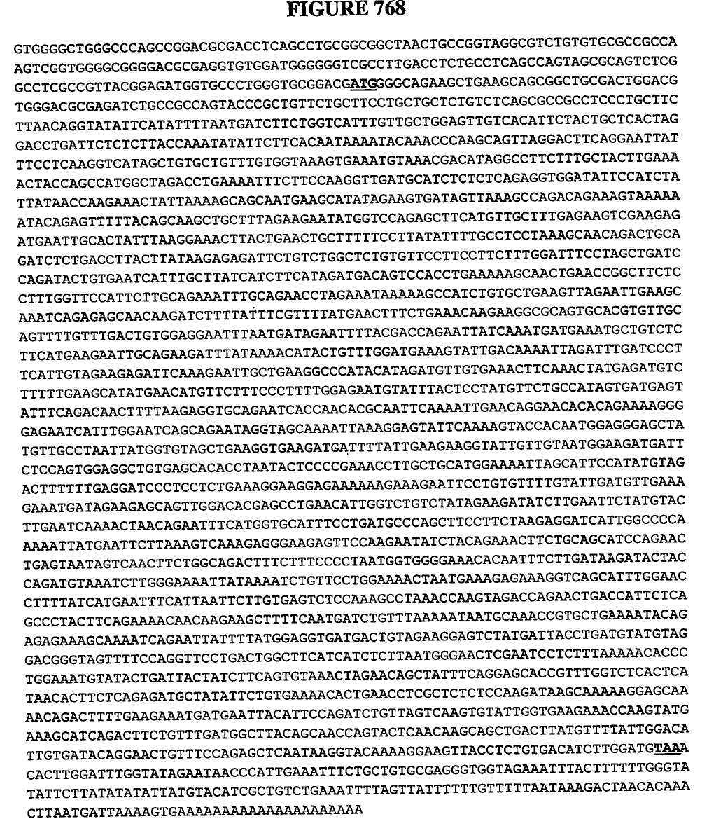

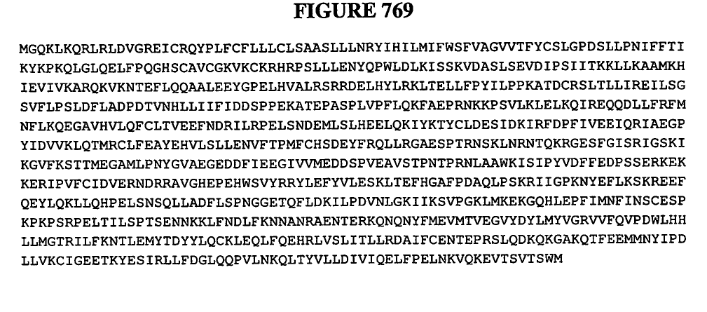

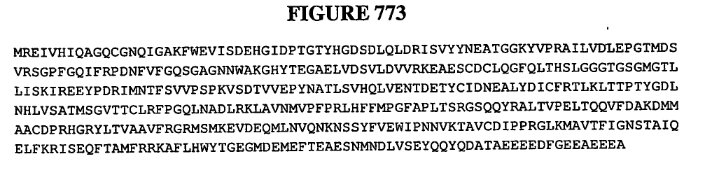

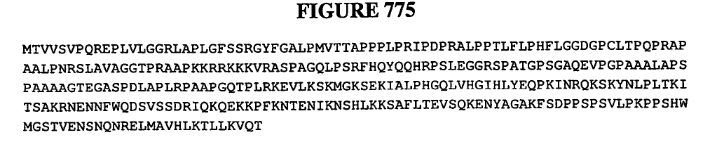

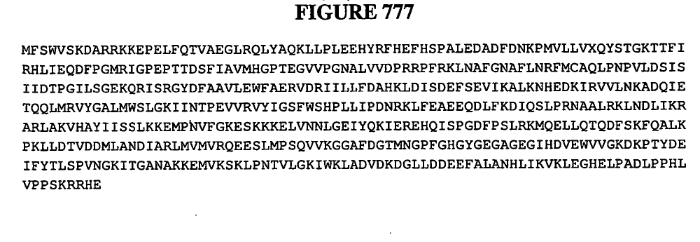

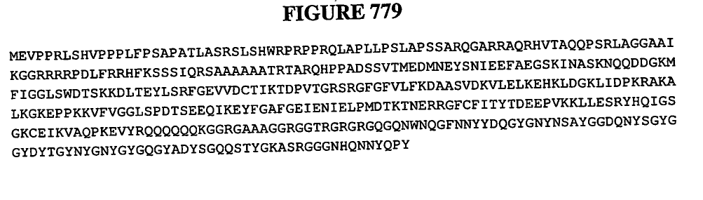

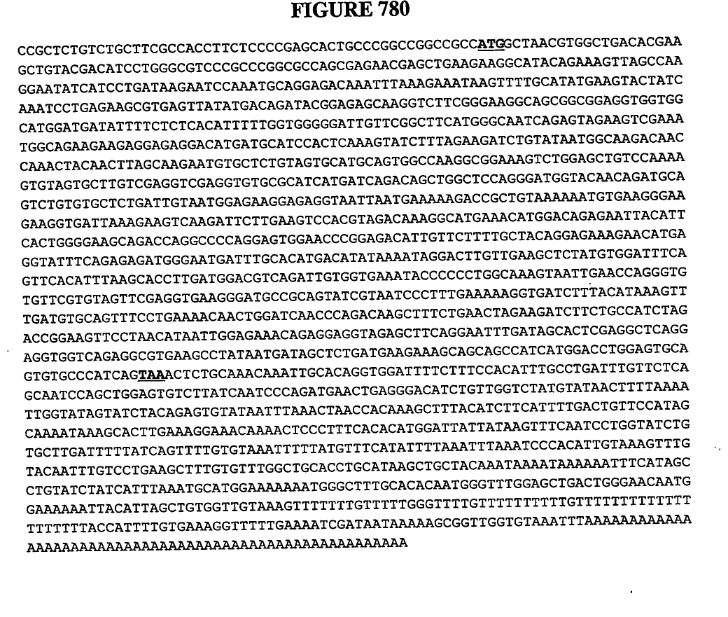

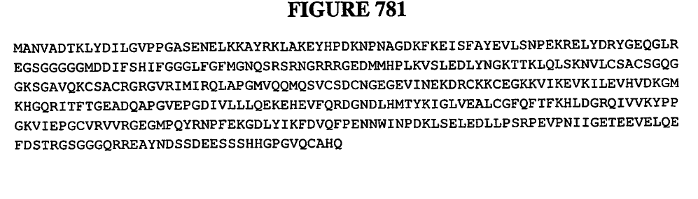

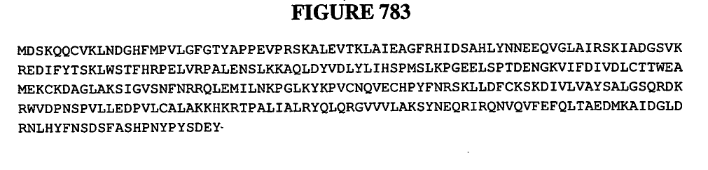

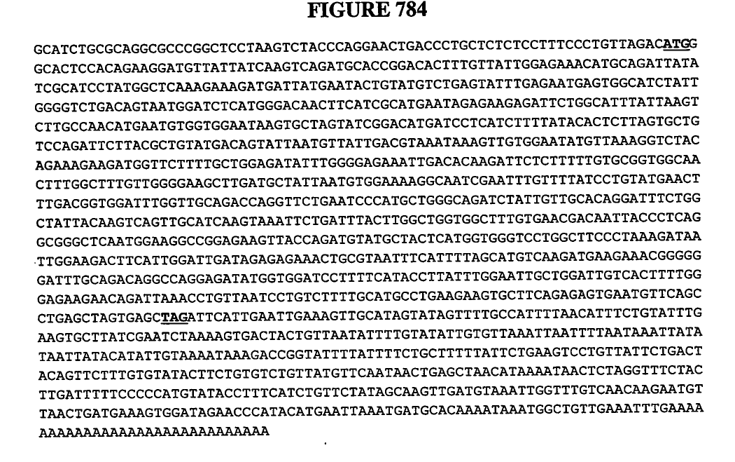

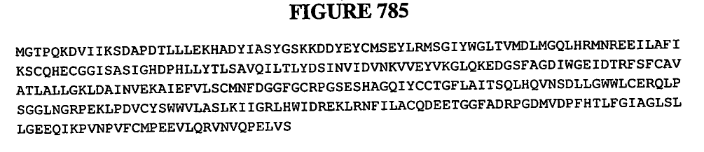

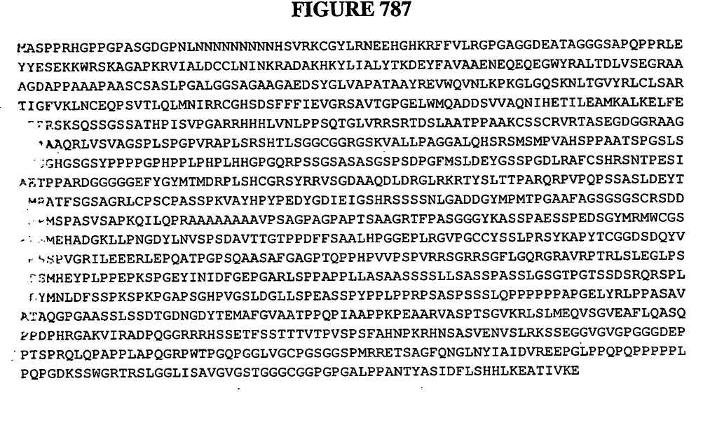

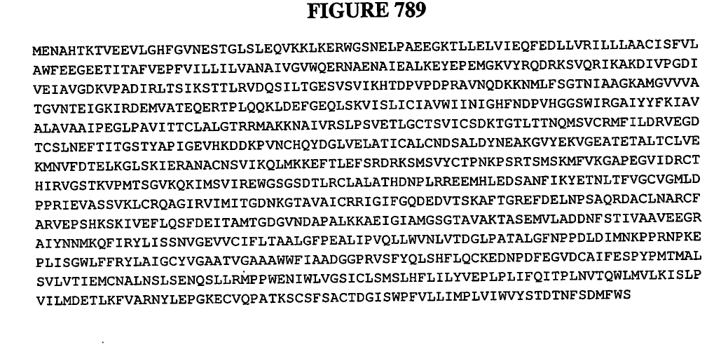

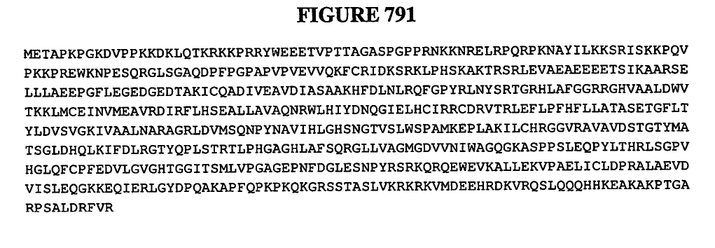

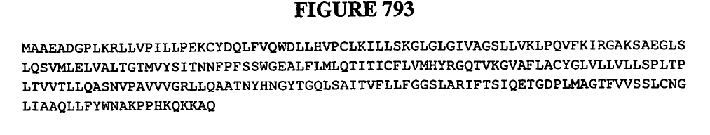

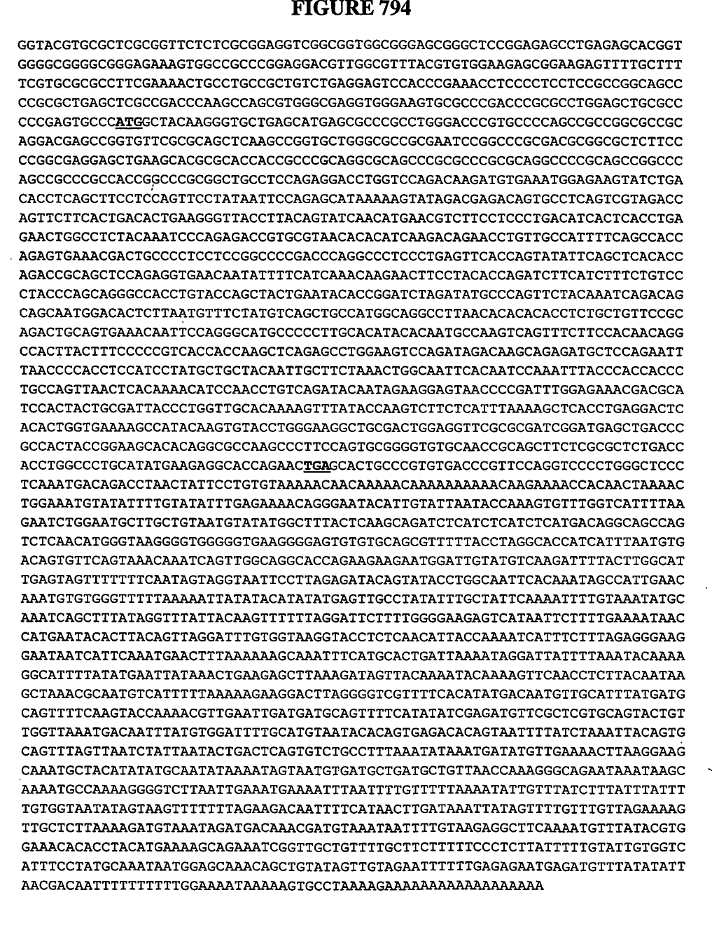

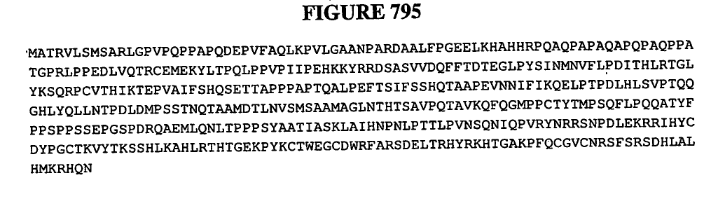

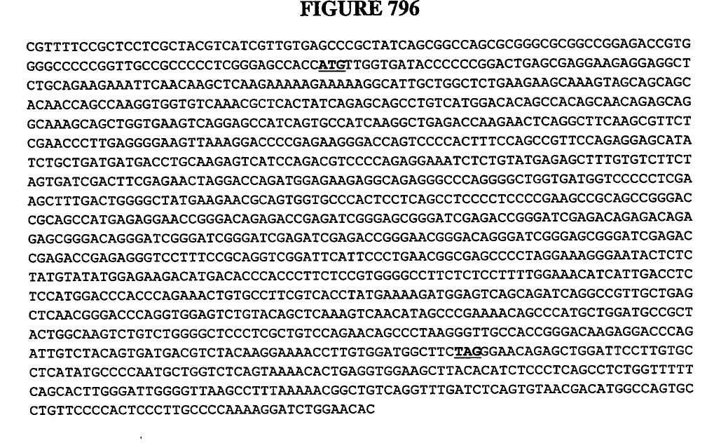

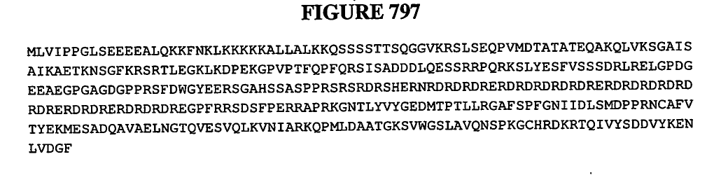

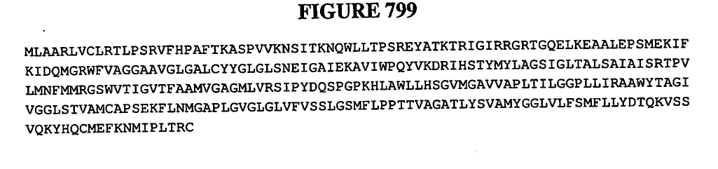

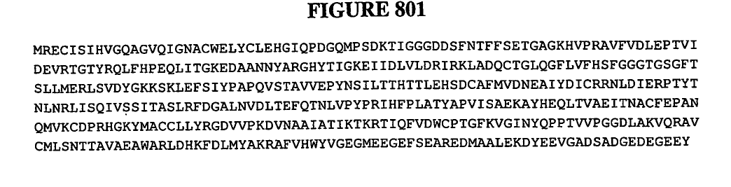

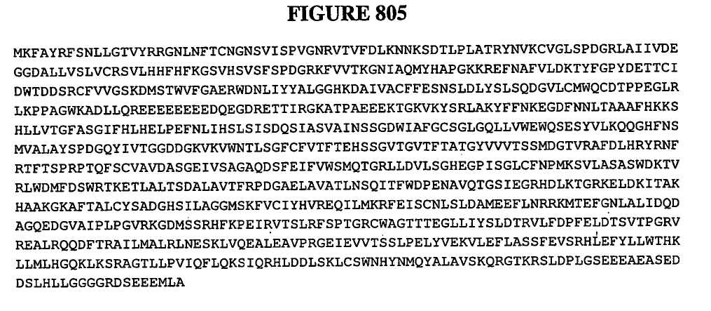

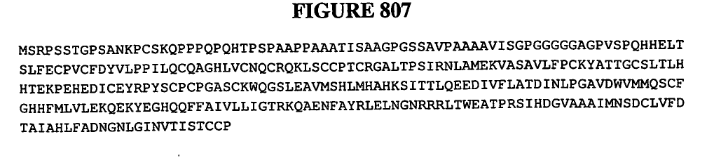

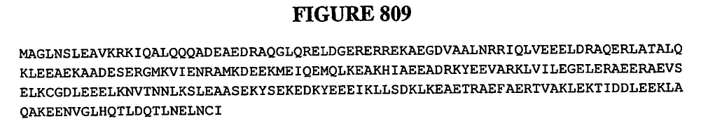

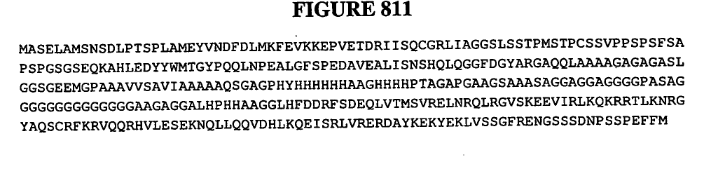

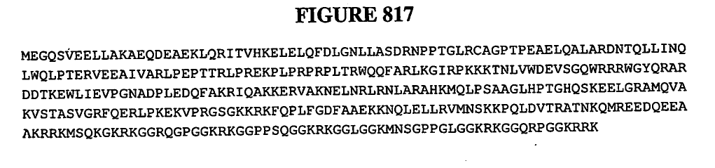

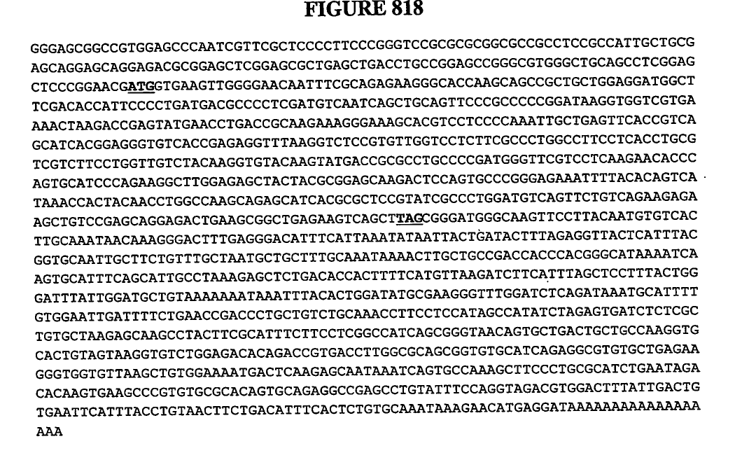

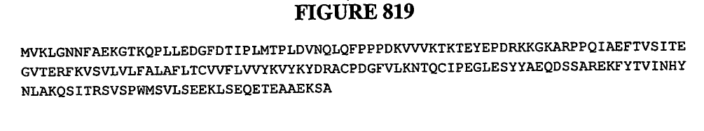

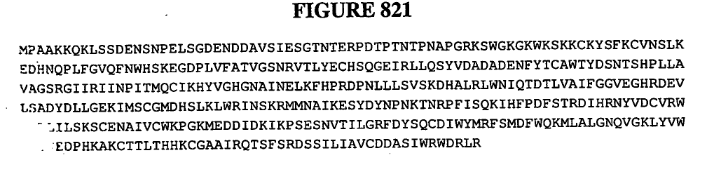

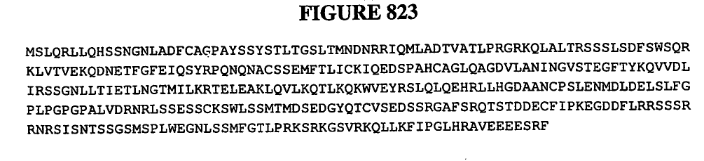

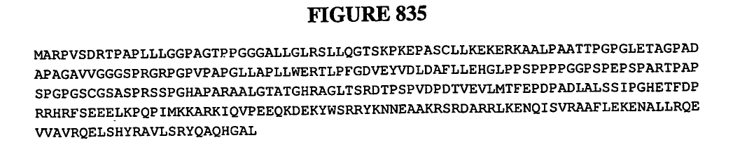

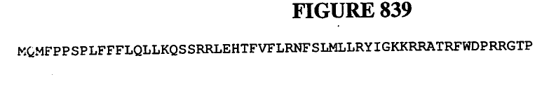

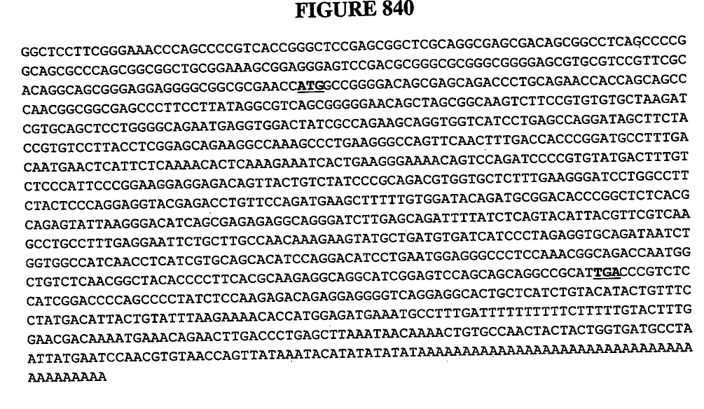

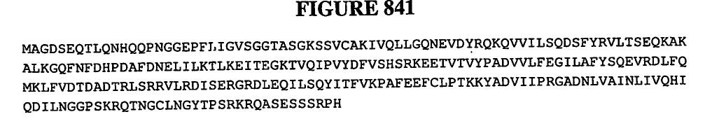

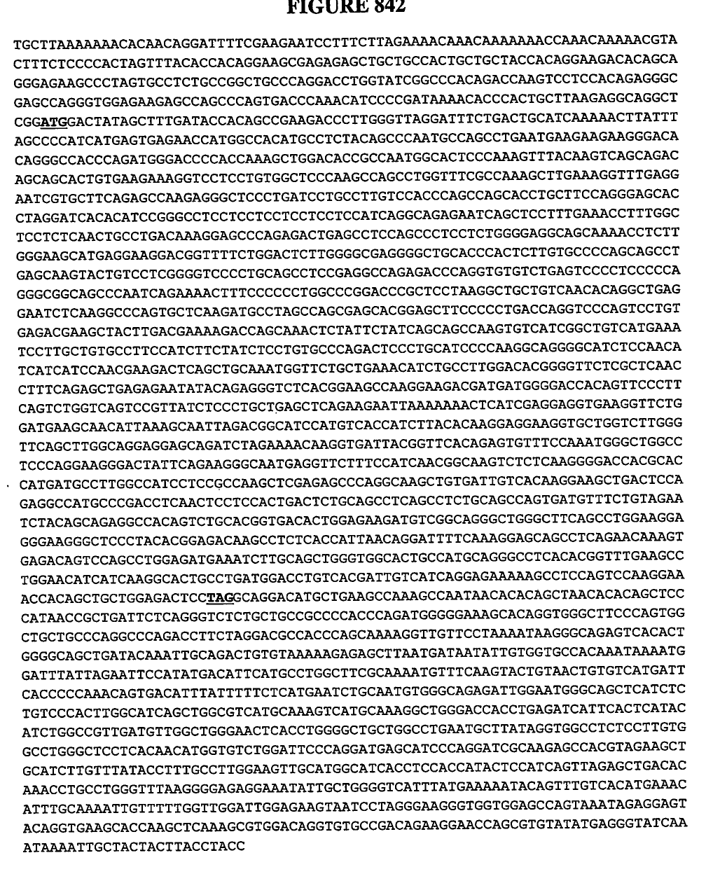

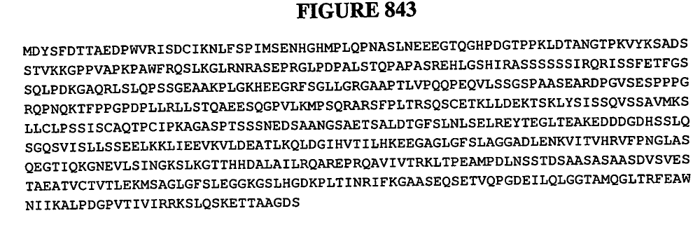

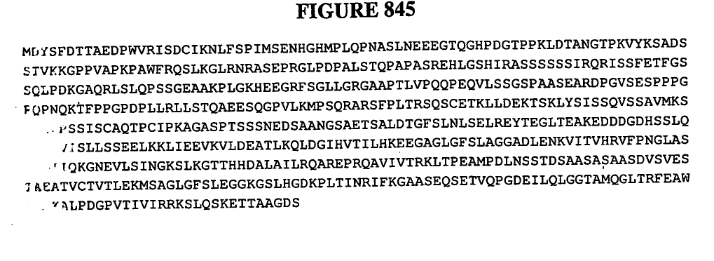

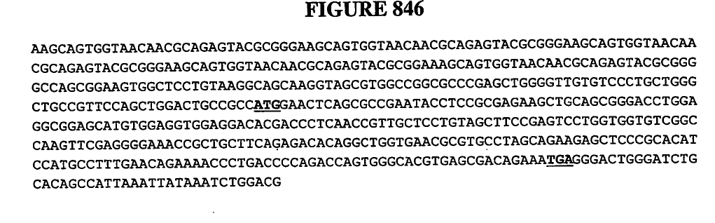

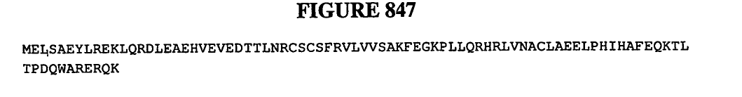

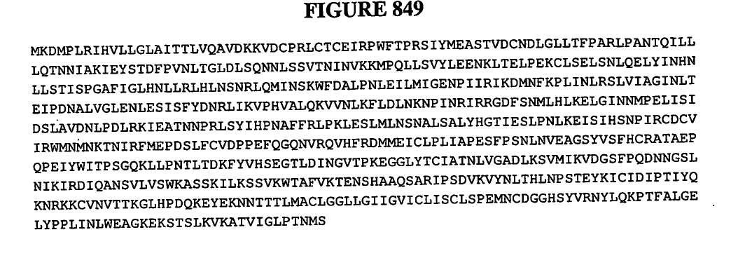

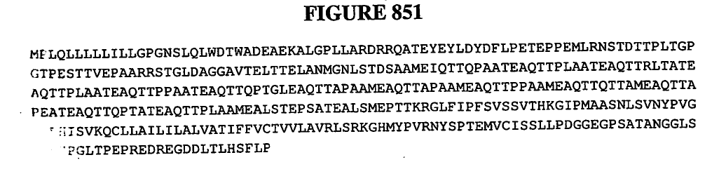

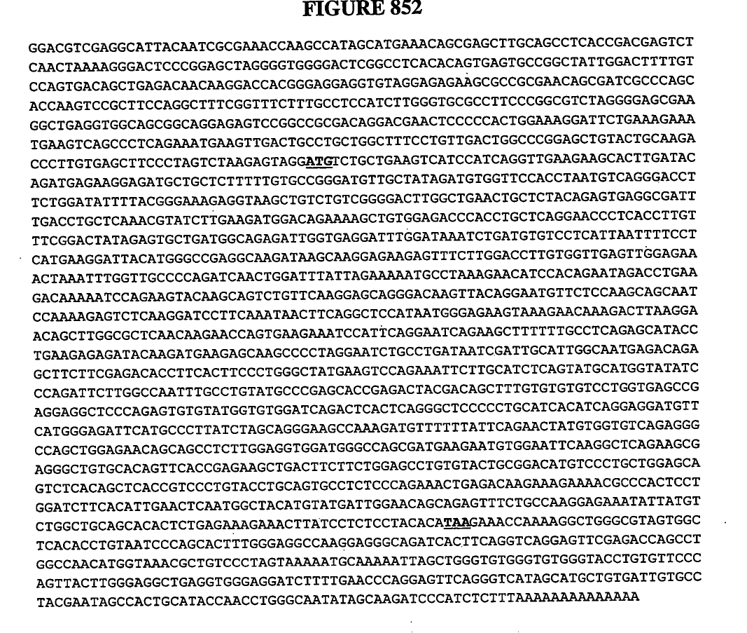

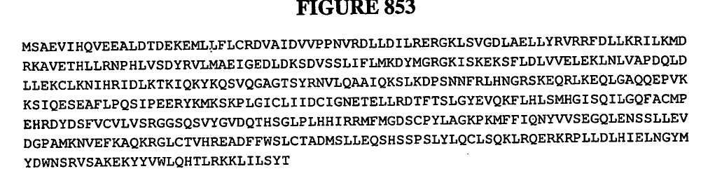

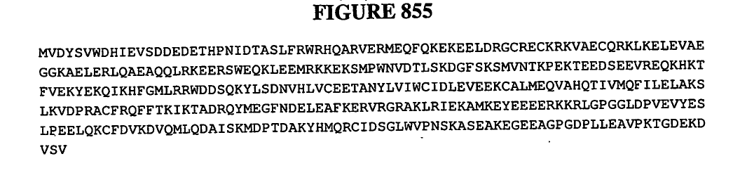

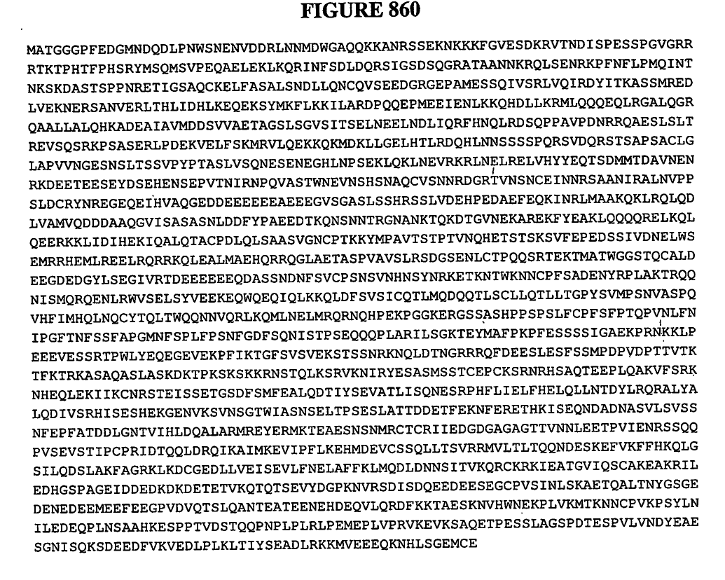

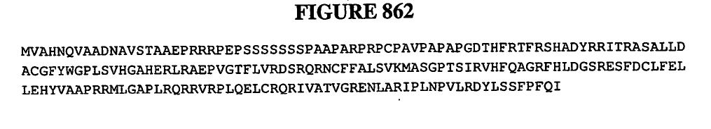

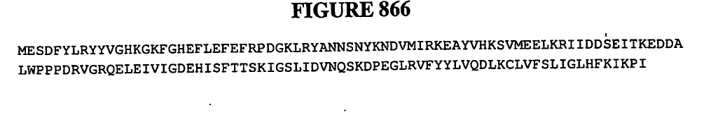

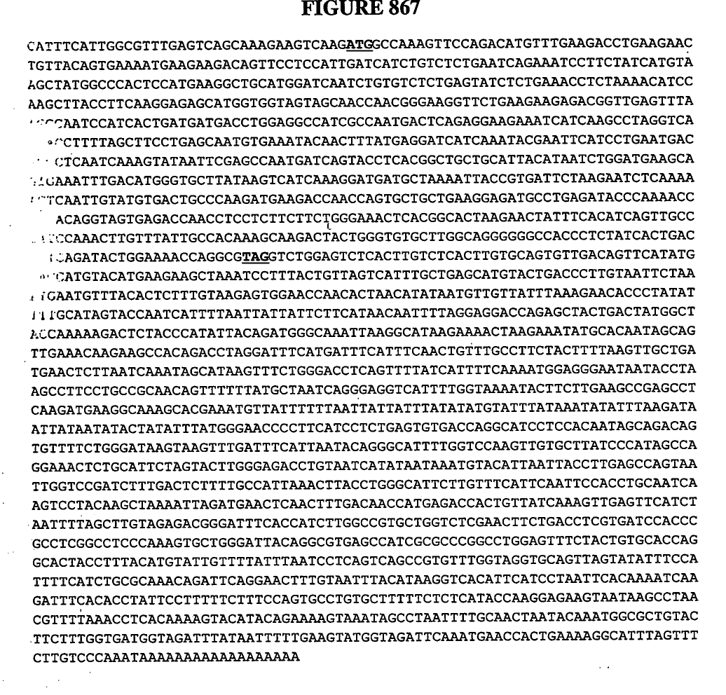

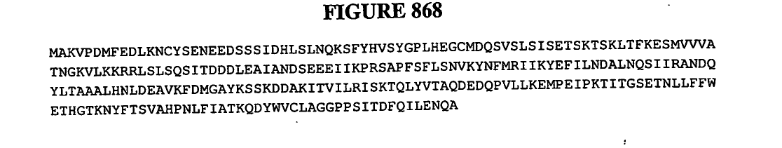

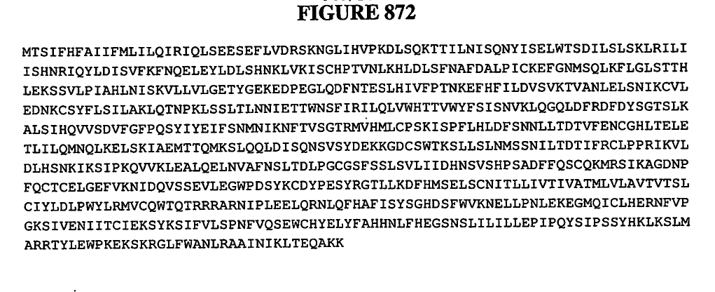

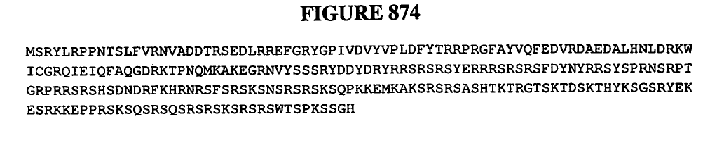

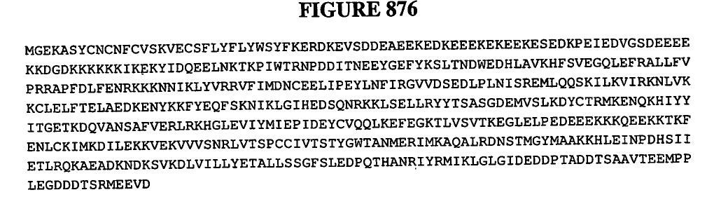

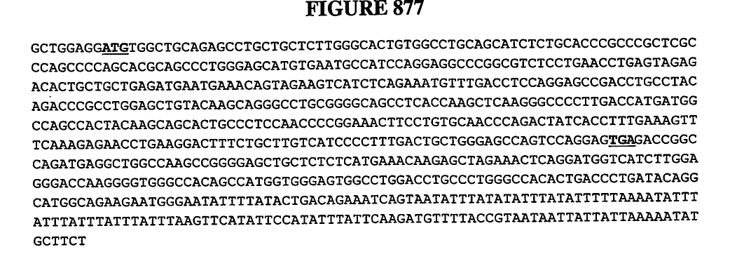

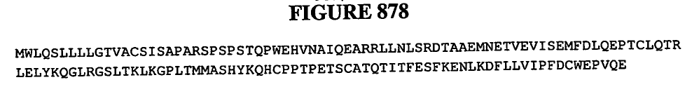

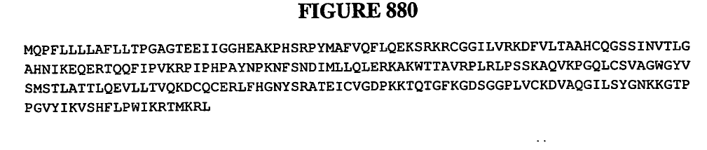

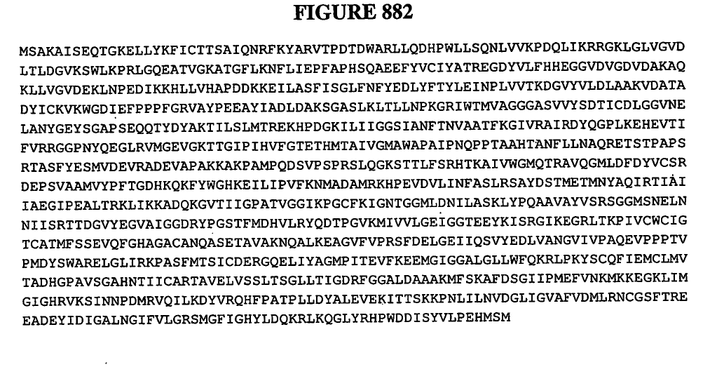

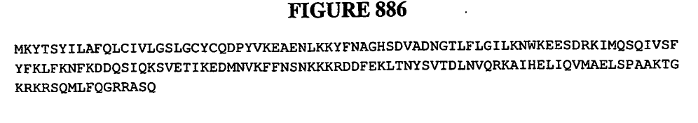

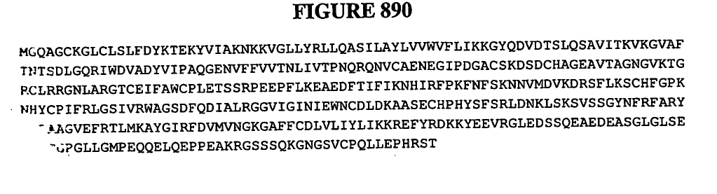

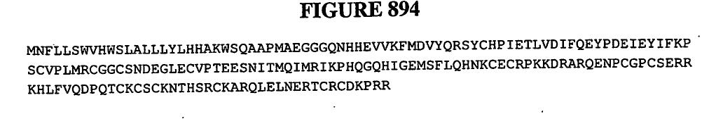

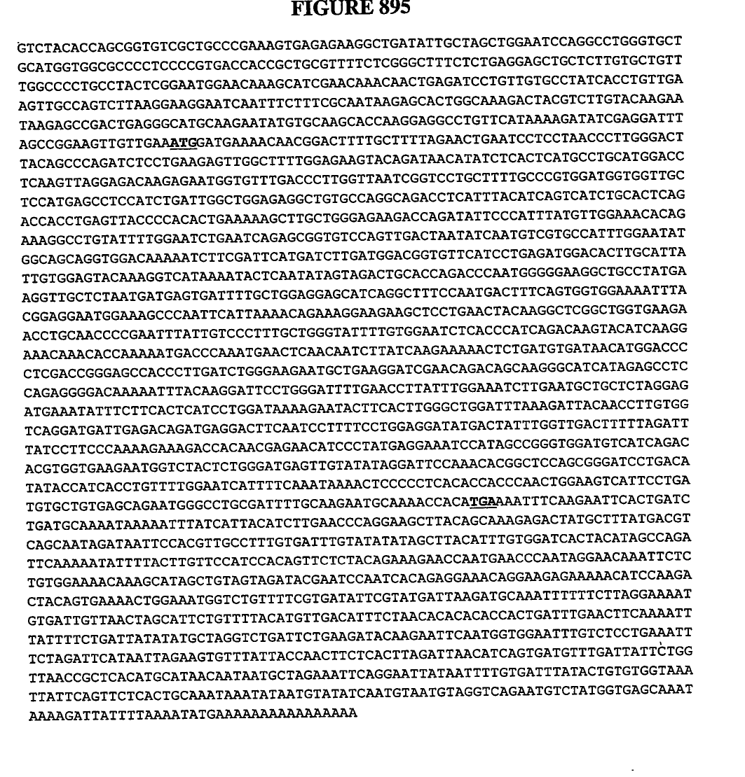

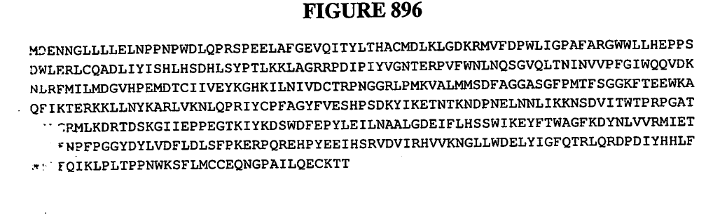

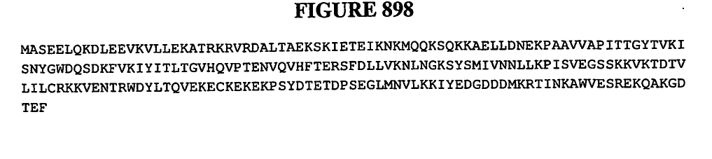

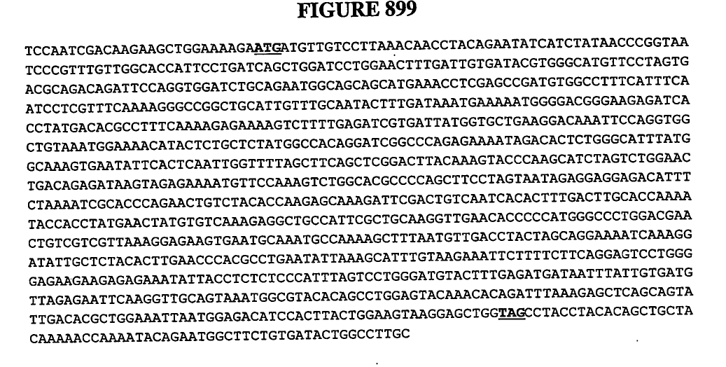

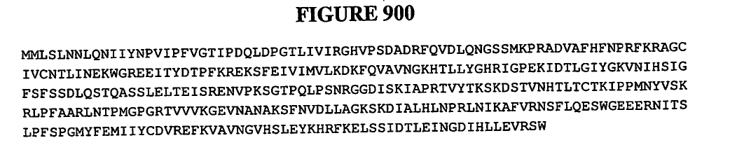

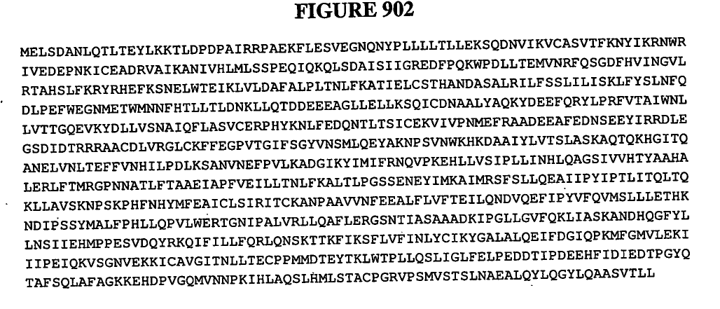

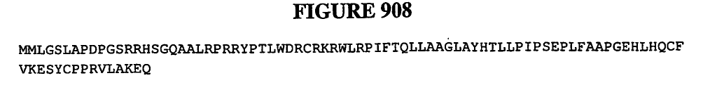

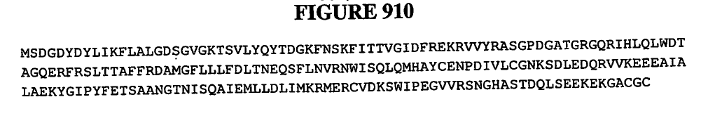

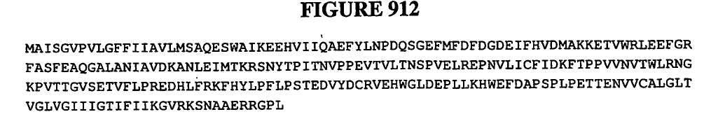

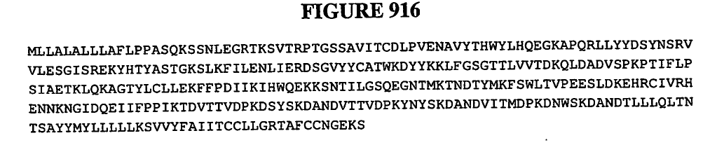

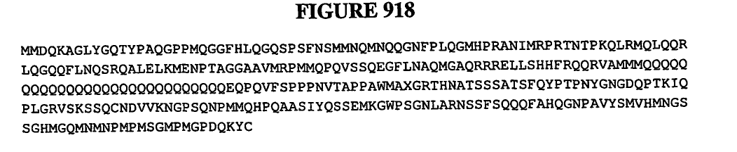

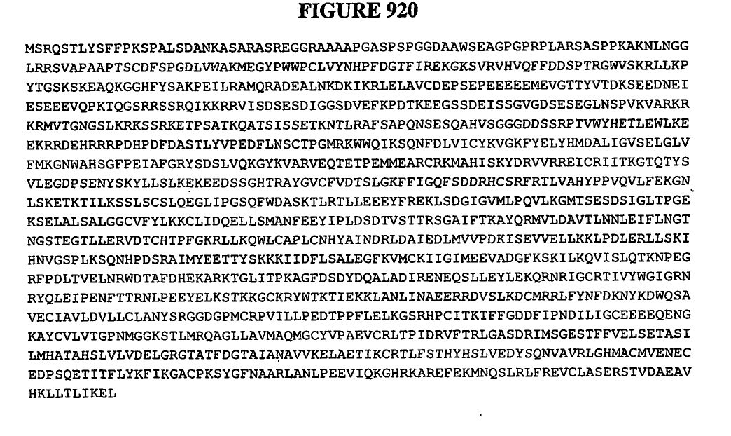

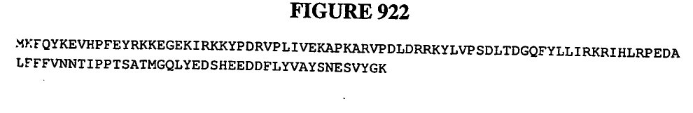

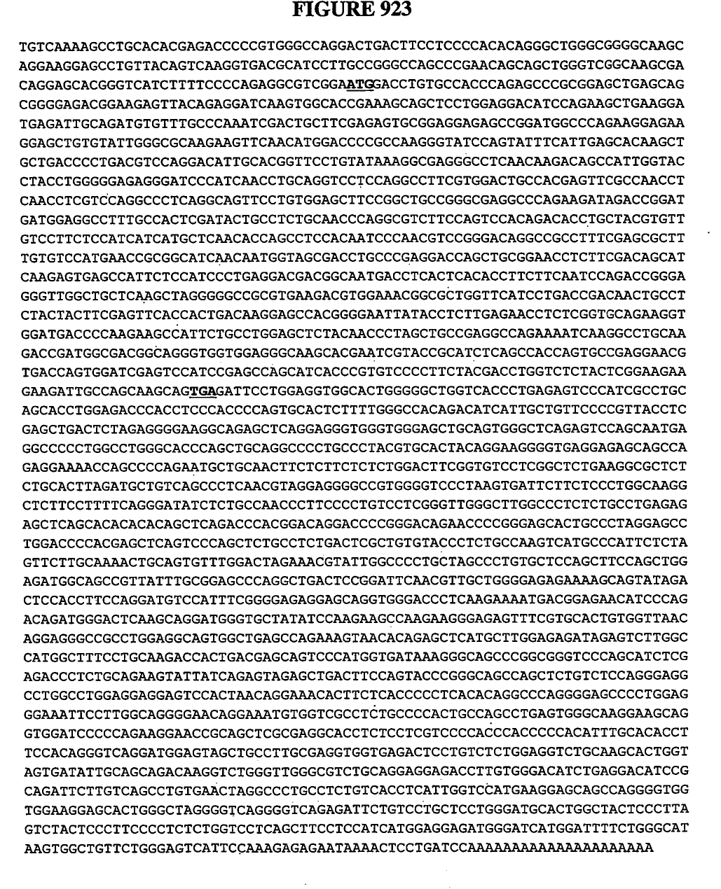

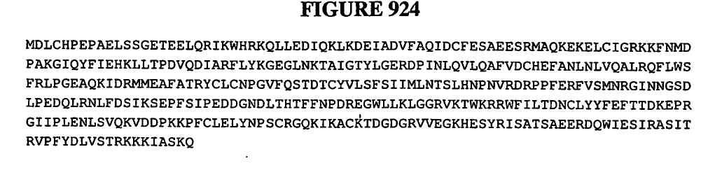

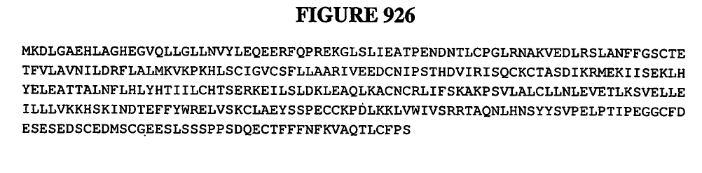

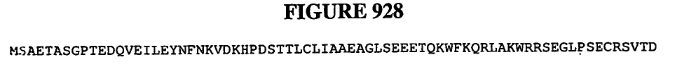

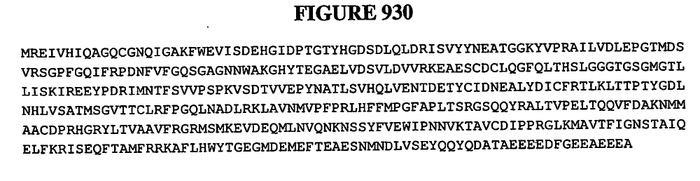

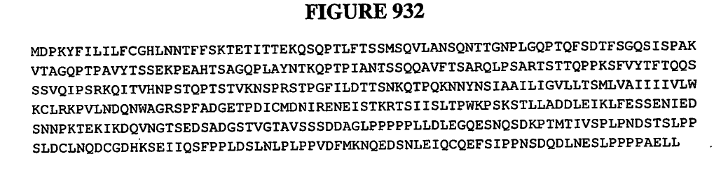

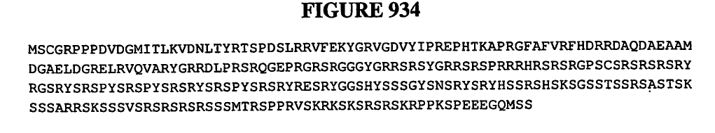

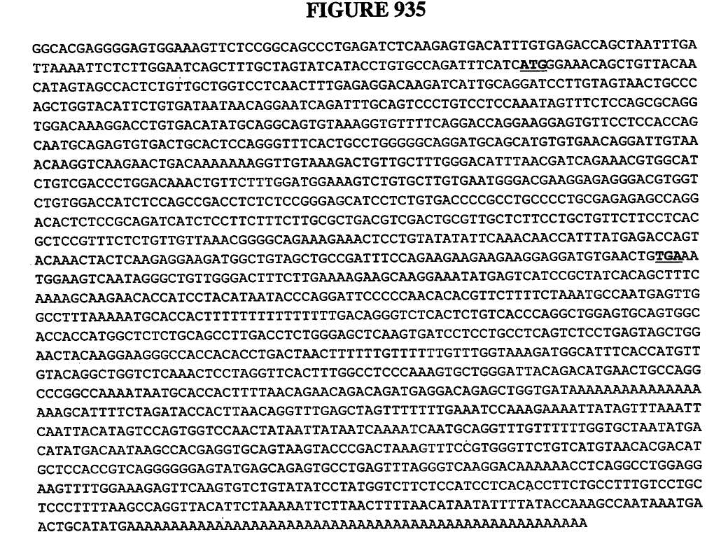

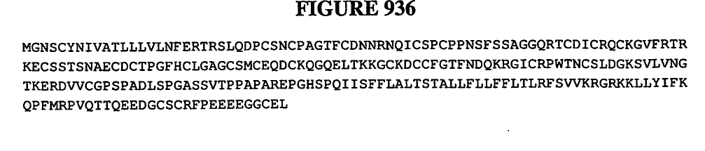

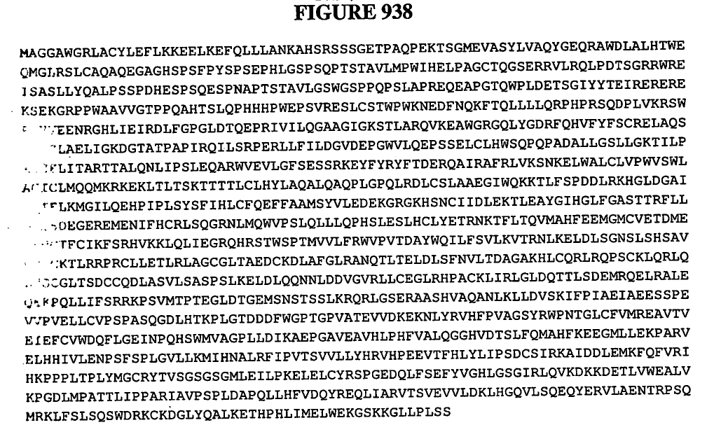

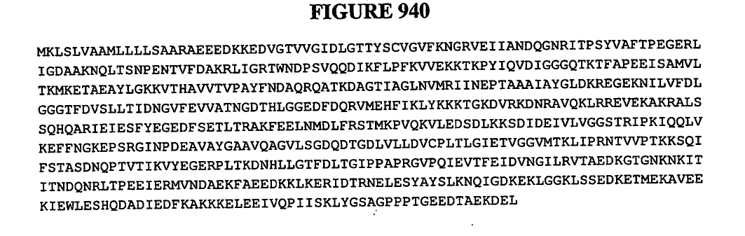

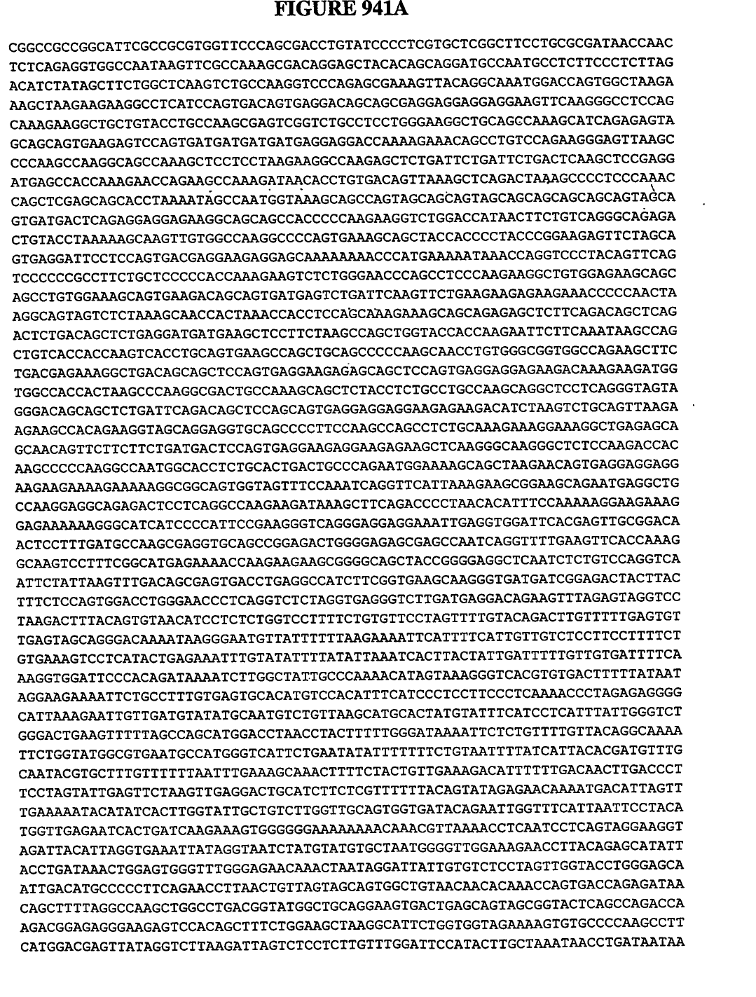

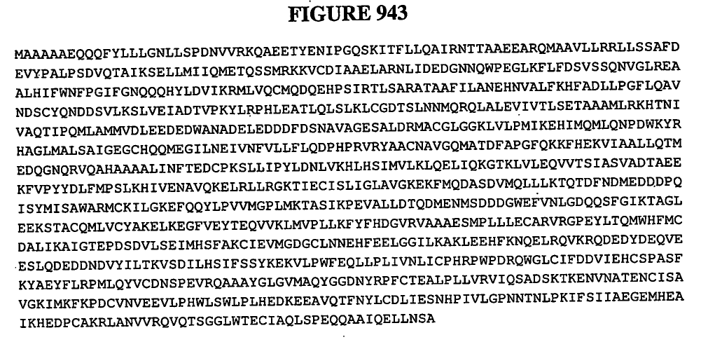

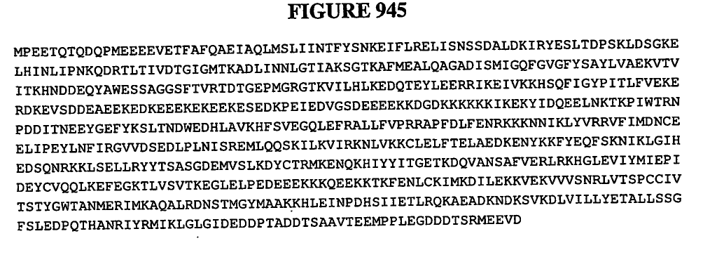

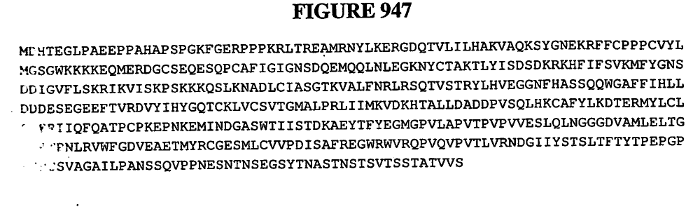

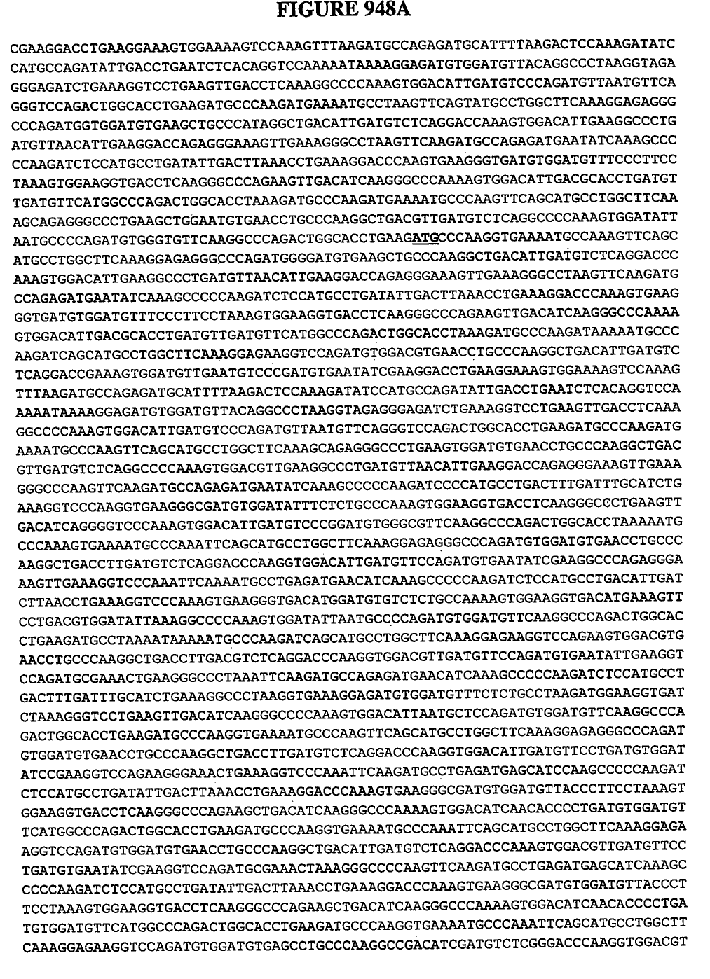

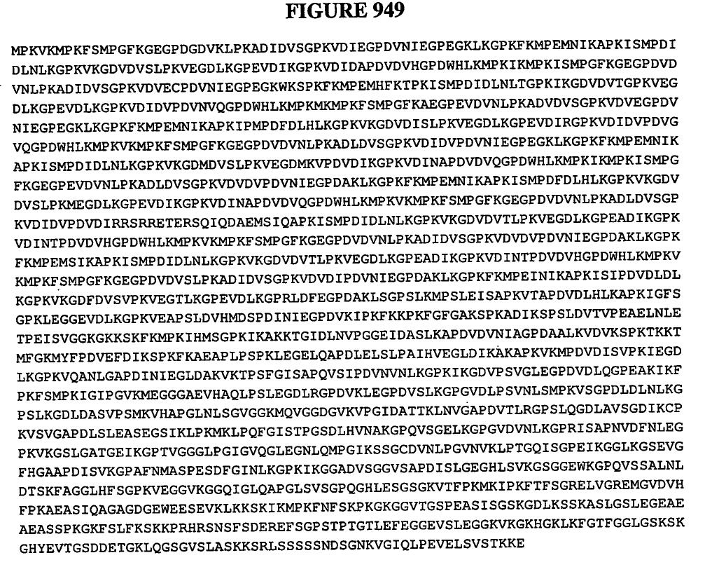

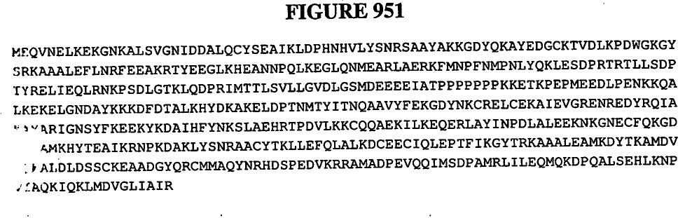

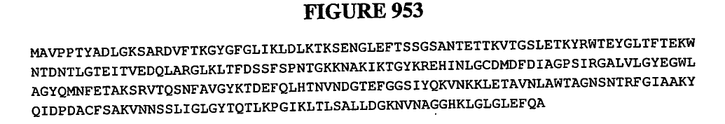

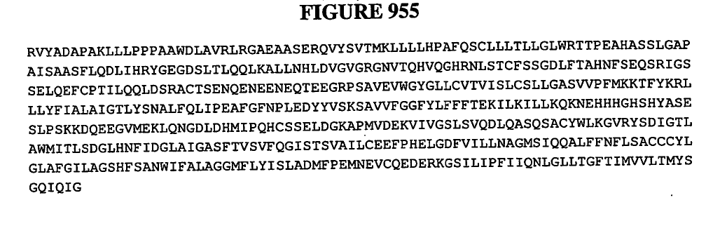

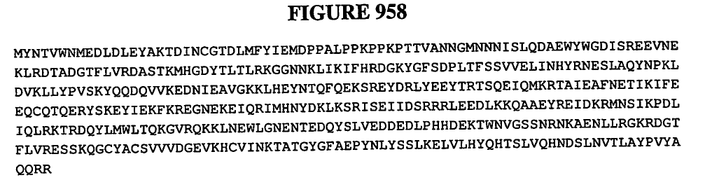

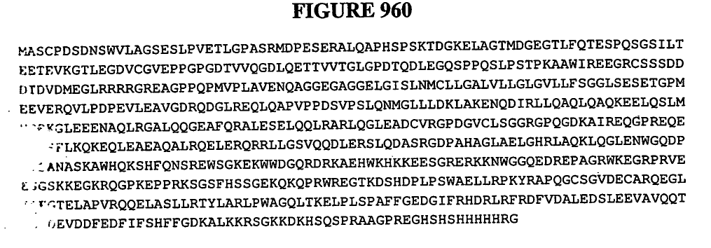

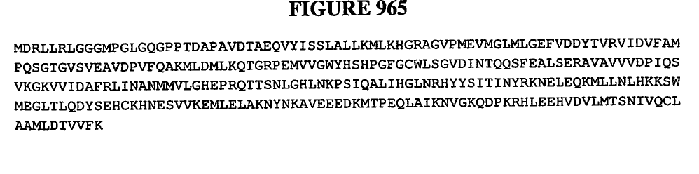

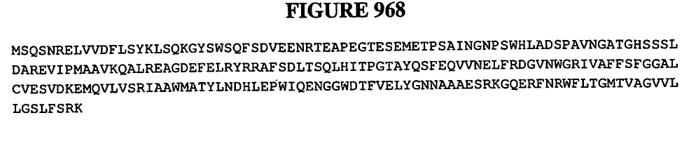

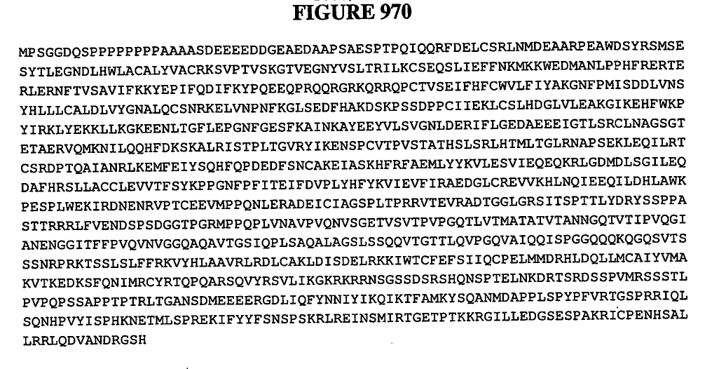

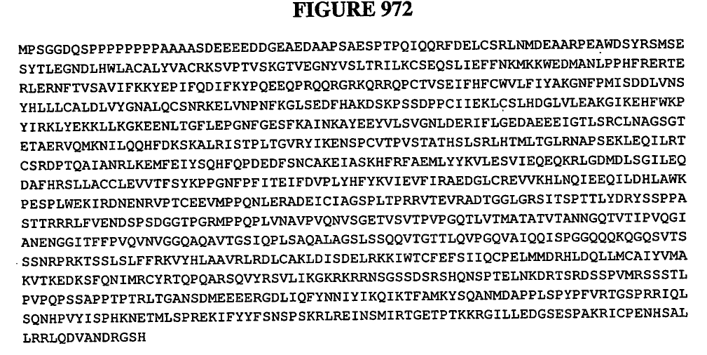

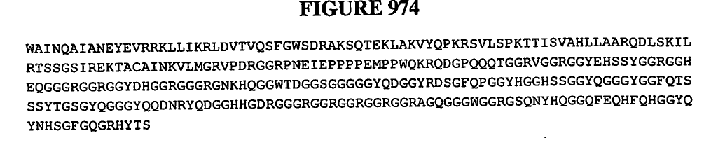

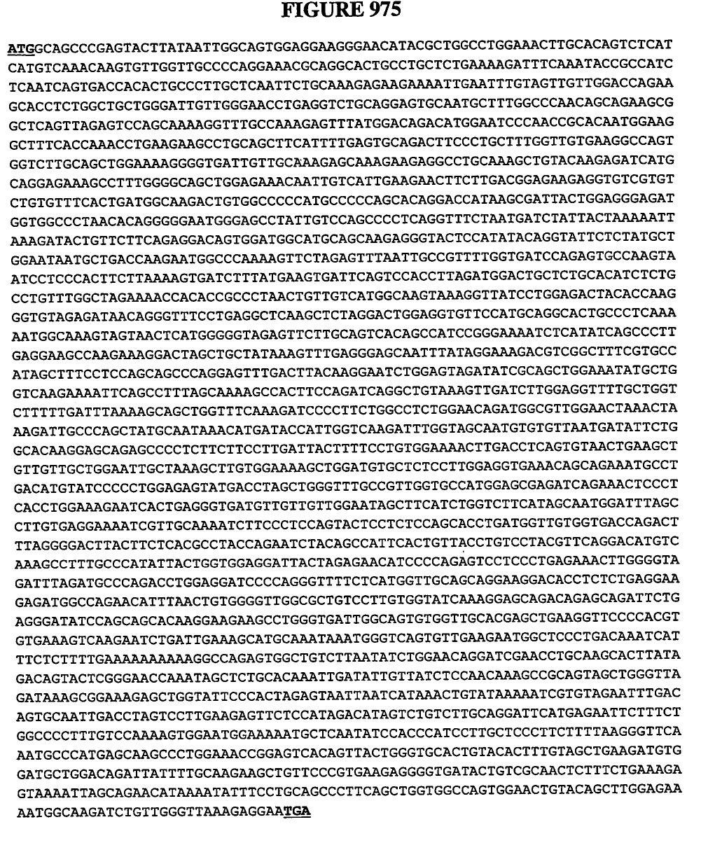

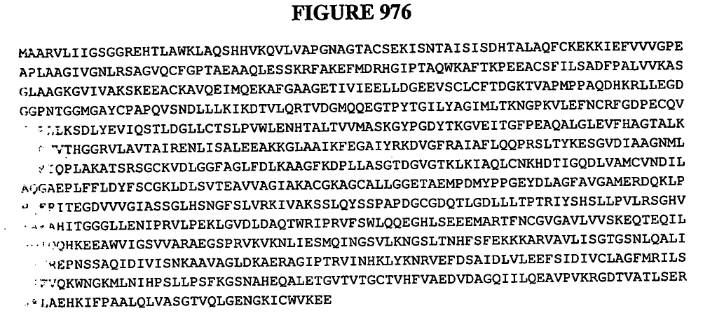

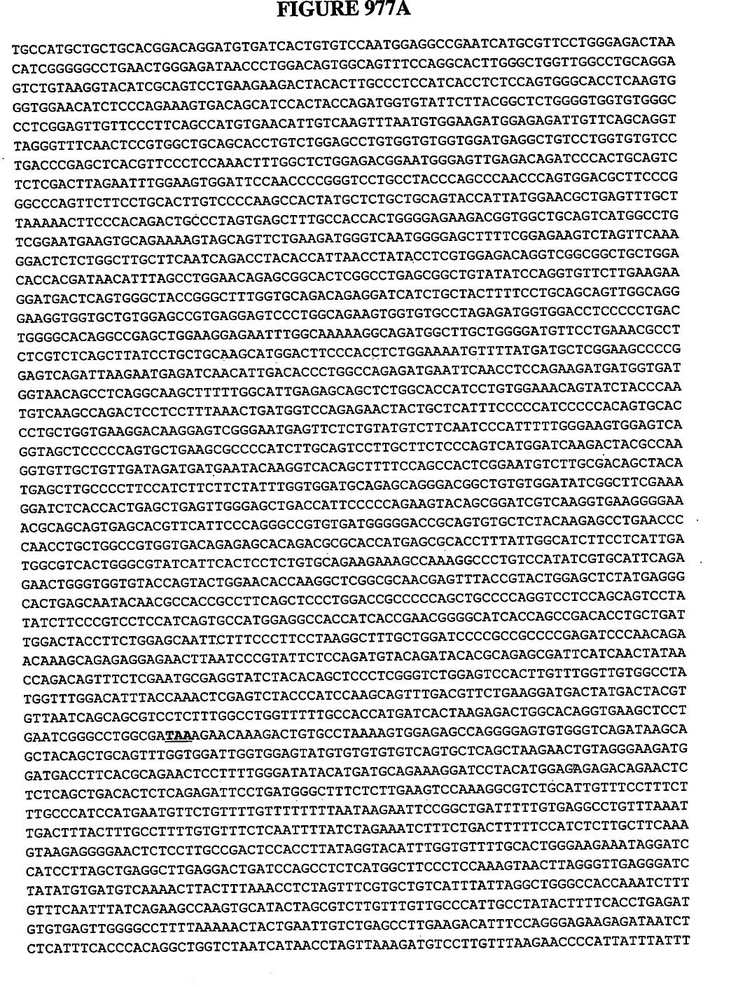

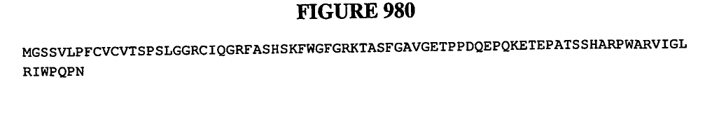

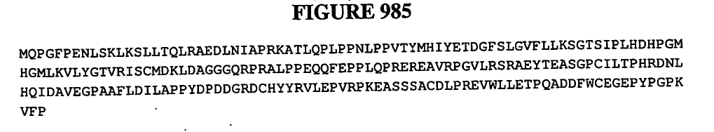

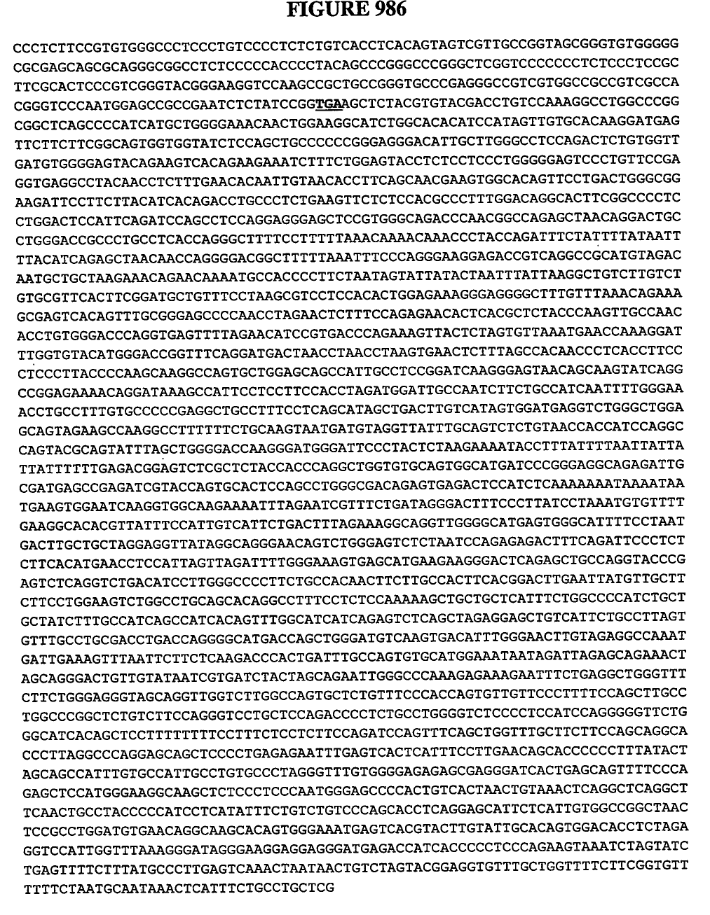

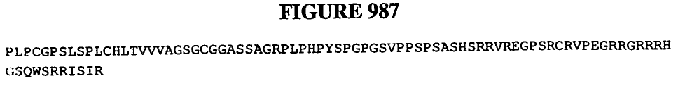

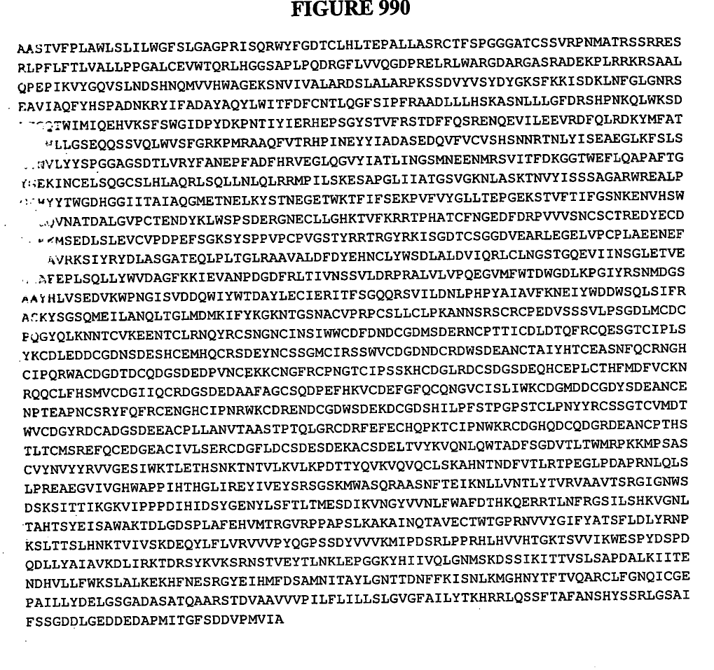

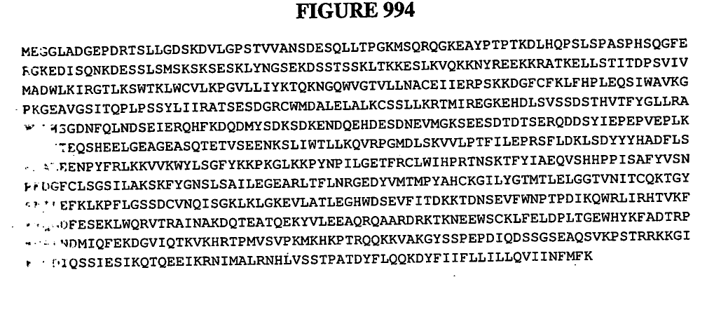

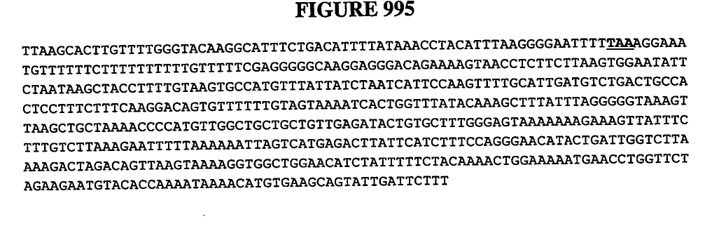

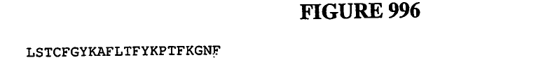

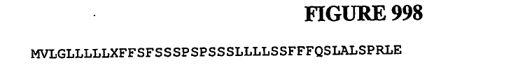

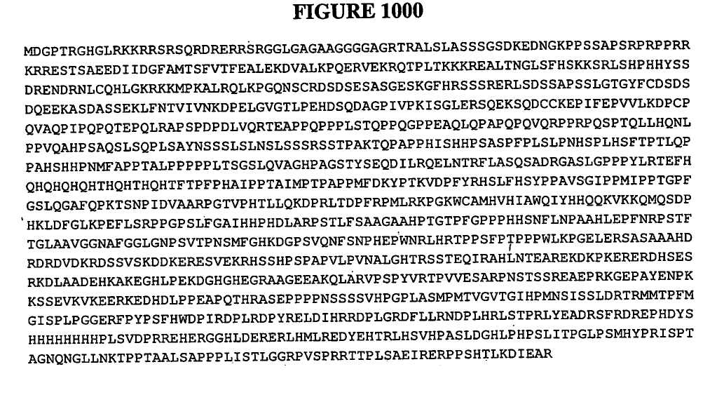

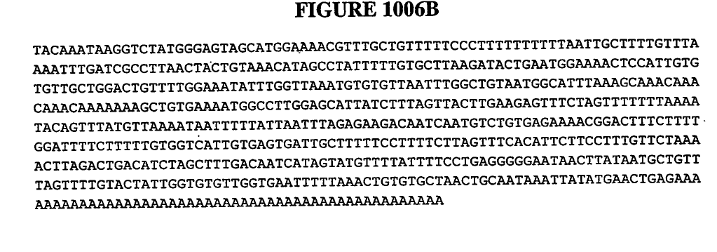

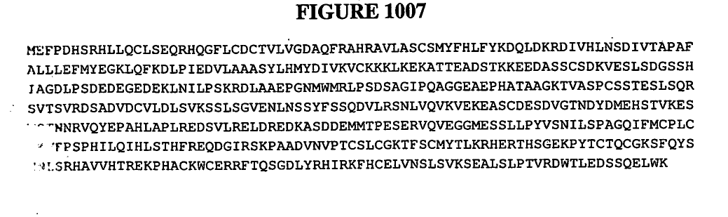

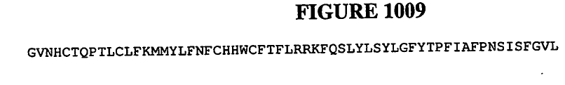

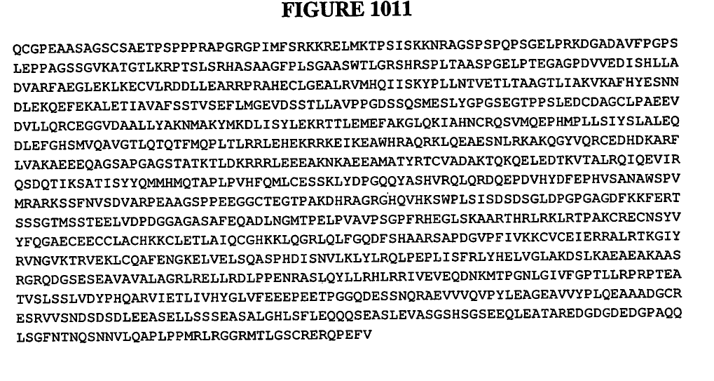

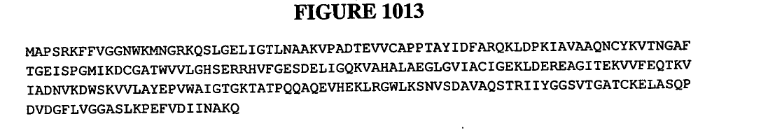

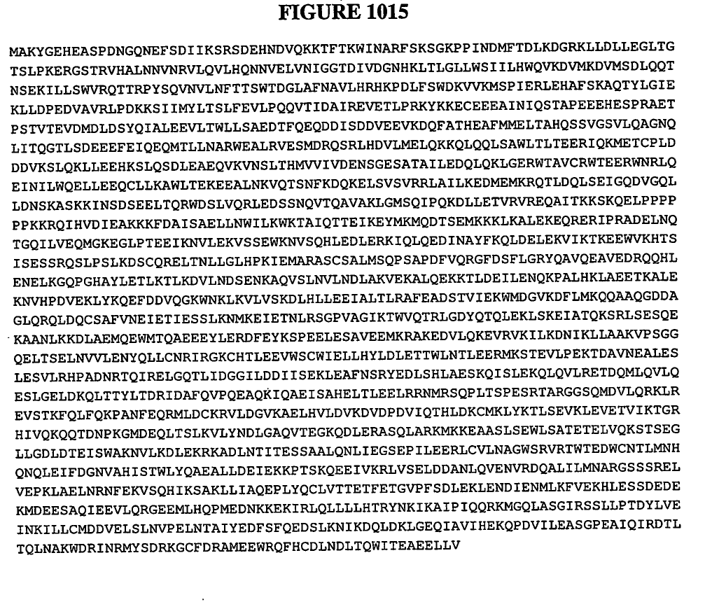

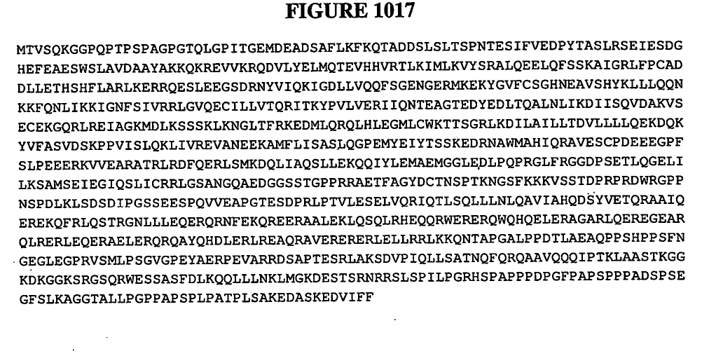

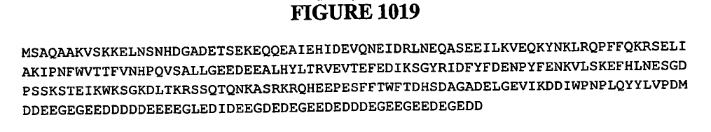

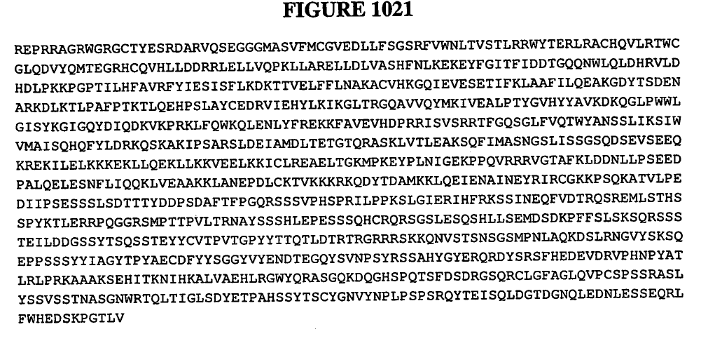

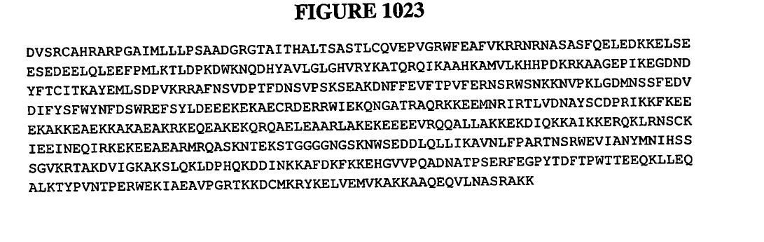

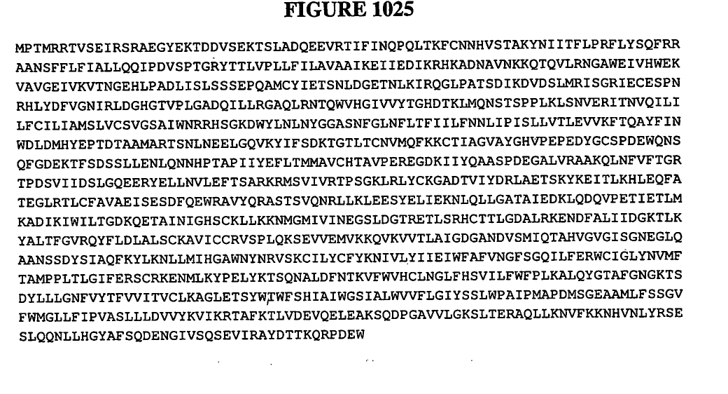

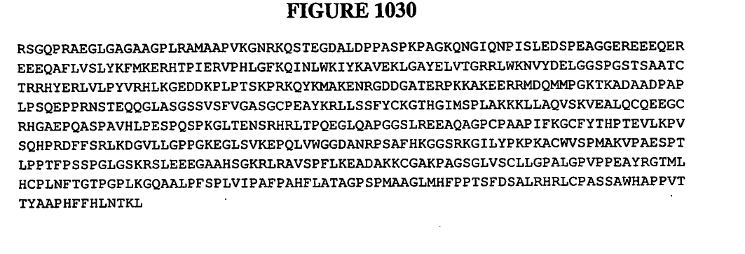

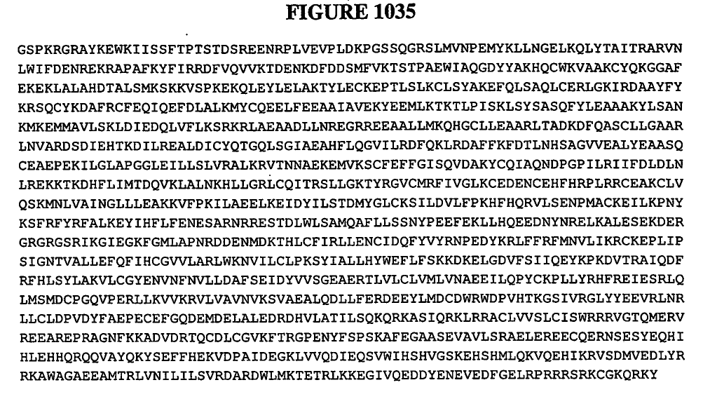

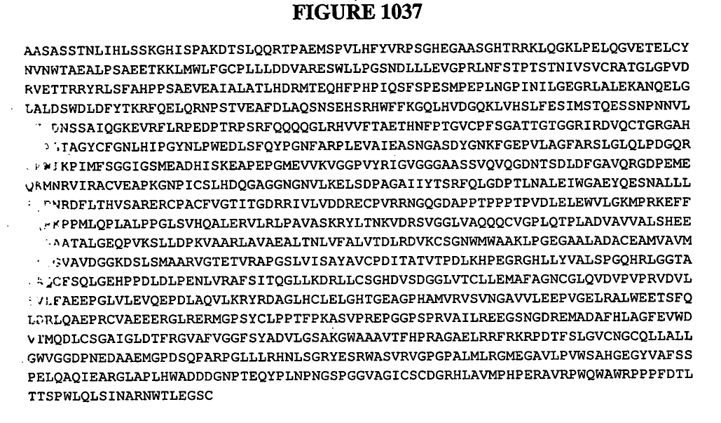

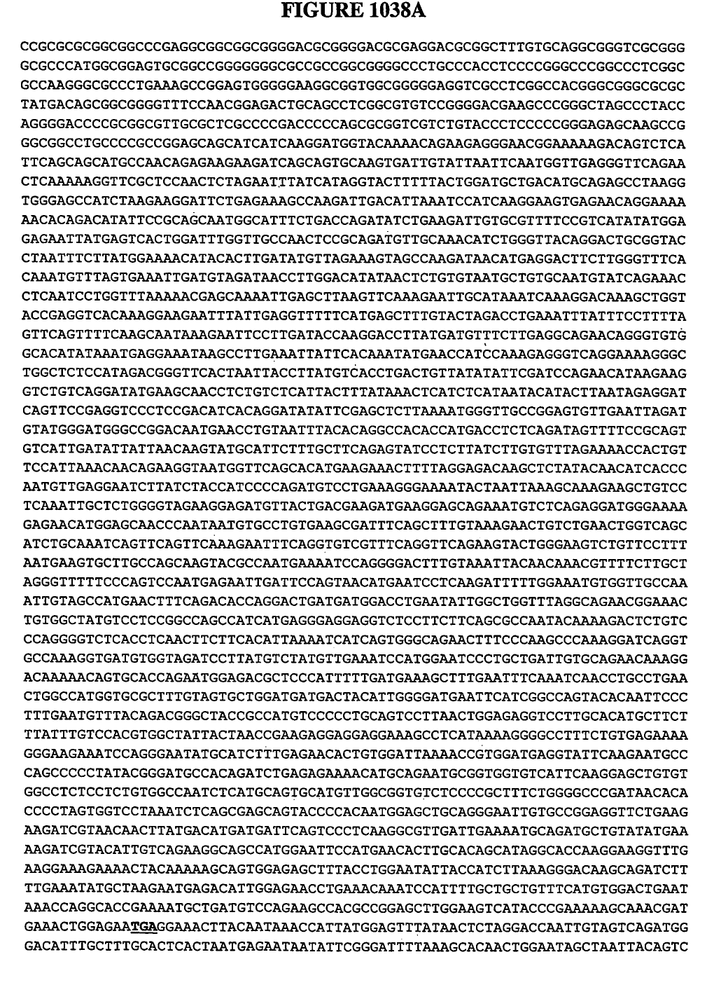

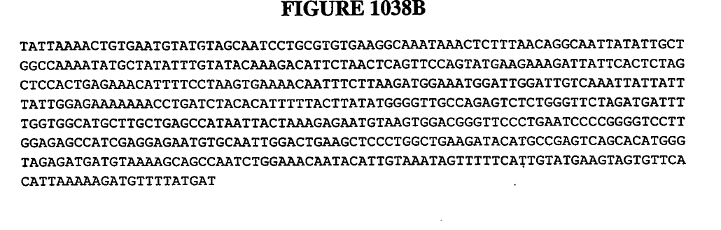

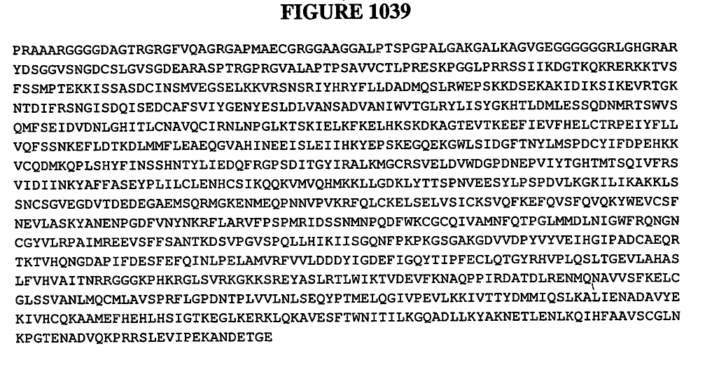

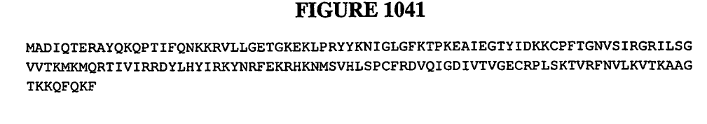

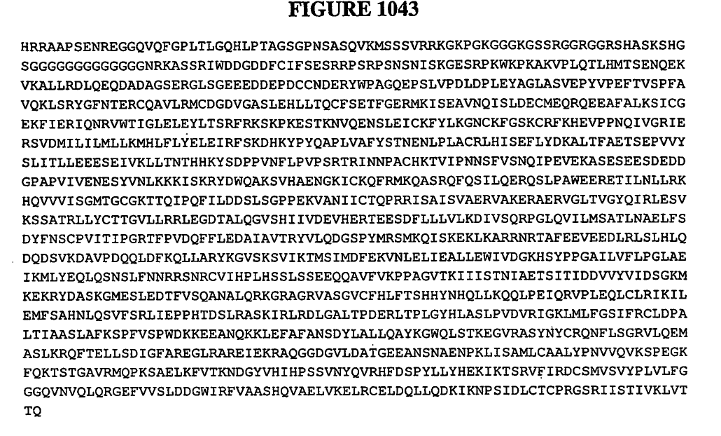

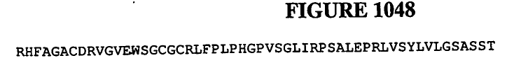

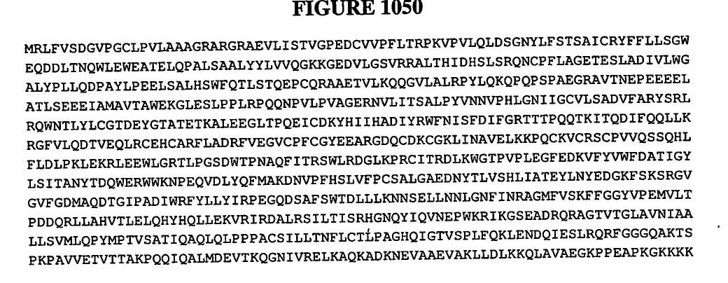

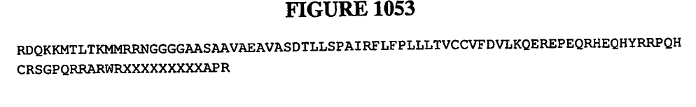

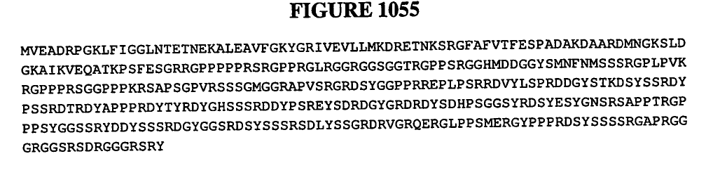

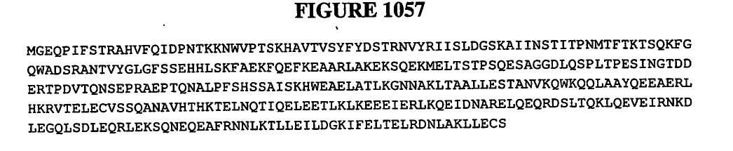

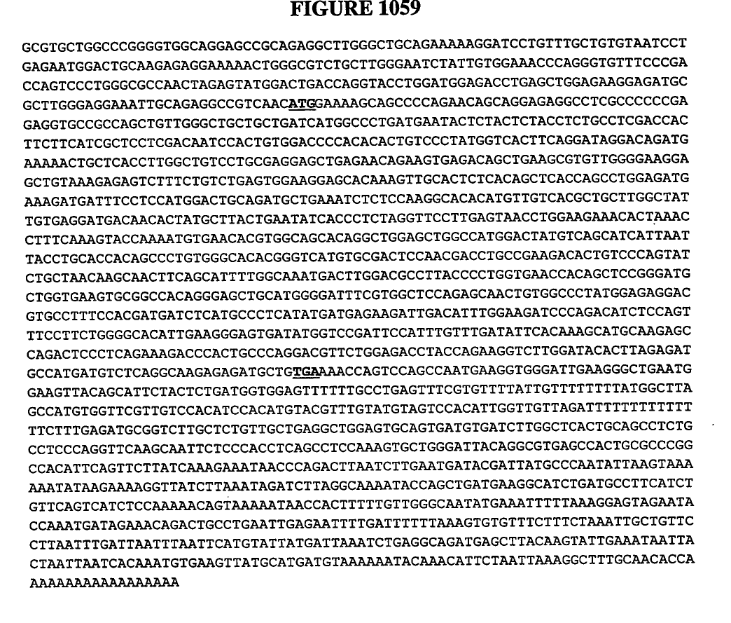

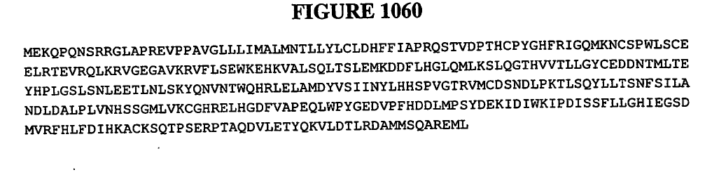

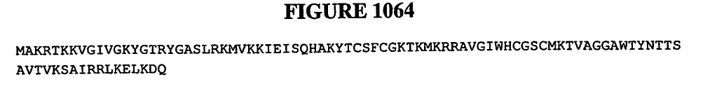

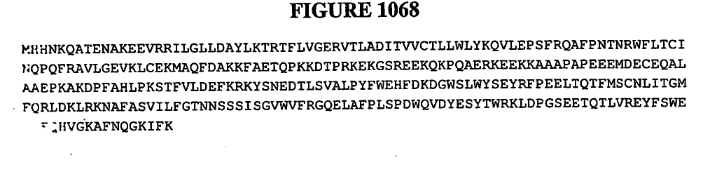

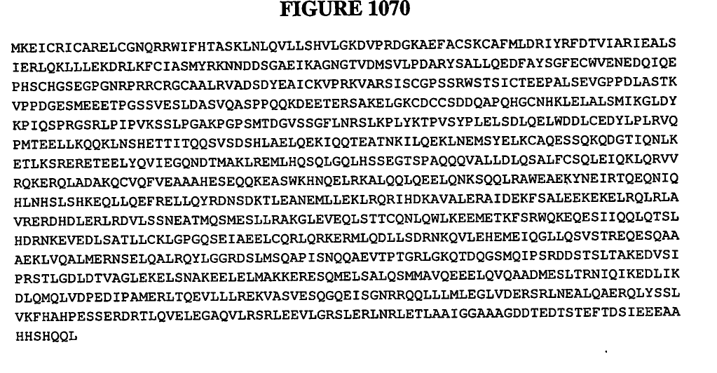

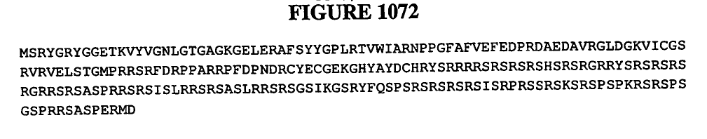

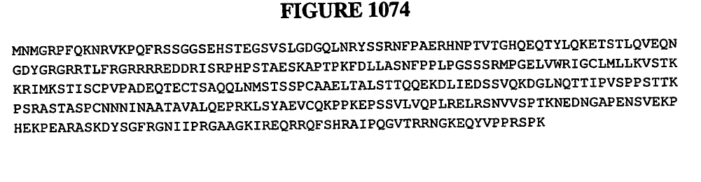

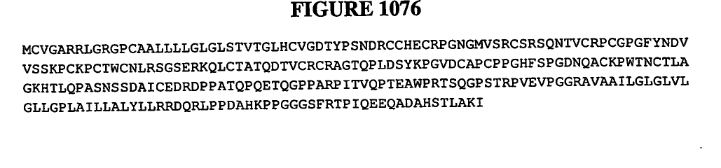

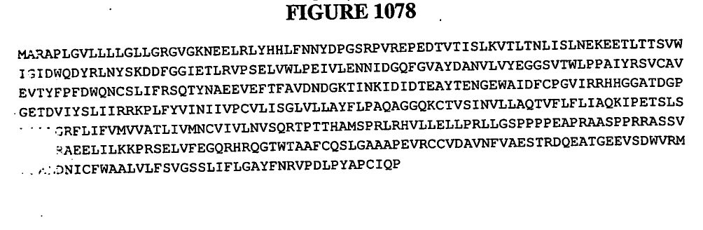

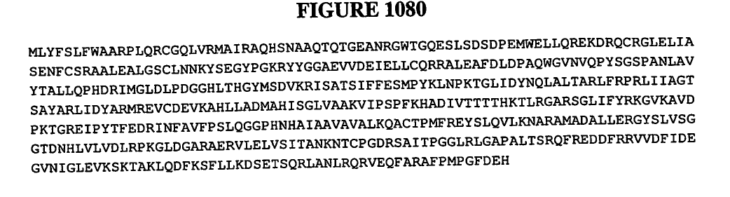

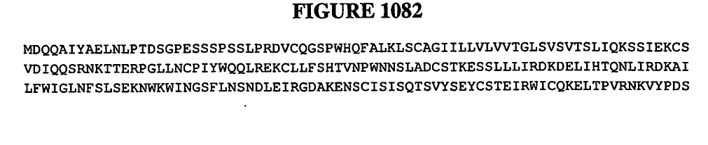

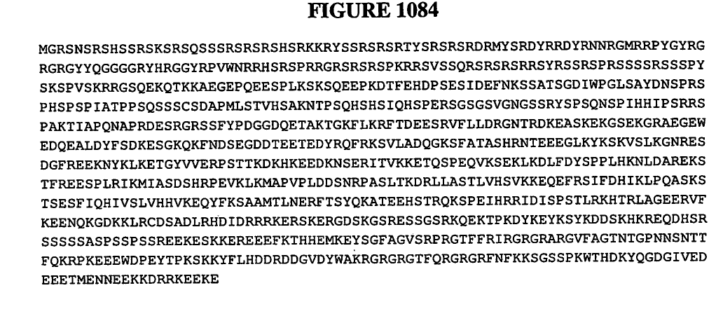

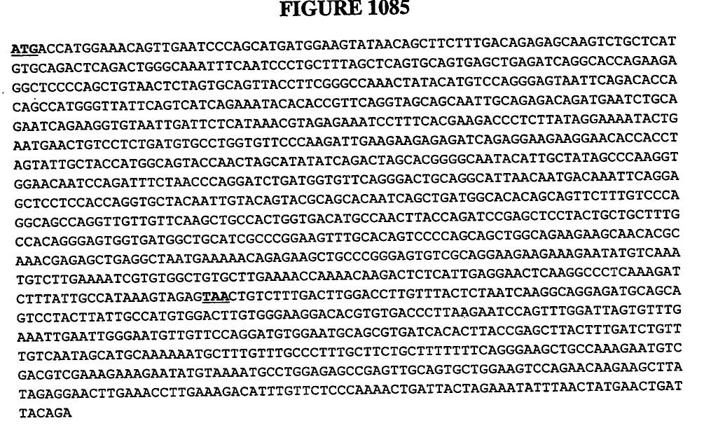

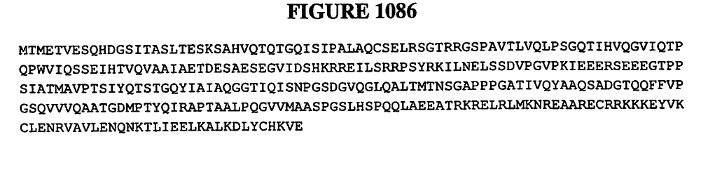

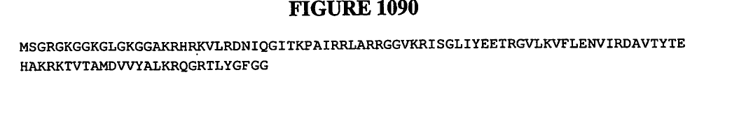

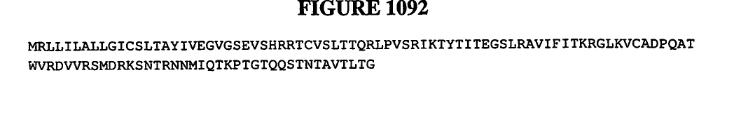

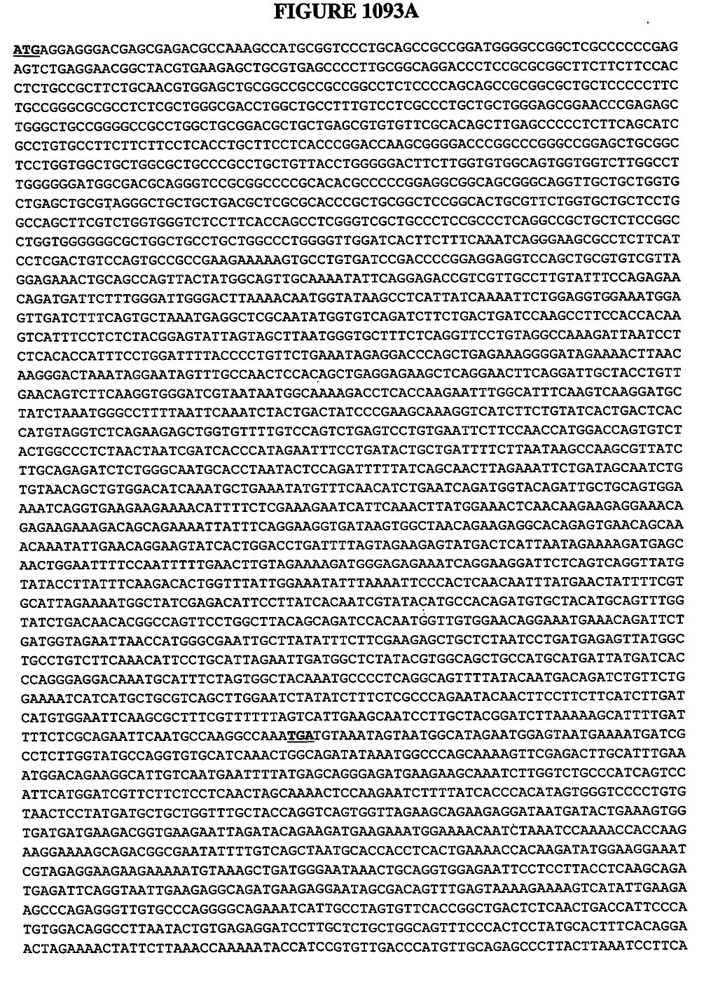

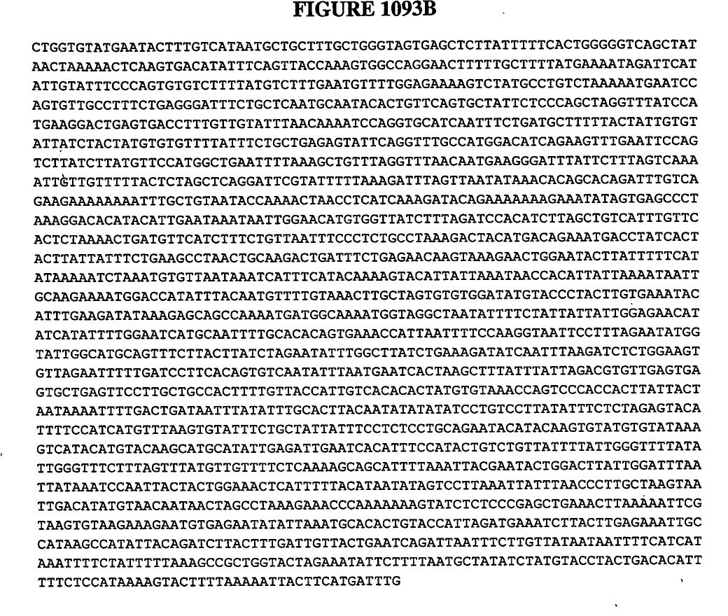

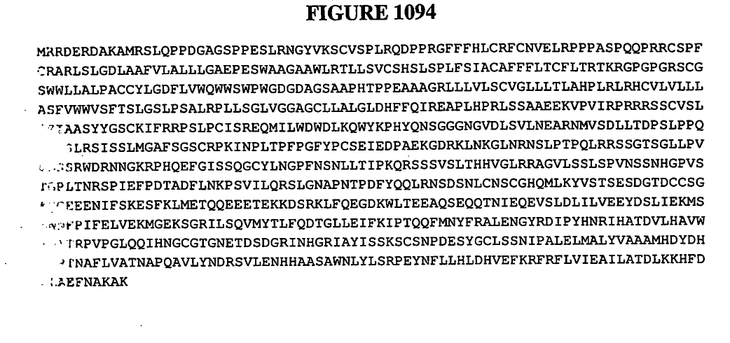

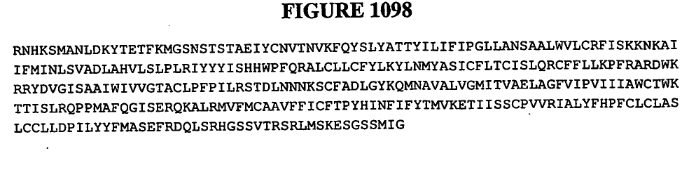

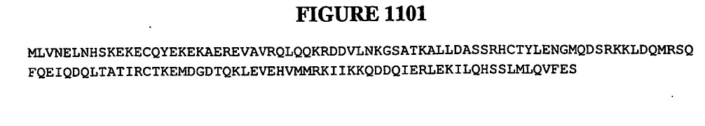

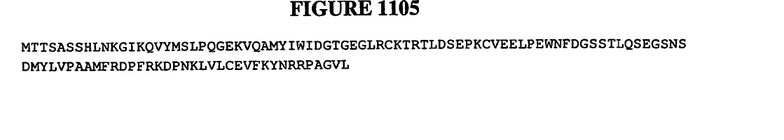

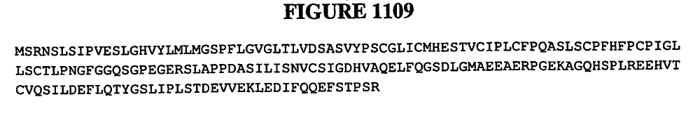

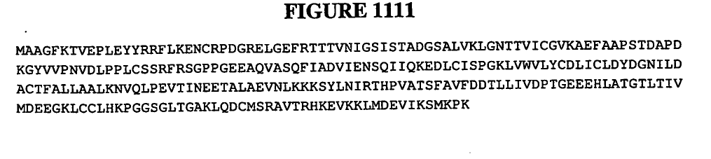

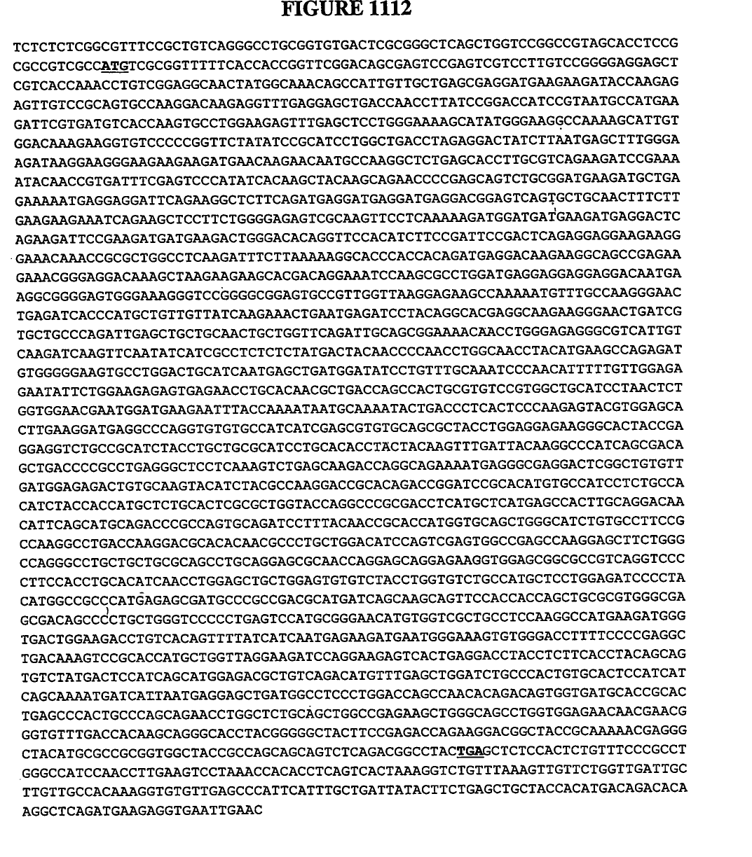

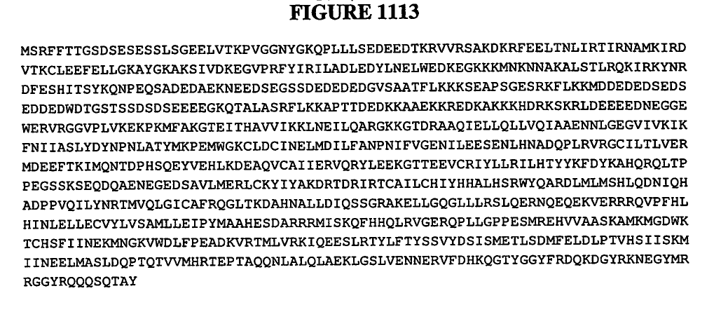

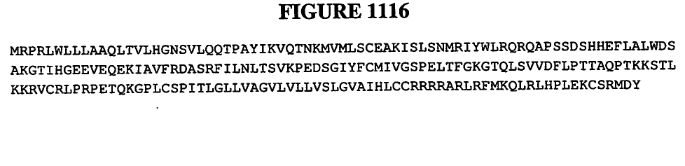

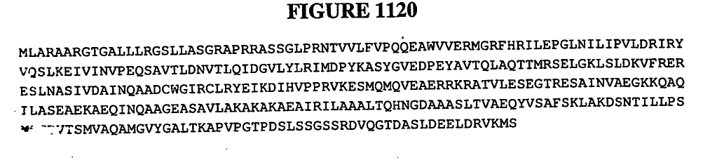

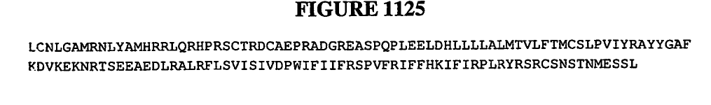

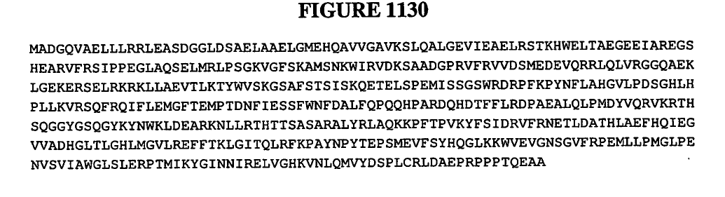

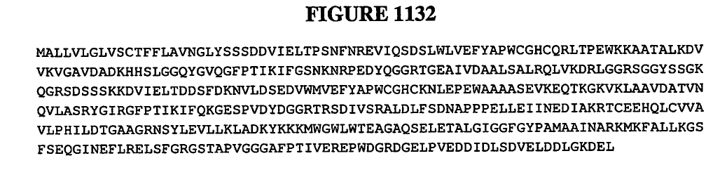

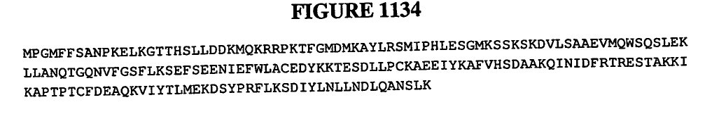

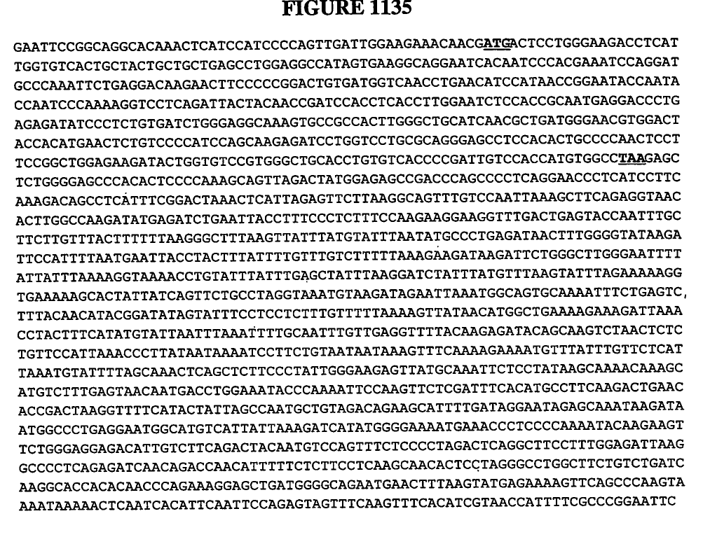

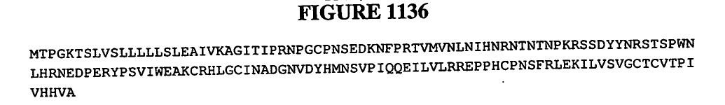

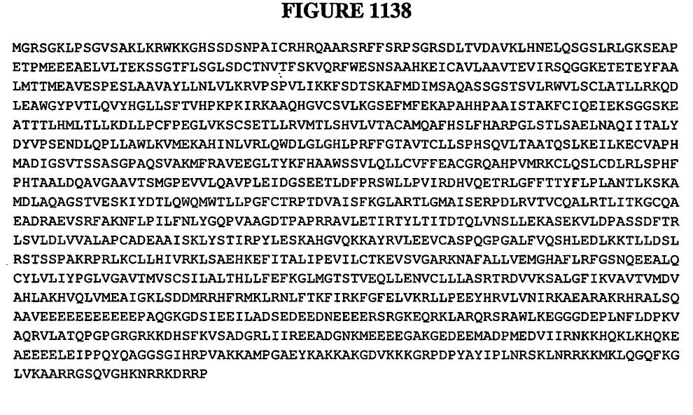

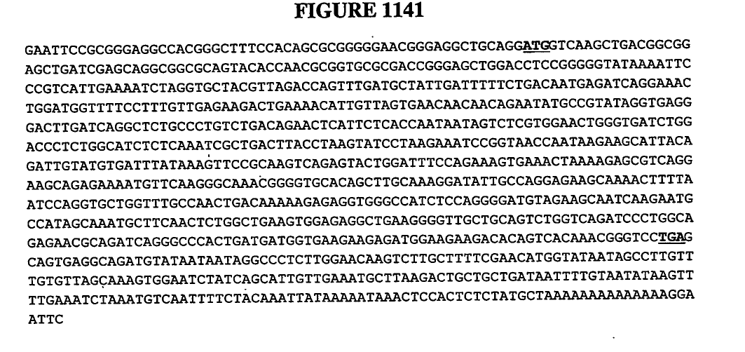

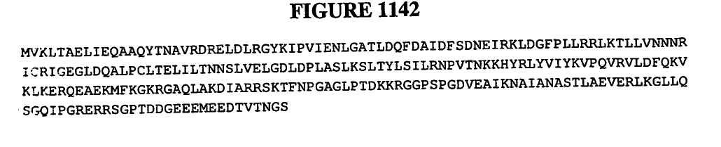

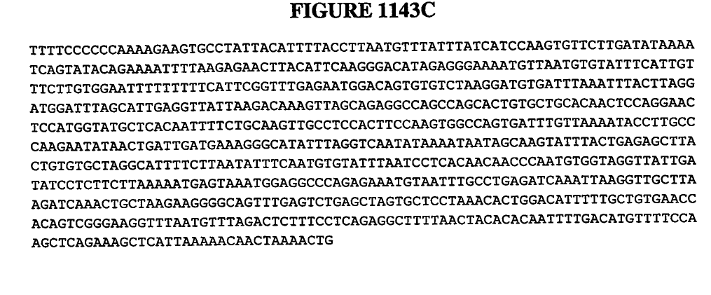

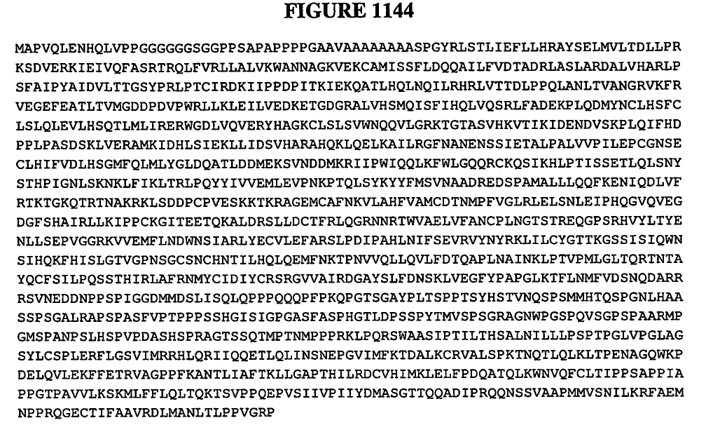

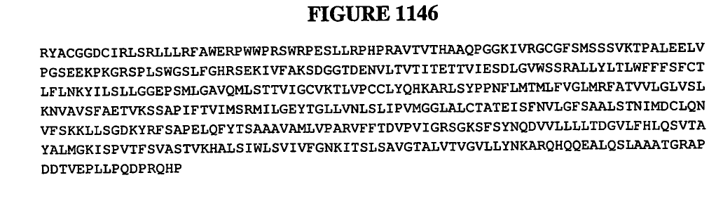

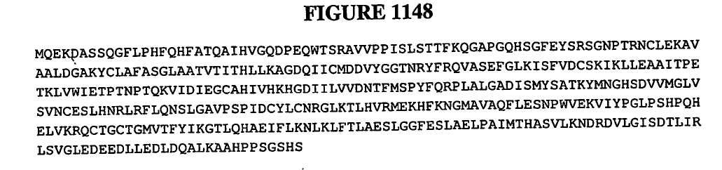

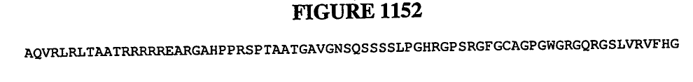

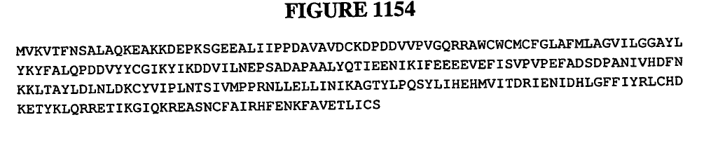

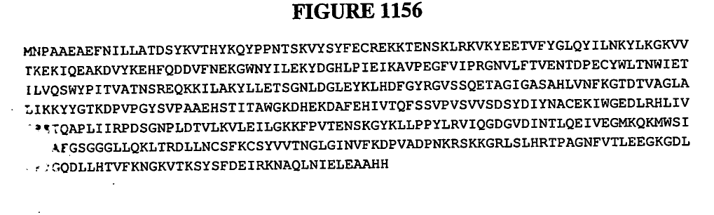

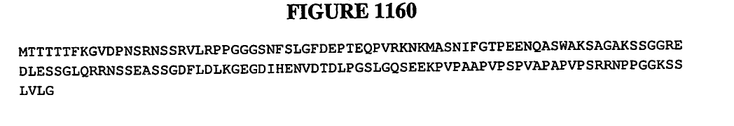

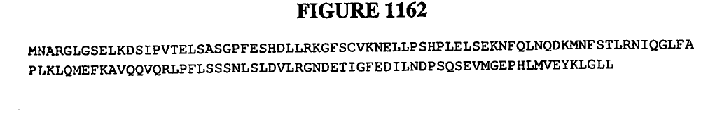

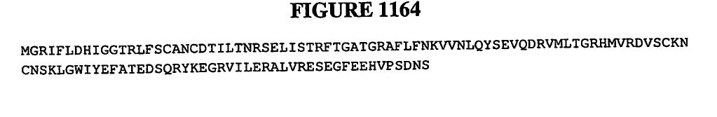

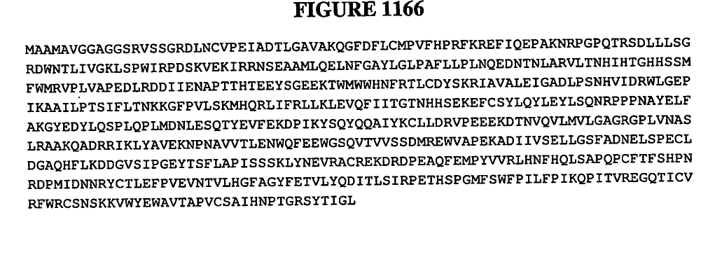

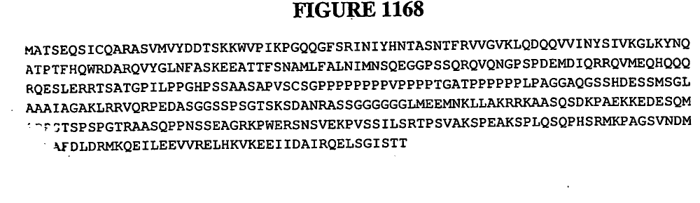

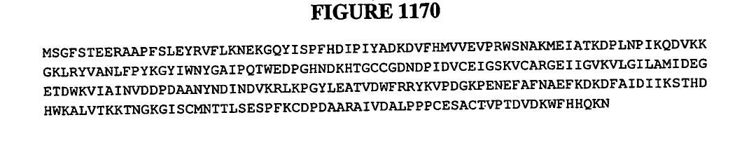

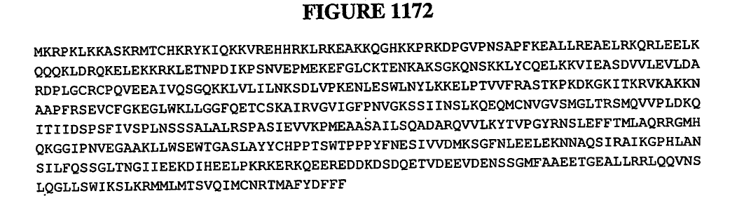

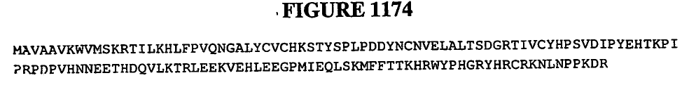

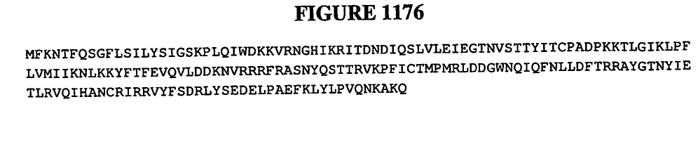

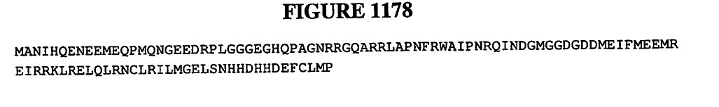

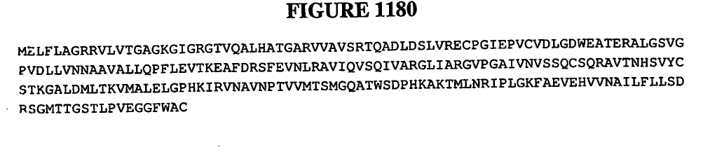

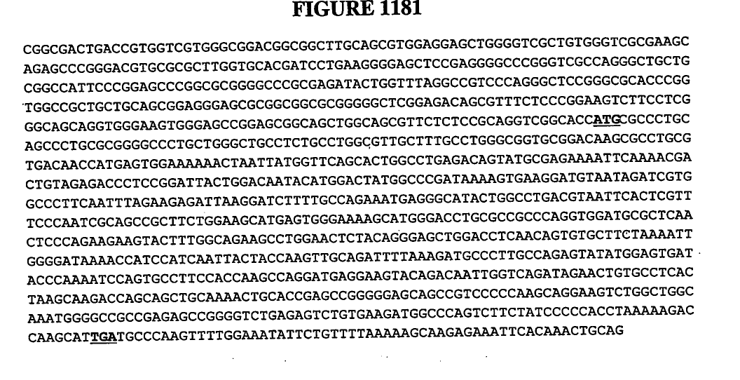

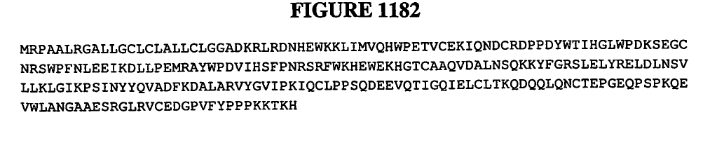

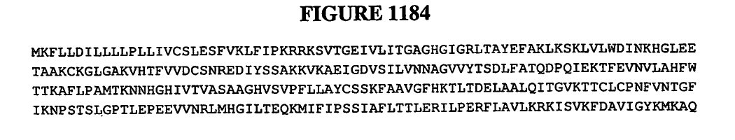

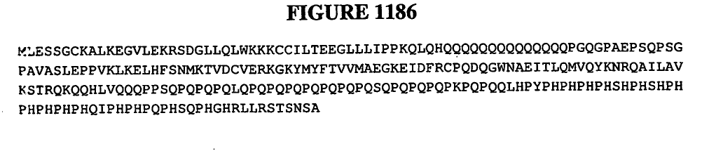

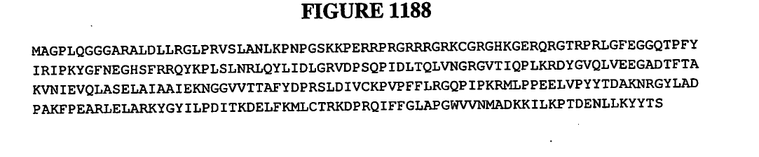

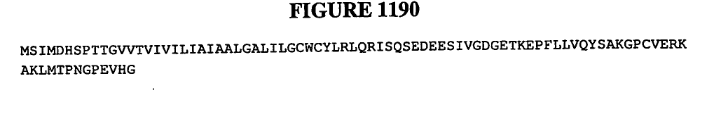

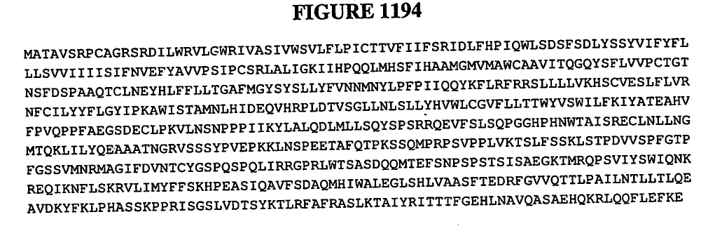

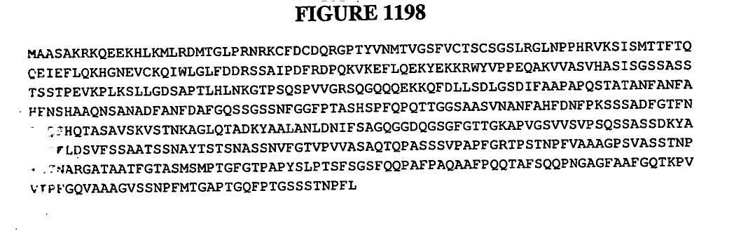

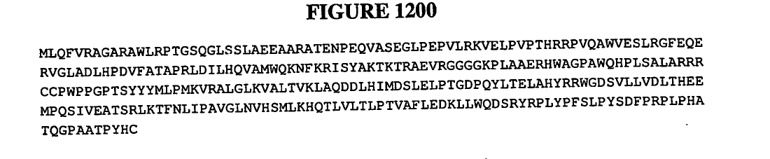

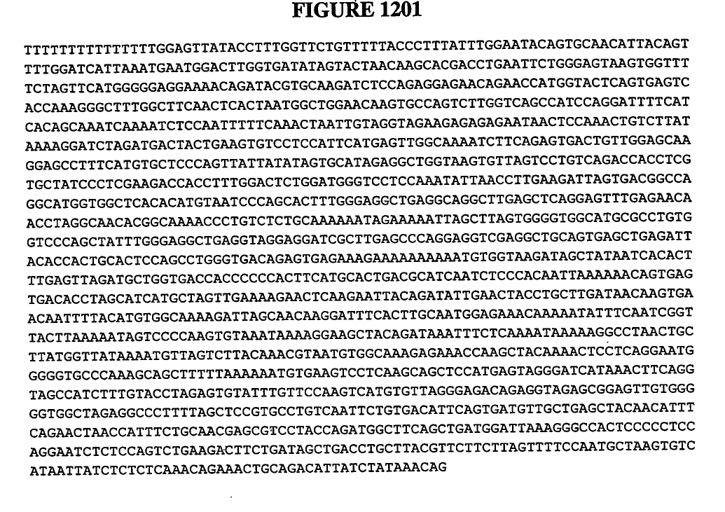

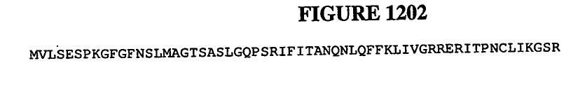

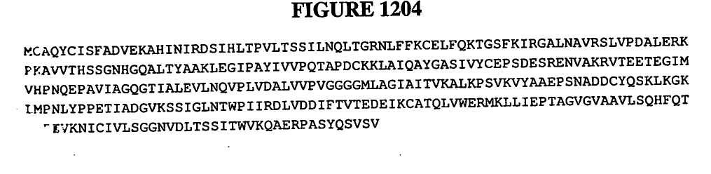

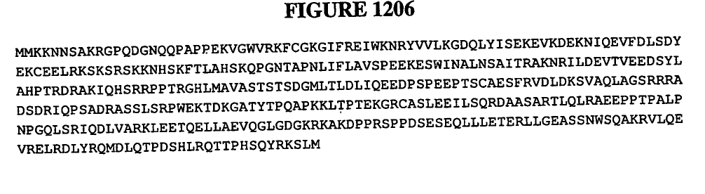

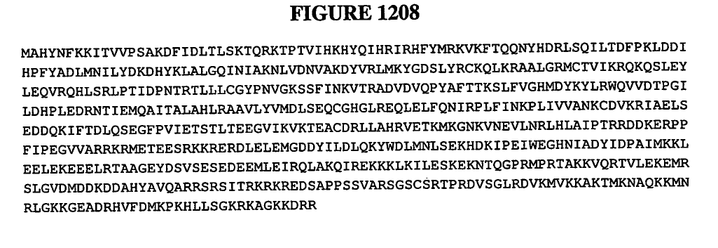

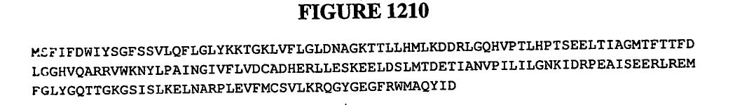

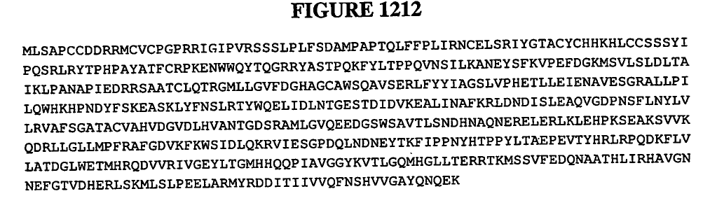

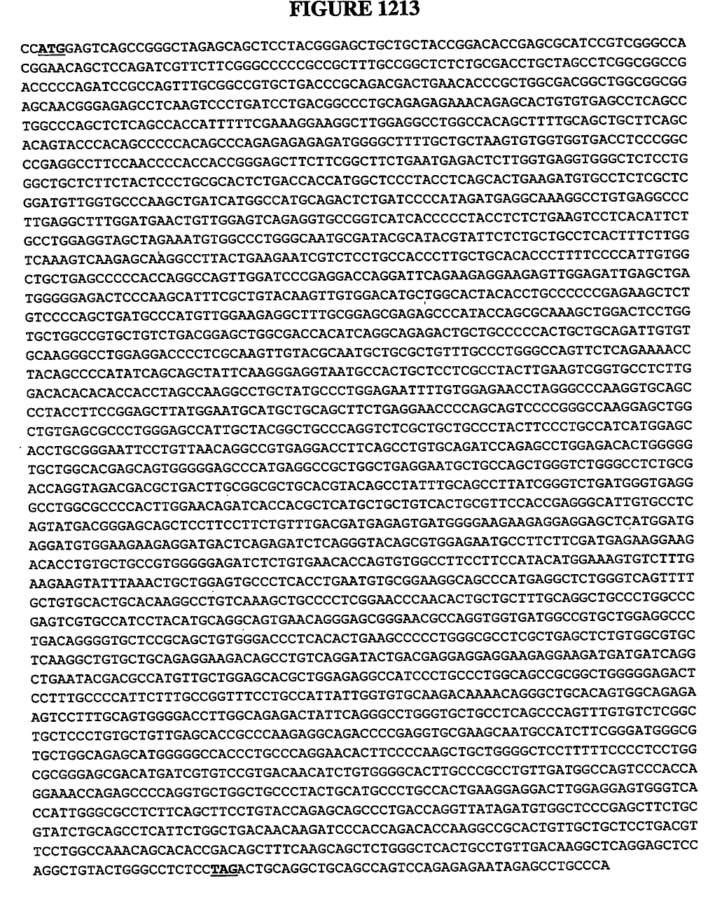

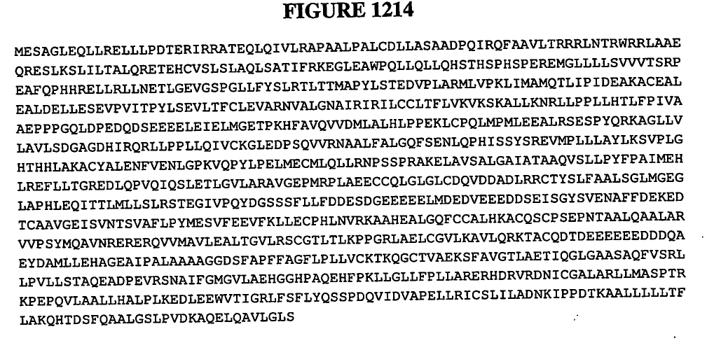

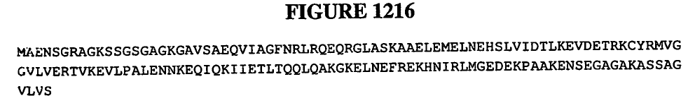

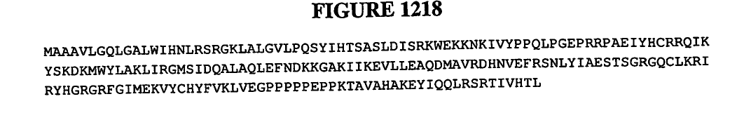

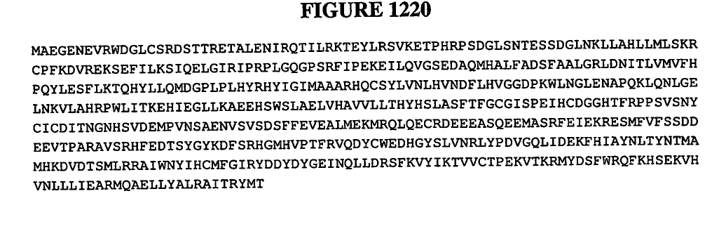

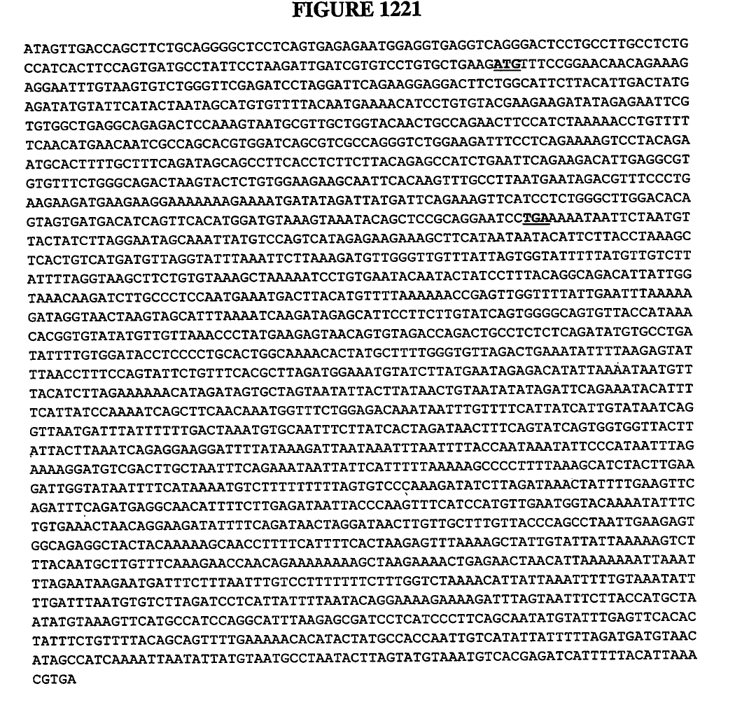

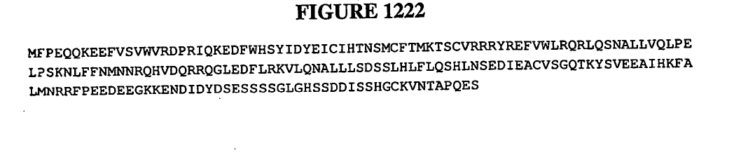

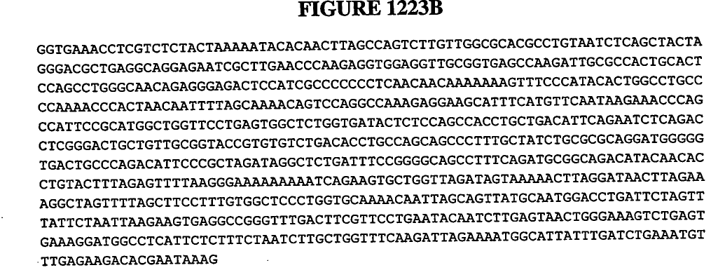

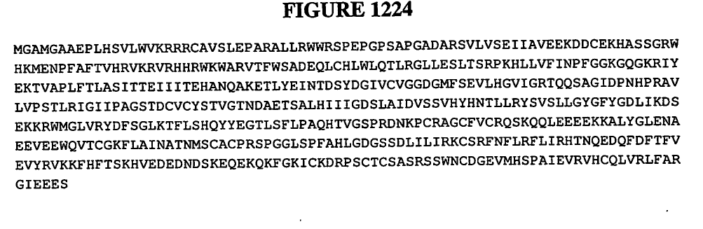

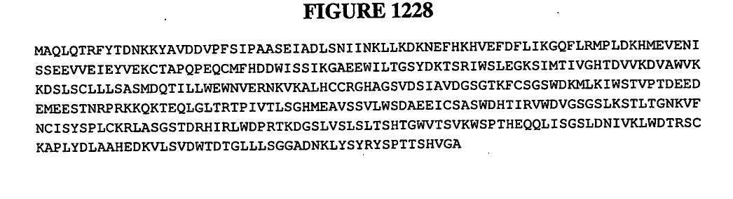

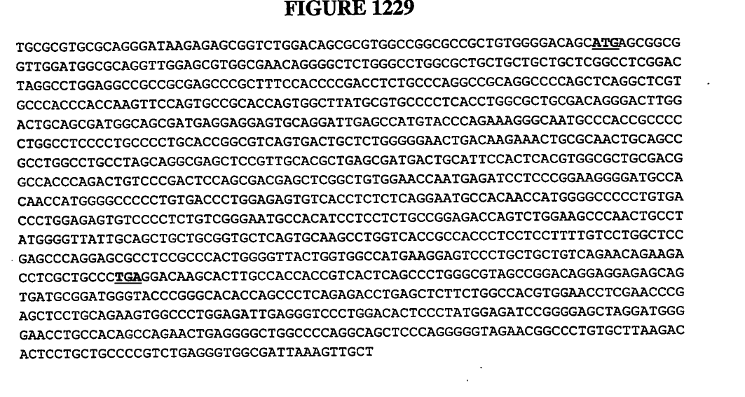

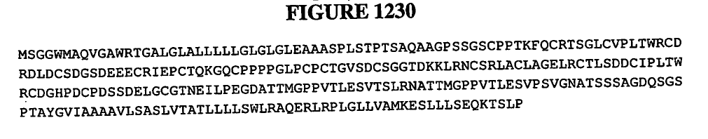

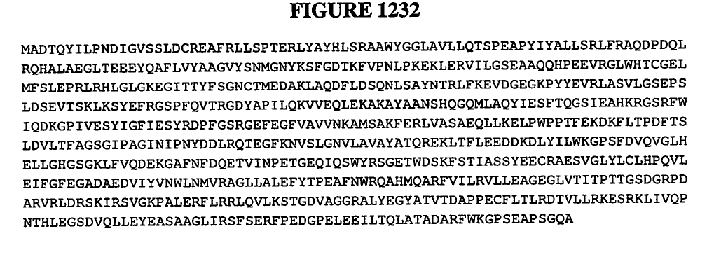

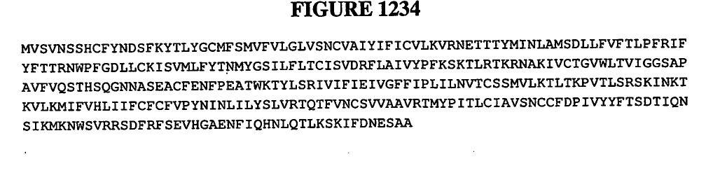

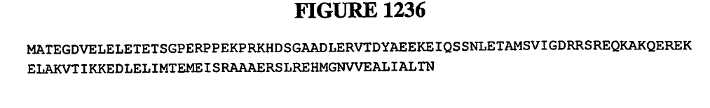

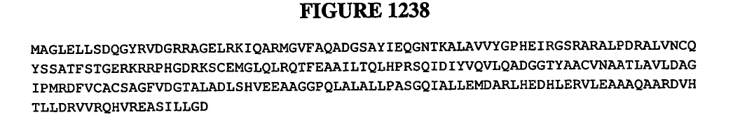

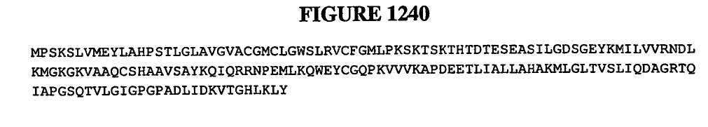

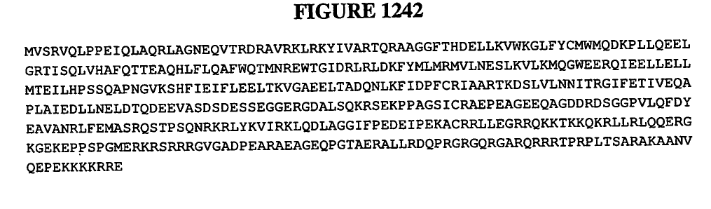

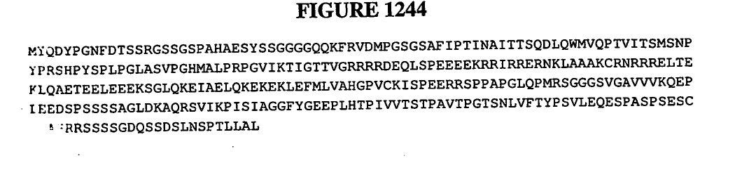

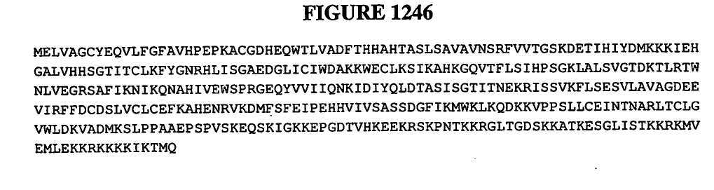

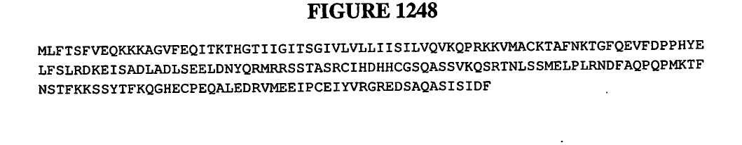

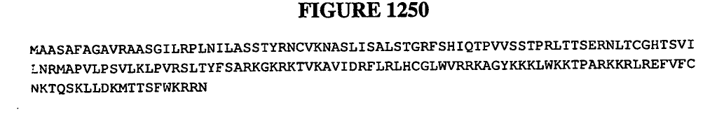

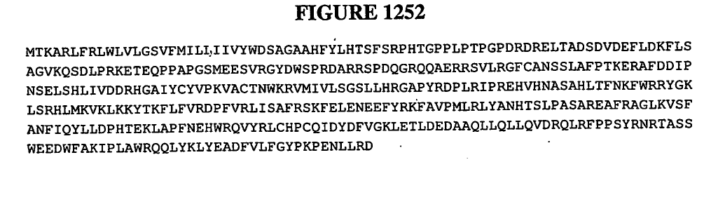

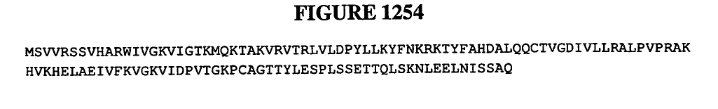

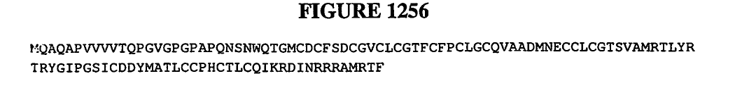

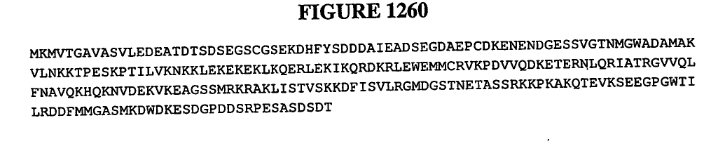

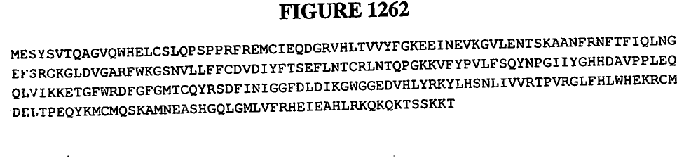

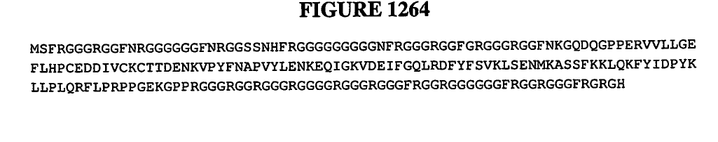

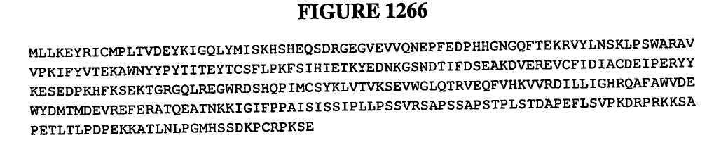

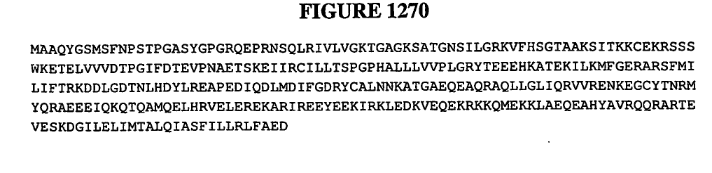

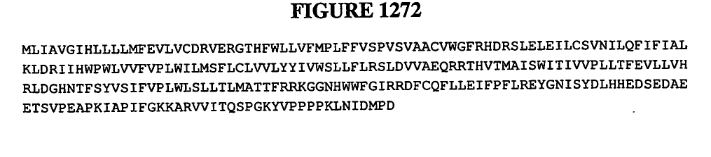

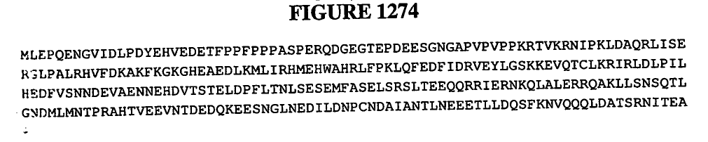

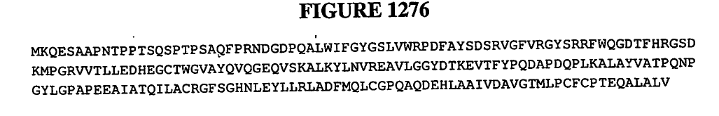

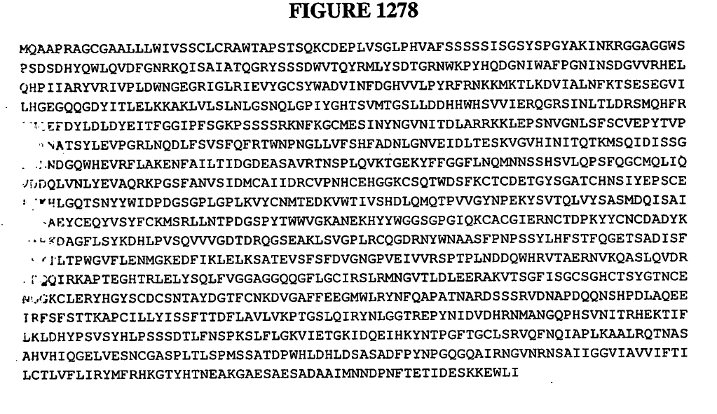

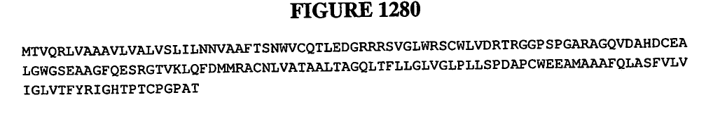

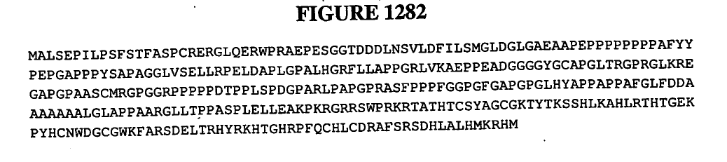

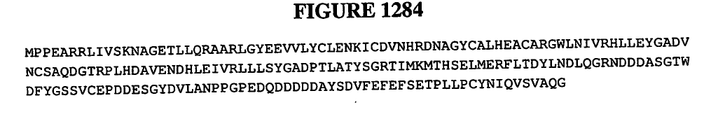

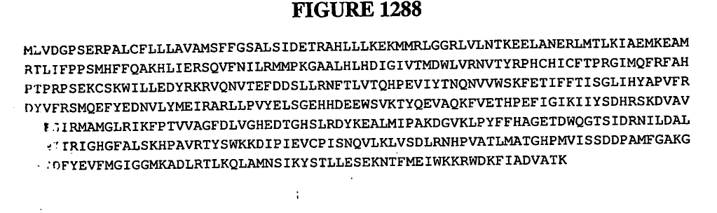

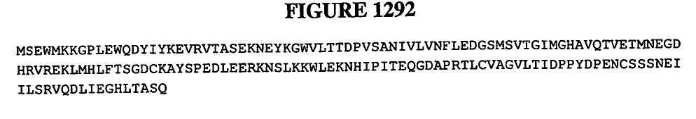

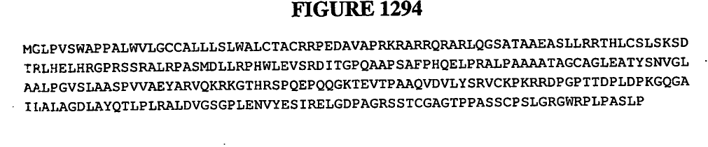

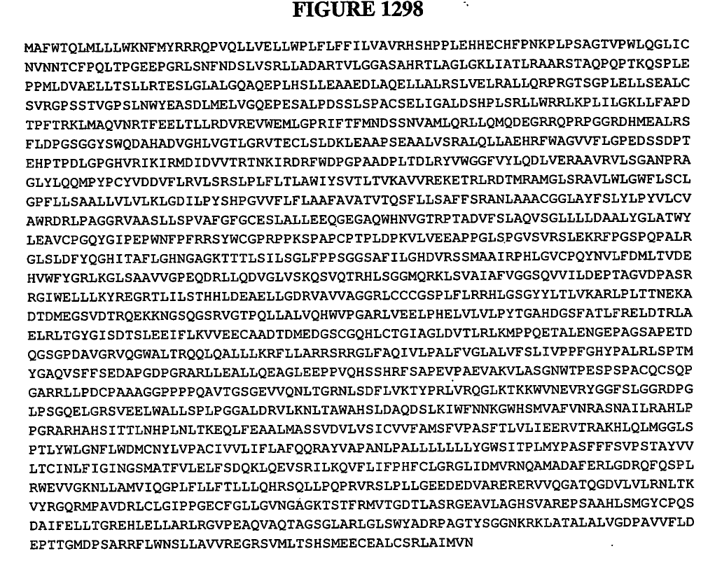

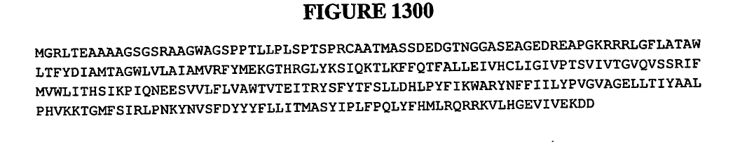

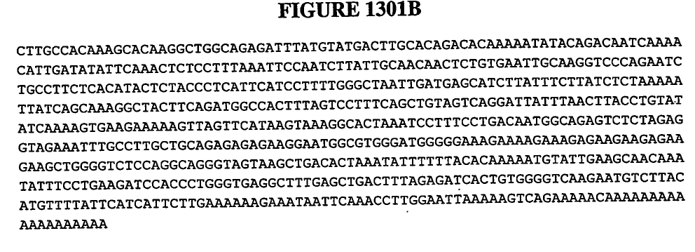

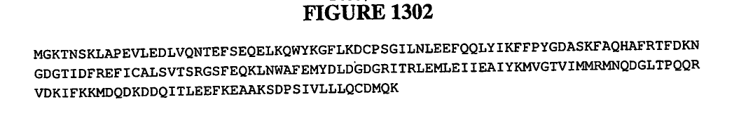

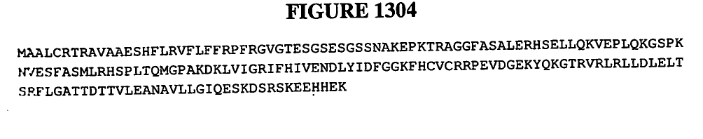

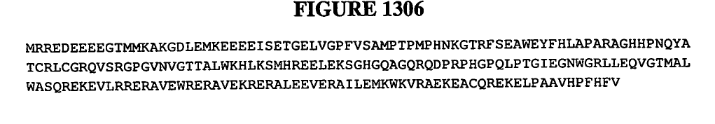

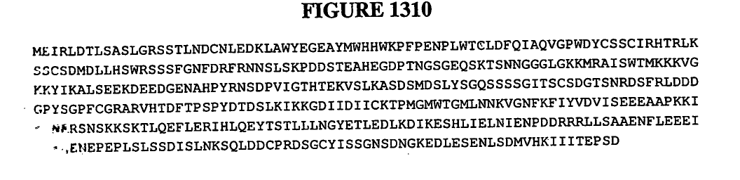

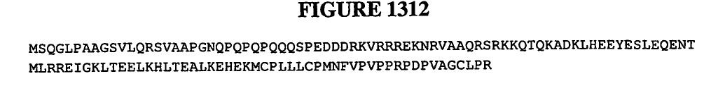

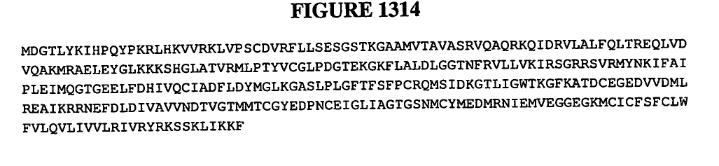

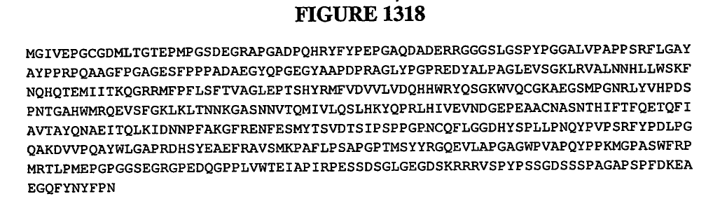

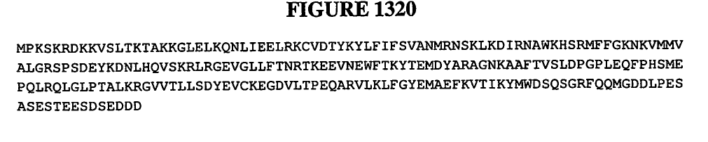

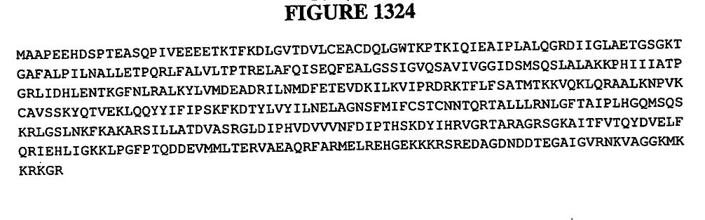

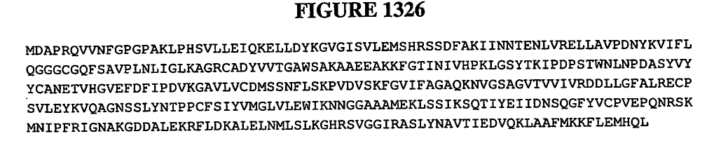

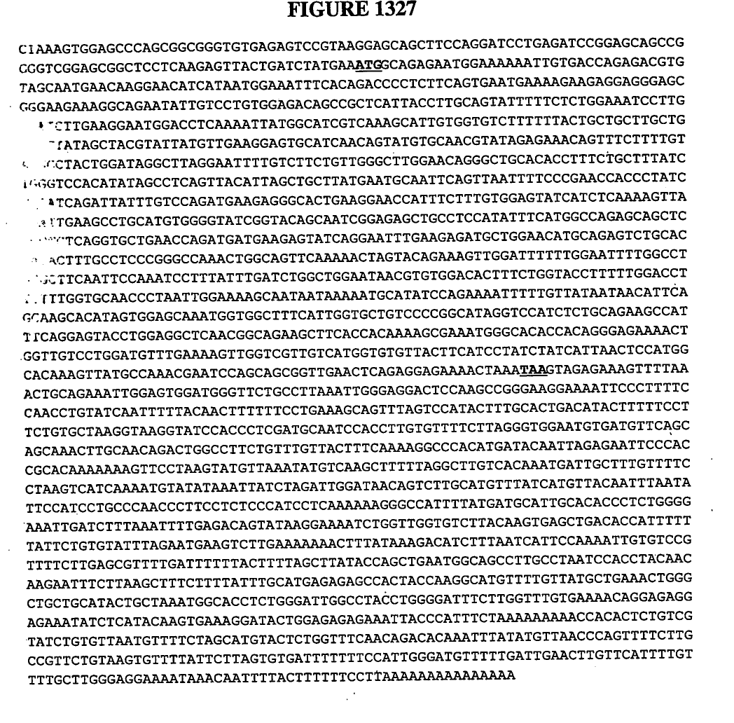

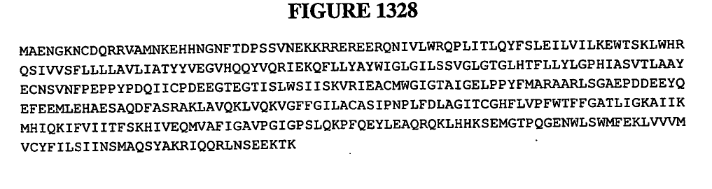

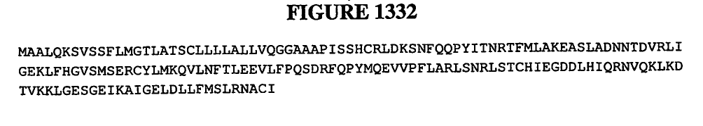

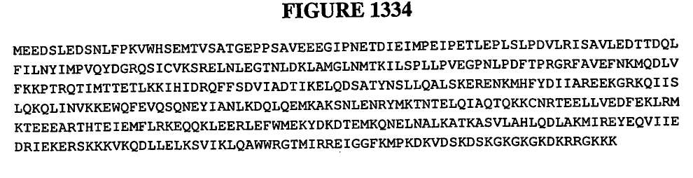

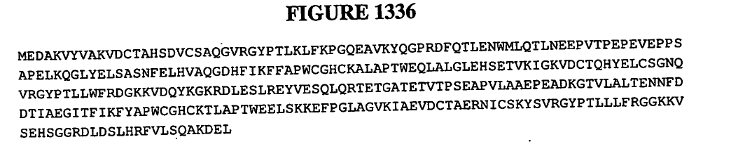

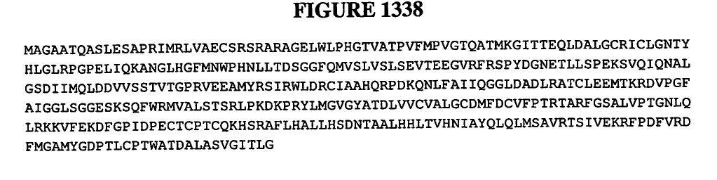

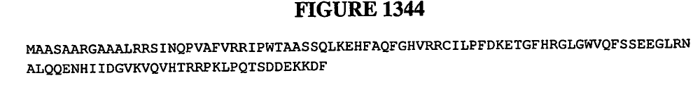

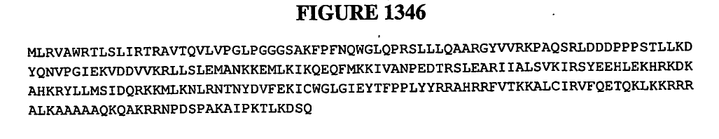

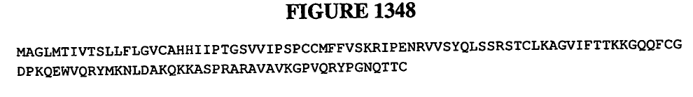

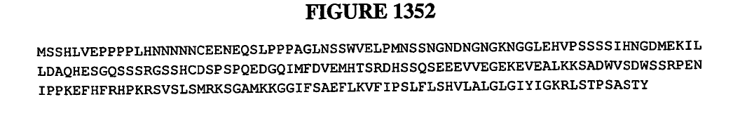

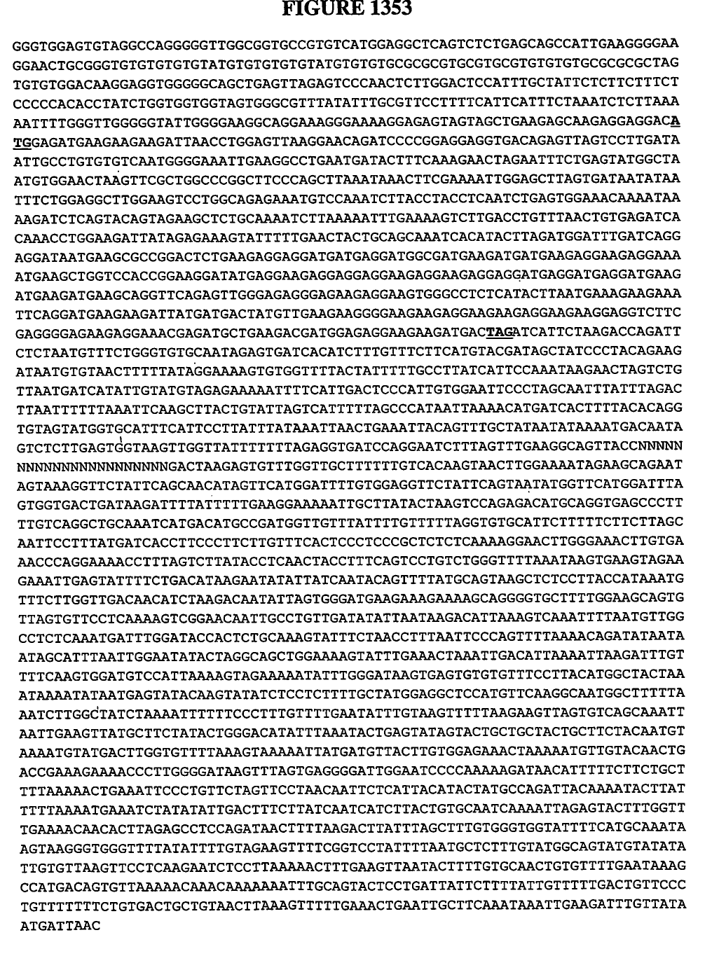

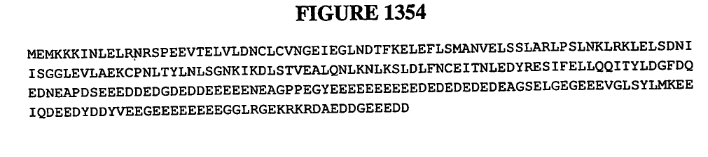

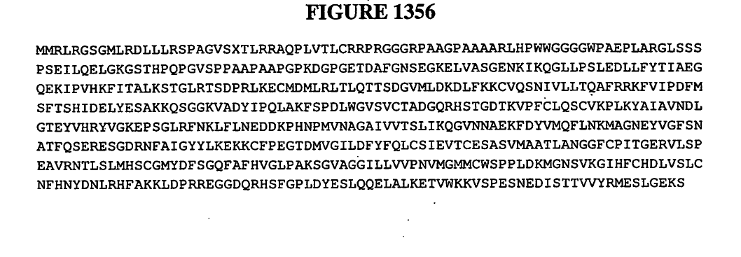

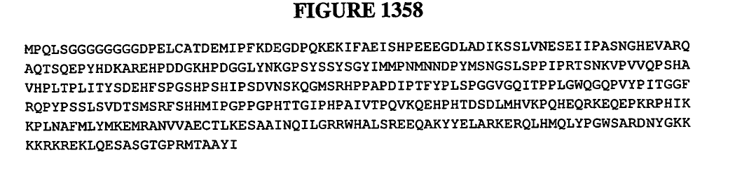

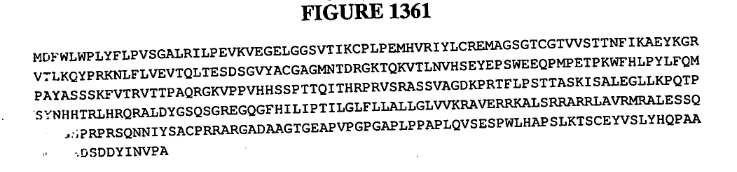

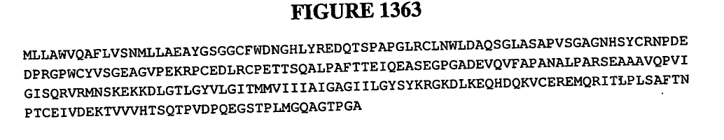

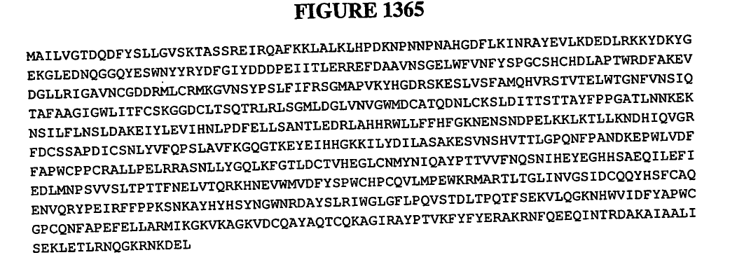

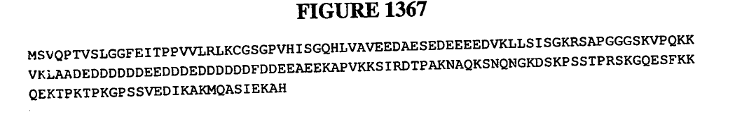

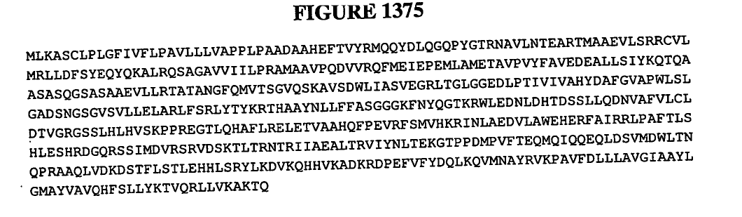

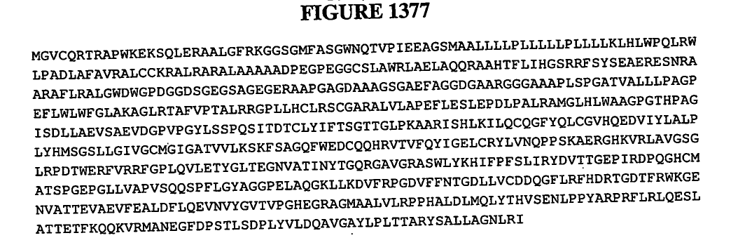

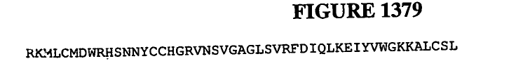

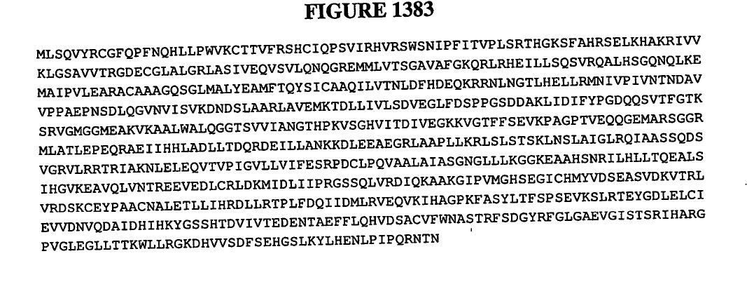

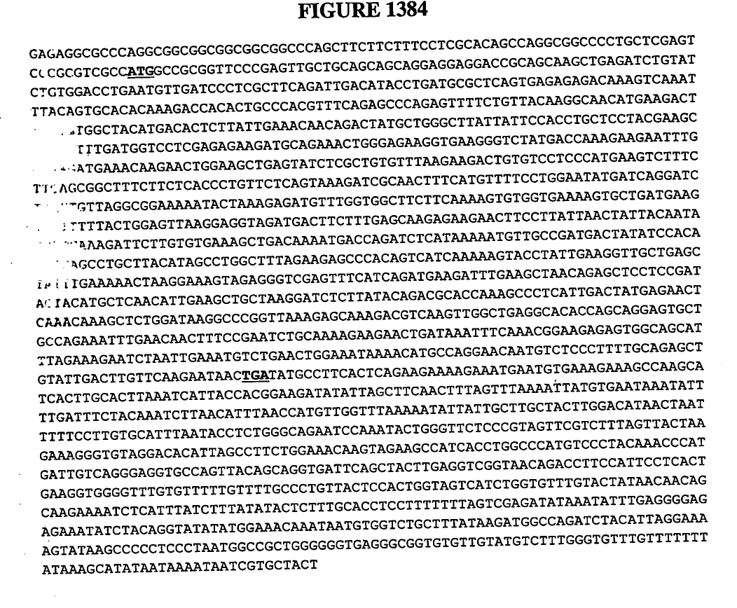

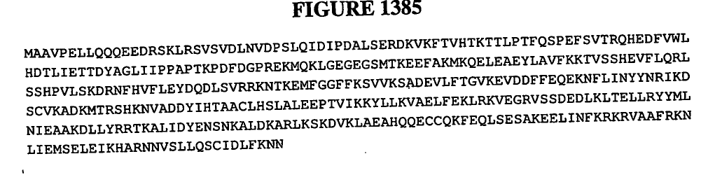

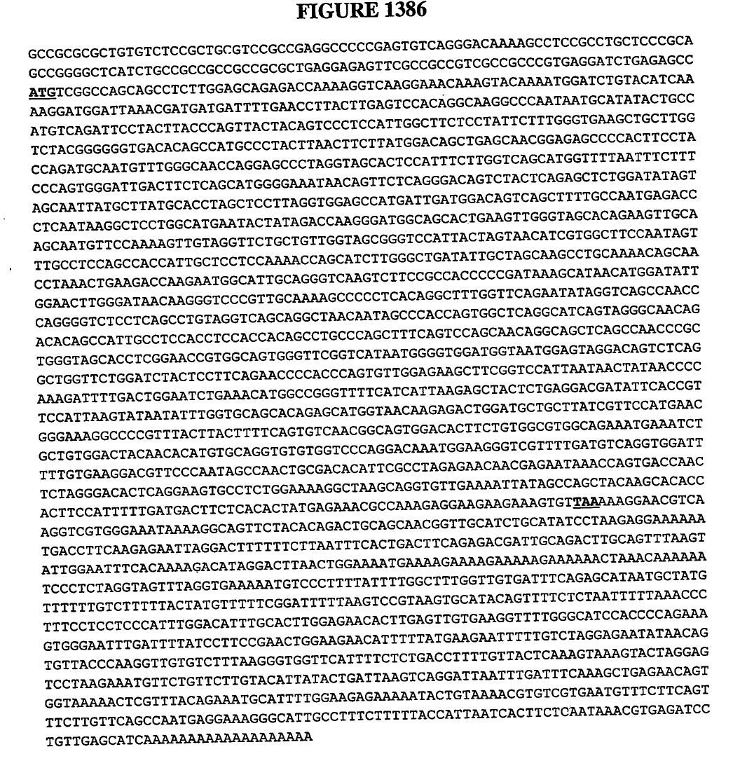

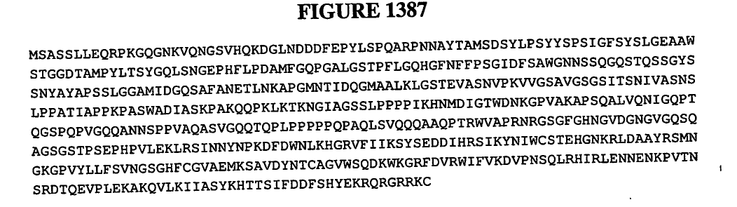

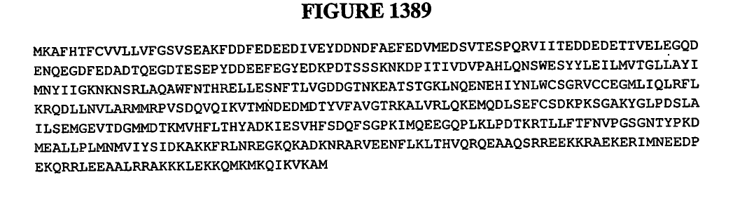

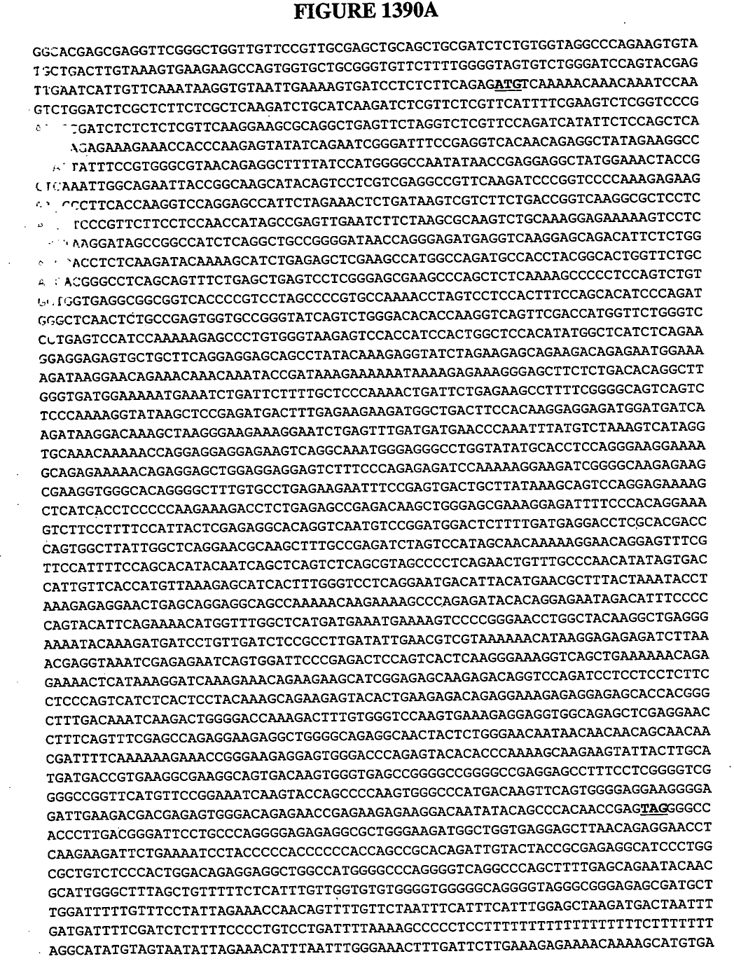

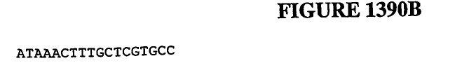

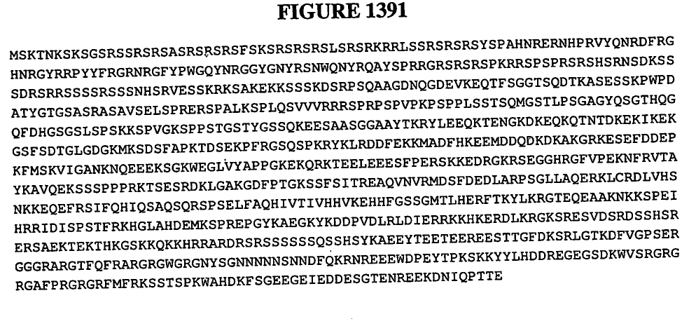

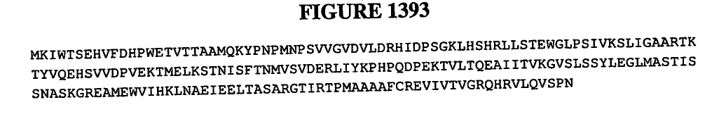

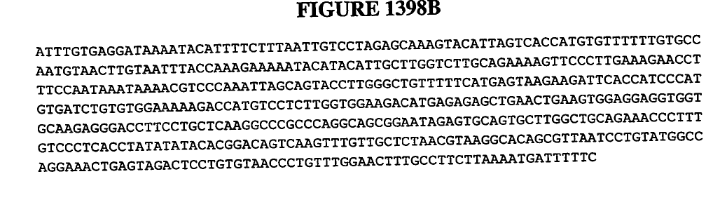

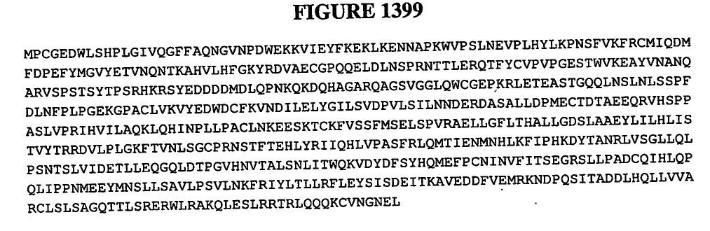

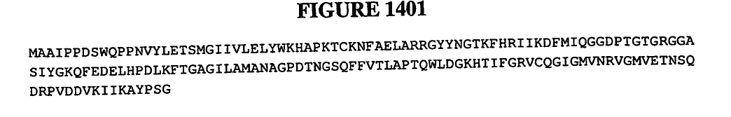

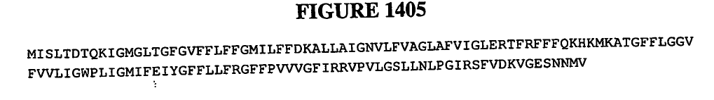

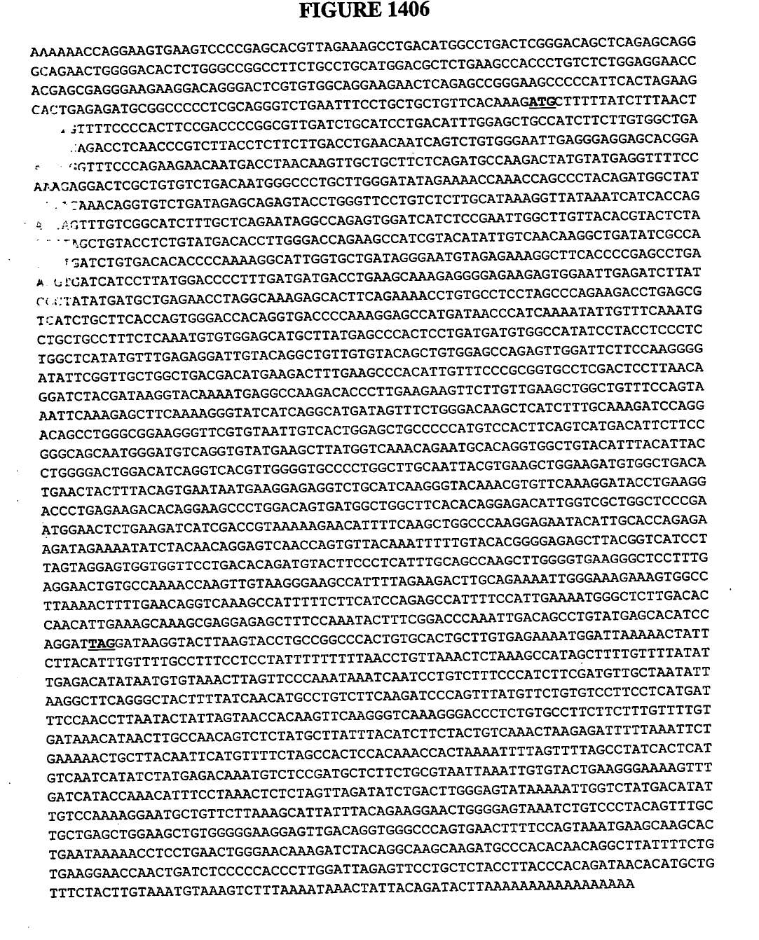

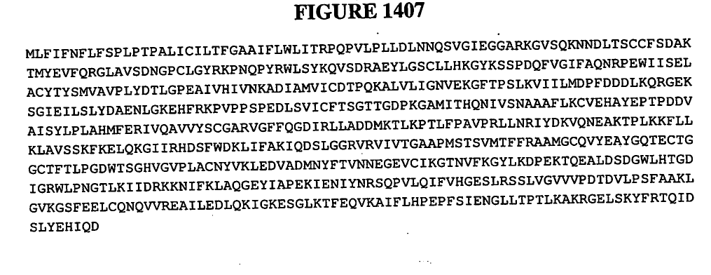

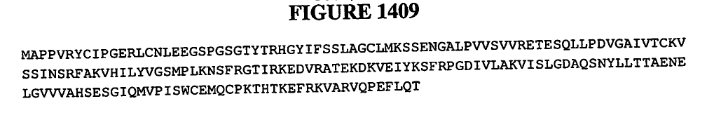

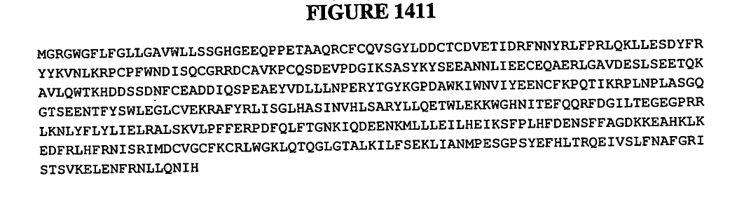

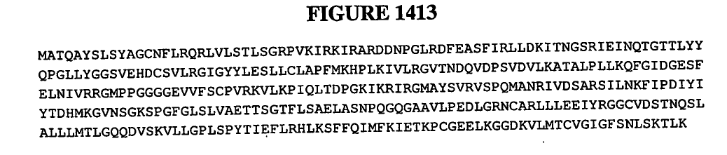

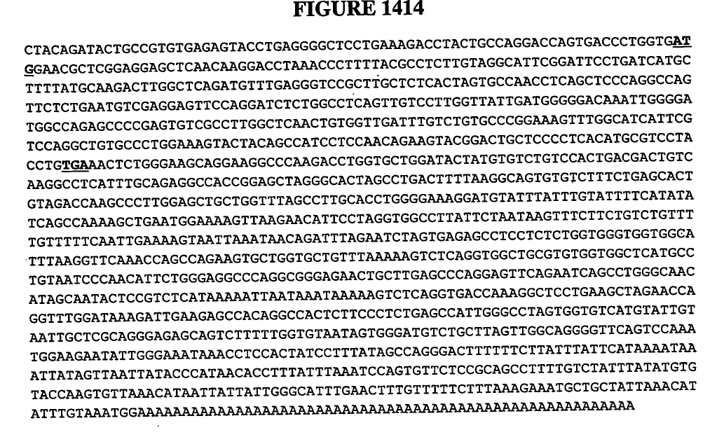

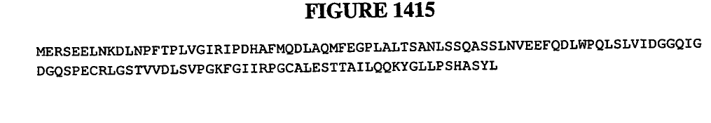

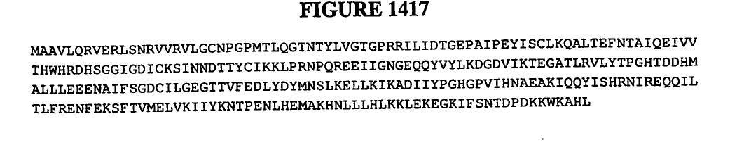

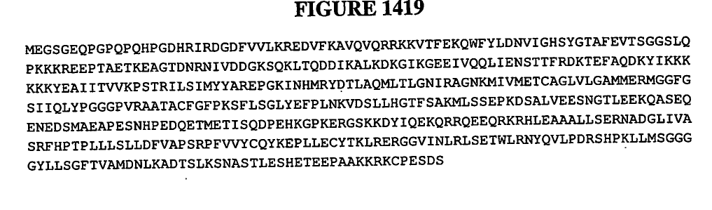

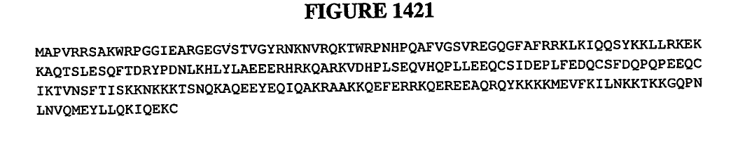

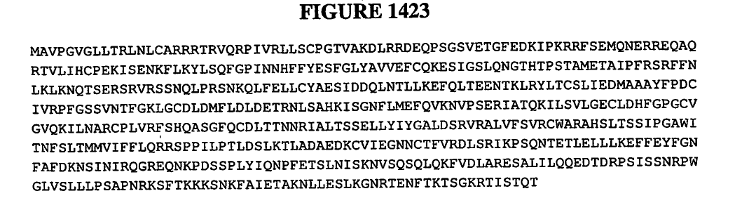

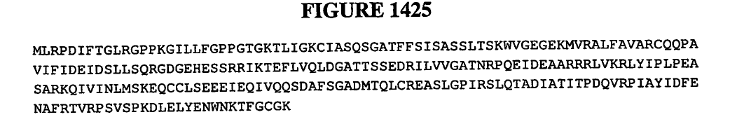

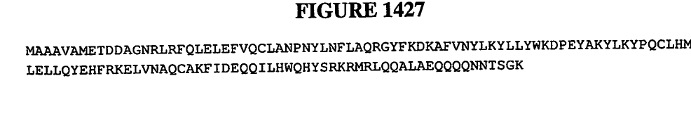

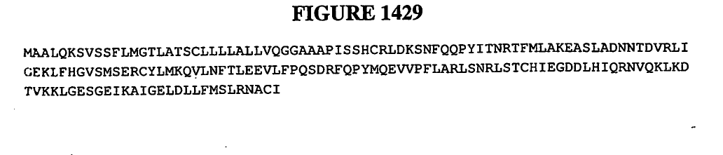

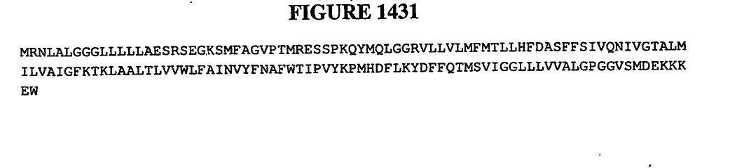

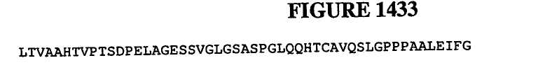

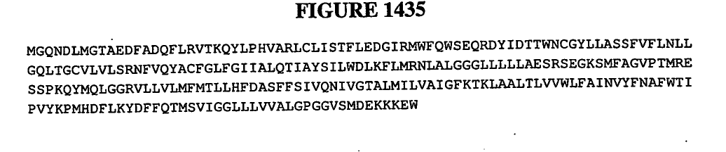

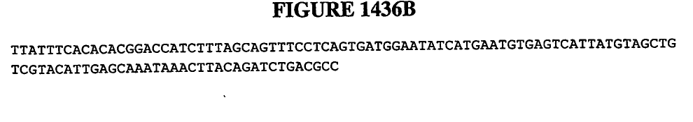

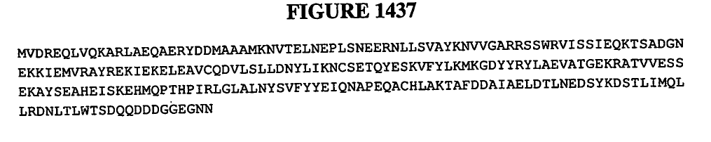

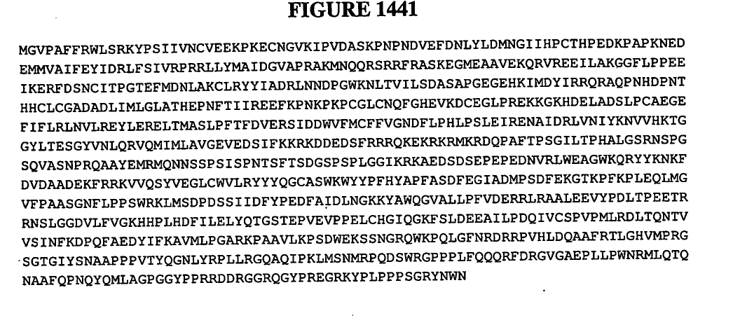

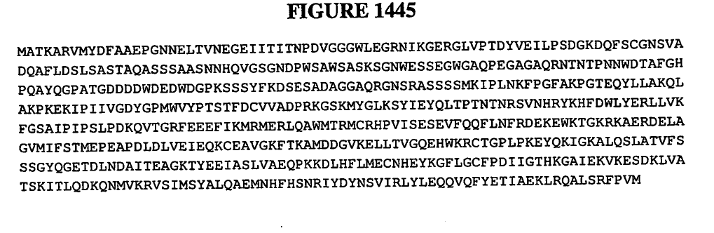

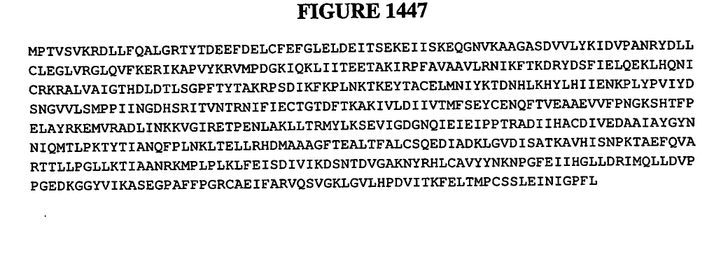

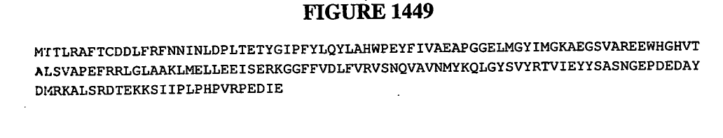

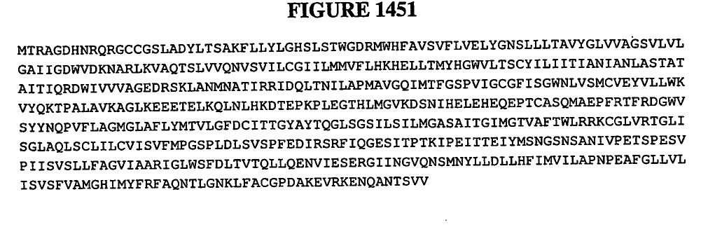

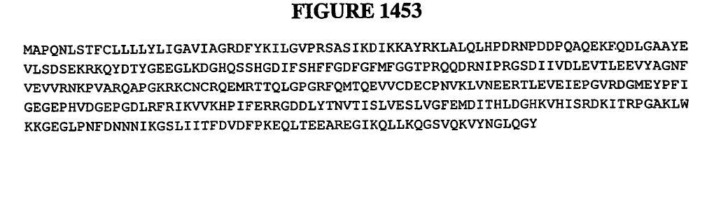

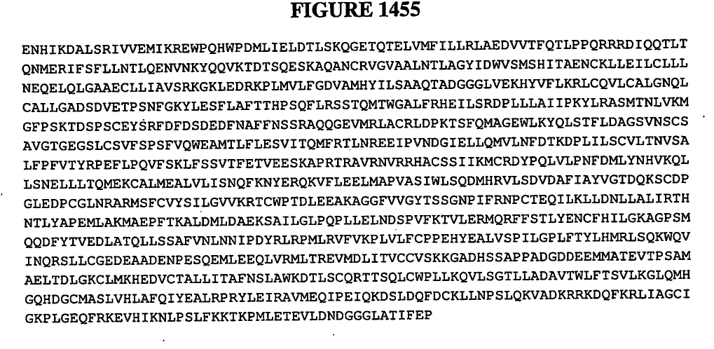

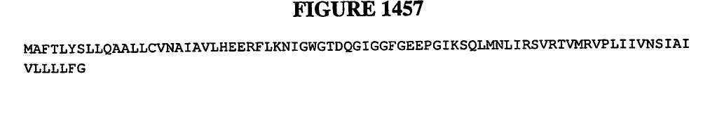

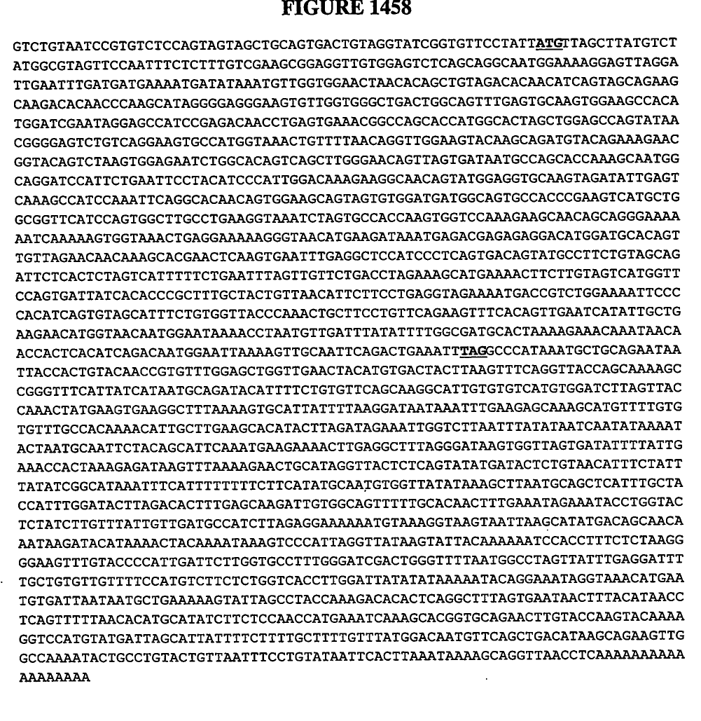

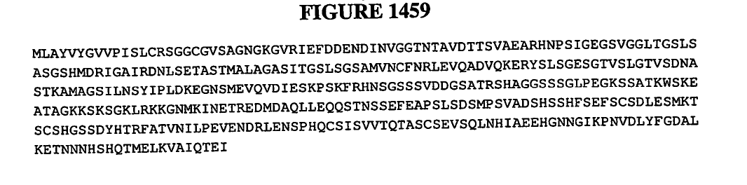

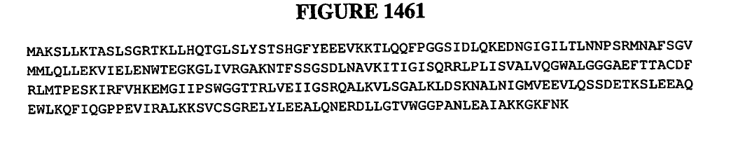

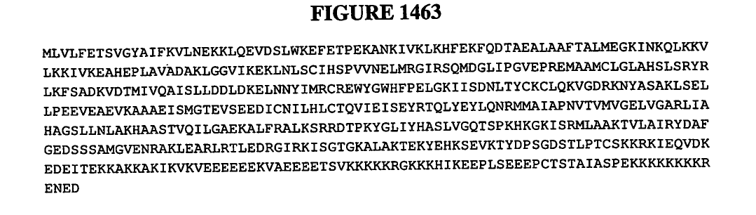

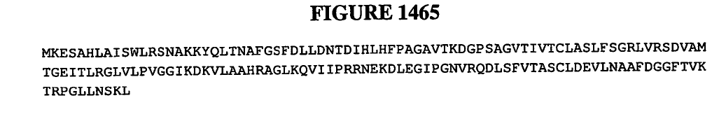

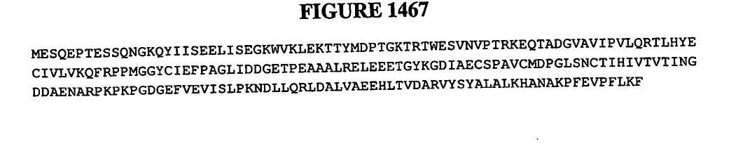

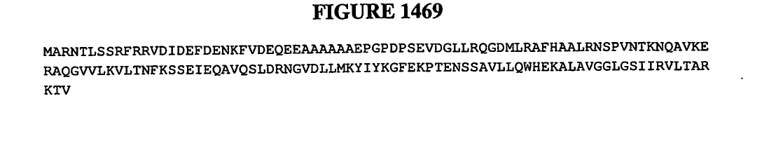

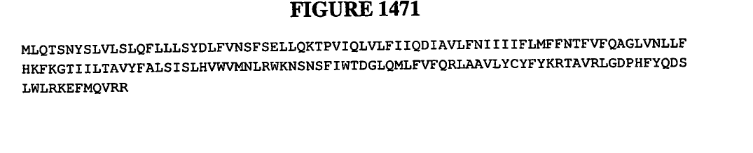

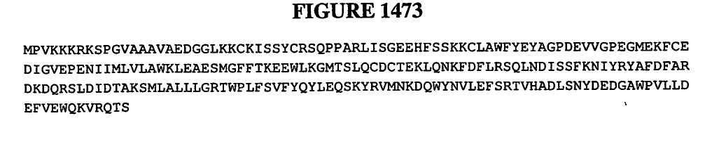

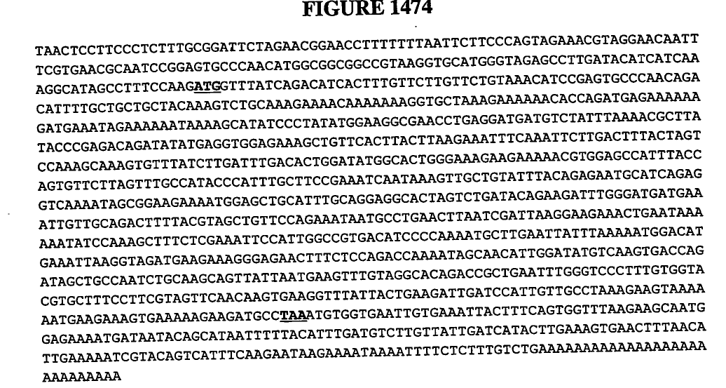

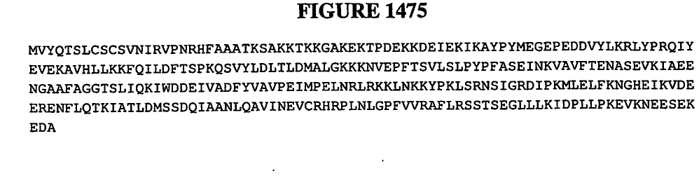

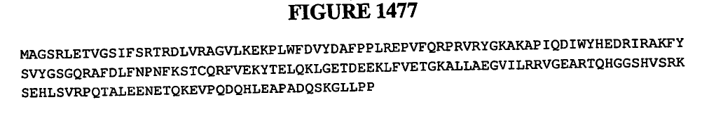

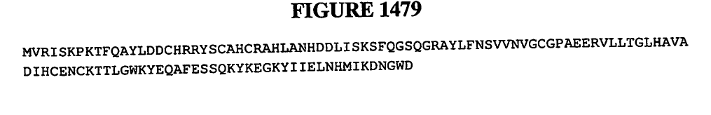

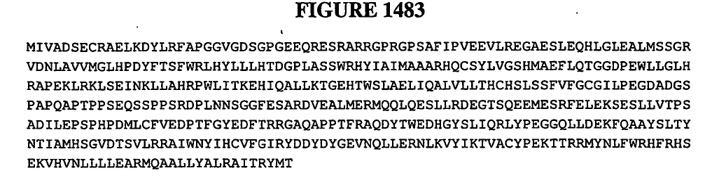

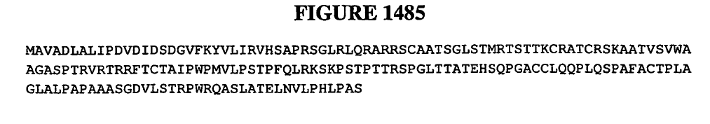

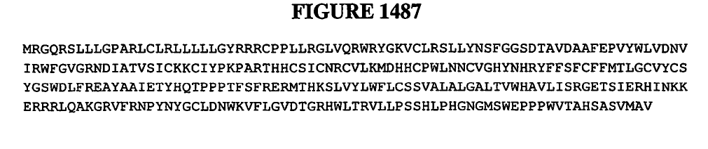

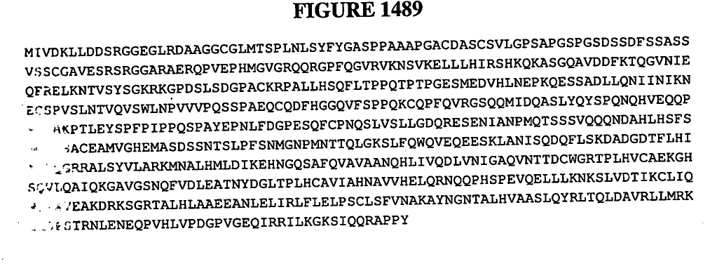

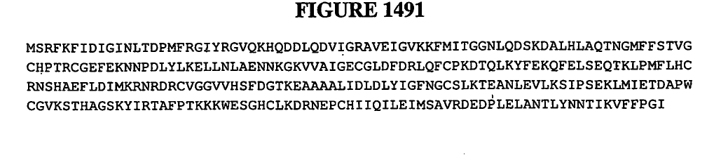

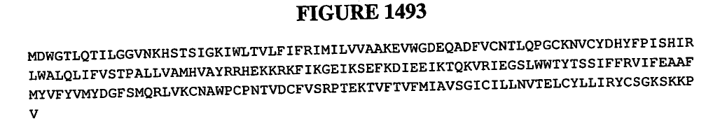

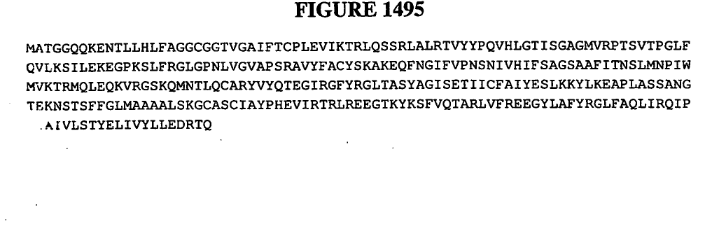

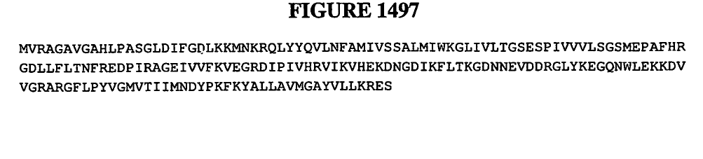

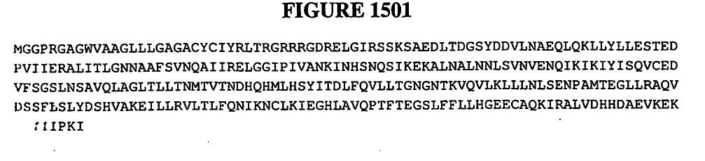

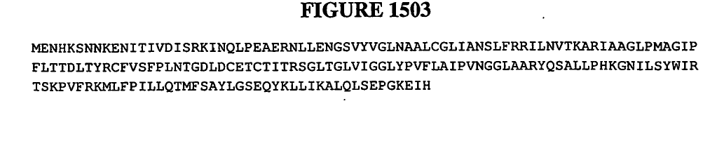

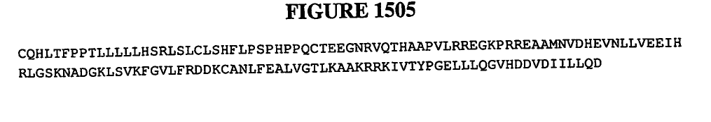

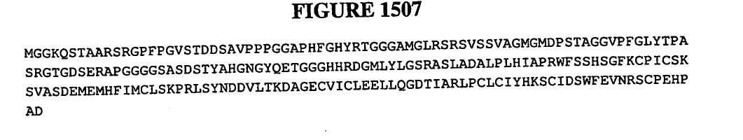

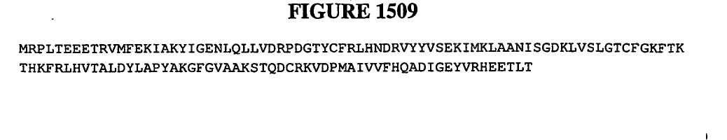

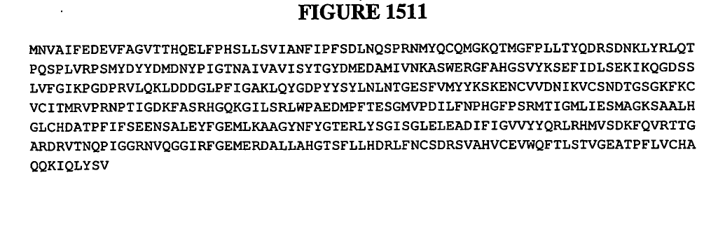

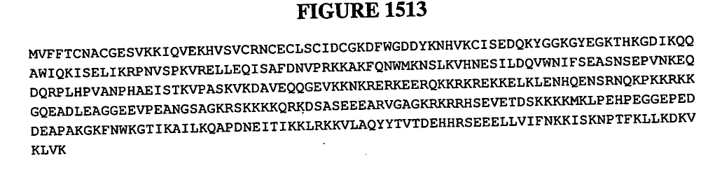

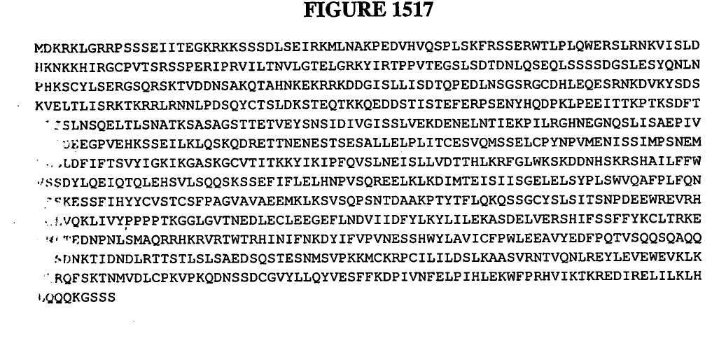

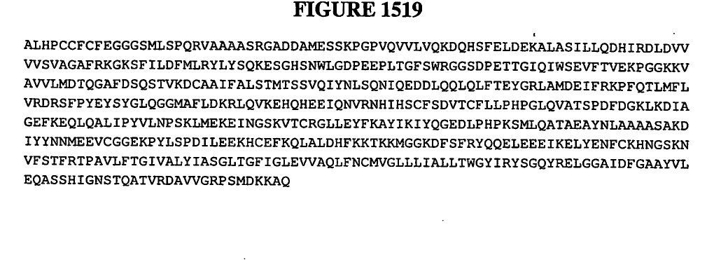

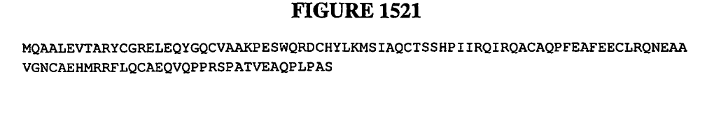

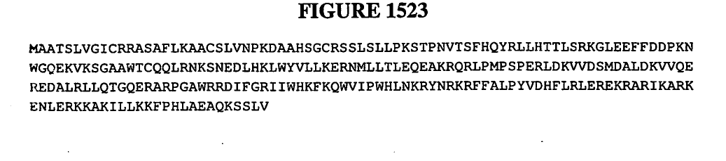

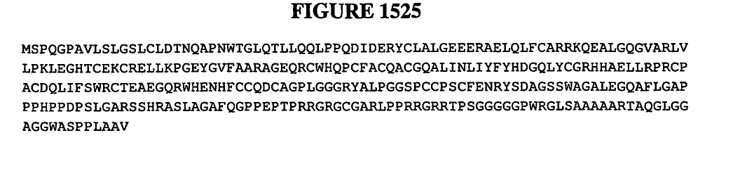

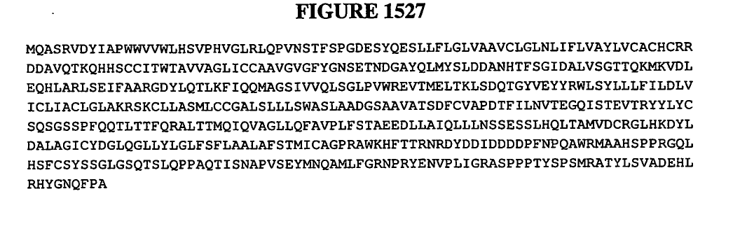

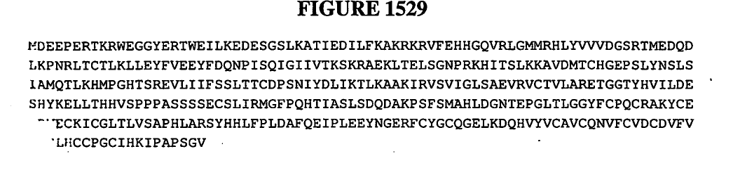

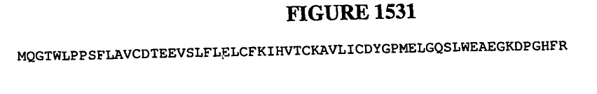

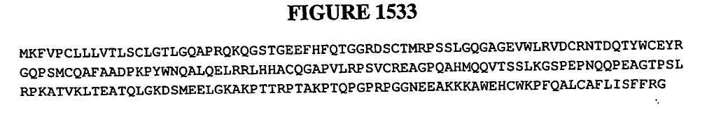

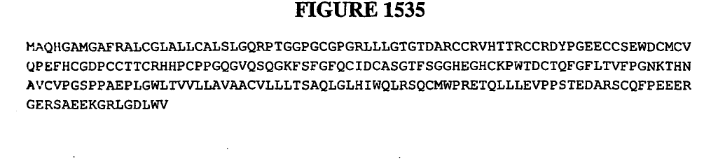

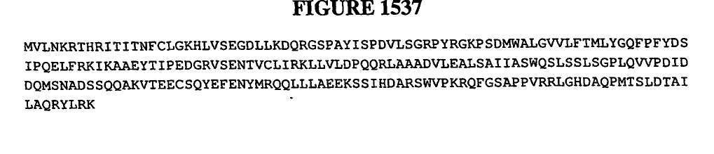

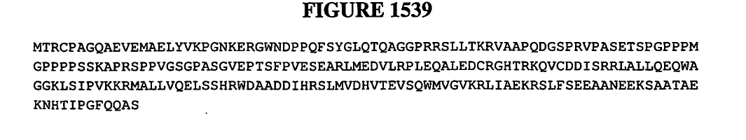

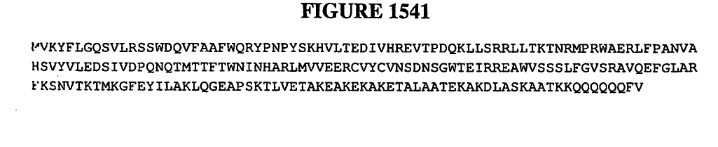

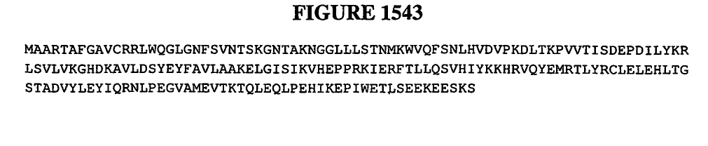

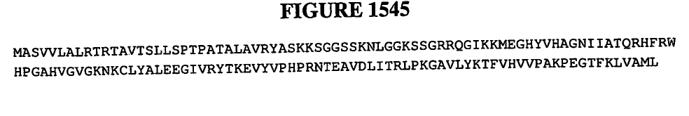

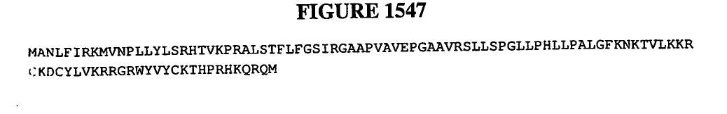

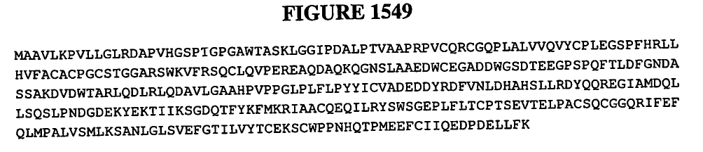

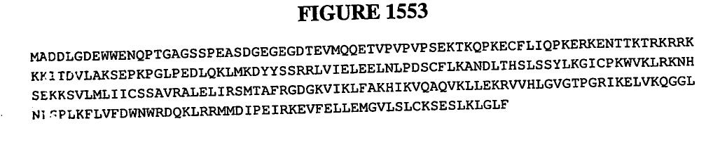

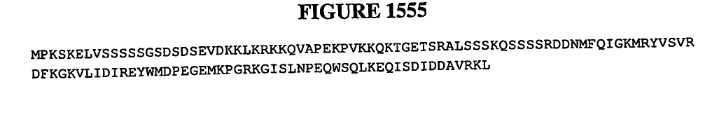

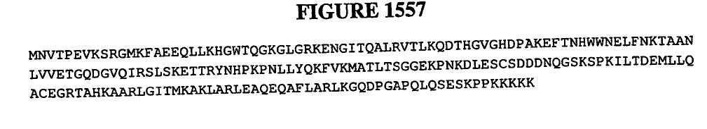

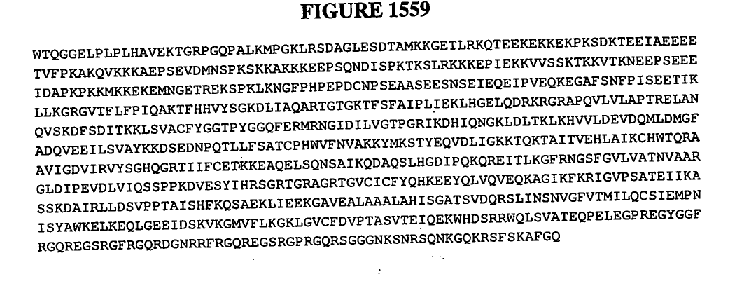

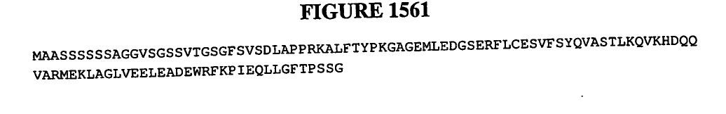

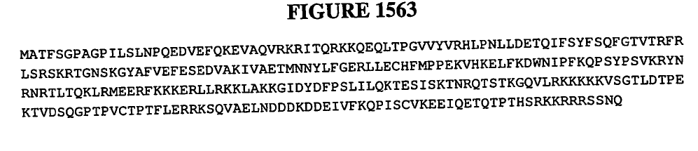

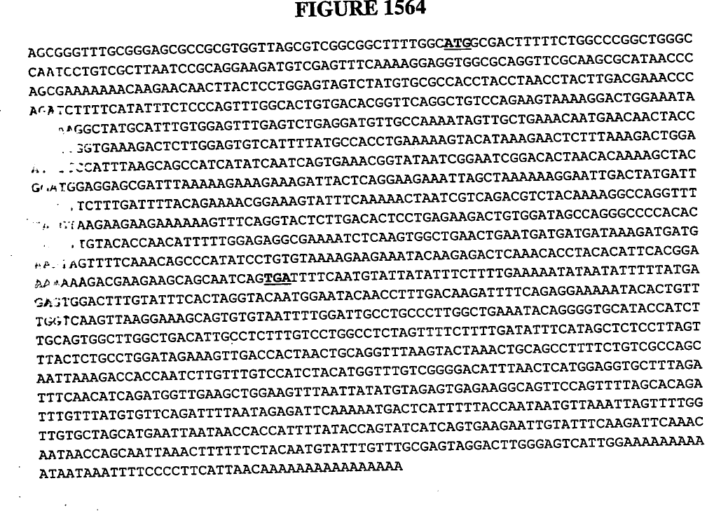

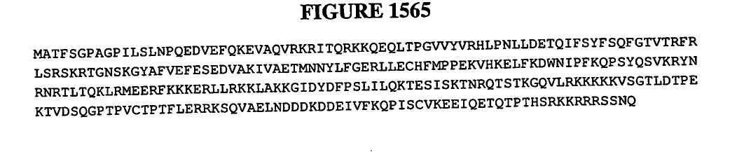

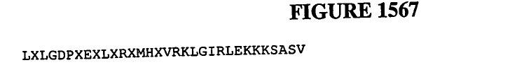

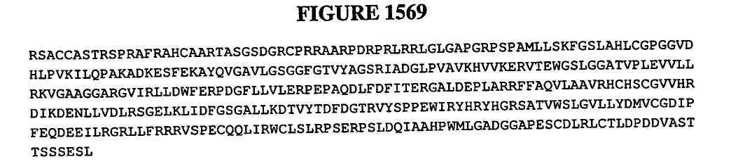

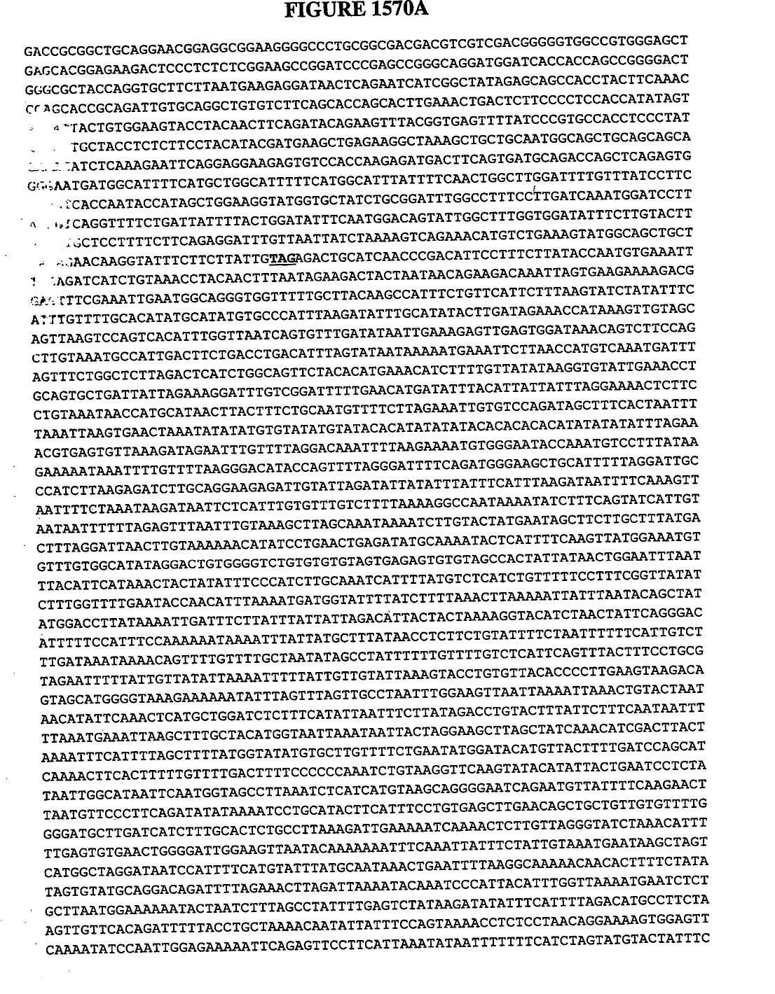

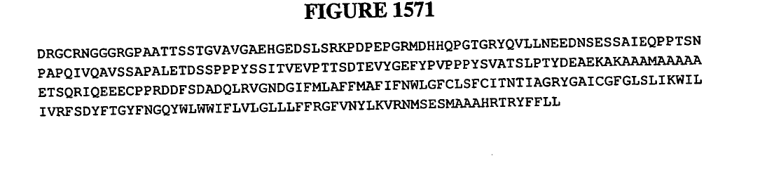

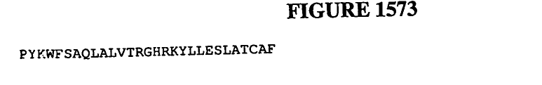

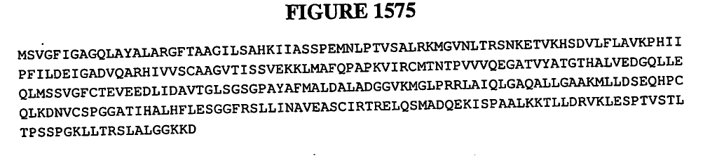

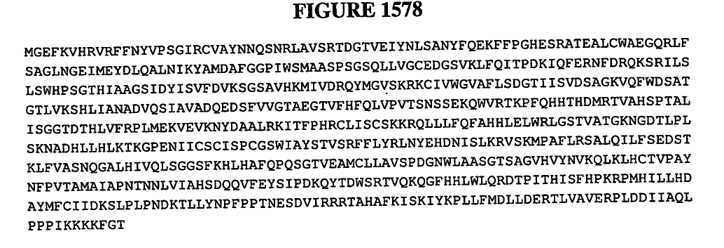

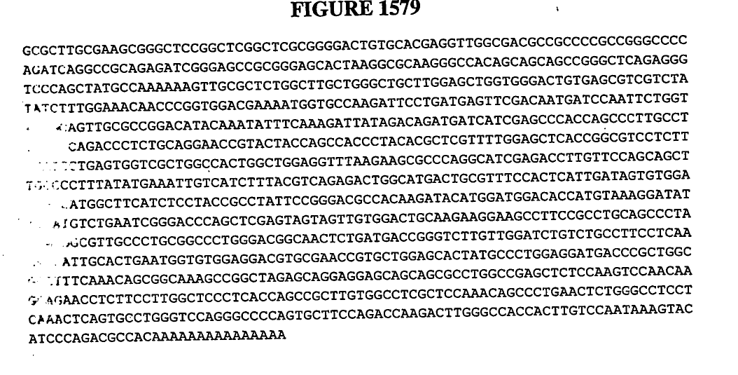

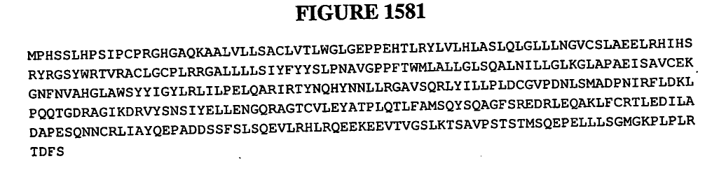

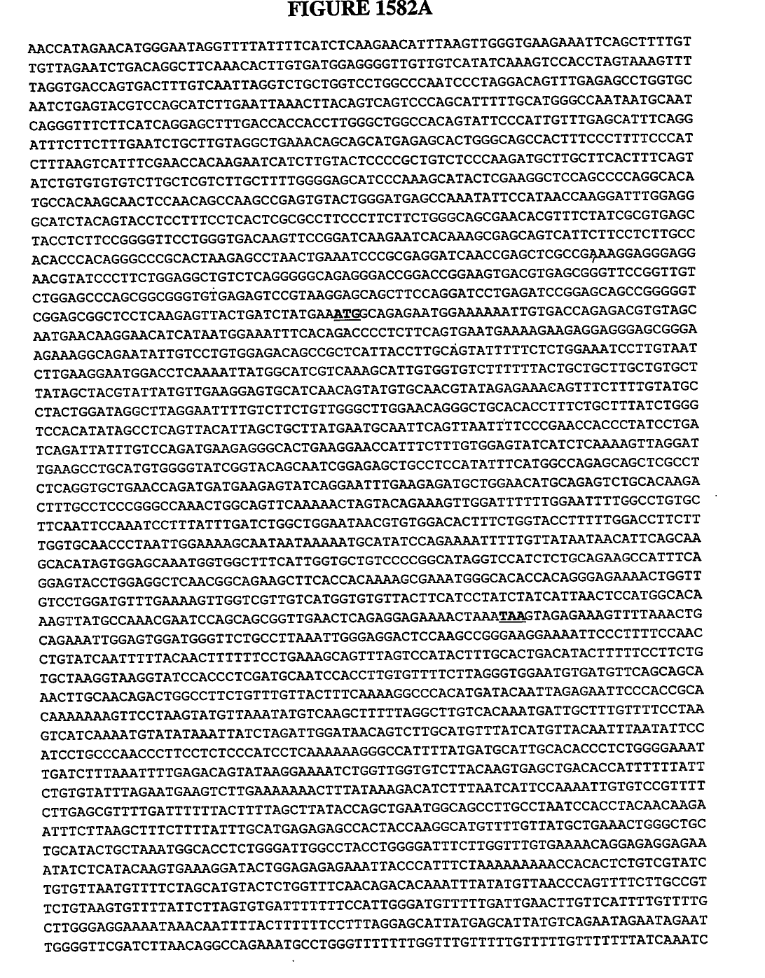

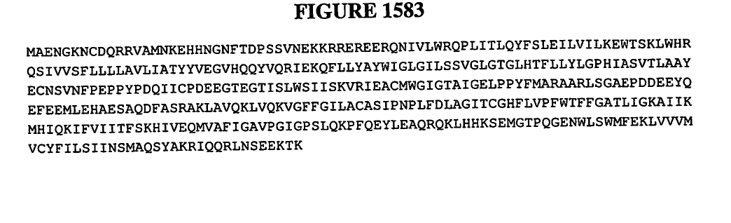

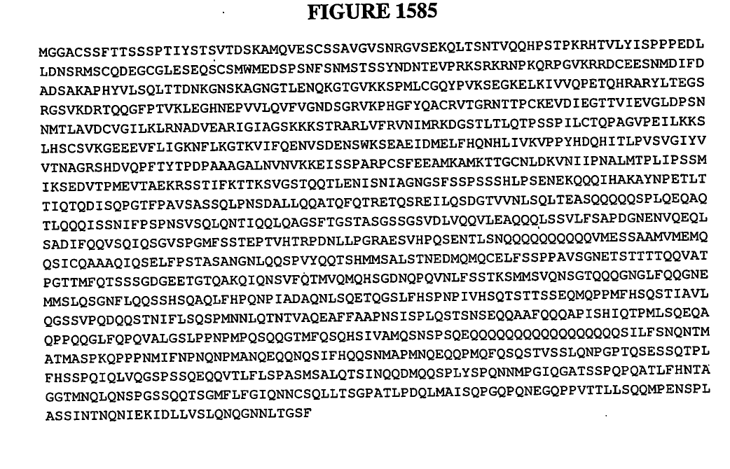

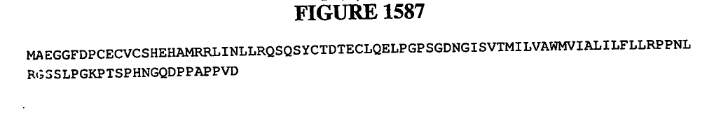

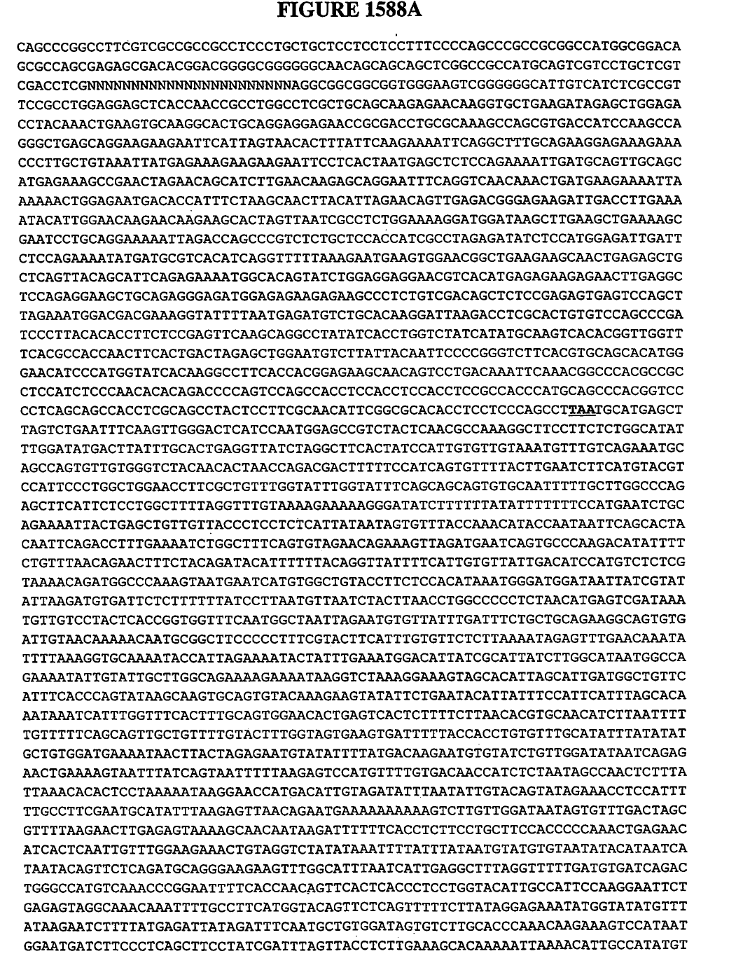

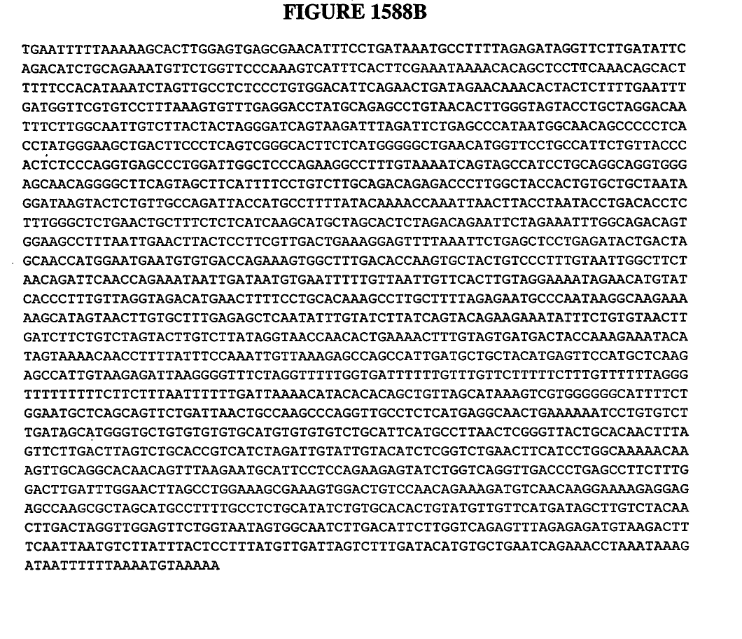

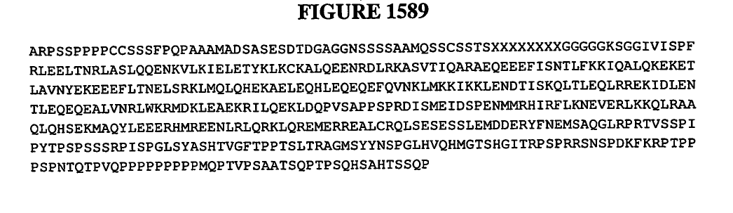

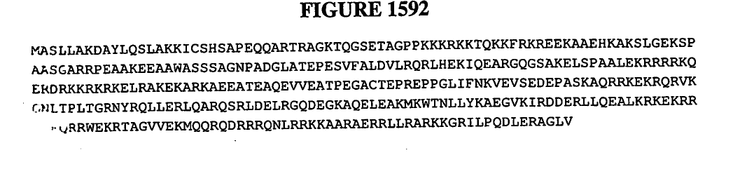

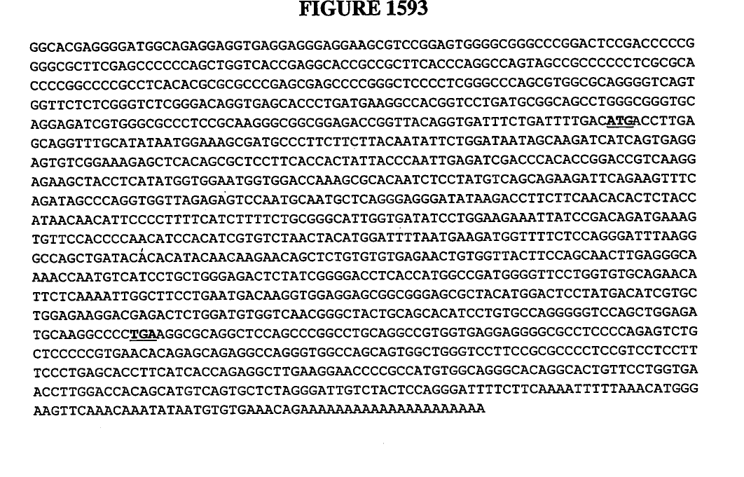

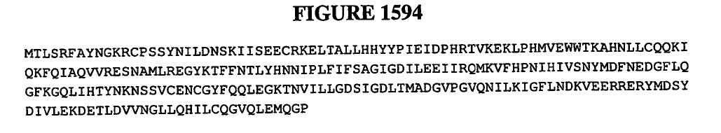

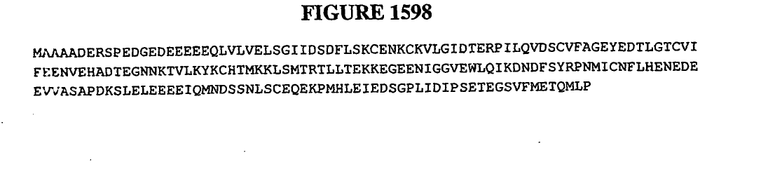

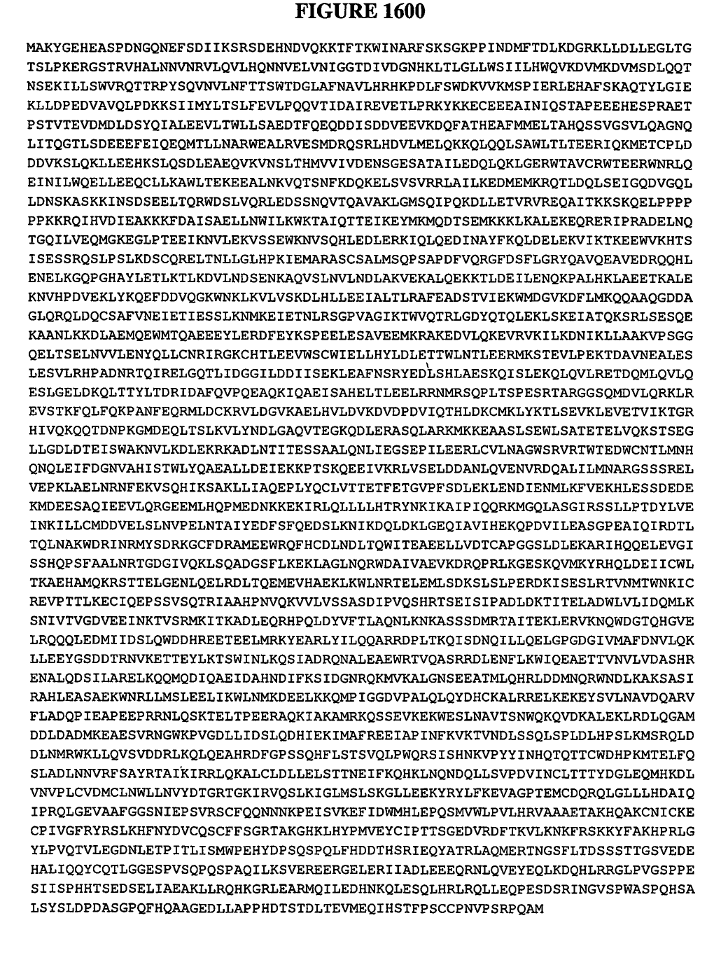

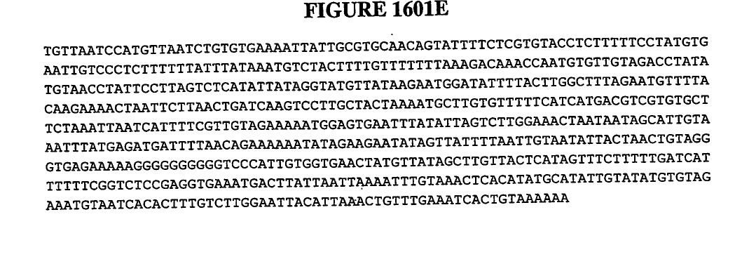

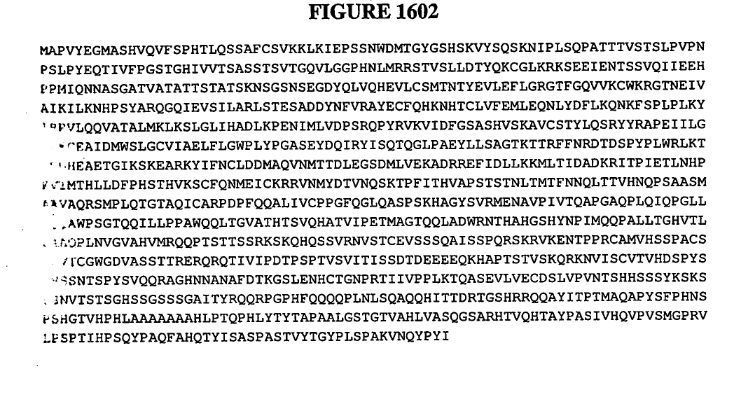

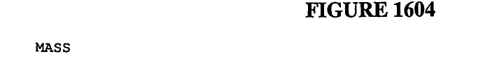

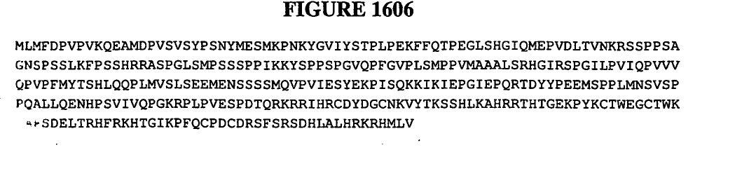

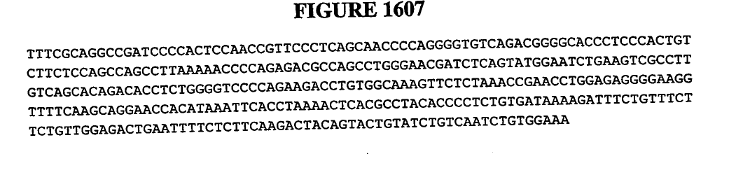

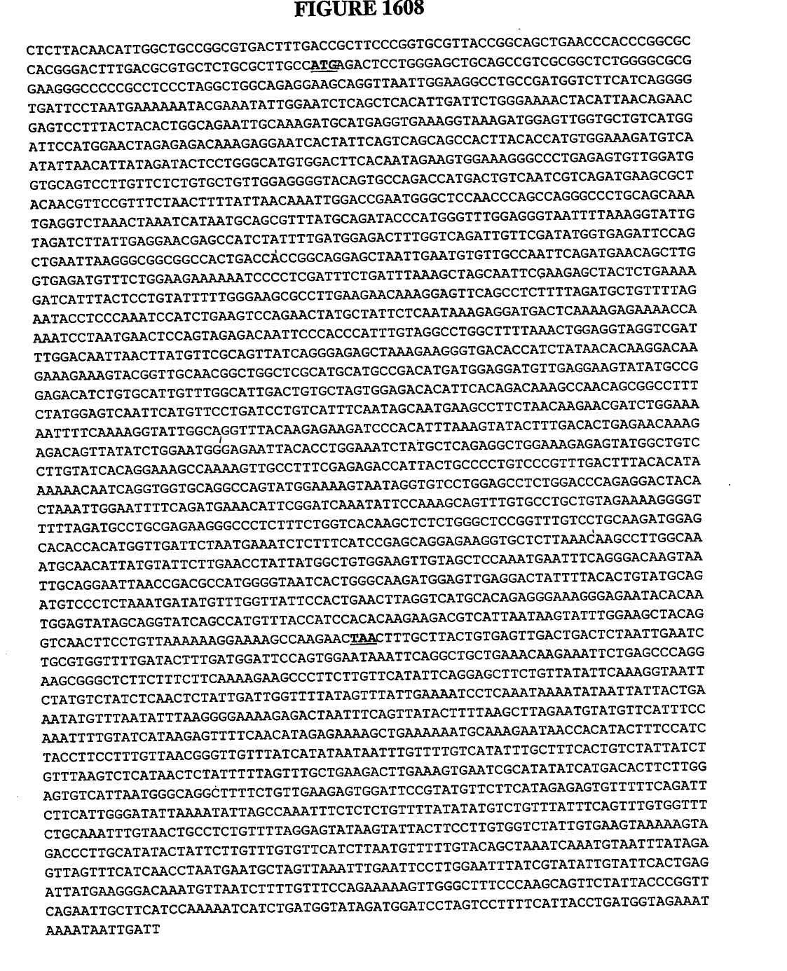

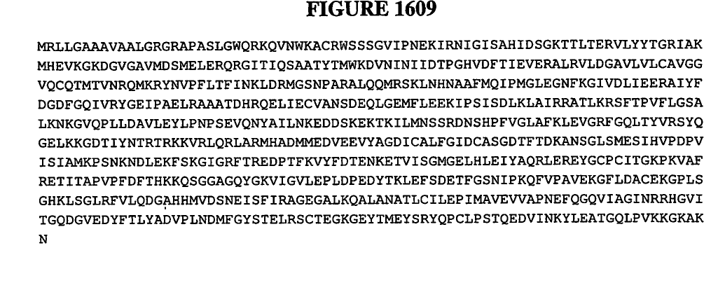

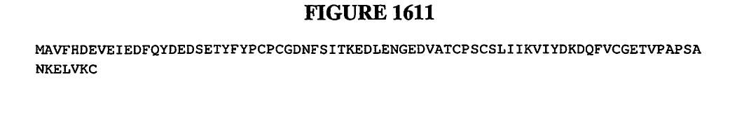

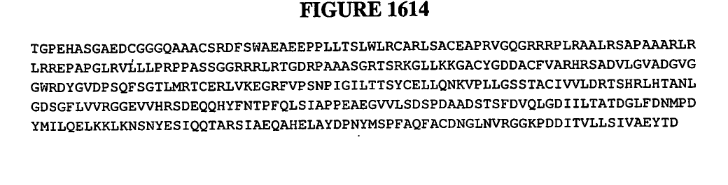

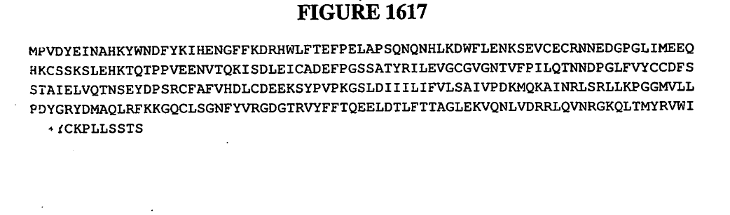

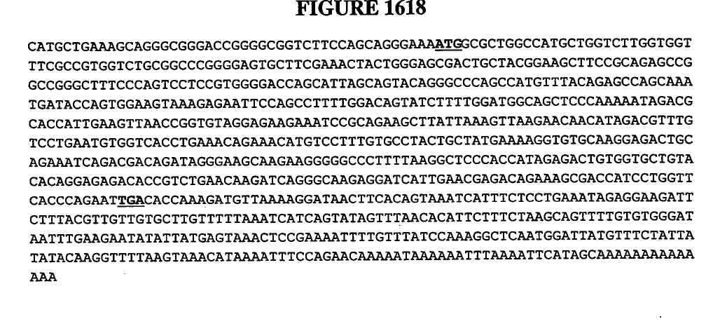

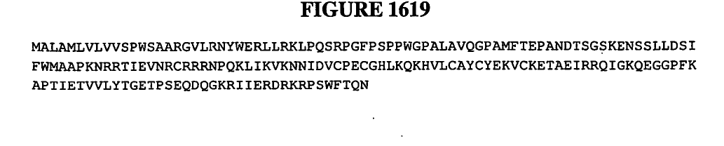

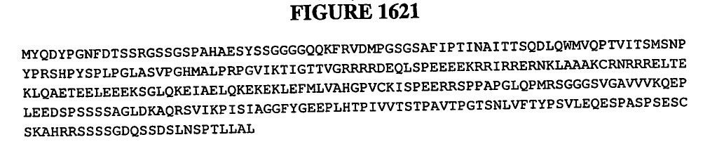

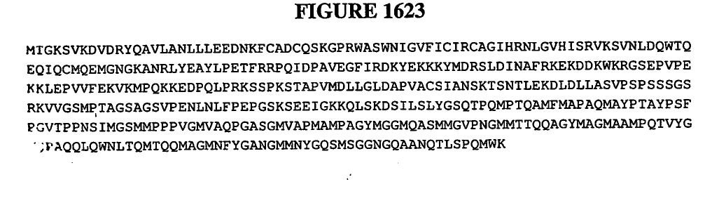

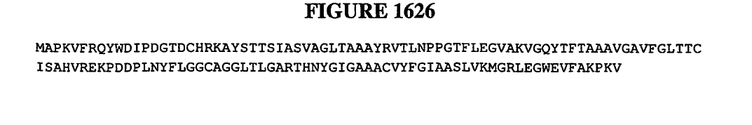

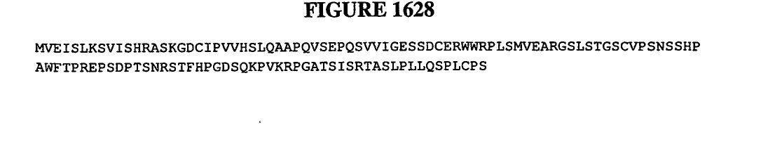

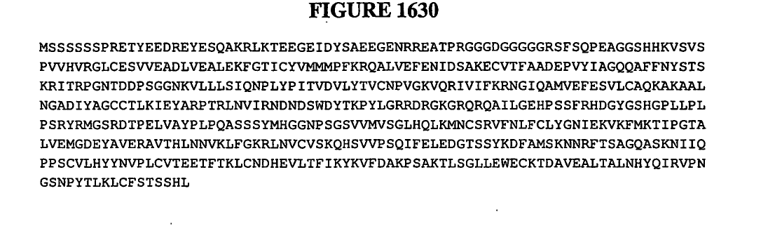

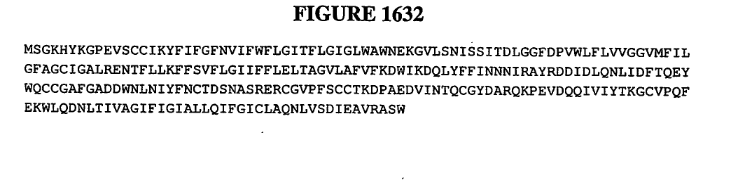

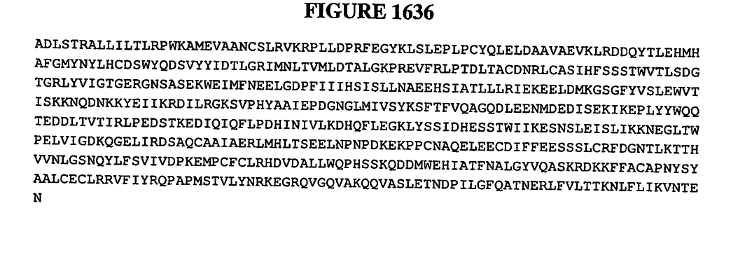

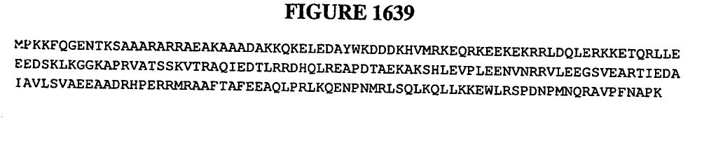

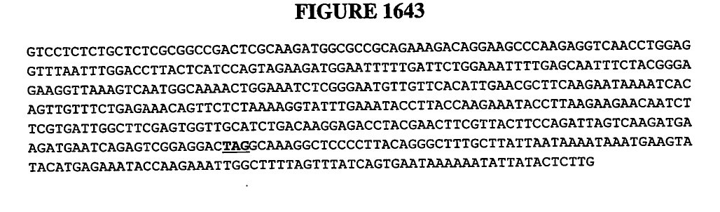

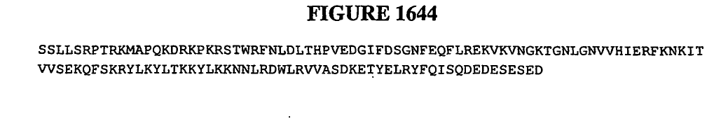

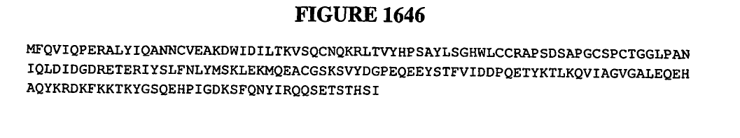

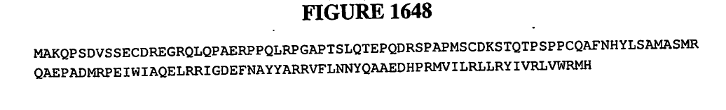

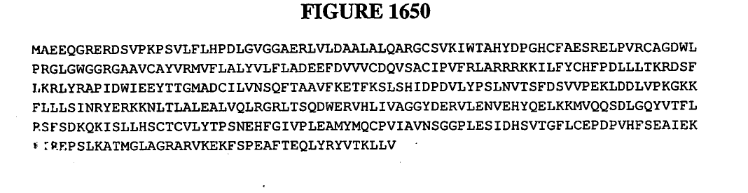

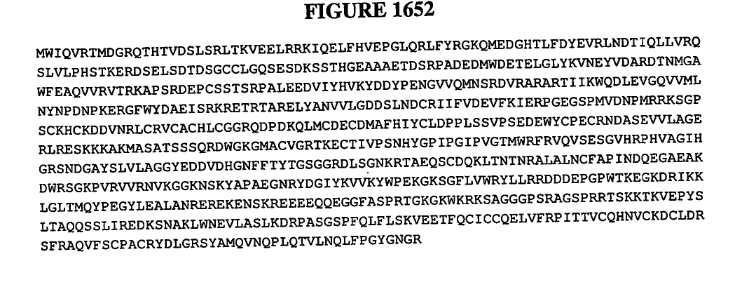

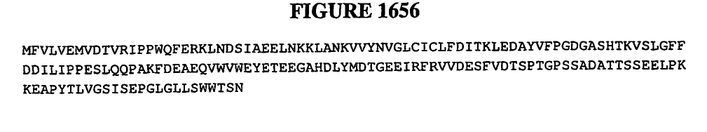

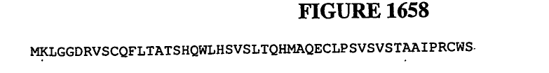

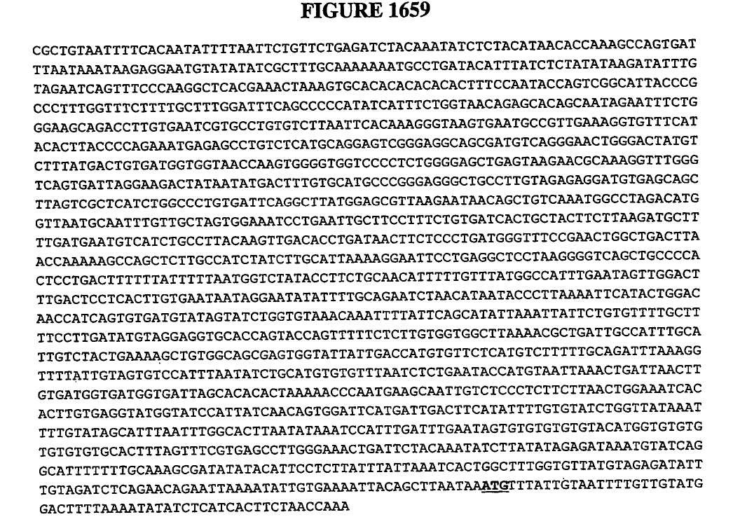

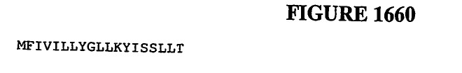

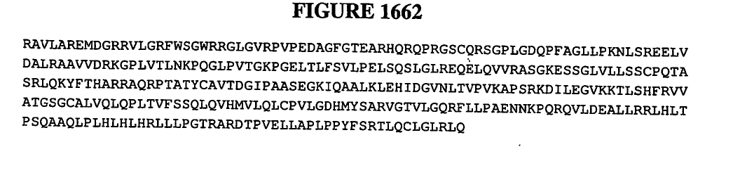

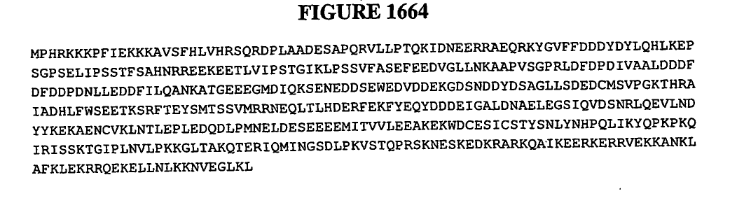

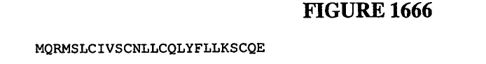

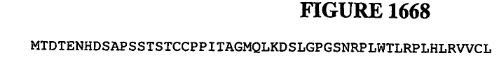

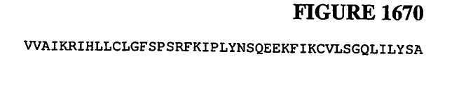

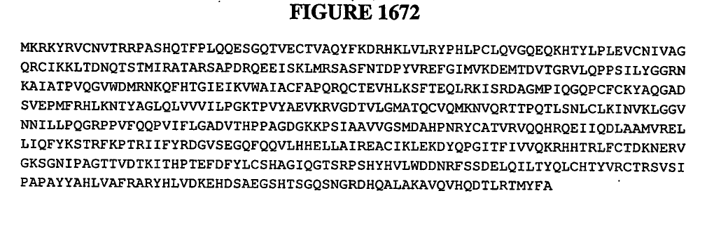

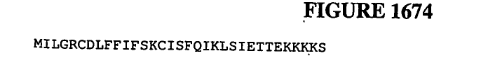

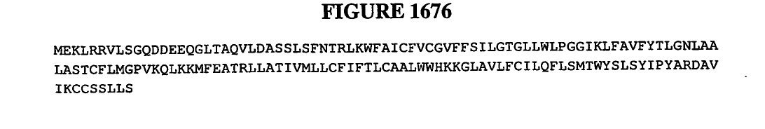

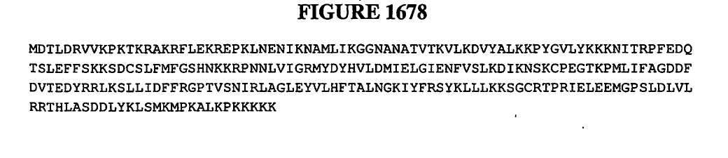

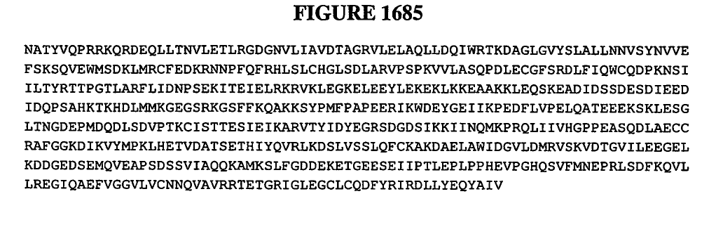

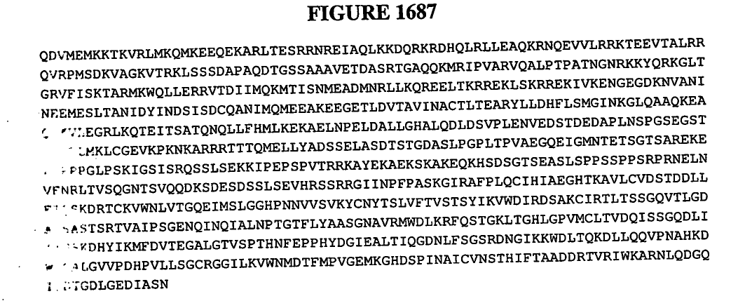

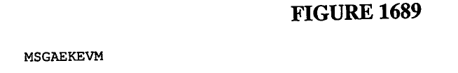

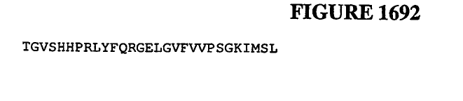

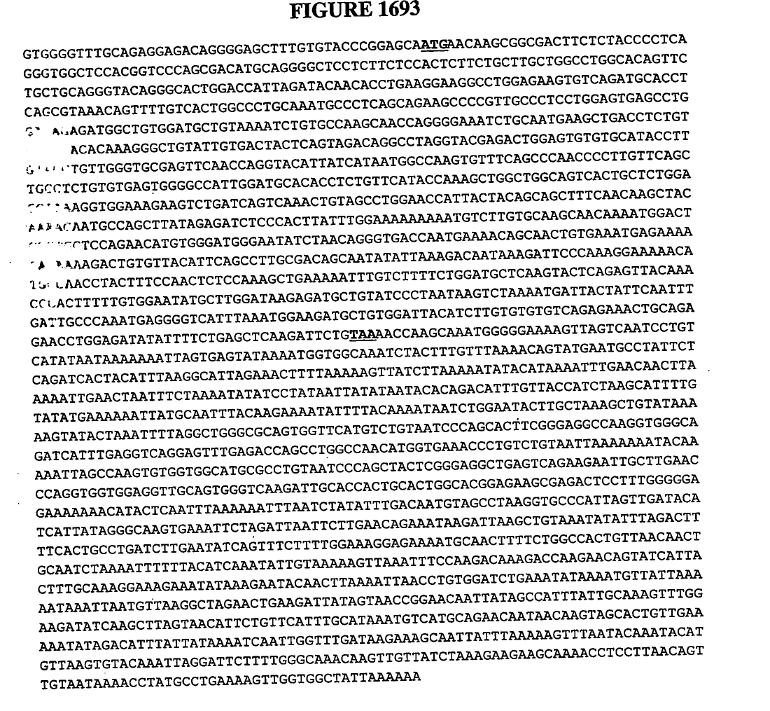

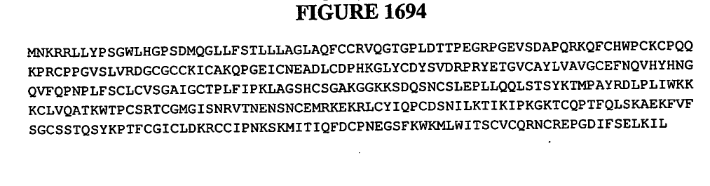

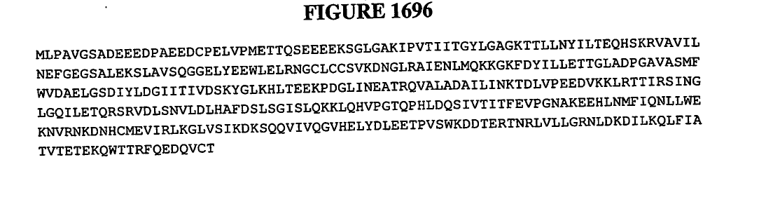

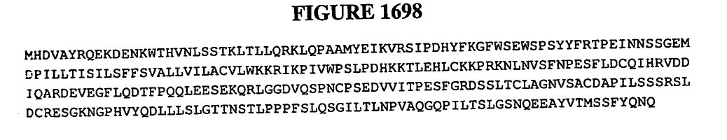

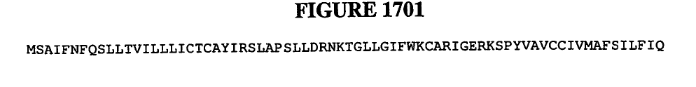

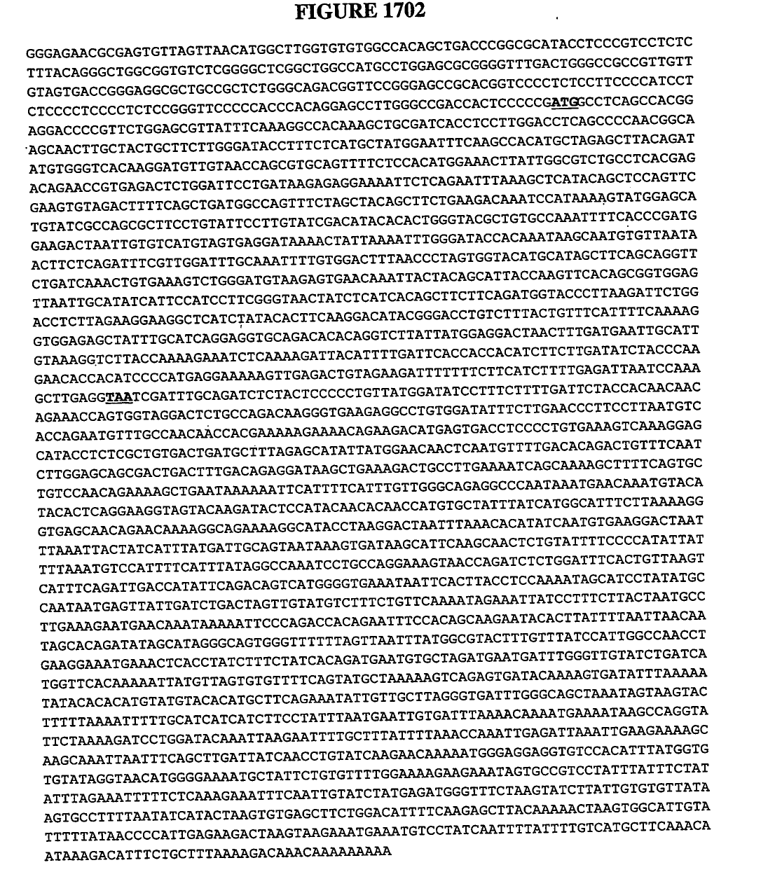

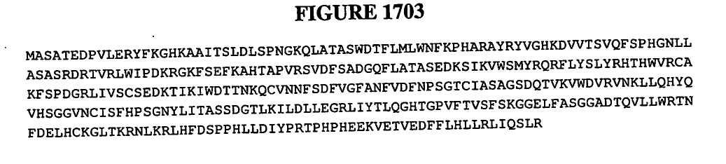

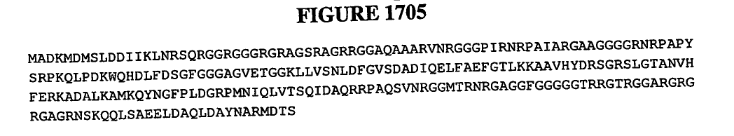

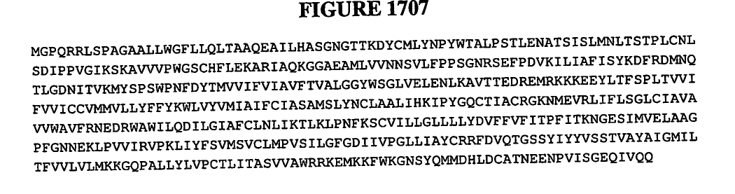

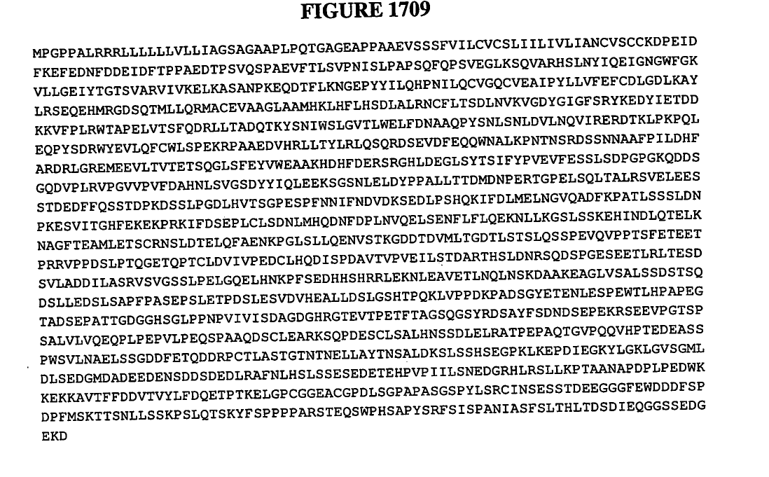

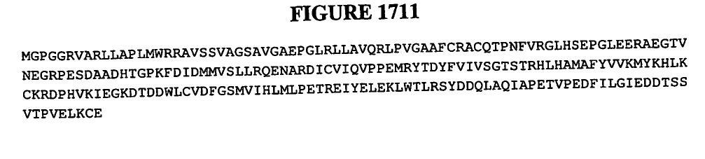

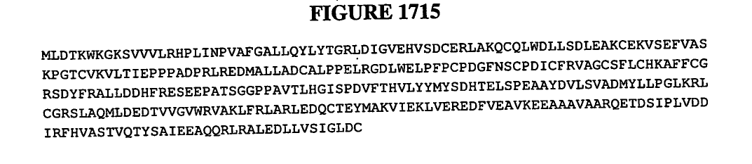

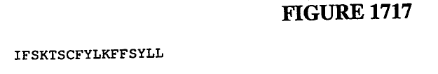

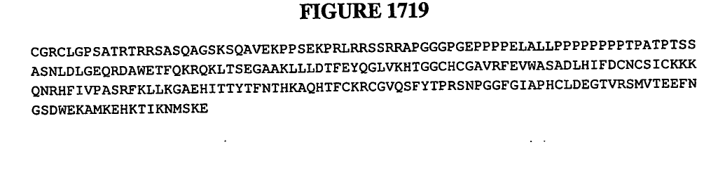

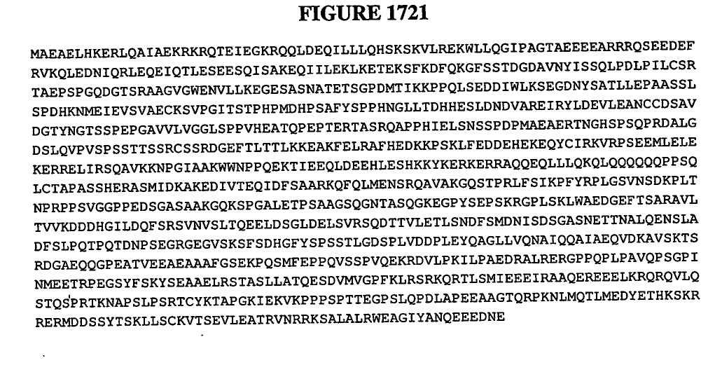

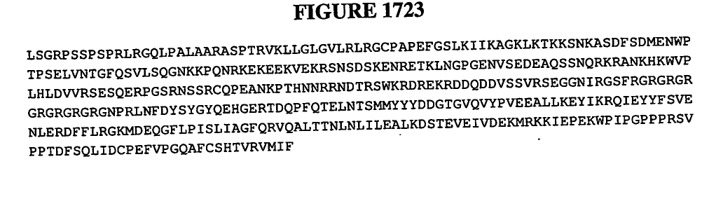

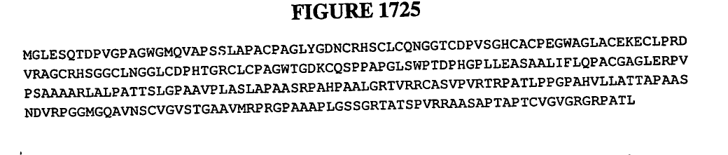

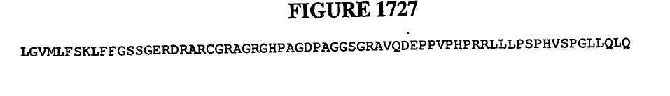

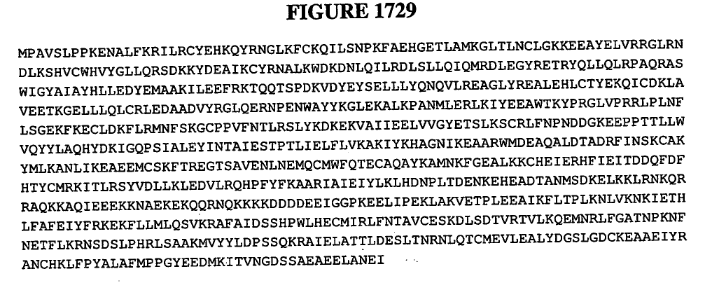

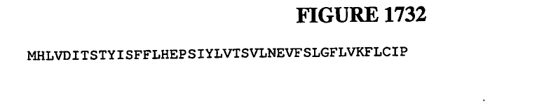

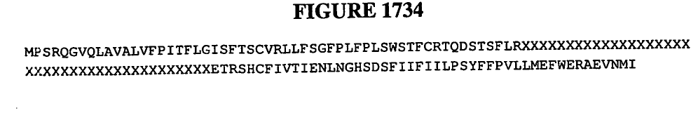

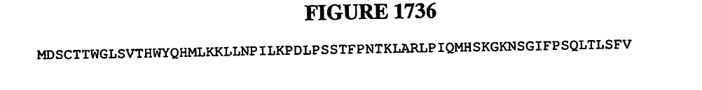

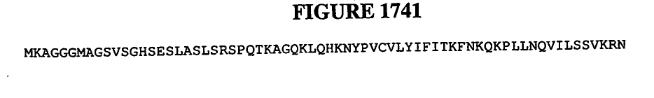

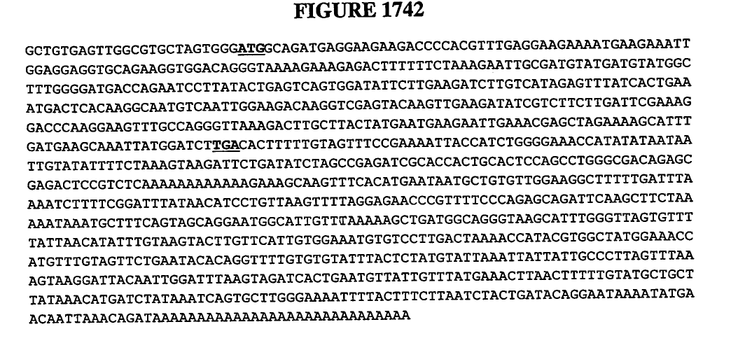

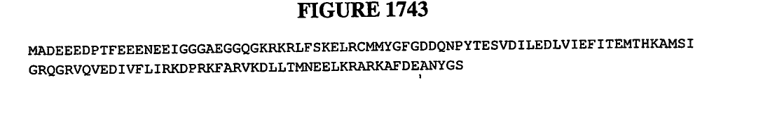

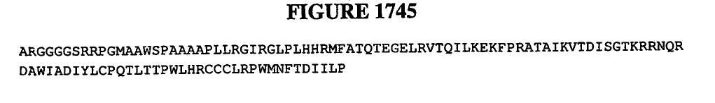

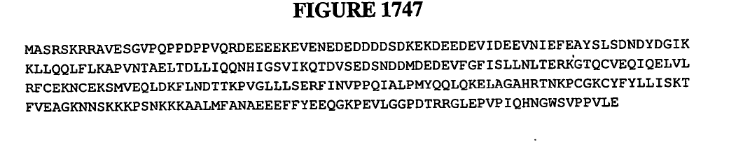

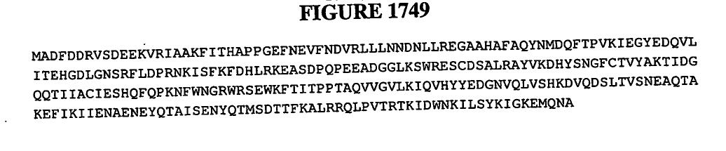

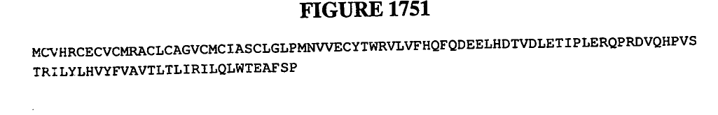

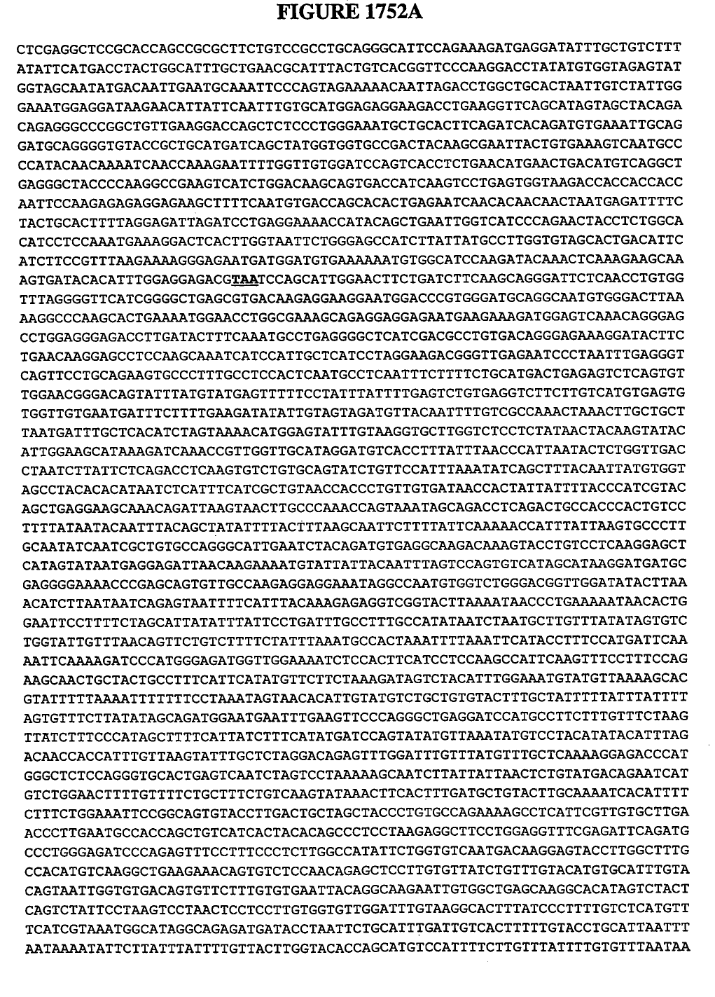

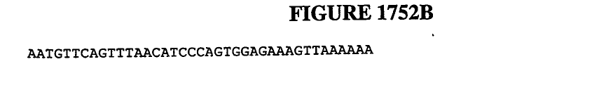

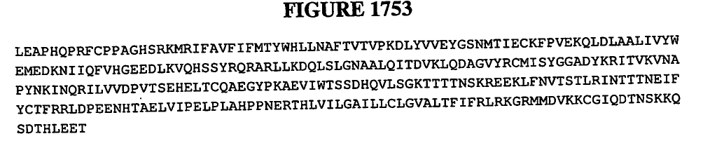

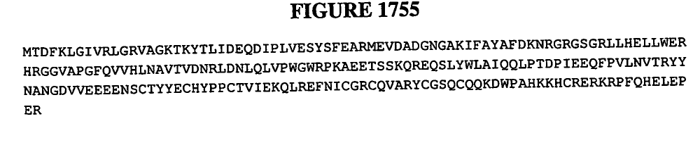

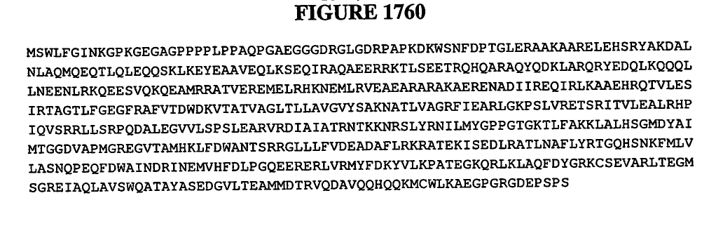

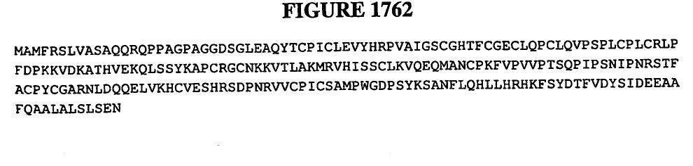

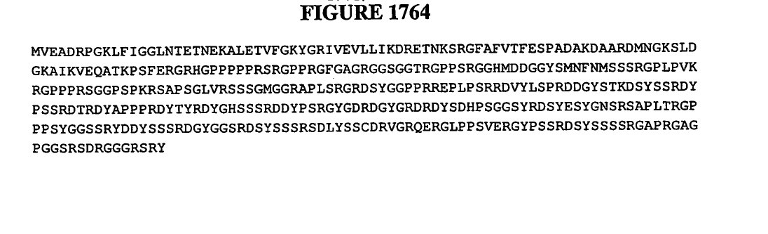

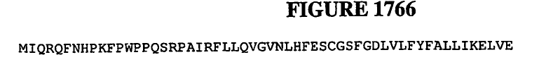

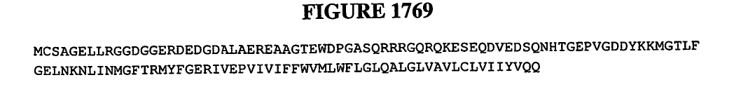

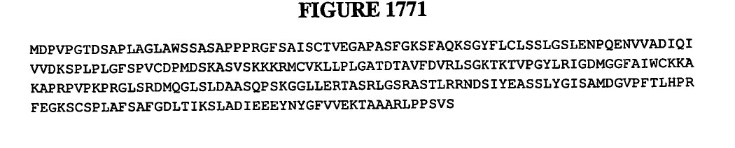

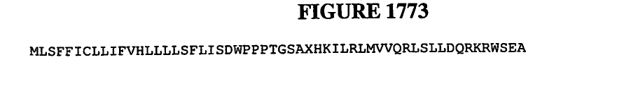

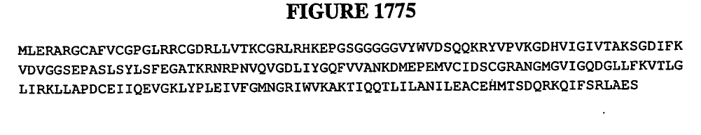

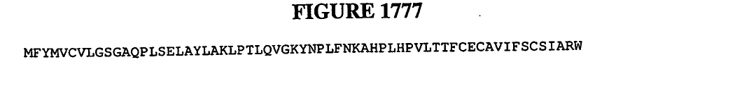

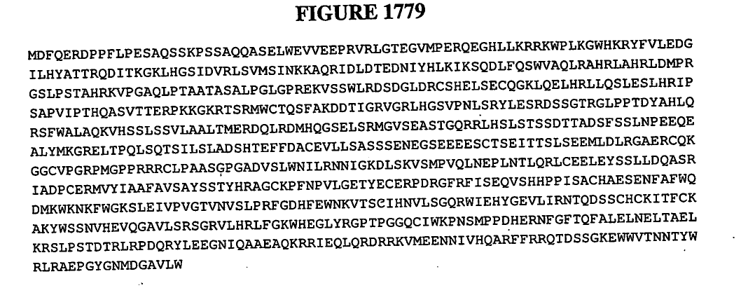

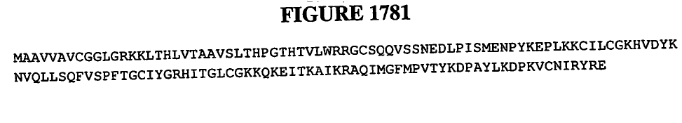

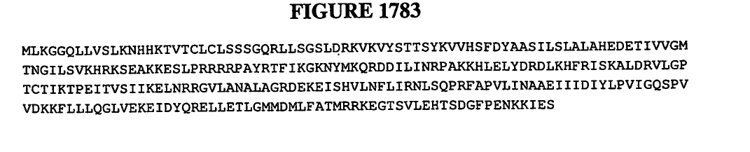

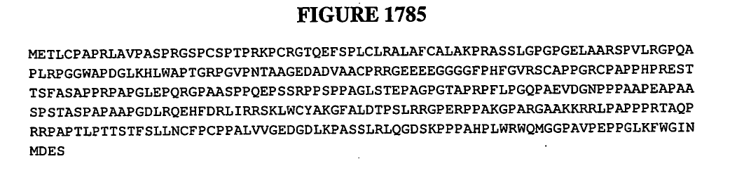

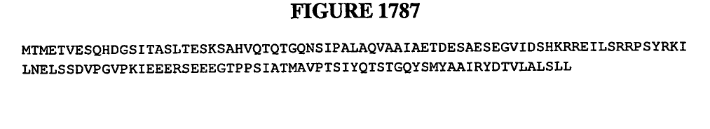

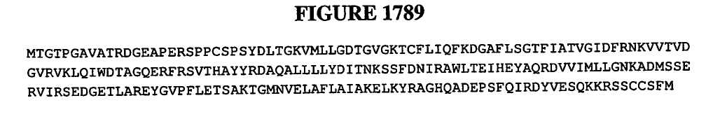

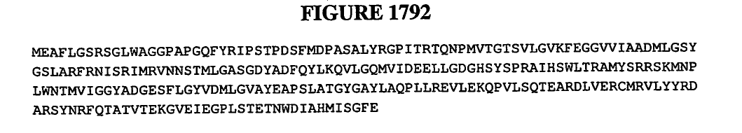

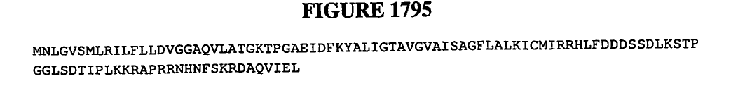

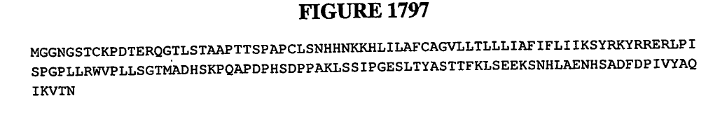

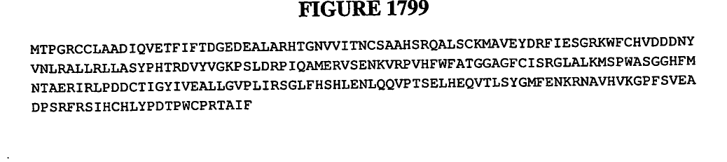

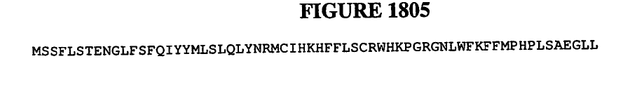

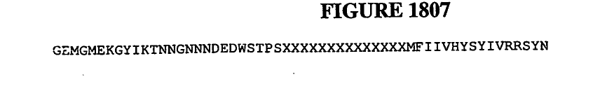

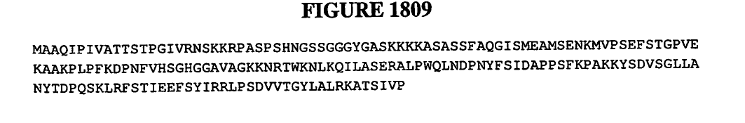

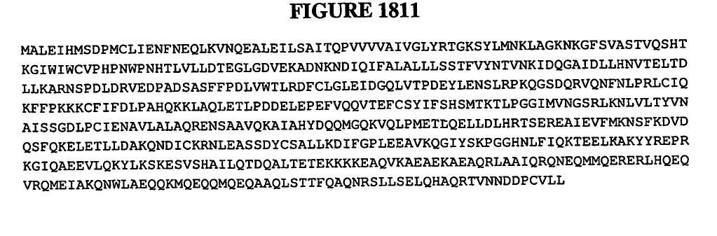

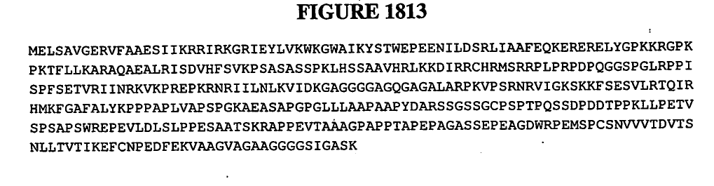

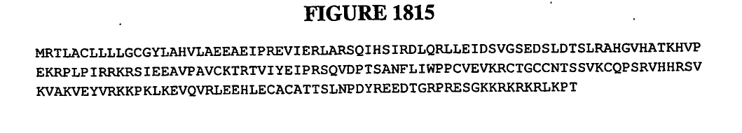

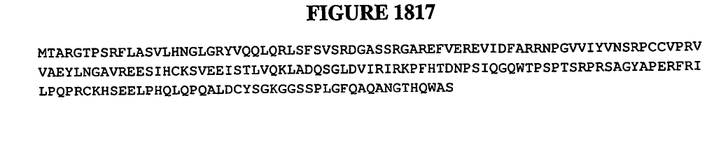

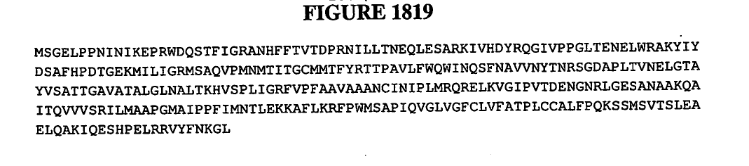

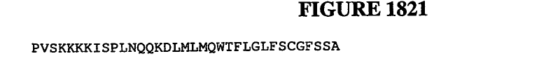

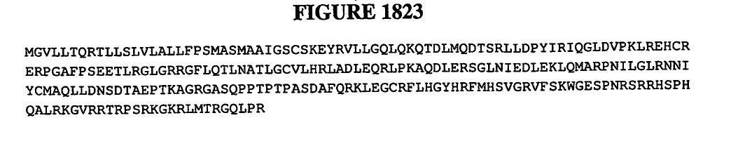

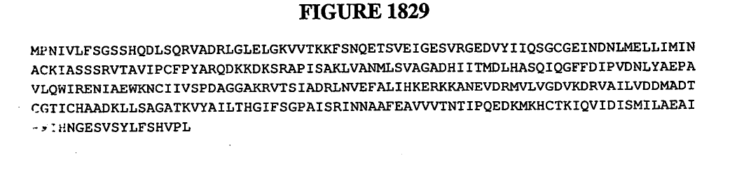

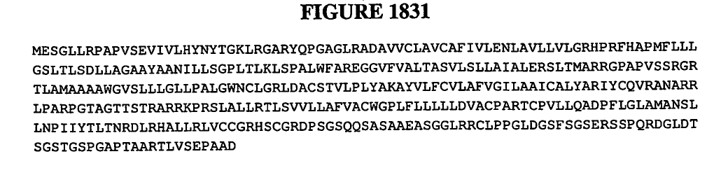

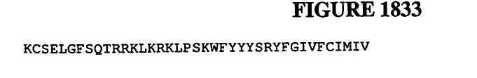

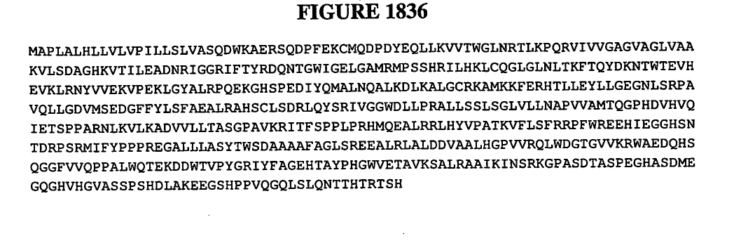

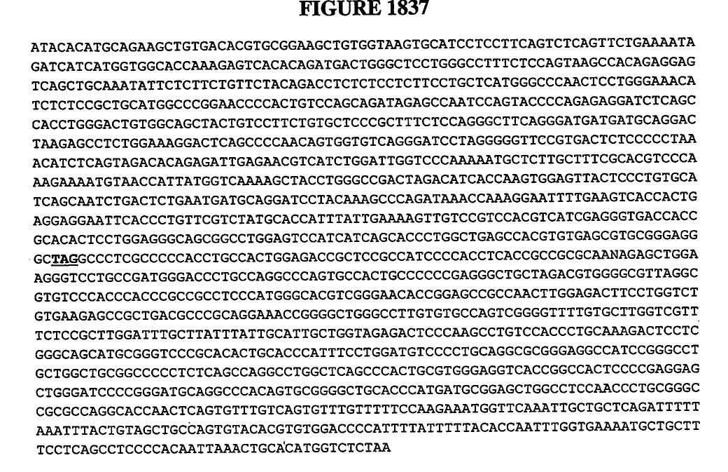

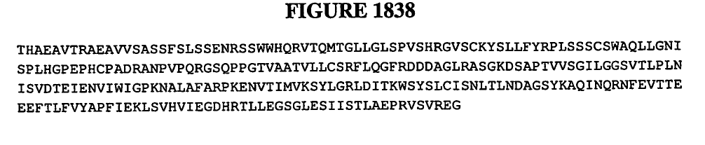

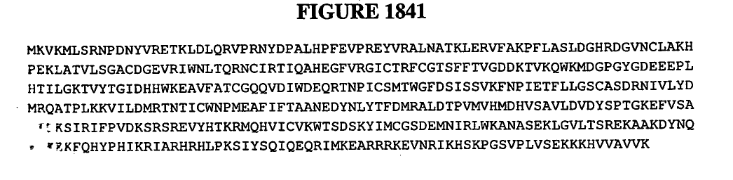

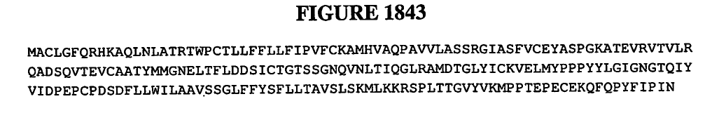

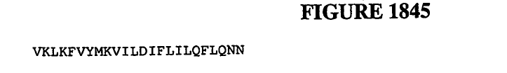

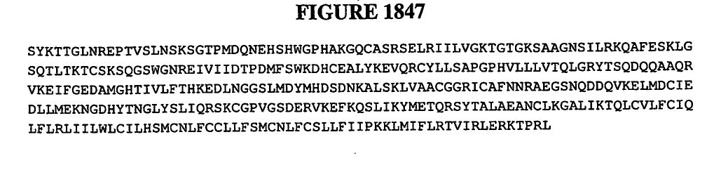

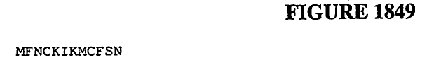

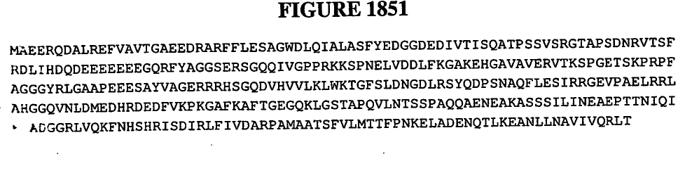

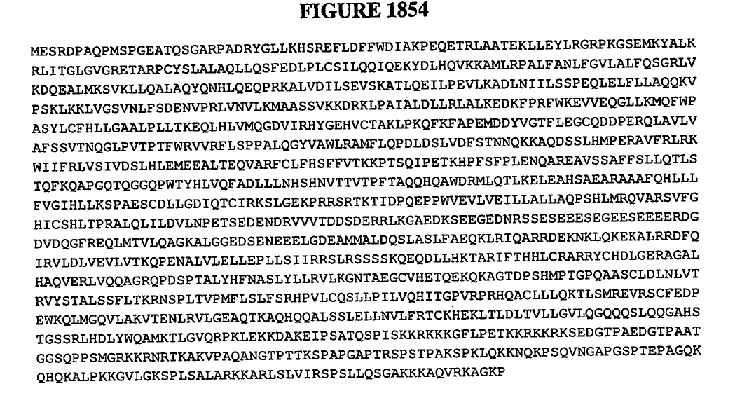

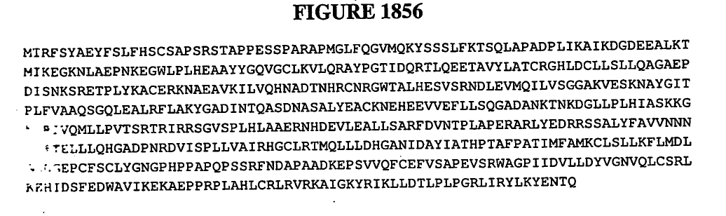

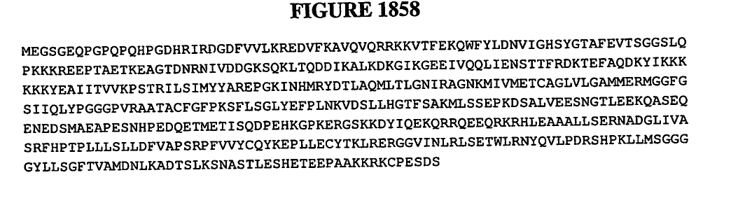

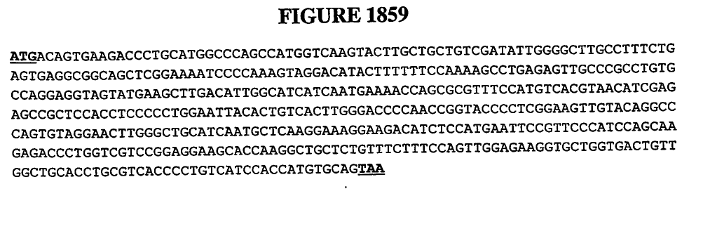

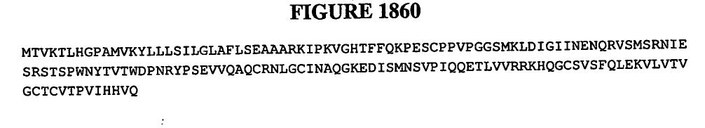

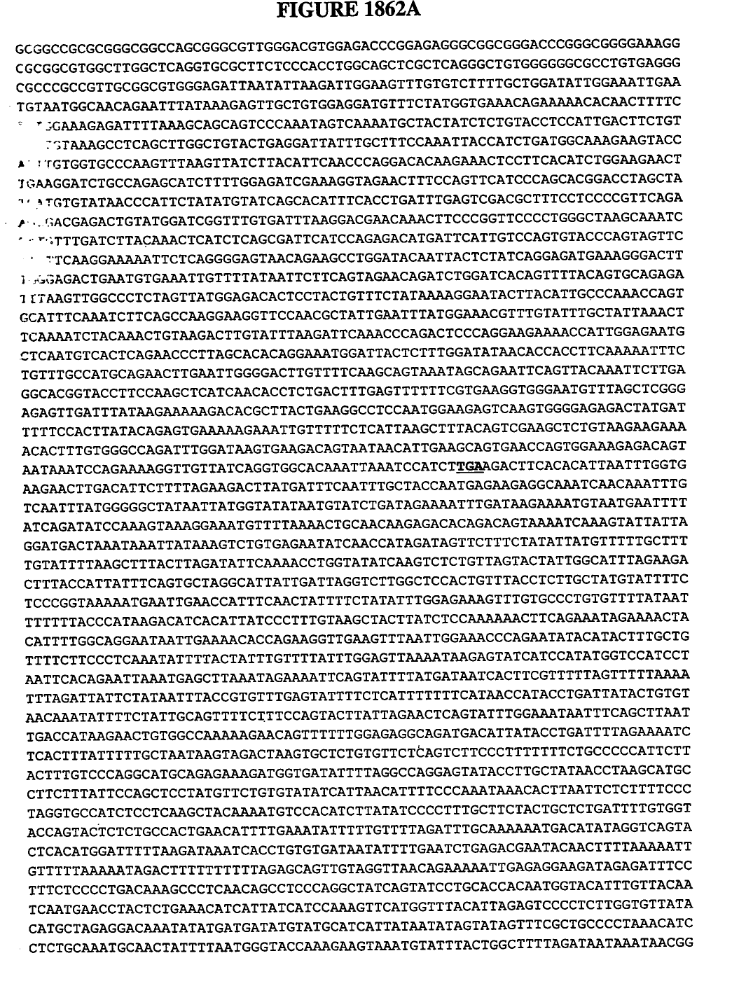

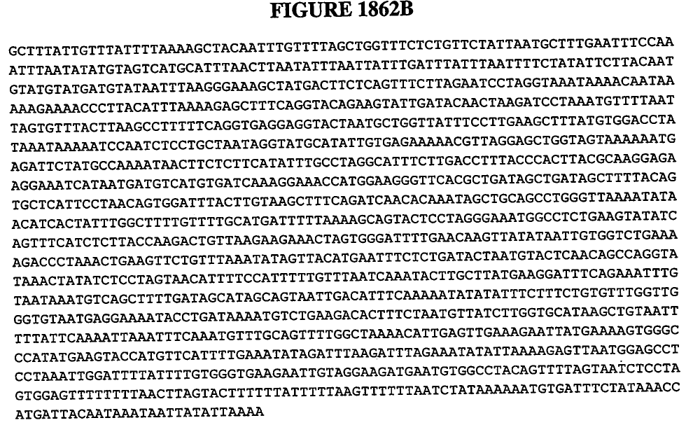

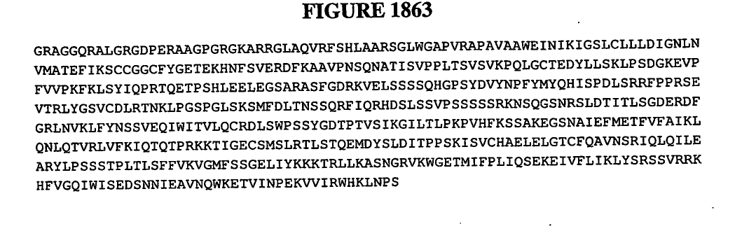

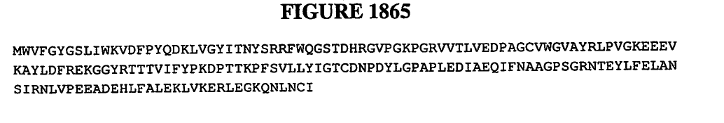

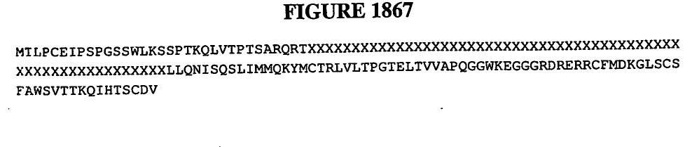

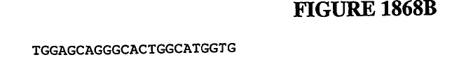

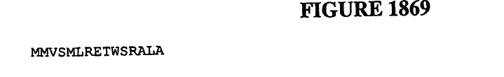

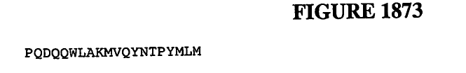

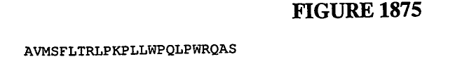

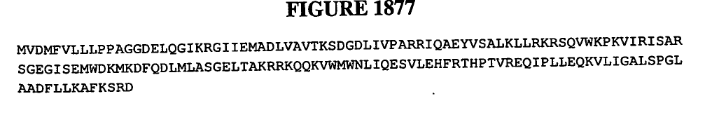

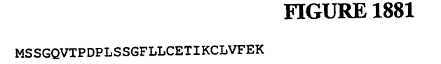

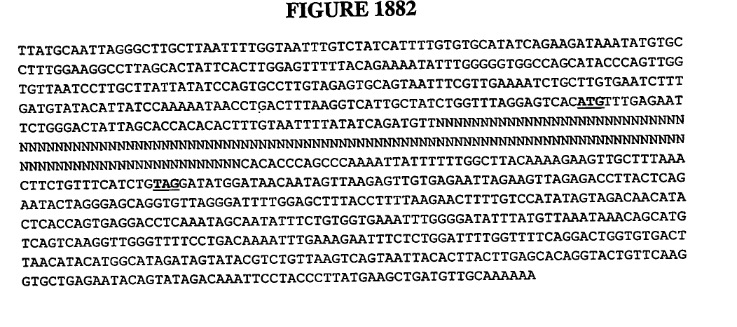

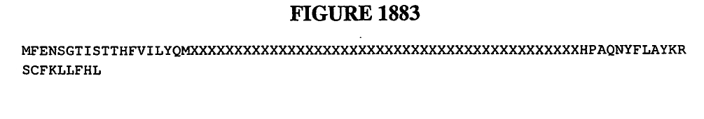

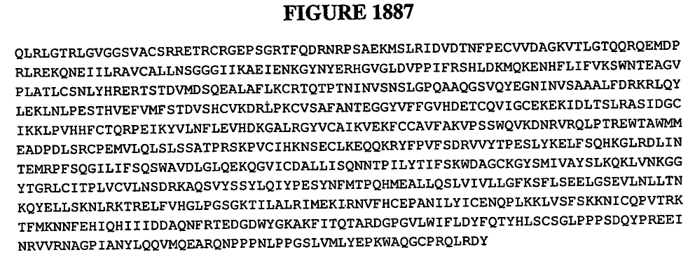

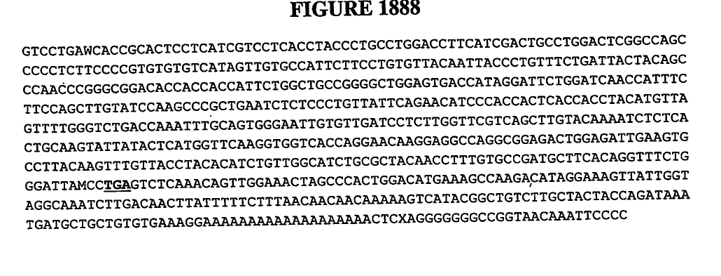

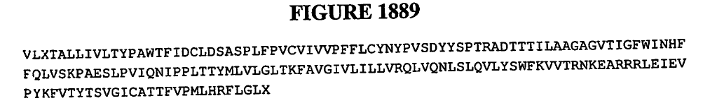

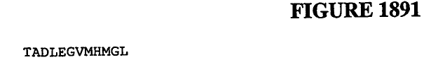

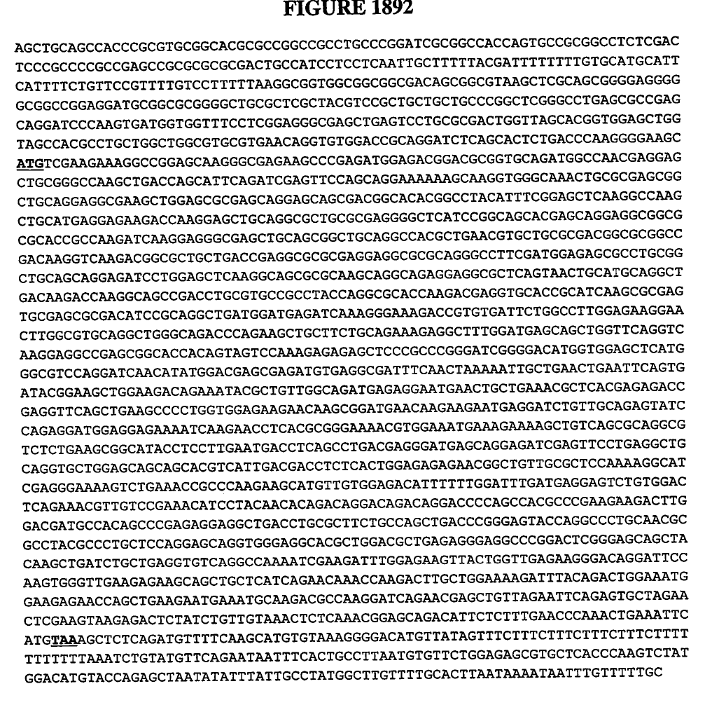

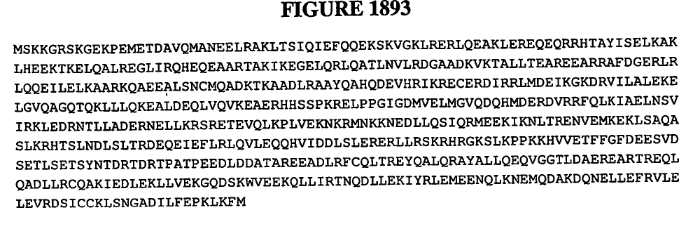

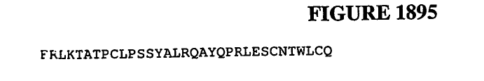

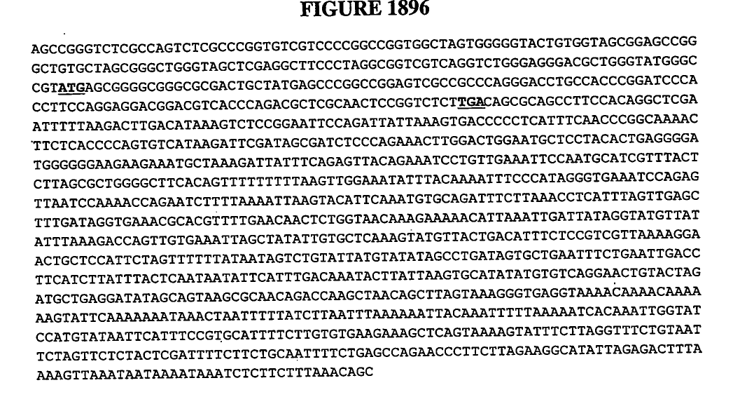

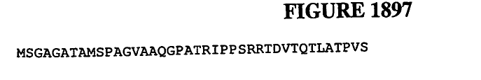

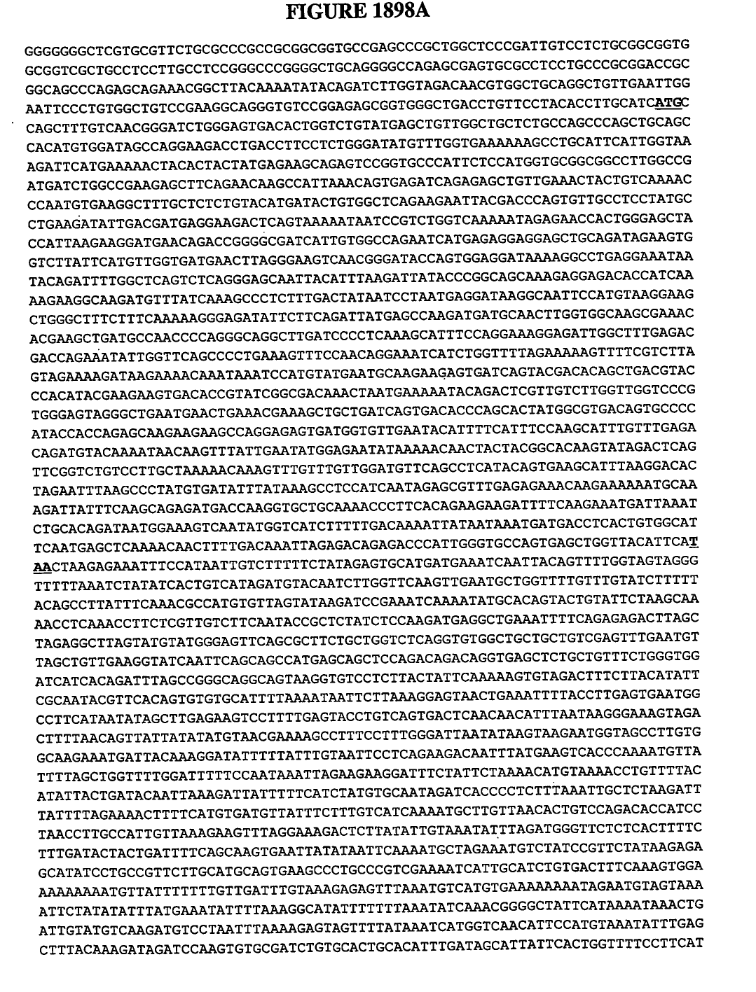

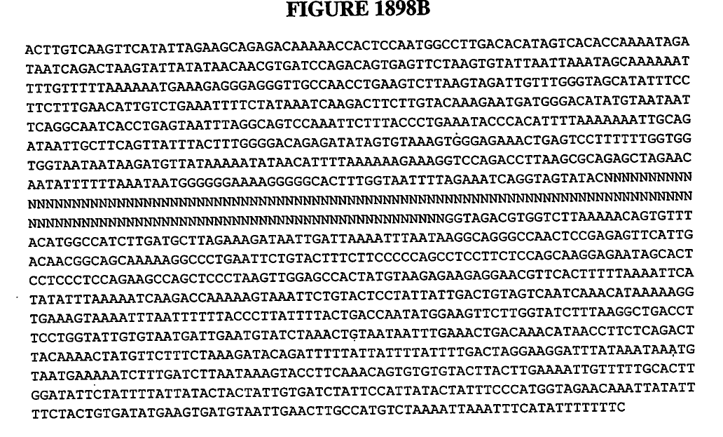

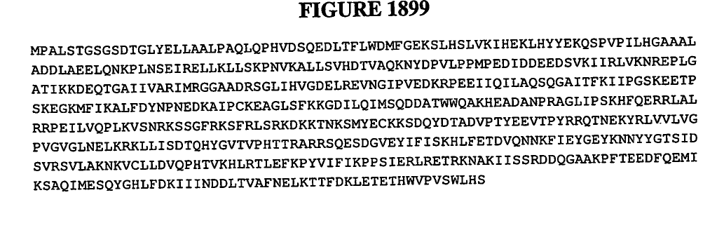

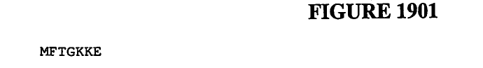

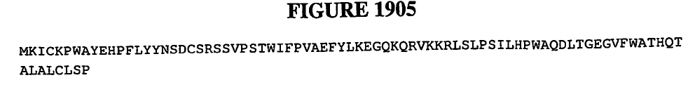

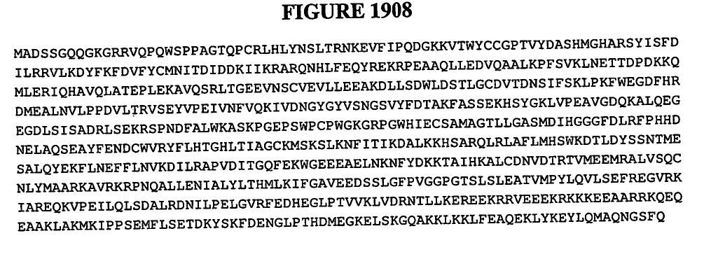

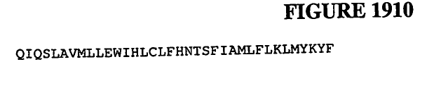

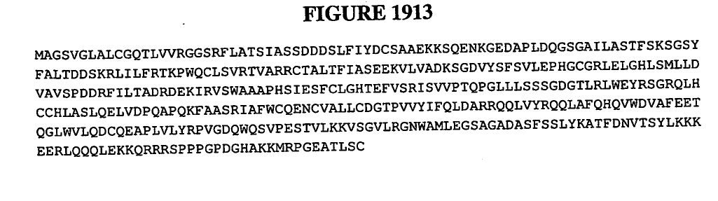

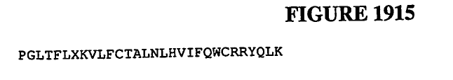

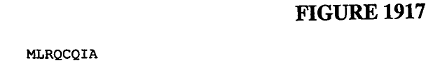

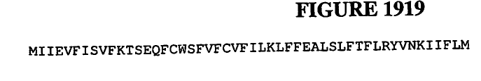

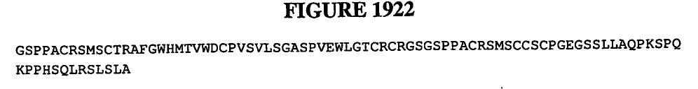

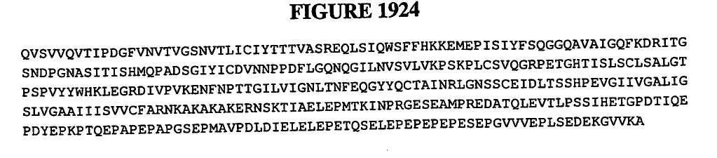

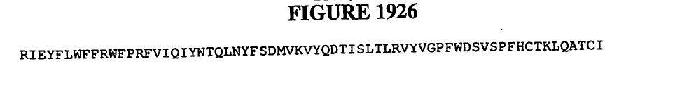

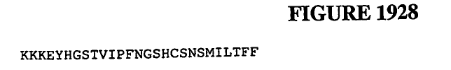

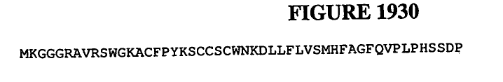

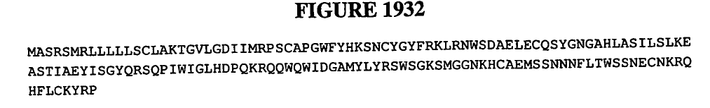

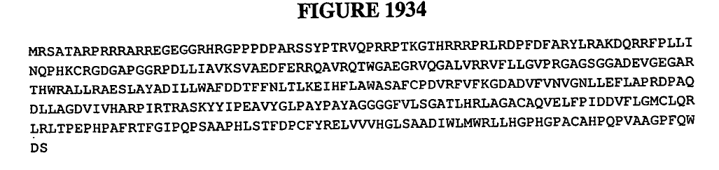

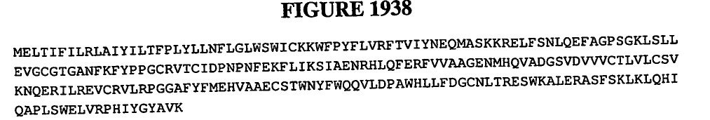

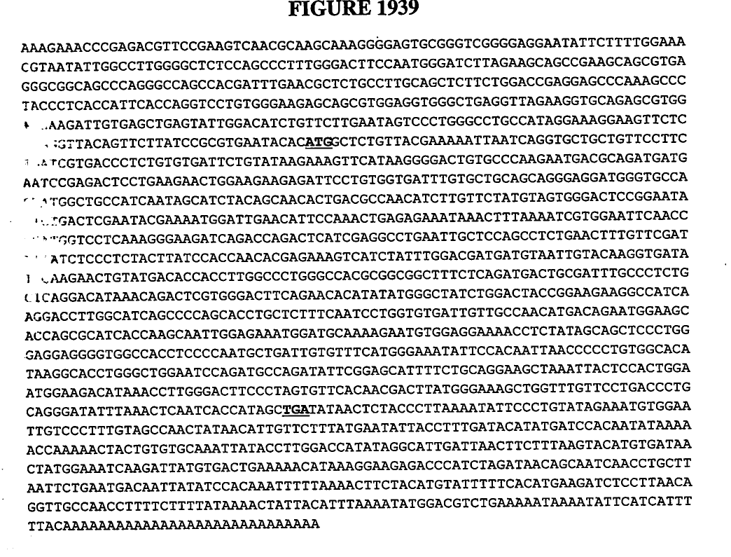

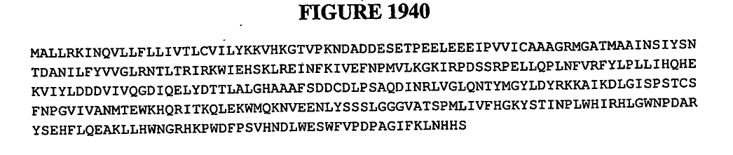

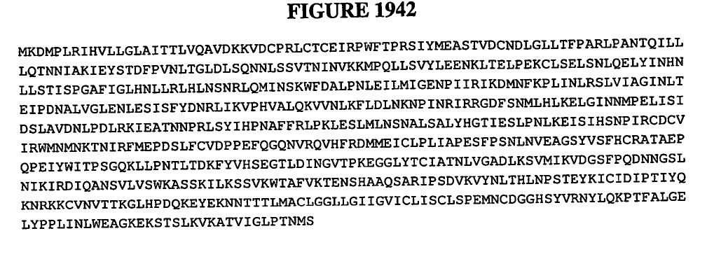

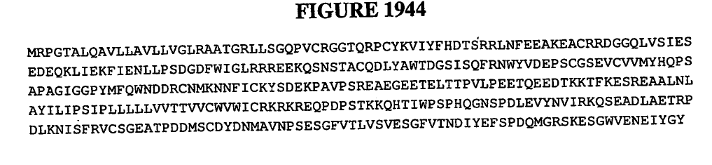

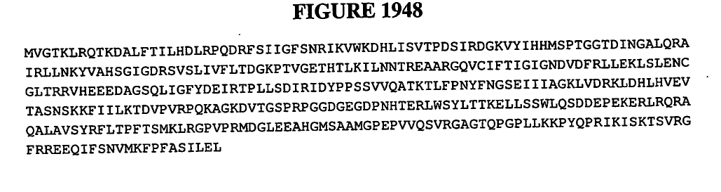

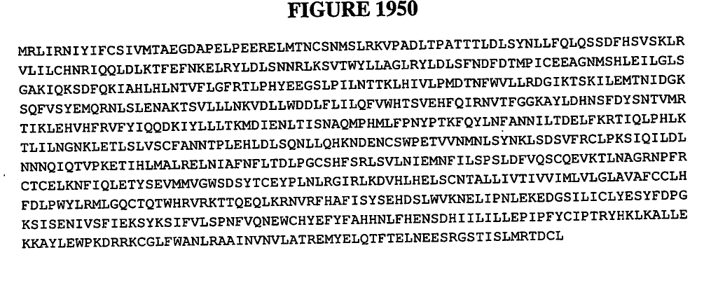

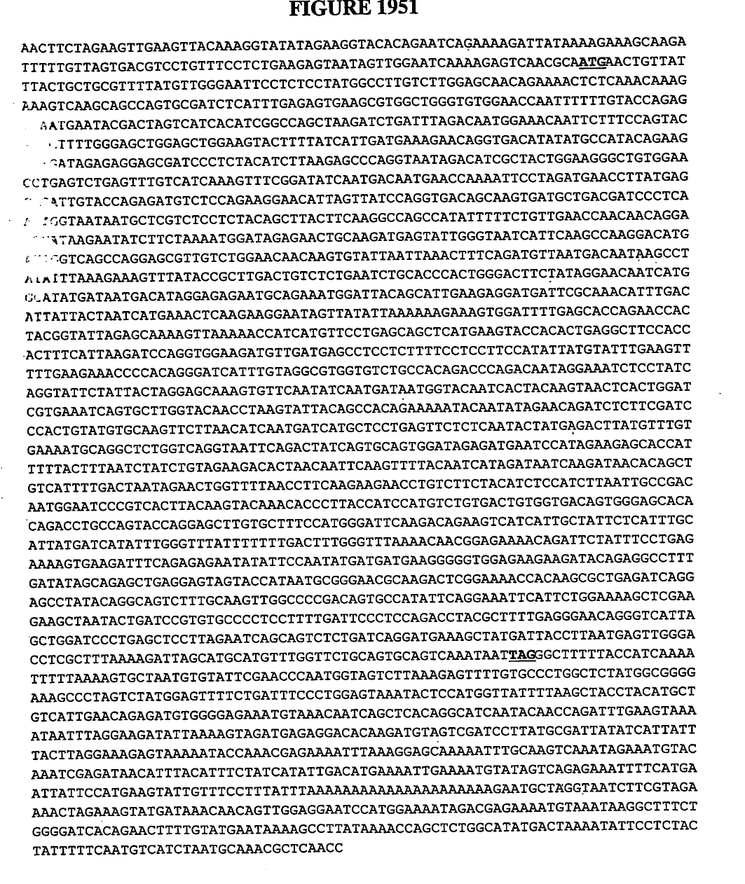

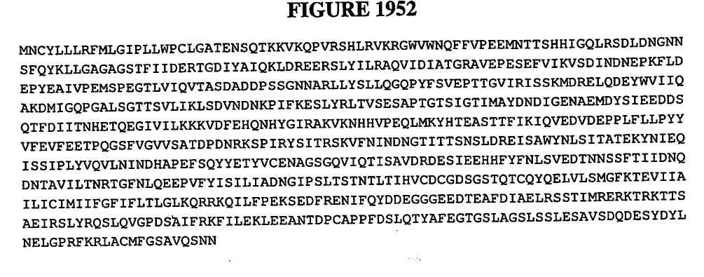

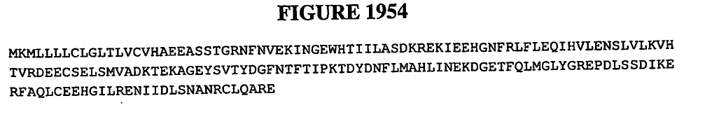

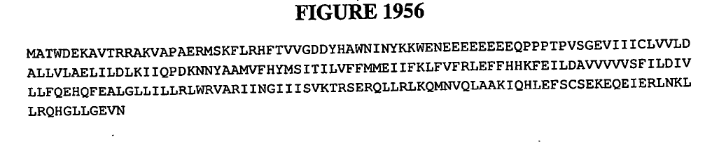

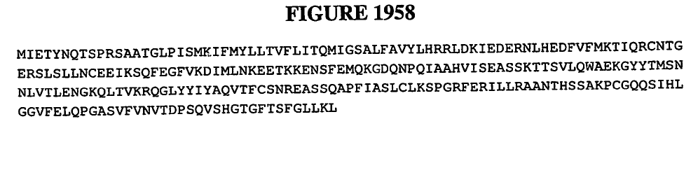

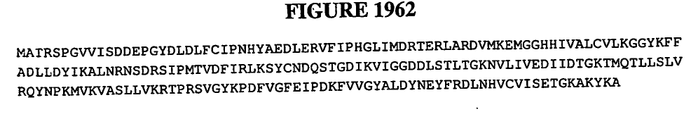

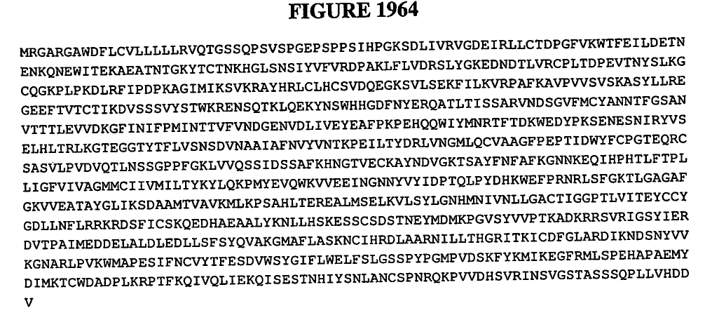

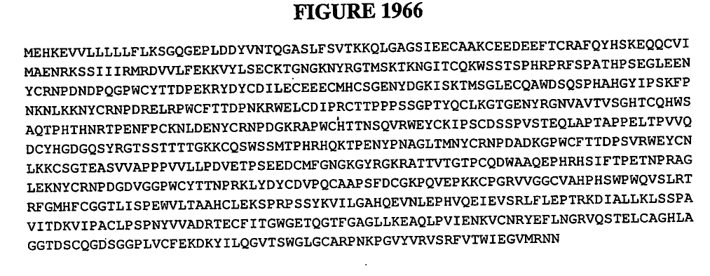

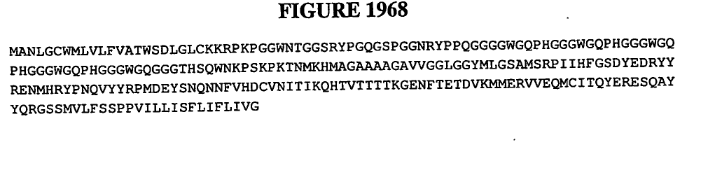

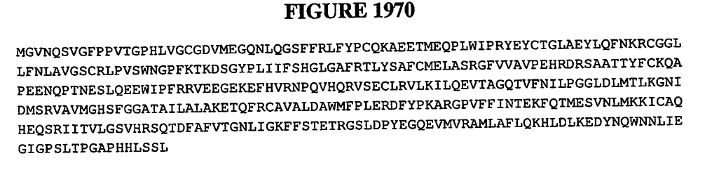

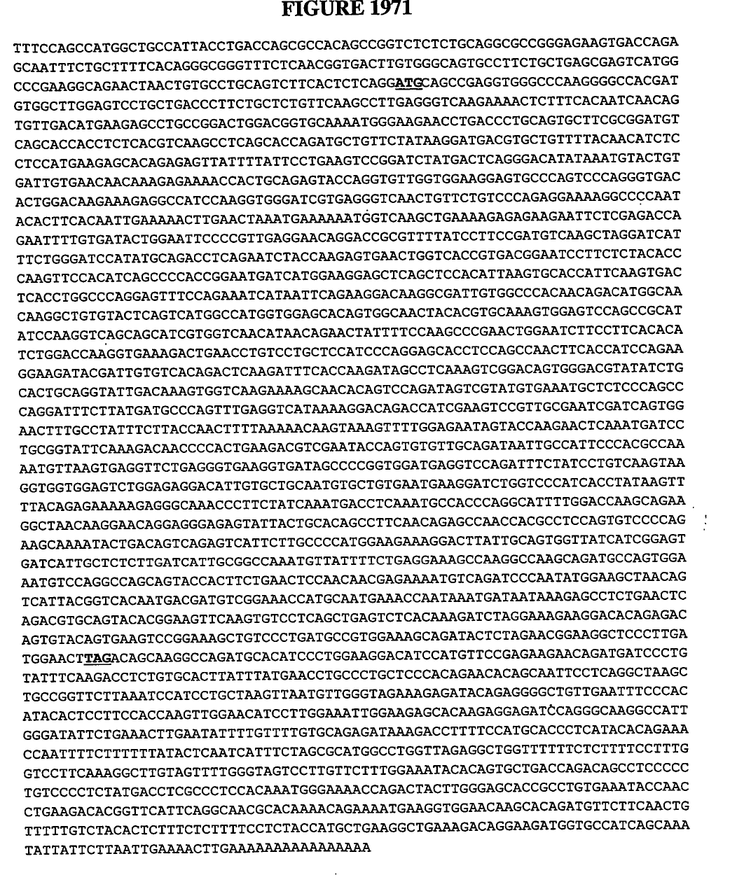

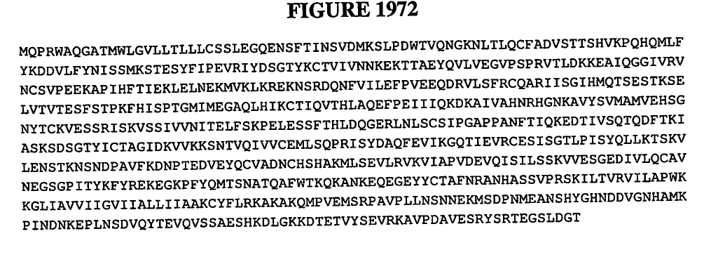

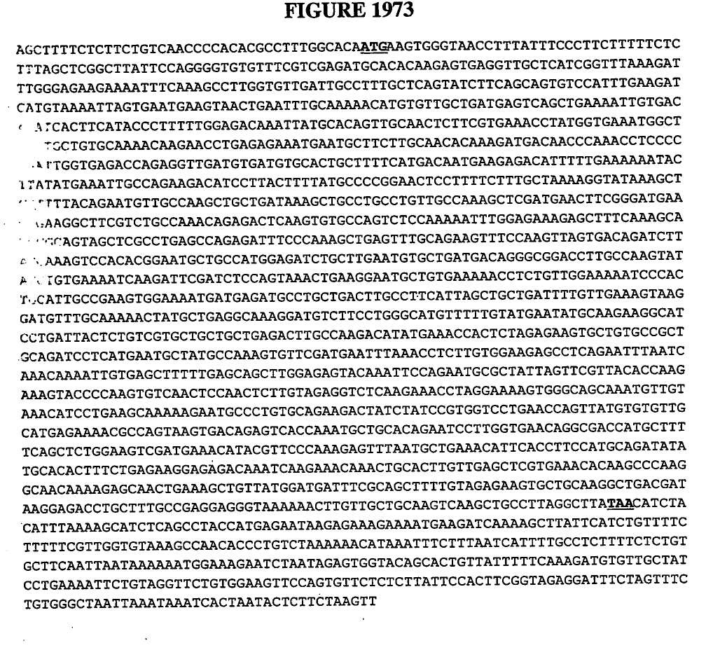

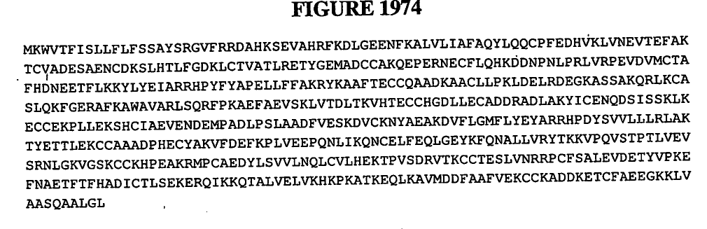

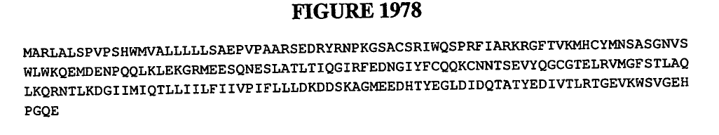

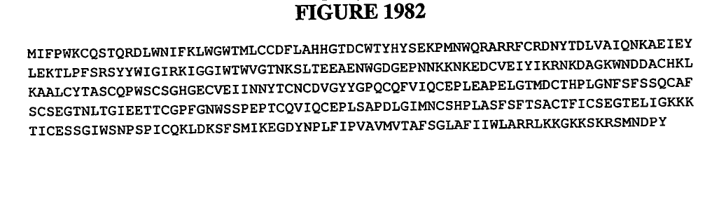

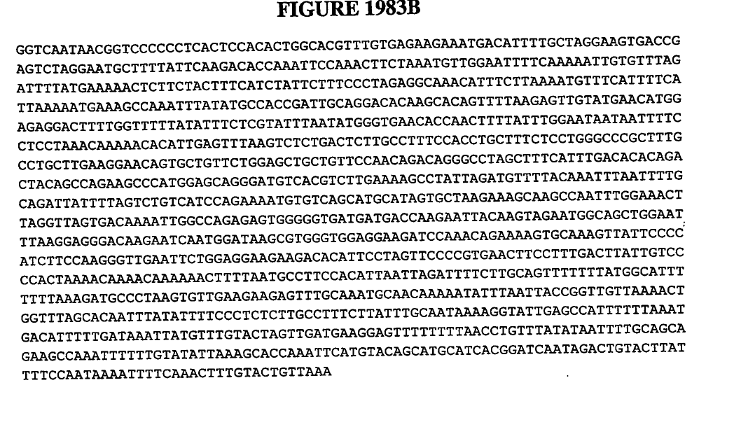

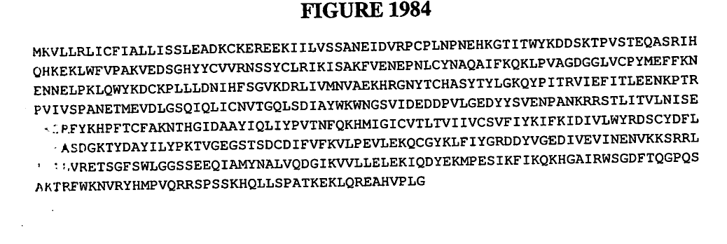

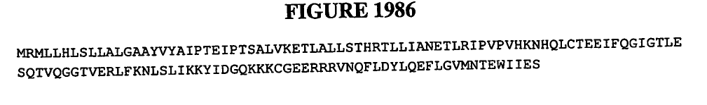

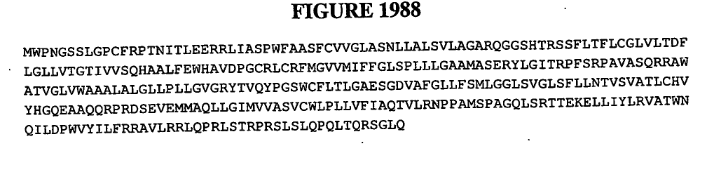

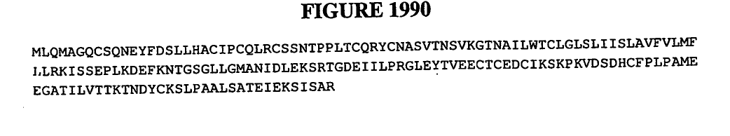

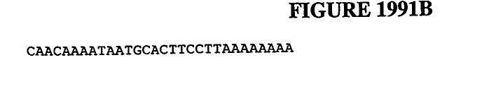

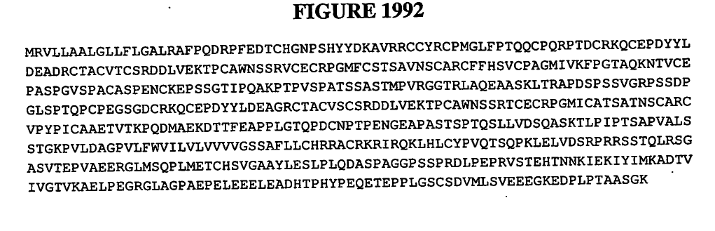

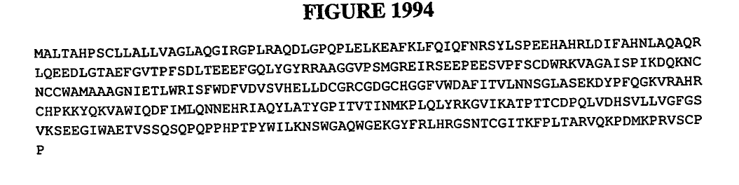

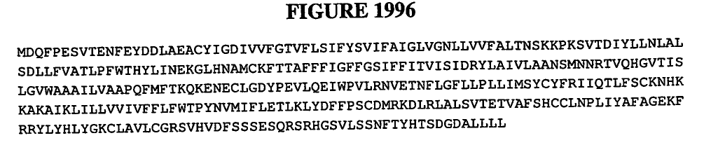

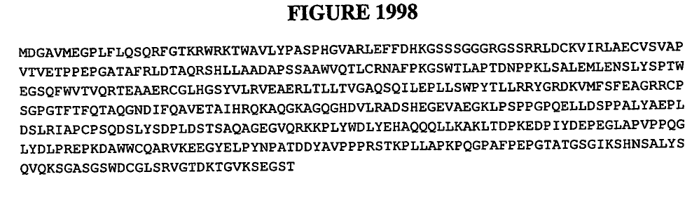

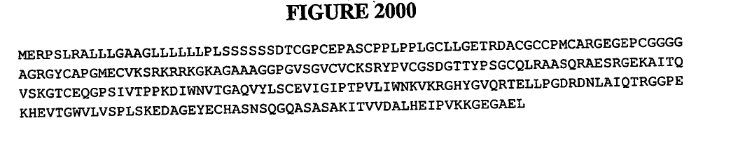

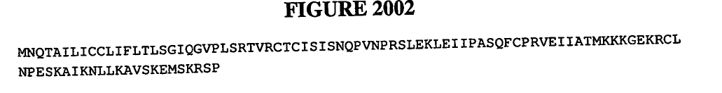

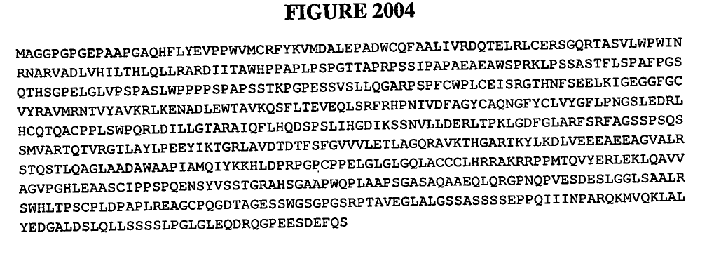

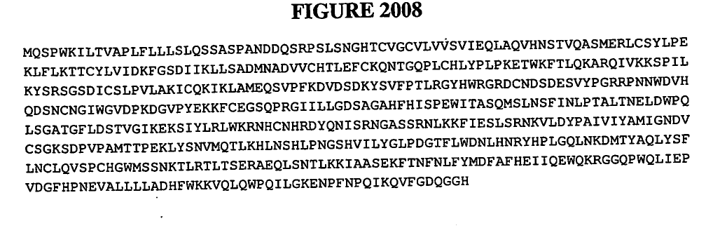

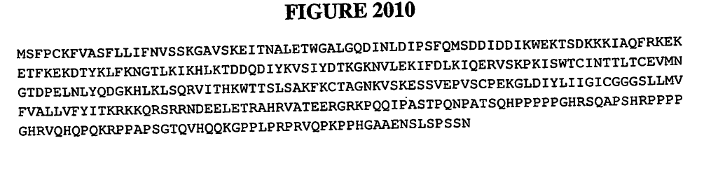

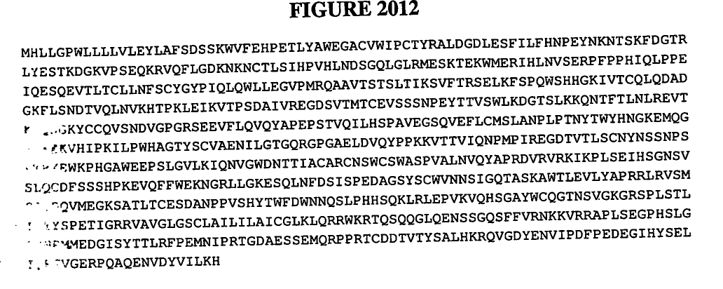

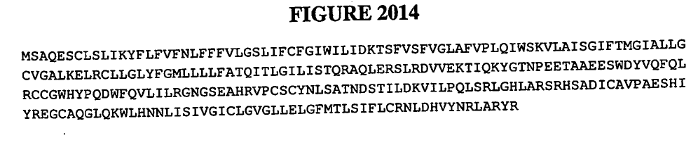

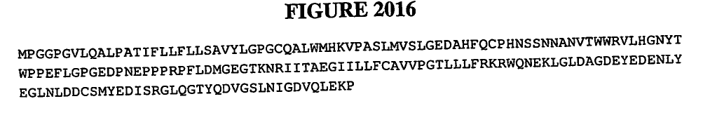

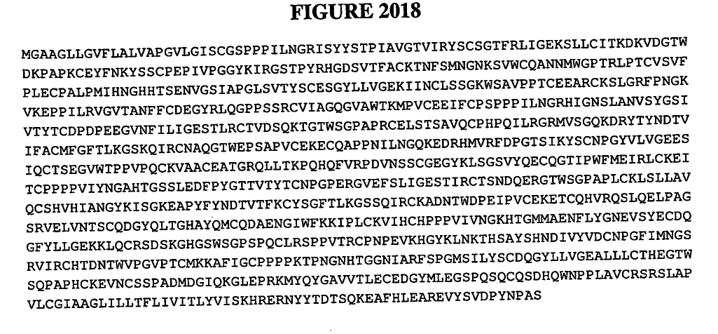

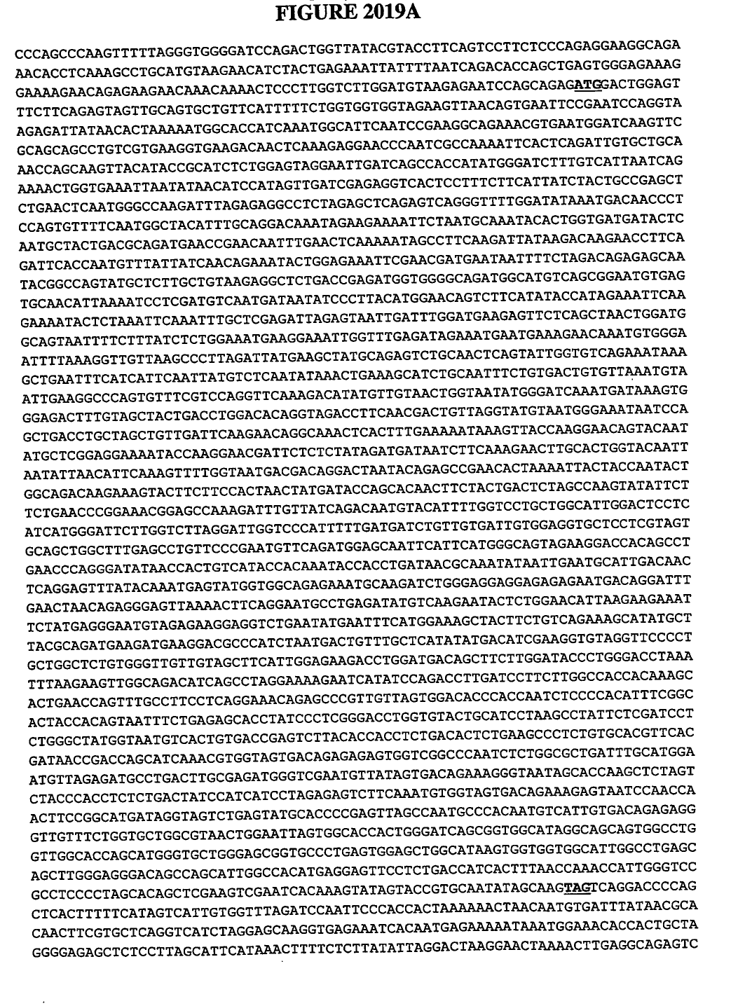

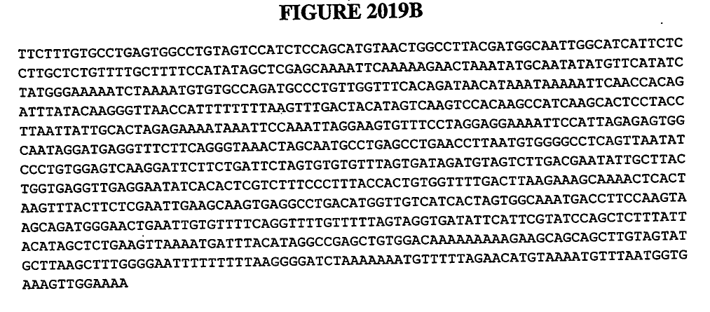

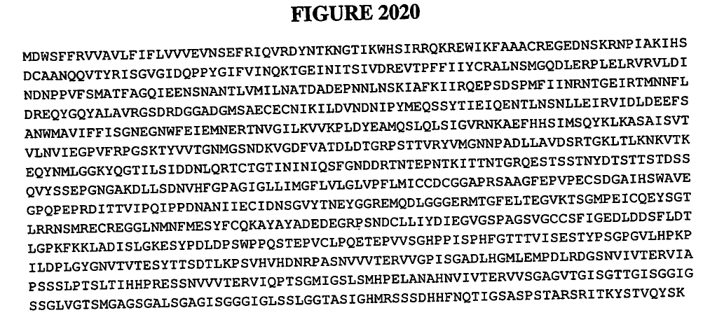

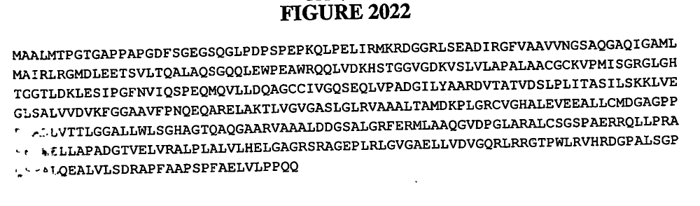

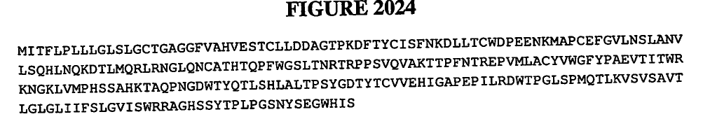

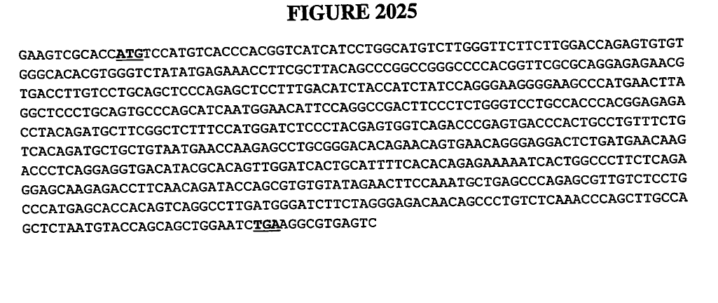

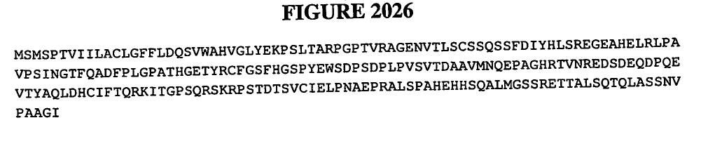

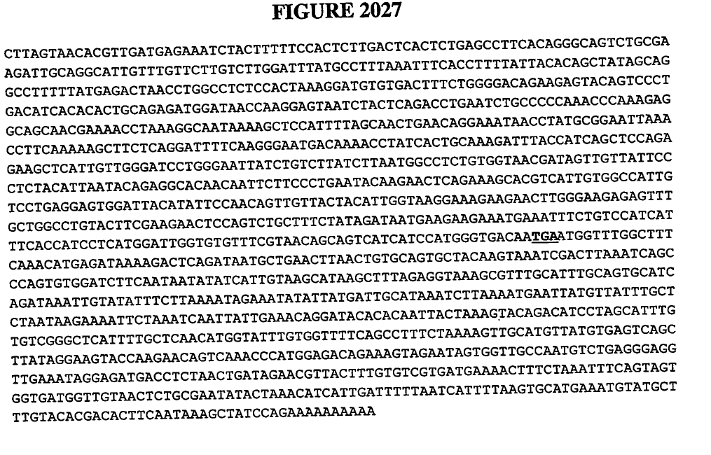

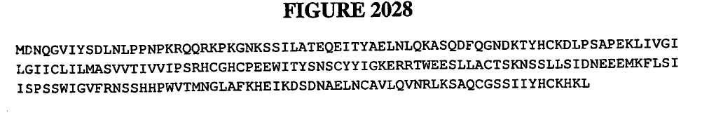

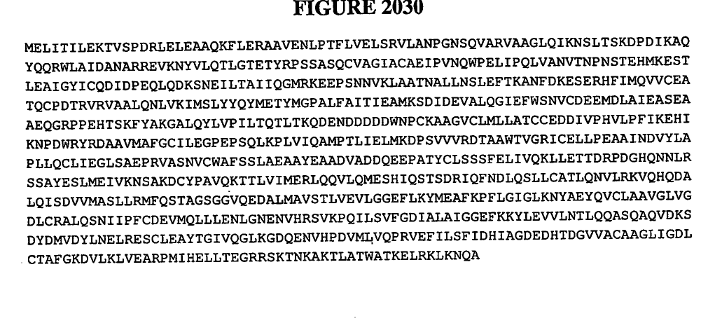

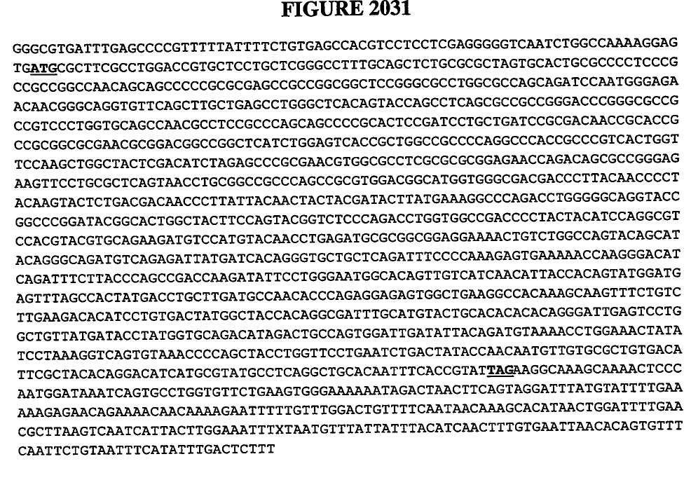

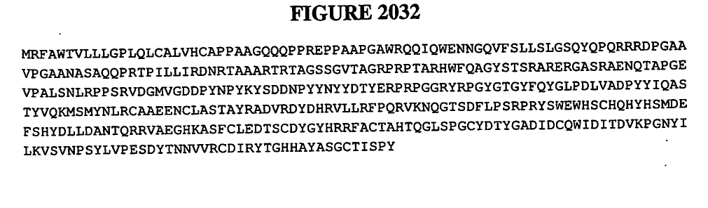

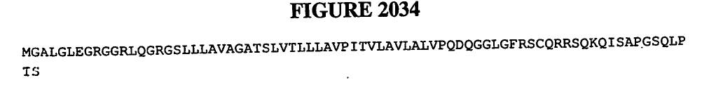

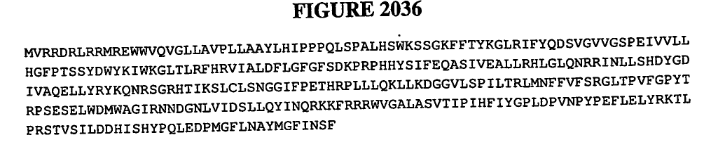

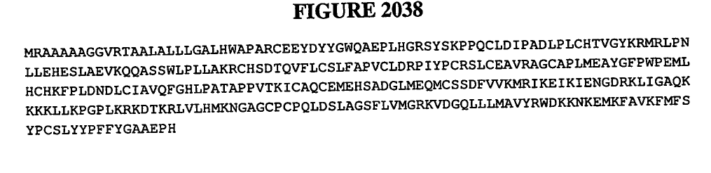

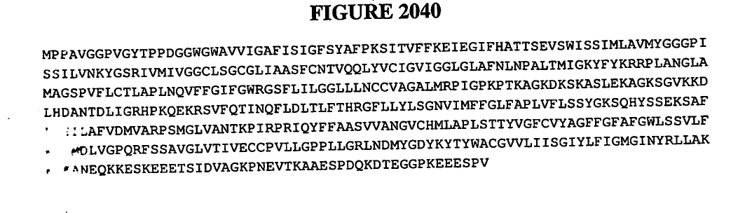

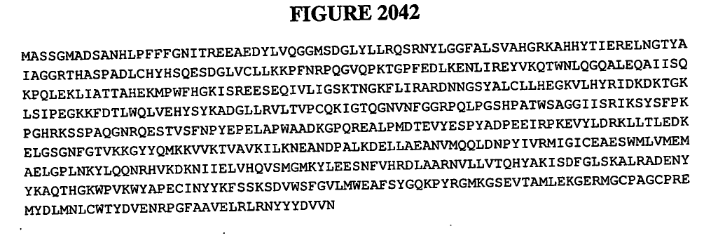

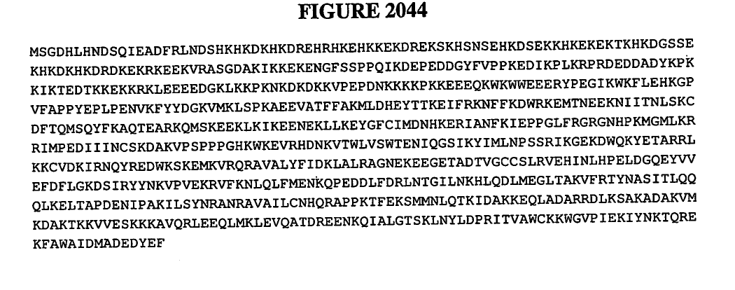

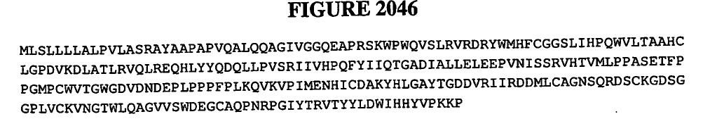

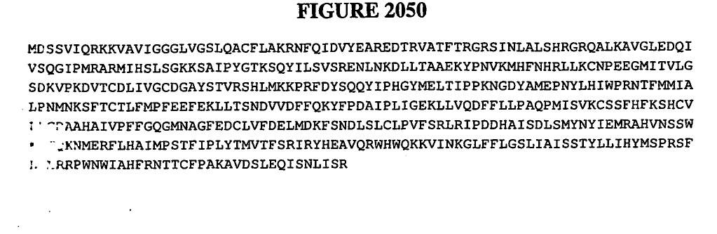

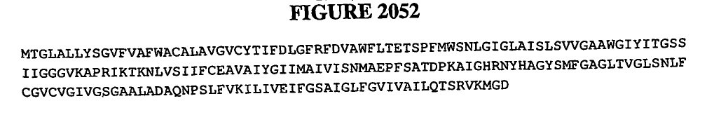

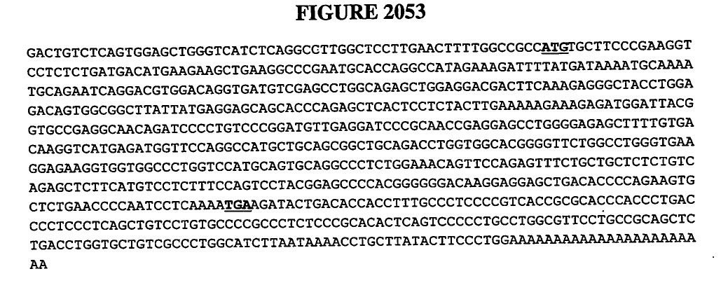

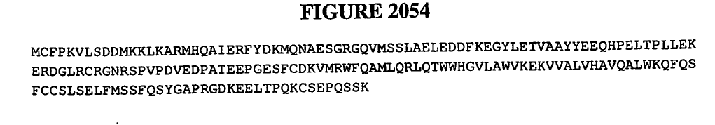

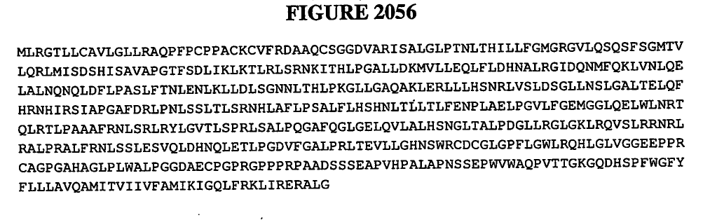

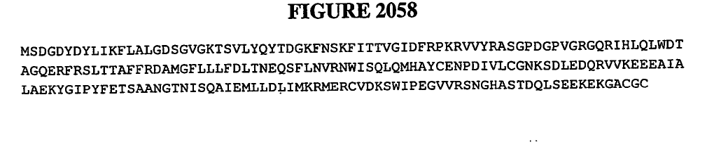

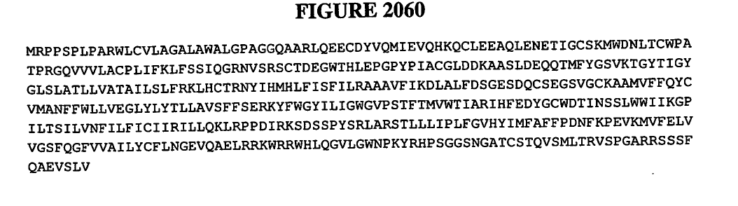

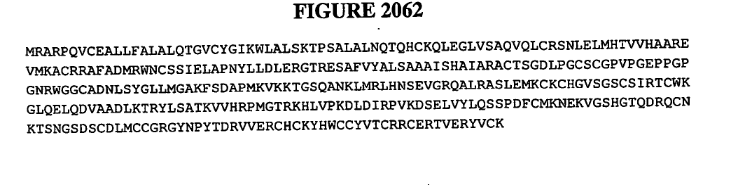

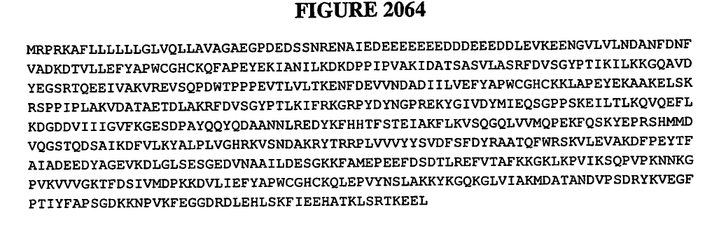

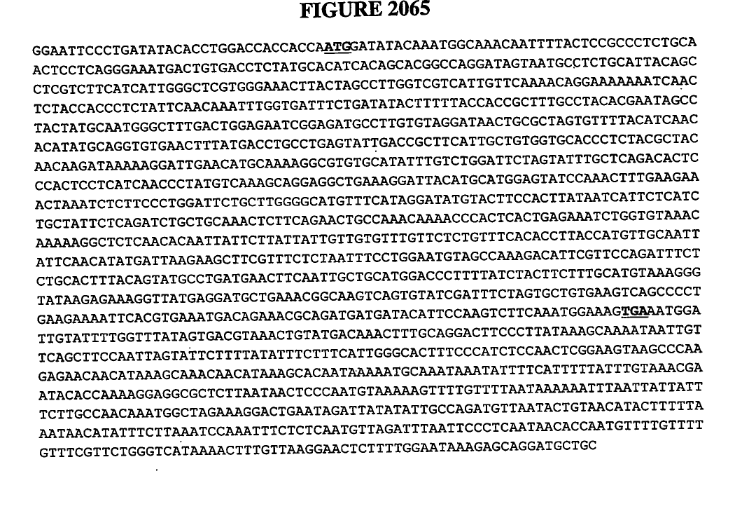

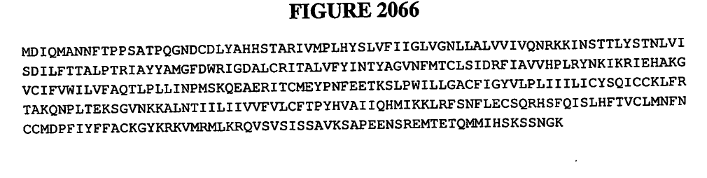

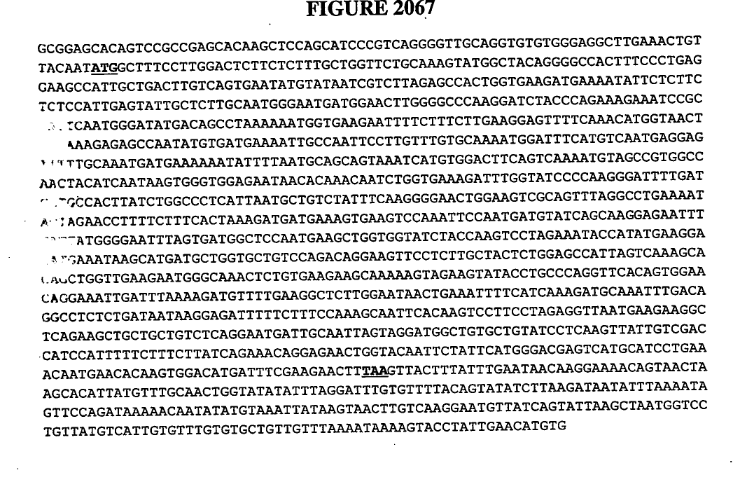

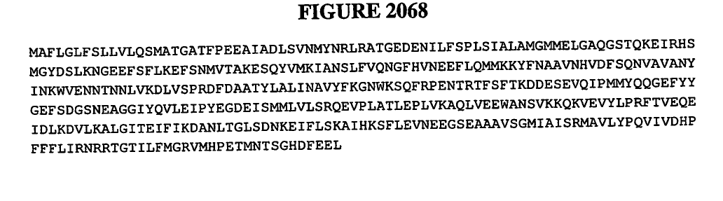

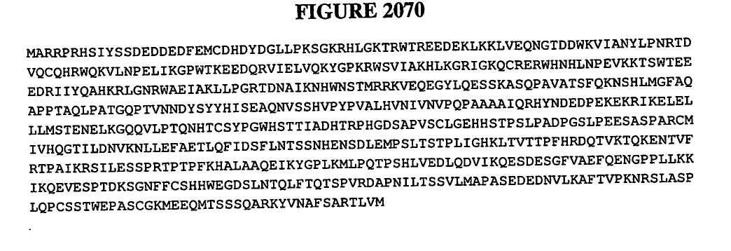

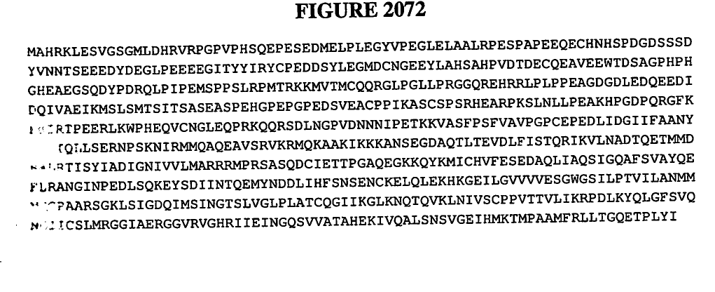

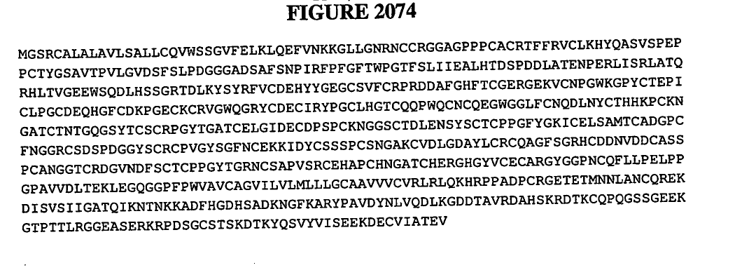

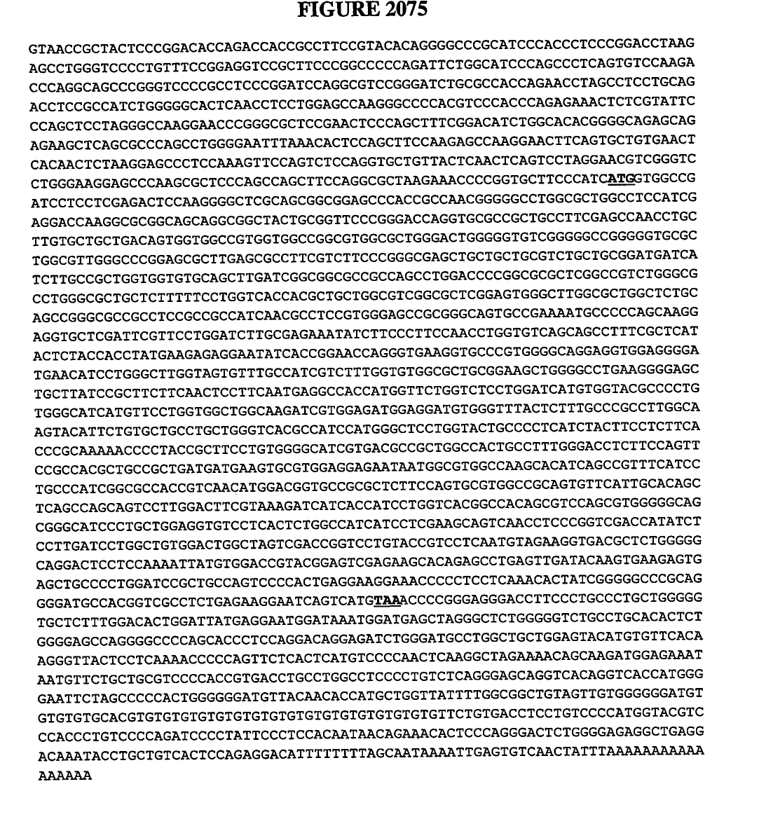

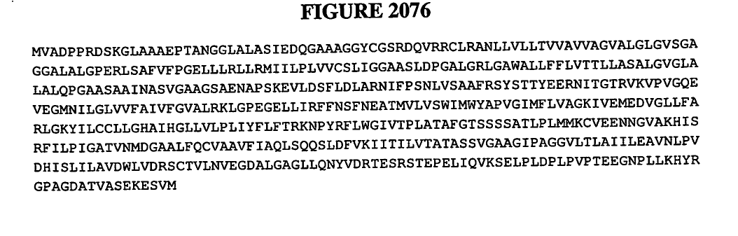

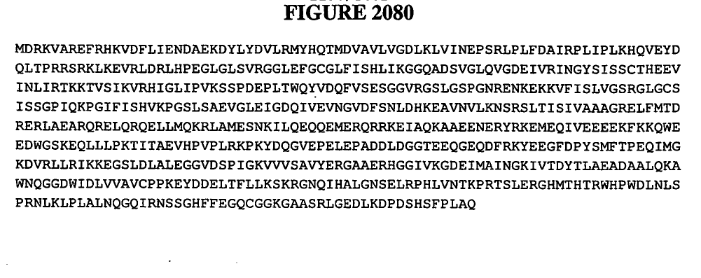

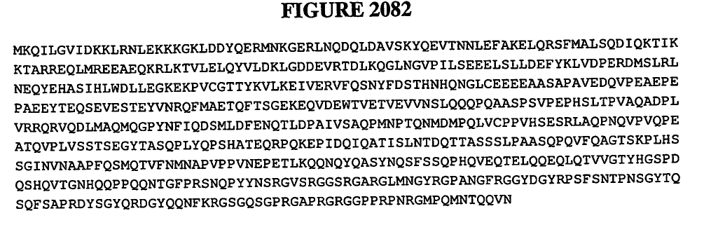

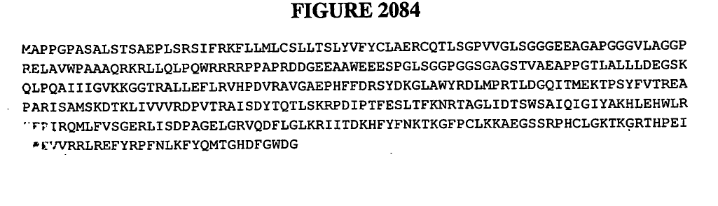

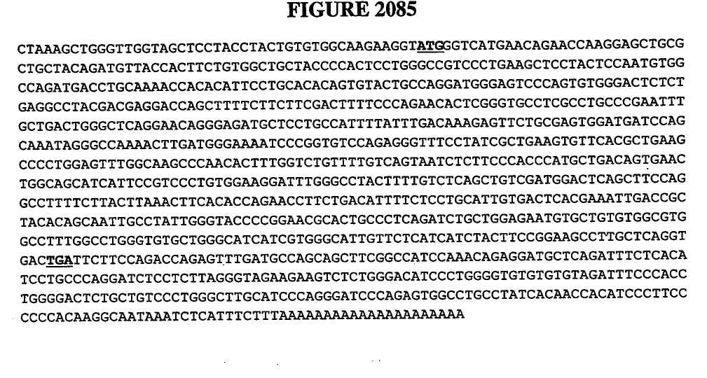

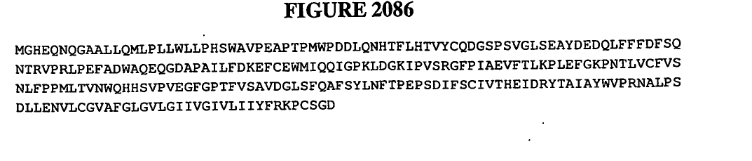

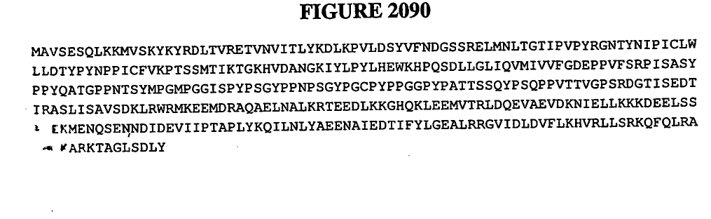

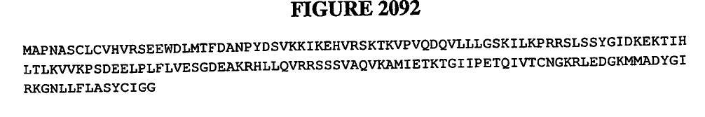

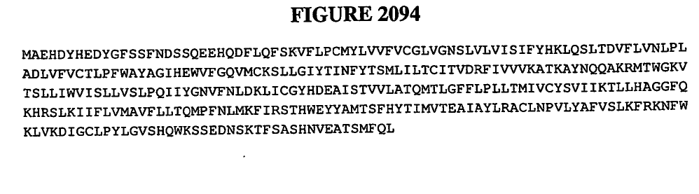

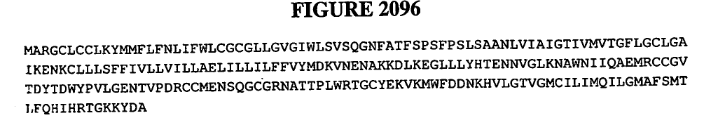

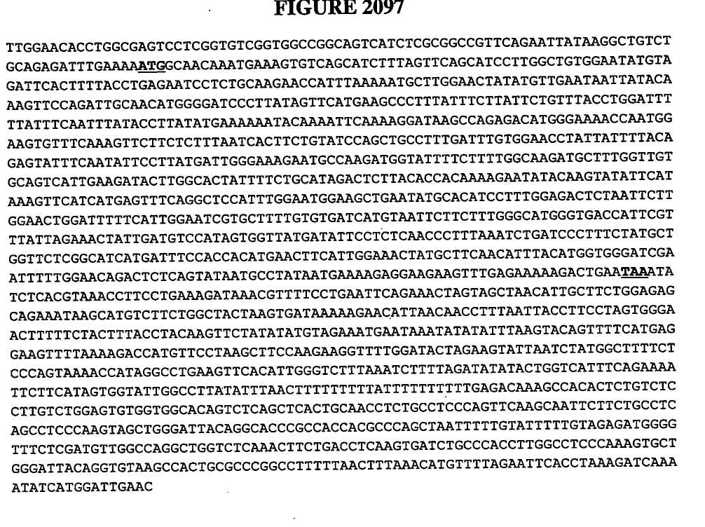

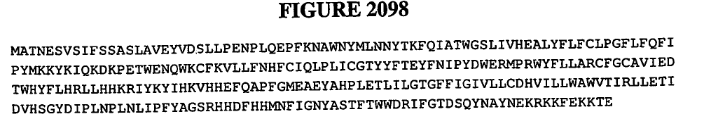

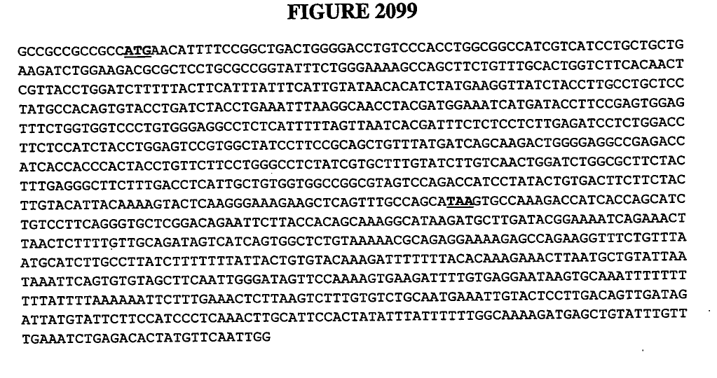

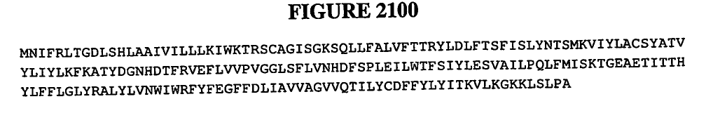

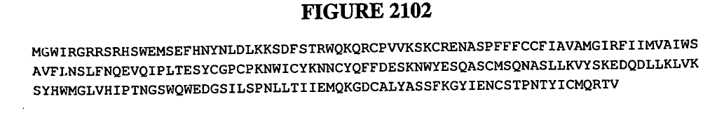

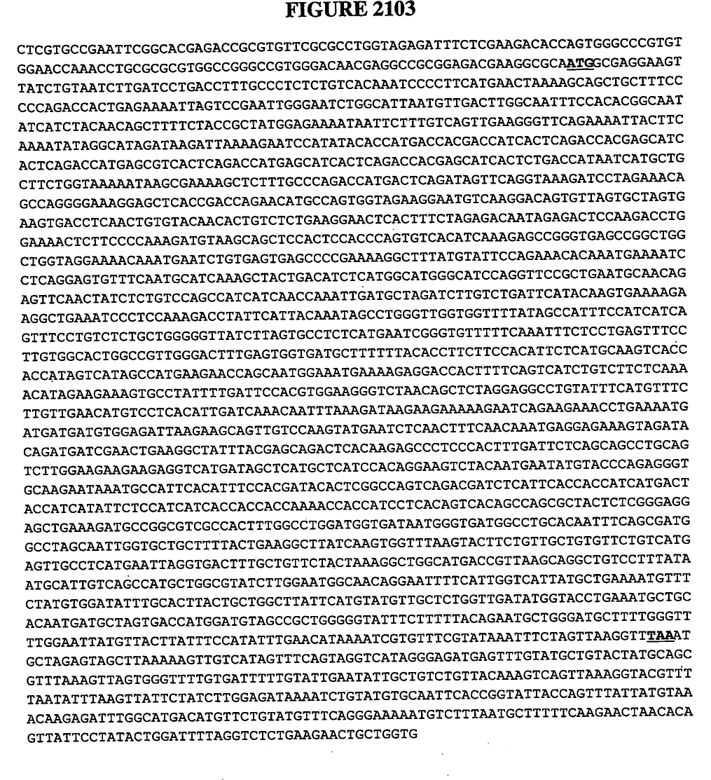

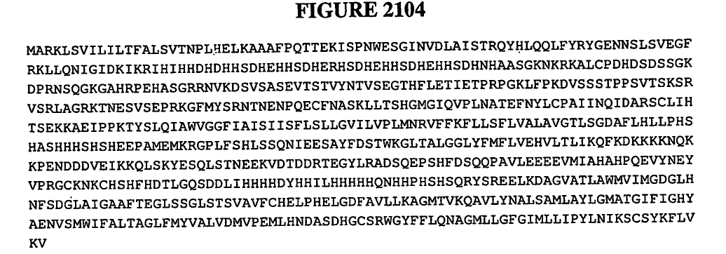

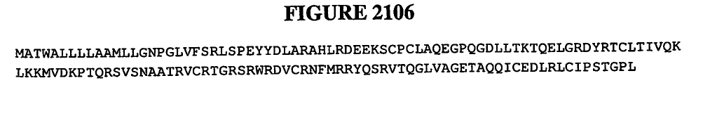

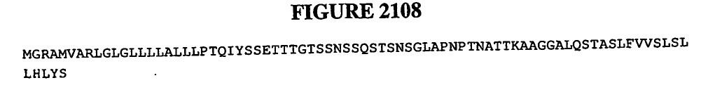

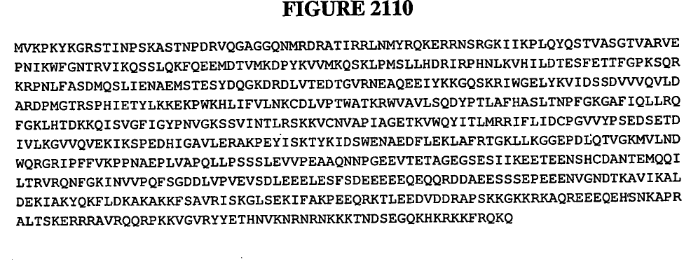

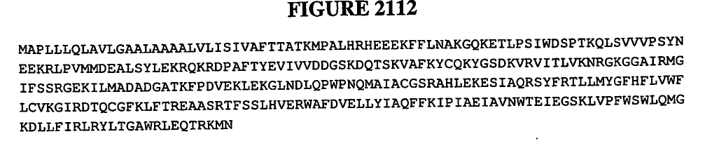

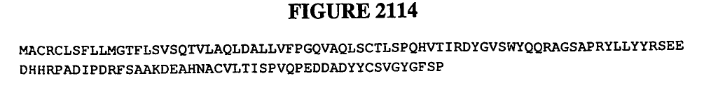

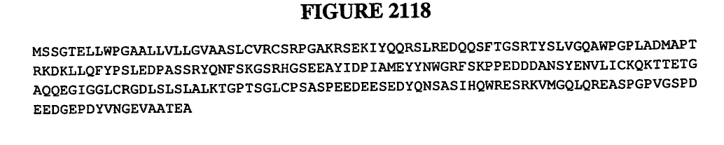

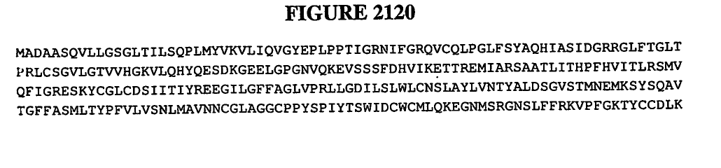

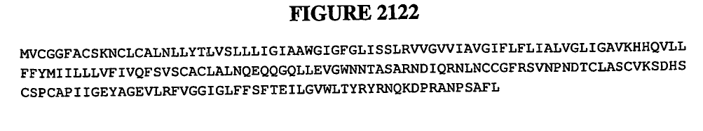

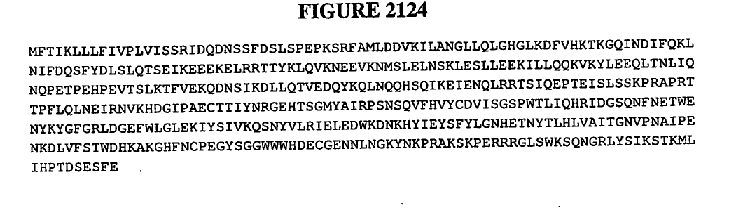

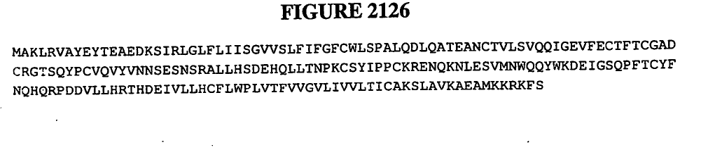

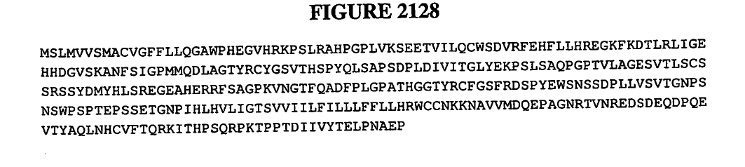

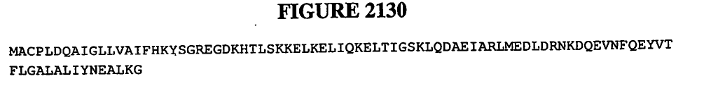

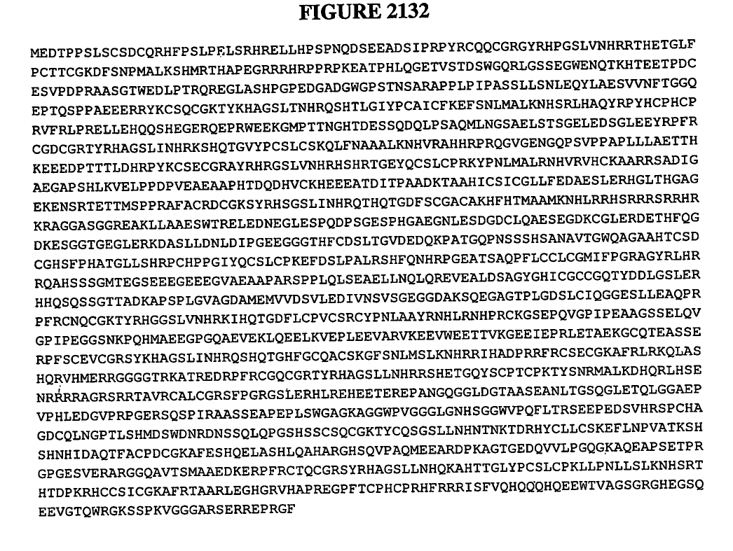

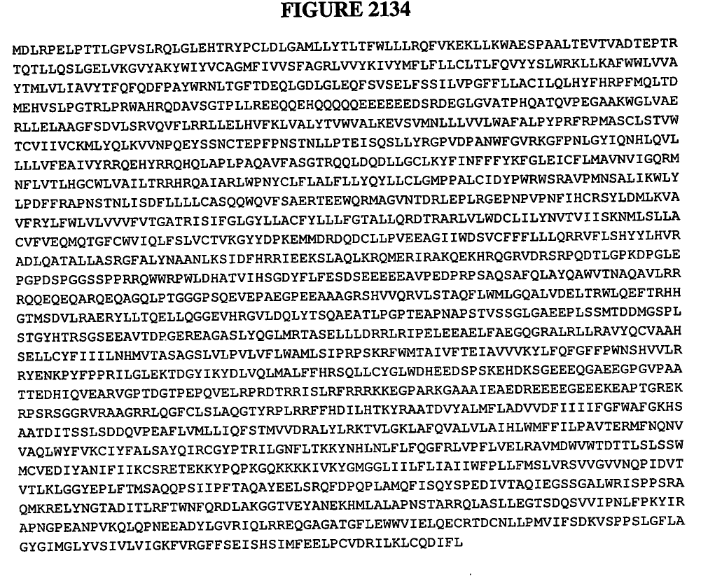

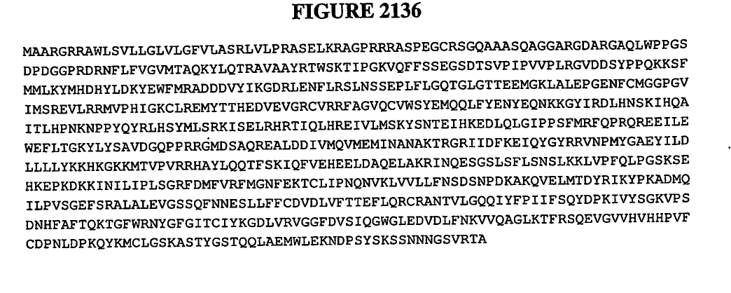

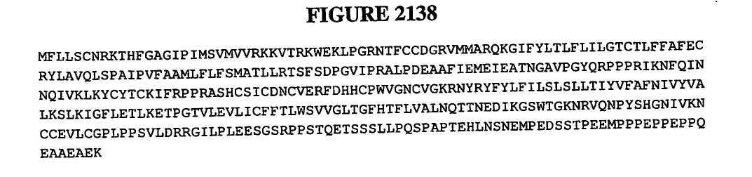

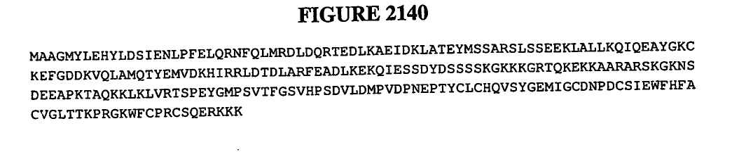

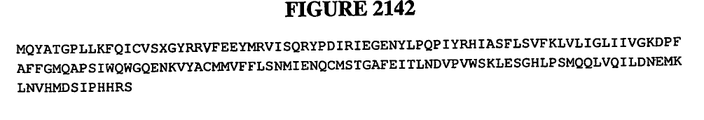

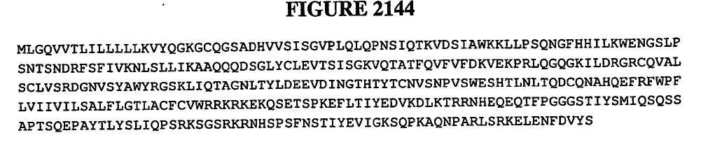

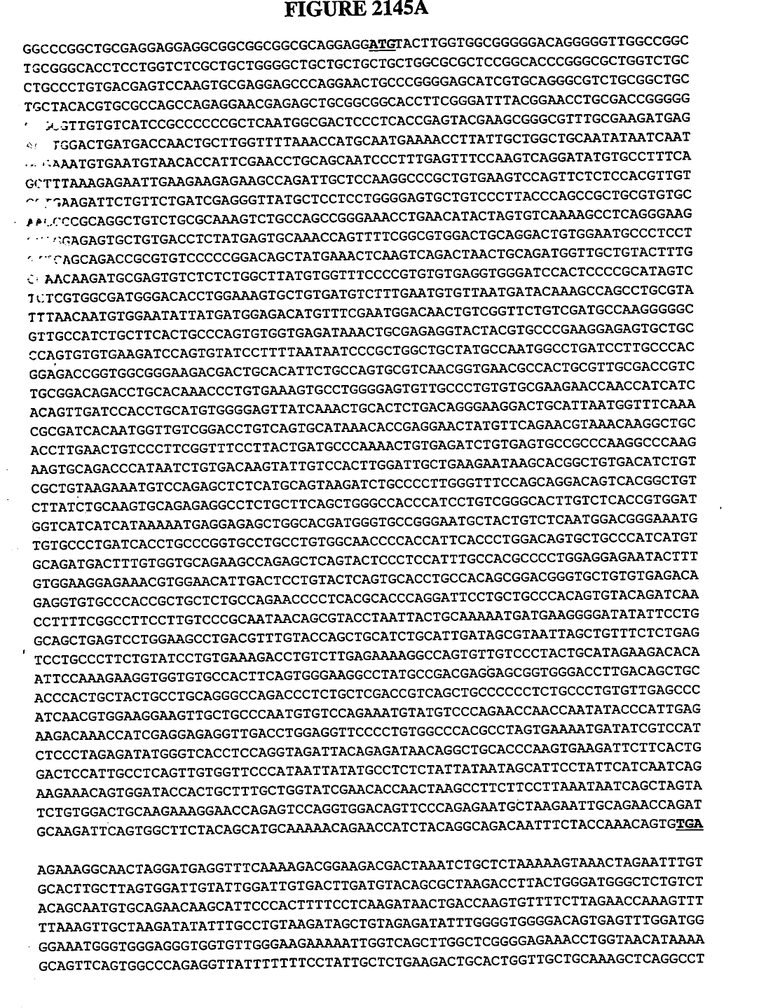

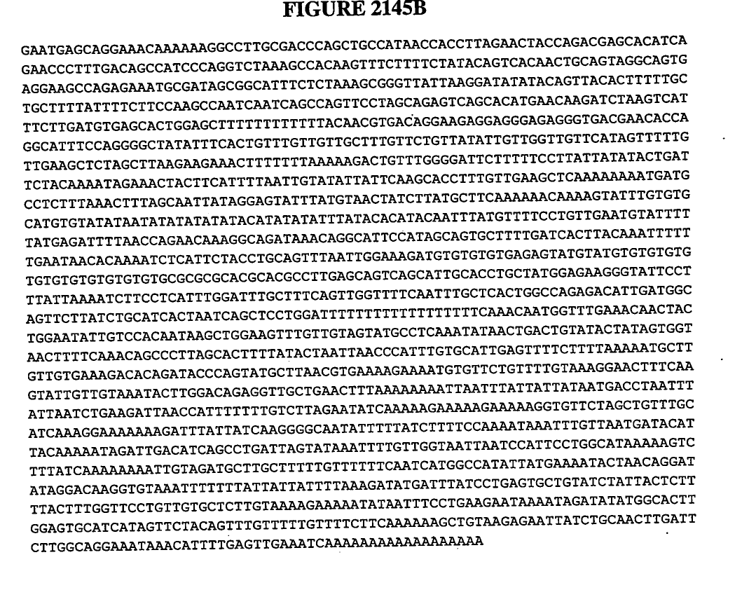

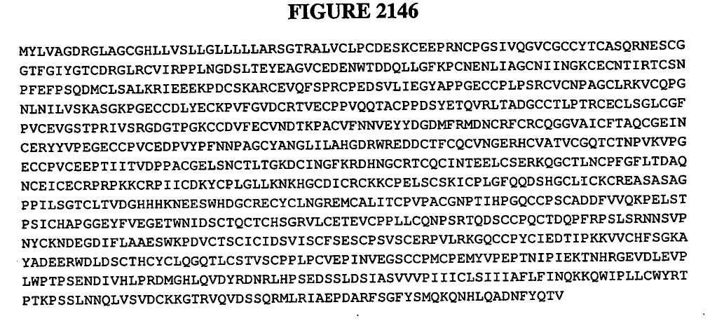

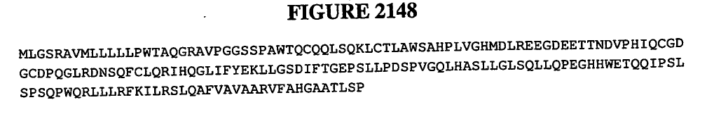

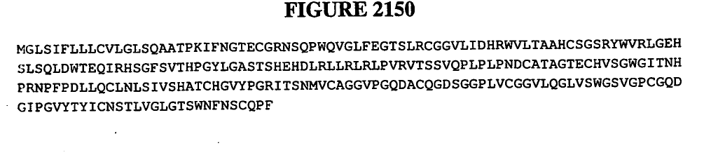

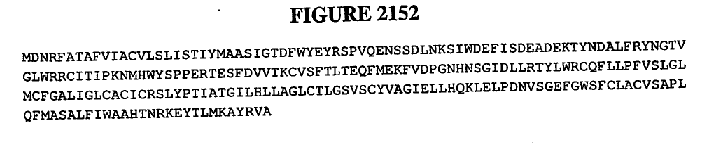

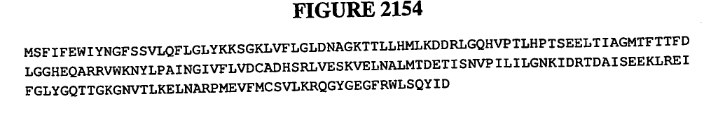

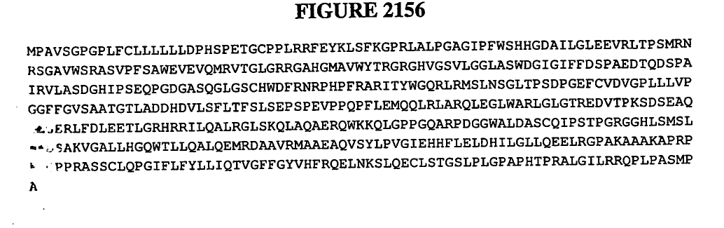

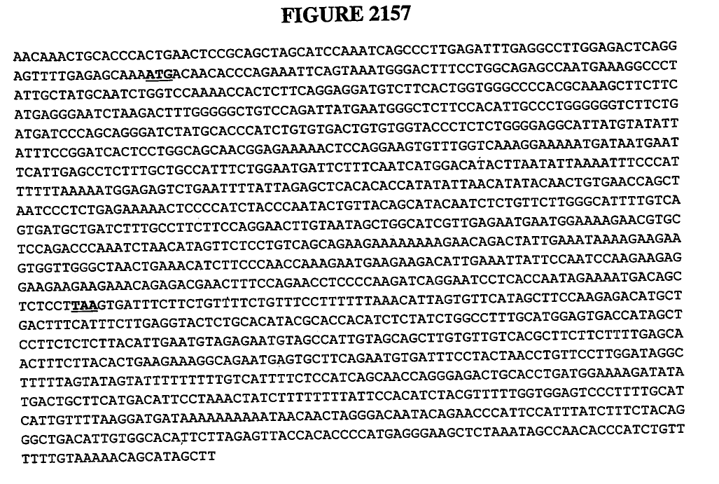

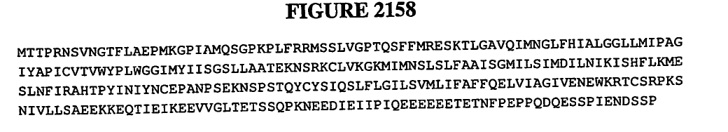

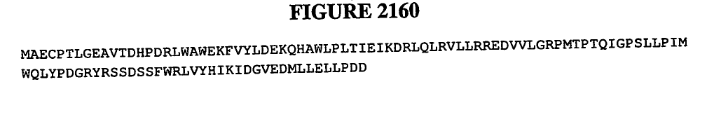

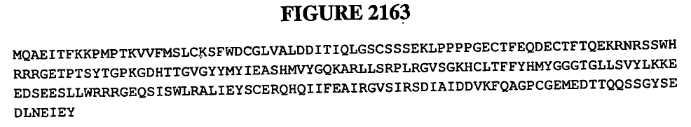

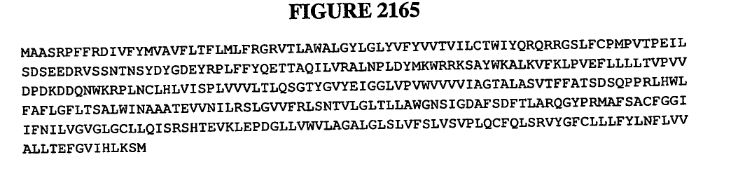

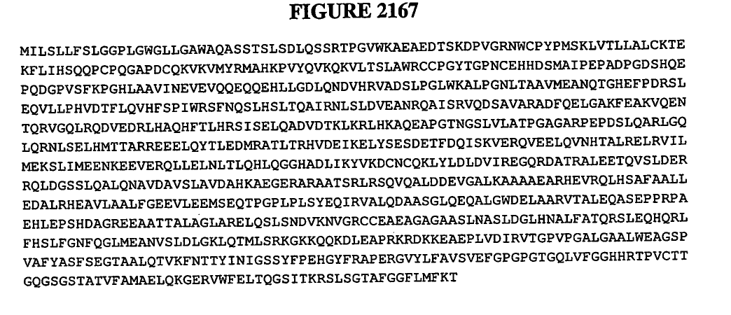

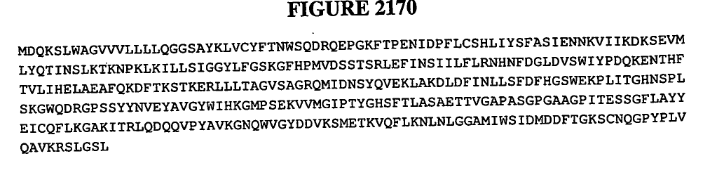

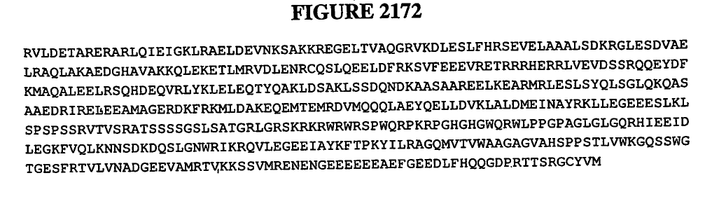

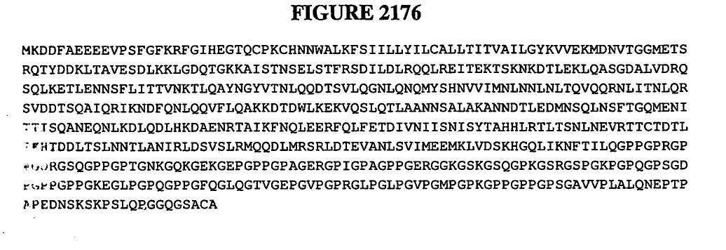

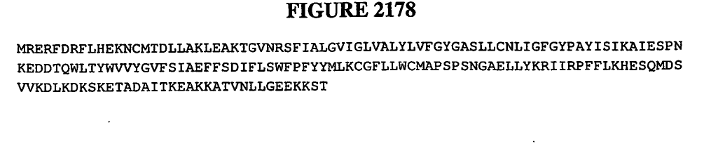

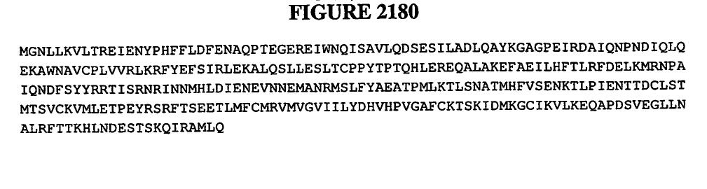

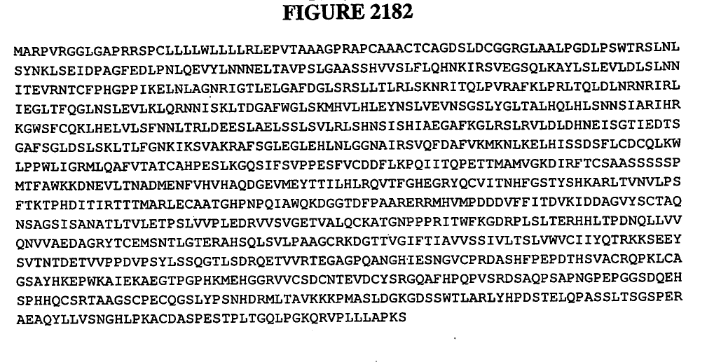

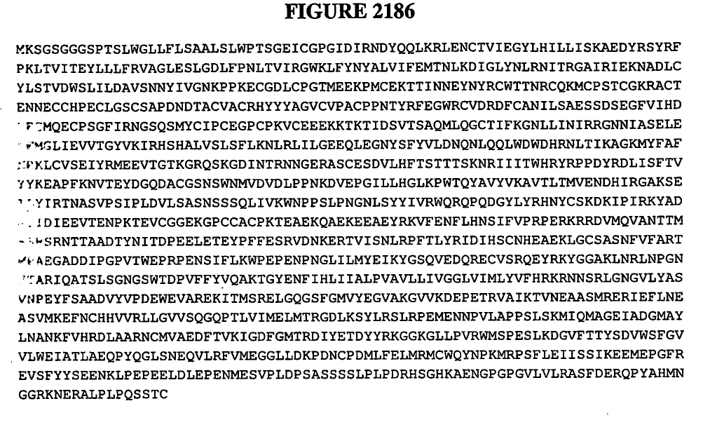

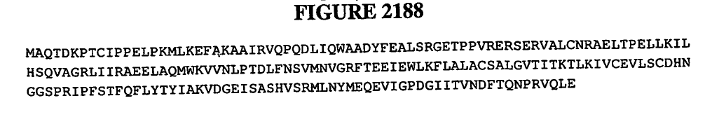

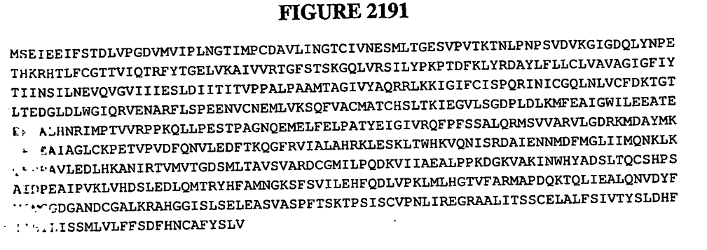

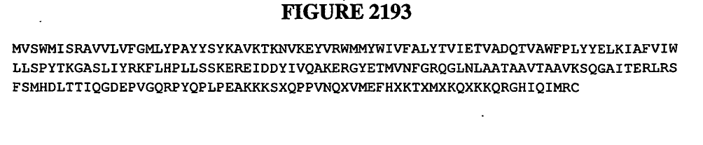

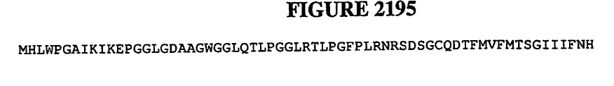

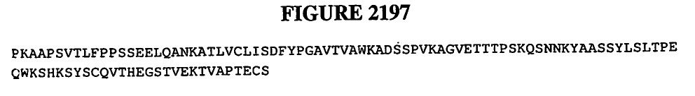

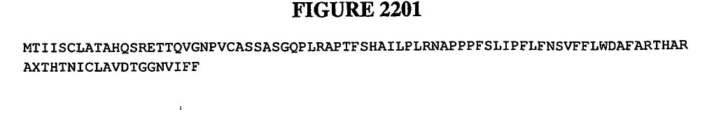

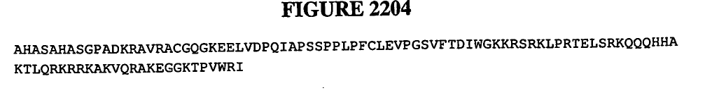

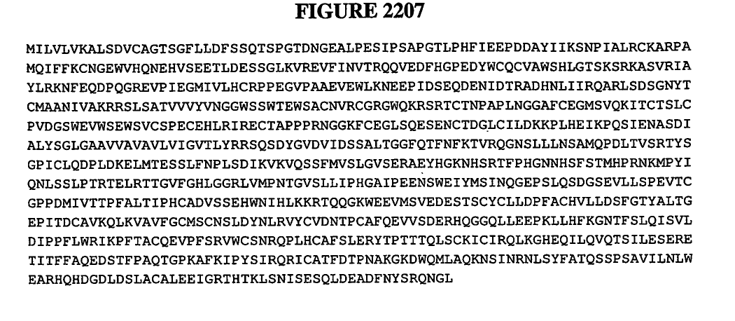

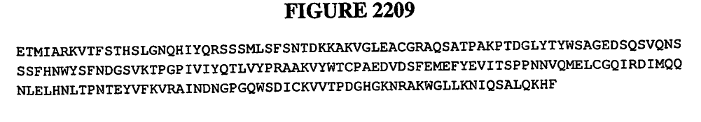

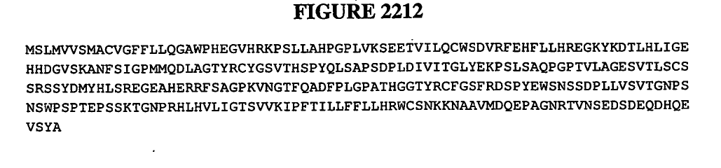

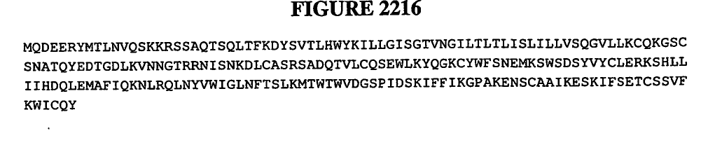

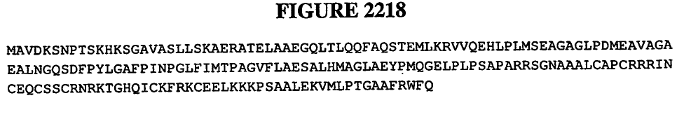

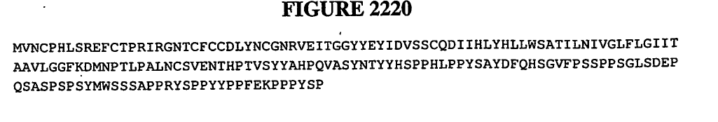

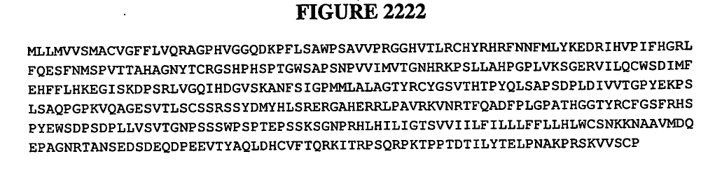

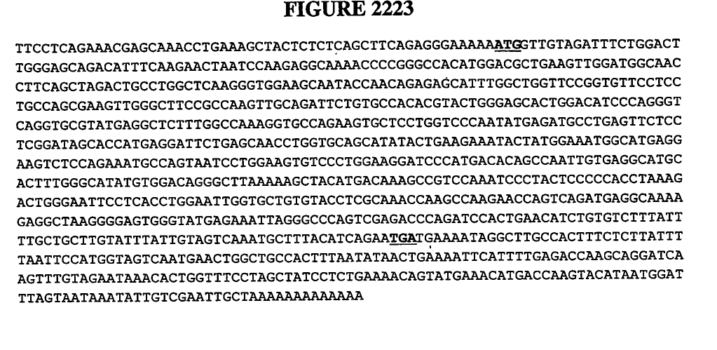

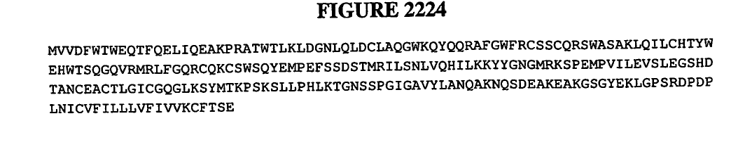

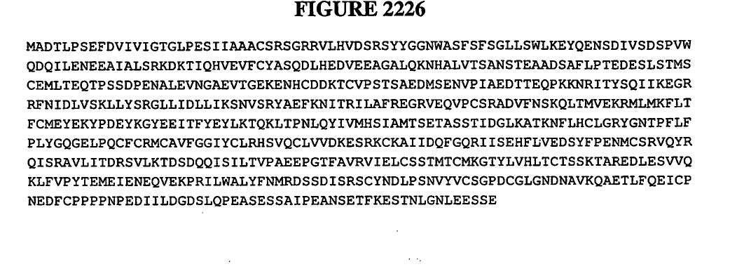

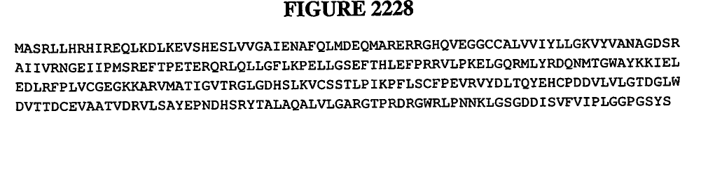

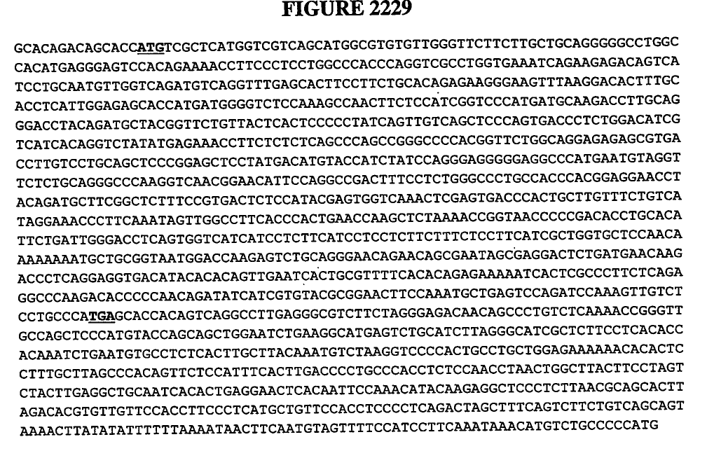

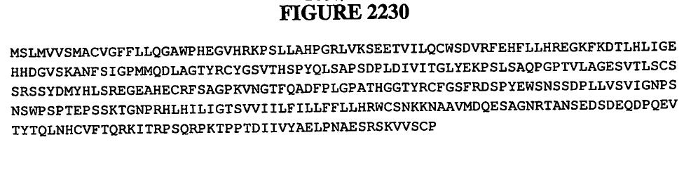

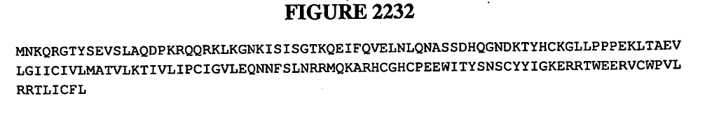

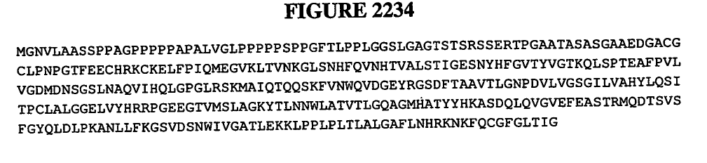

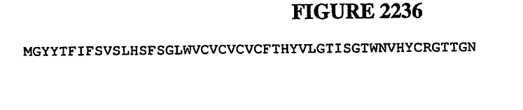

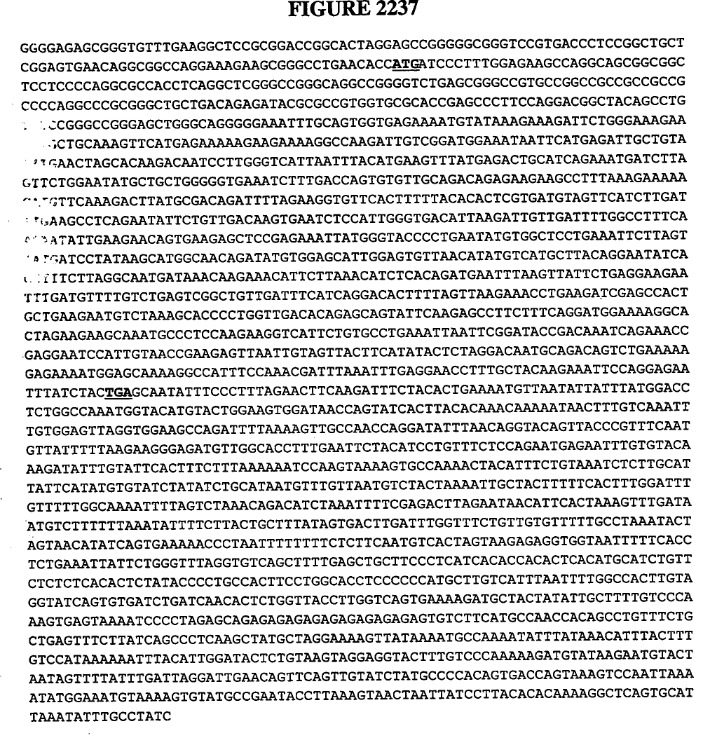

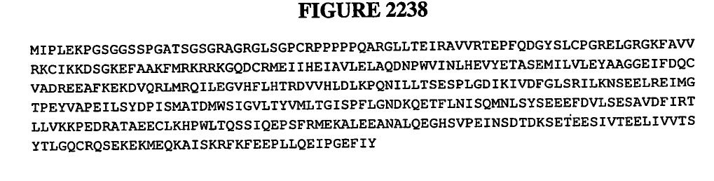

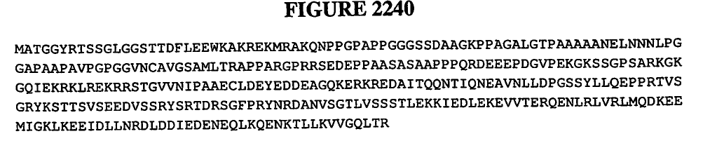

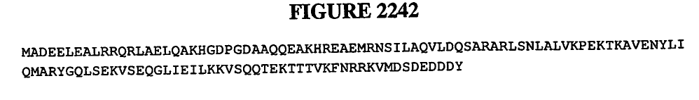

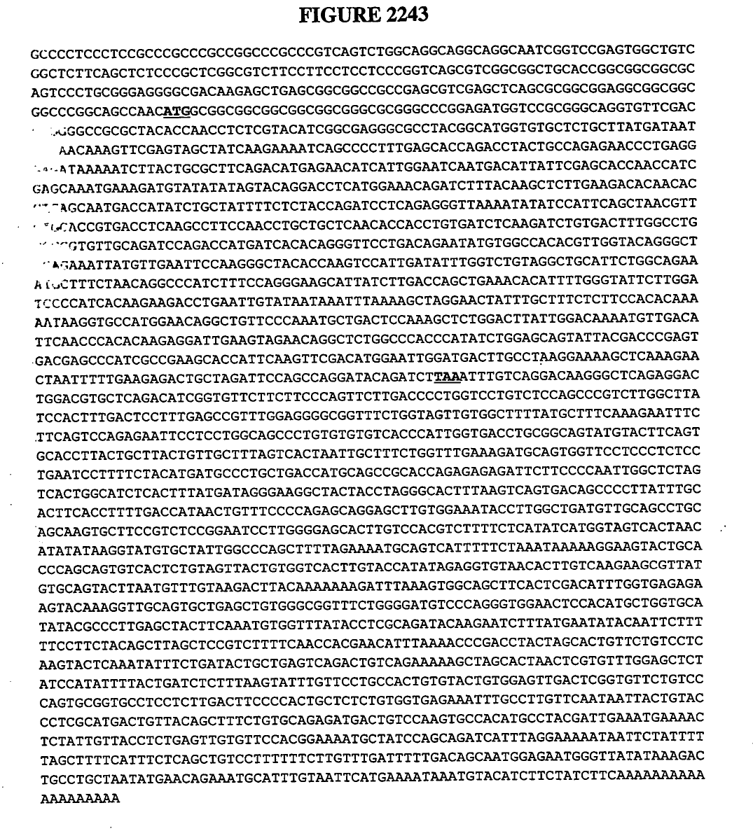

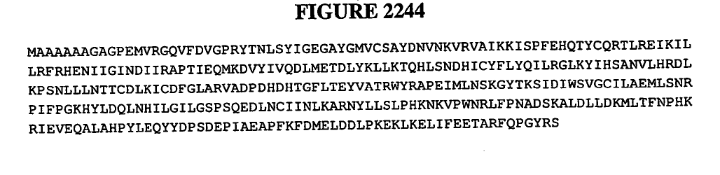

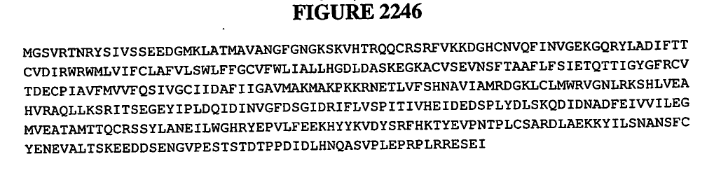

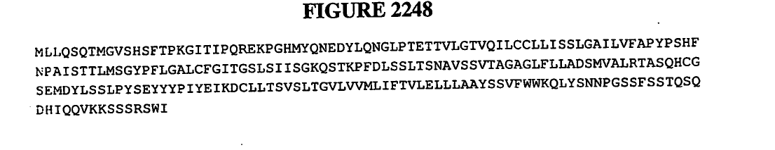

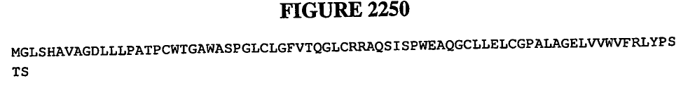

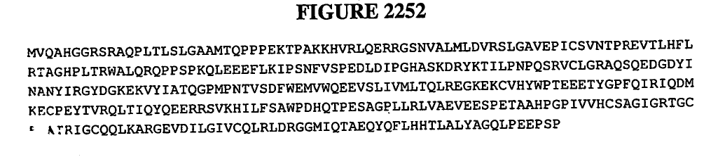

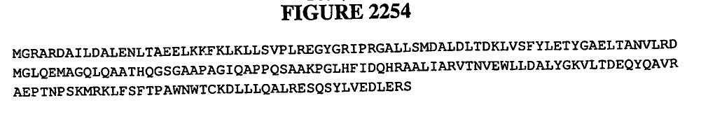

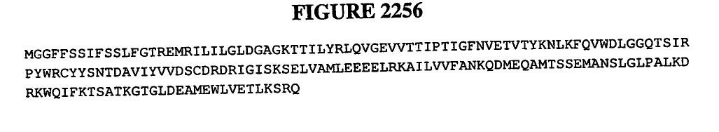

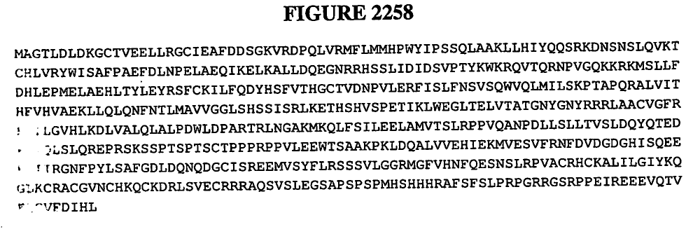

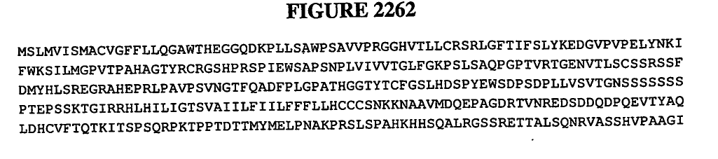

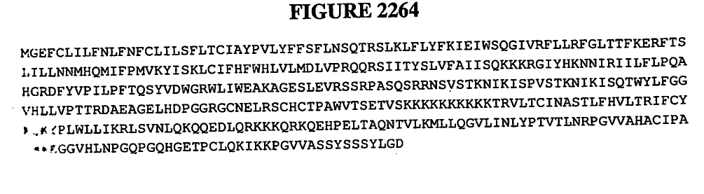

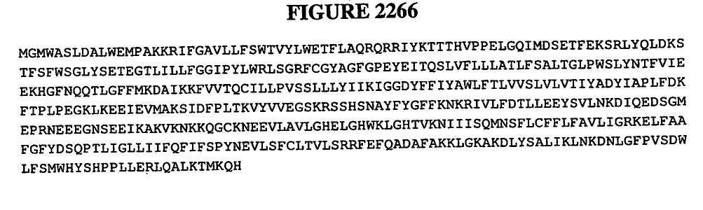

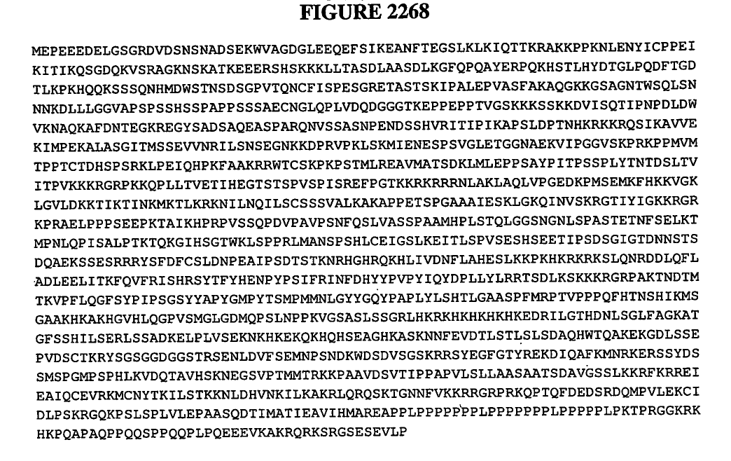

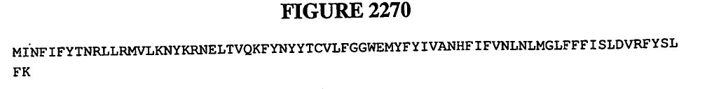

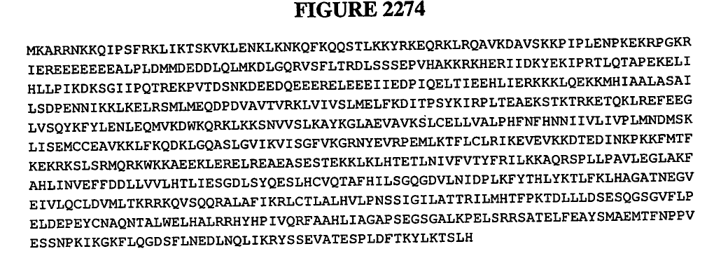

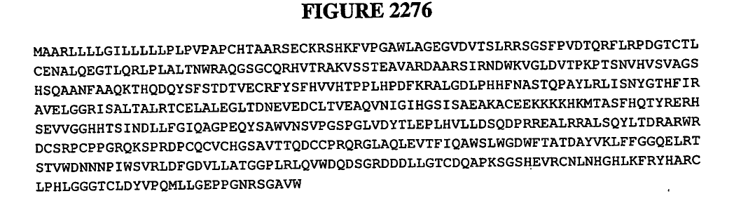

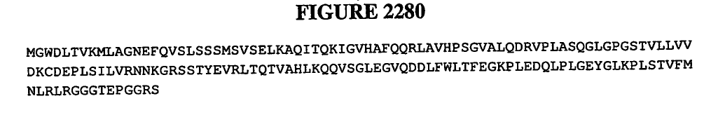

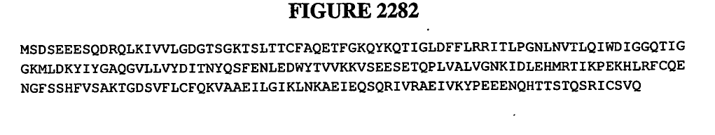

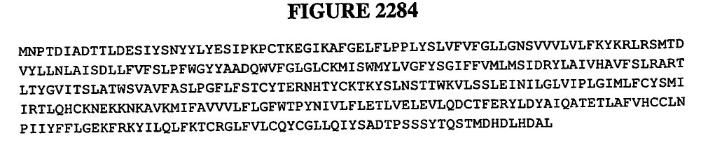

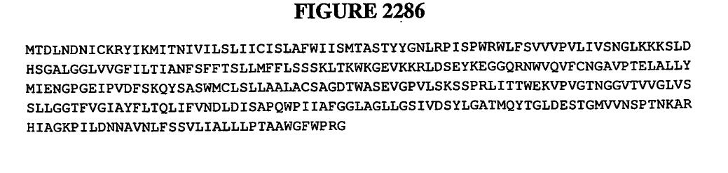

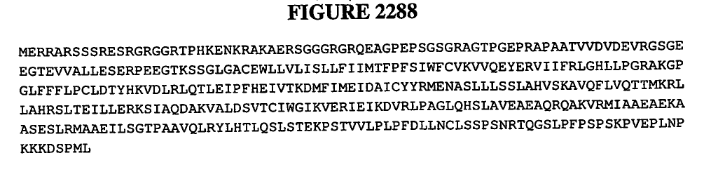

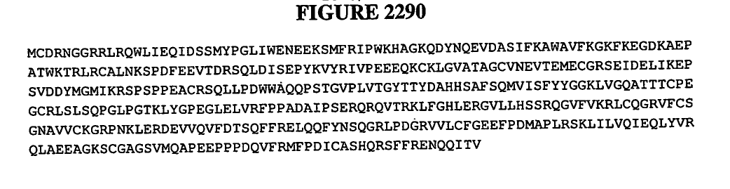

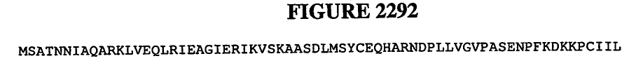

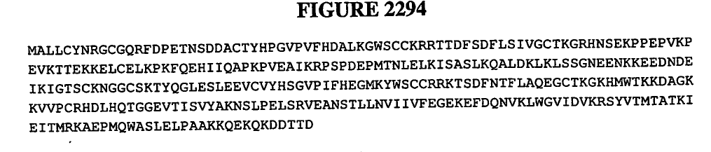

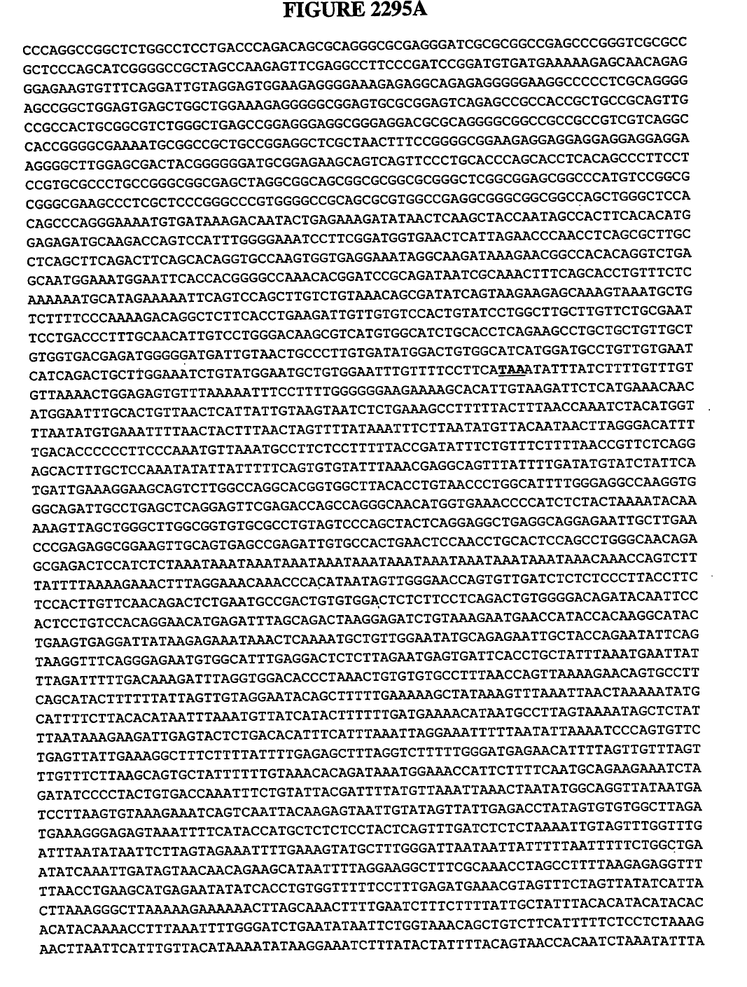

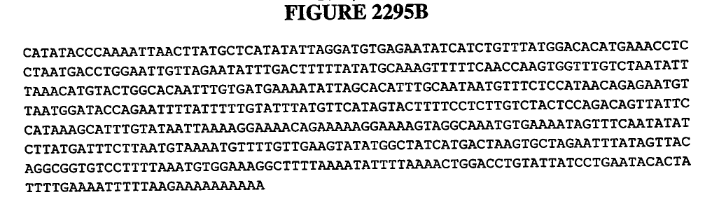

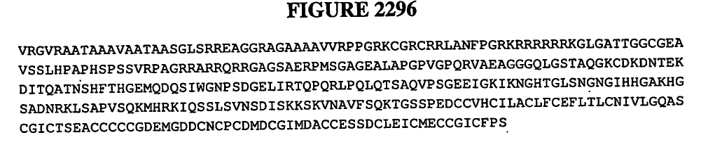

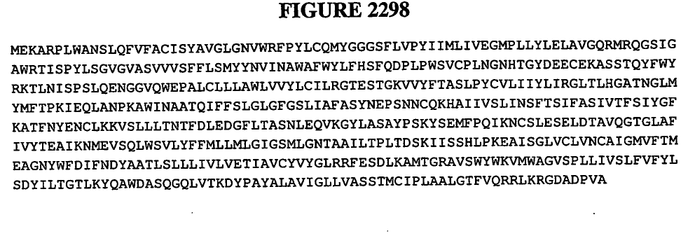

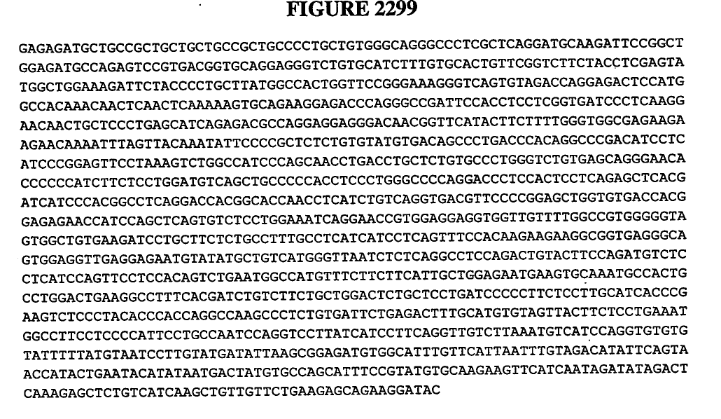

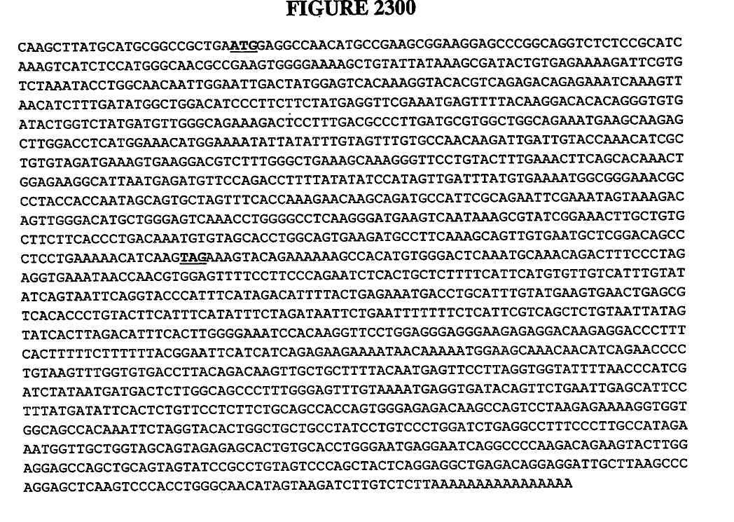

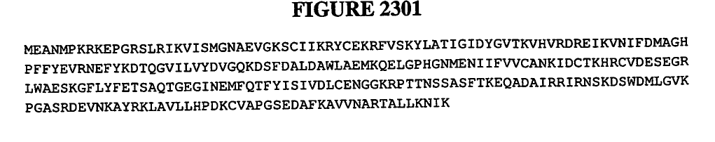

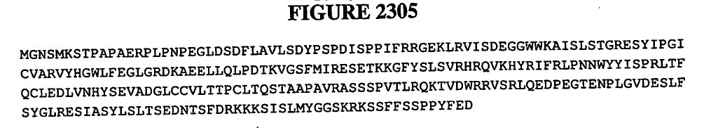

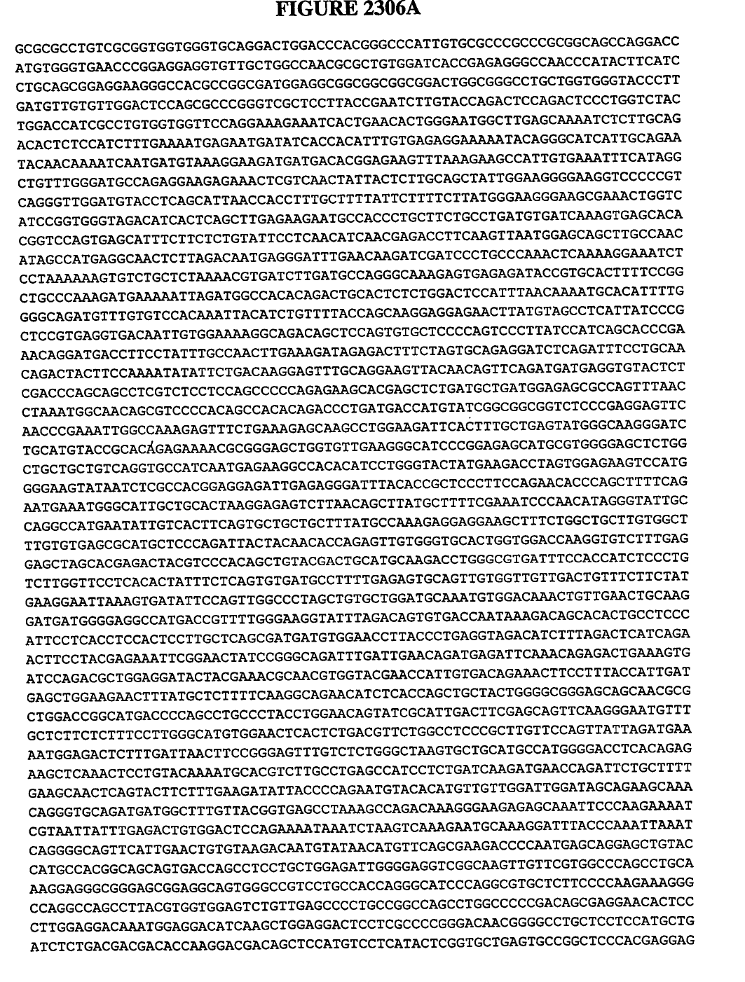

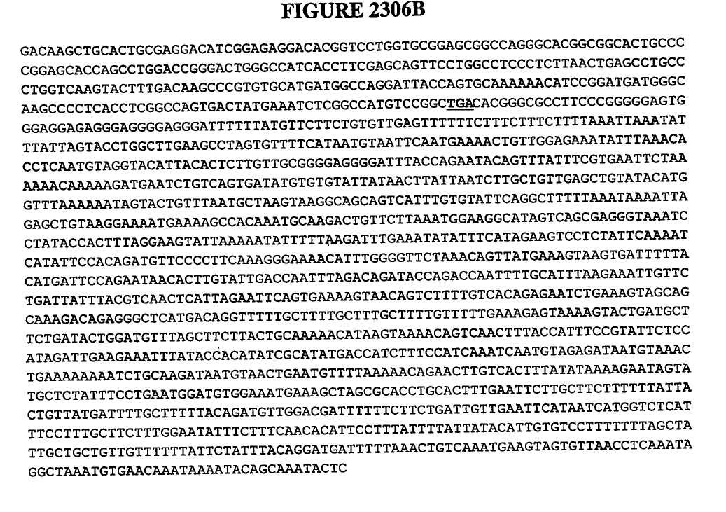

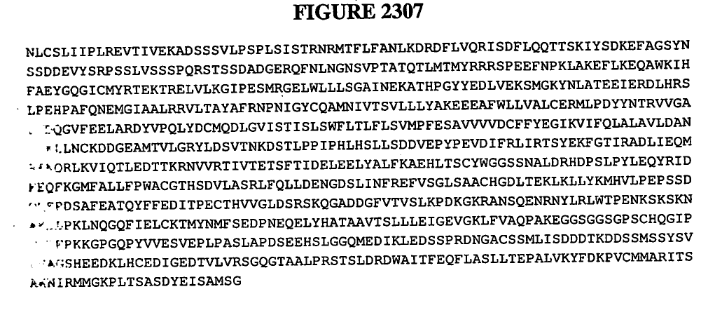

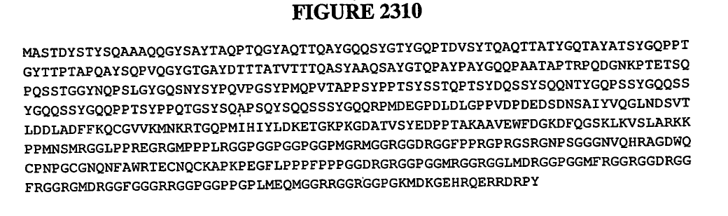

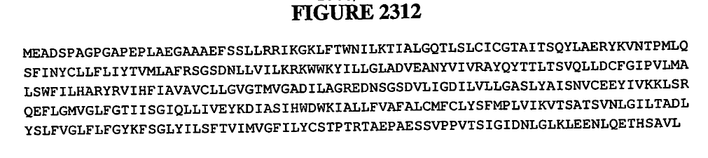

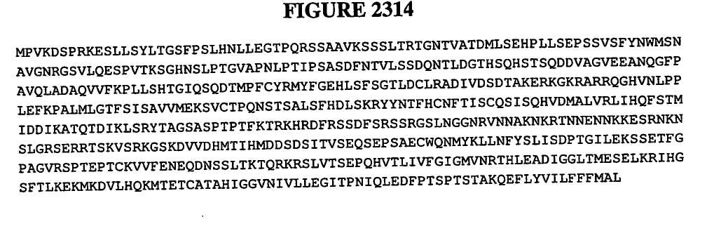

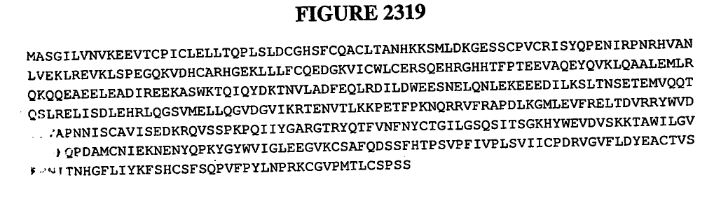

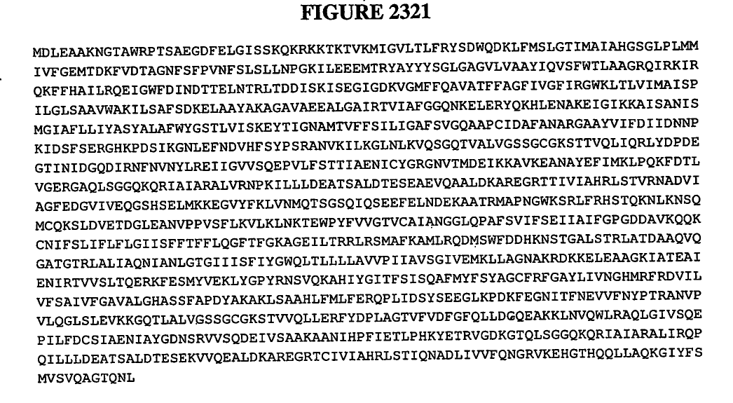

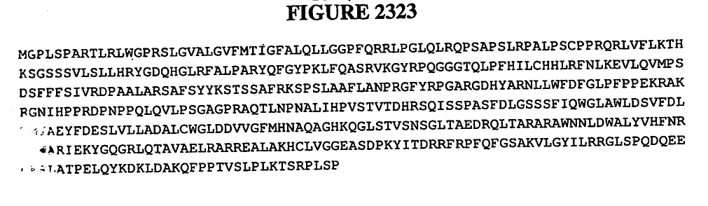

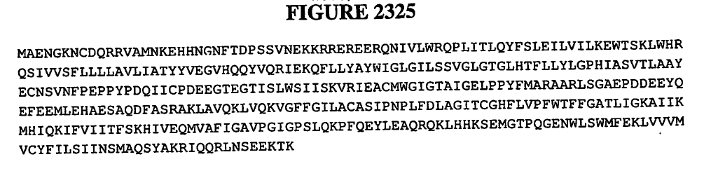

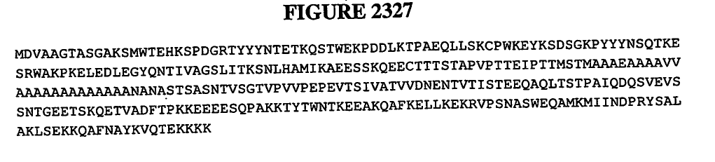

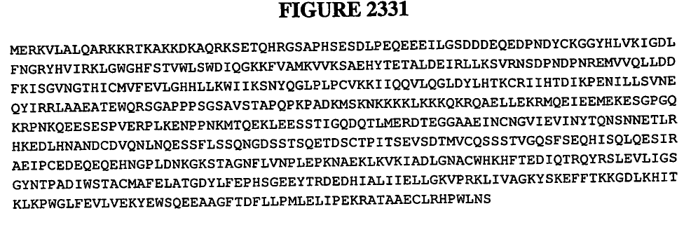

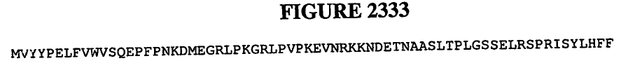

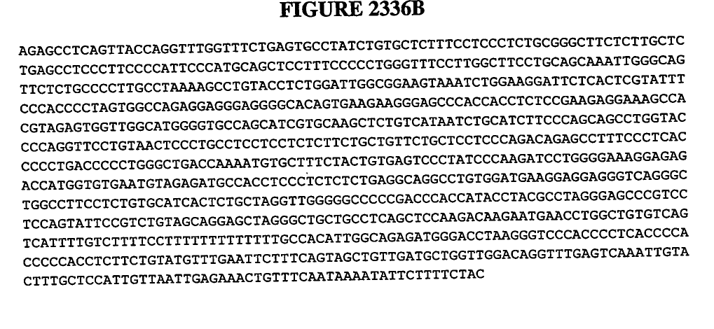

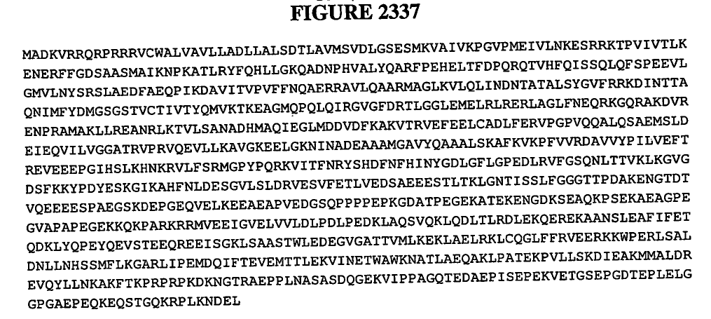

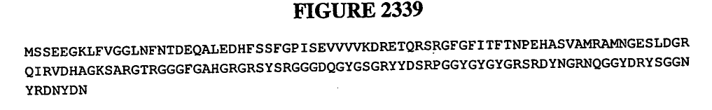

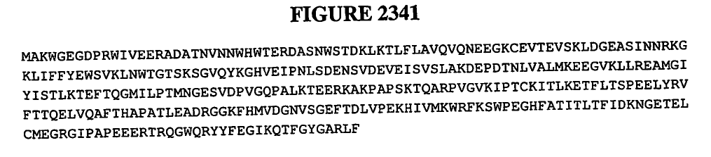

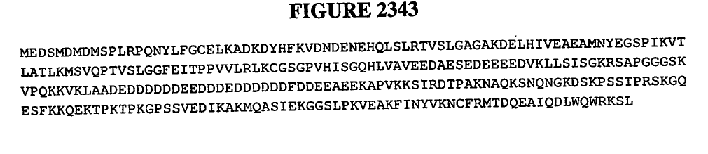

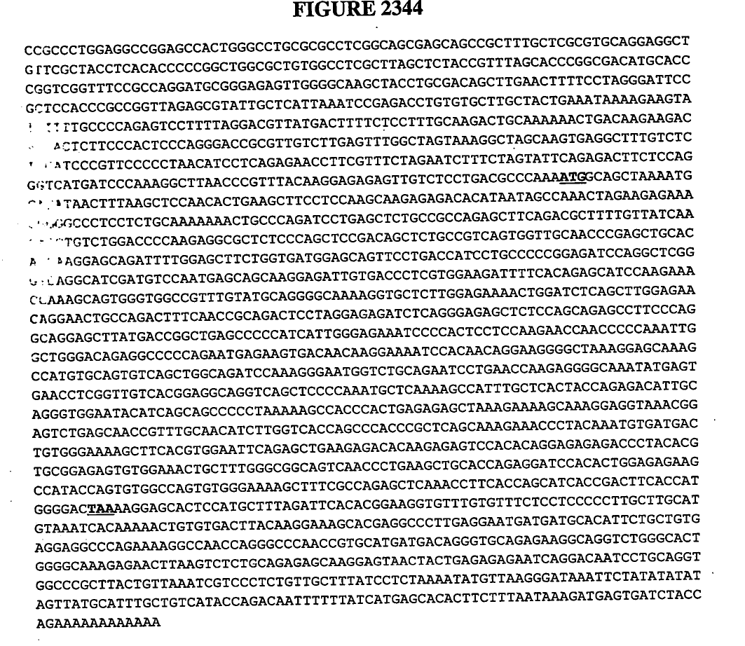

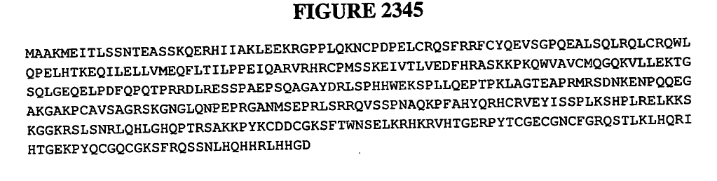

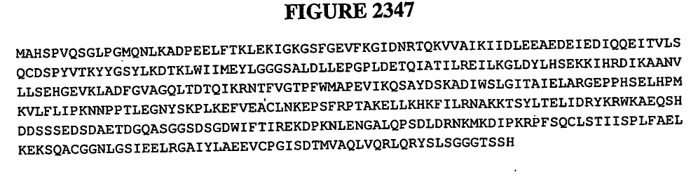

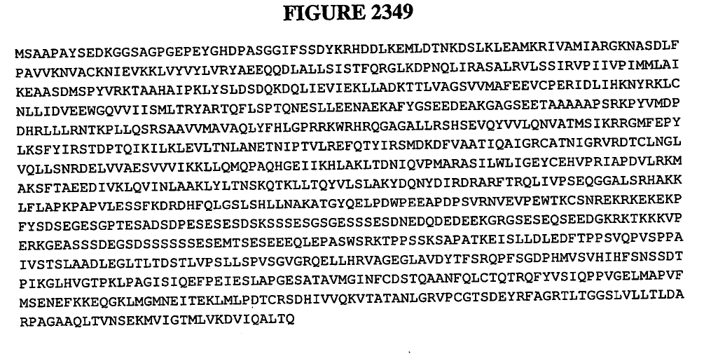

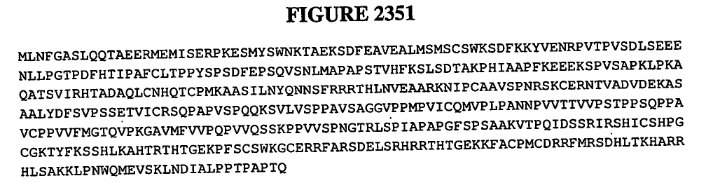

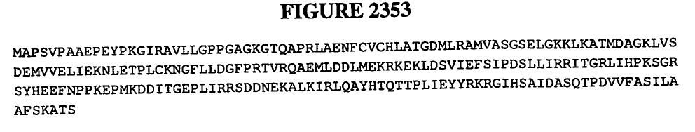

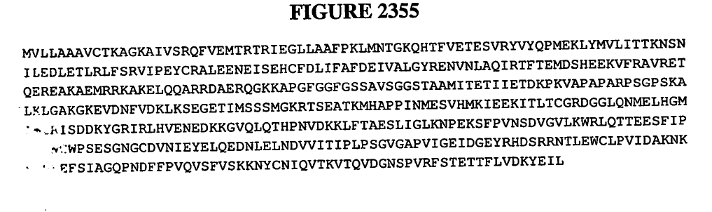

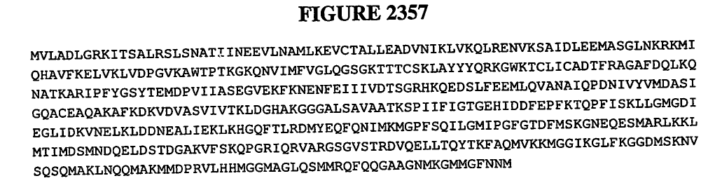

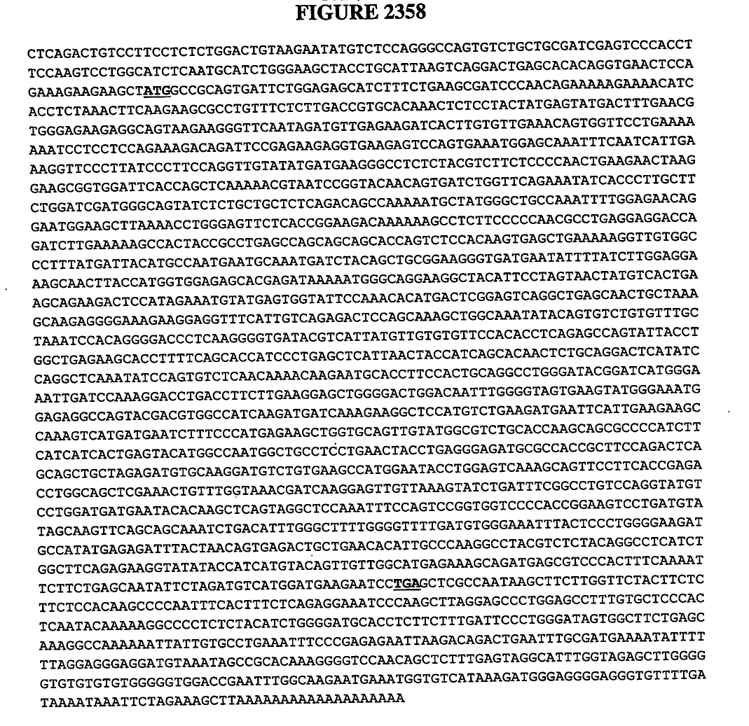

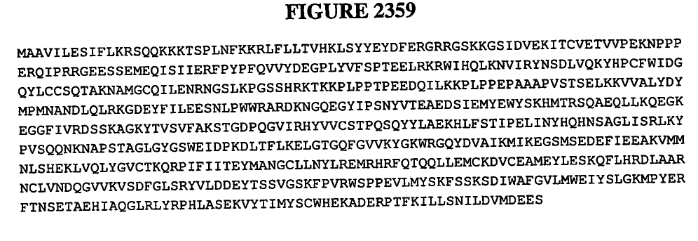

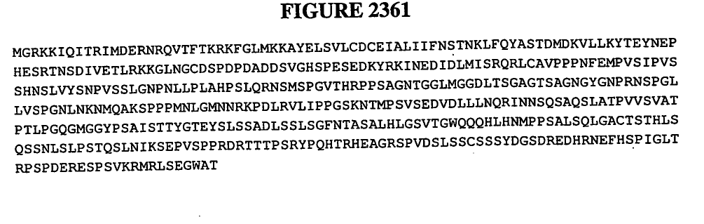

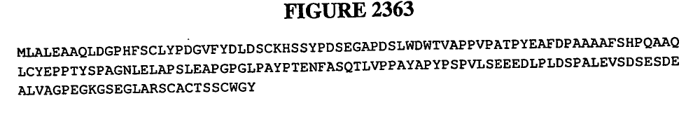

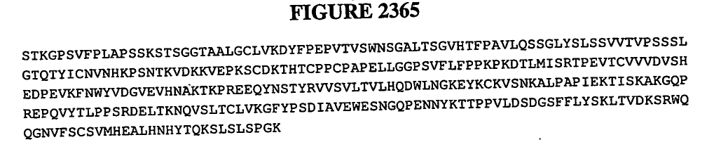

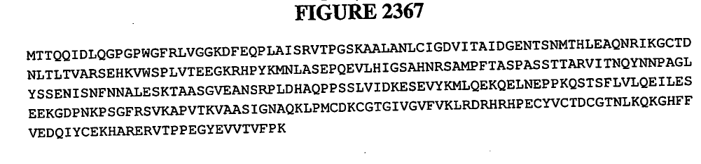

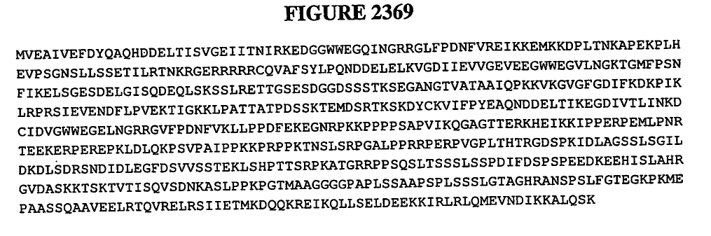

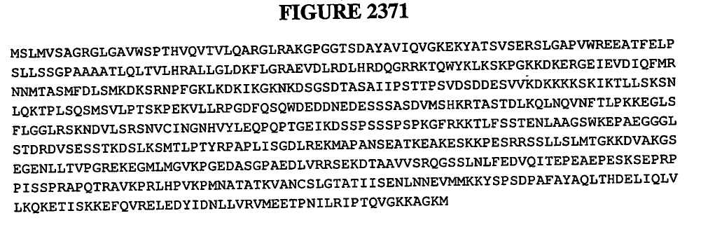

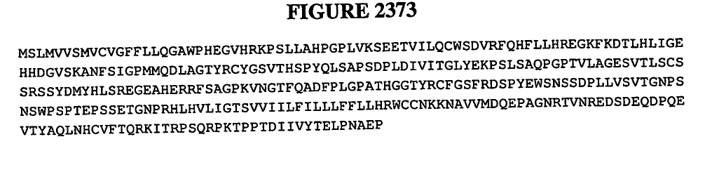

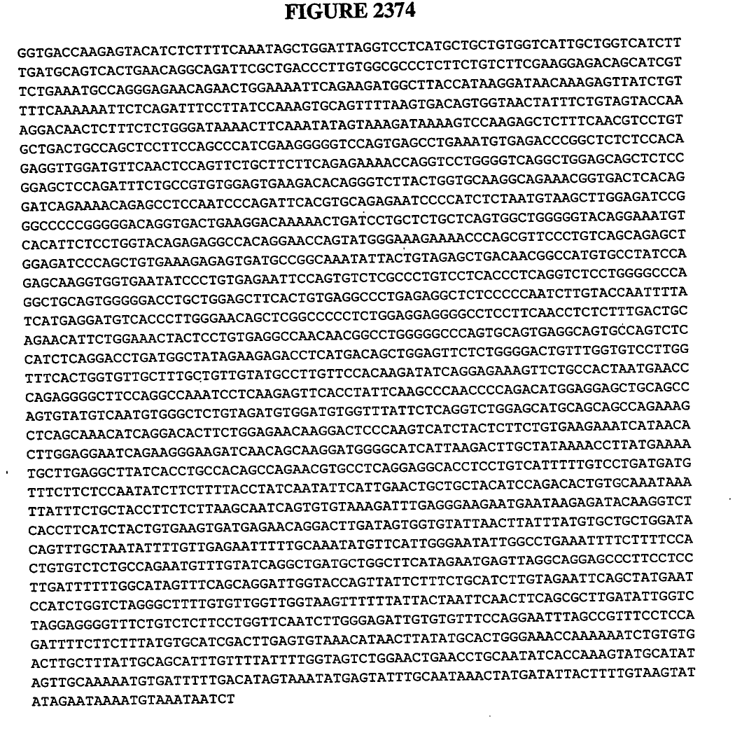

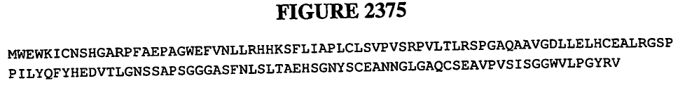

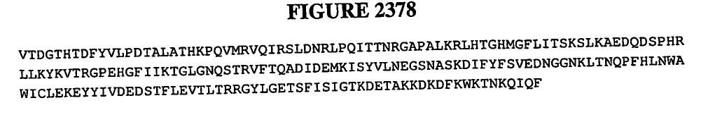

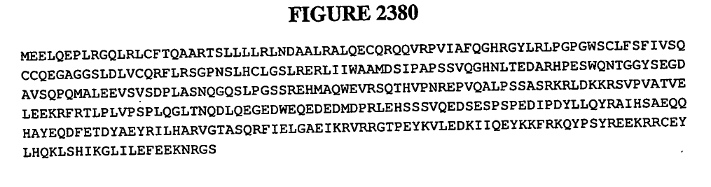

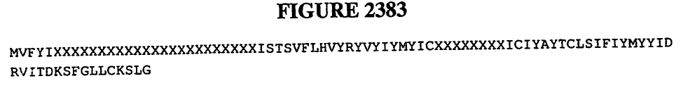

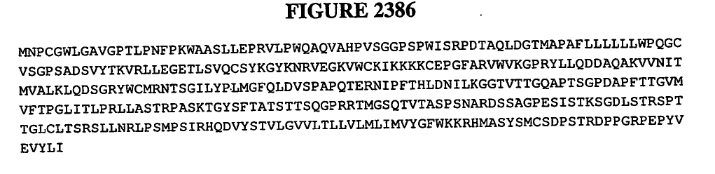

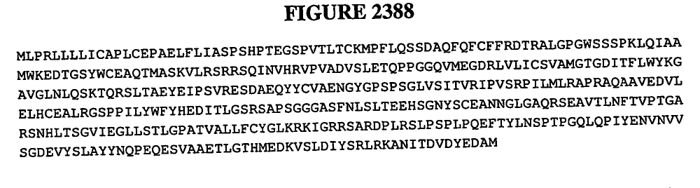

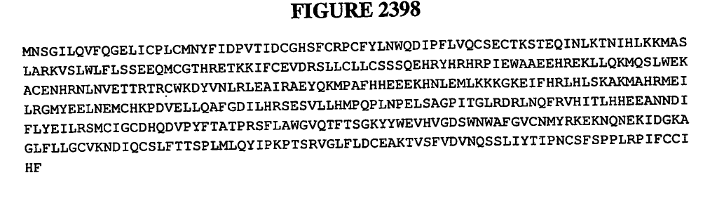

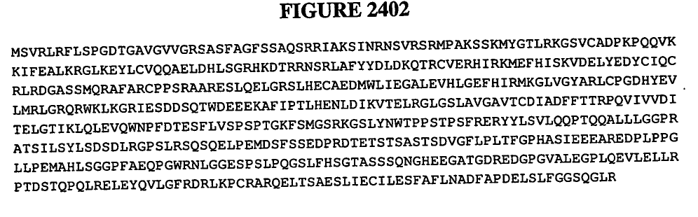

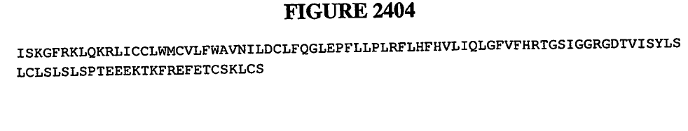

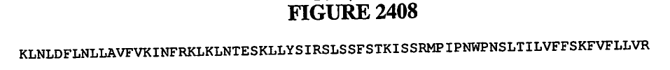

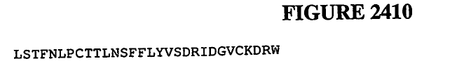

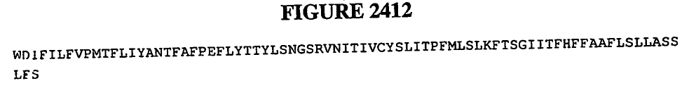

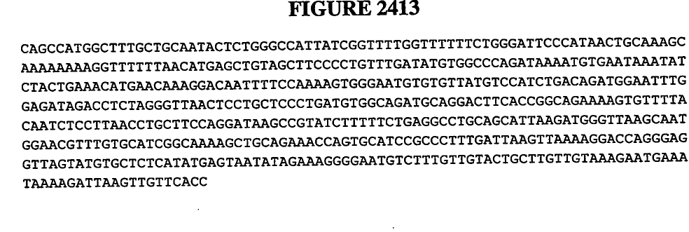

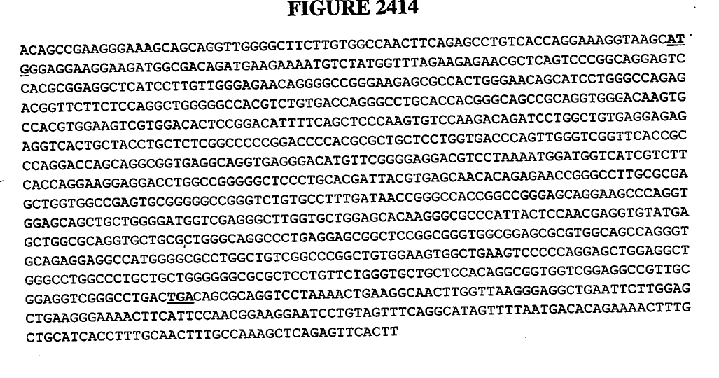

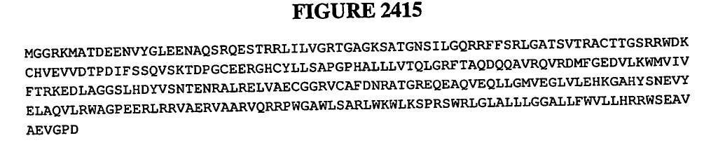

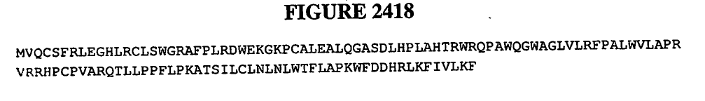

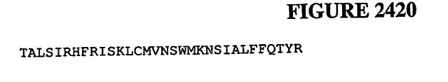

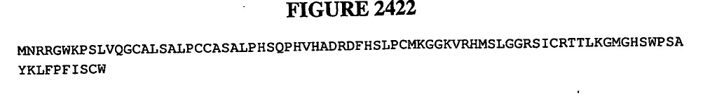

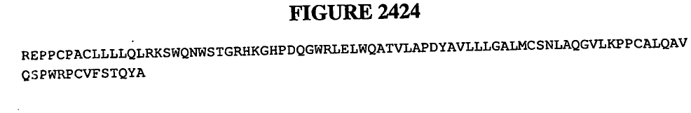

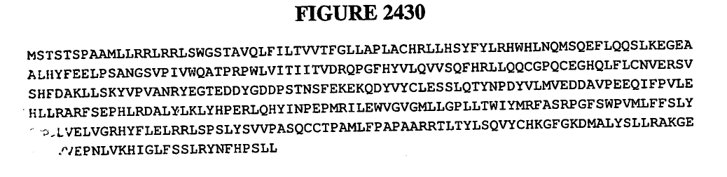

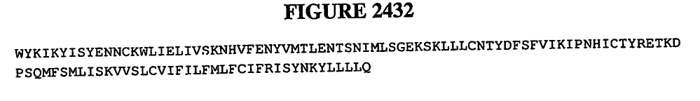

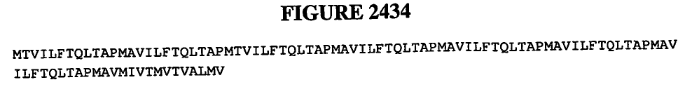

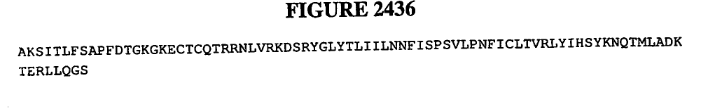

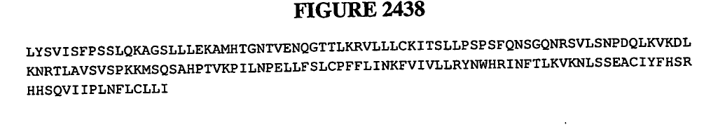

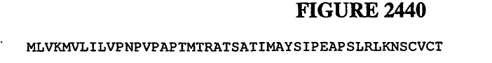

1. Isolated nucleic acid having at least 80% nucleic acid sequence identity to a nucleotide sequence encoding the polypeptide shown in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646); FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442).

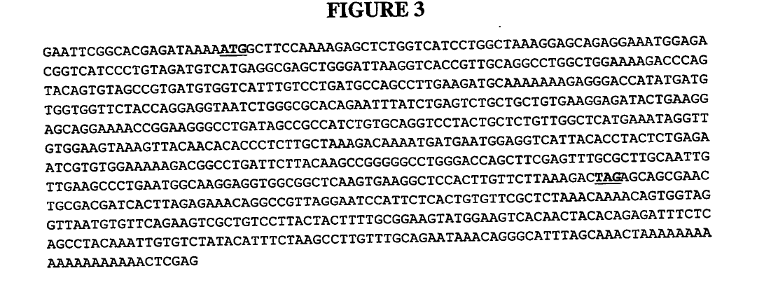

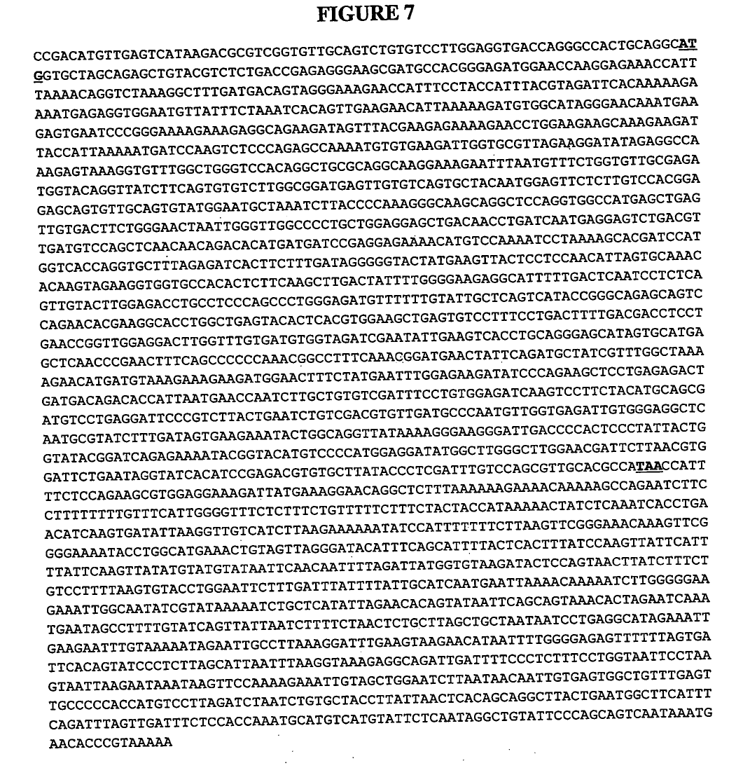

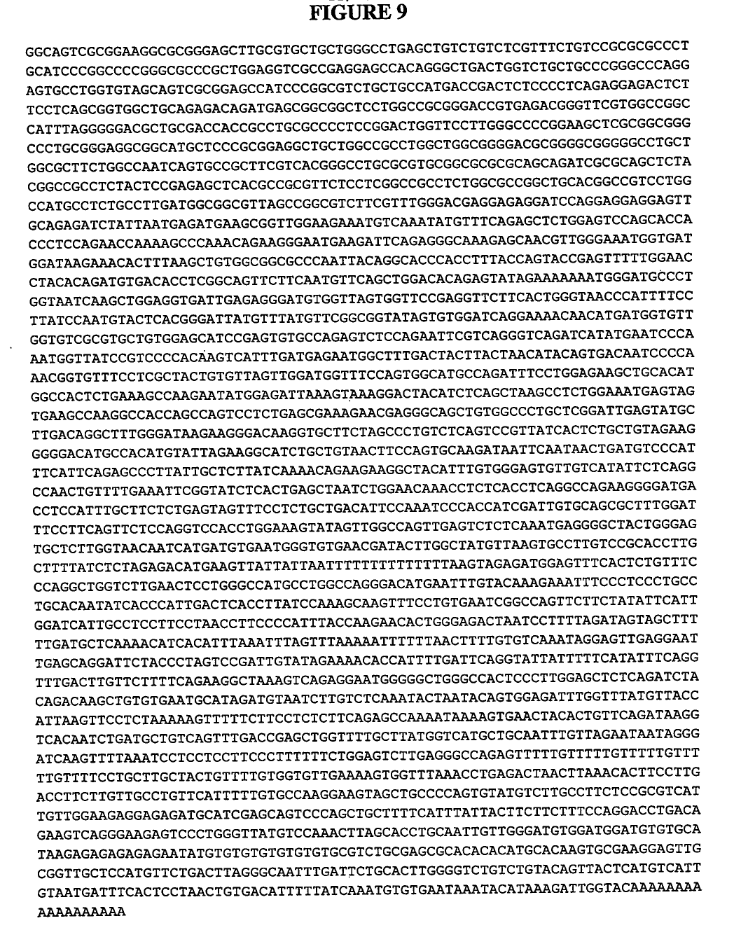

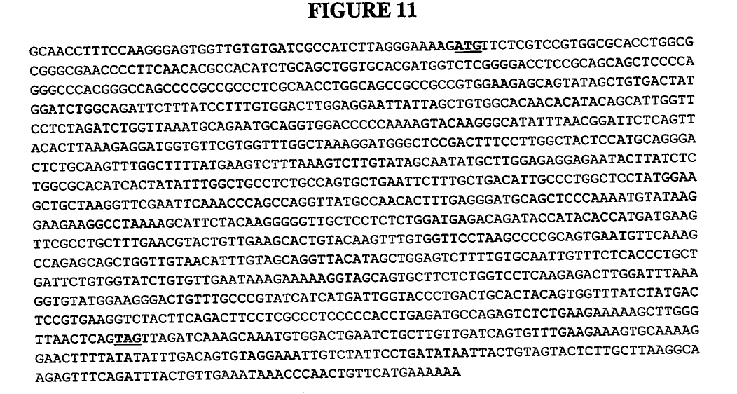

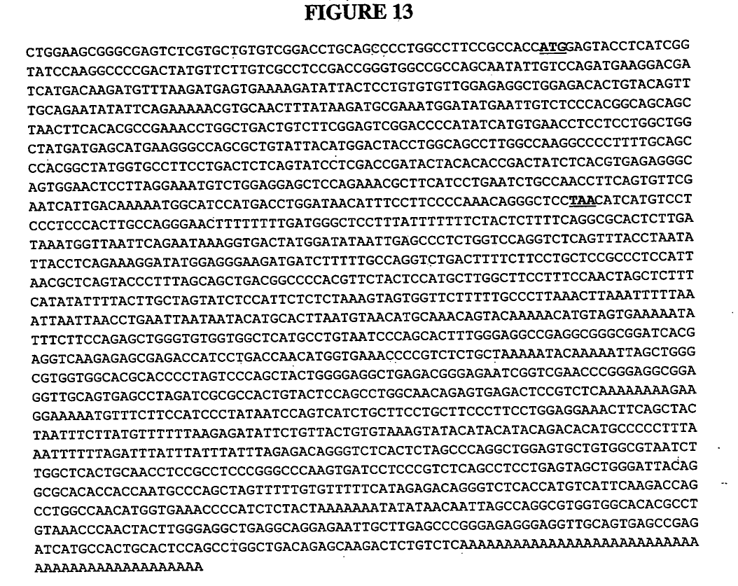

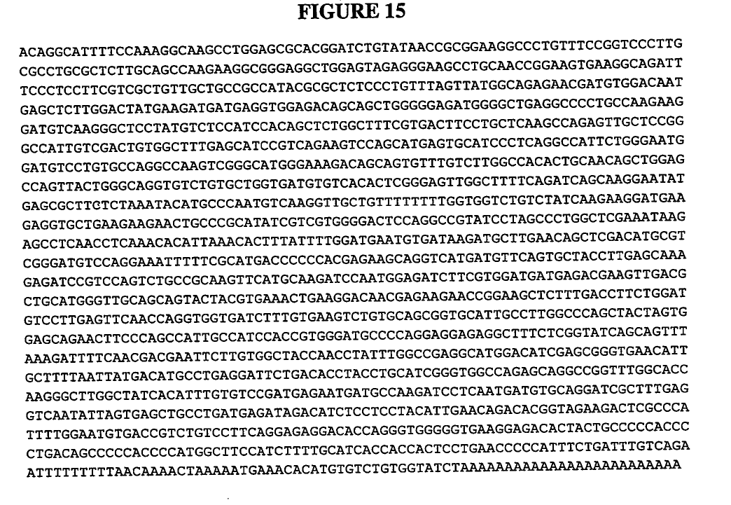

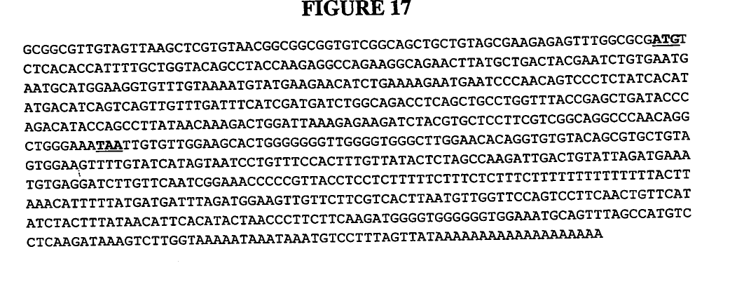

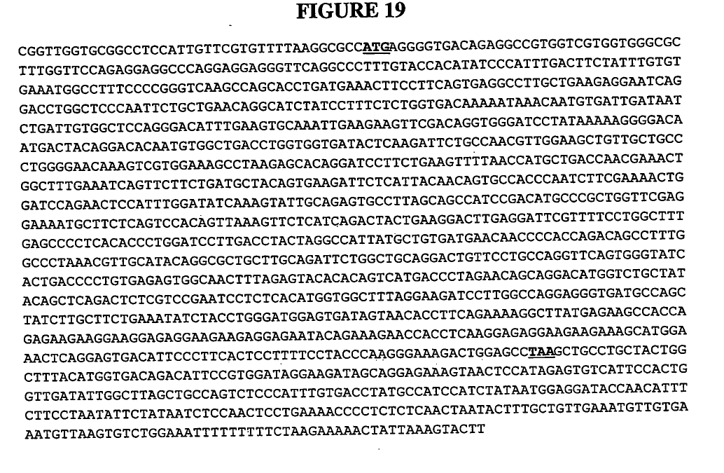

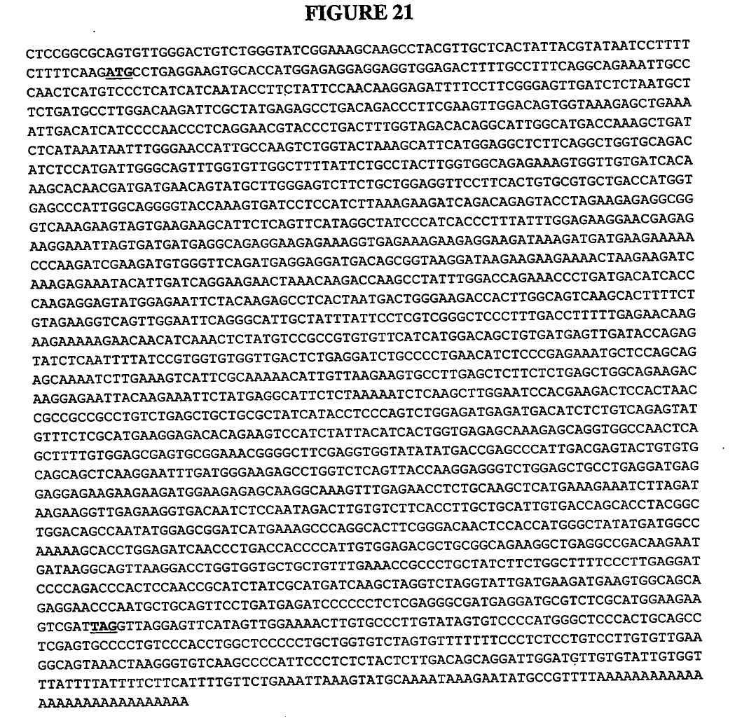

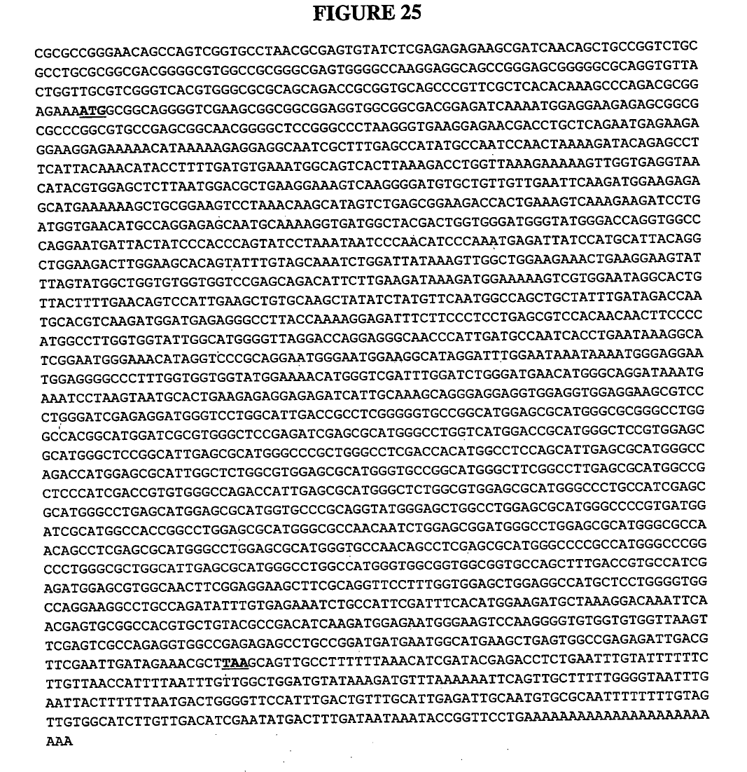

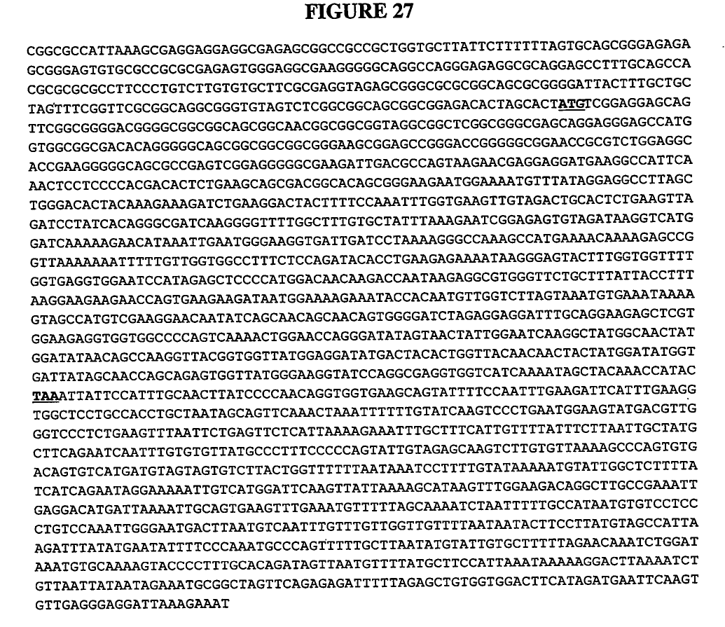

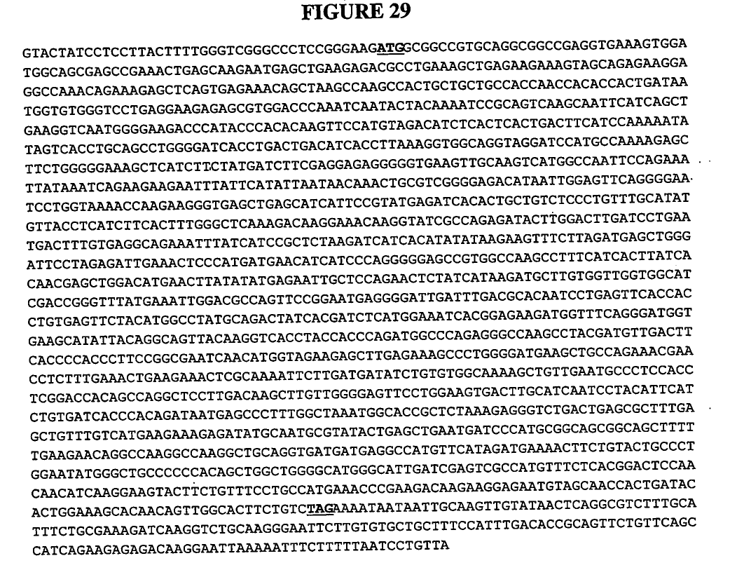

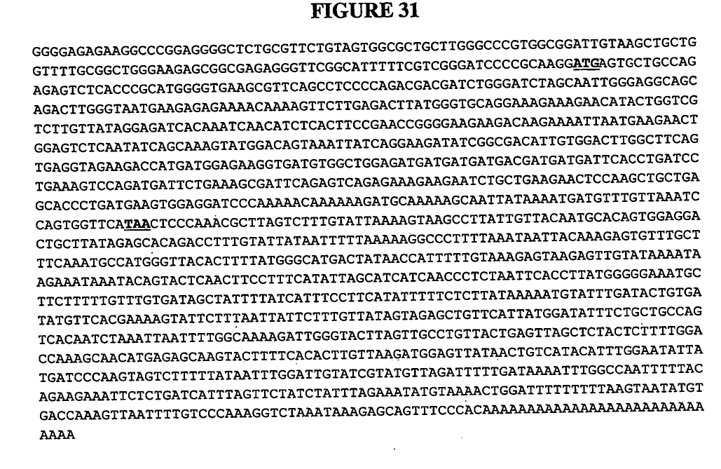

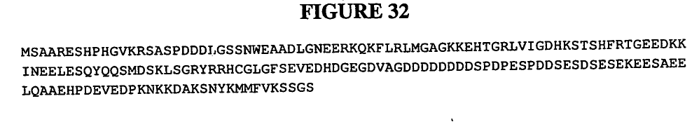

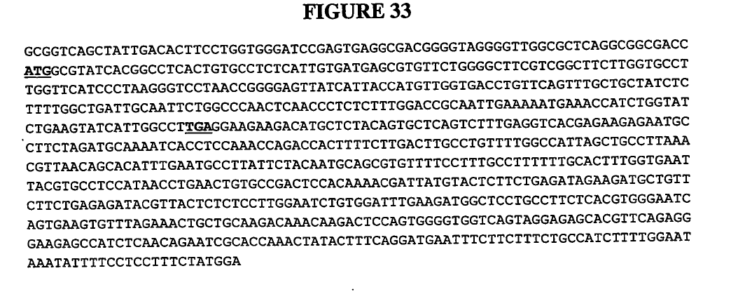

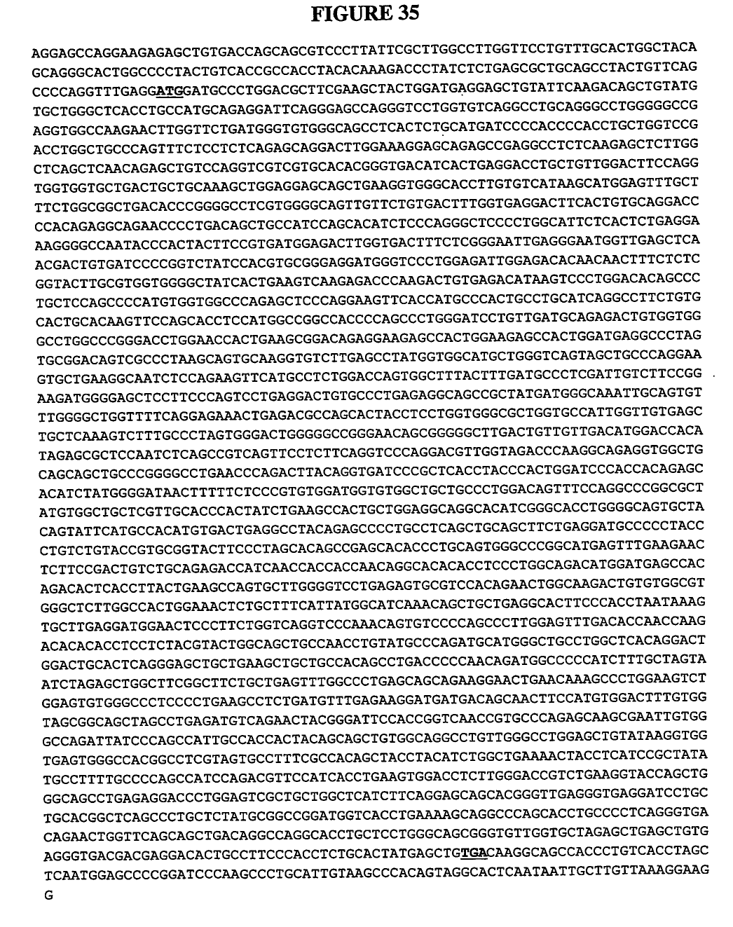

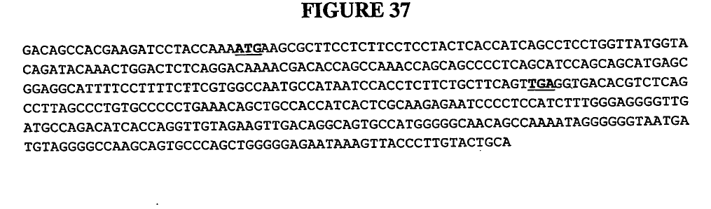

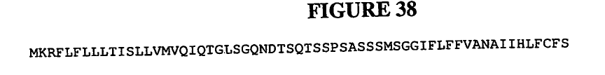

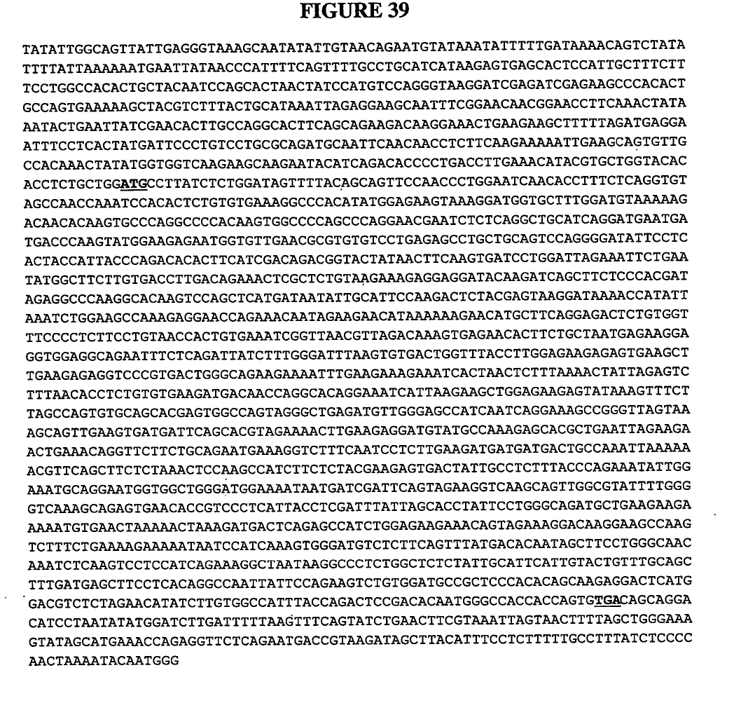

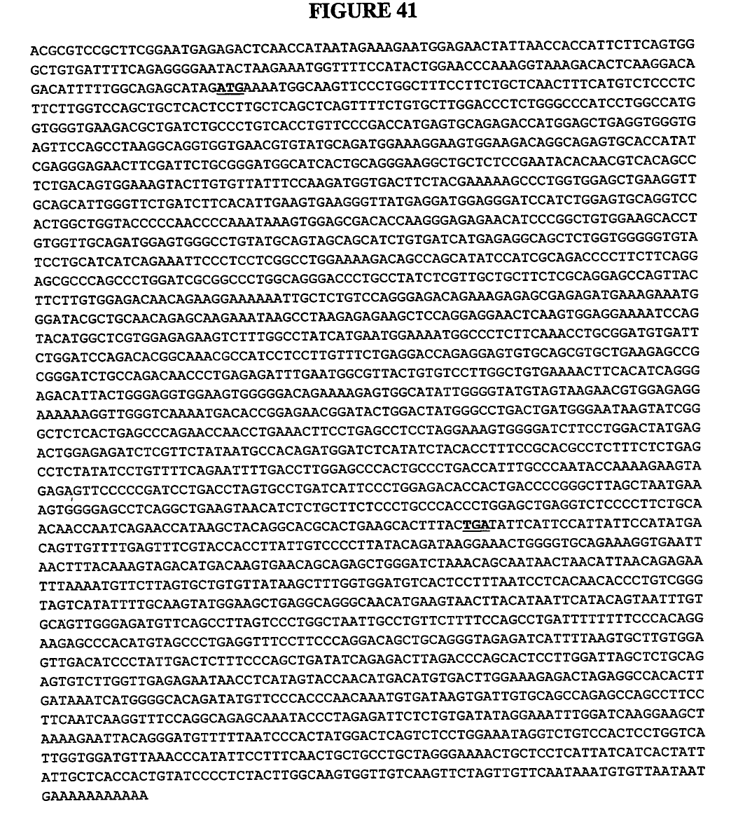

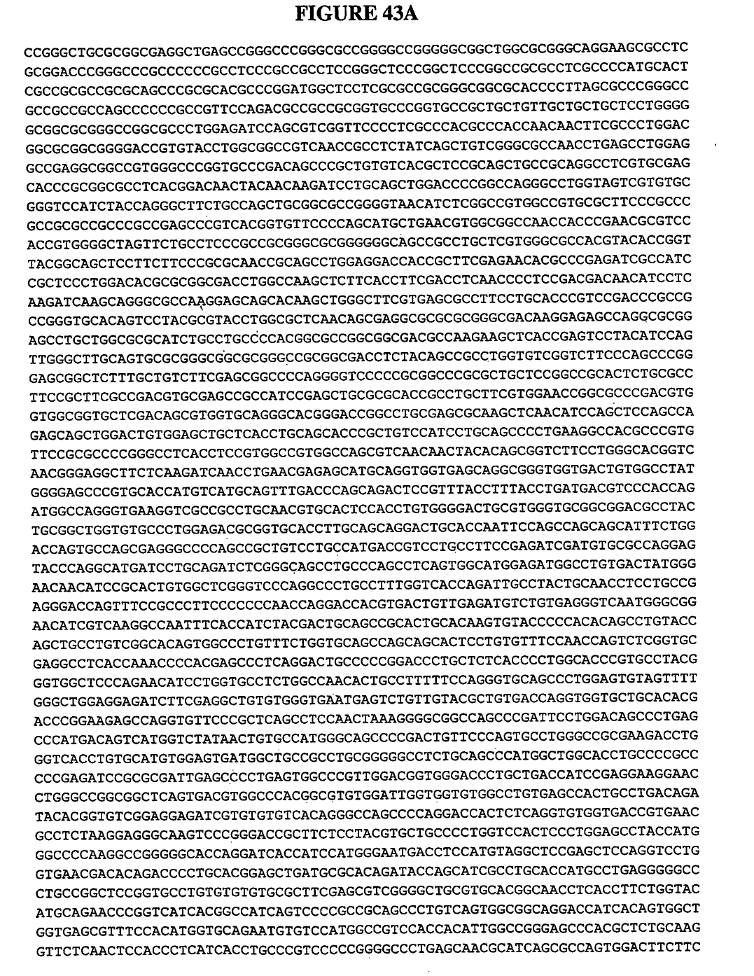

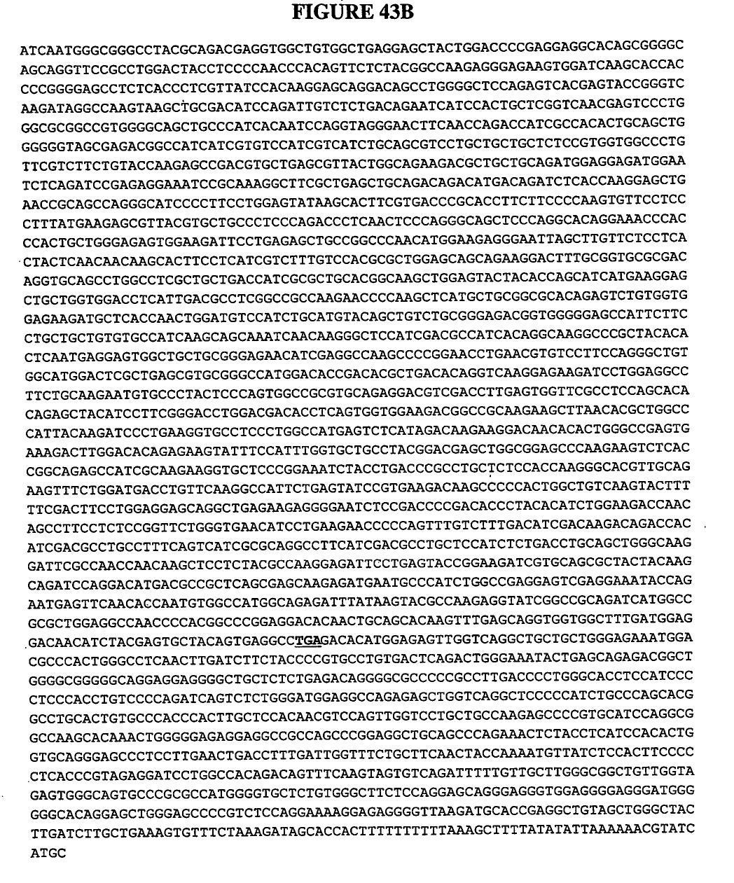

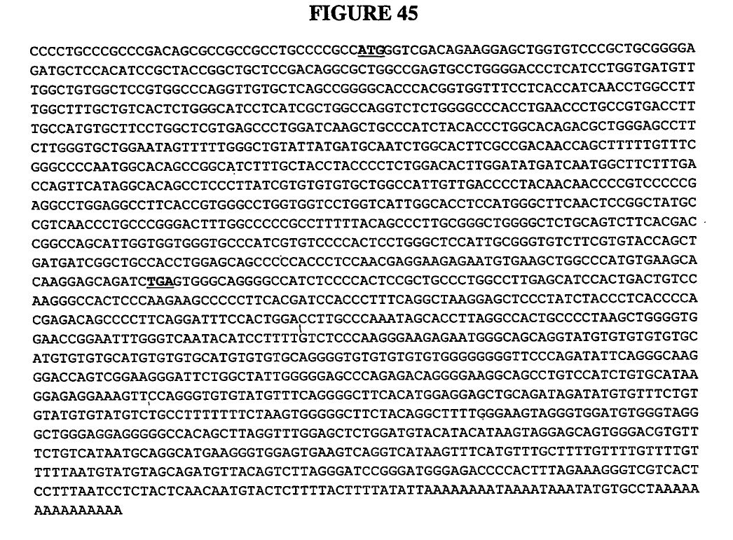

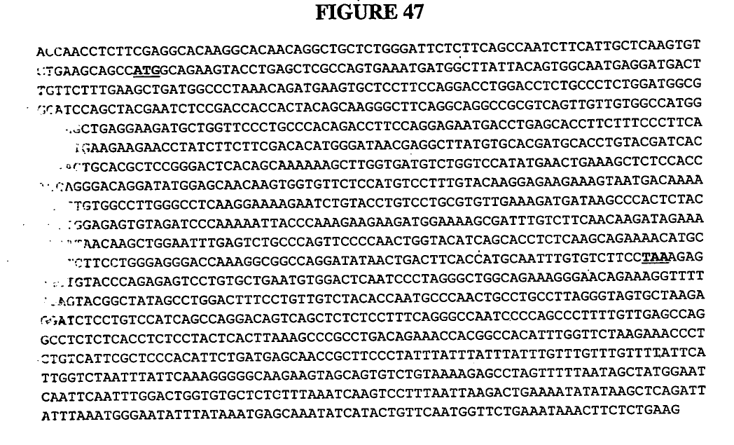

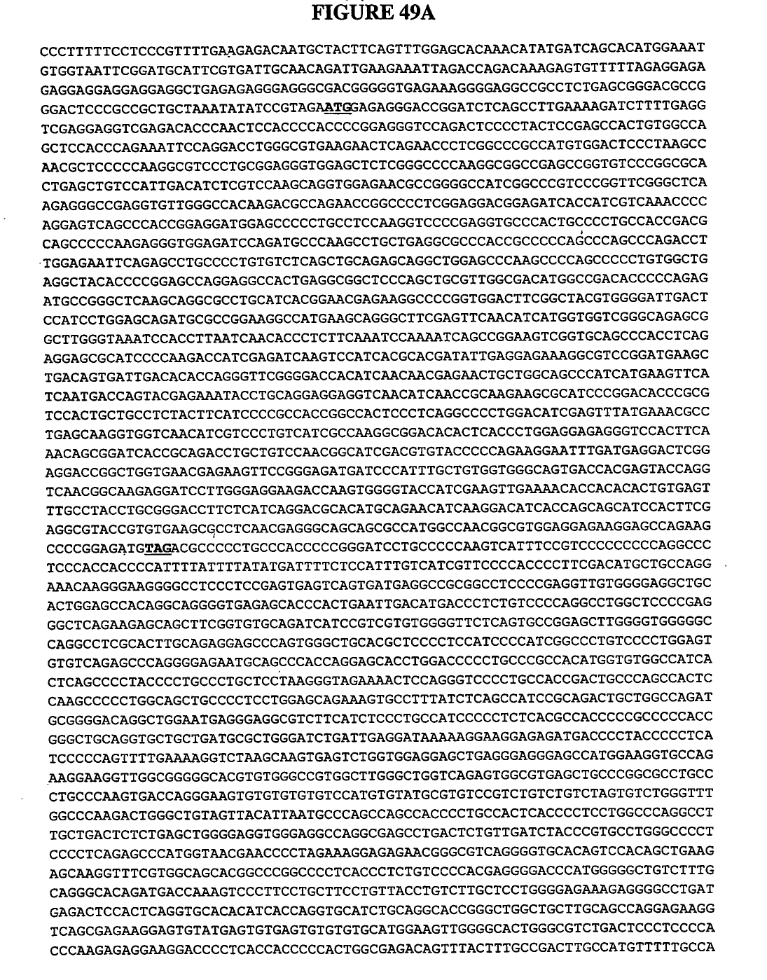

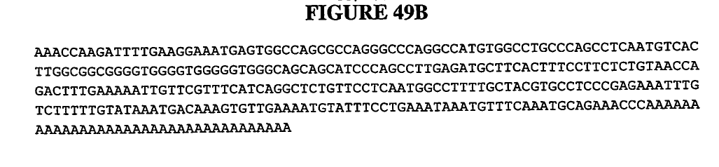

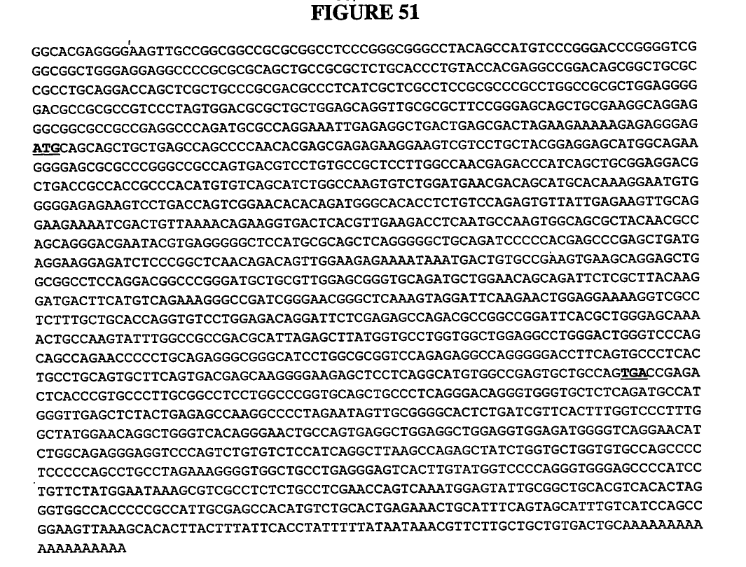

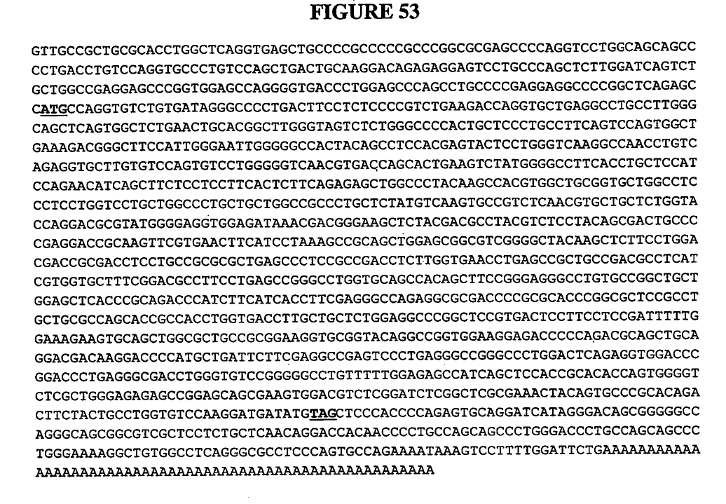

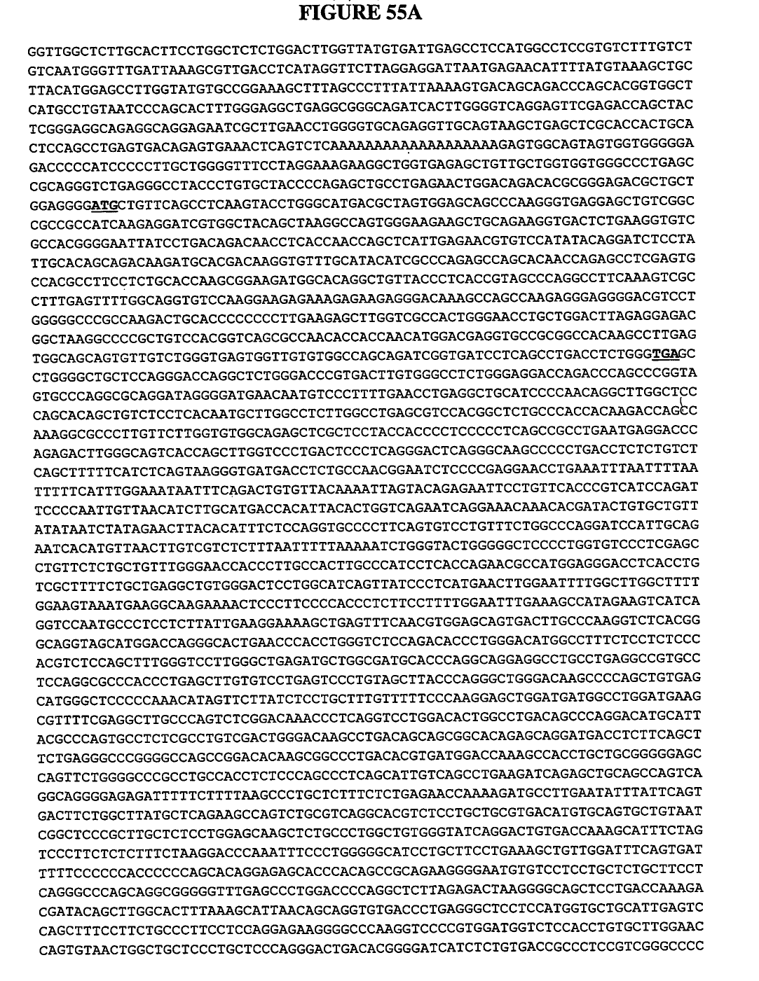

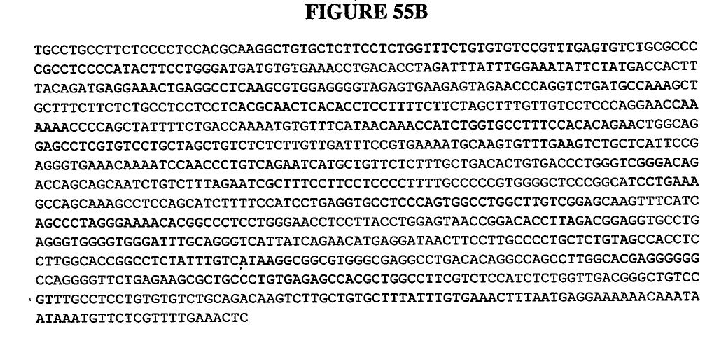

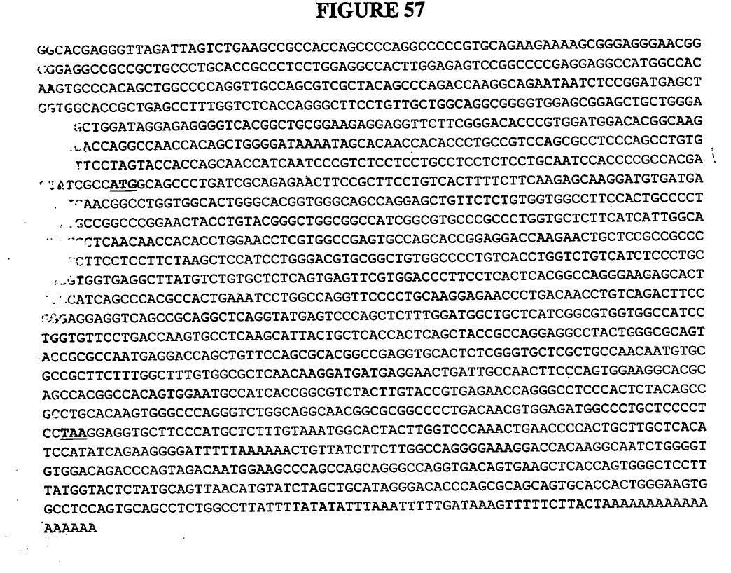

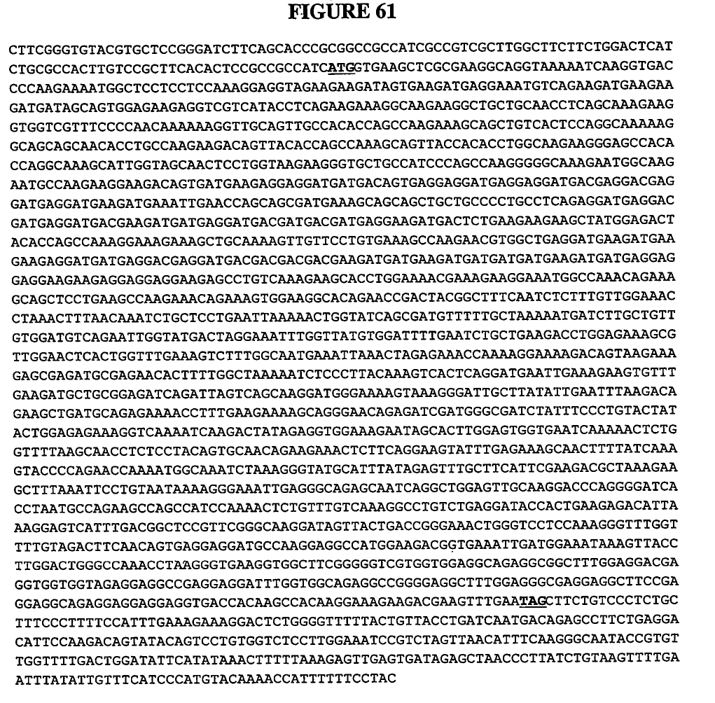

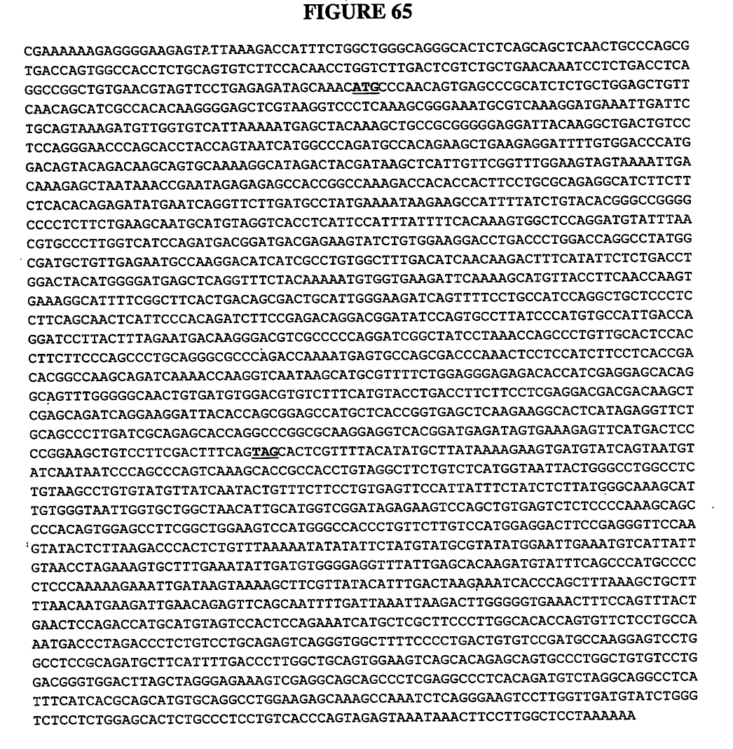

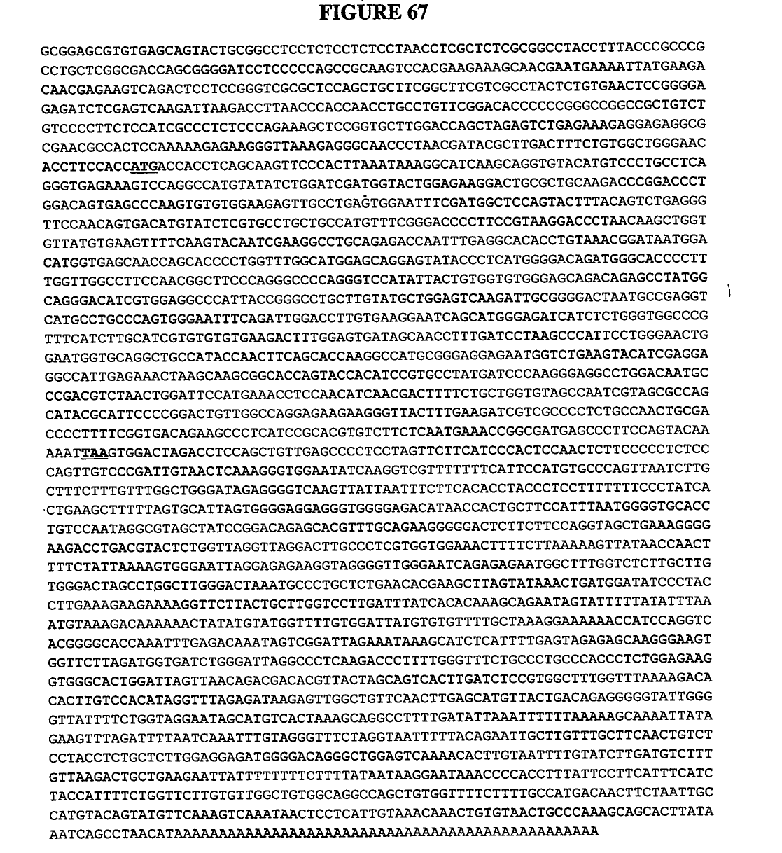

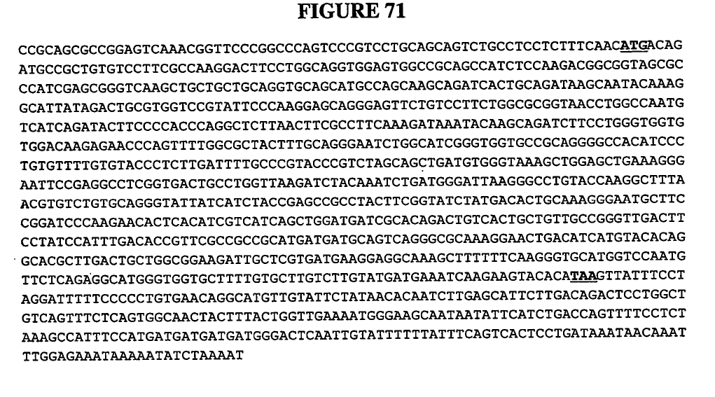

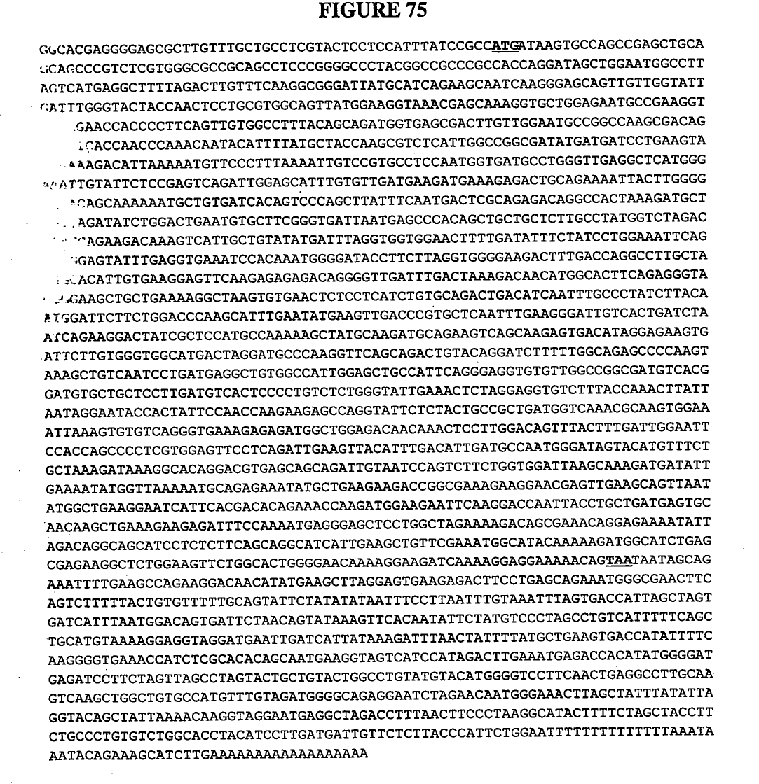

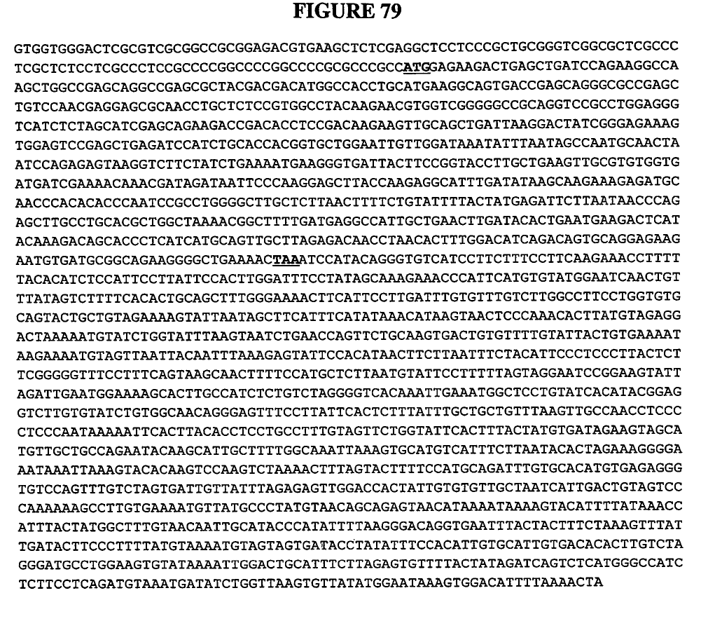

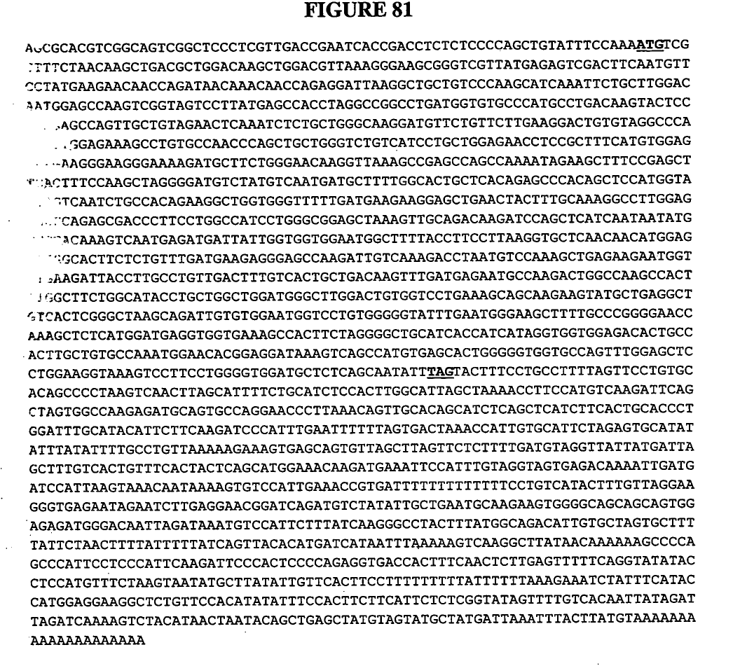

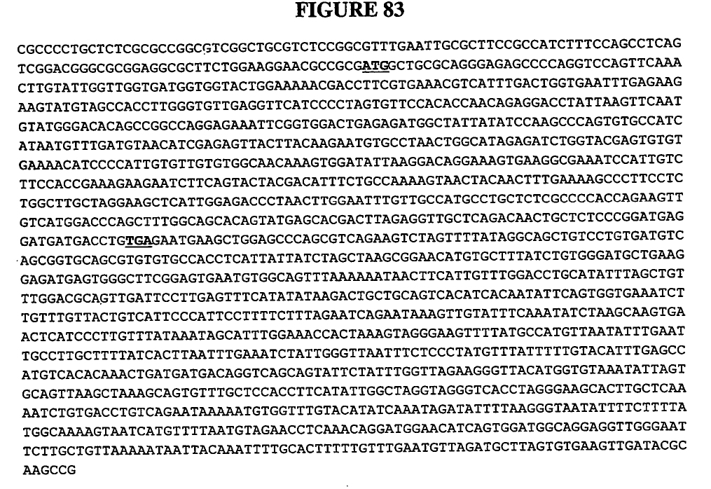

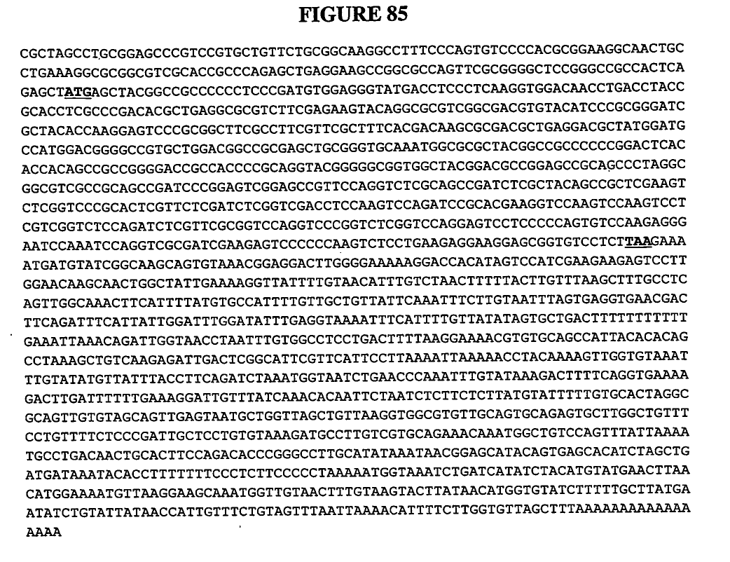

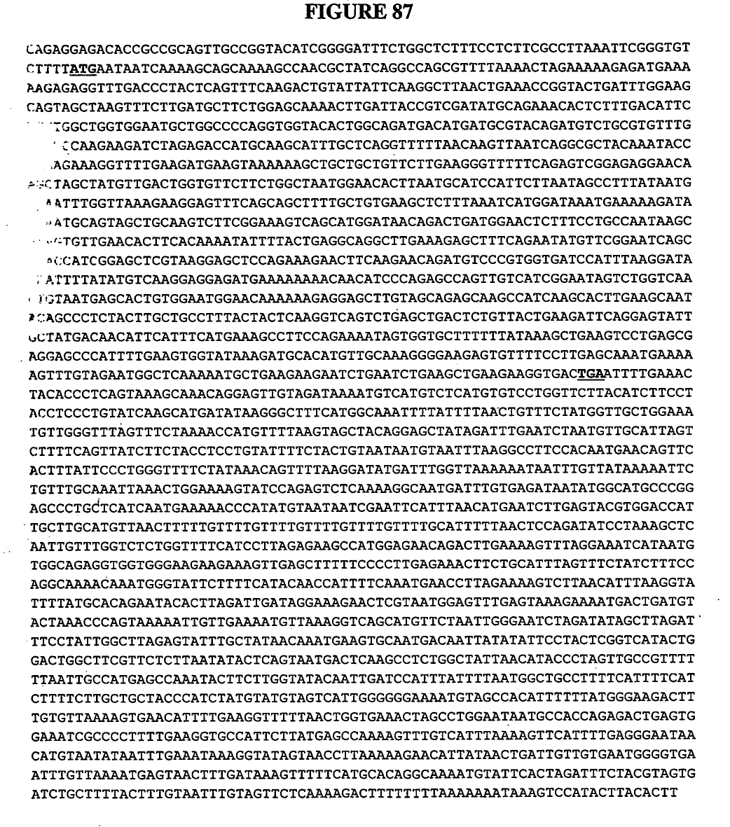

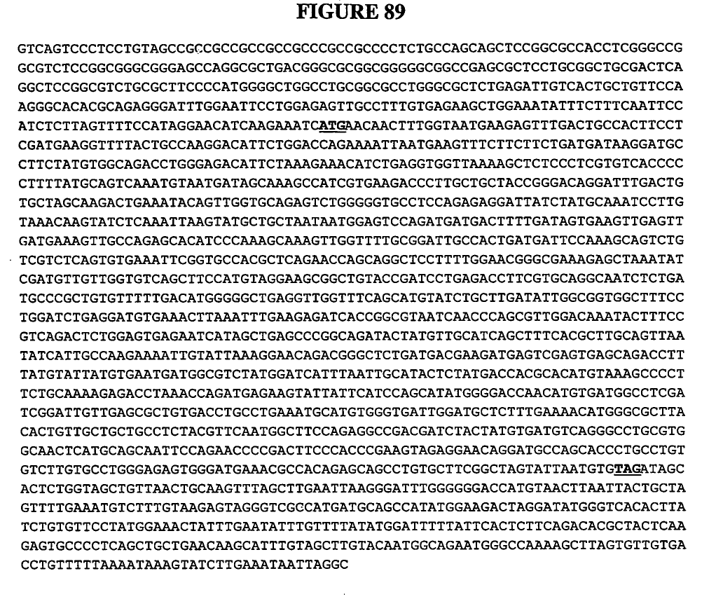

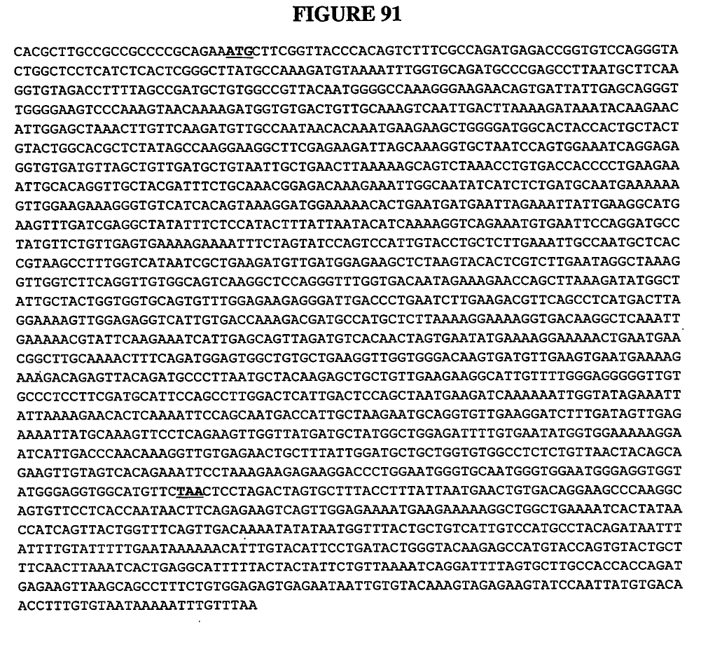

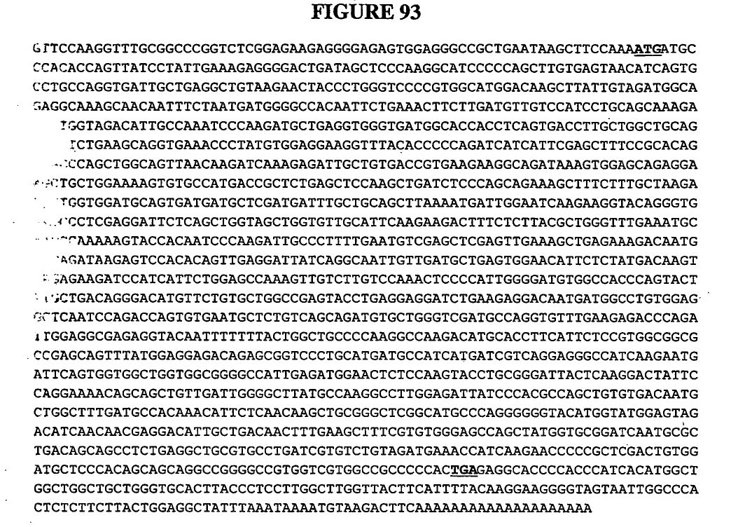

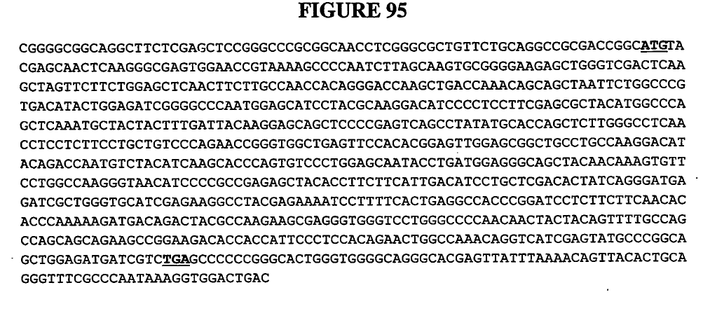

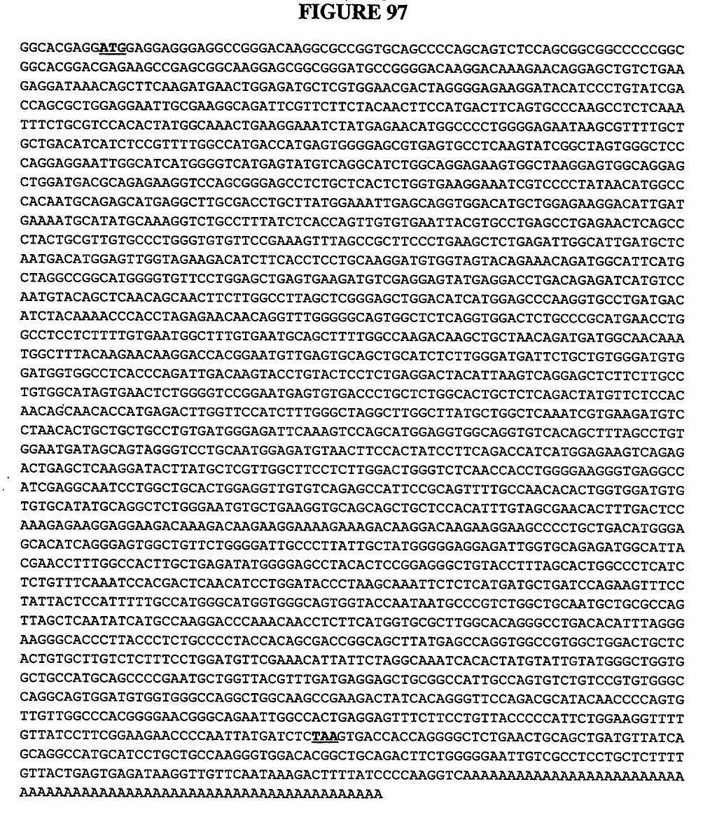

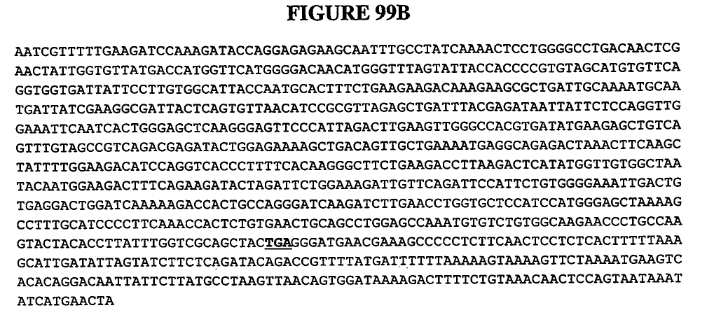

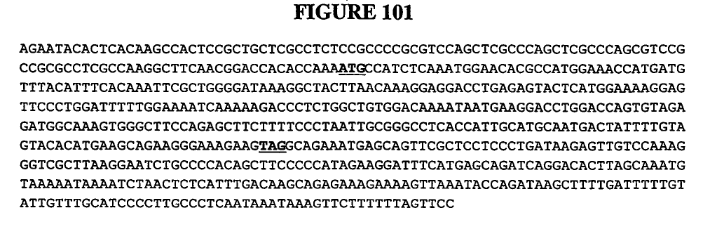

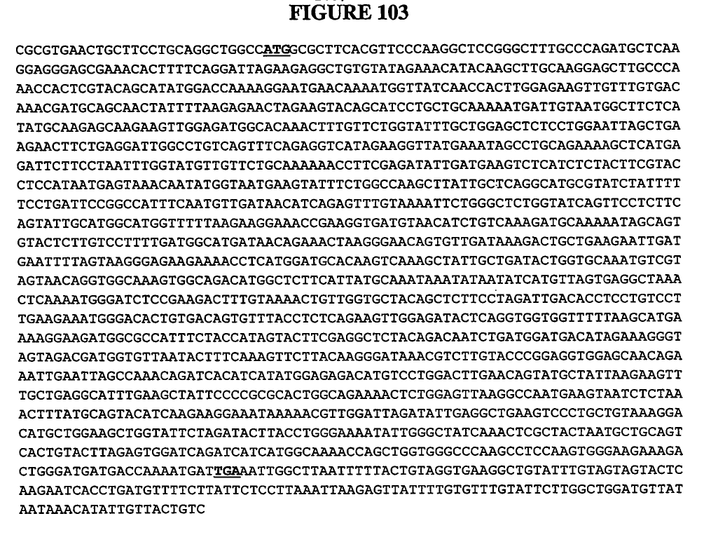

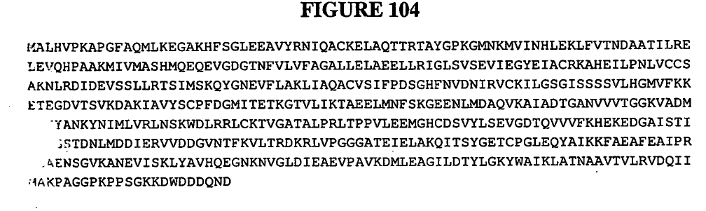

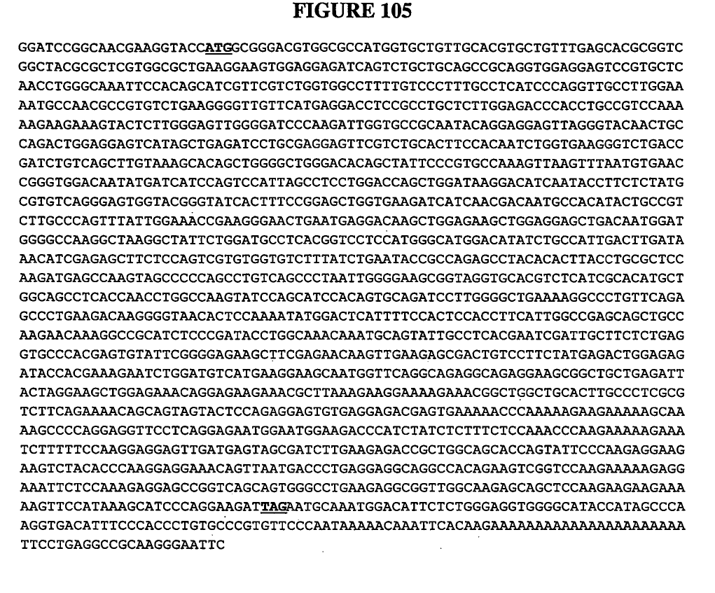

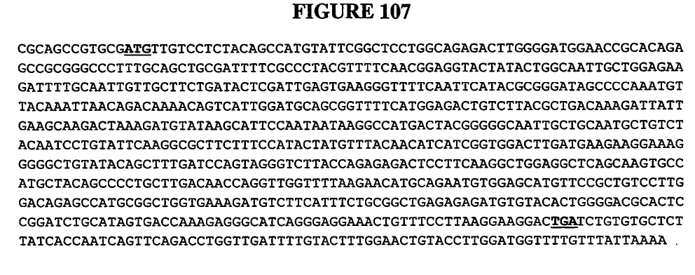

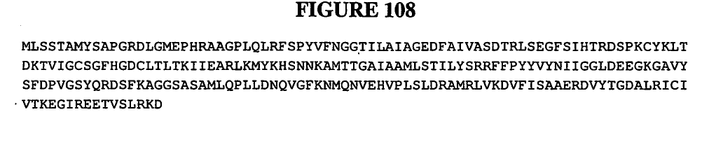

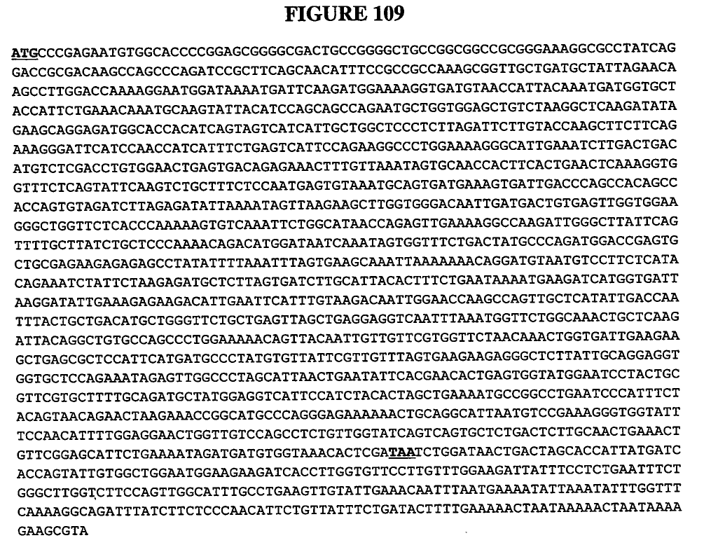

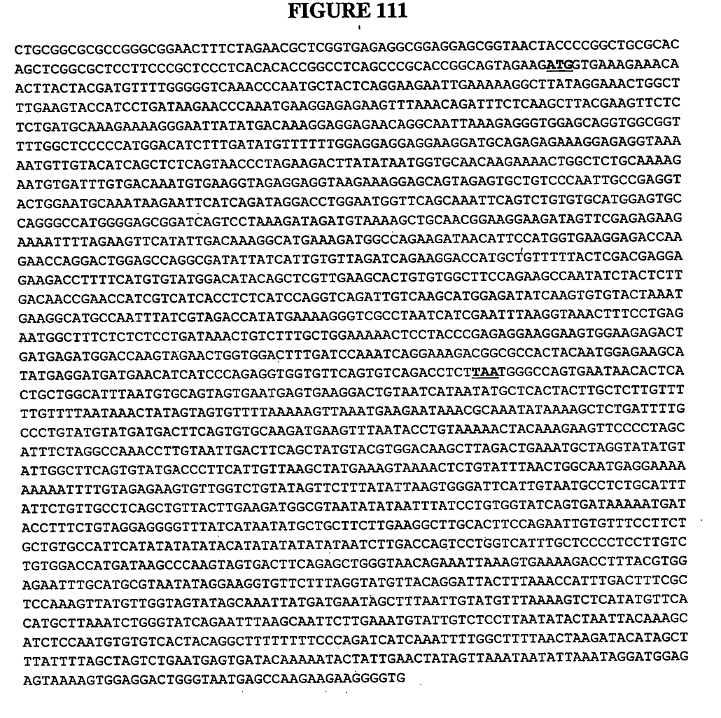

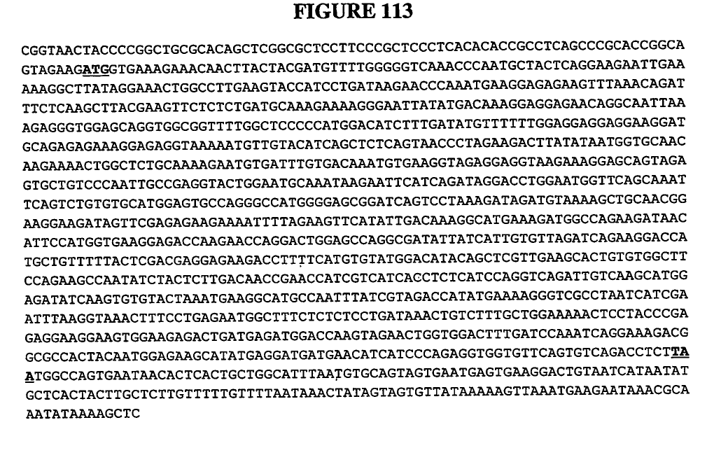

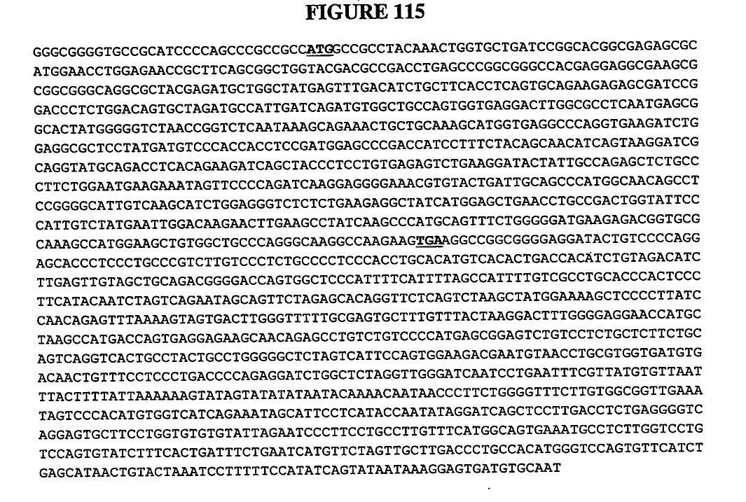

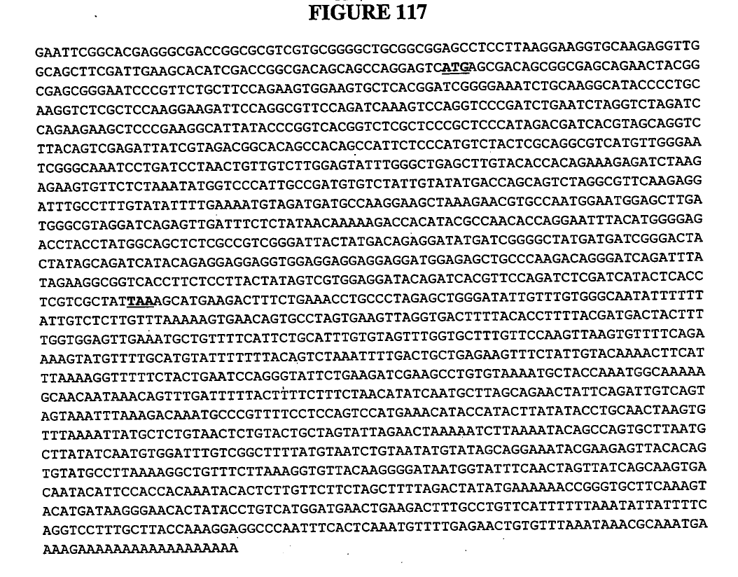

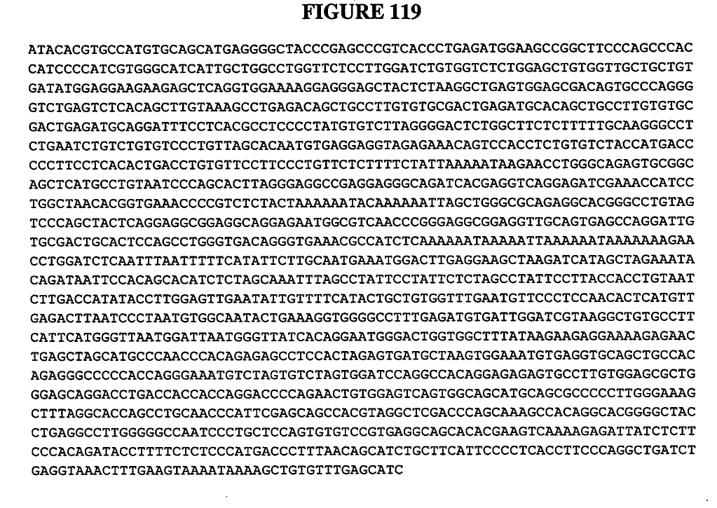

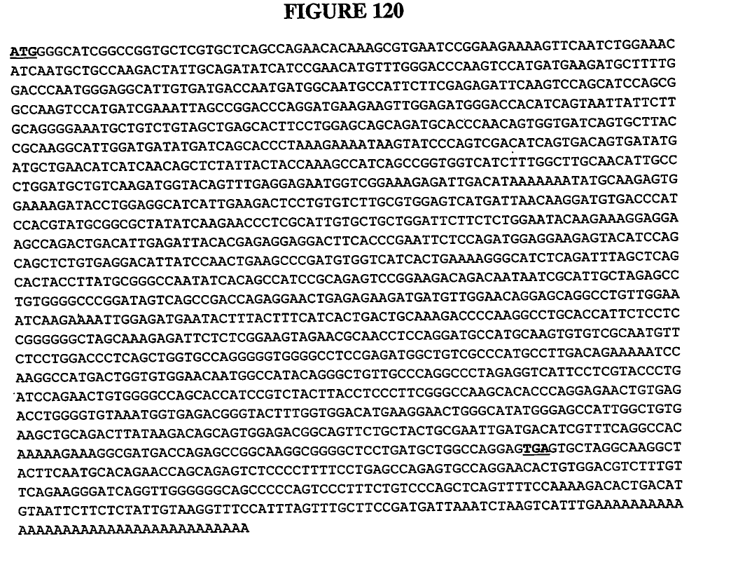

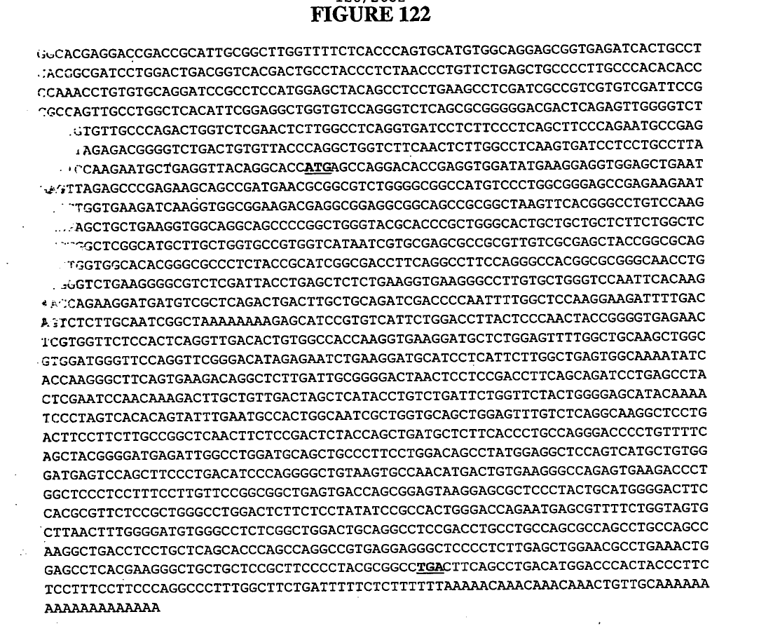

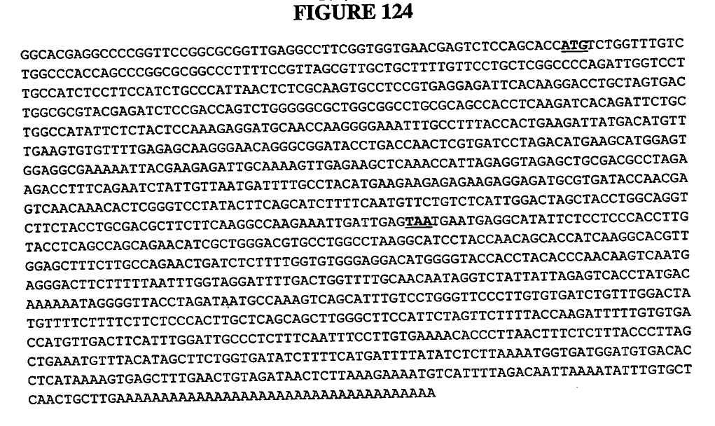

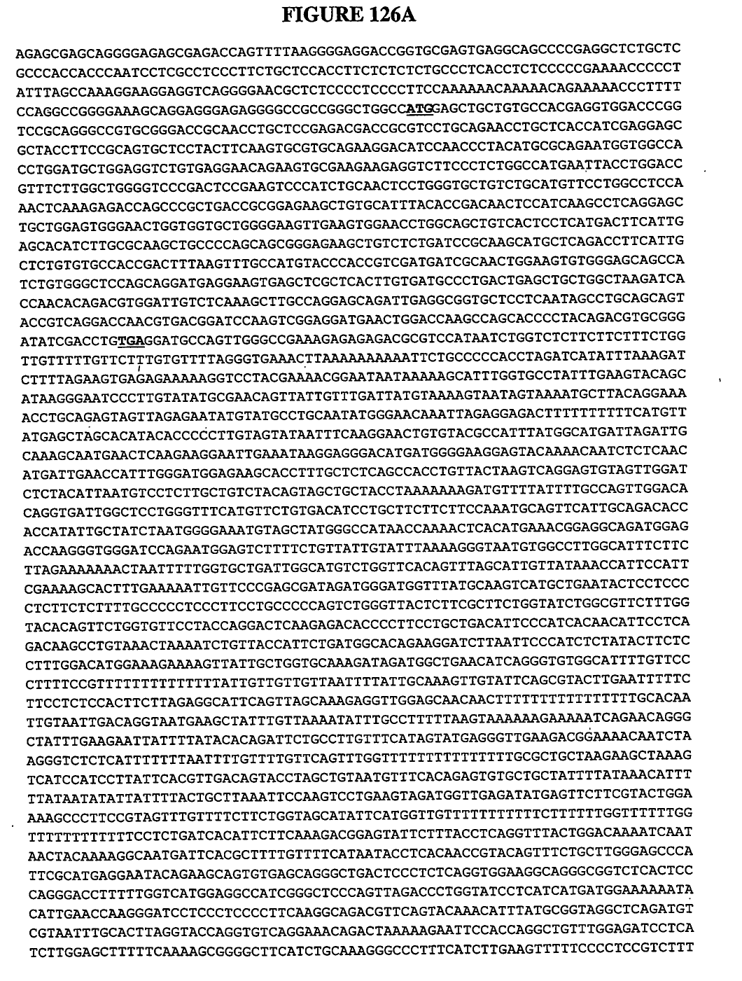

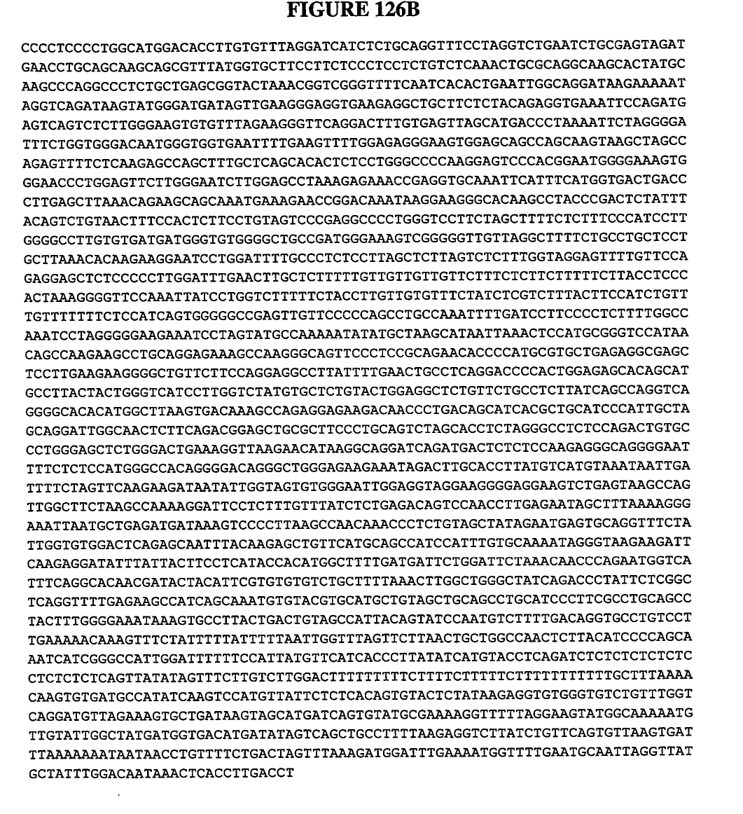

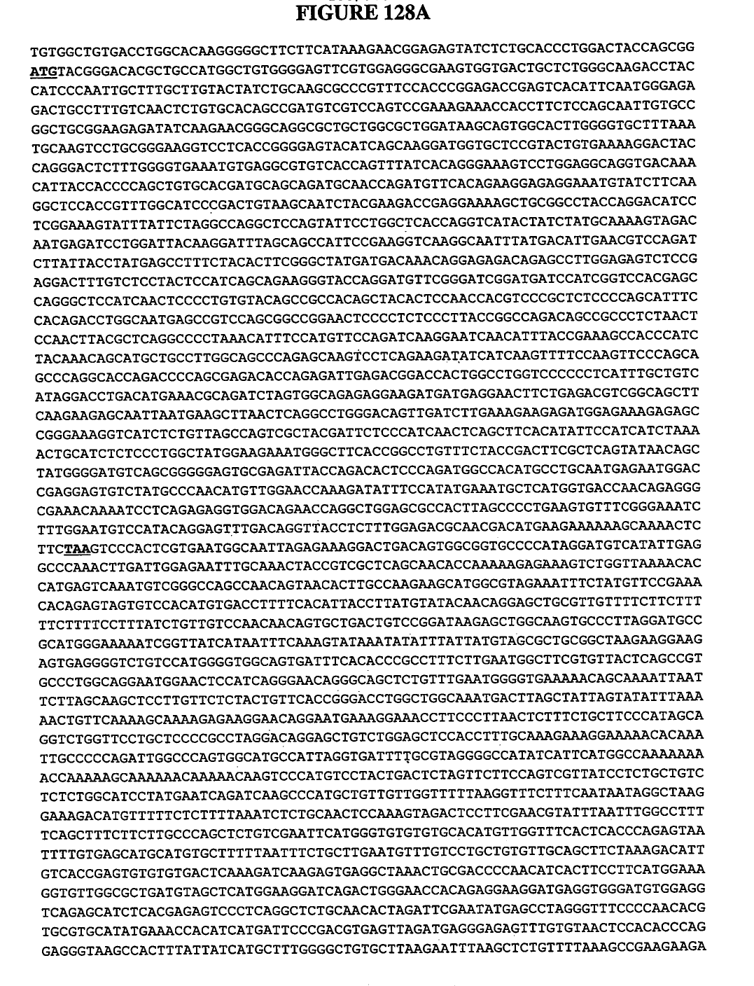

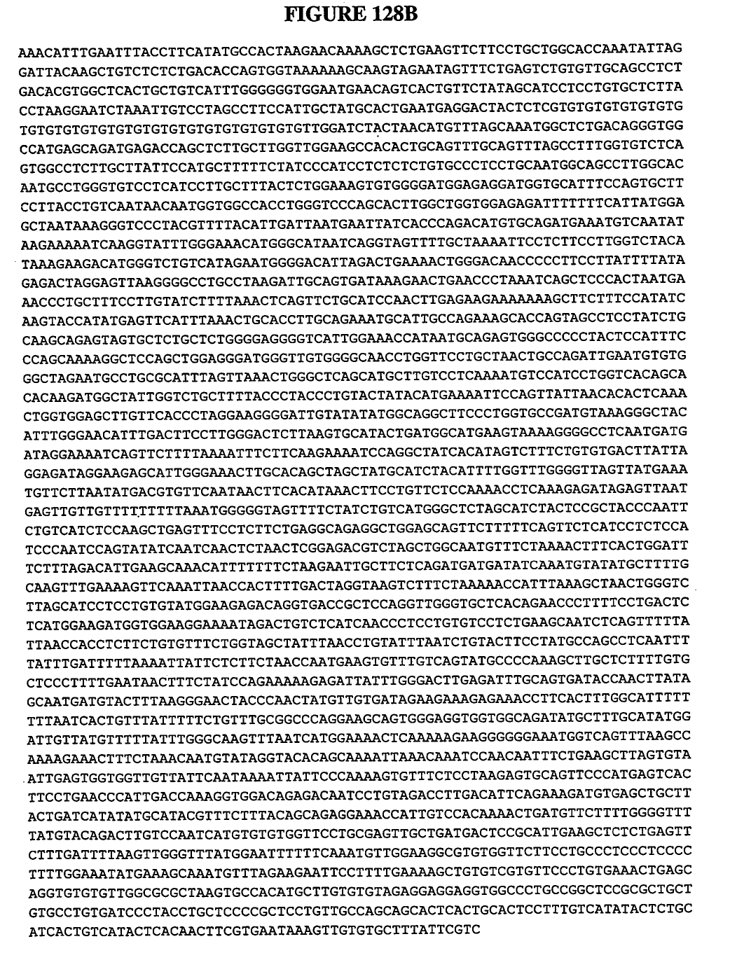

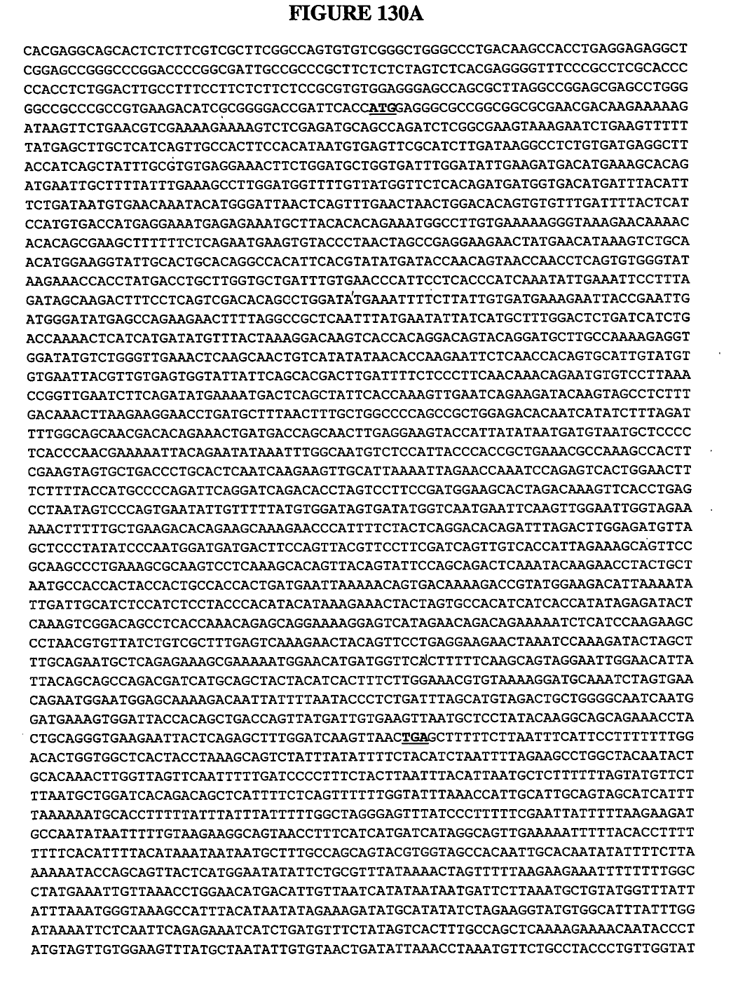

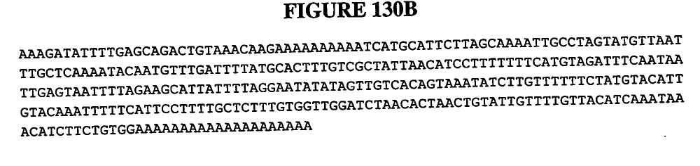

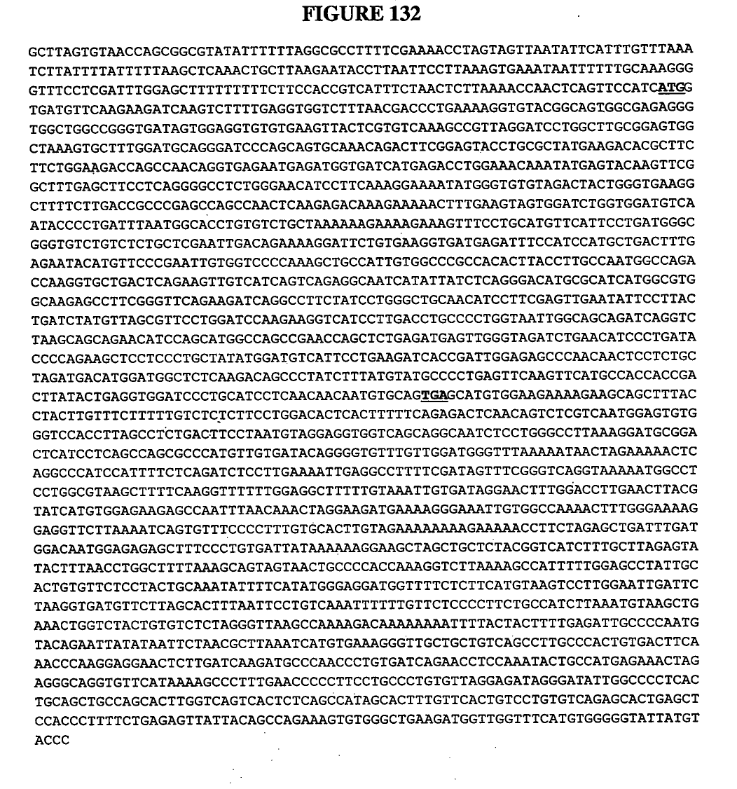

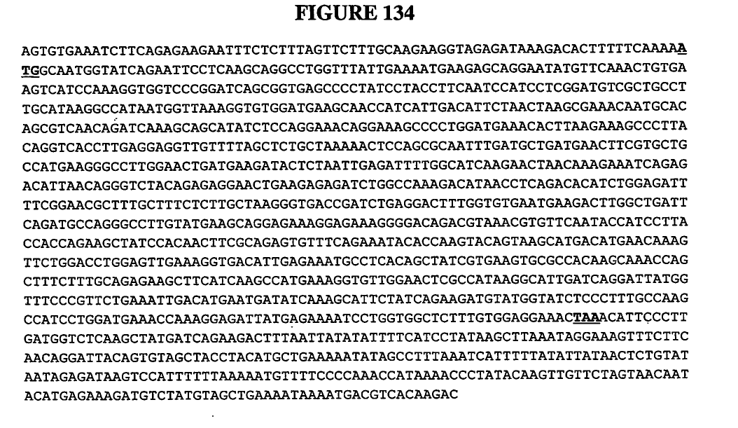

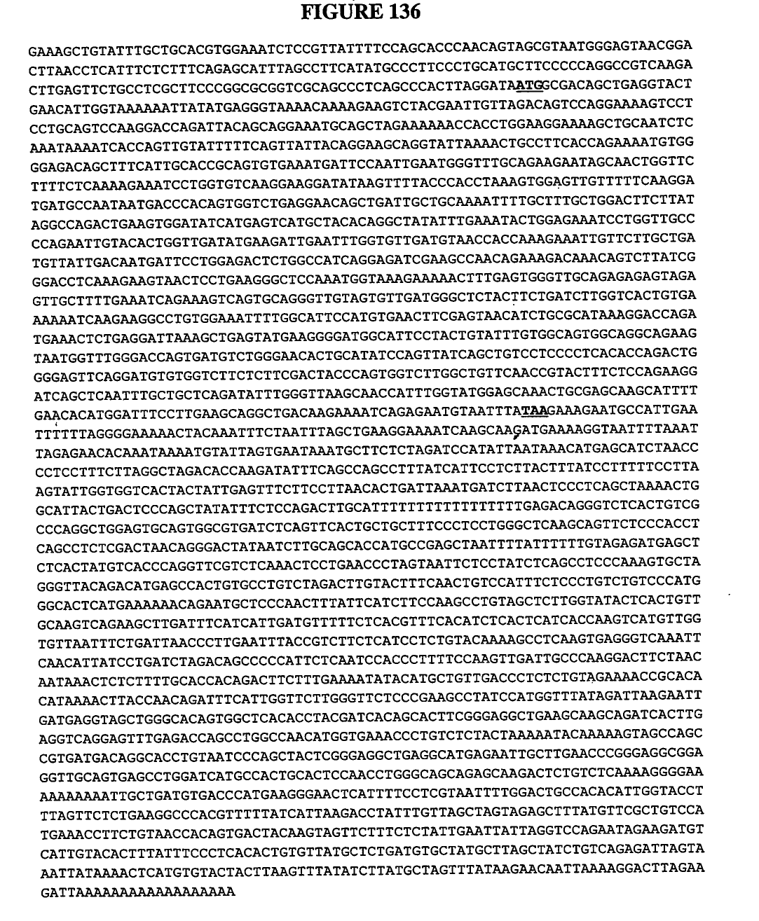

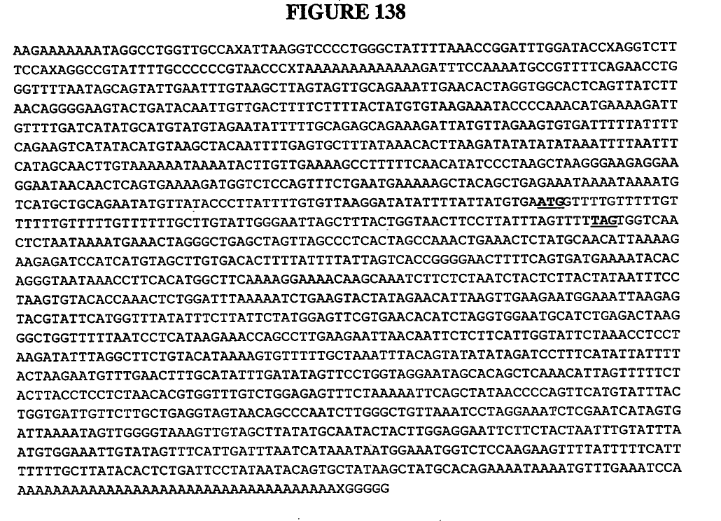

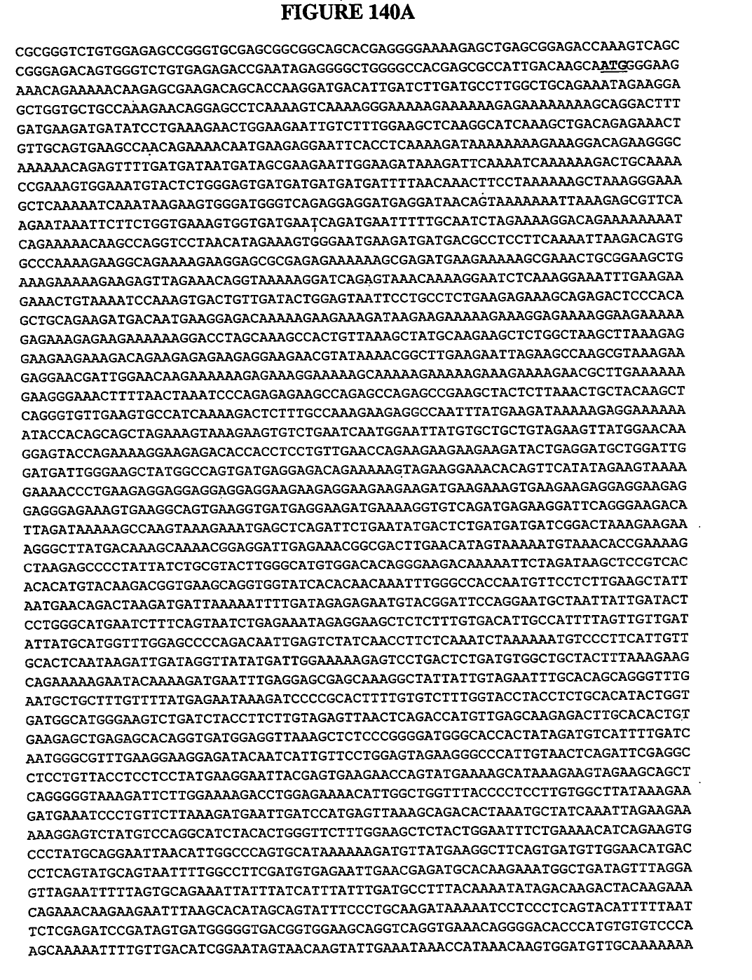

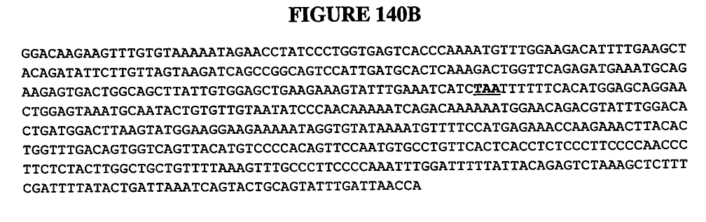

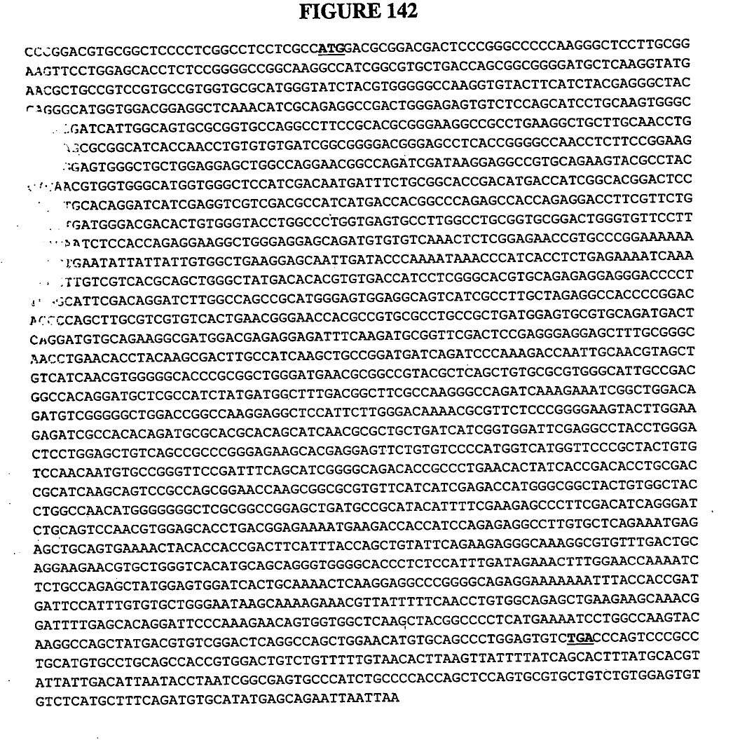

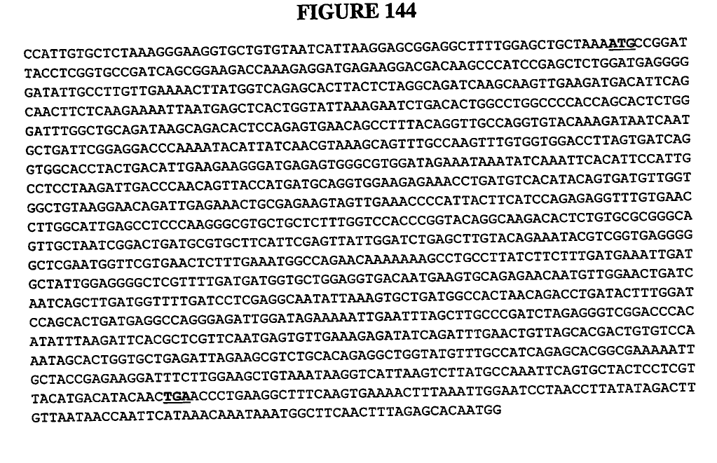

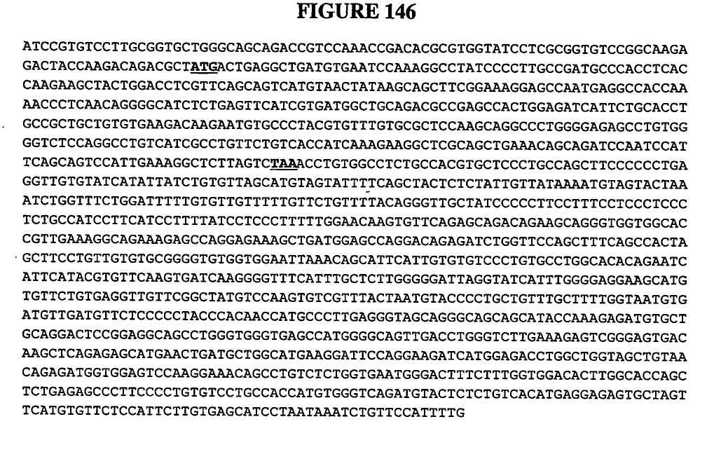

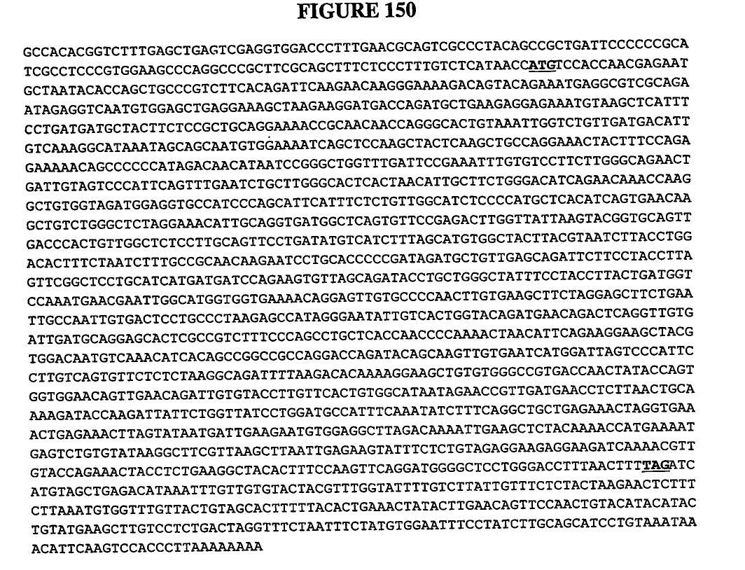

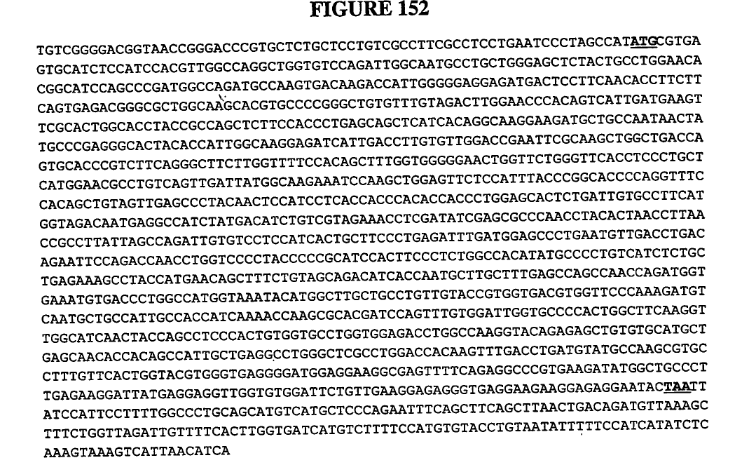

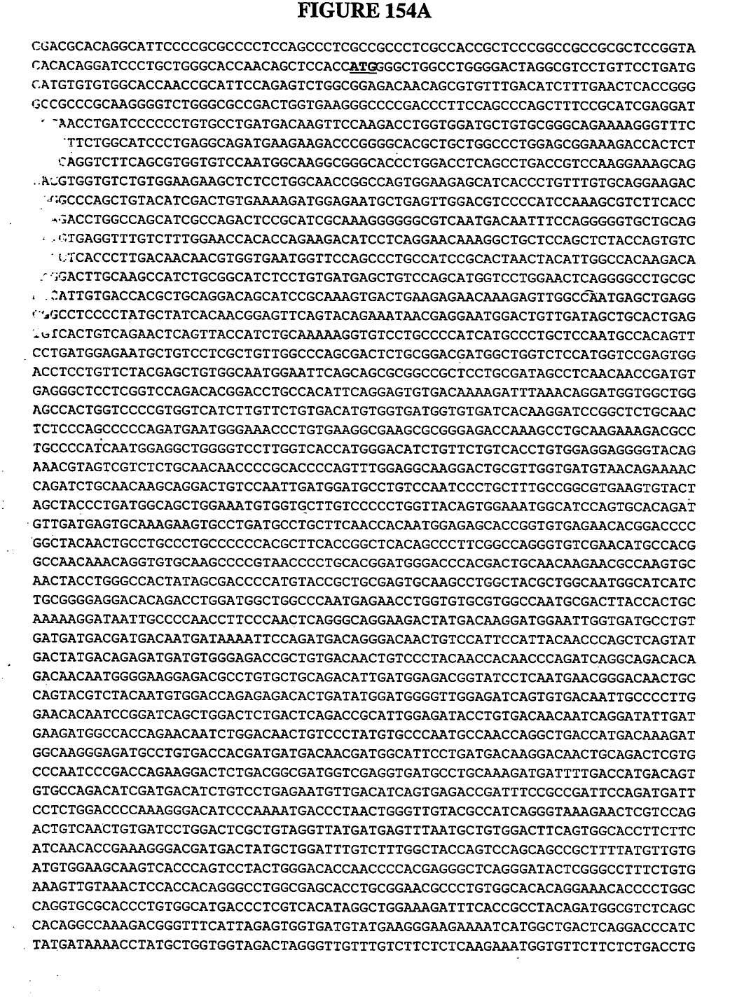

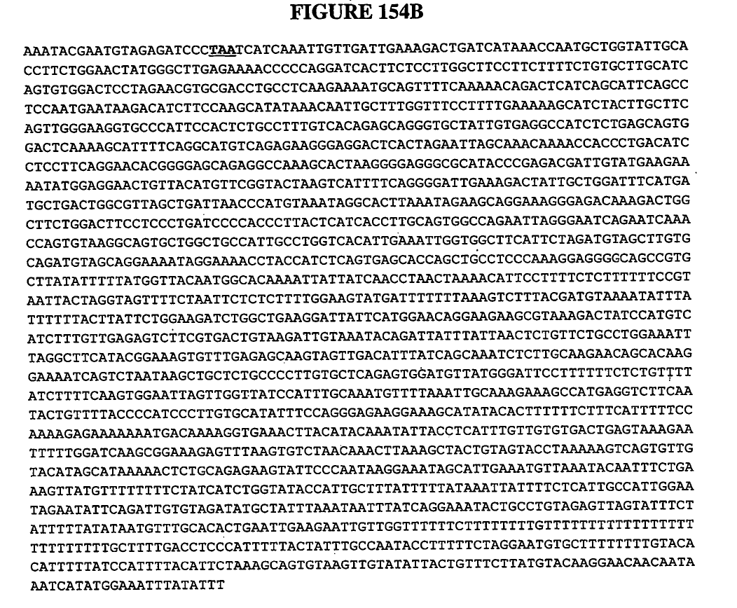

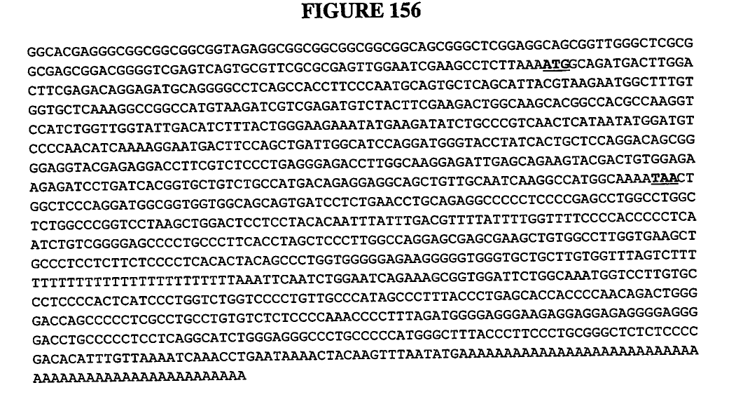

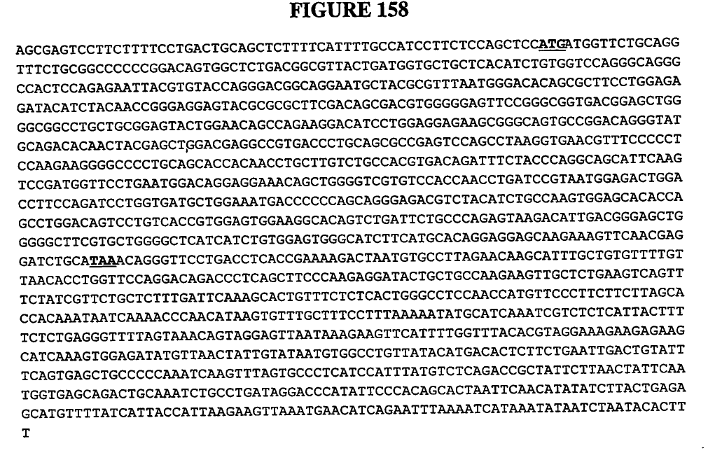

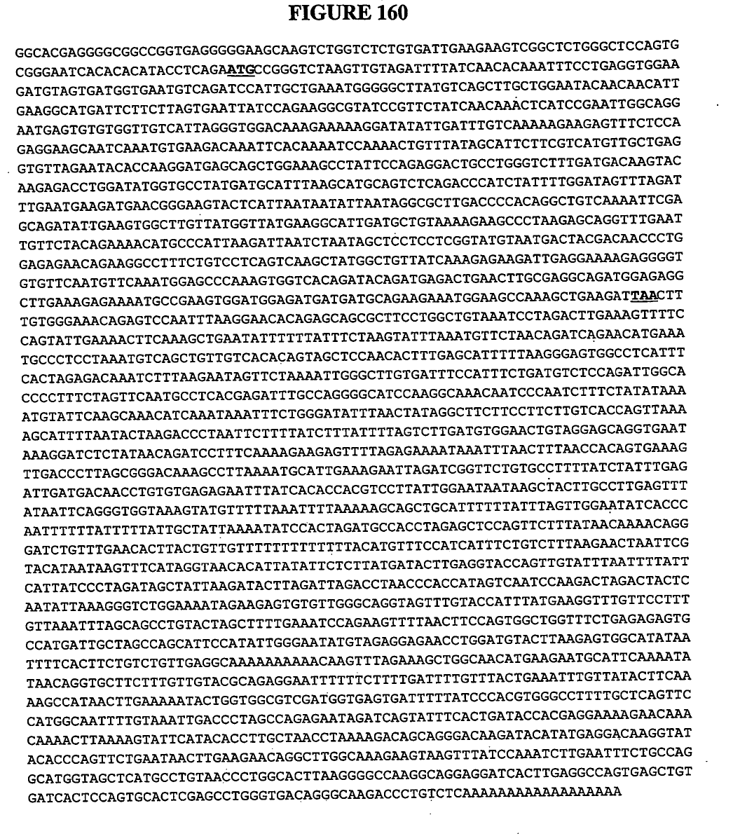

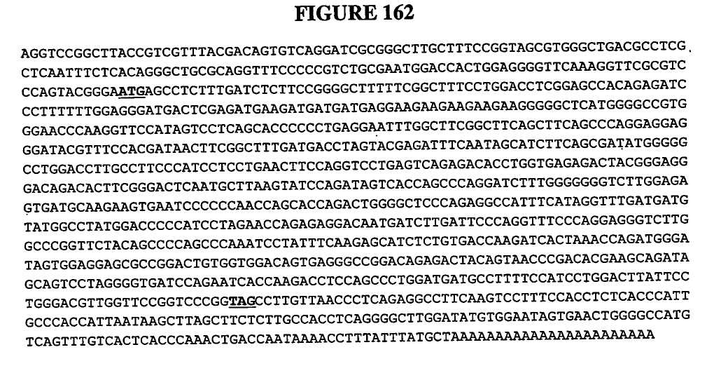

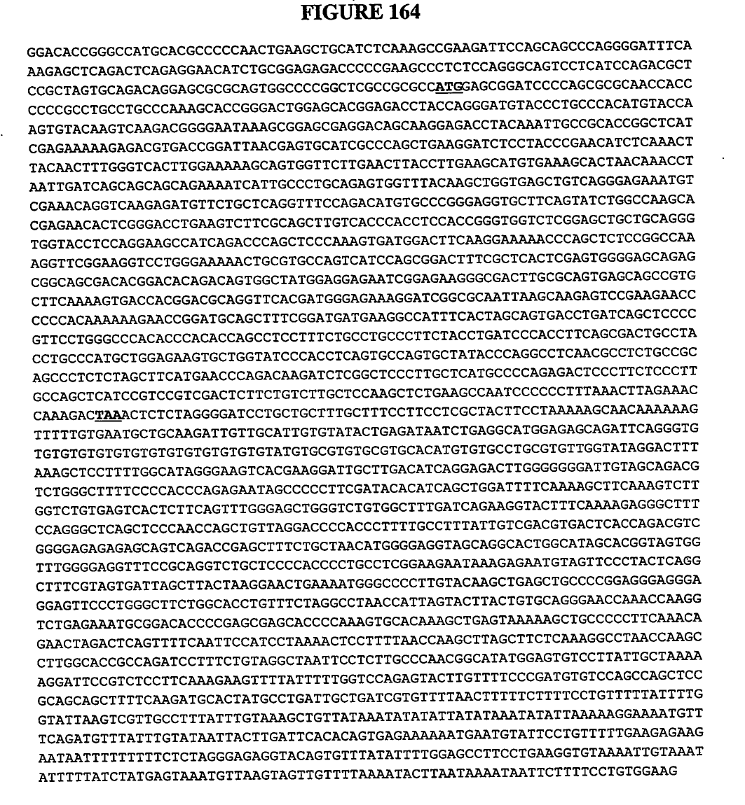

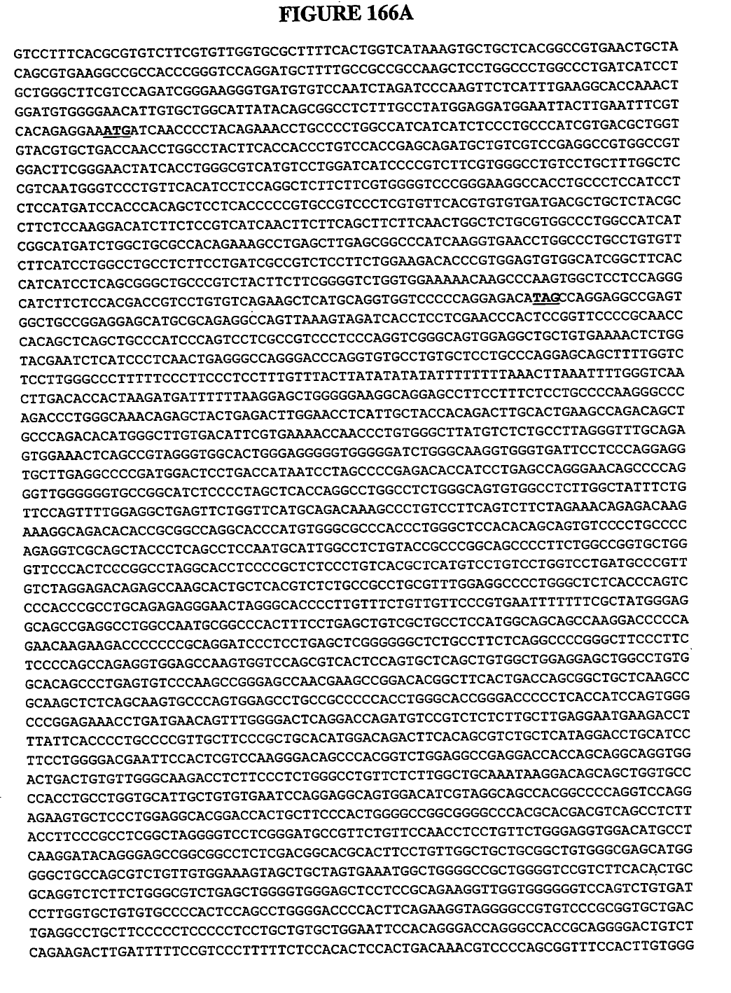

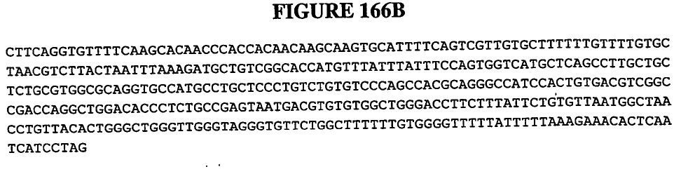

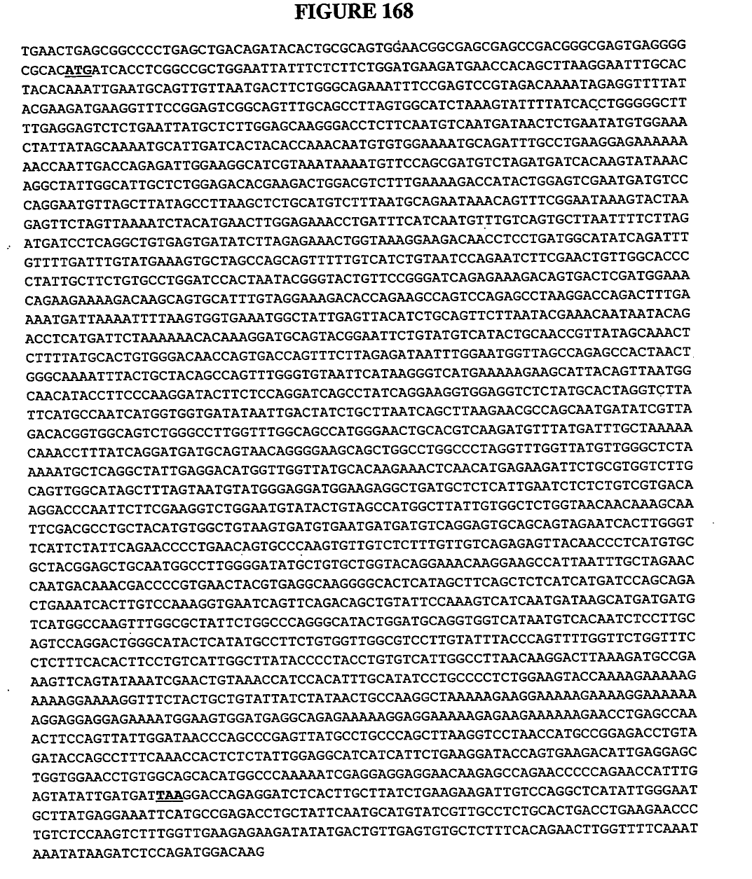

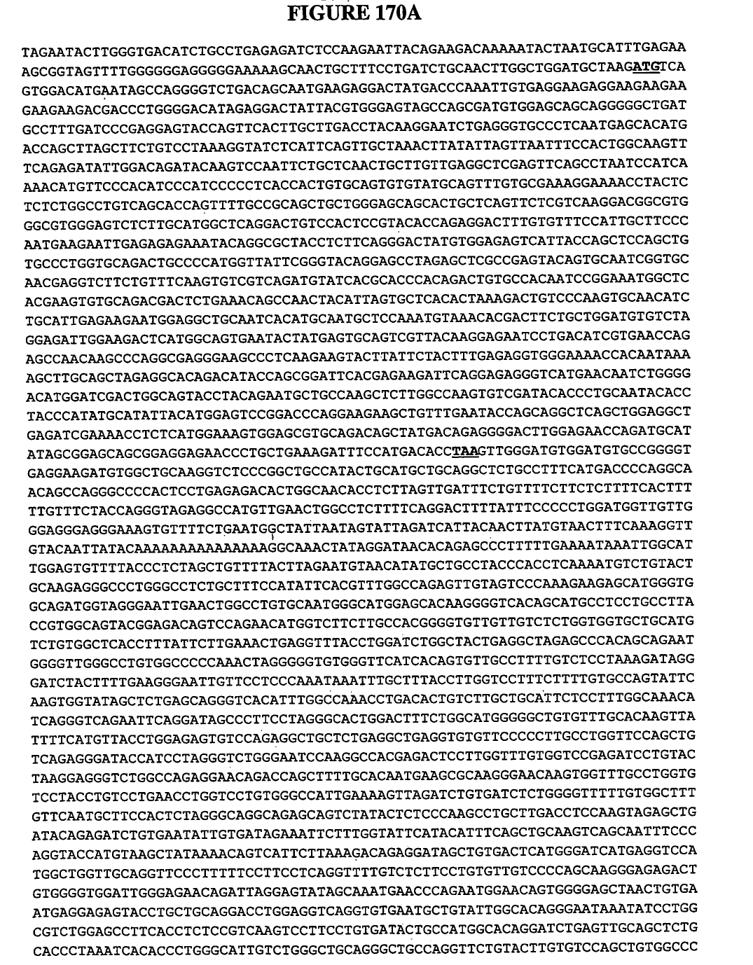

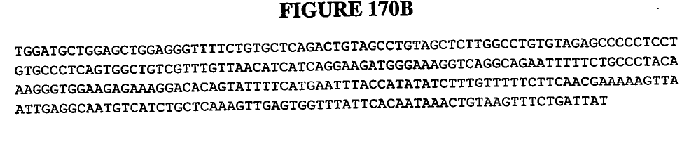

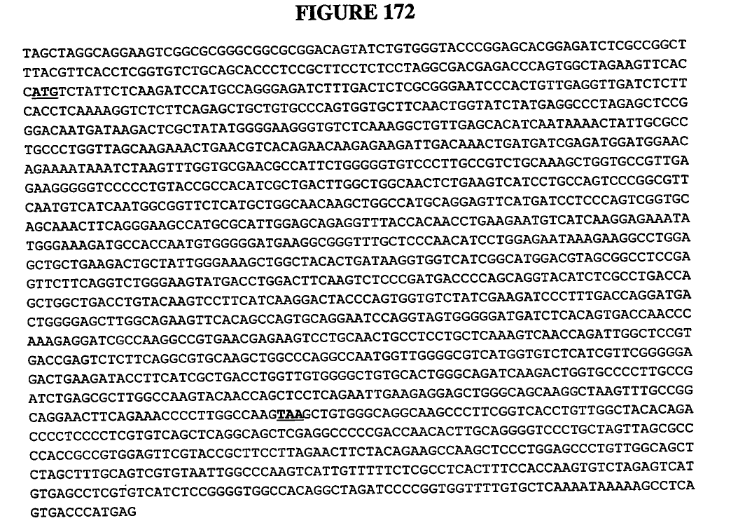

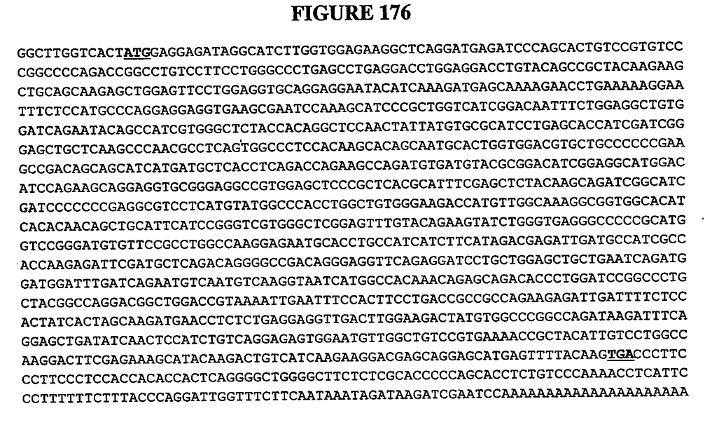

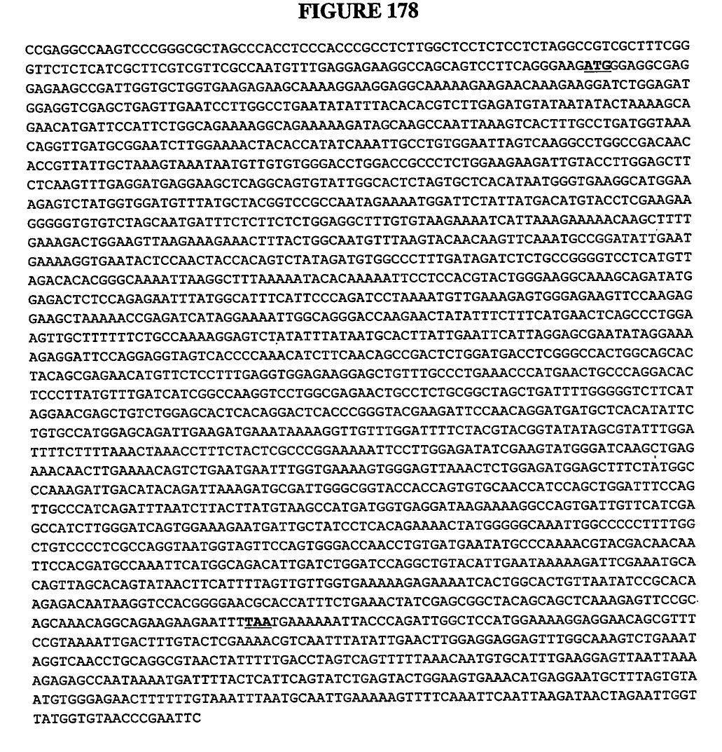

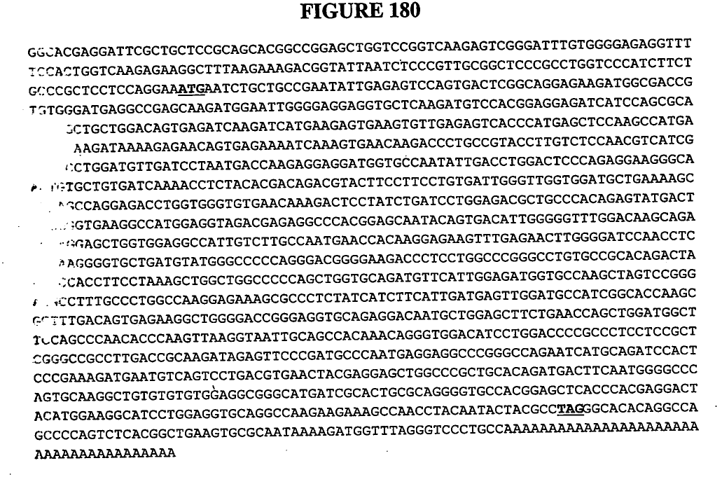

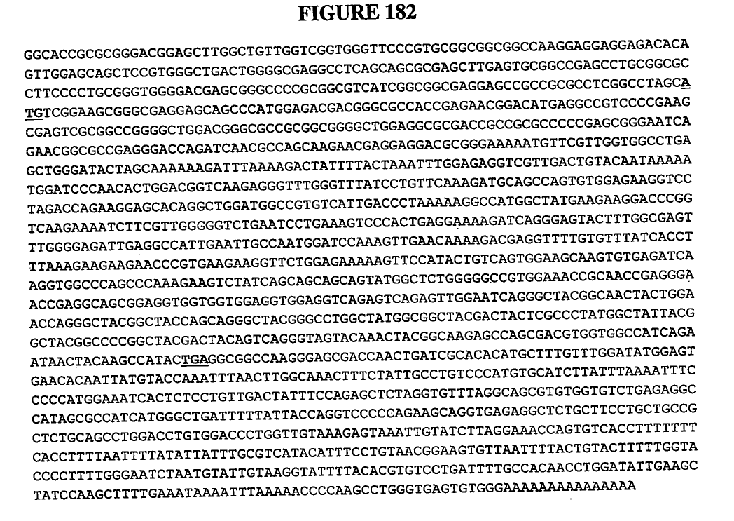

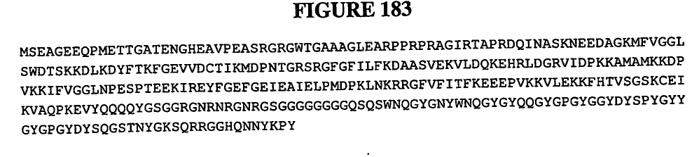

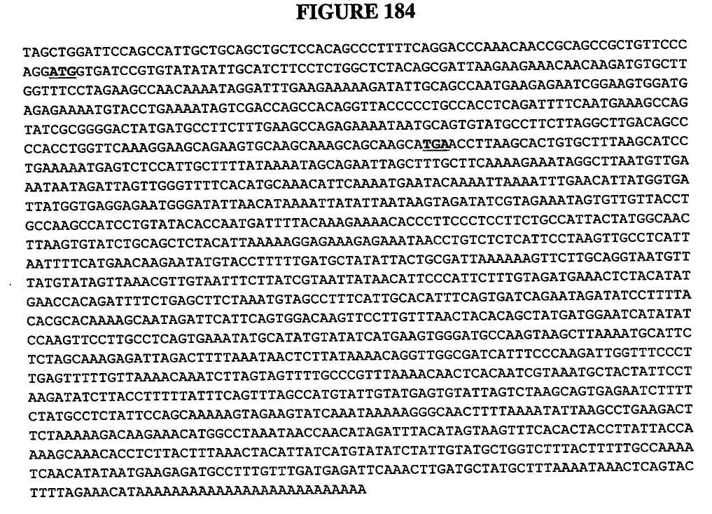

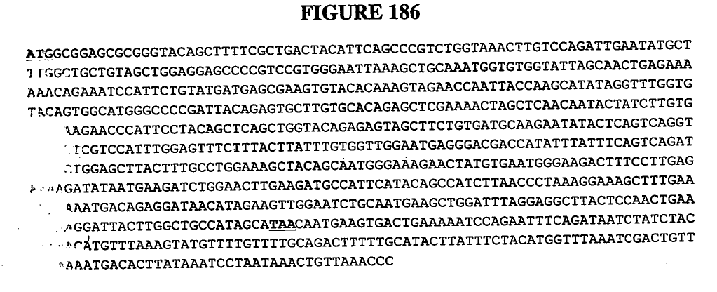

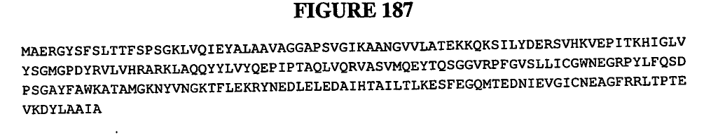

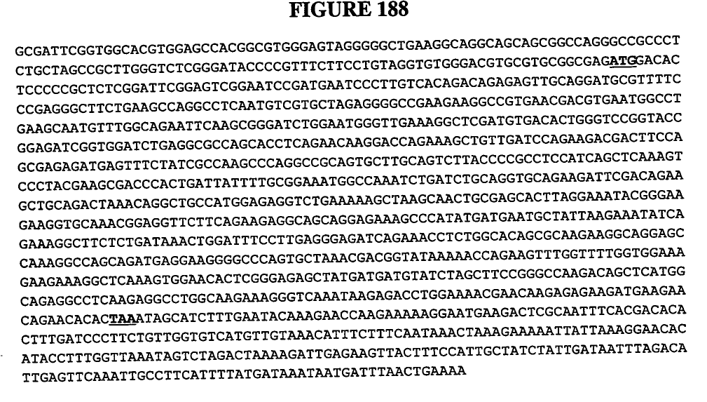

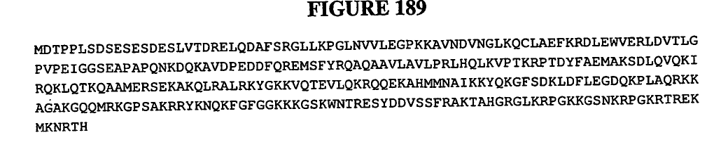

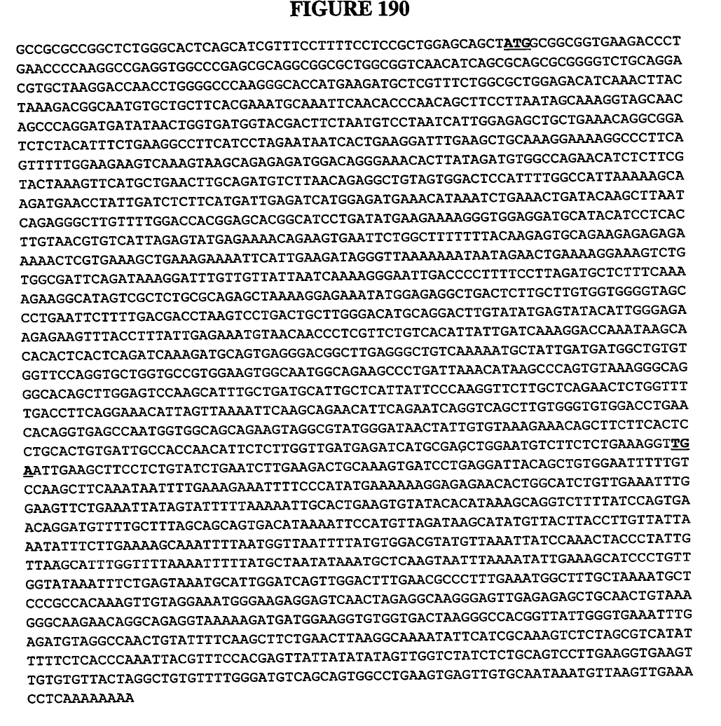

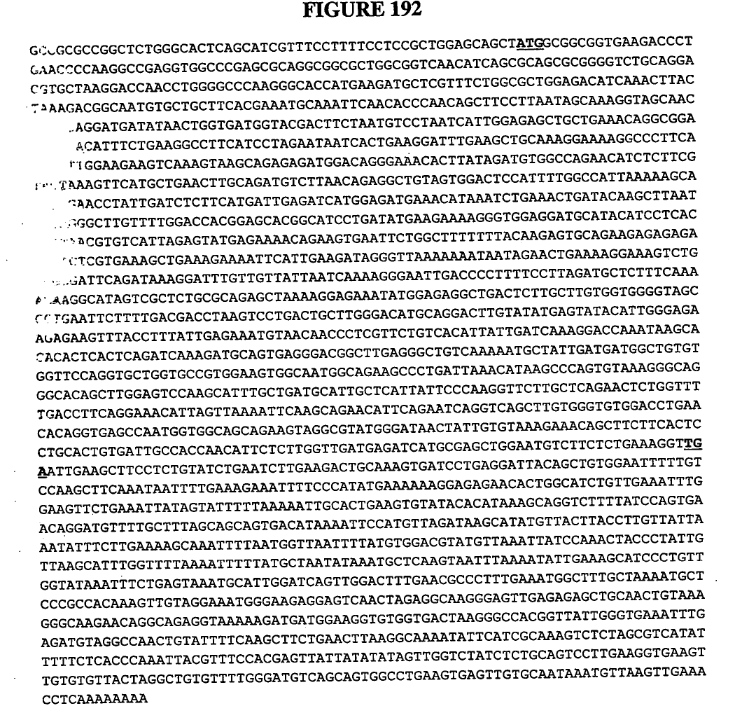

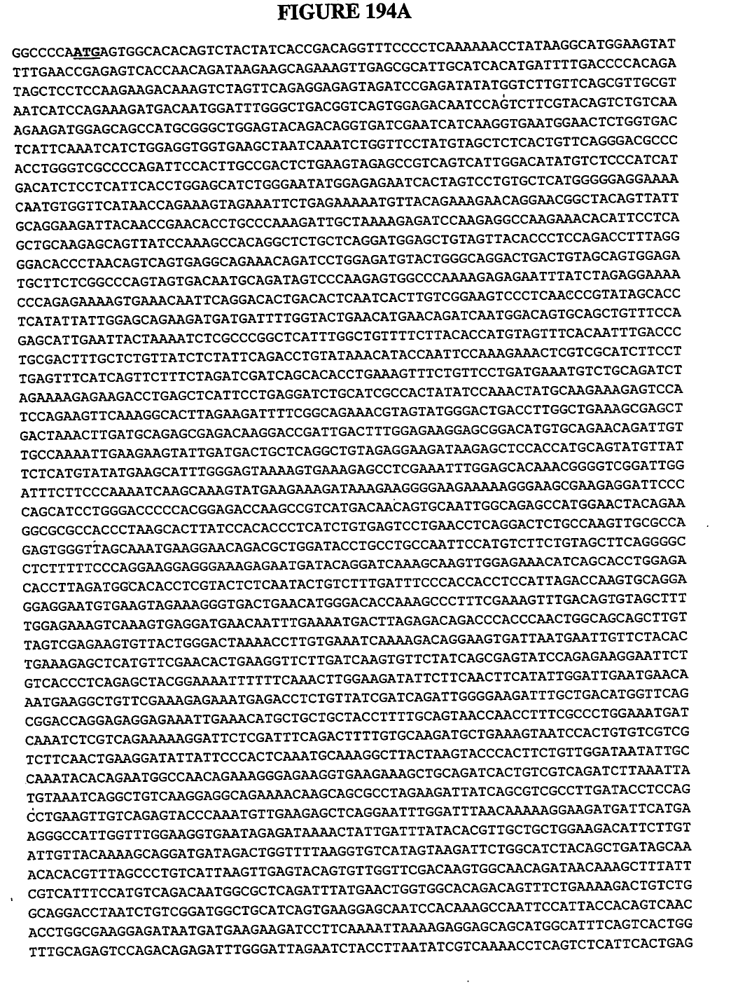

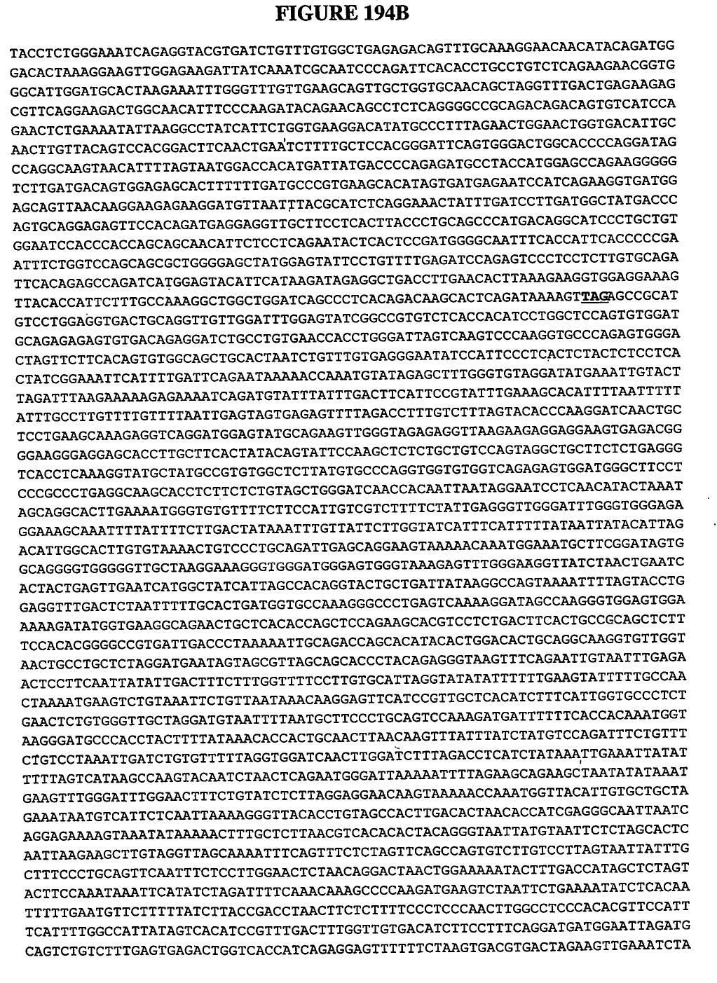

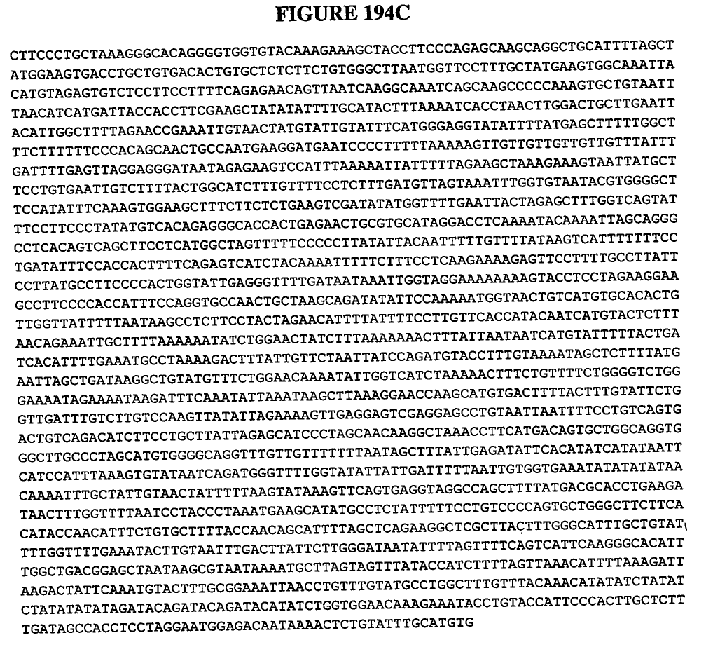

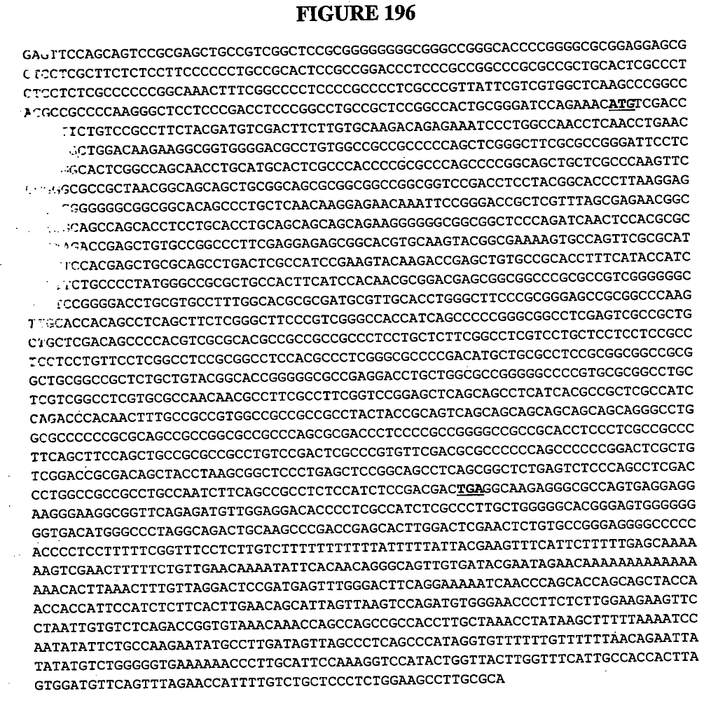

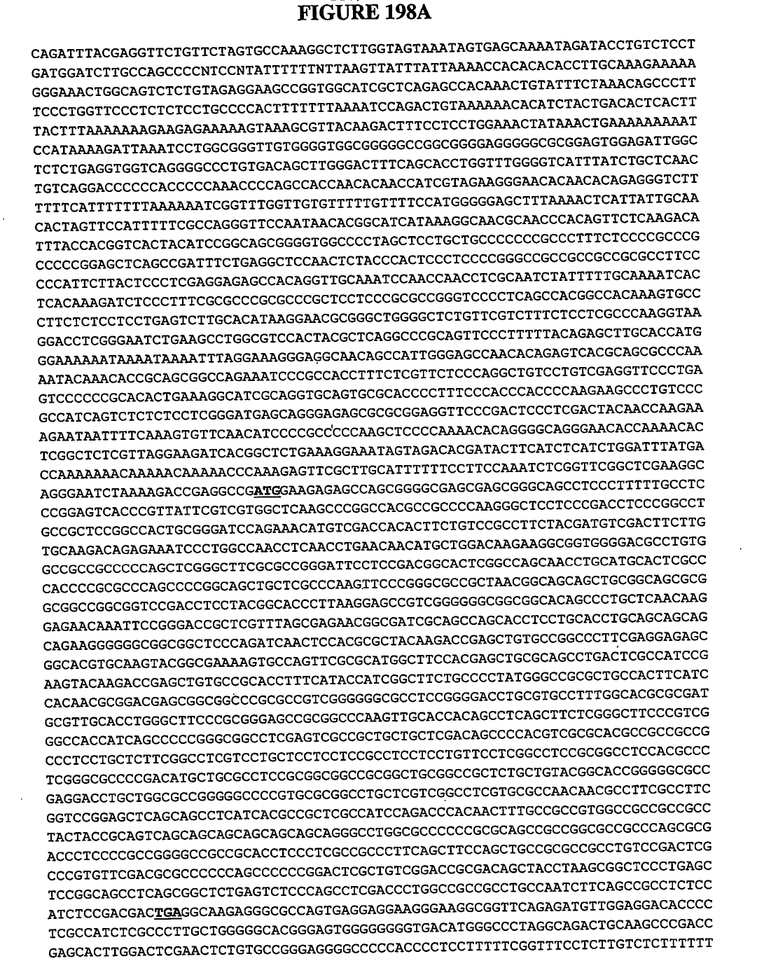

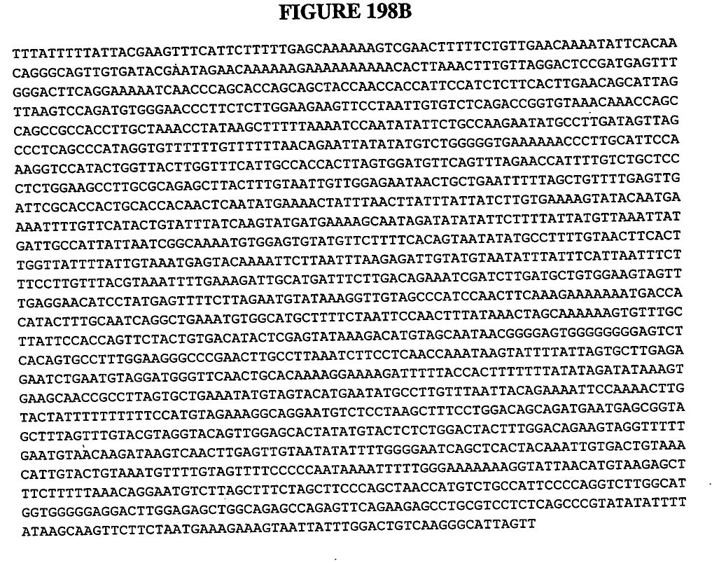

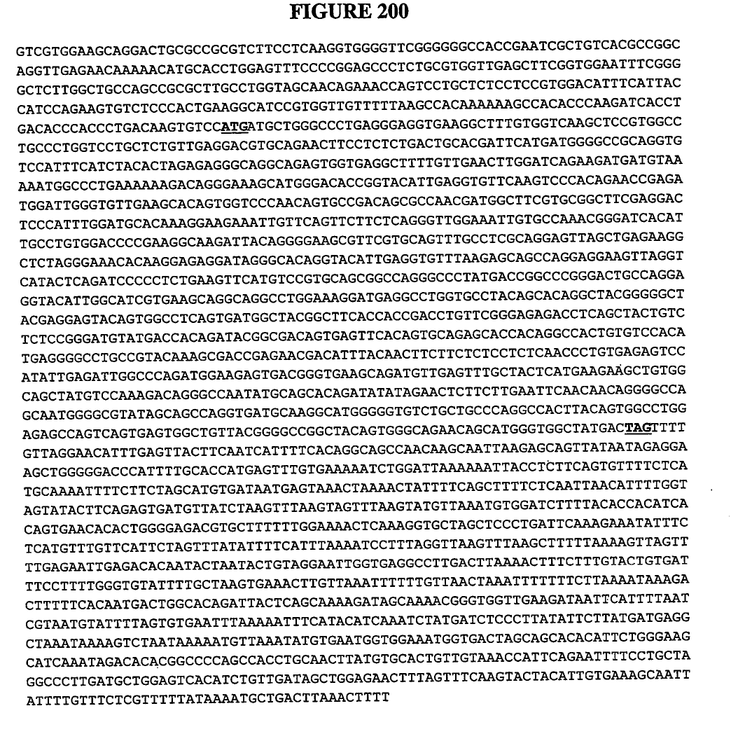

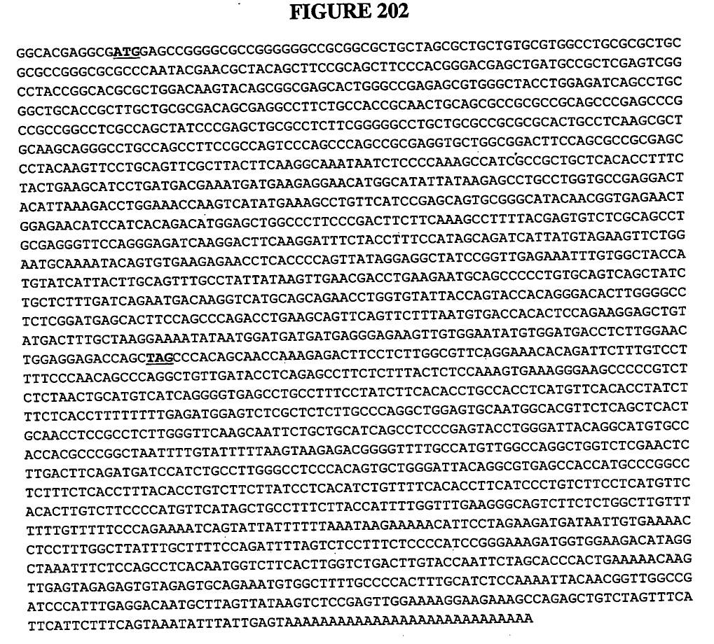

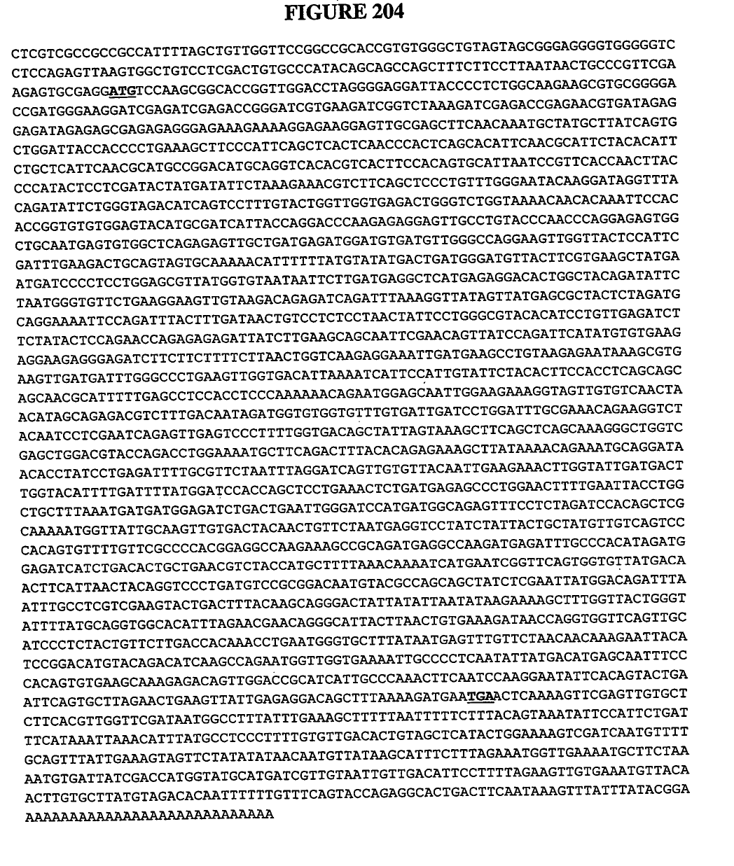

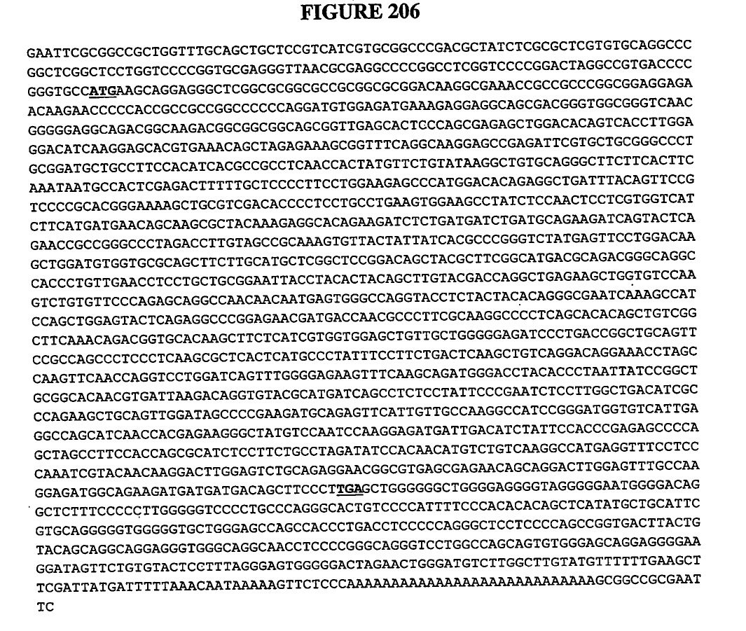

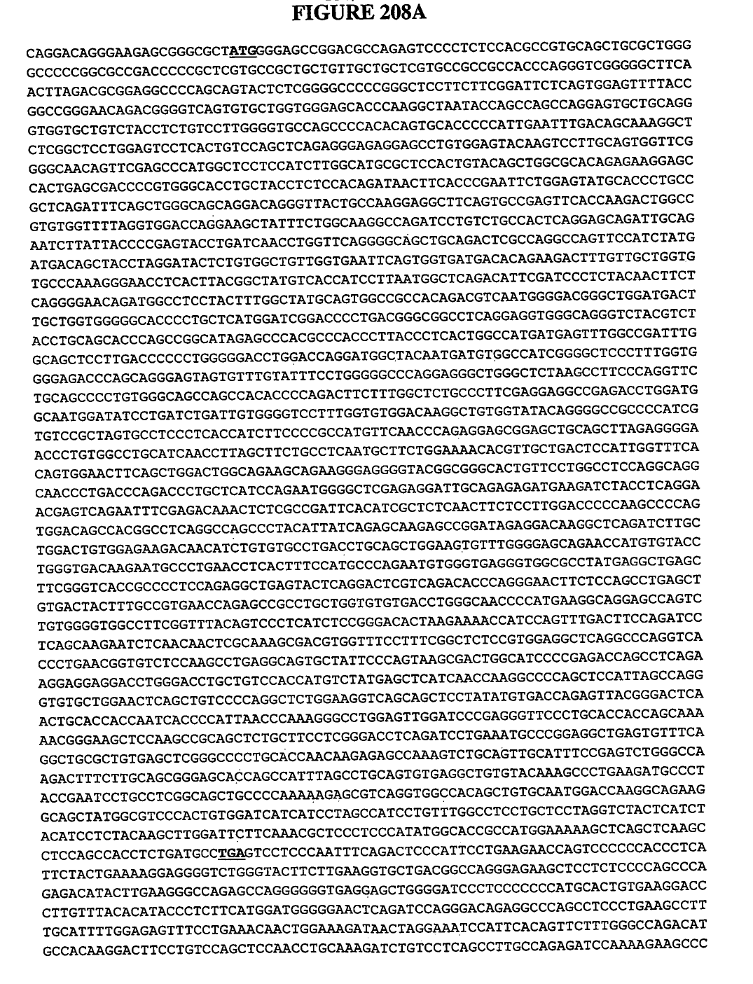

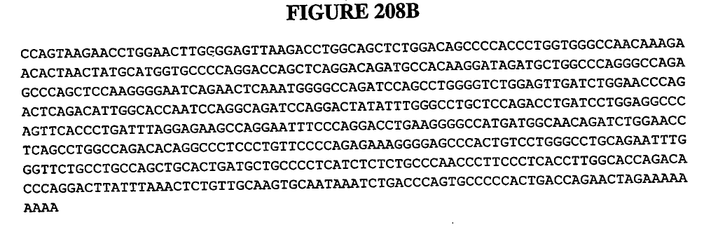

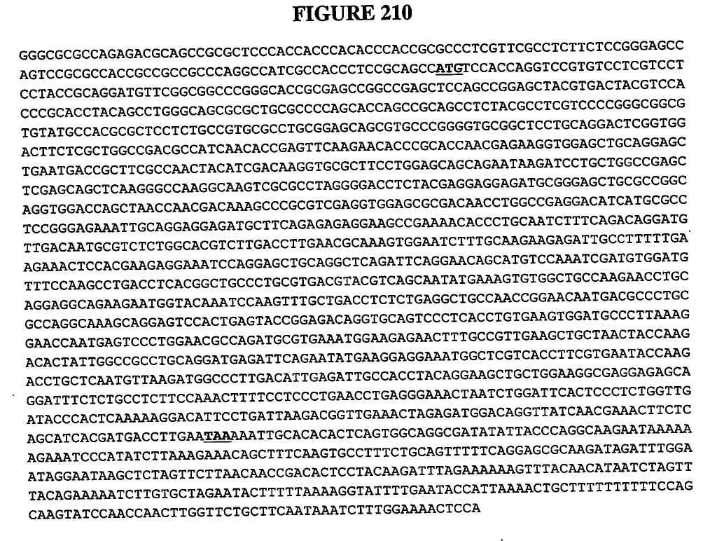

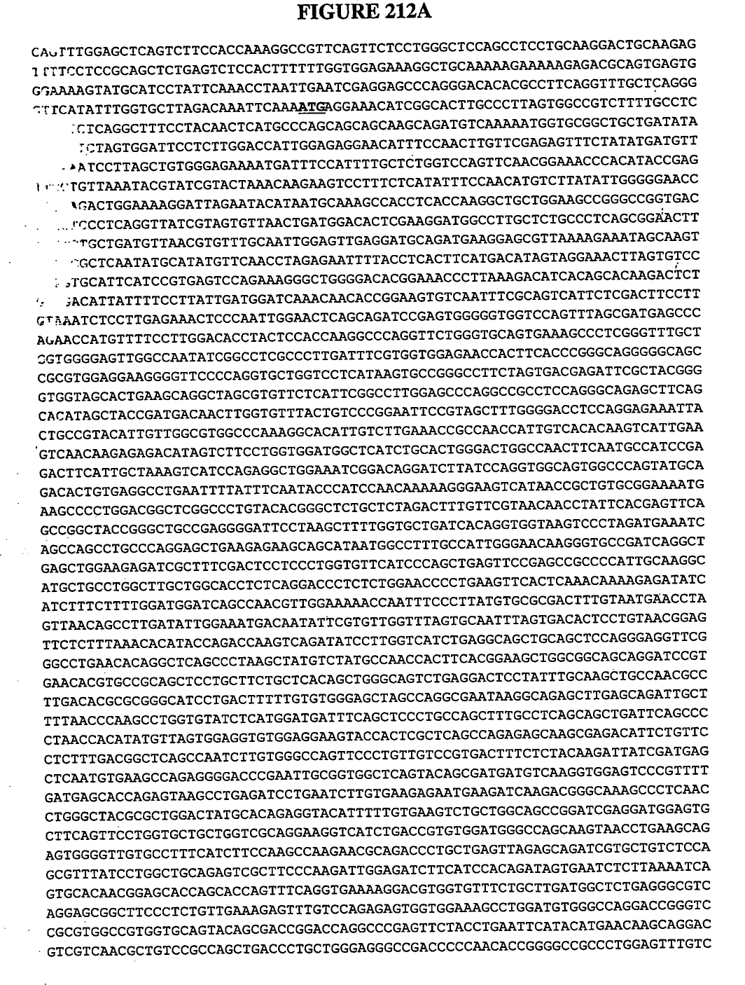

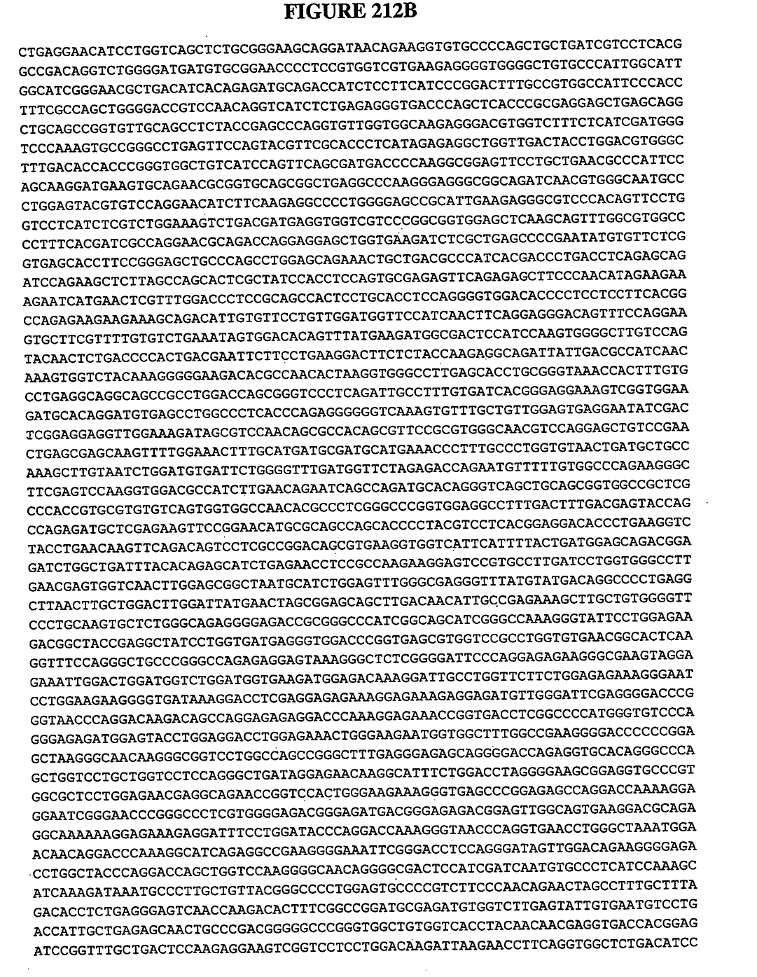

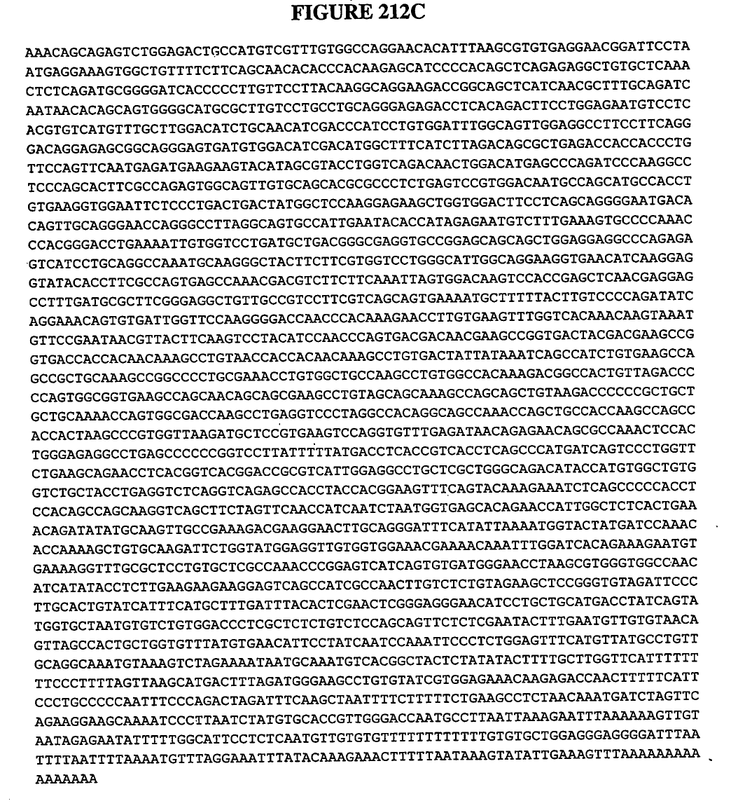

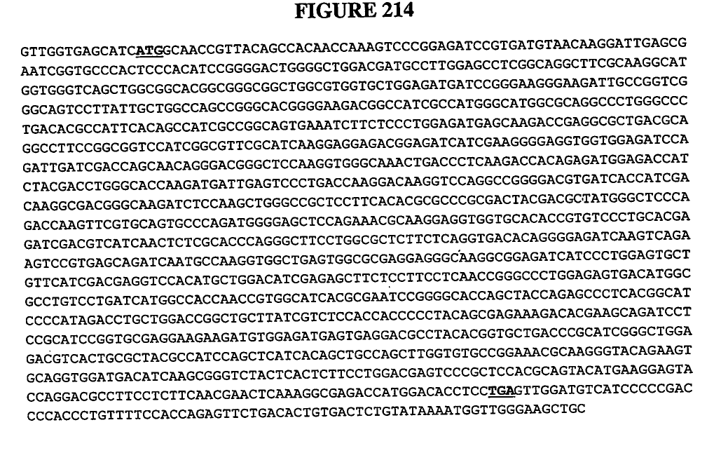

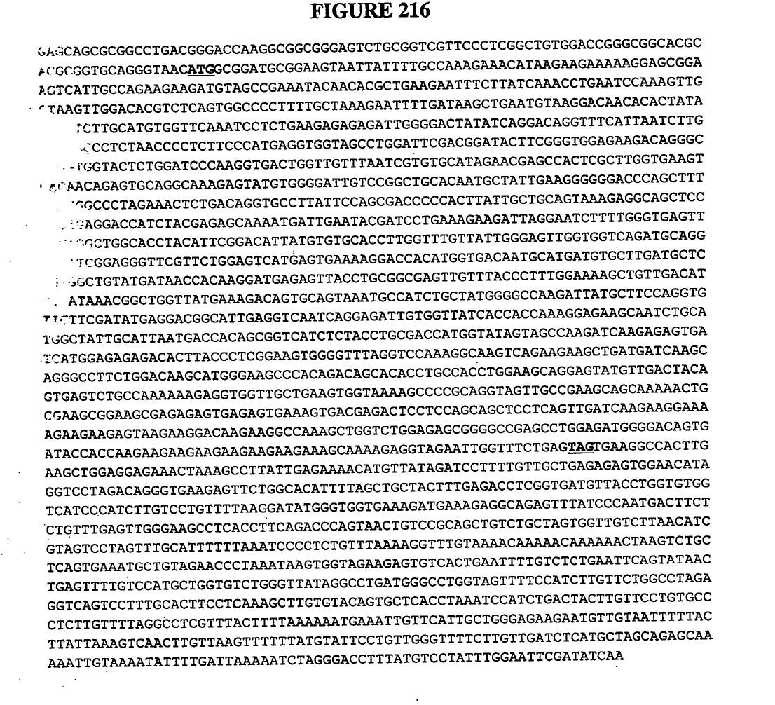

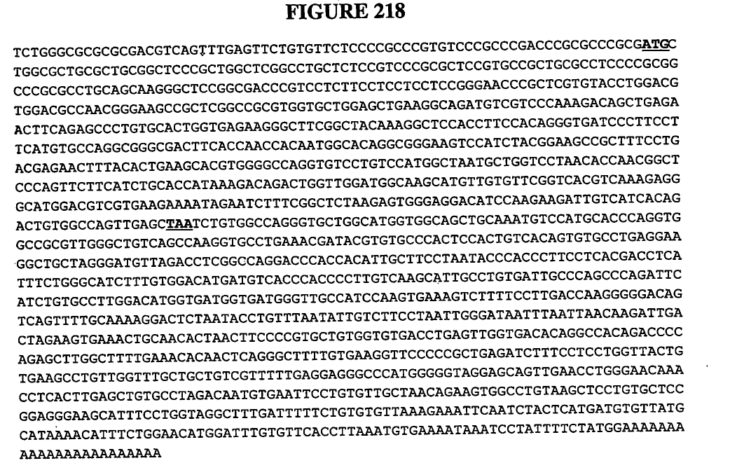

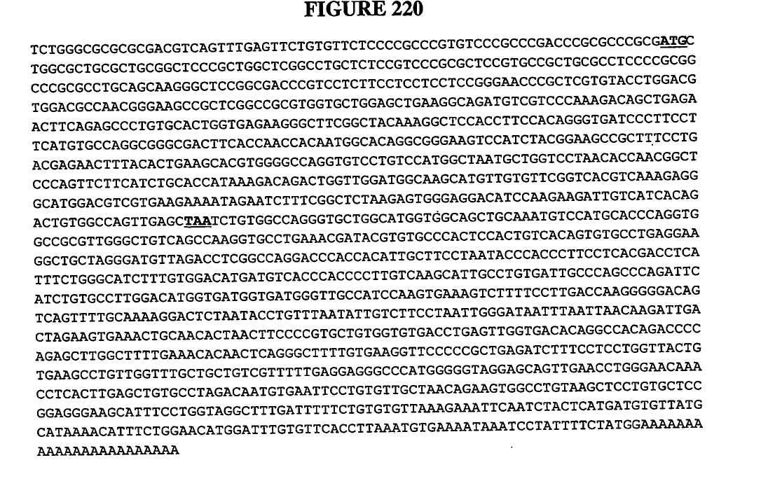

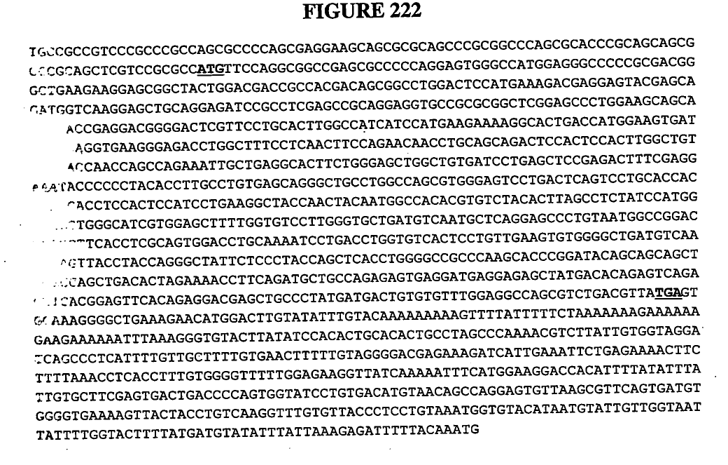

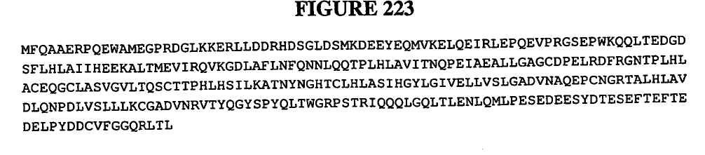

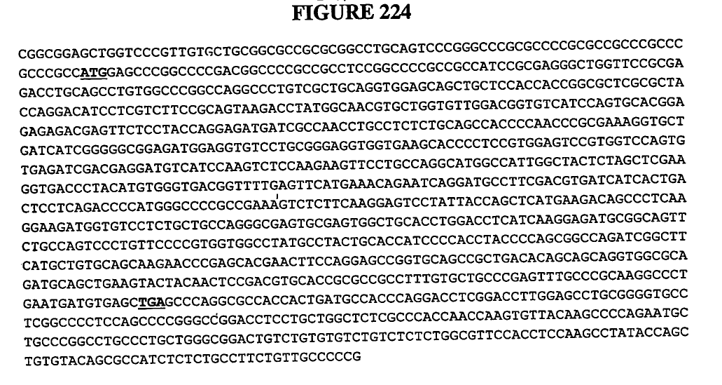

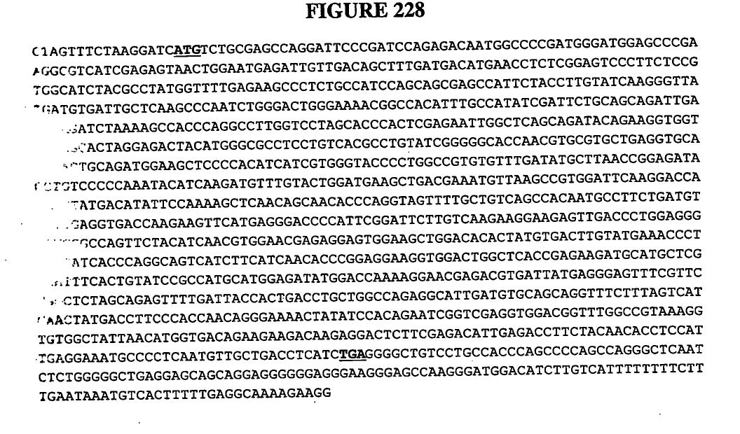

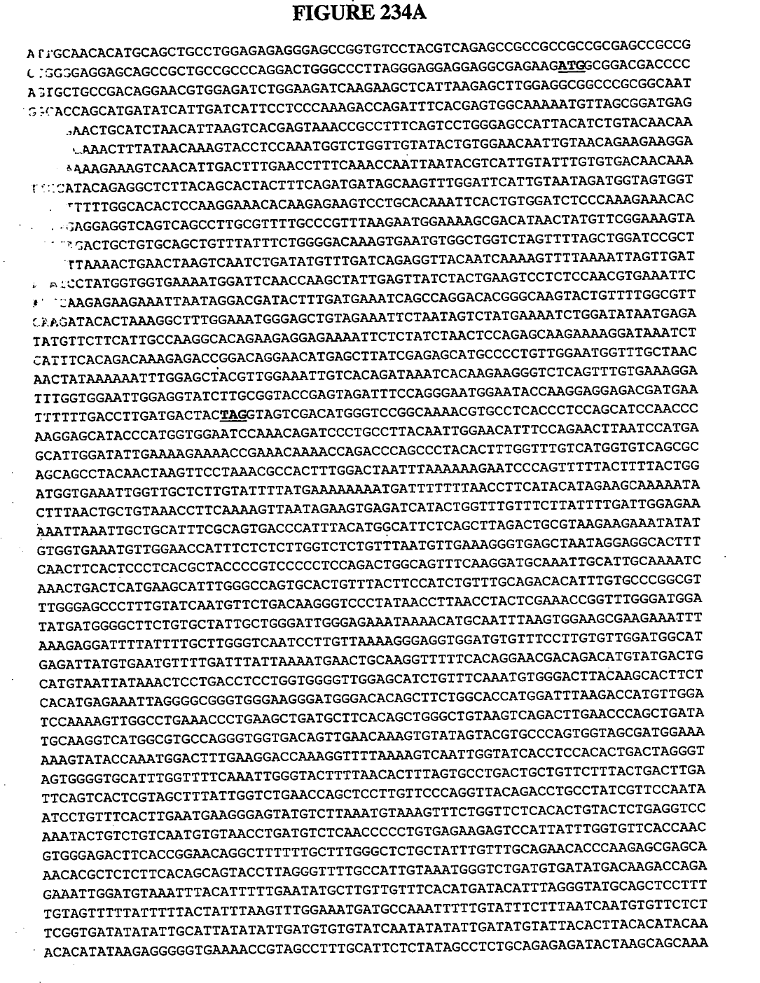

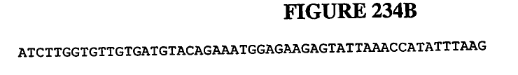

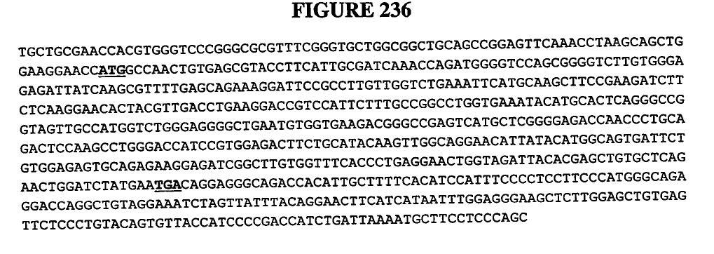

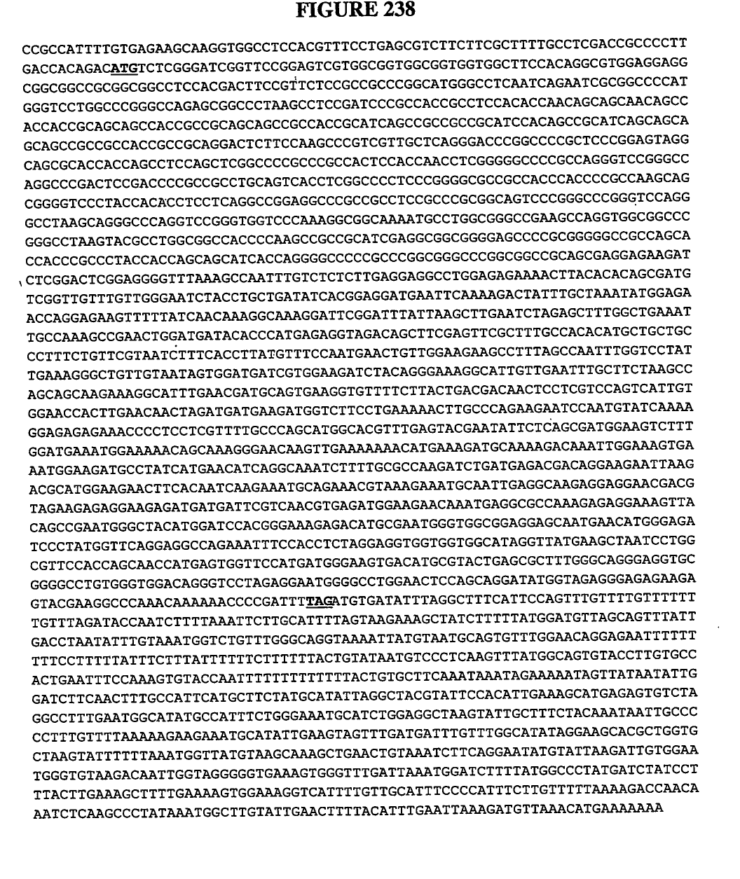

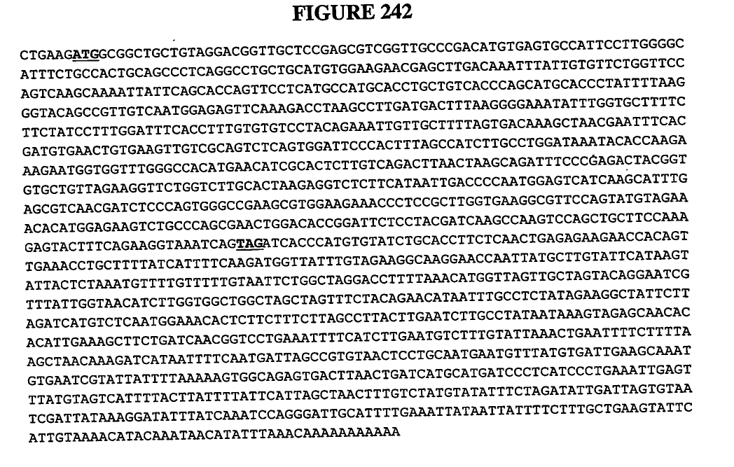

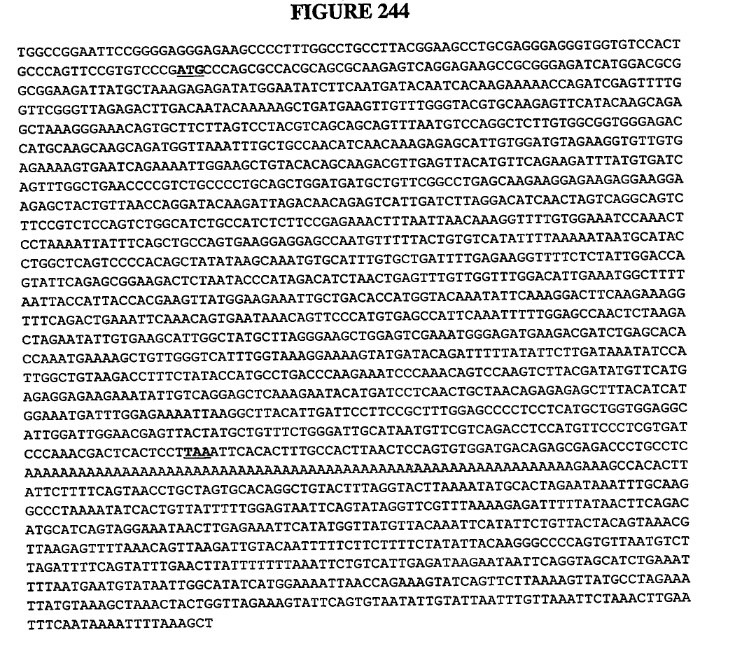

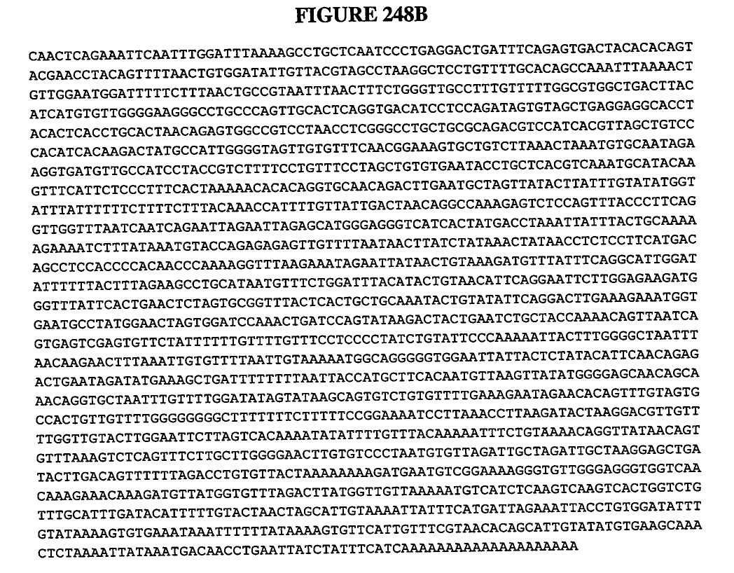

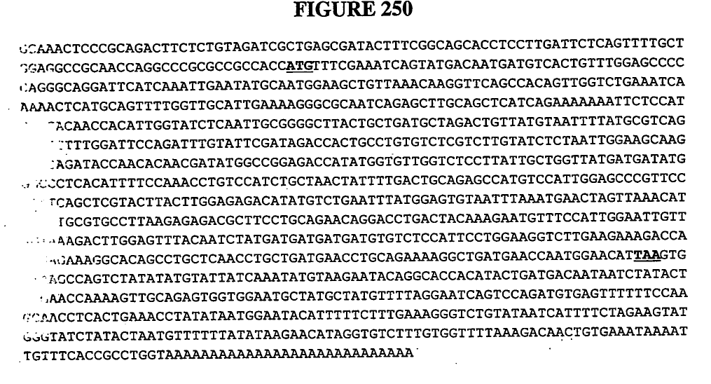

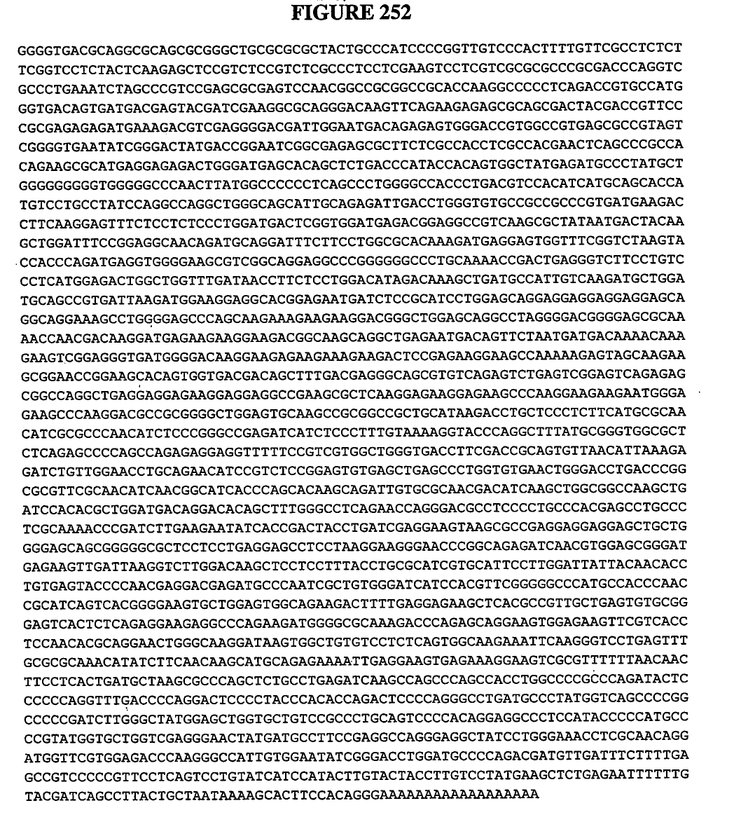

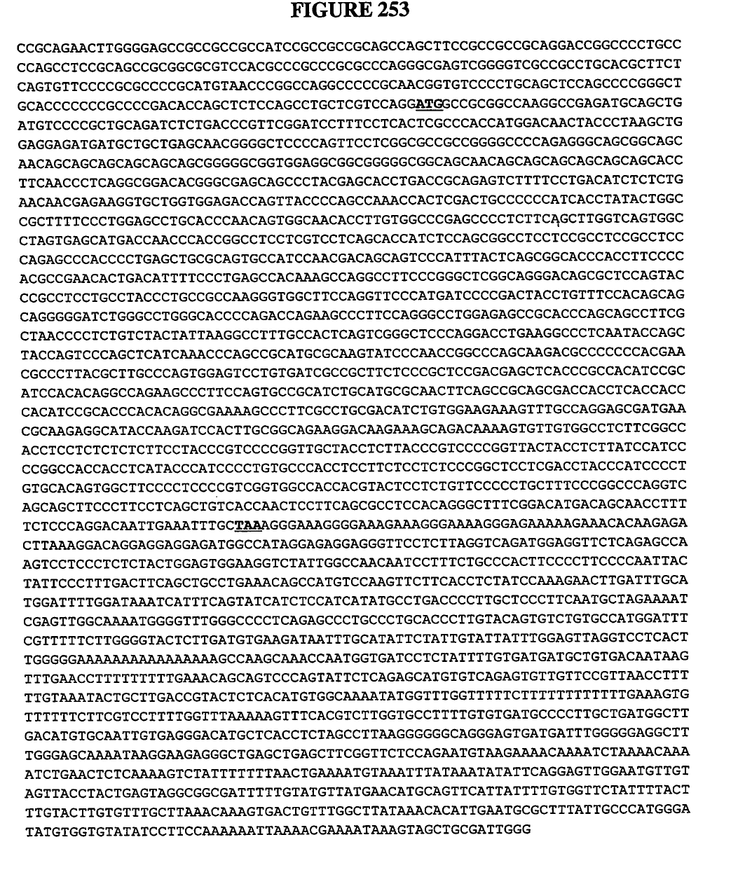

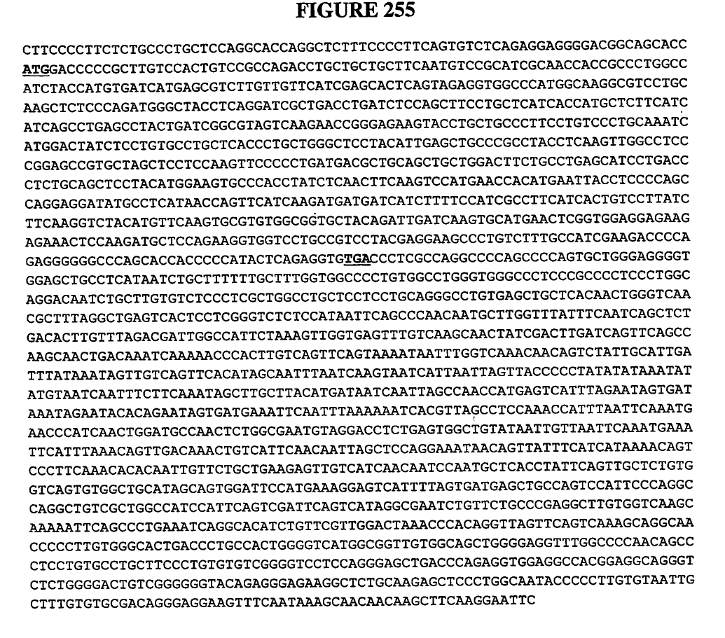

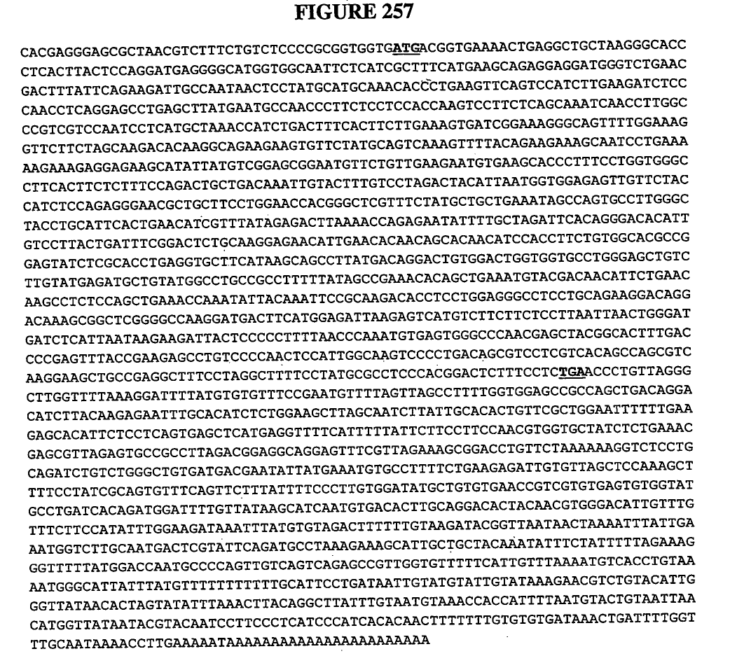

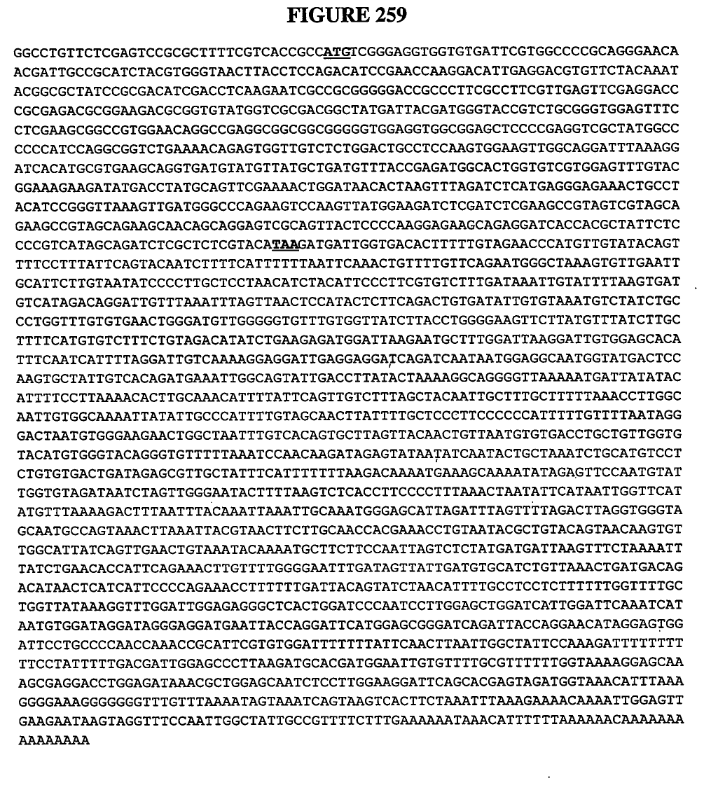

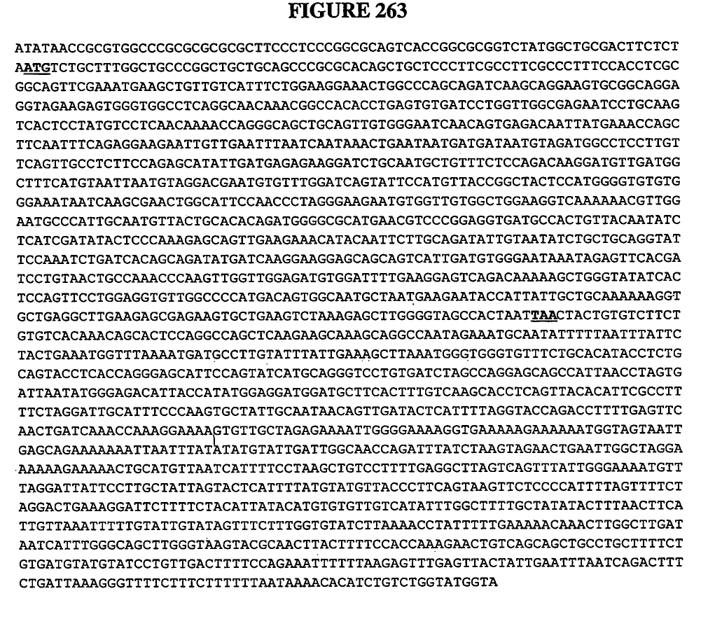

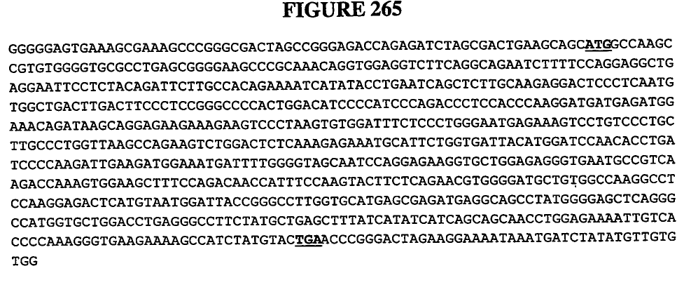

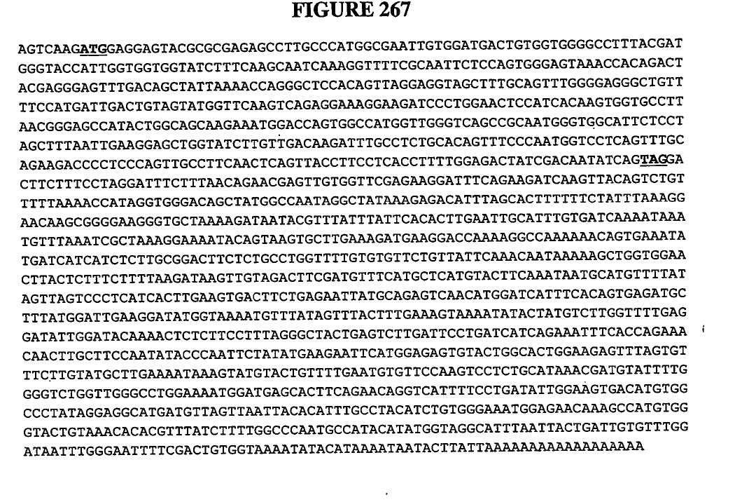

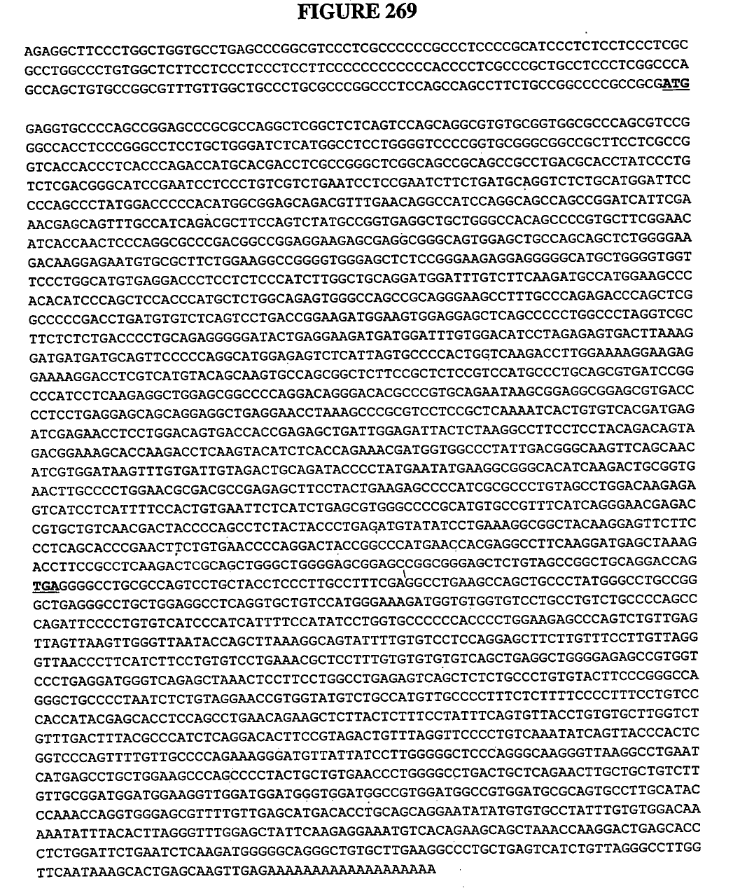

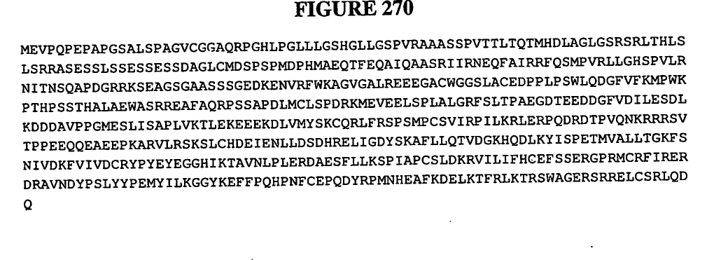

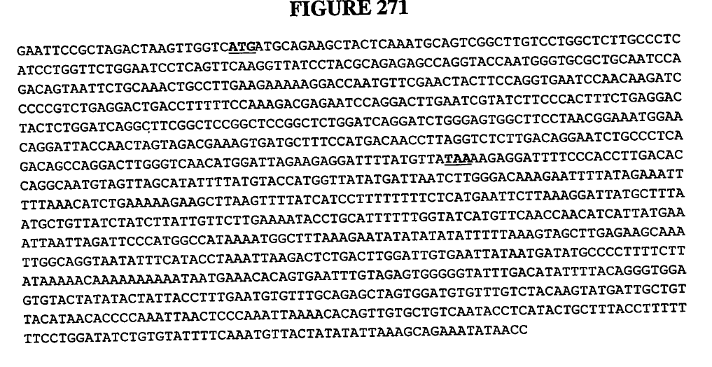

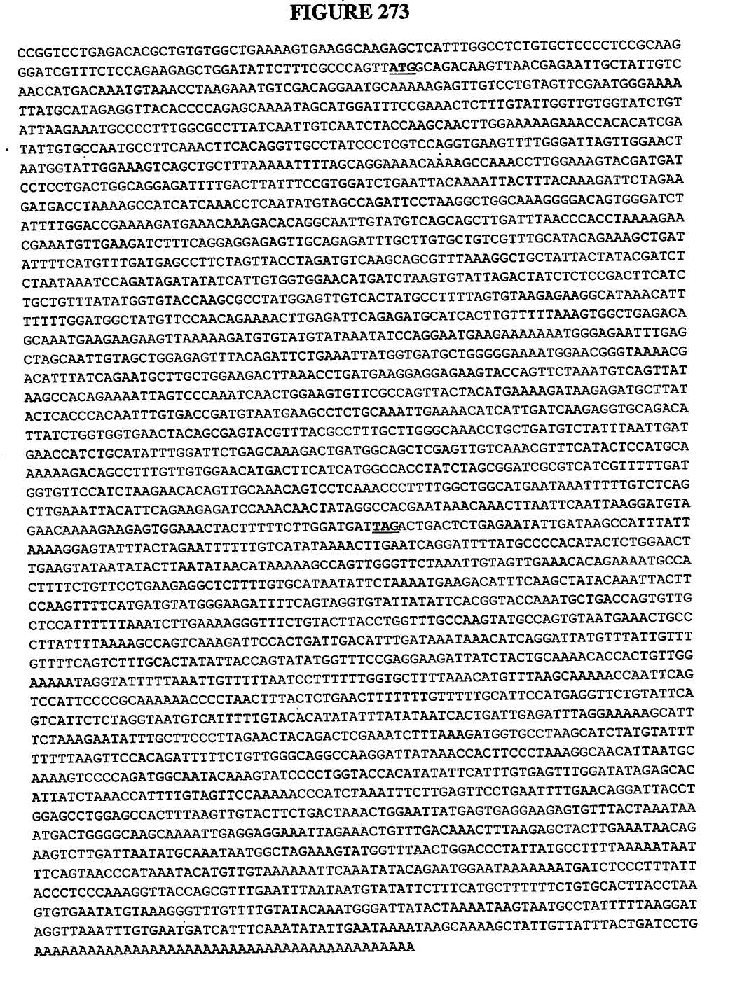

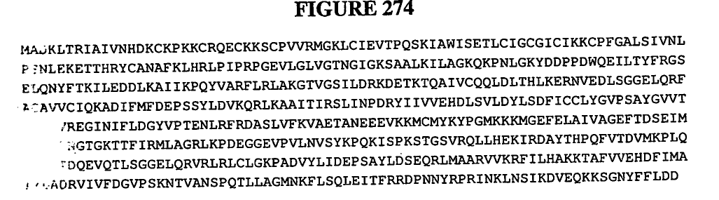

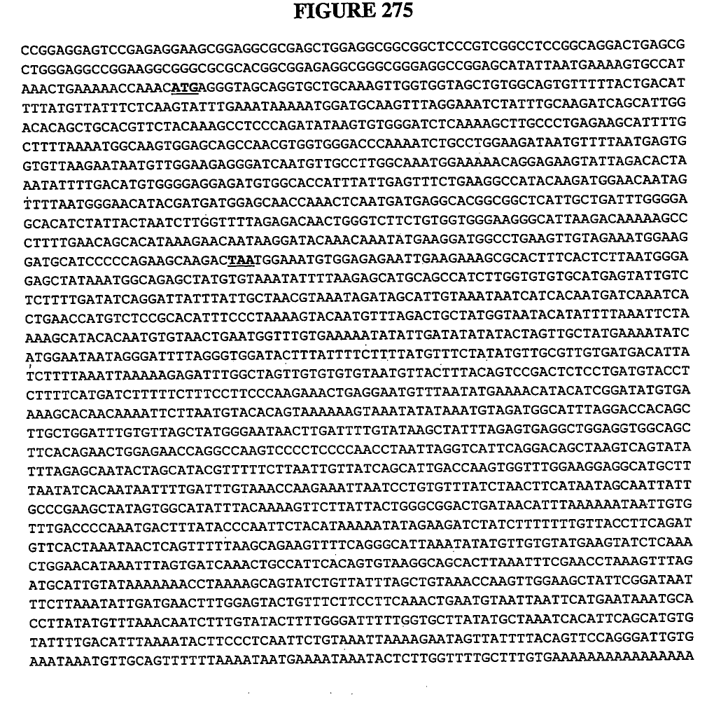

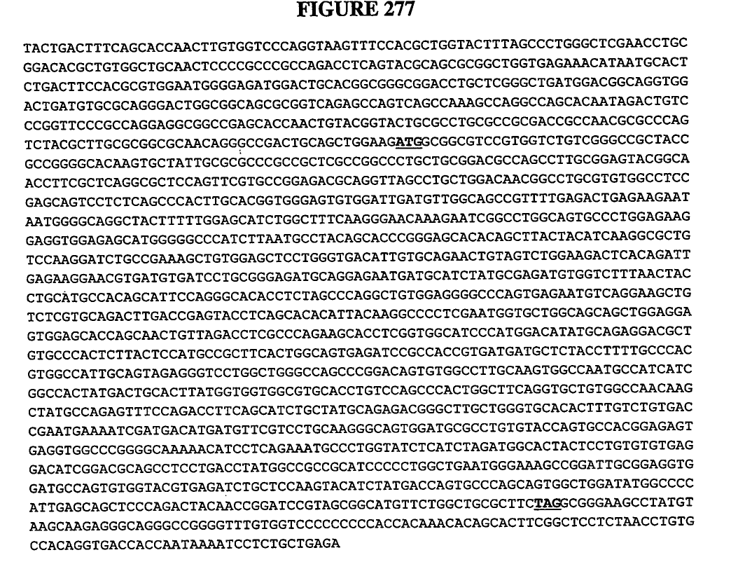

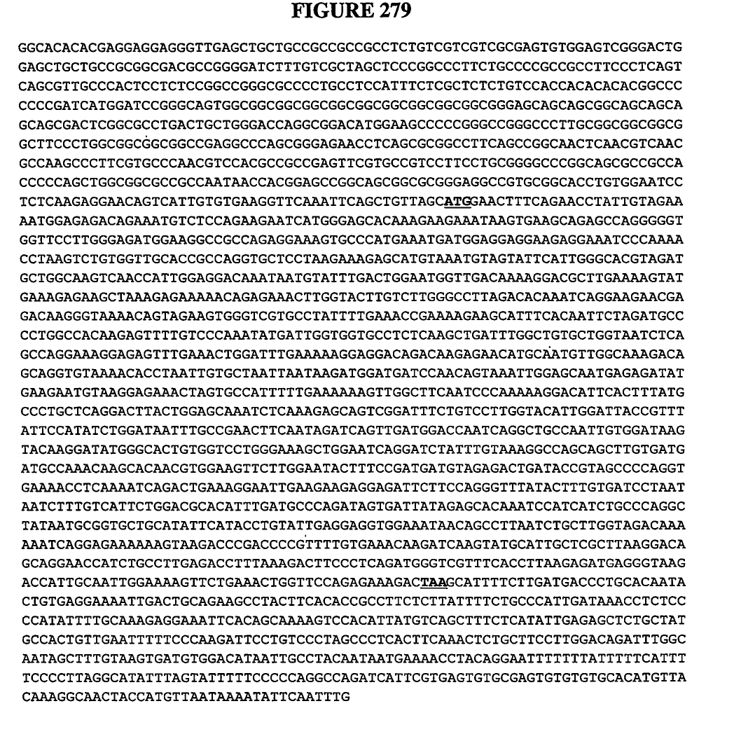

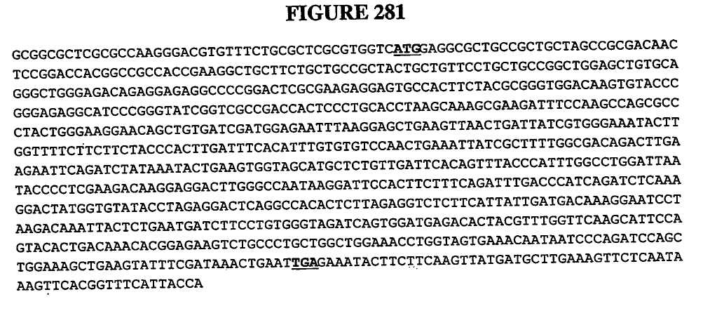

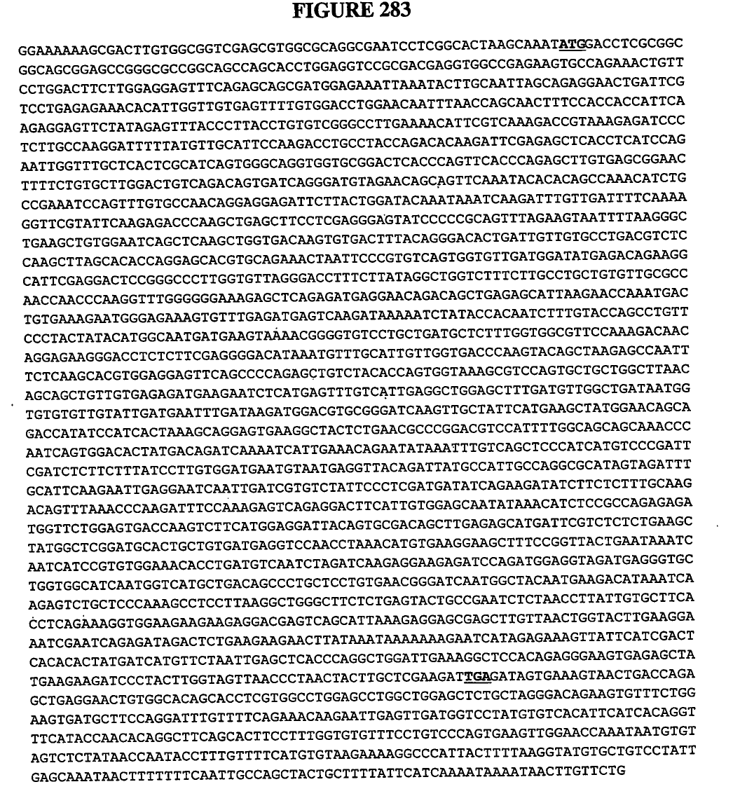

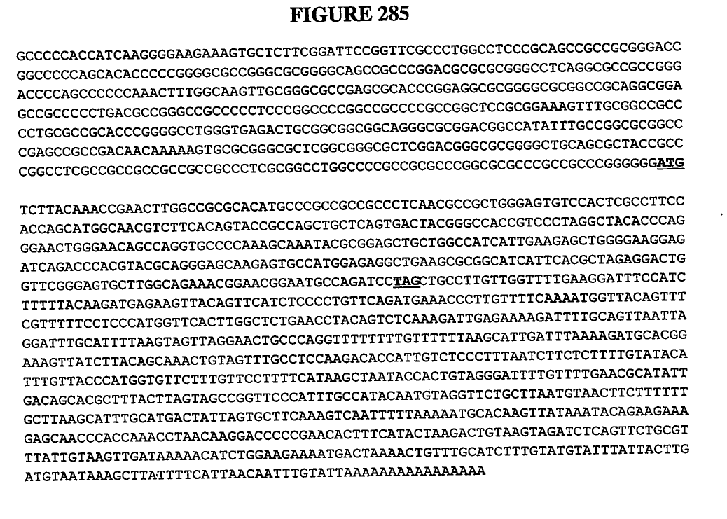

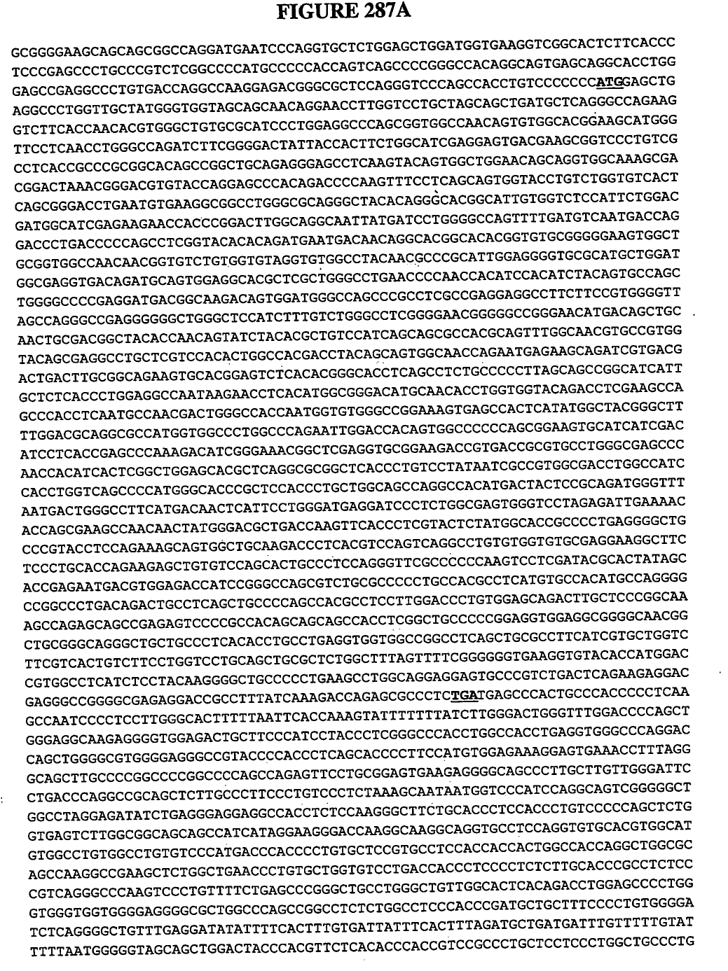

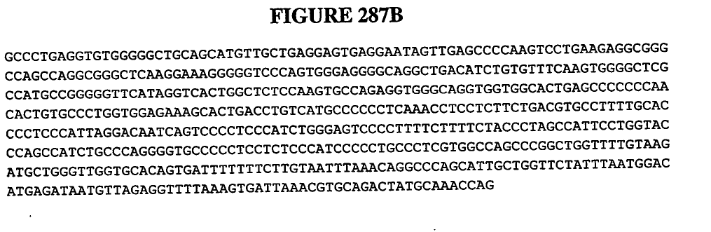

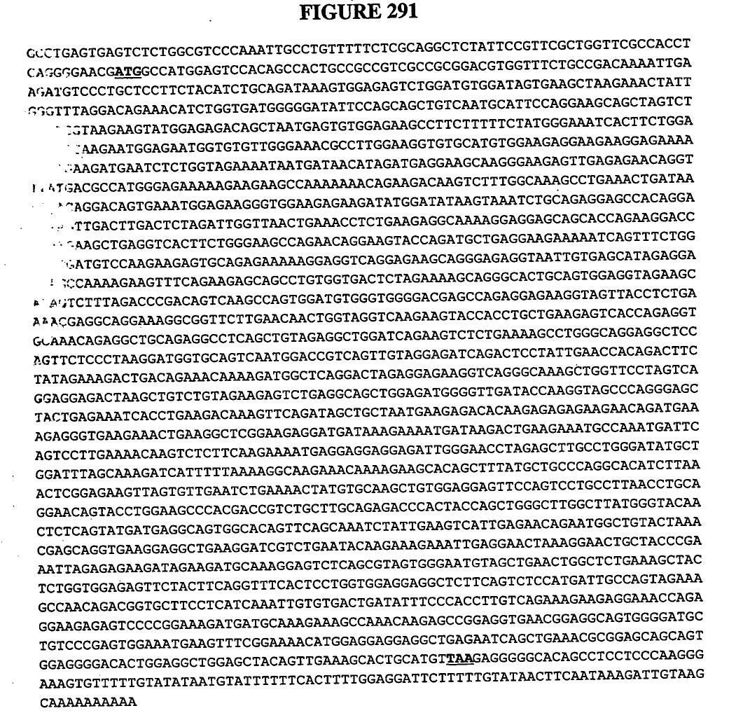

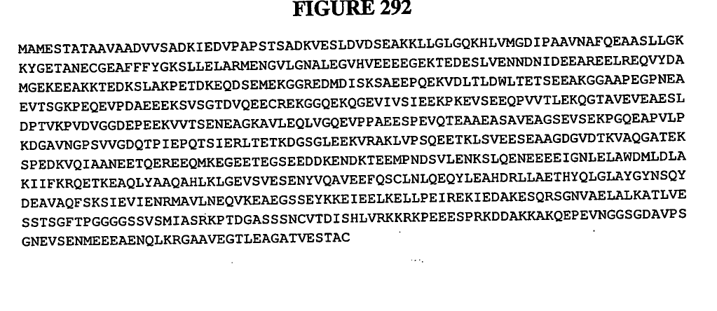

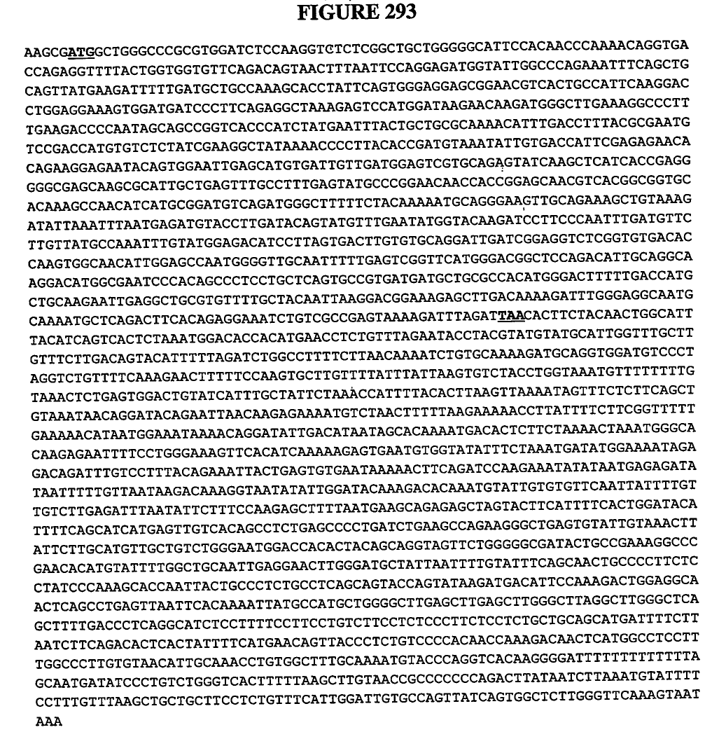

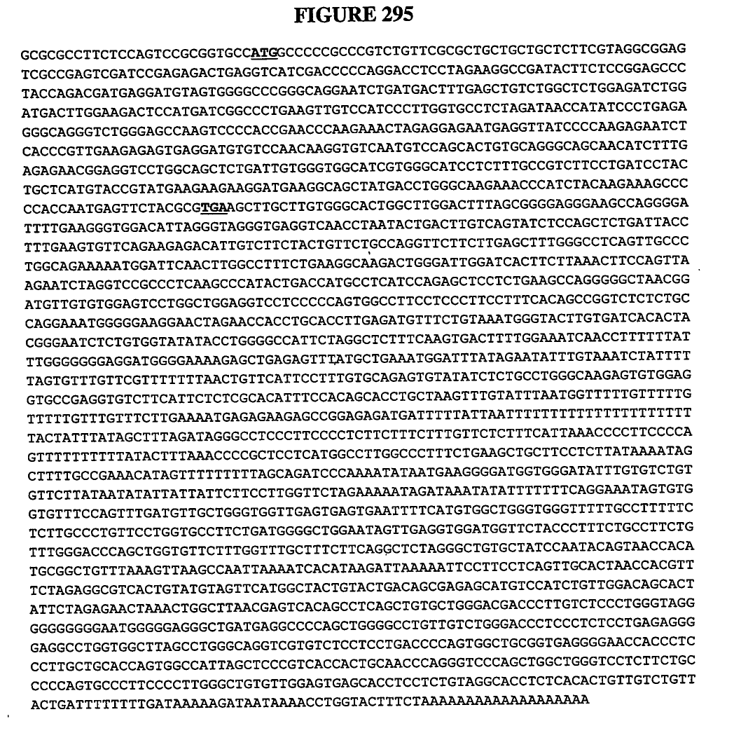

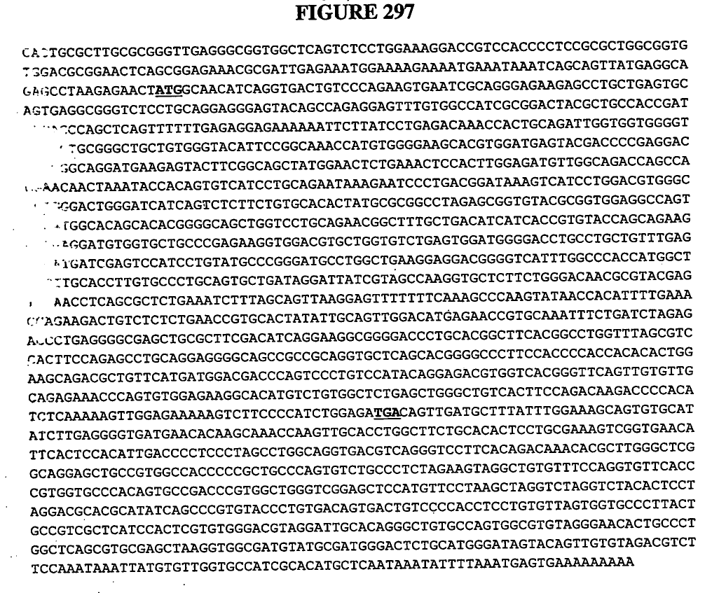

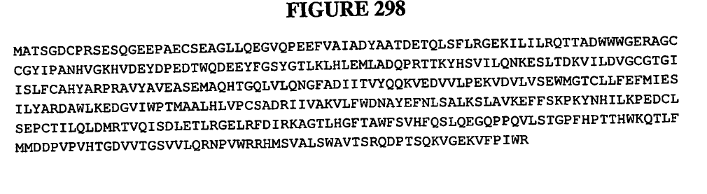

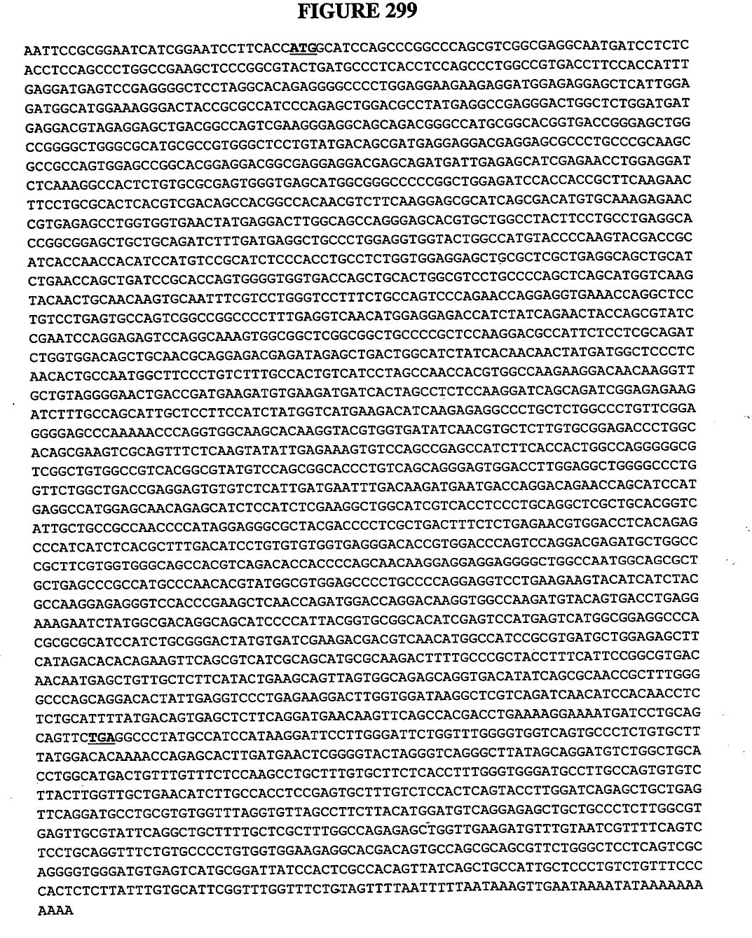

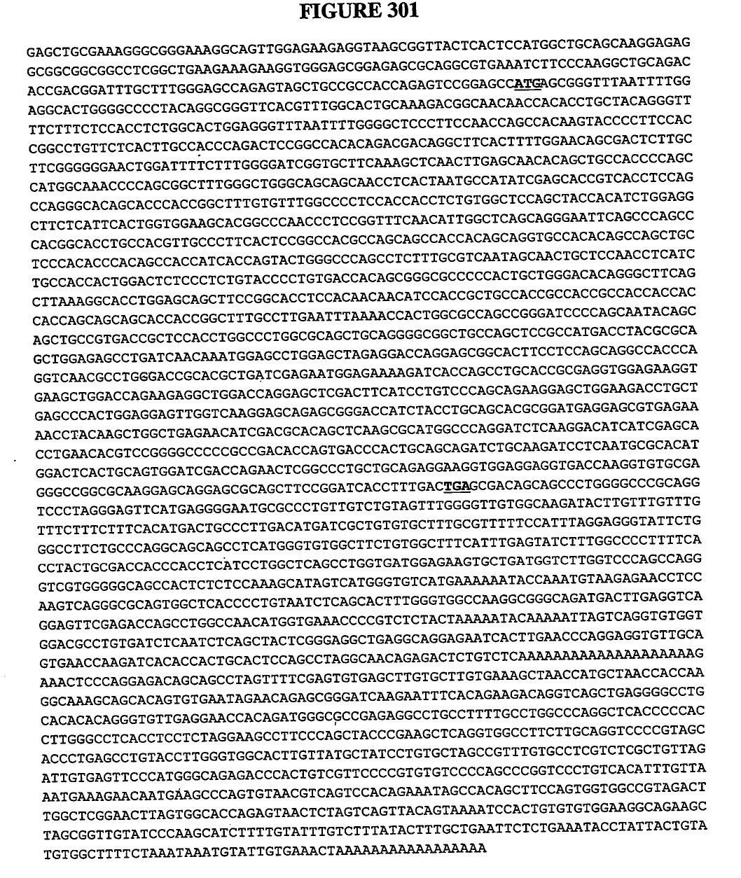

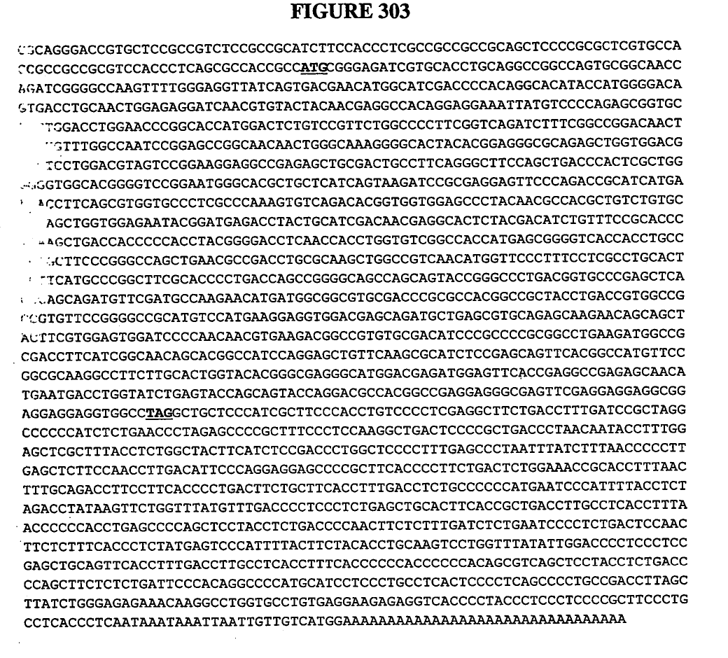

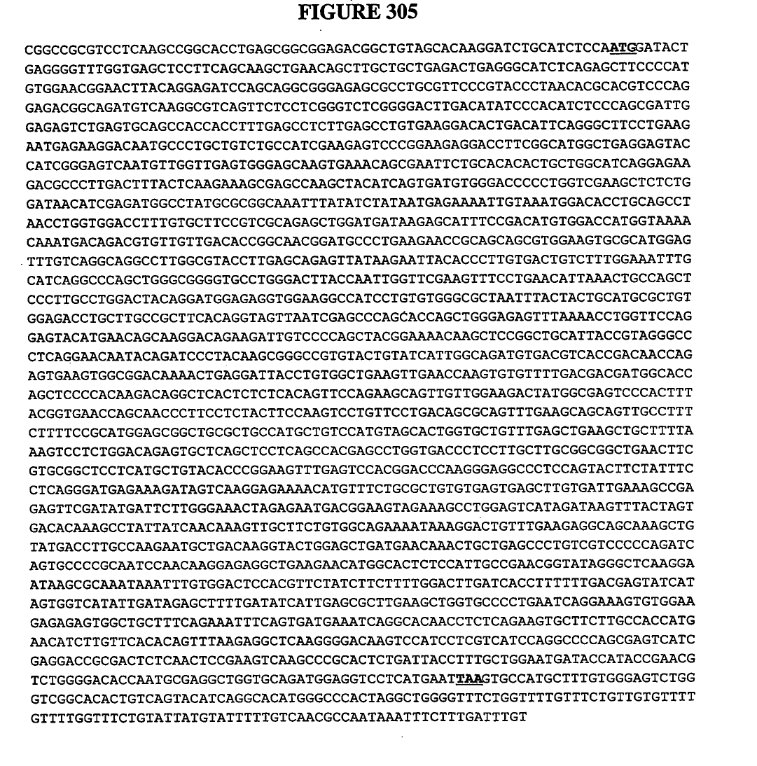

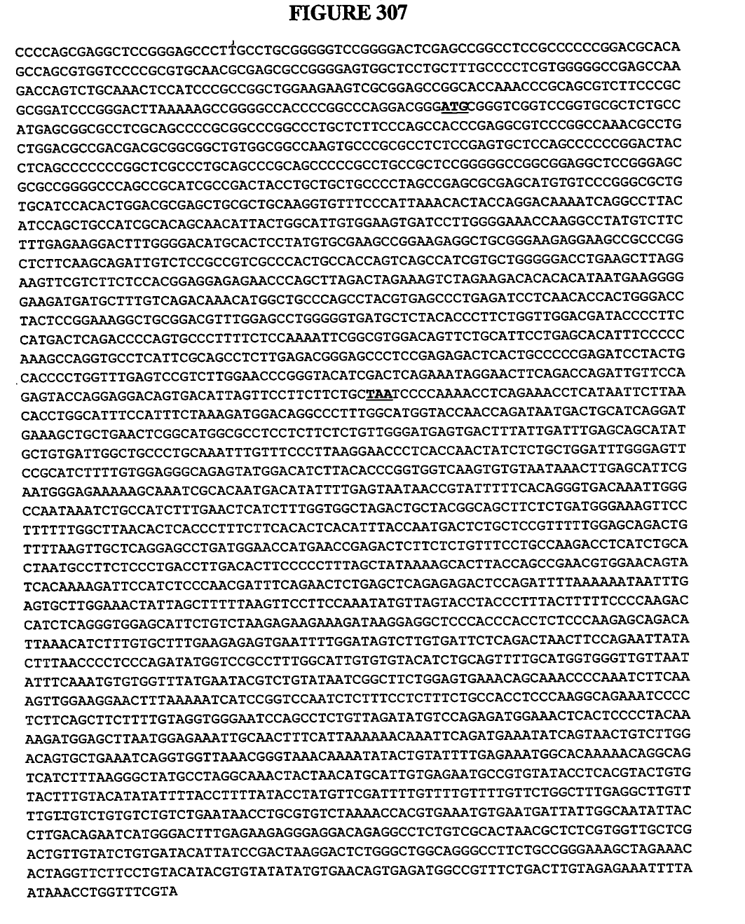

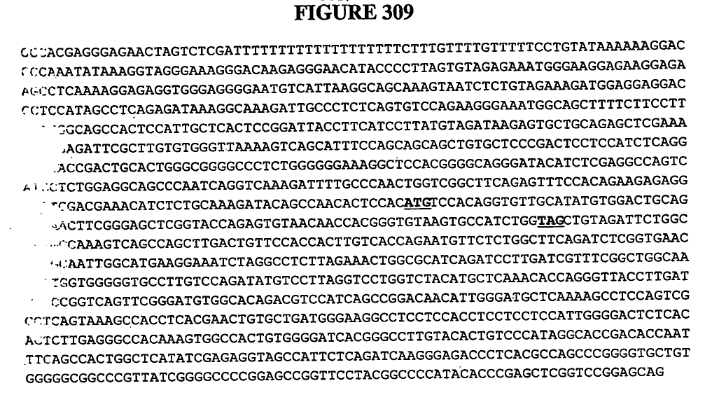

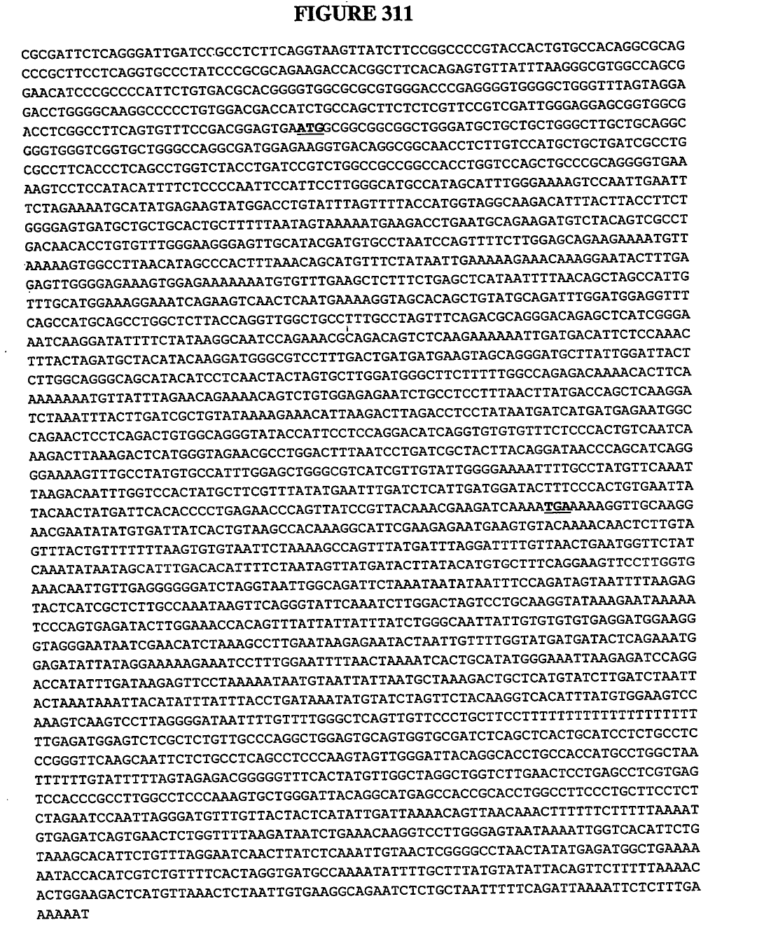

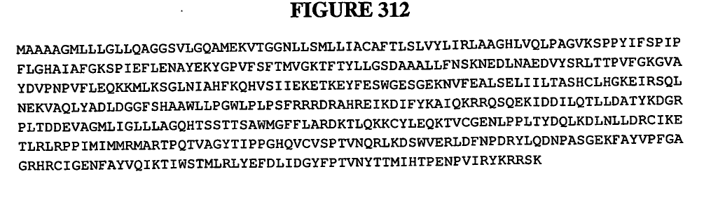

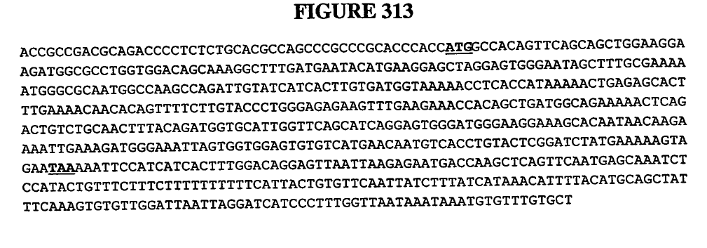

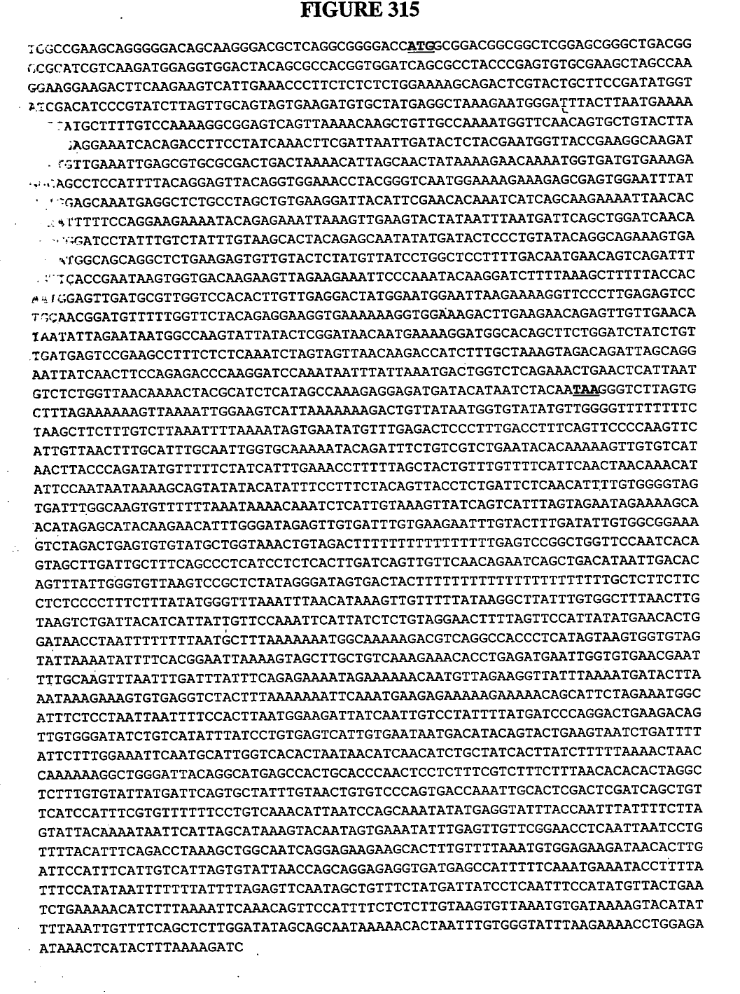

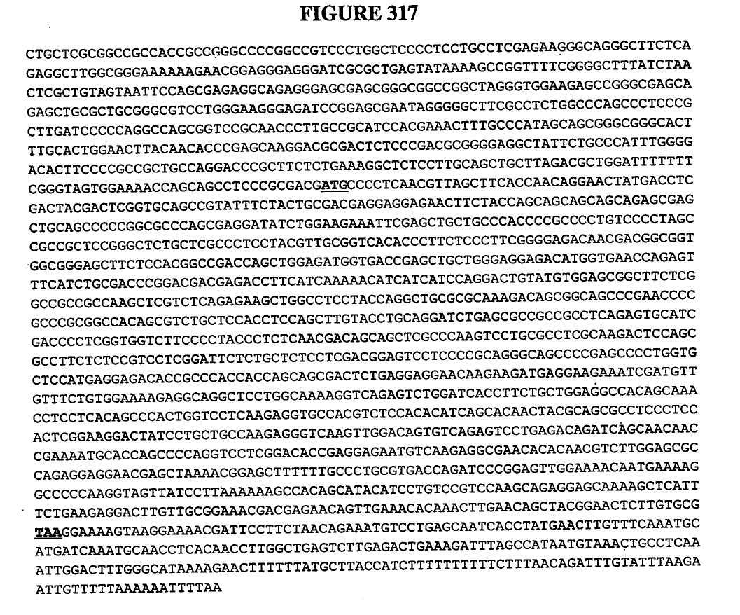

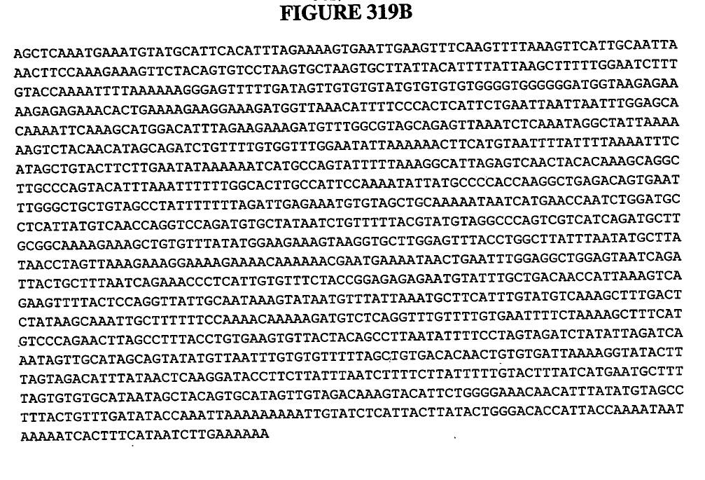

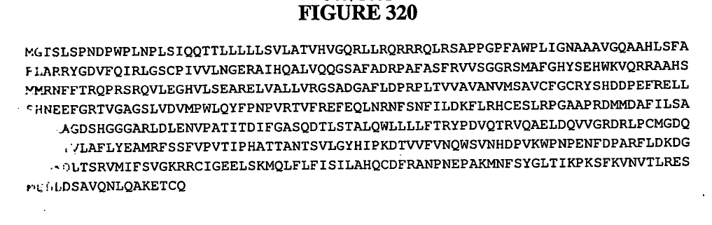

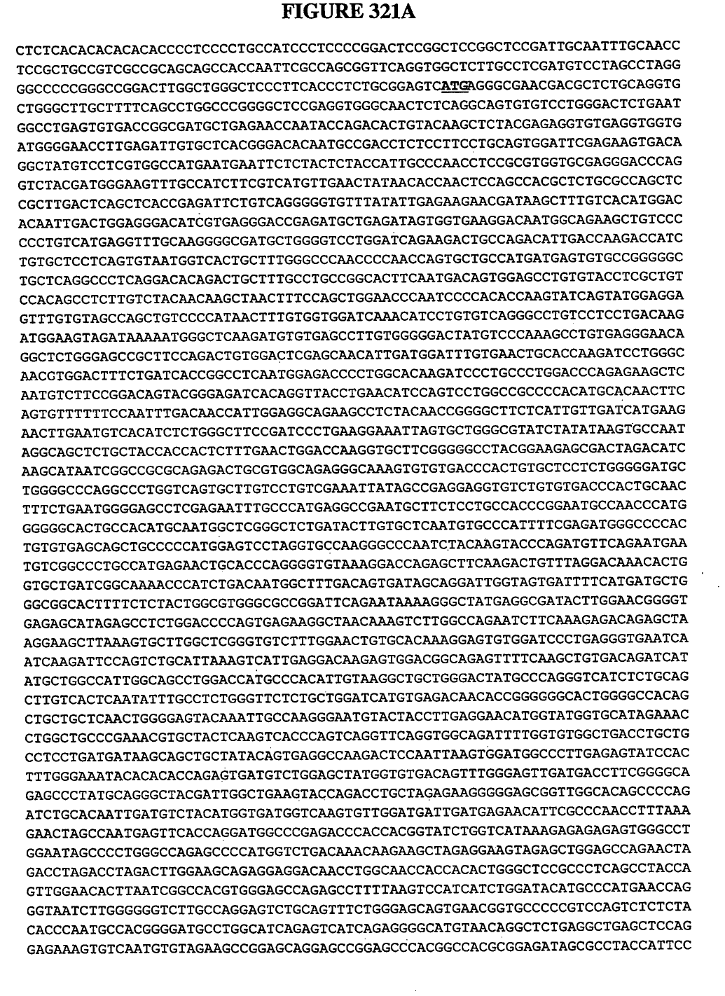

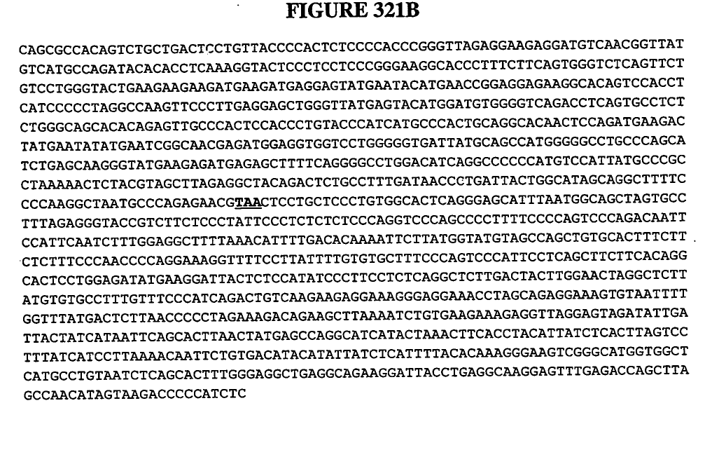

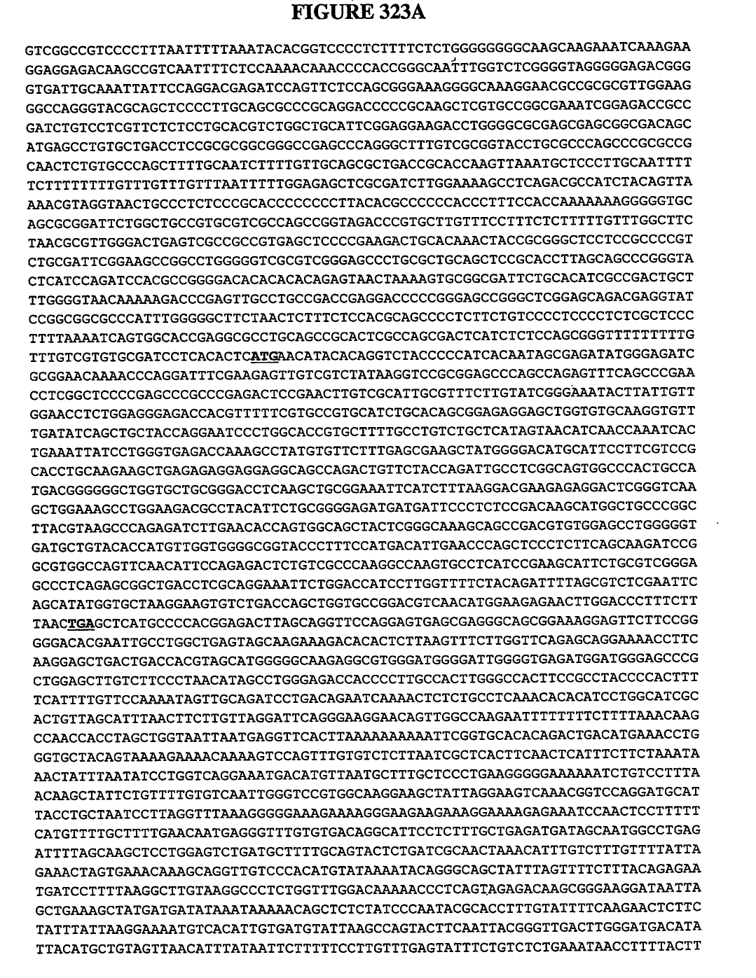

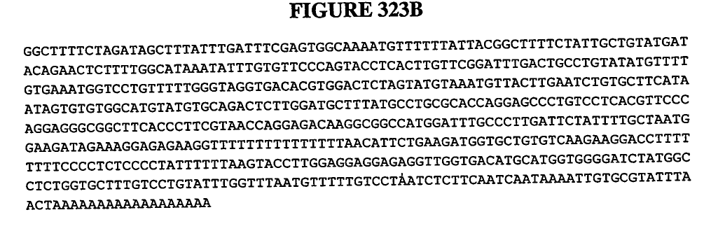

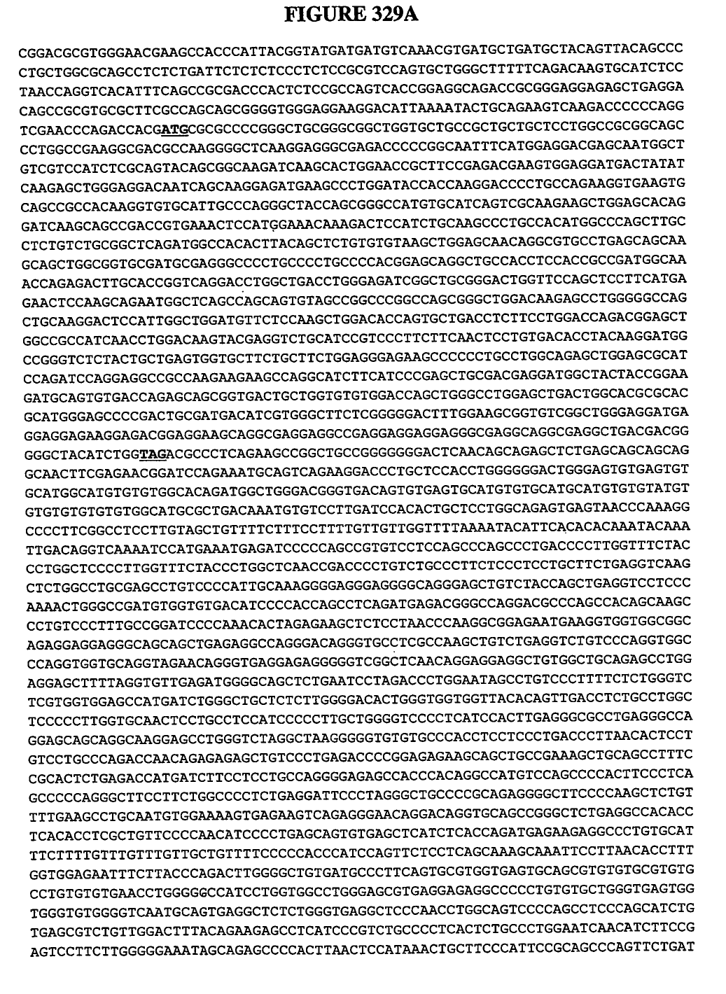

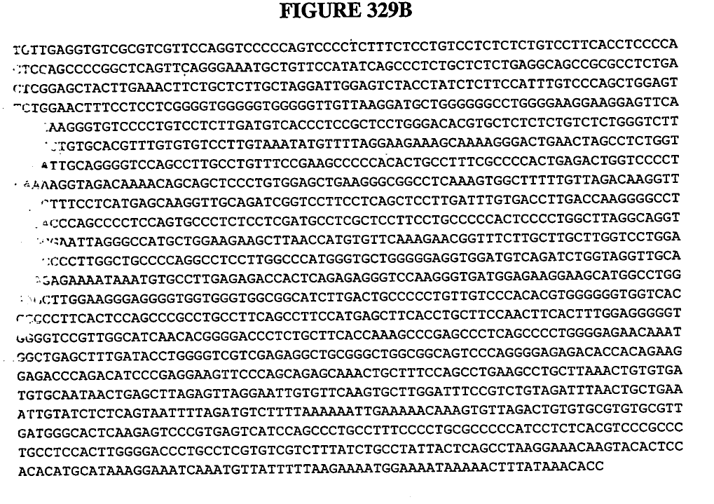

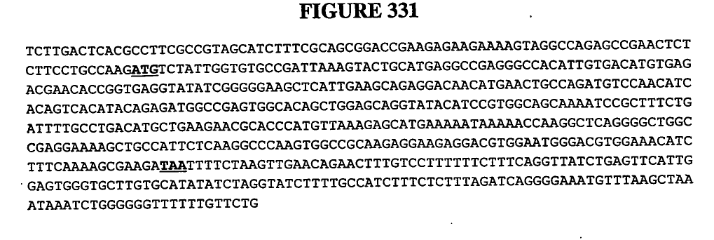

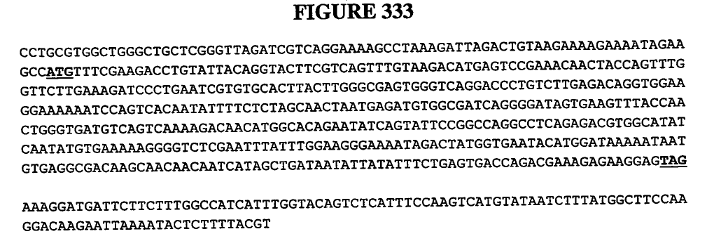

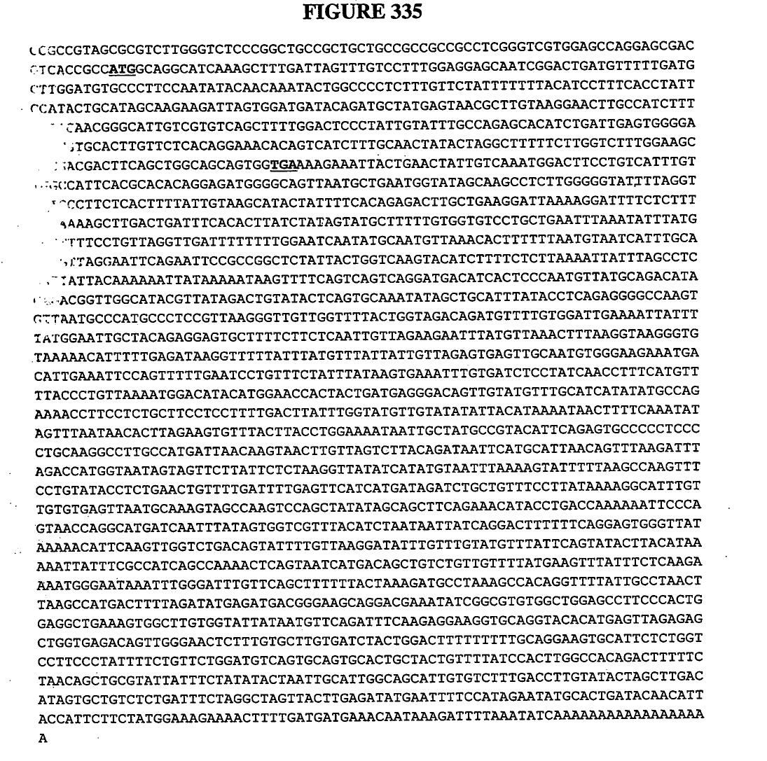

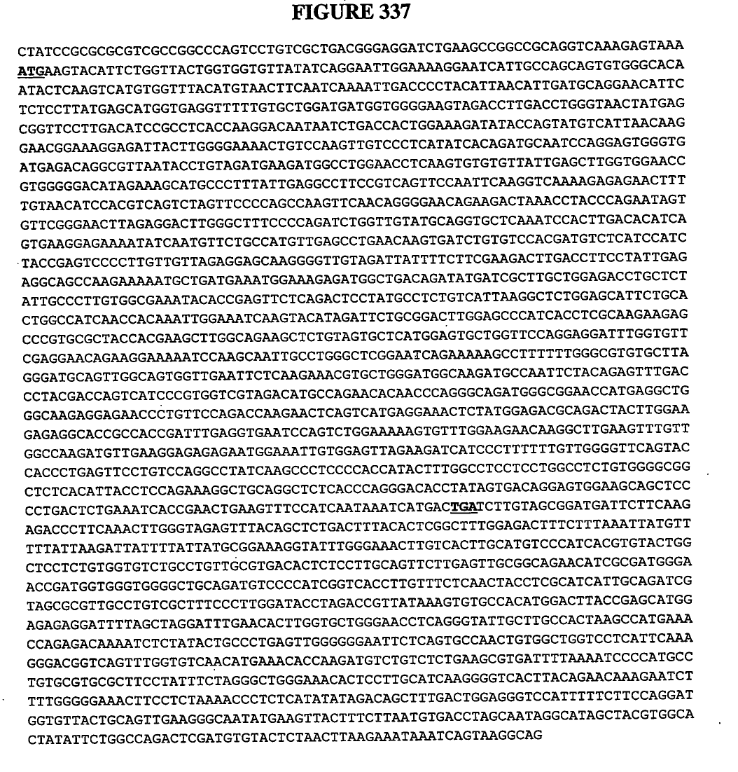

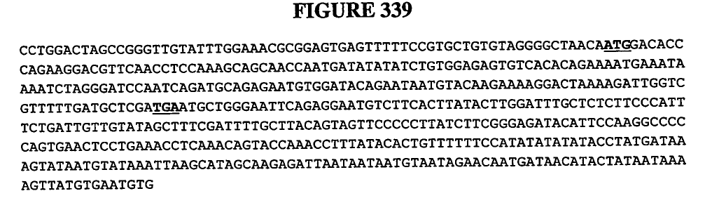

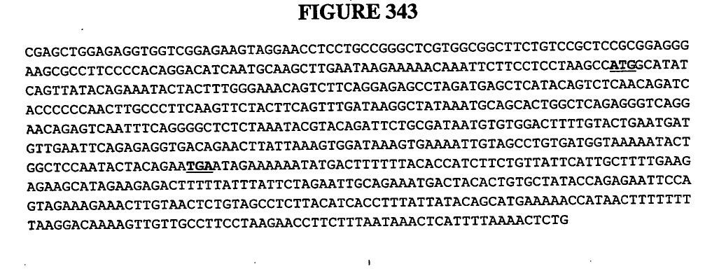

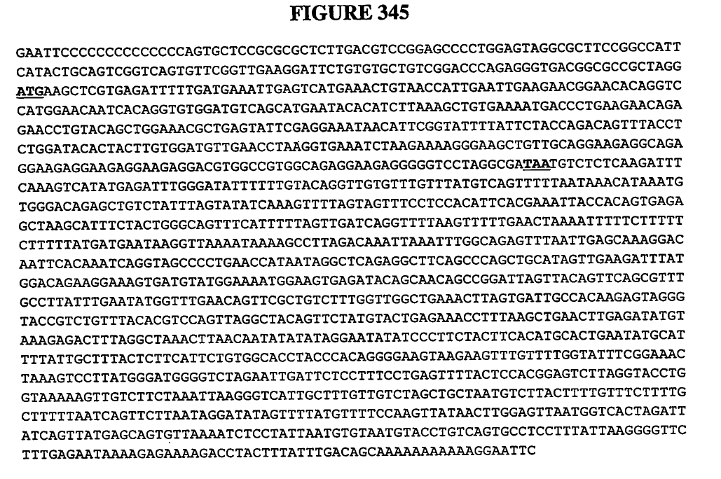

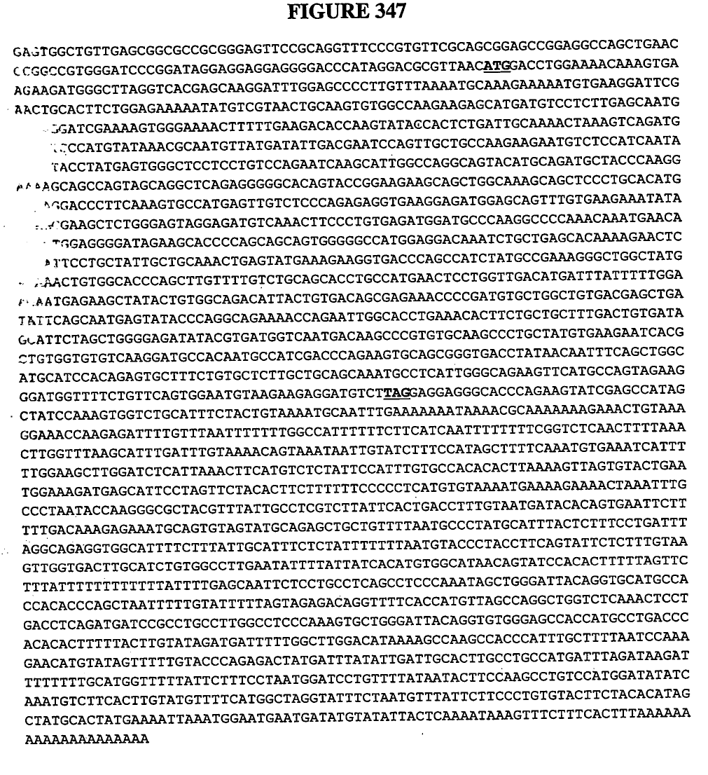

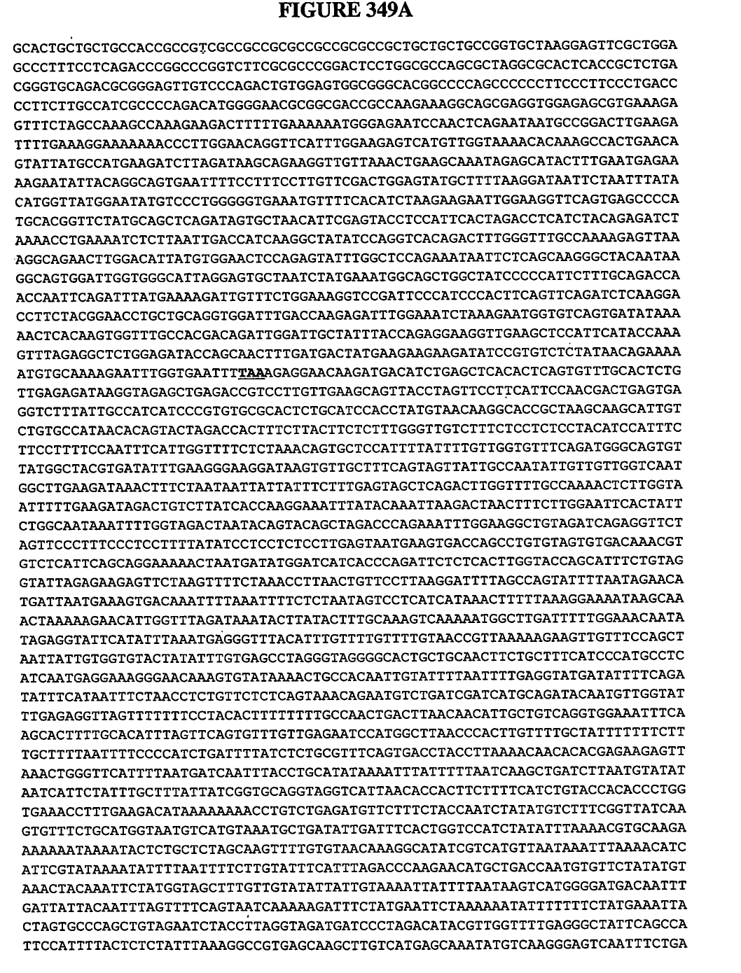

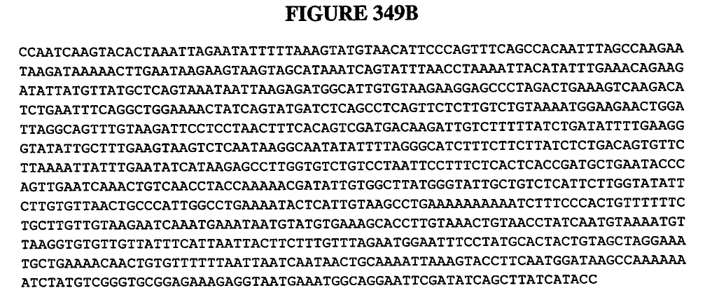

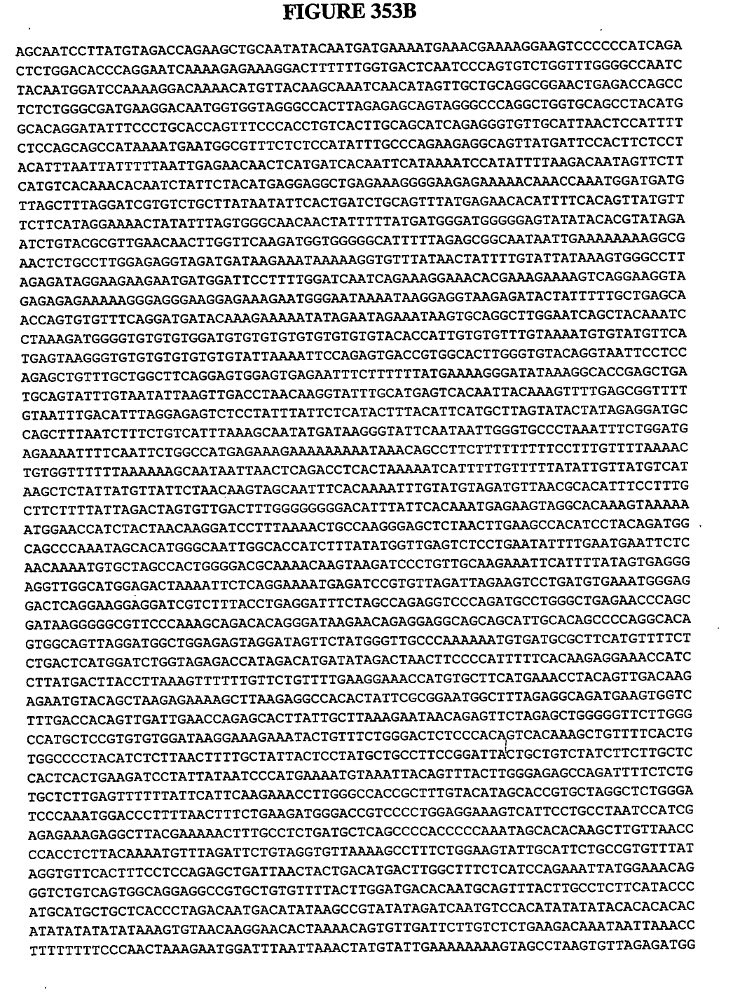

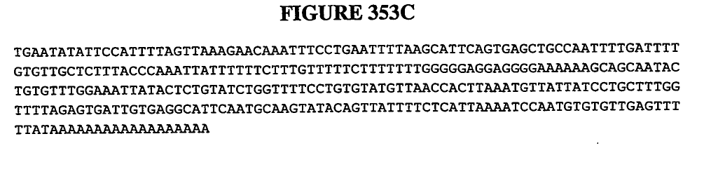

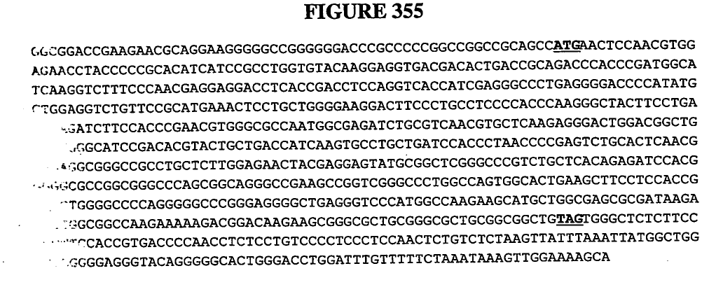

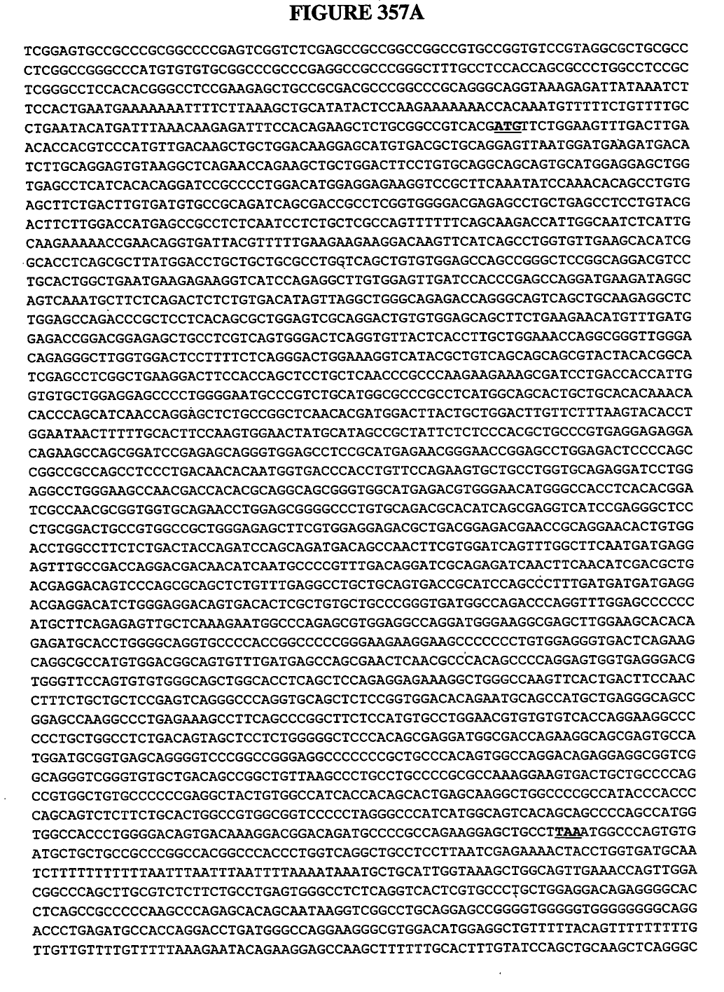

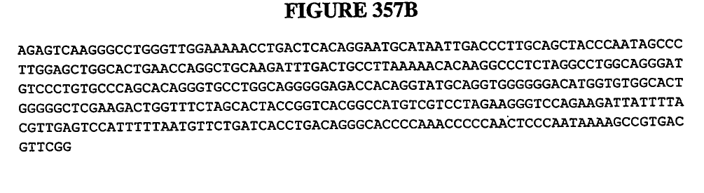

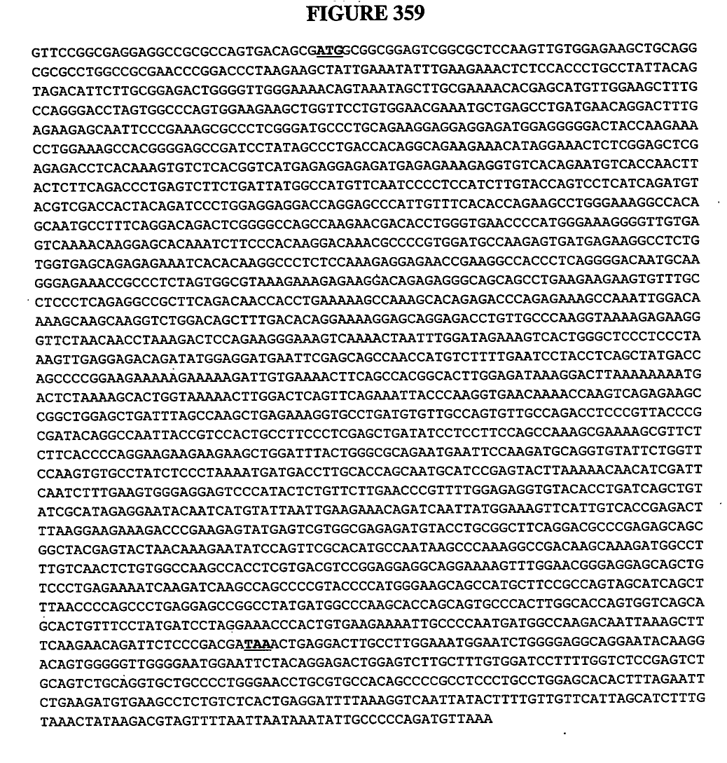

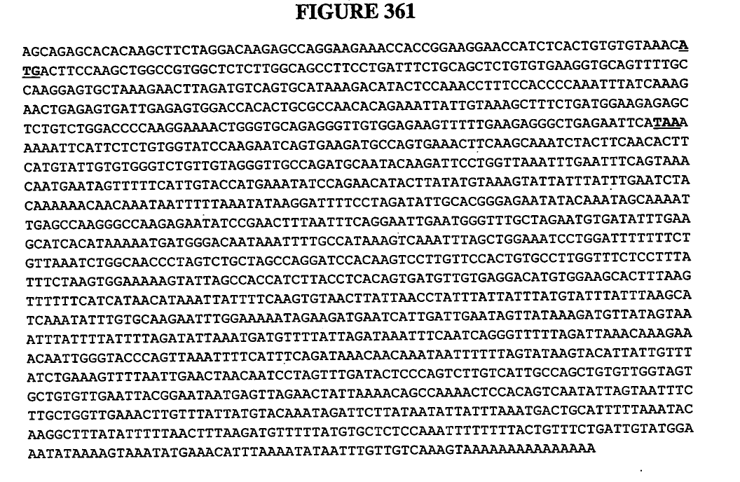

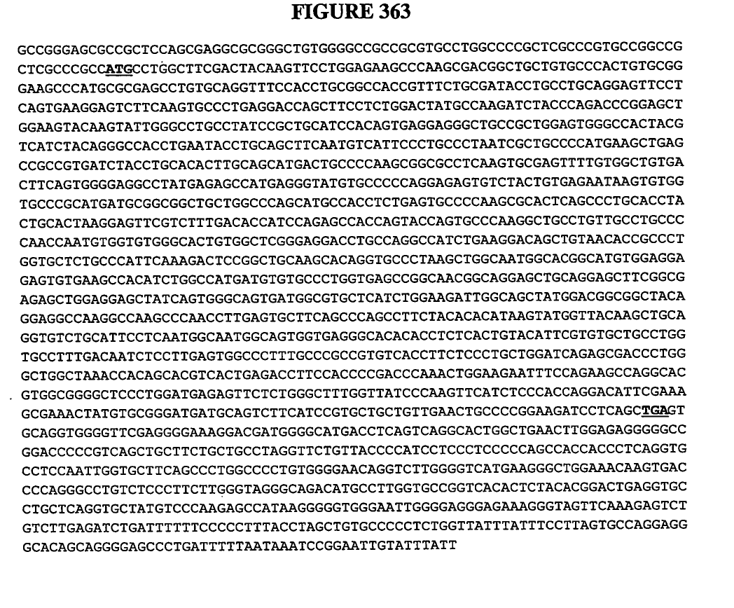

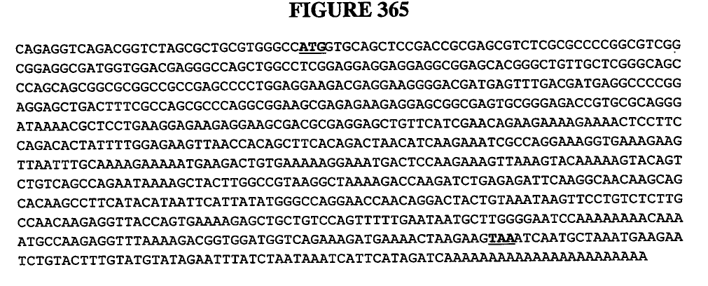

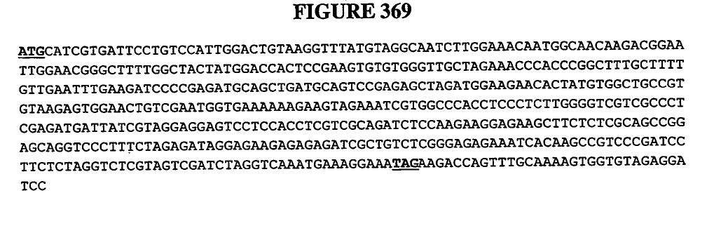

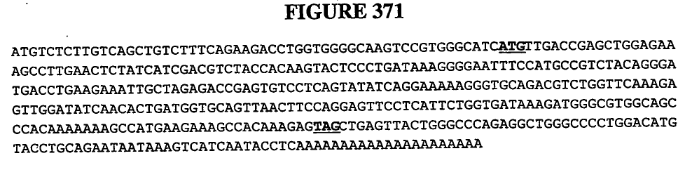

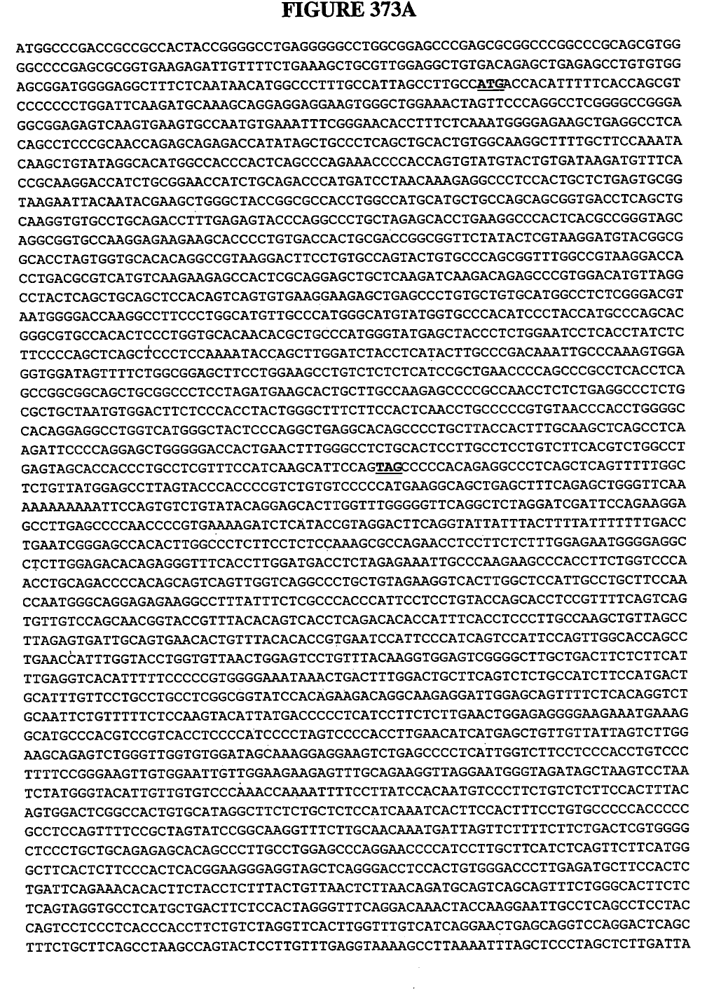

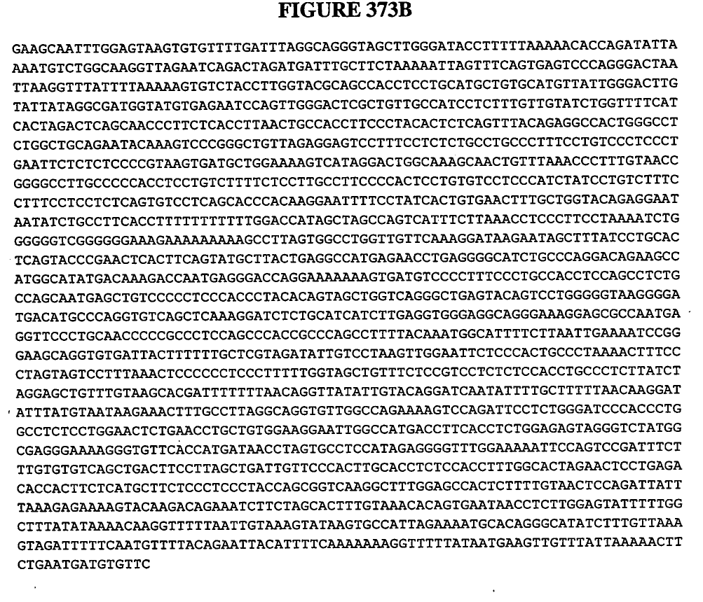

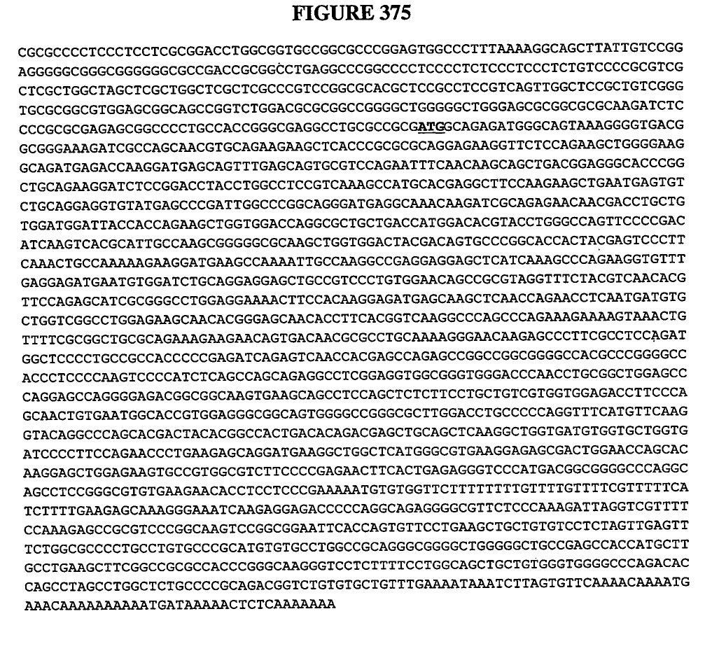

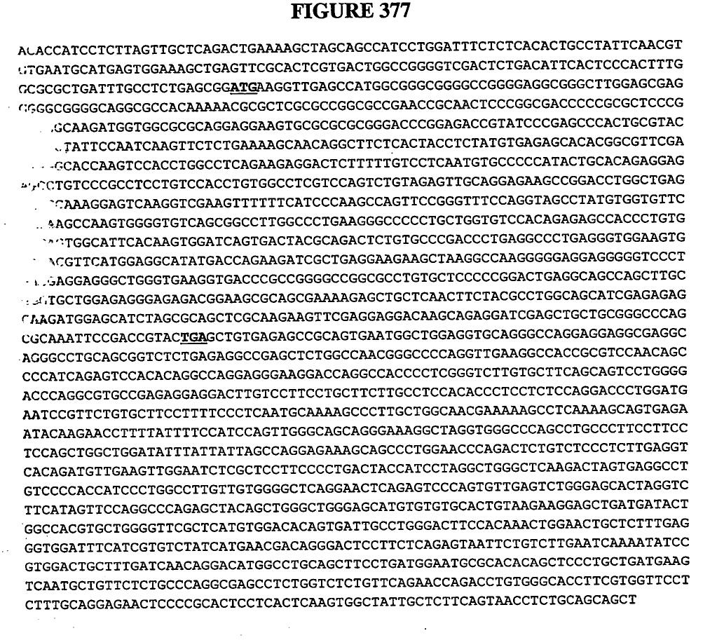

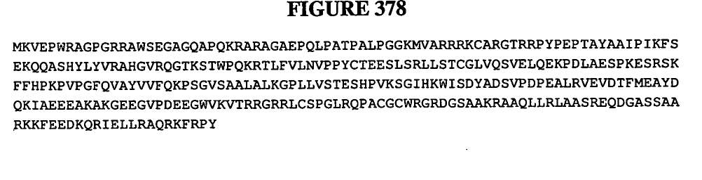

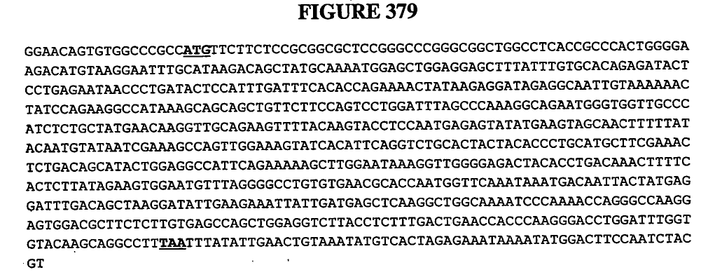

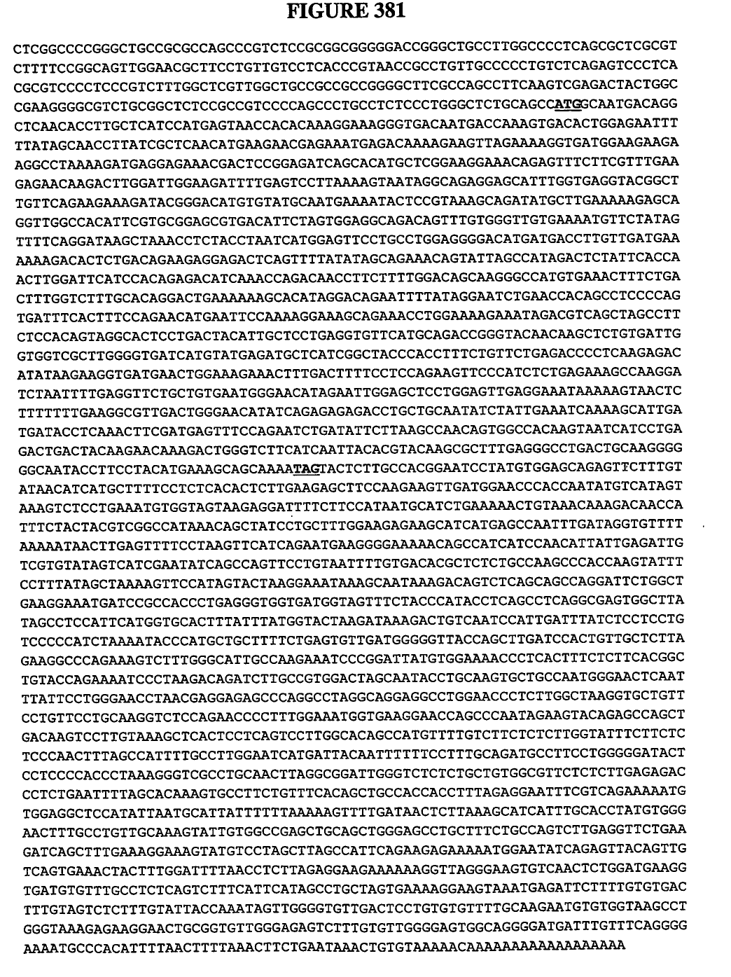

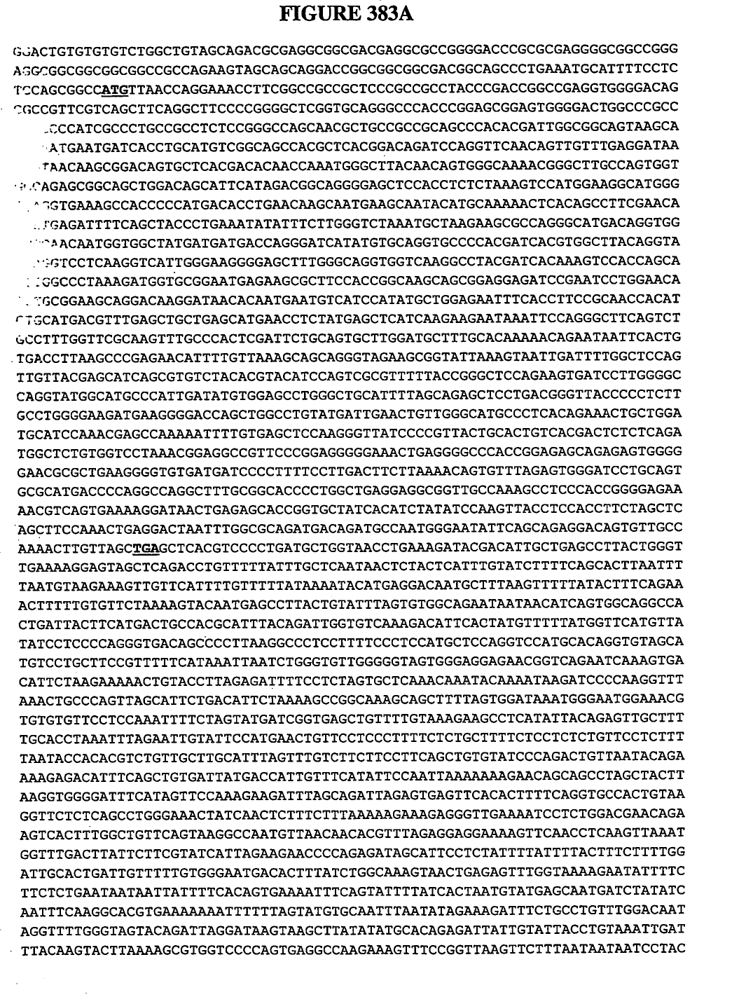

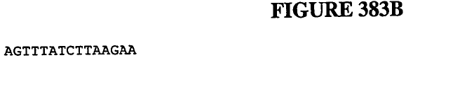

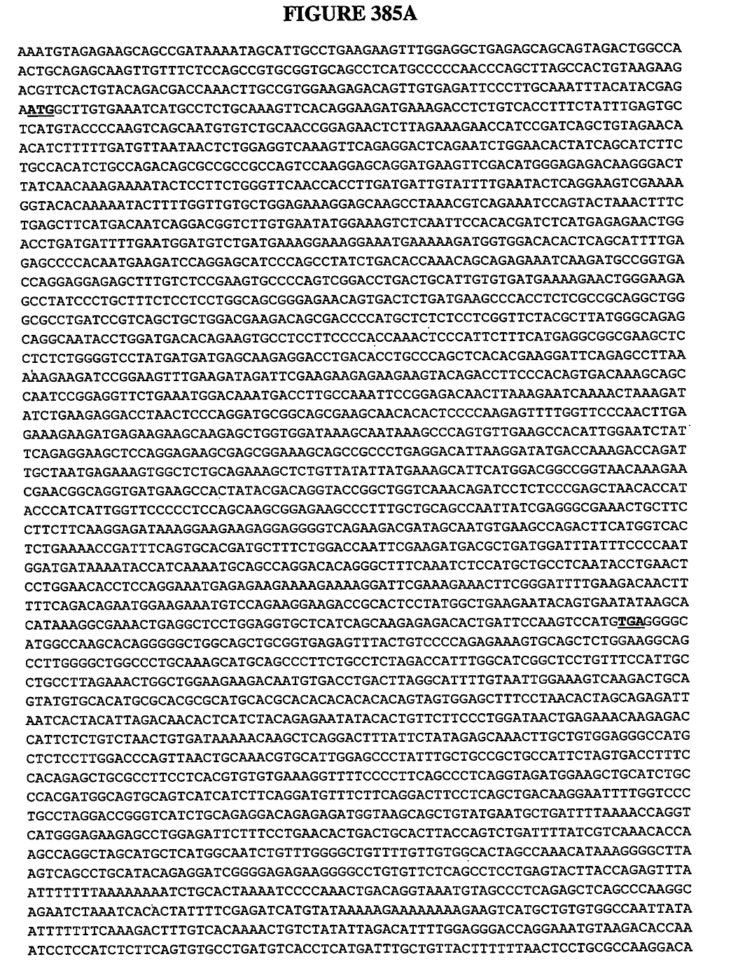

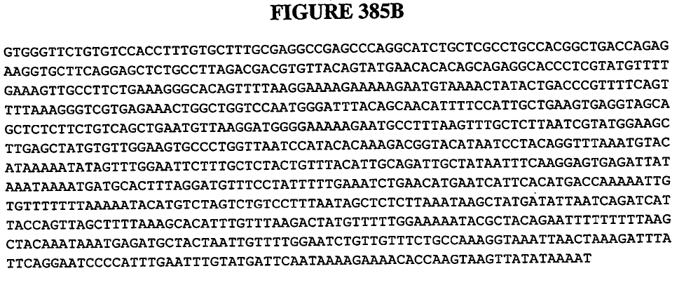

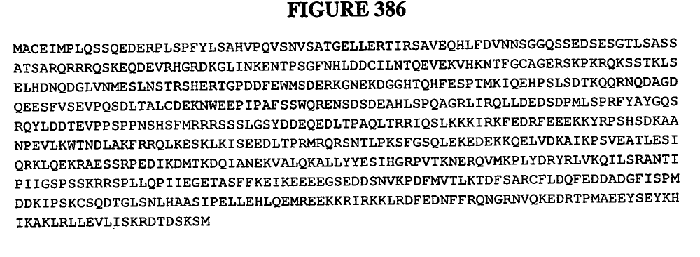

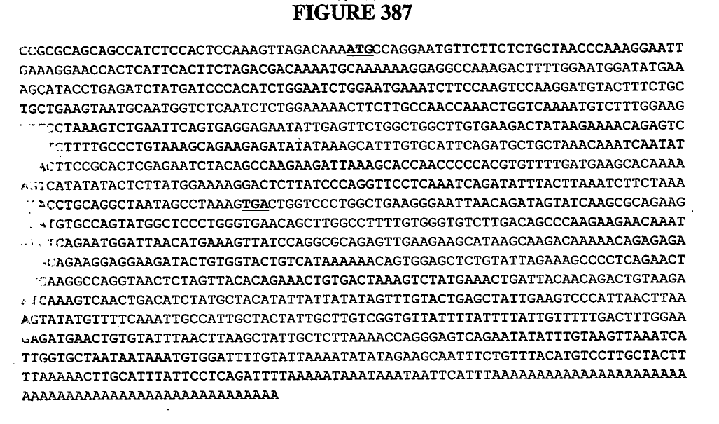

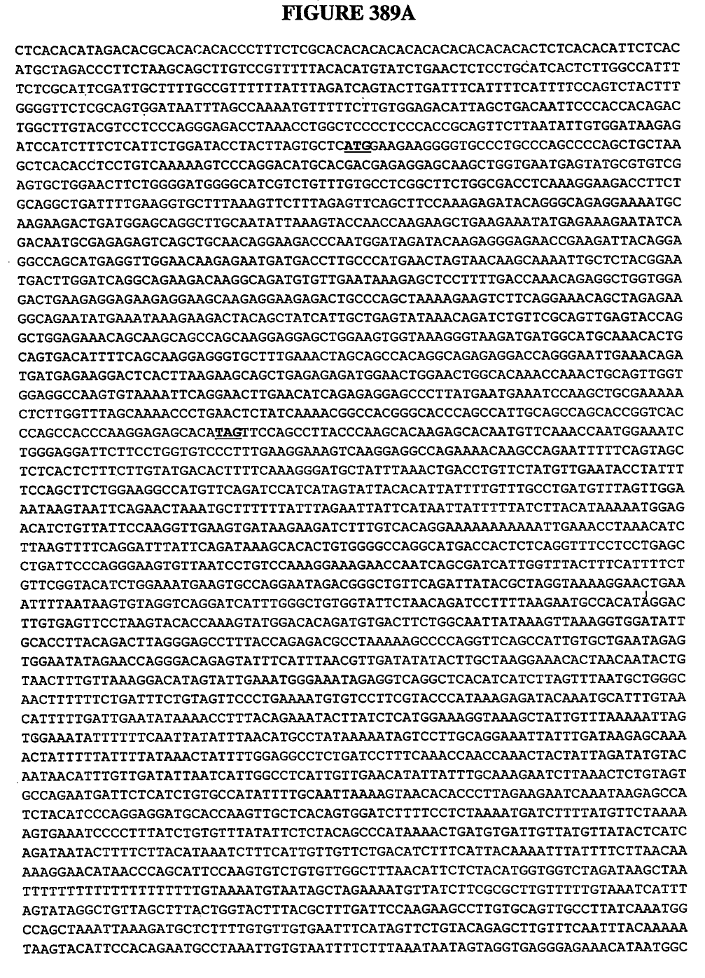

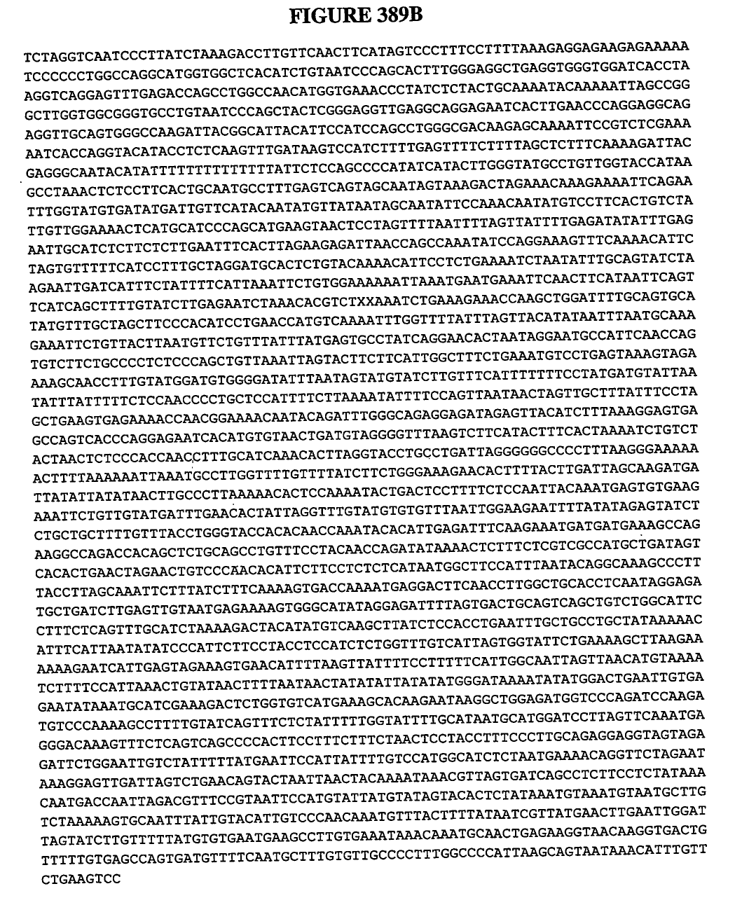

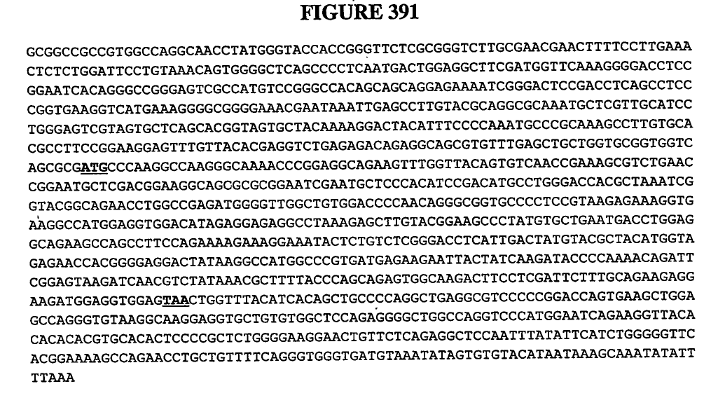

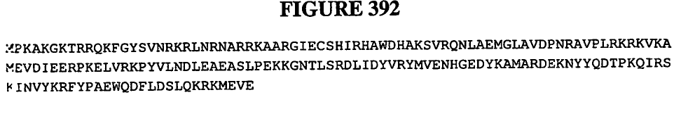

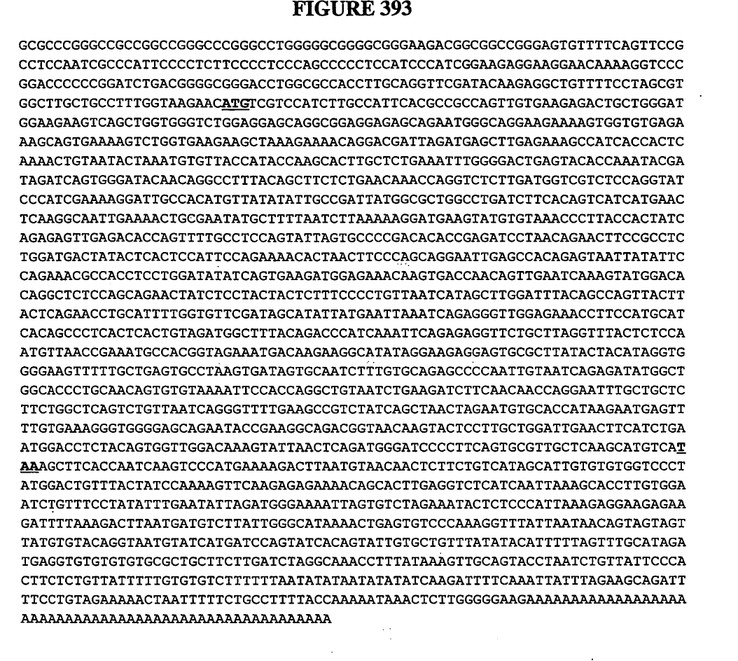

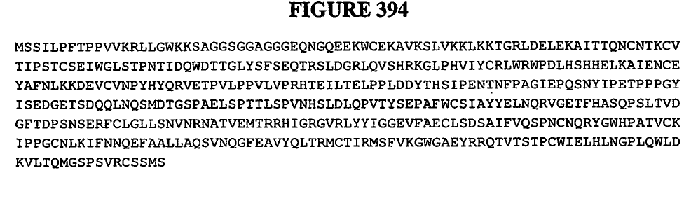

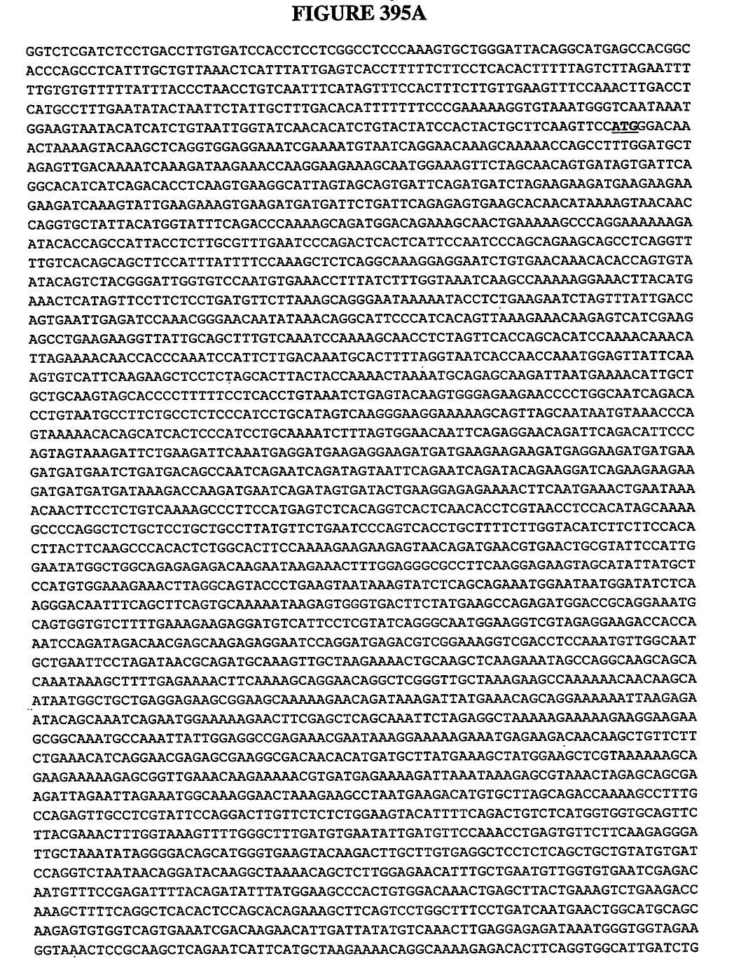

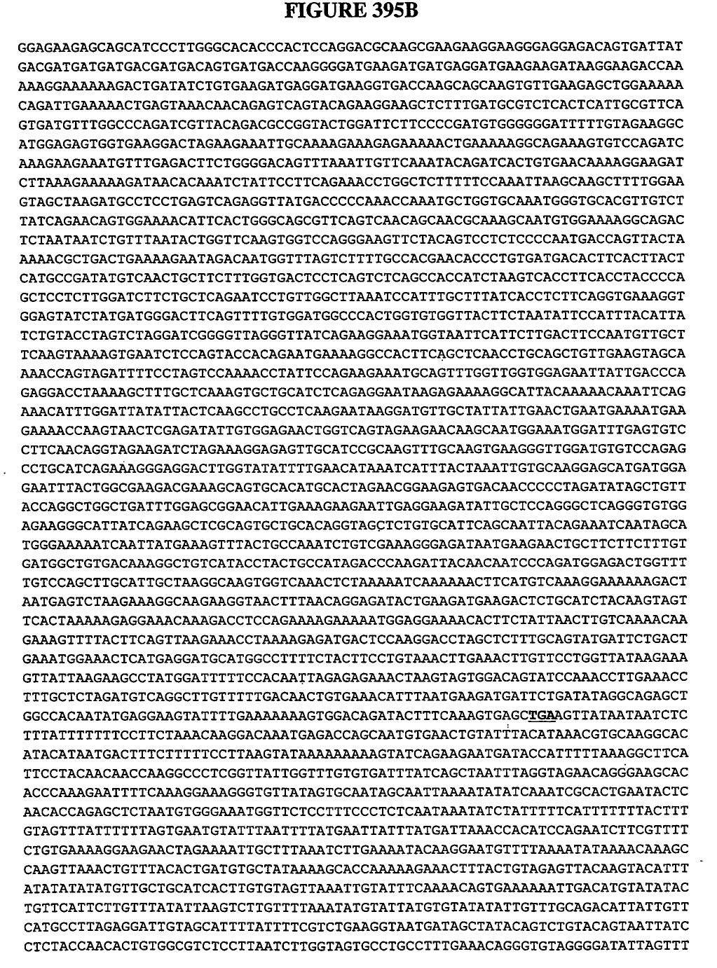

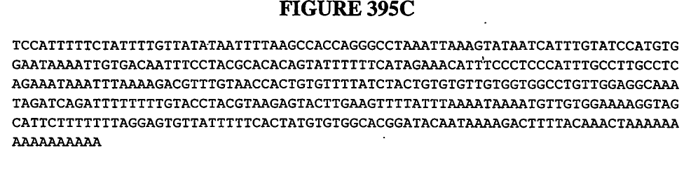

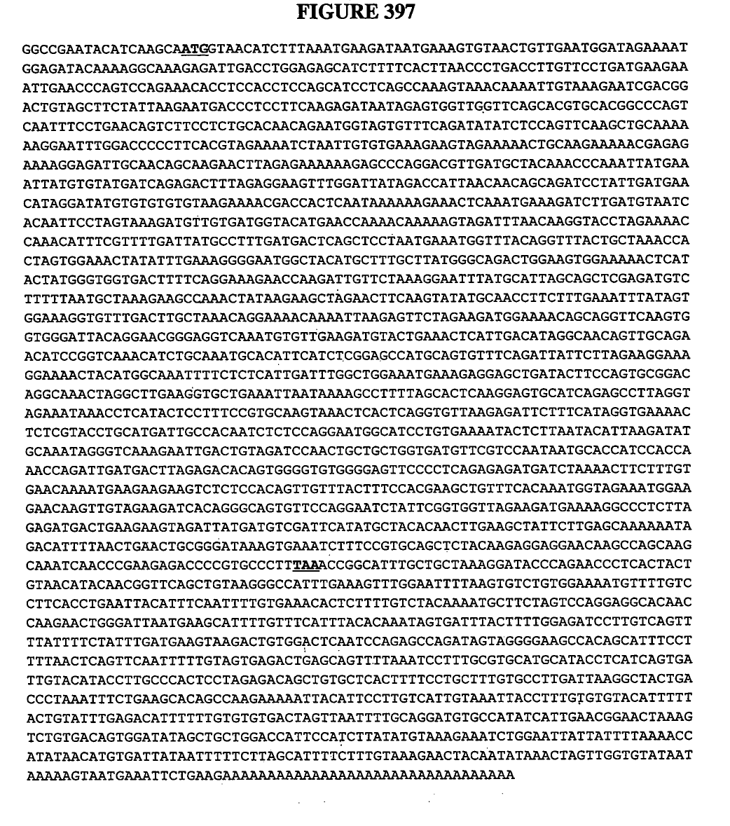

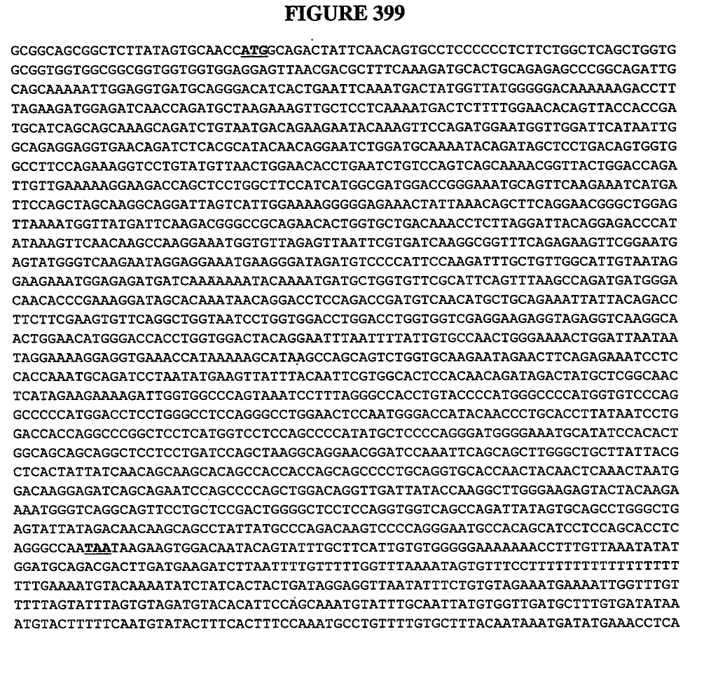

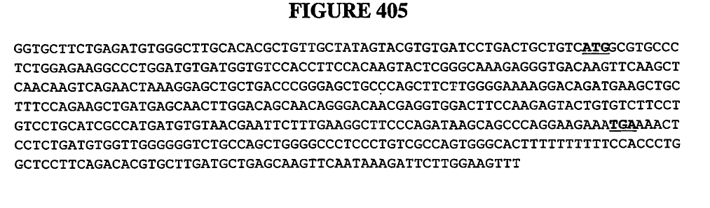

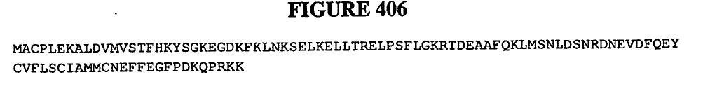

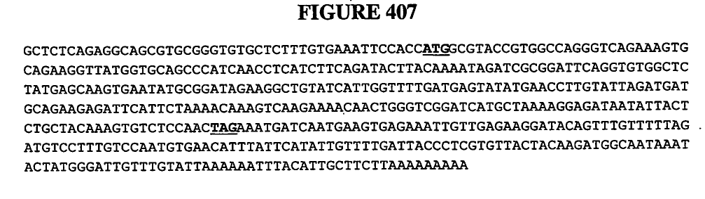

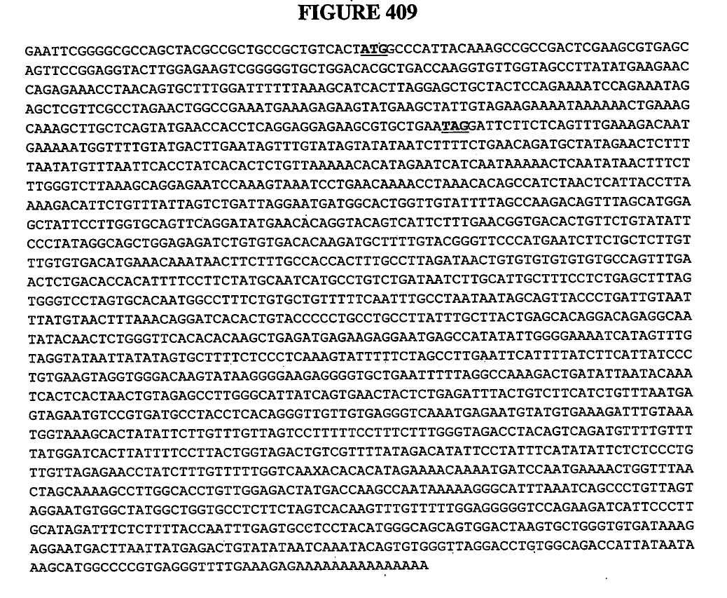

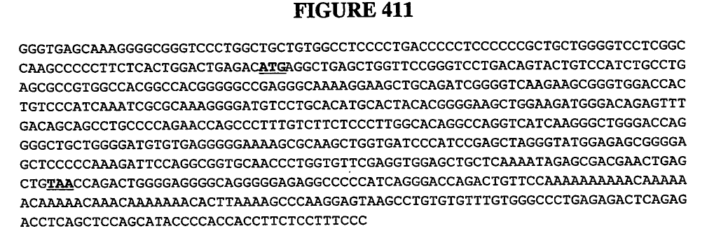

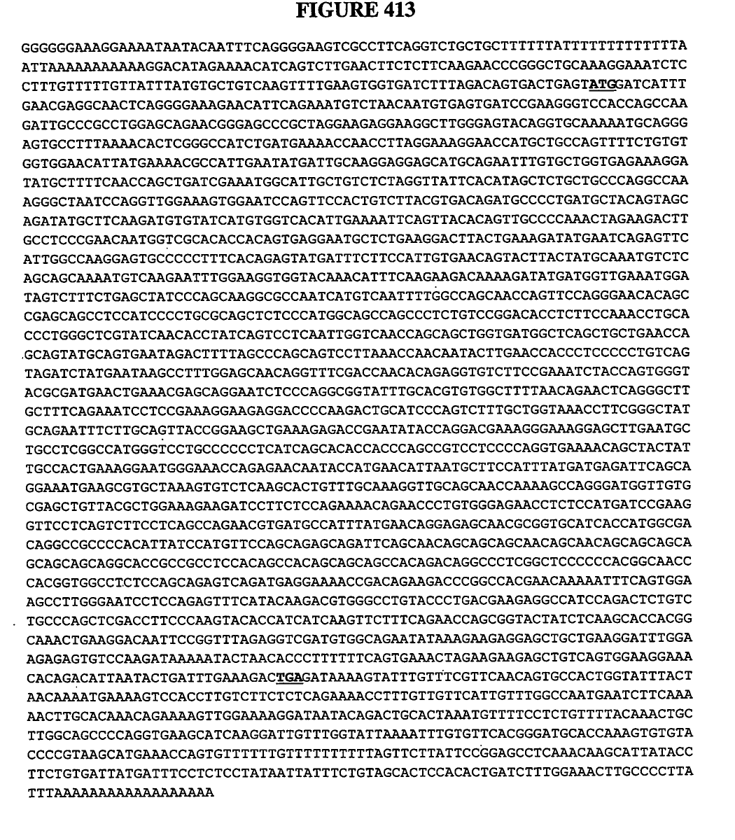

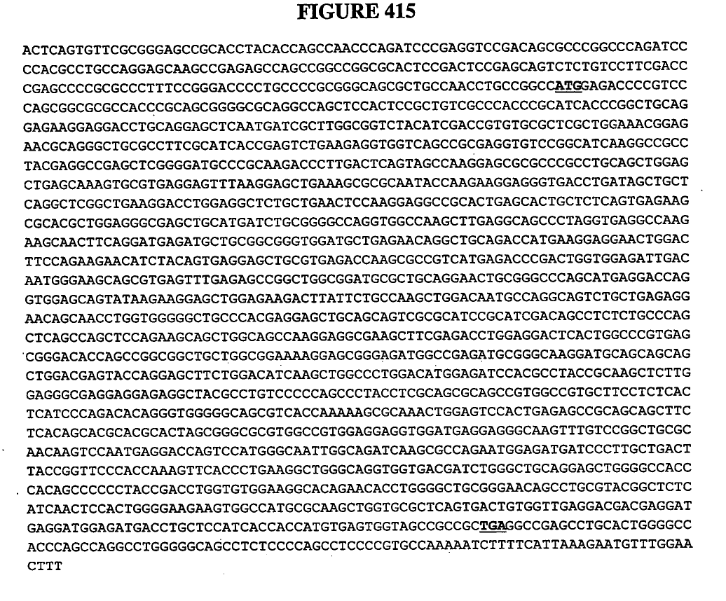

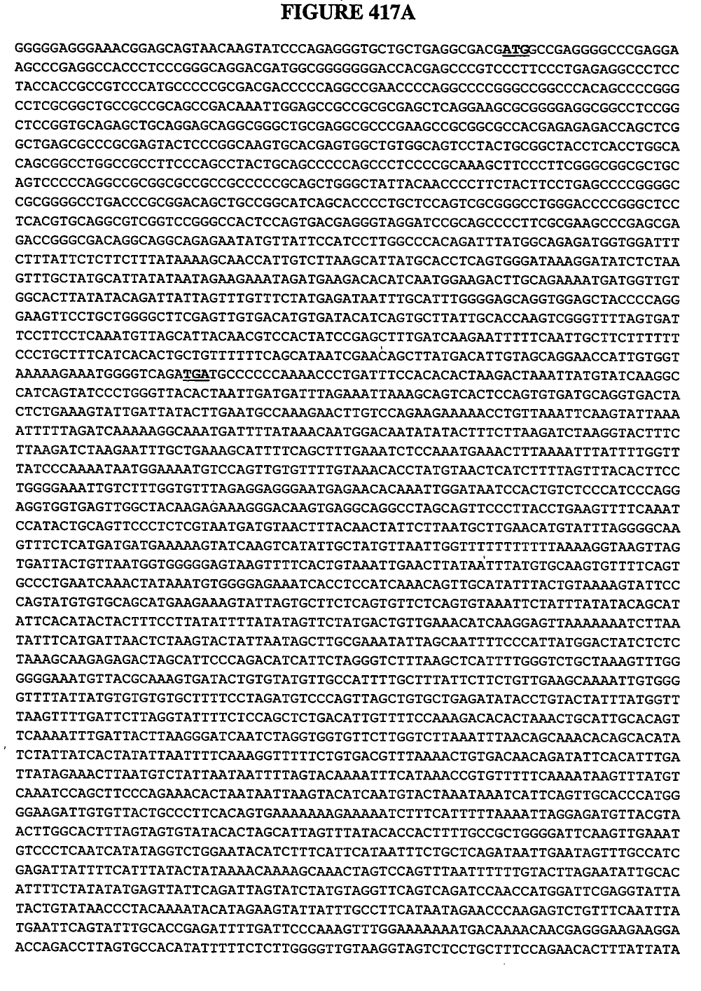

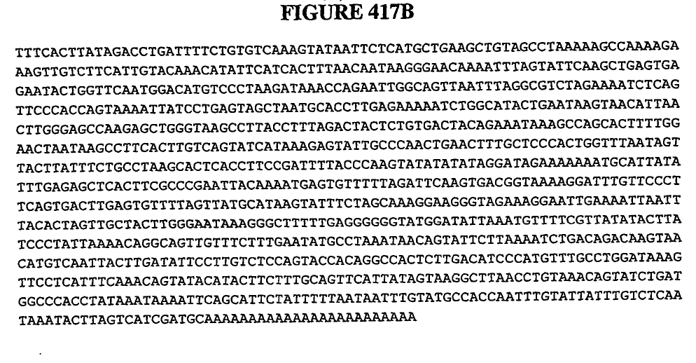

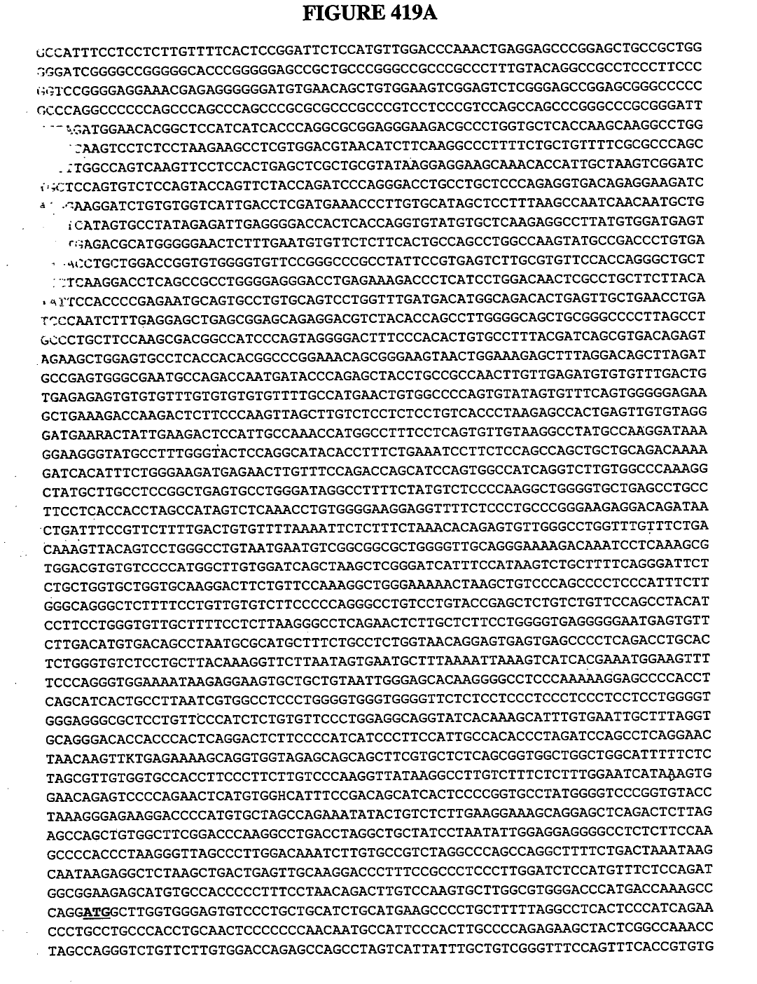

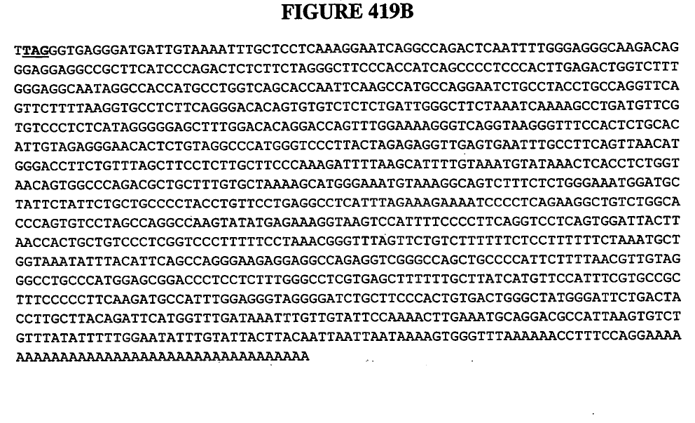

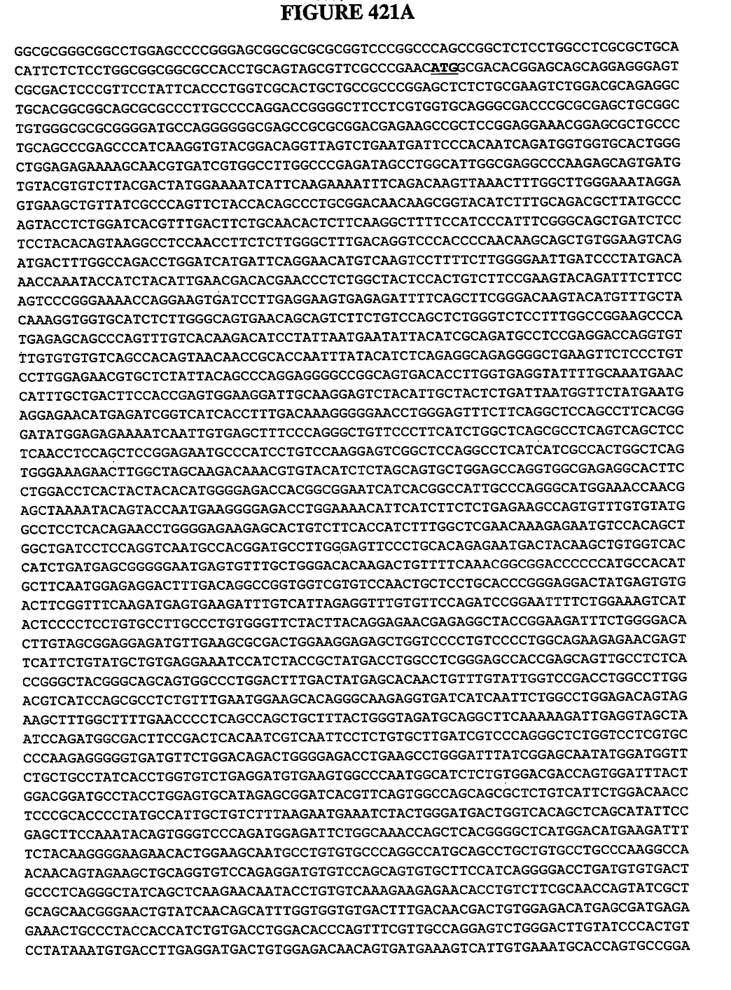

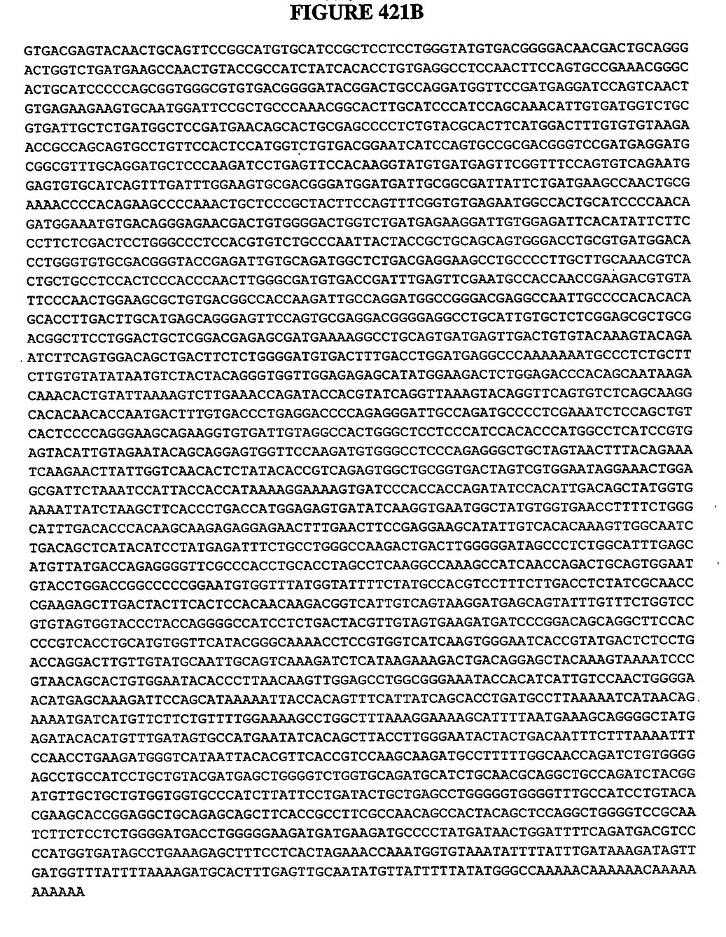

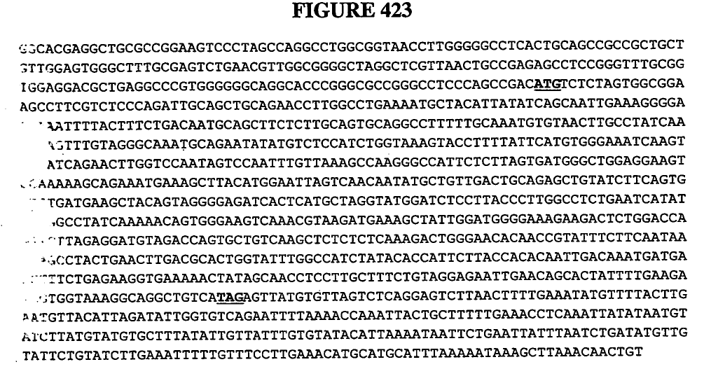

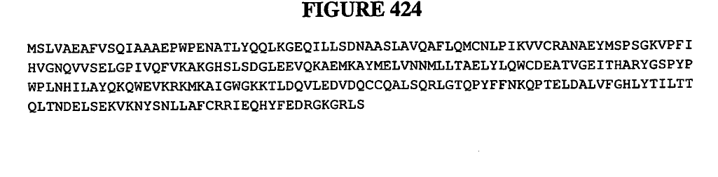

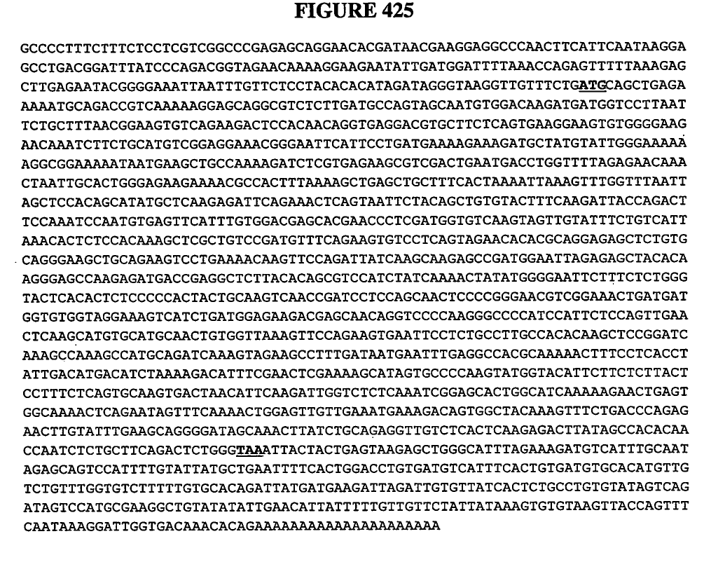

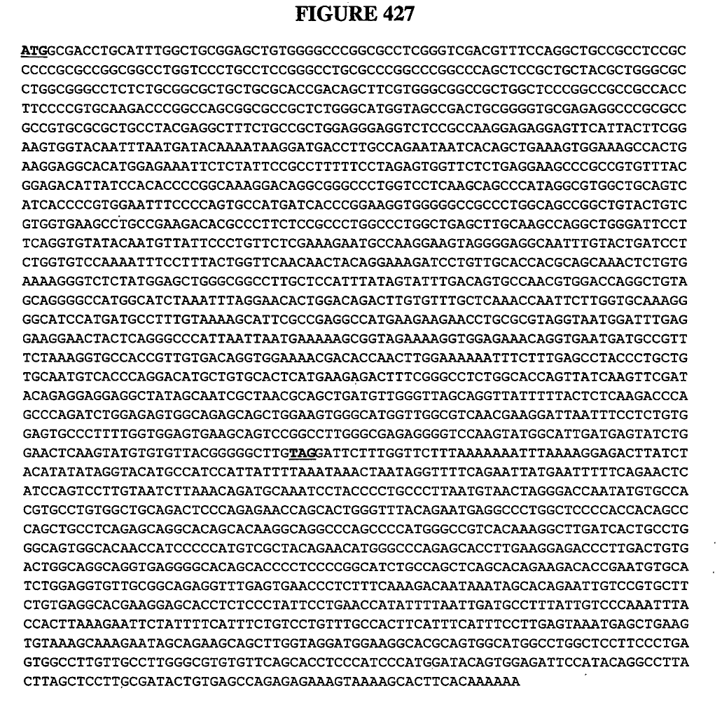

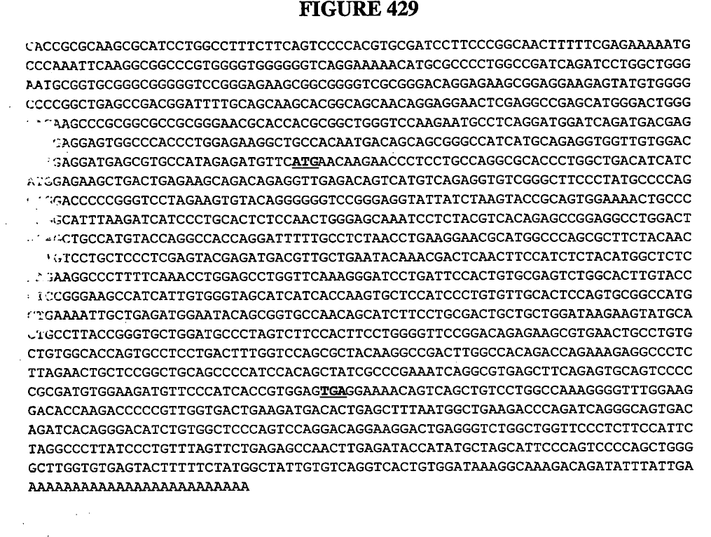

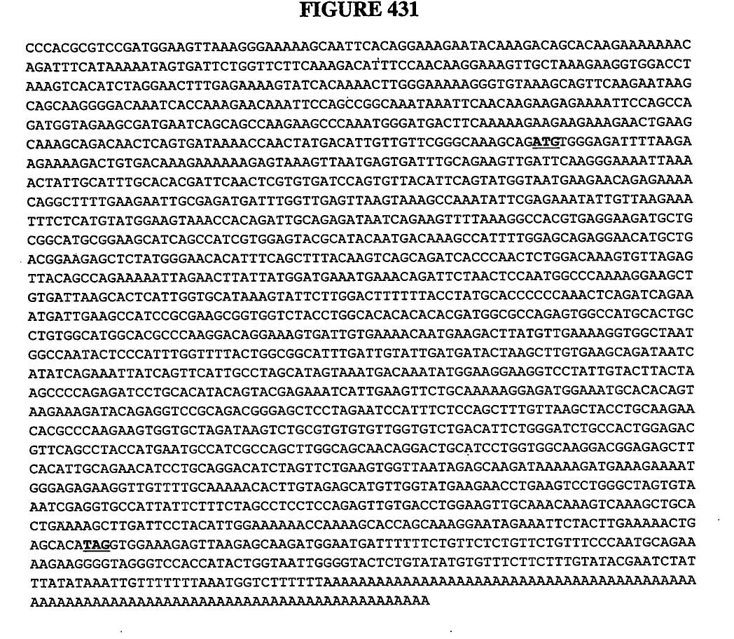

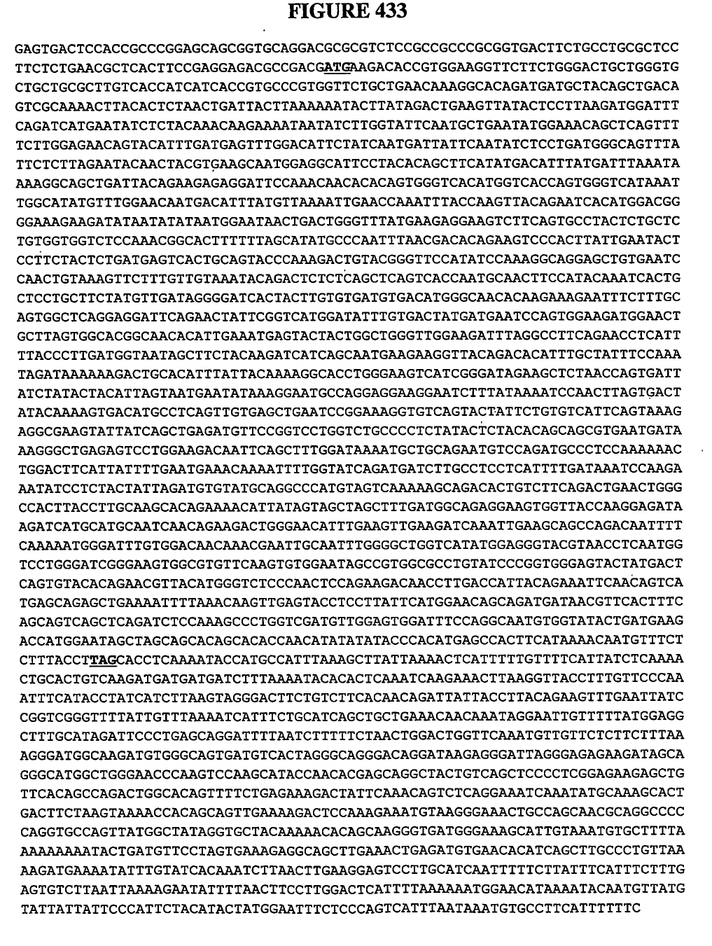

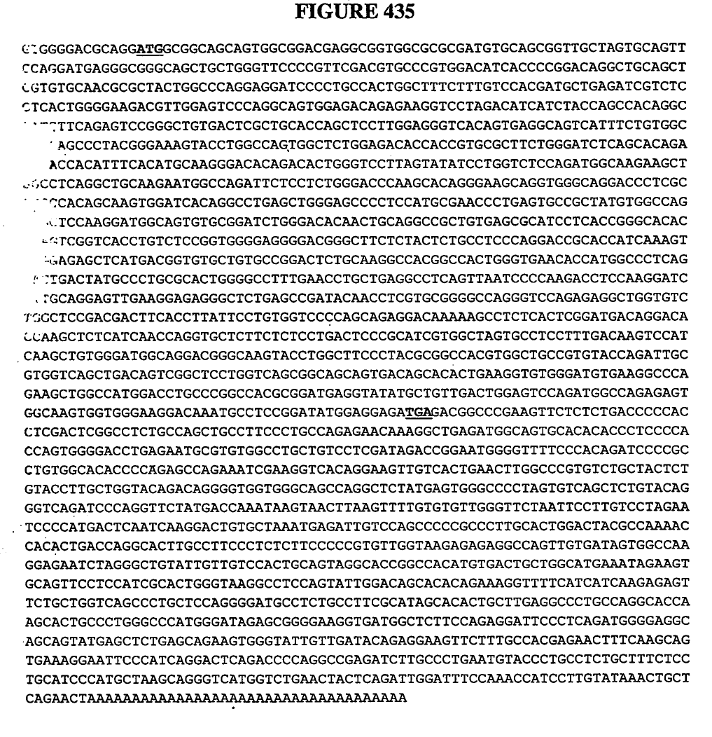

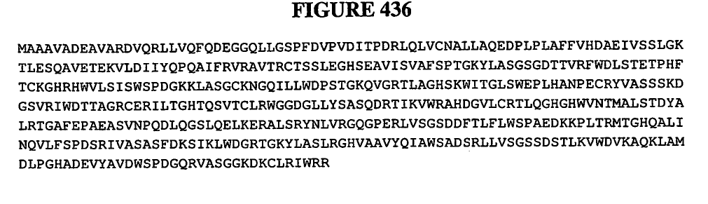

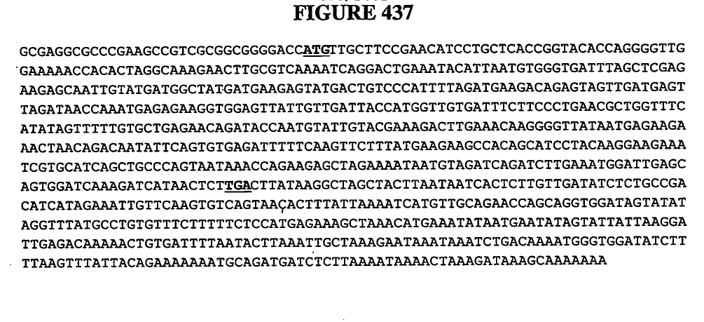

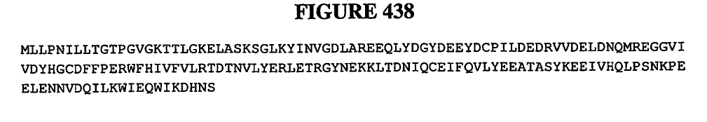

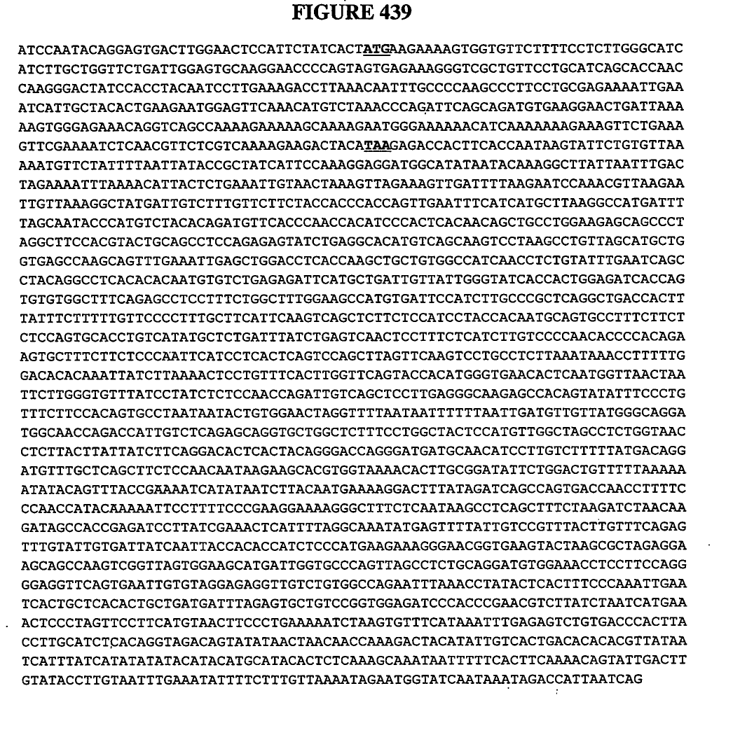

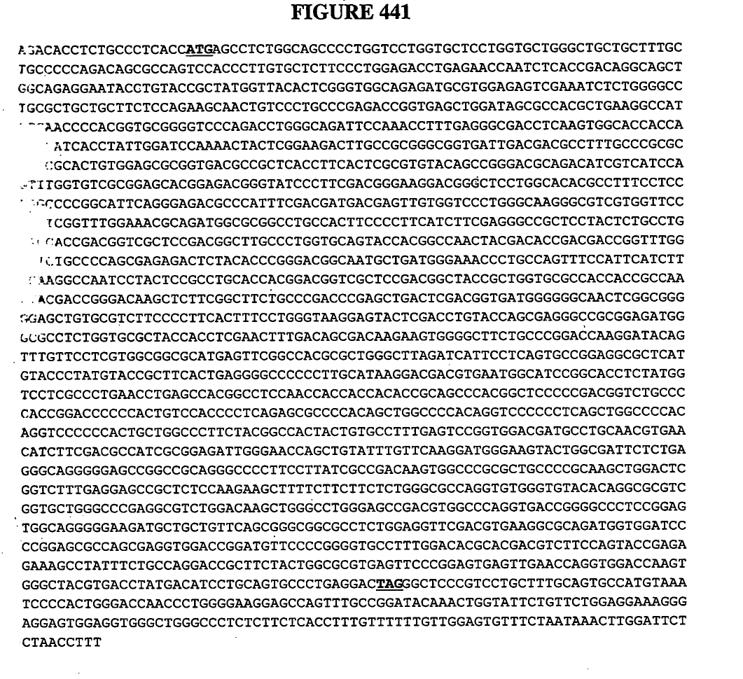

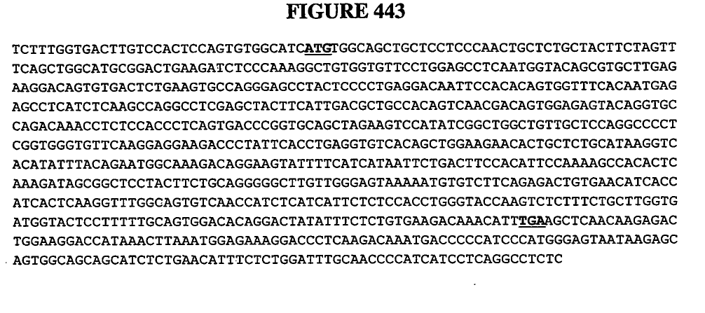

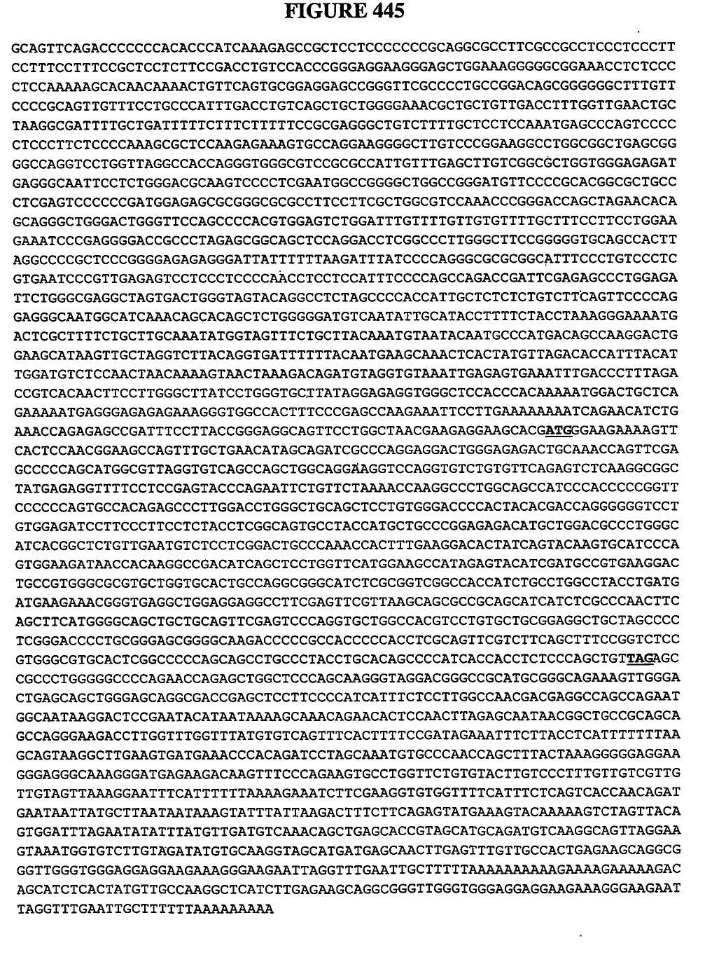

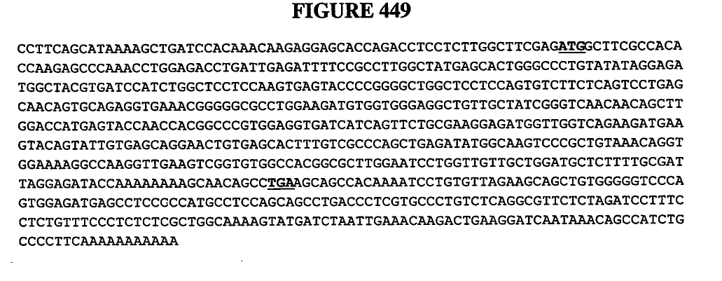

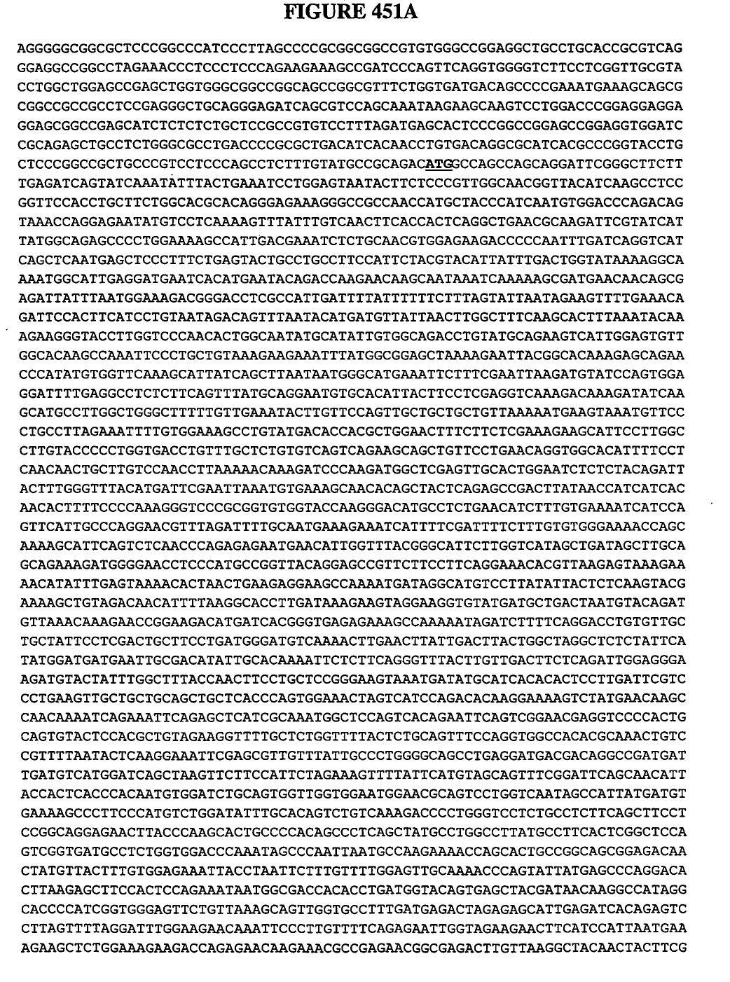

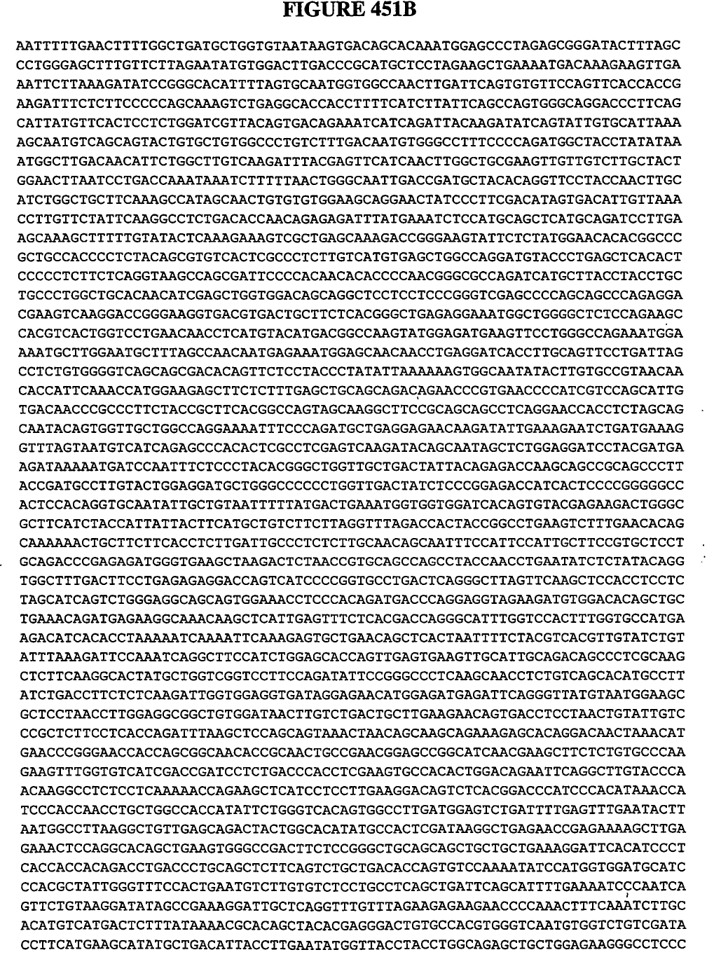

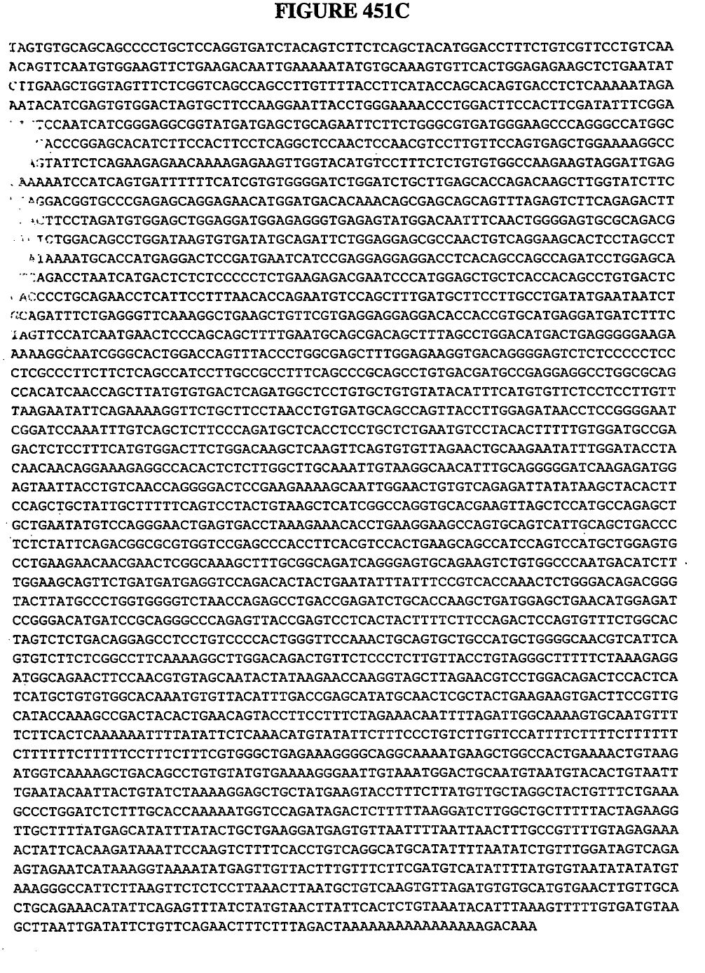

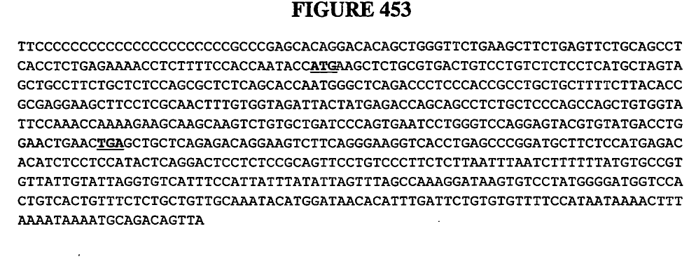

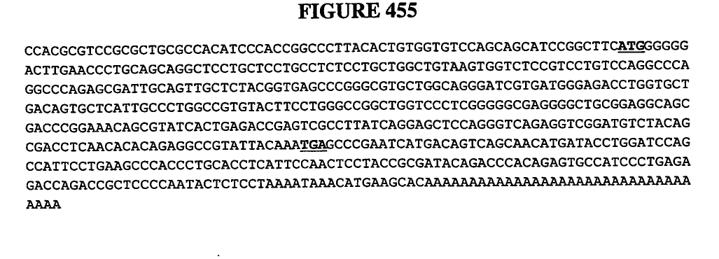

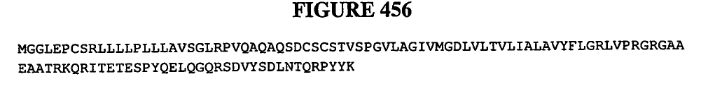

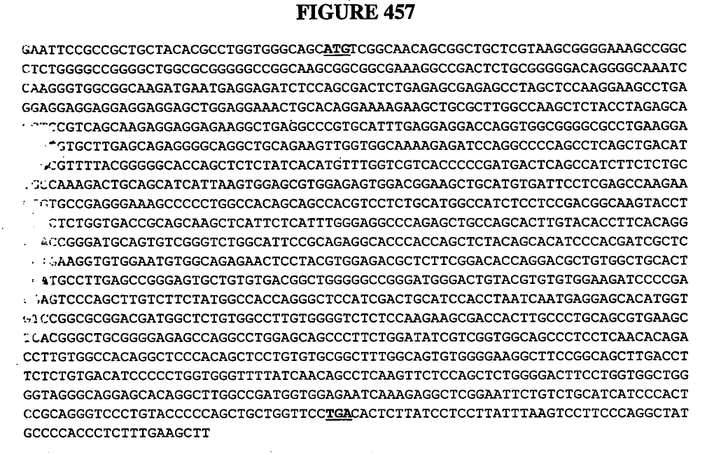

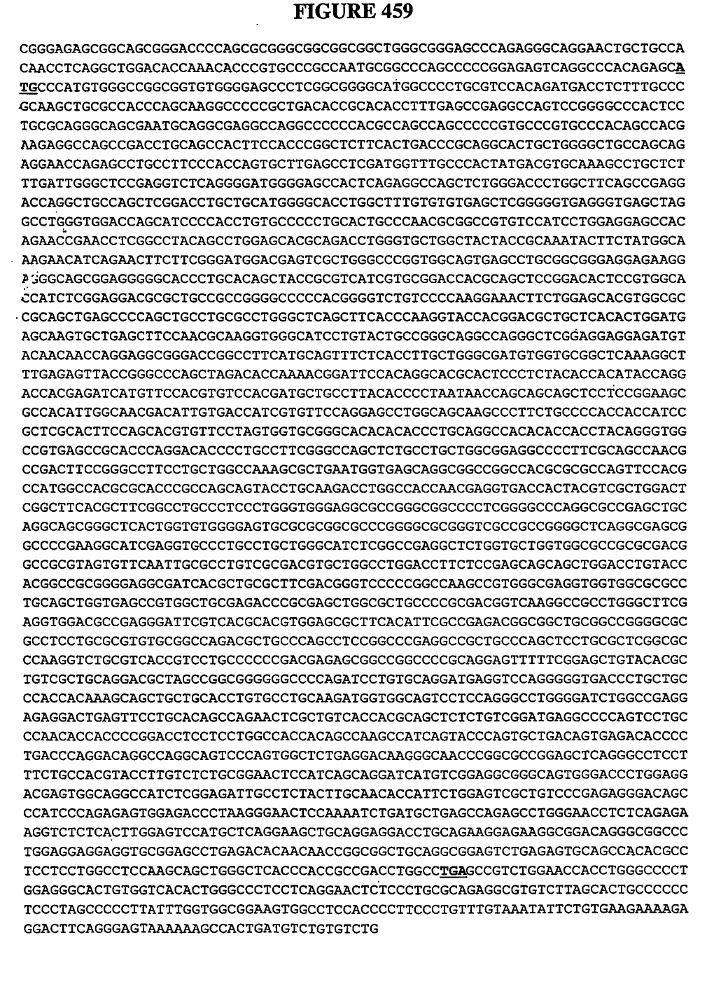

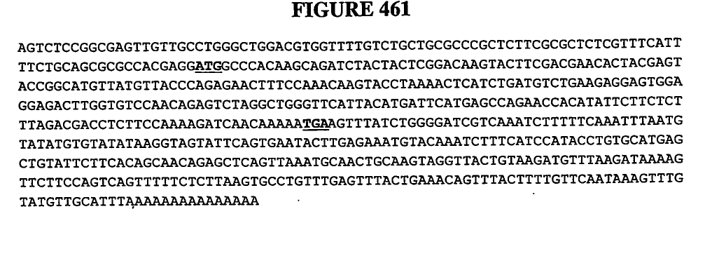

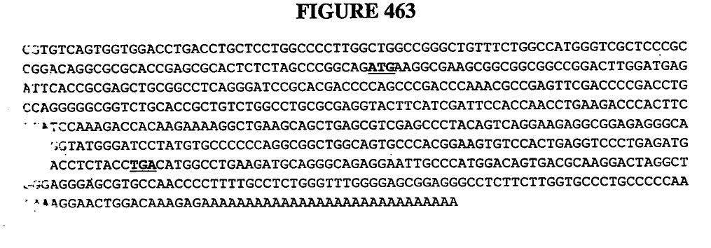

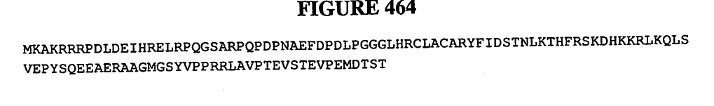

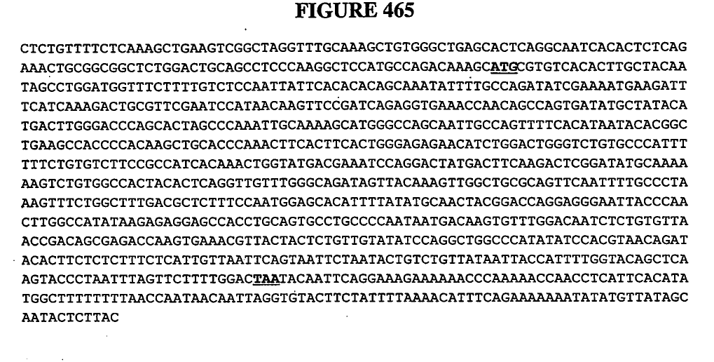

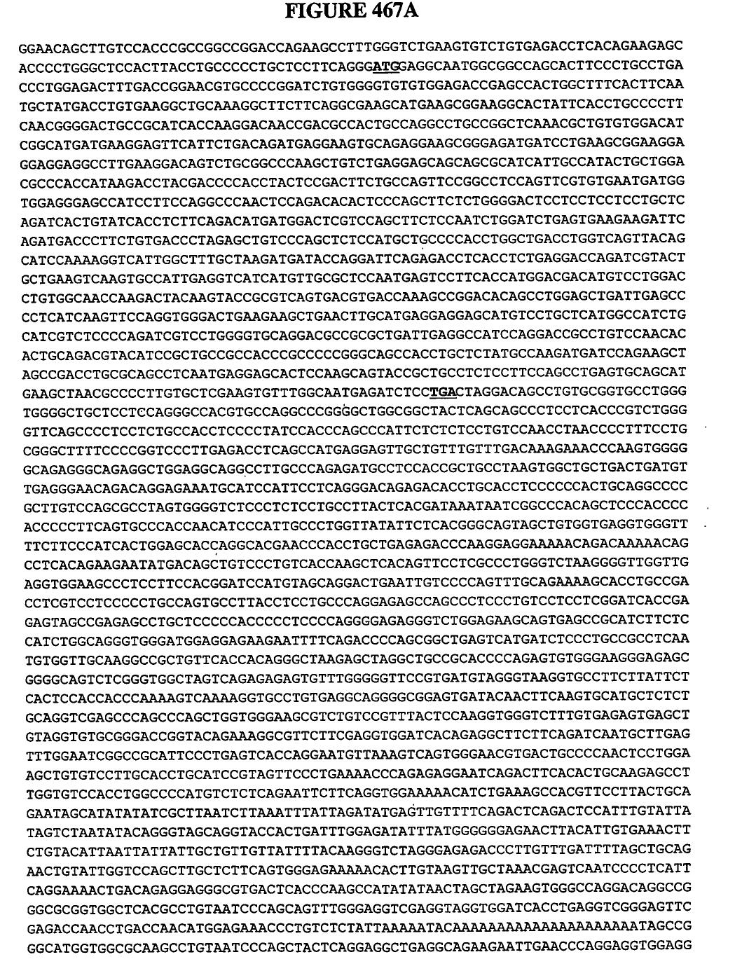

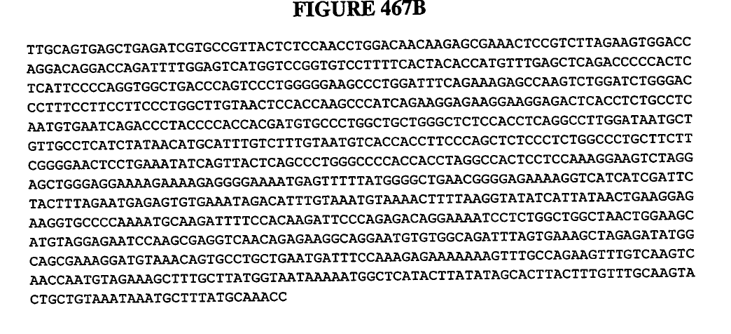

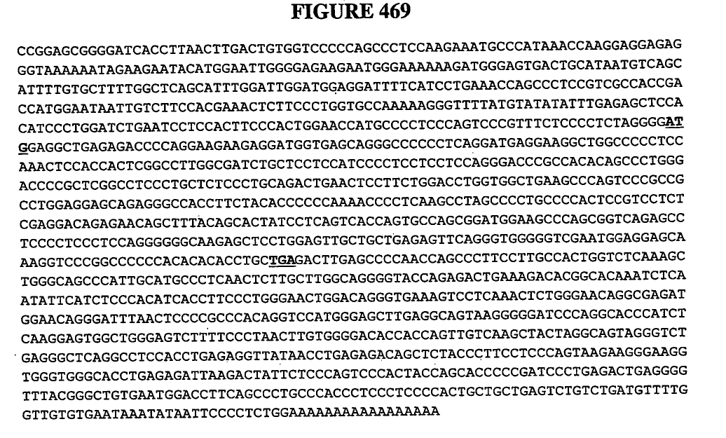

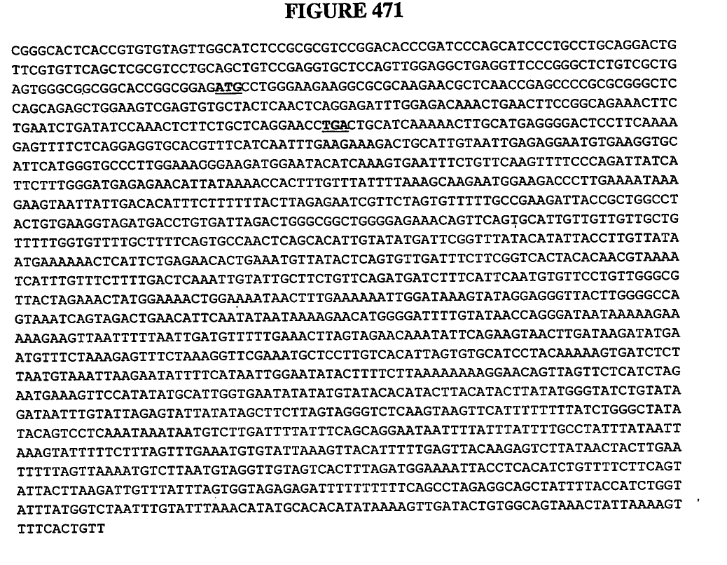

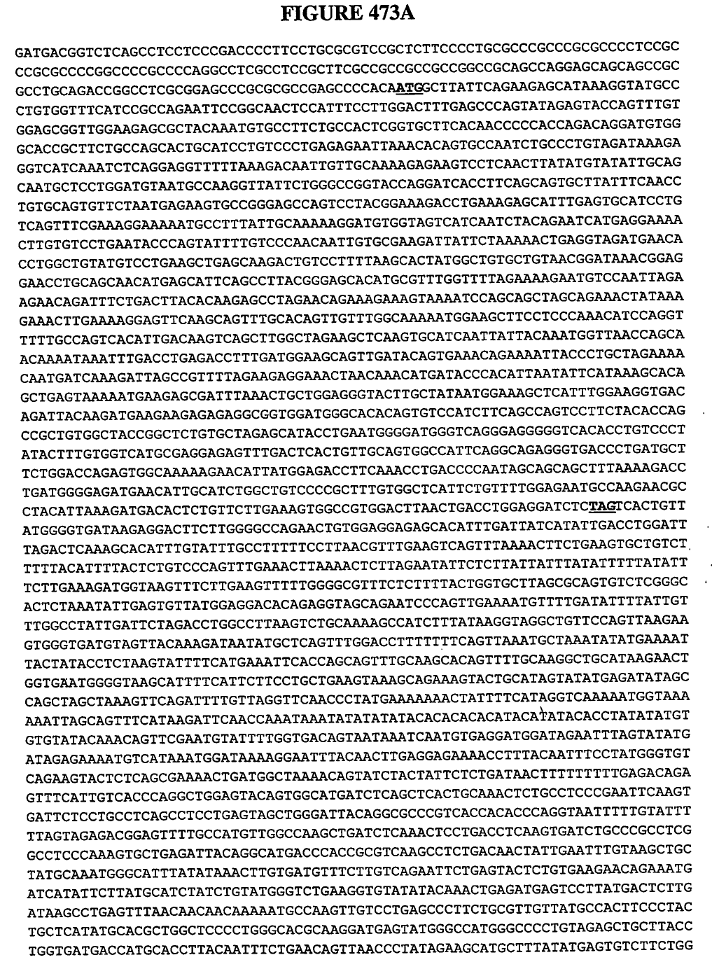

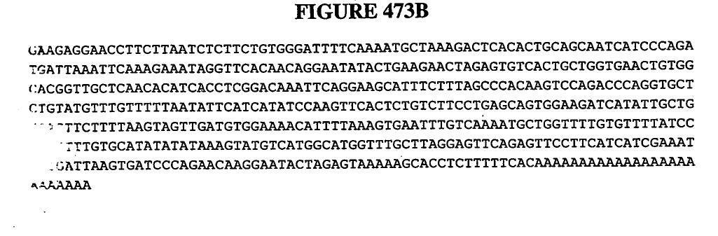

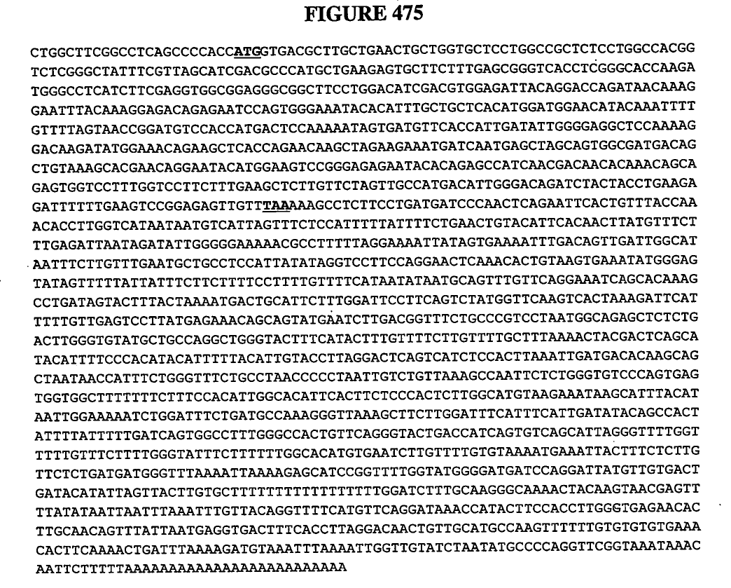

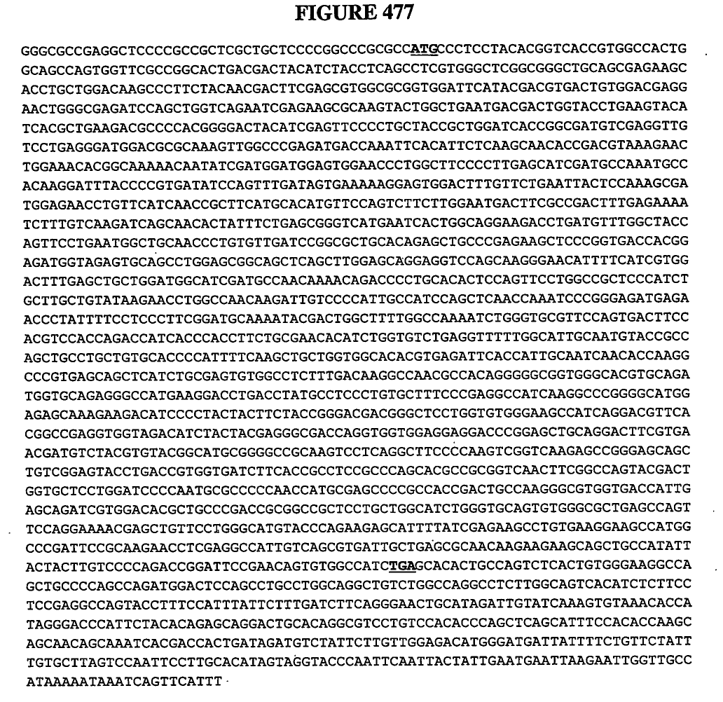

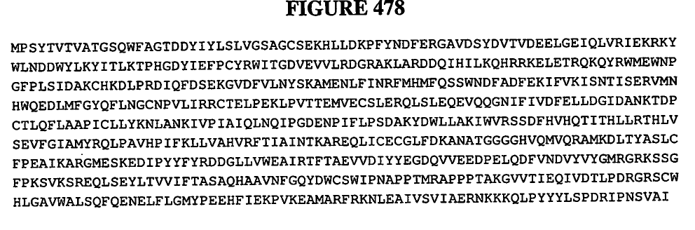

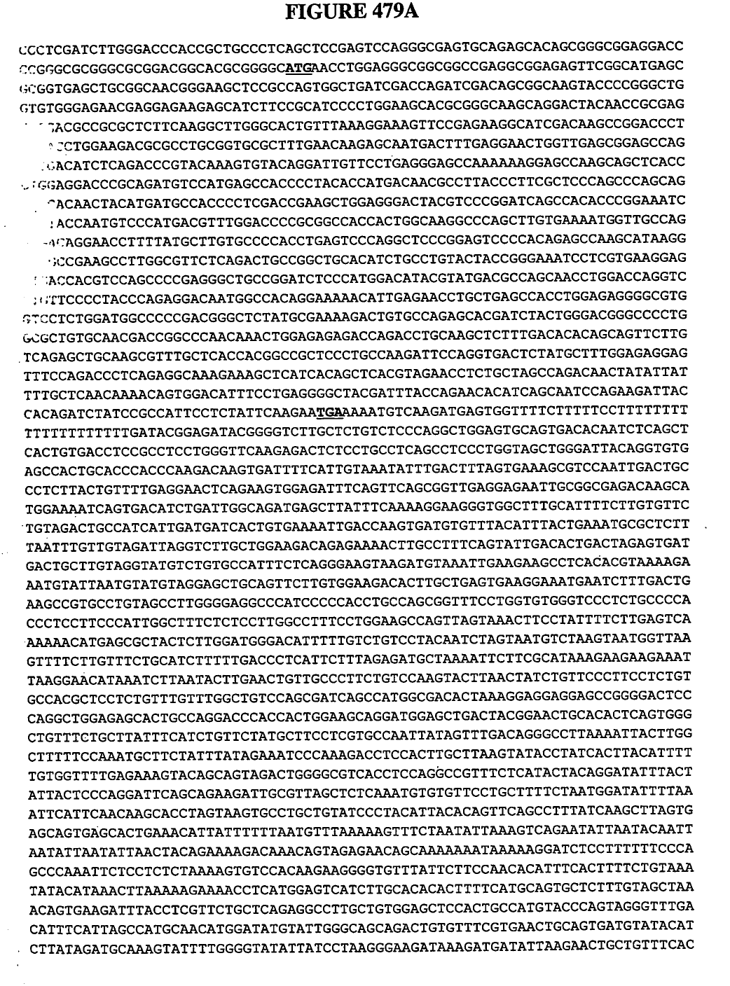

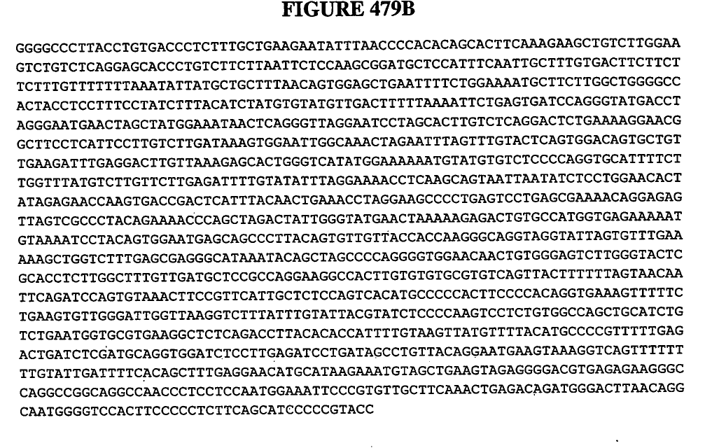

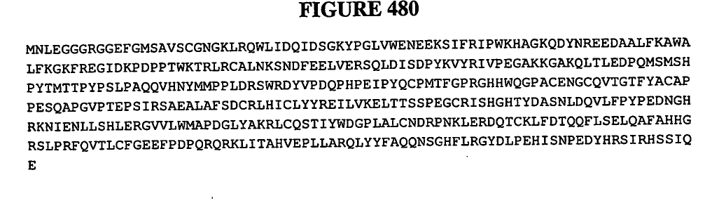

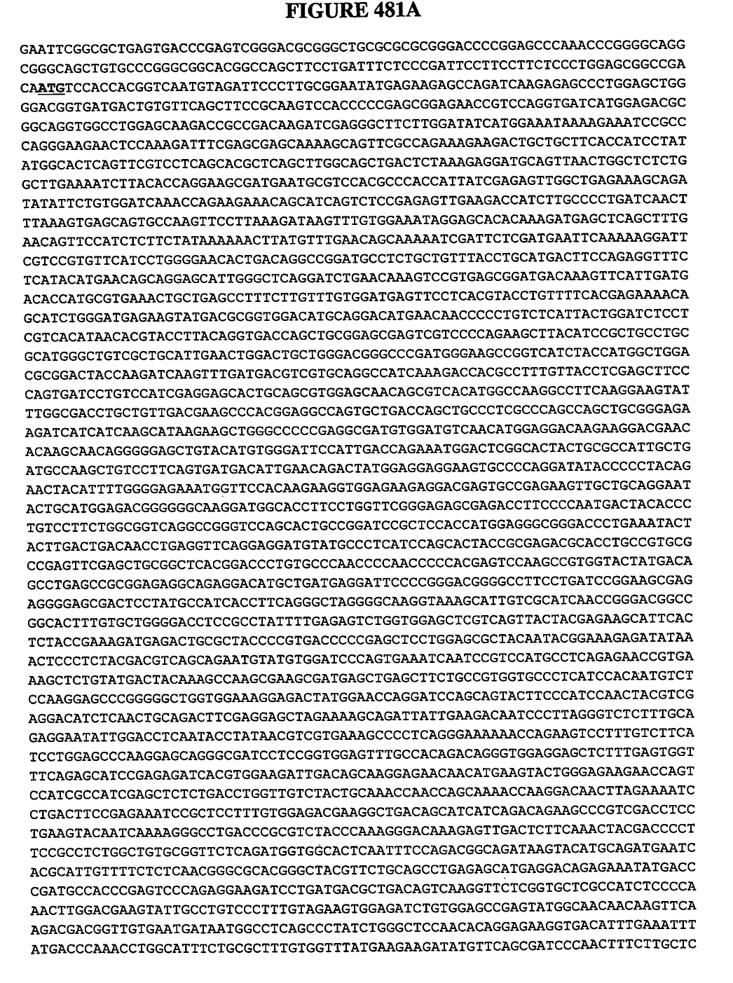

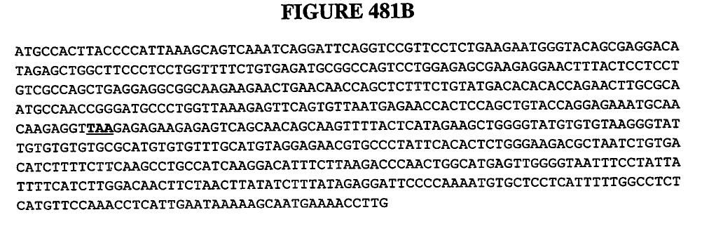

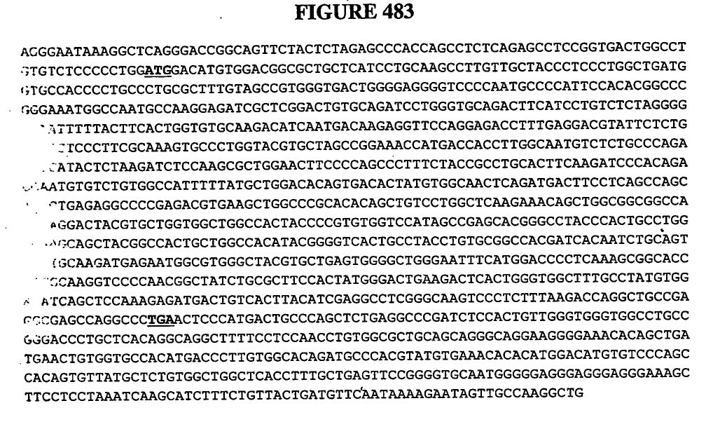

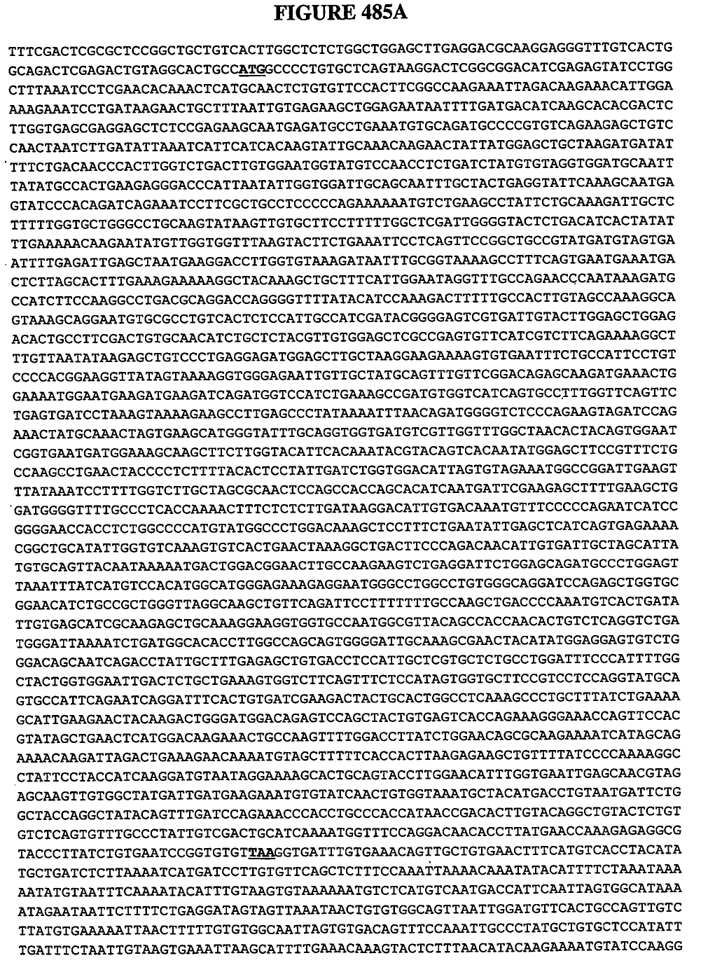

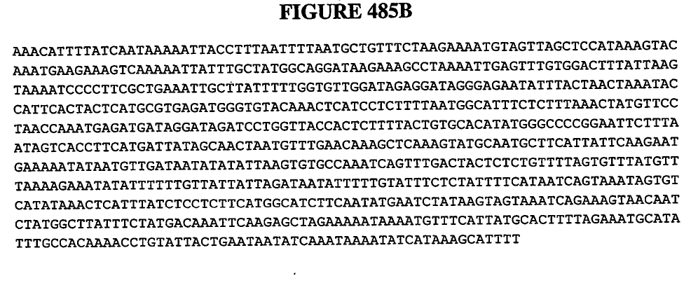

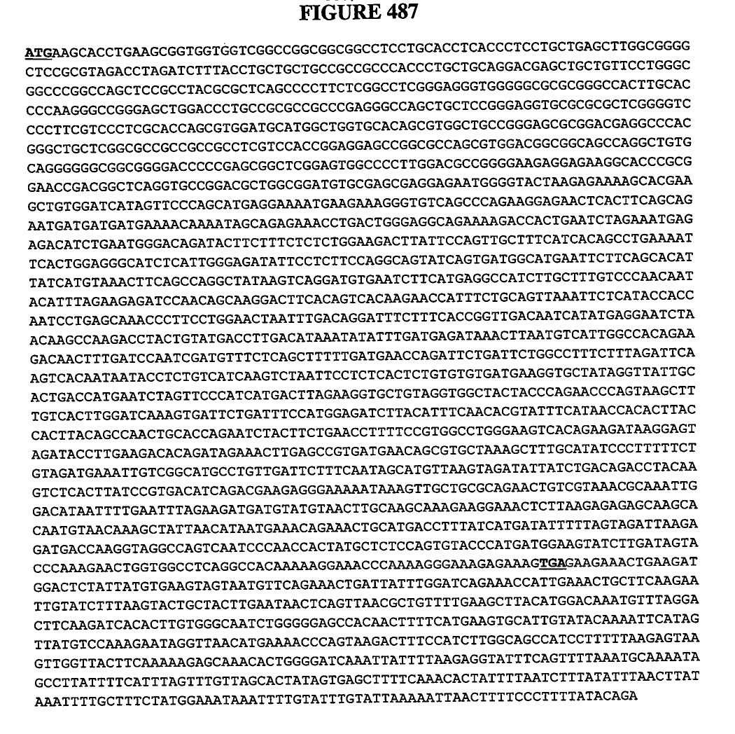

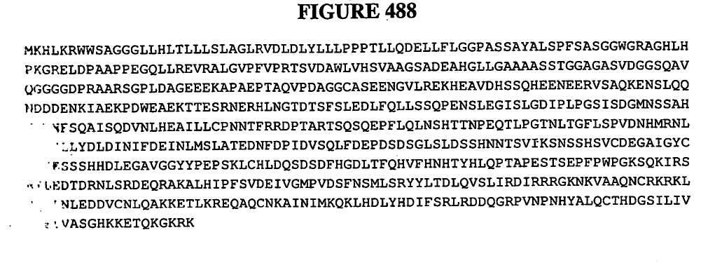

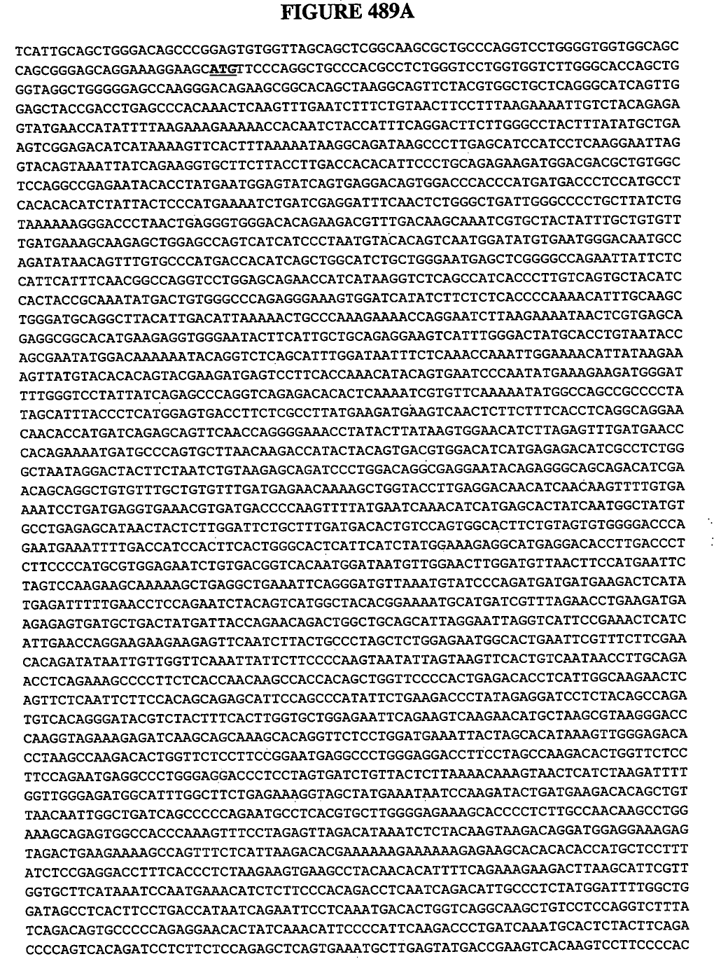

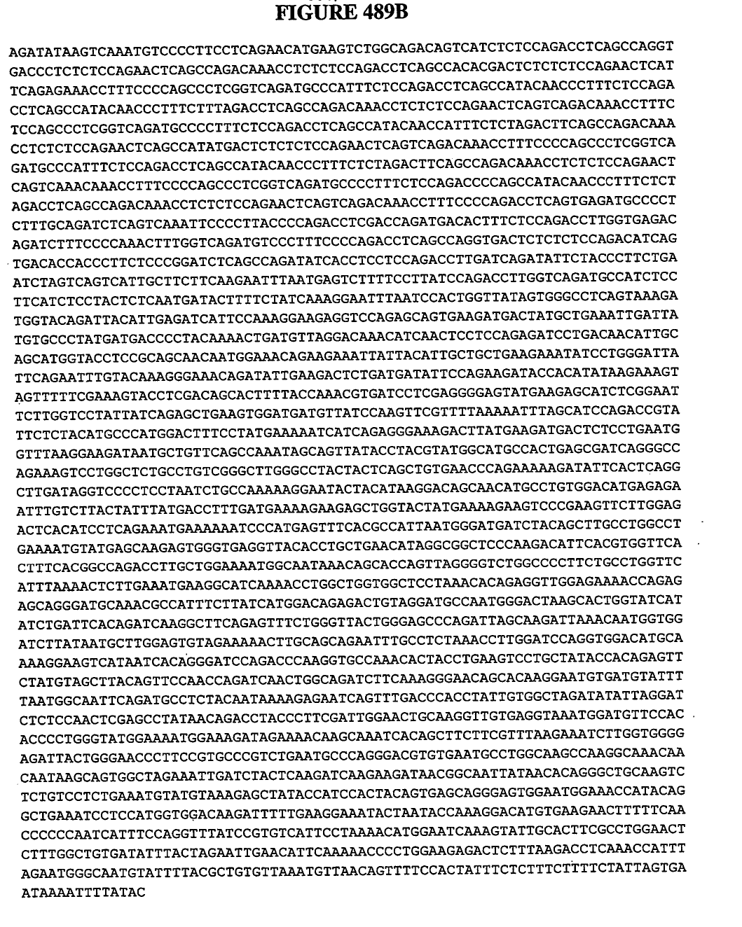

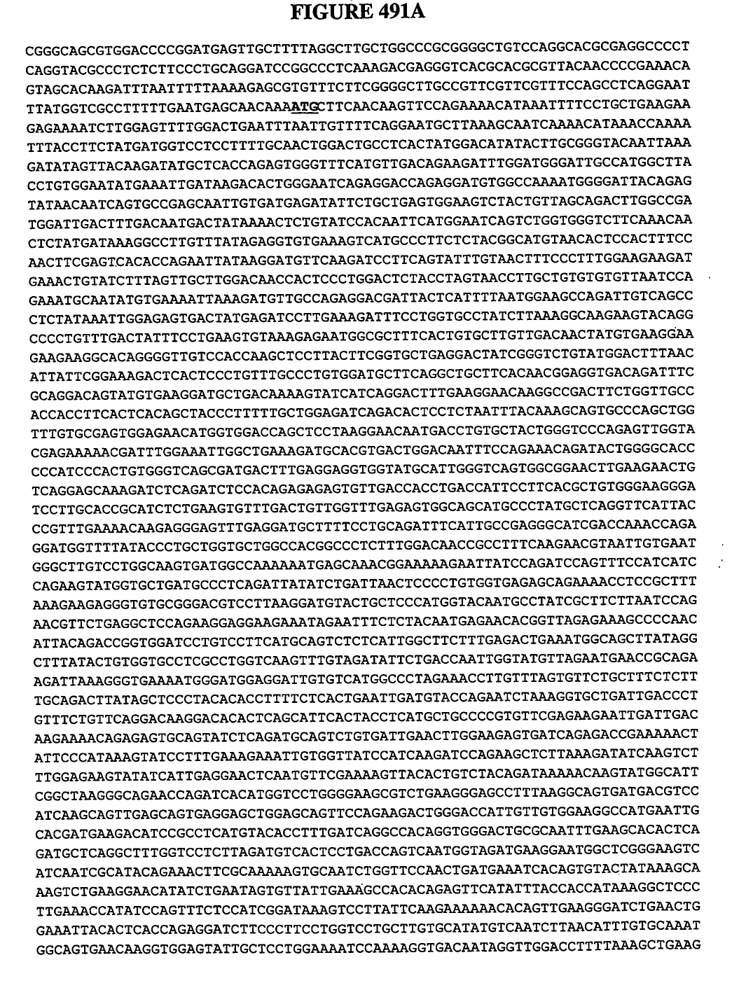

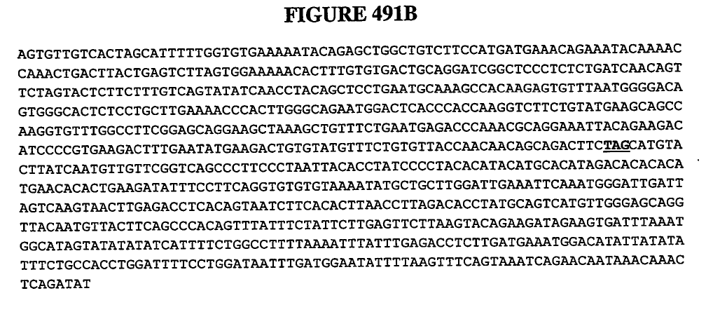

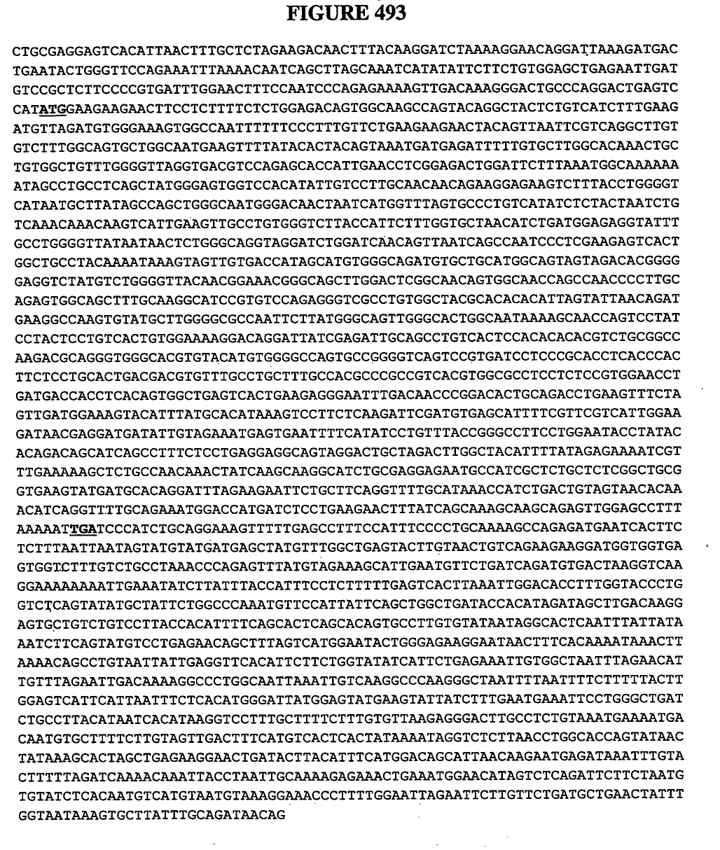

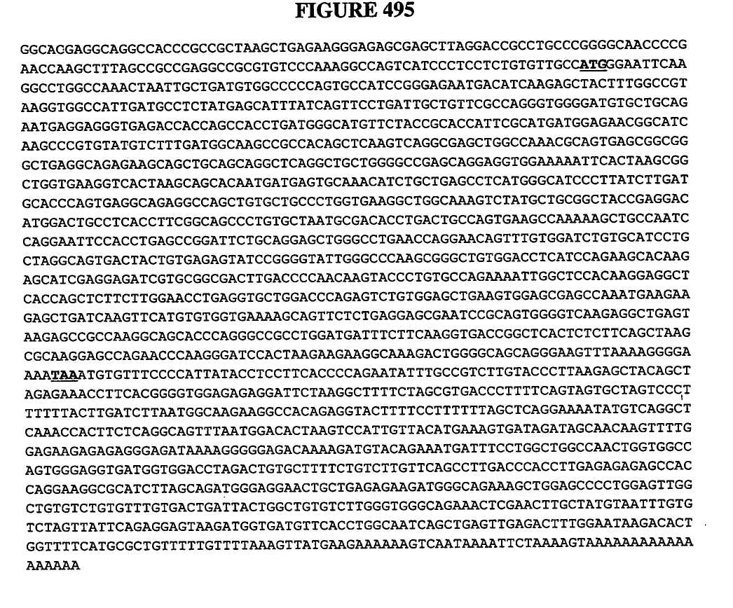

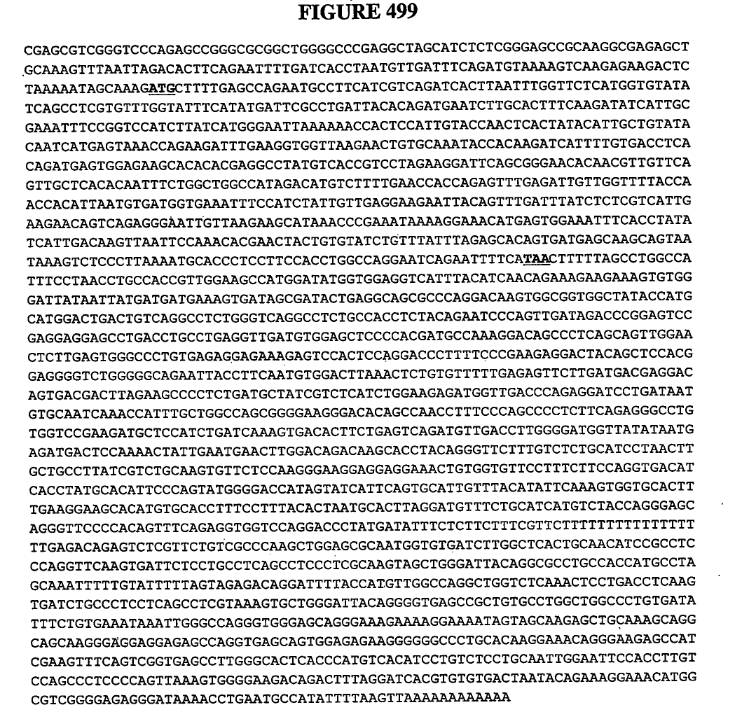

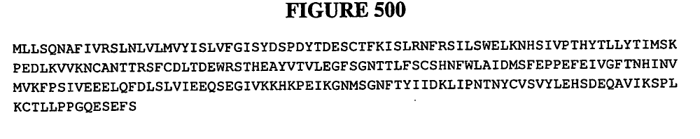

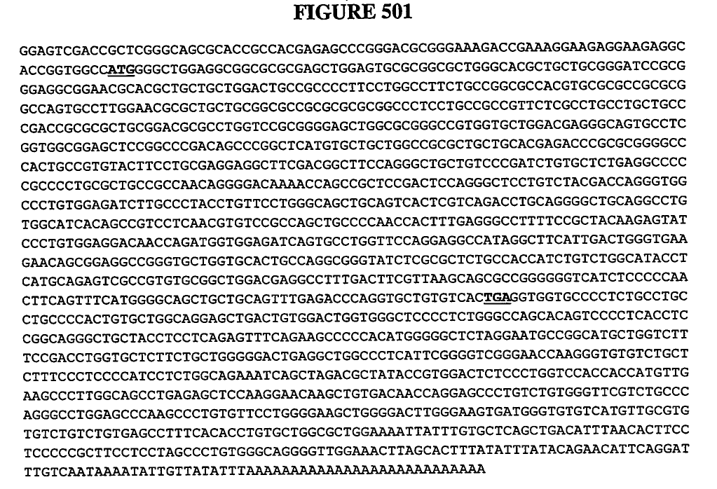

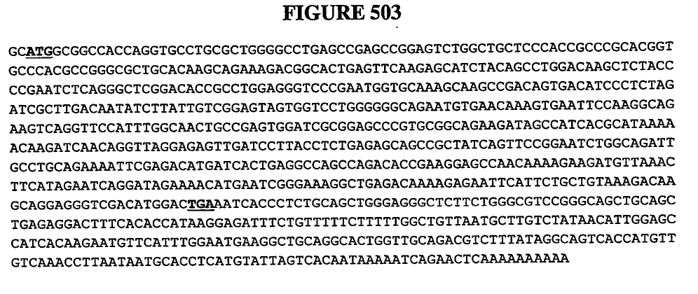

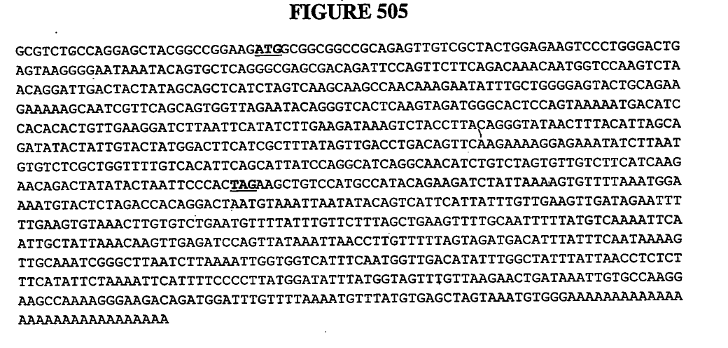

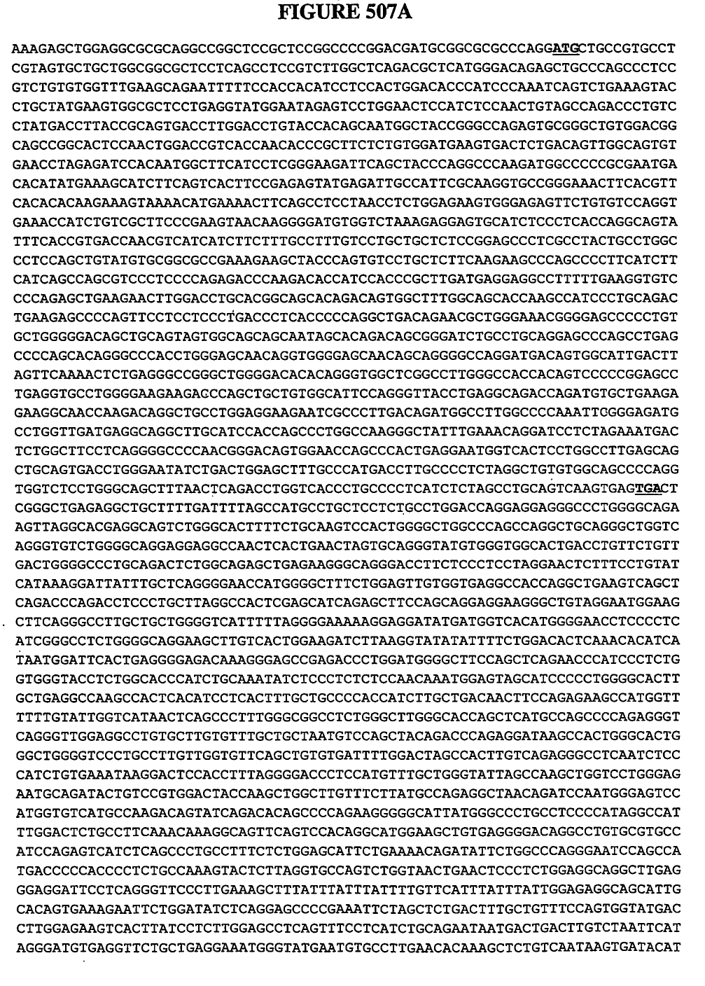

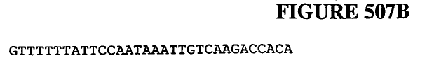

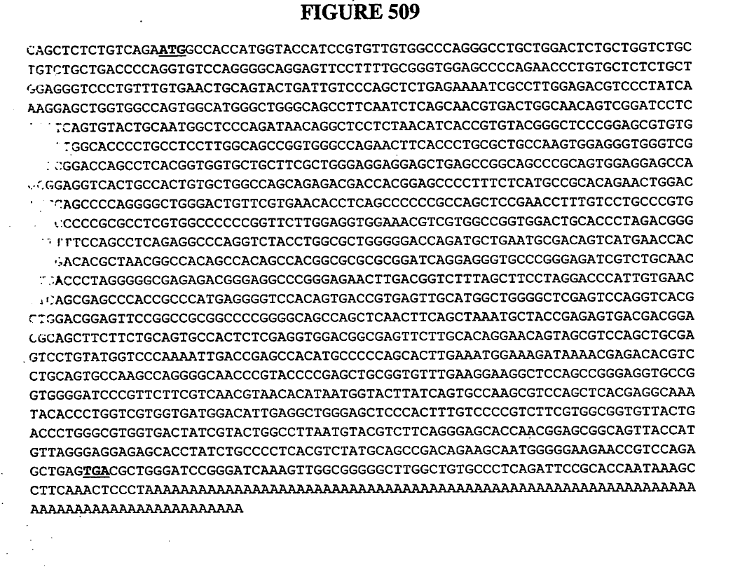

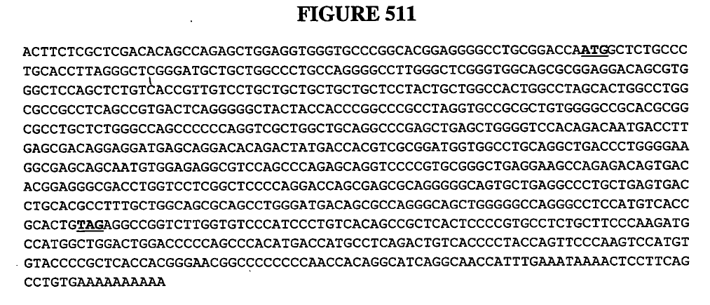

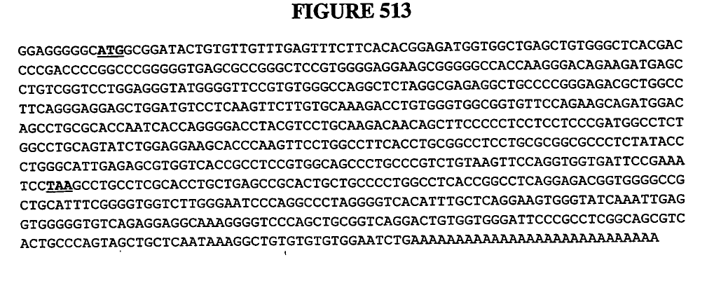

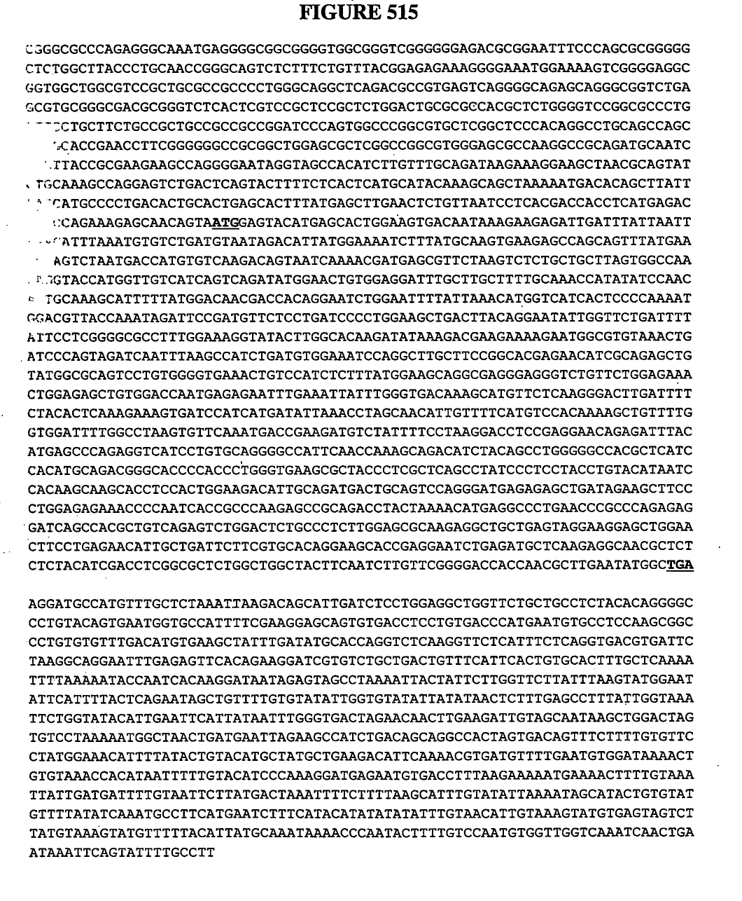

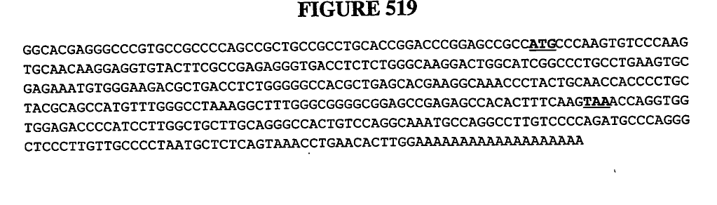

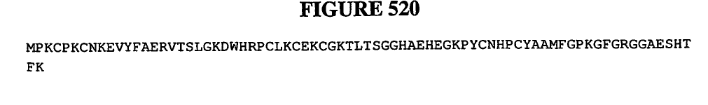

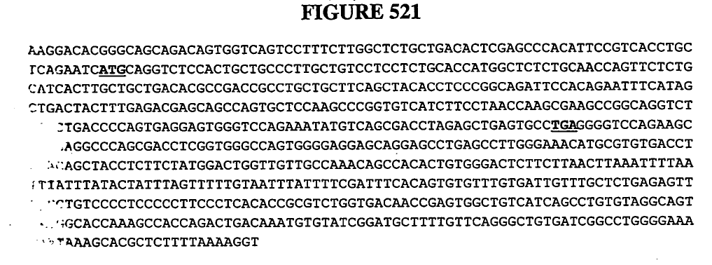

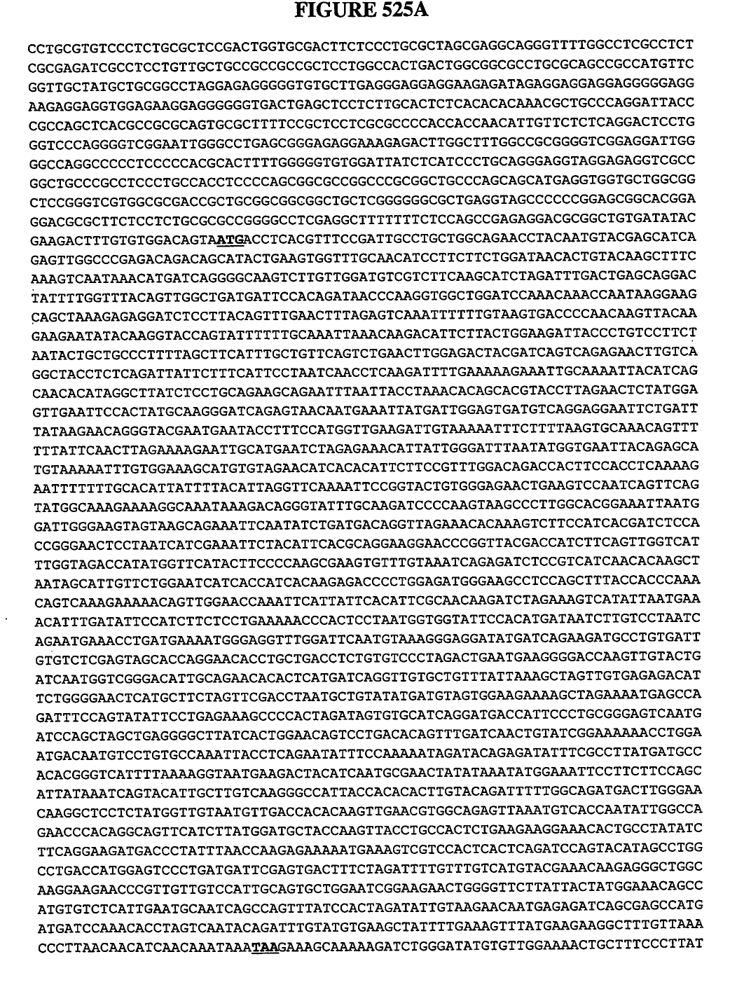

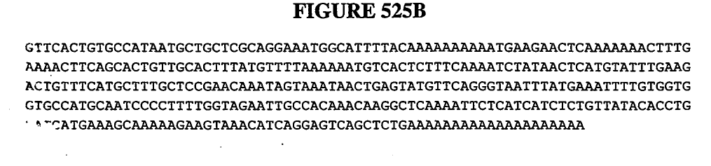

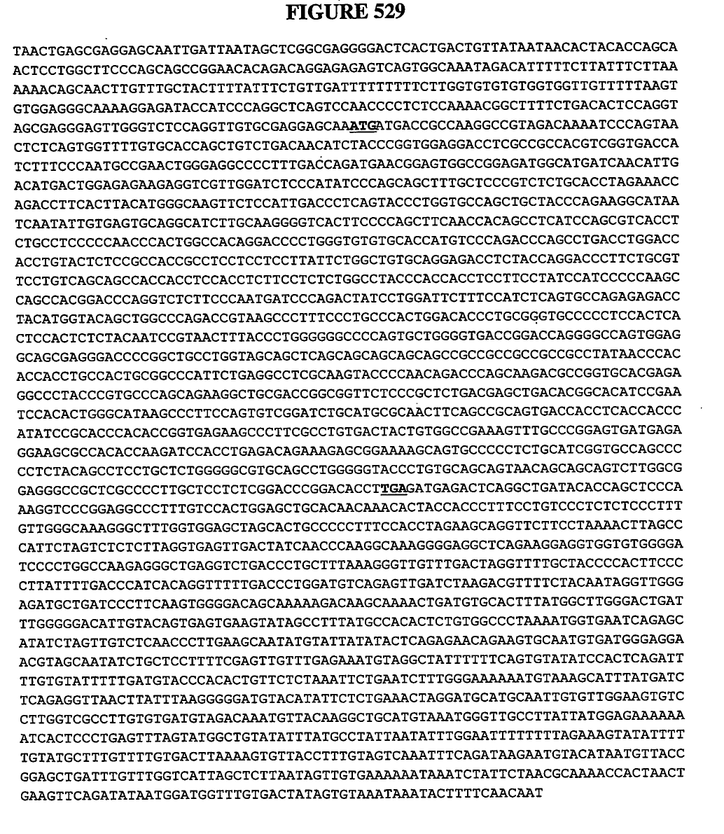

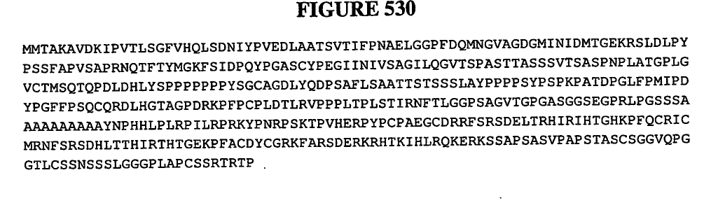

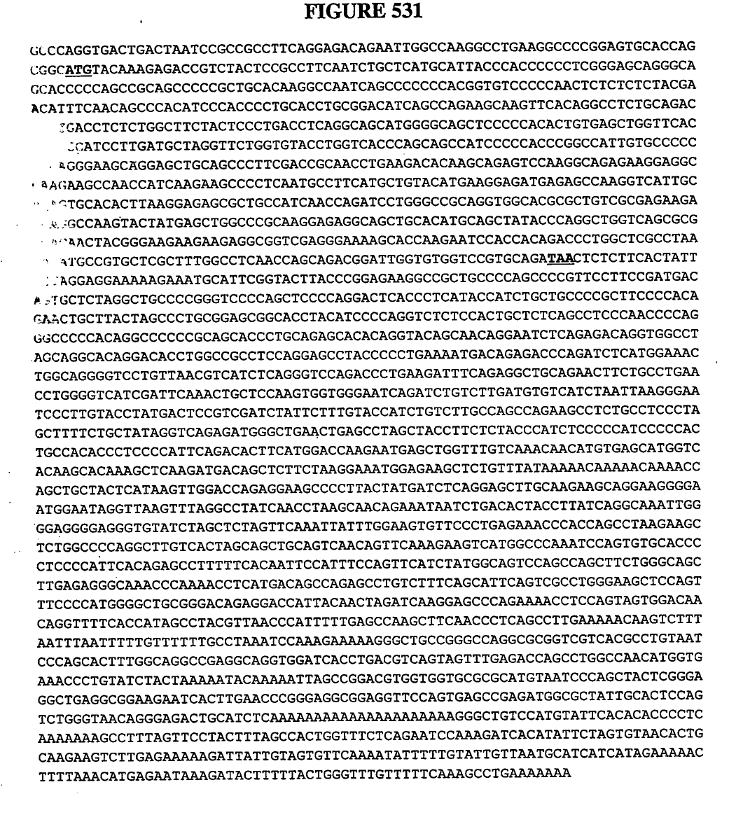

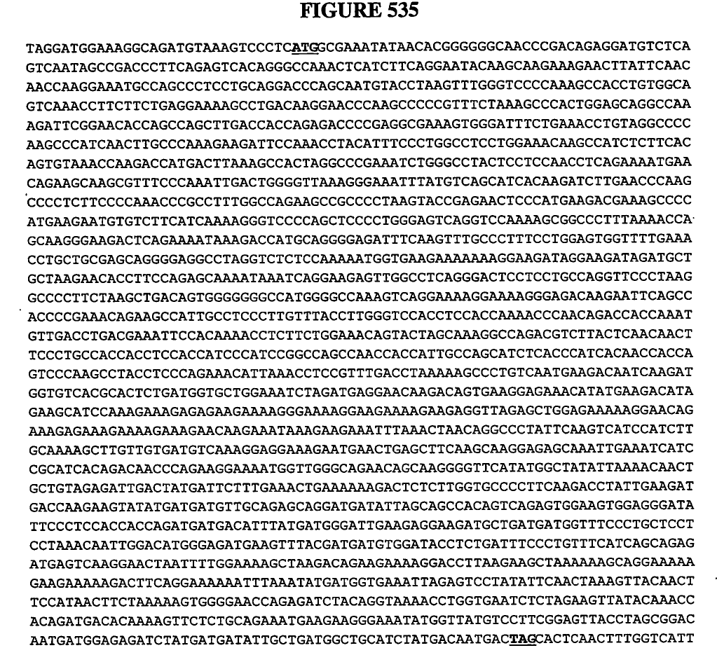

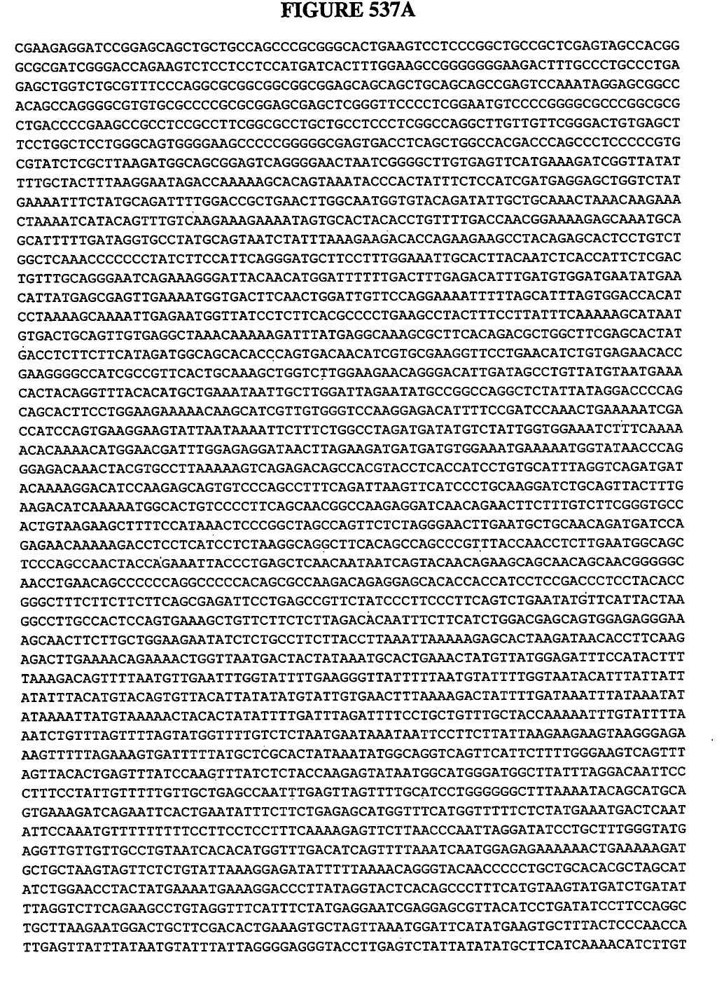

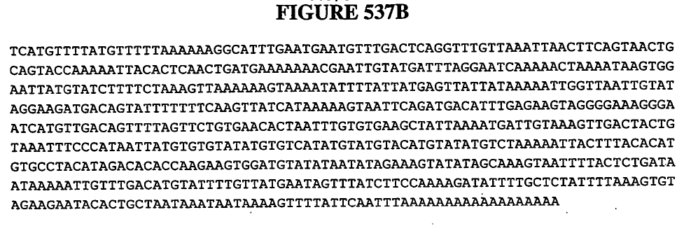

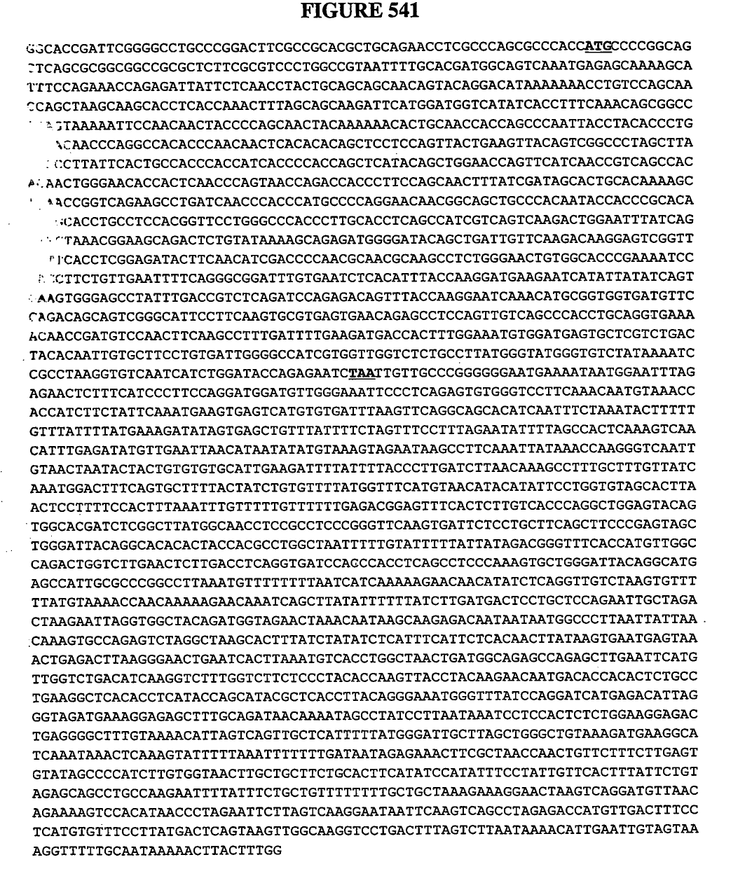

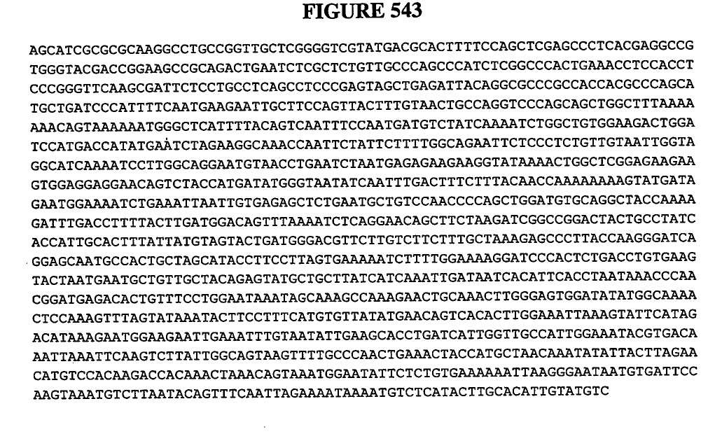

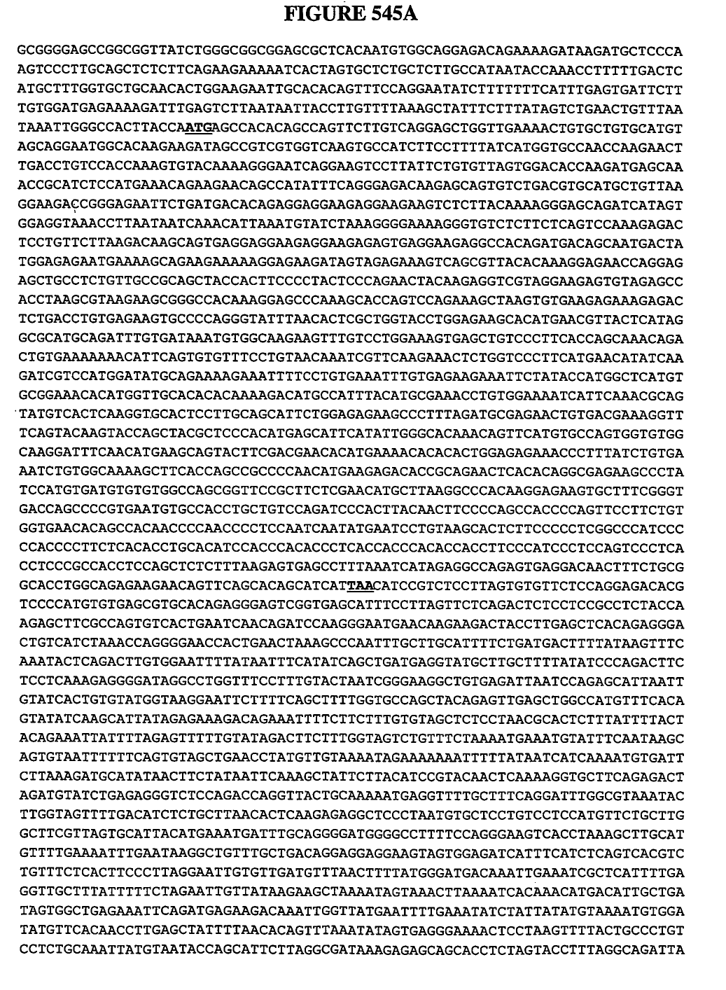

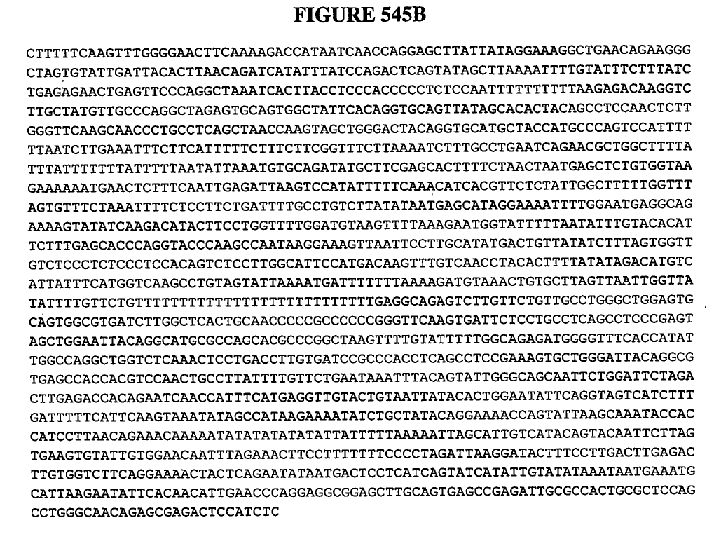

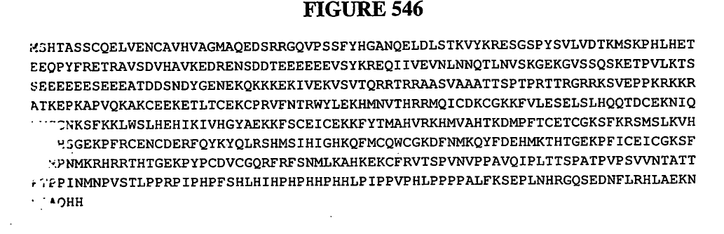

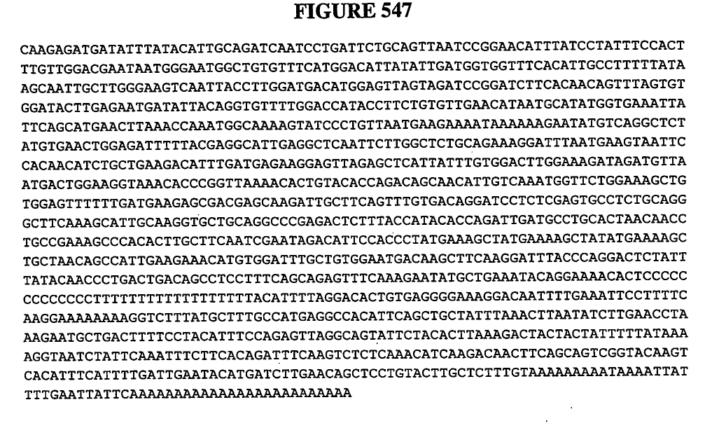

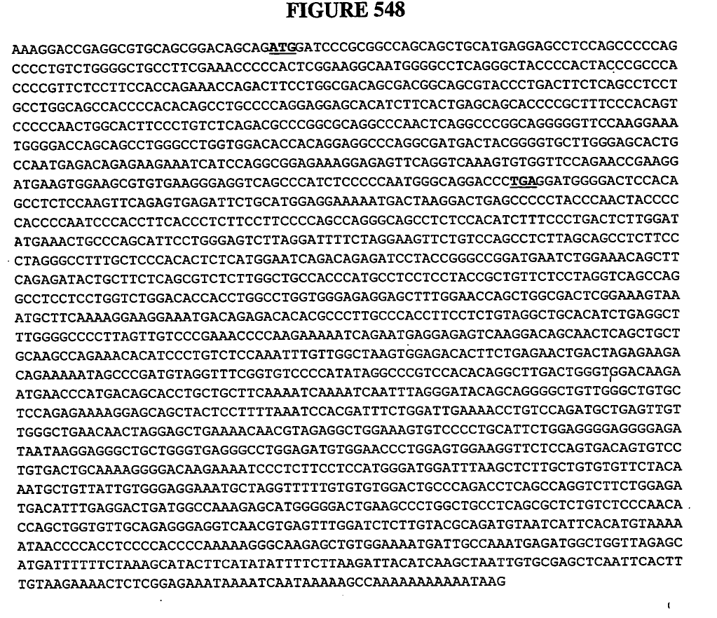

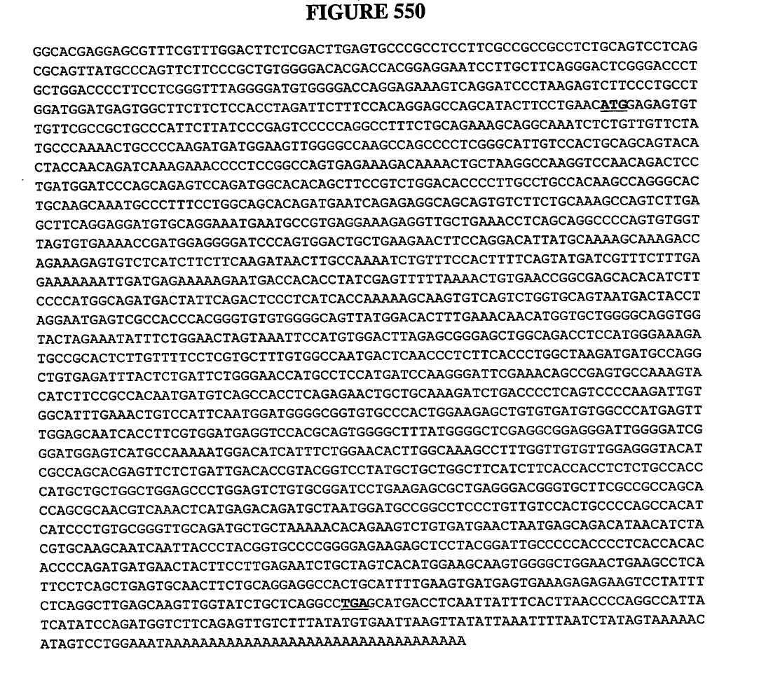

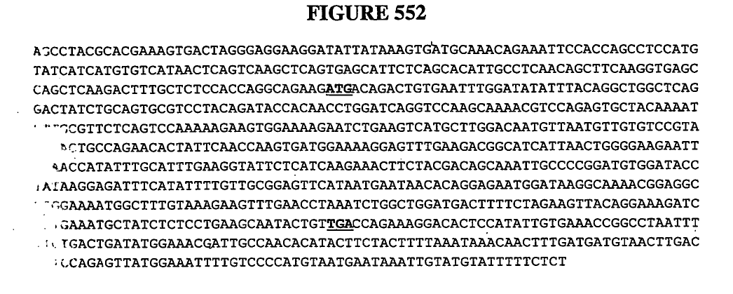

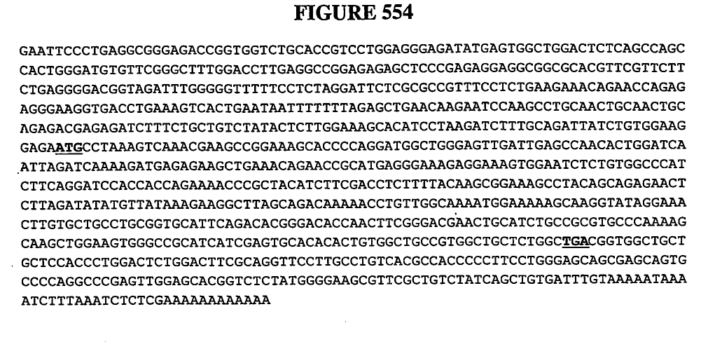

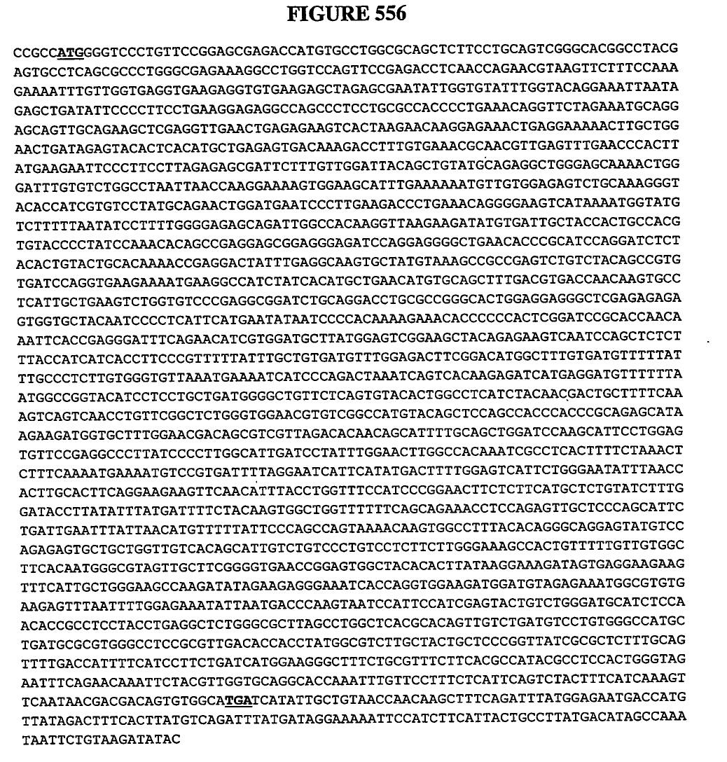

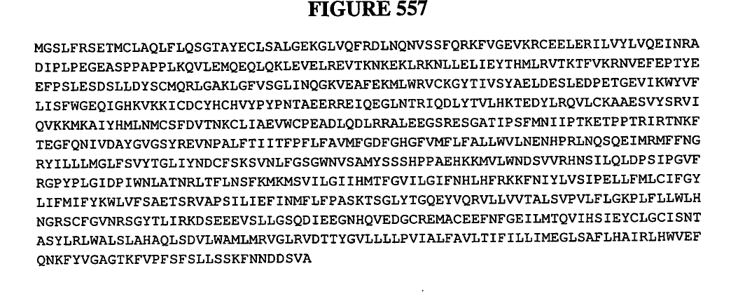

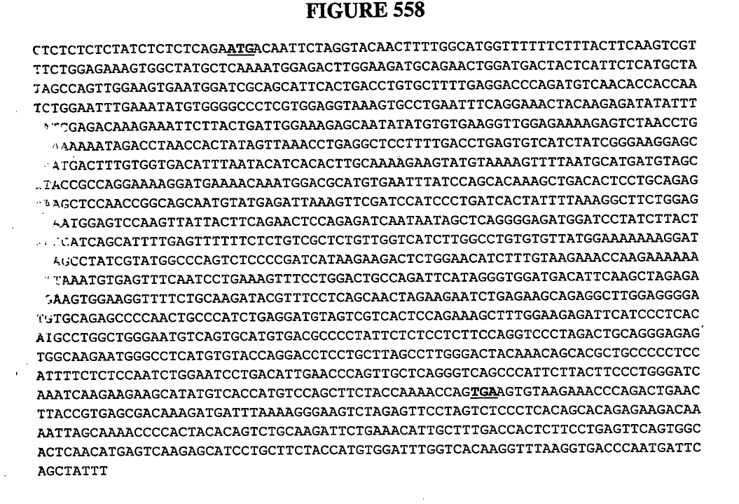

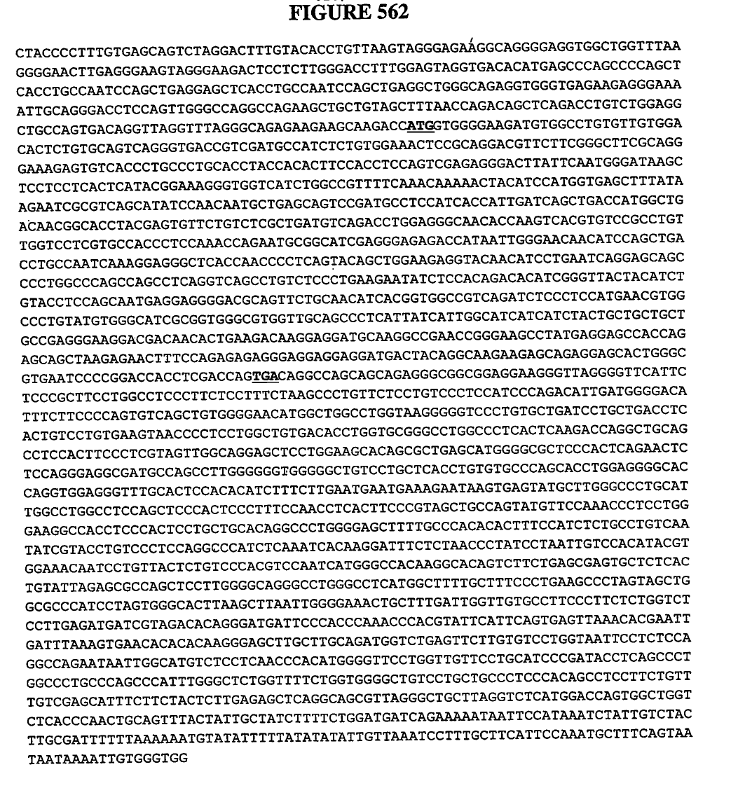

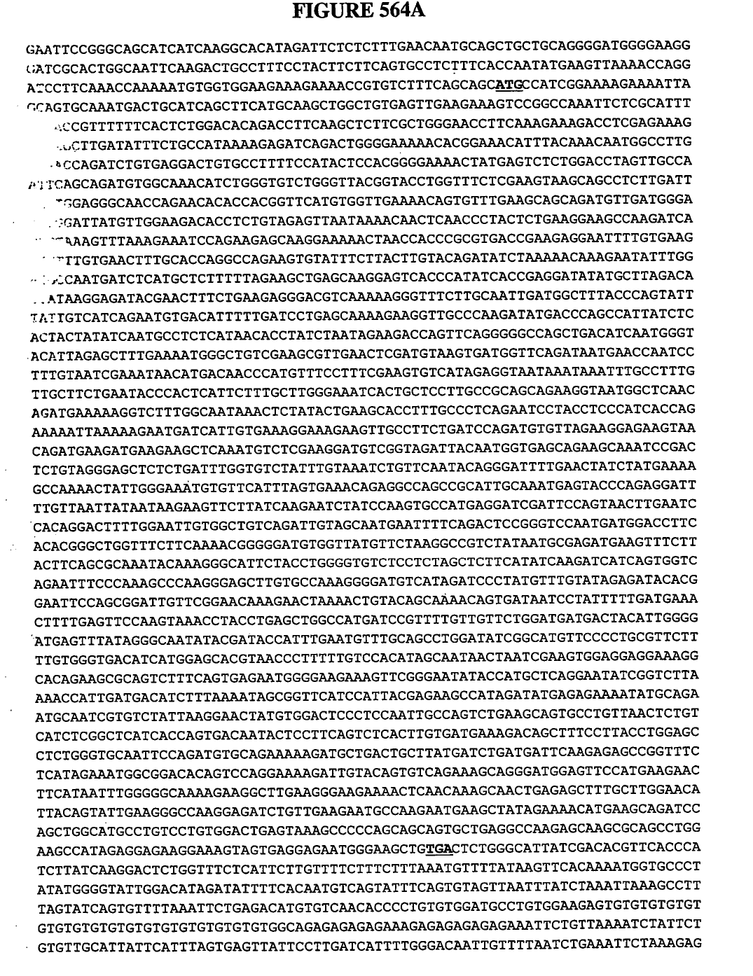

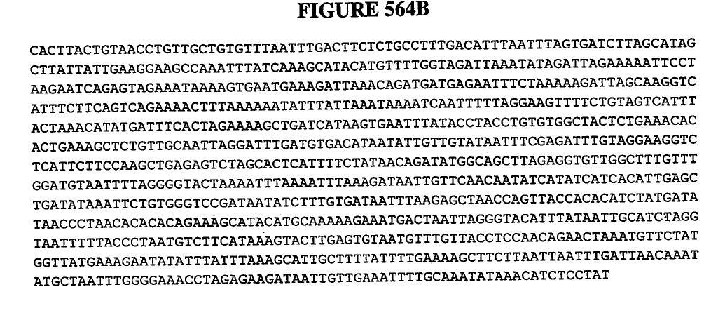

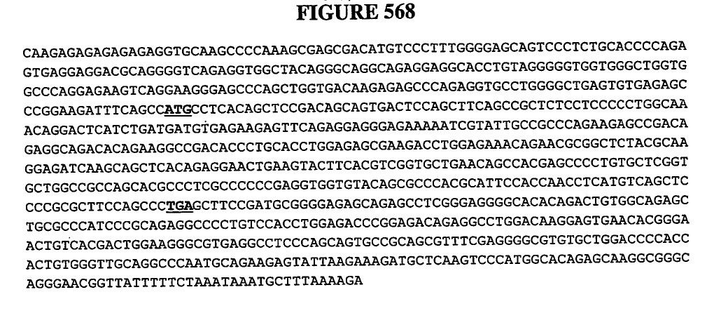

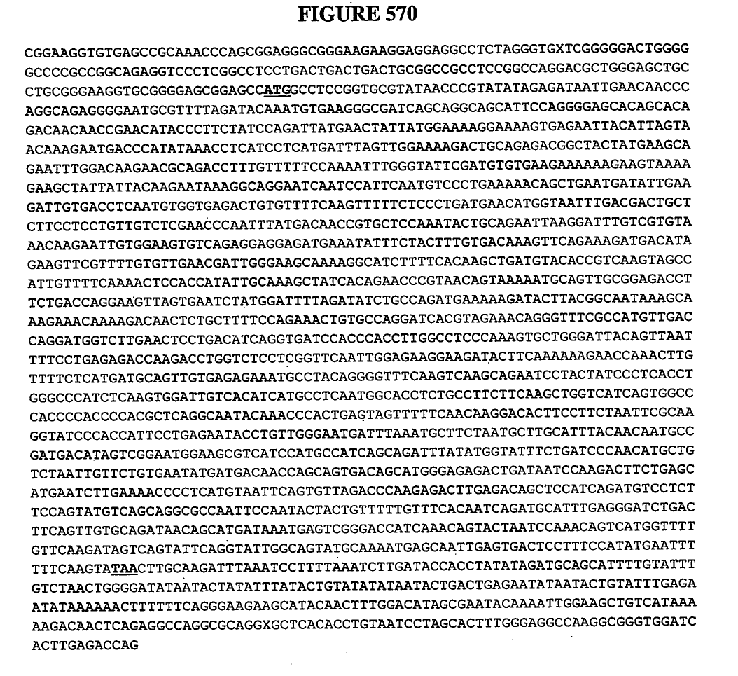

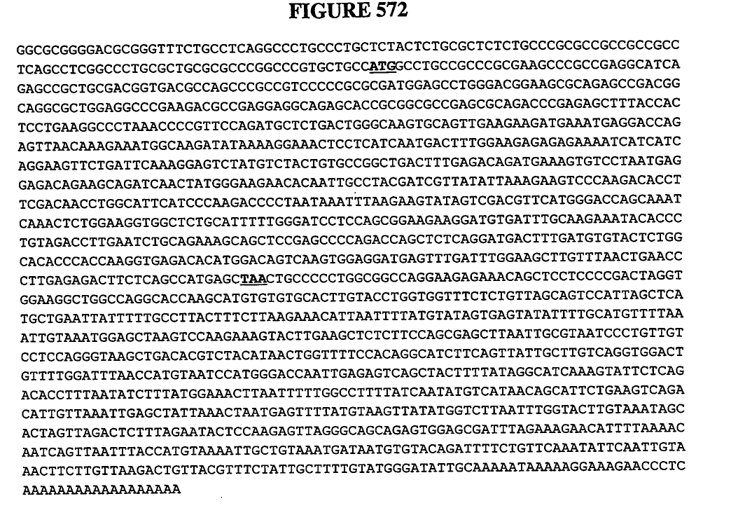

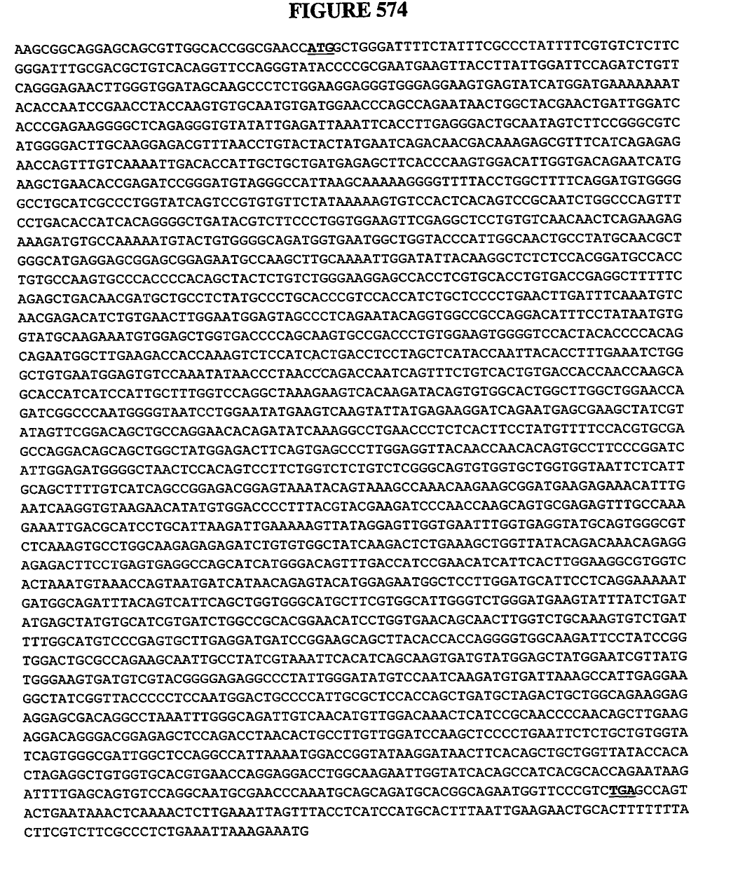

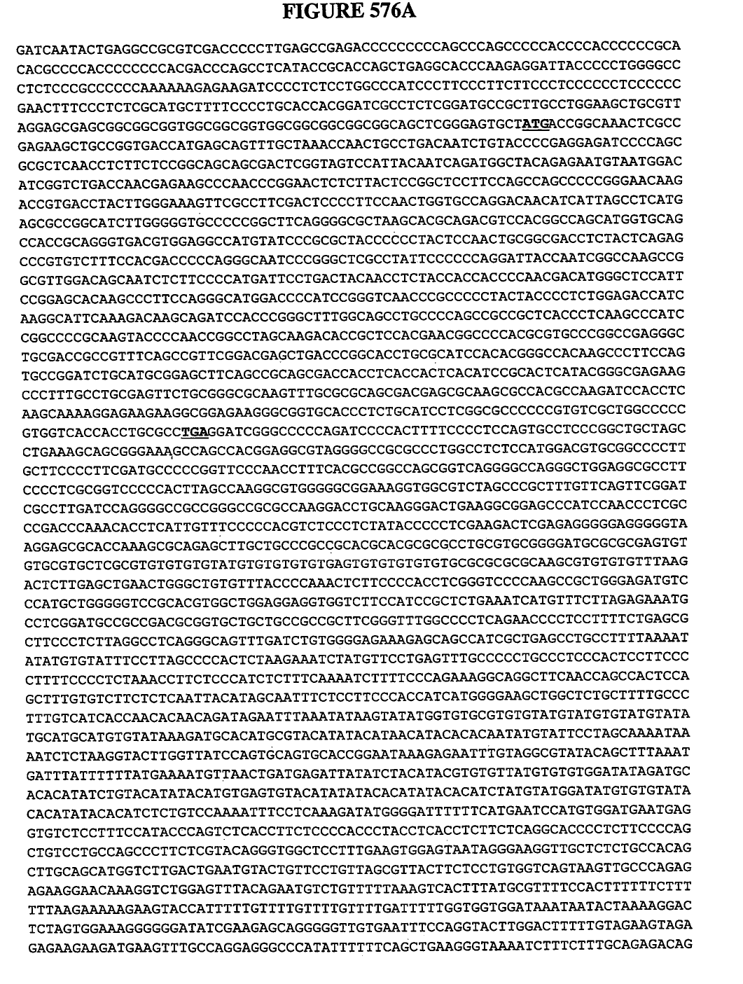

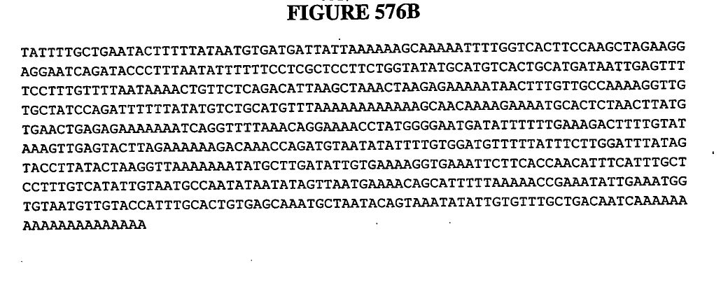

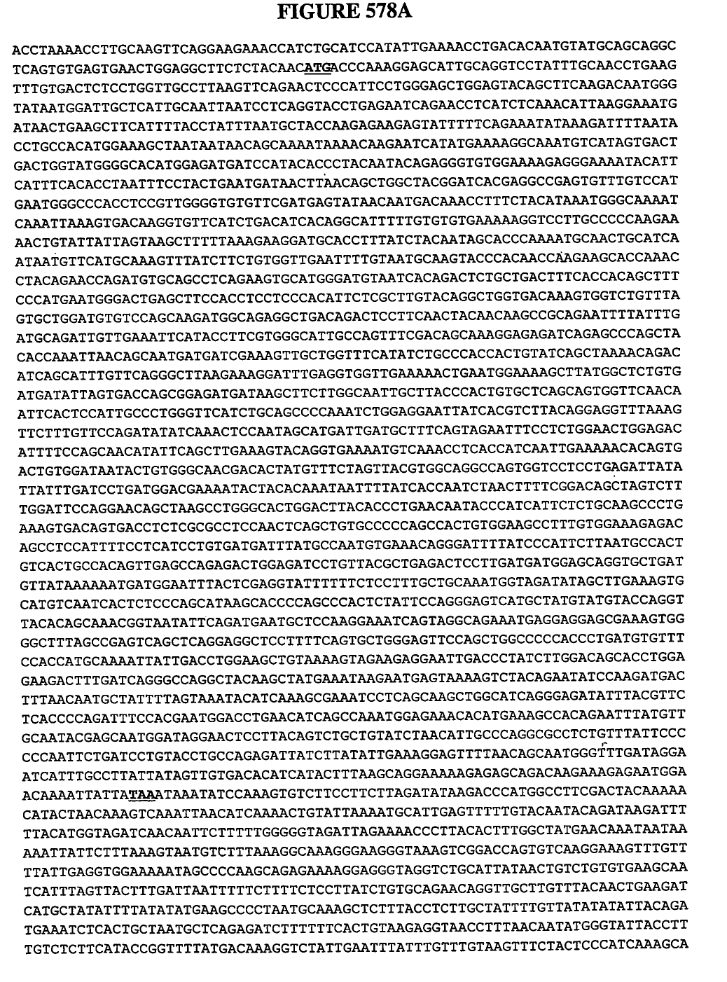

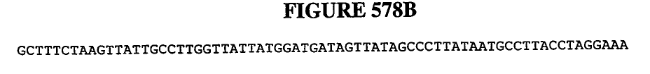

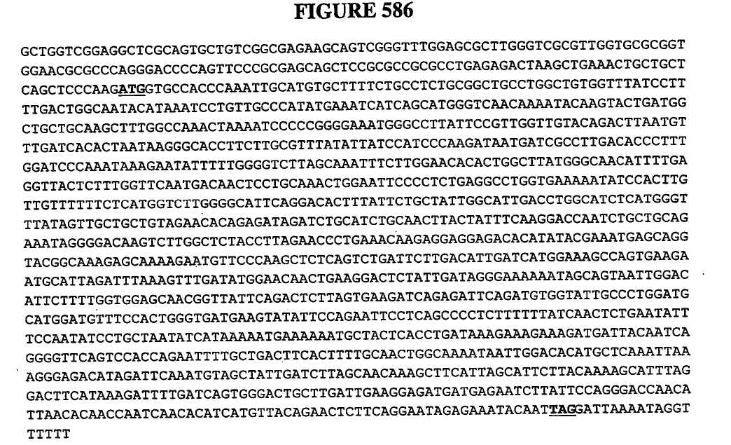

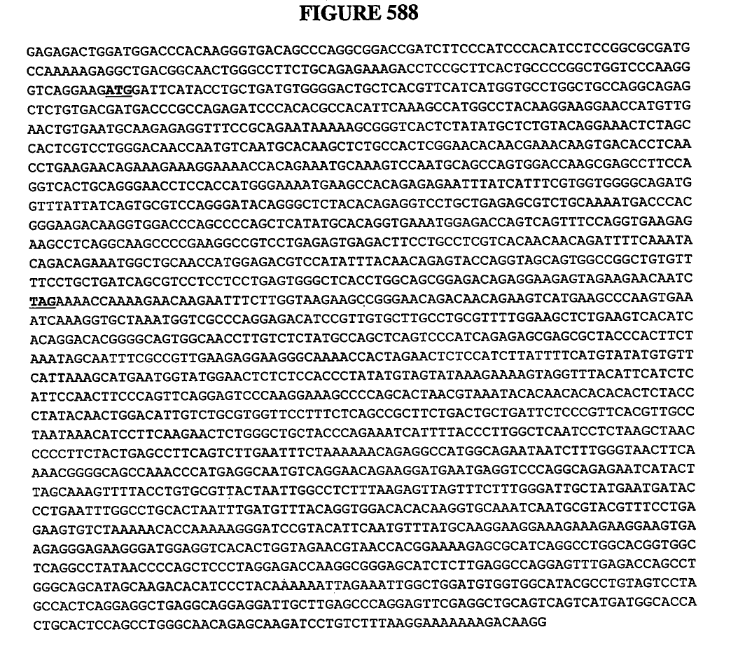

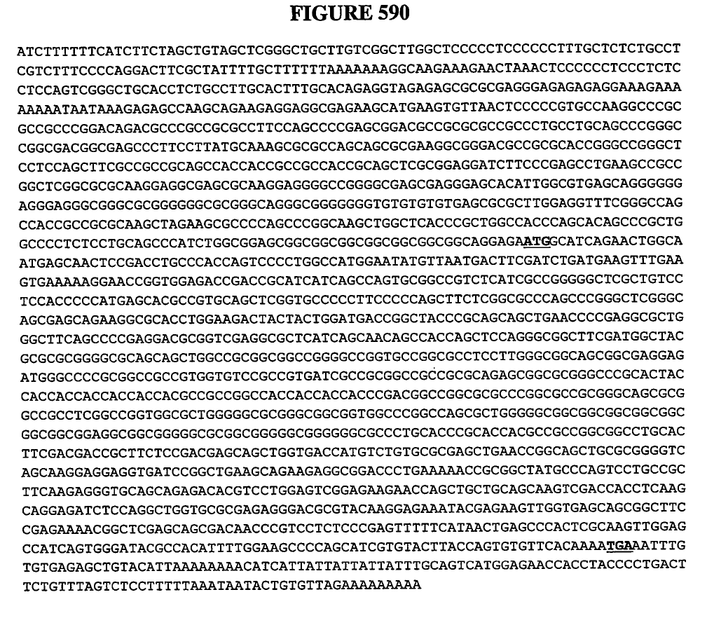

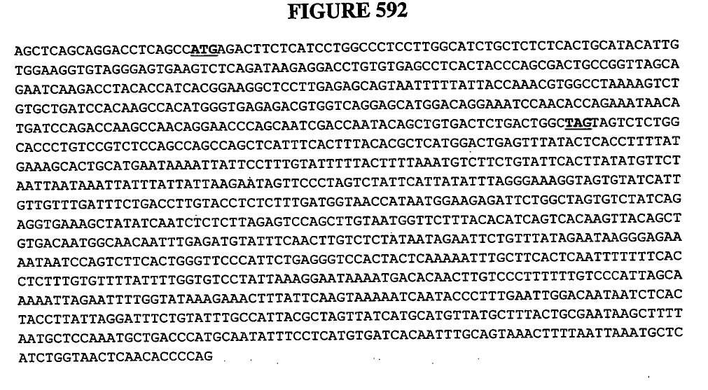

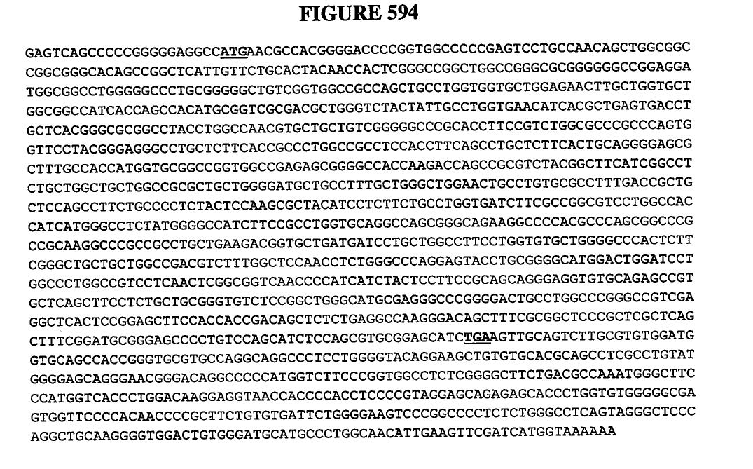

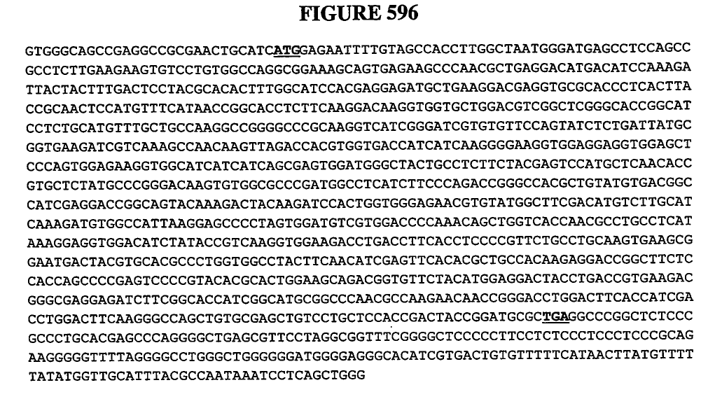

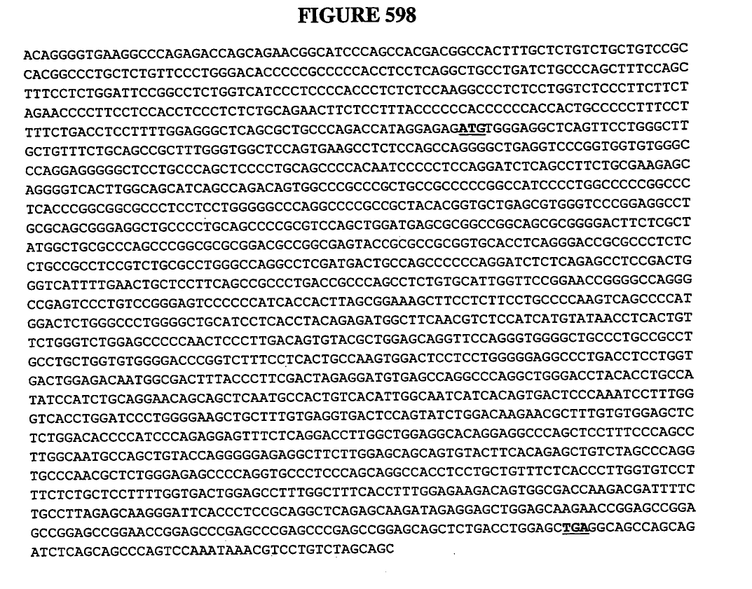

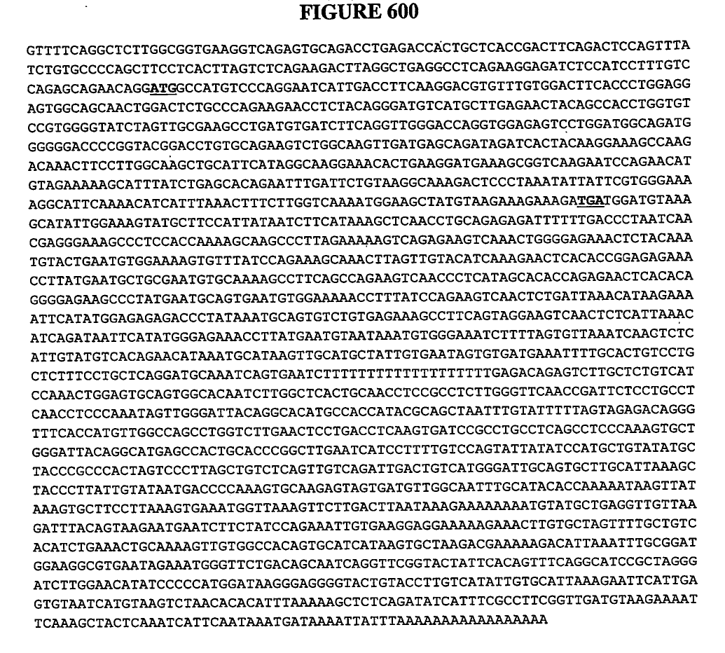

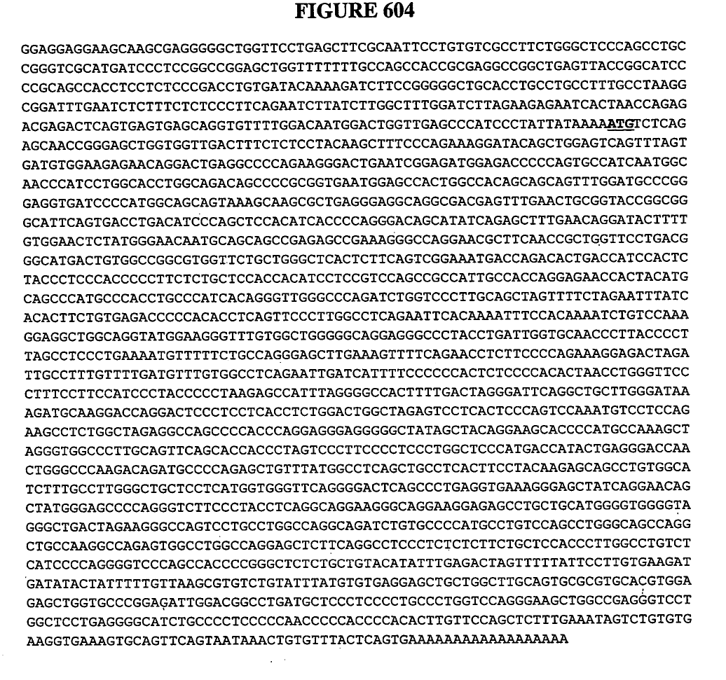

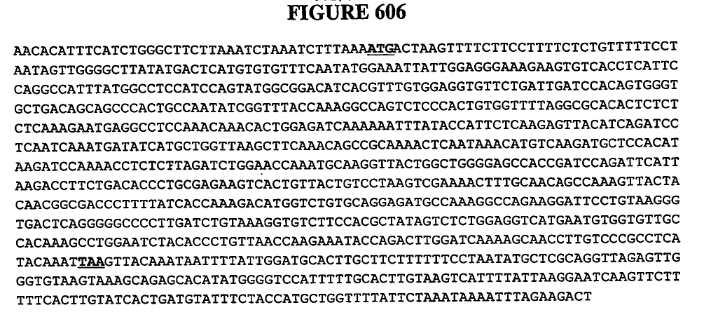

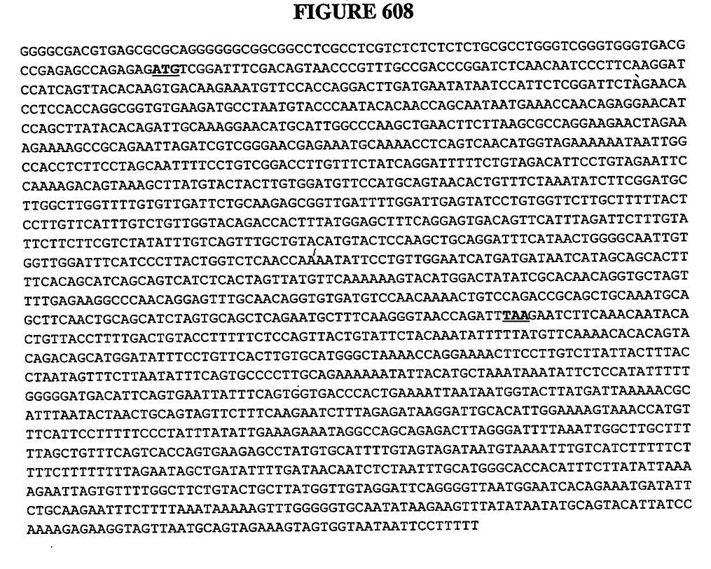

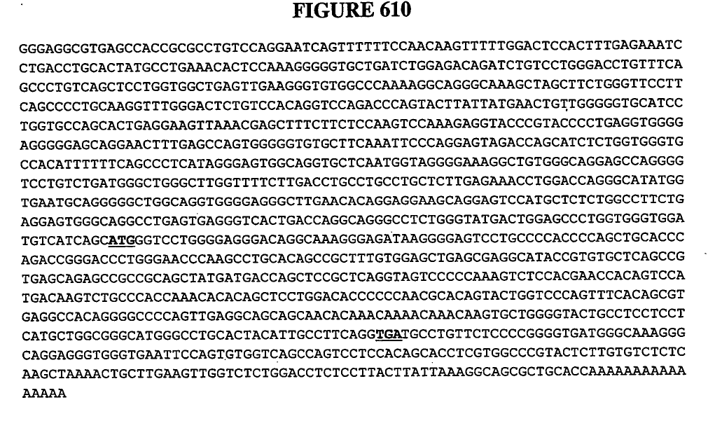

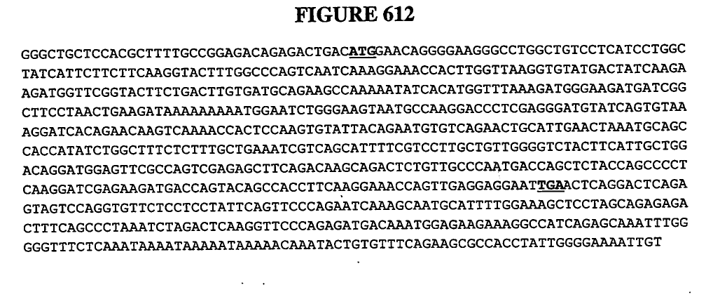

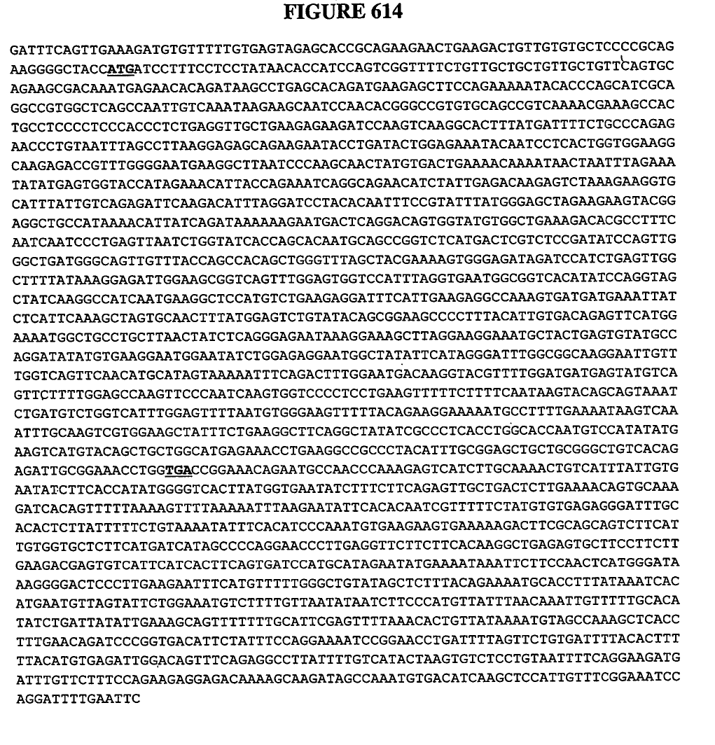

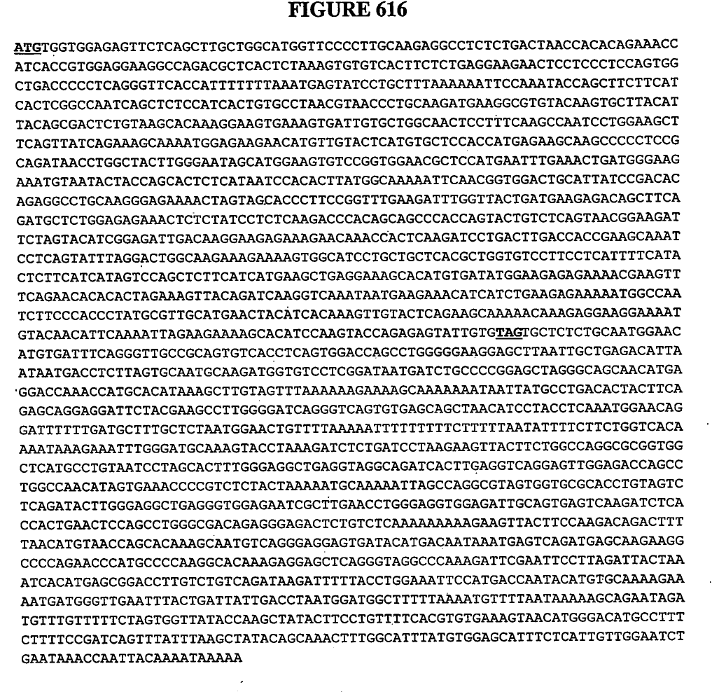

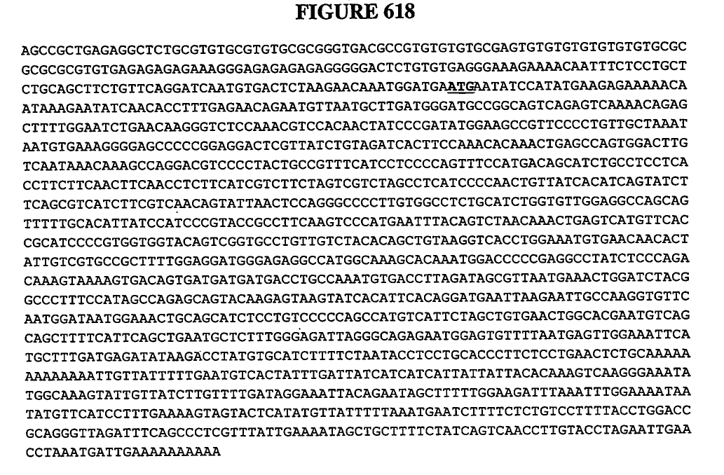

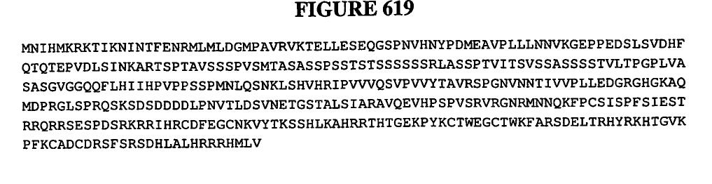

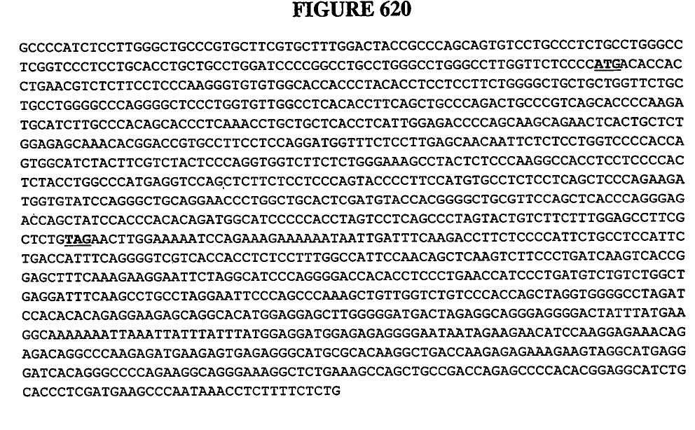

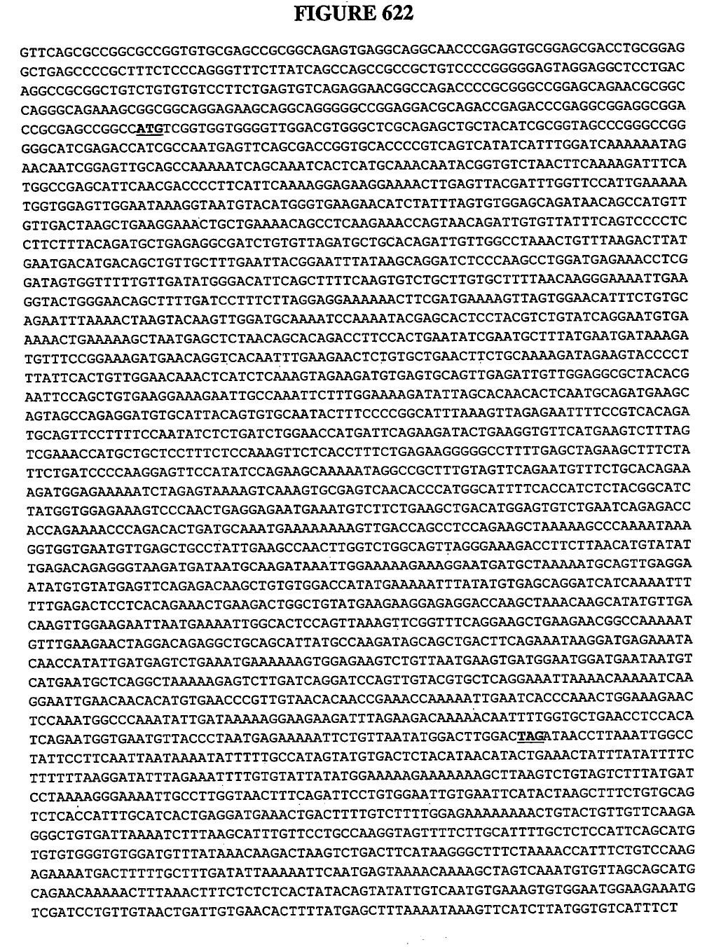

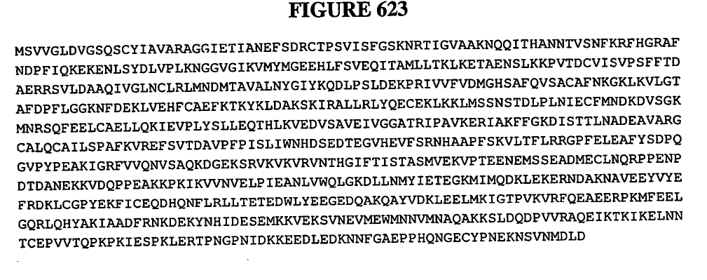

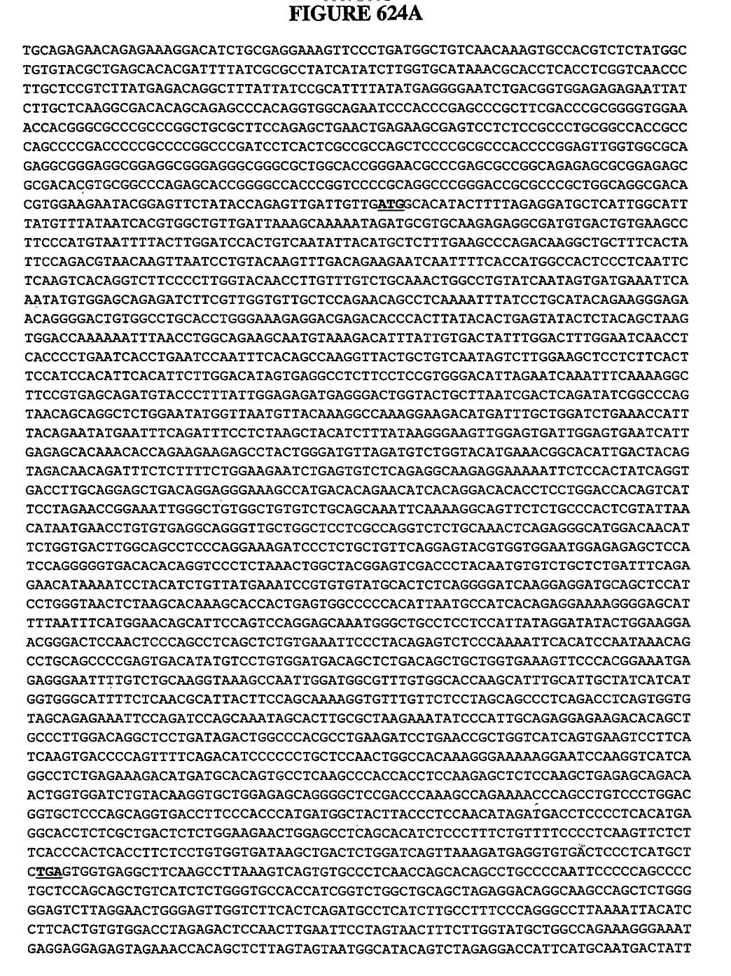

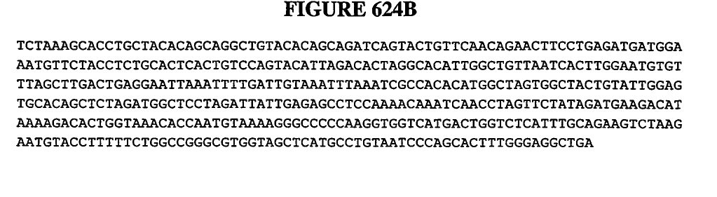

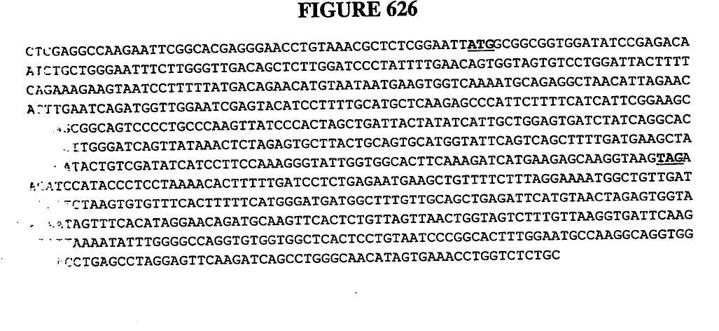

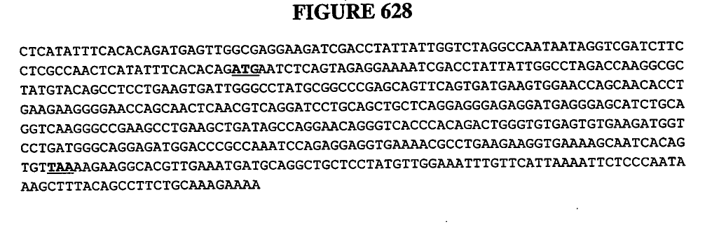

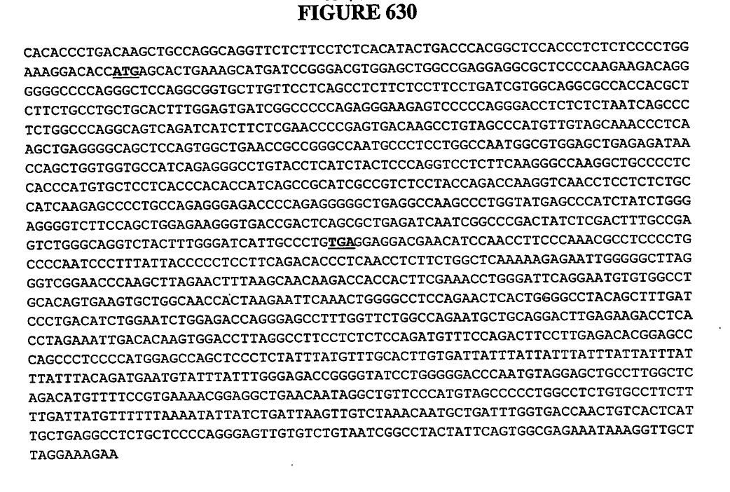

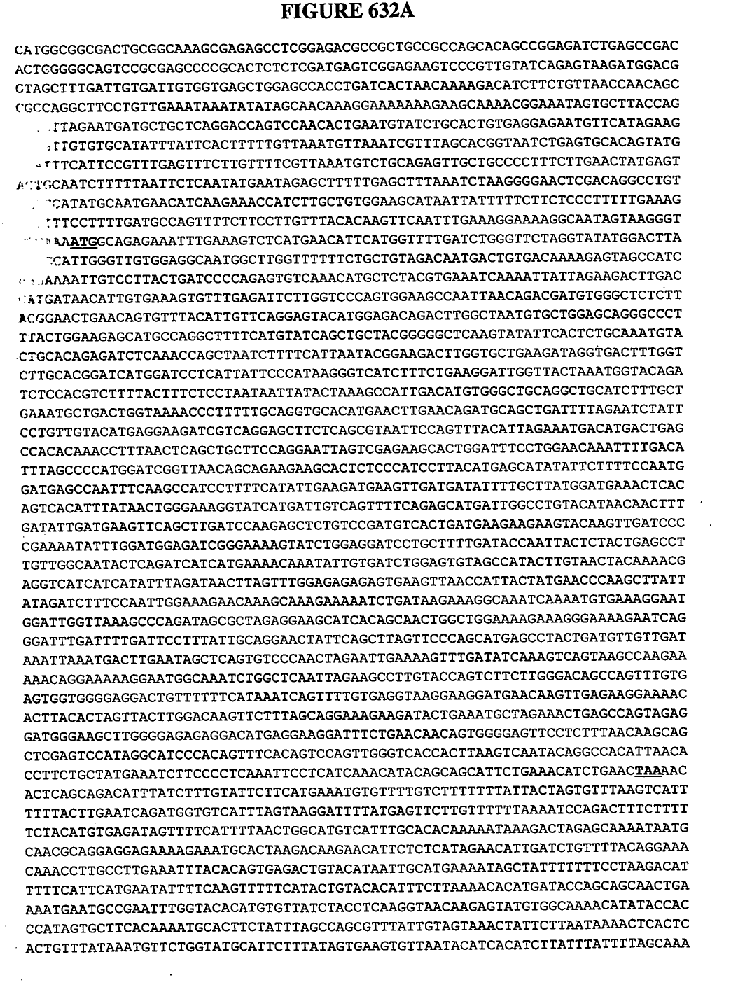

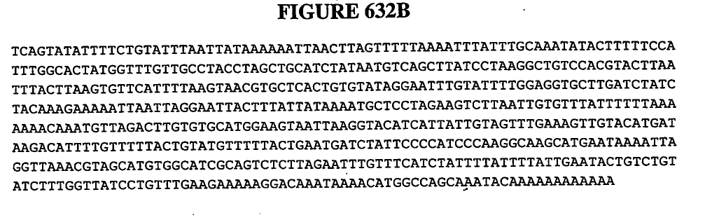

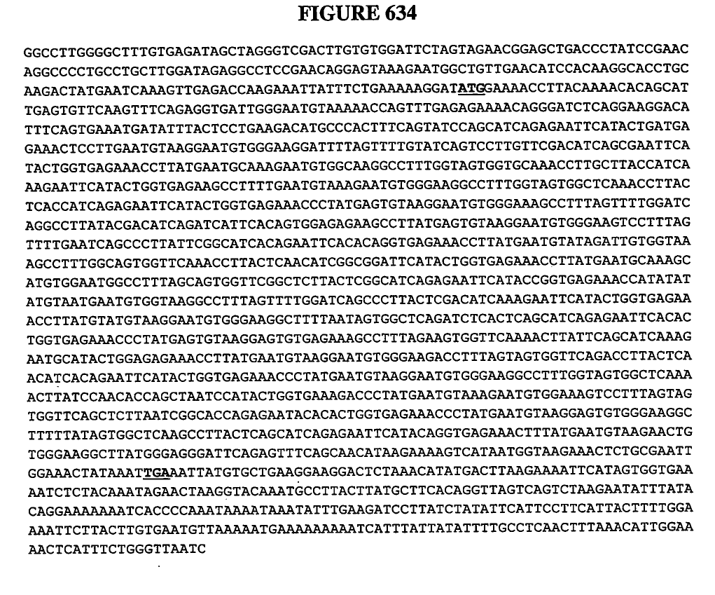

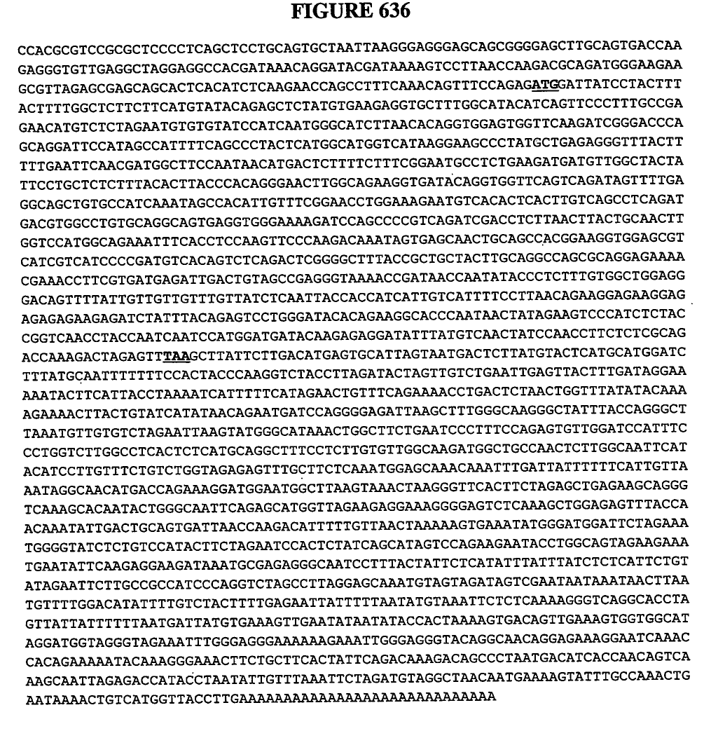

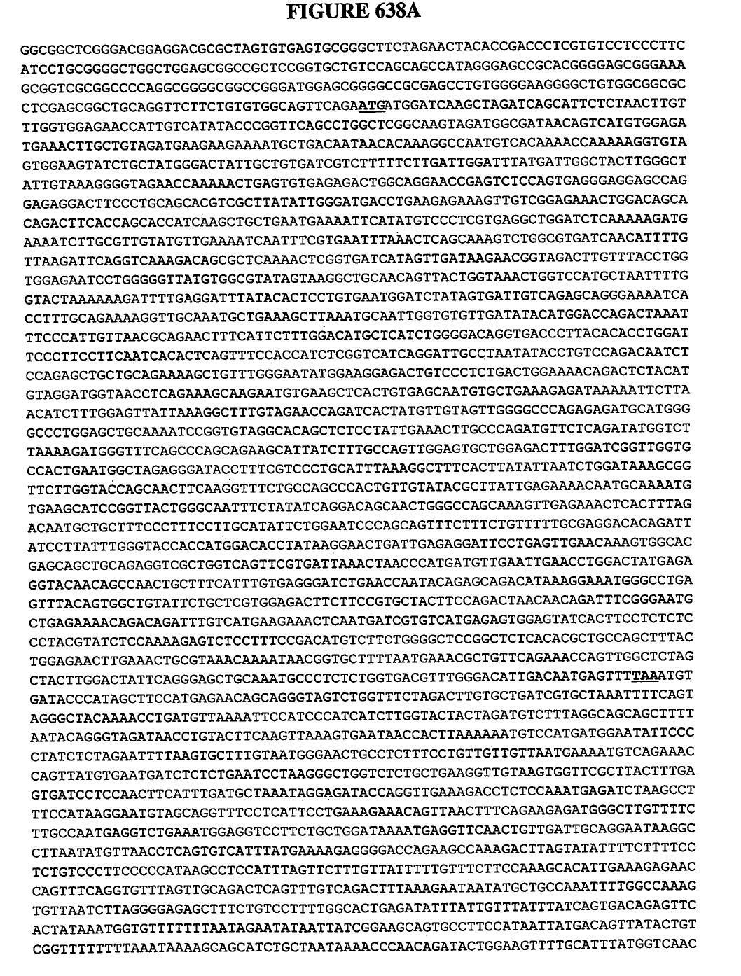

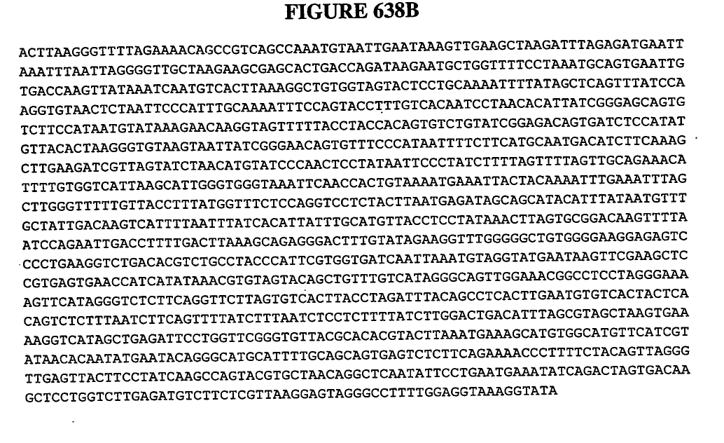

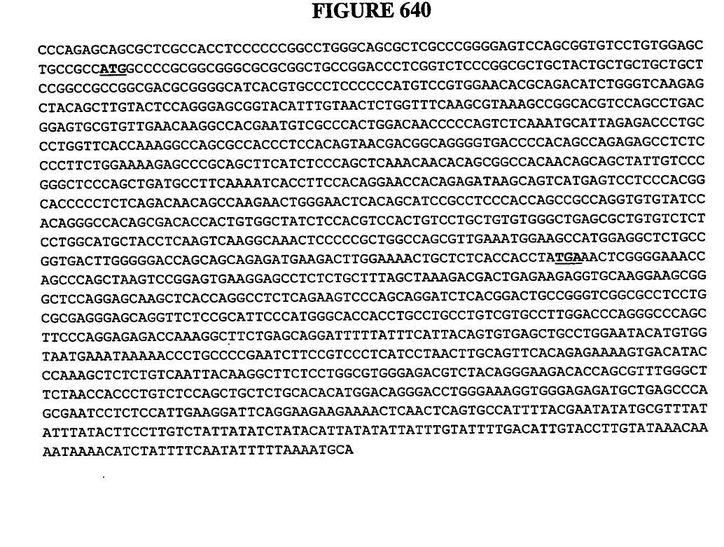

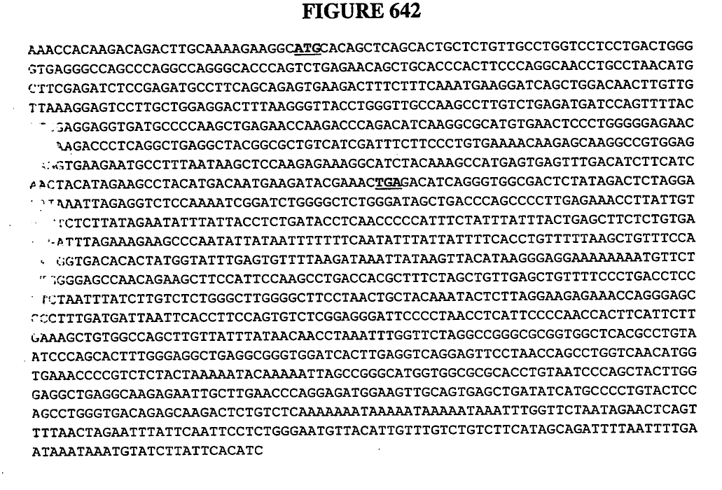

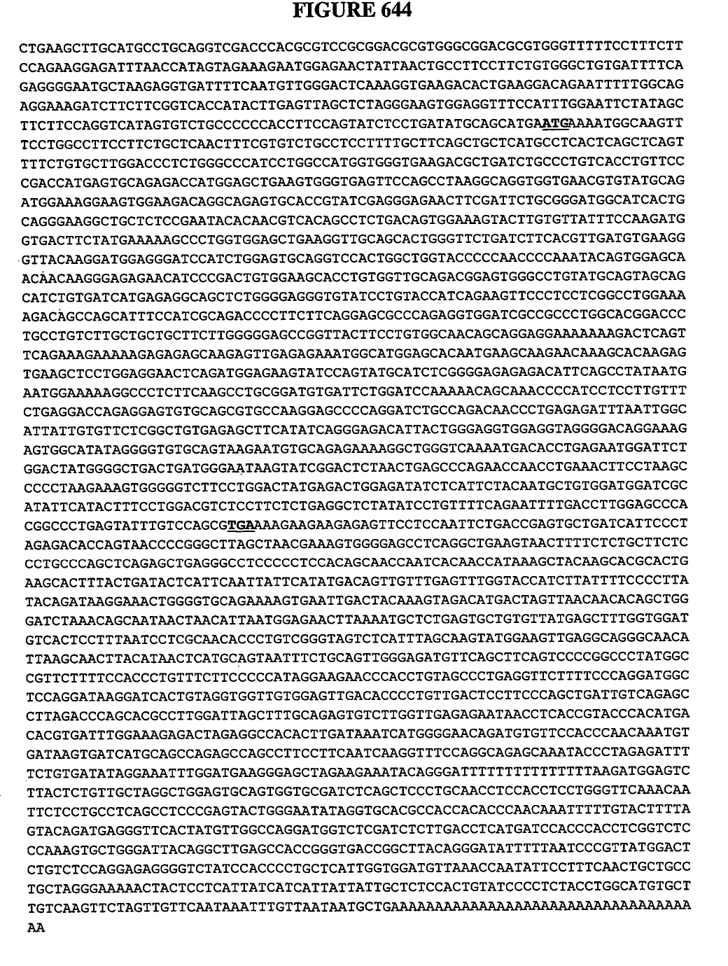

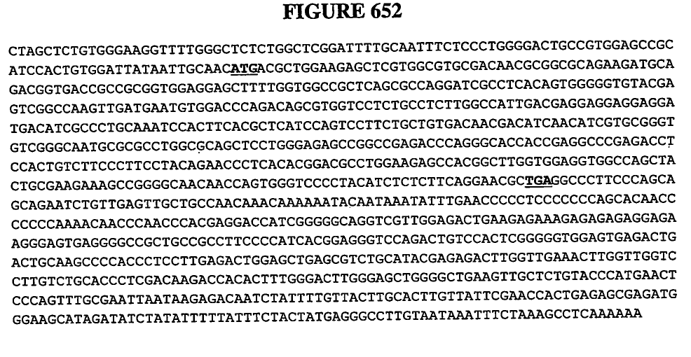

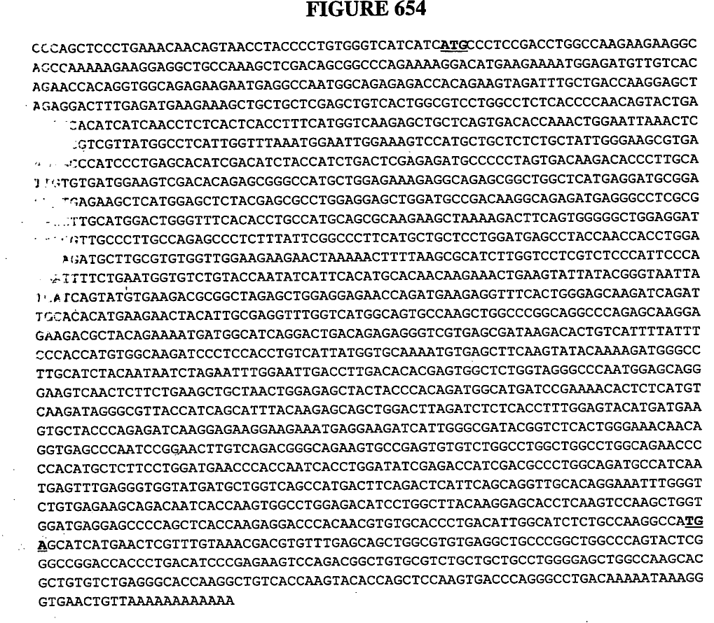

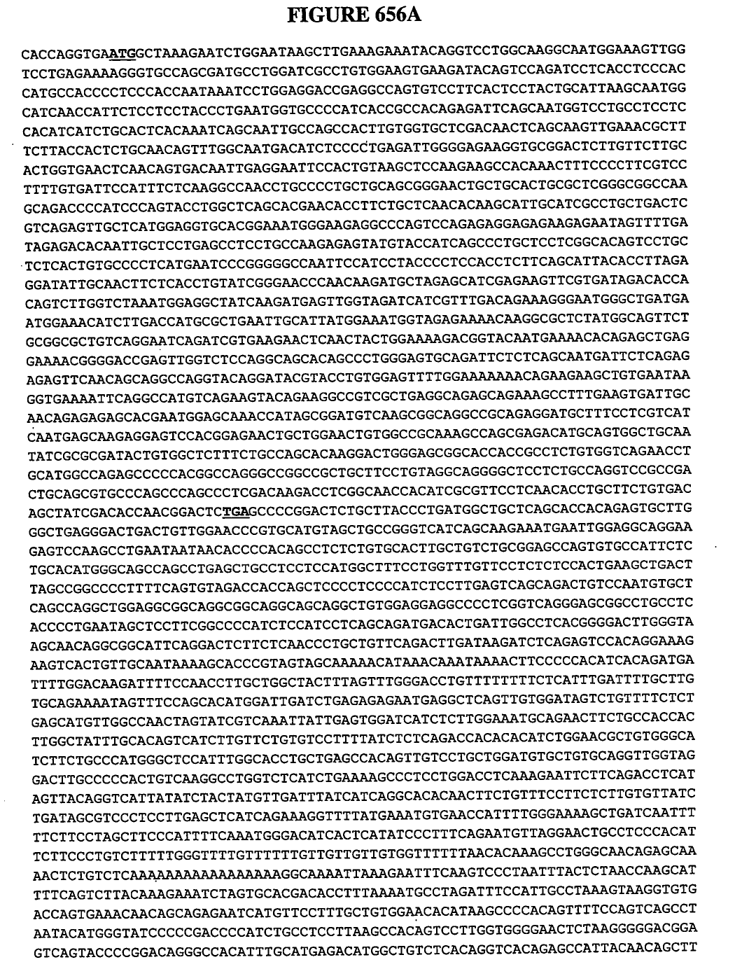

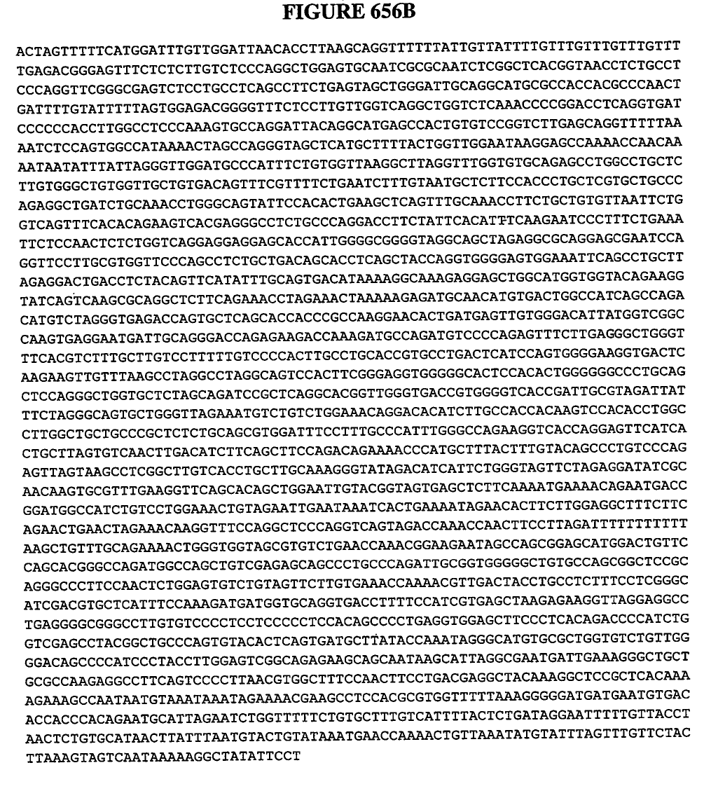

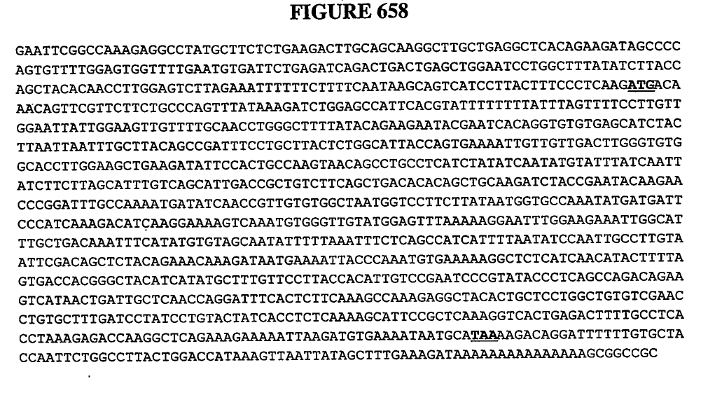

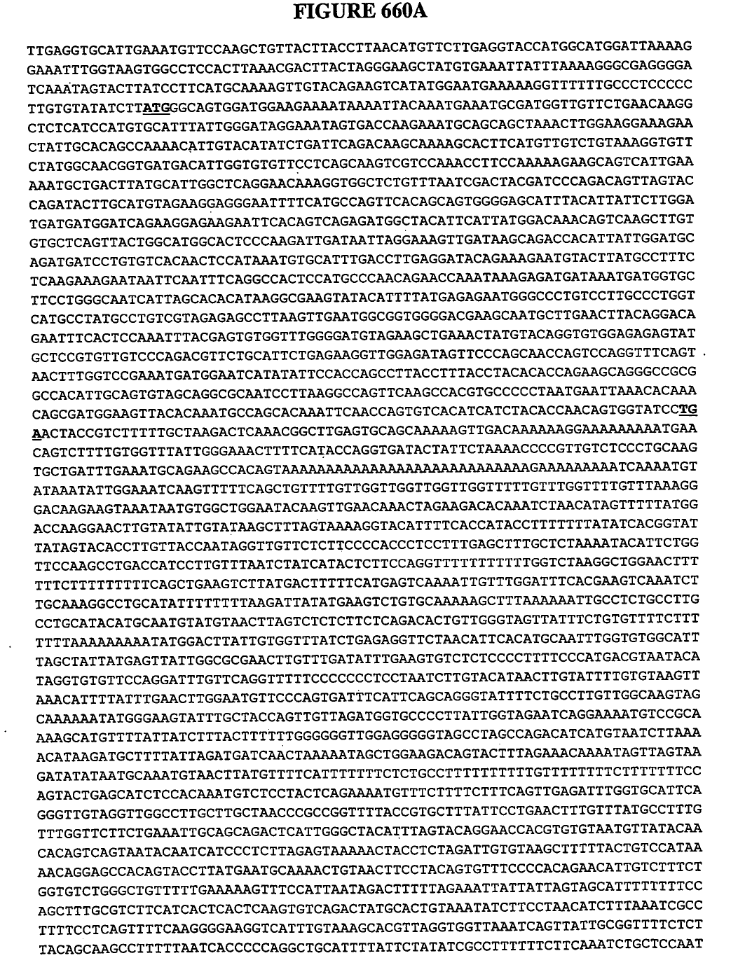

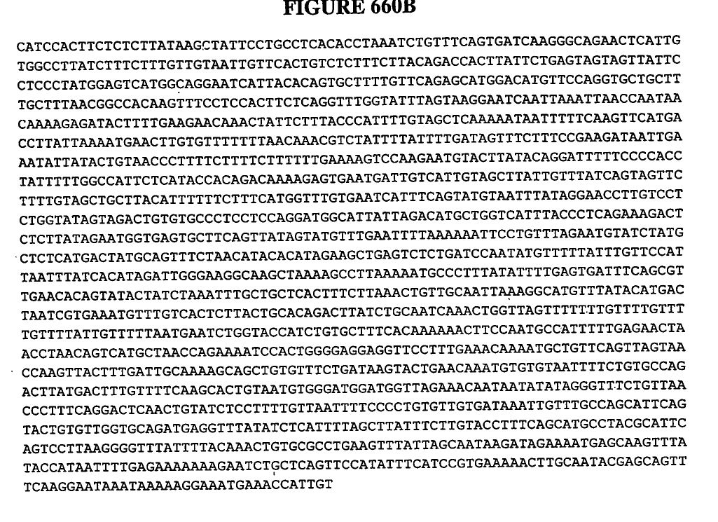

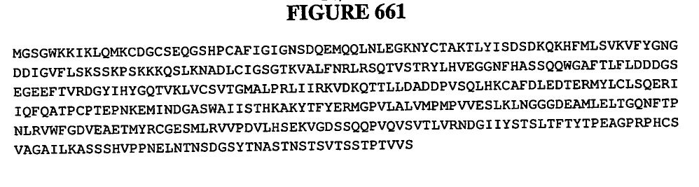

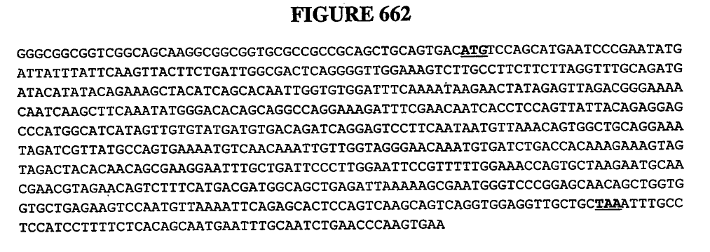

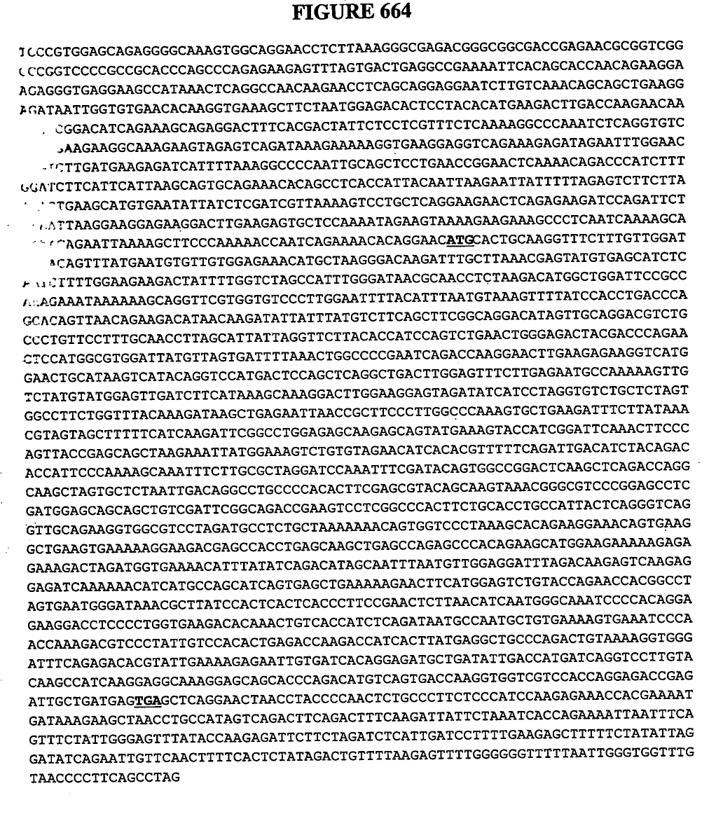

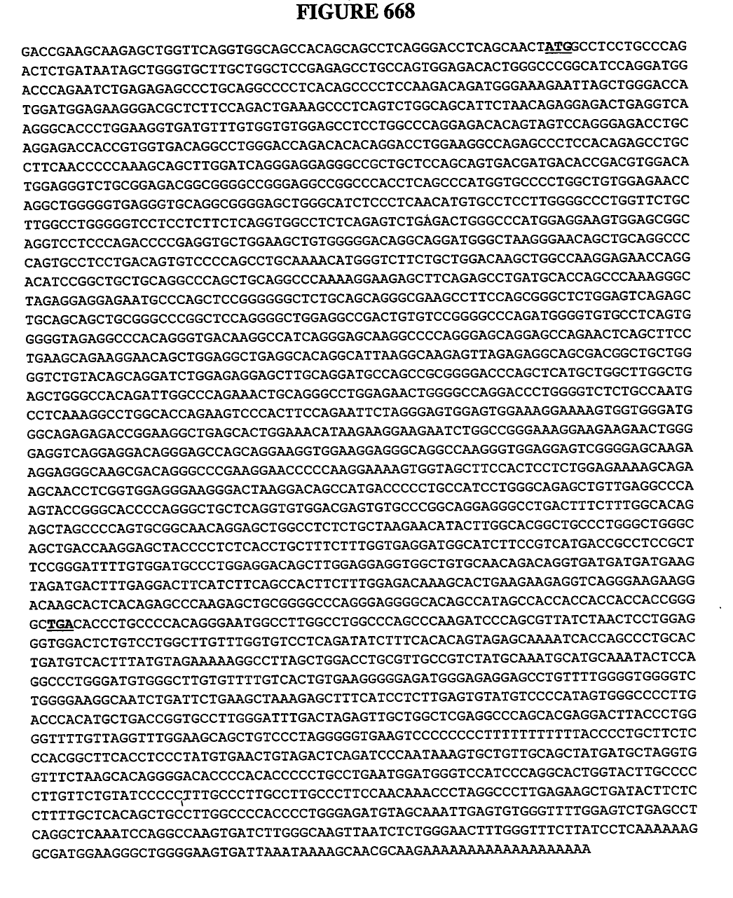

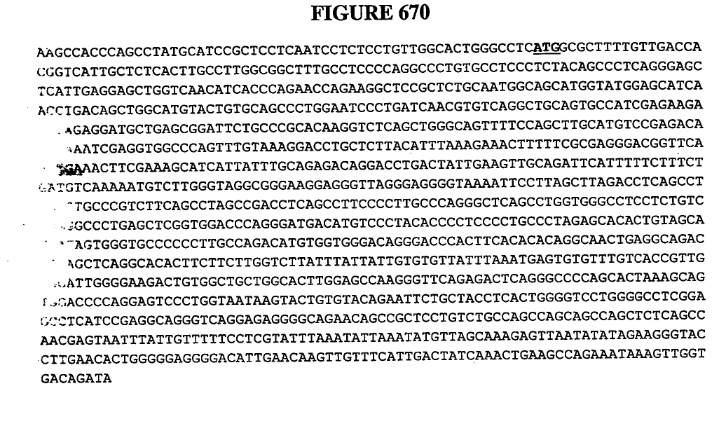

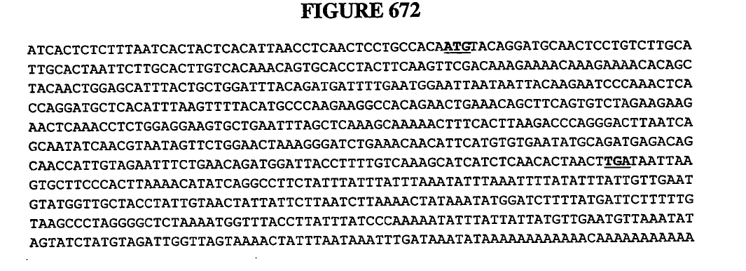

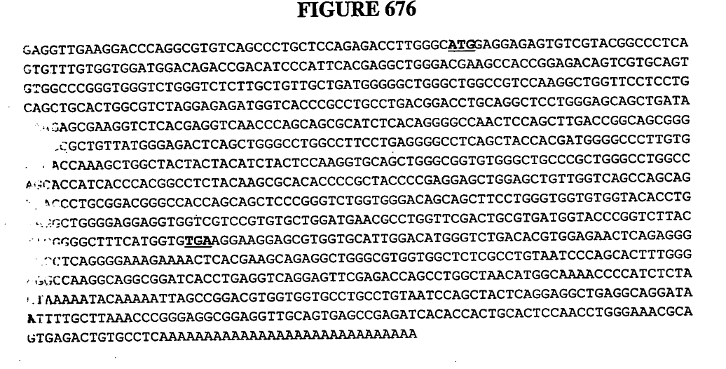

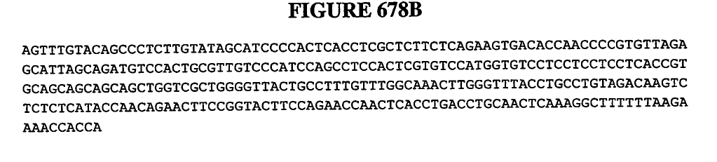

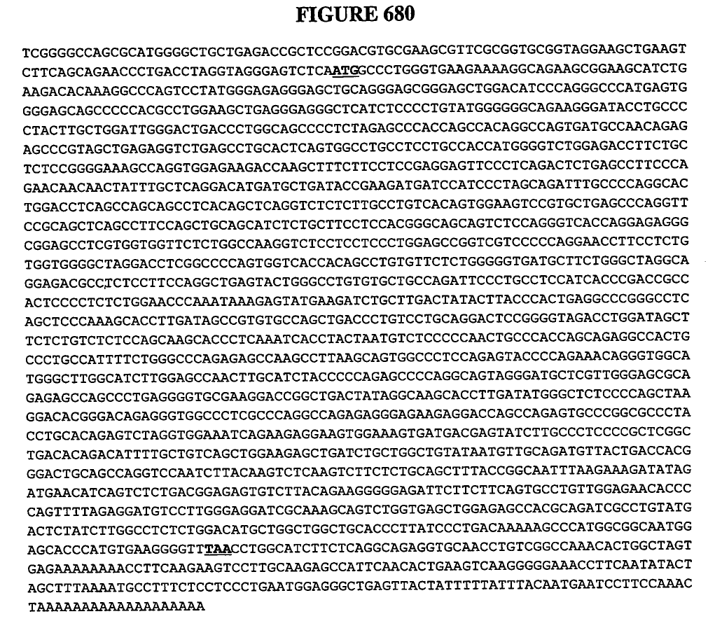

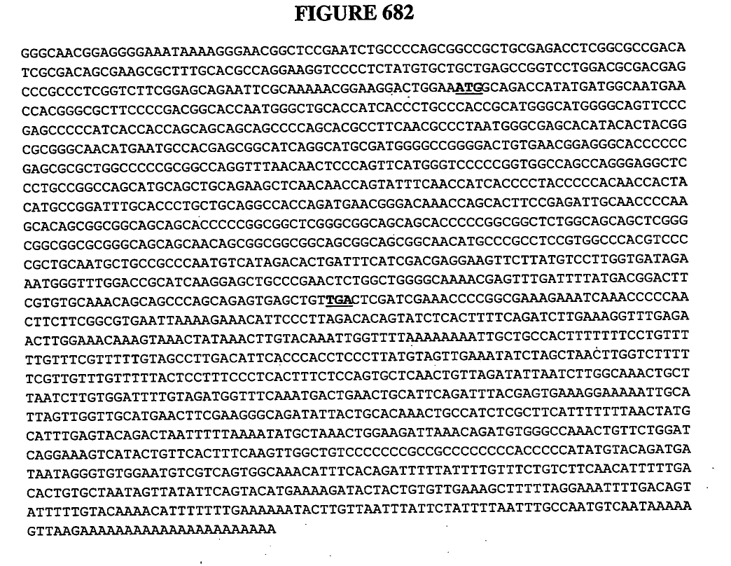

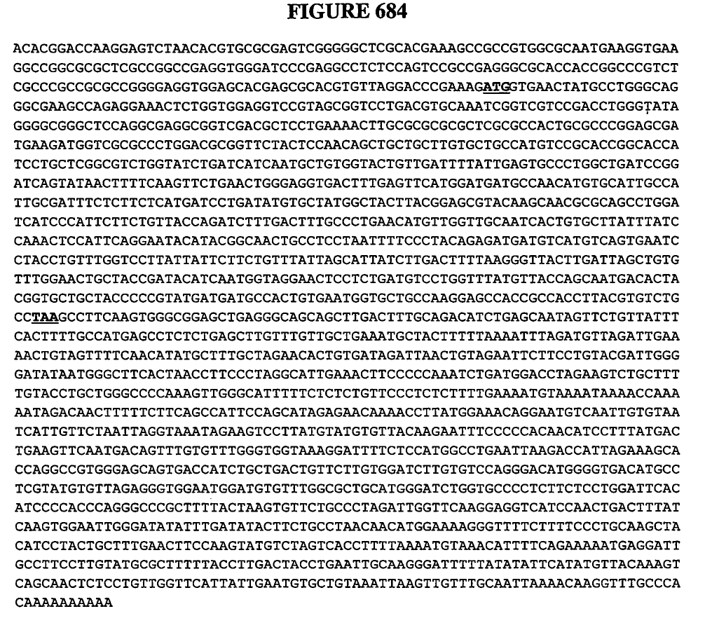

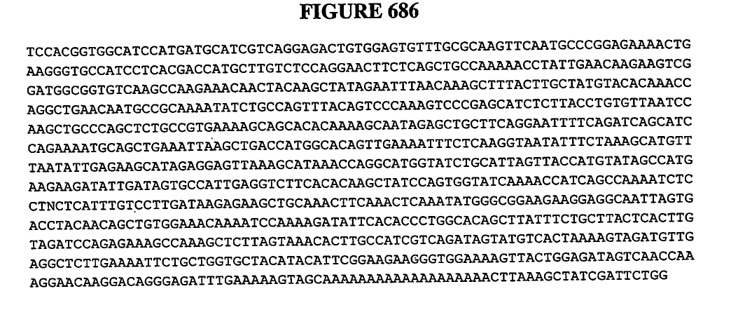

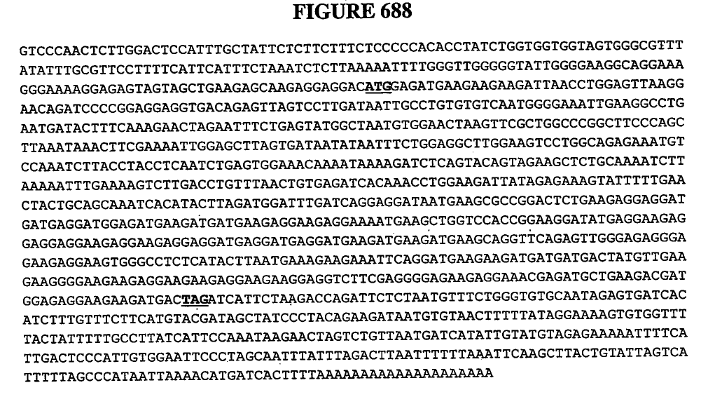

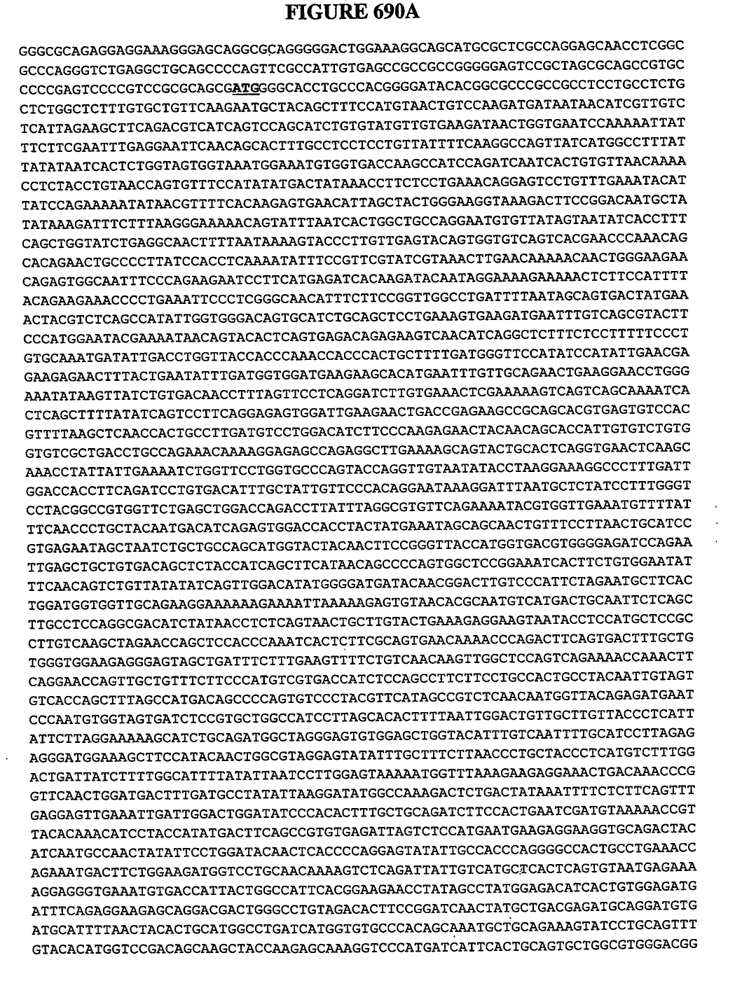

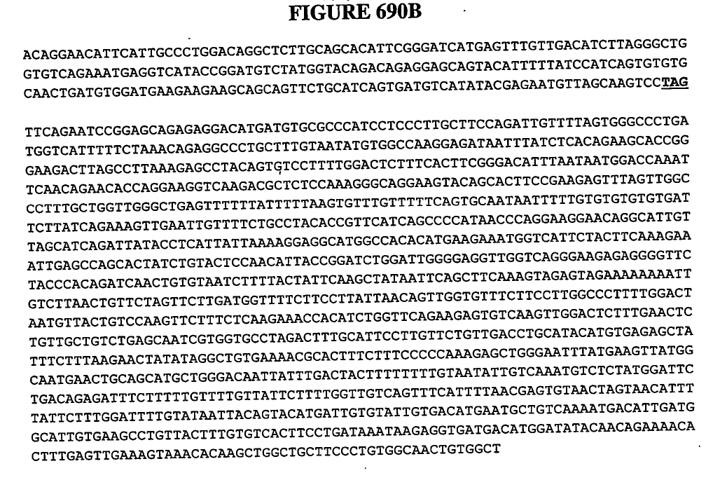

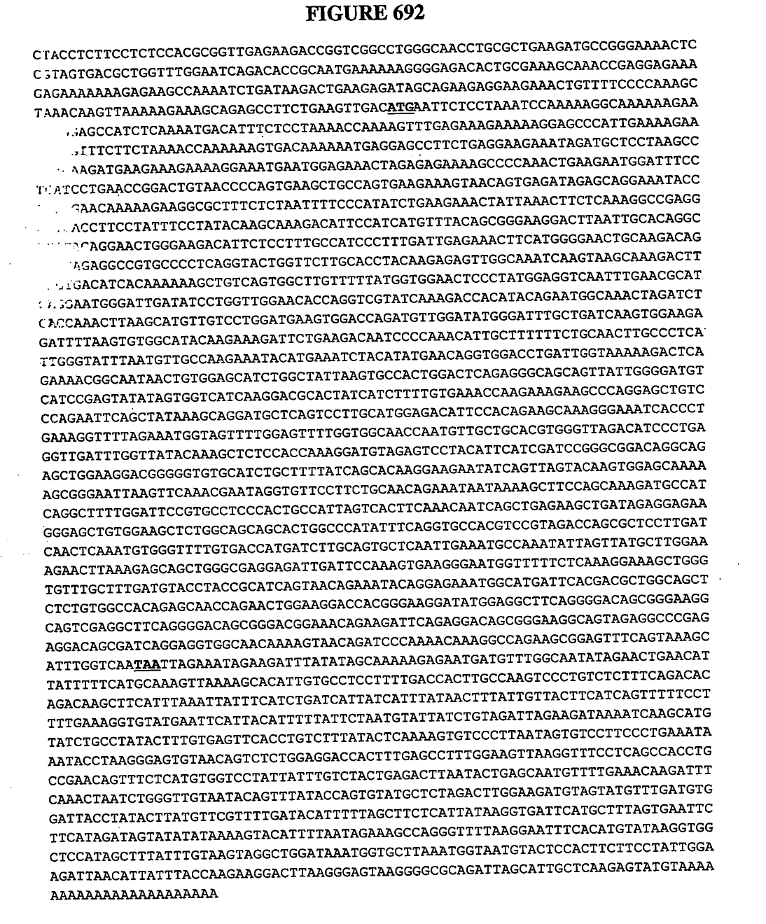

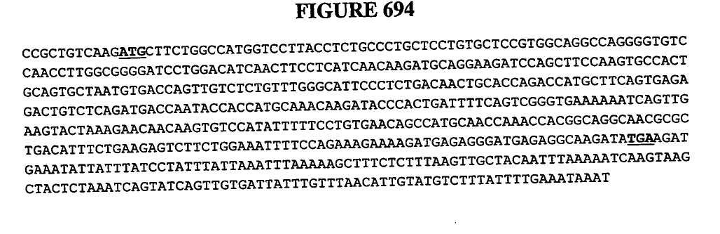

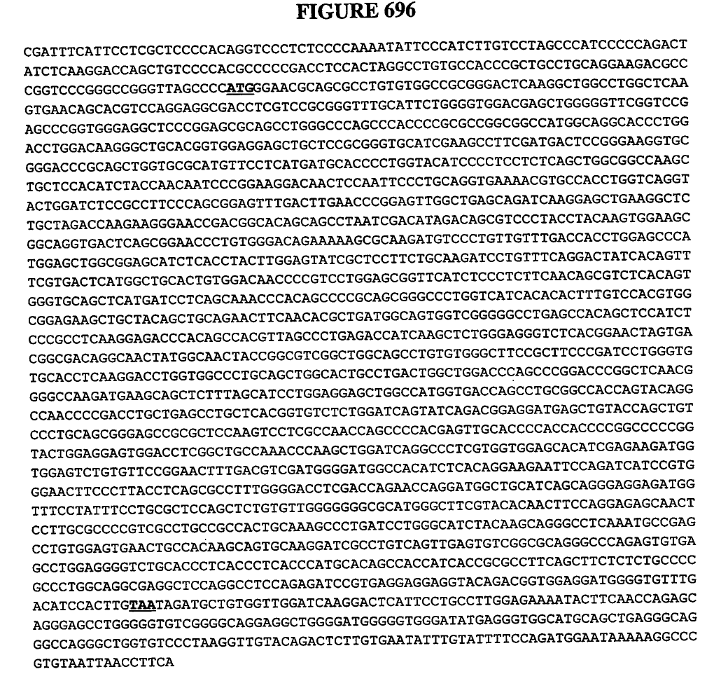

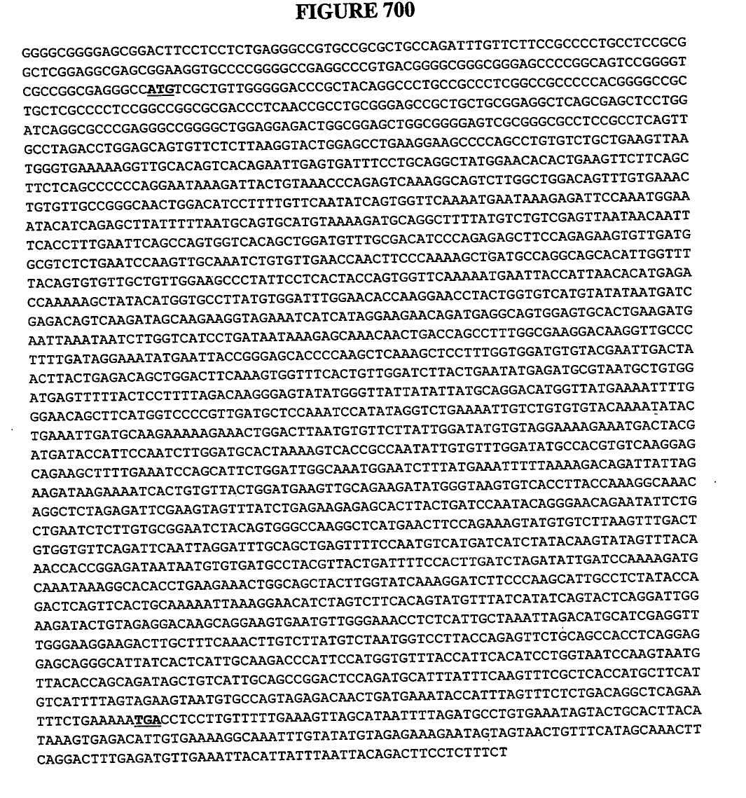

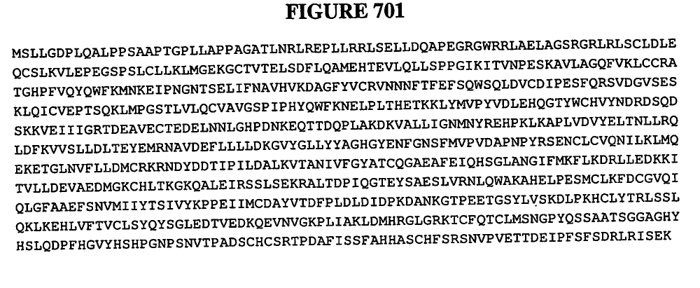

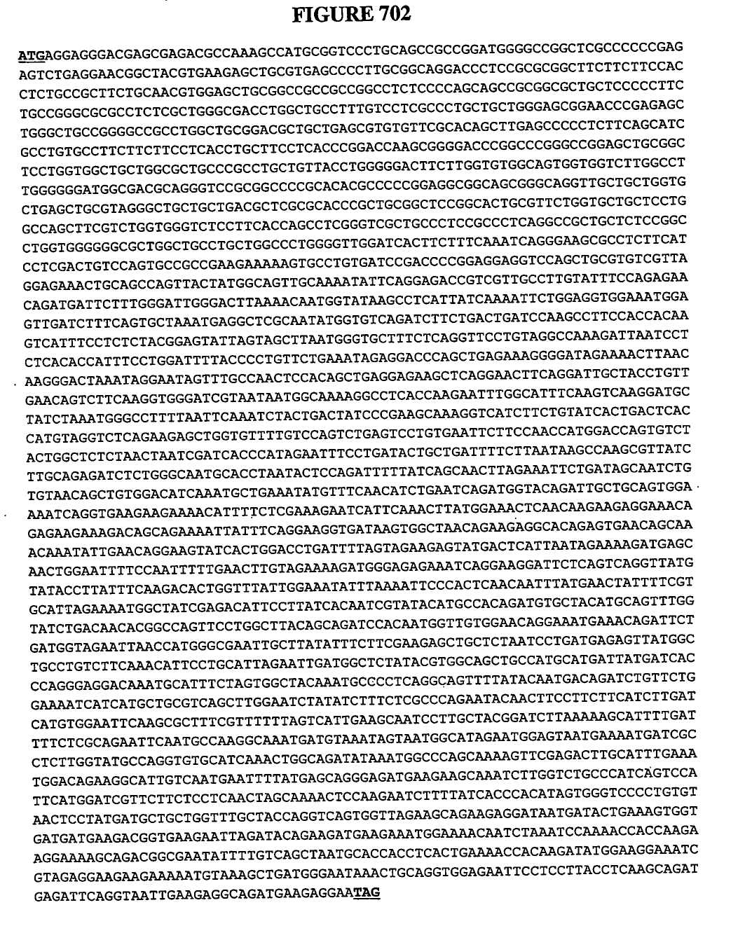

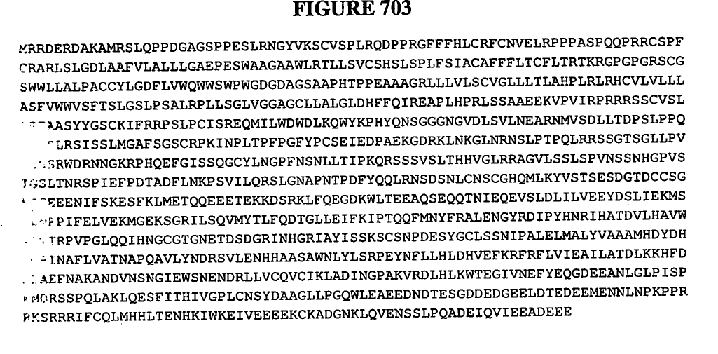

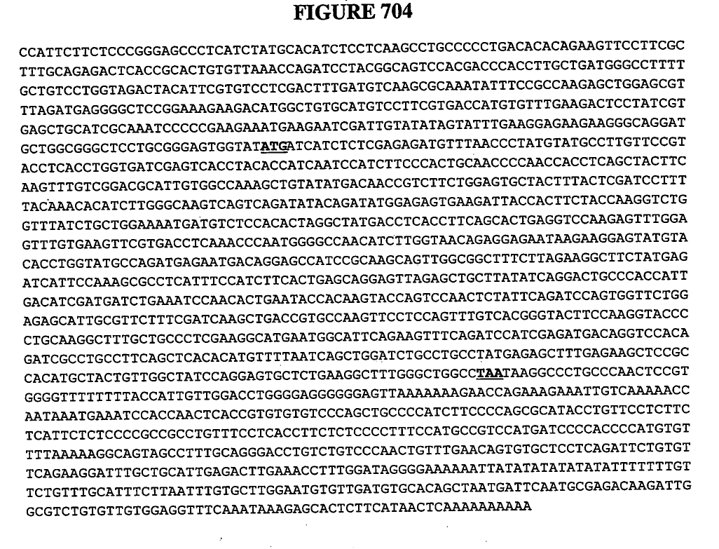

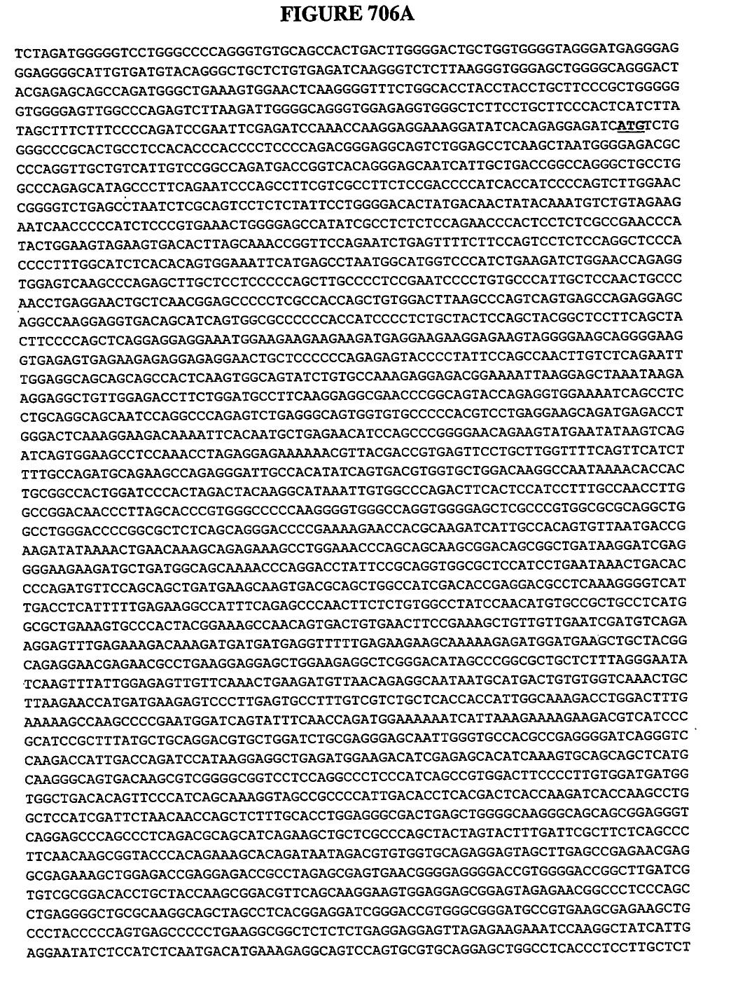

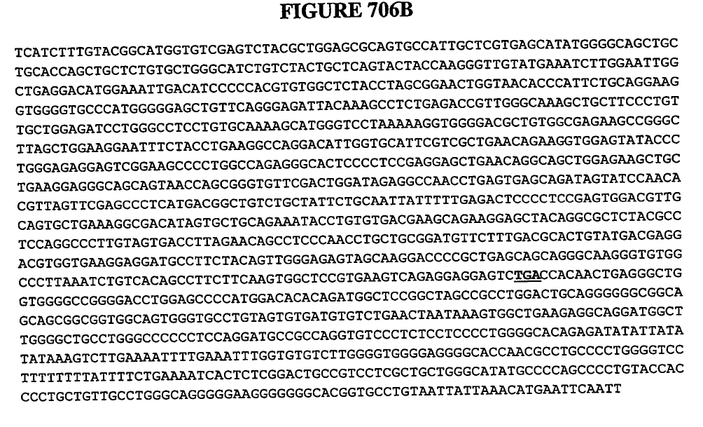

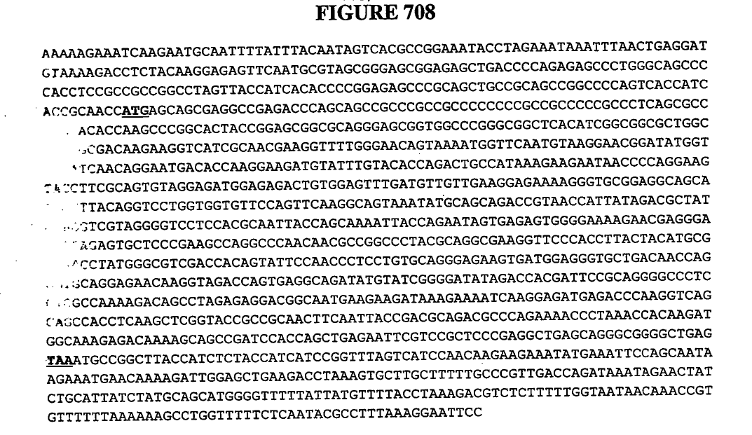

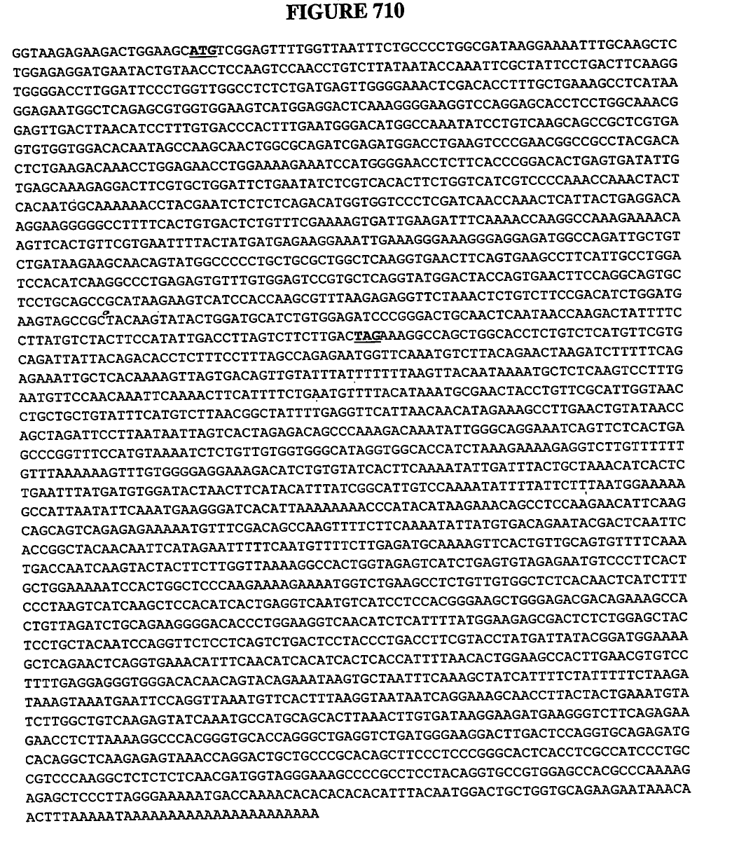

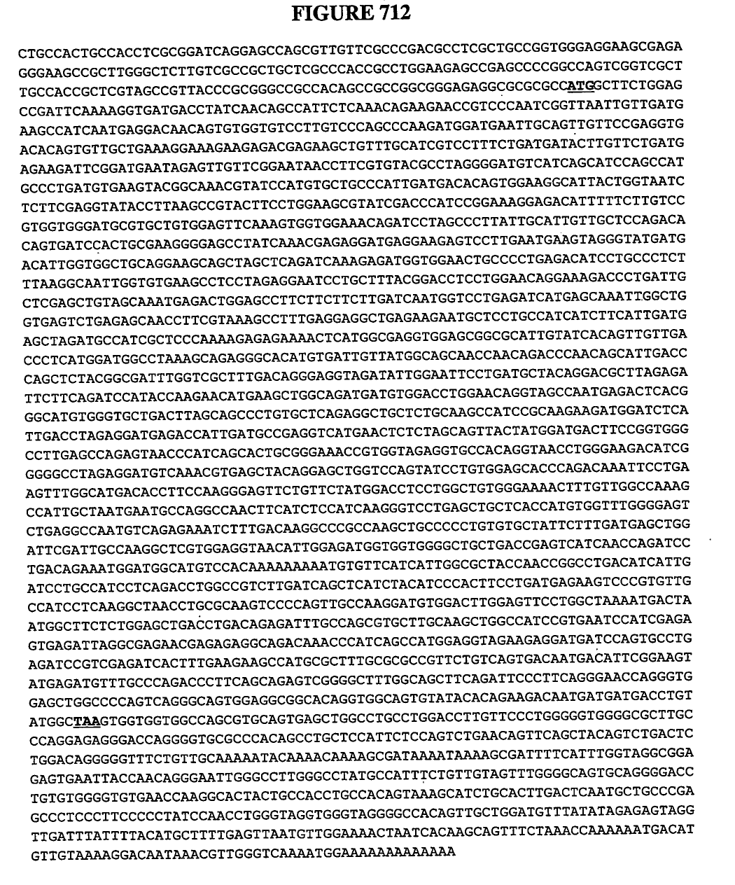

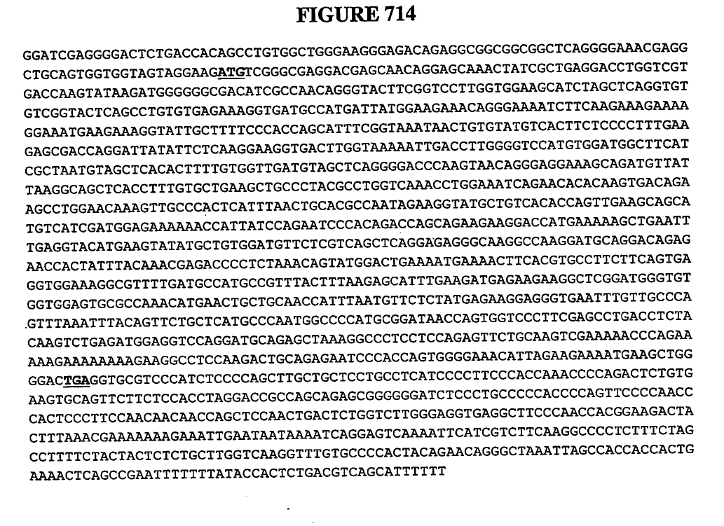

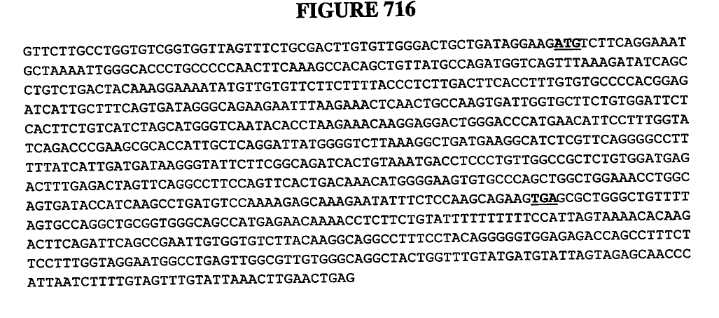

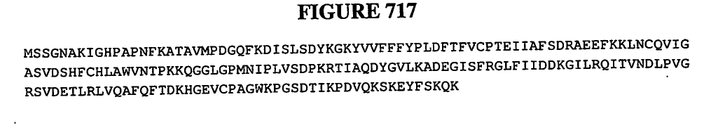

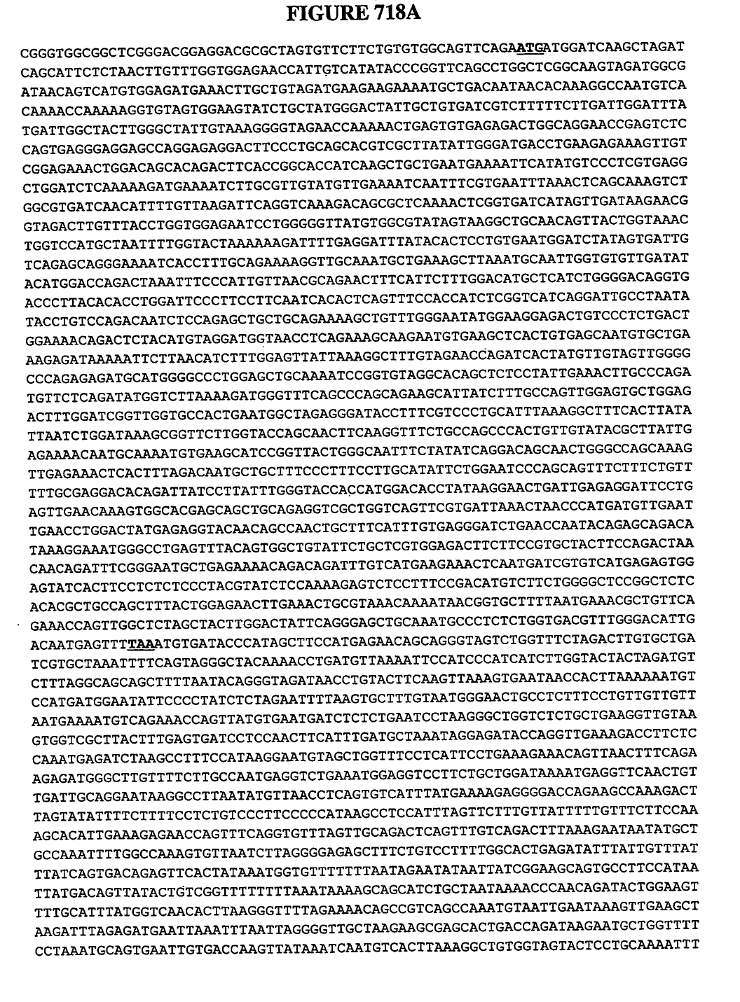

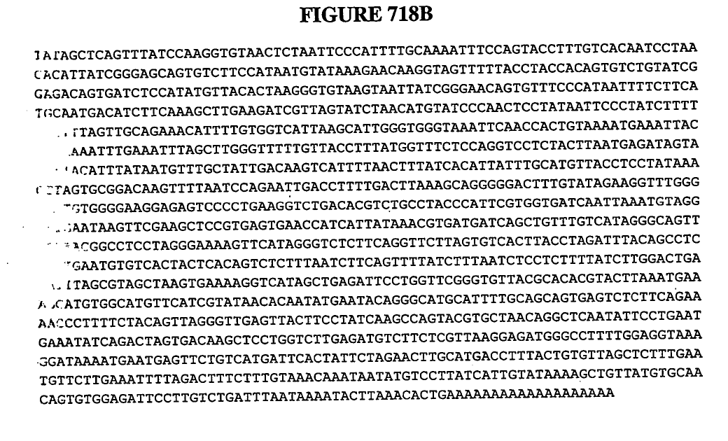

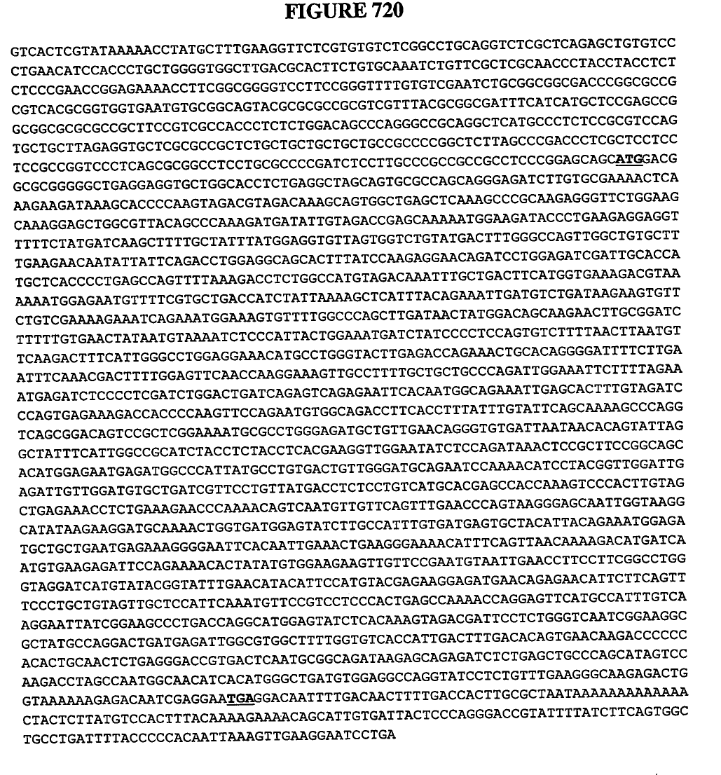

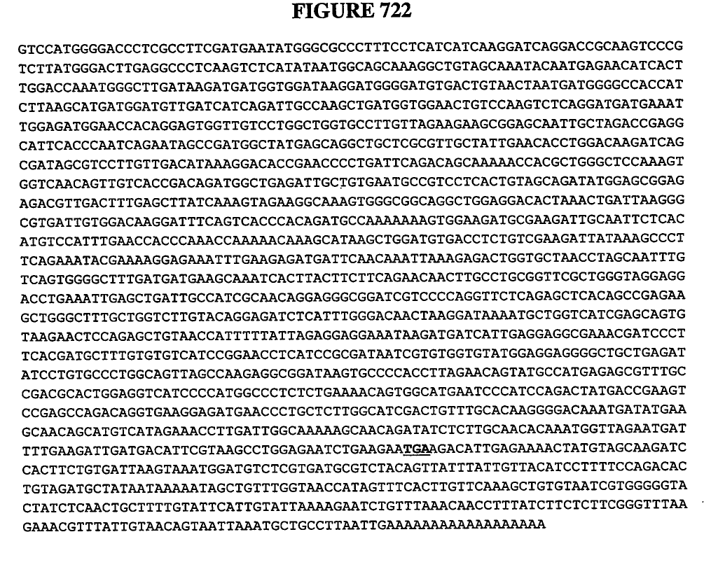

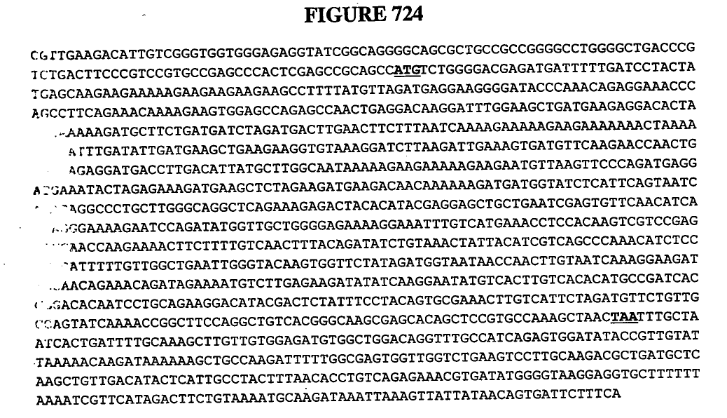

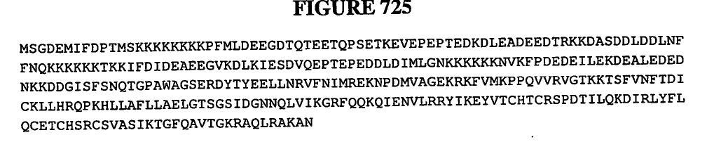

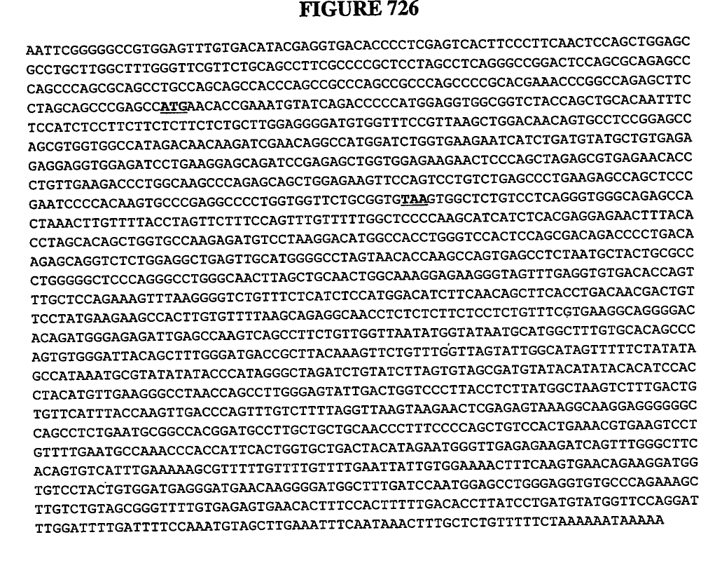

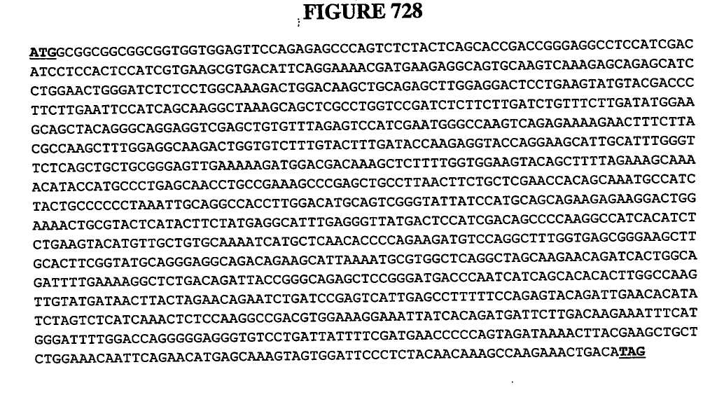

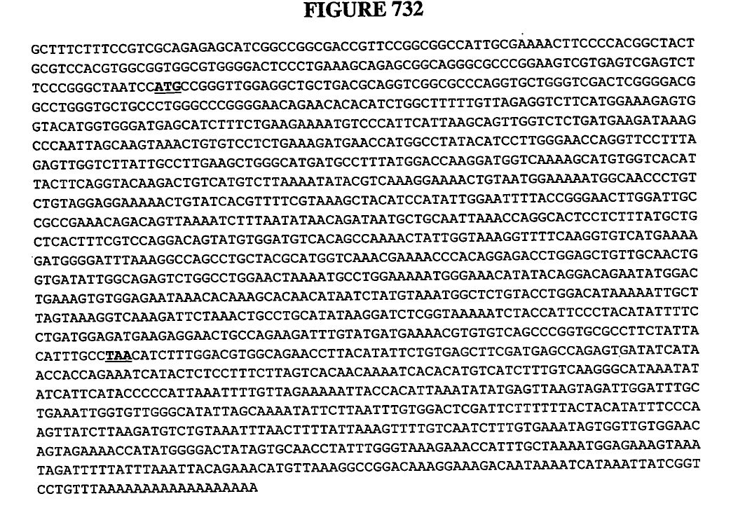

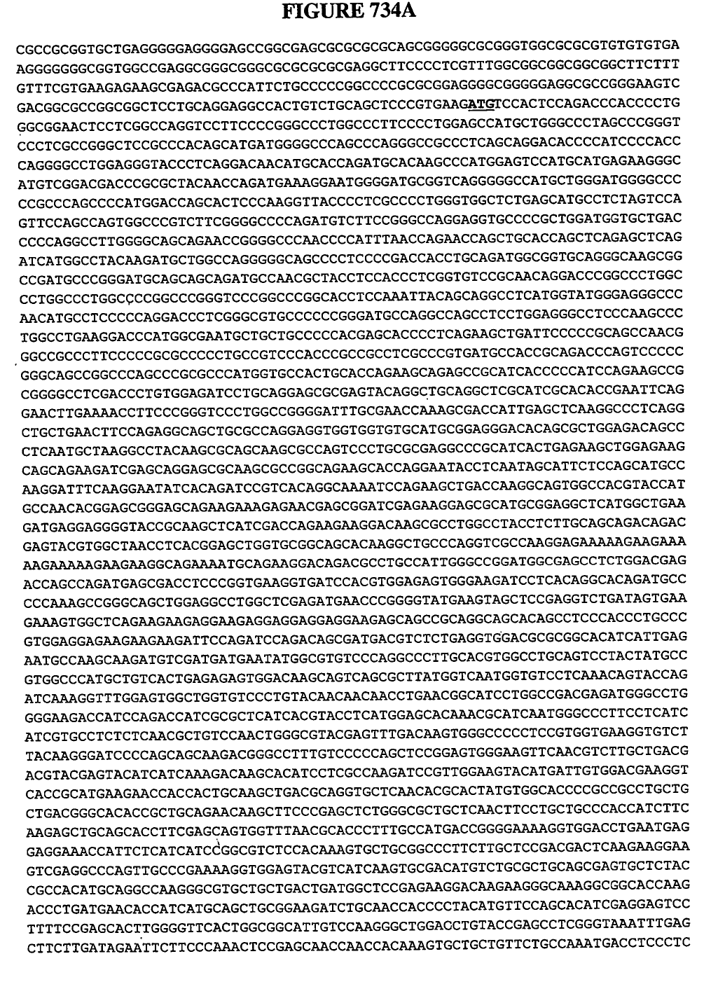

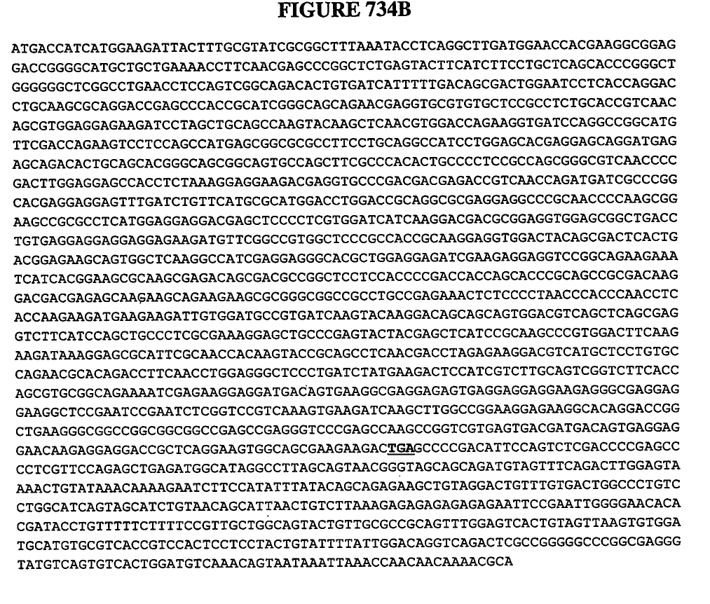

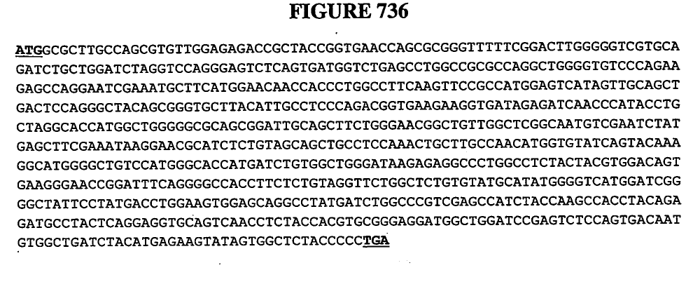

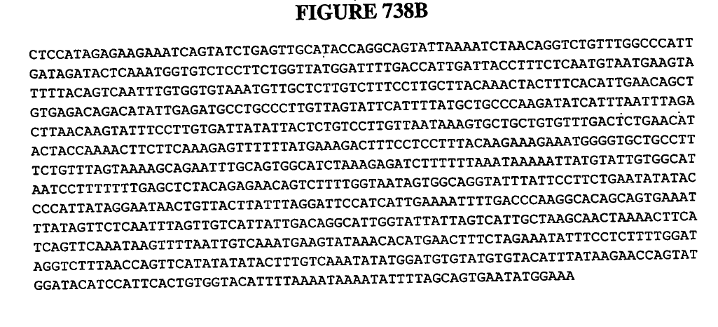

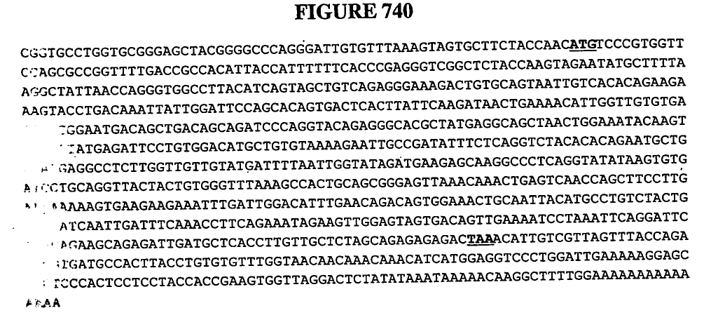

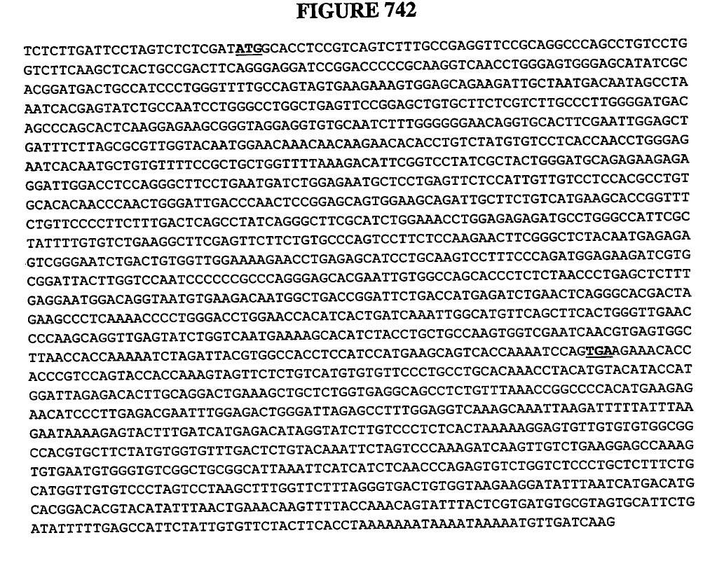

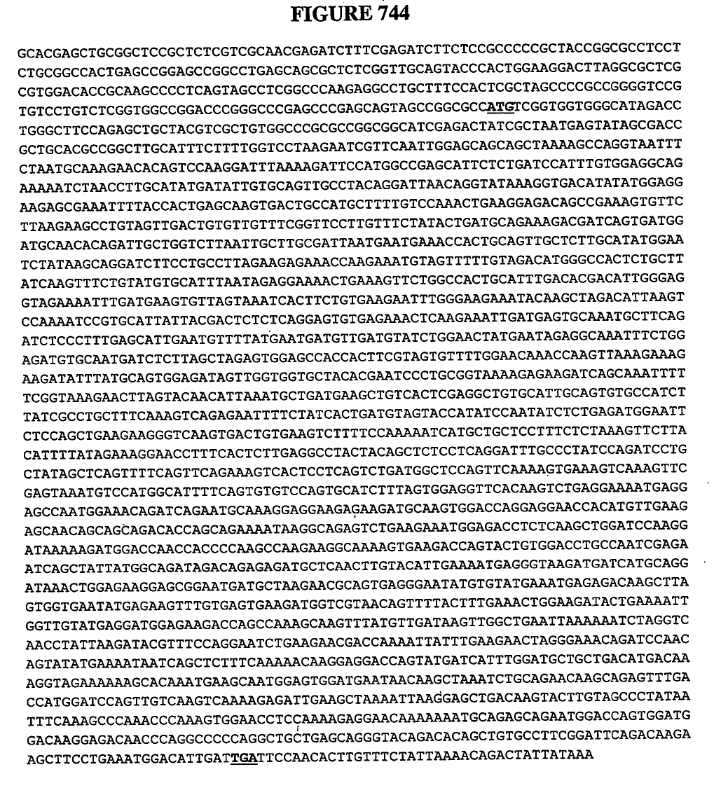

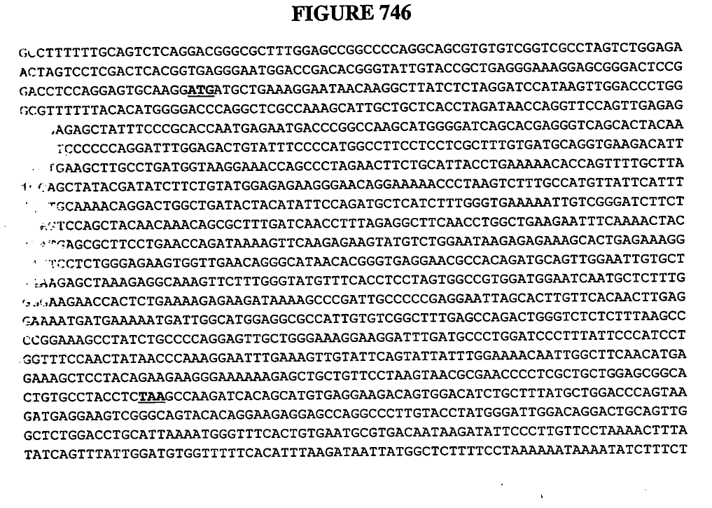

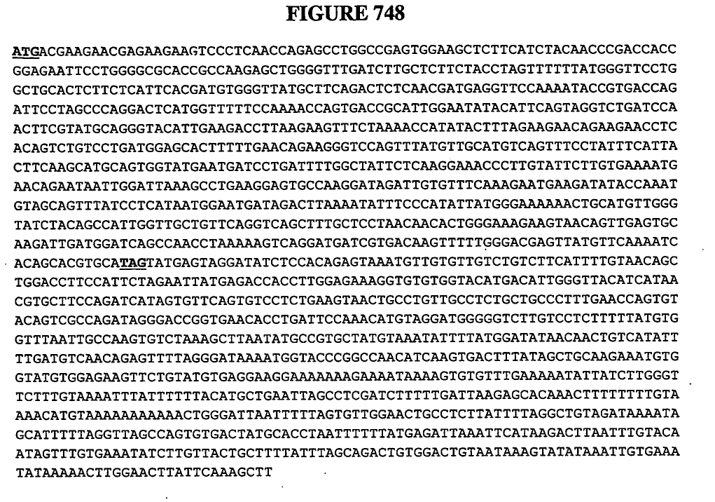

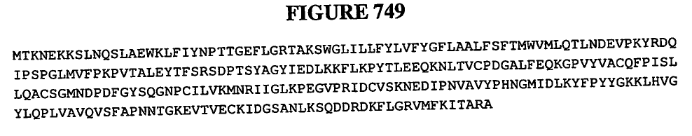

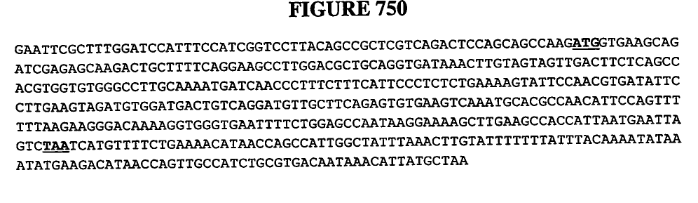

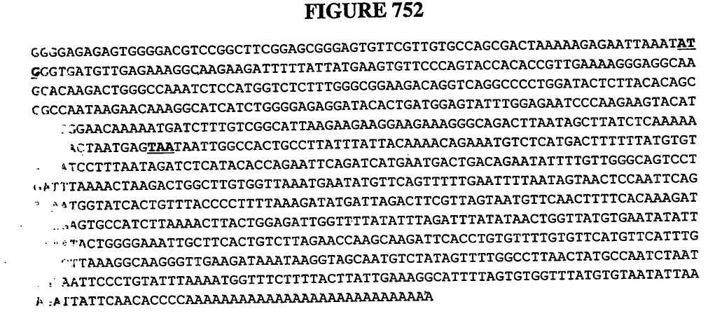

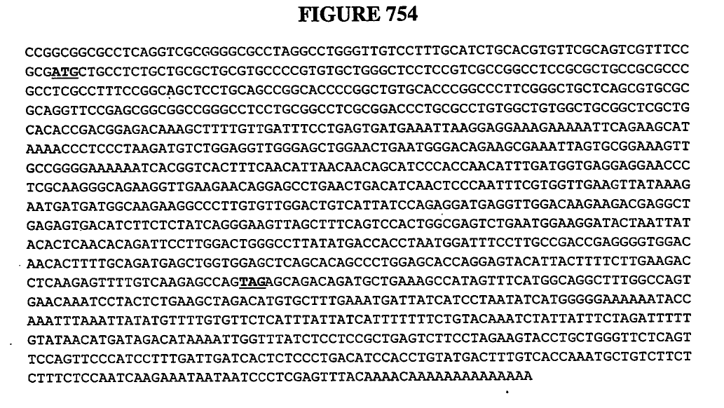

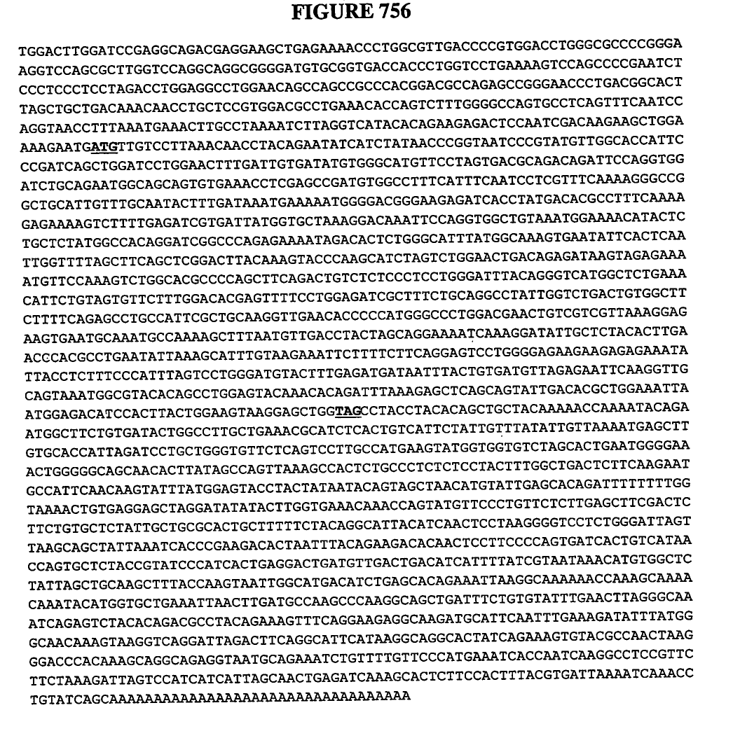

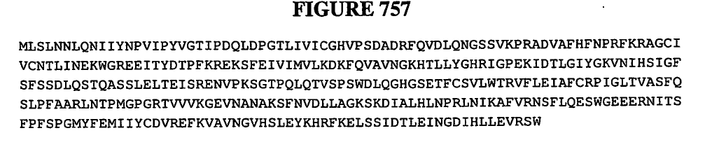

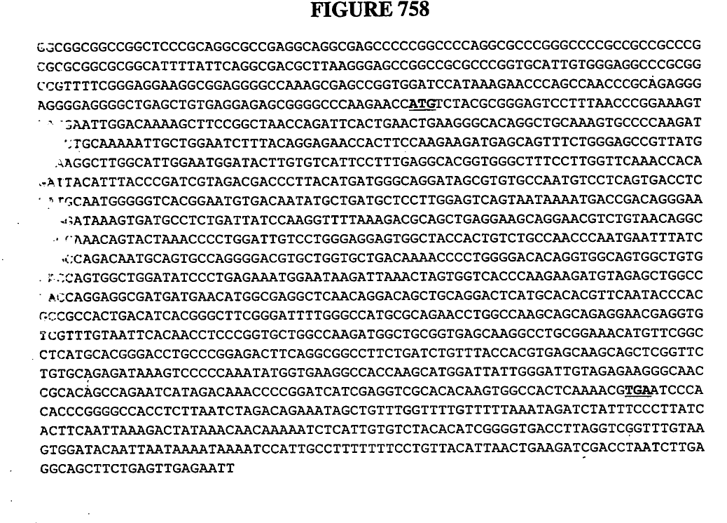

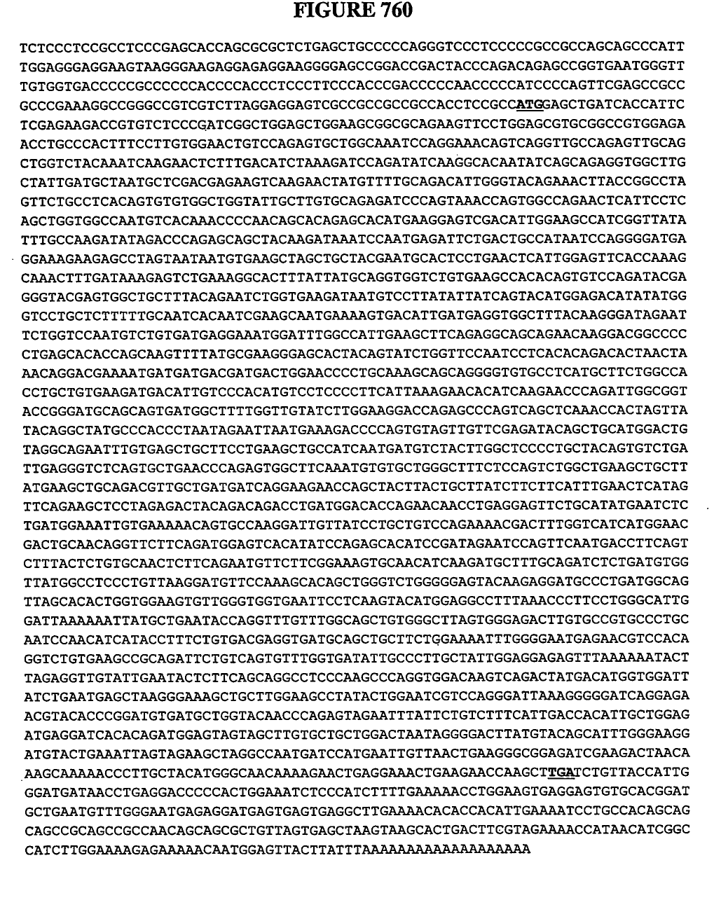

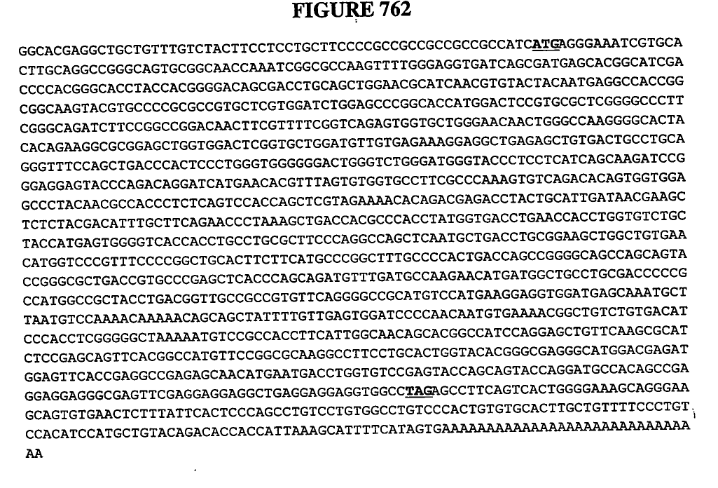

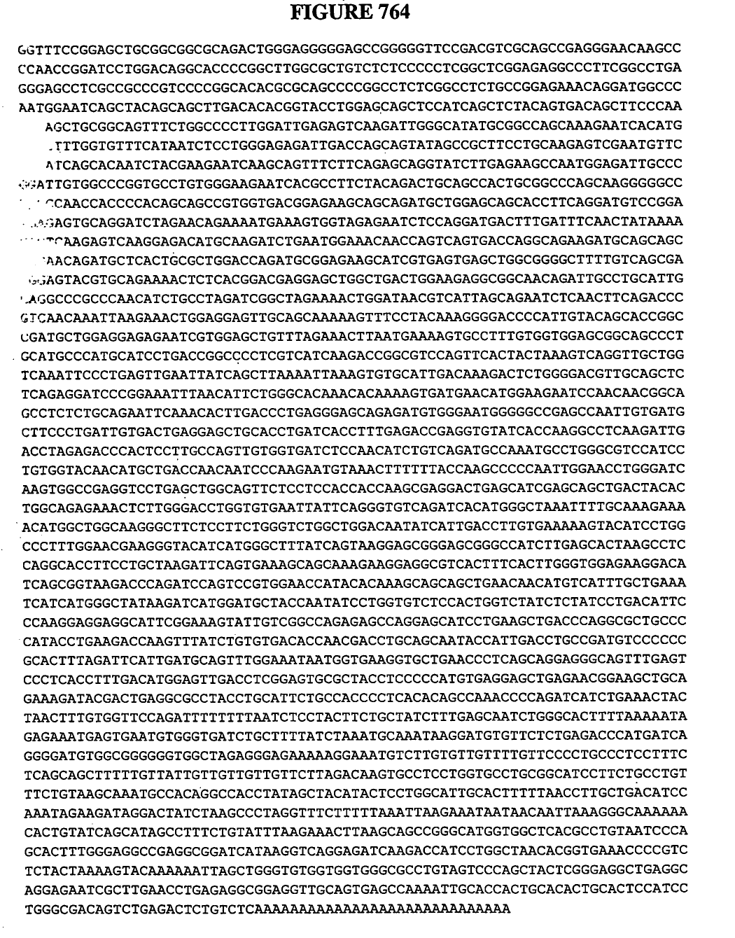

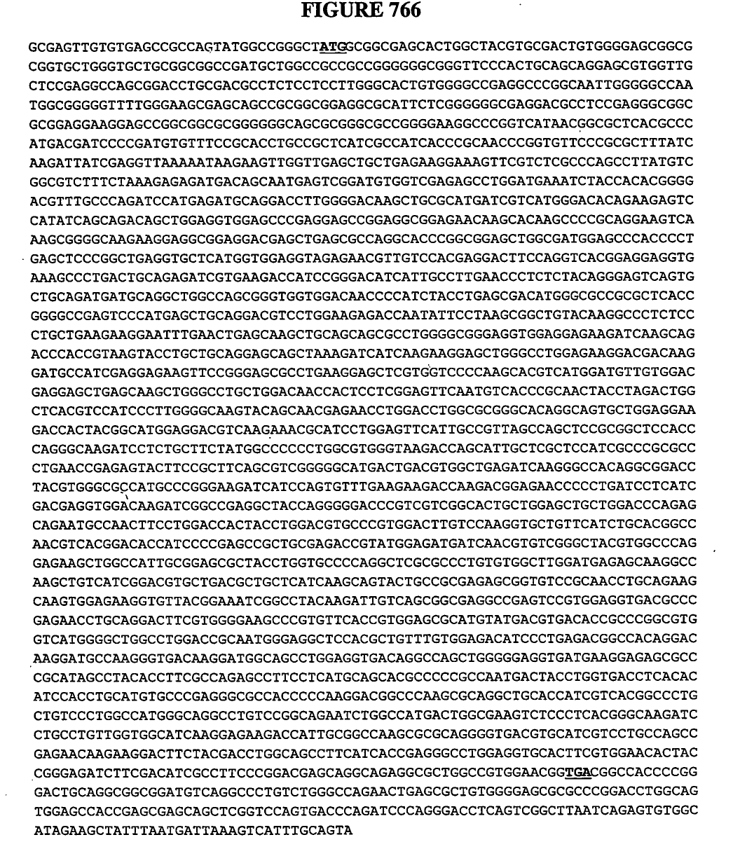

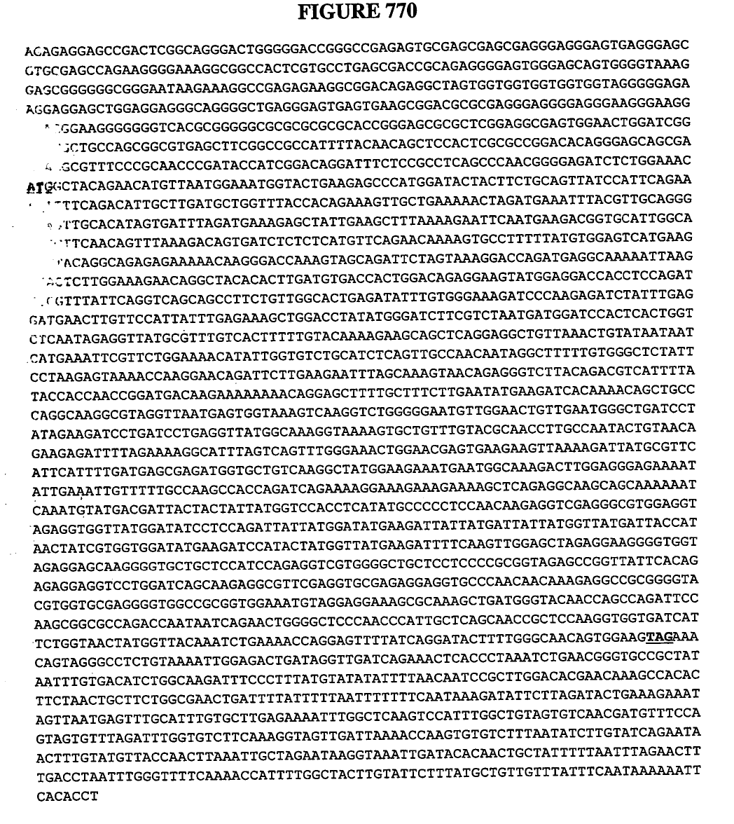

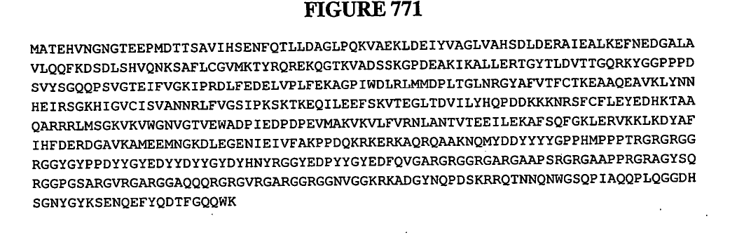

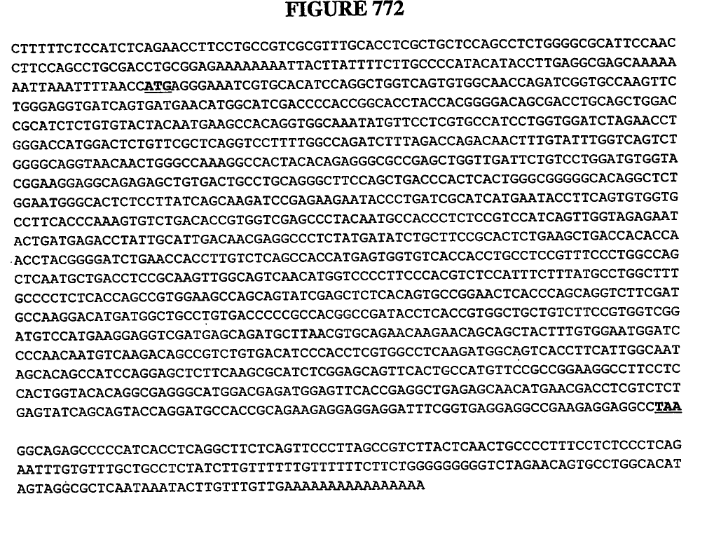

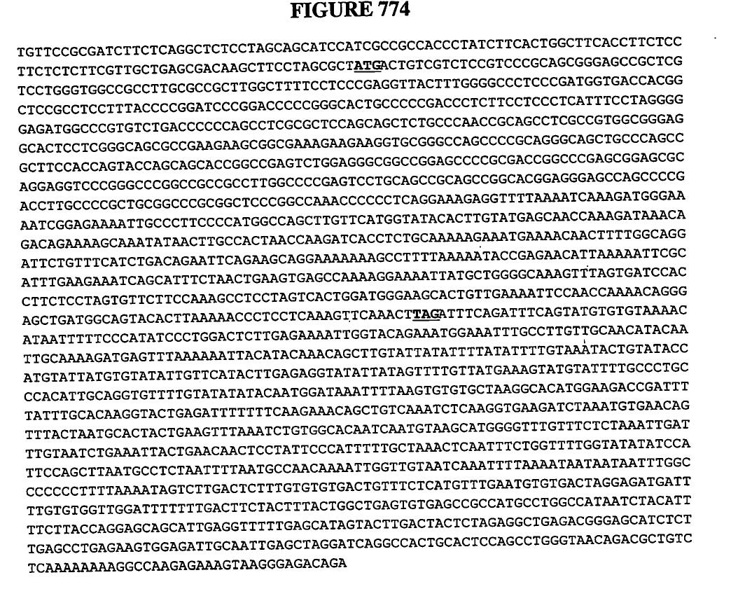

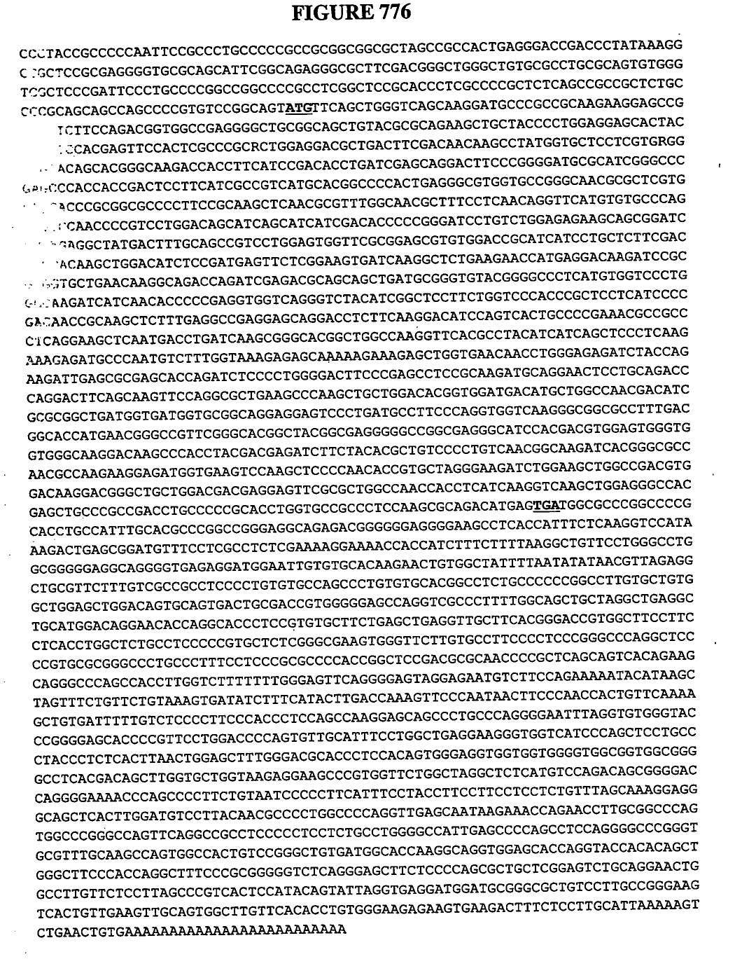

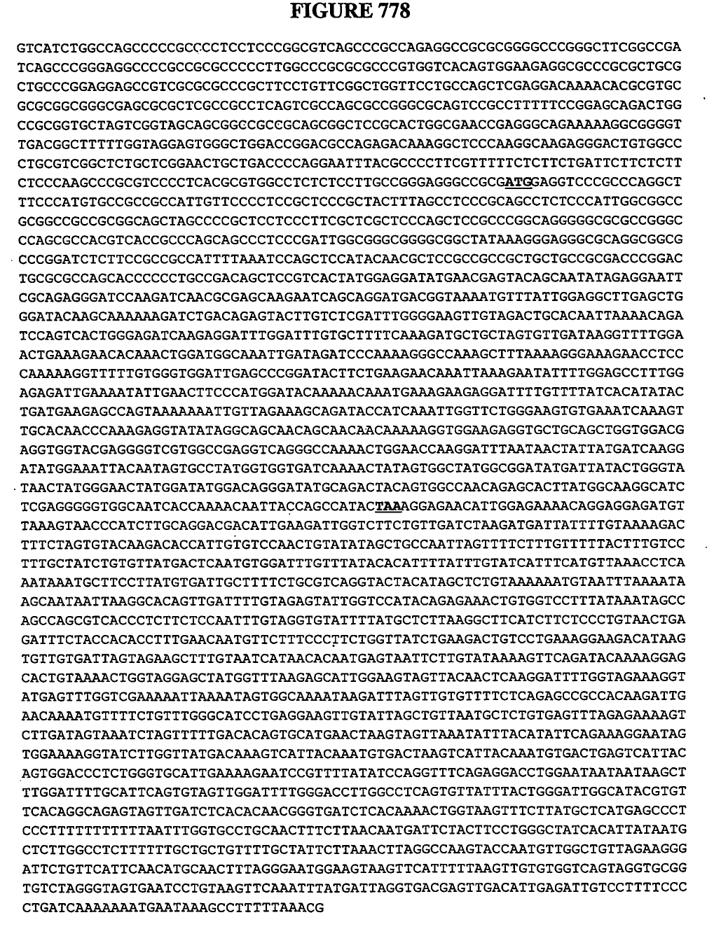

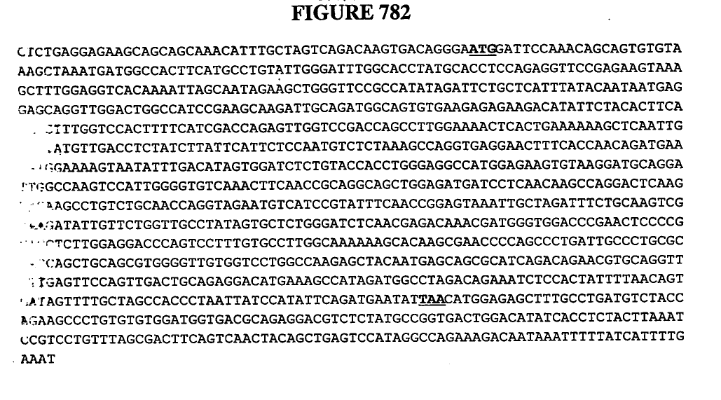

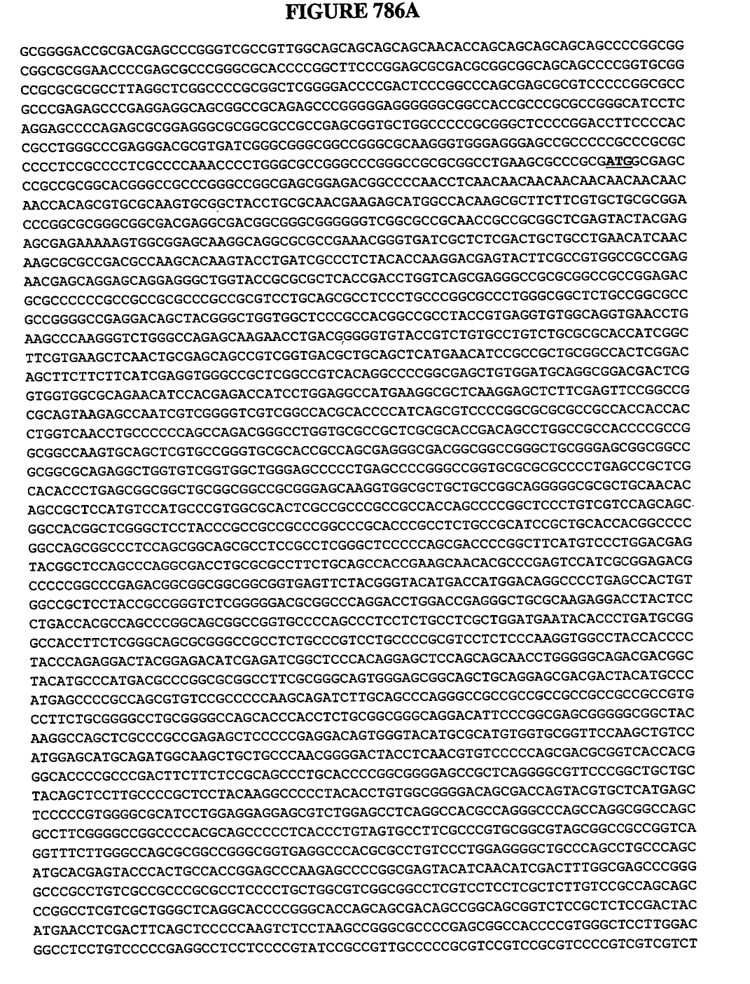

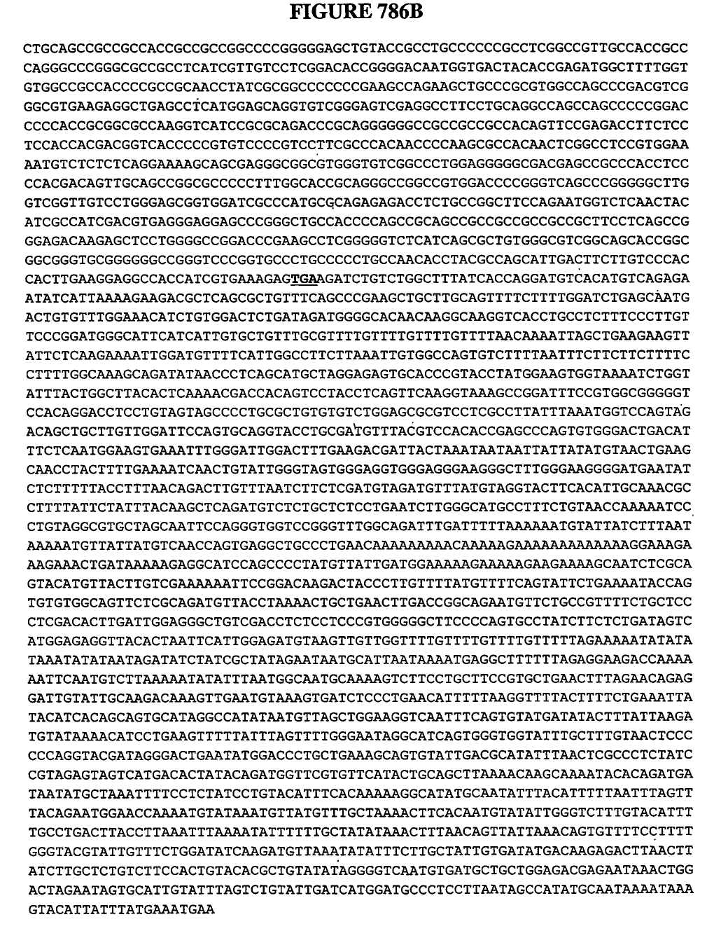

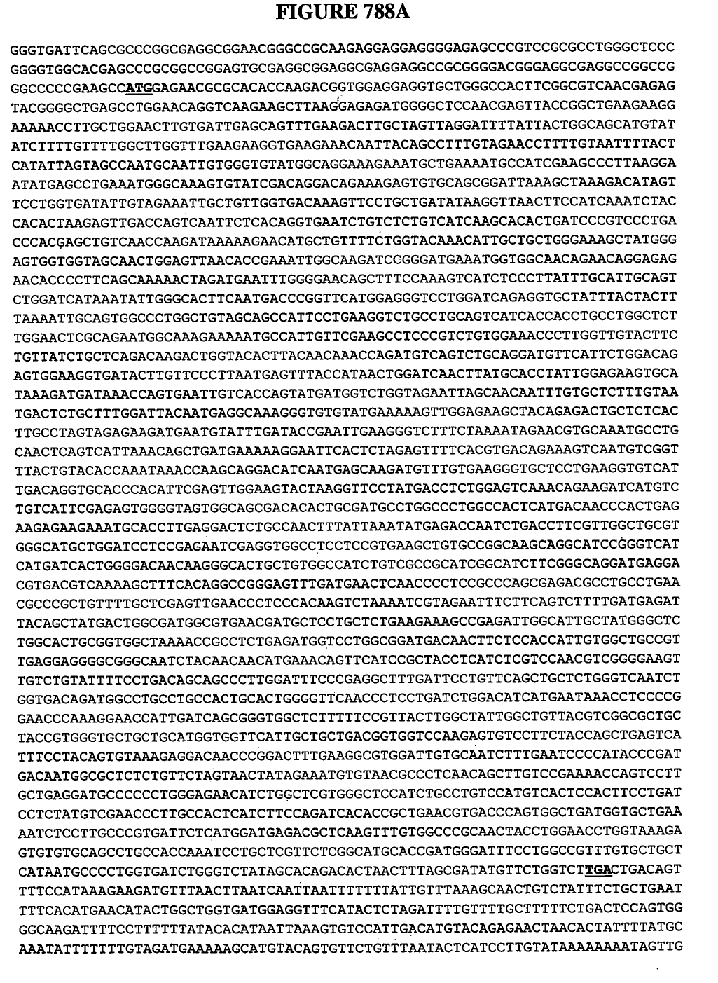

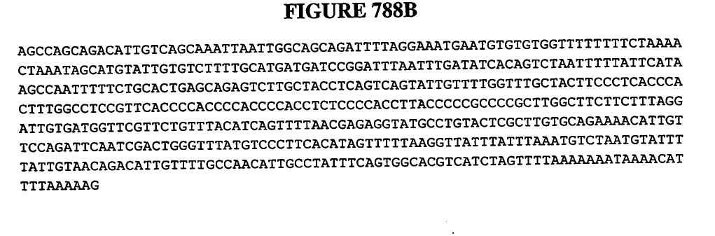

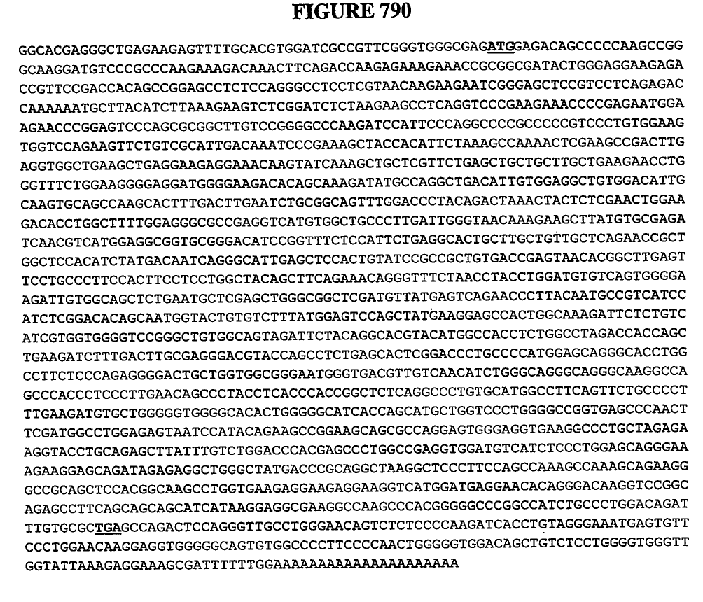

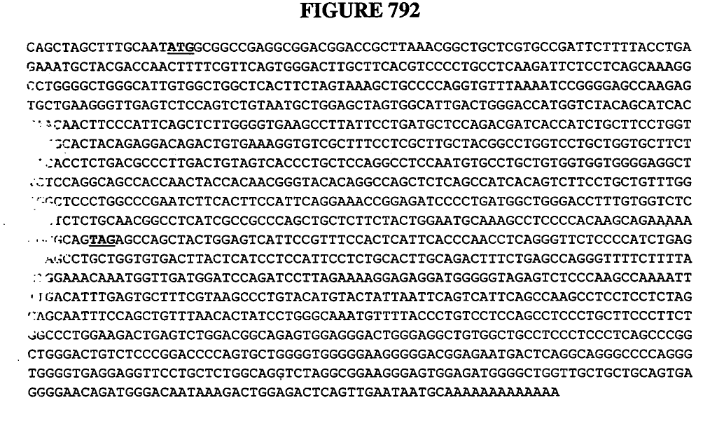

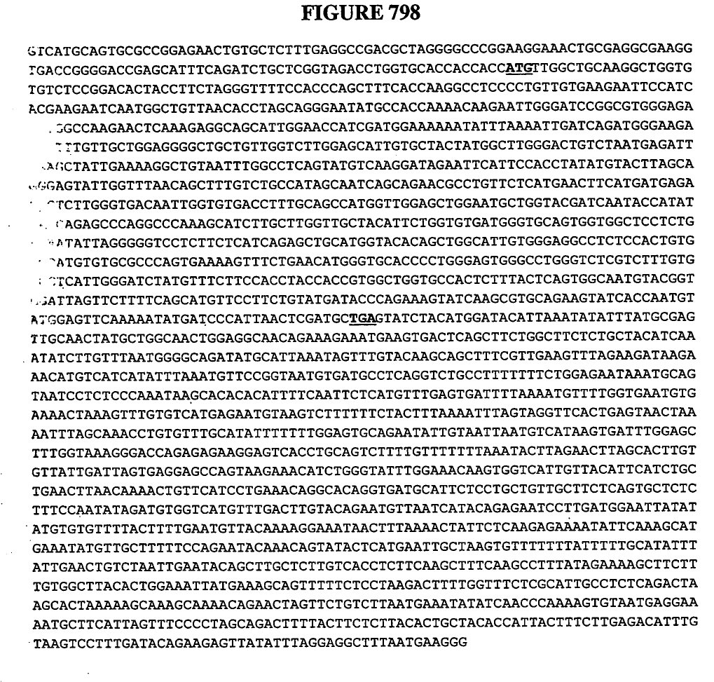

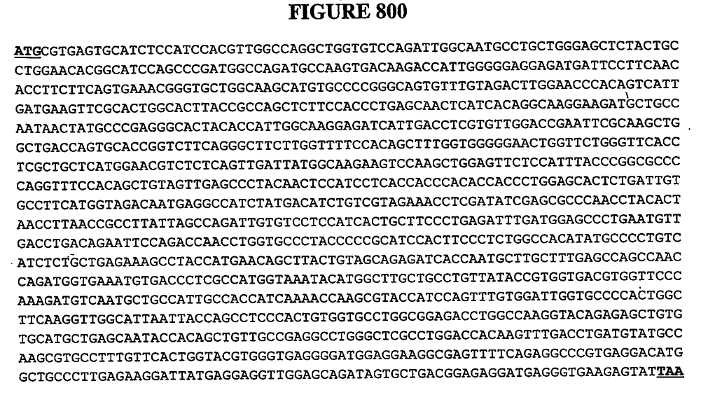

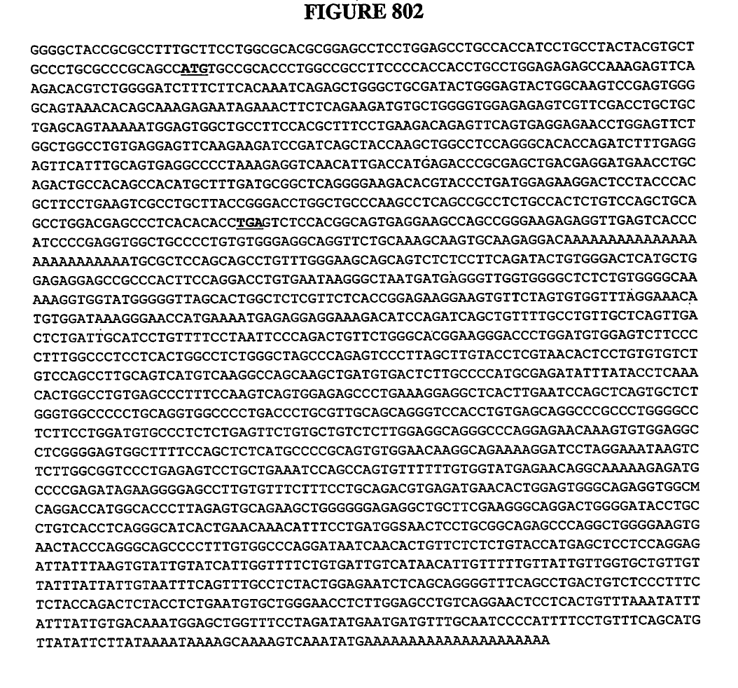

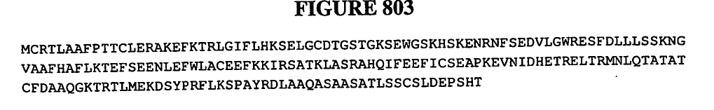

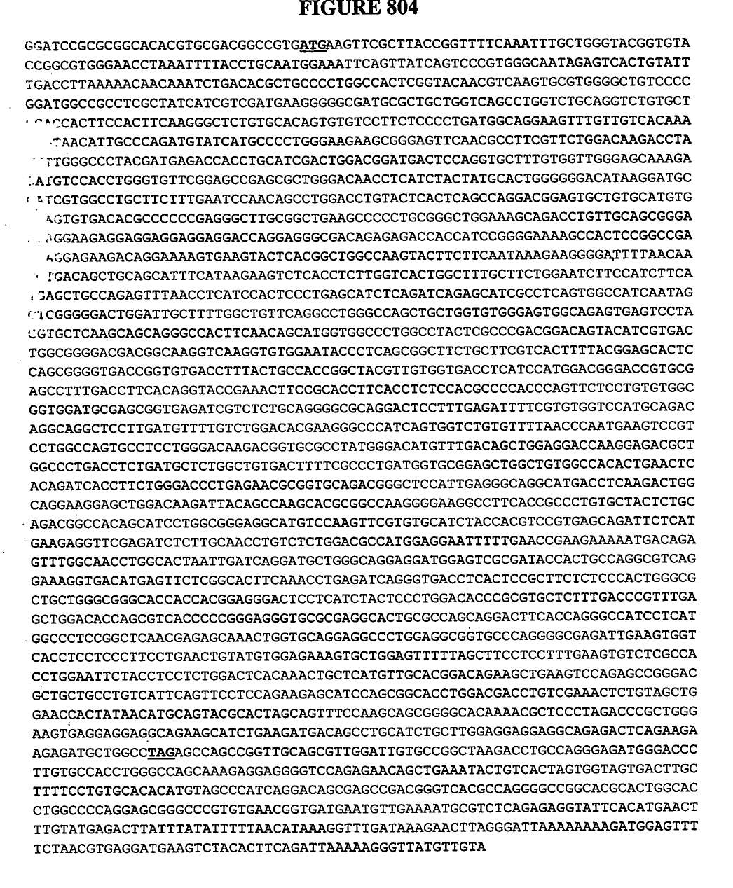

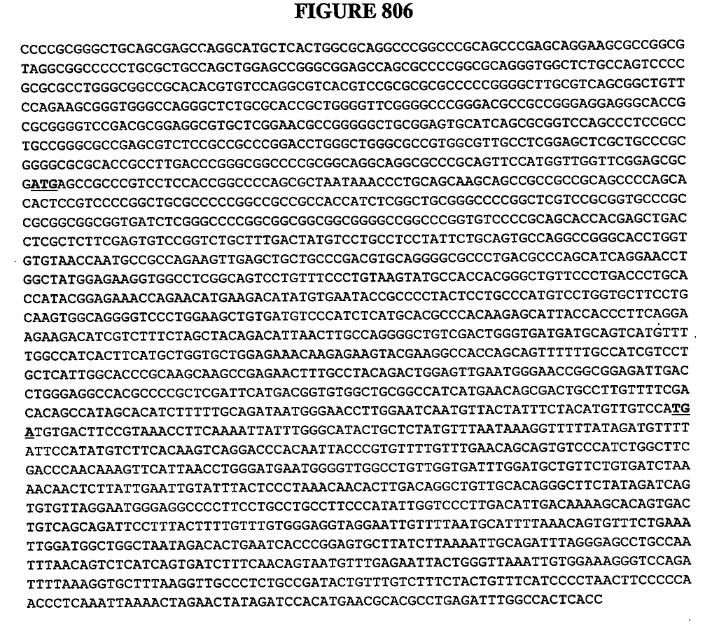

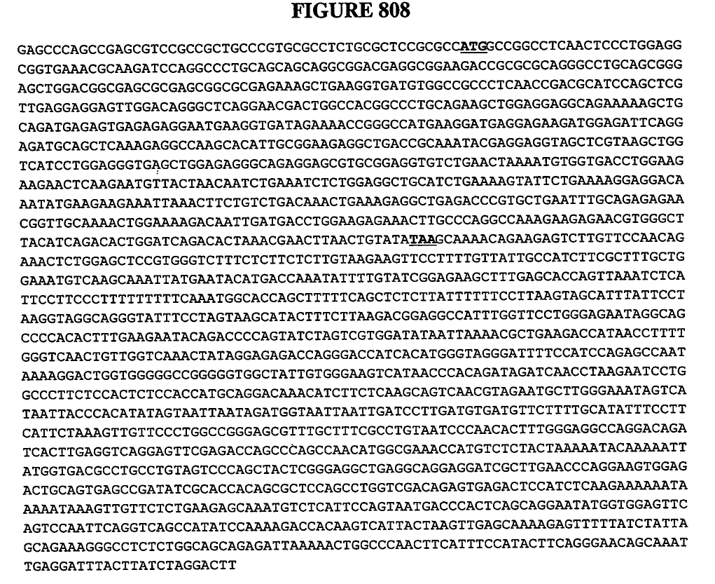

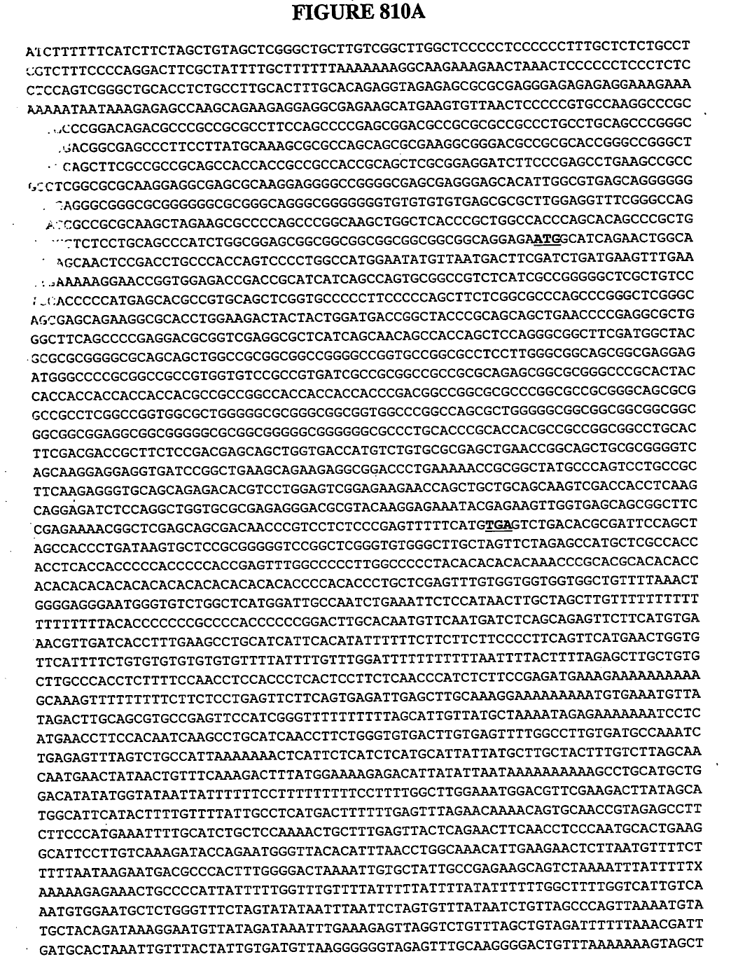

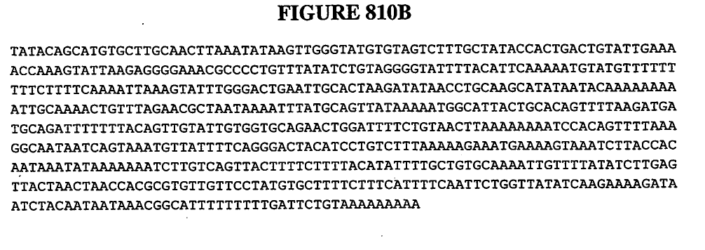

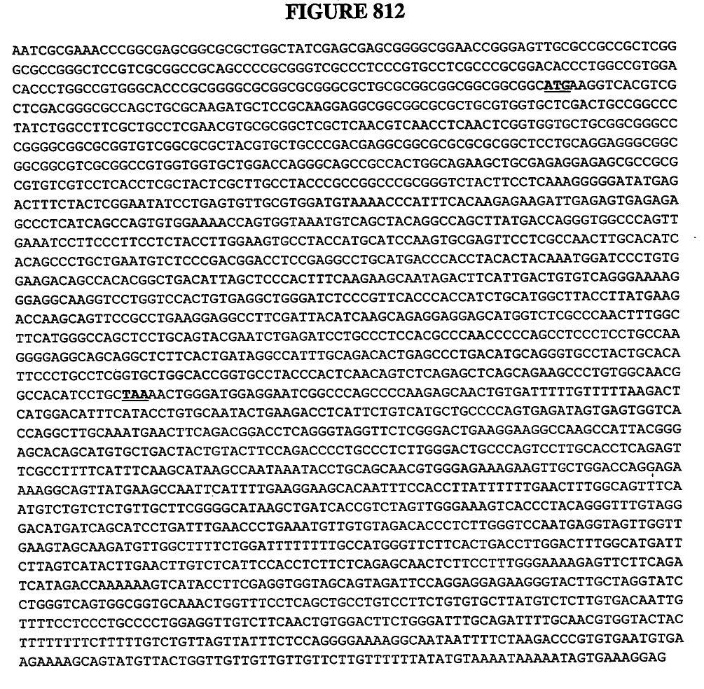

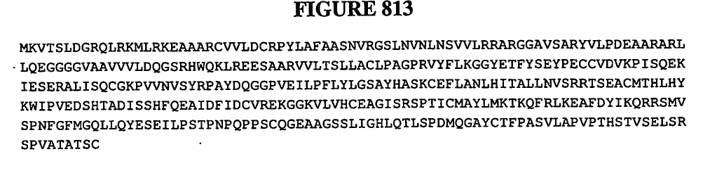

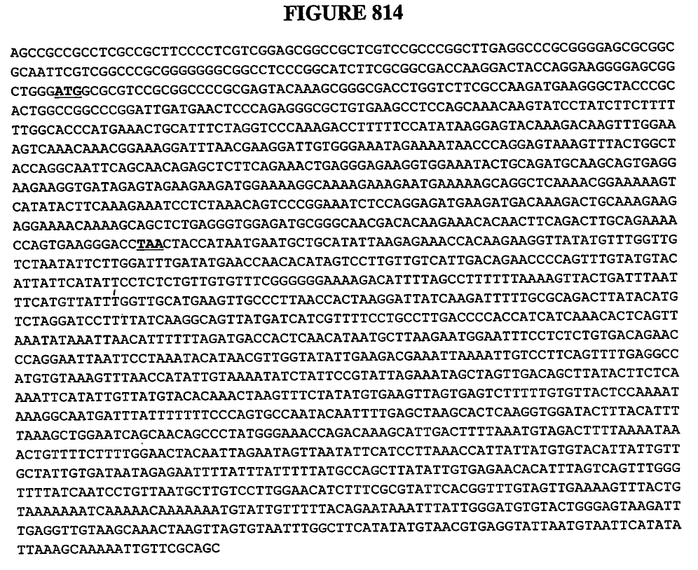

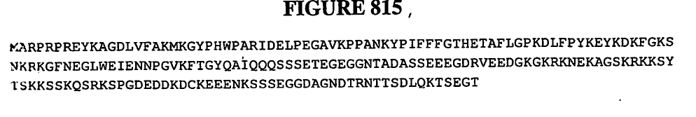

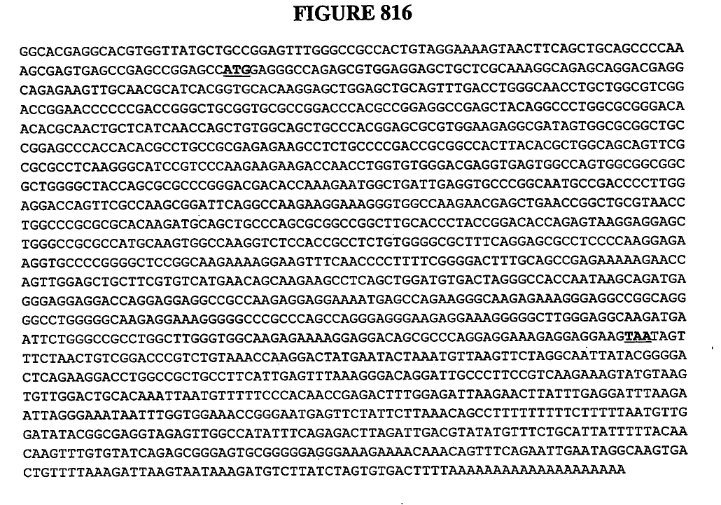

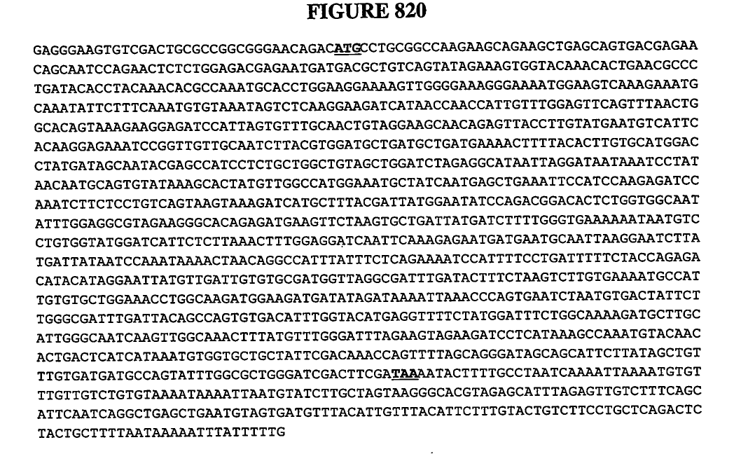

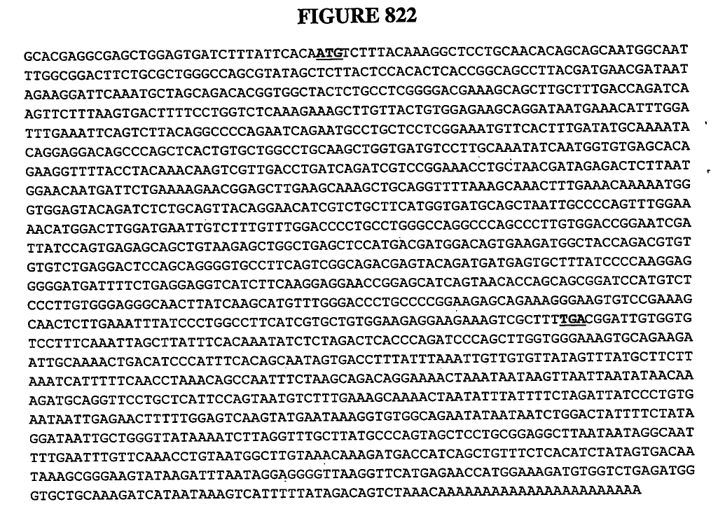

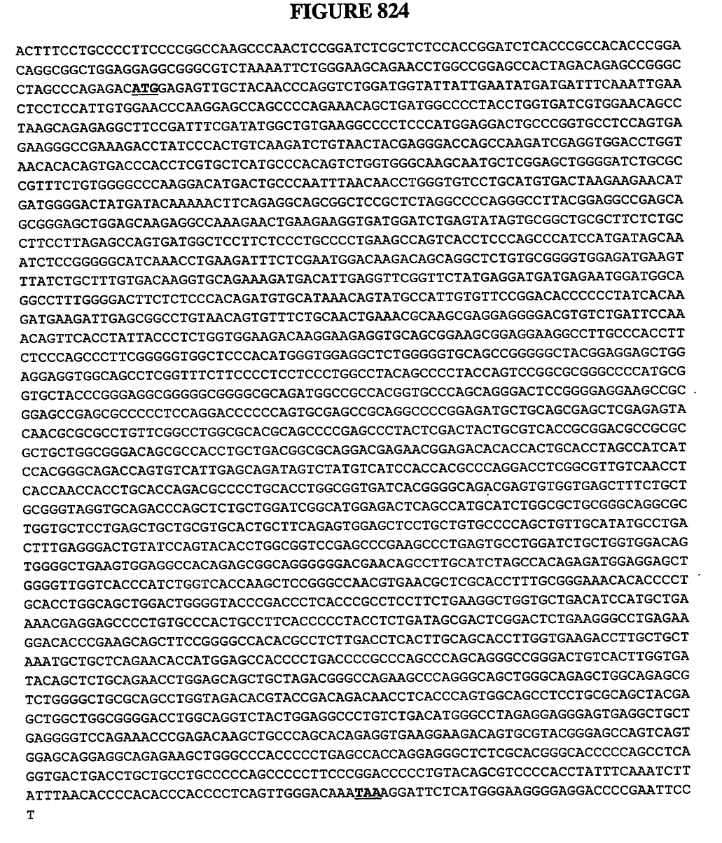

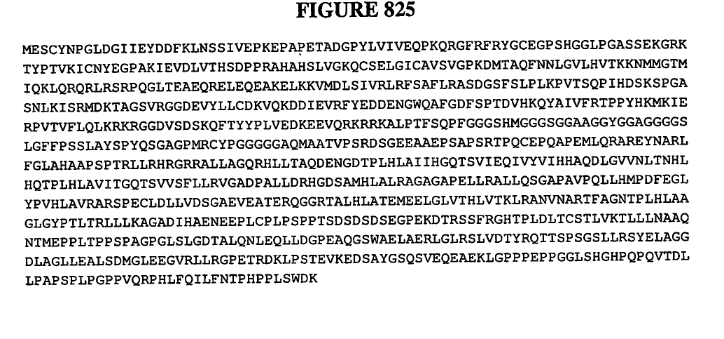

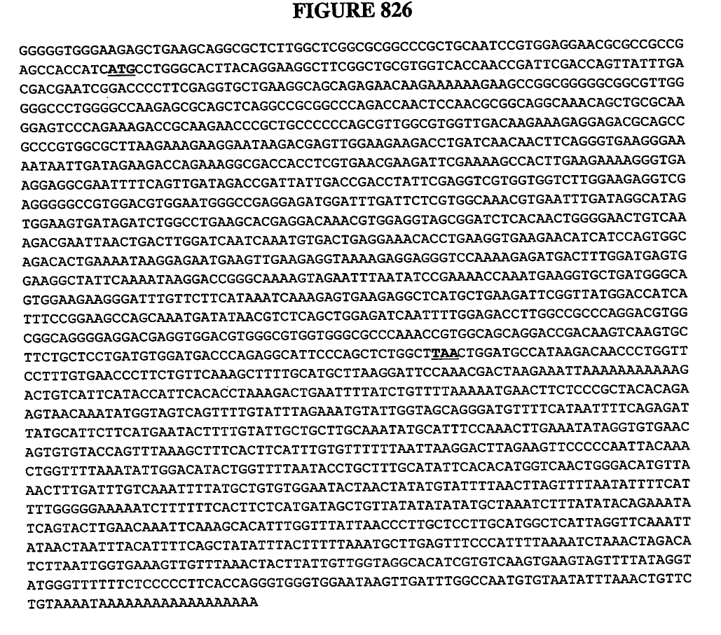

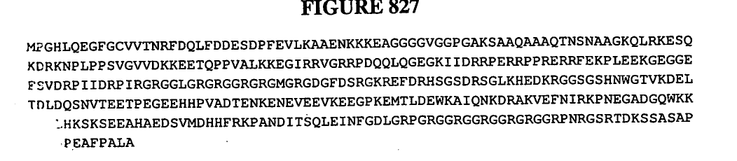

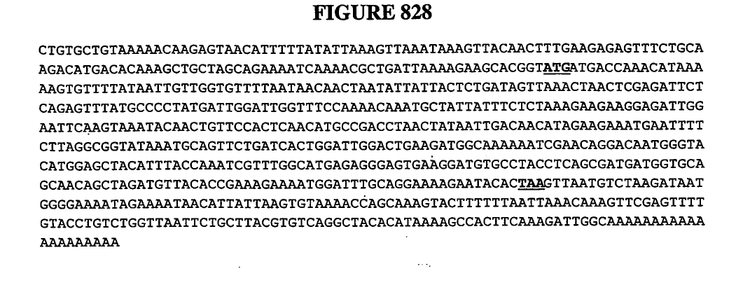

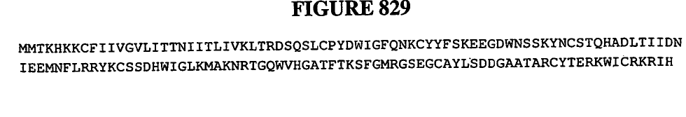

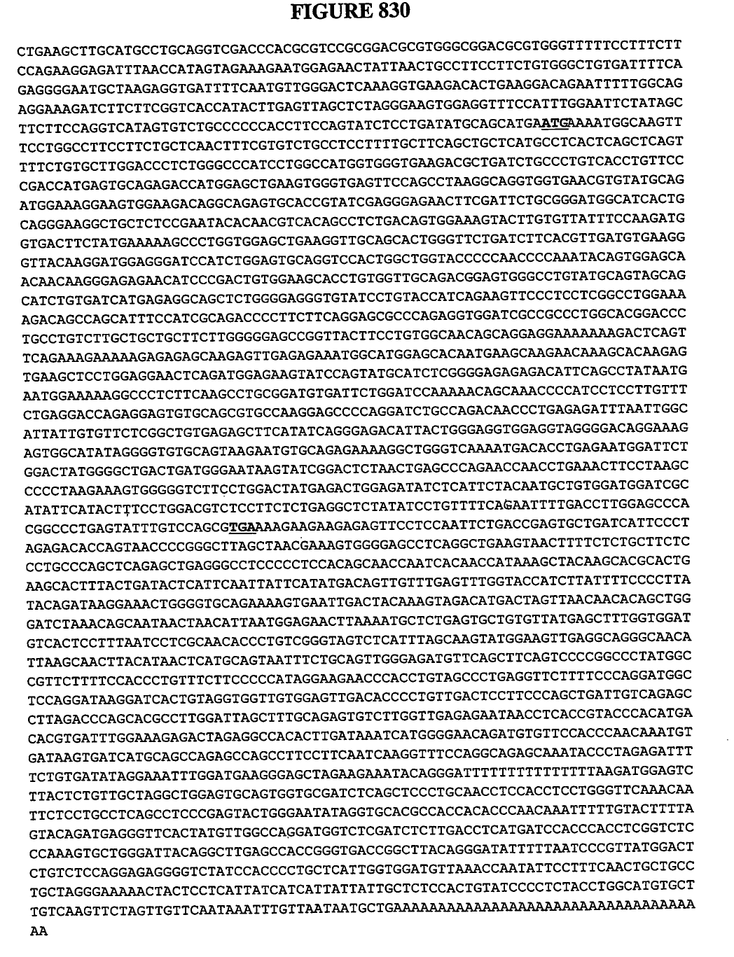

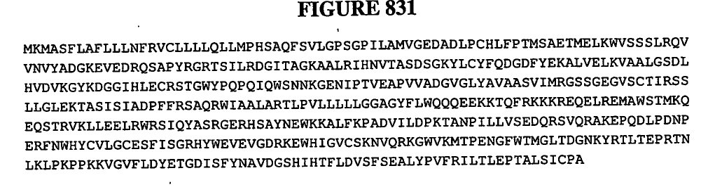

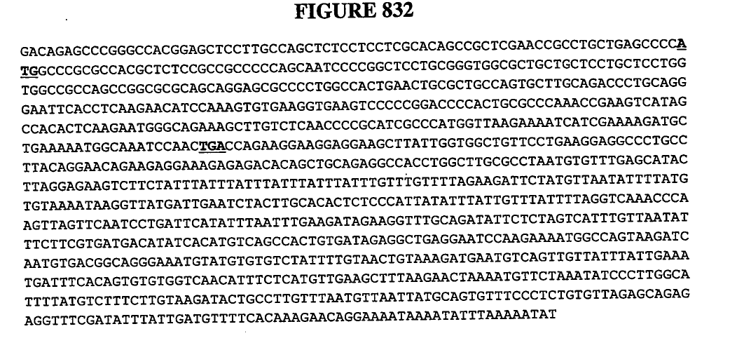

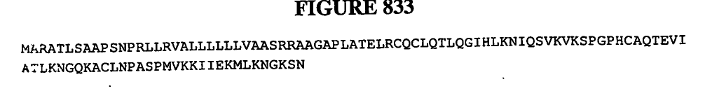

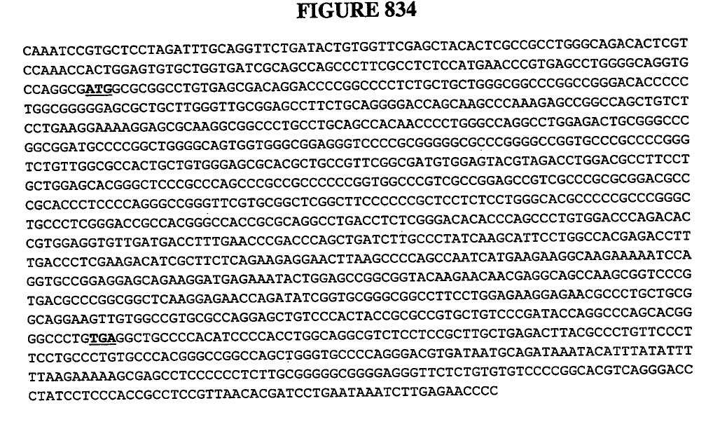

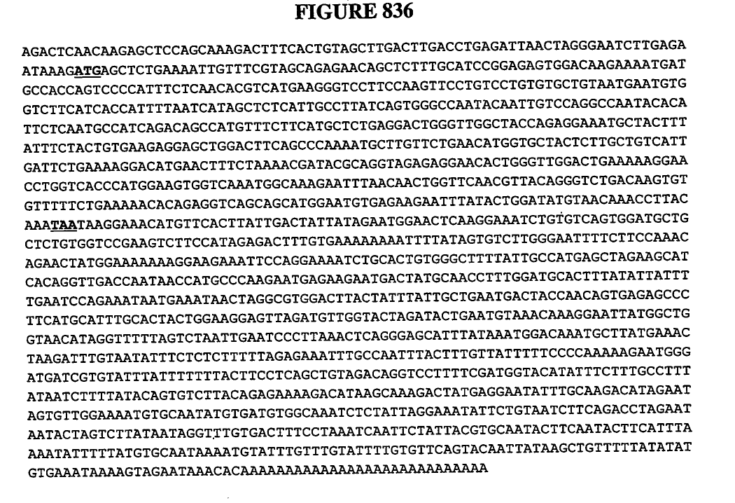

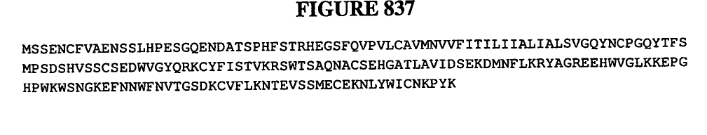

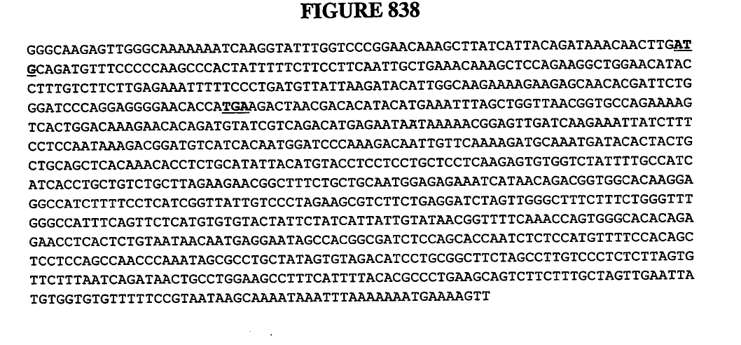

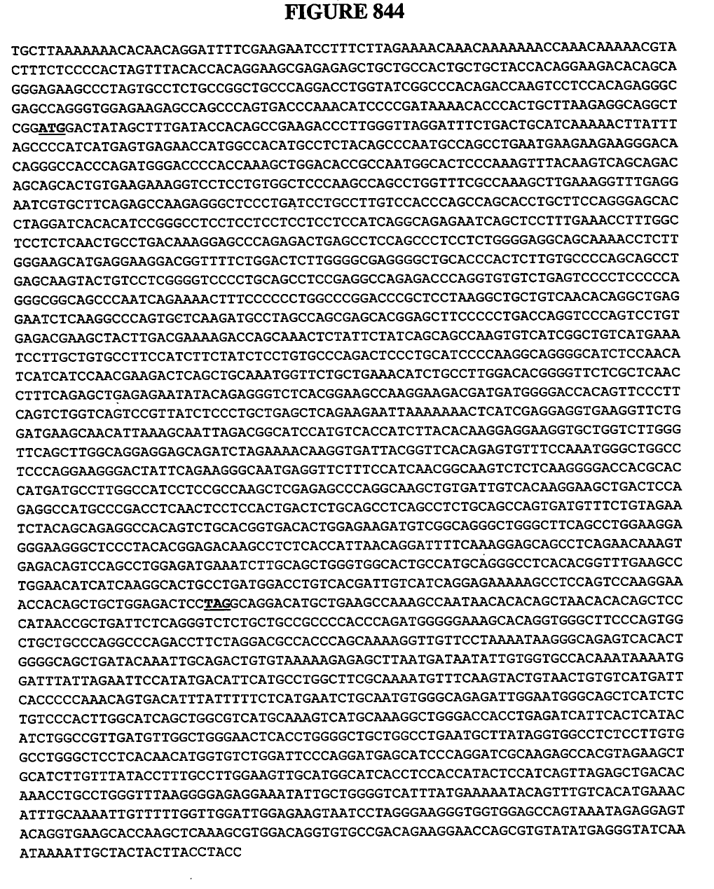

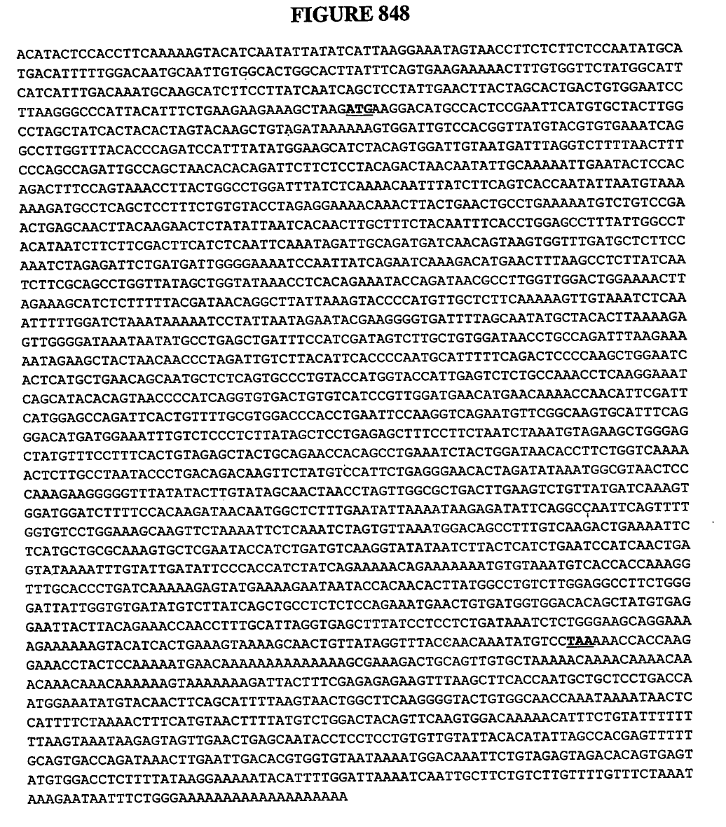

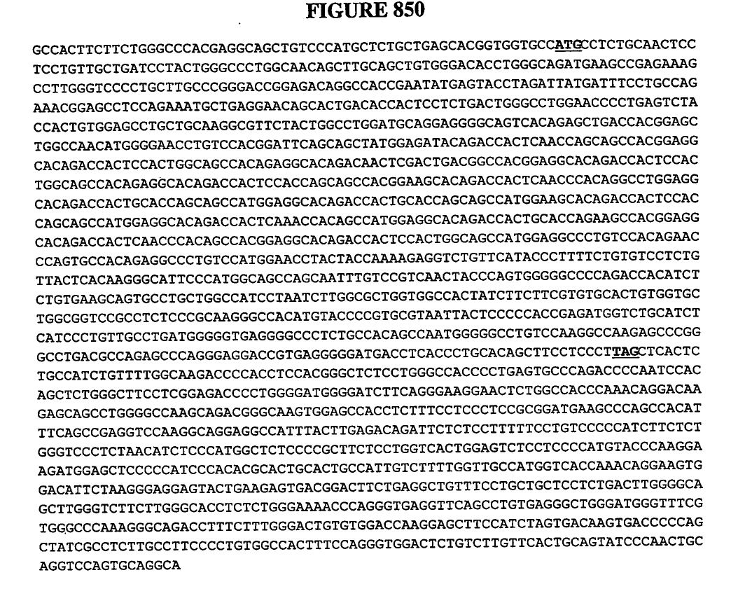

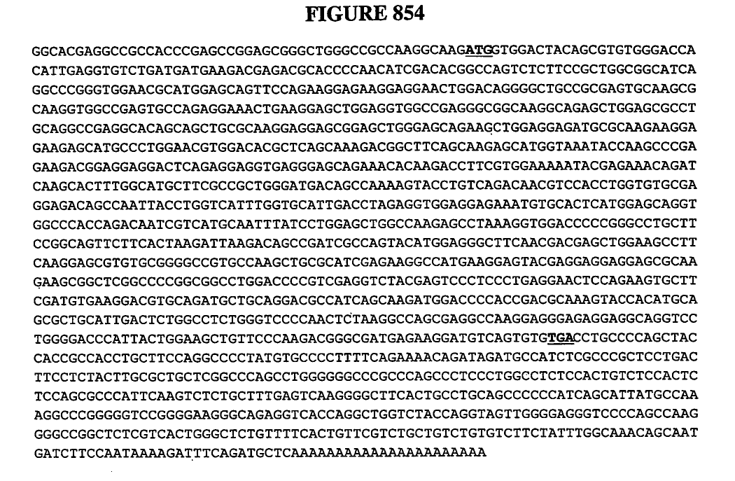

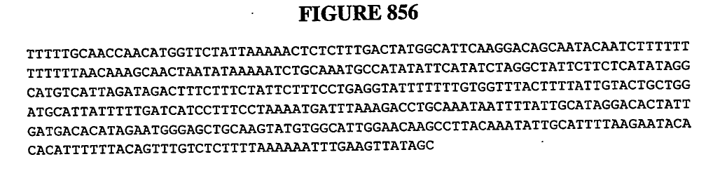

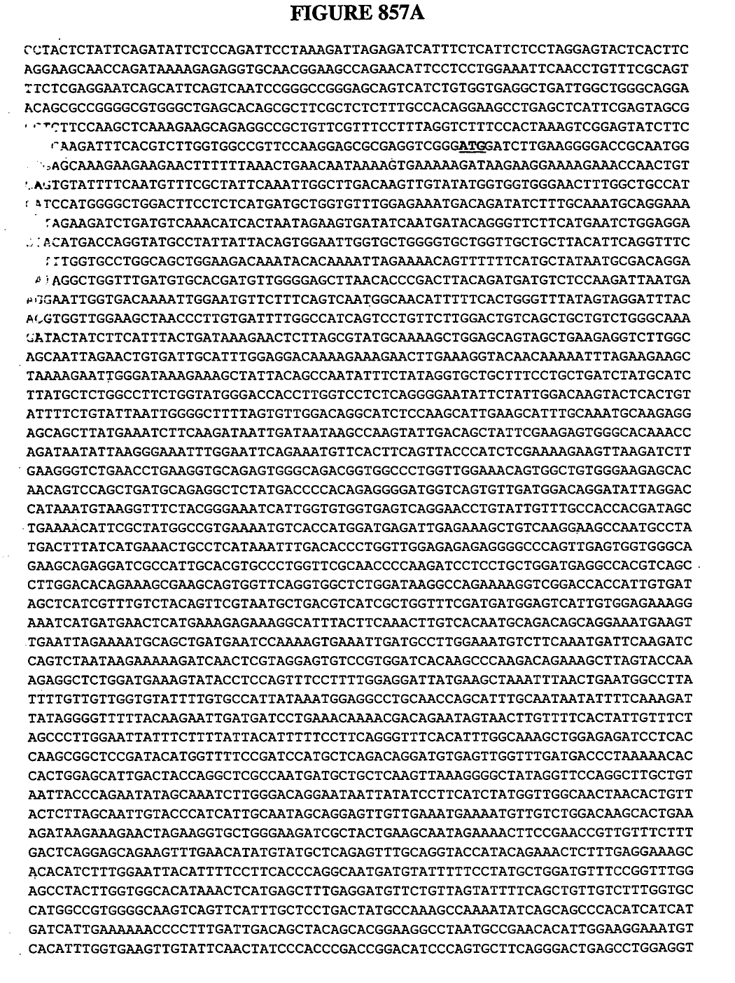

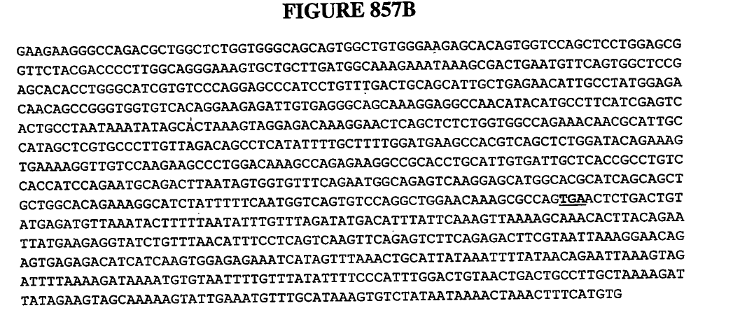

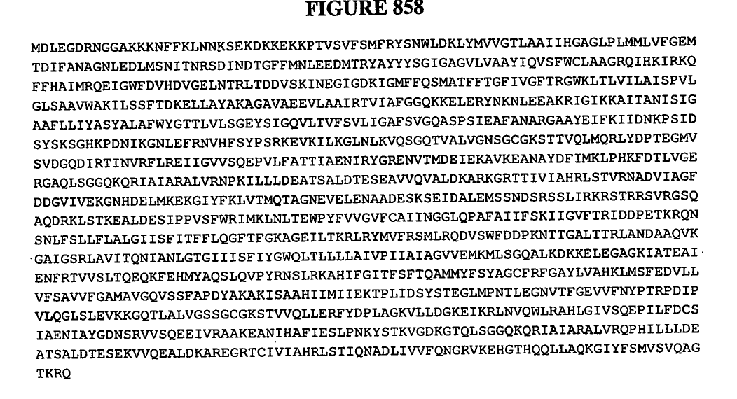

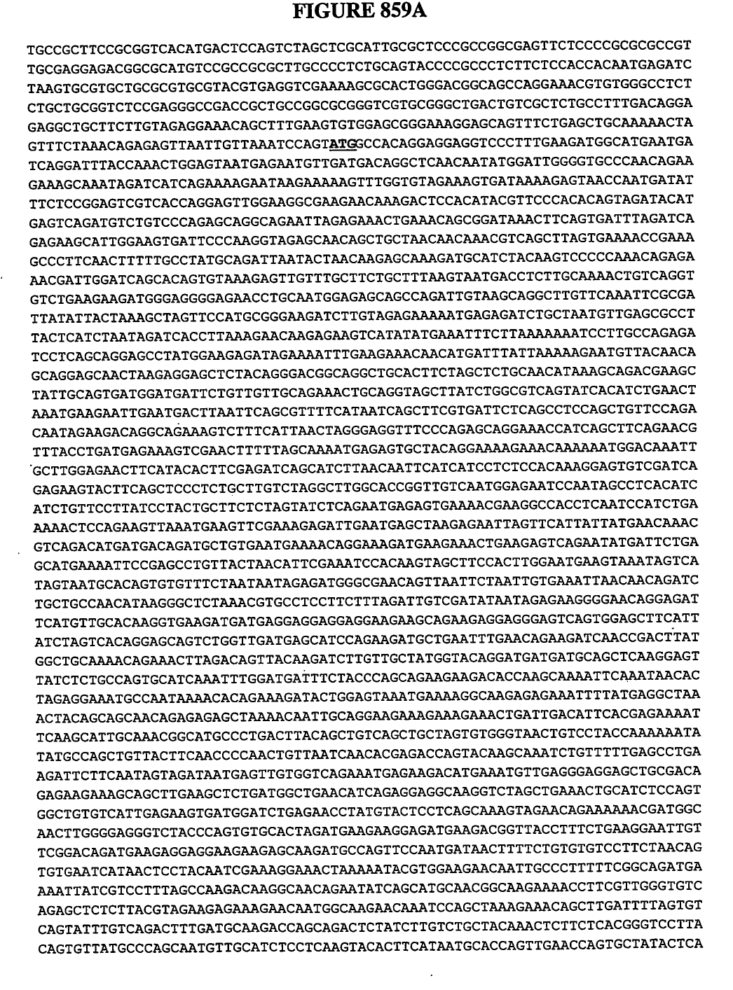

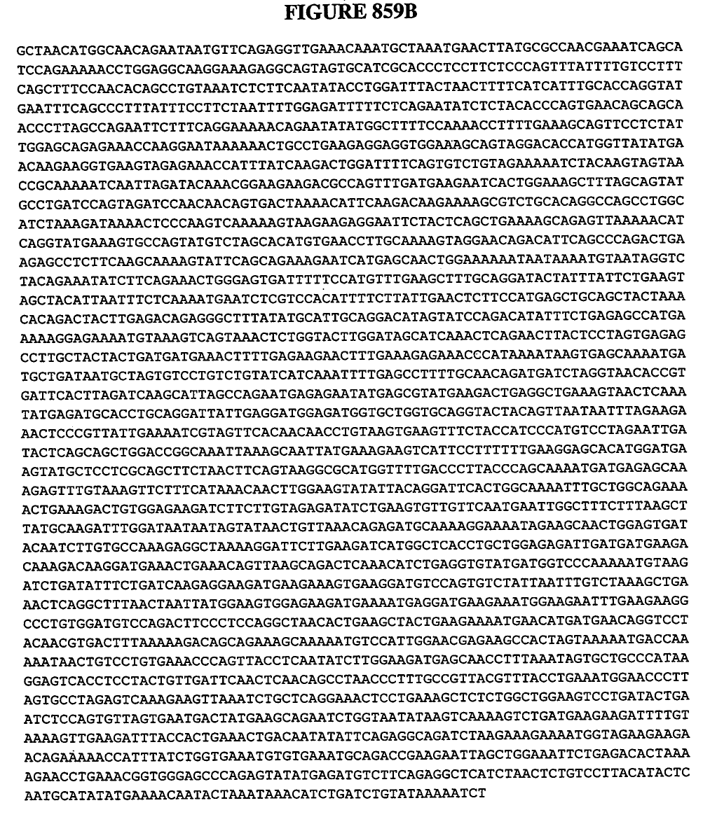

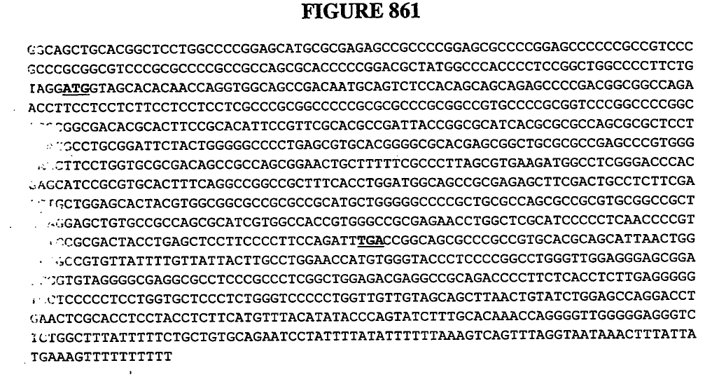

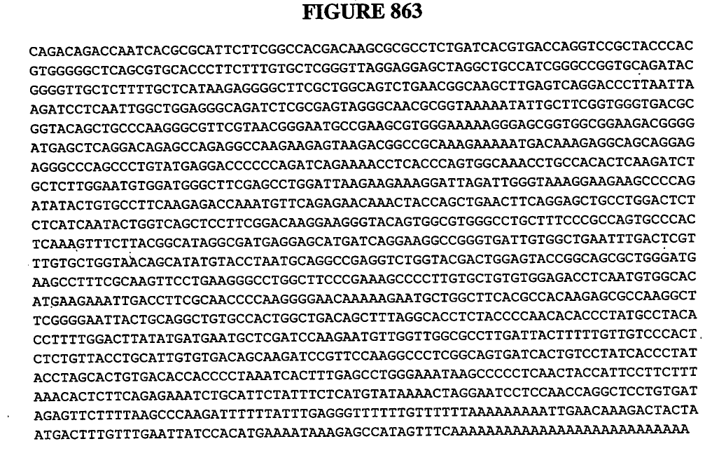

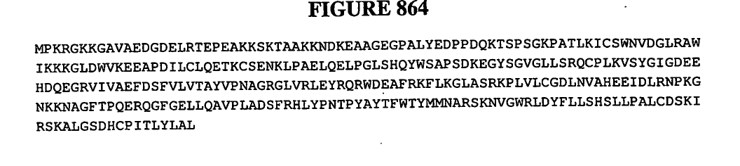

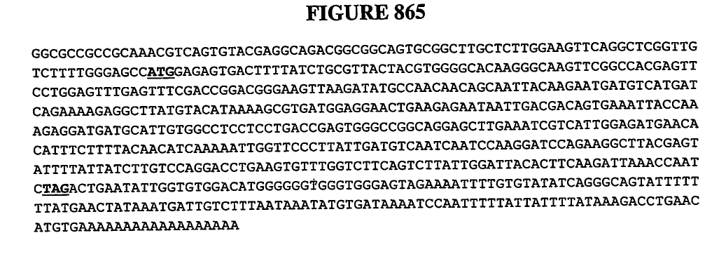

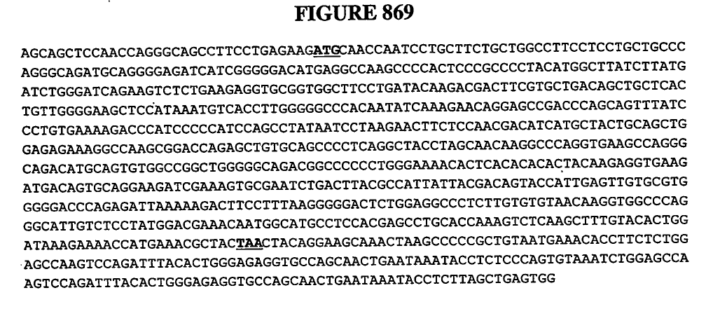

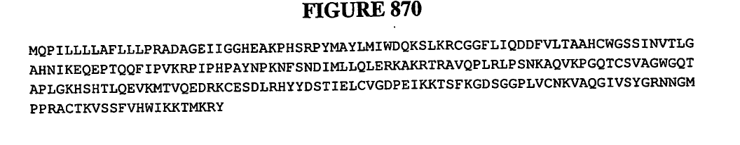

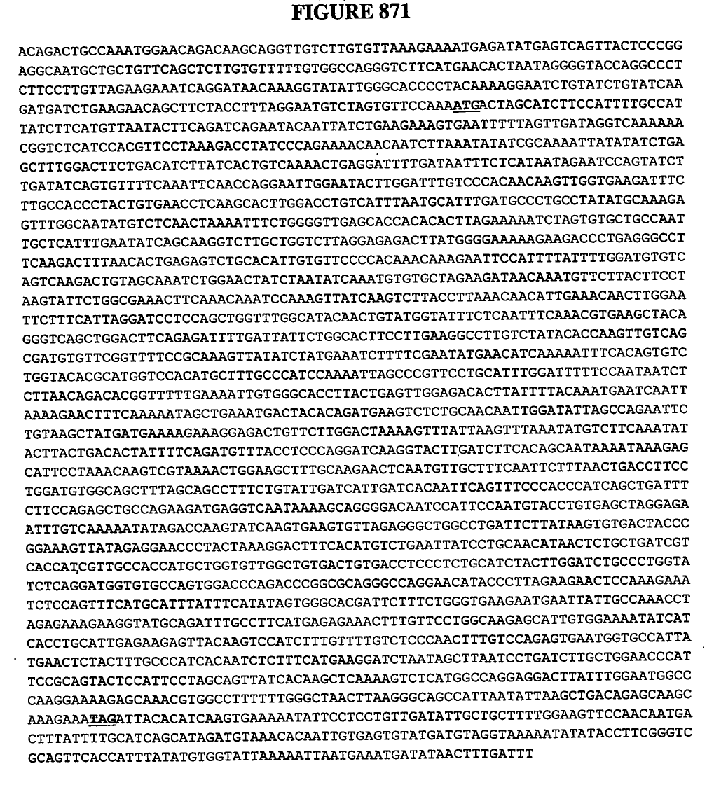

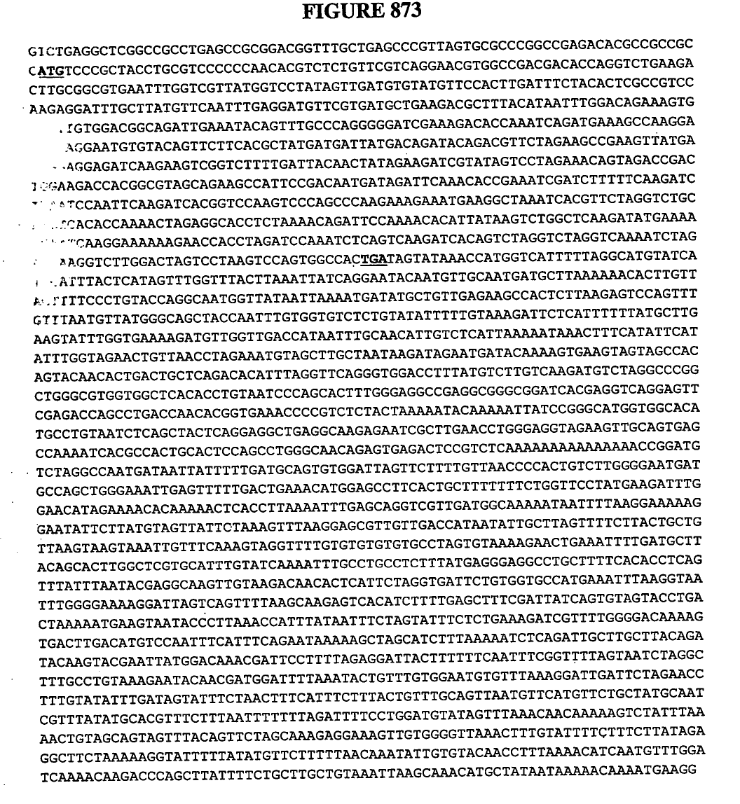

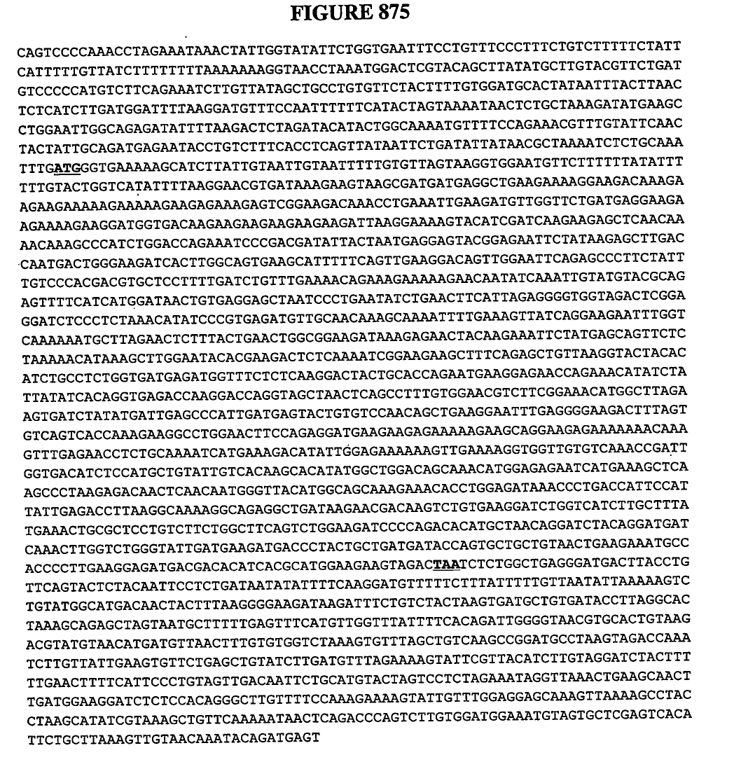

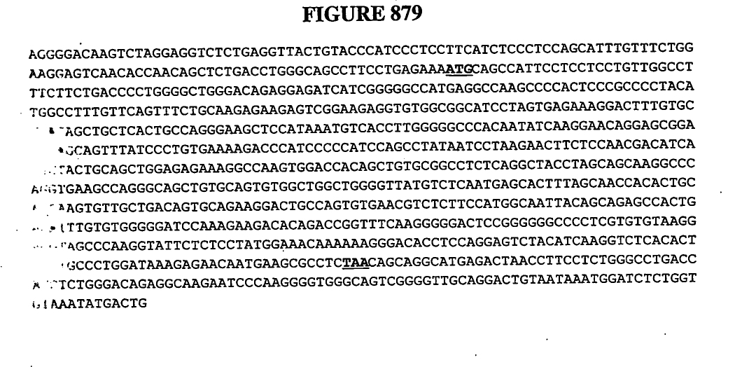

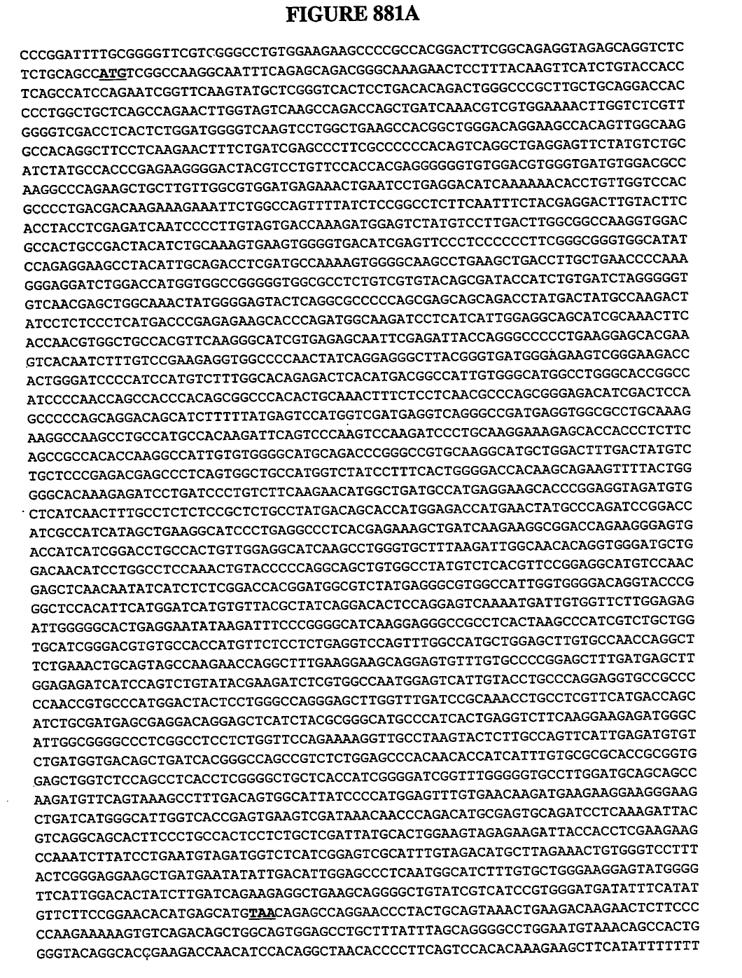

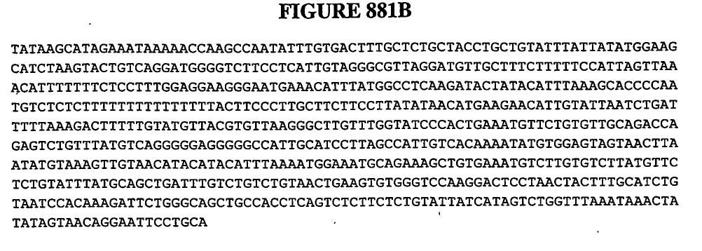

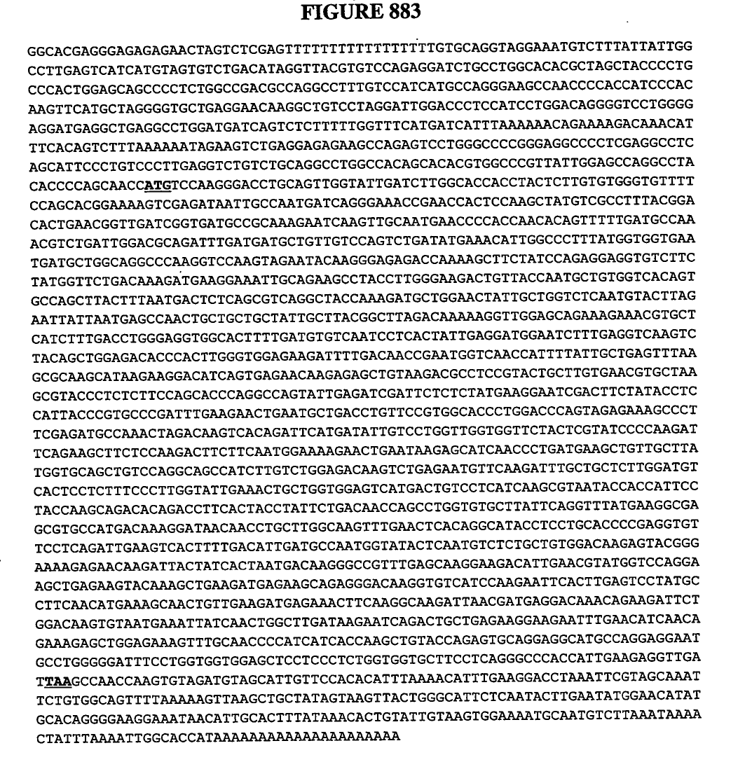

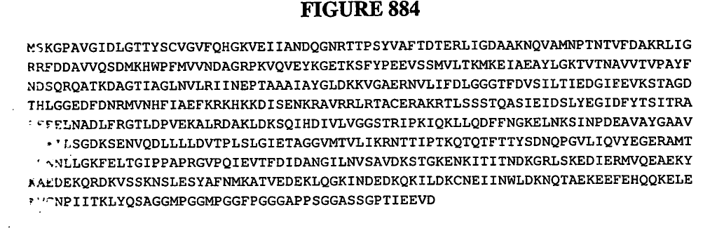

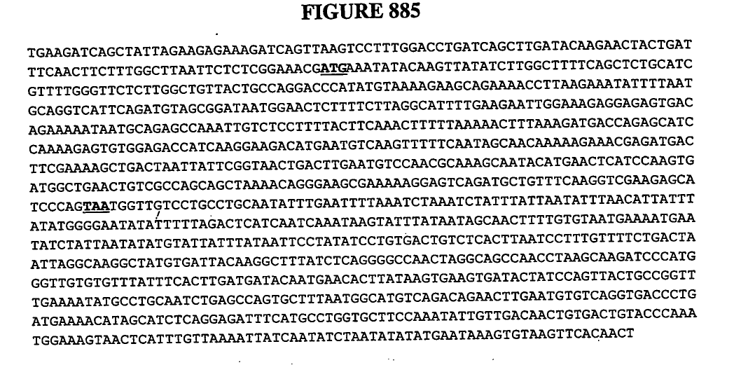

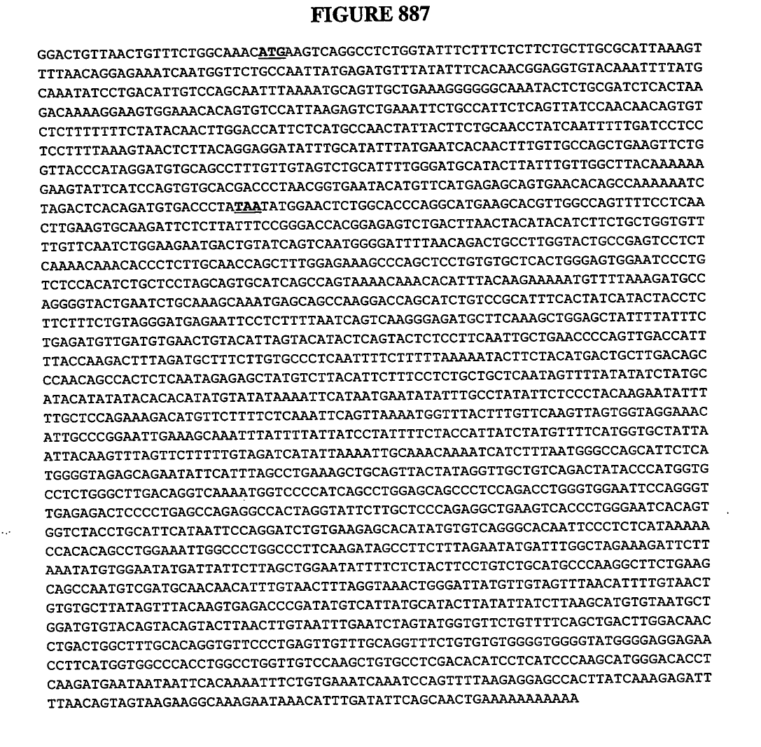

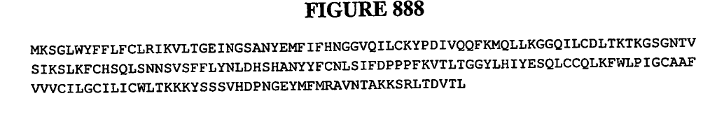

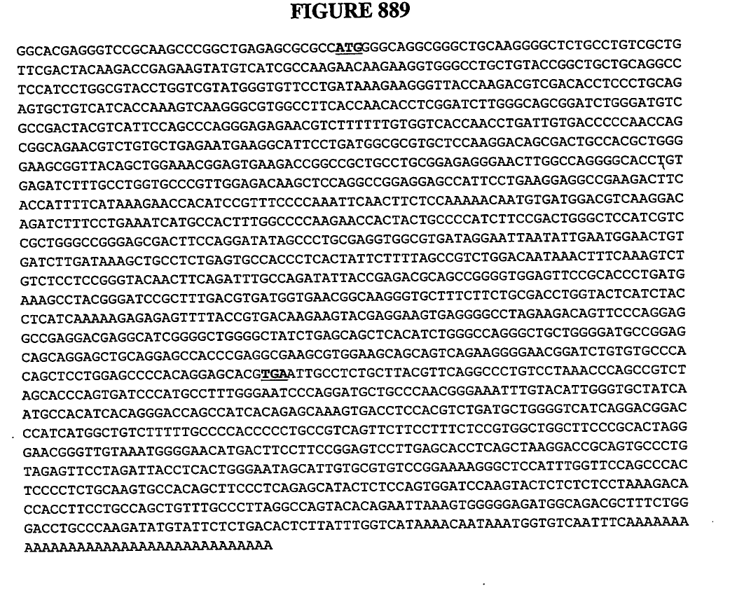

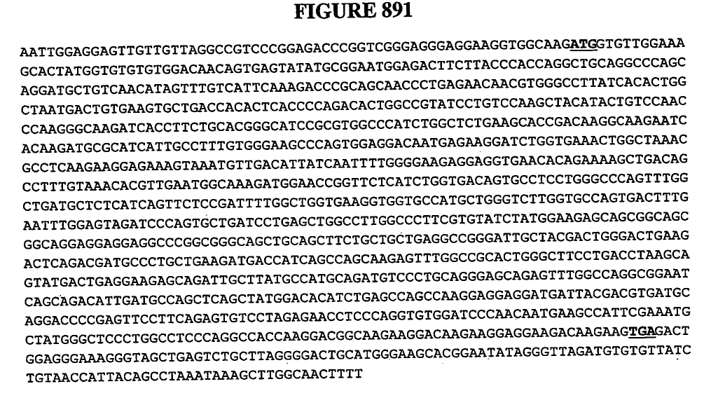

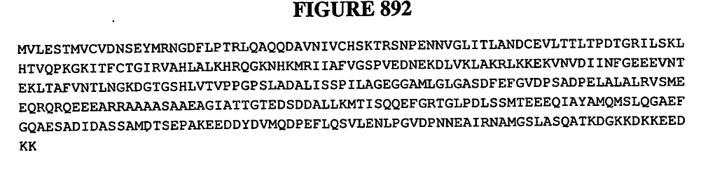

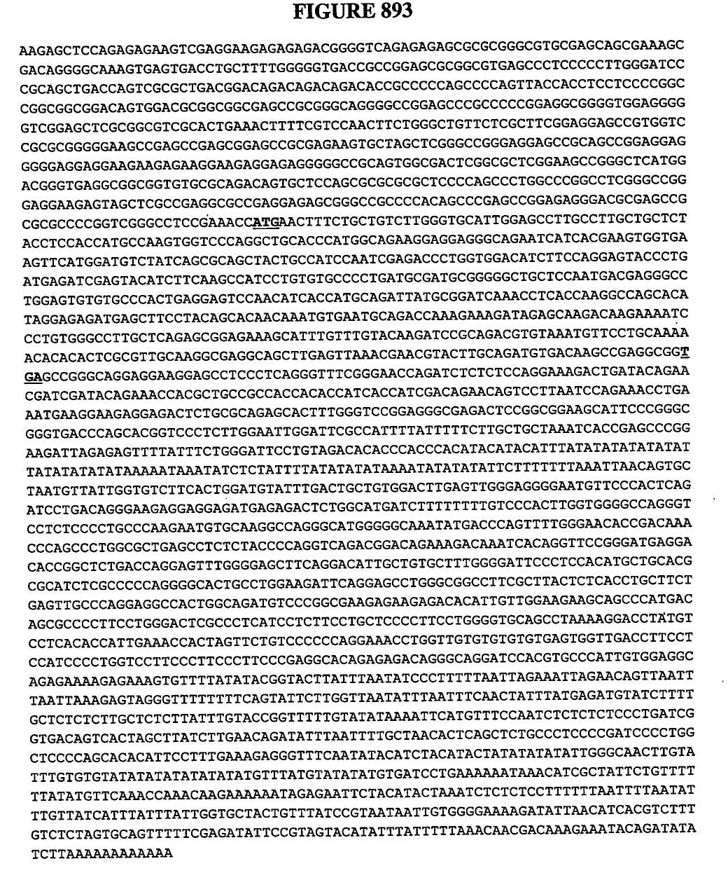

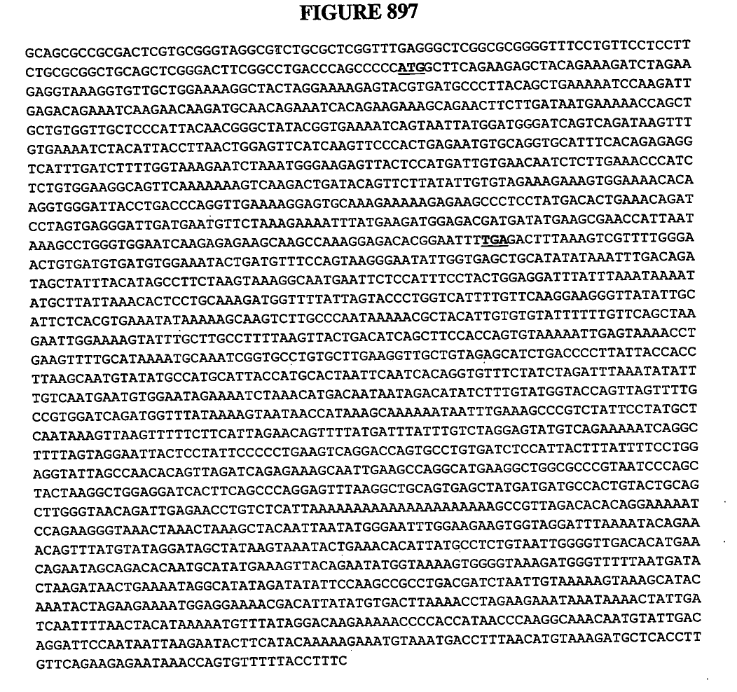

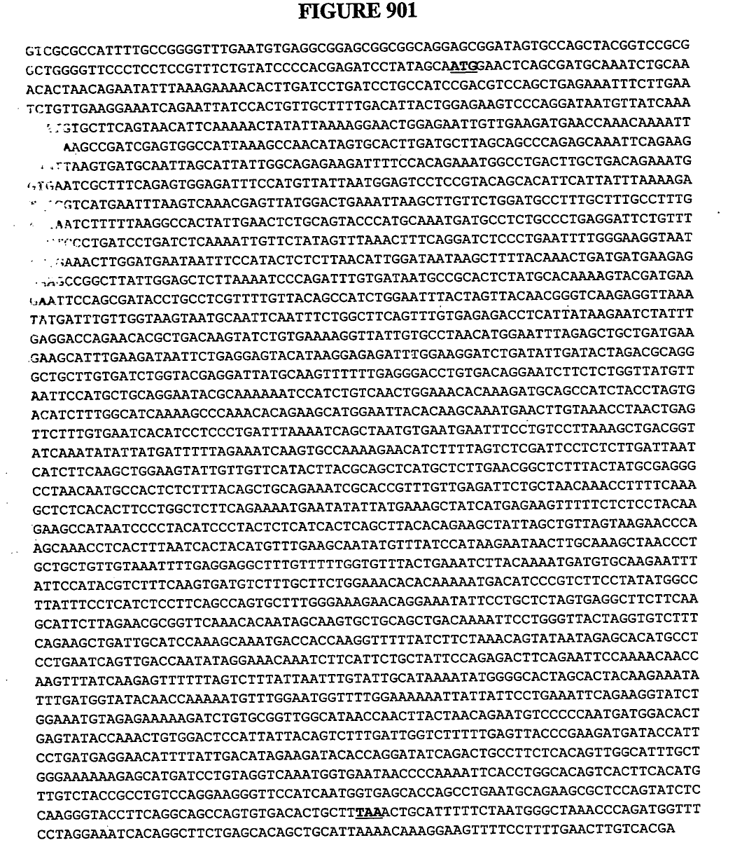

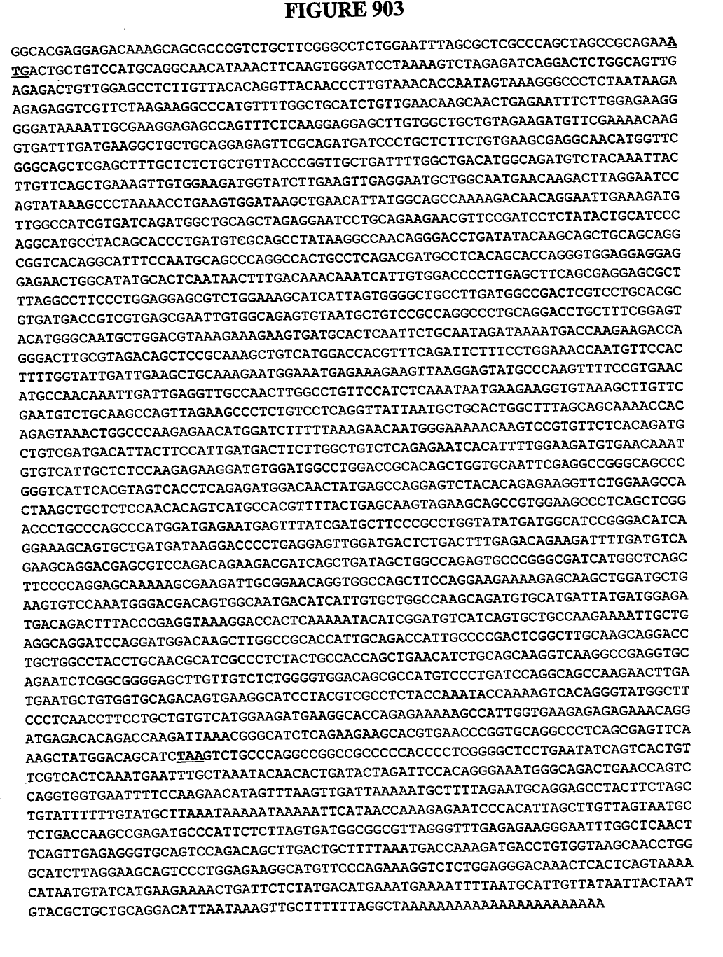

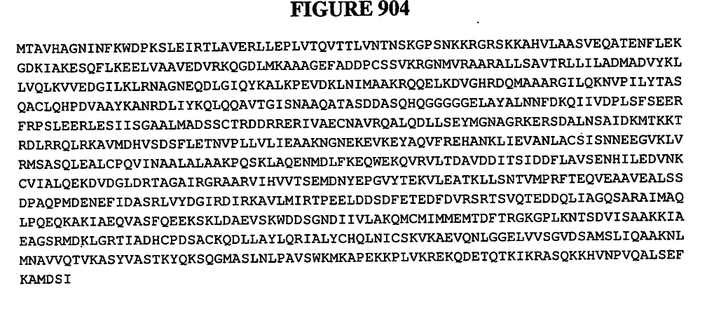

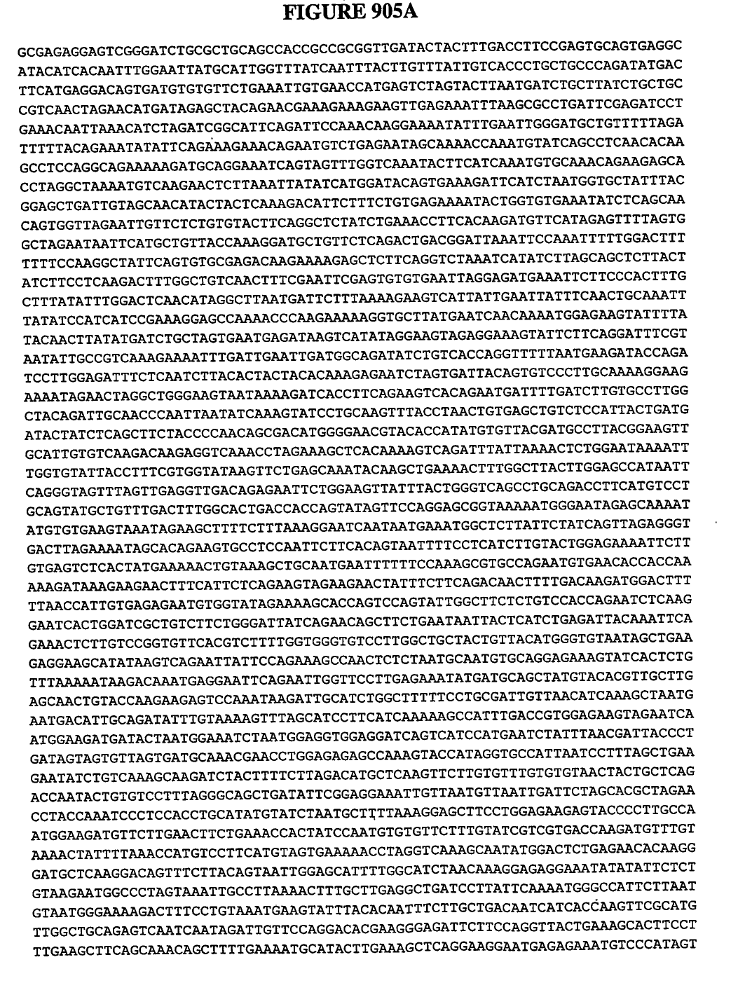

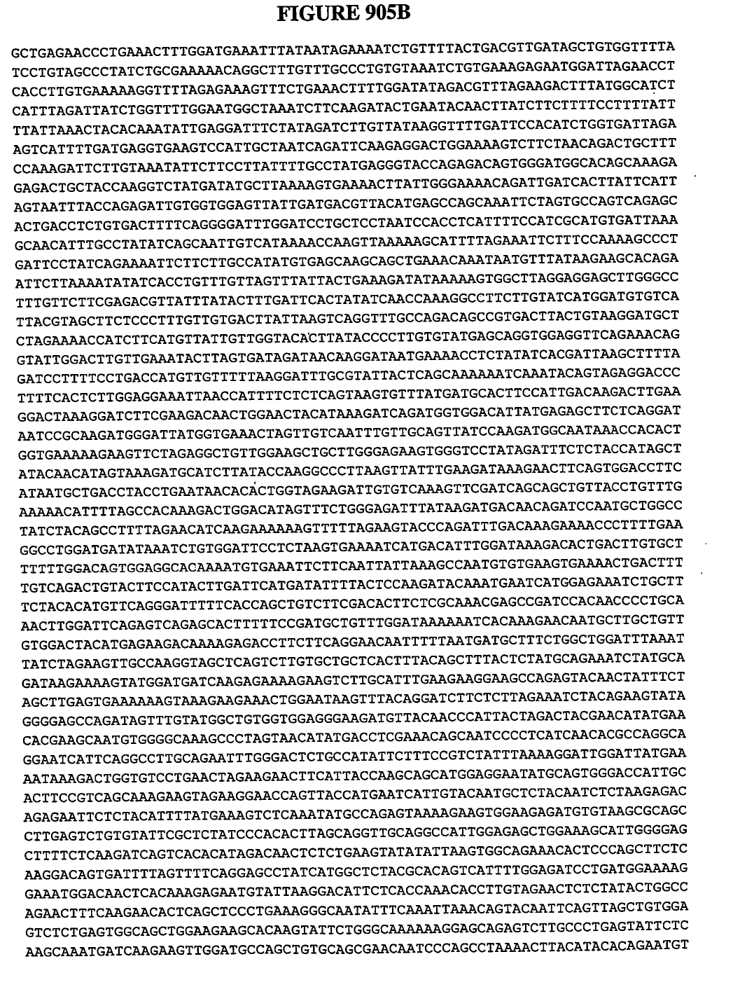

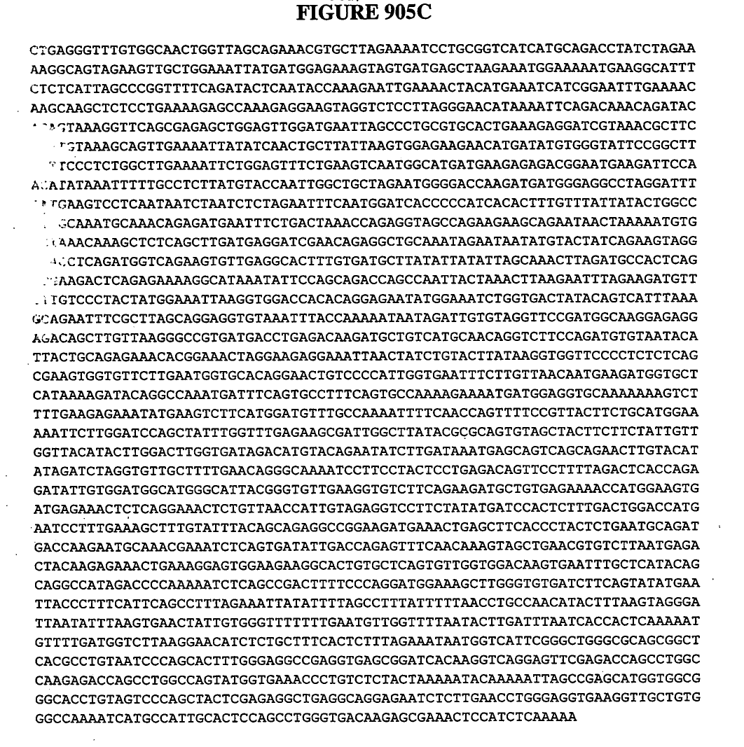

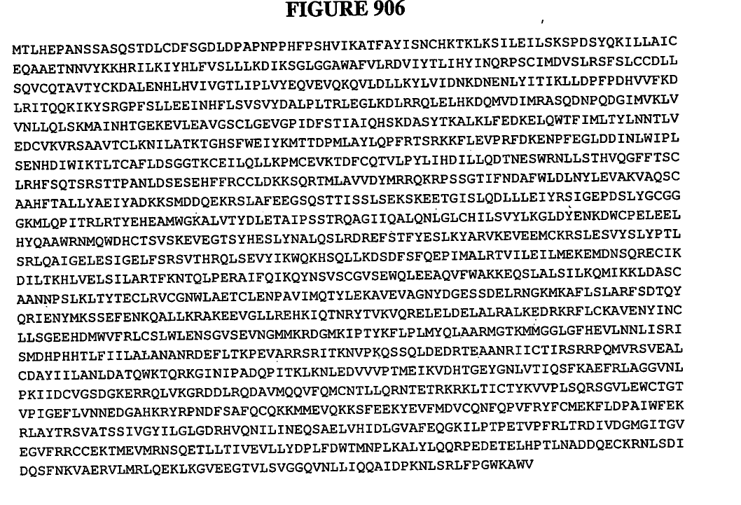

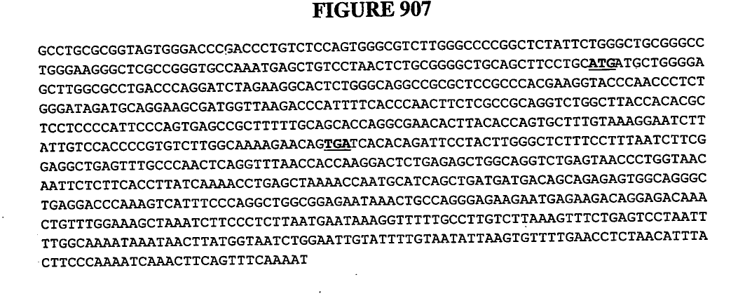

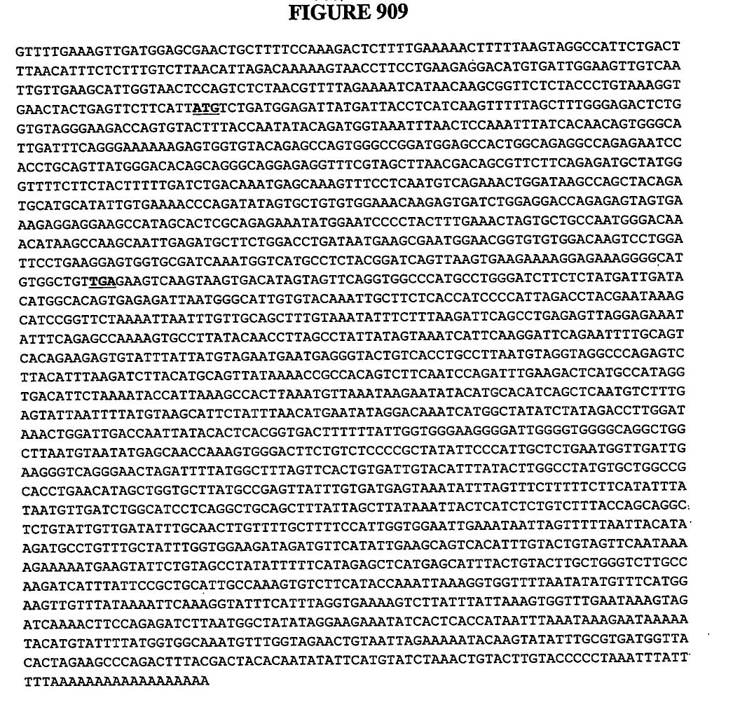

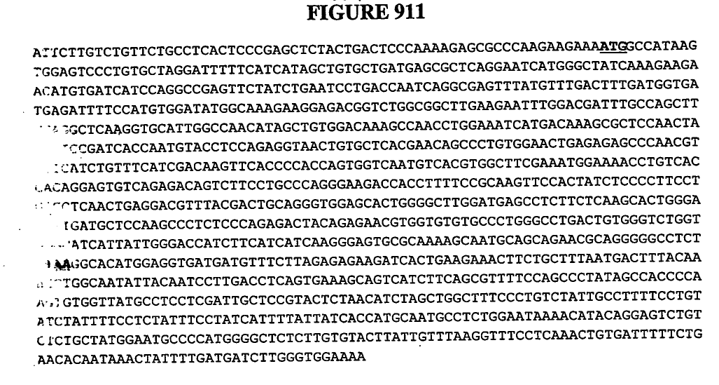

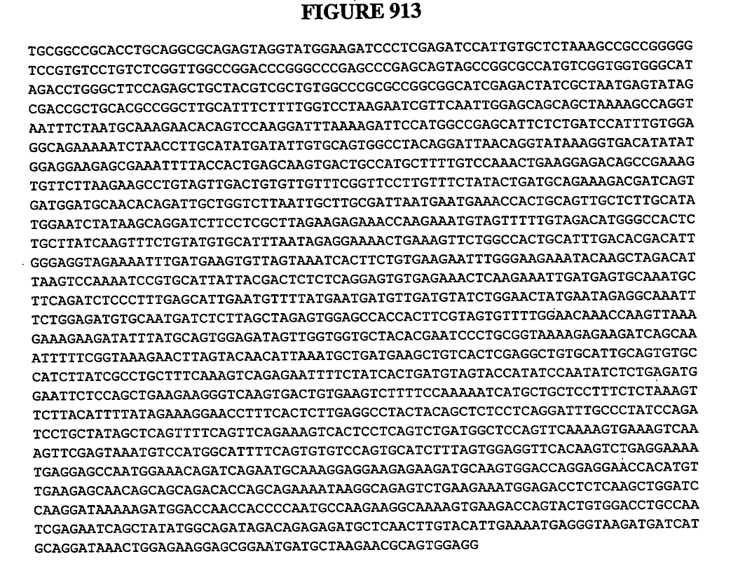

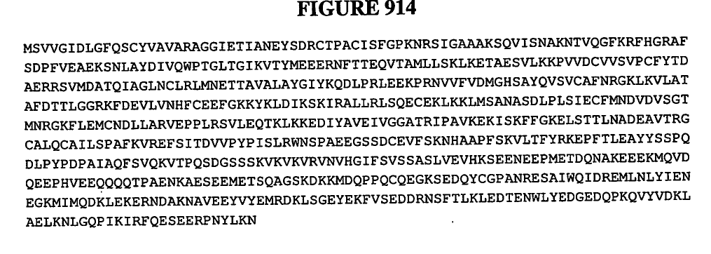

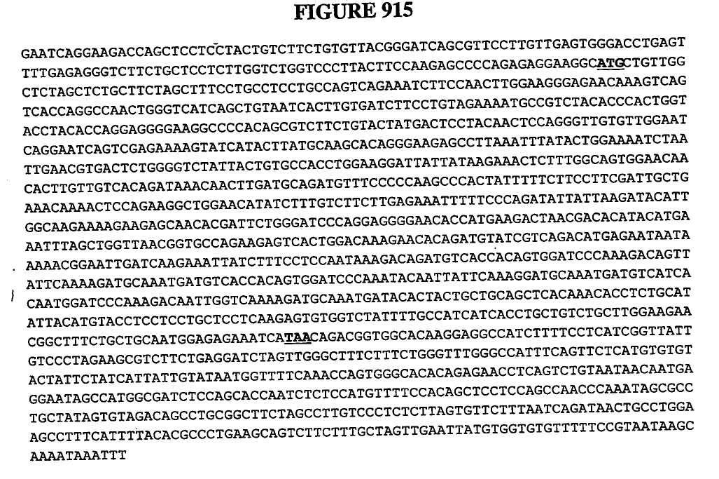

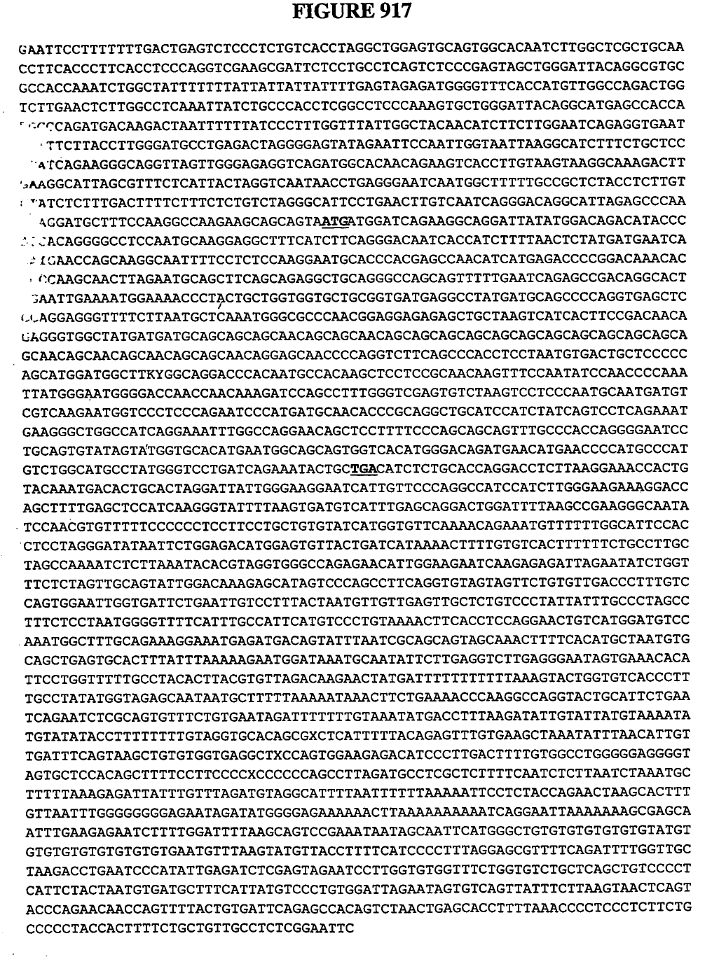

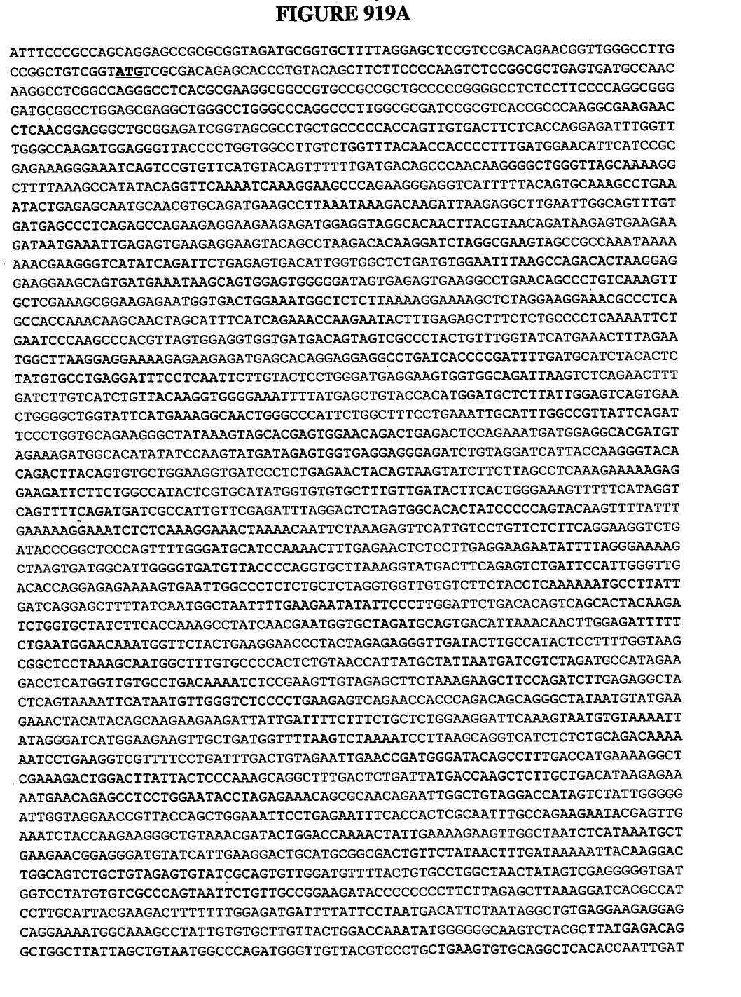

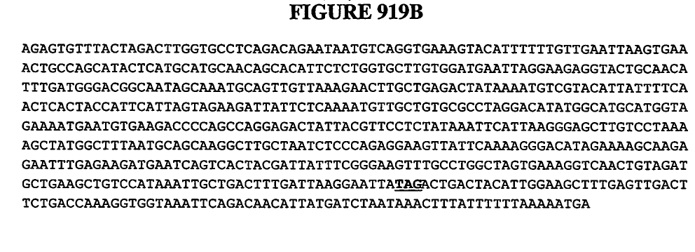

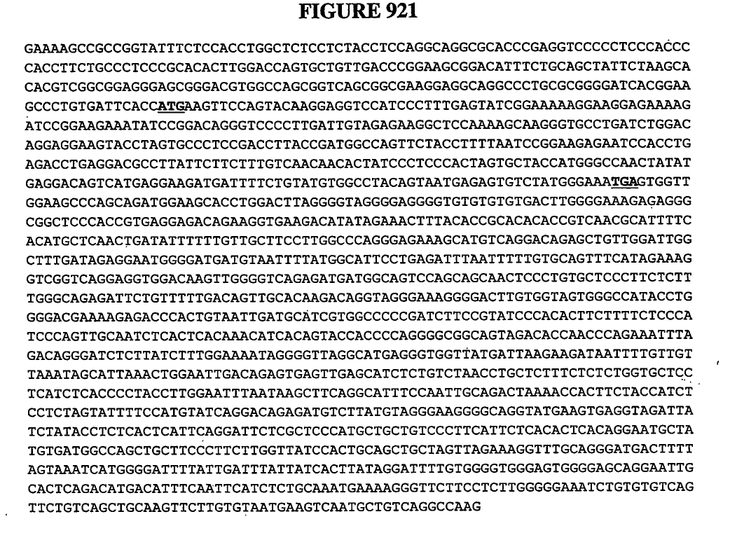

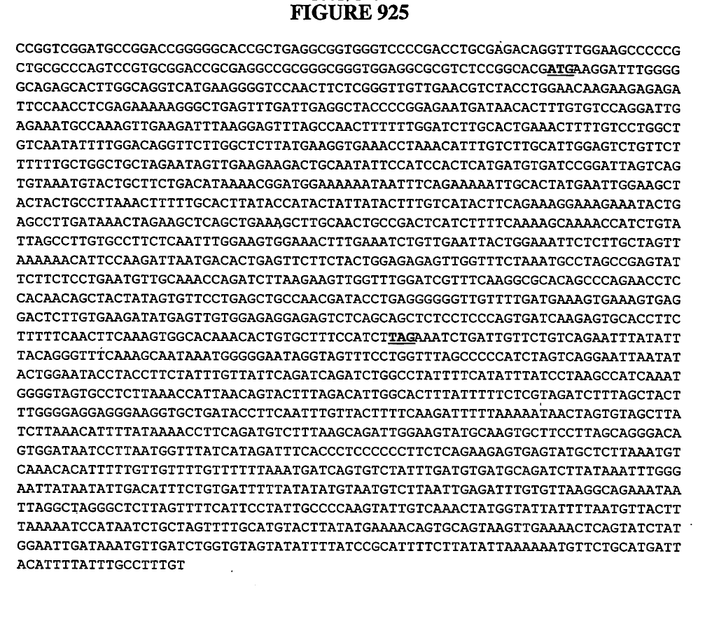

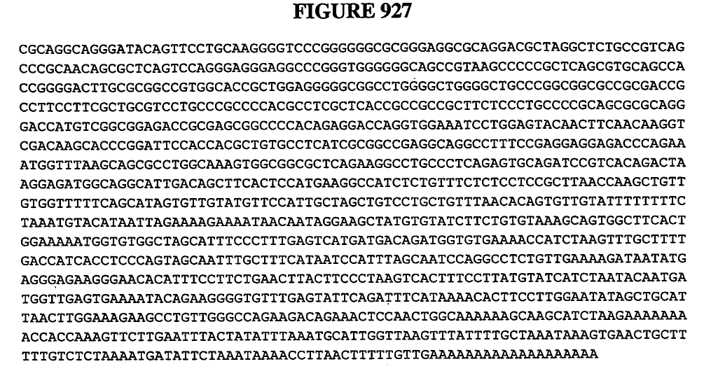

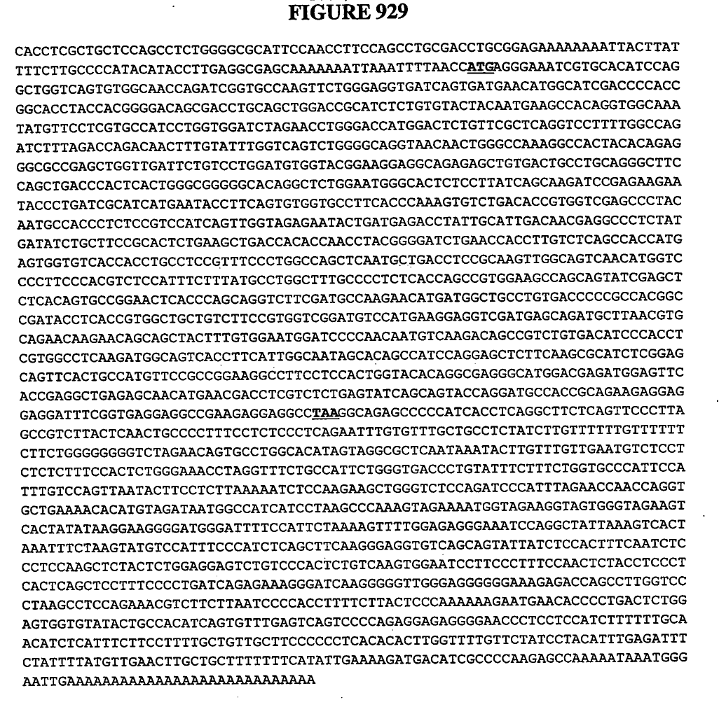

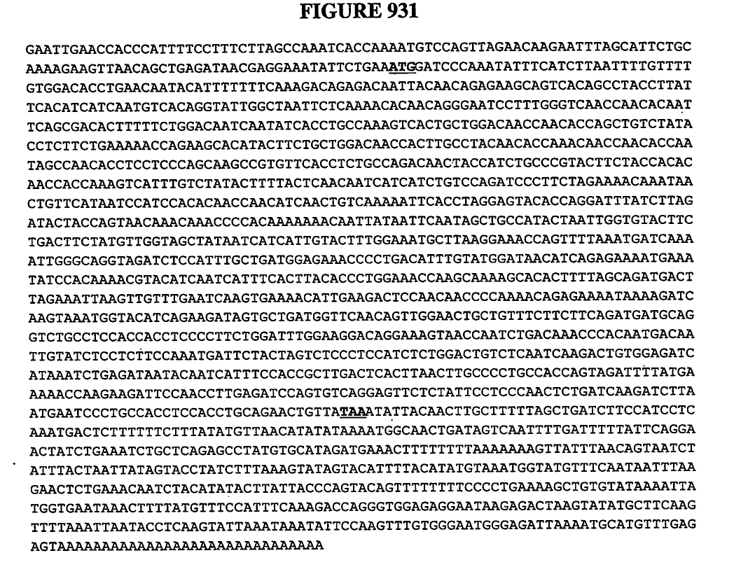

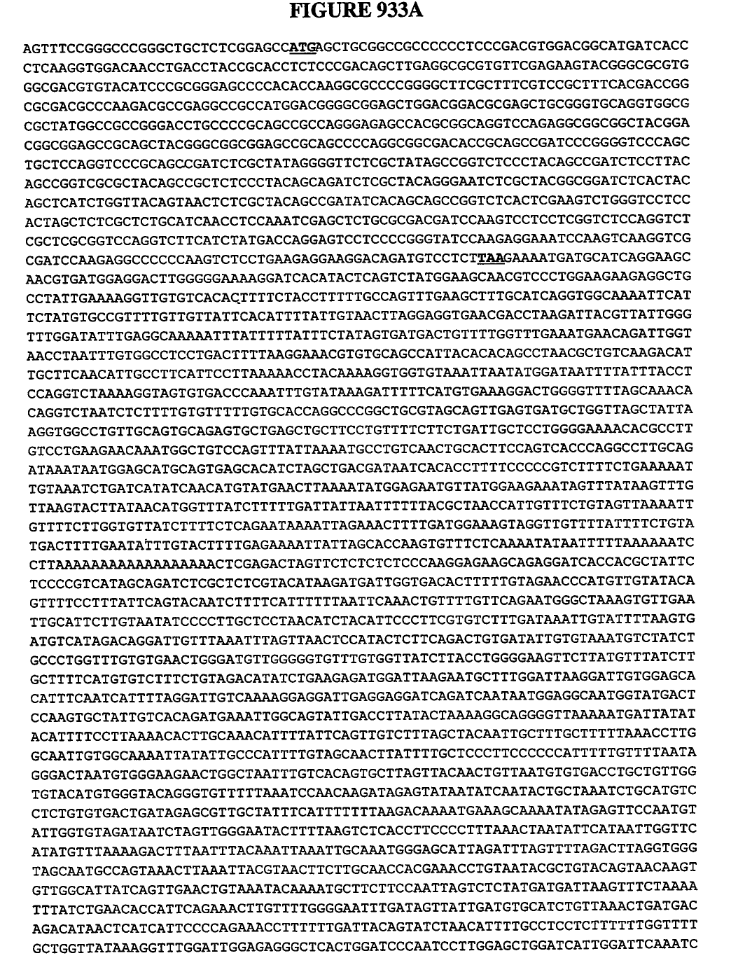

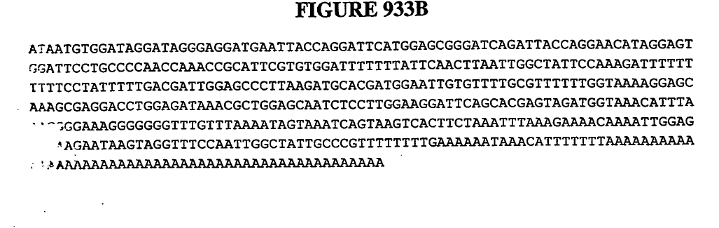

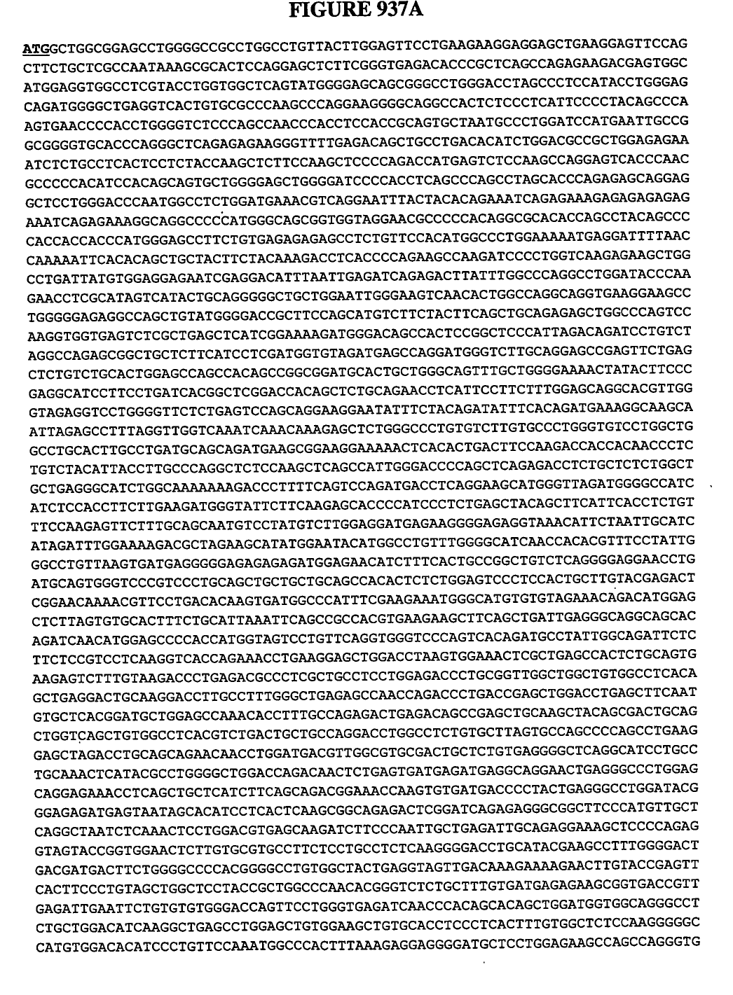

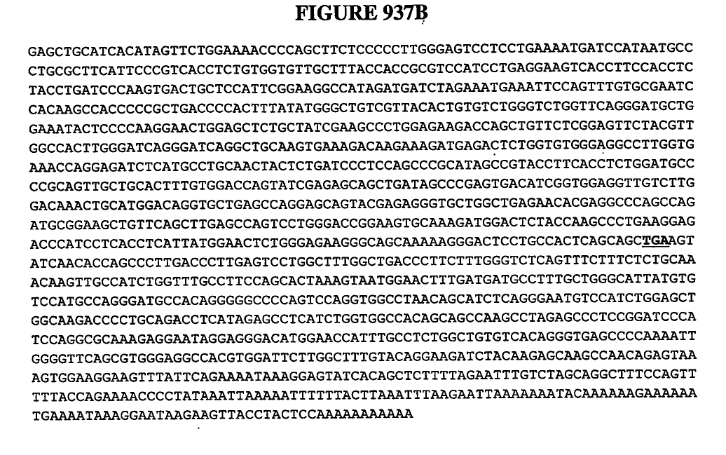

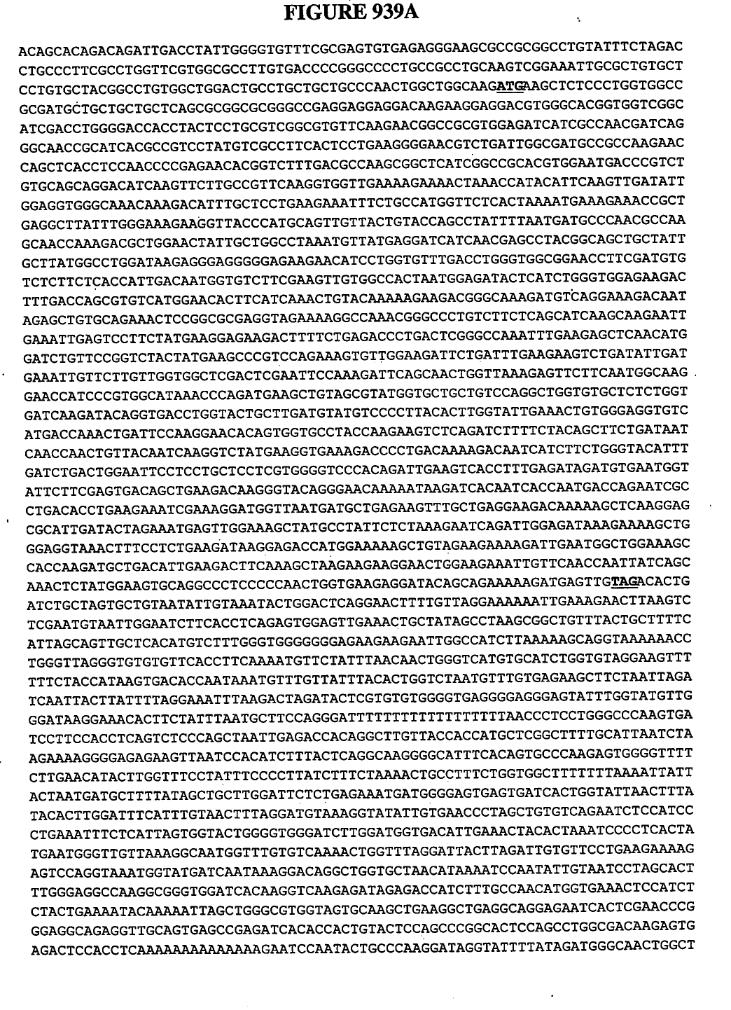

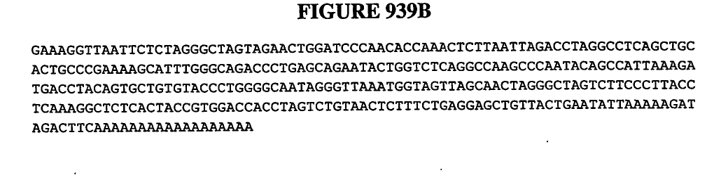

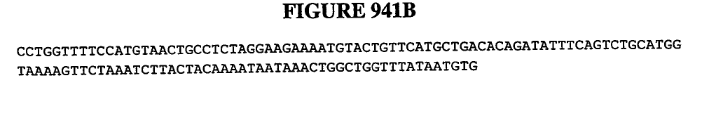

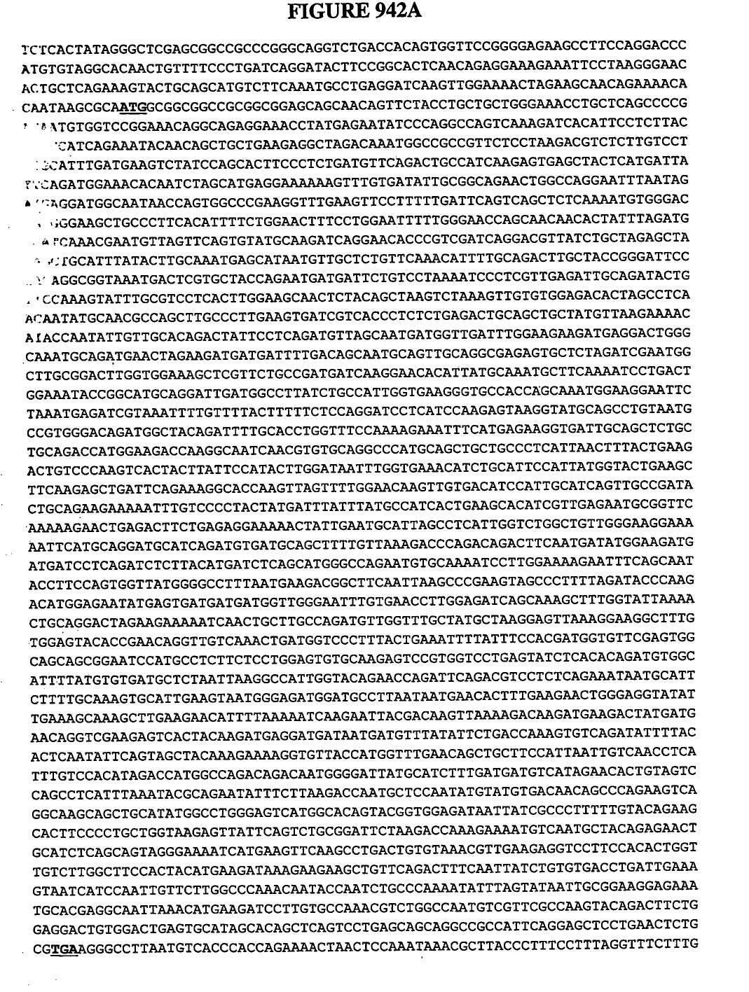

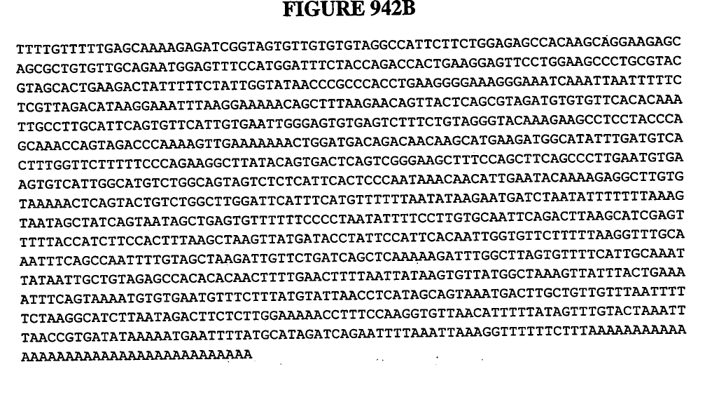

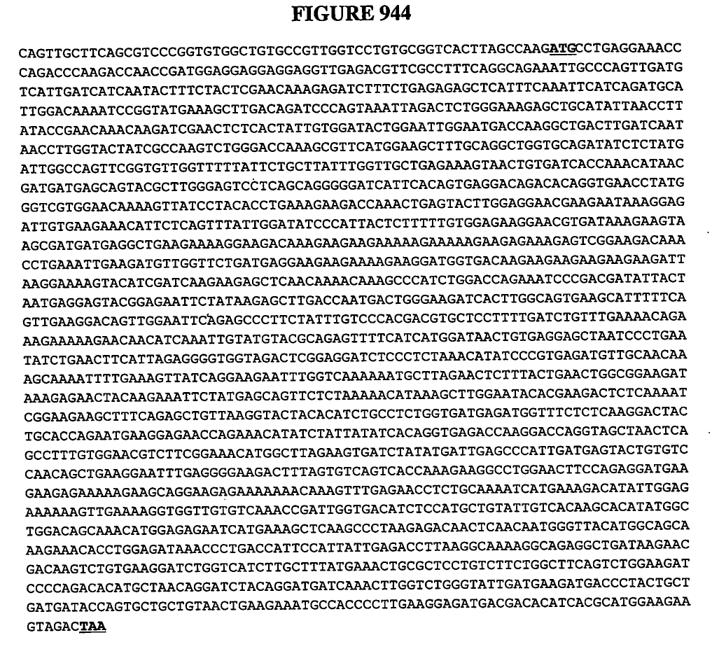

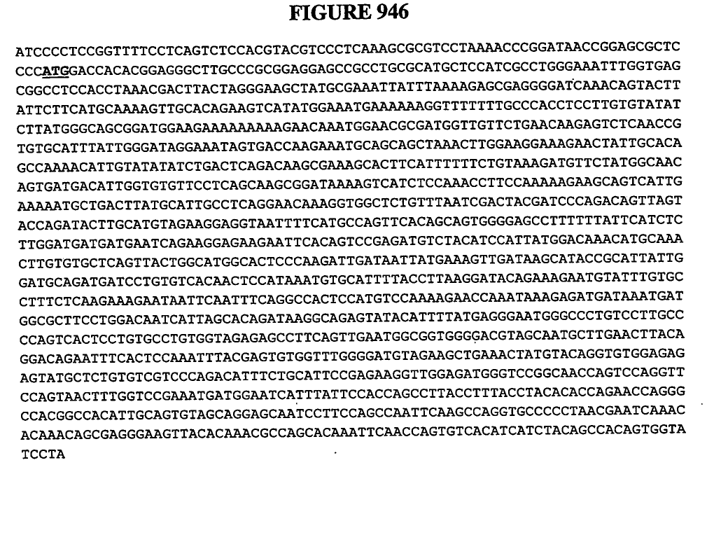

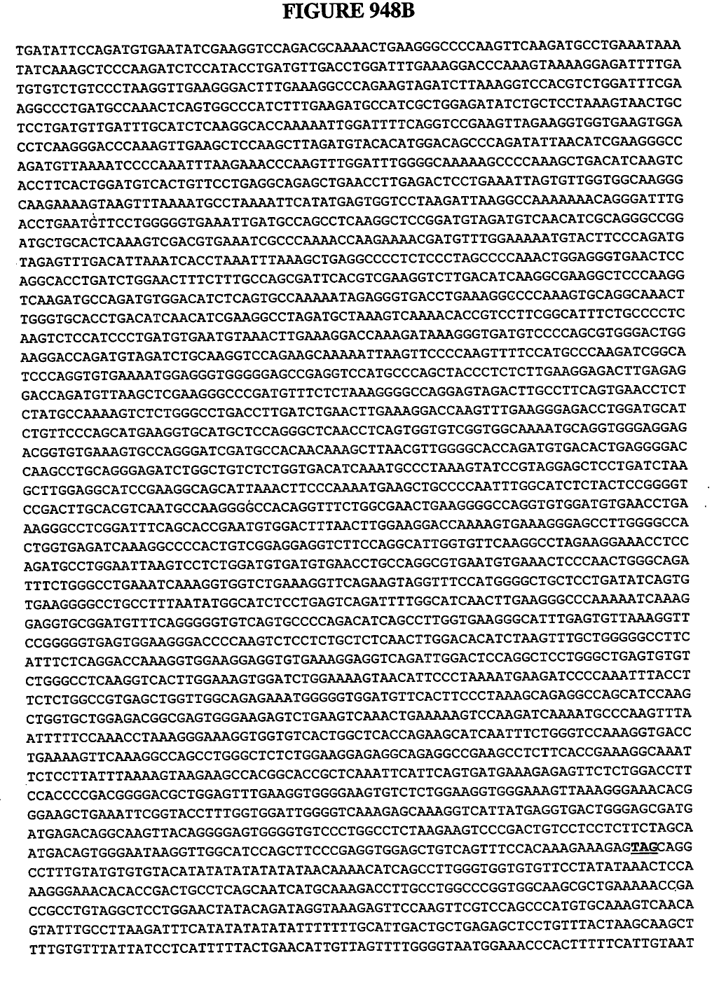

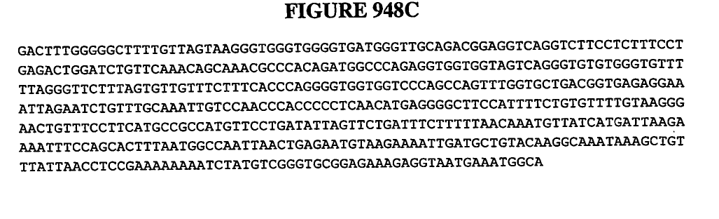

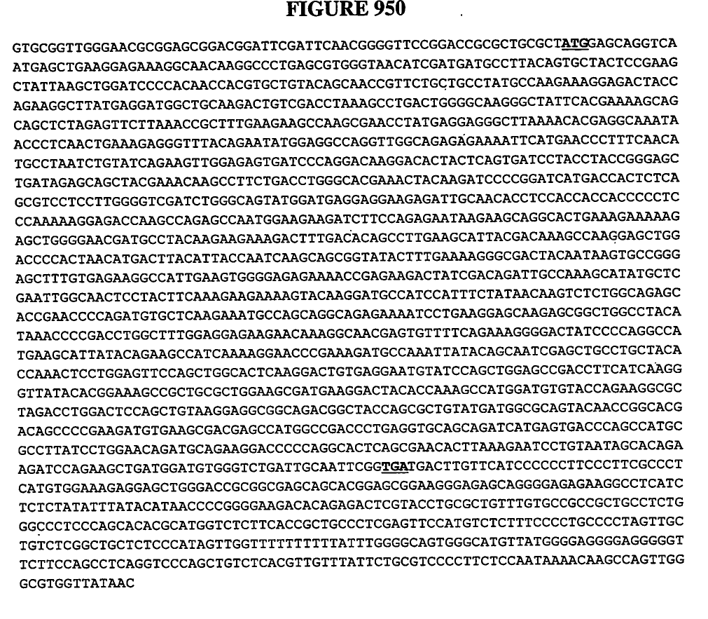

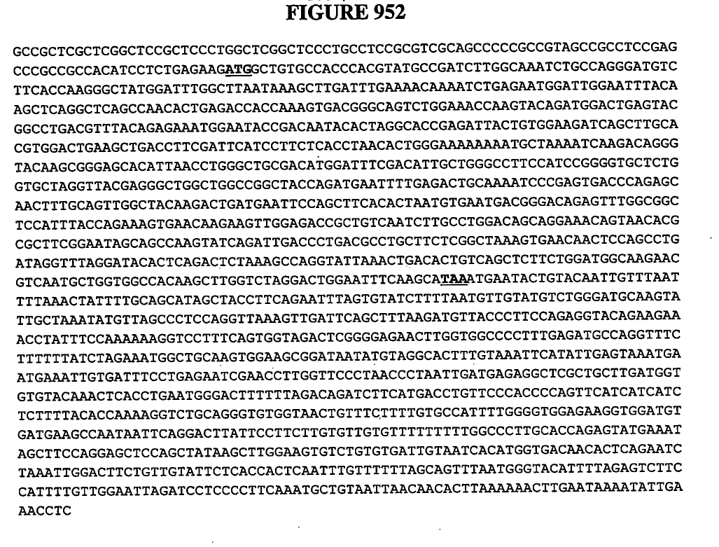

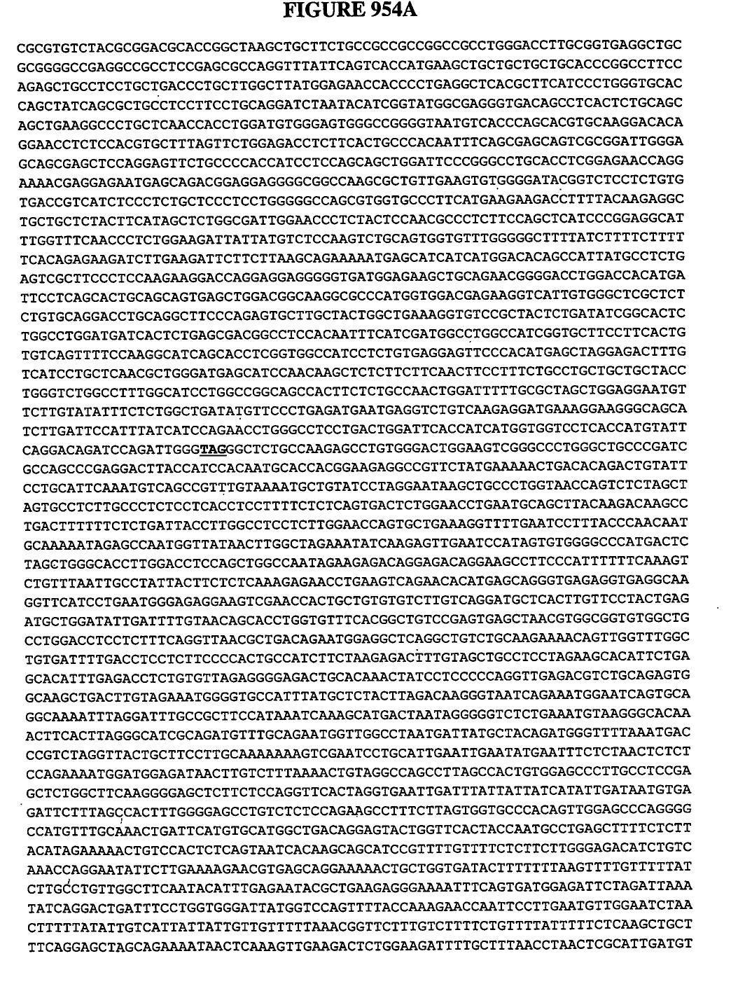

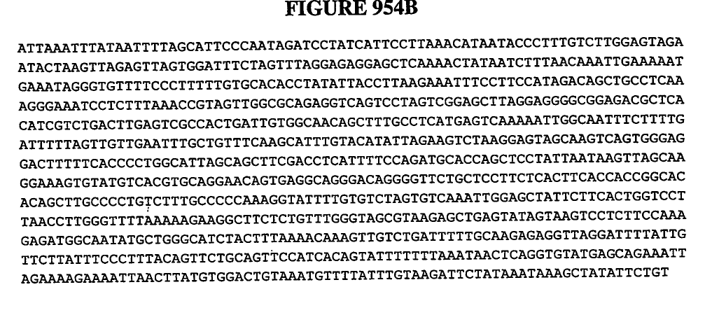

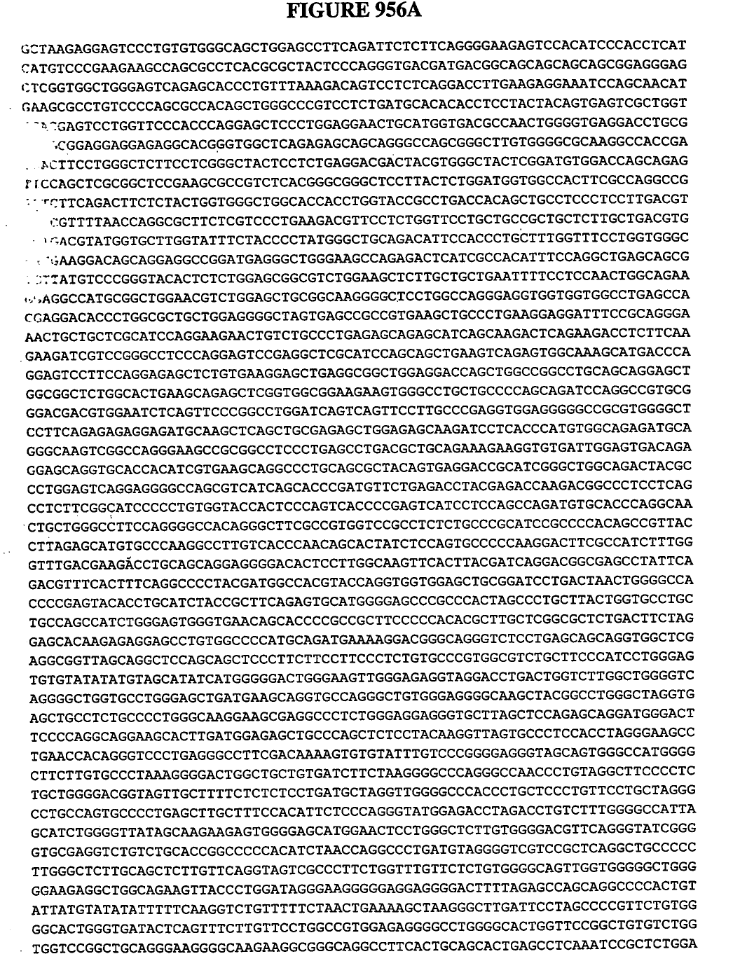

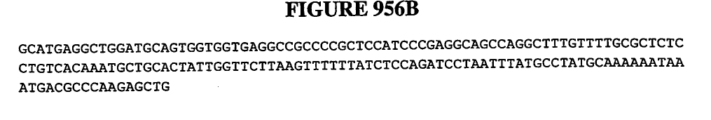

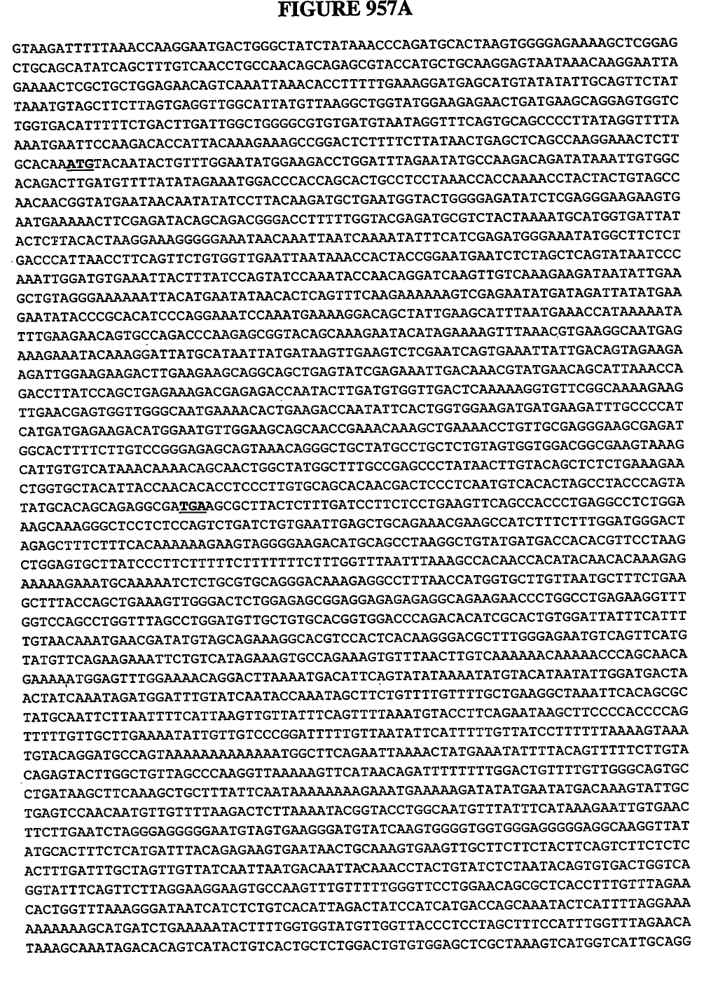

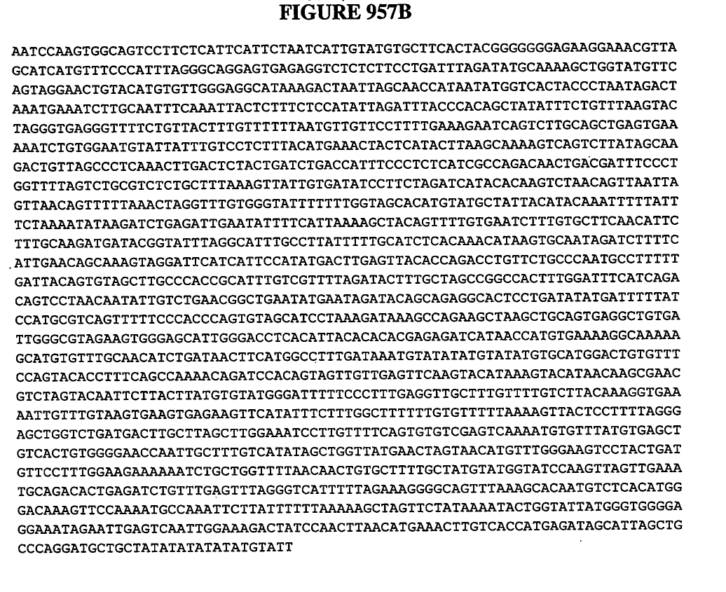

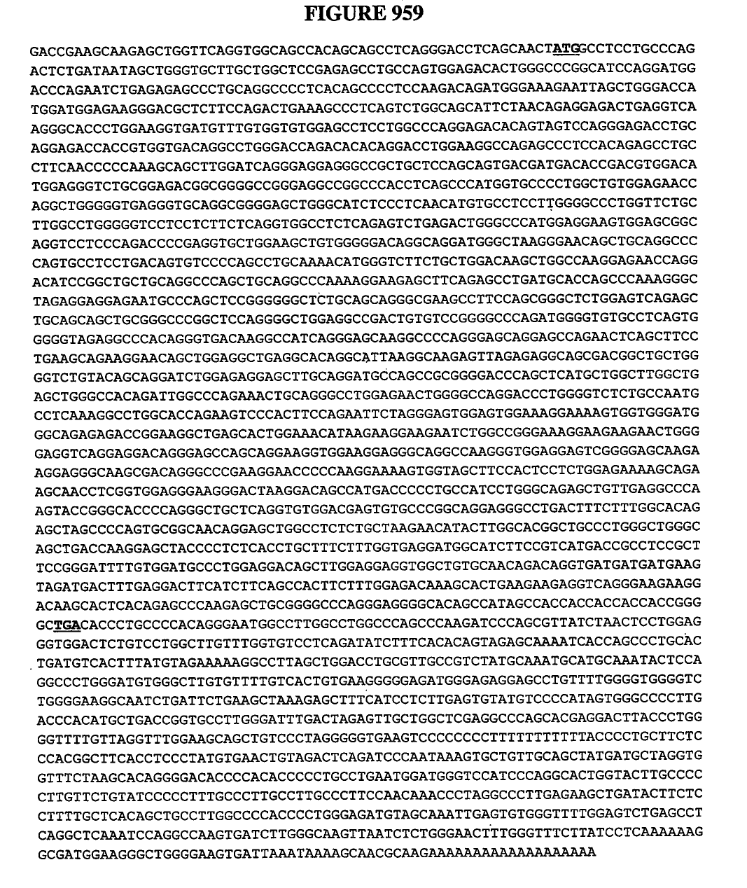

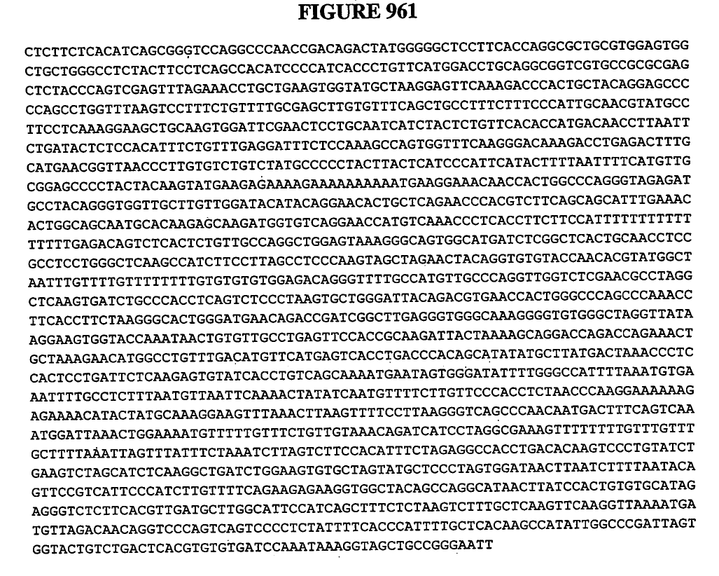

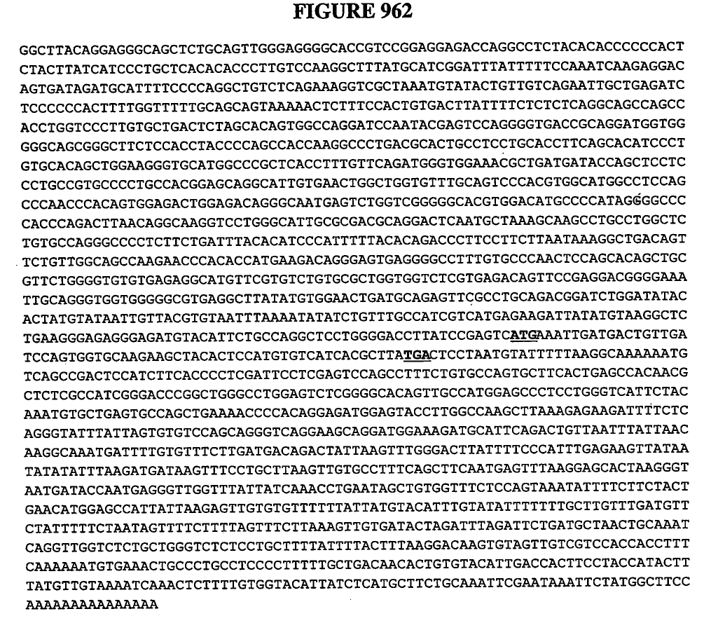

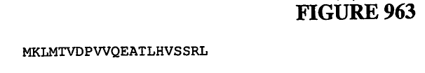

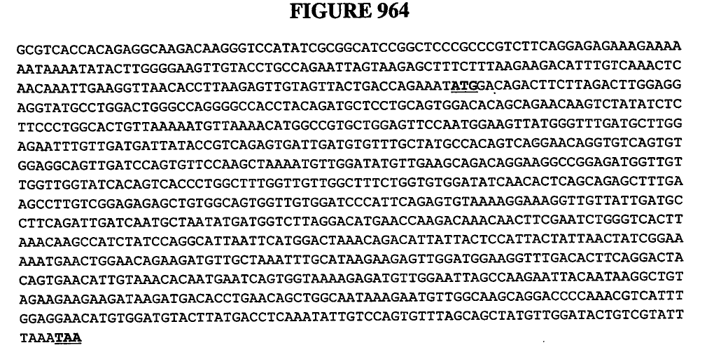

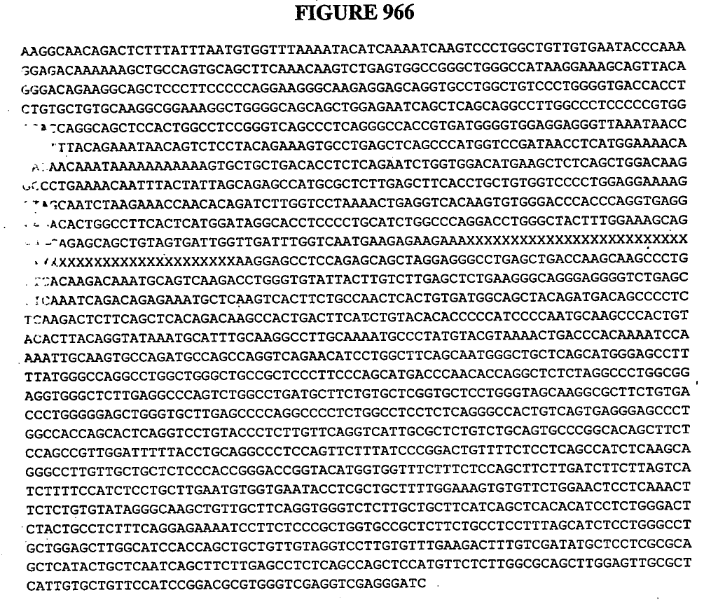

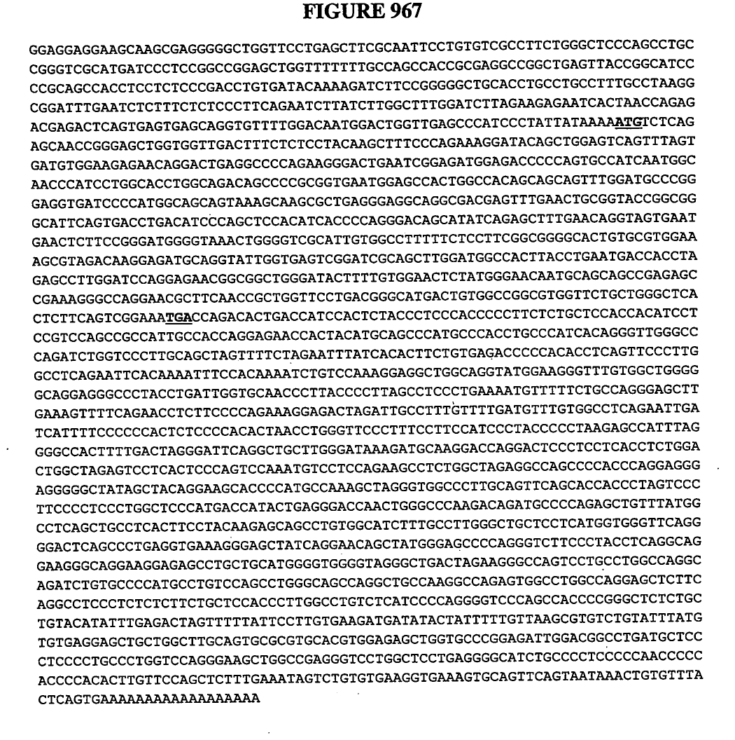

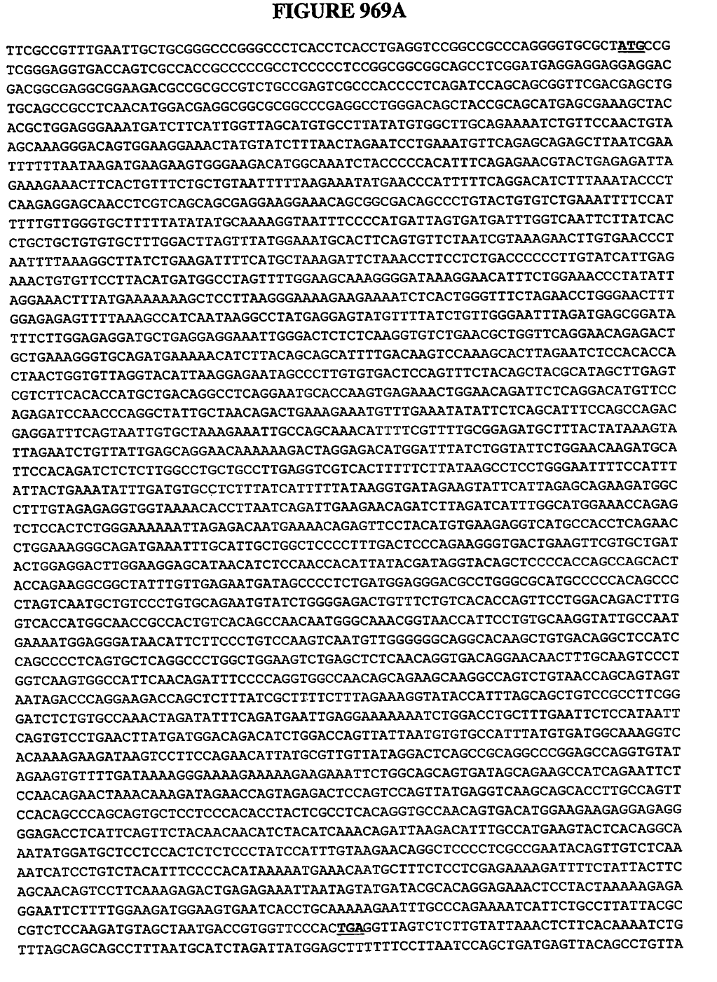

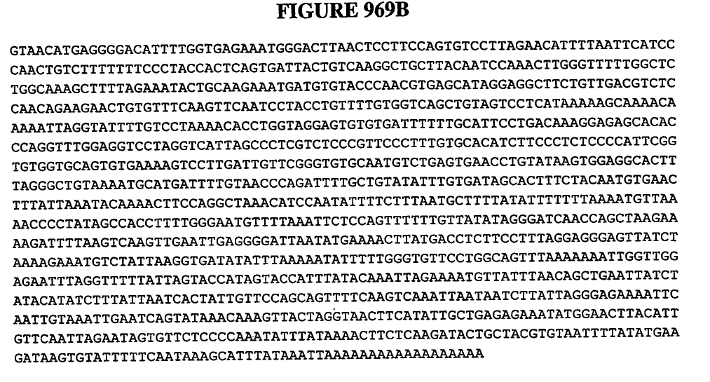

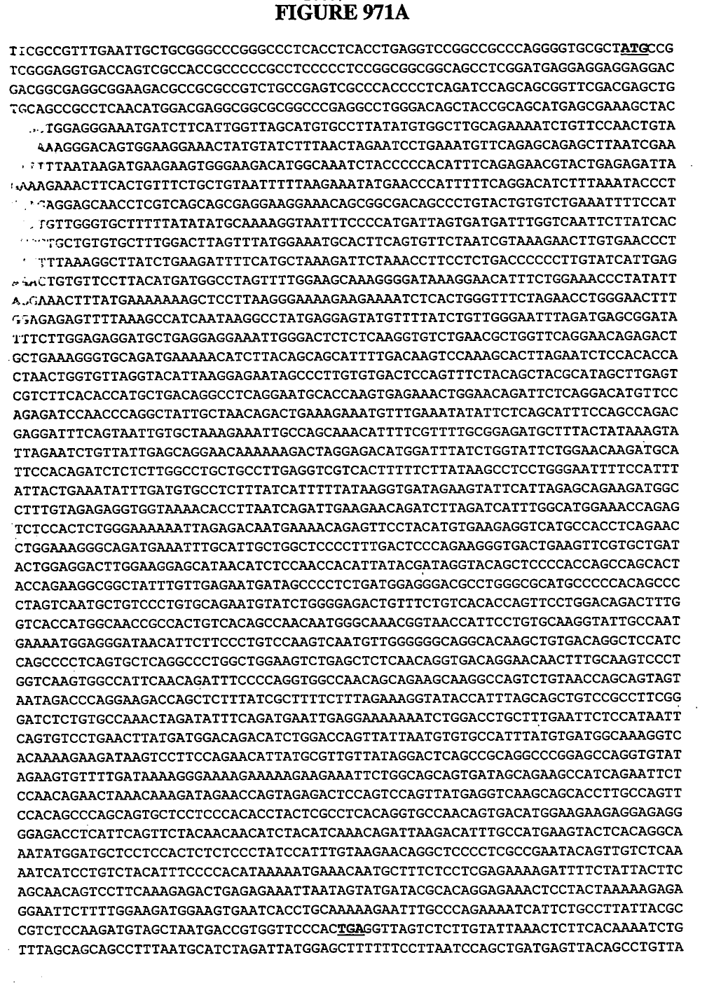

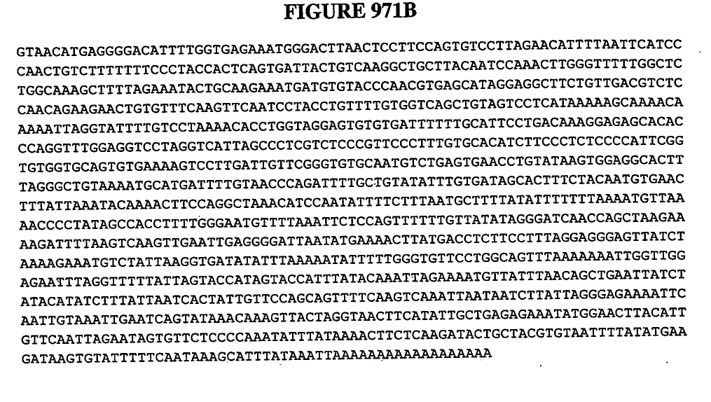

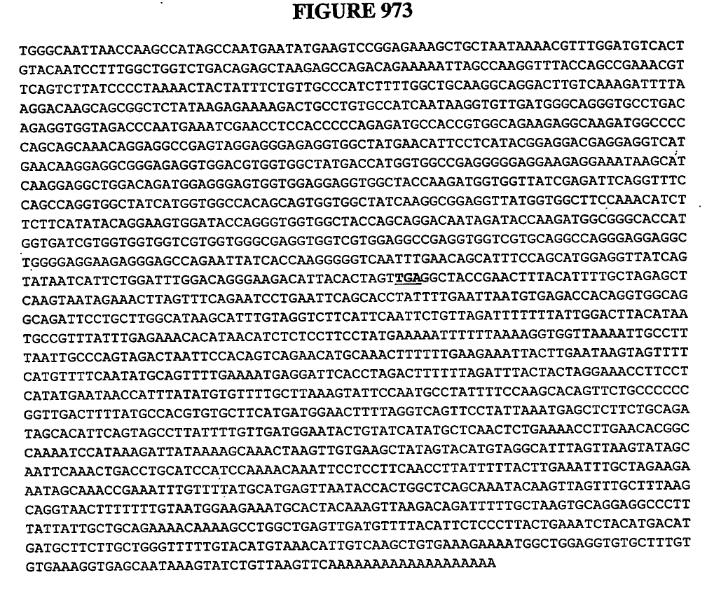

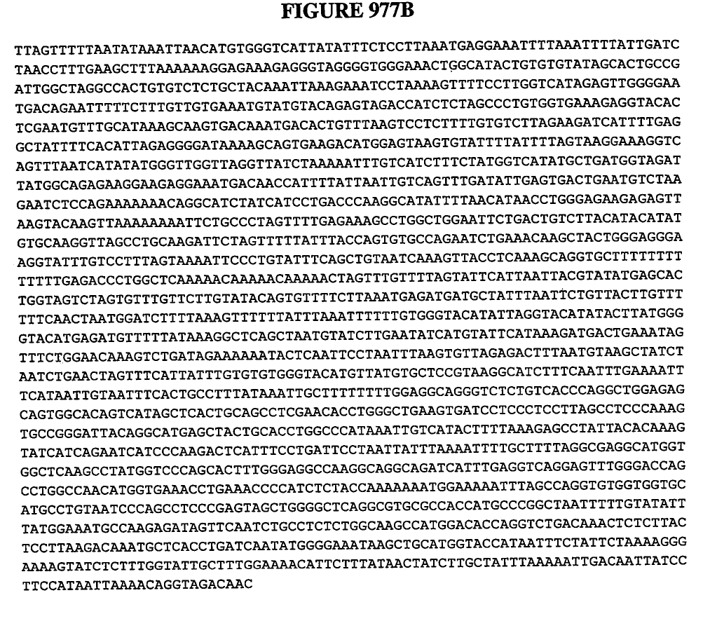

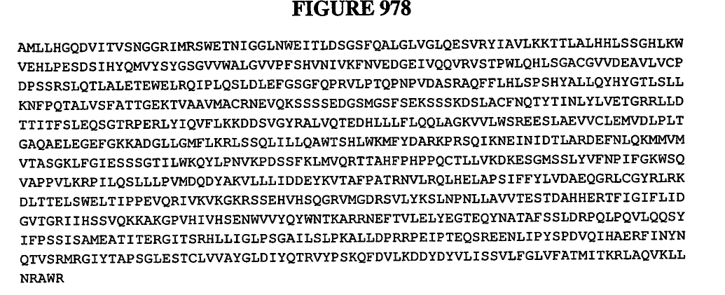

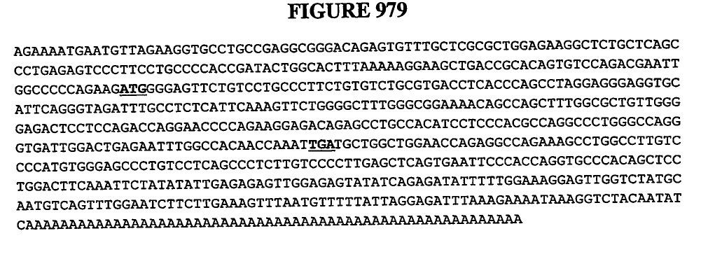

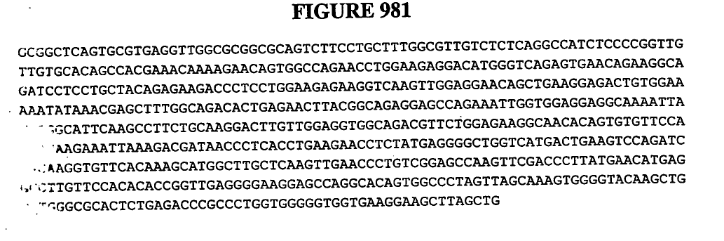

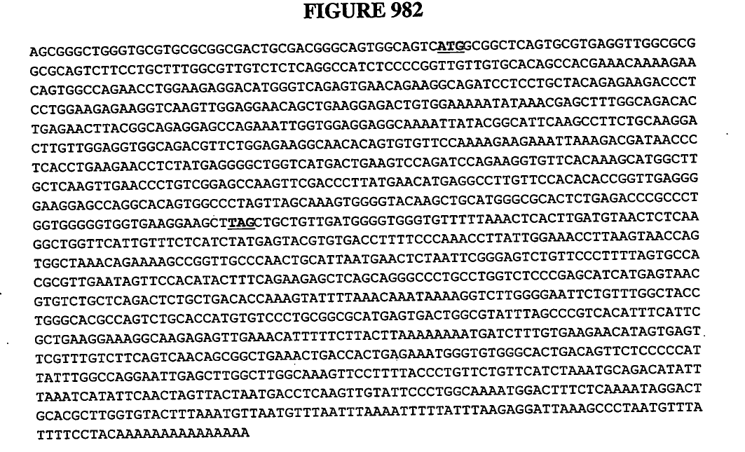

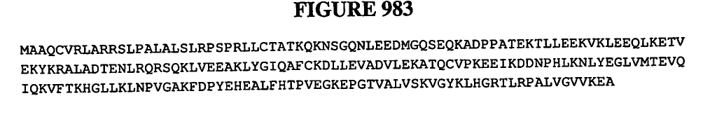

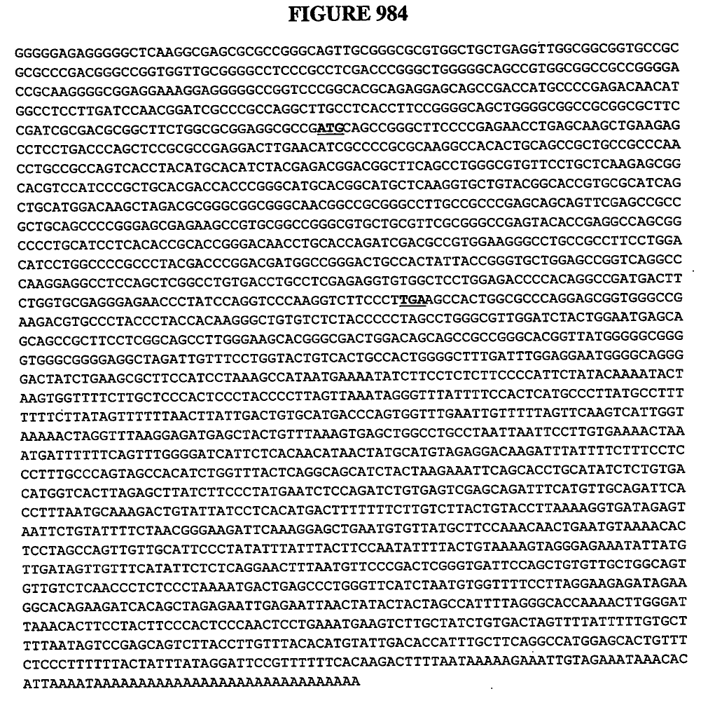

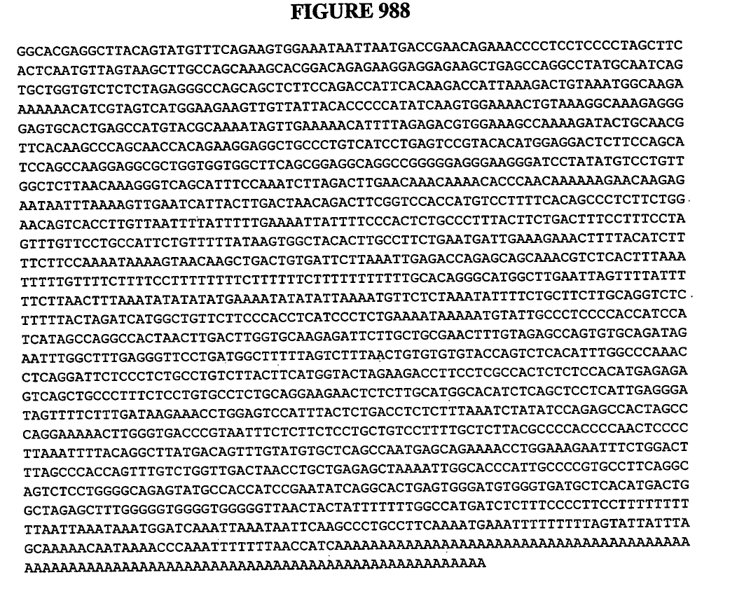

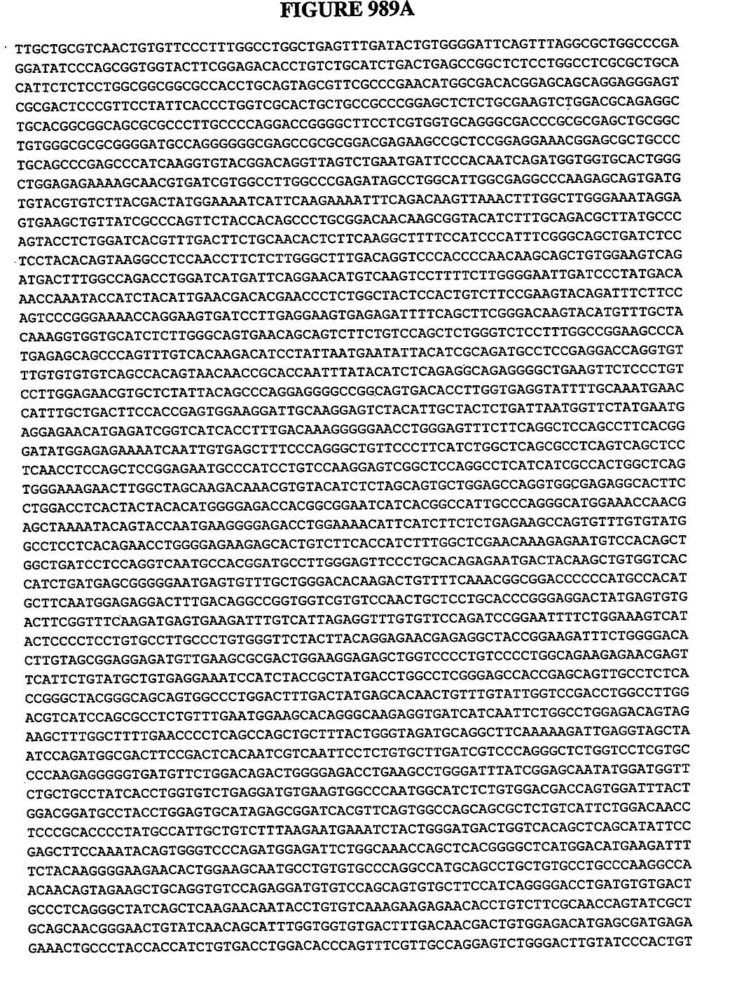

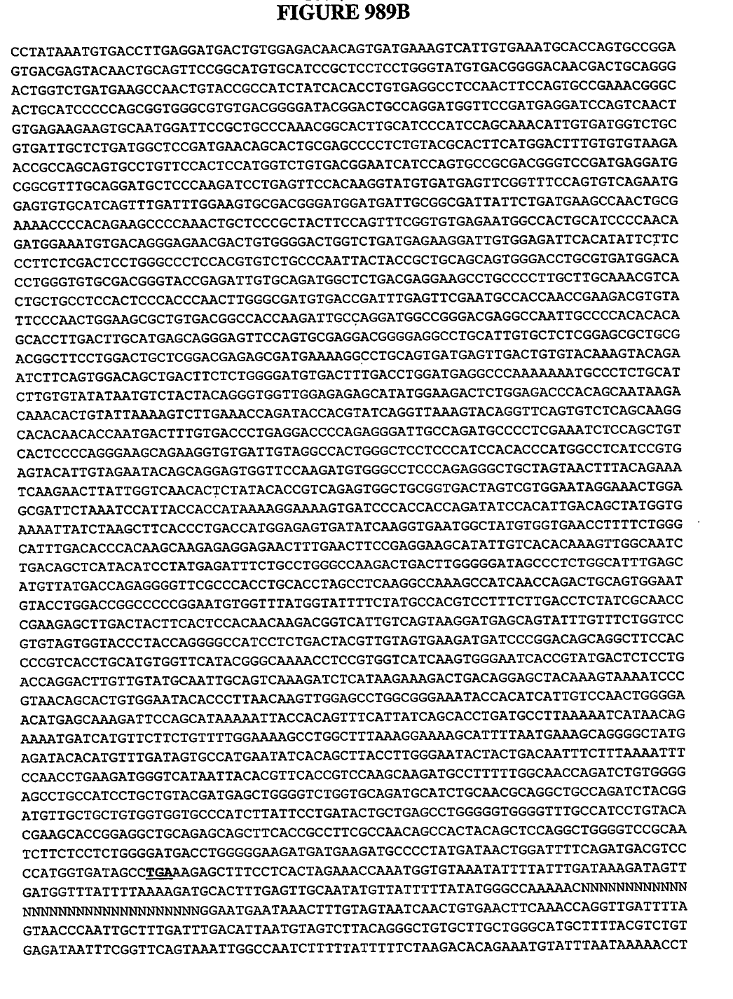

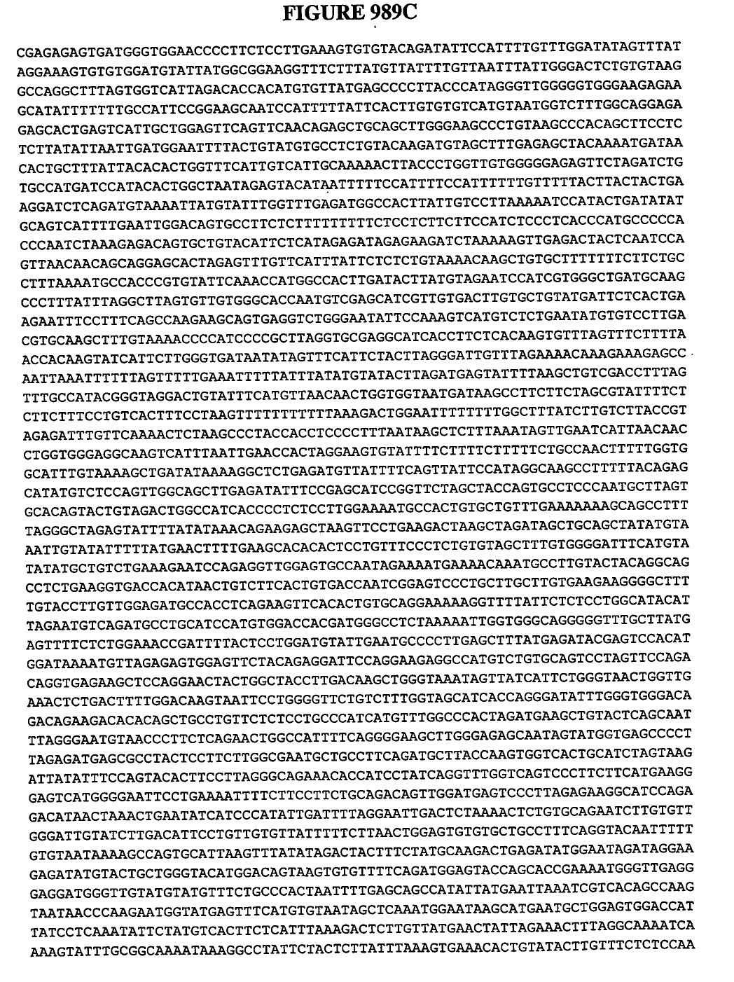

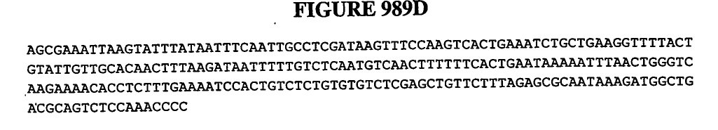

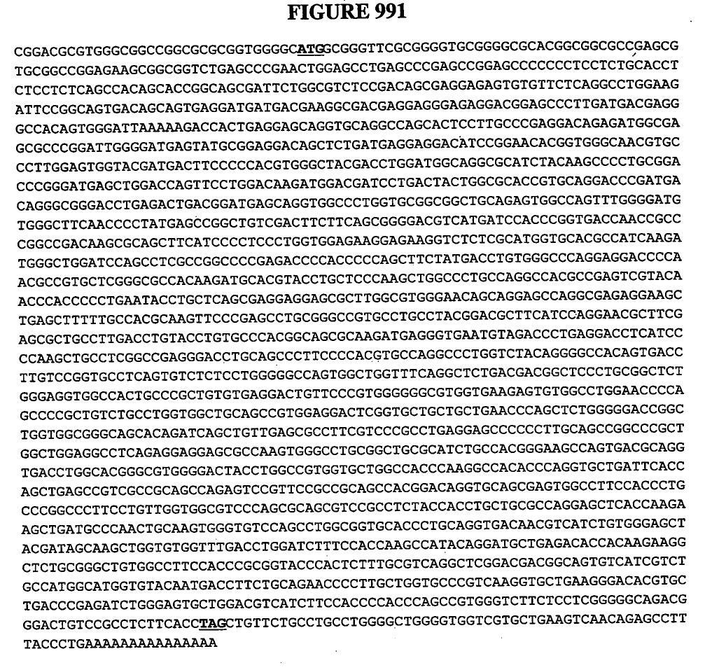

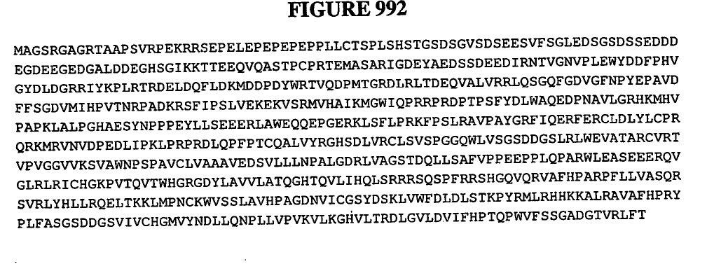

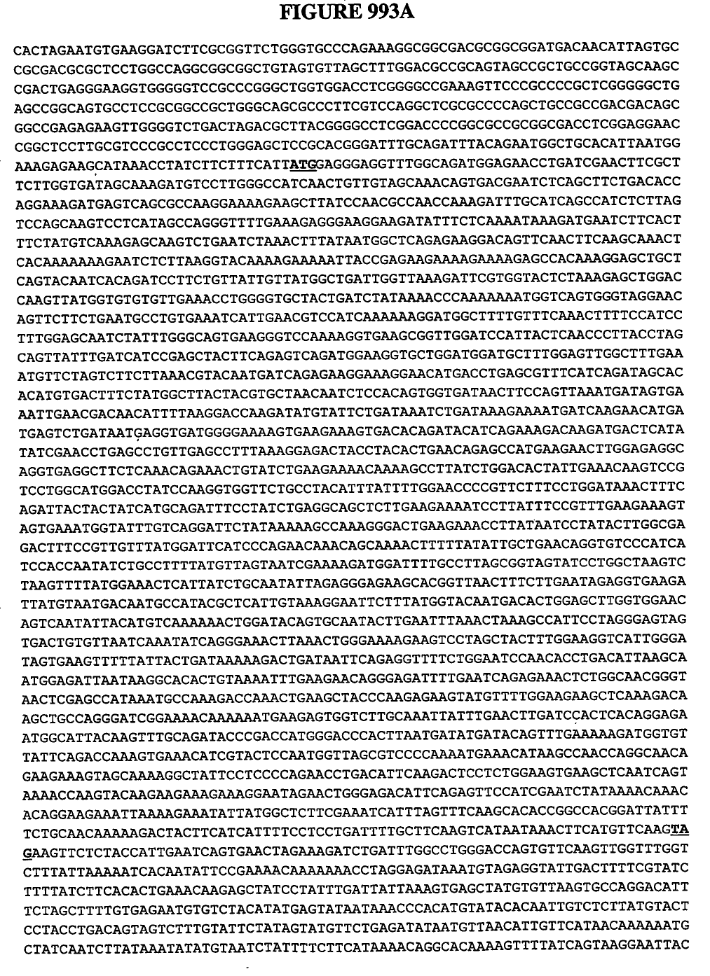

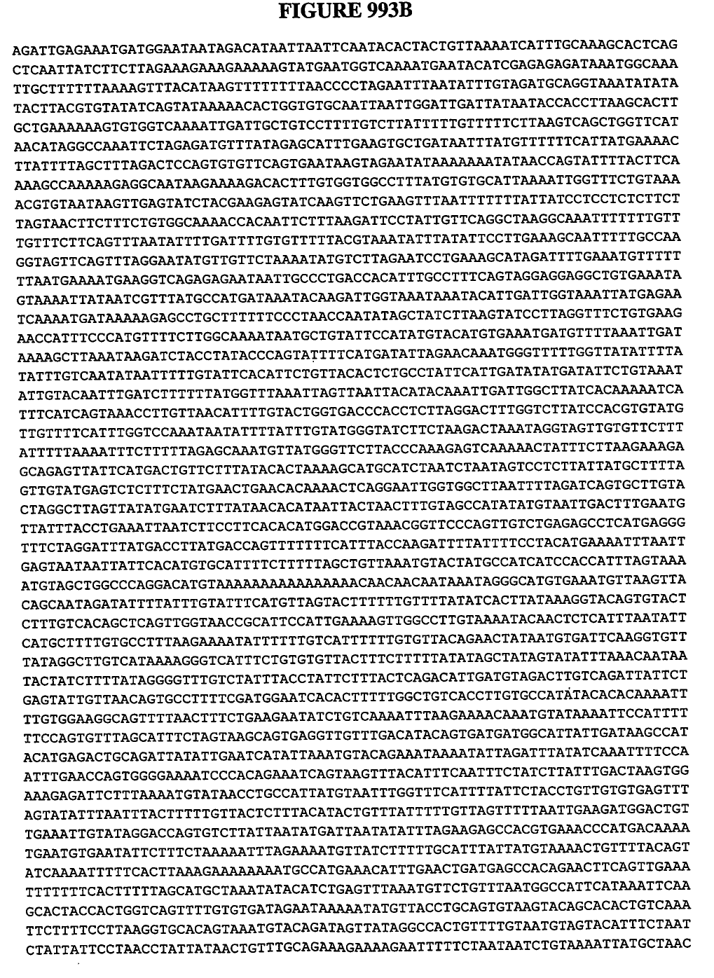

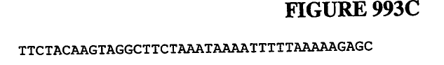

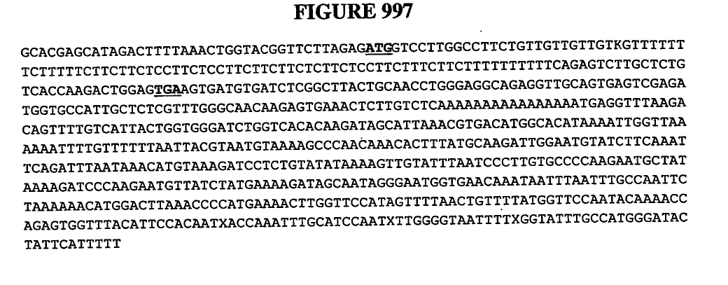

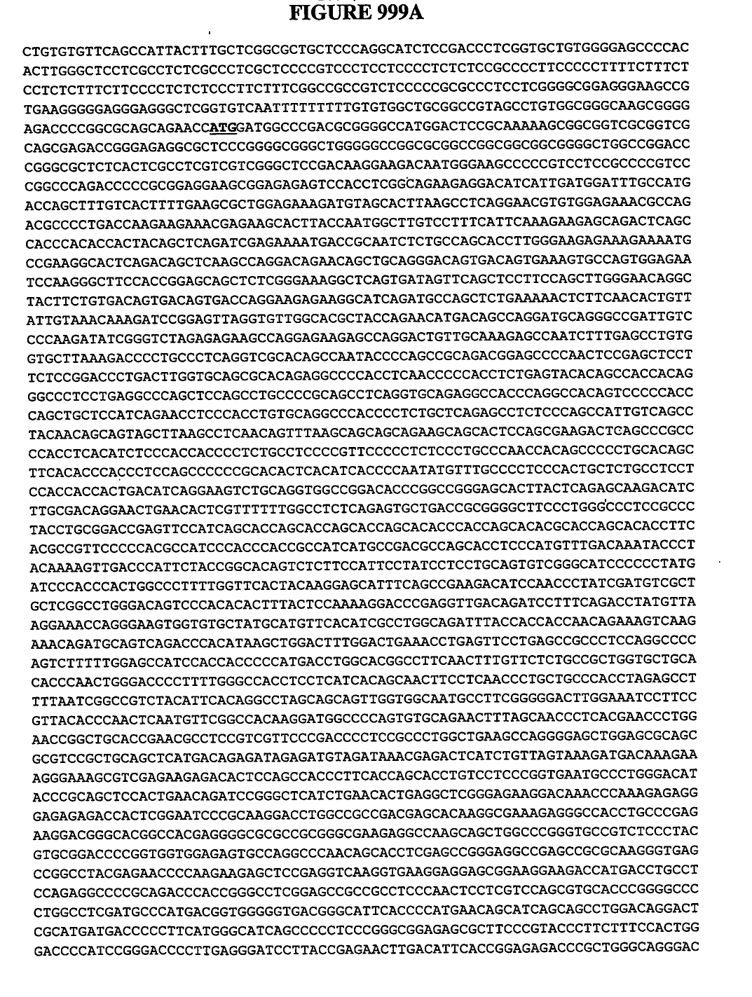

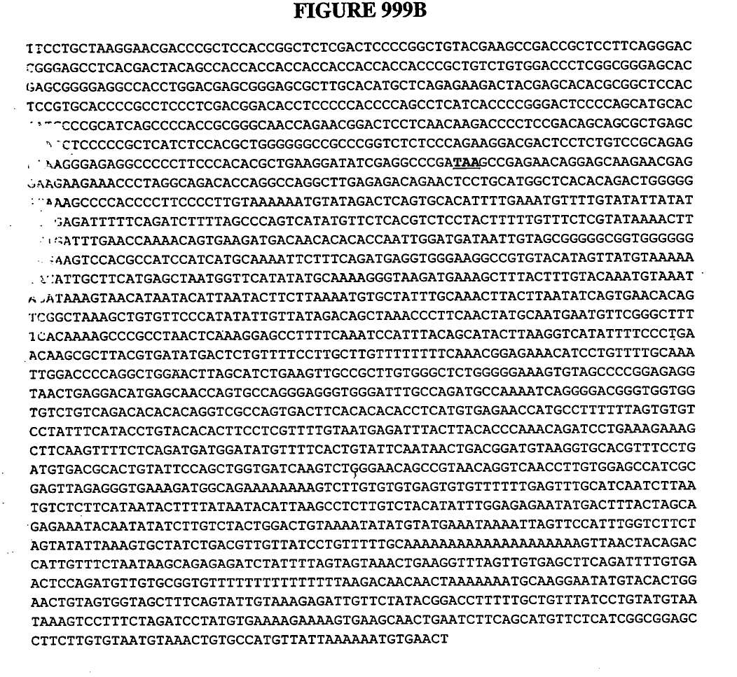

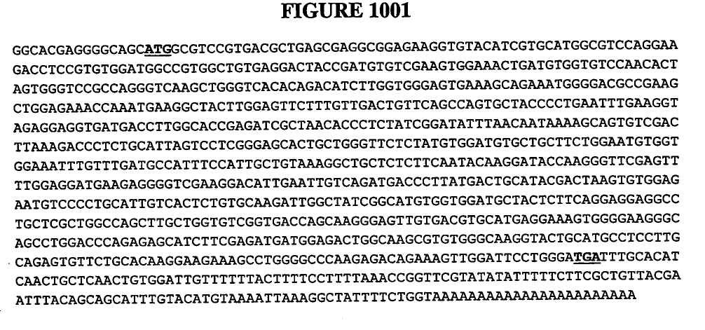

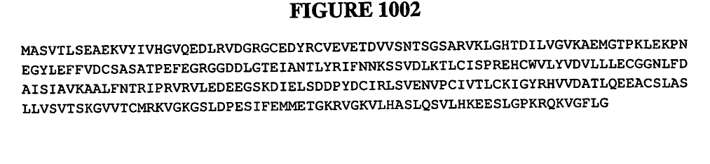

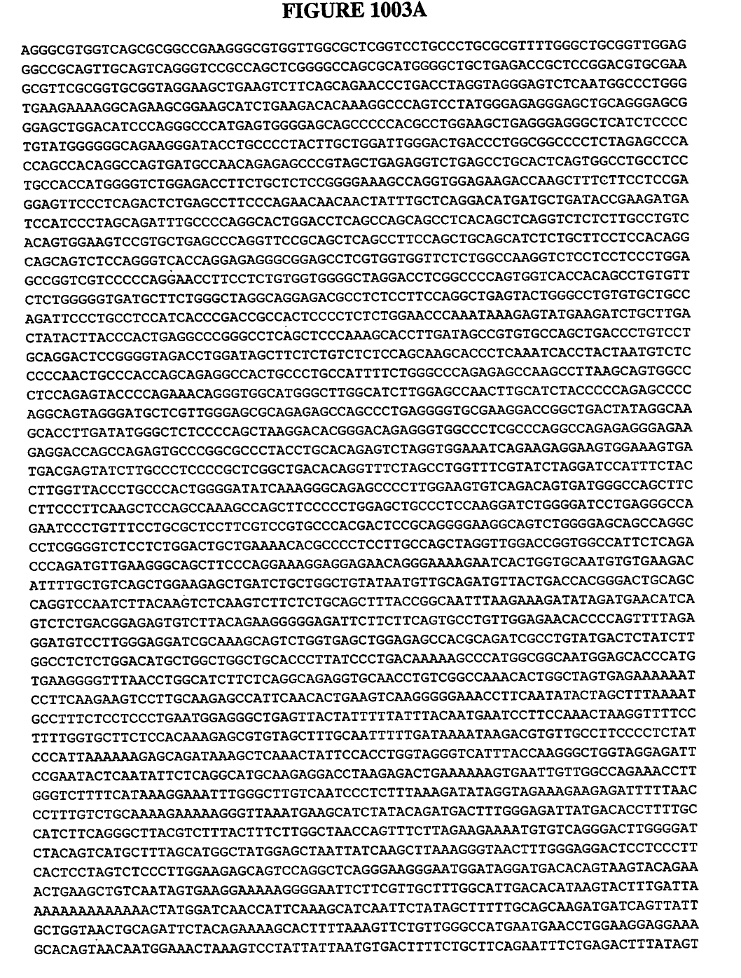

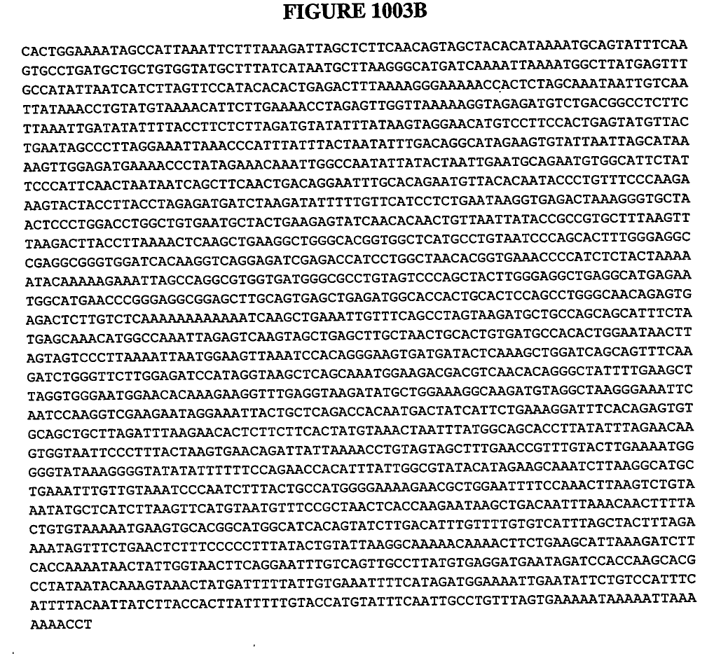

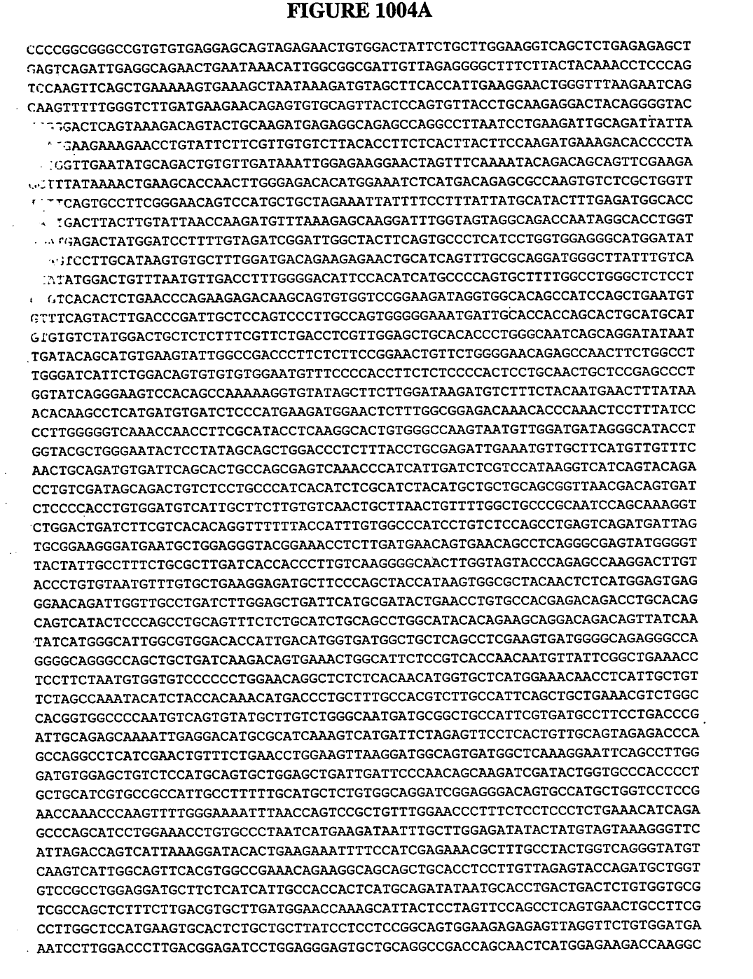

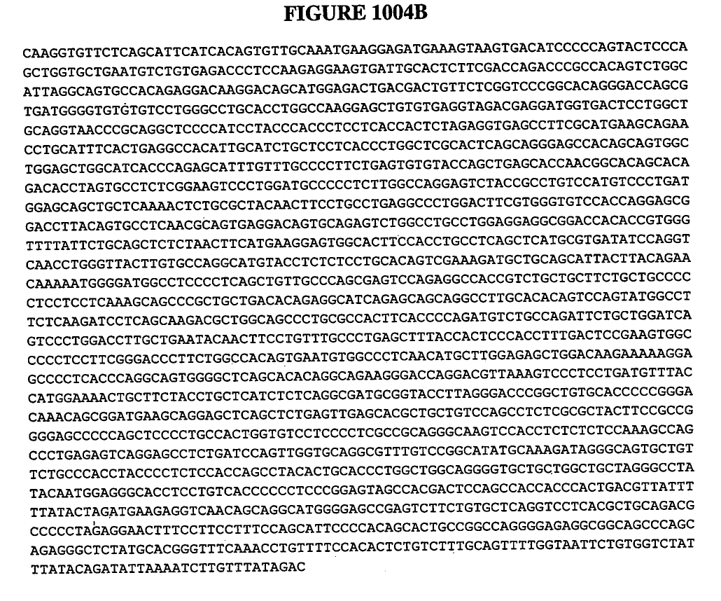

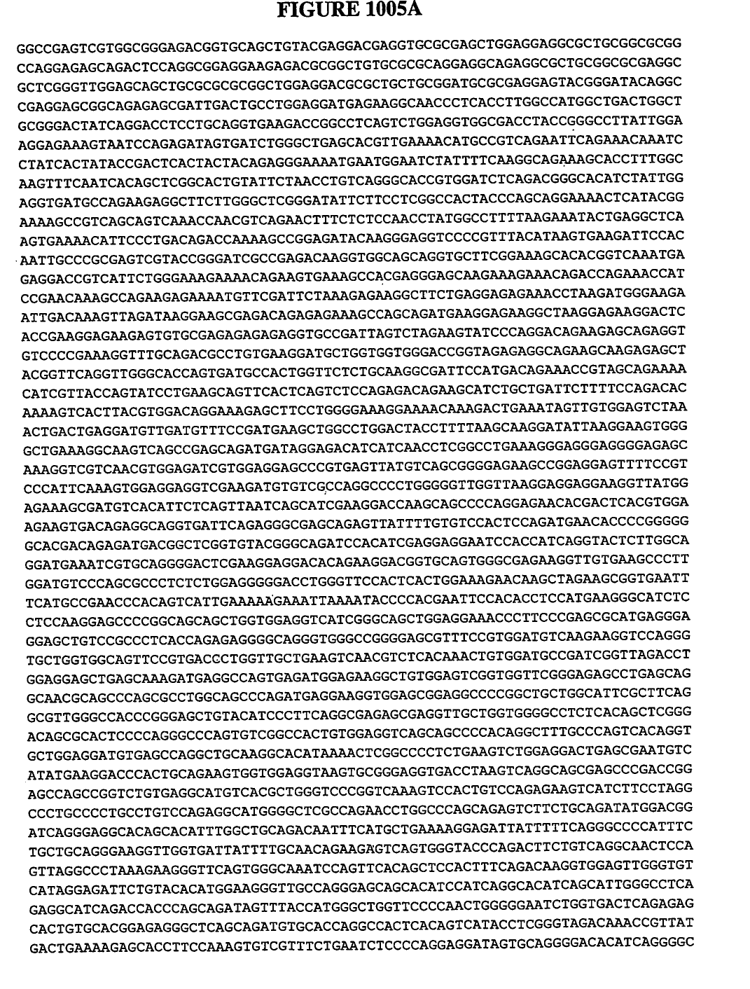

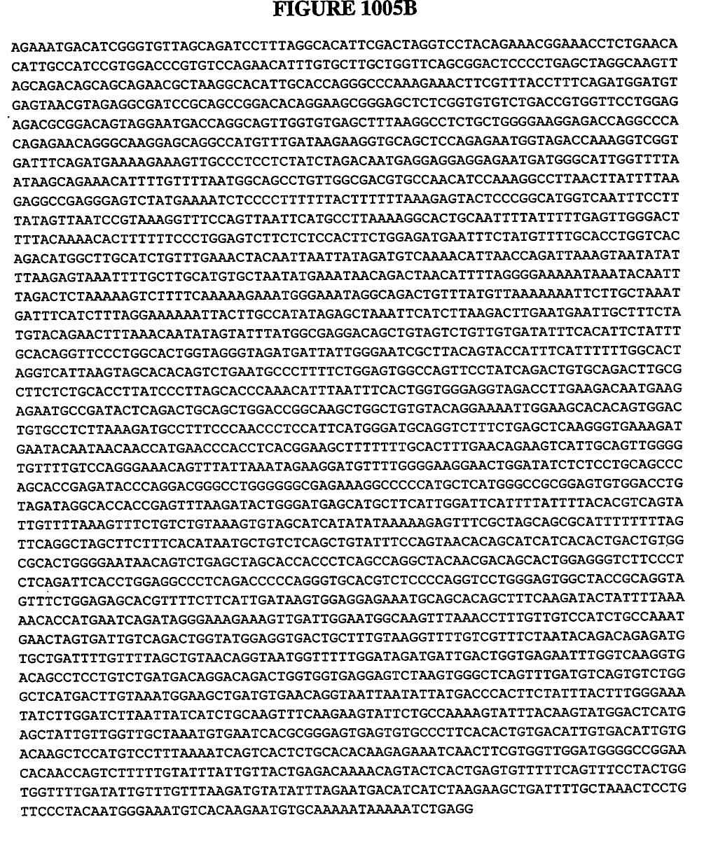

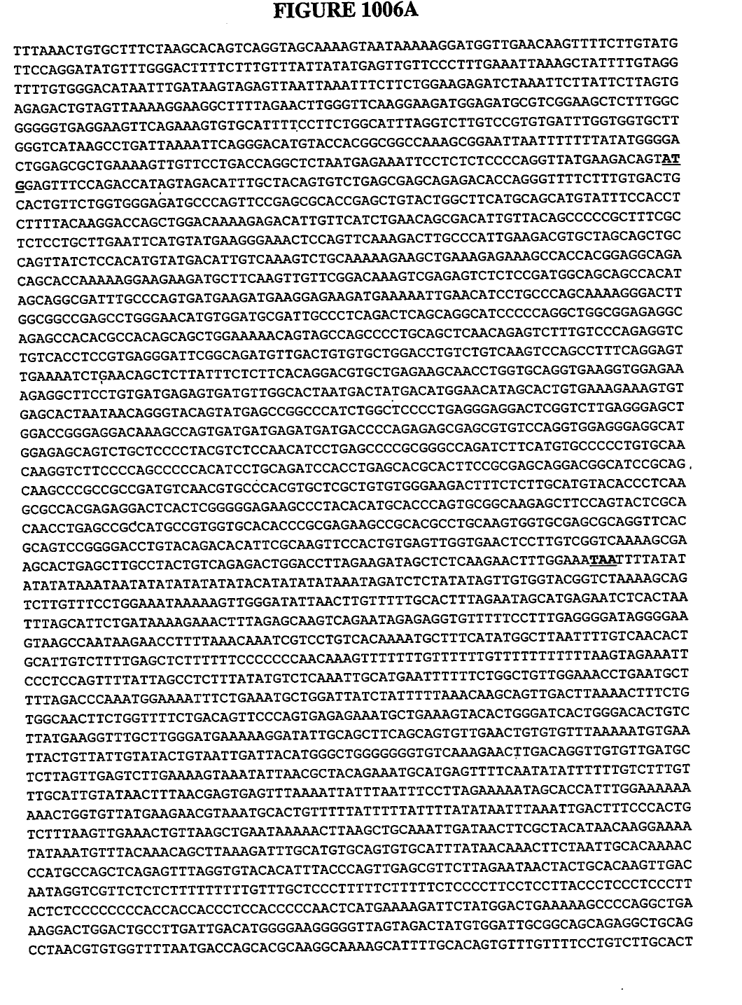

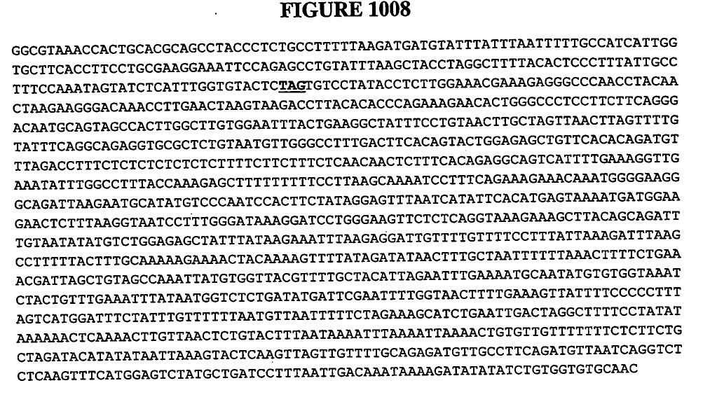

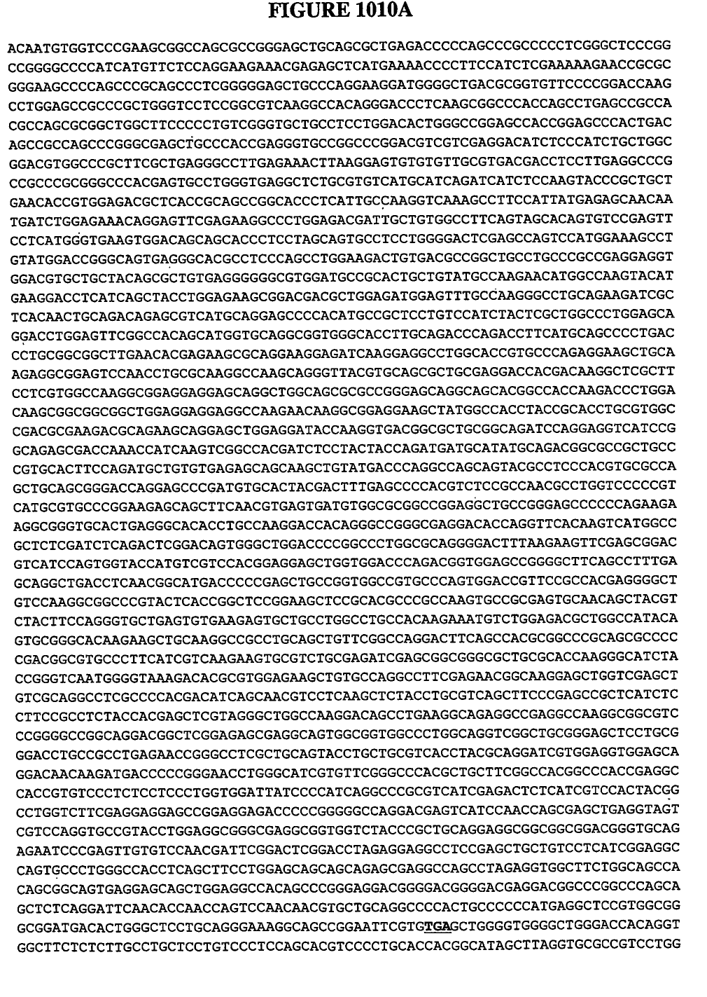

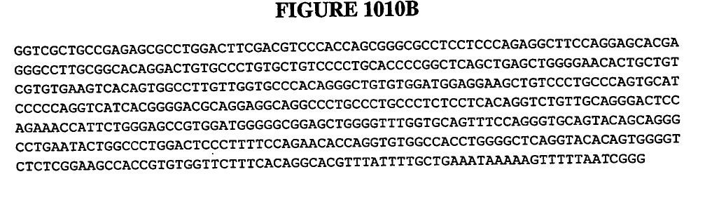

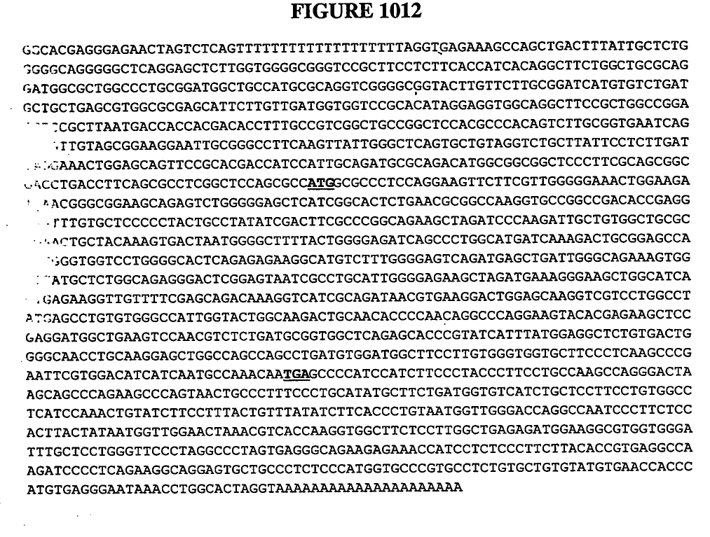

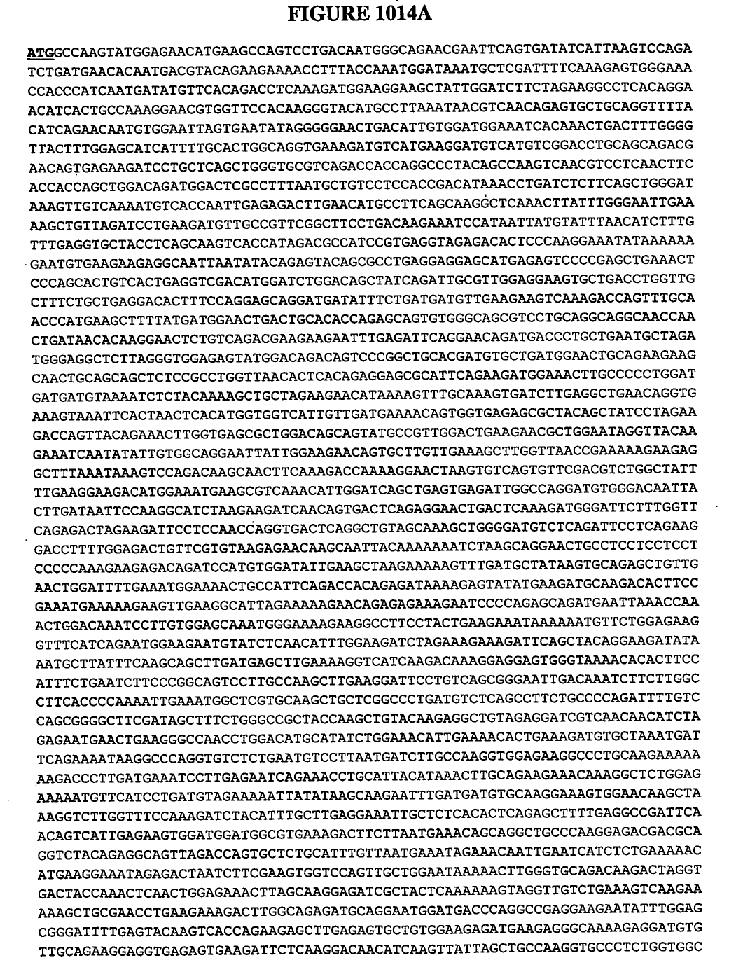

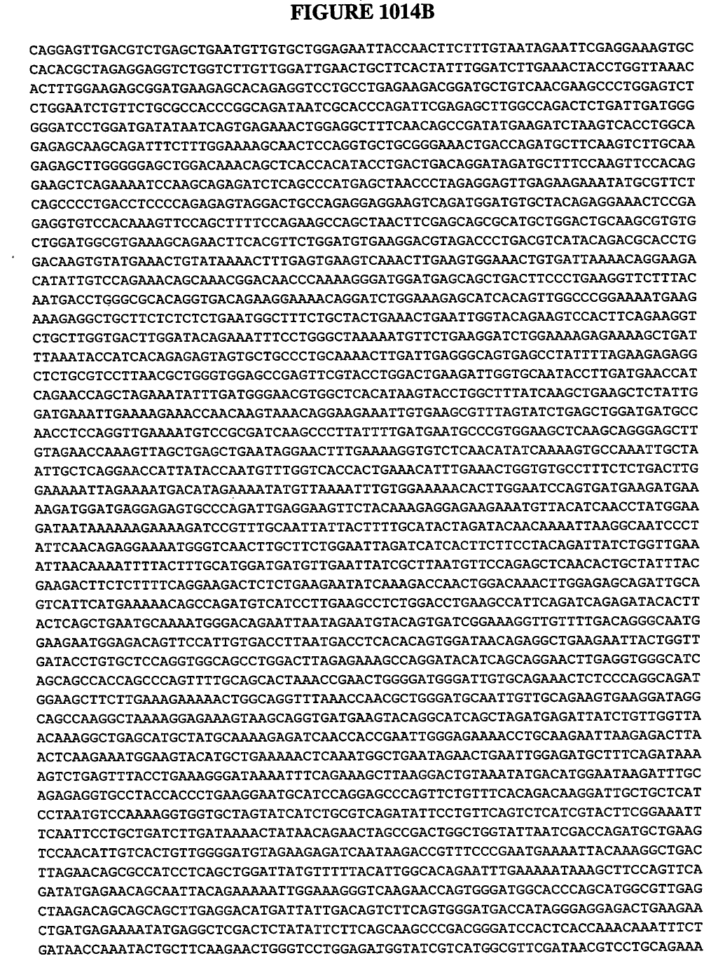

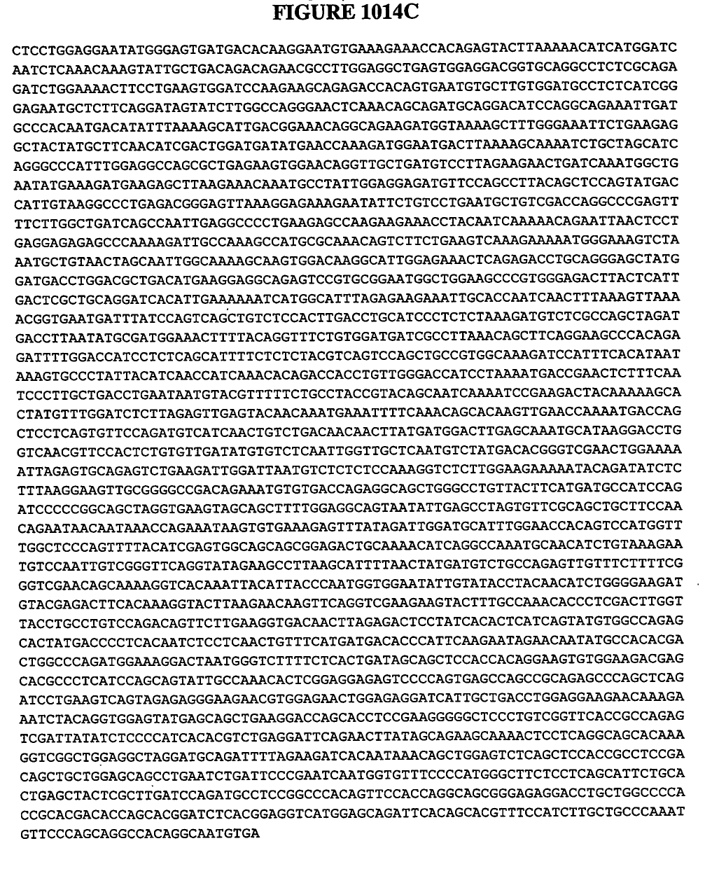

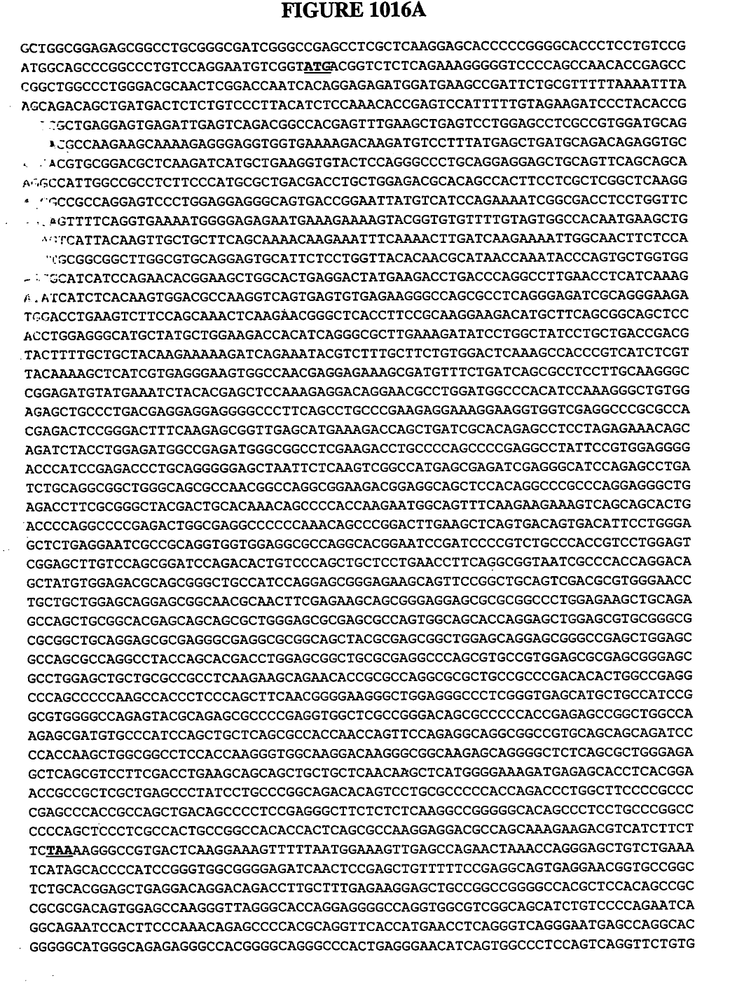

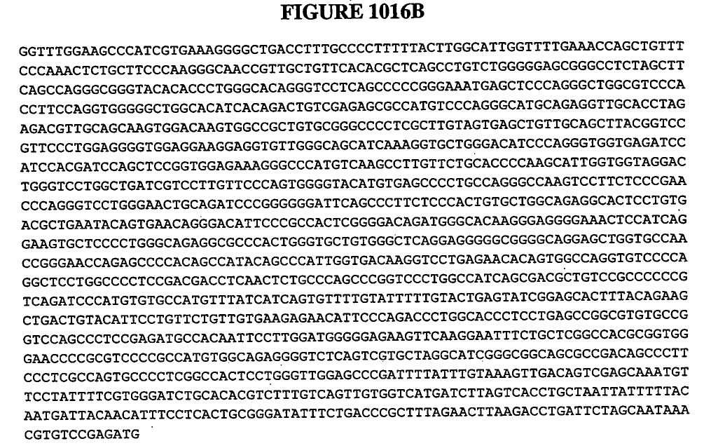

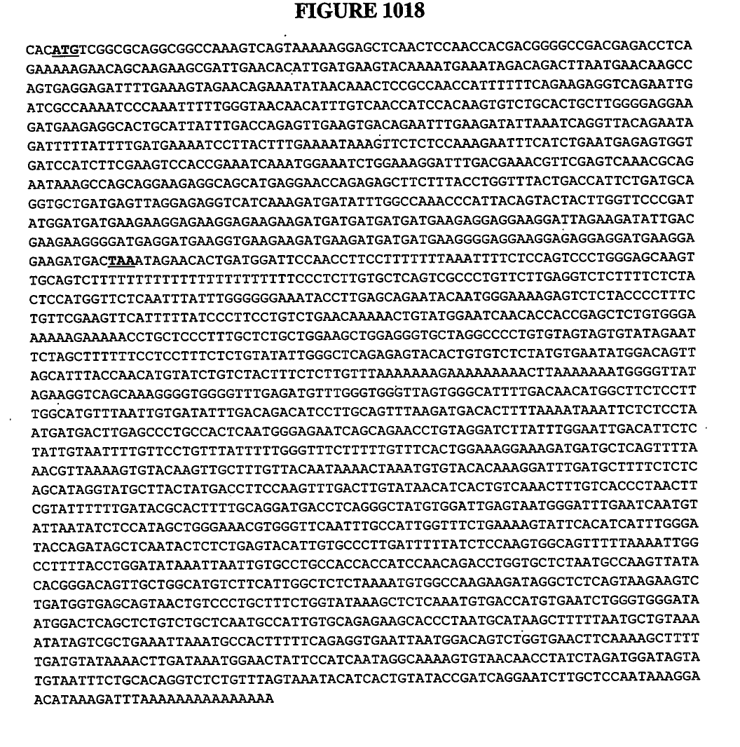

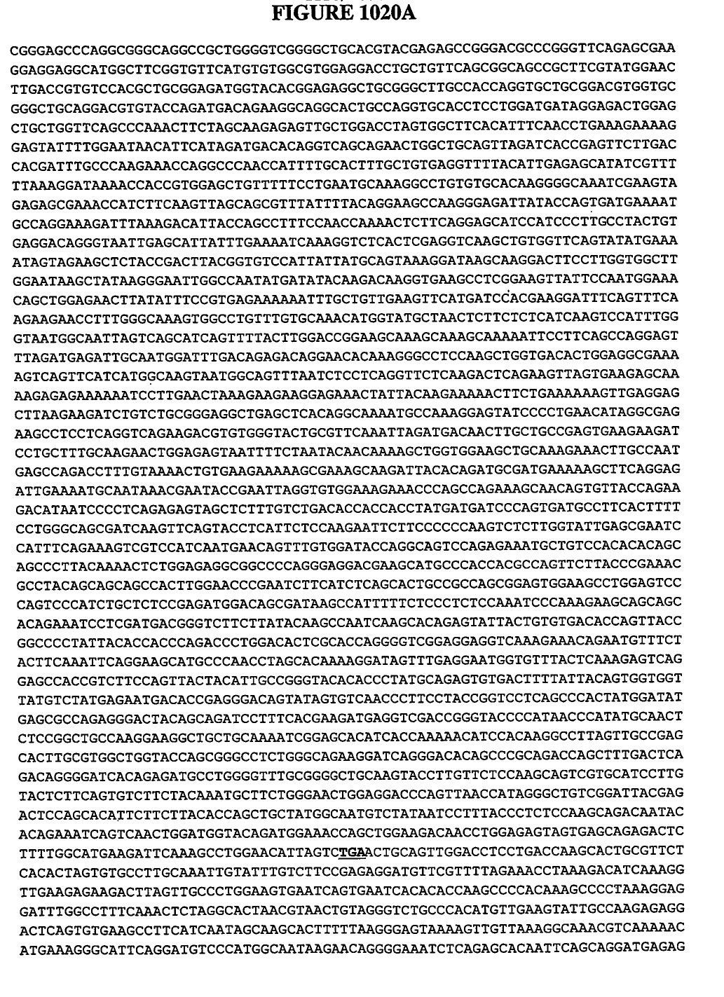

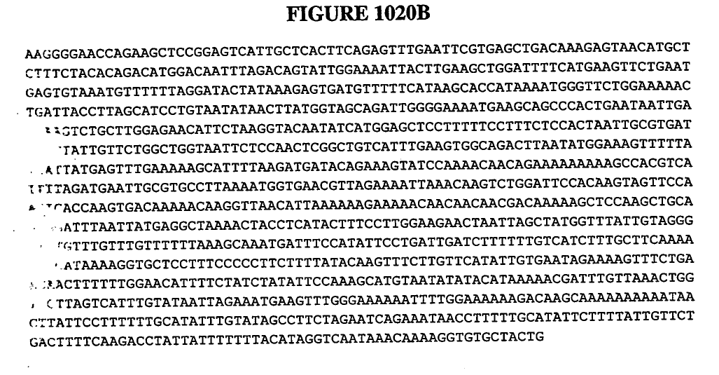

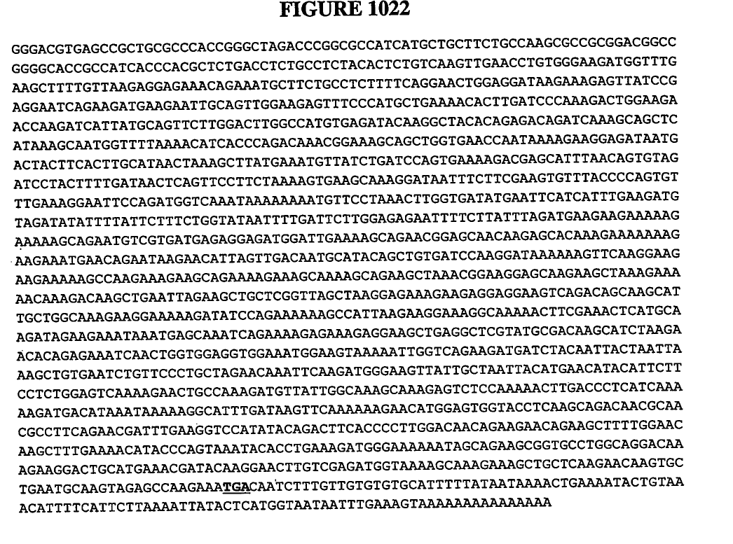

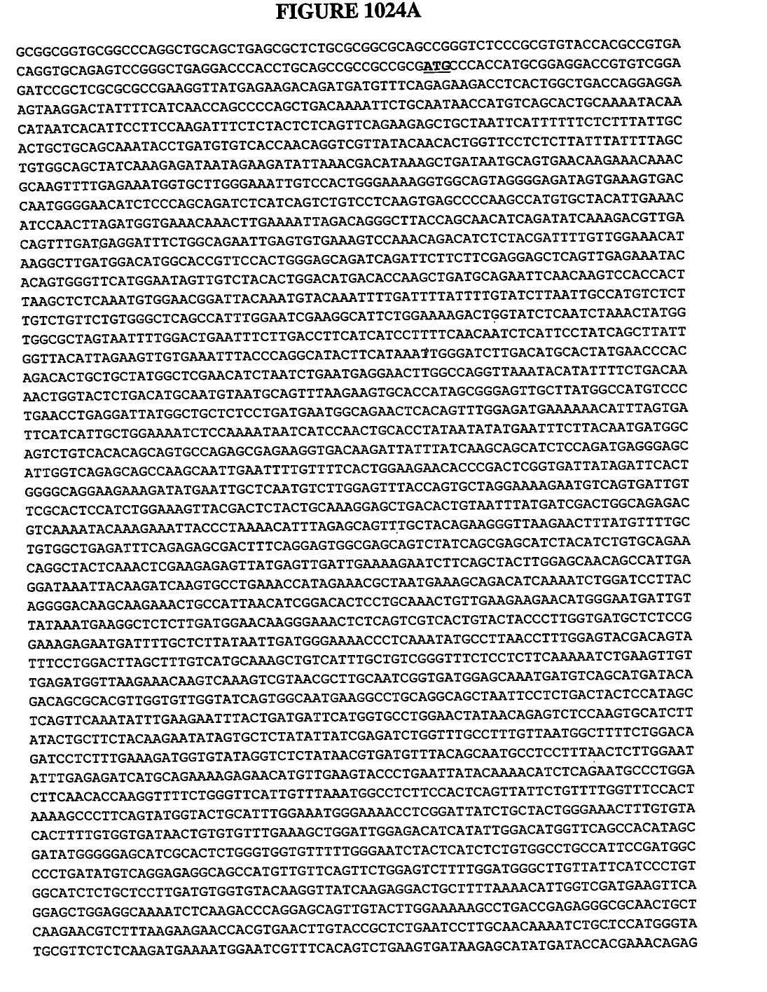

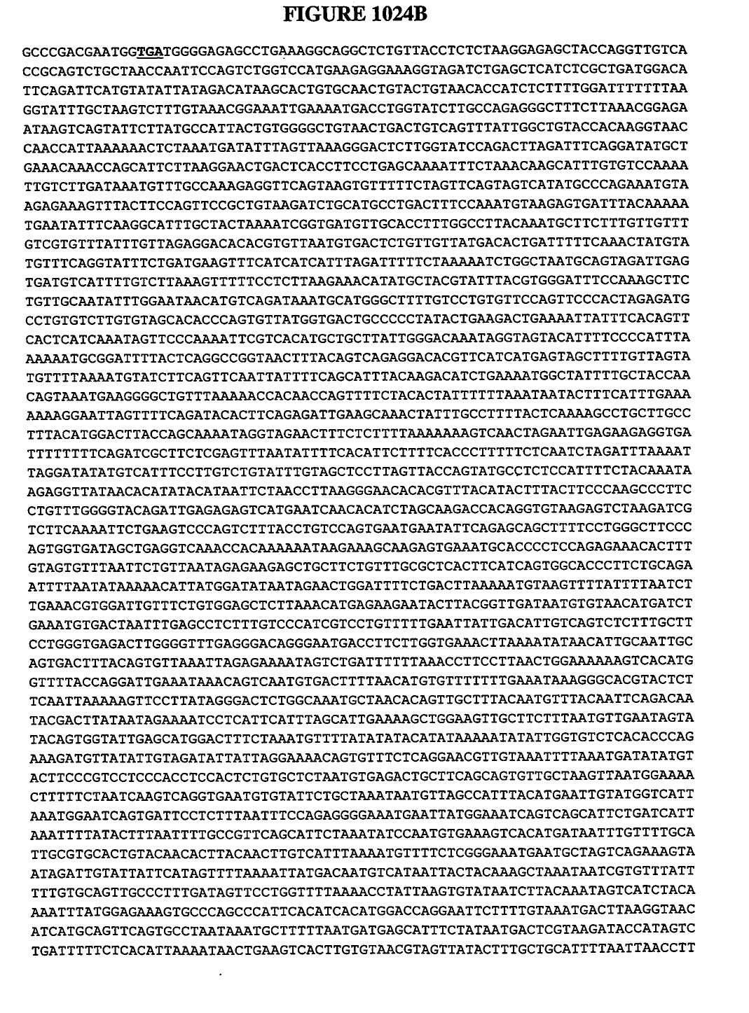

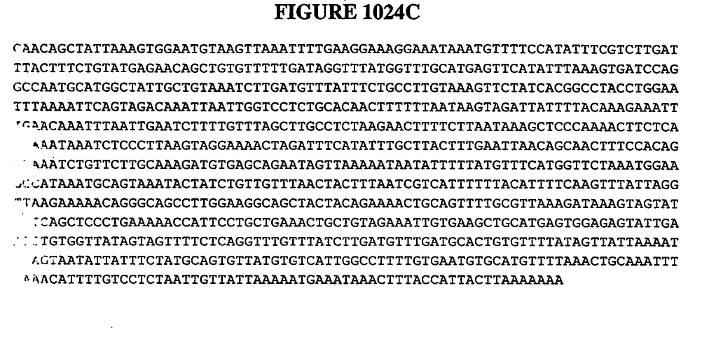

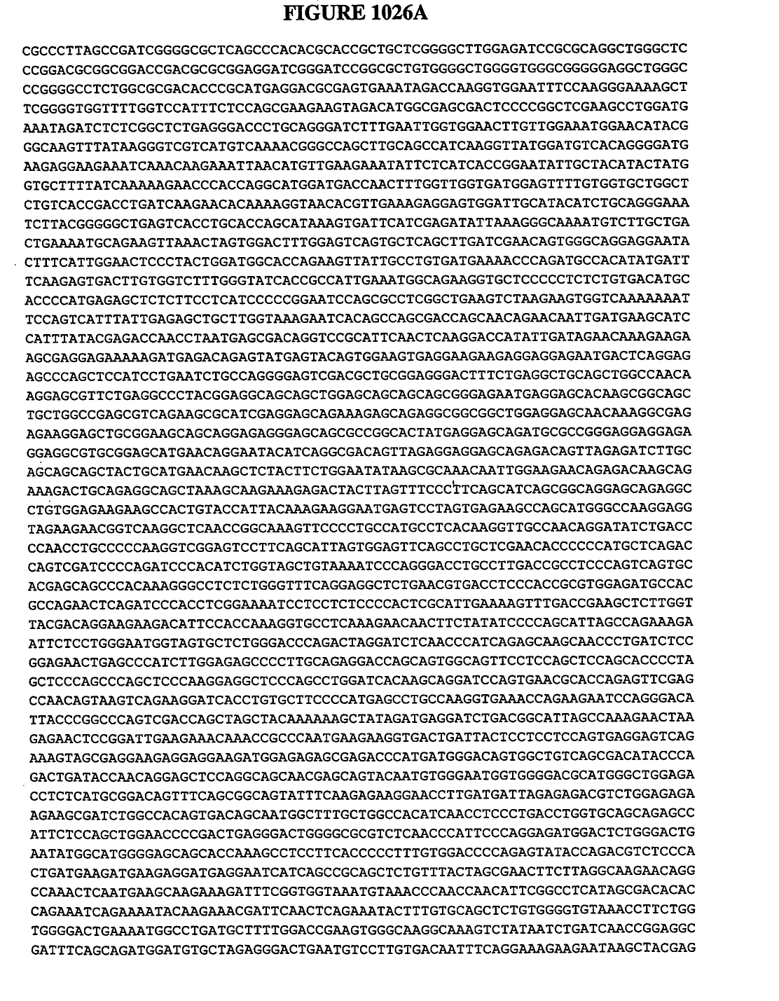

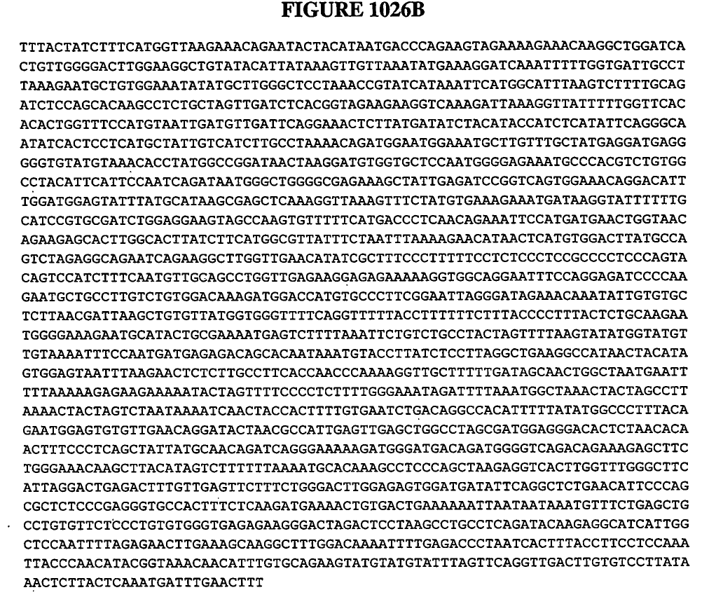

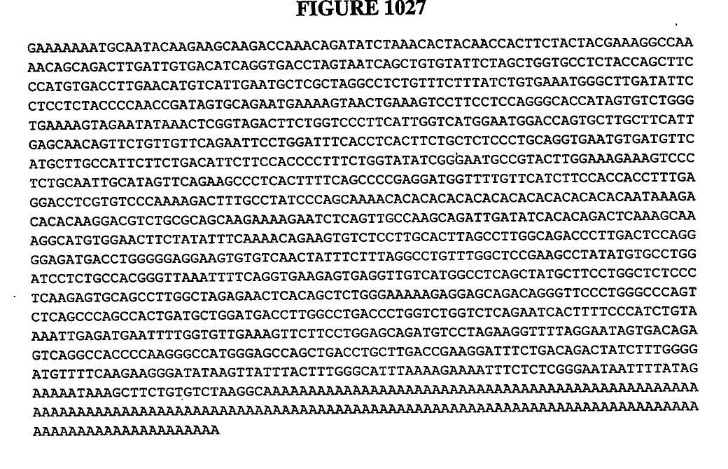

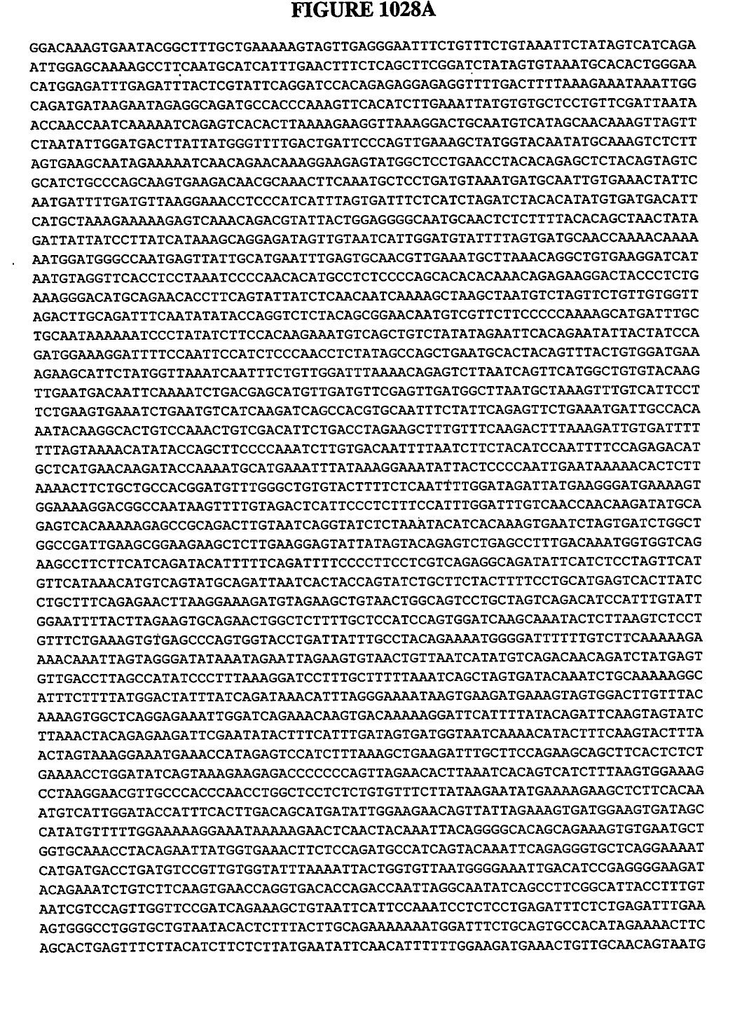

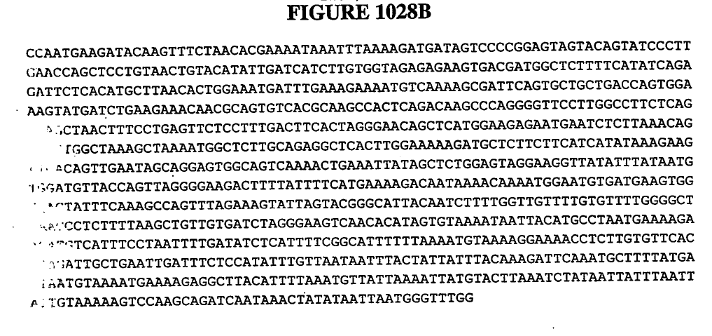

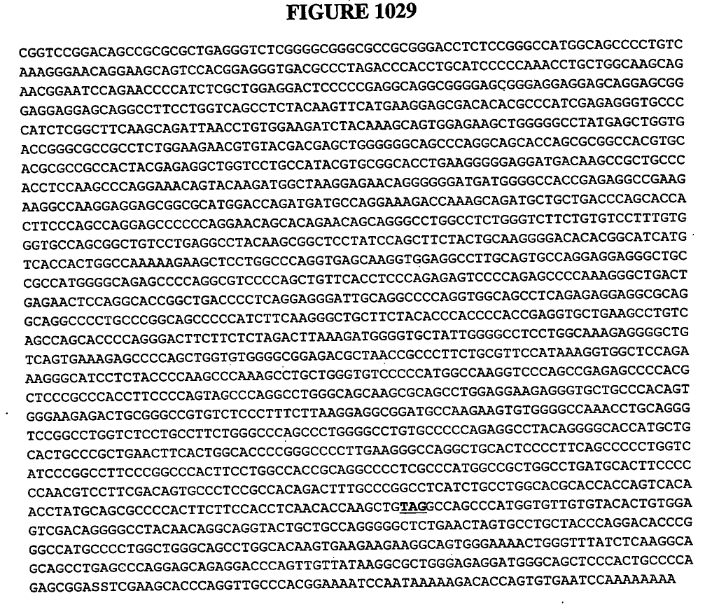

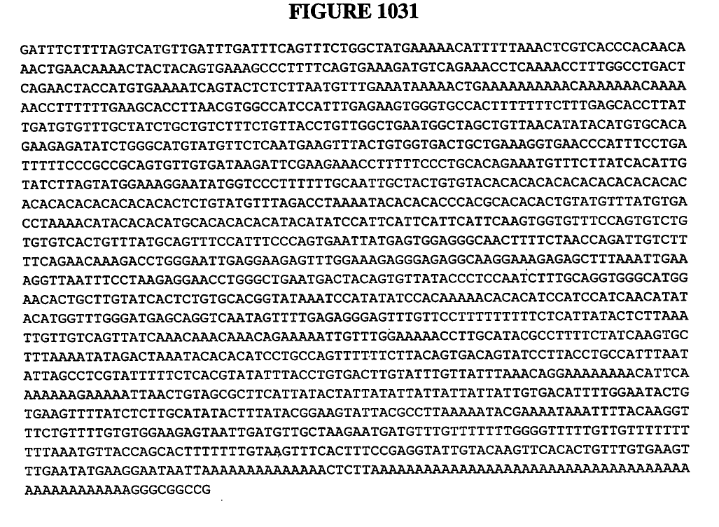

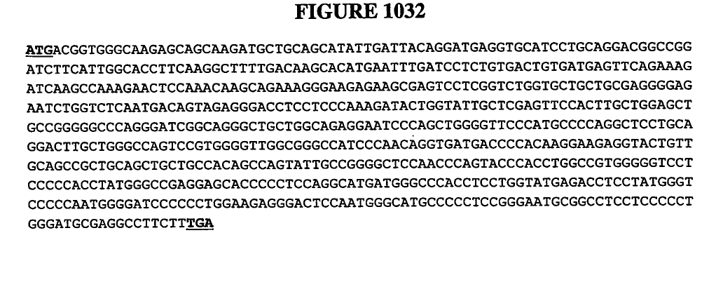

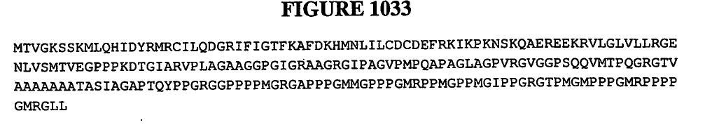

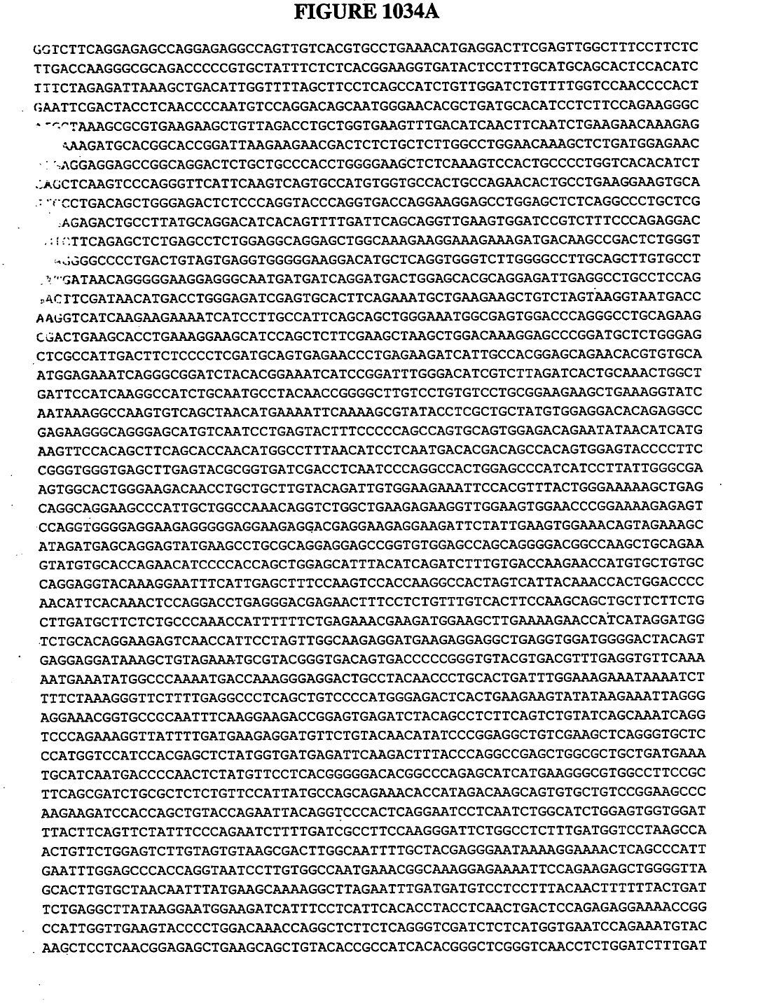

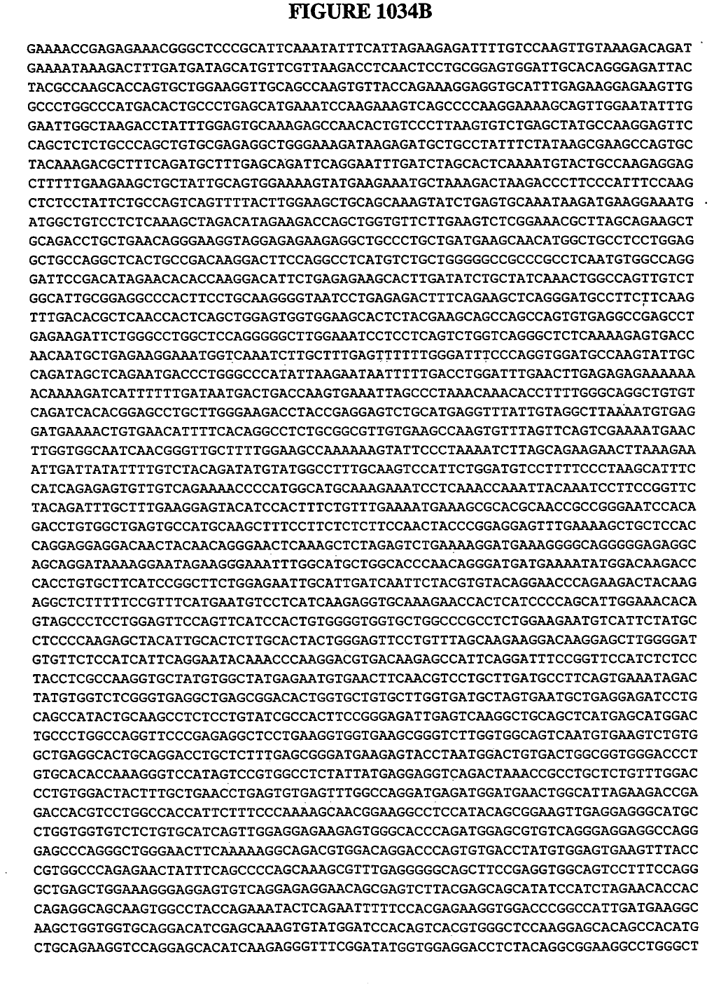

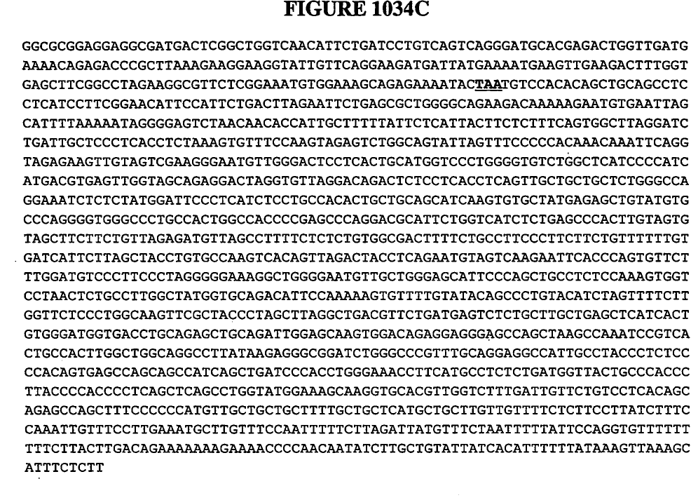

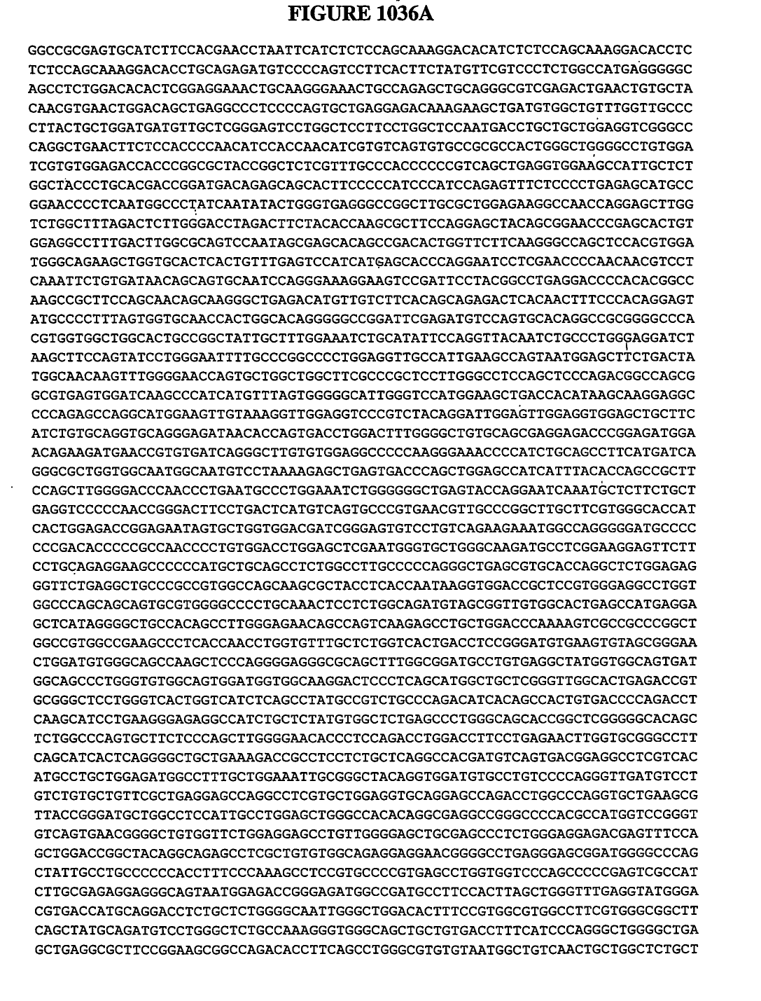

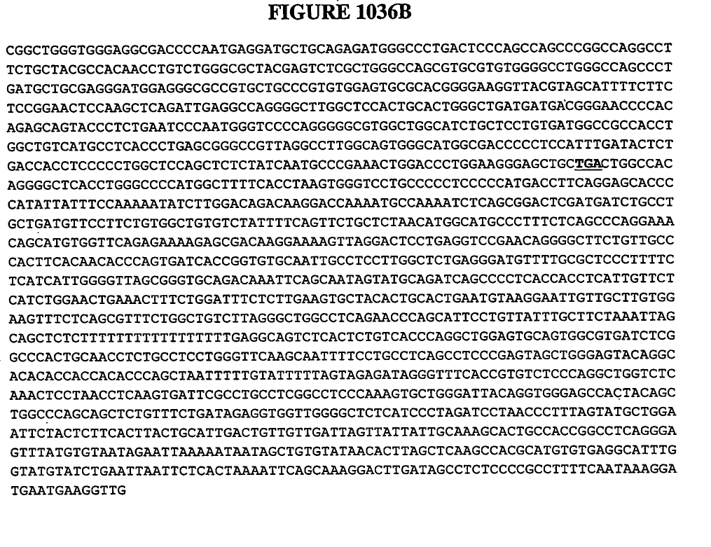

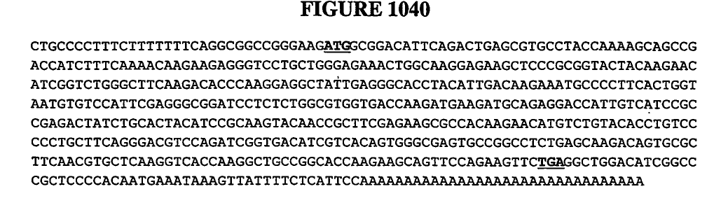

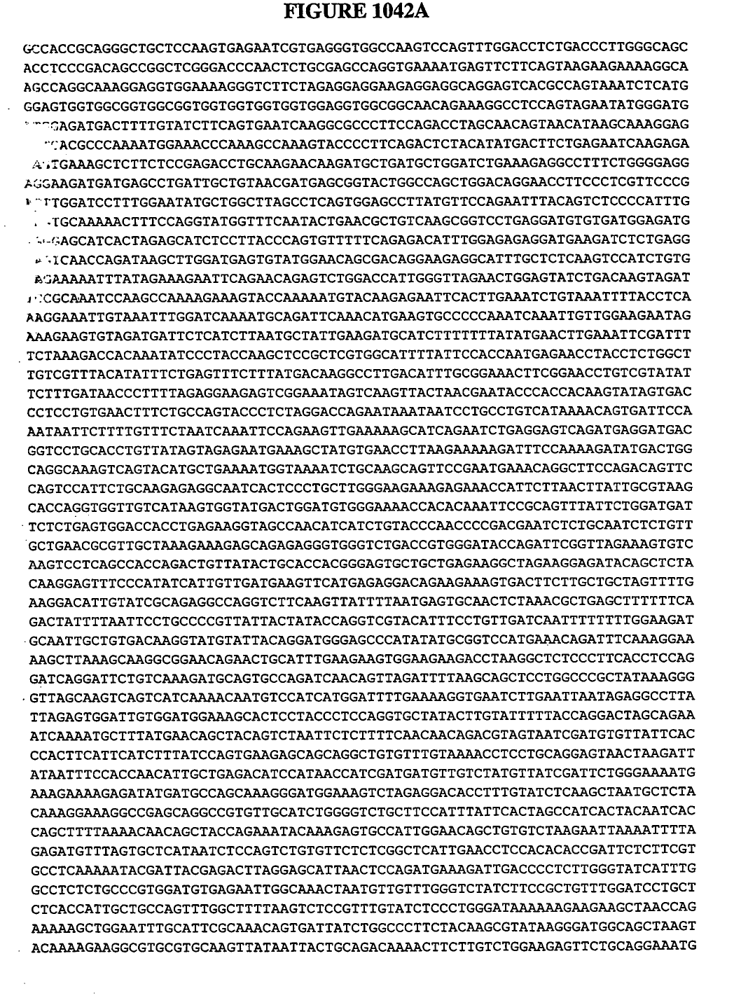

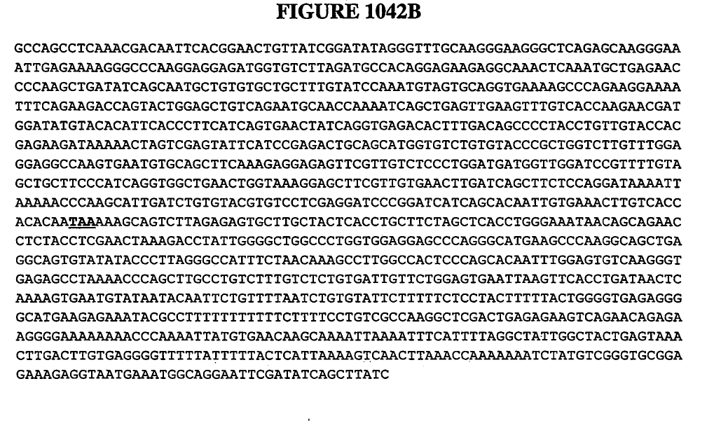

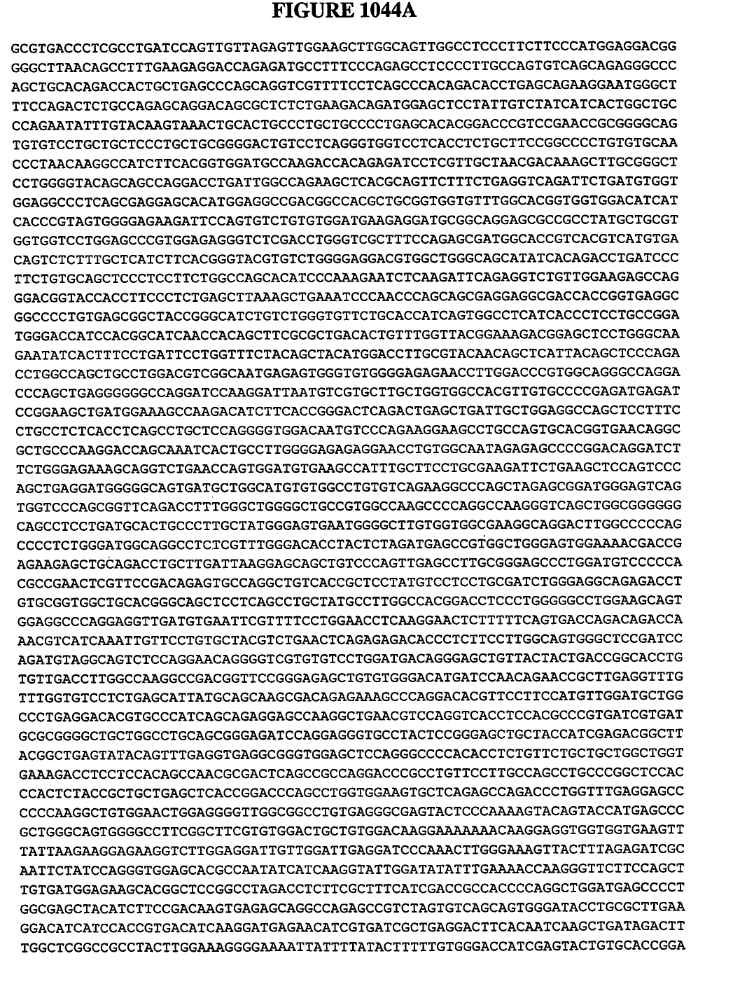

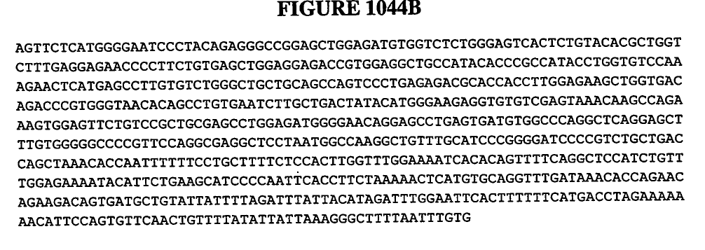

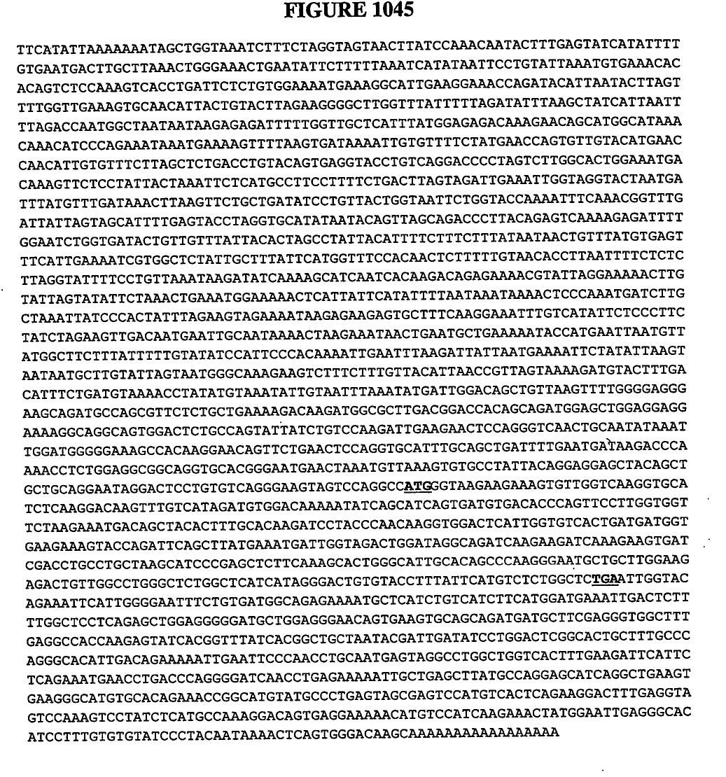

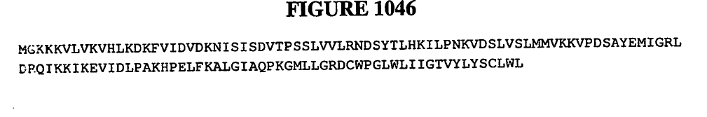

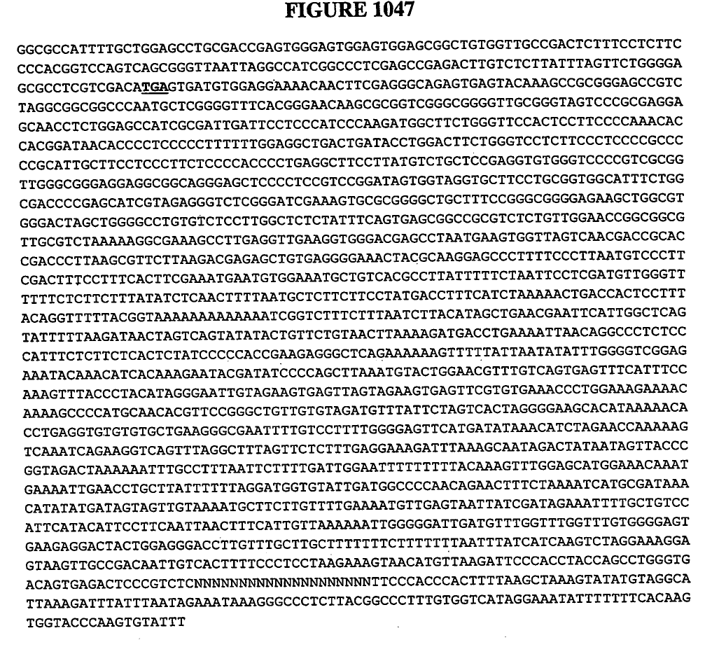

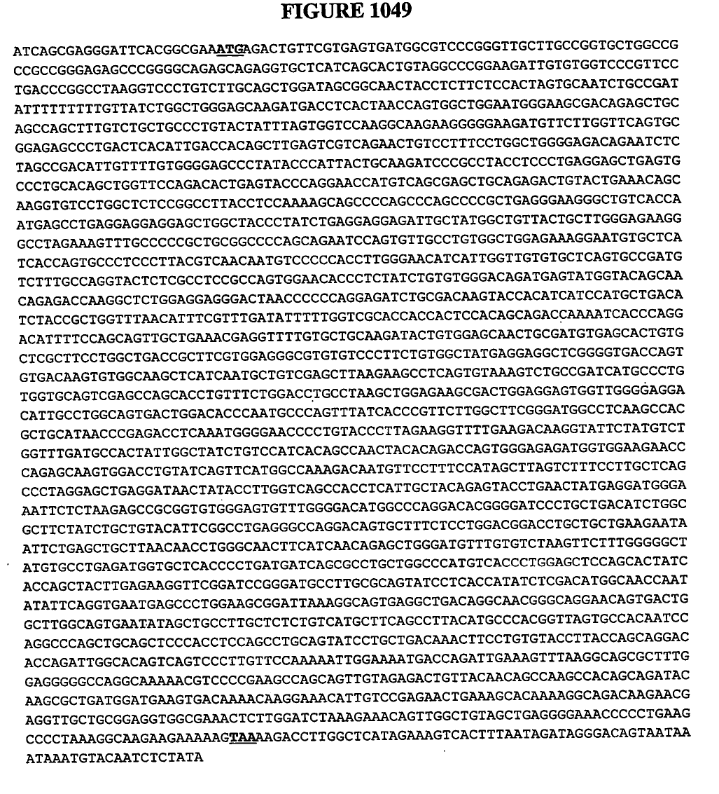

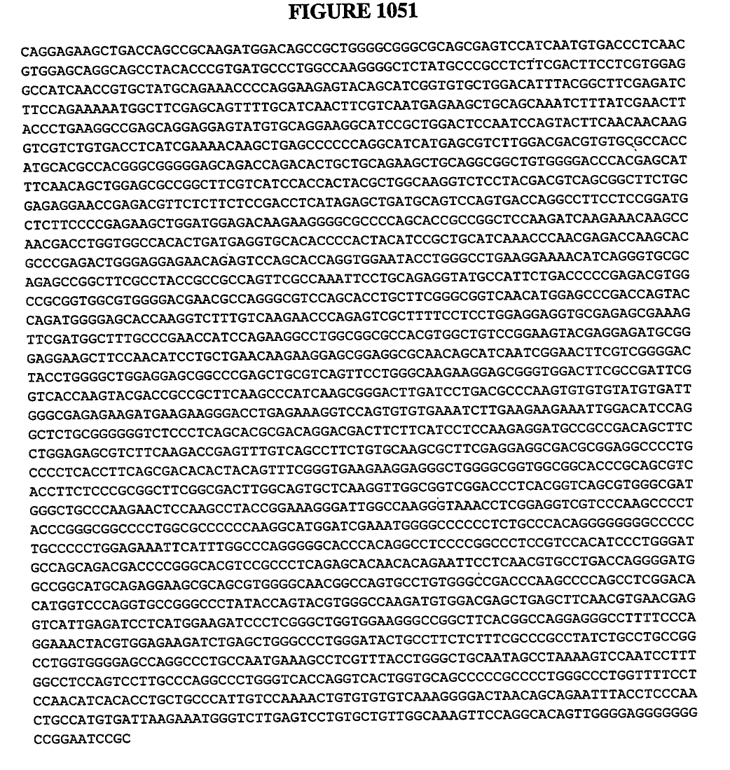

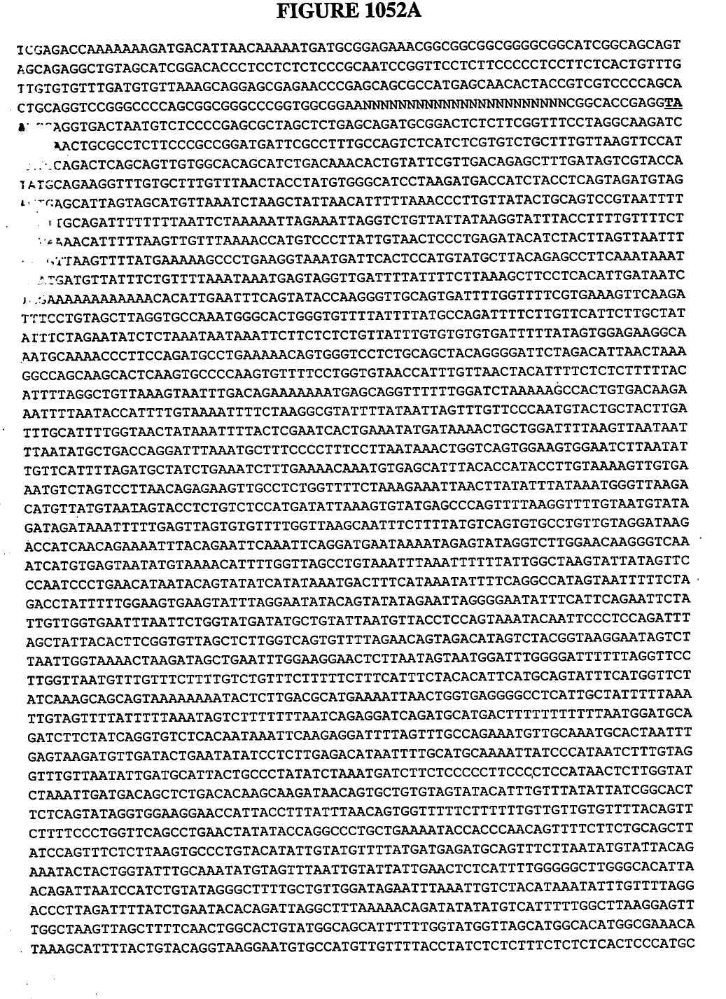

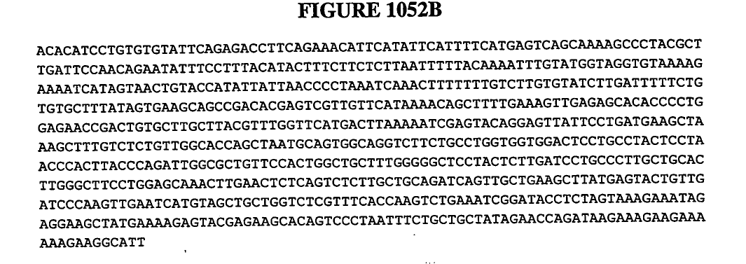

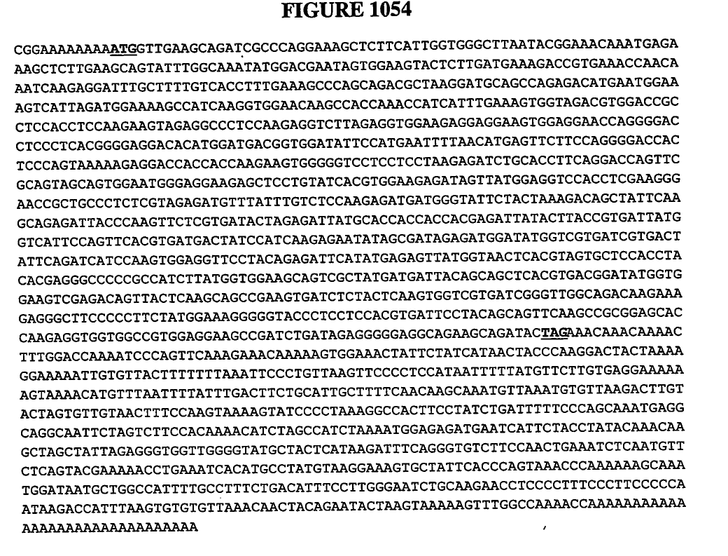

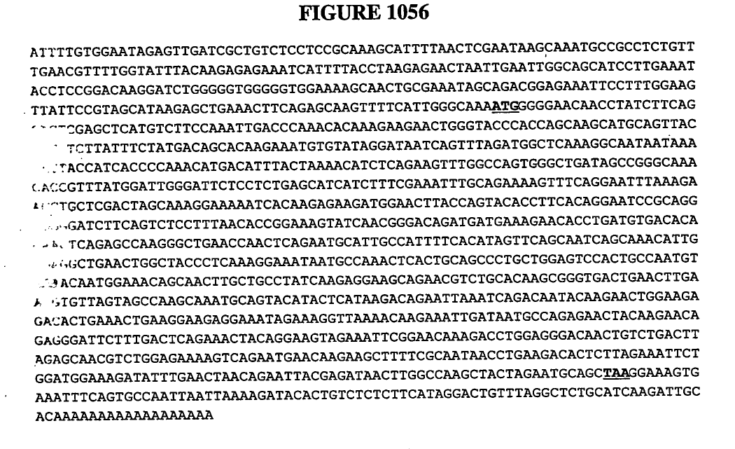

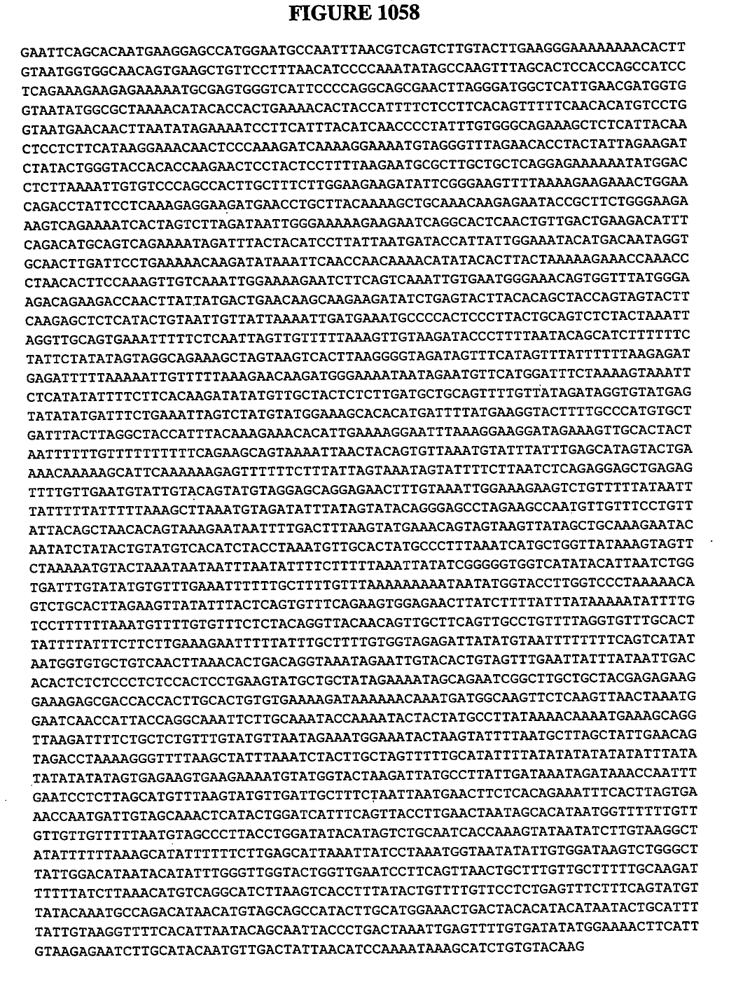

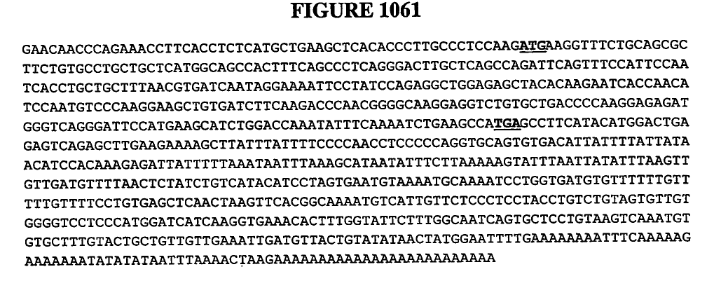

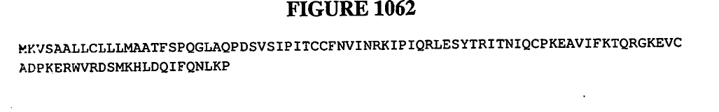

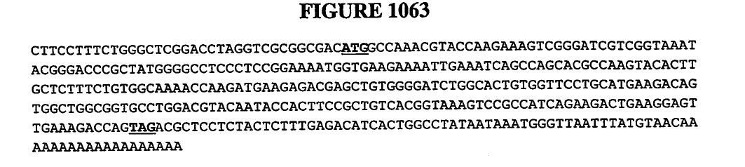

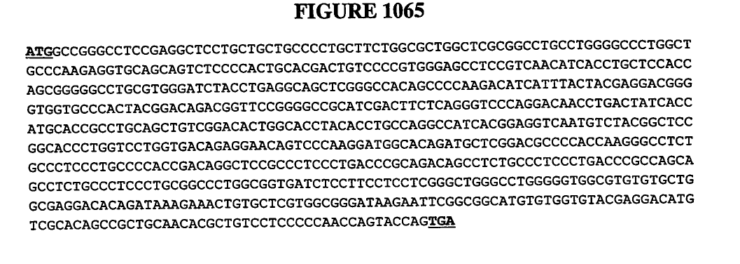

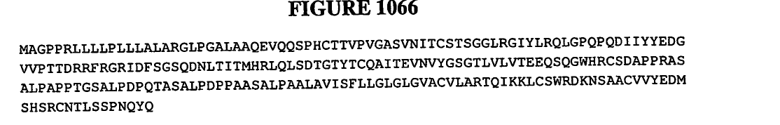

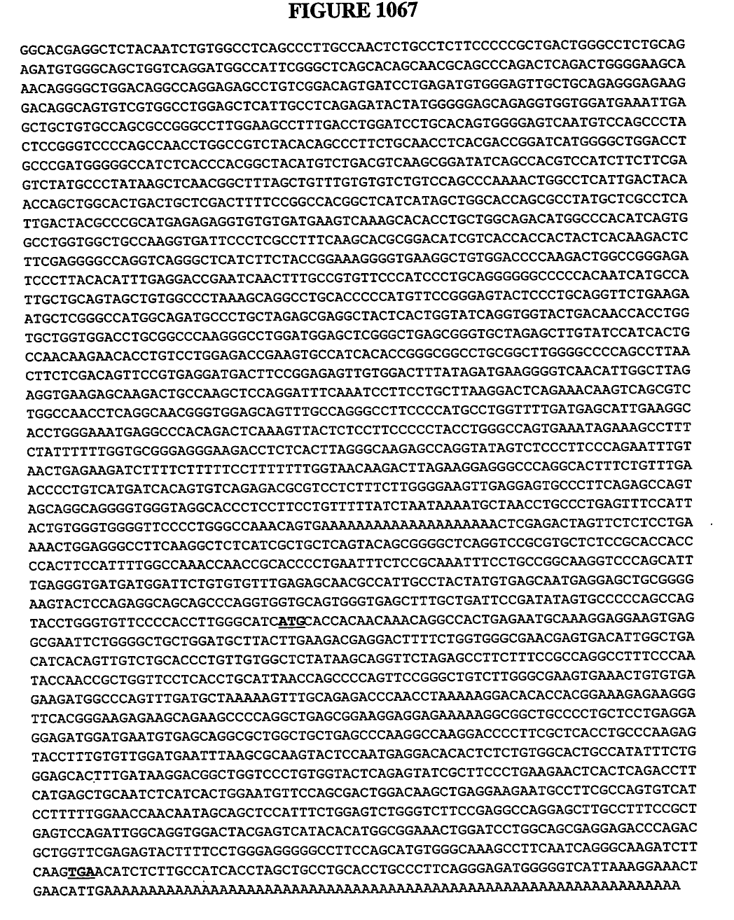

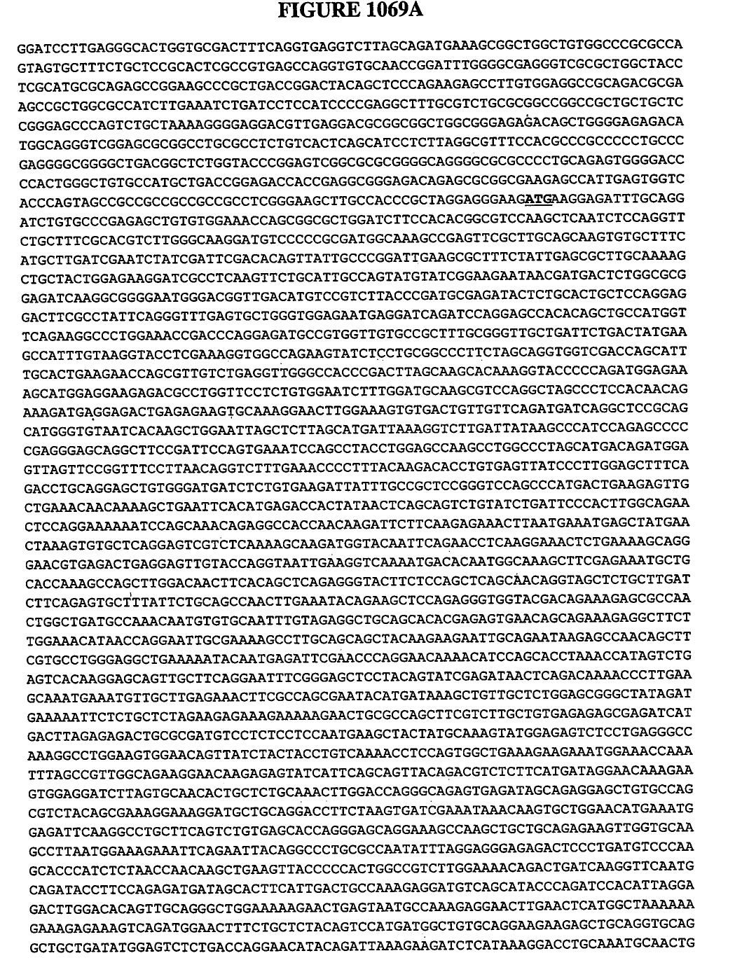

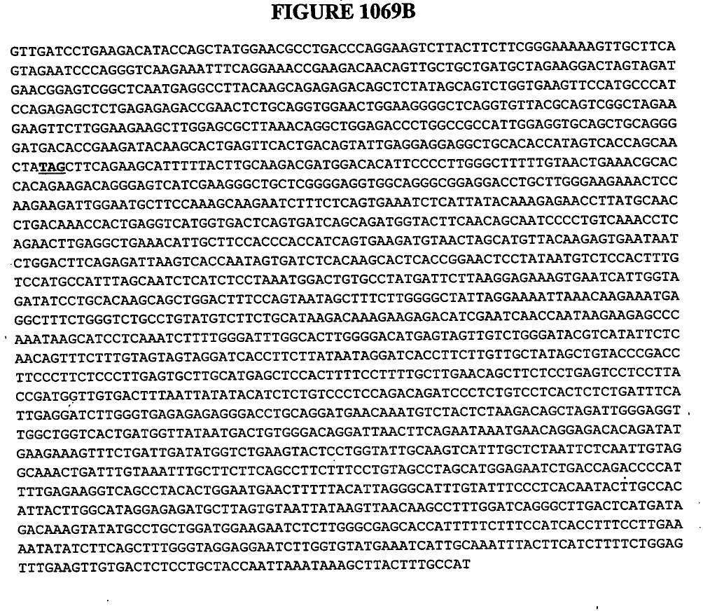

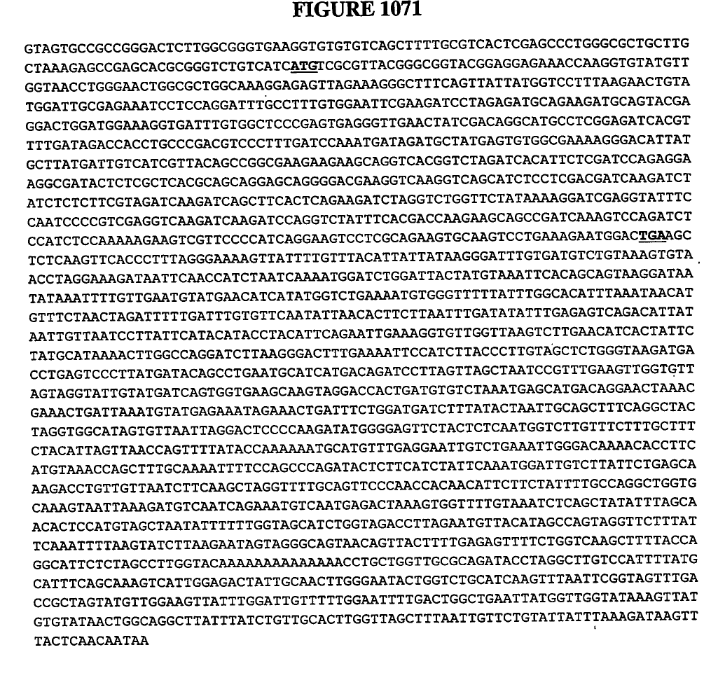

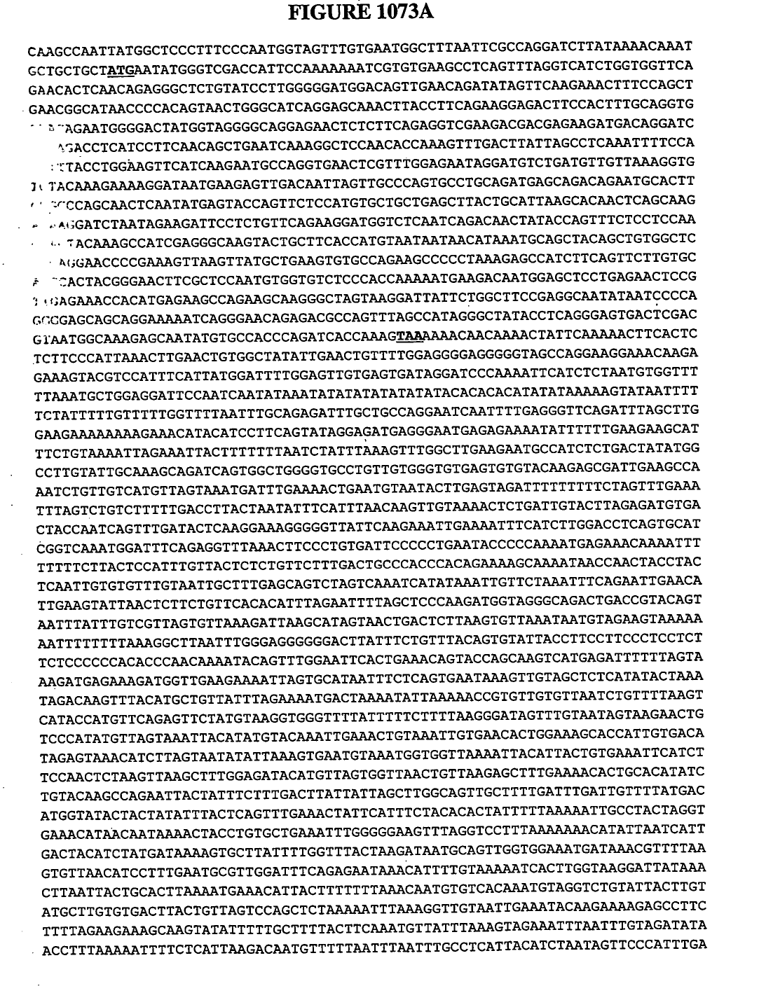

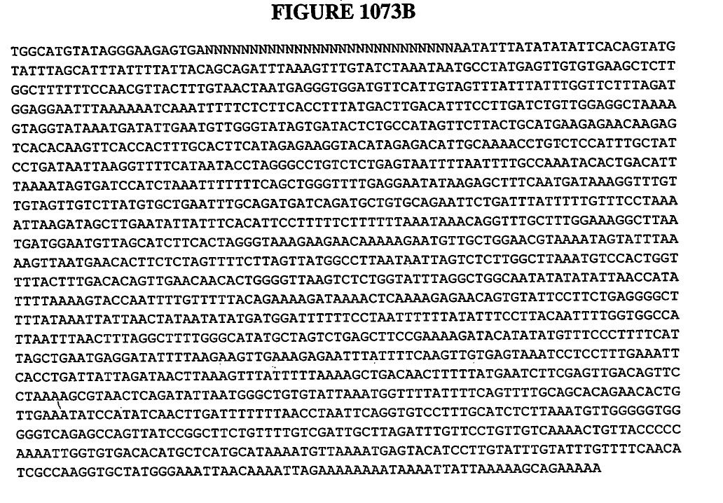

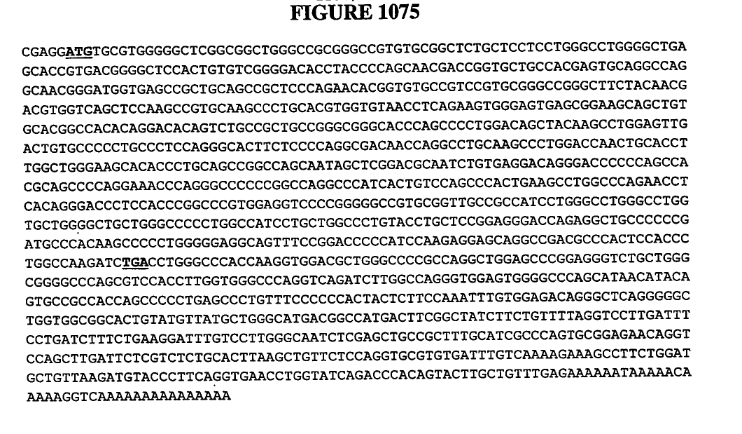

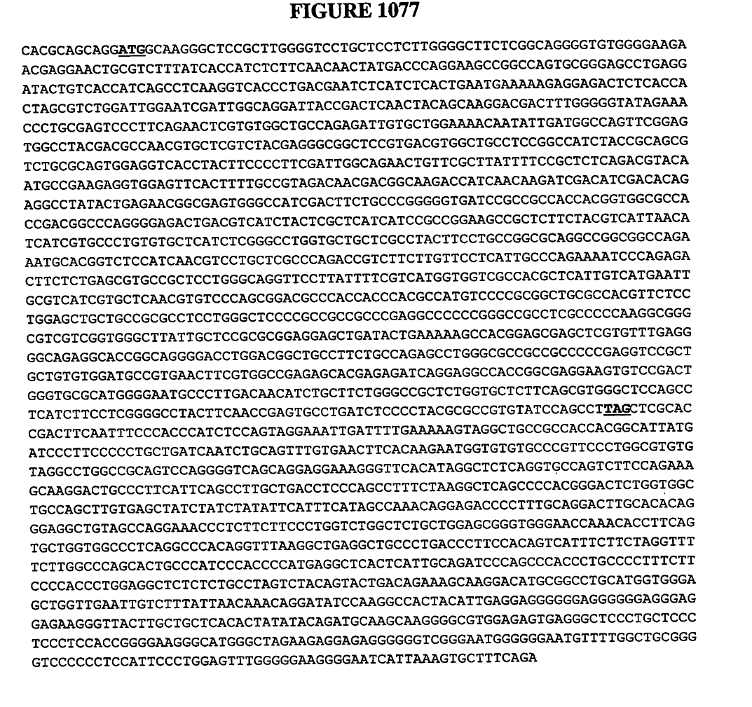

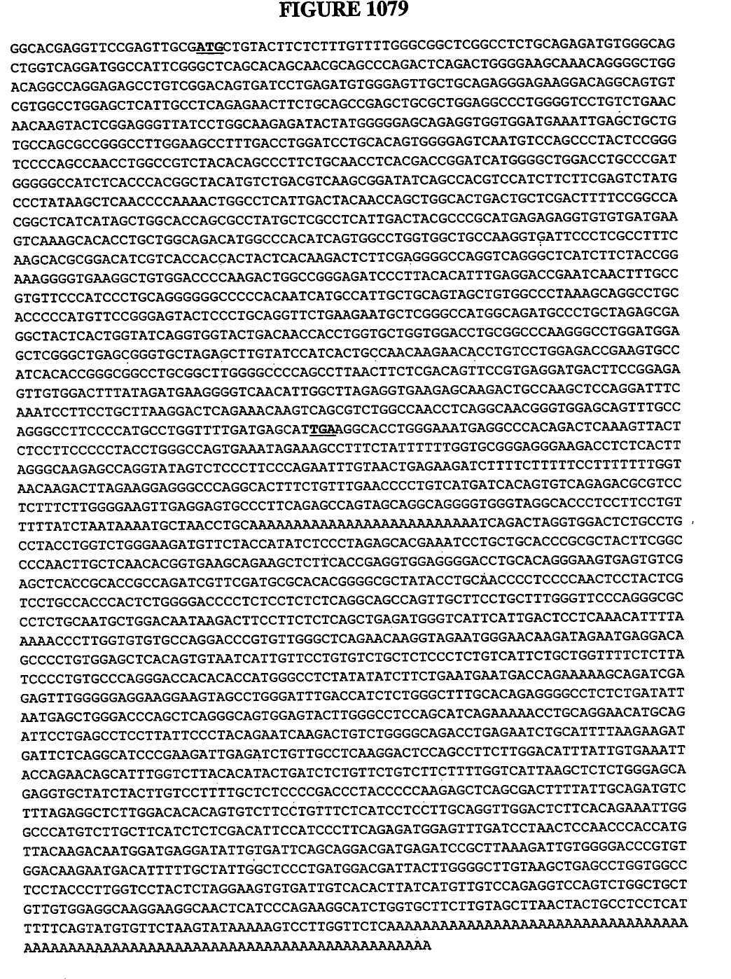

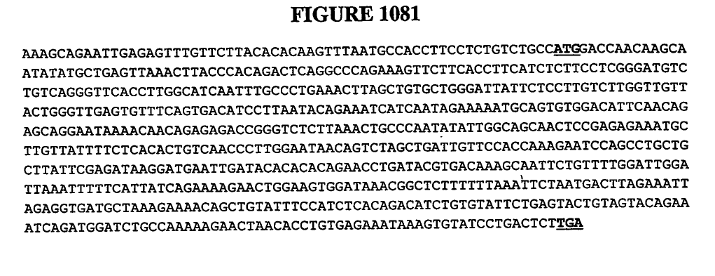

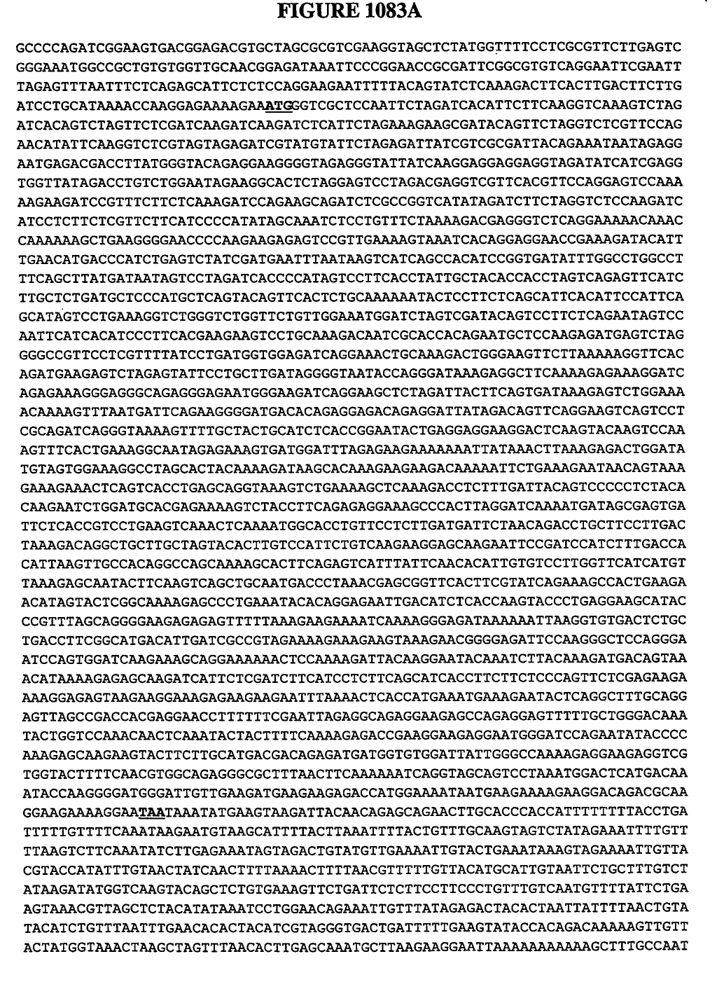

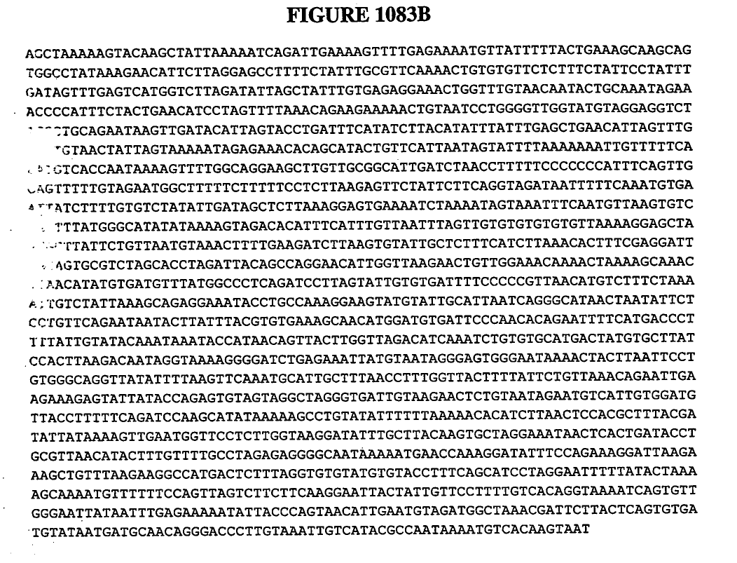

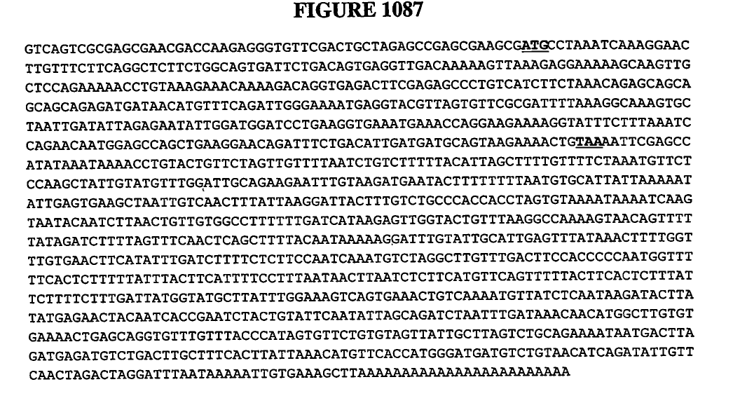

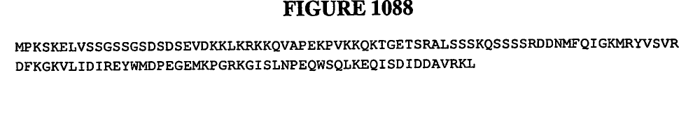

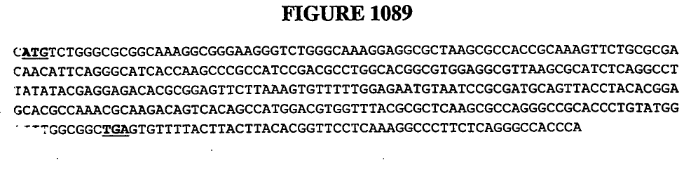

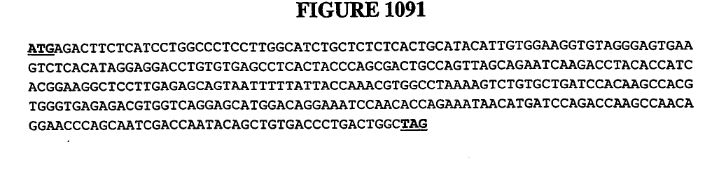

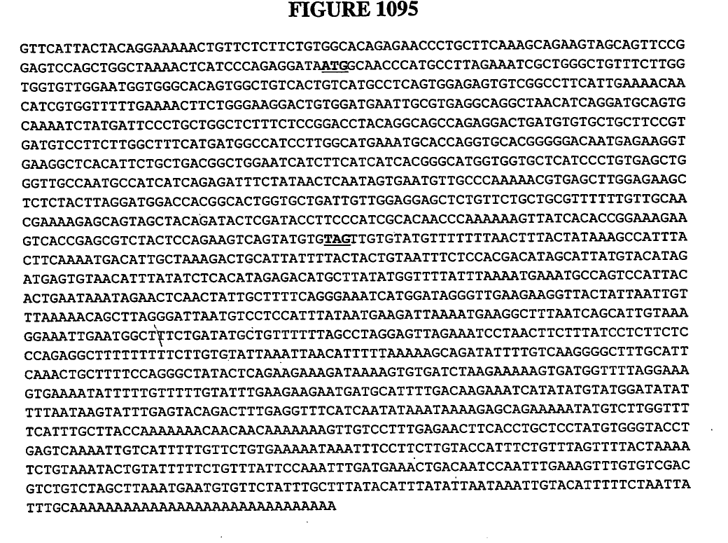

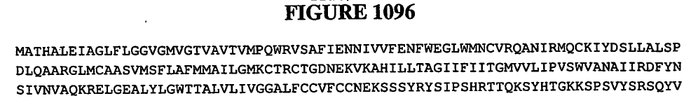

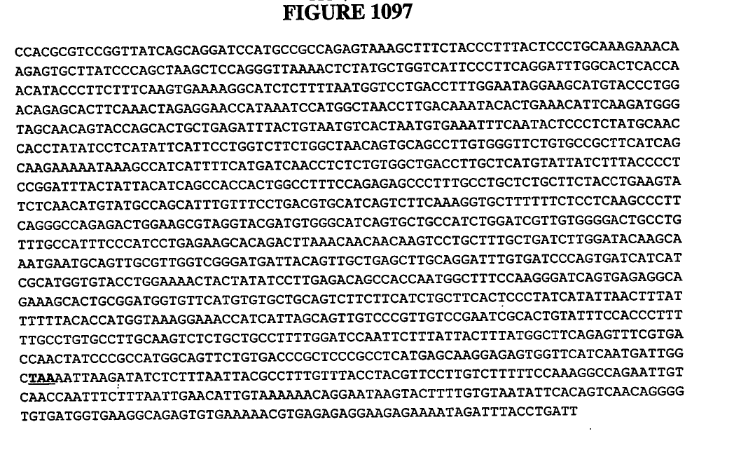

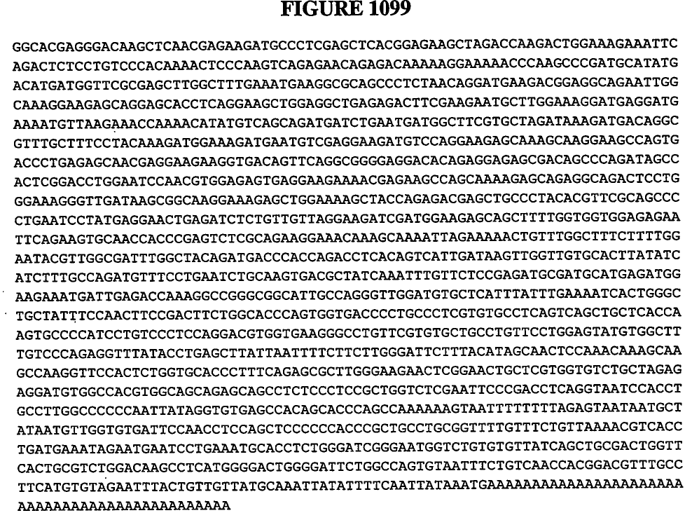

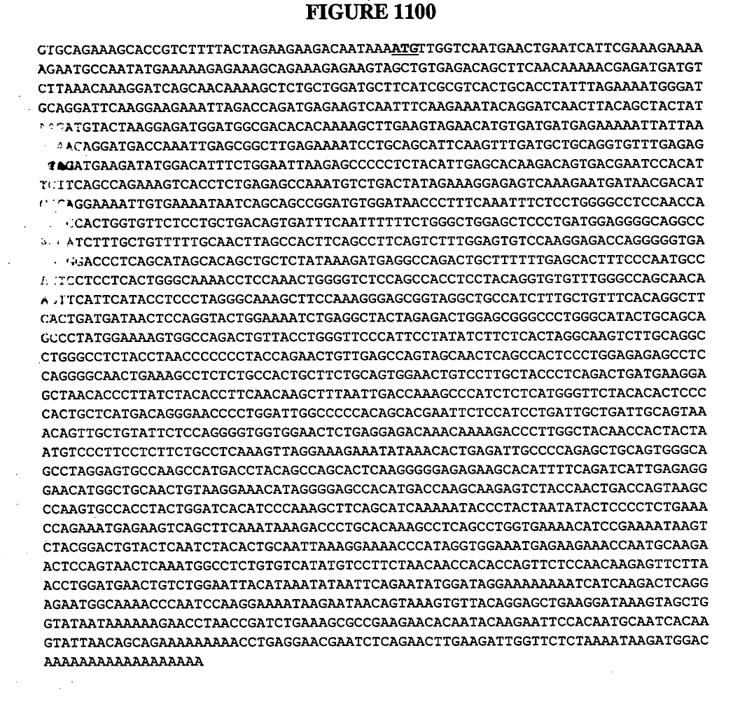

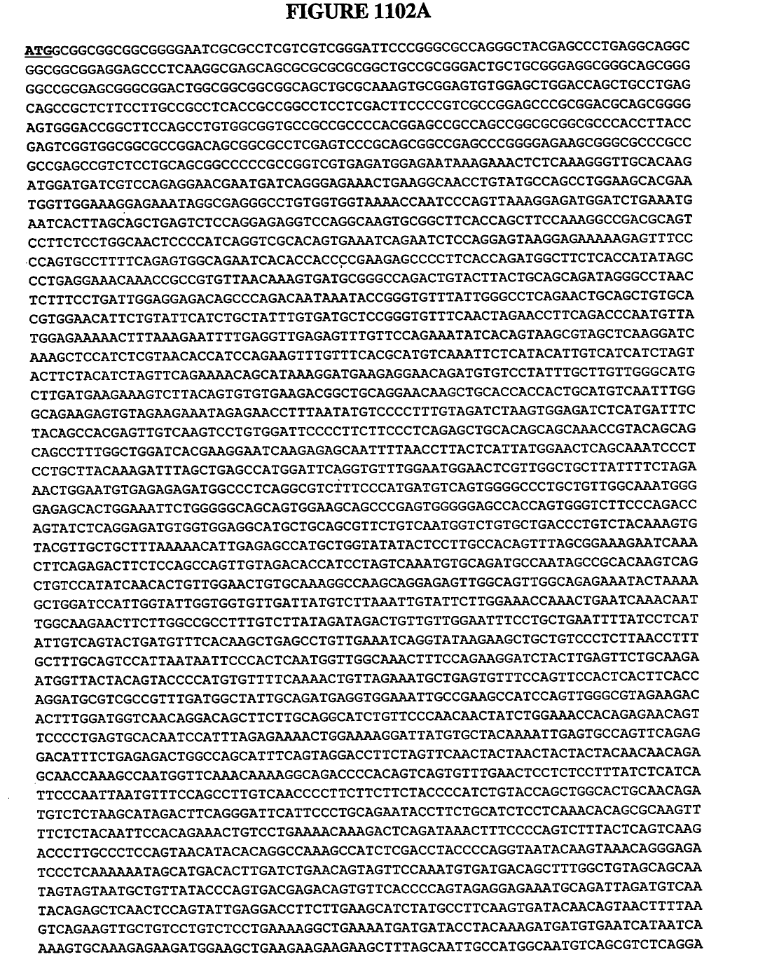

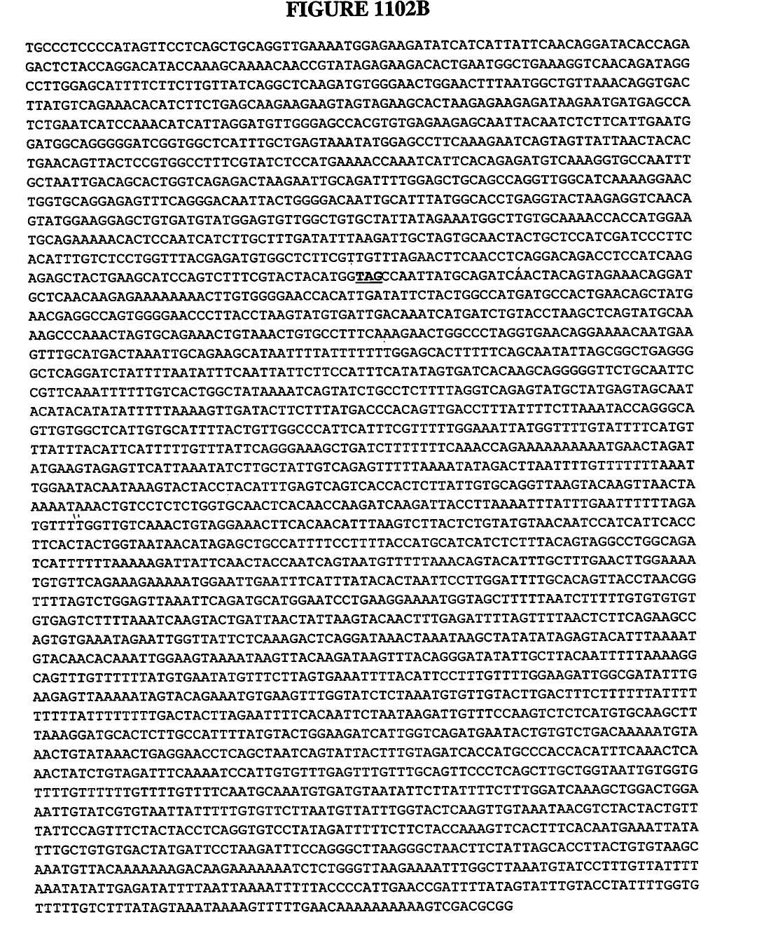

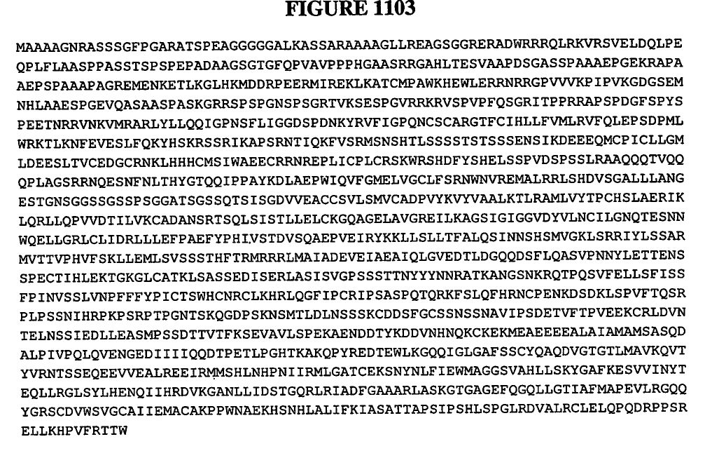

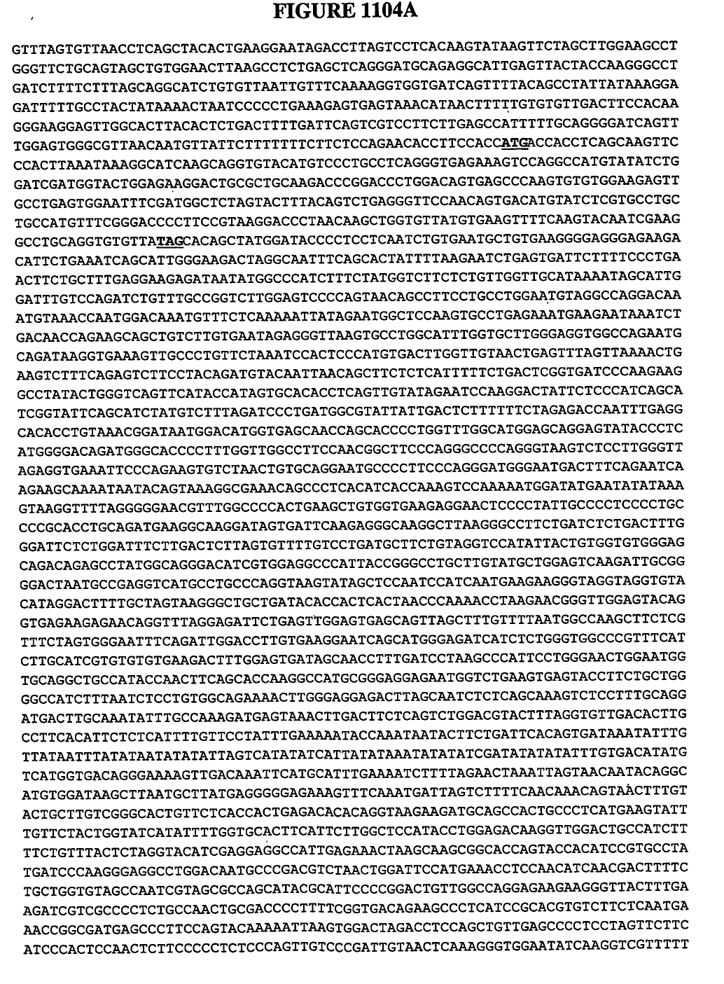

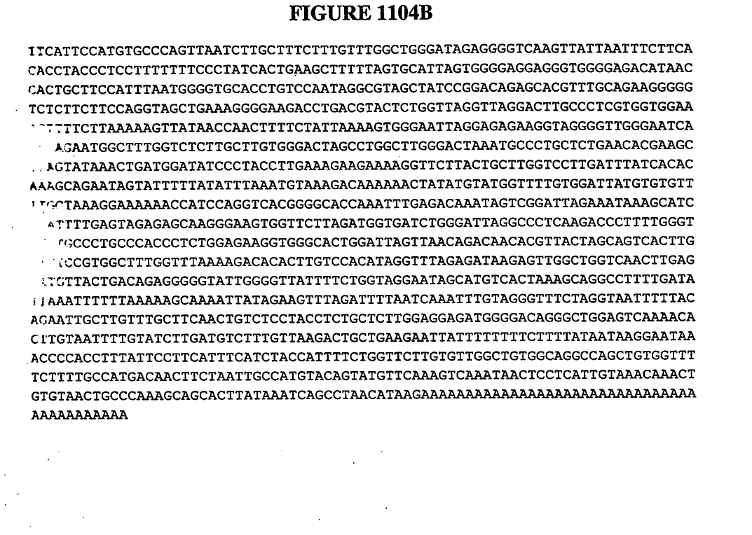

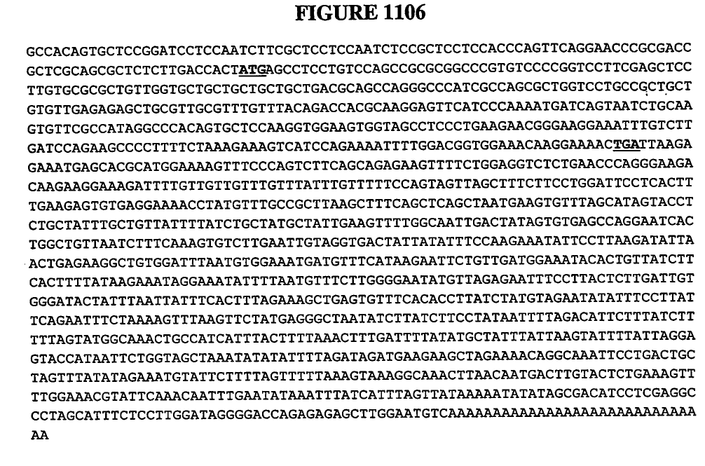

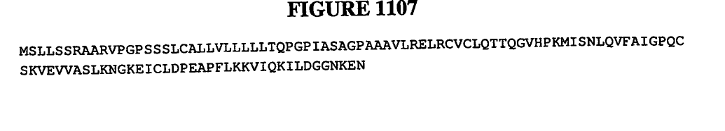

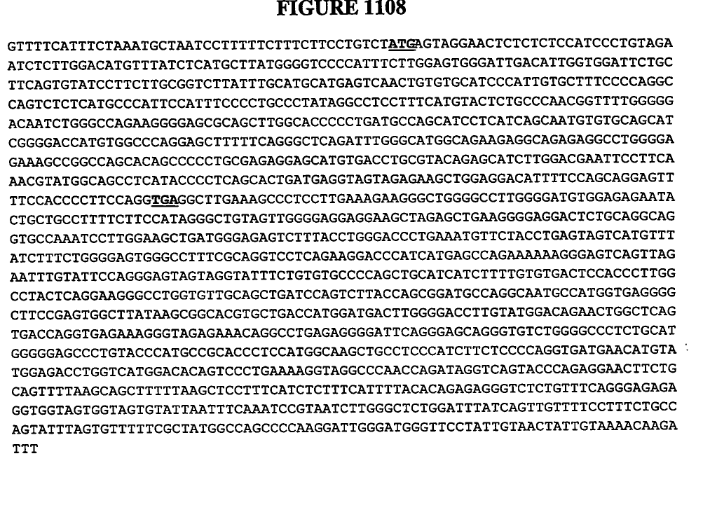

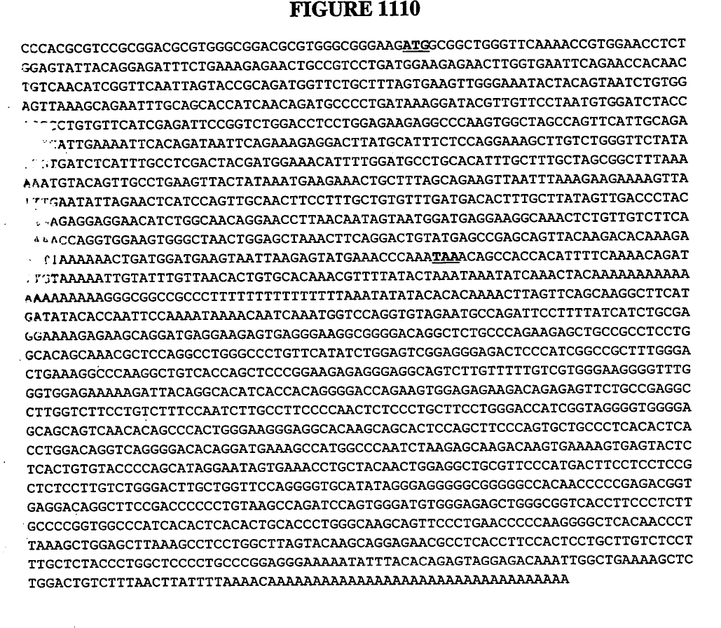

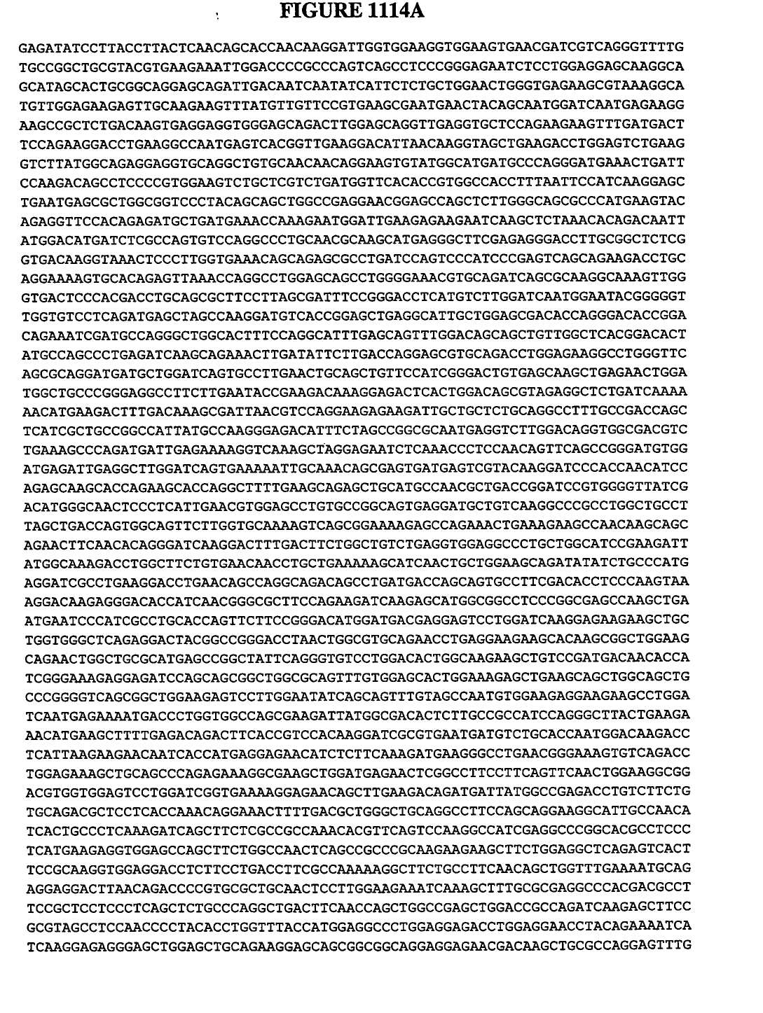

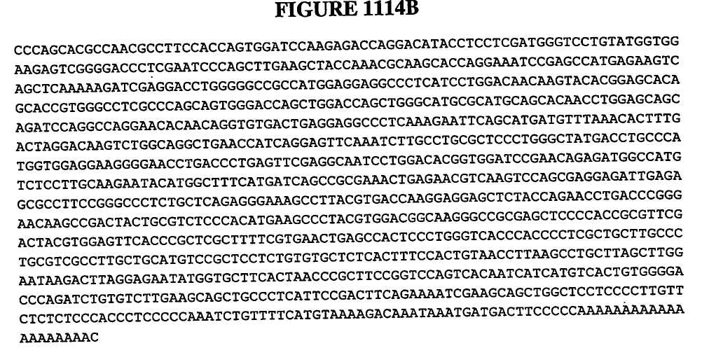

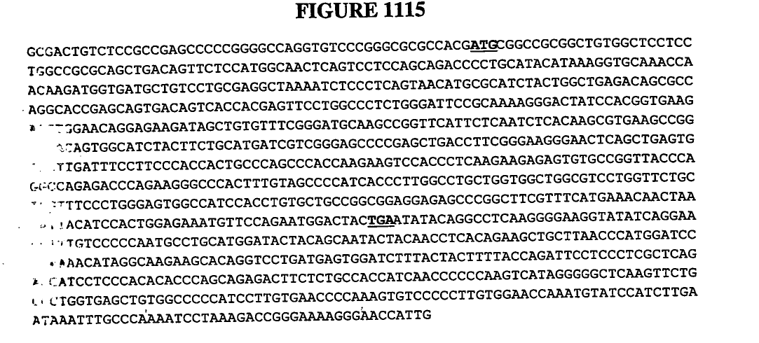

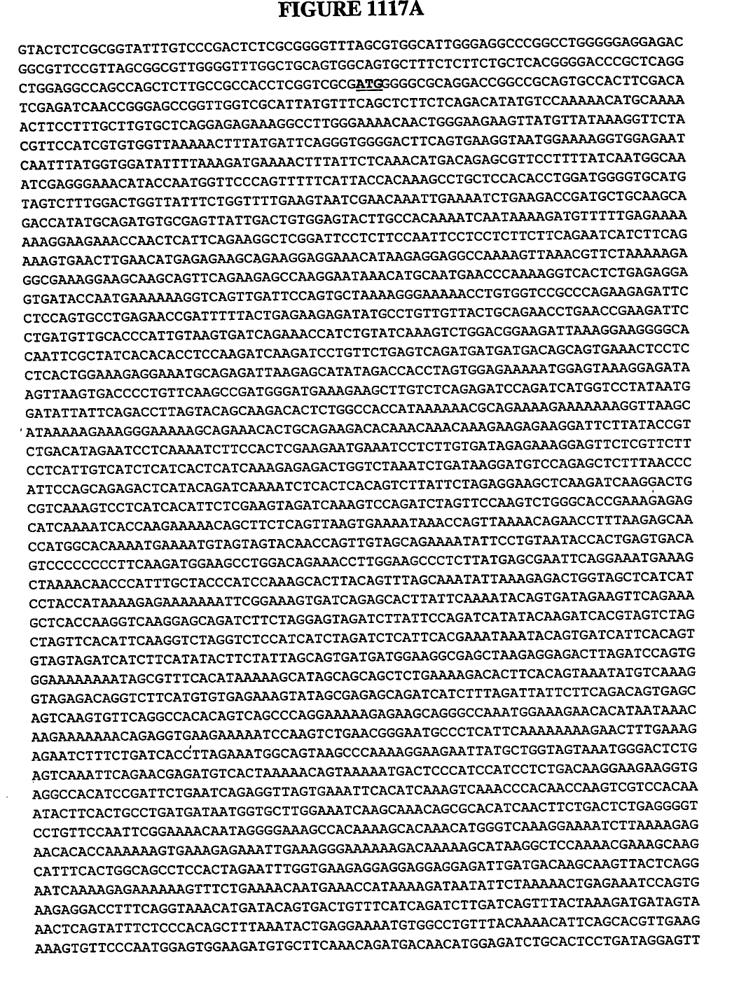

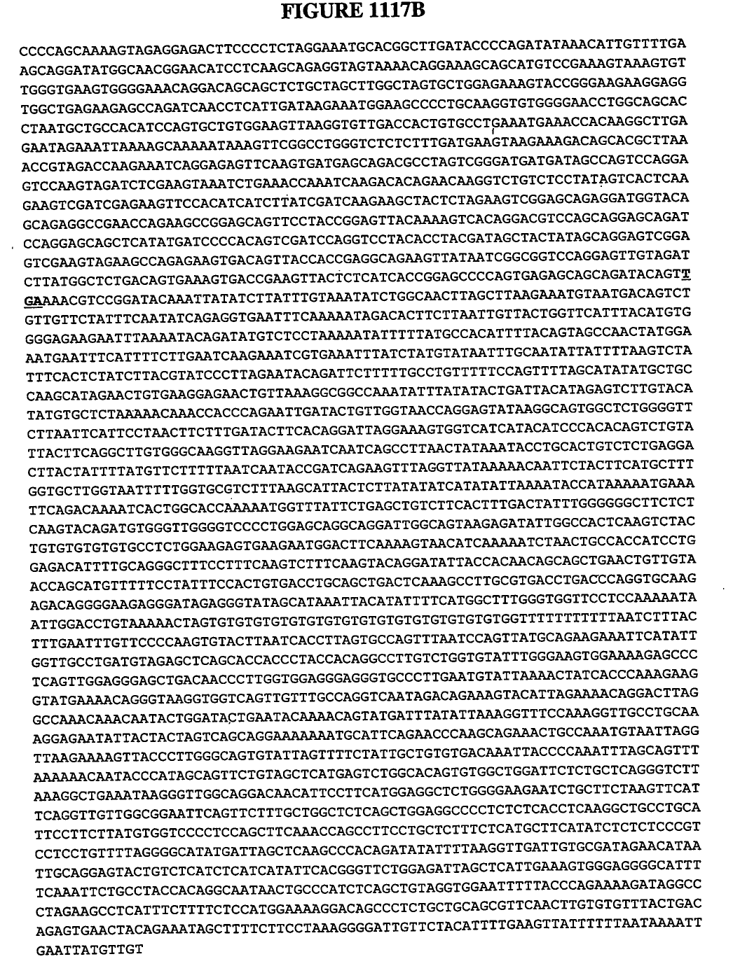

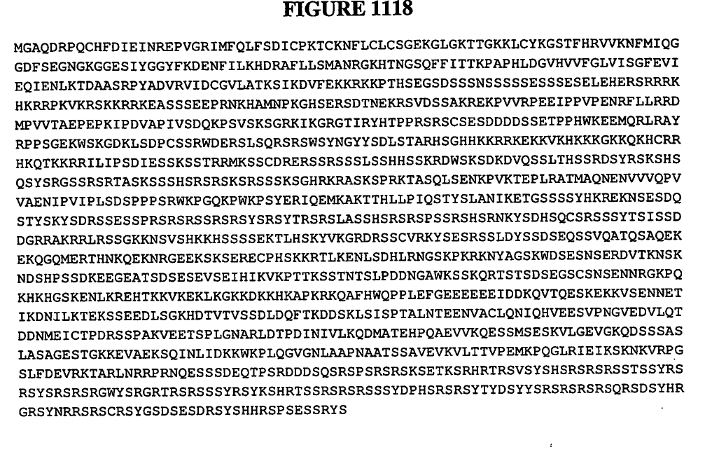

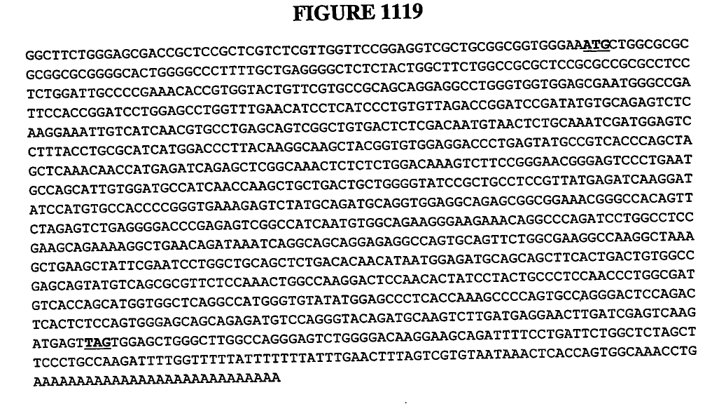

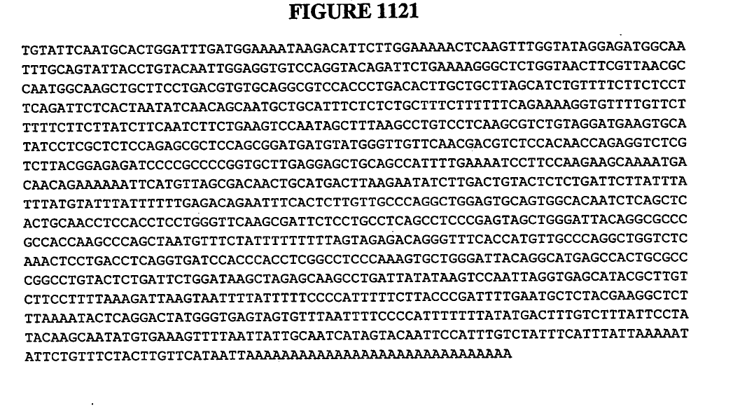

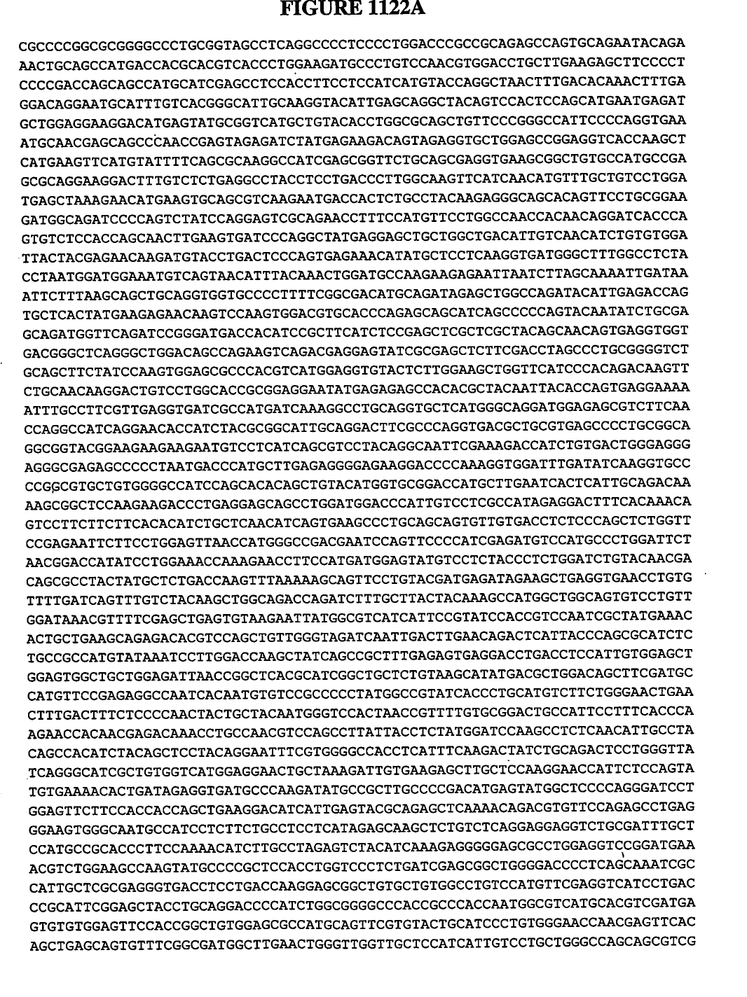

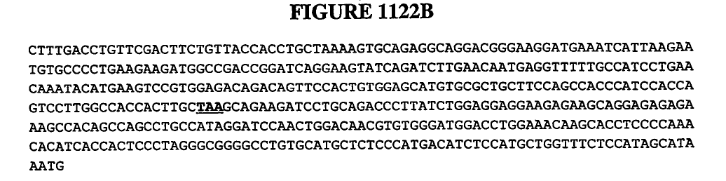

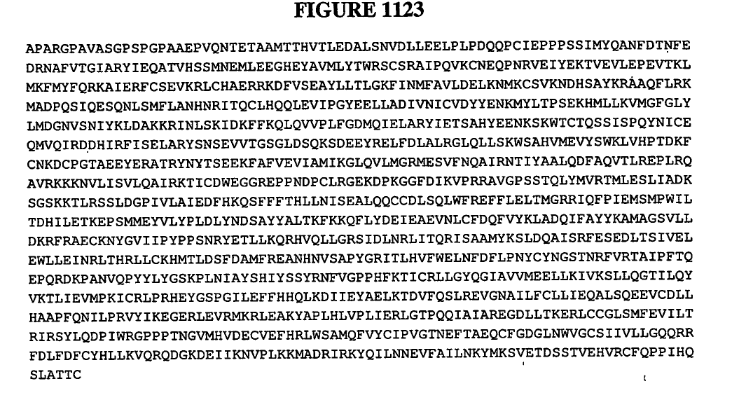

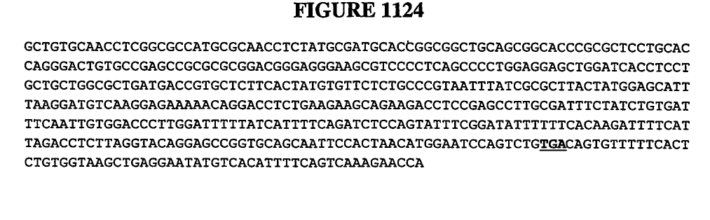

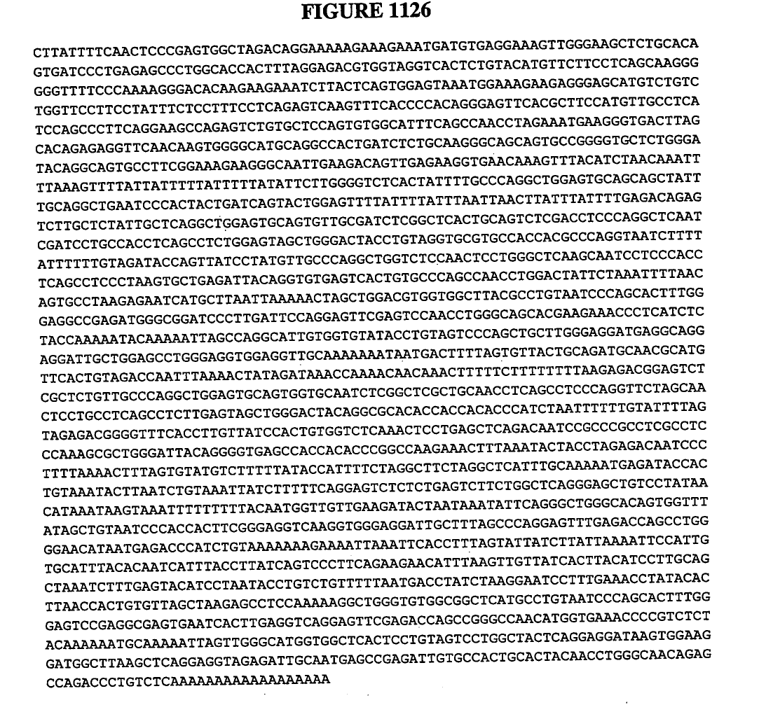

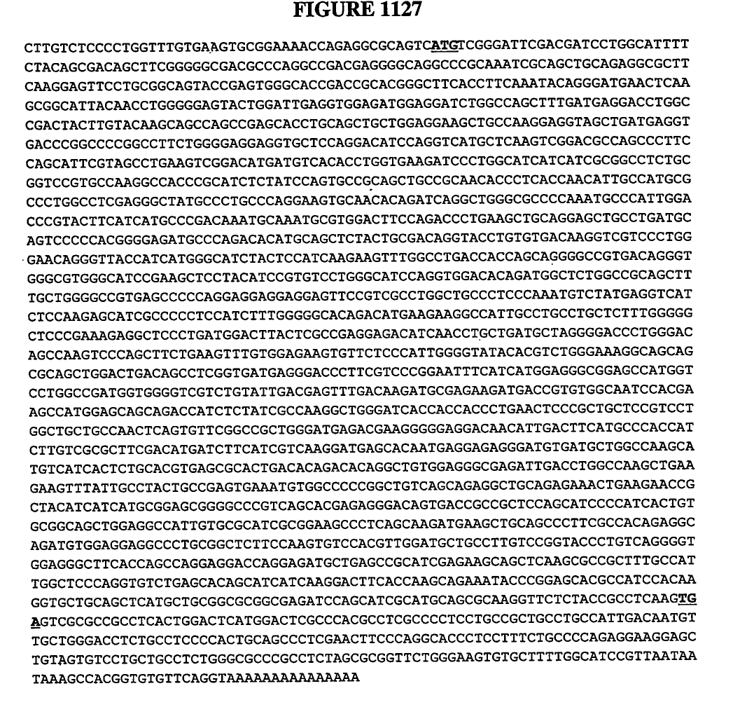

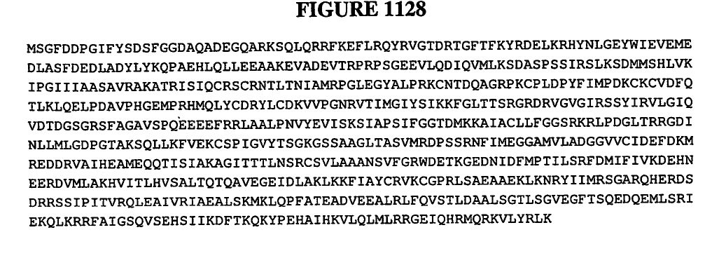

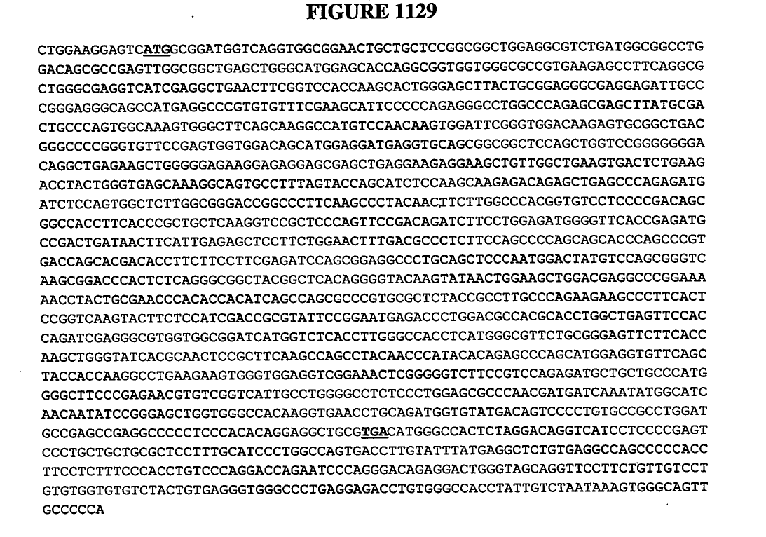

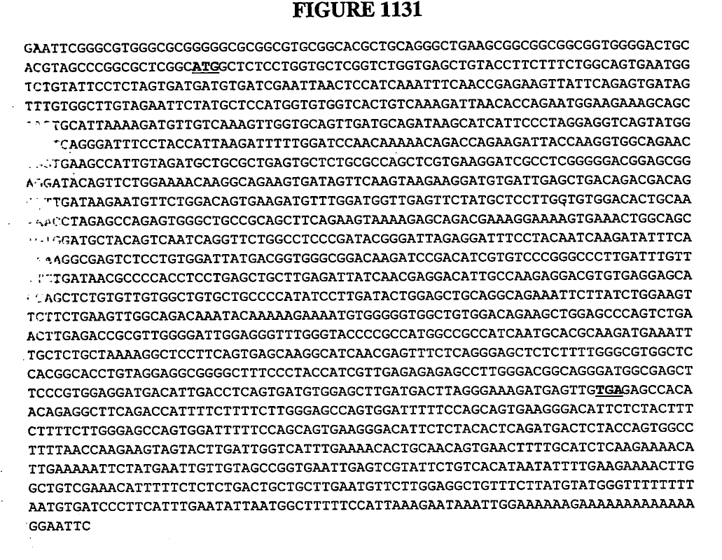

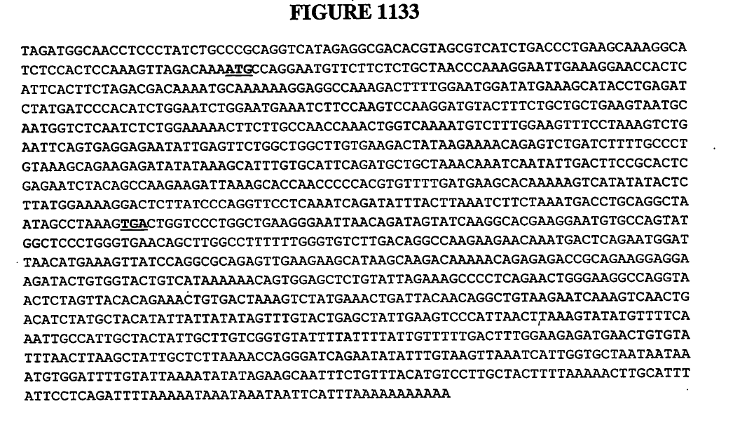

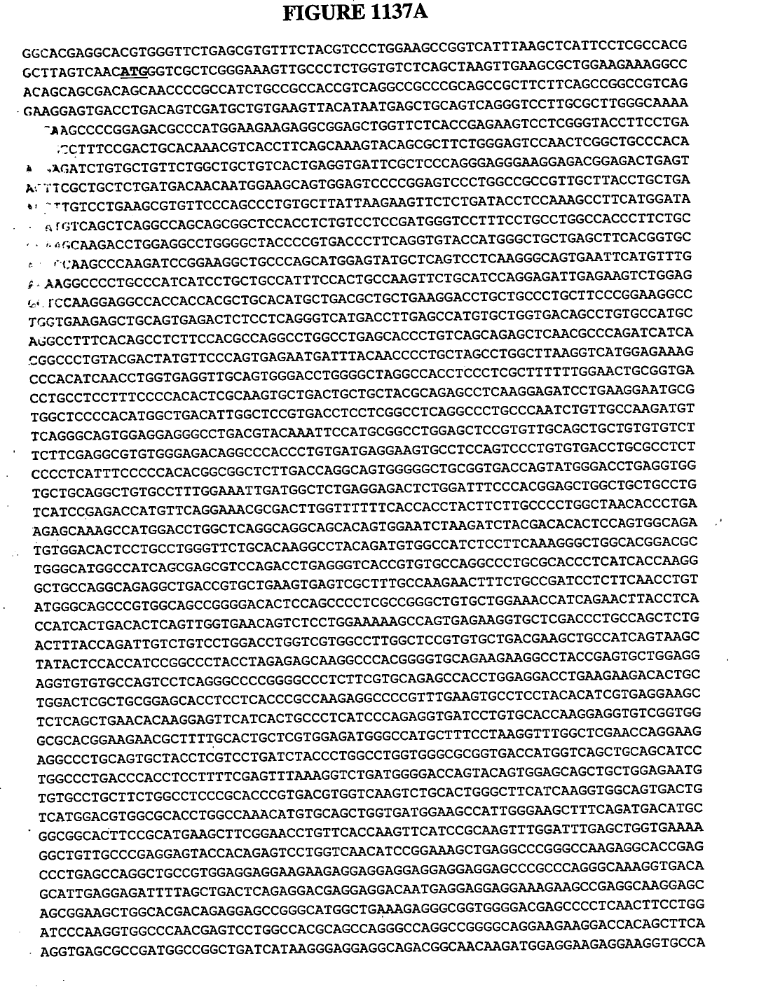

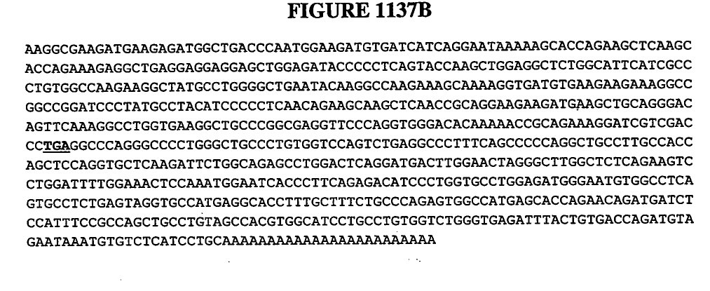

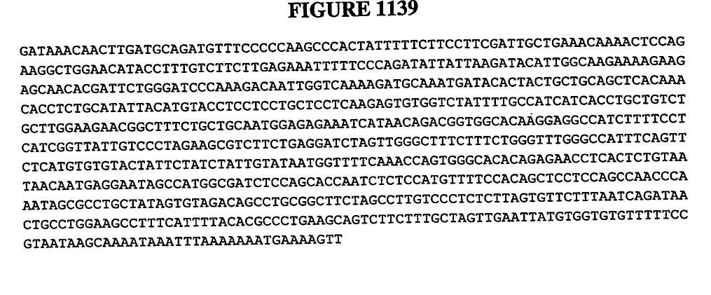

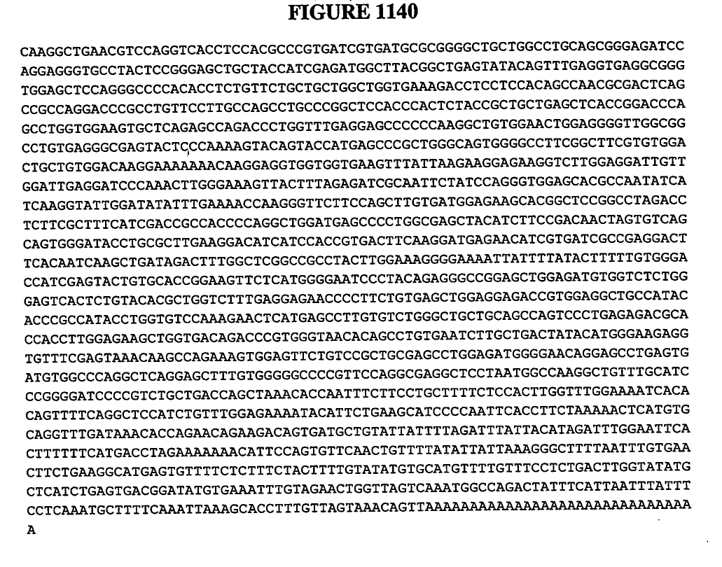

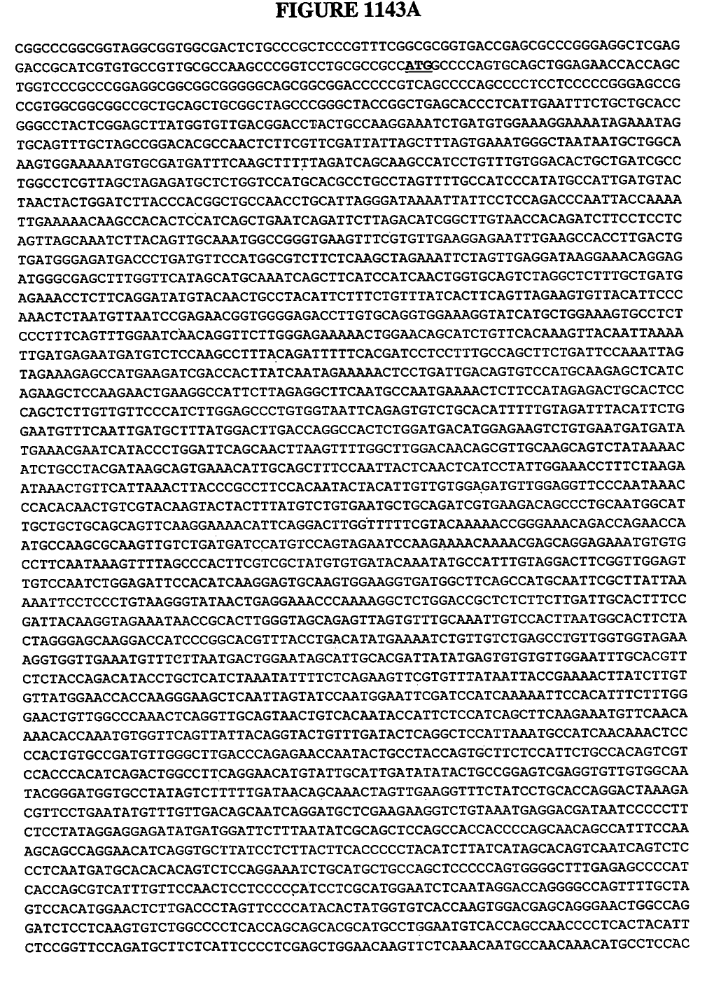

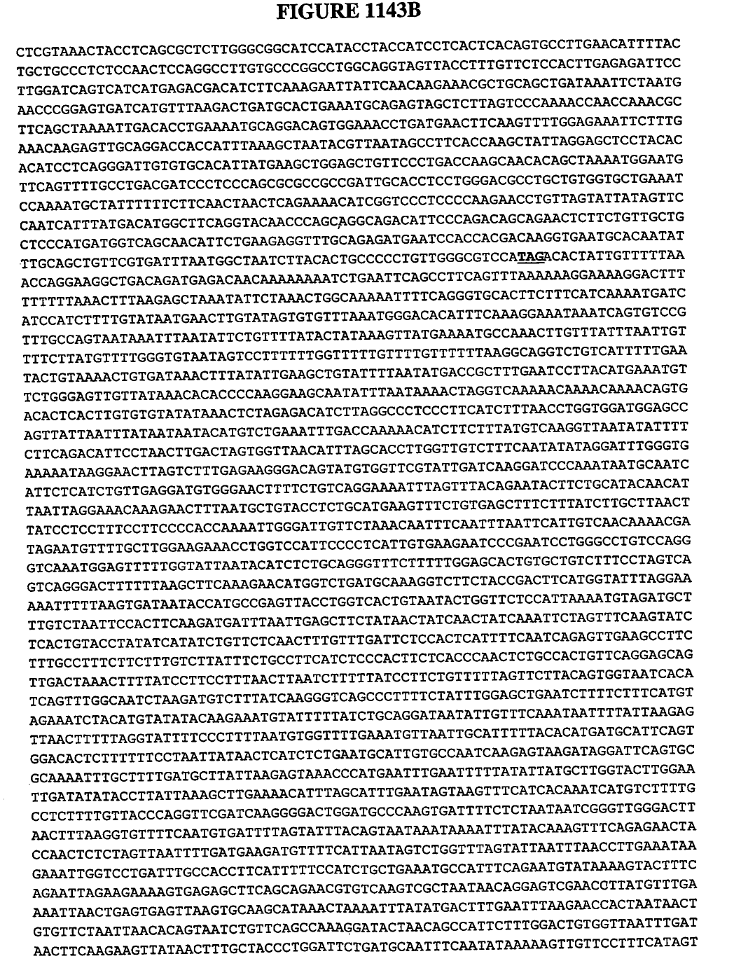

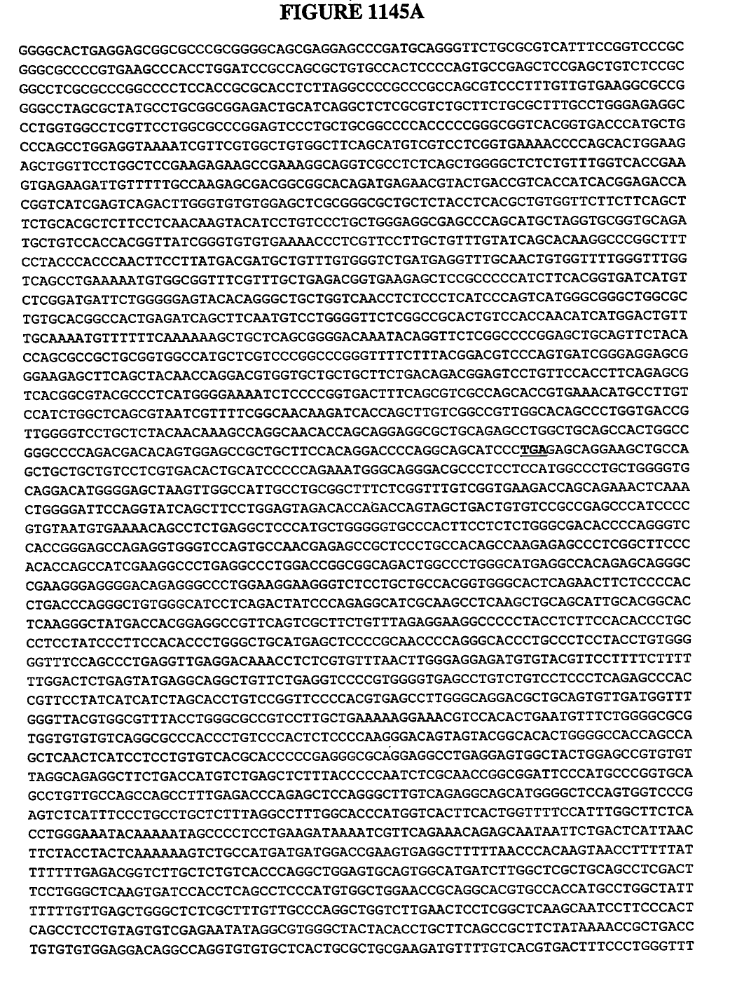

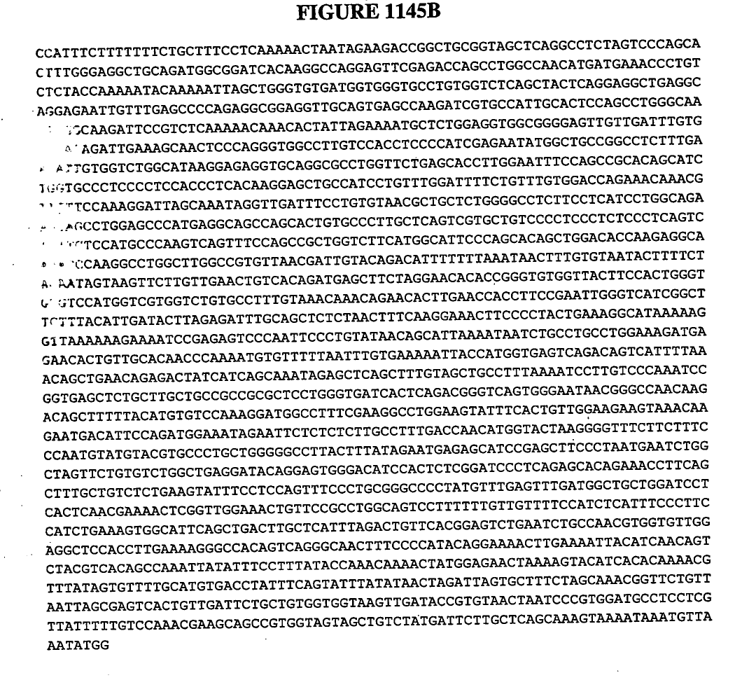

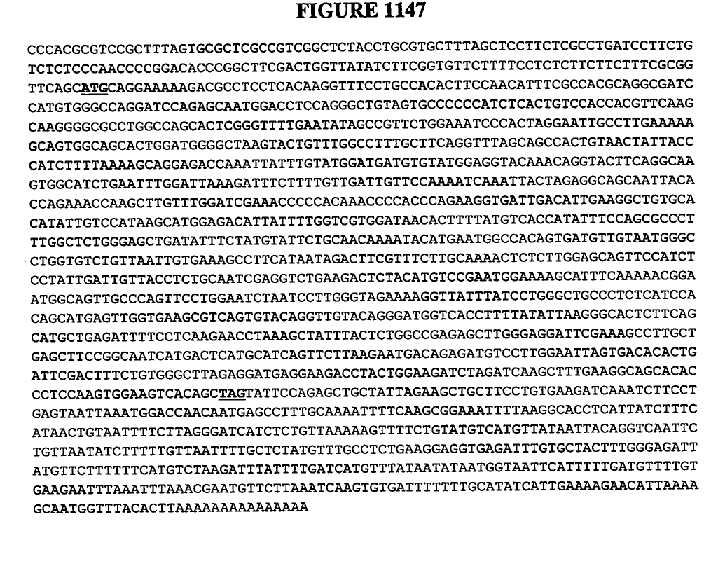

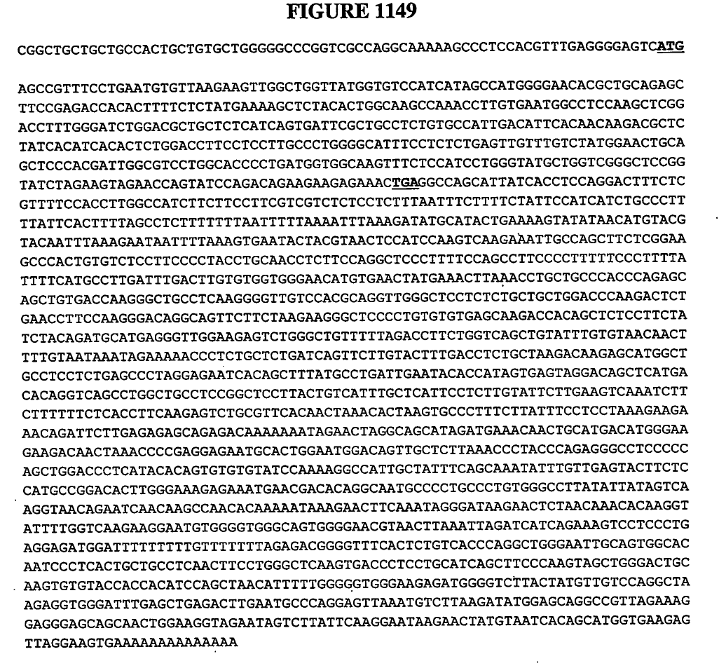

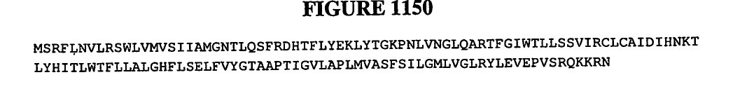

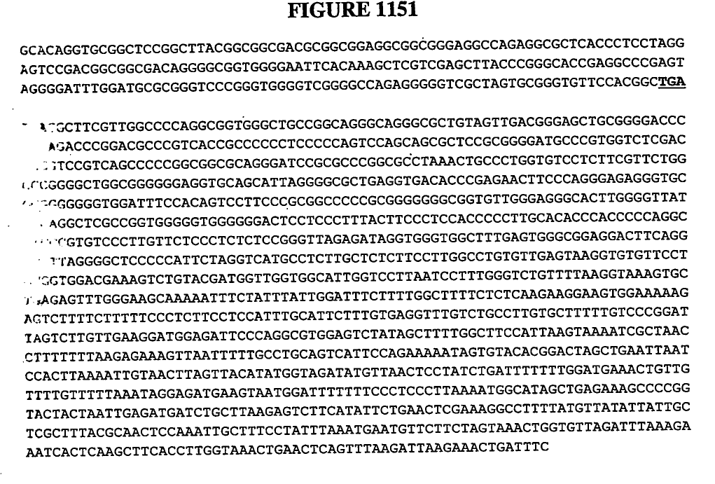

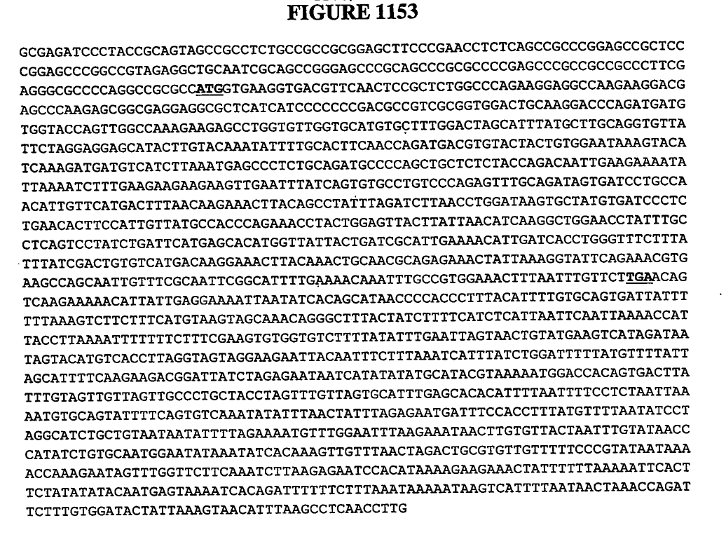

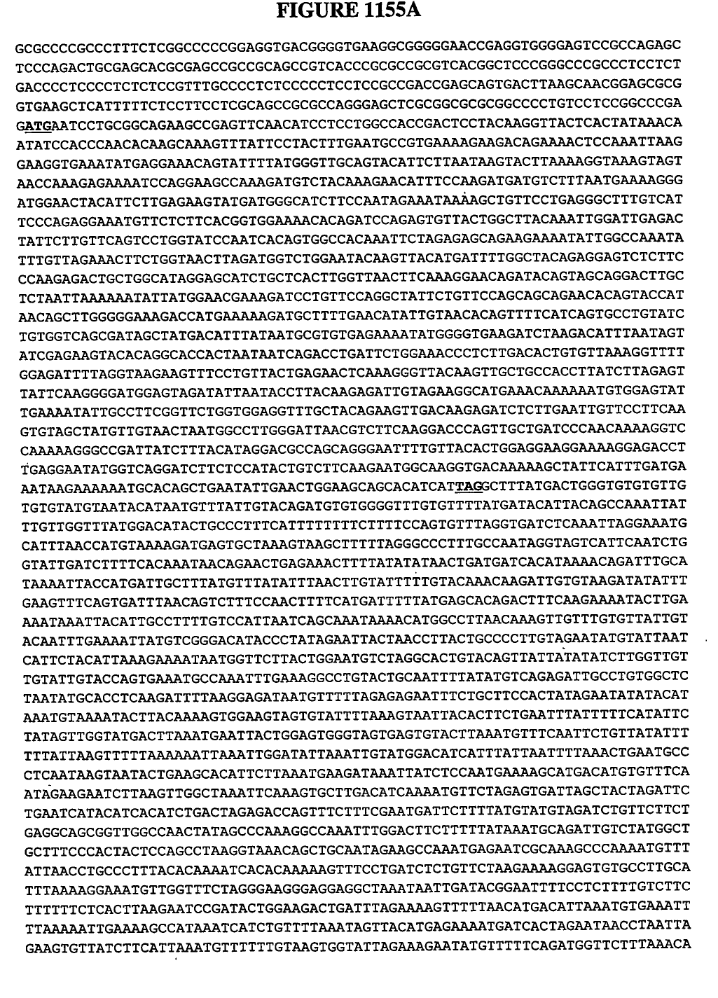

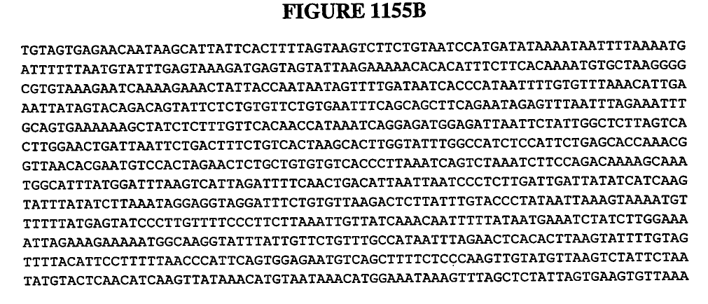

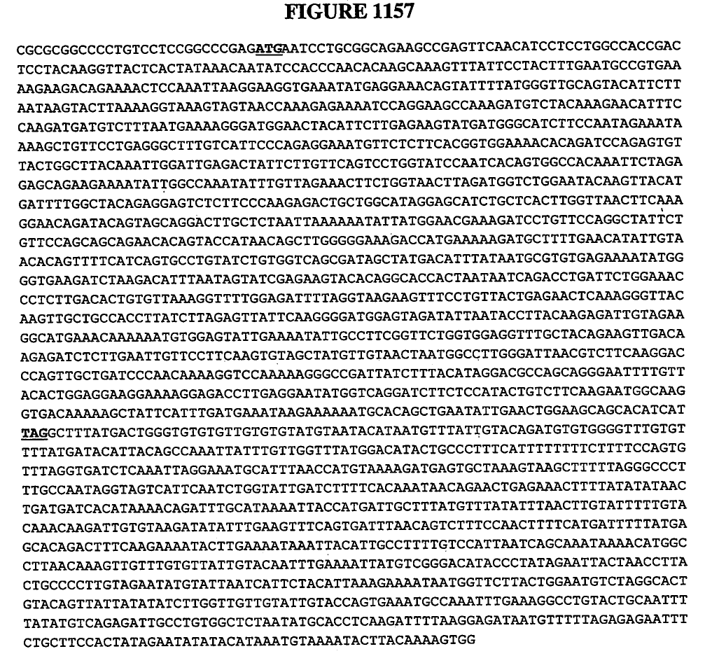

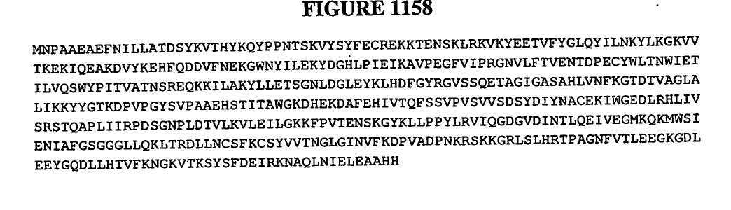

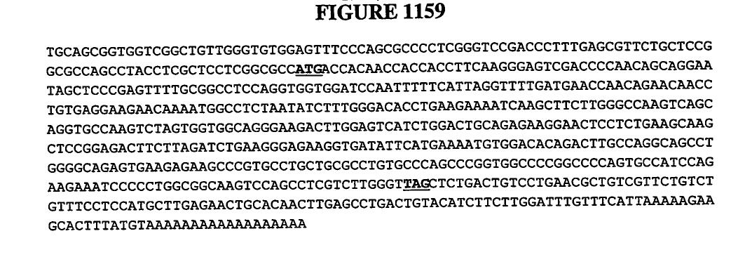

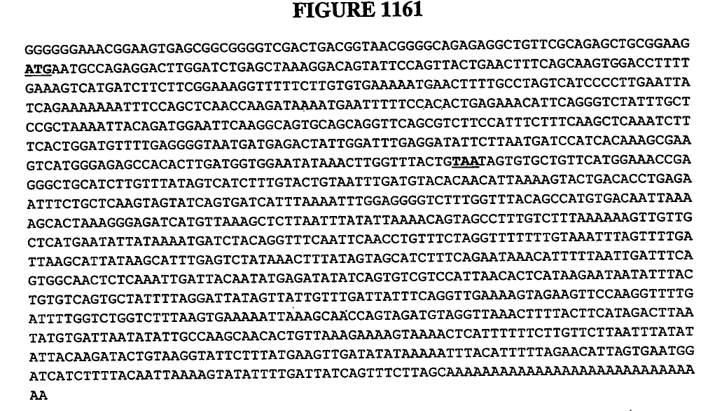

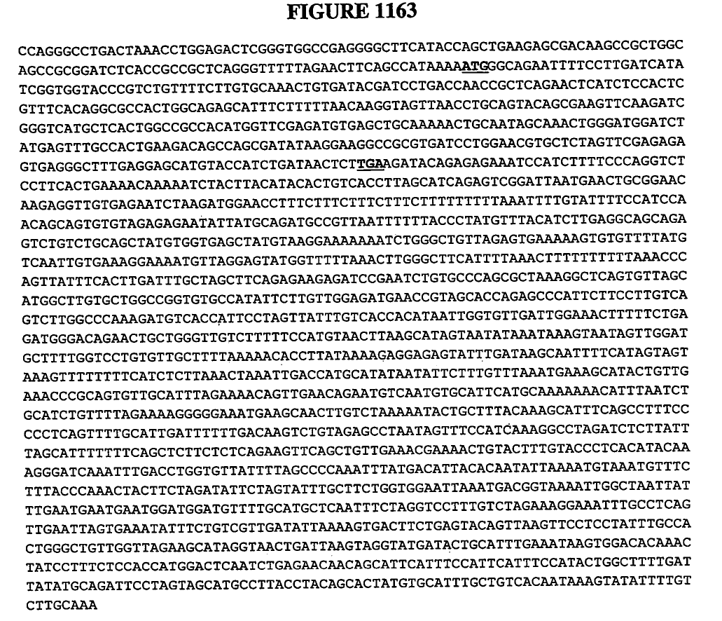

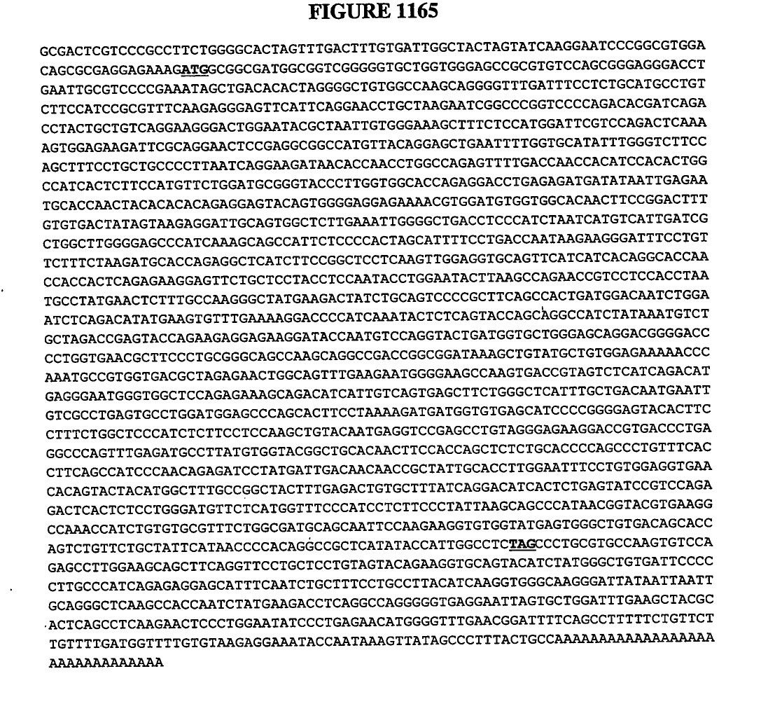

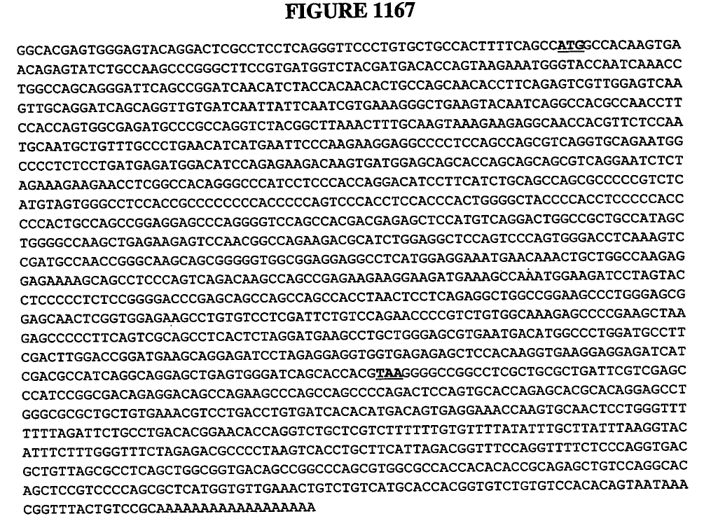

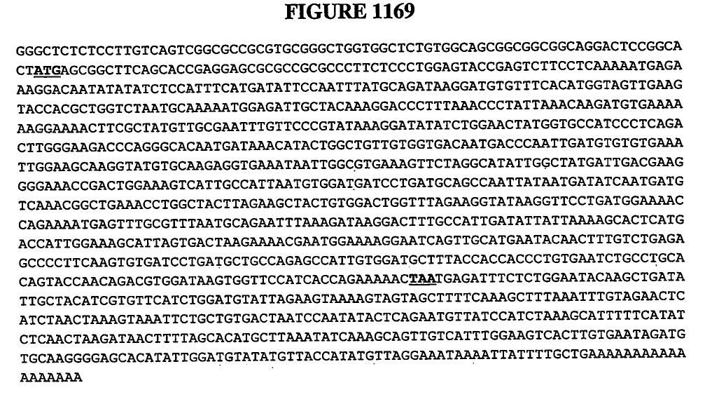

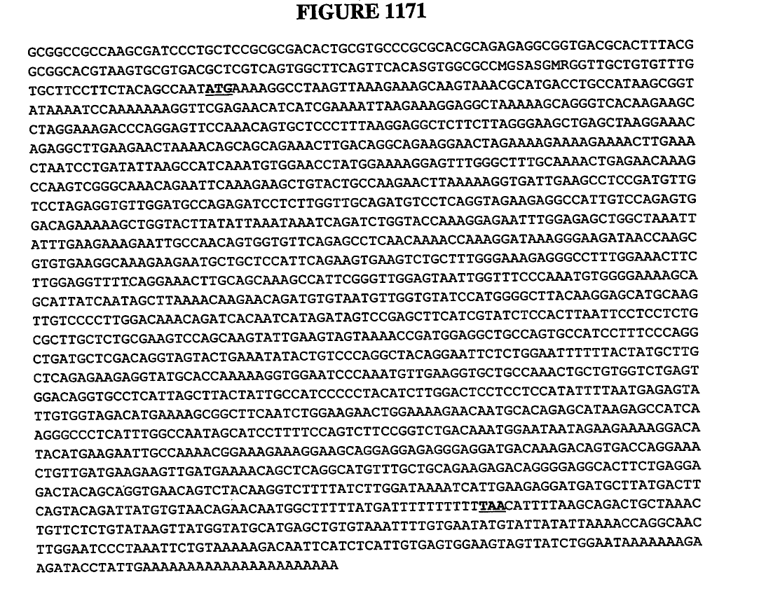

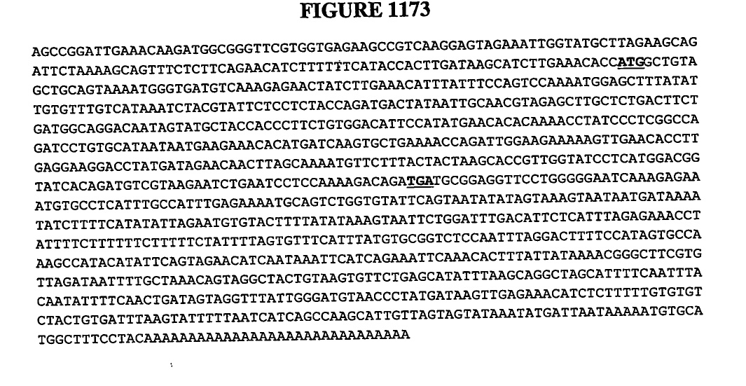

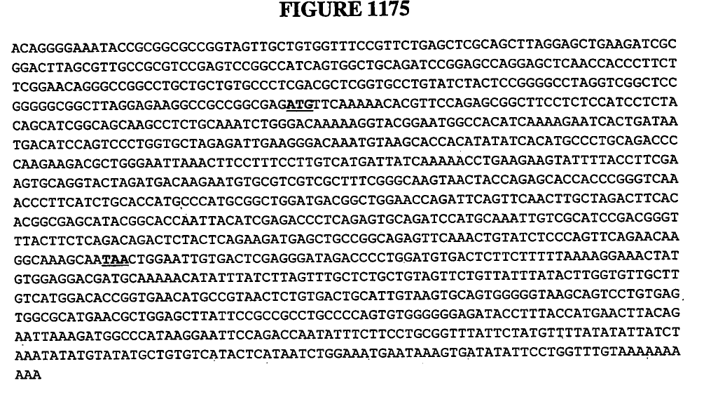

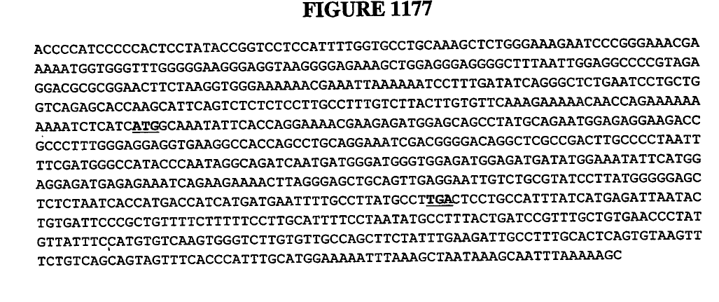

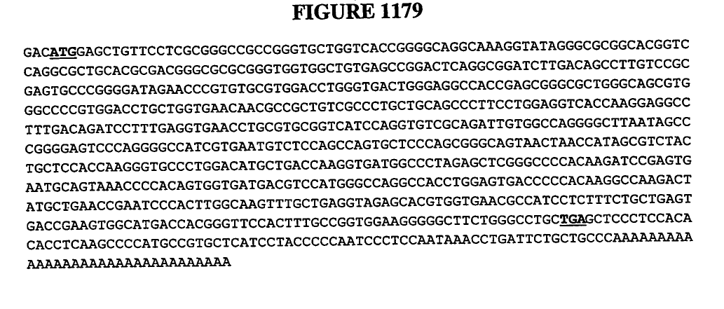

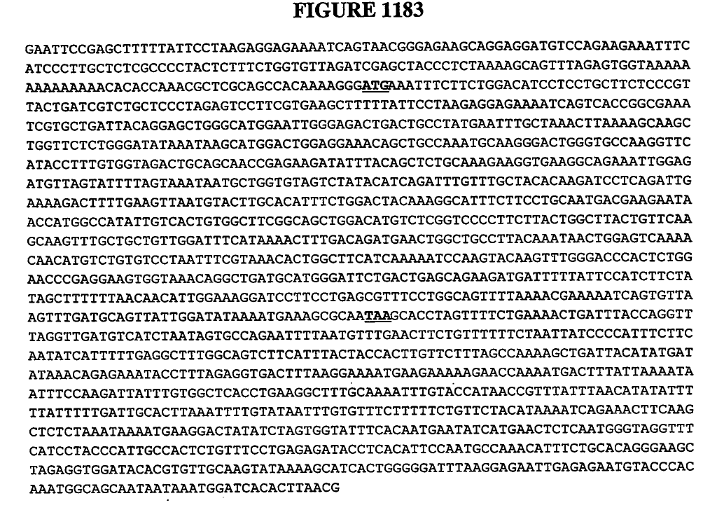

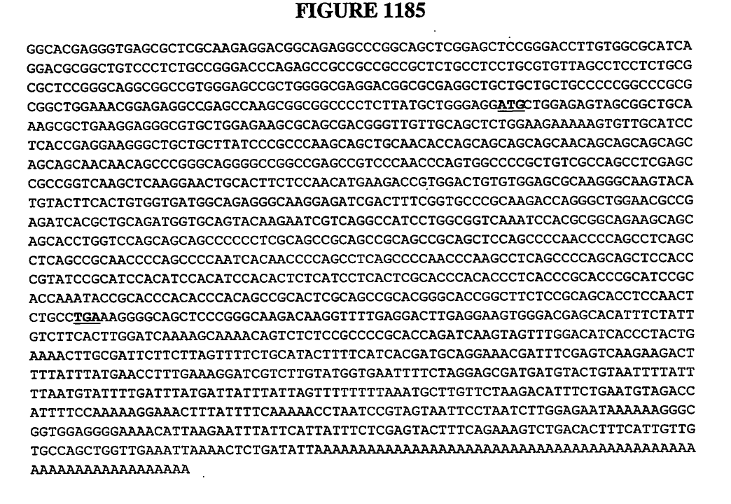

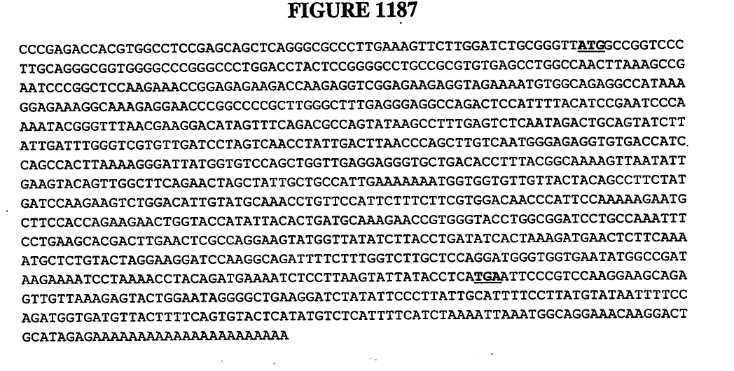

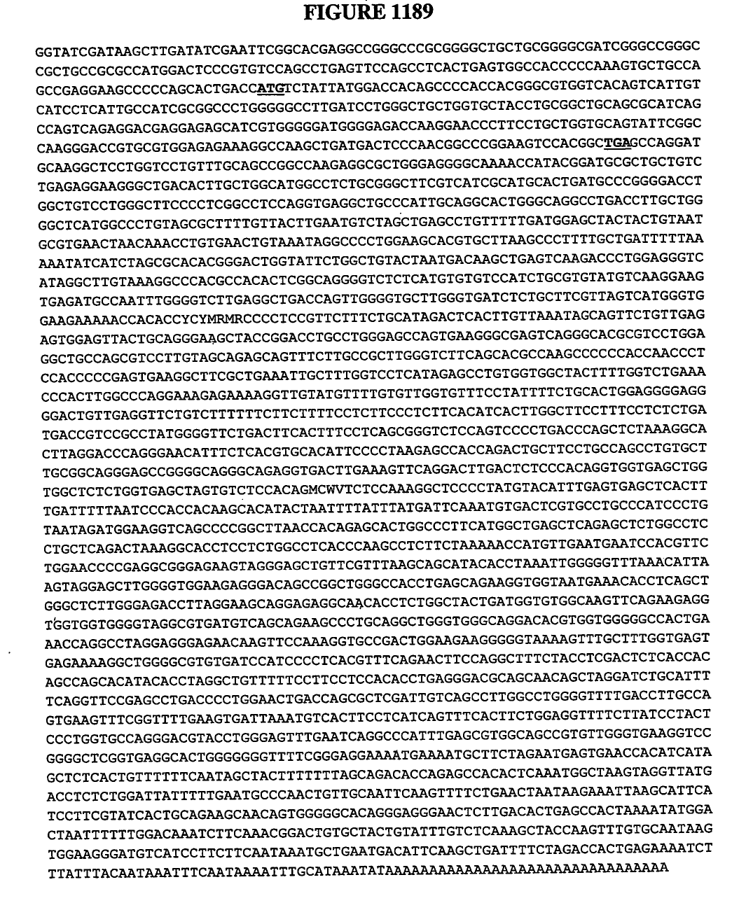

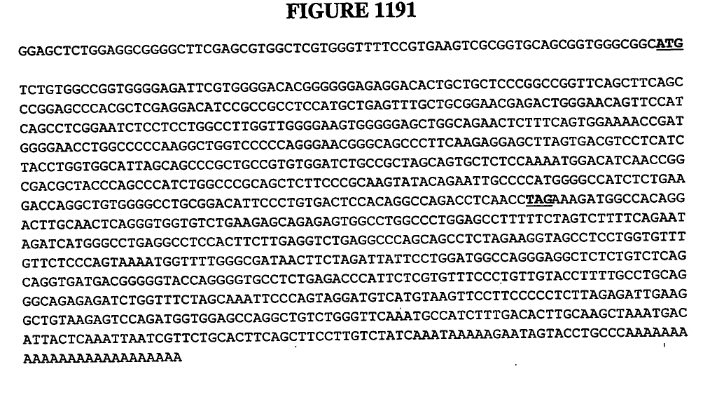

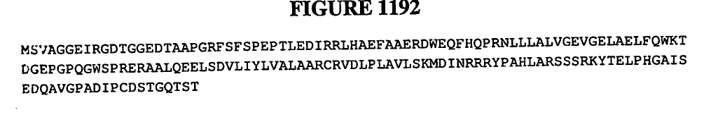

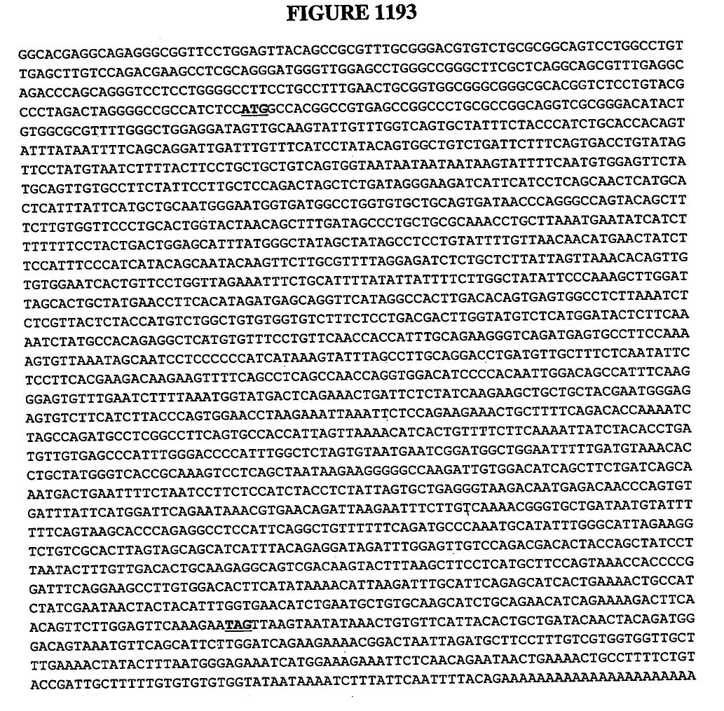

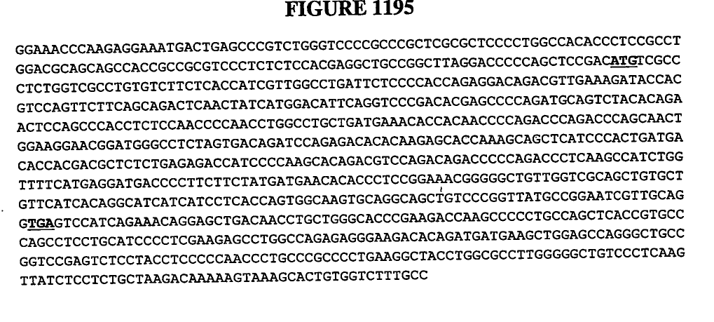

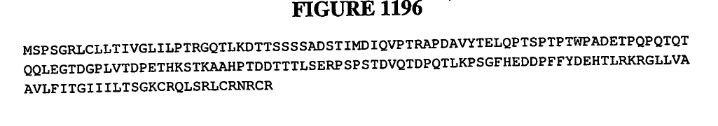

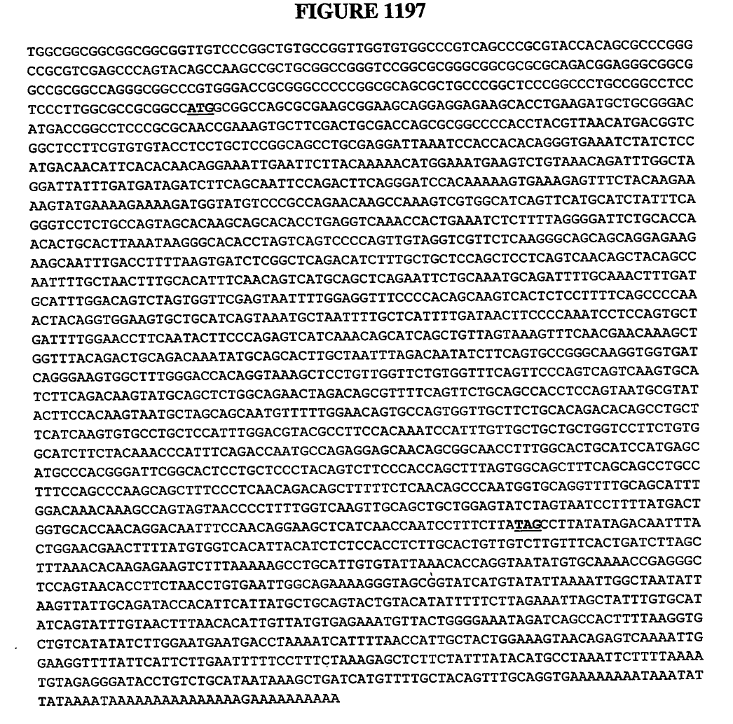

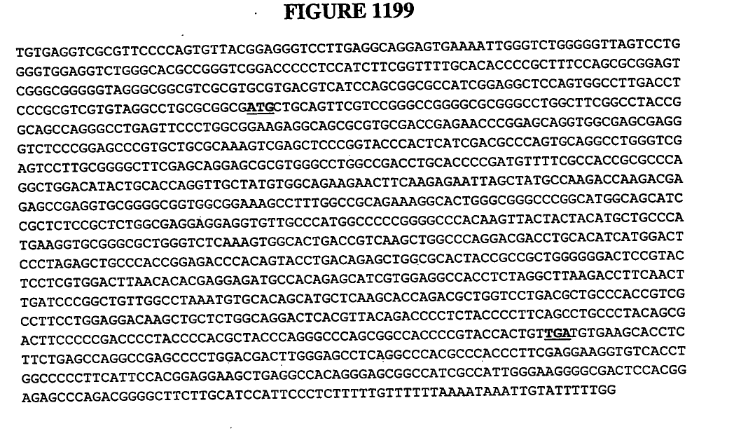

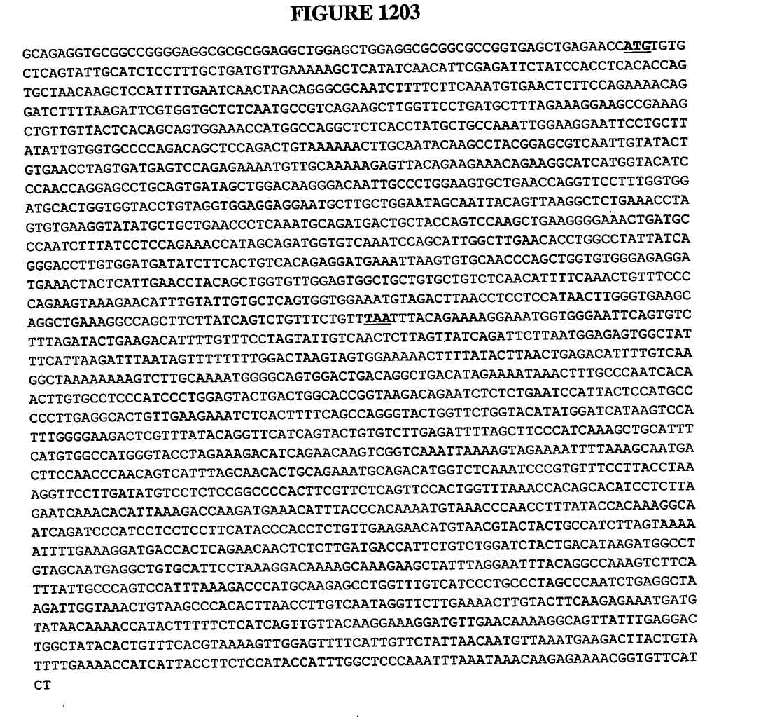

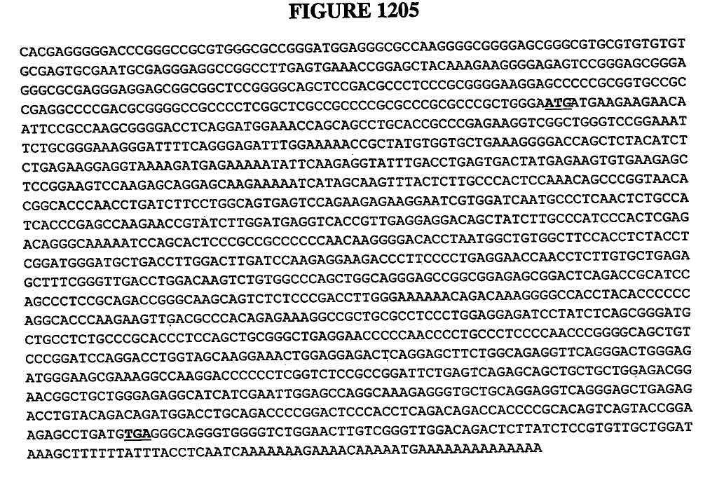

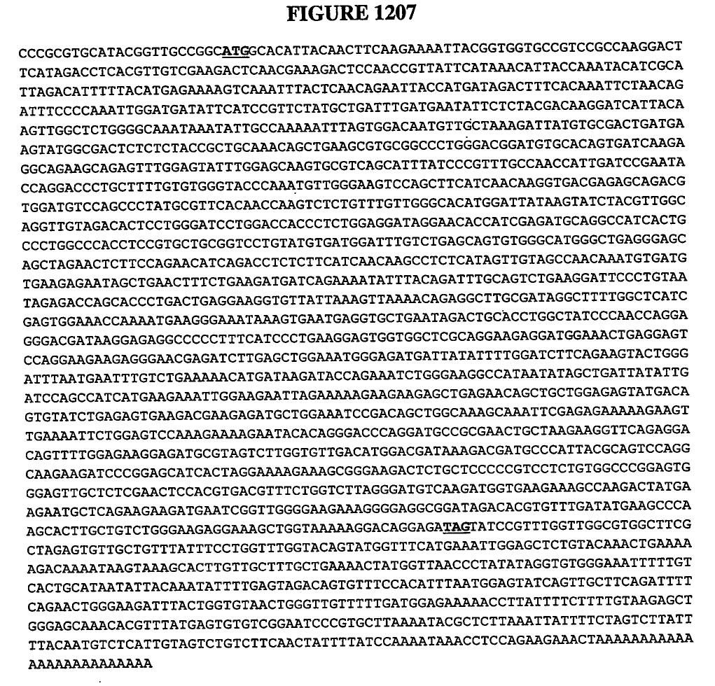

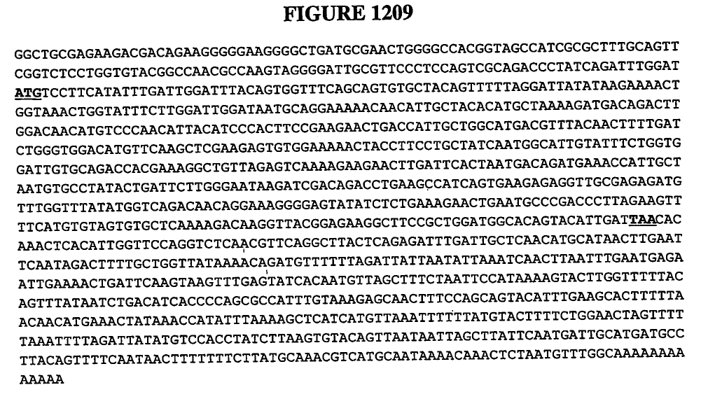

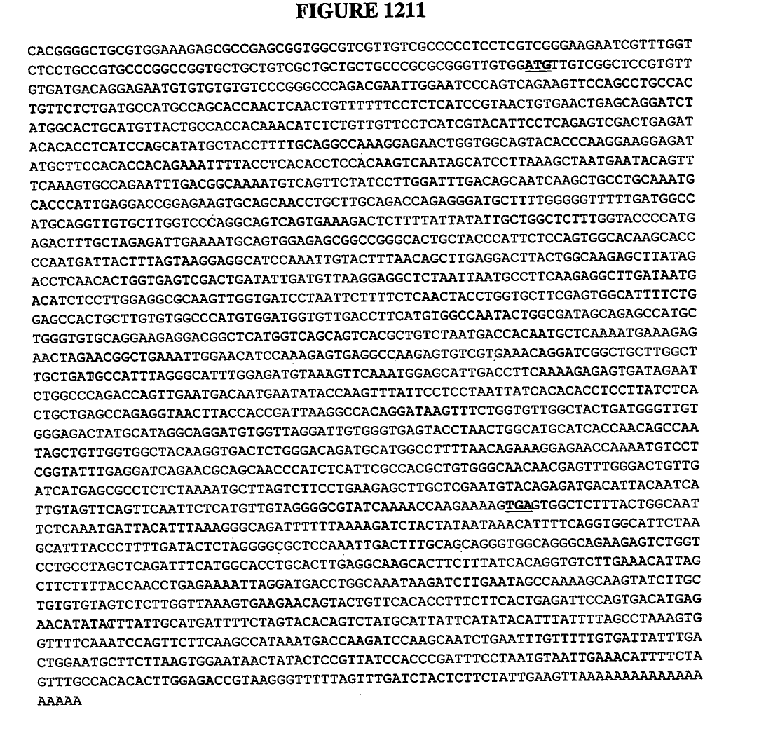

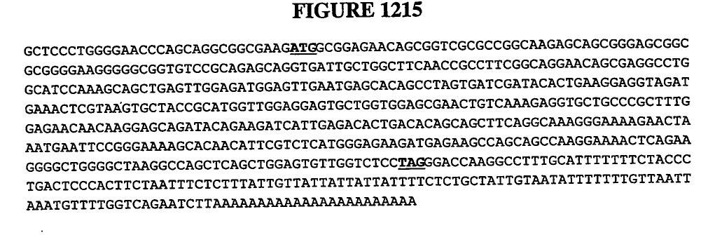

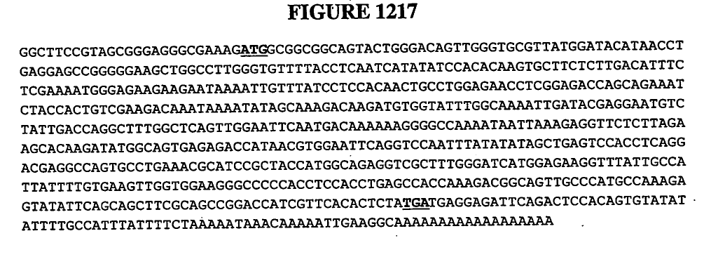

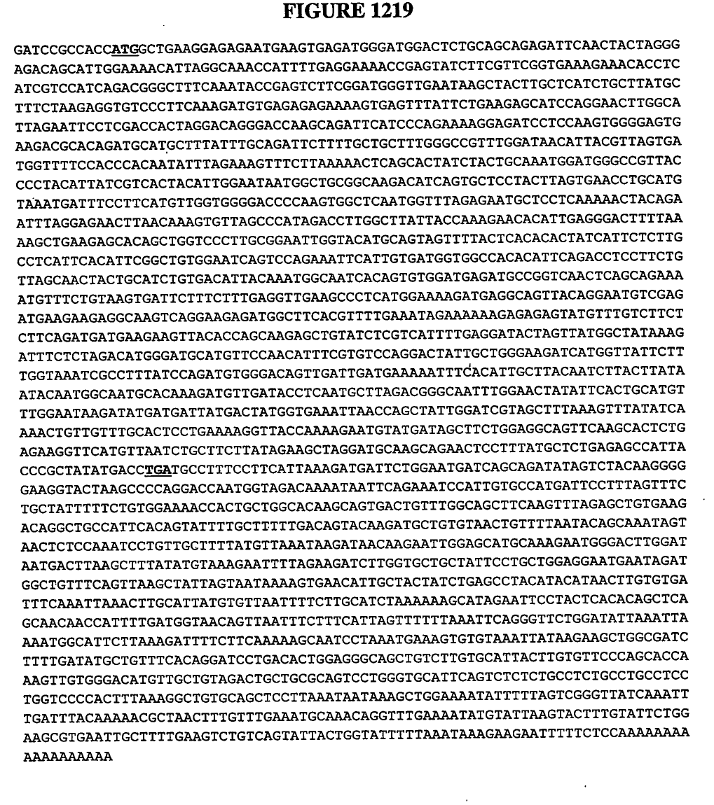

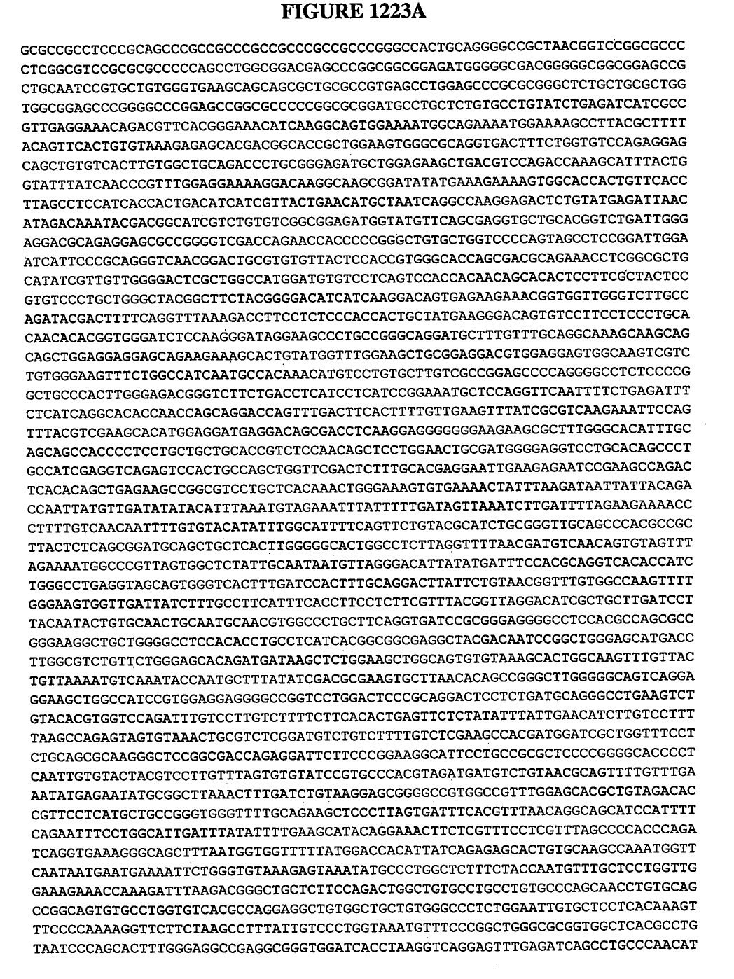

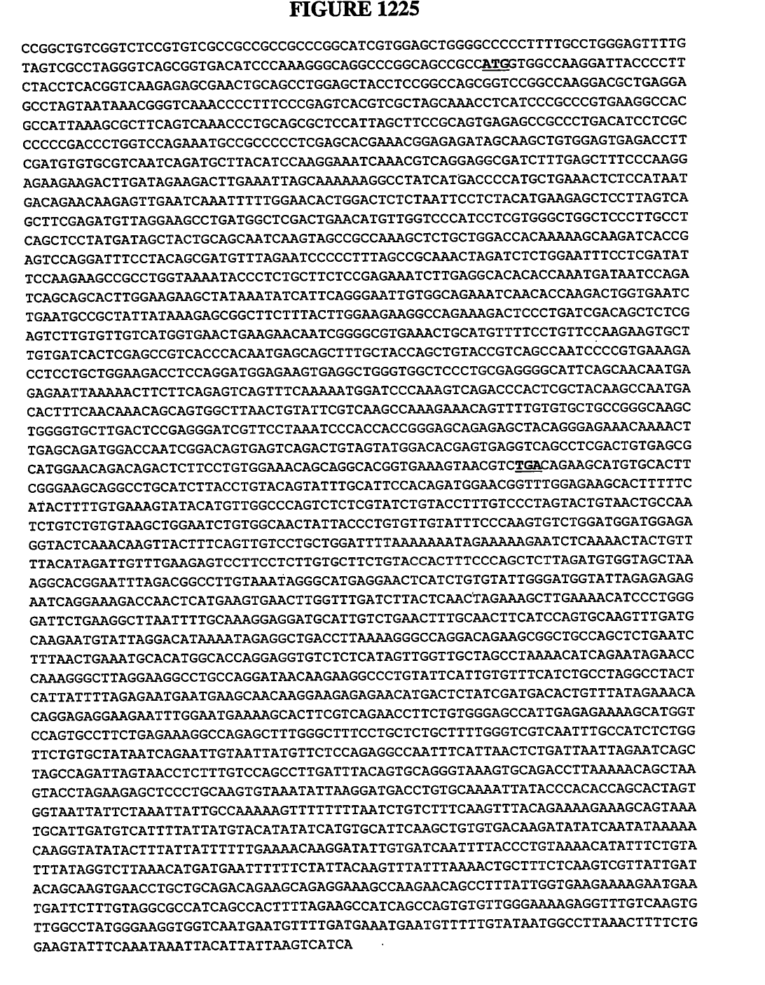

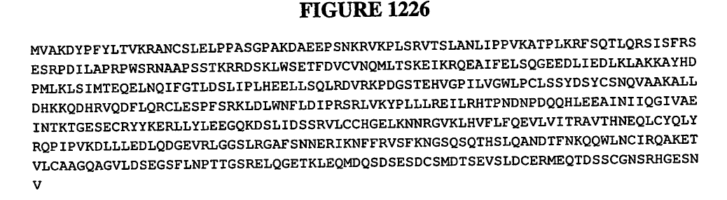

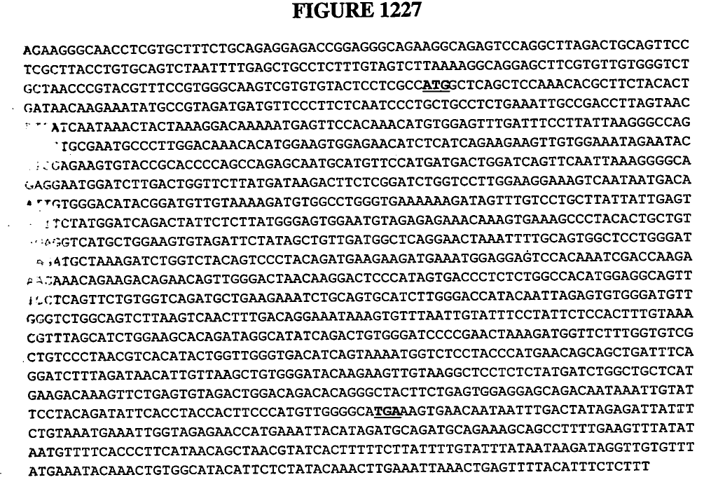

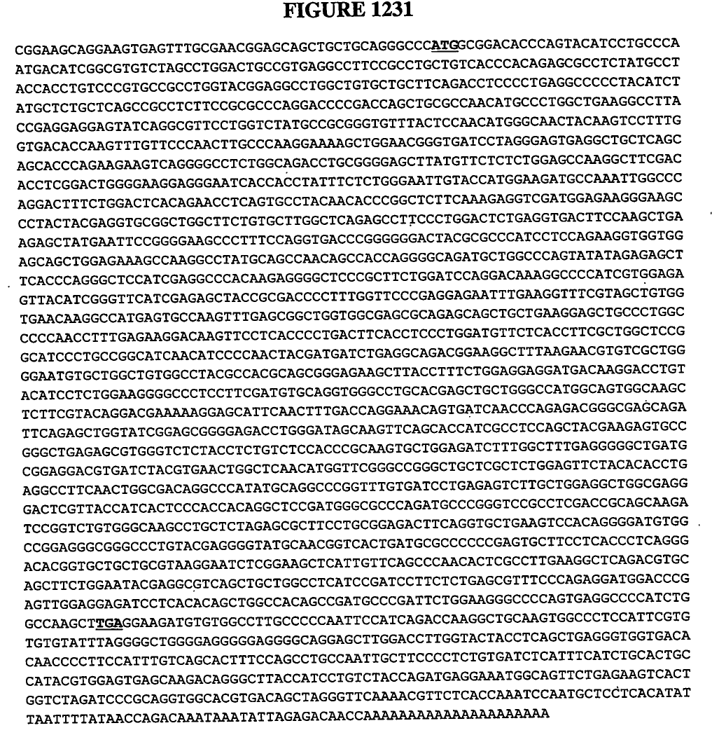

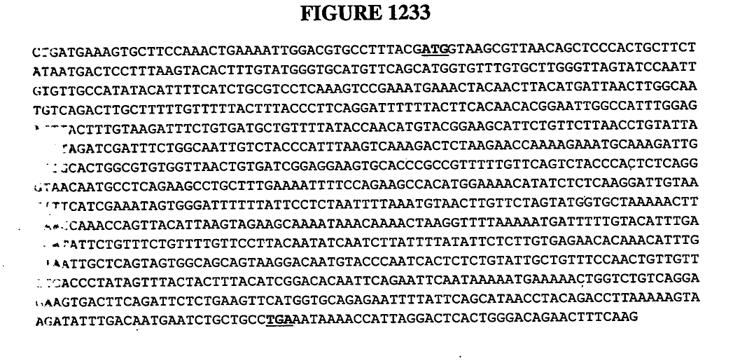

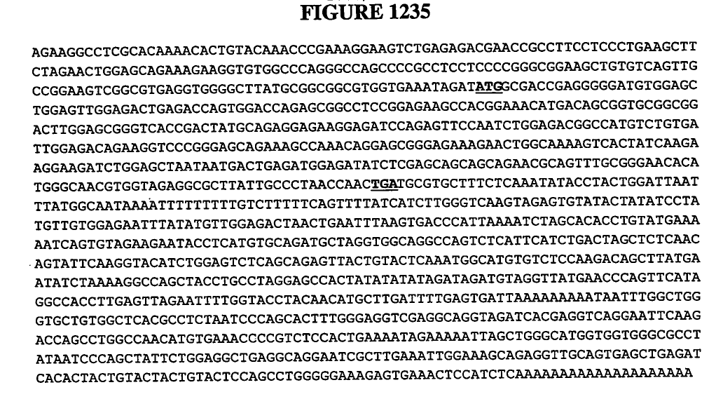

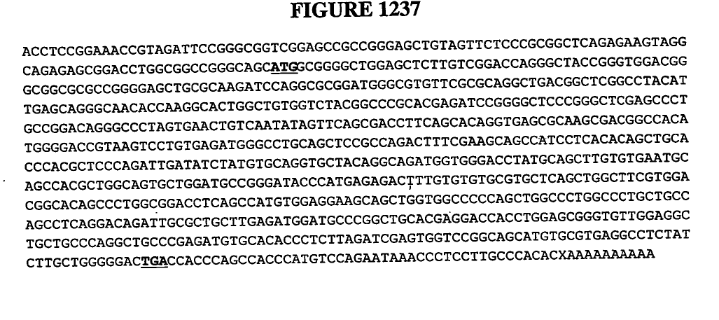

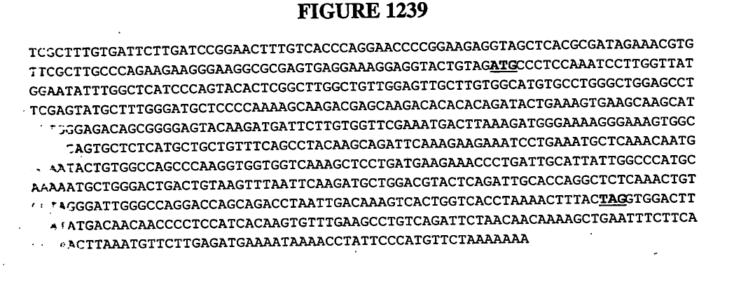

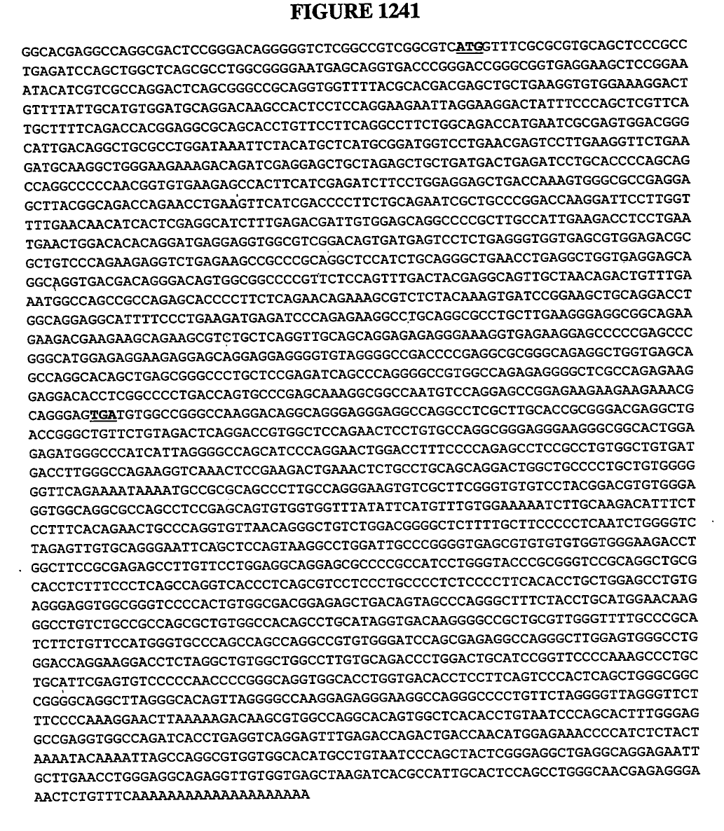

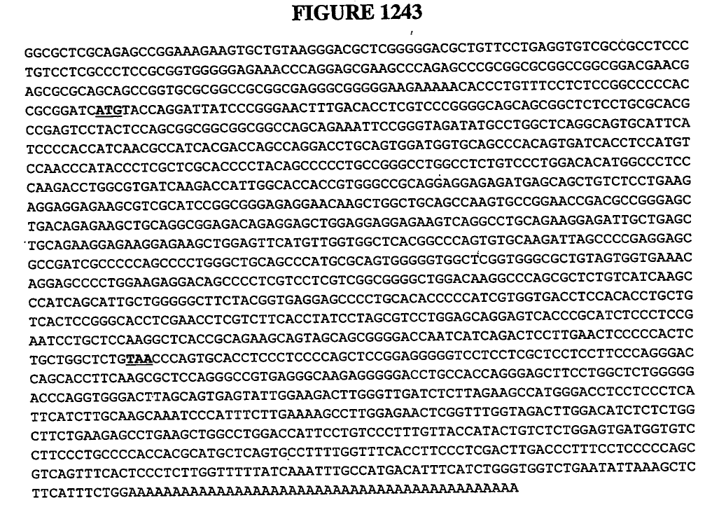

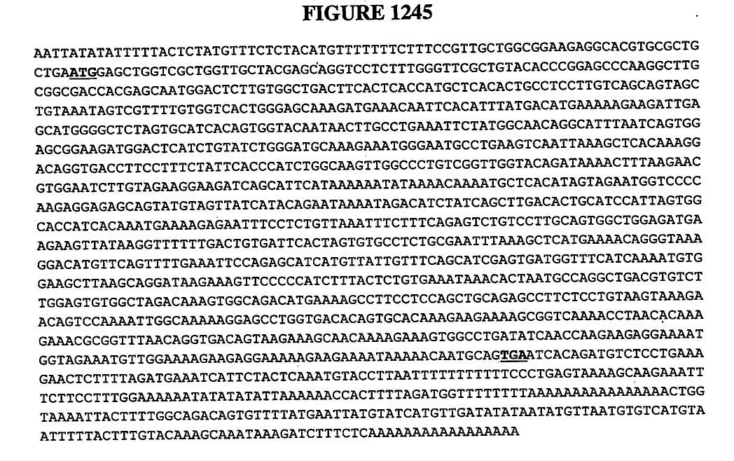

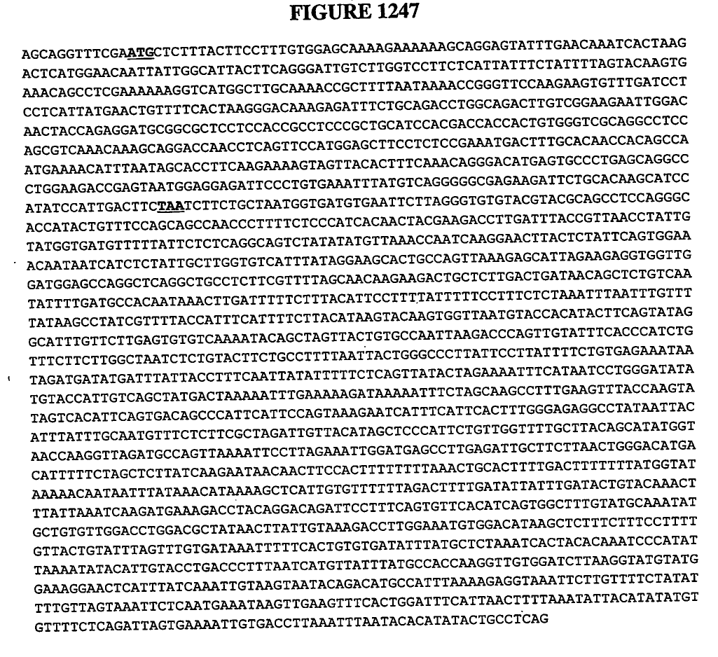

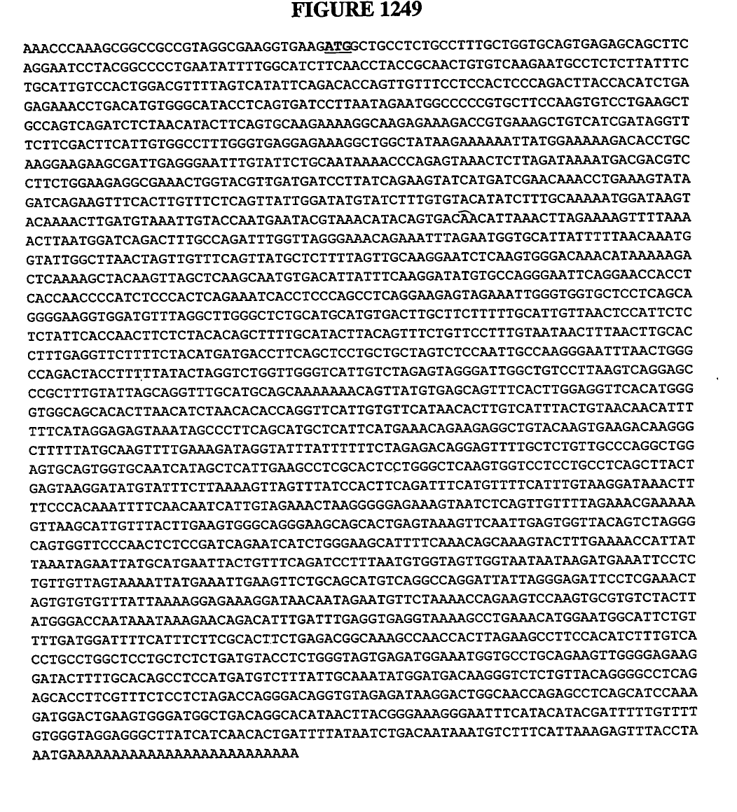

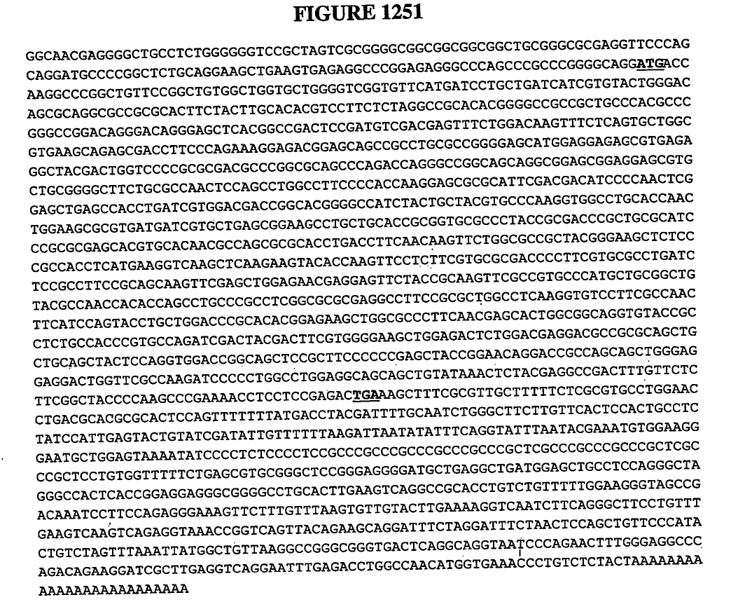

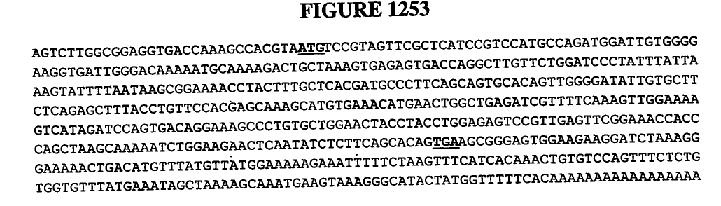

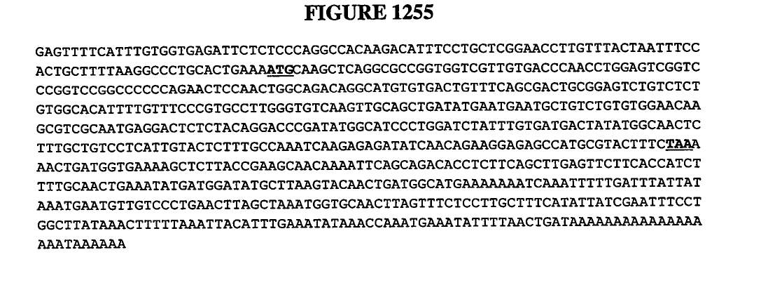

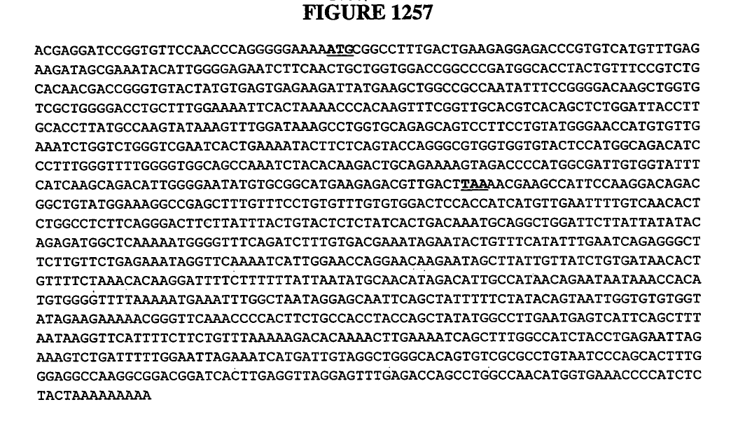

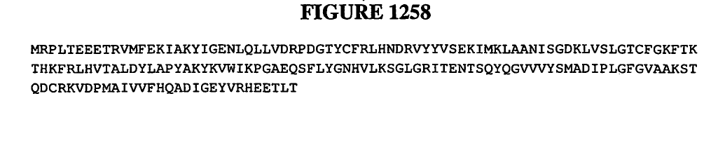

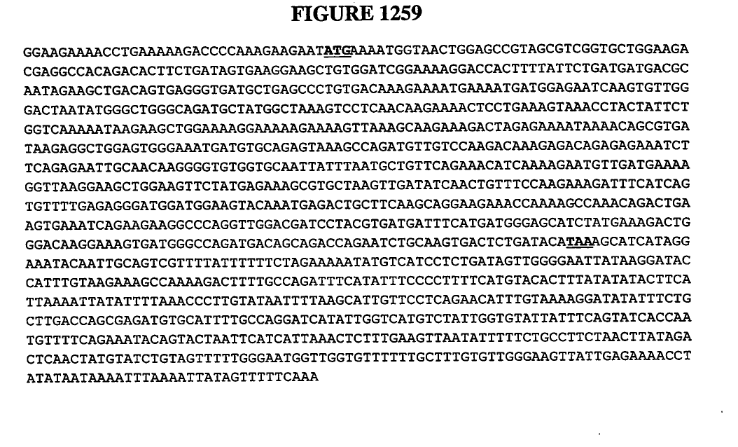

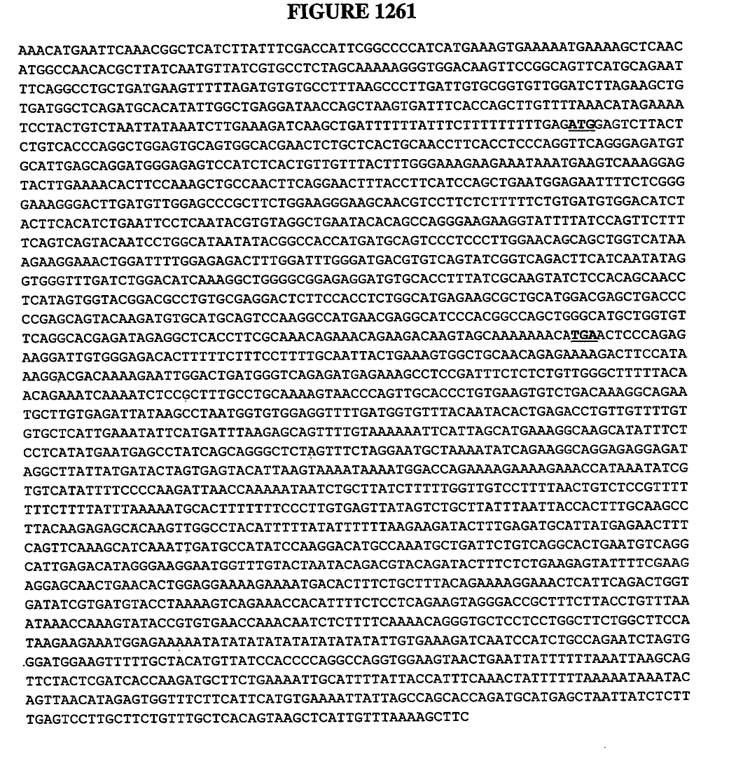

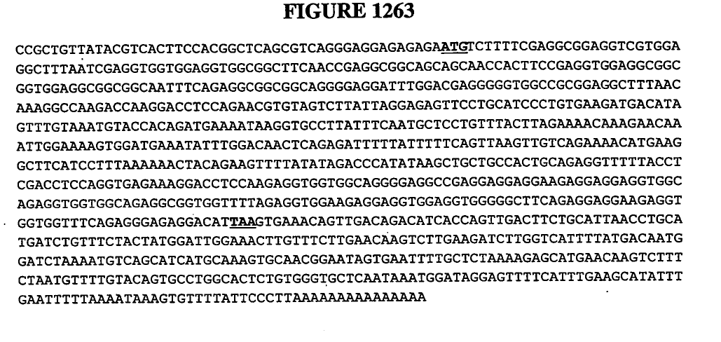

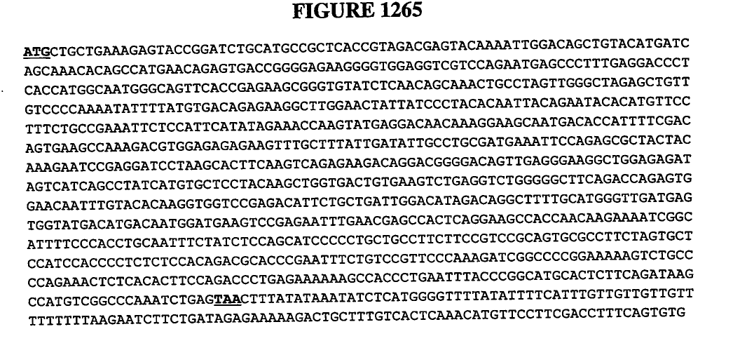

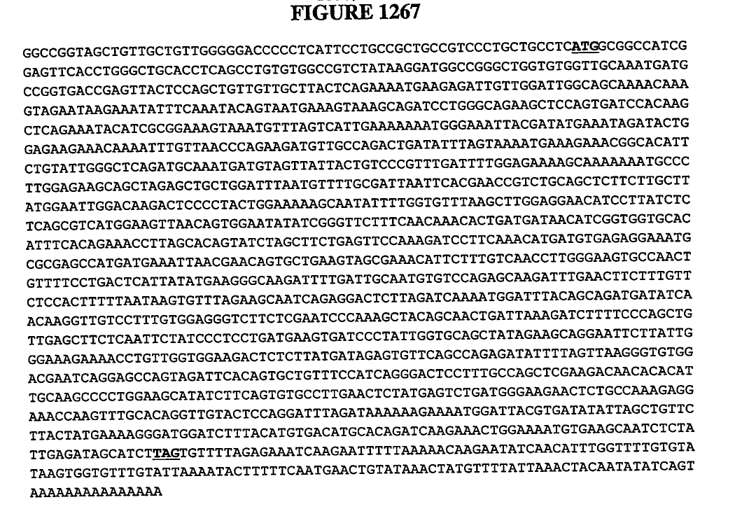

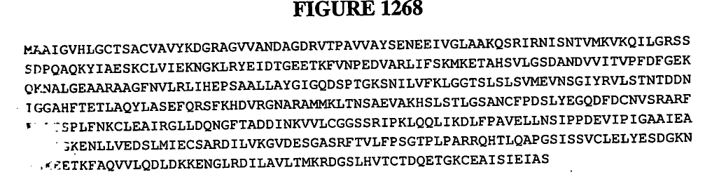

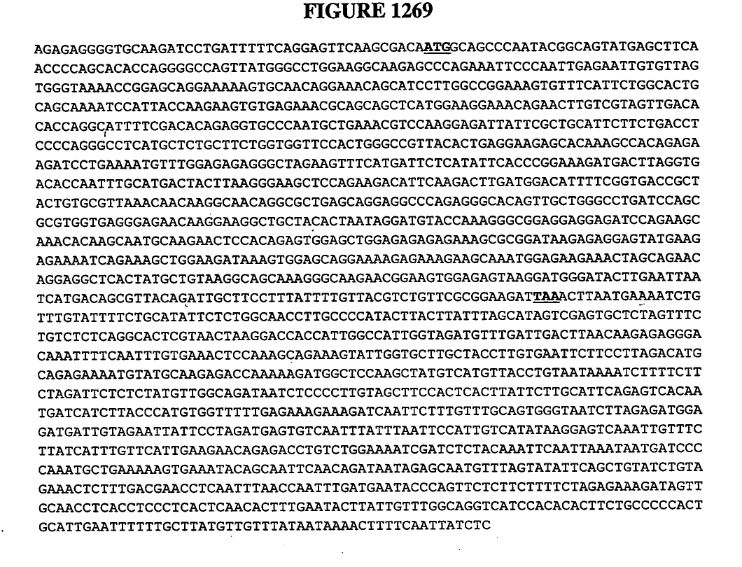

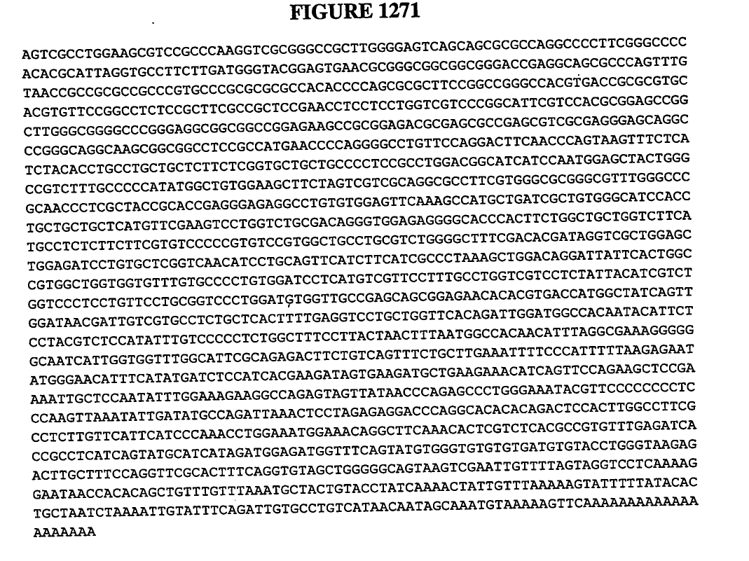

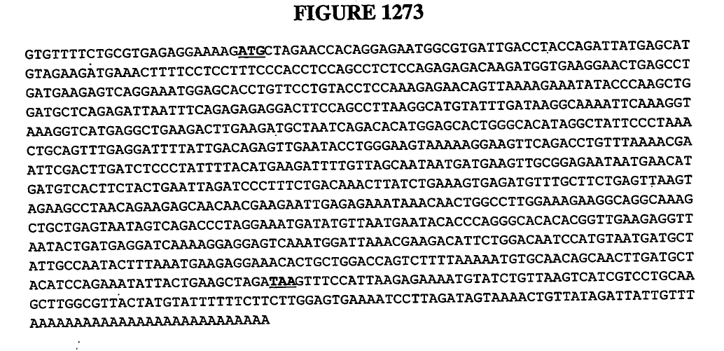

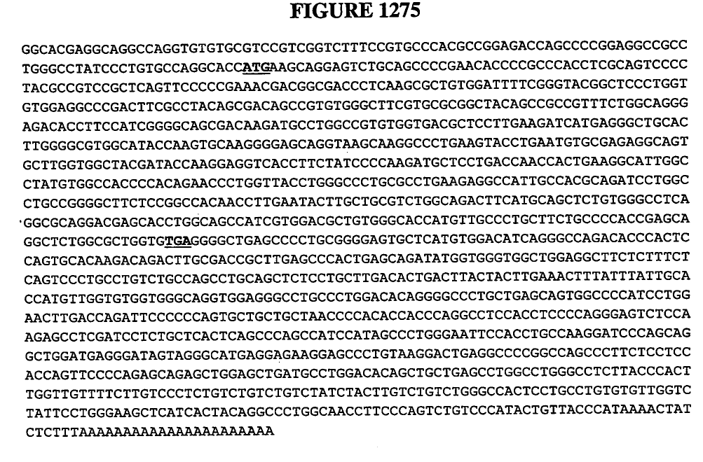

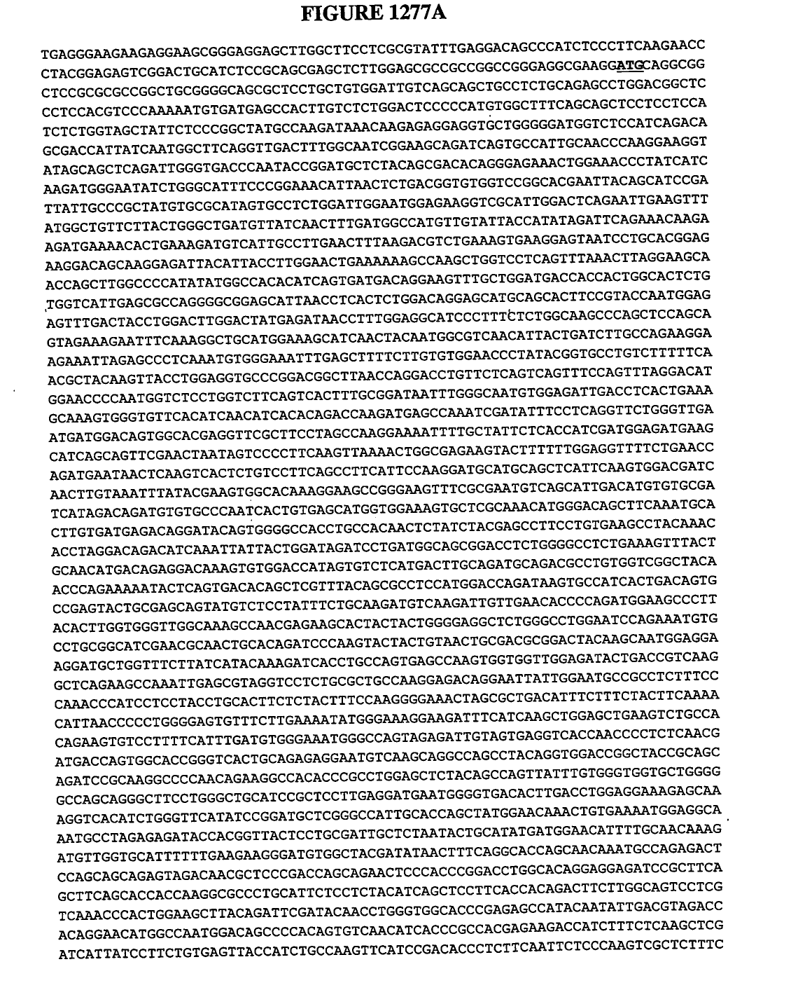

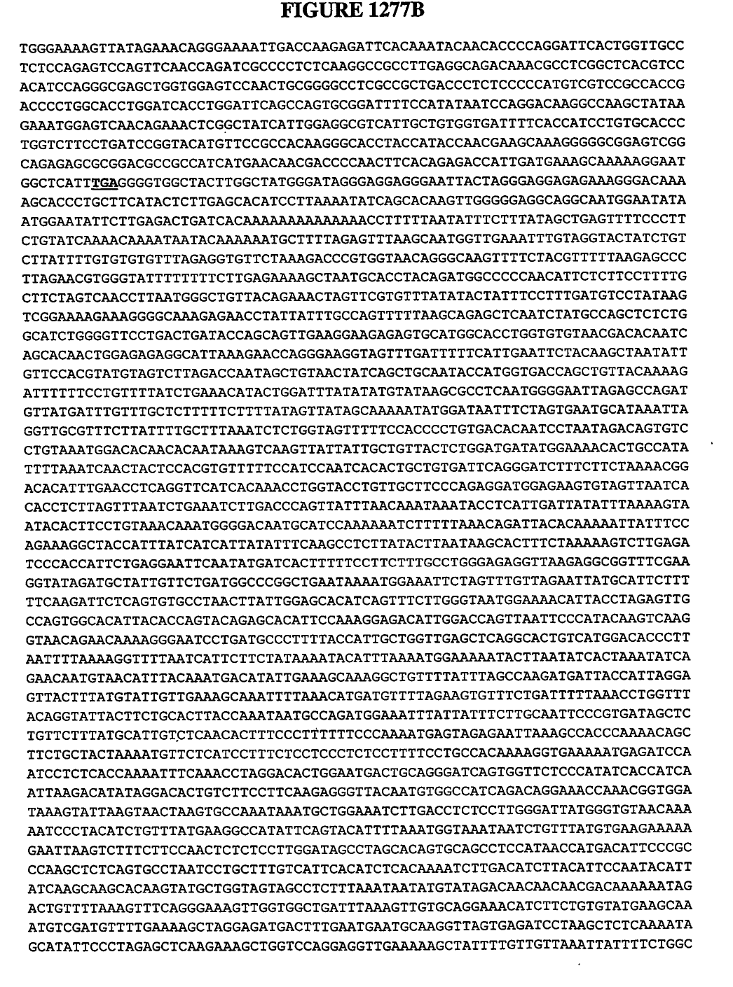

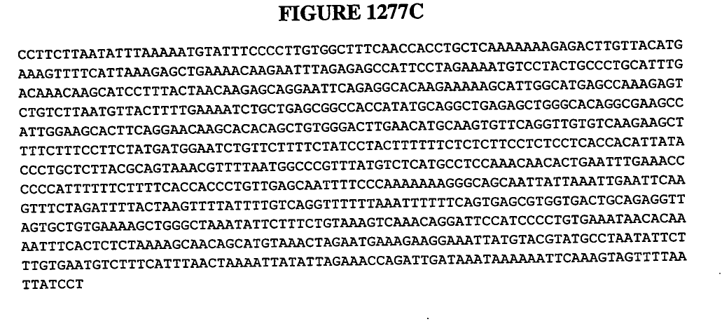

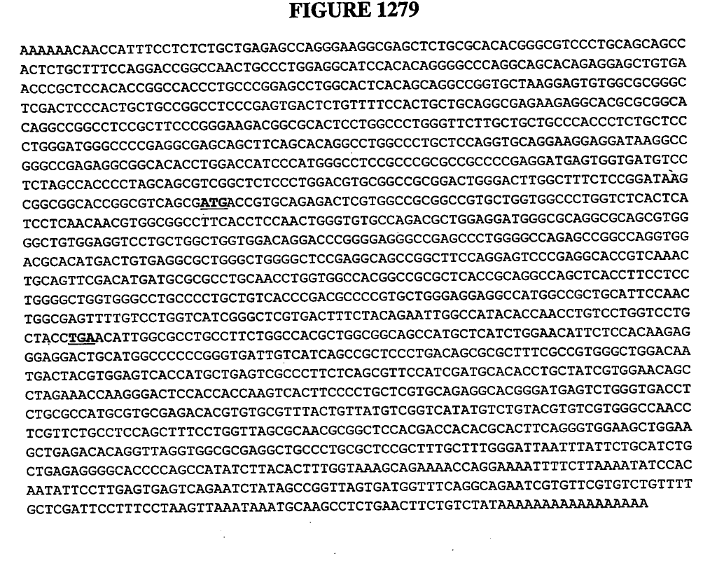

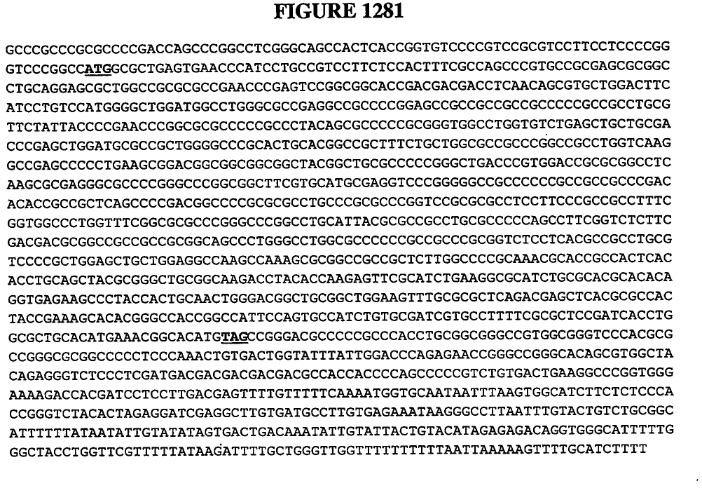

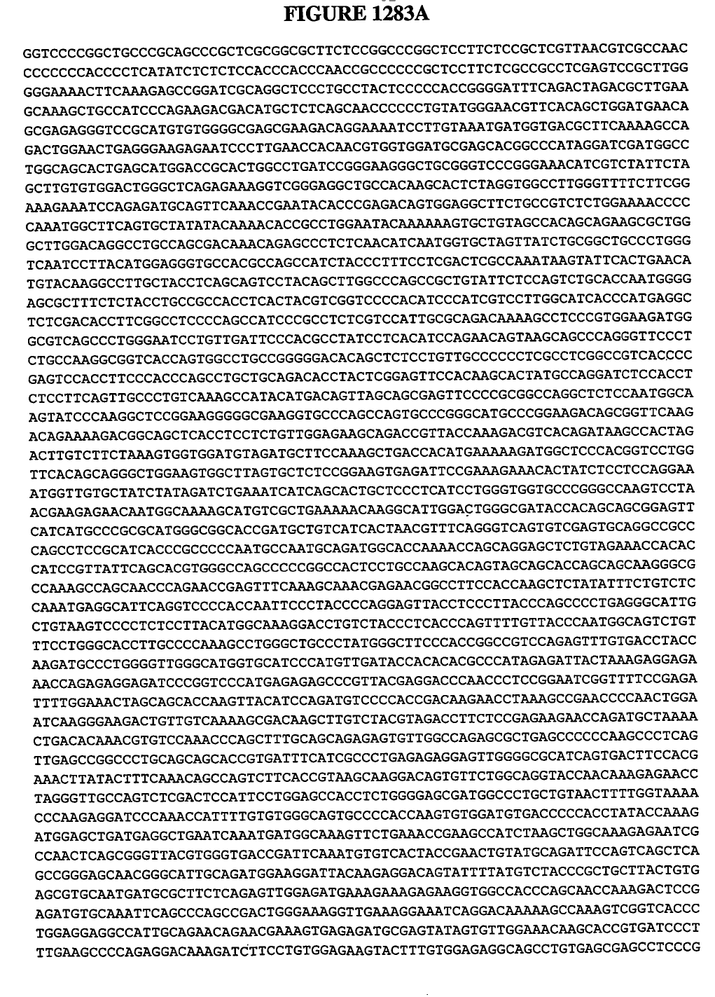

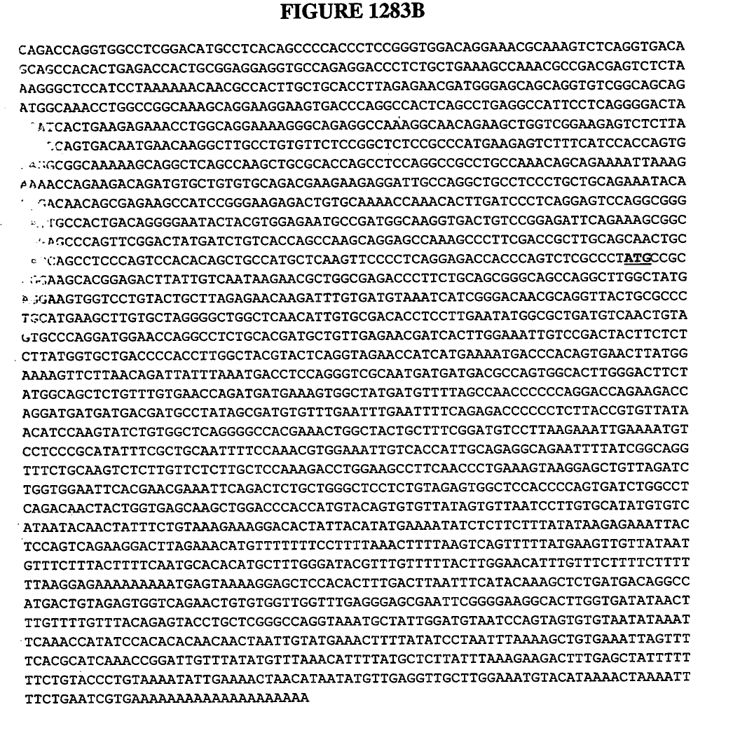

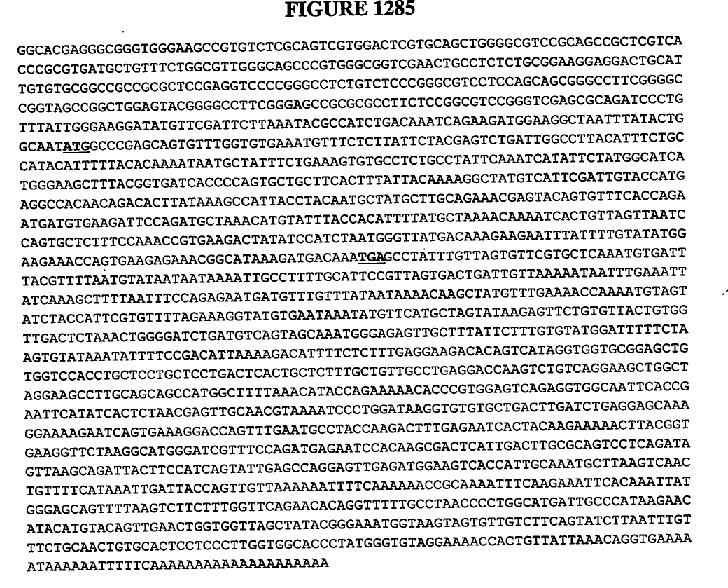

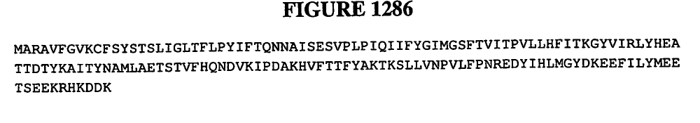

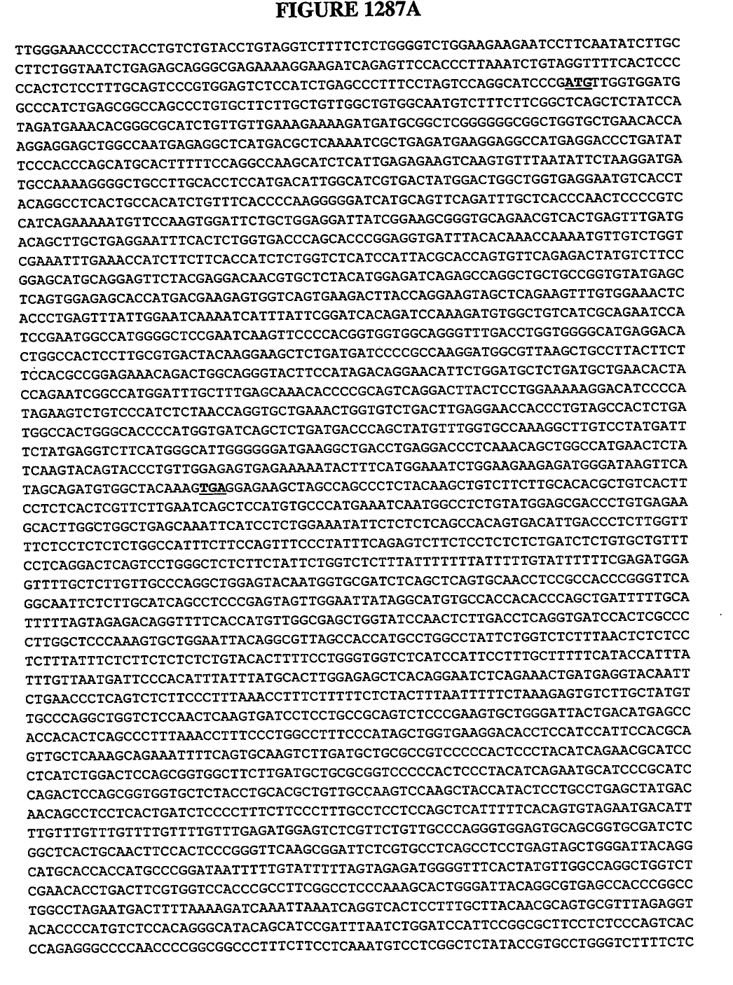

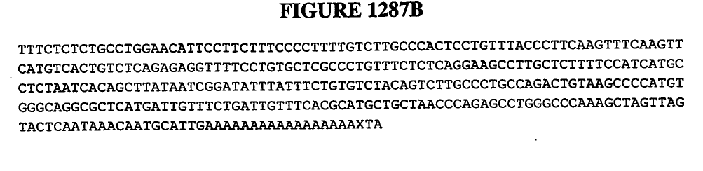

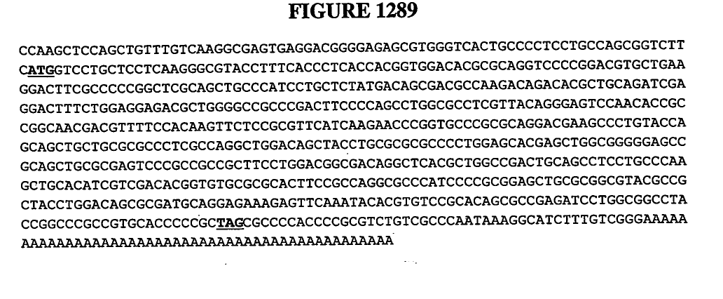

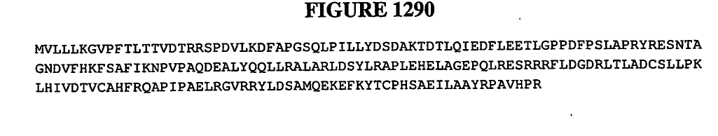

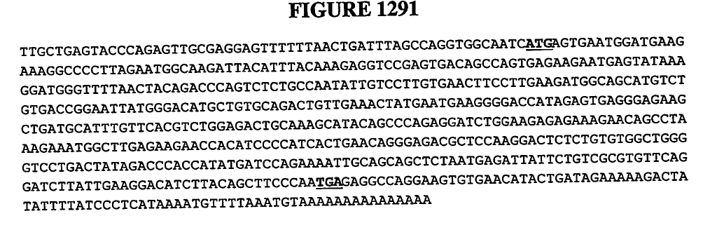

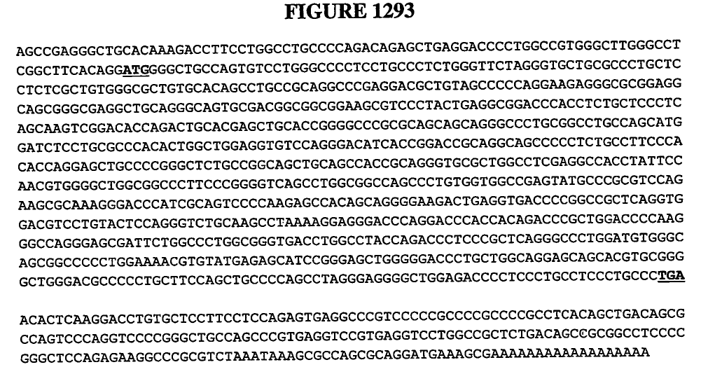

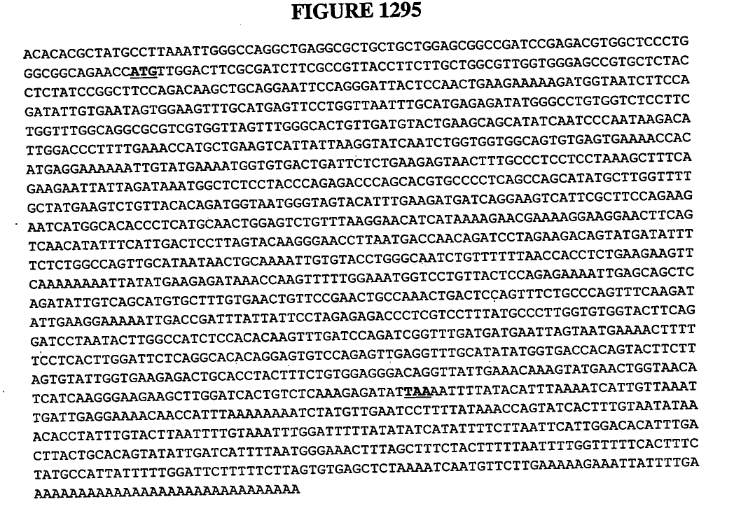

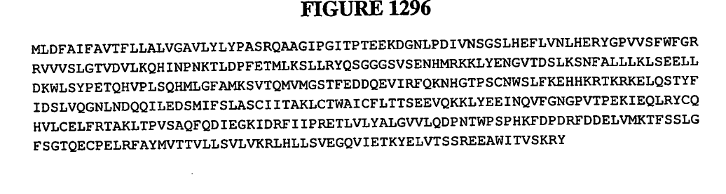

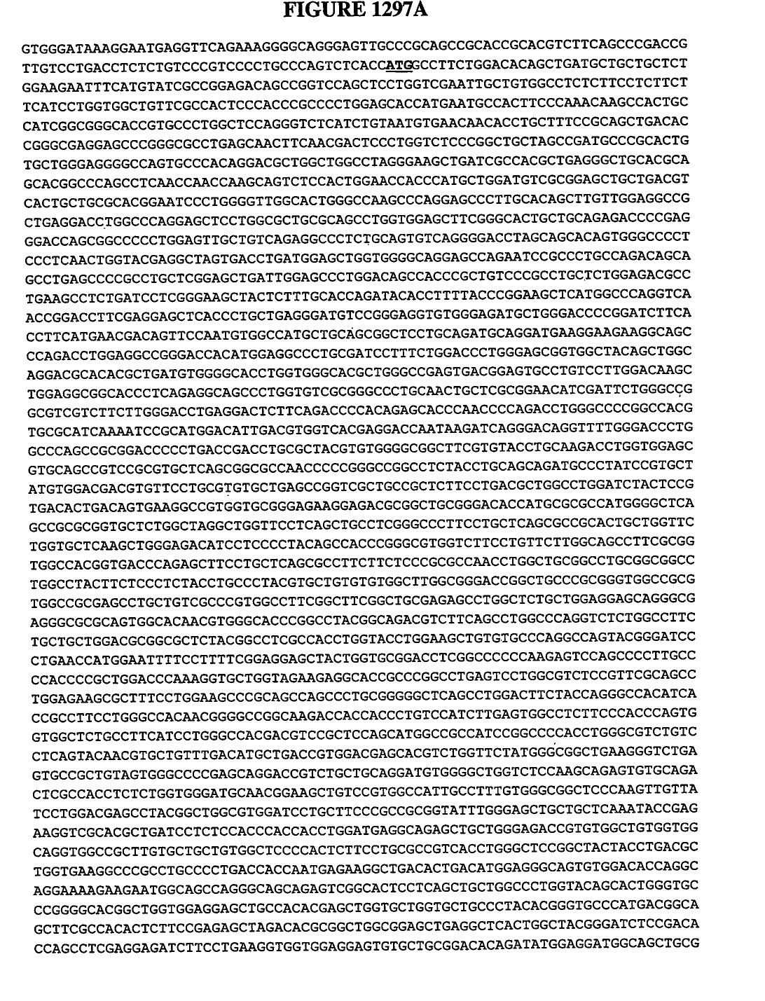

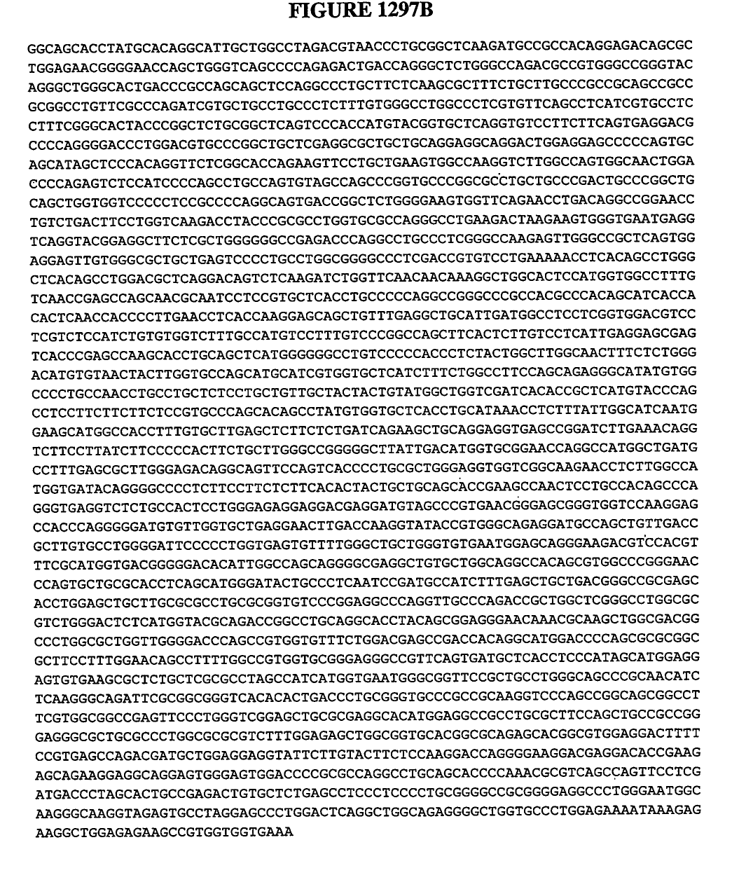

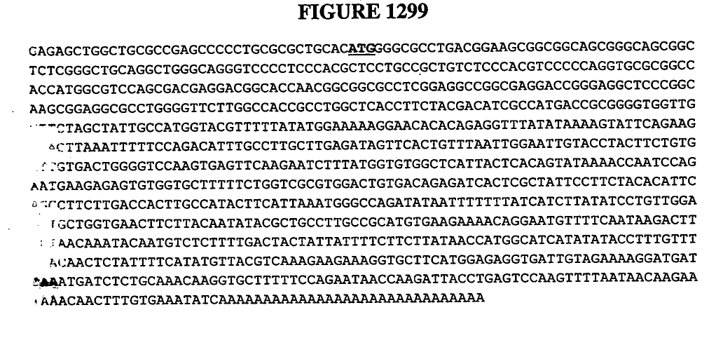

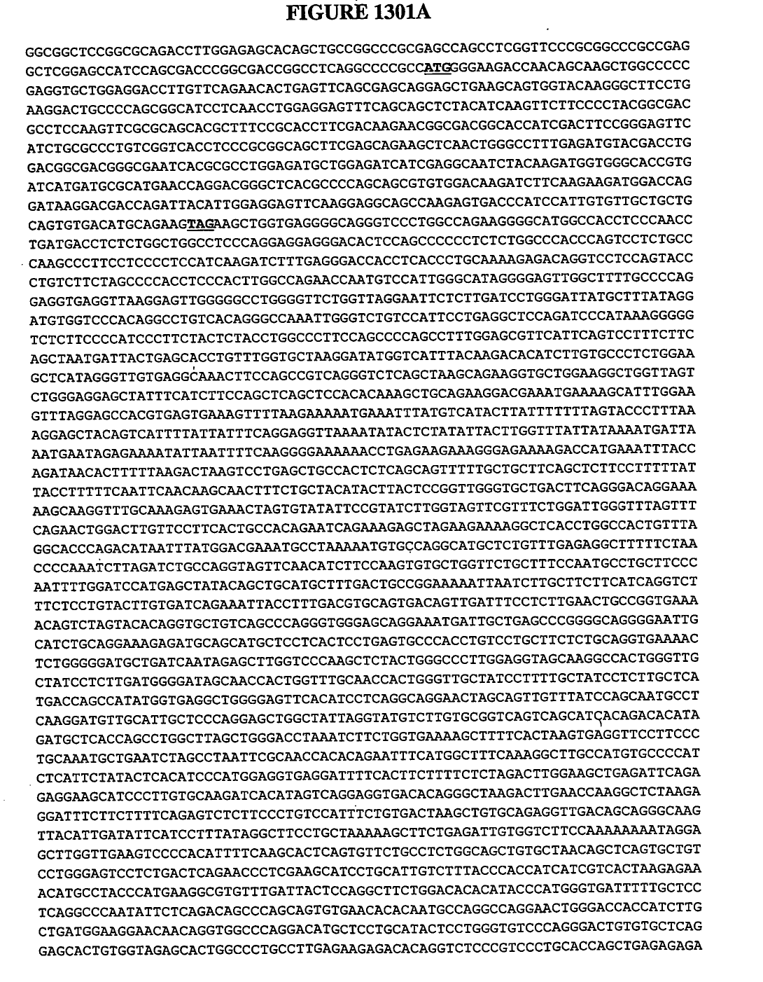

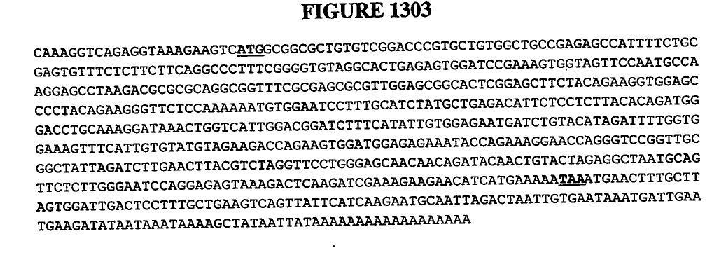

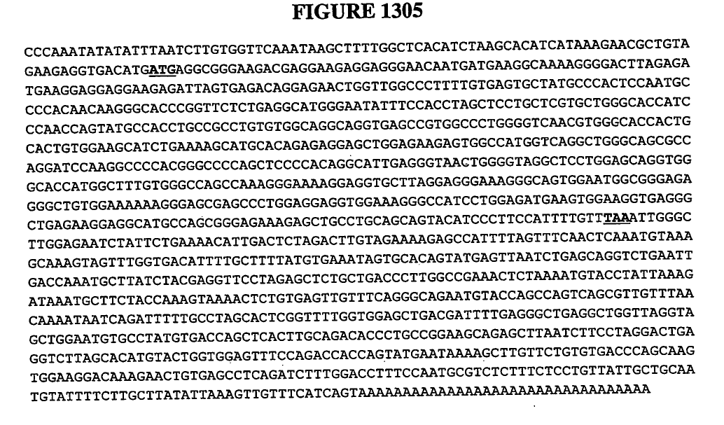

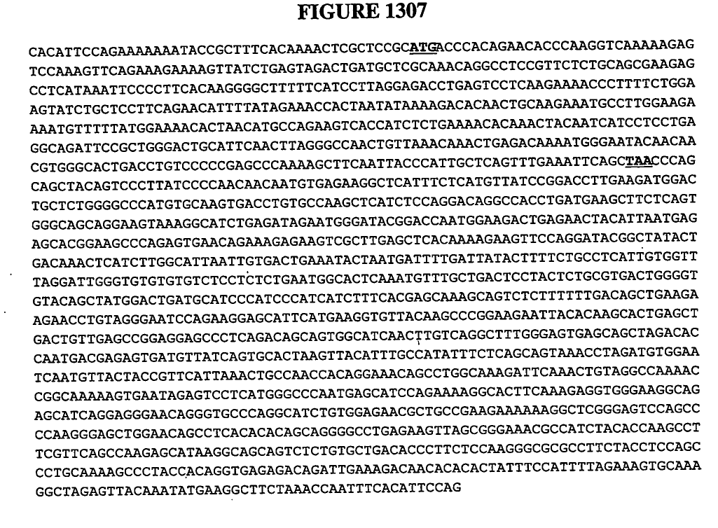

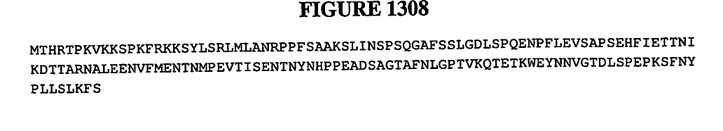

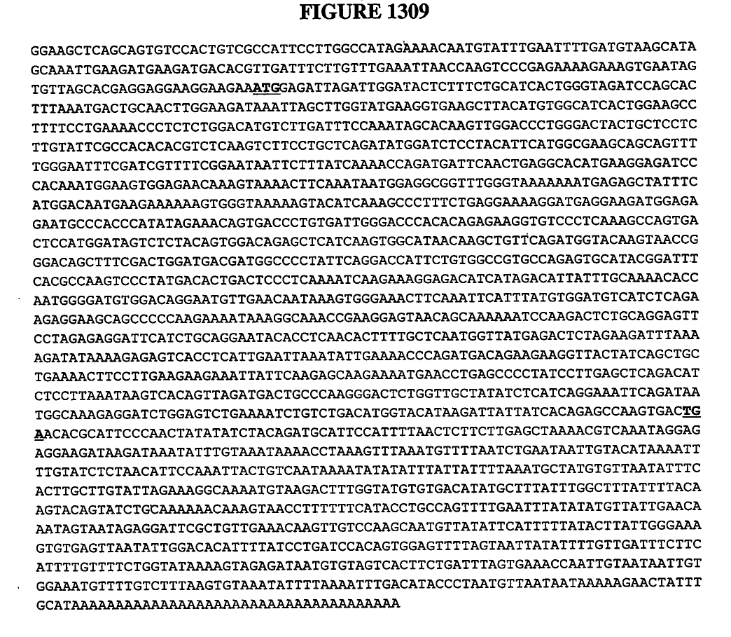

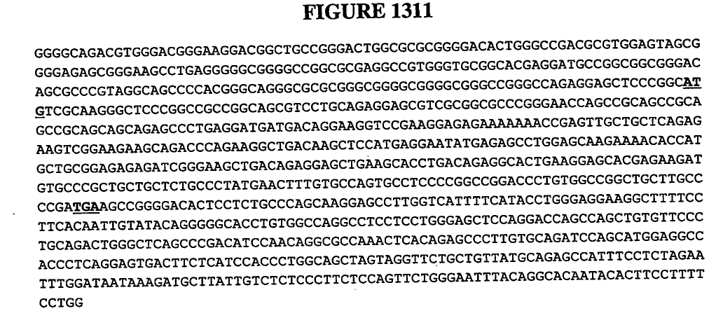

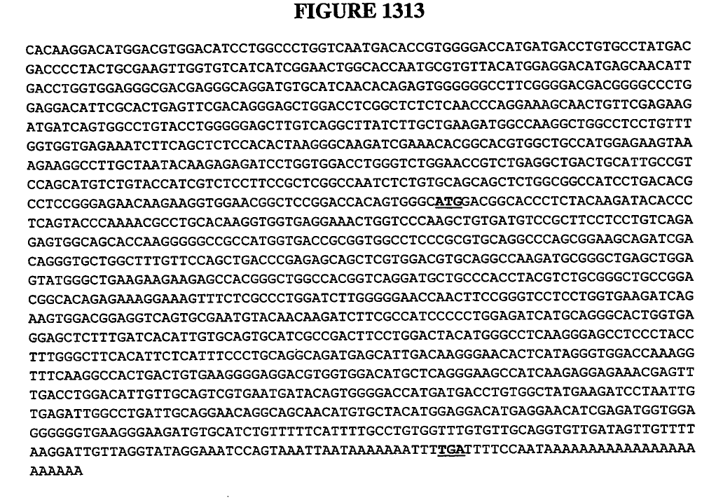

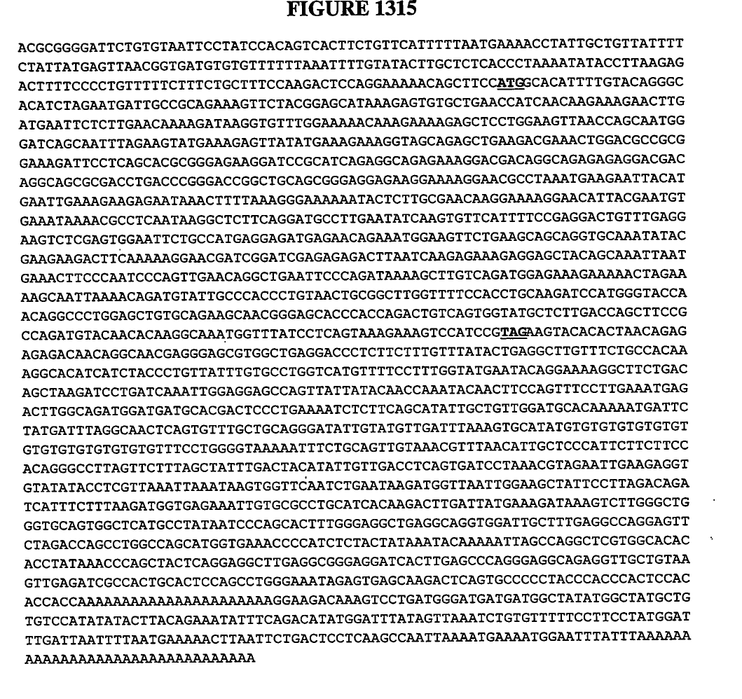

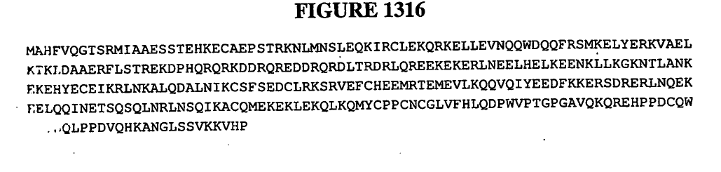

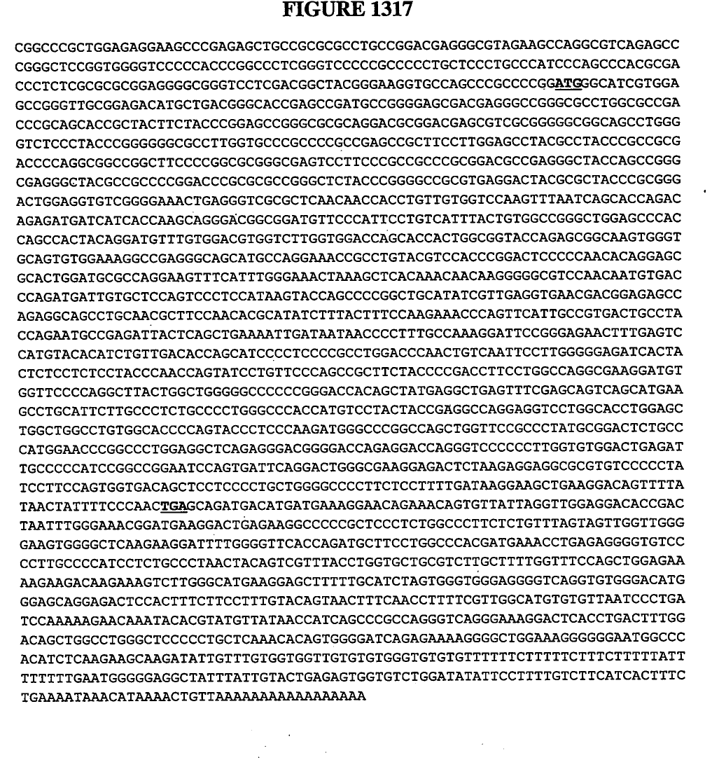

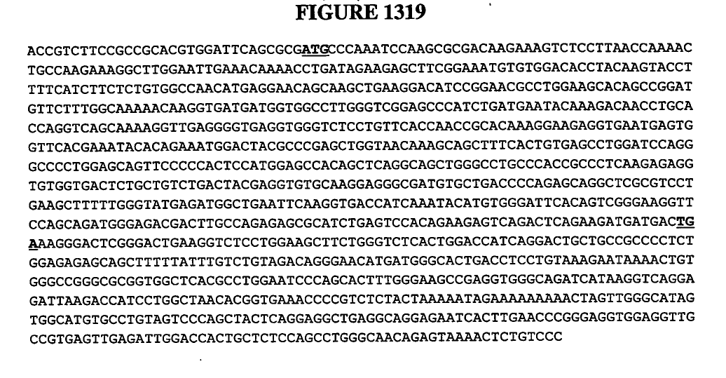

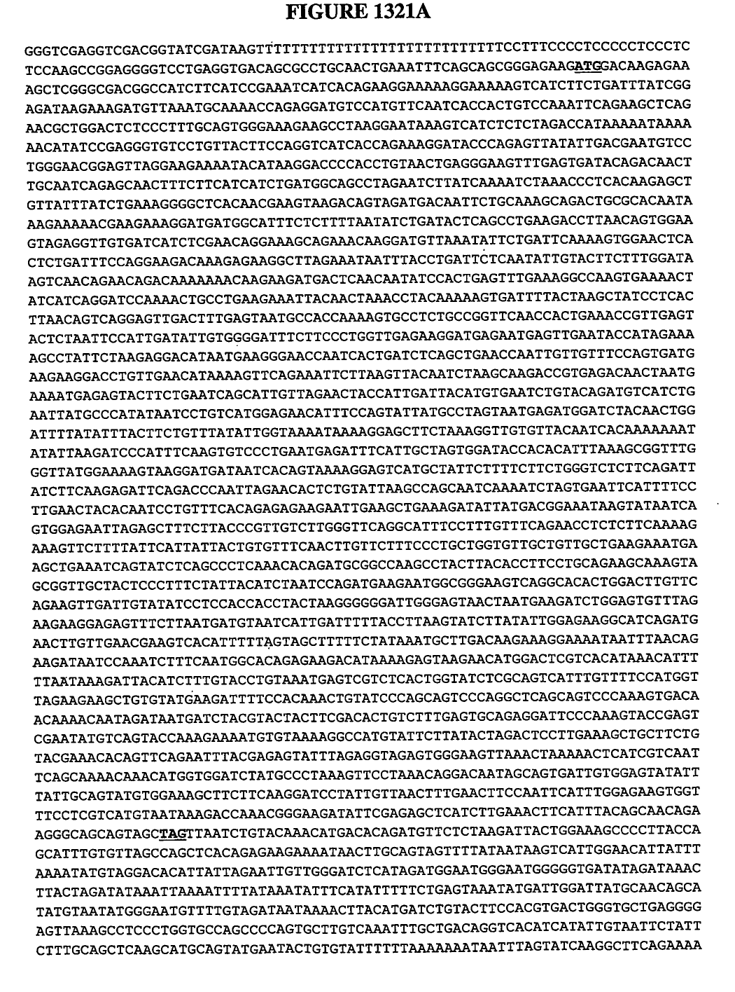

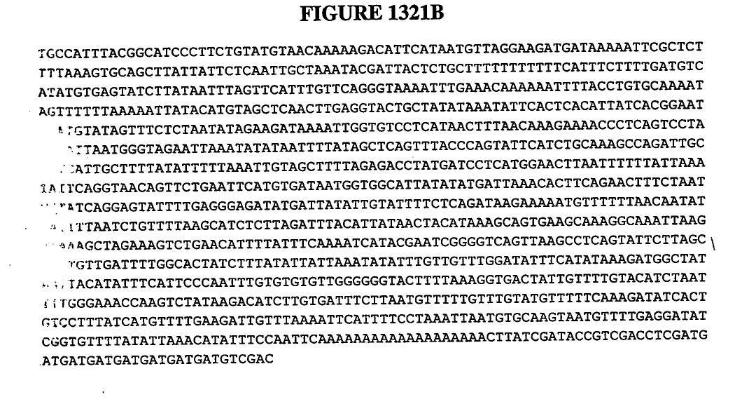

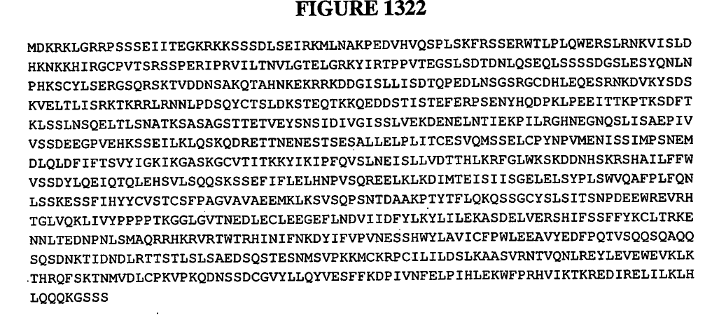

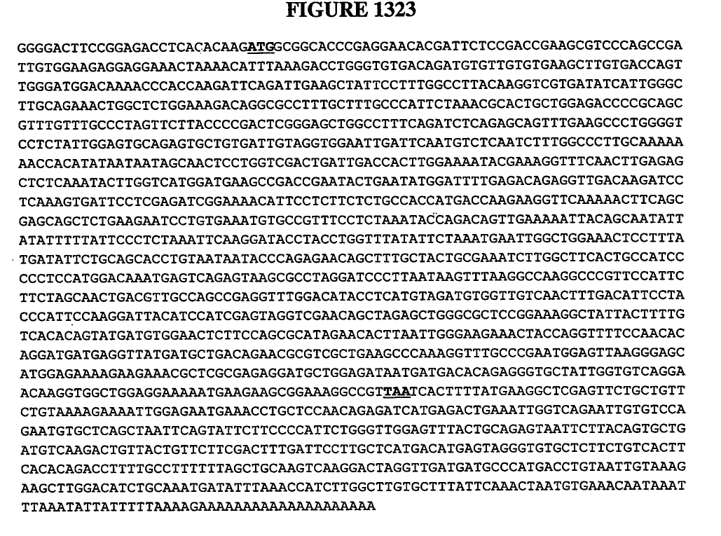

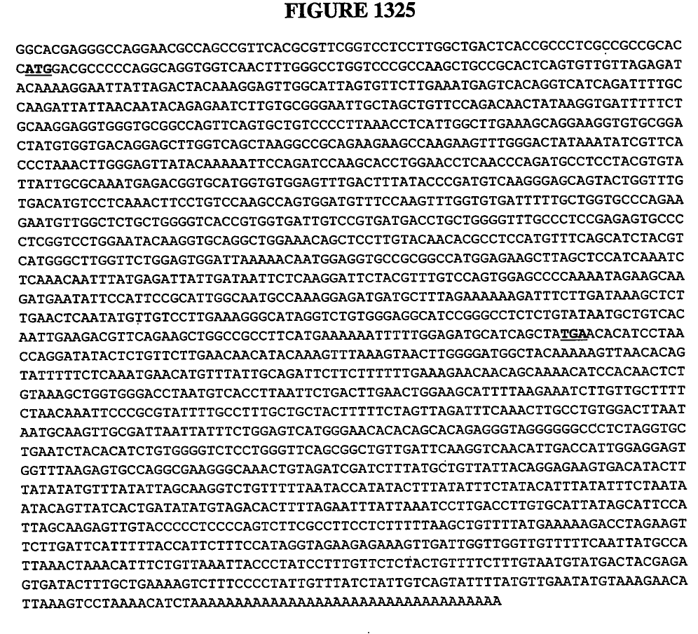

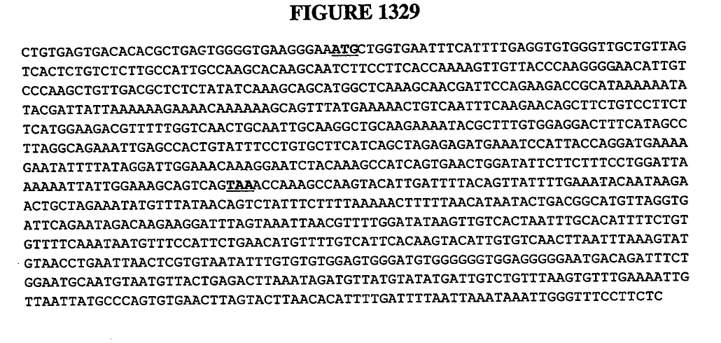

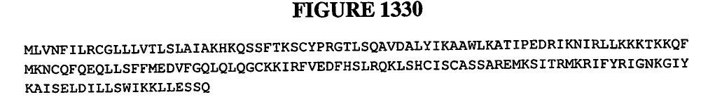

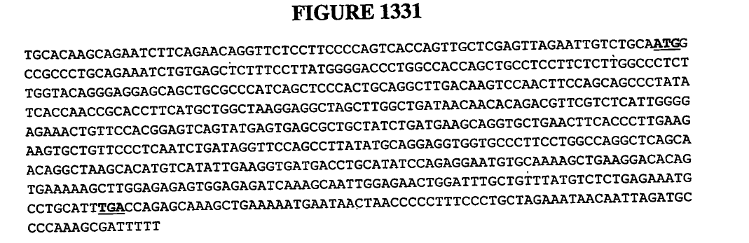

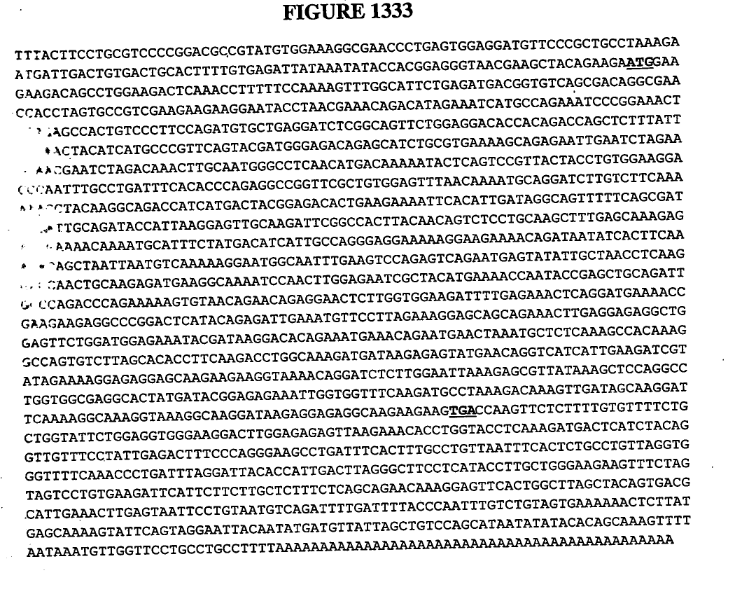

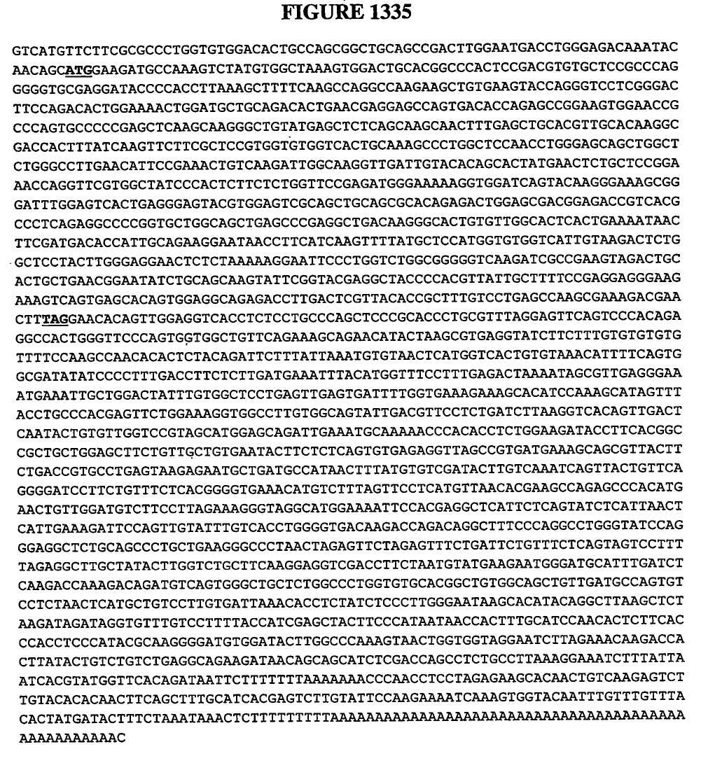

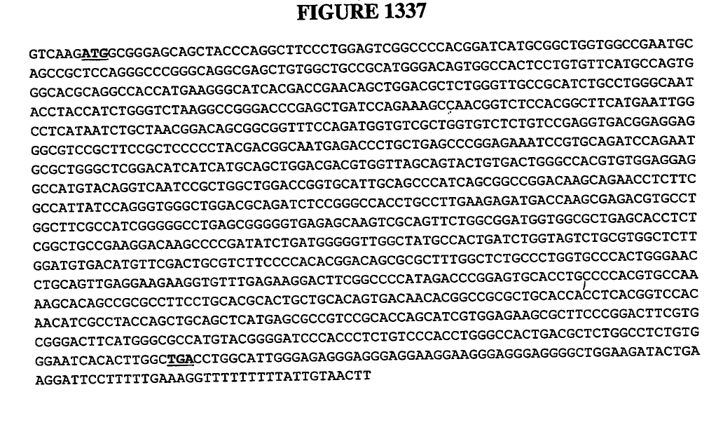

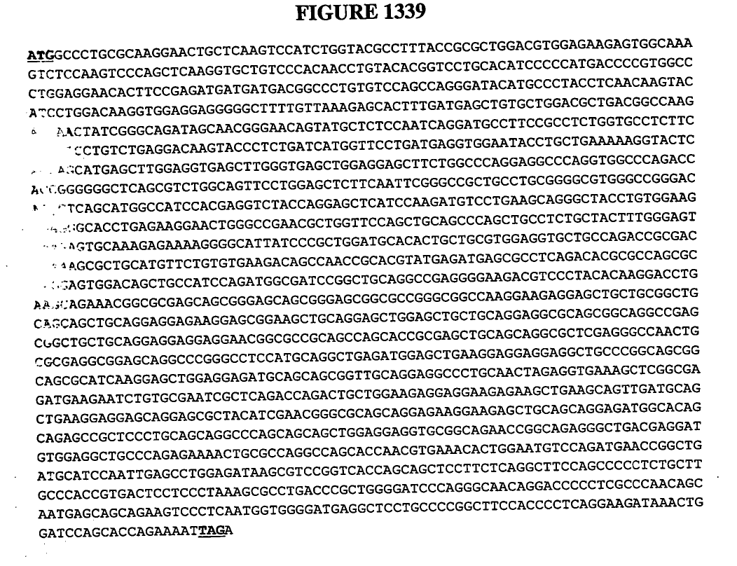

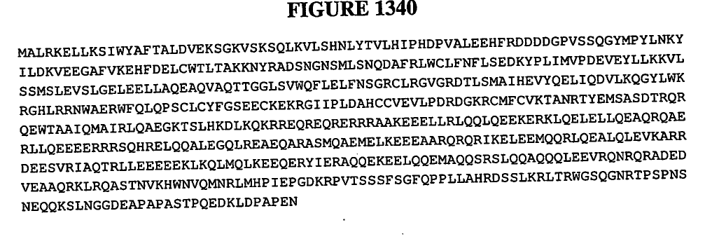

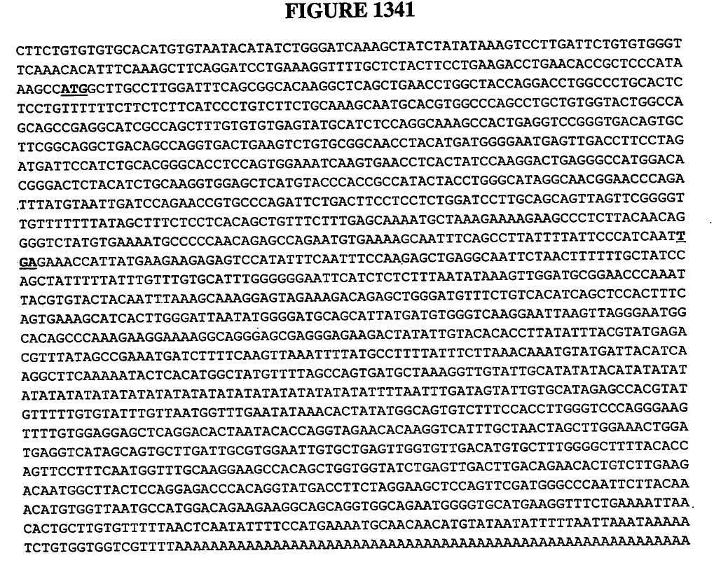

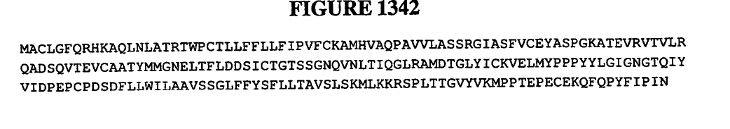

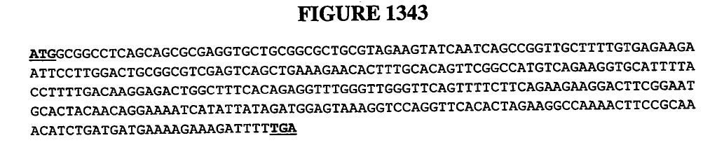

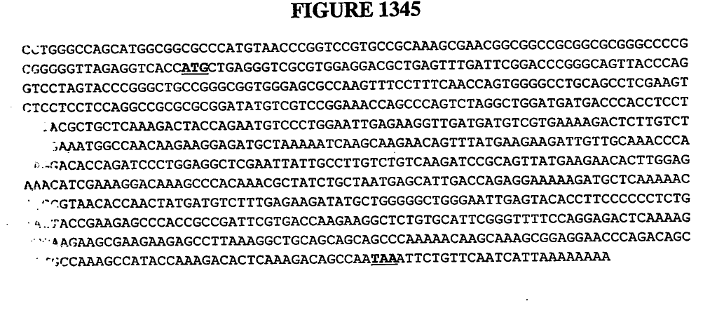

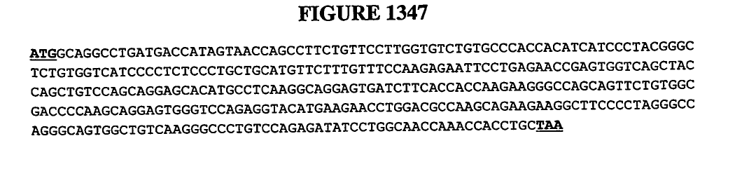

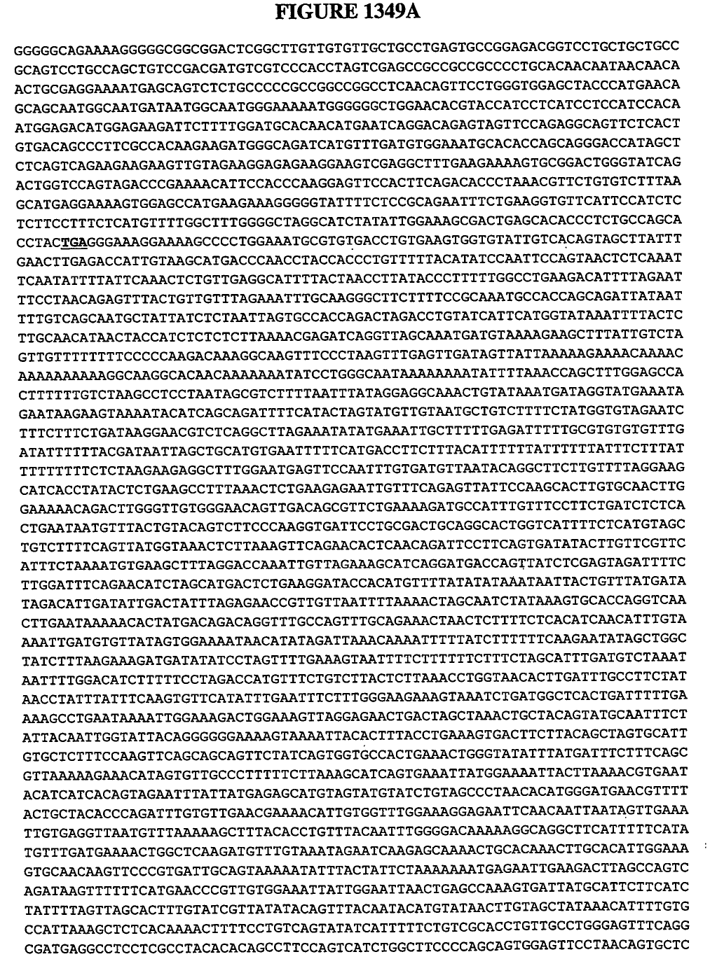

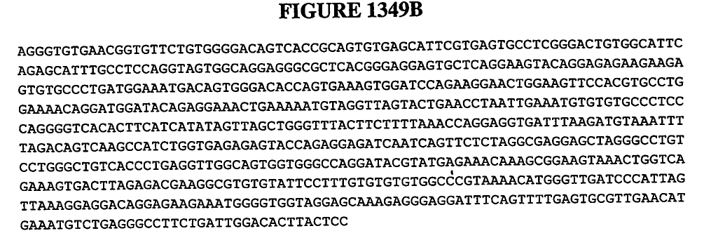

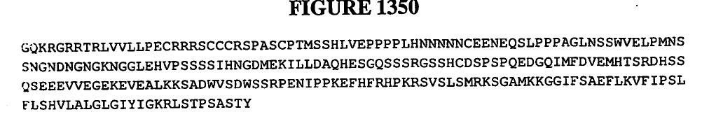

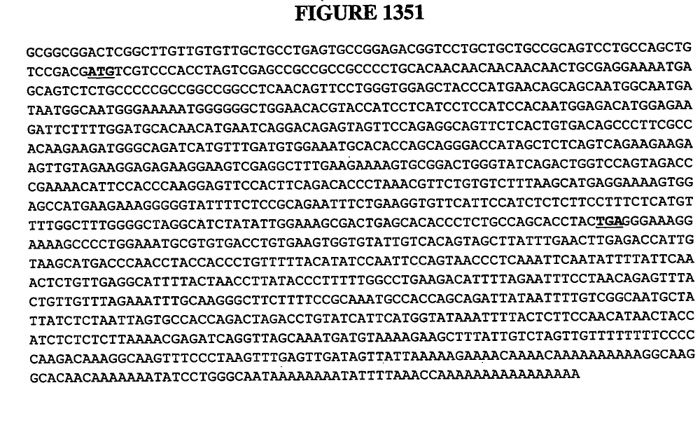

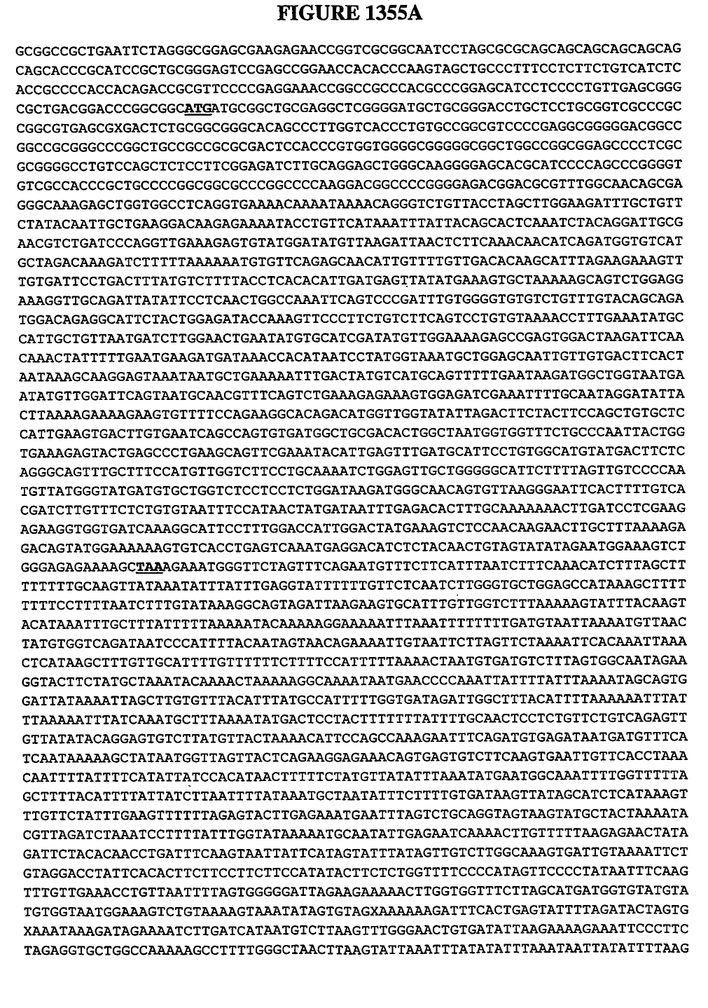

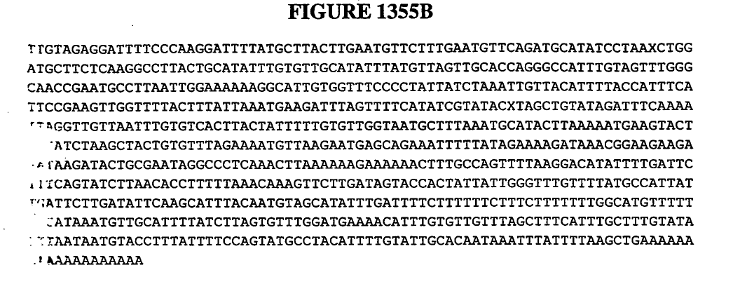

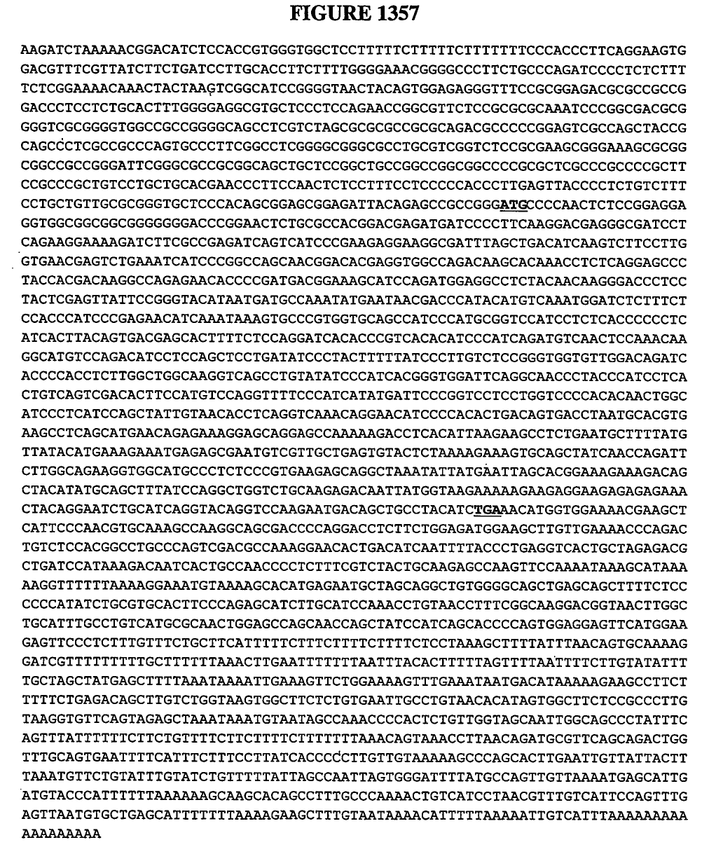

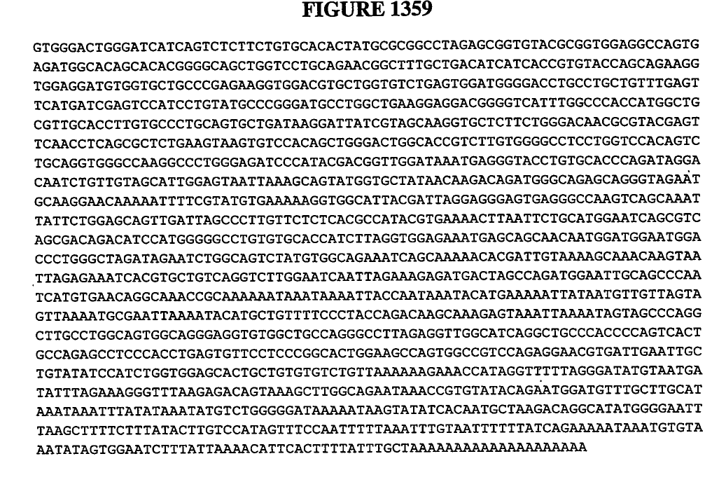

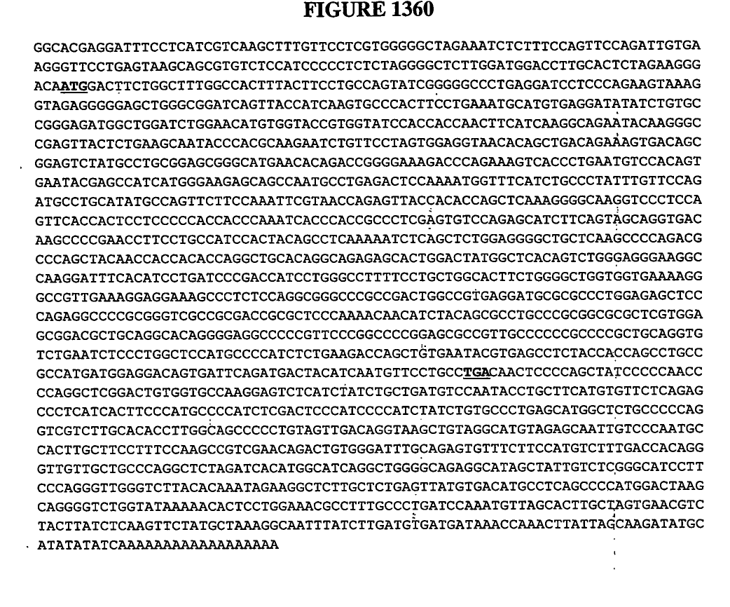

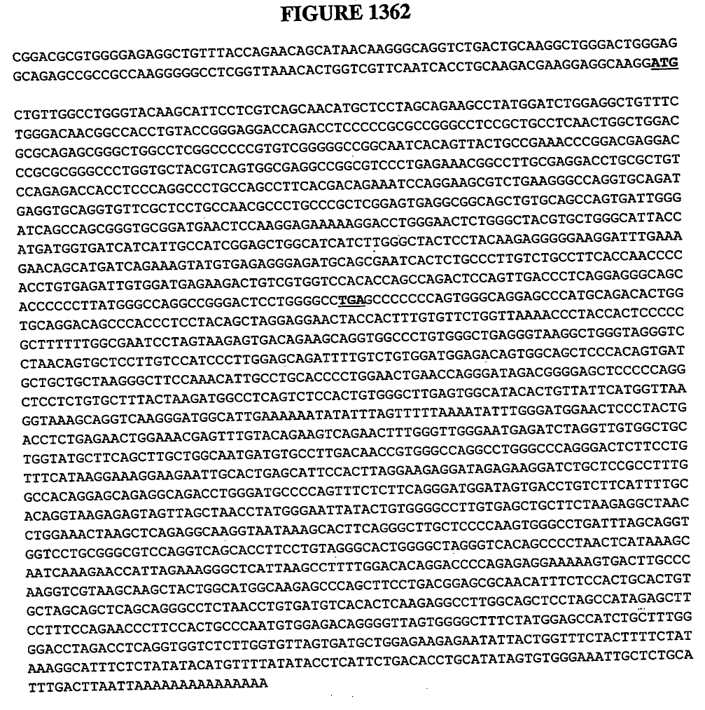

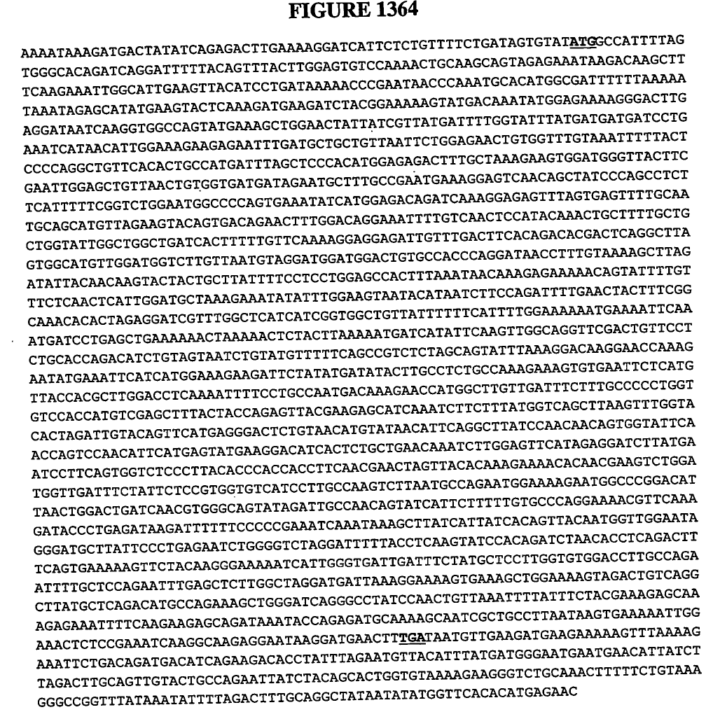

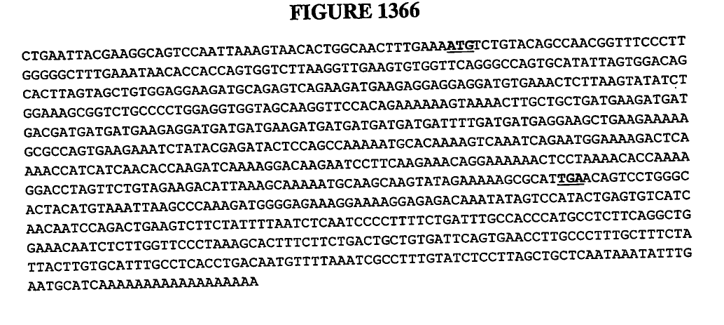

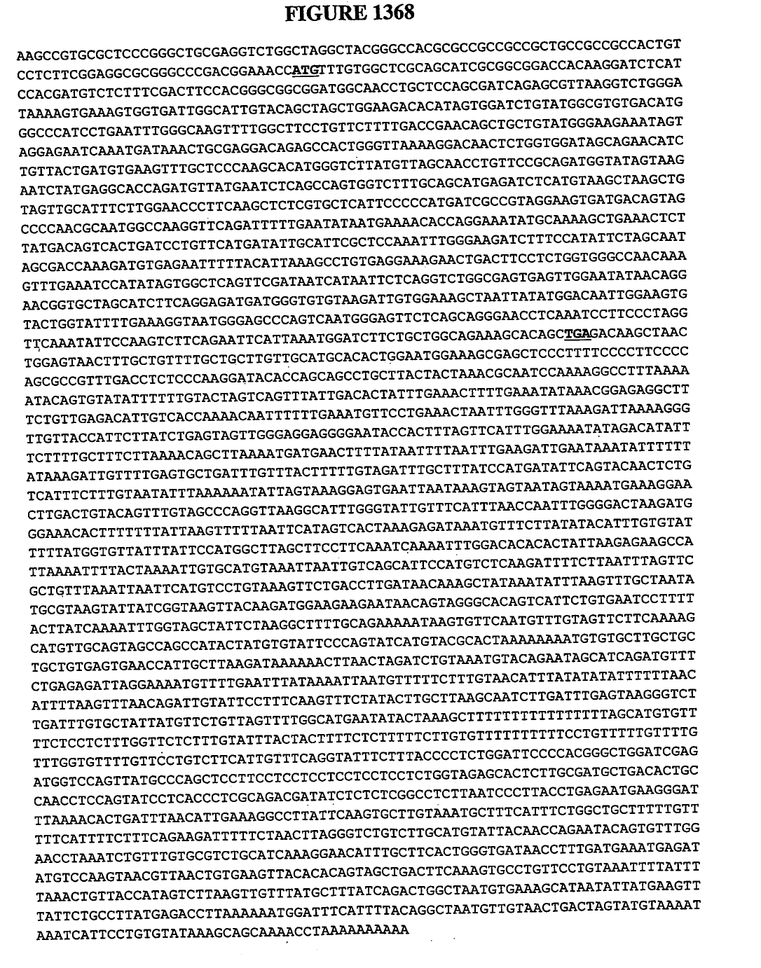

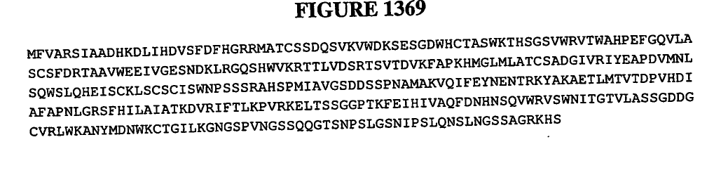

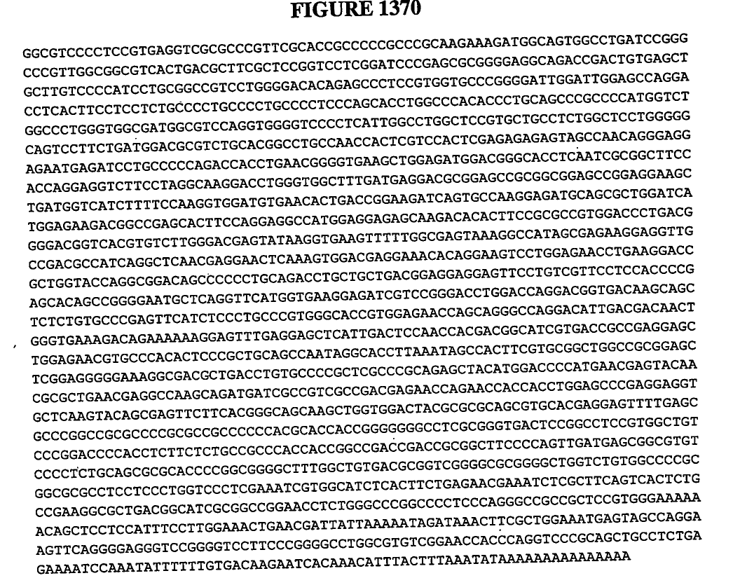

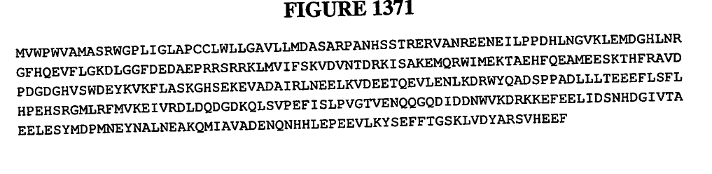

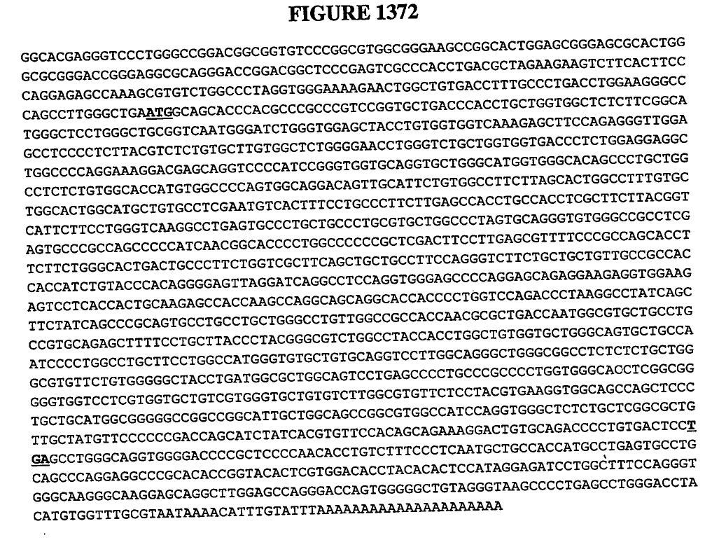

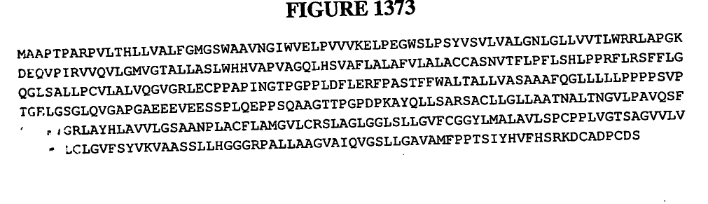

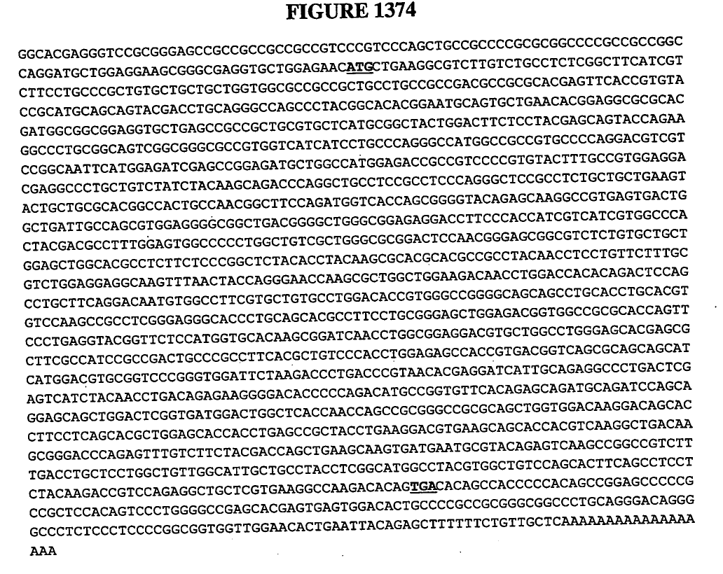

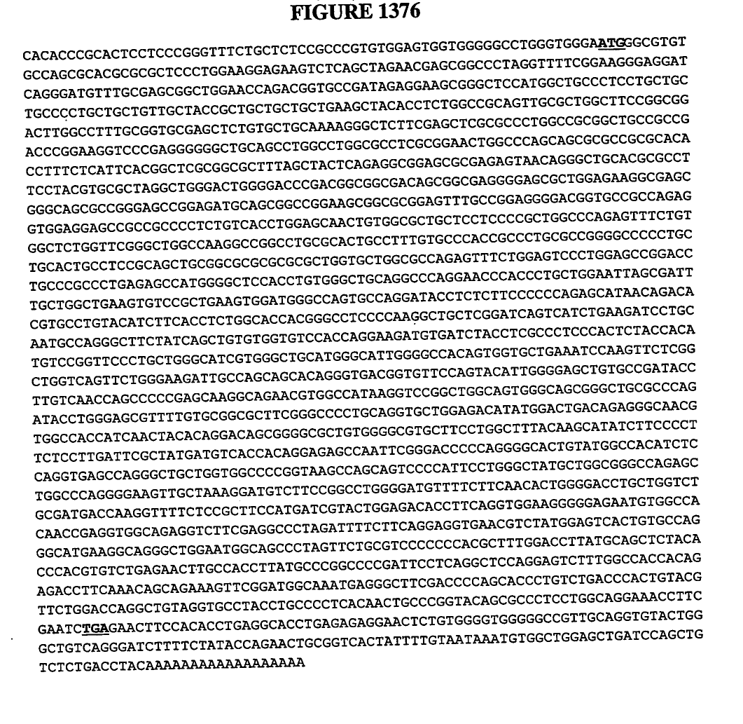

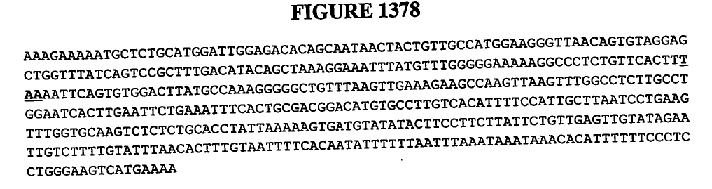

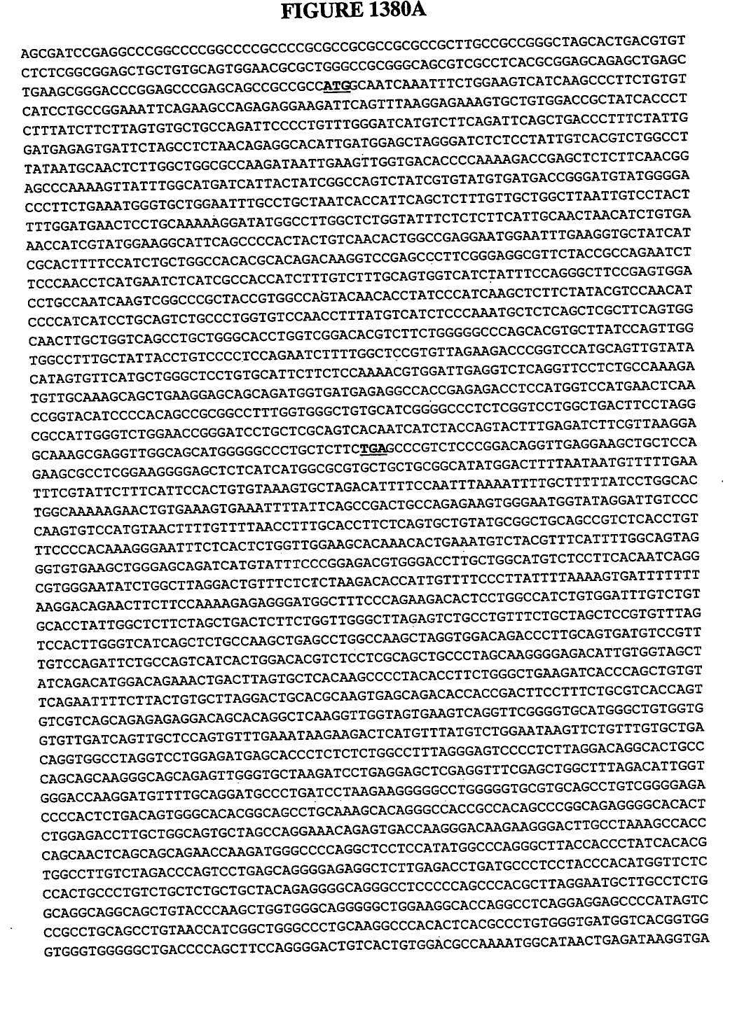

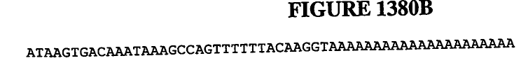

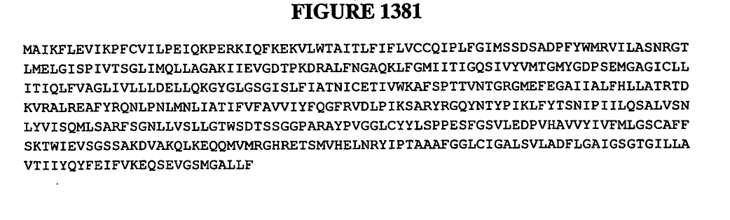

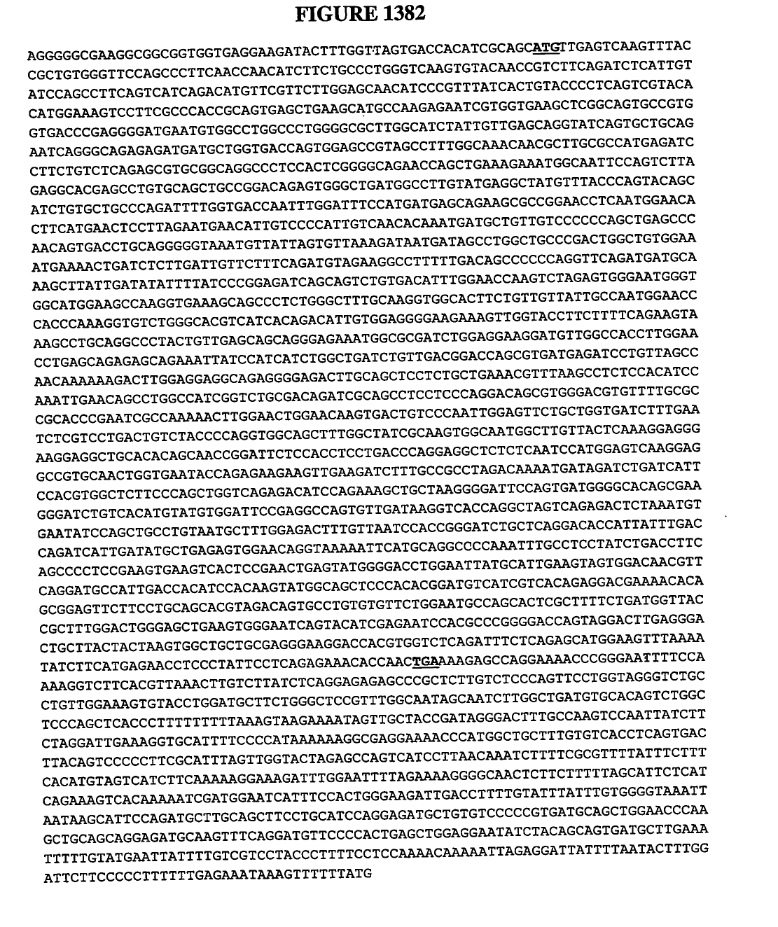

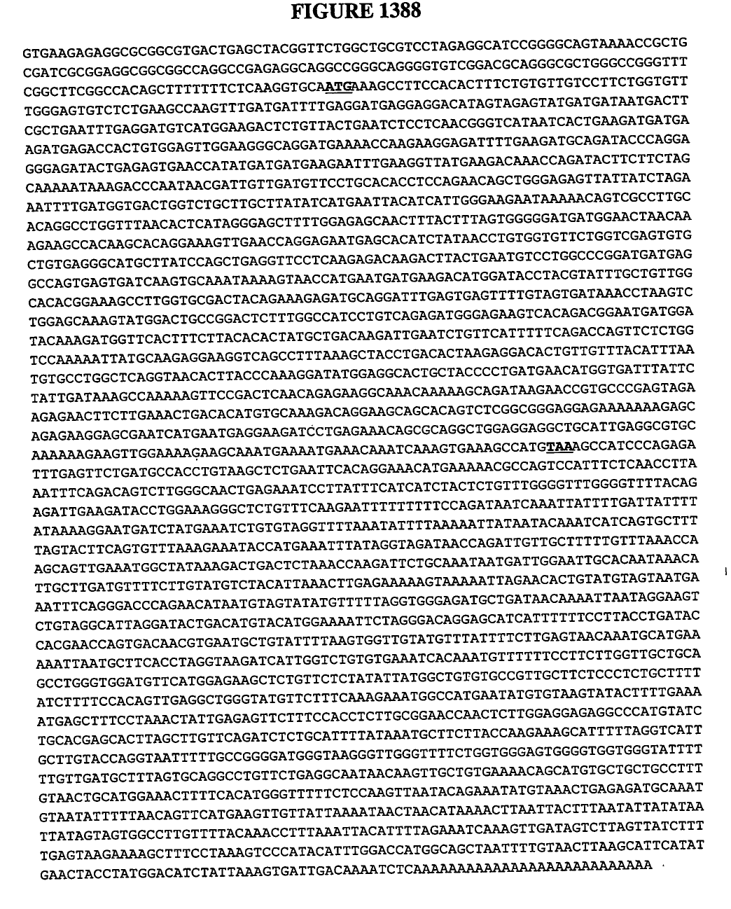

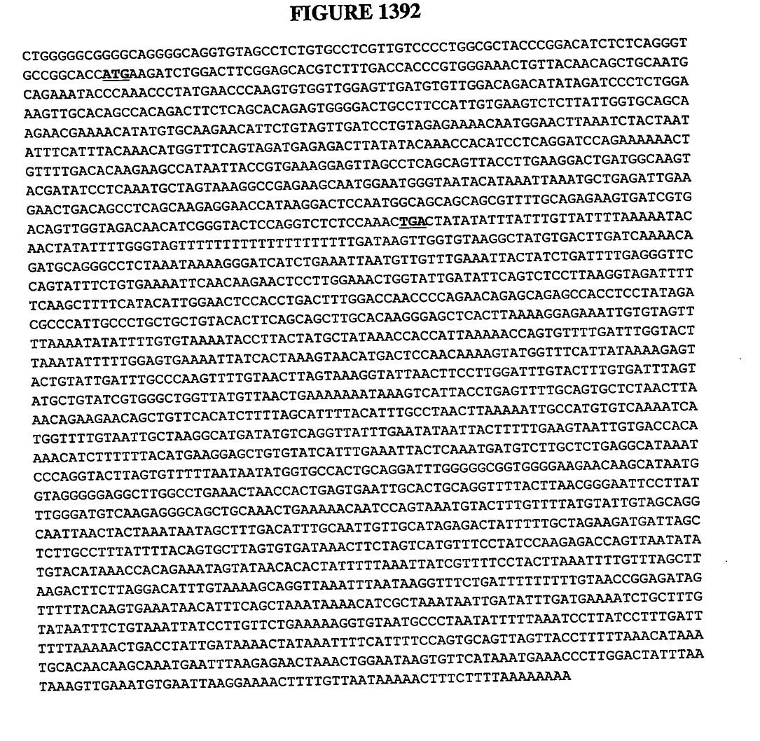

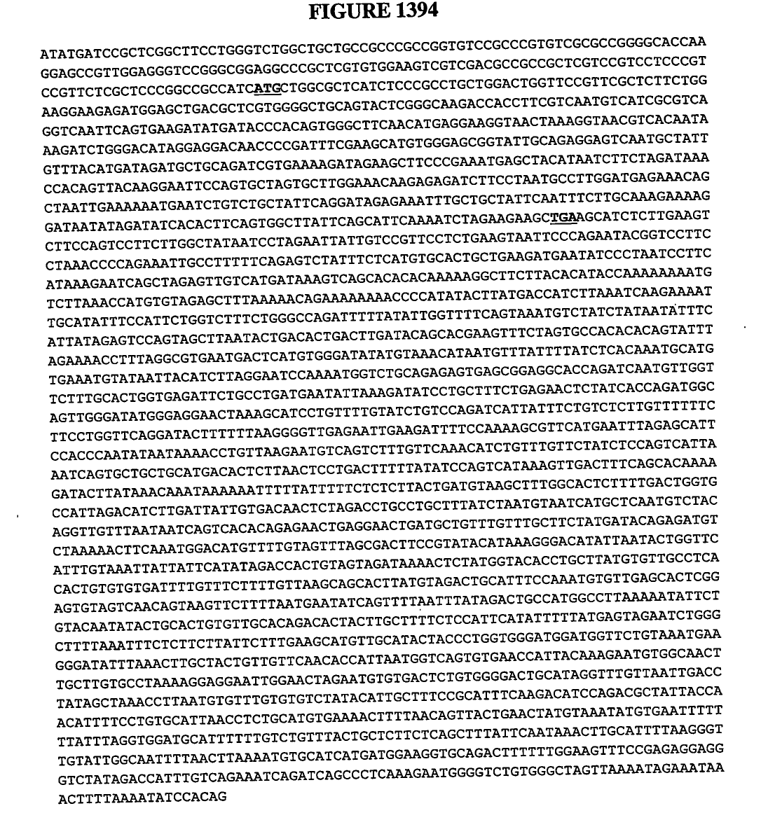

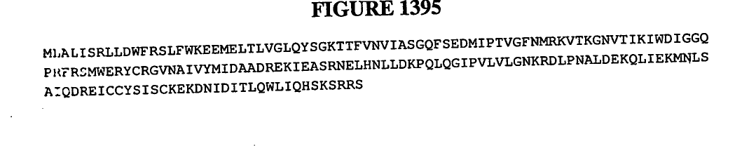

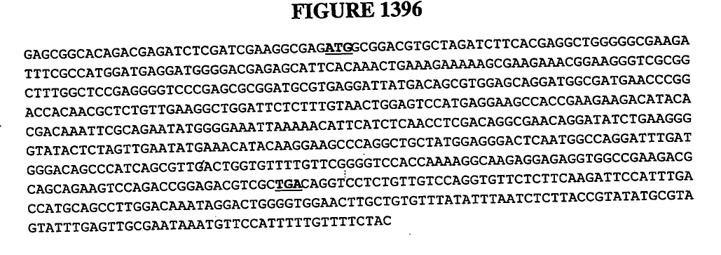

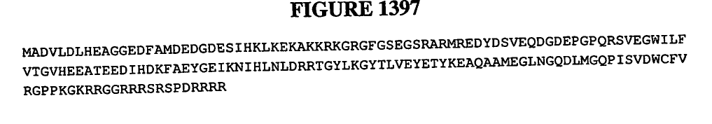

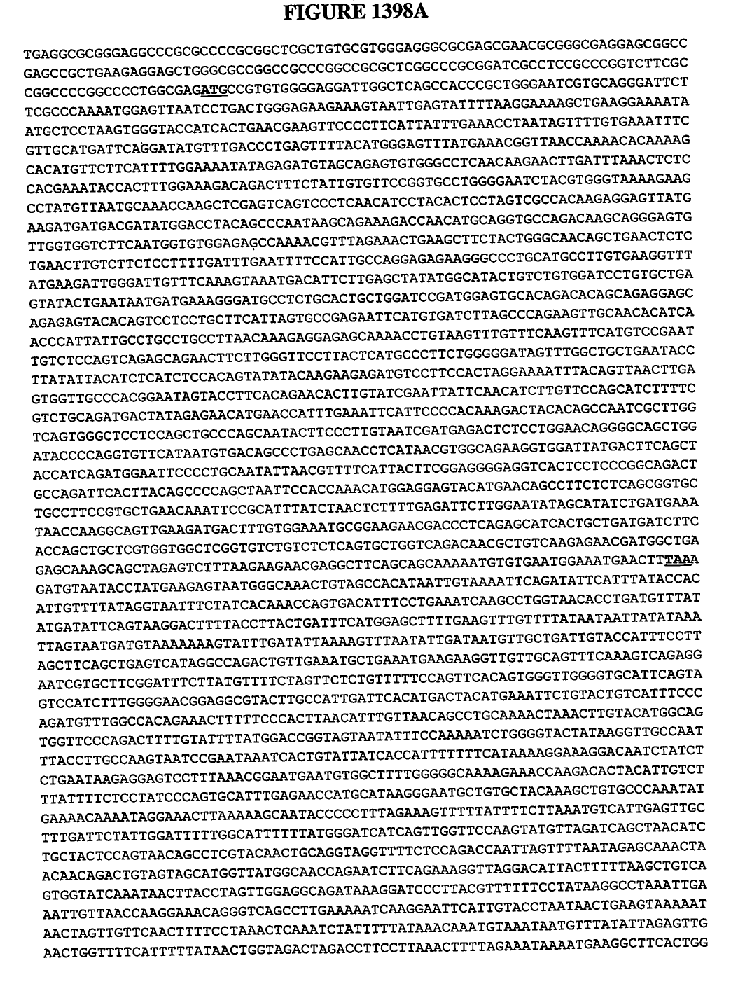

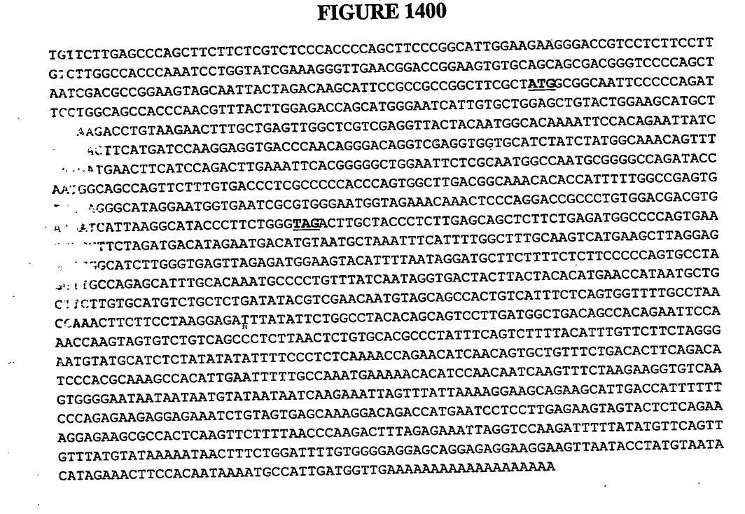

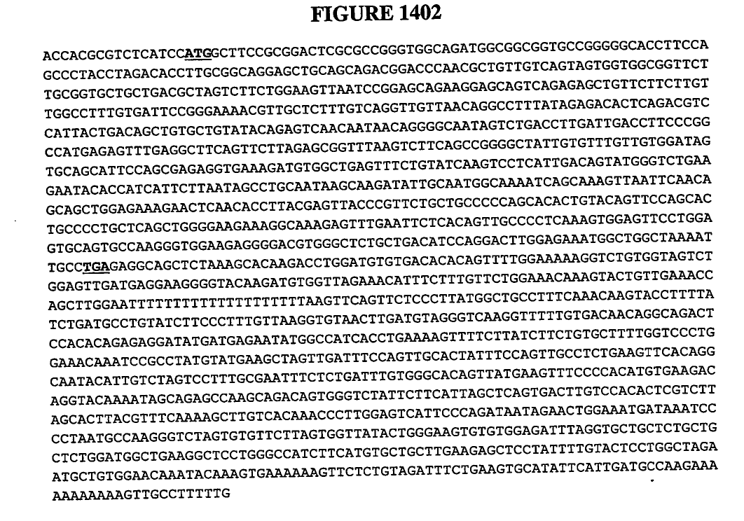

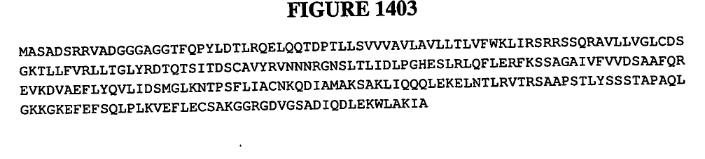

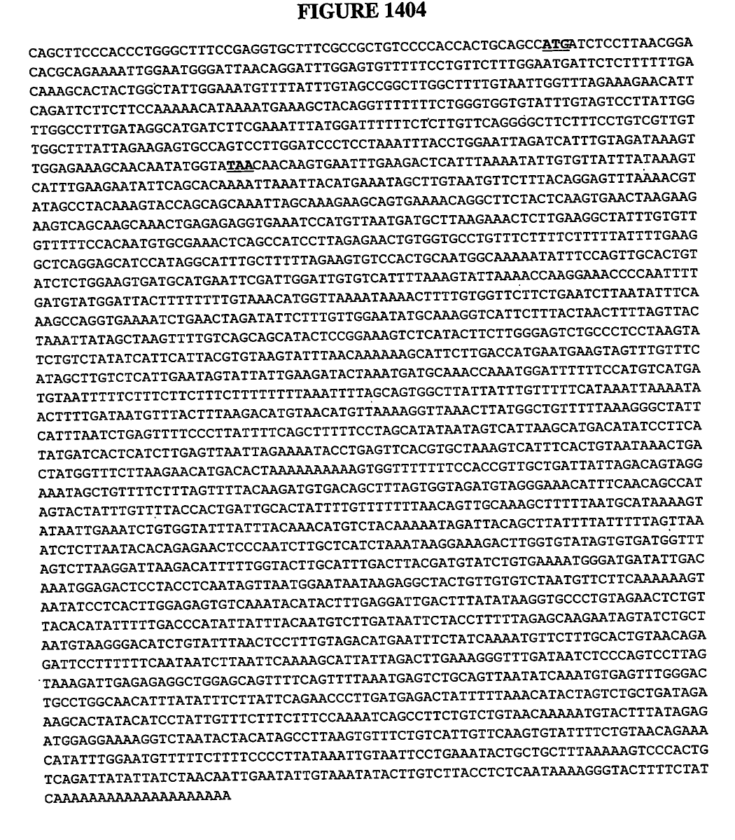

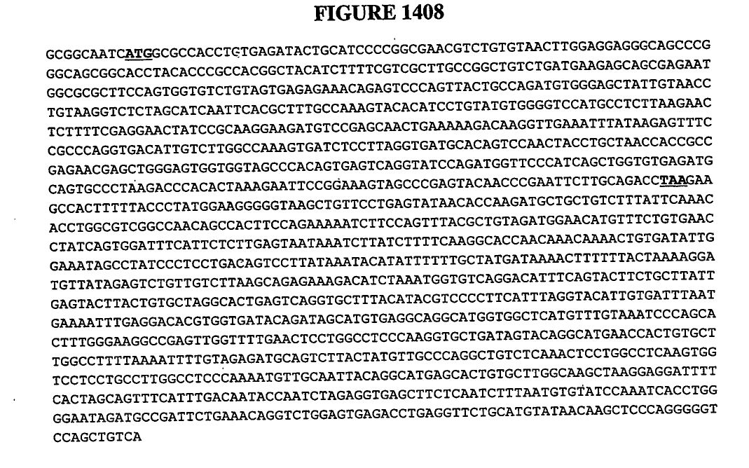

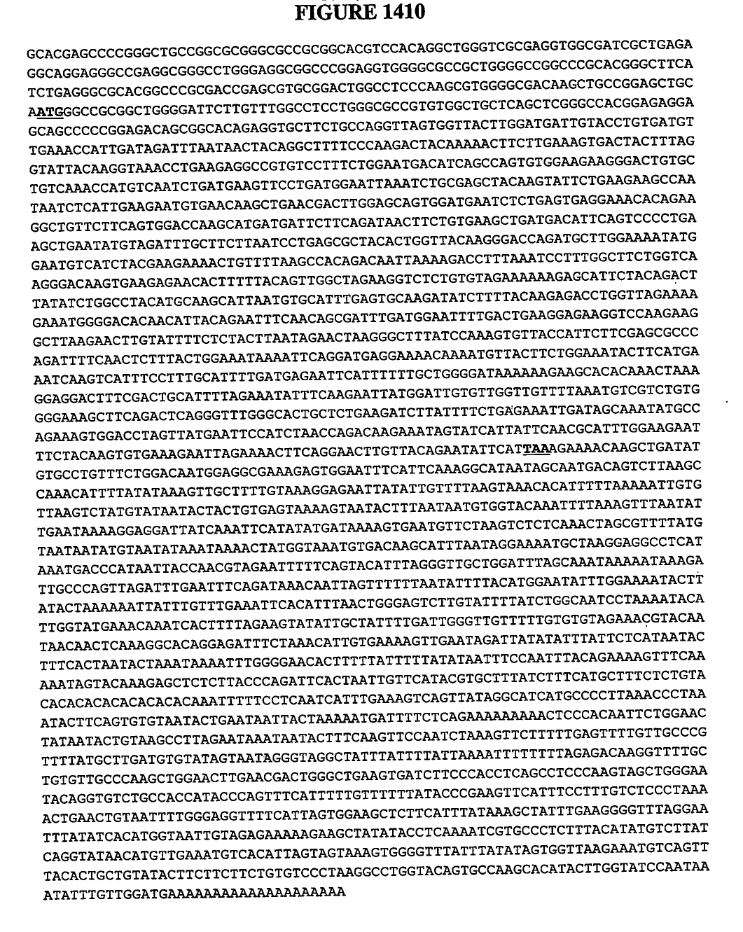

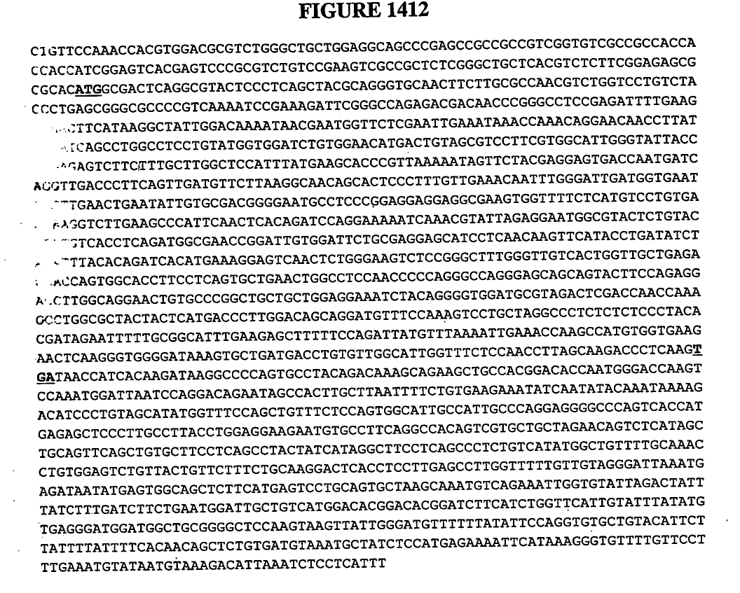

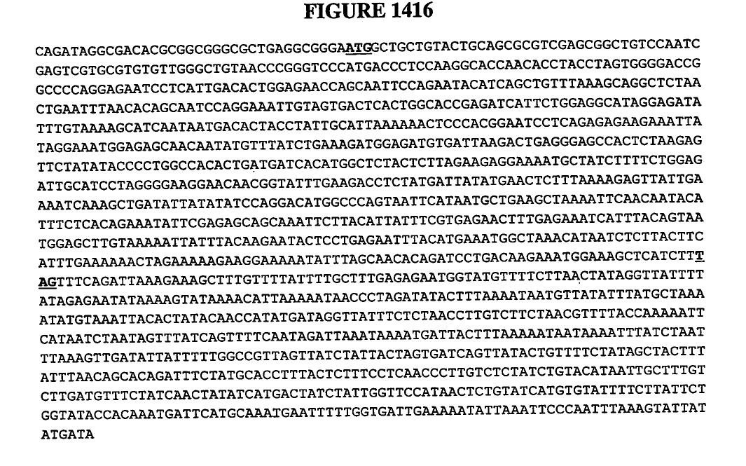

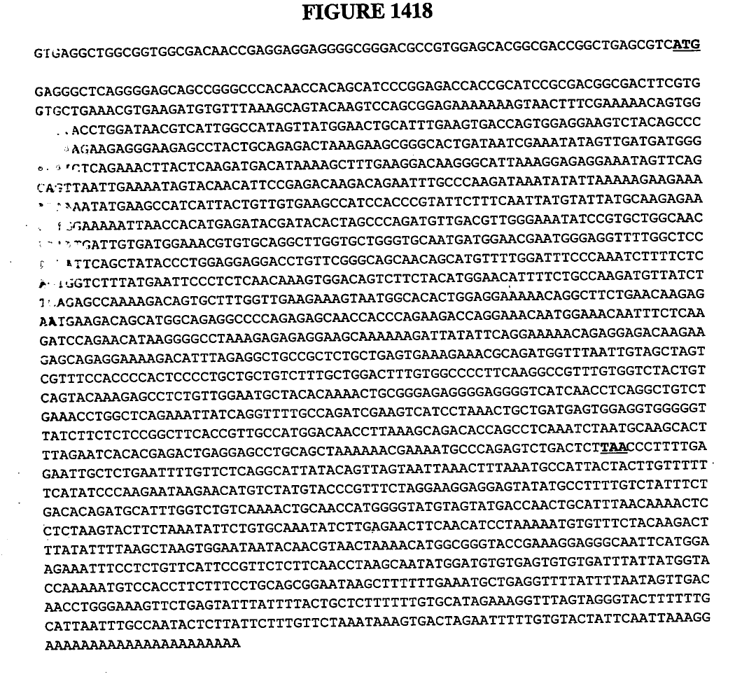

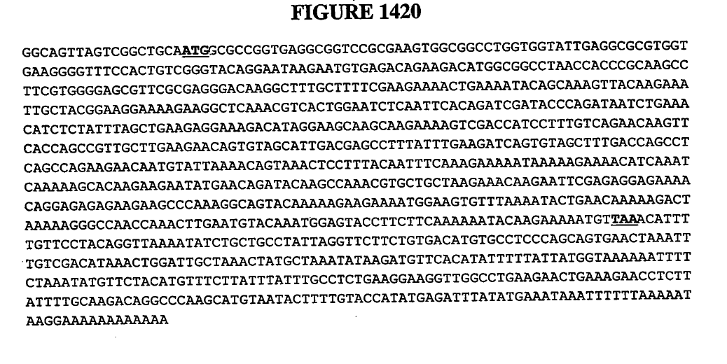

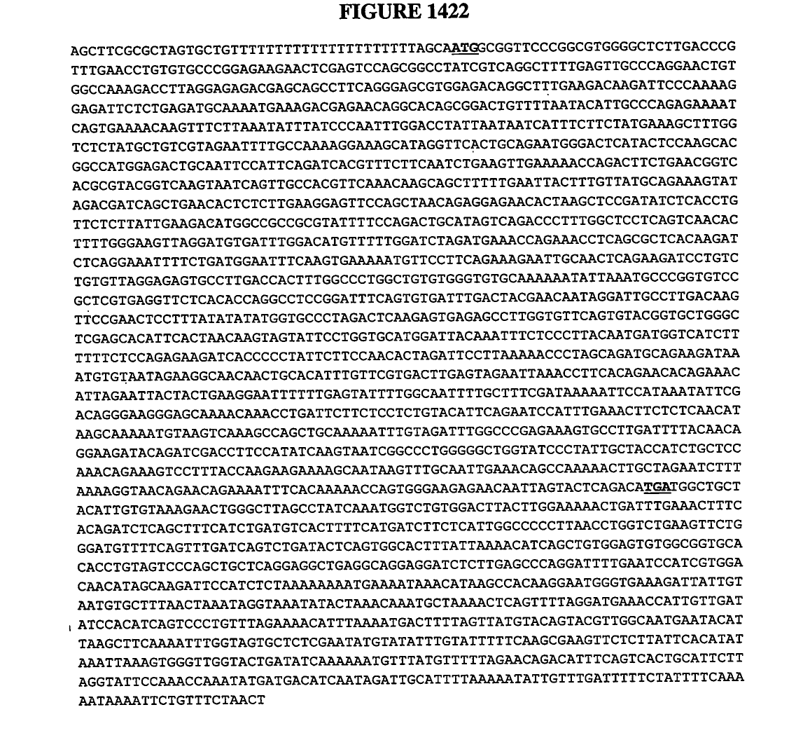

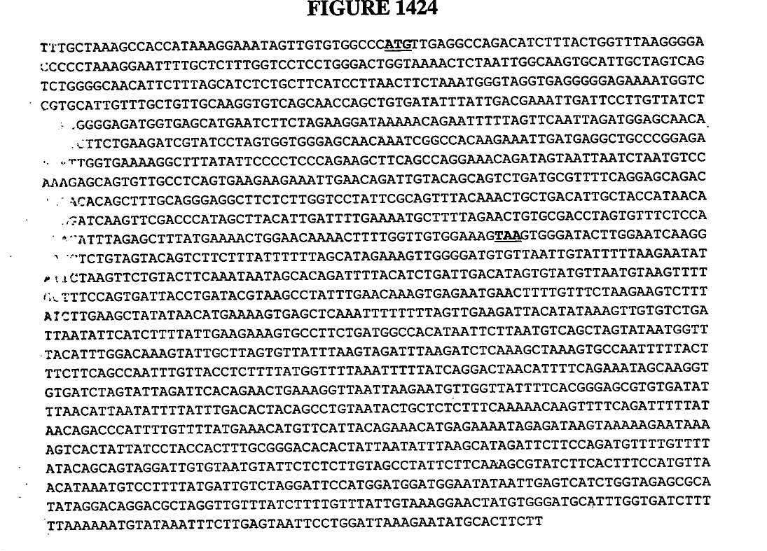

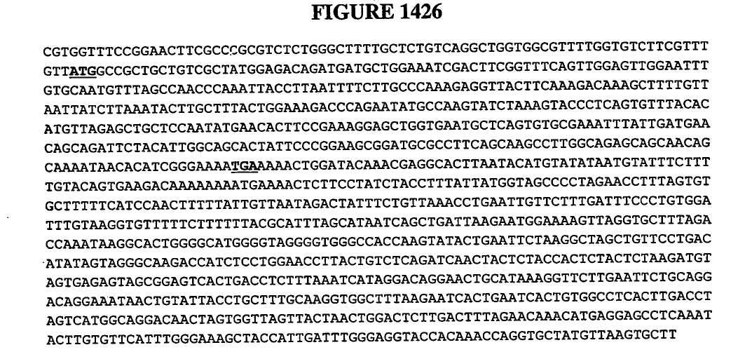

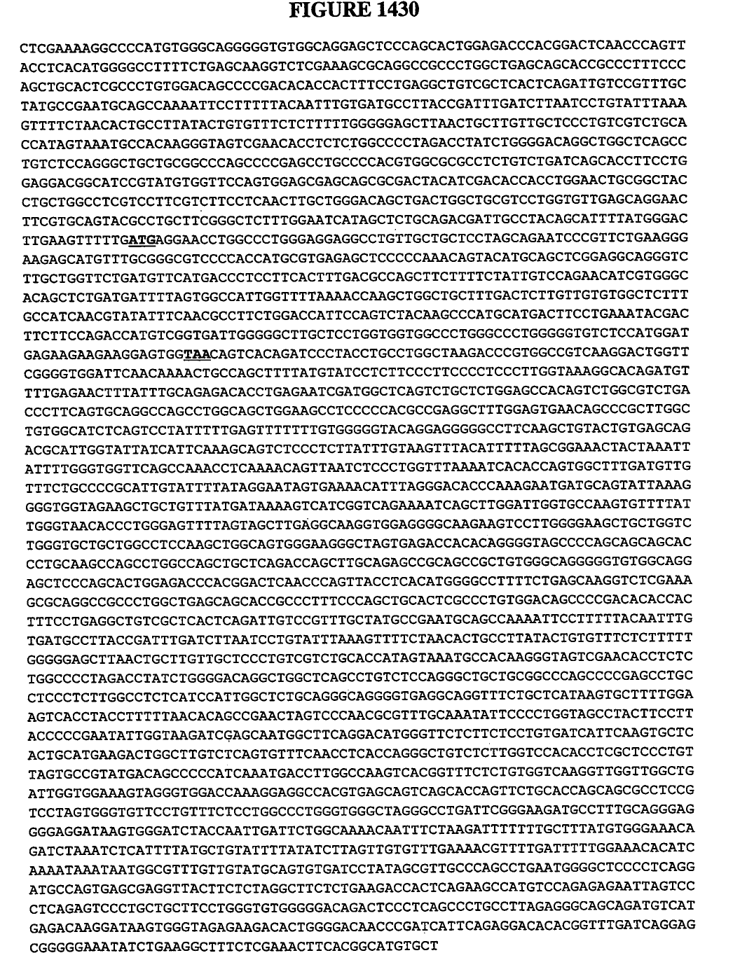

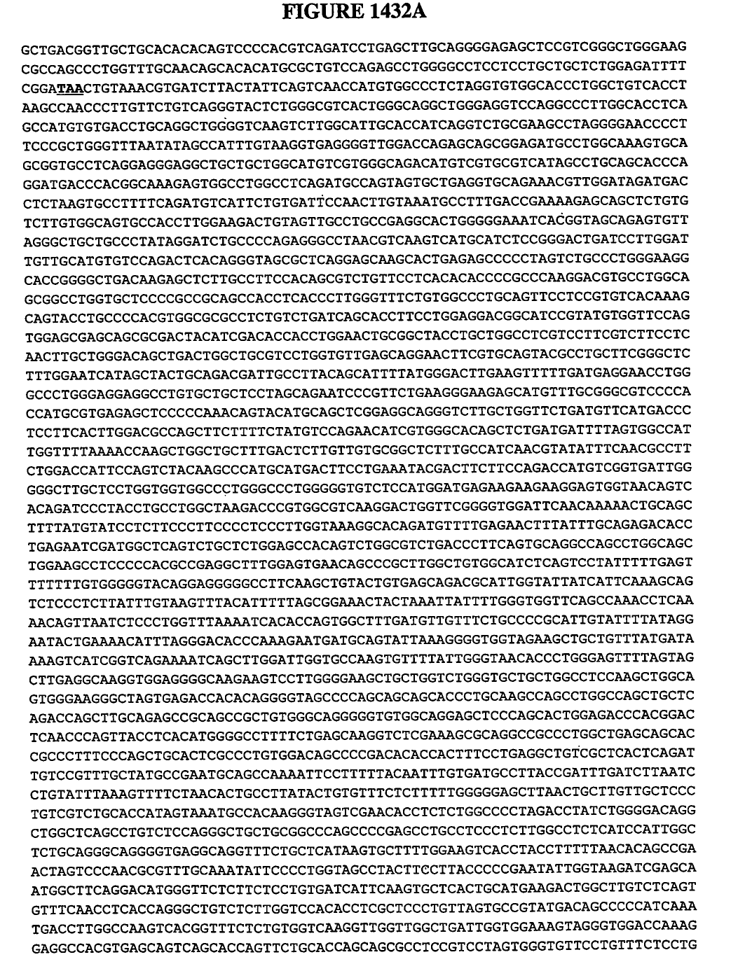

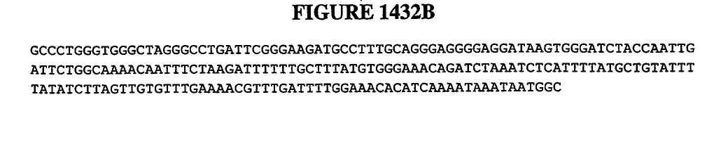

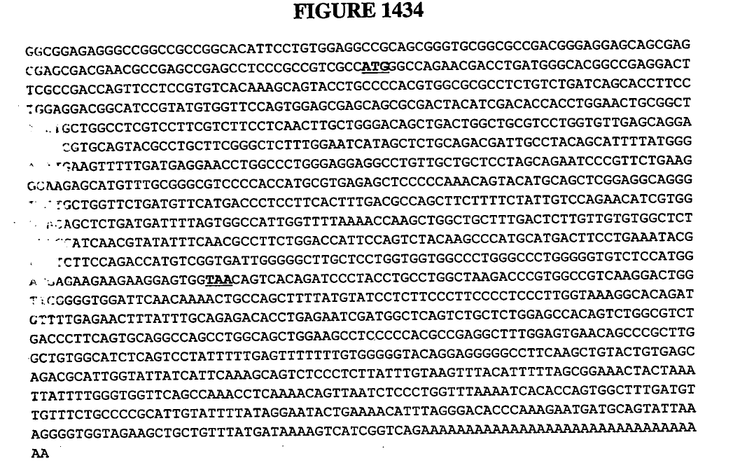

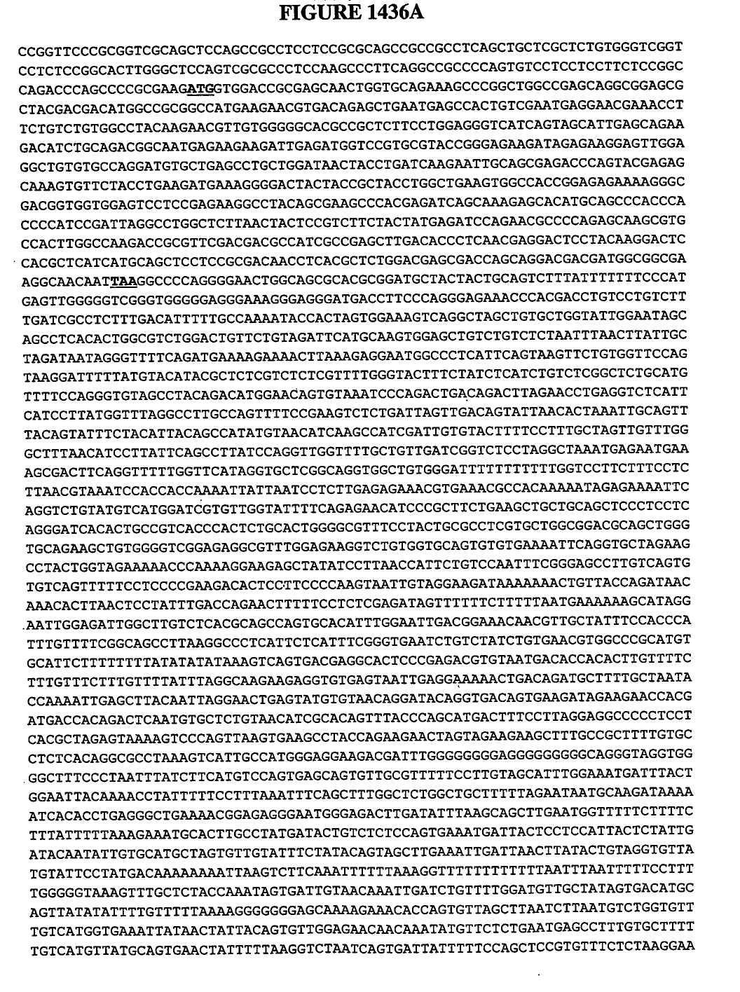

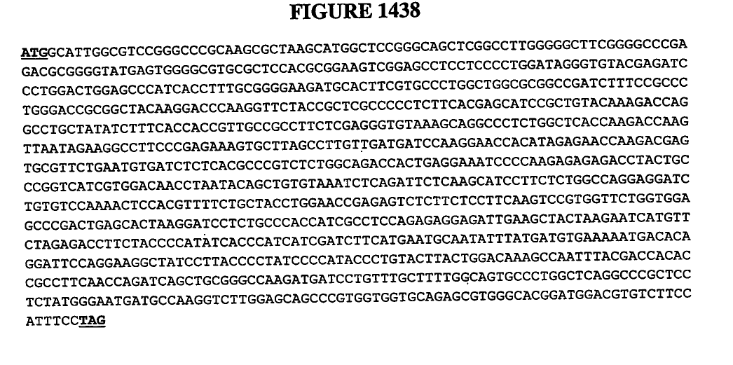

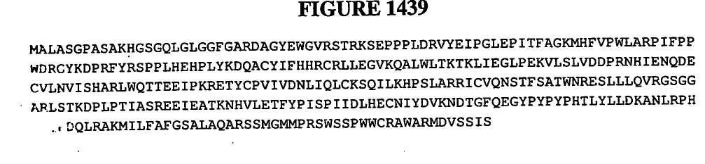

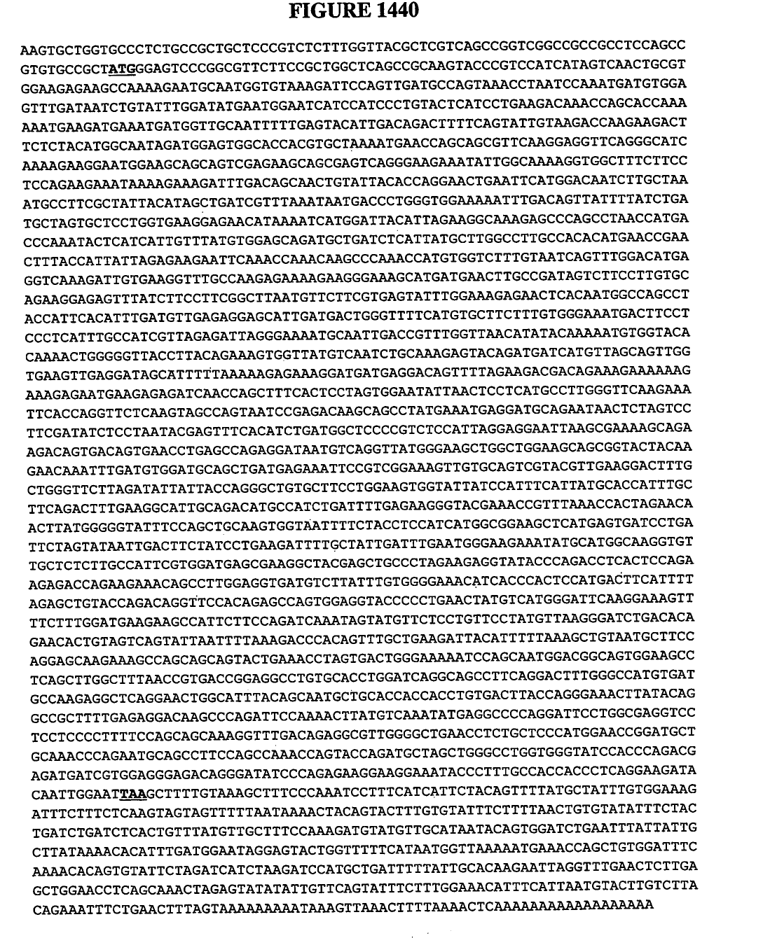

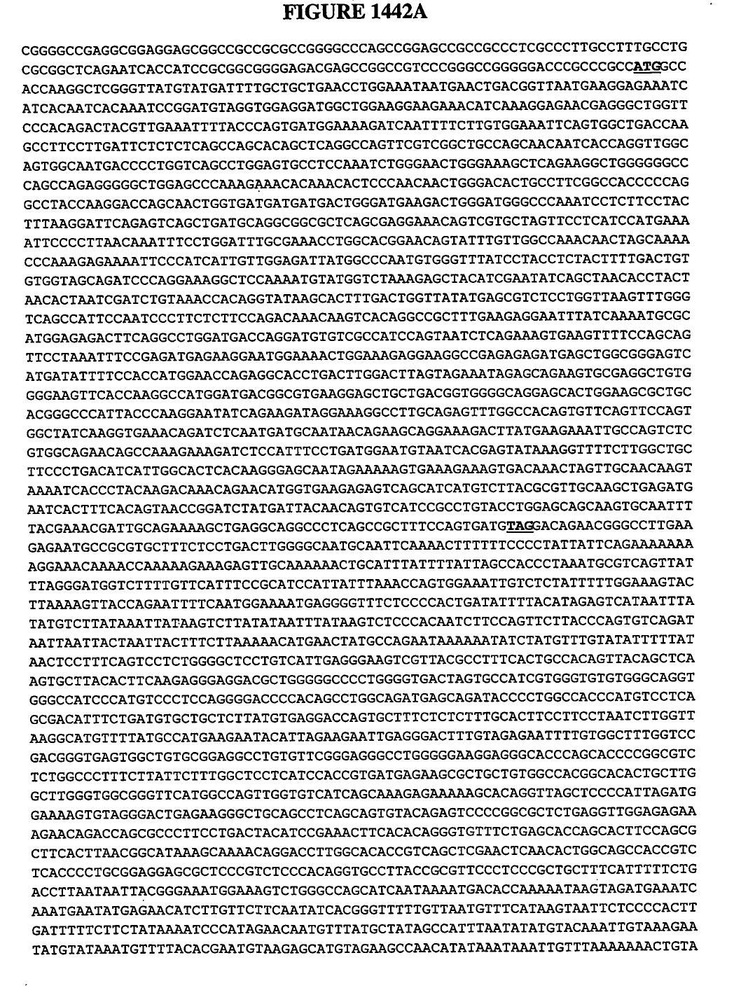

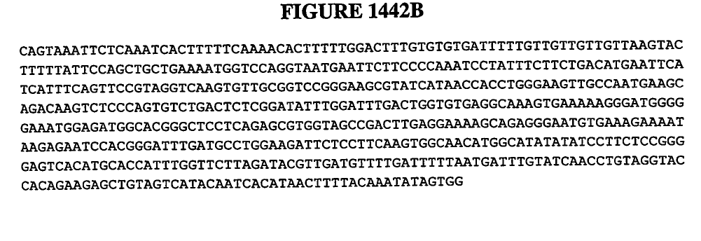

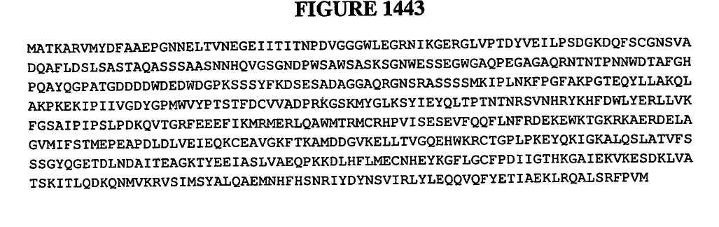

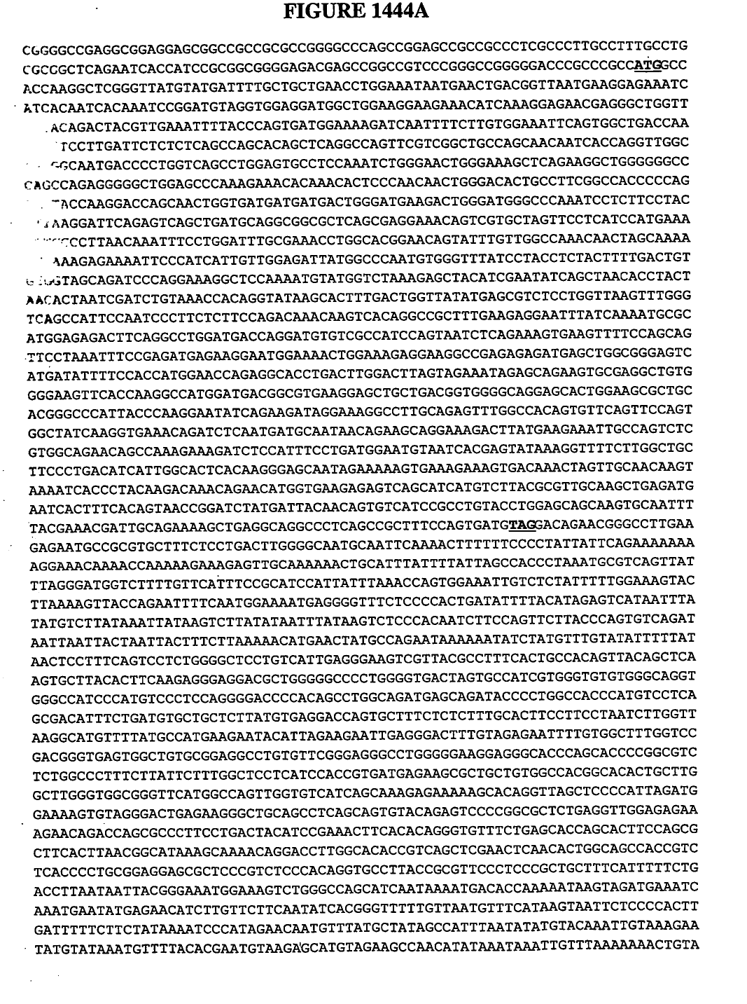

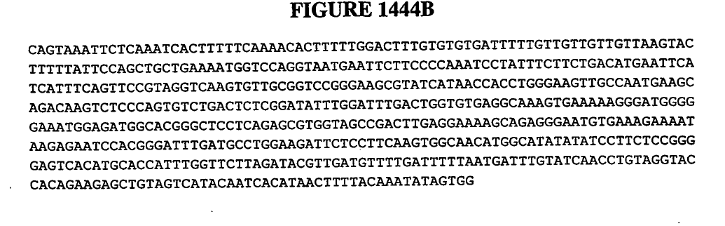

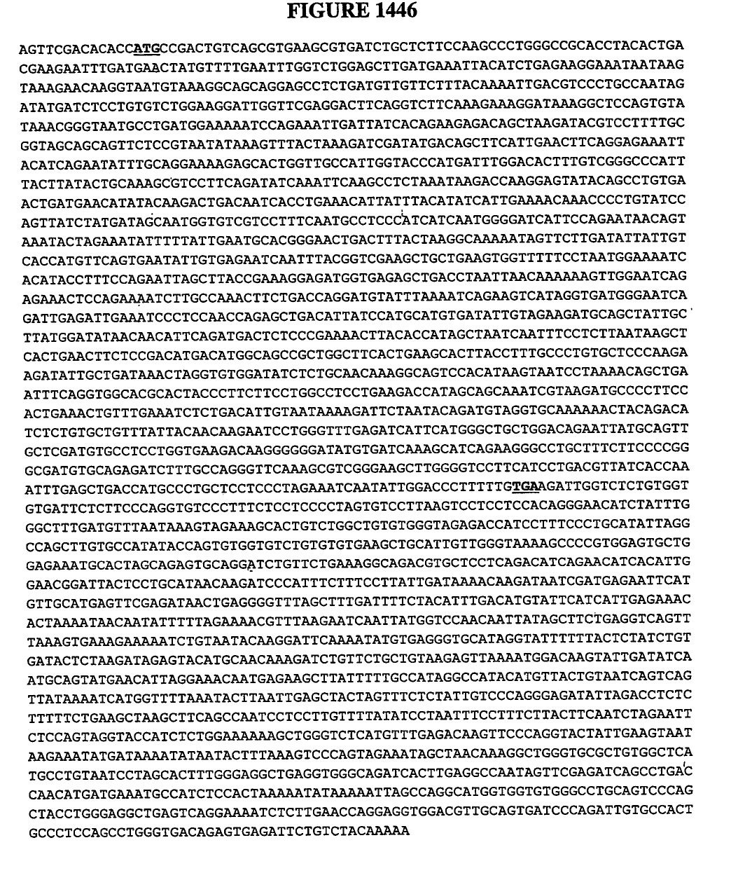

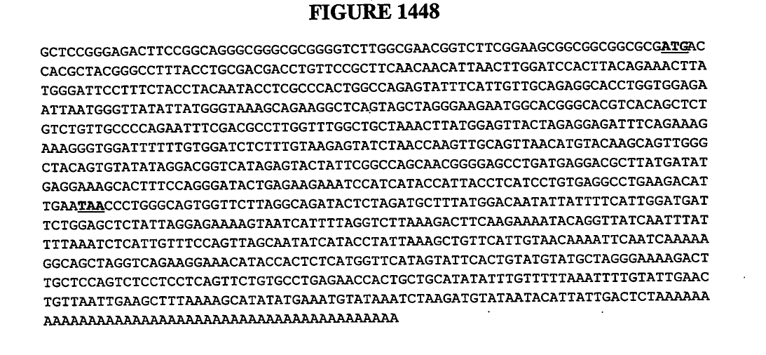

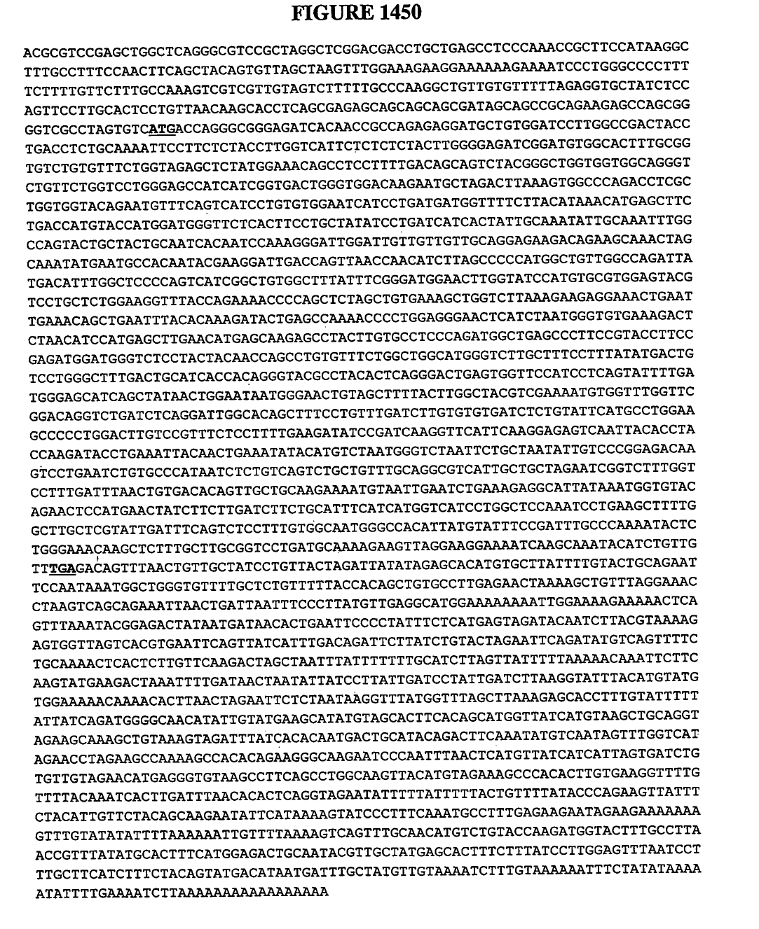

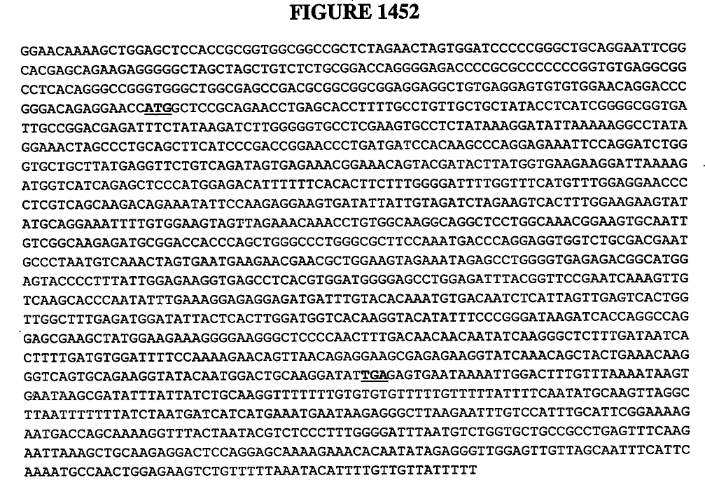

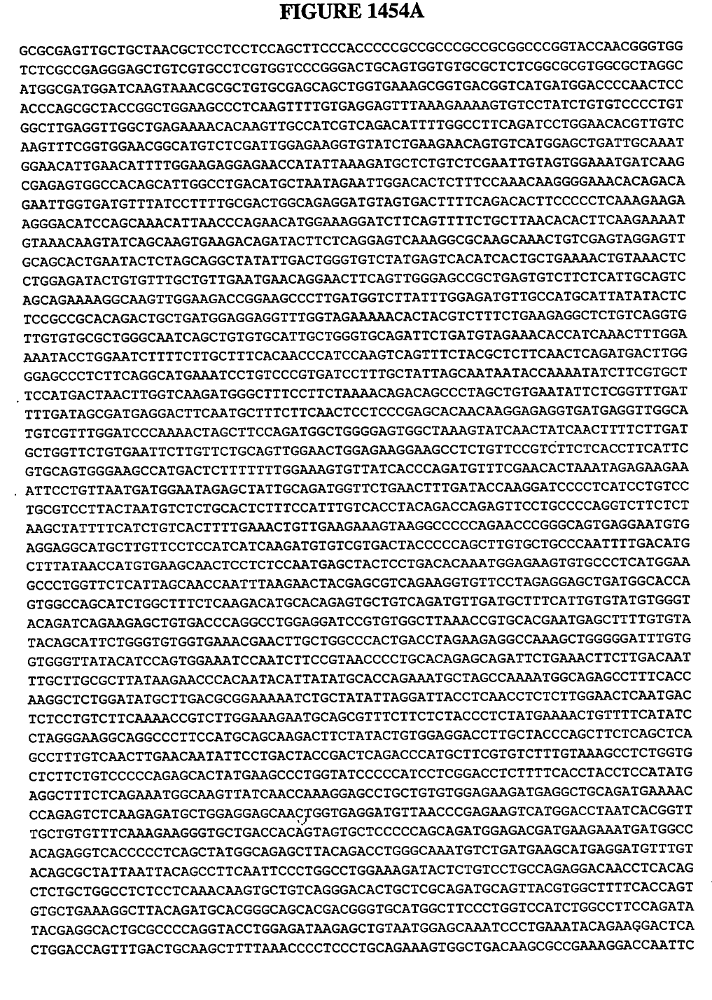

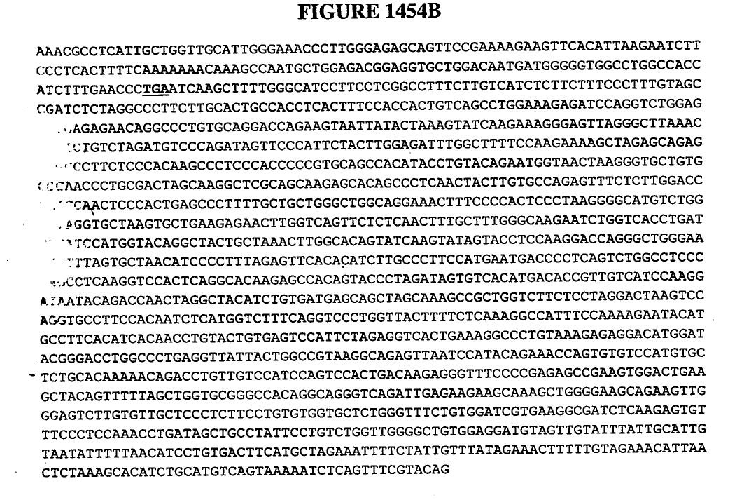

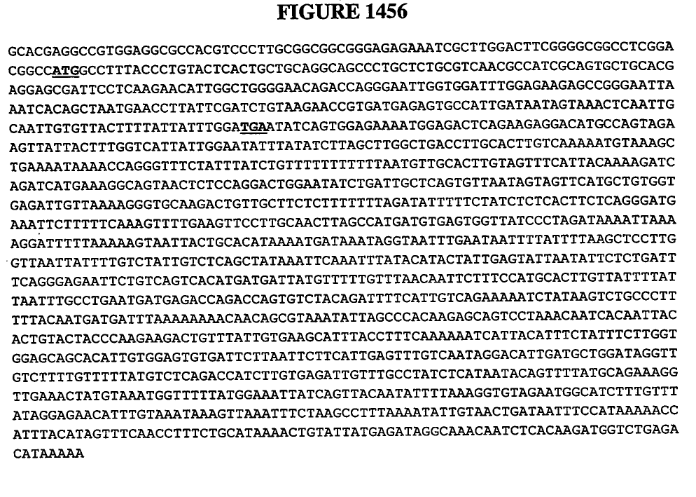

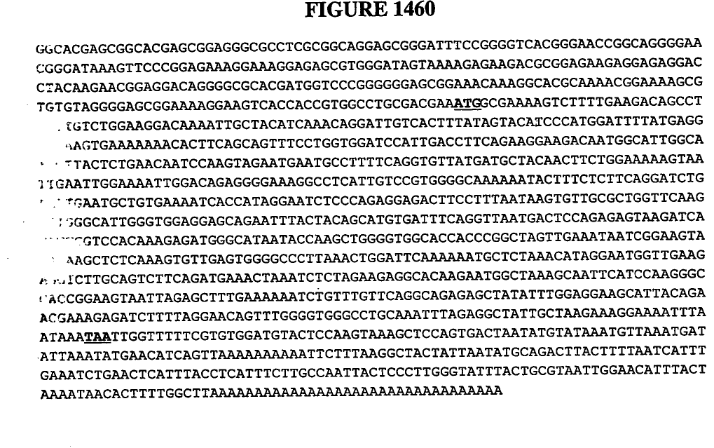

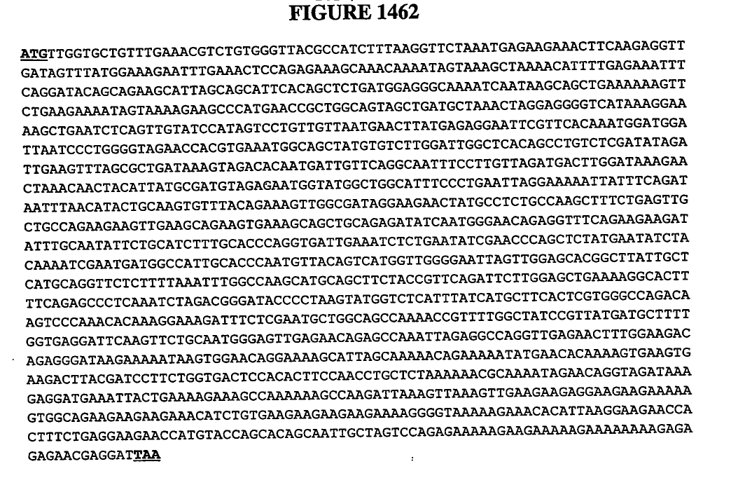

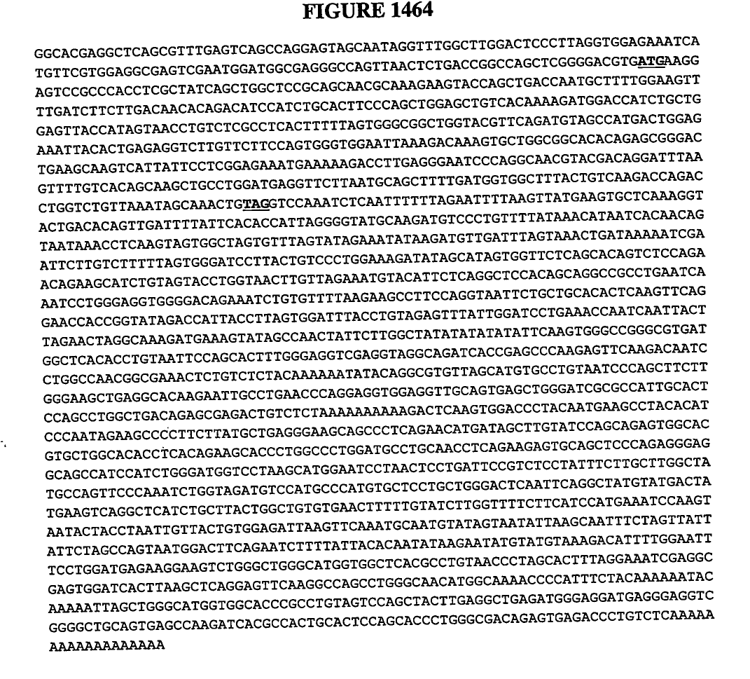

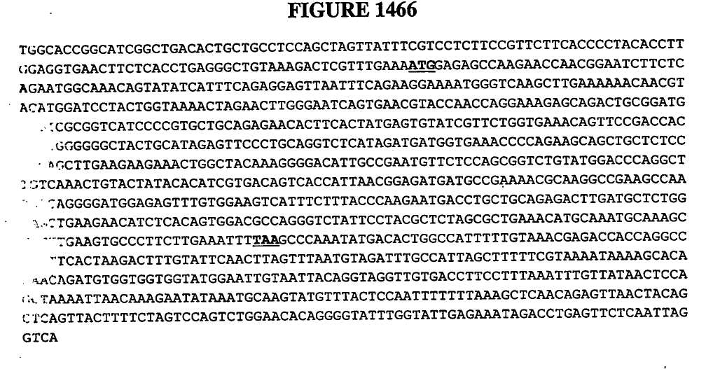

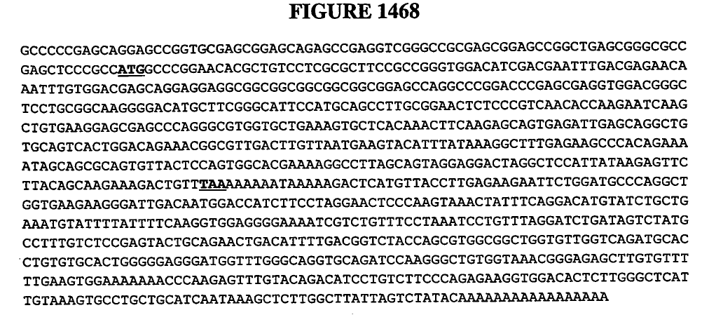

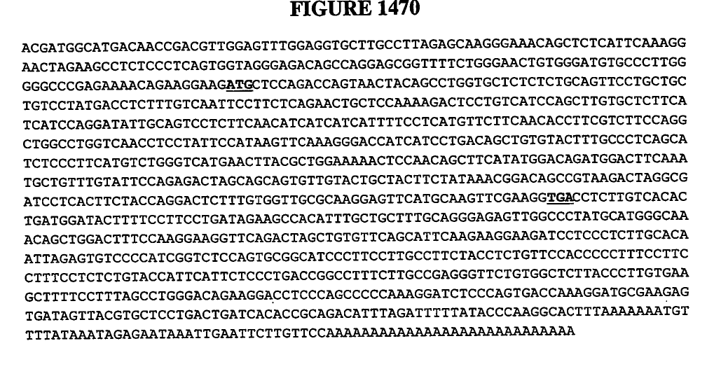

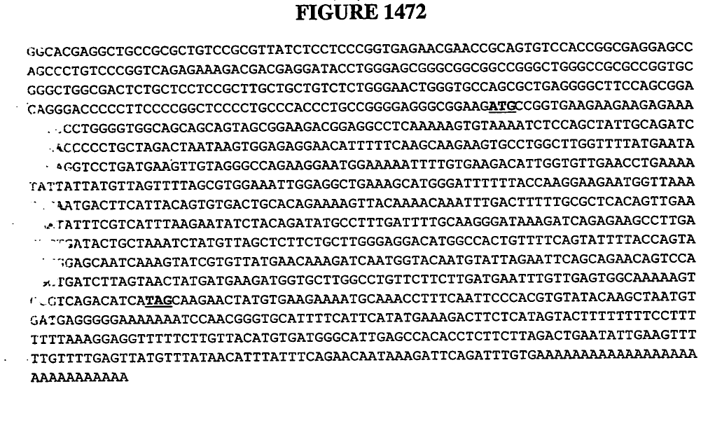

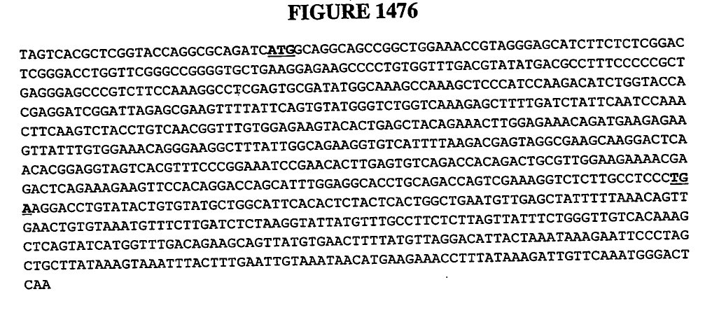

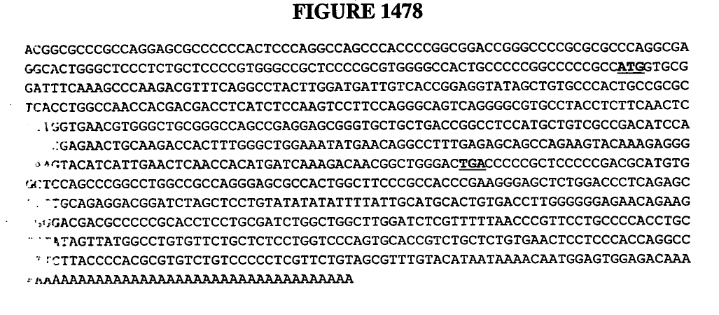

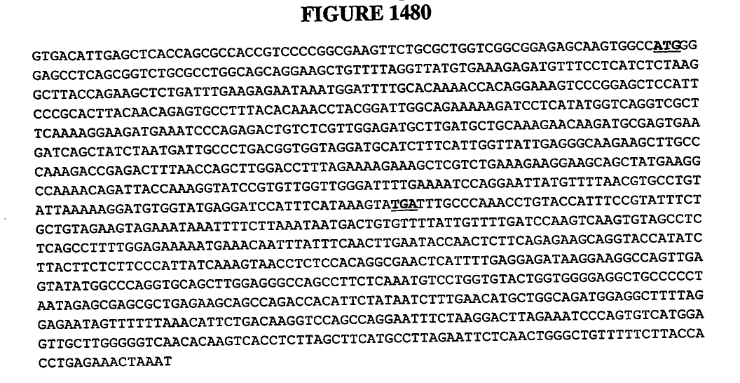

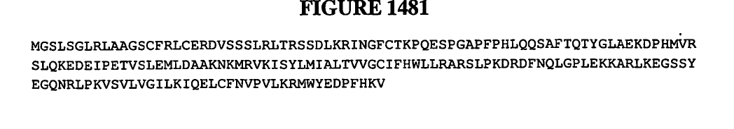

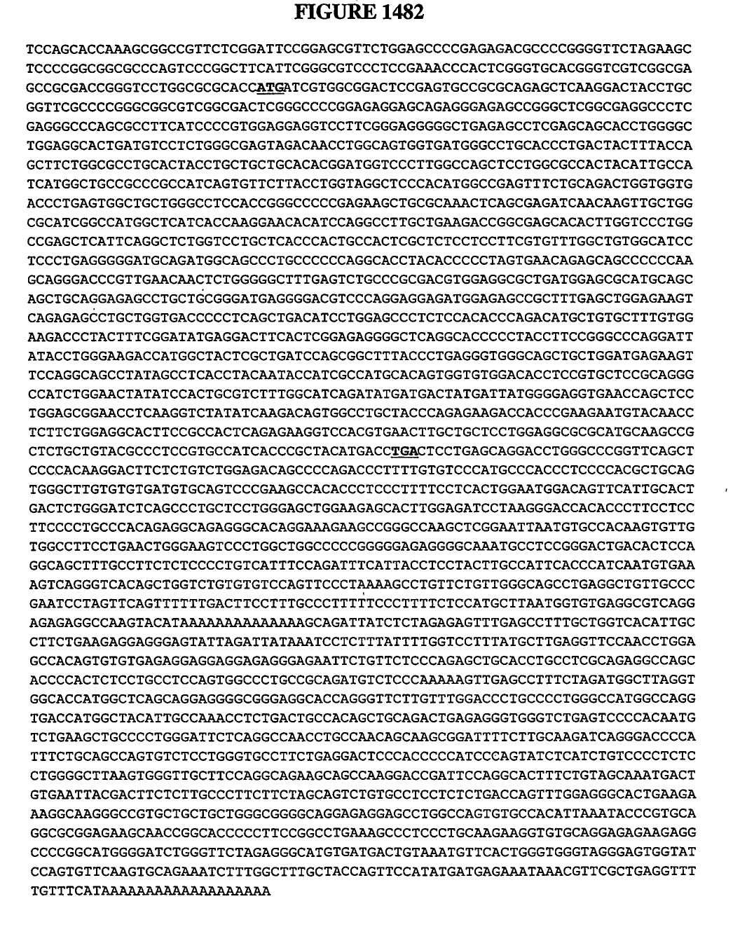

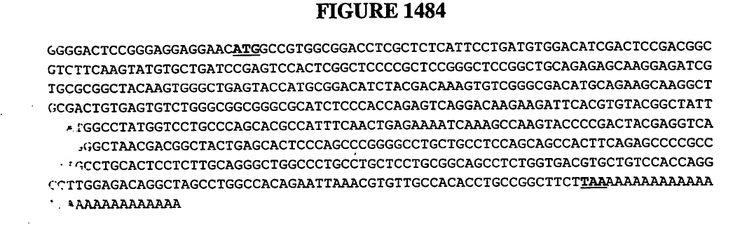

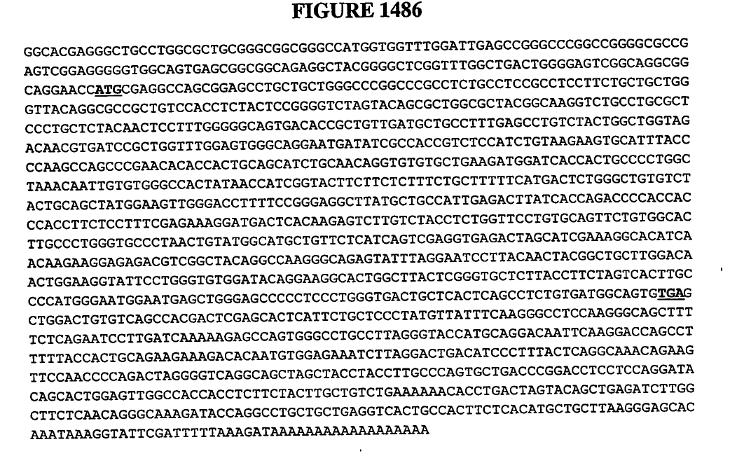

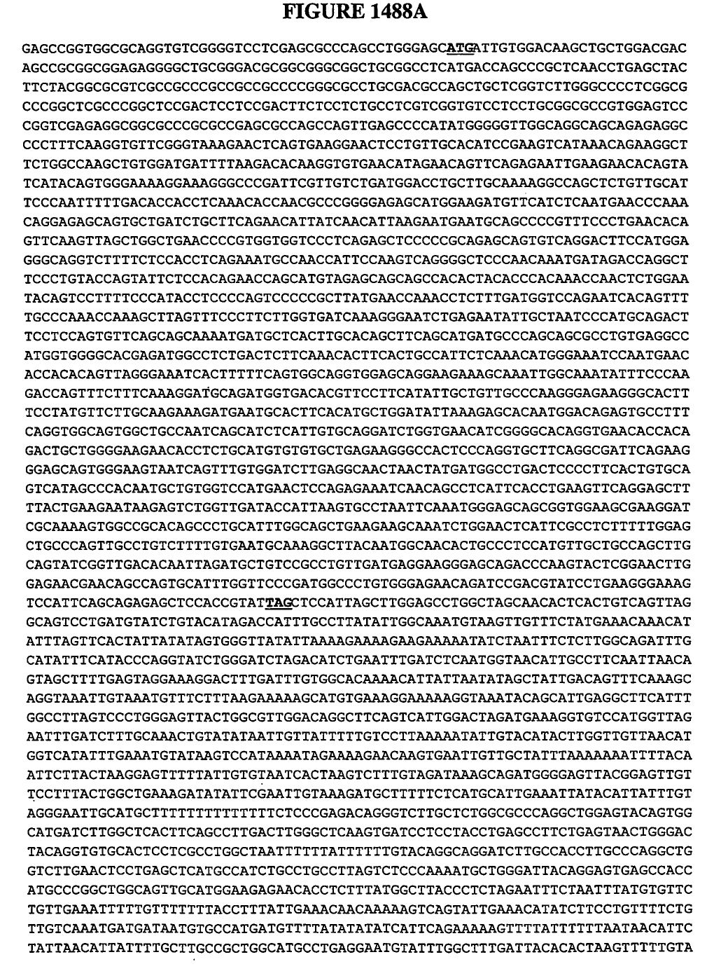

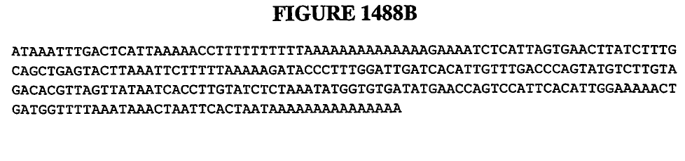

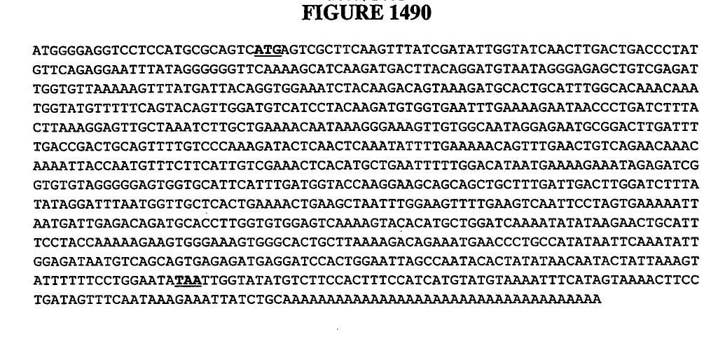

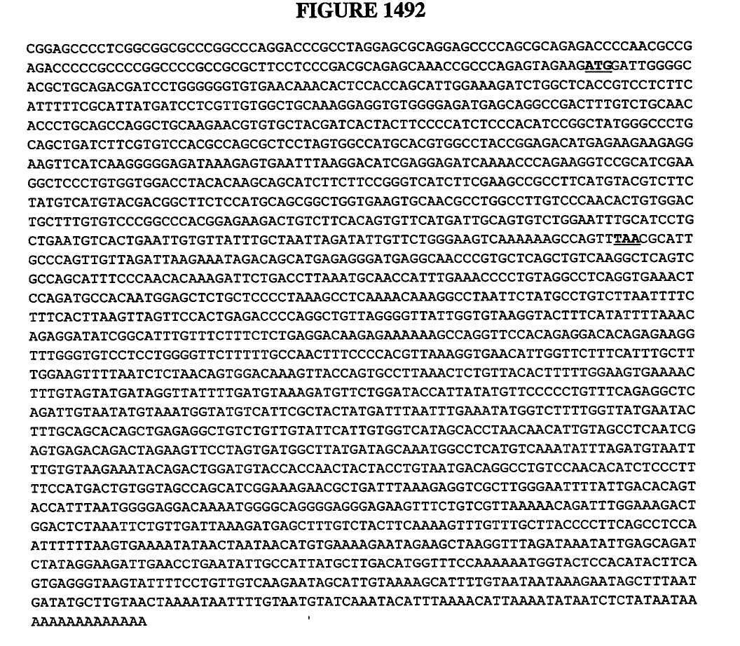

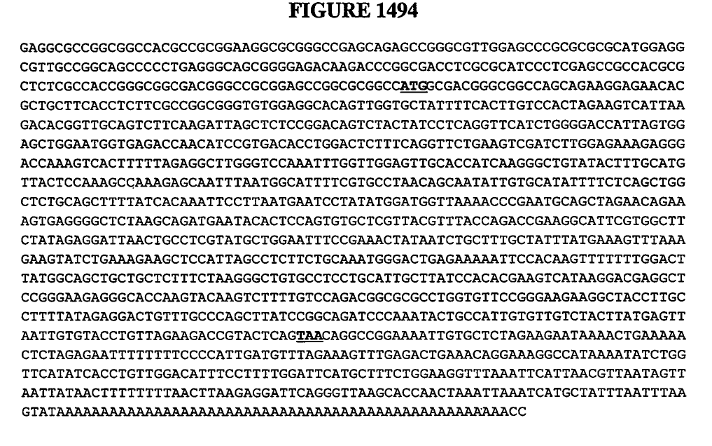

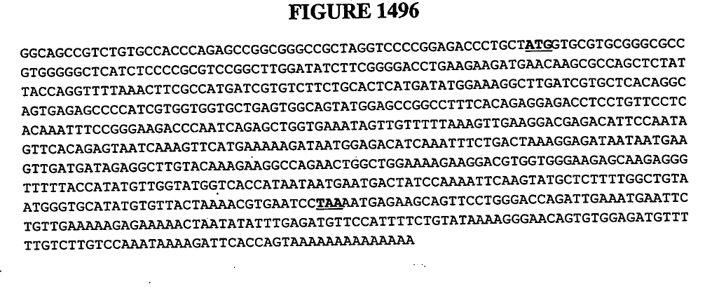

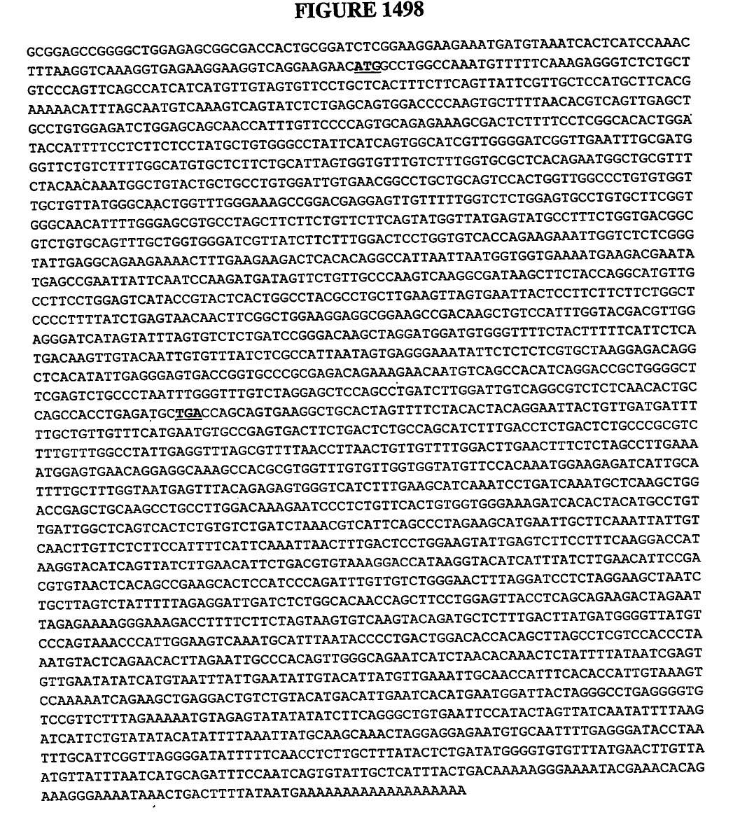

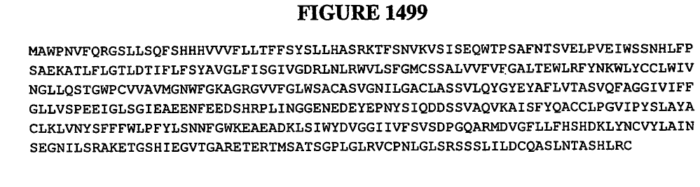

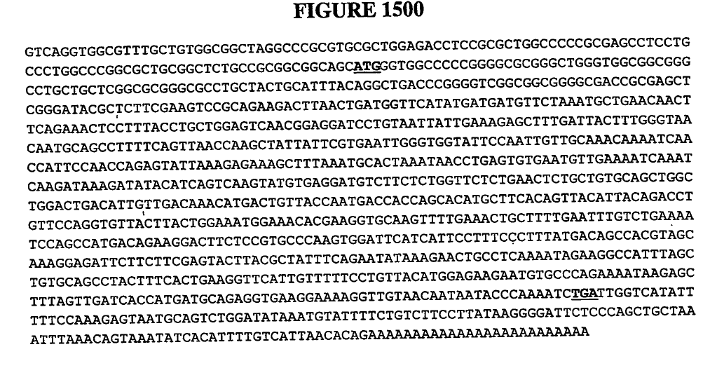

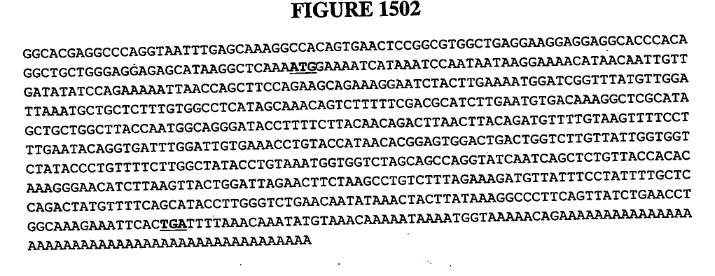

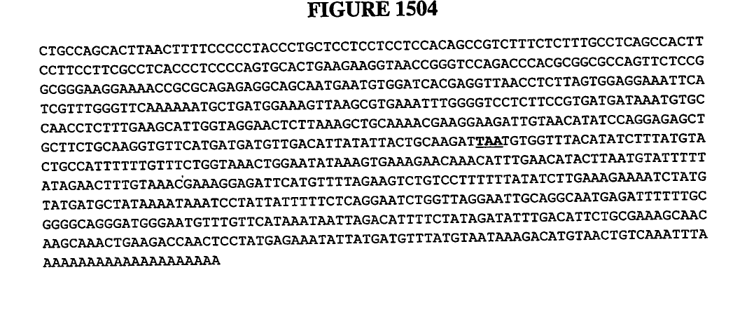

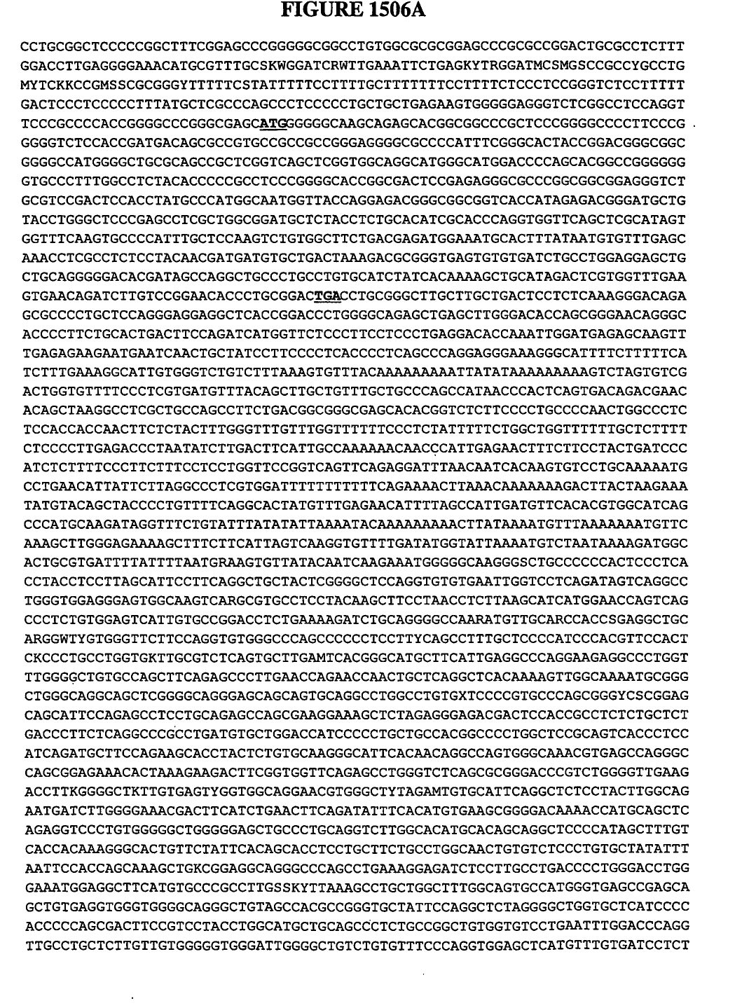

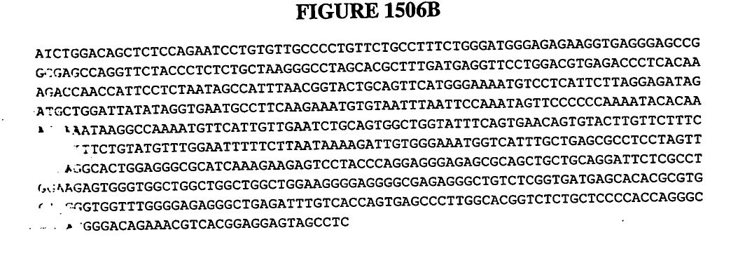

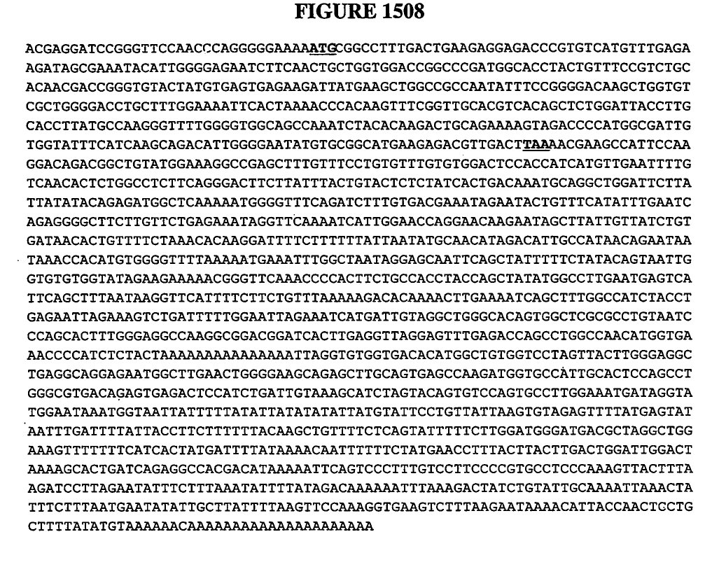

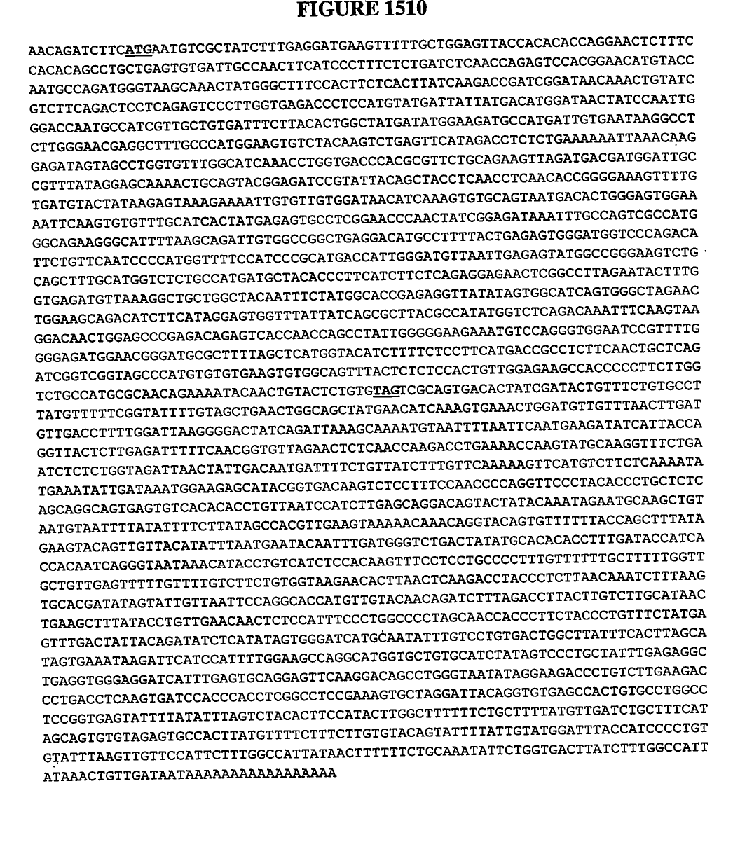

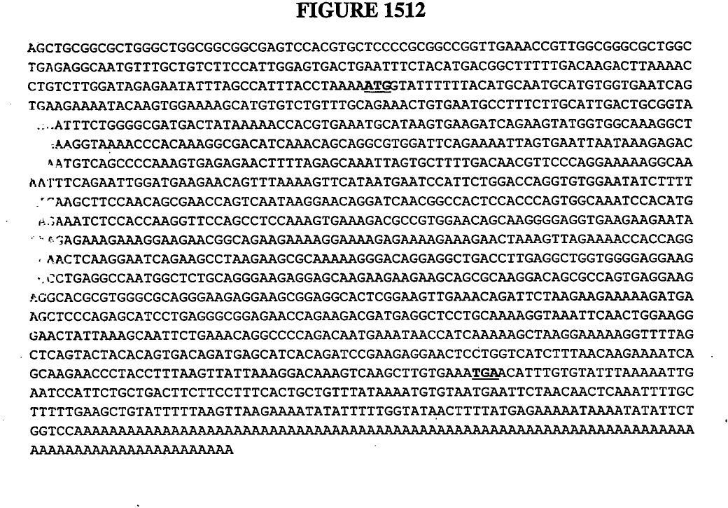

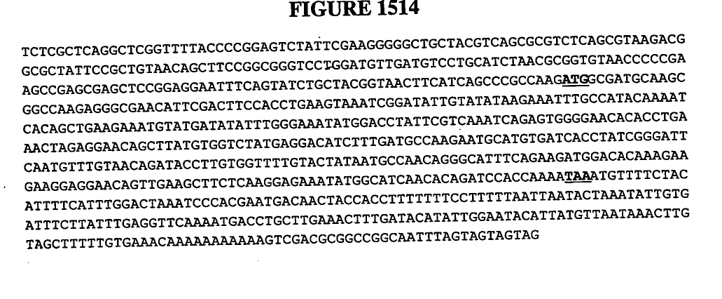

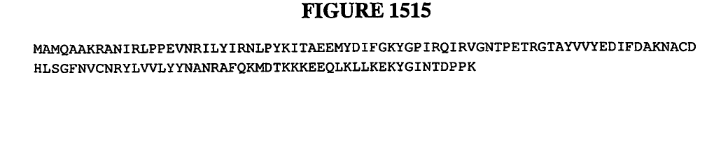

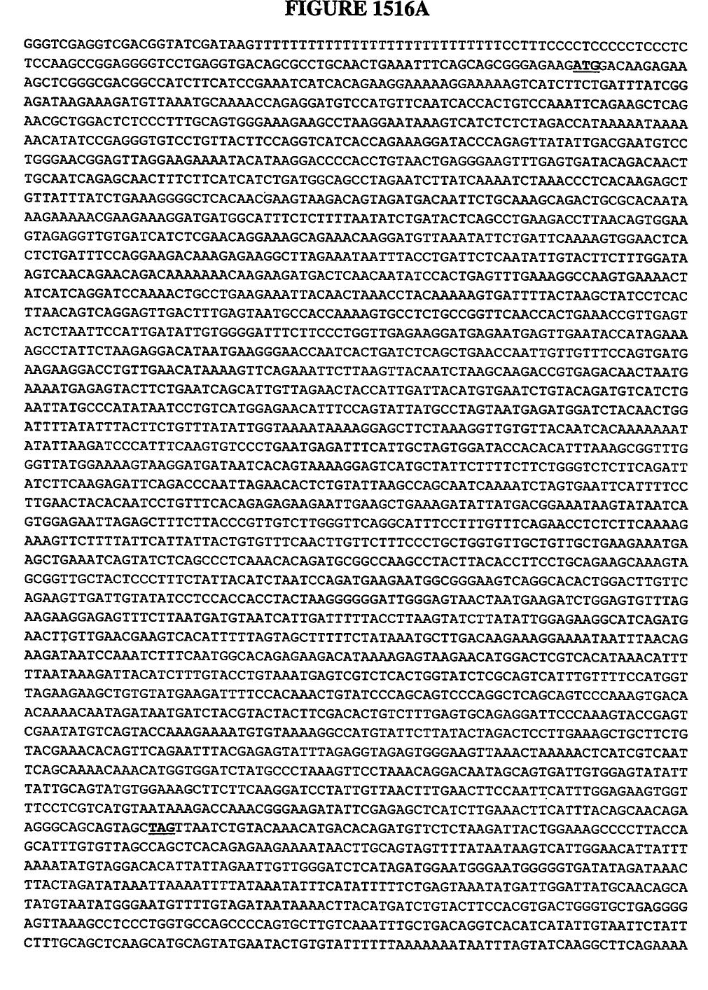

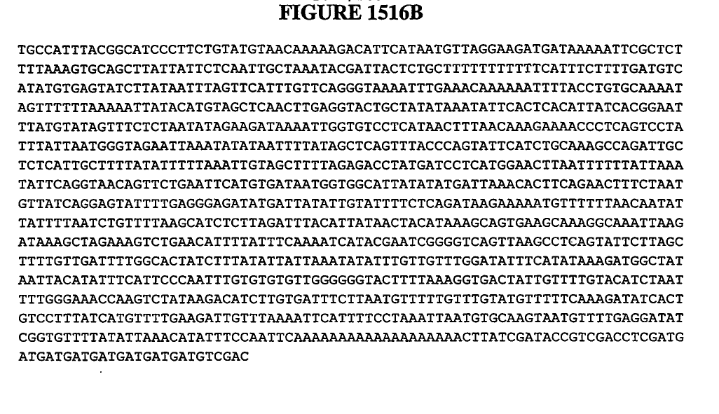

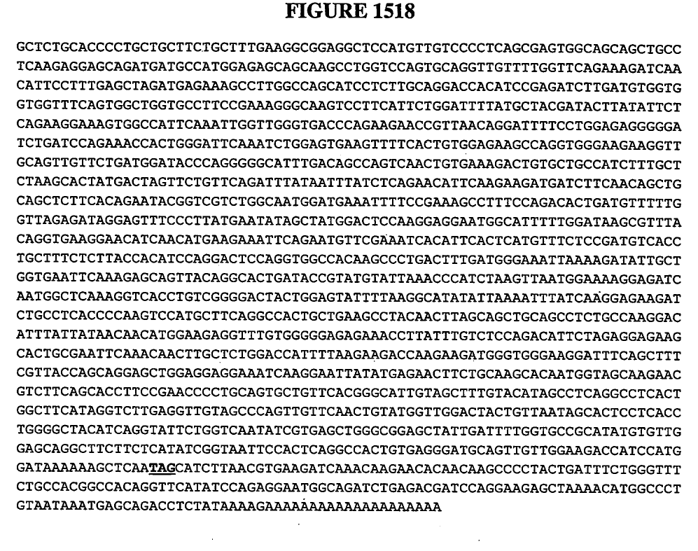

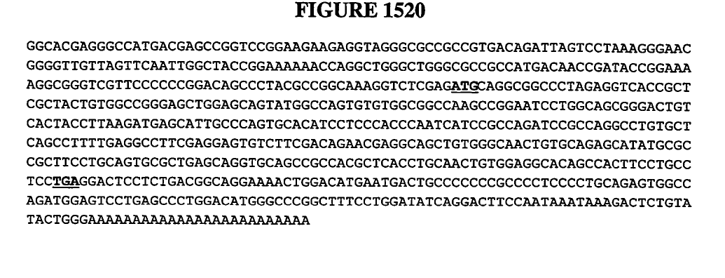

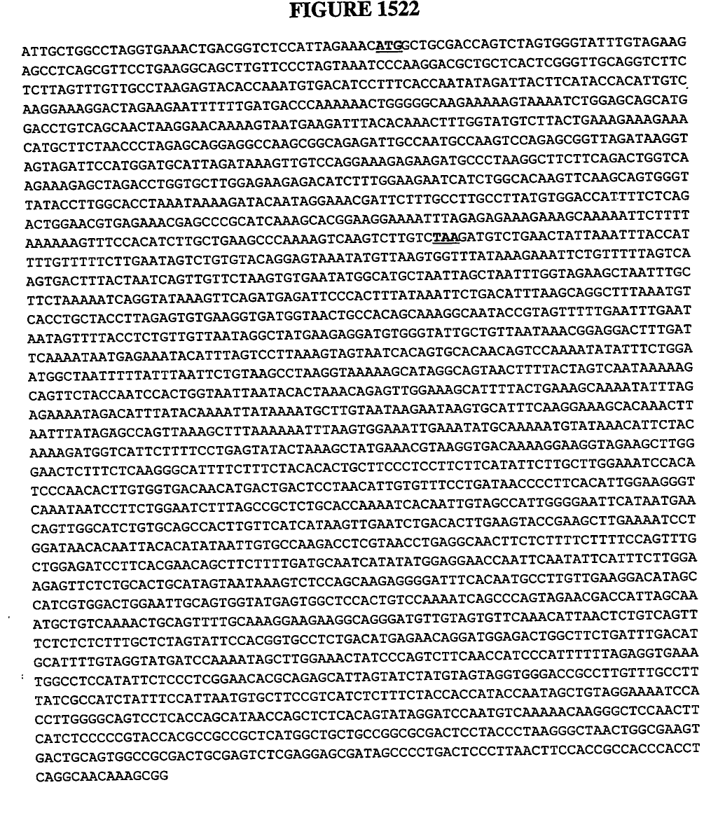

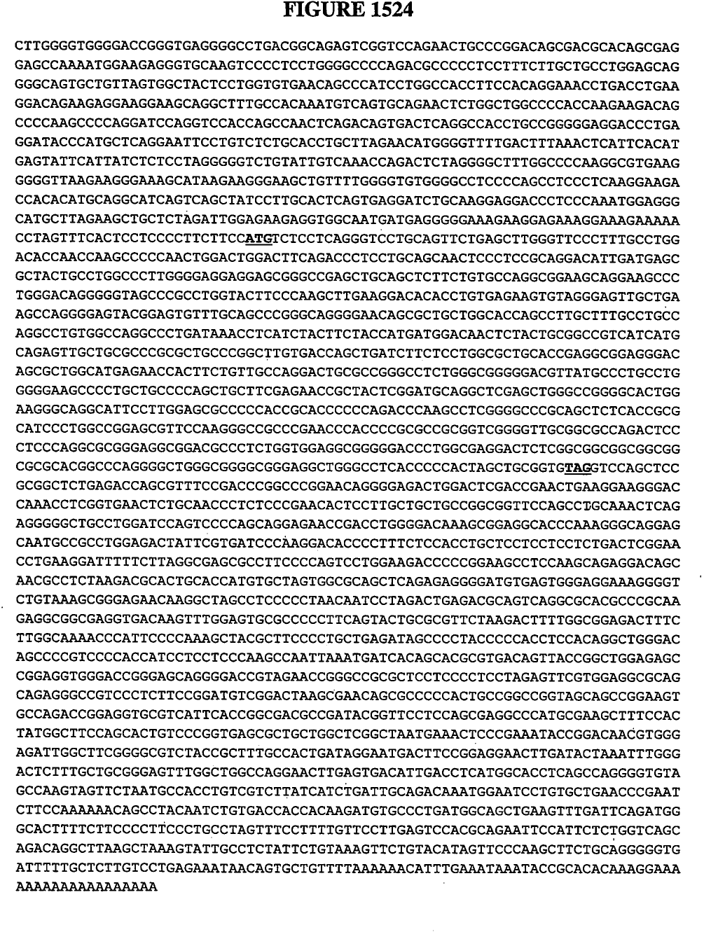

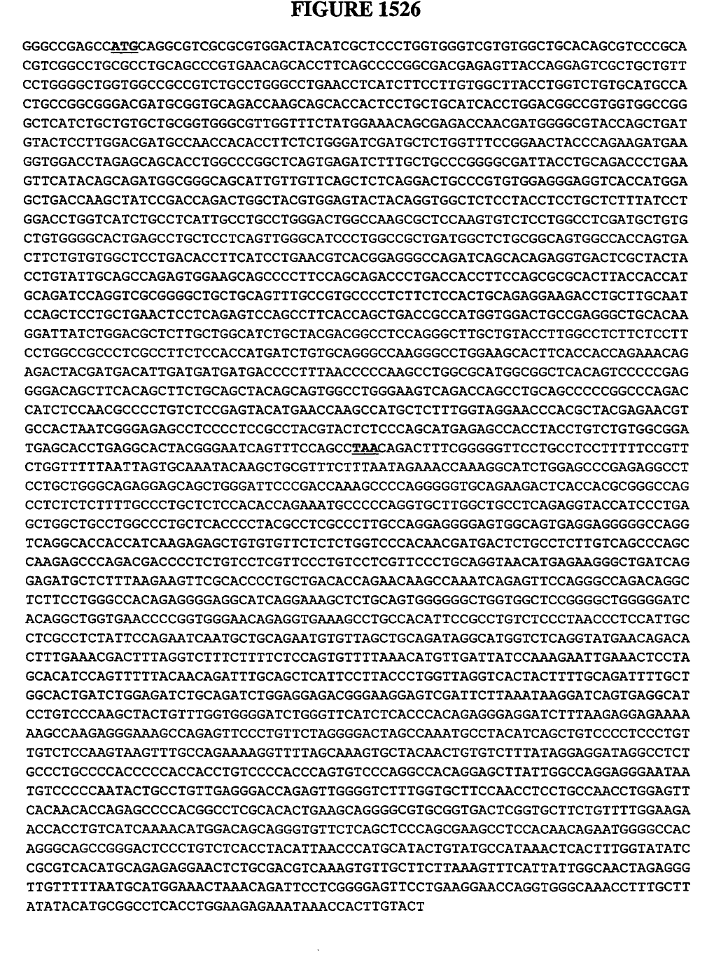

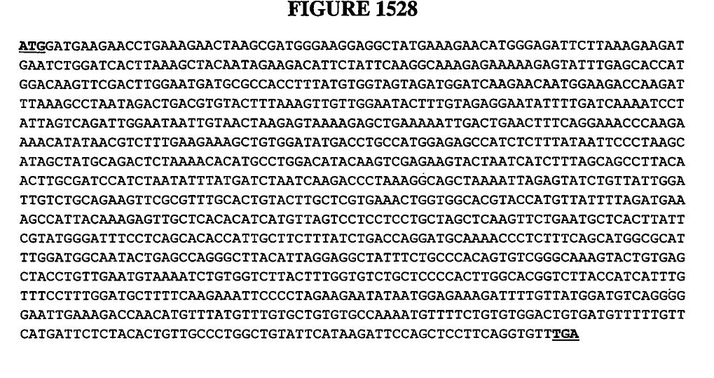

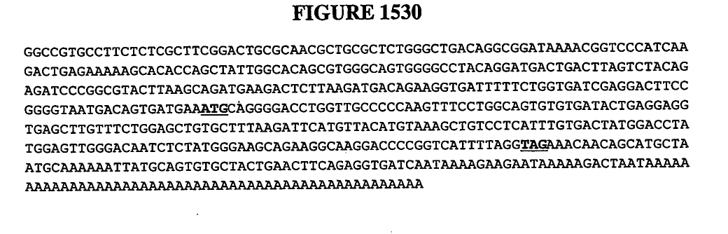

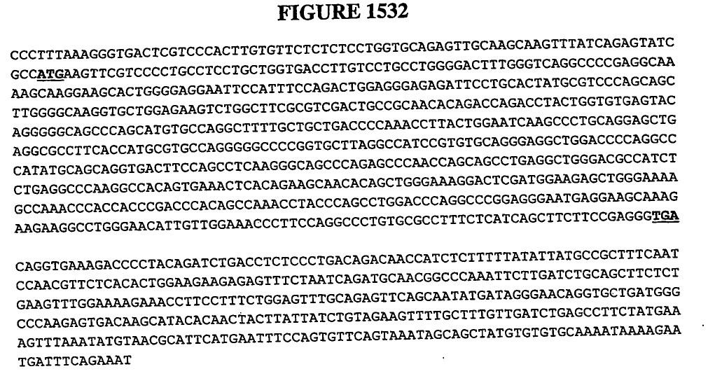

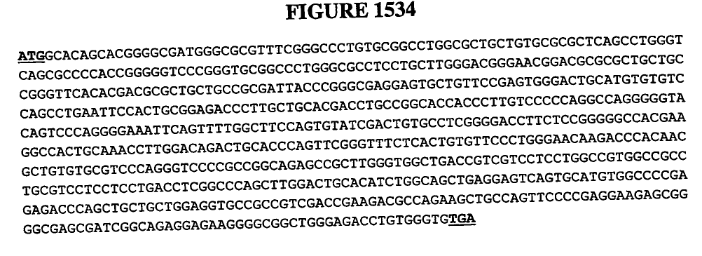

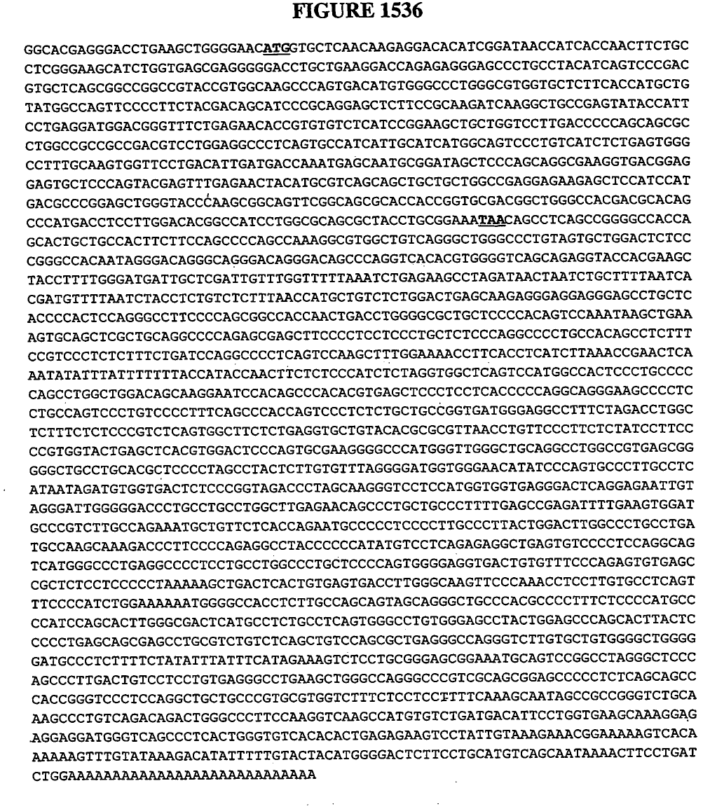

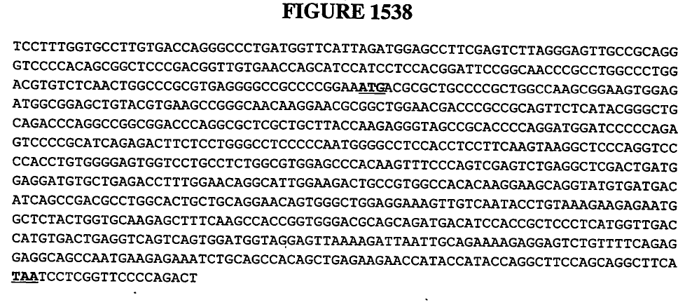

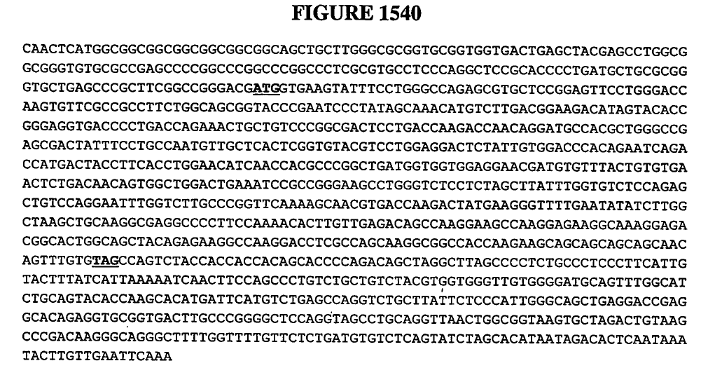

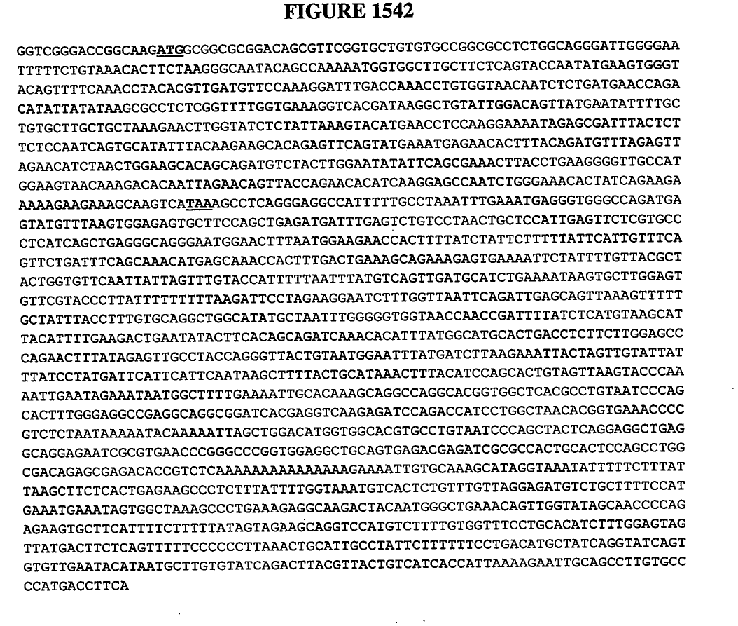

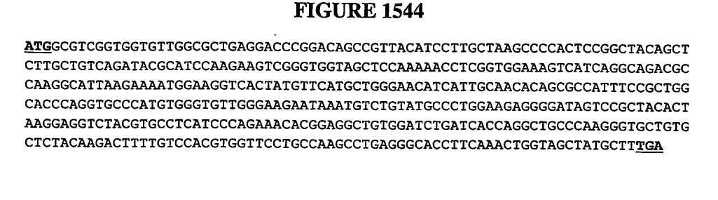

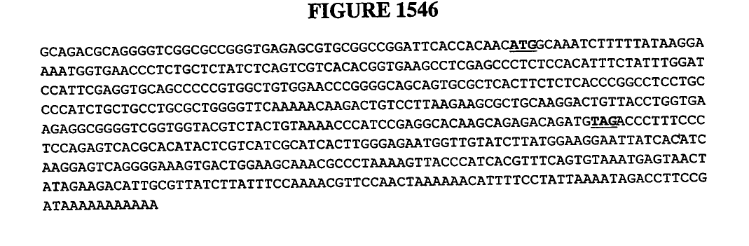

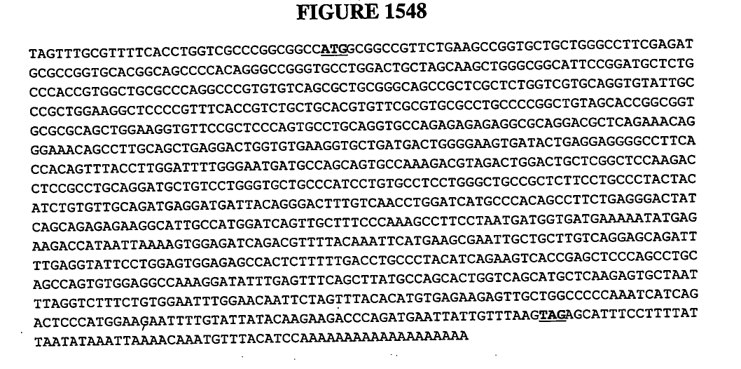

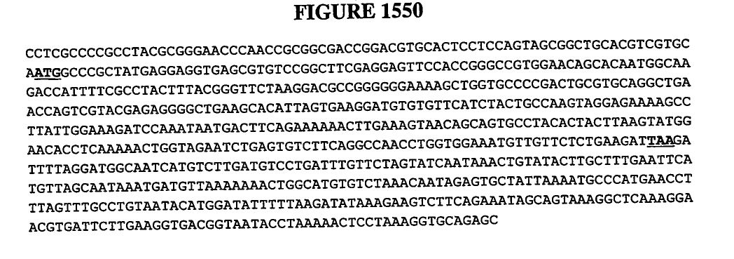

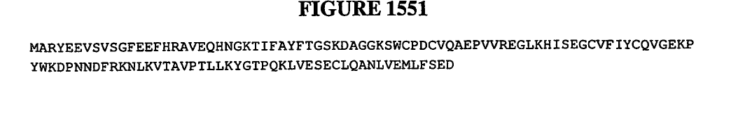

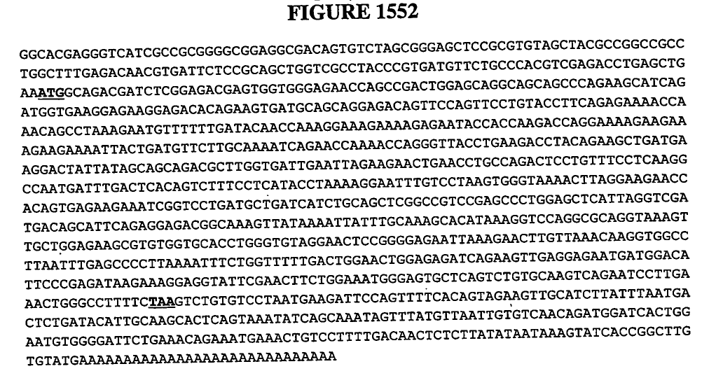

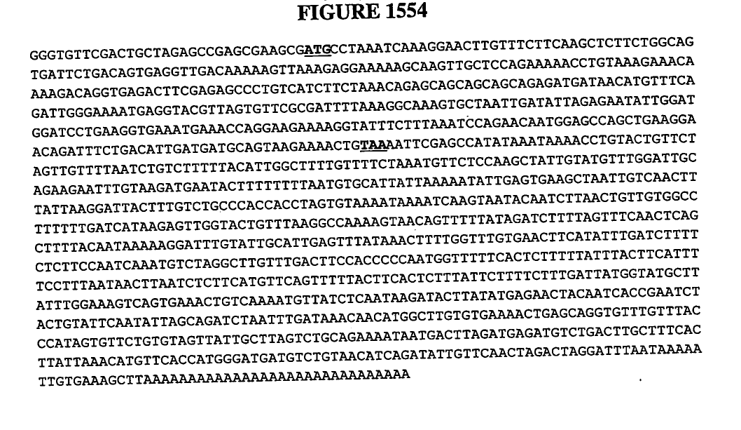

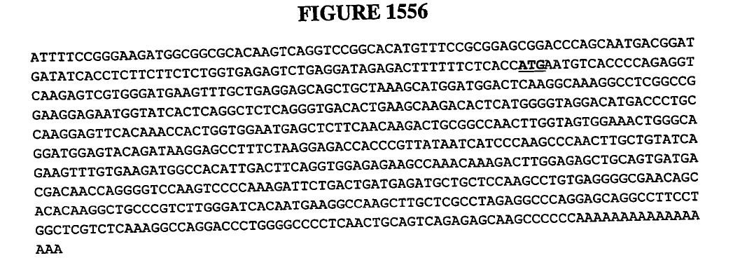

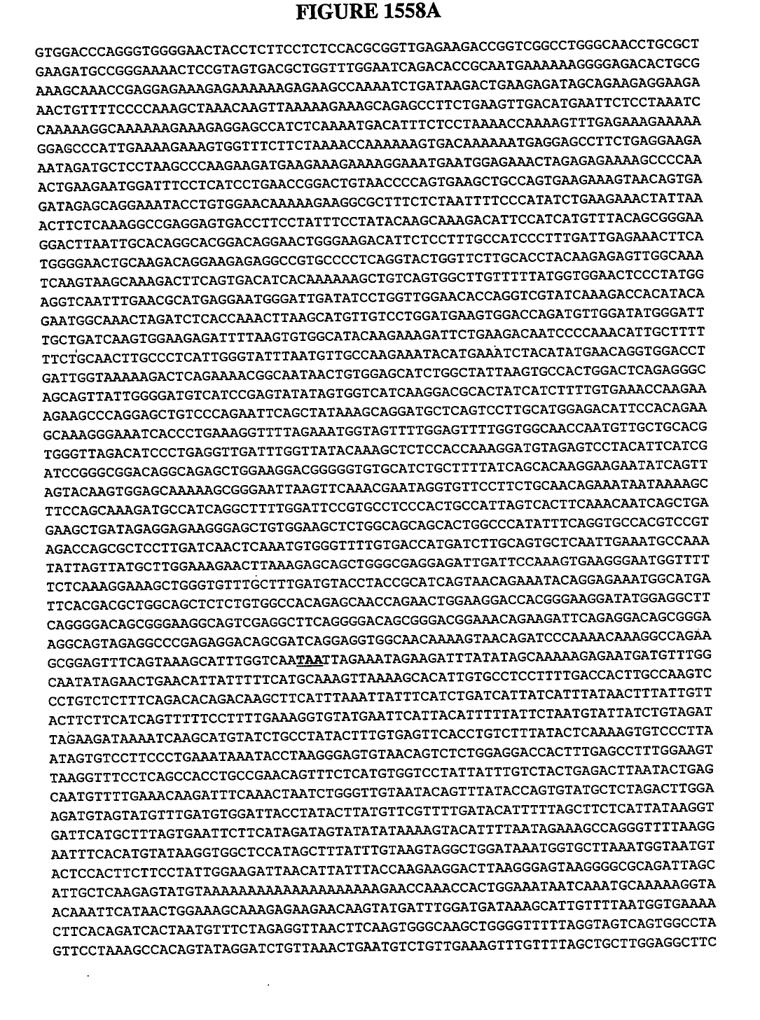

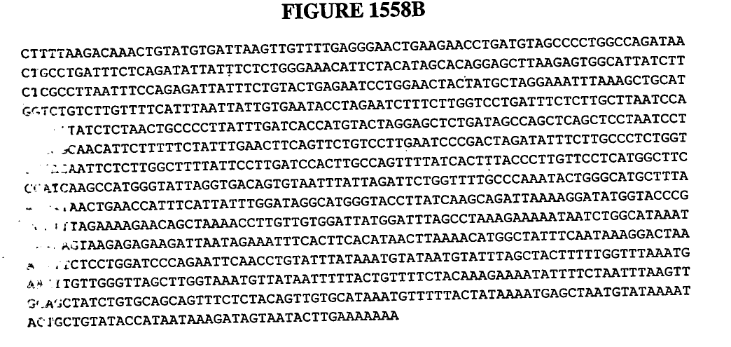

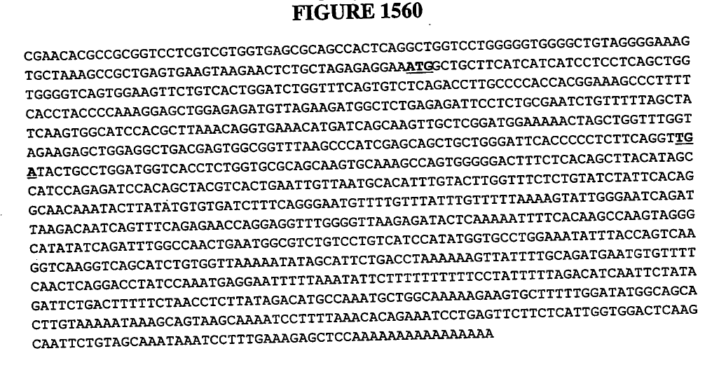

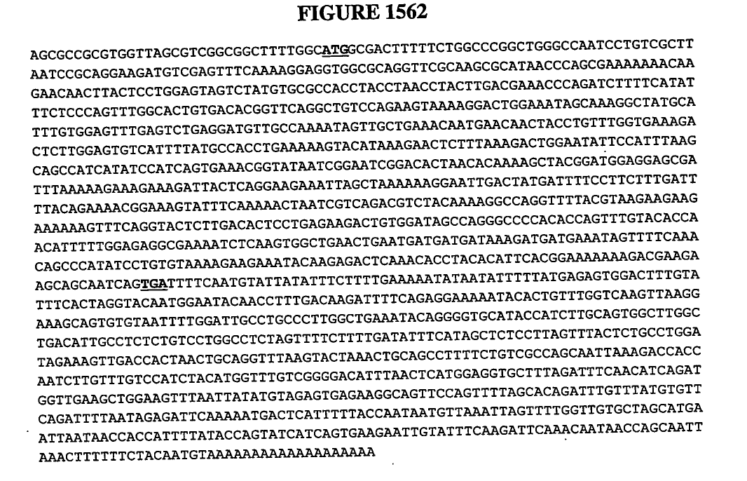

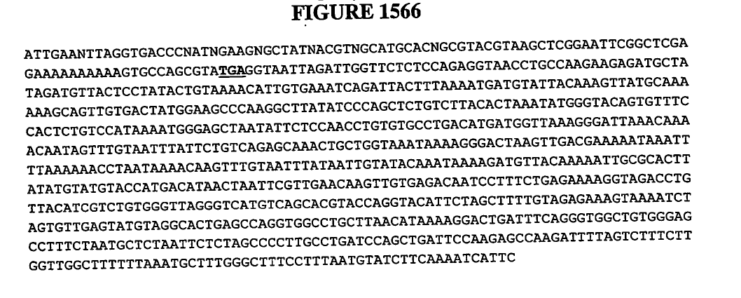

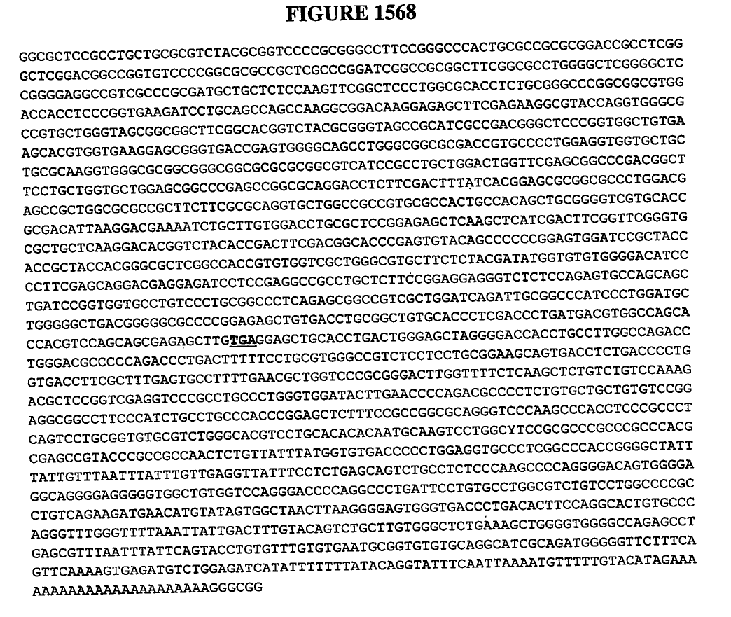

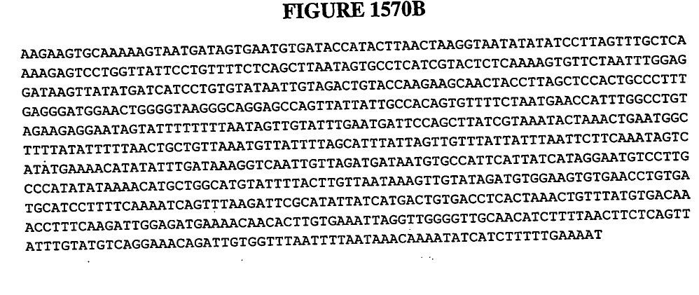

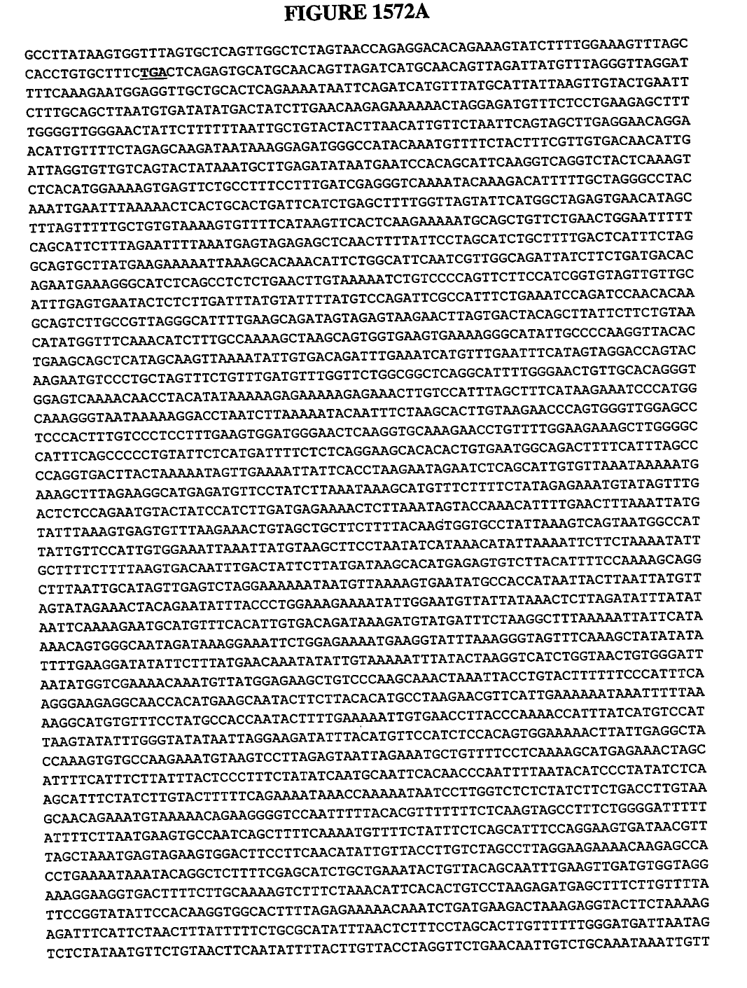

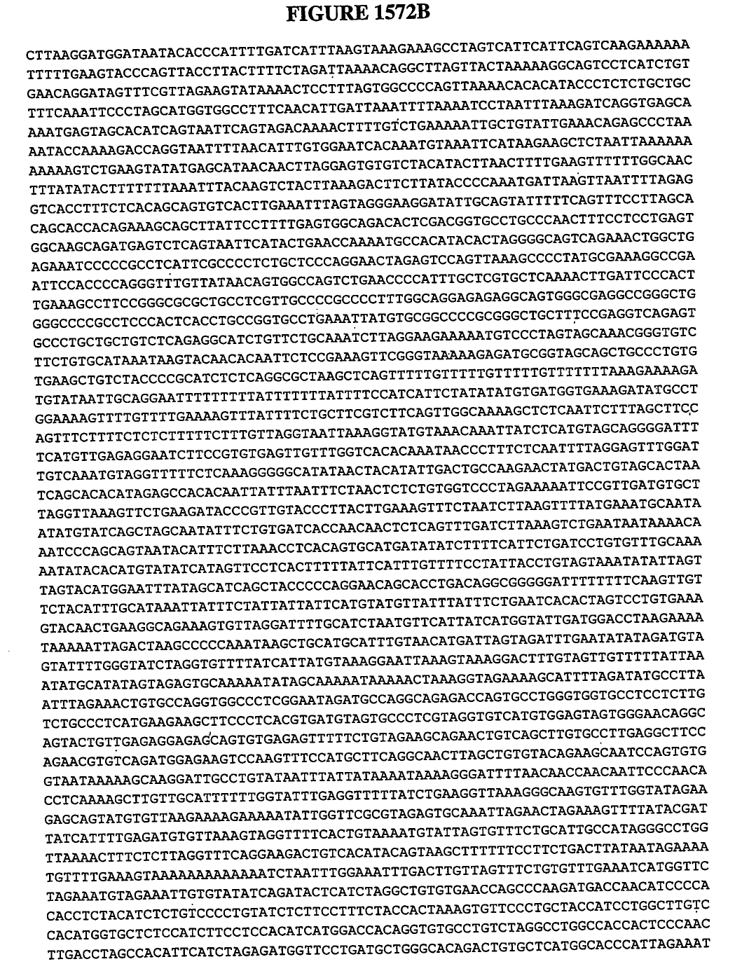

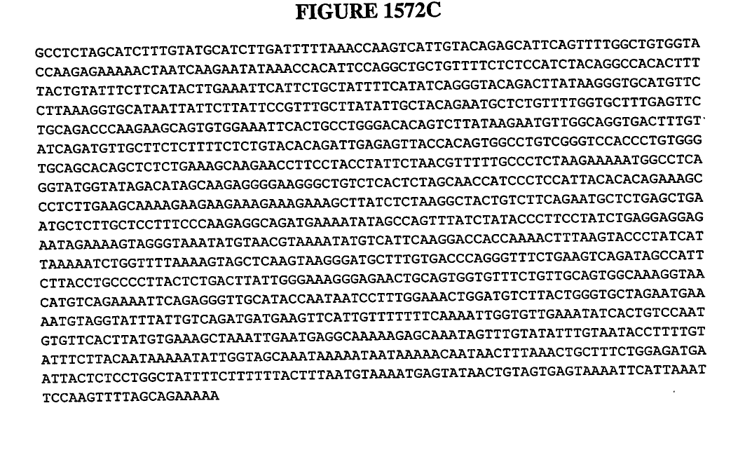

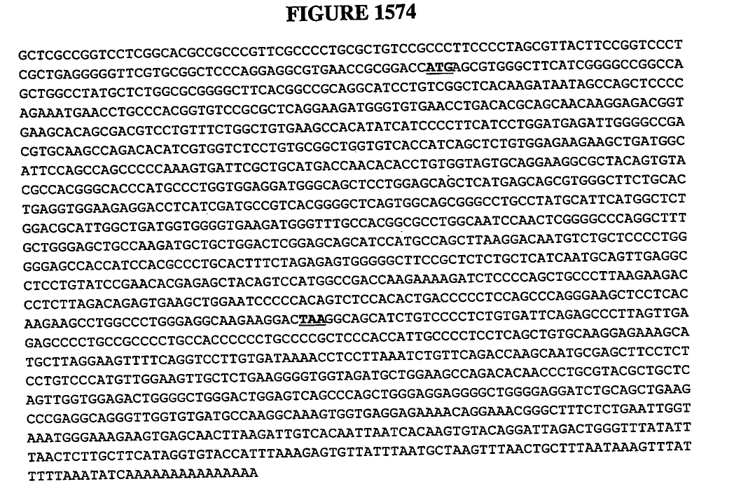

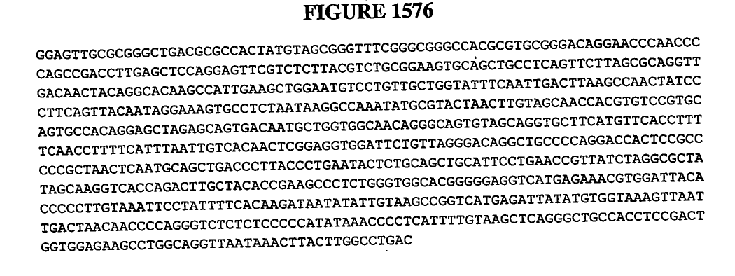

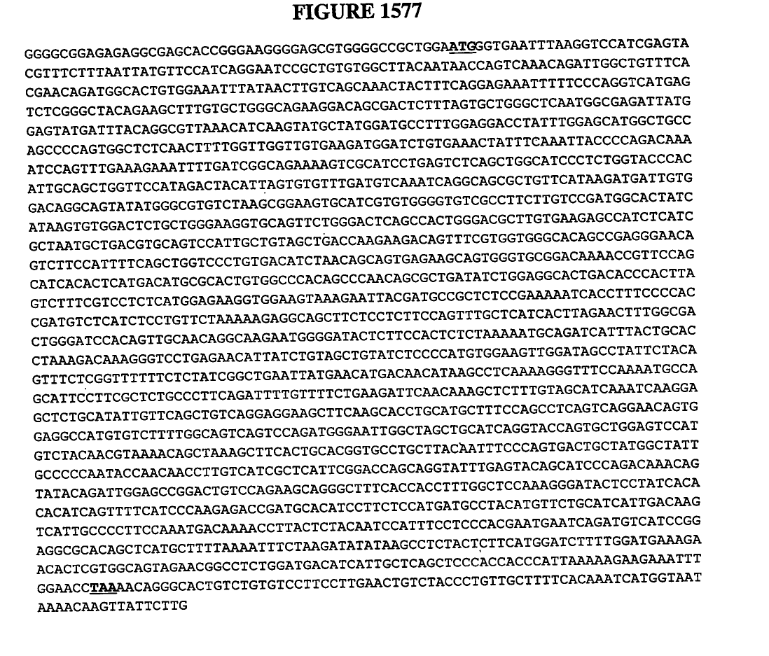

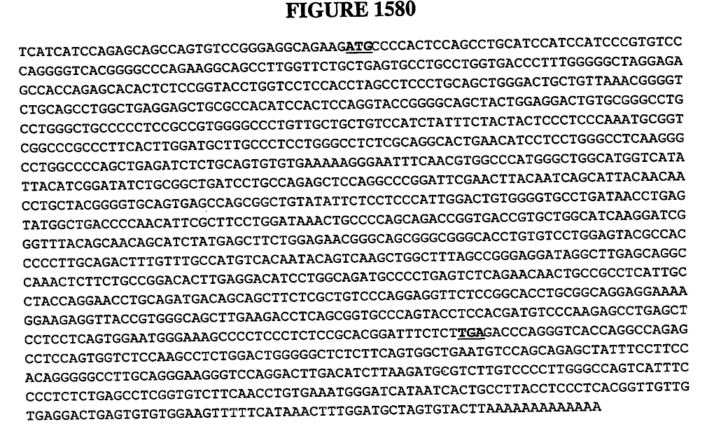

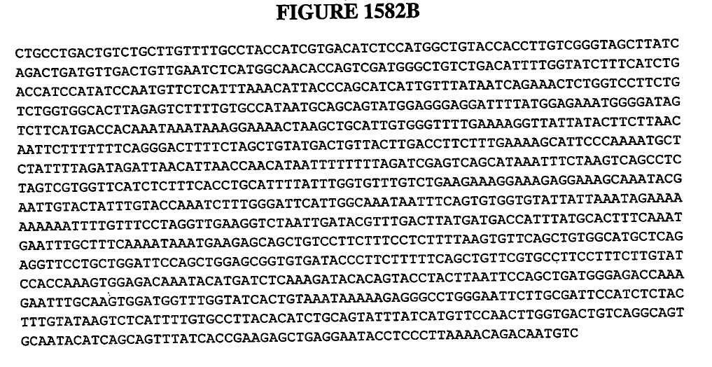

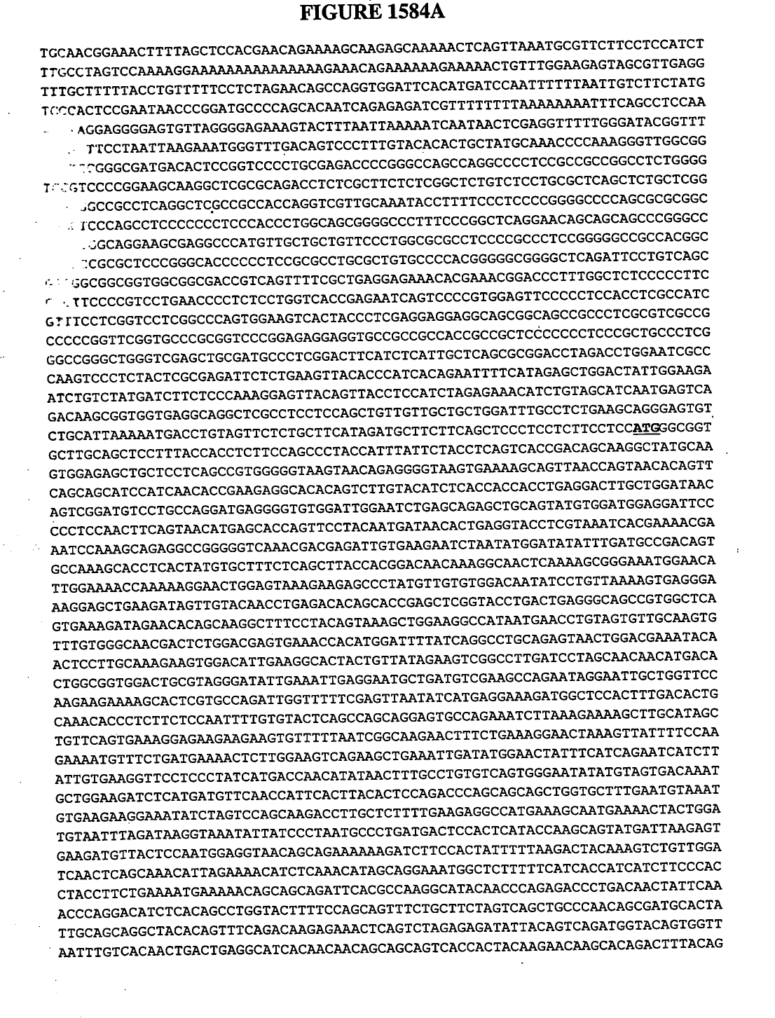

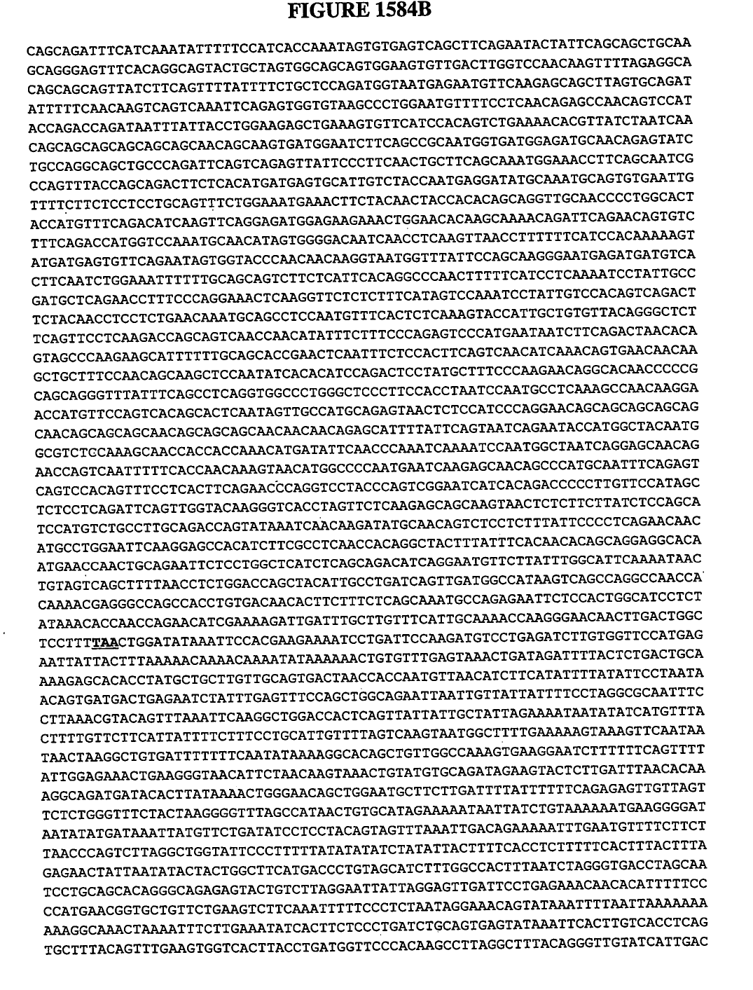

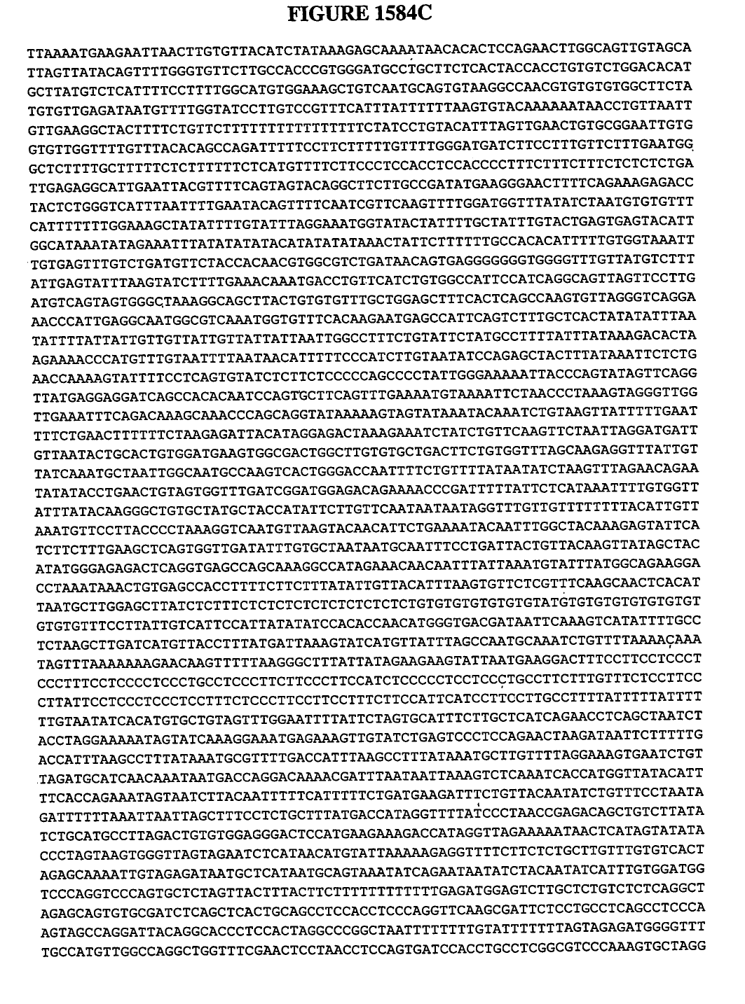

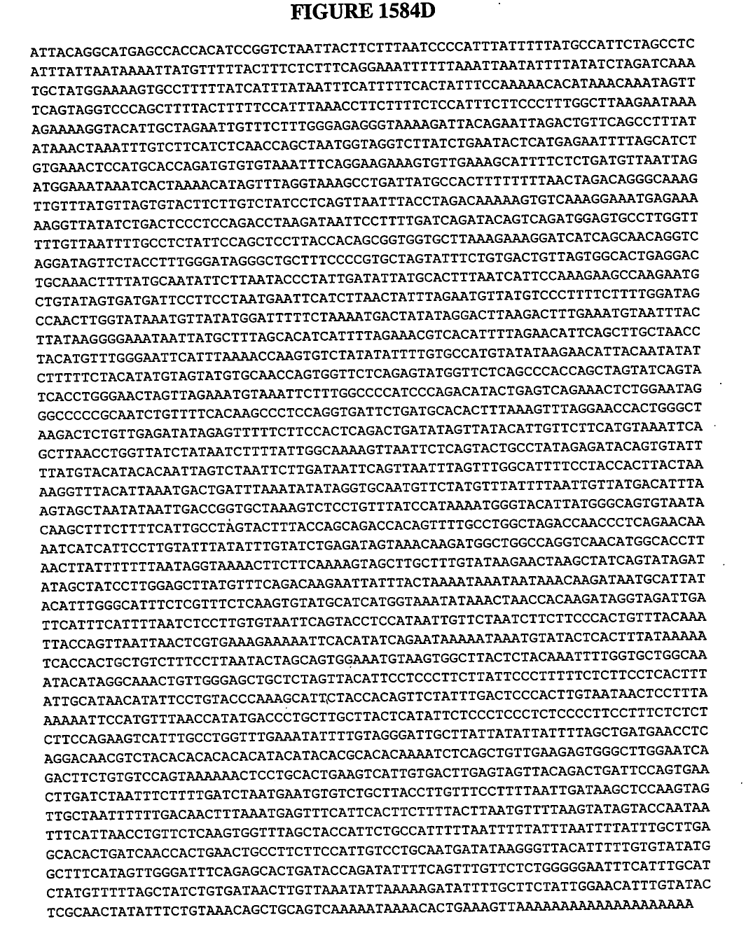

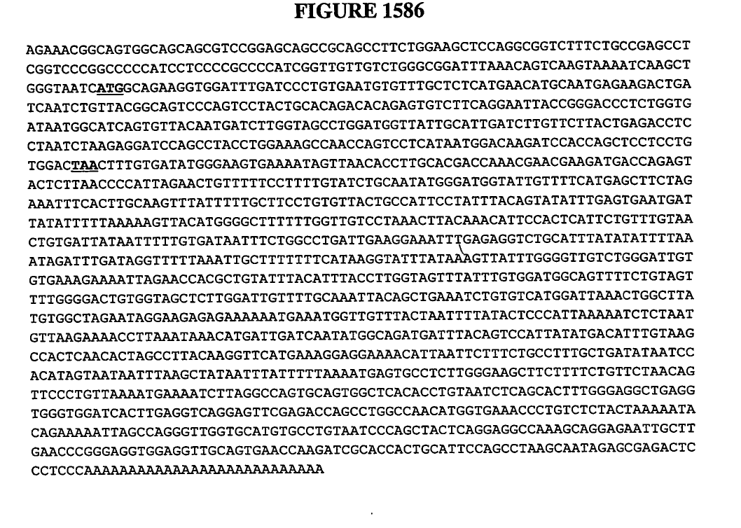

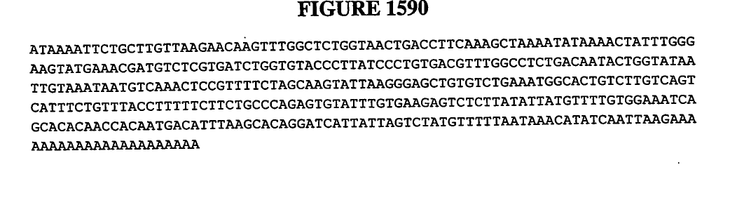

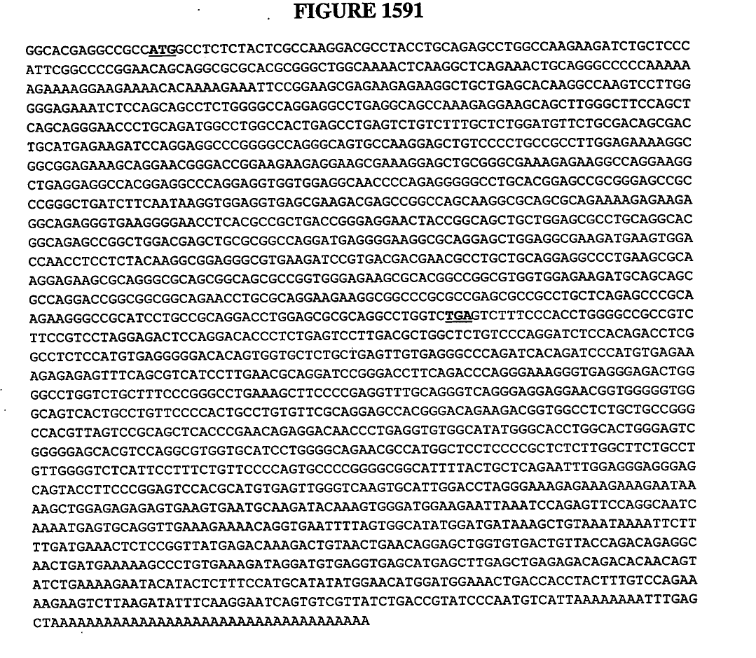

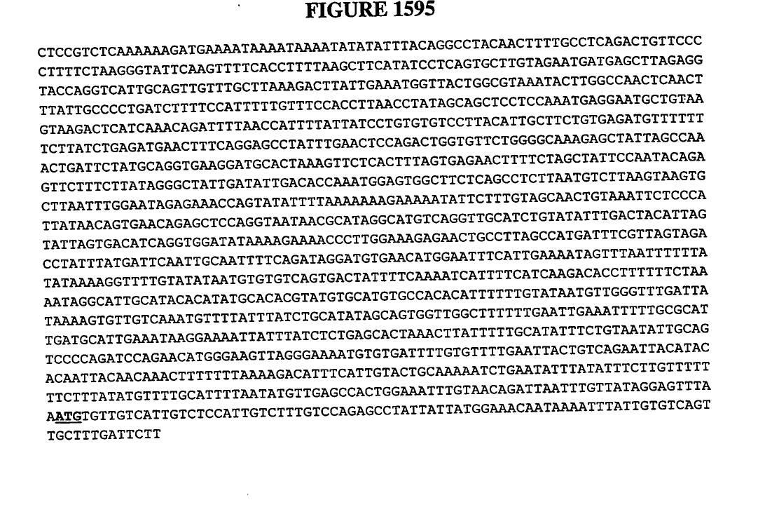

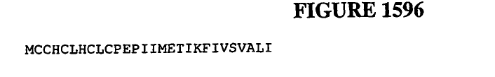

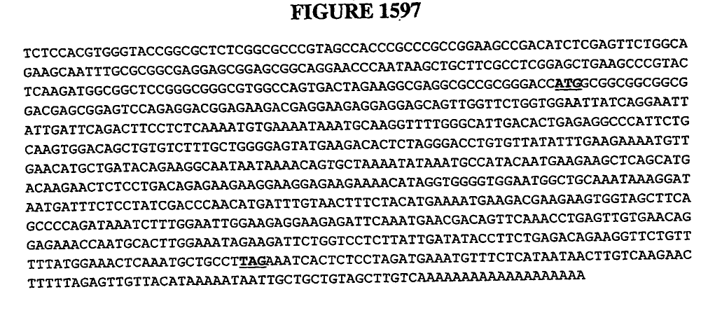

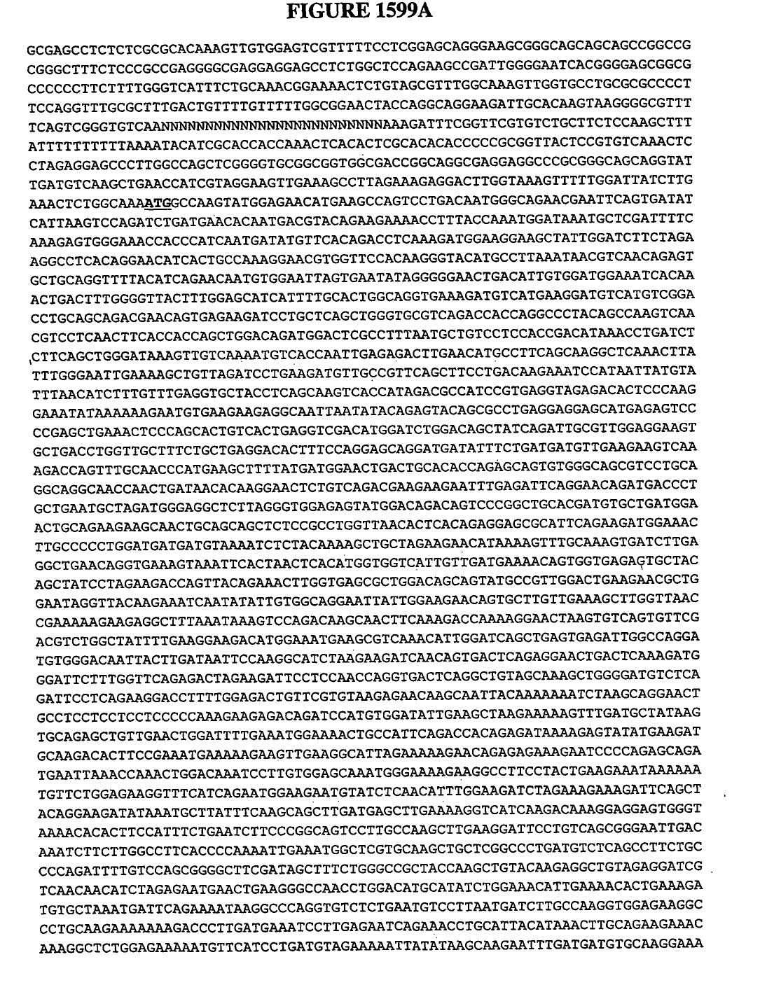

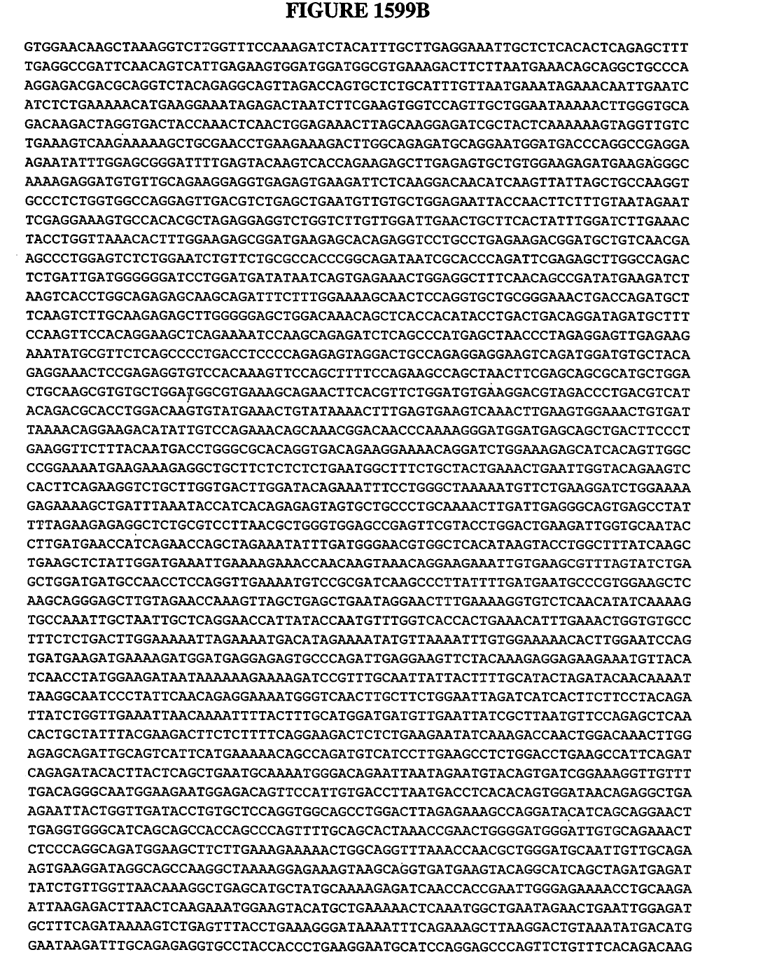

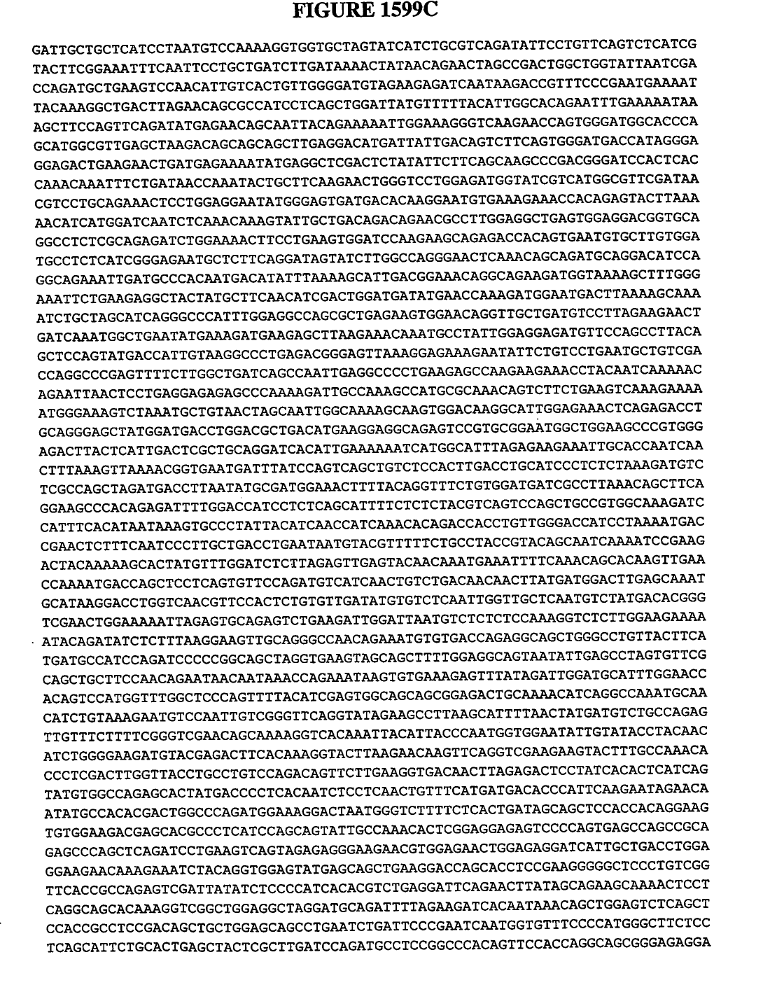

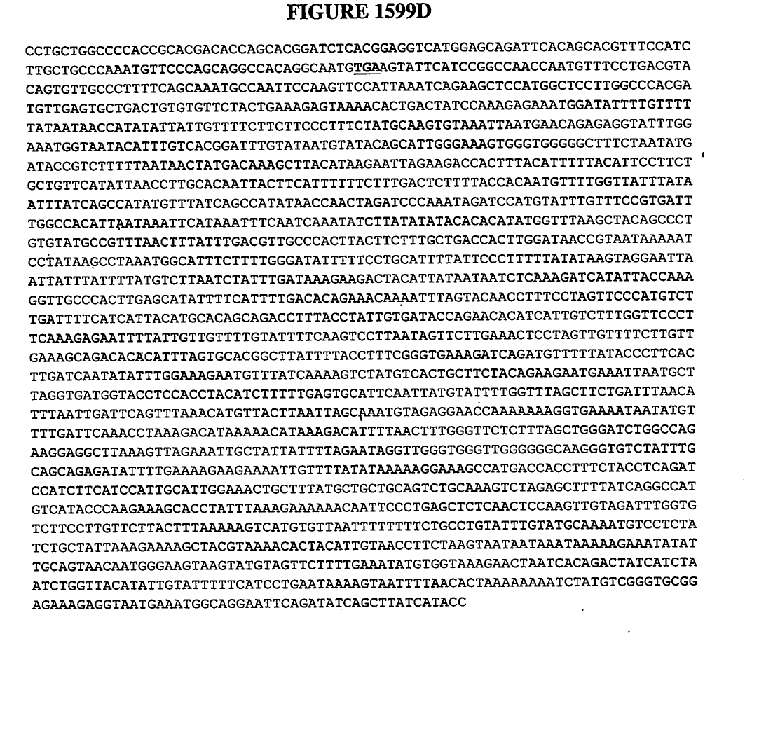

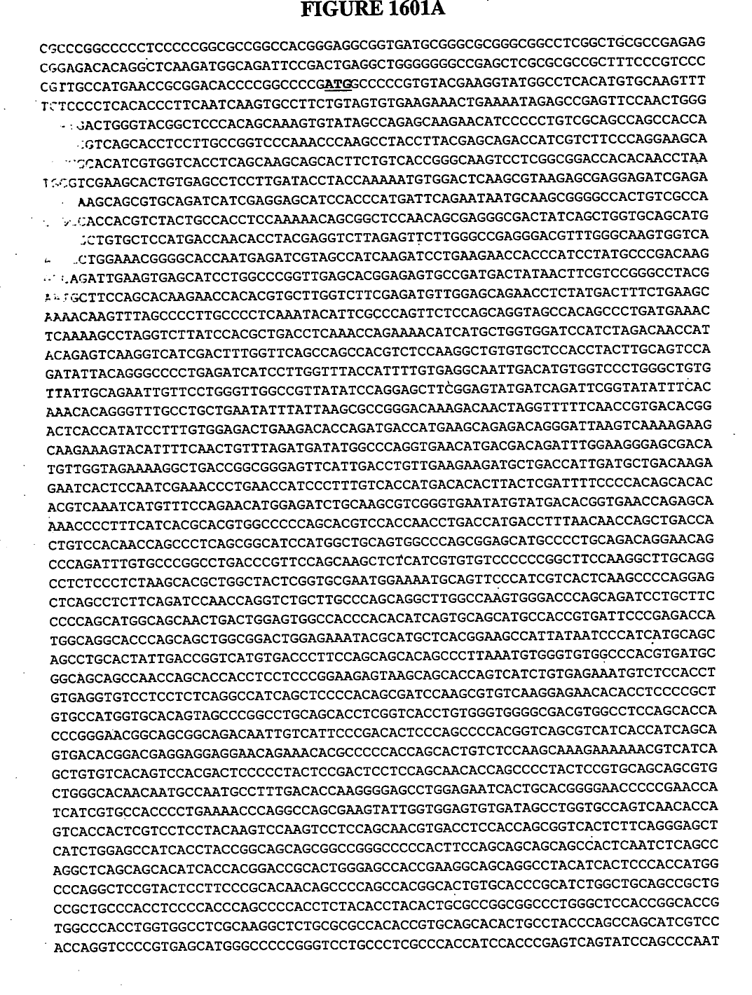

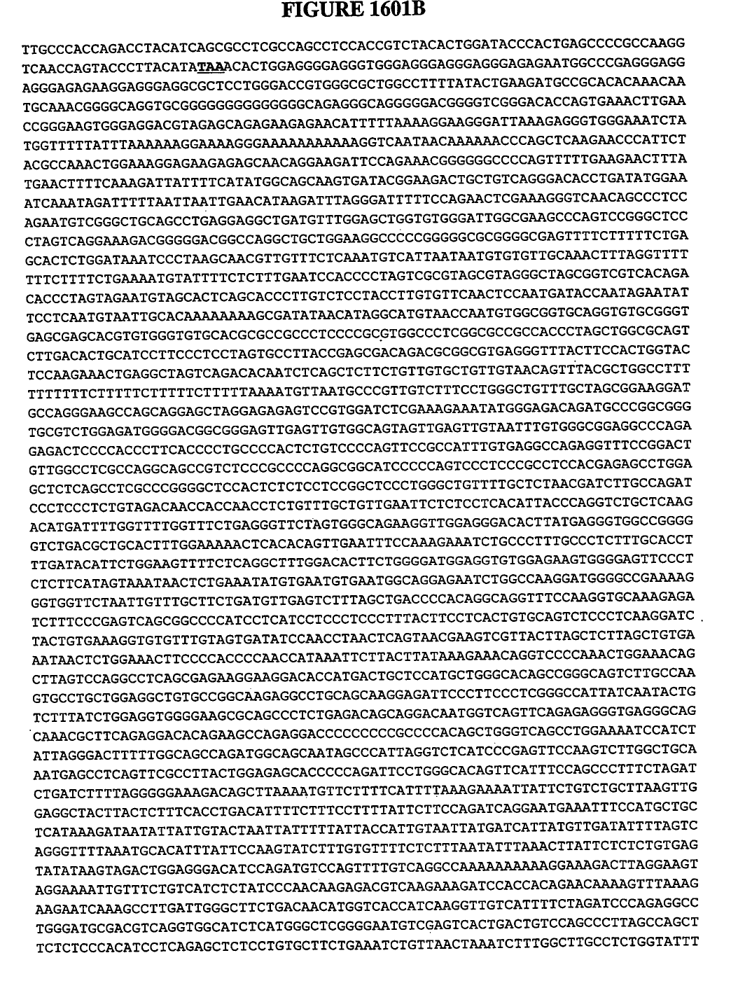

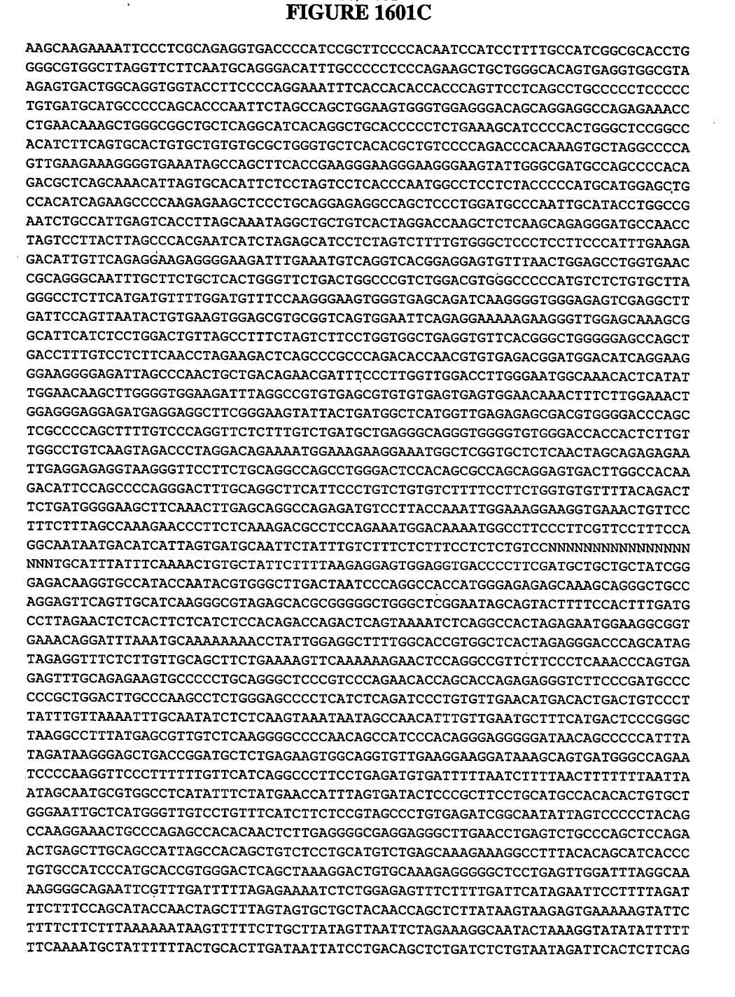

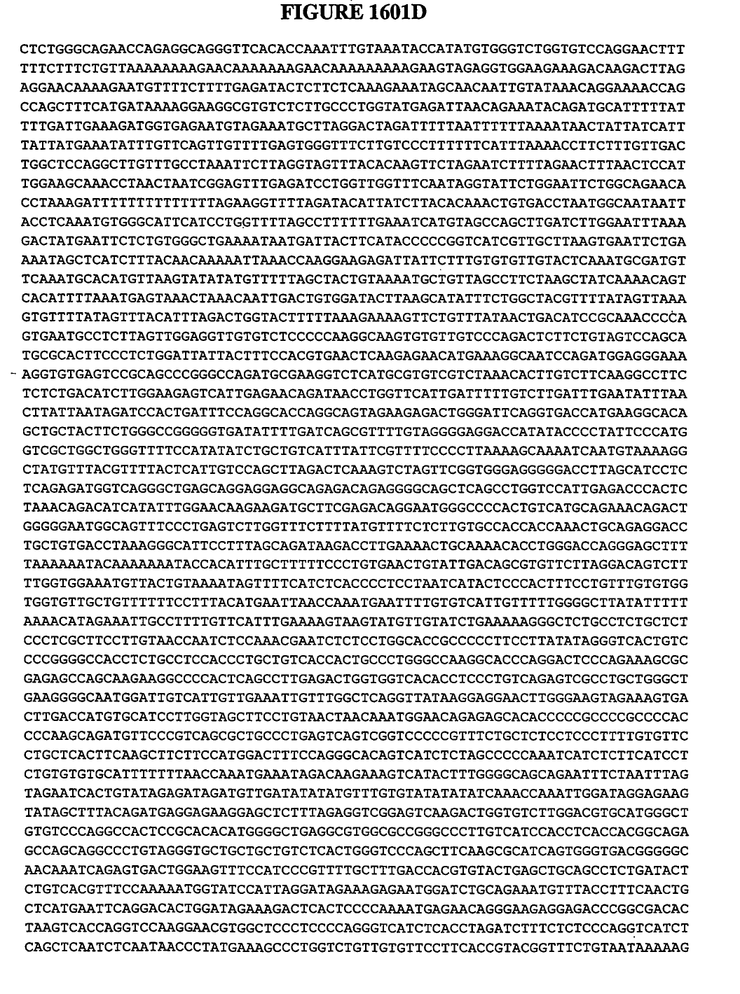

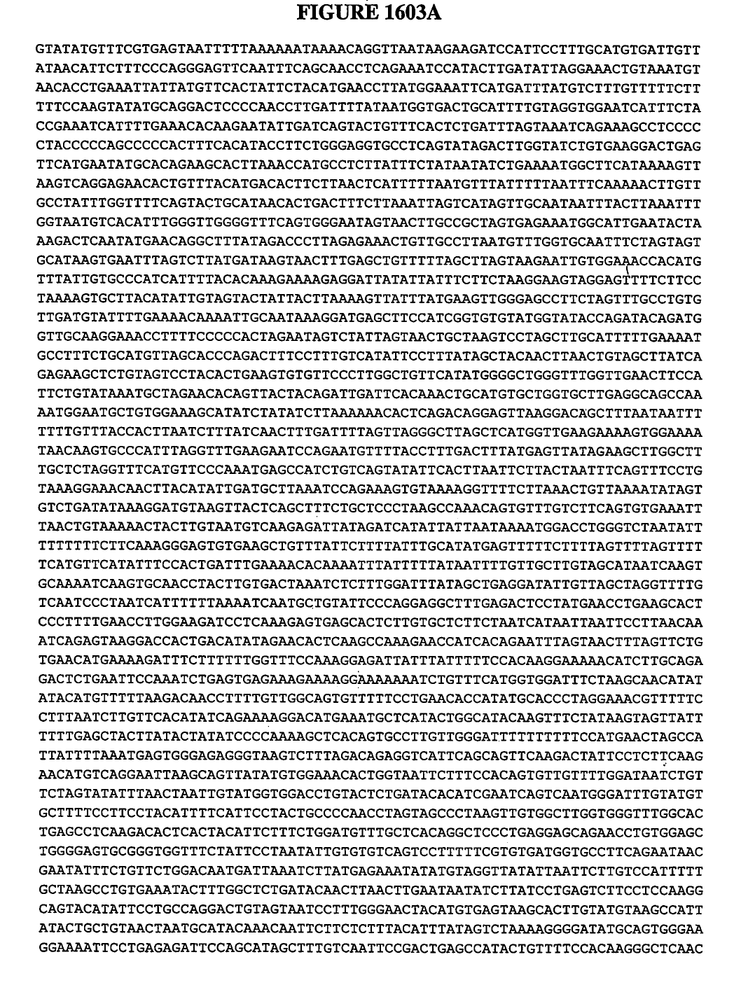

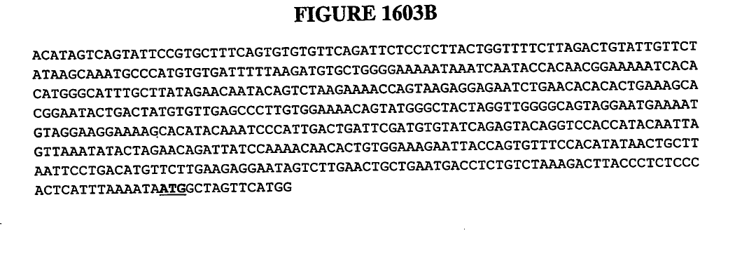

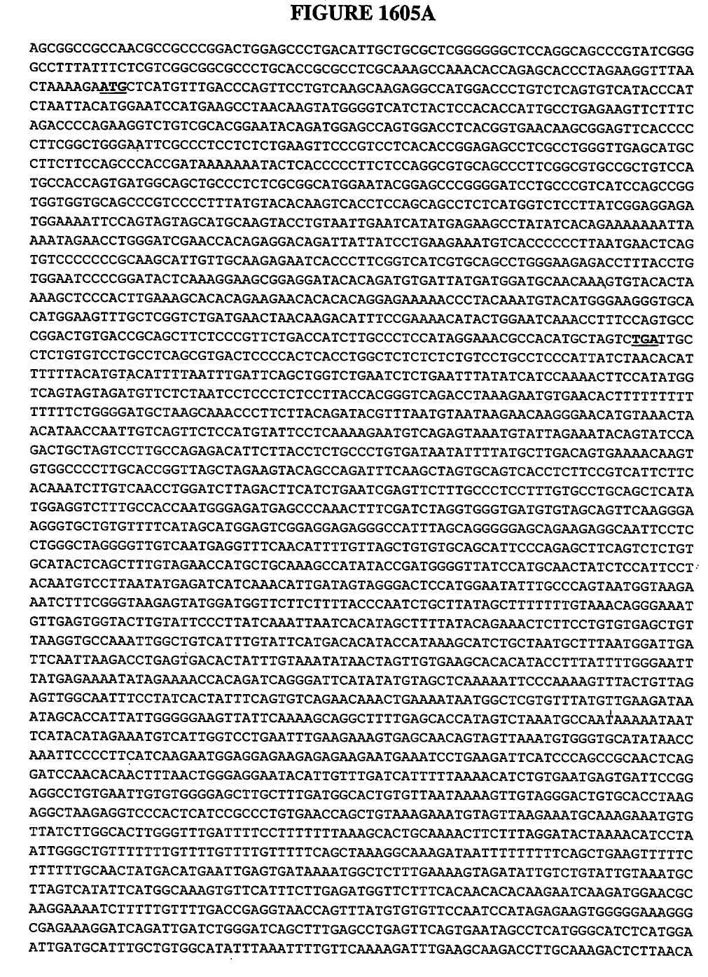

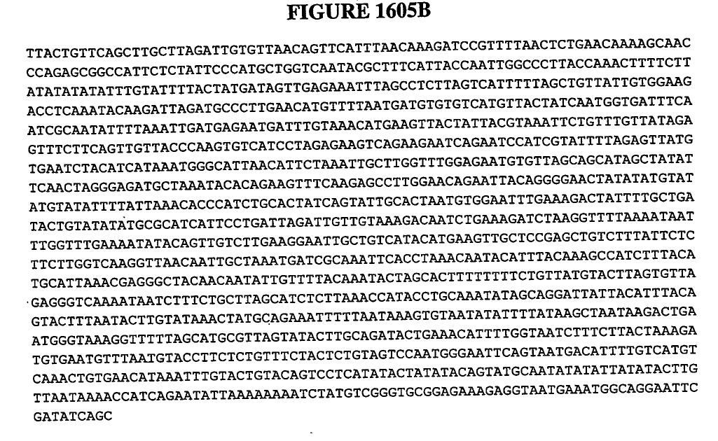

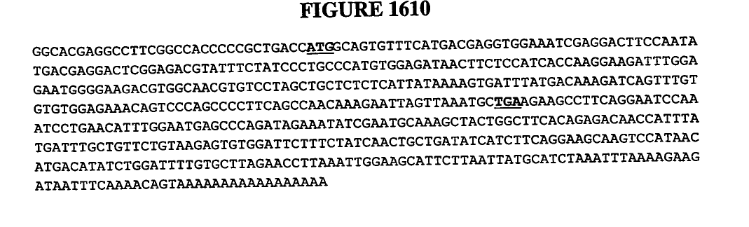

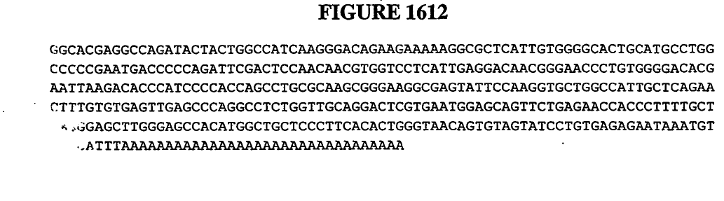

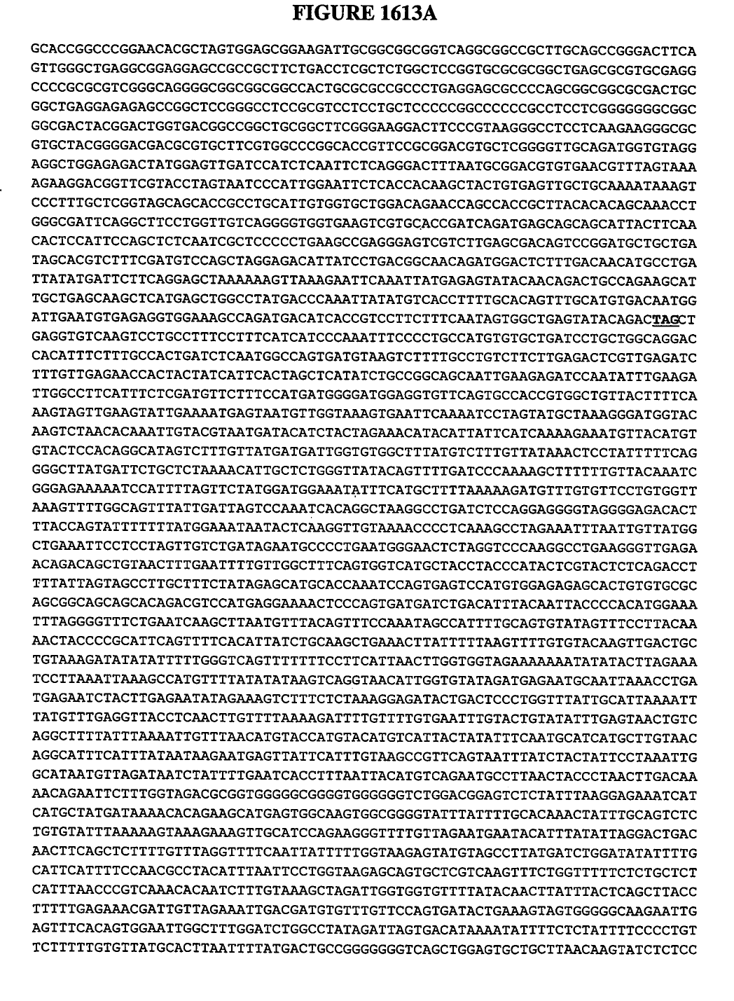

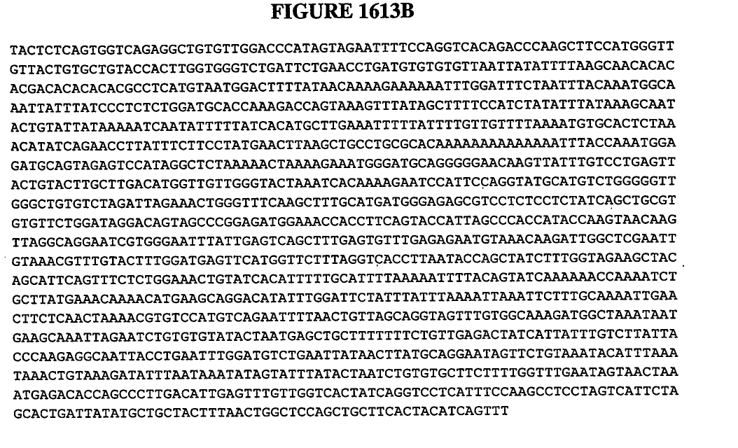

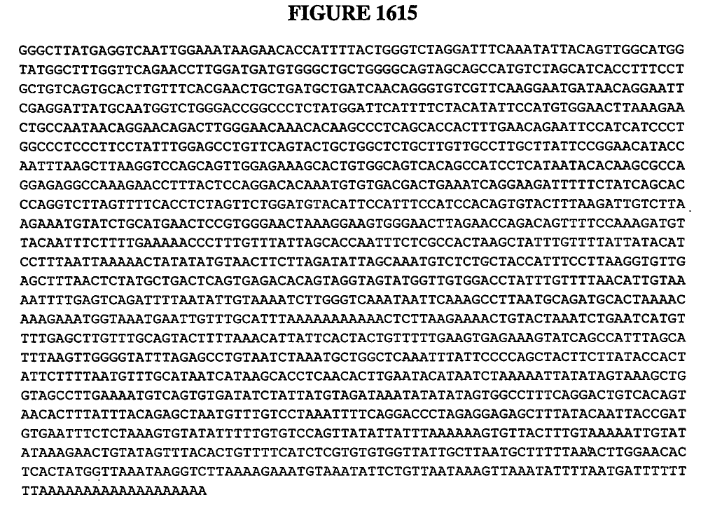

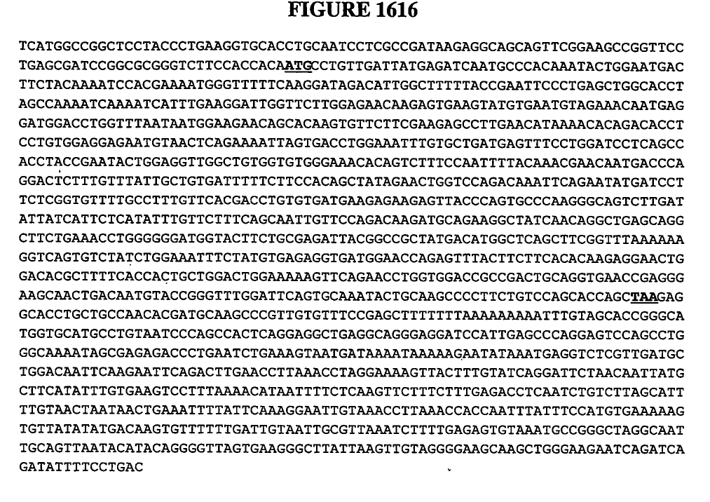

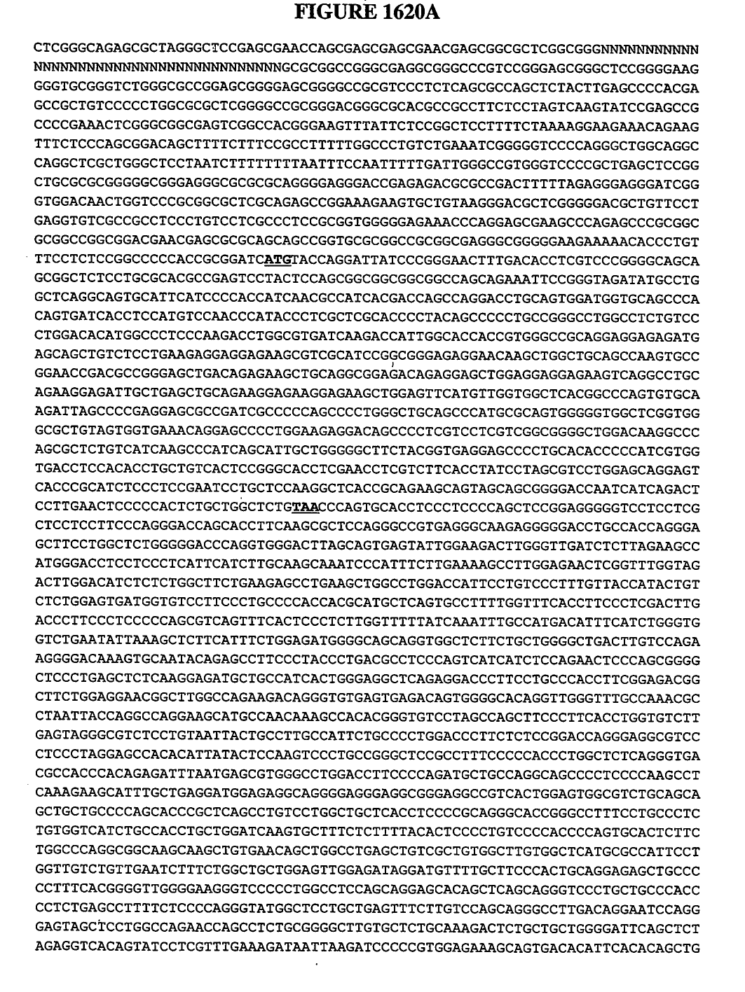

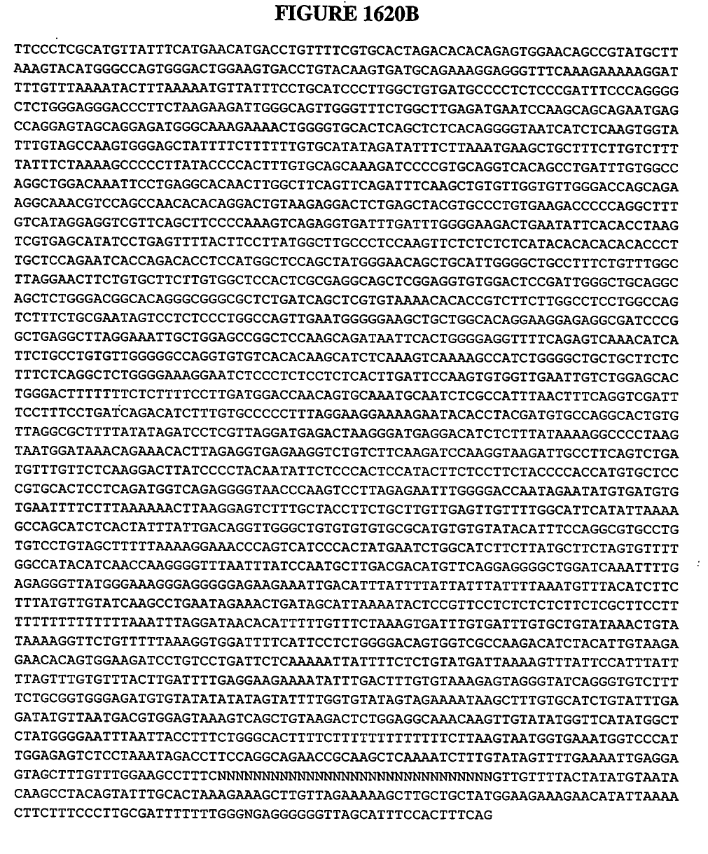

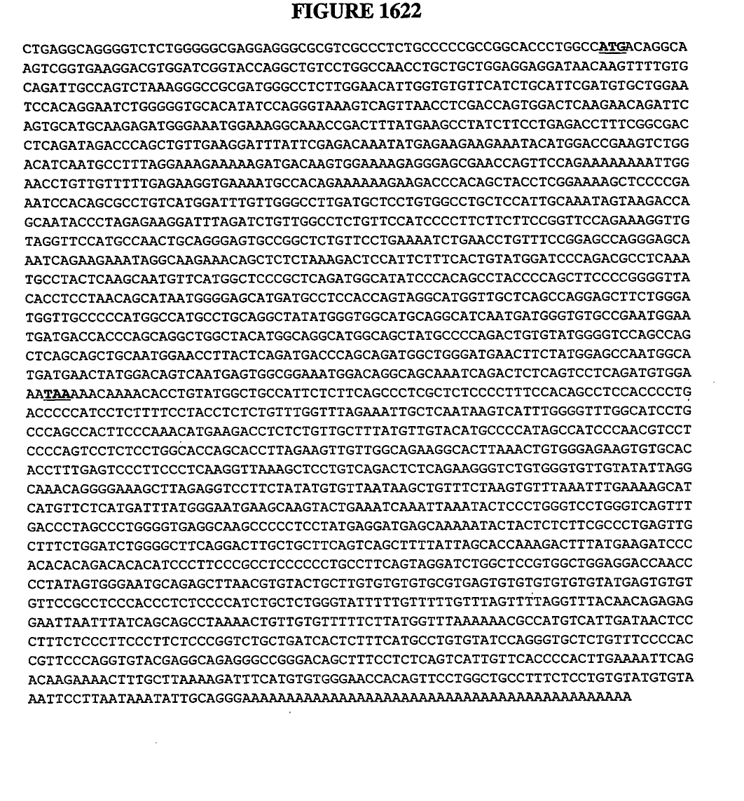

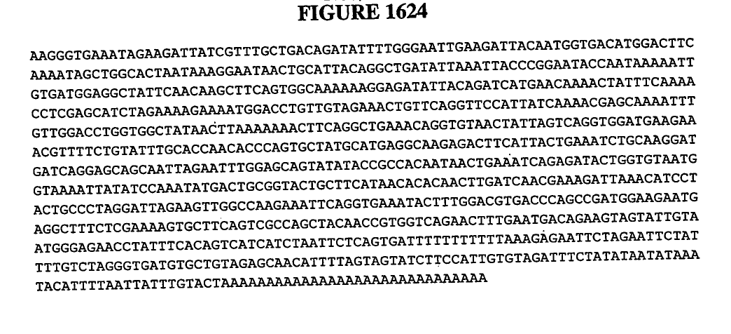

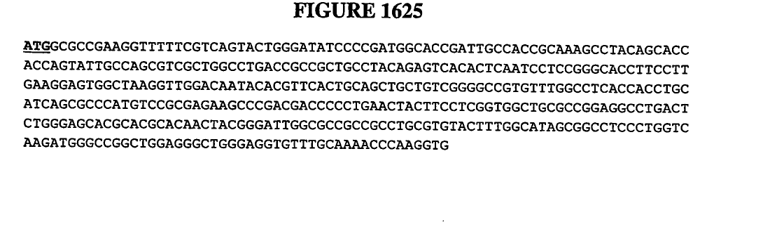

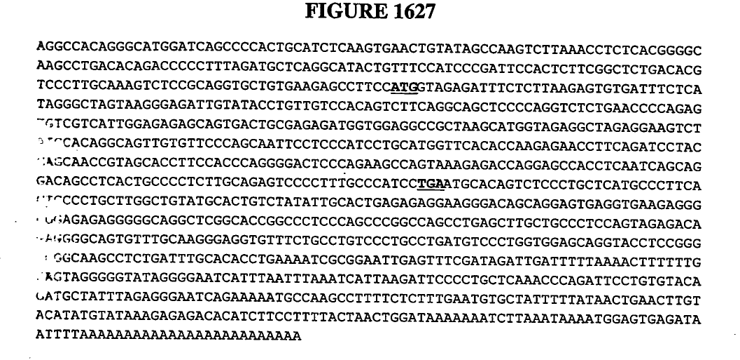

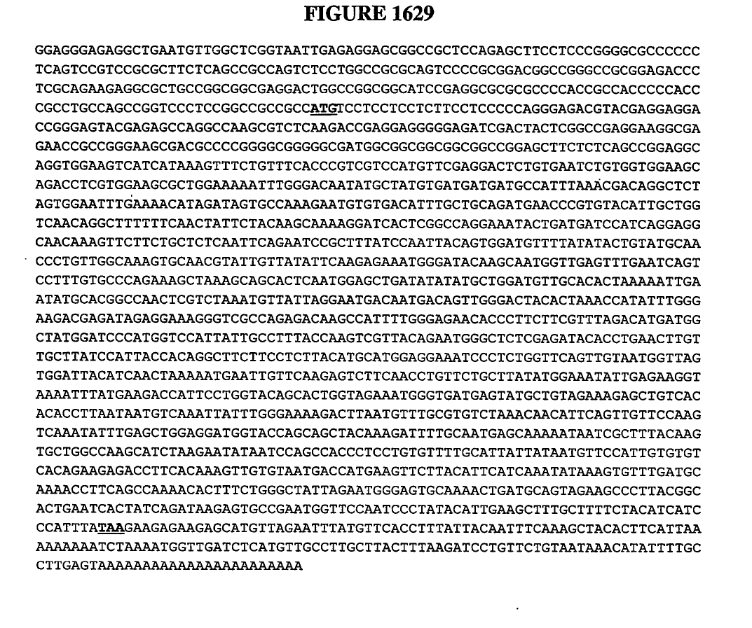

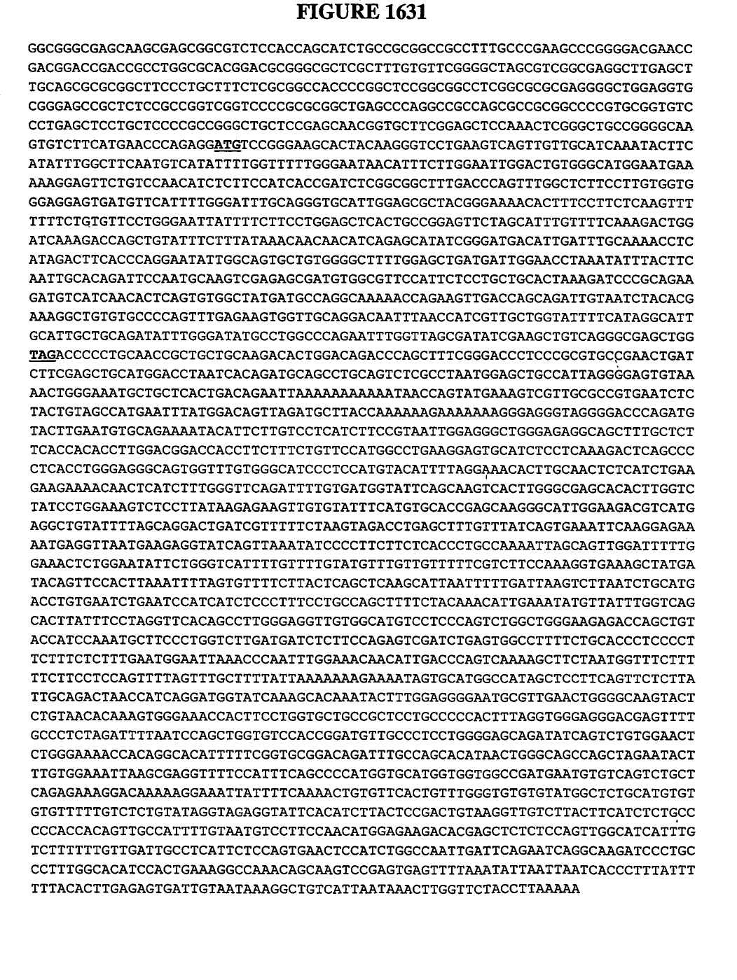

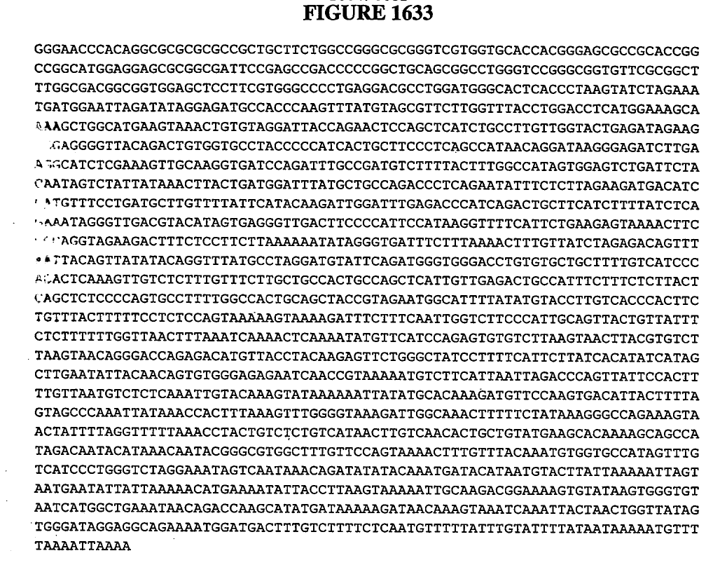

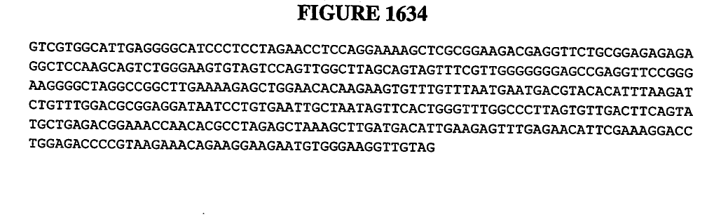

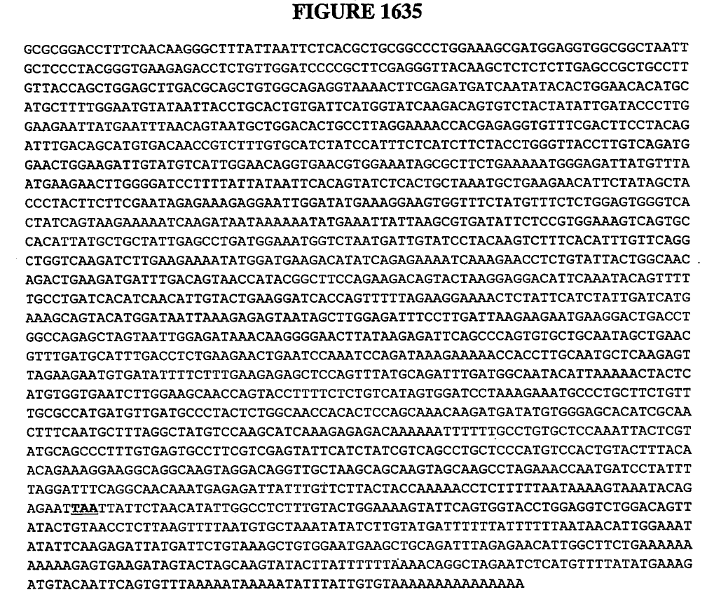

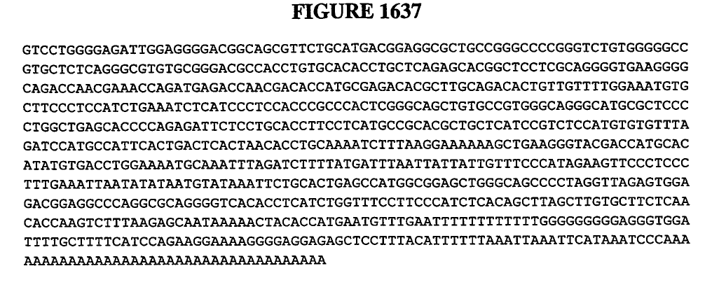

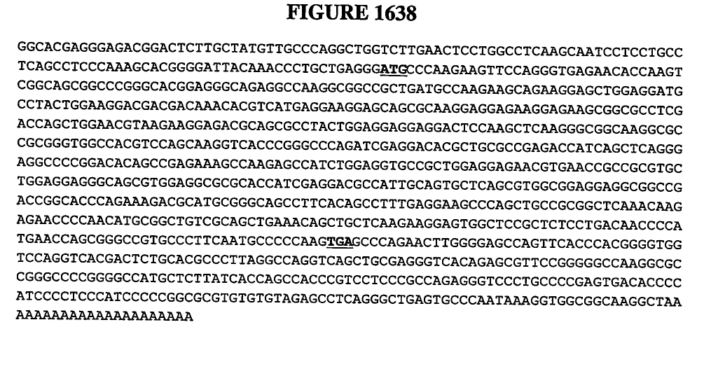

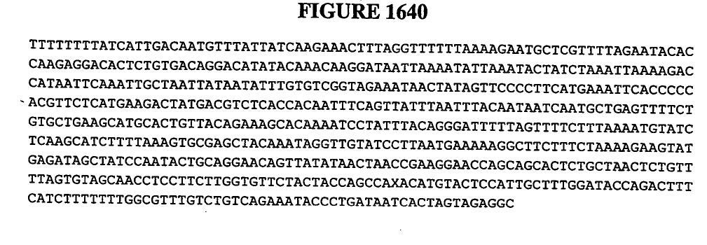

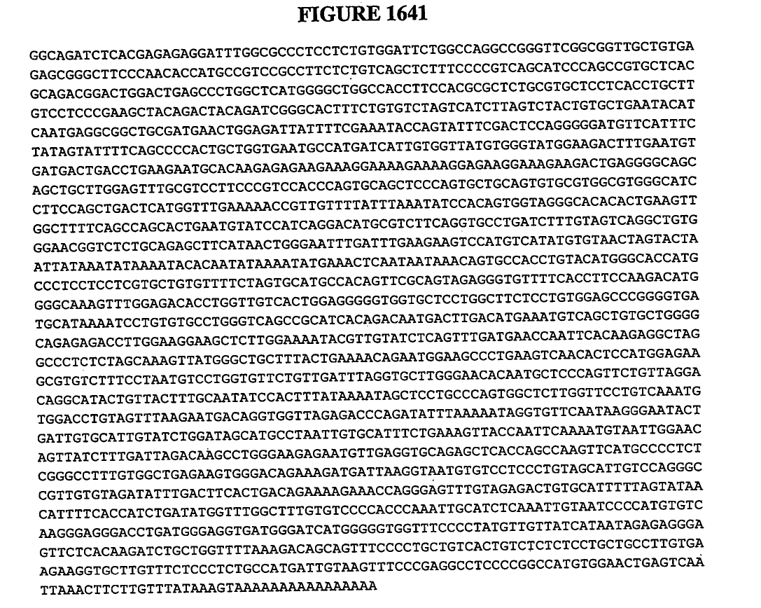

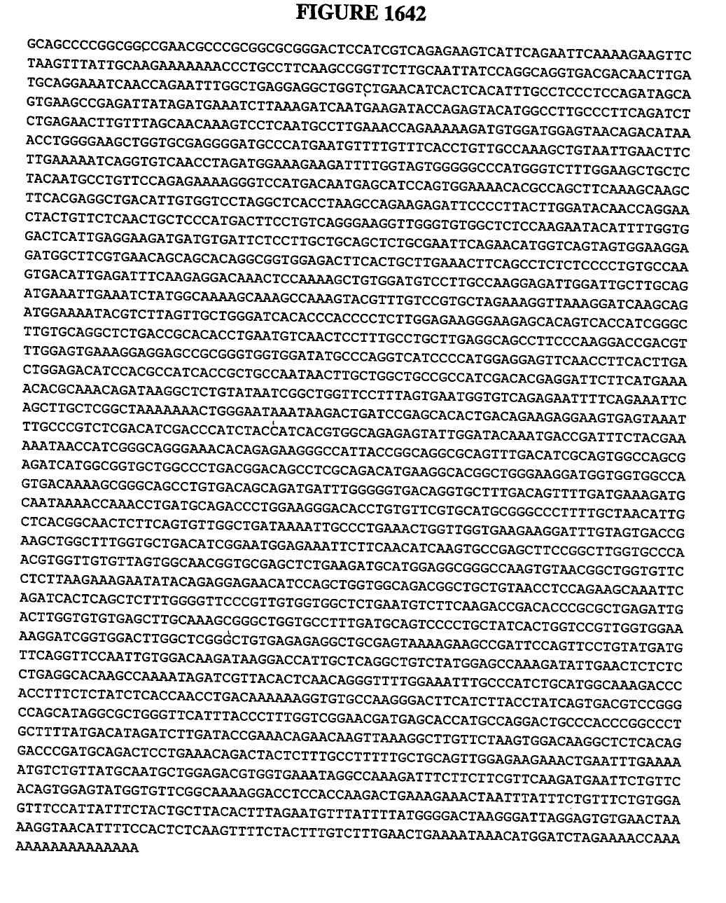

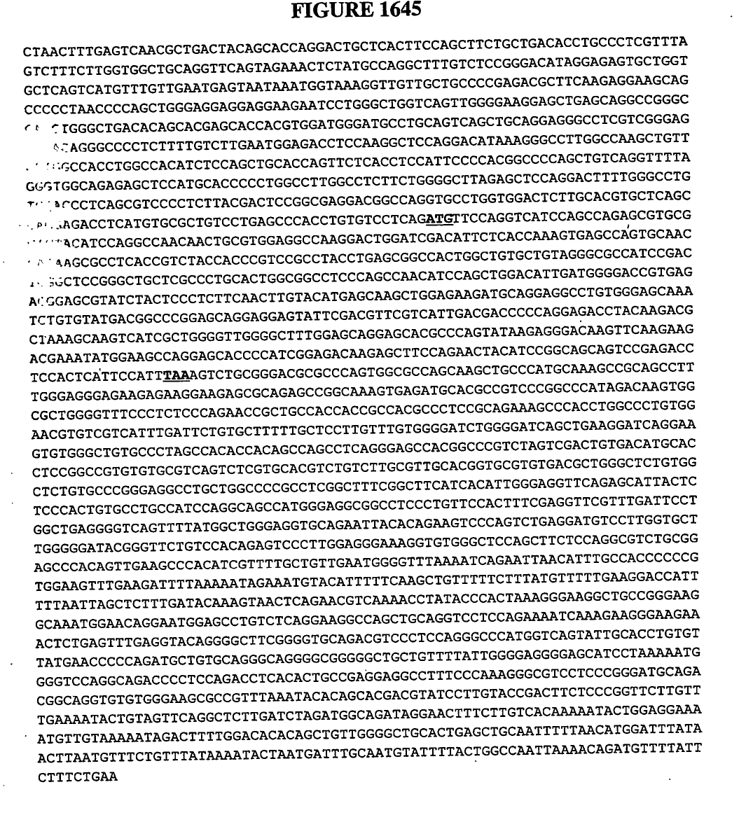

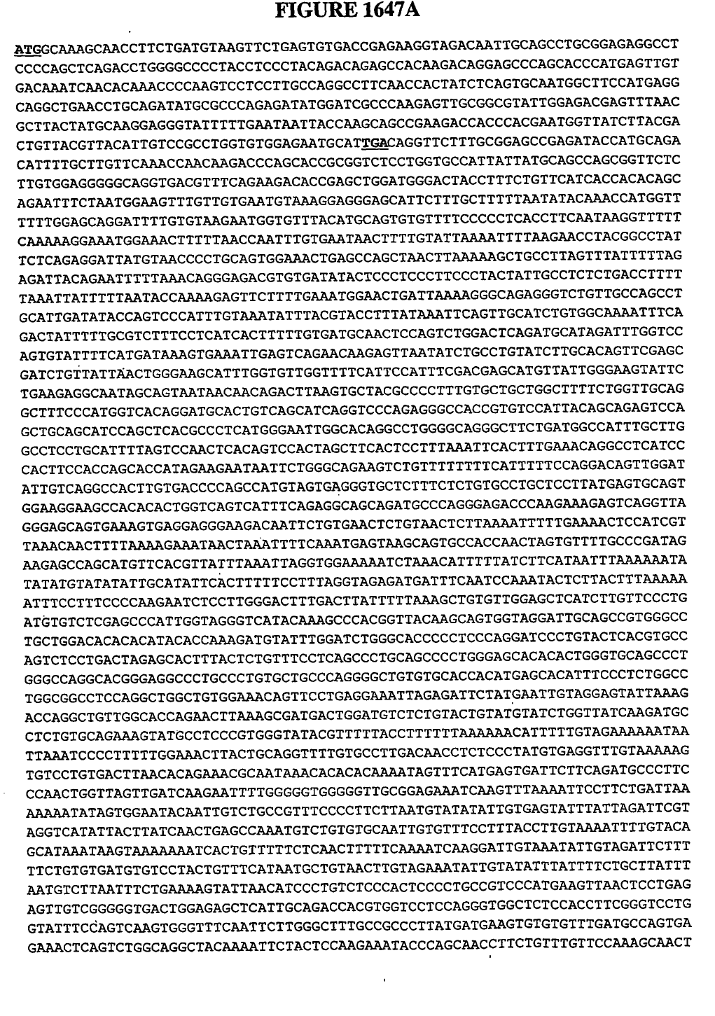

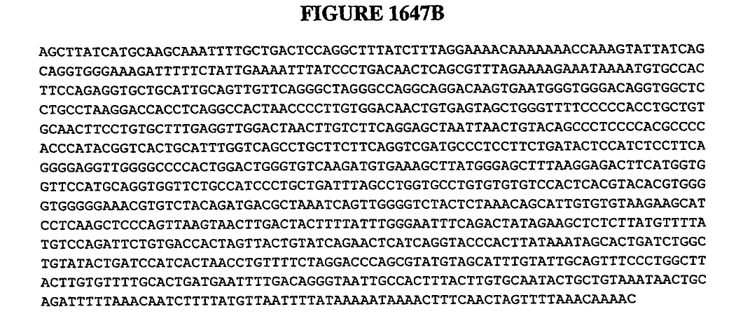

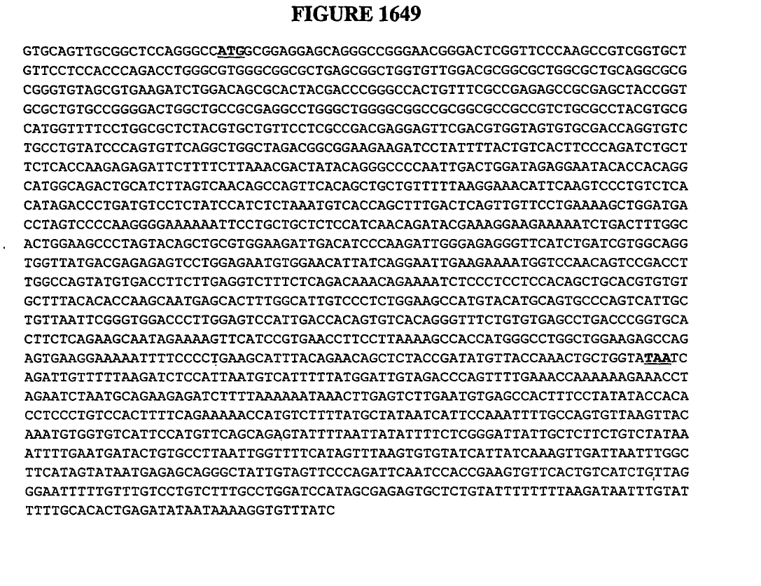

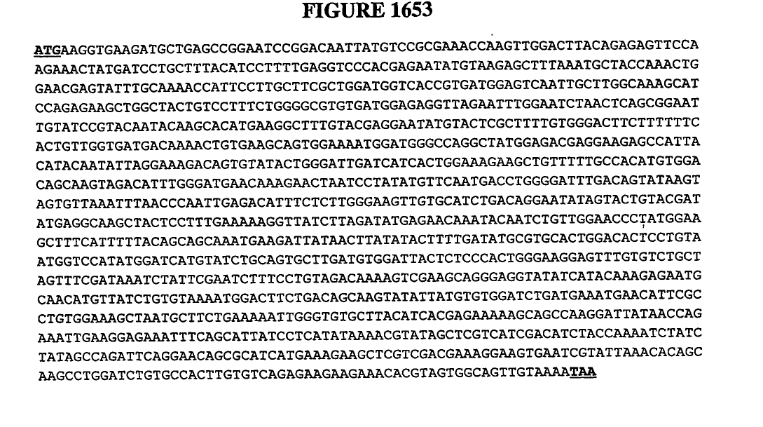

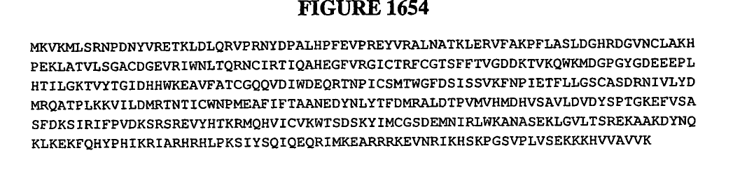

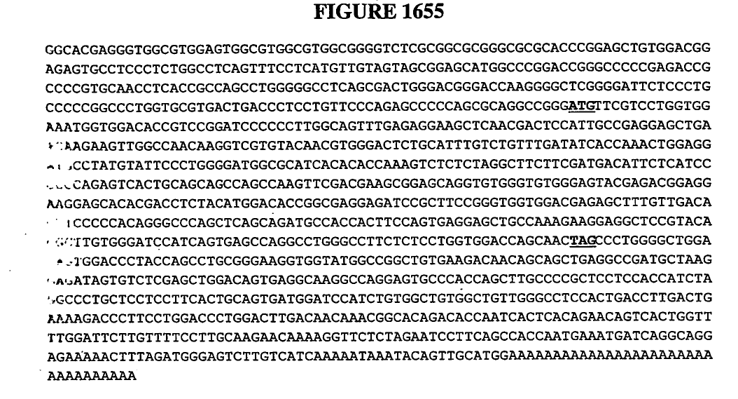

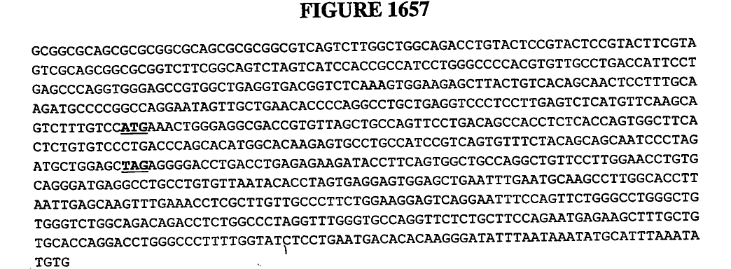

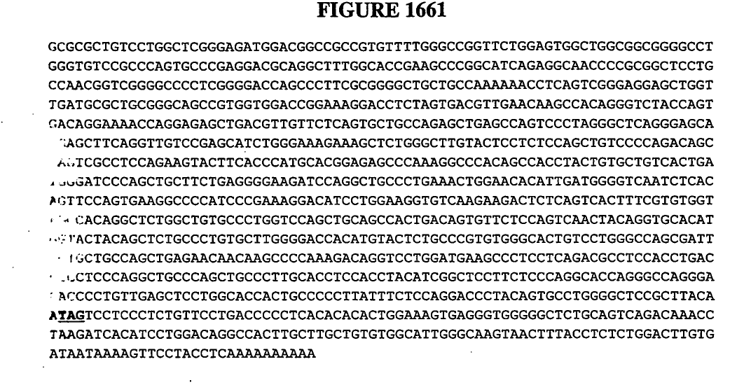

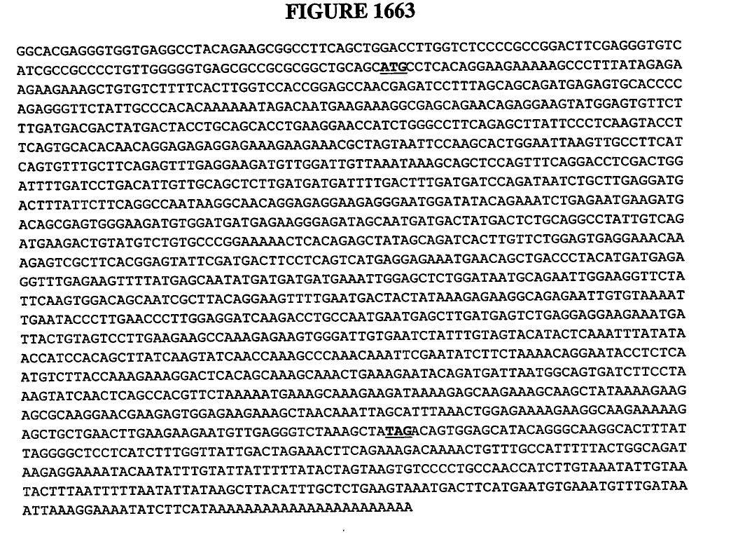

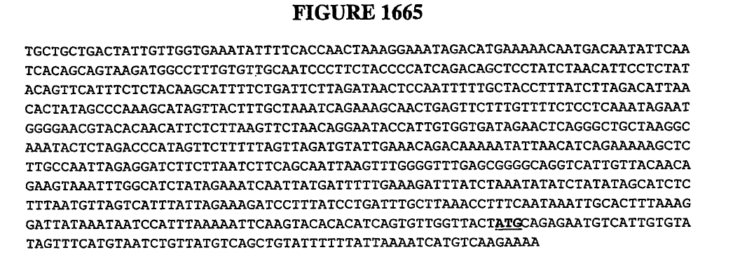

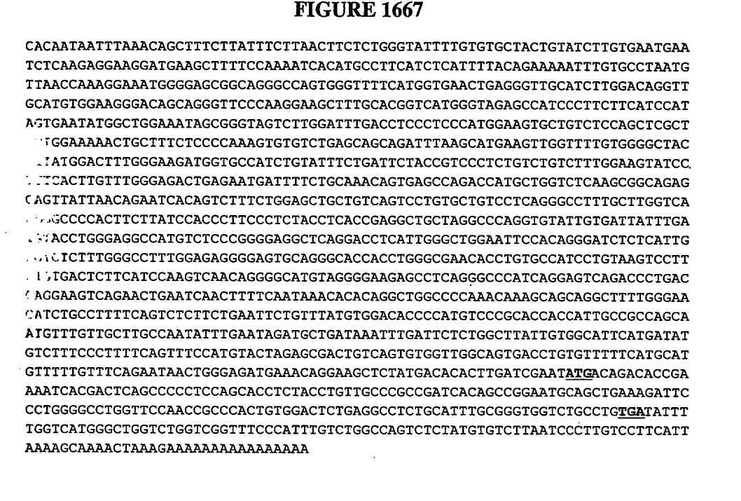

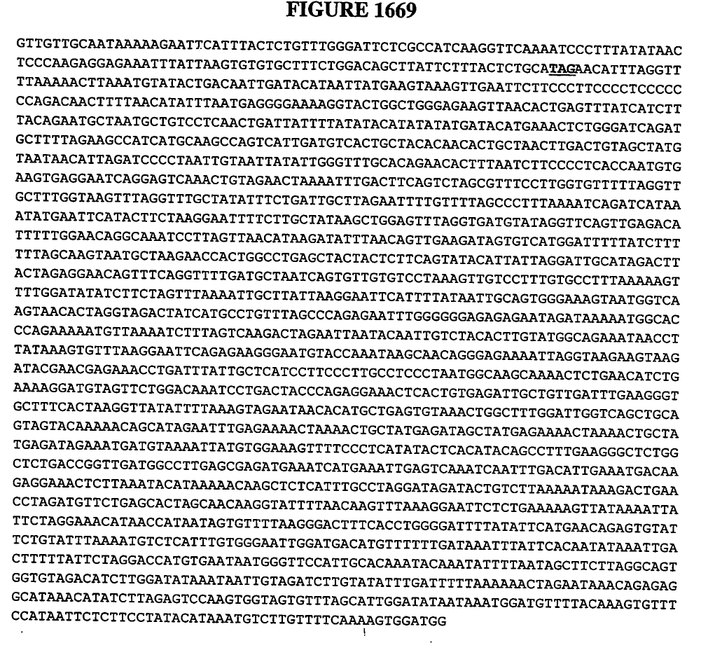

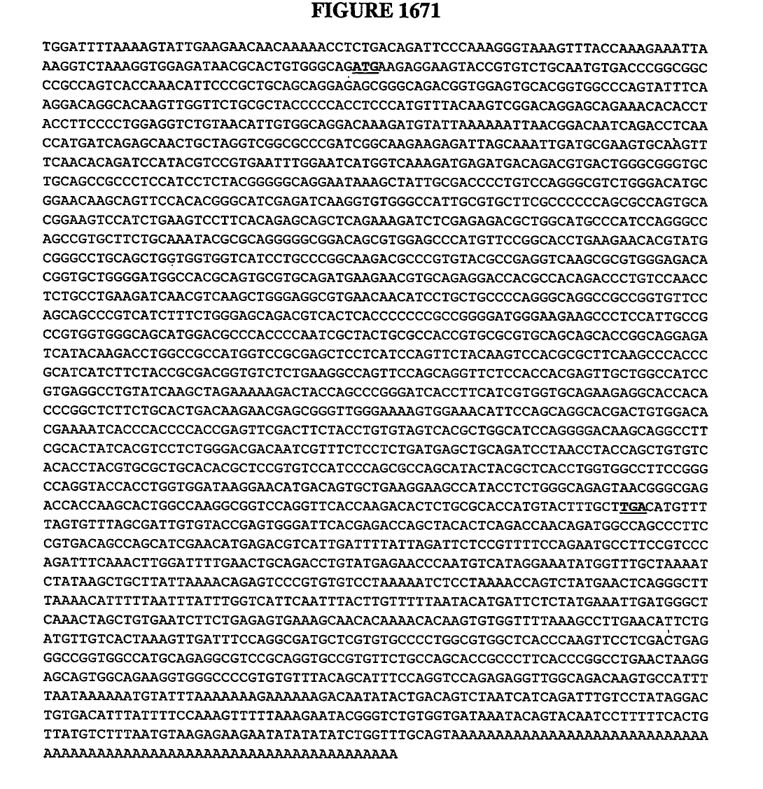

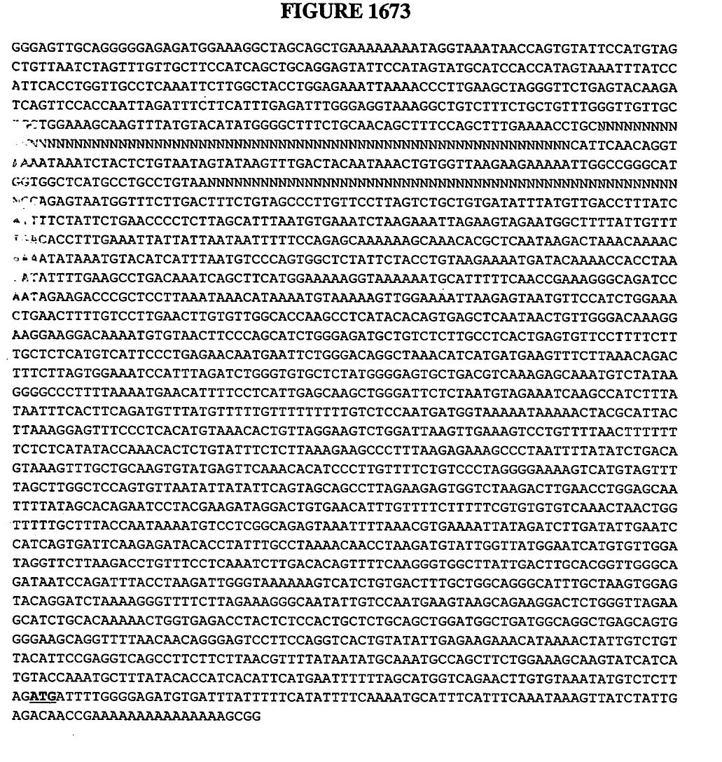

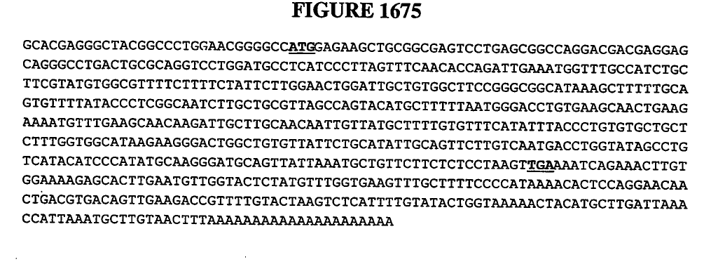

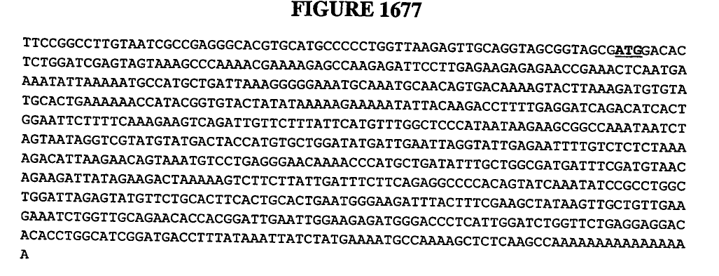

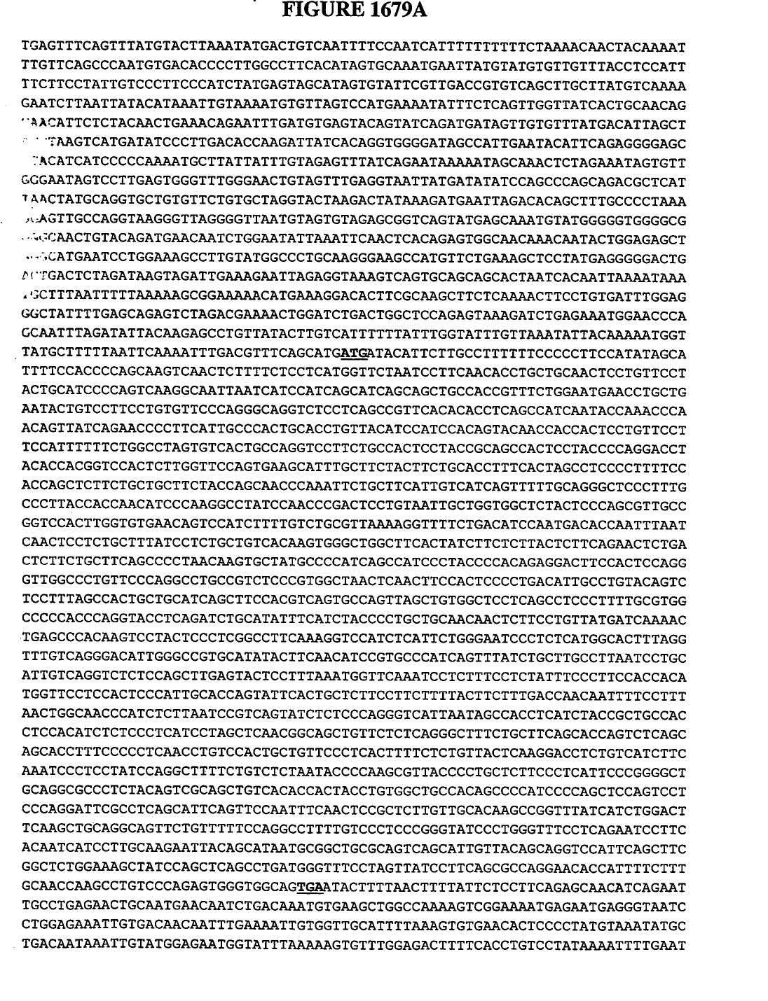

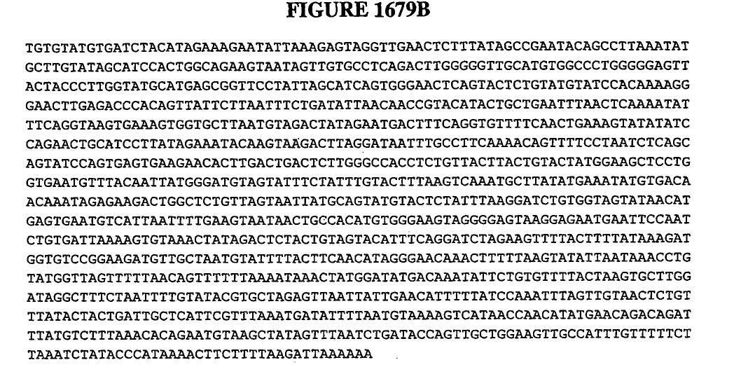

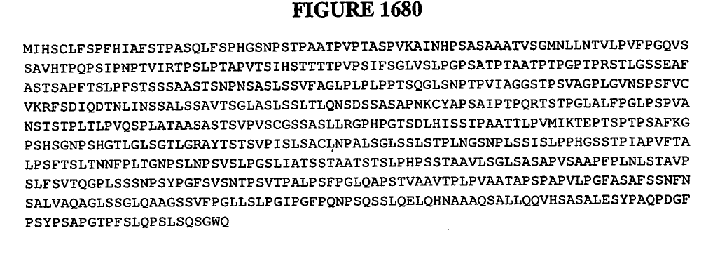

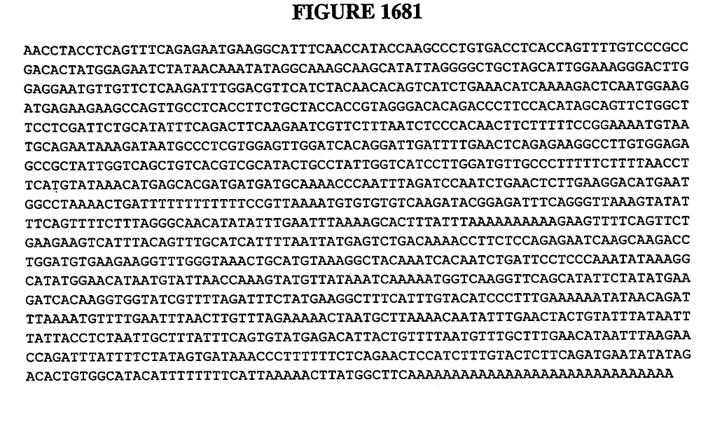

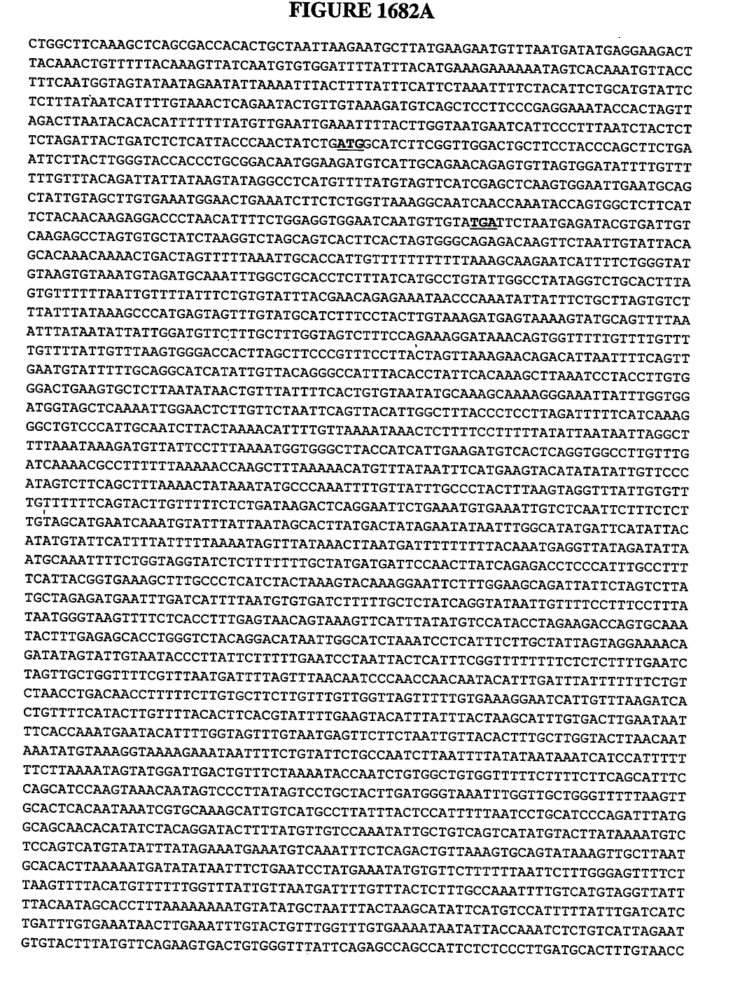

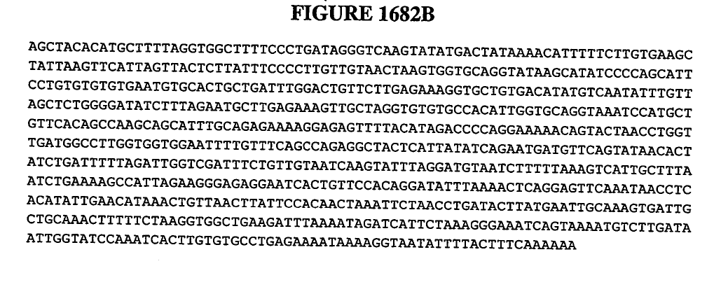

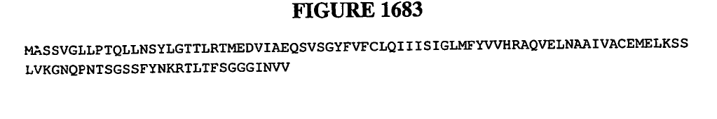

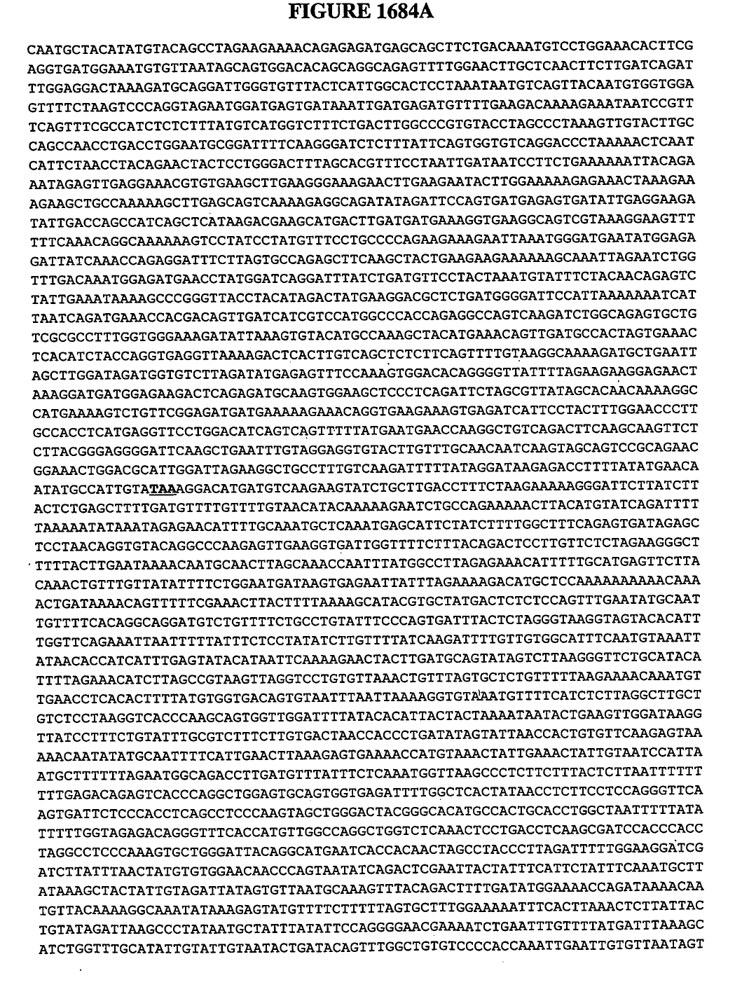

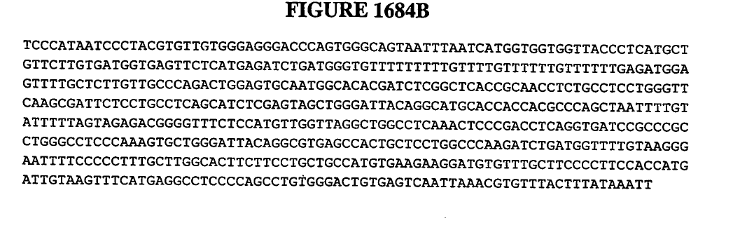

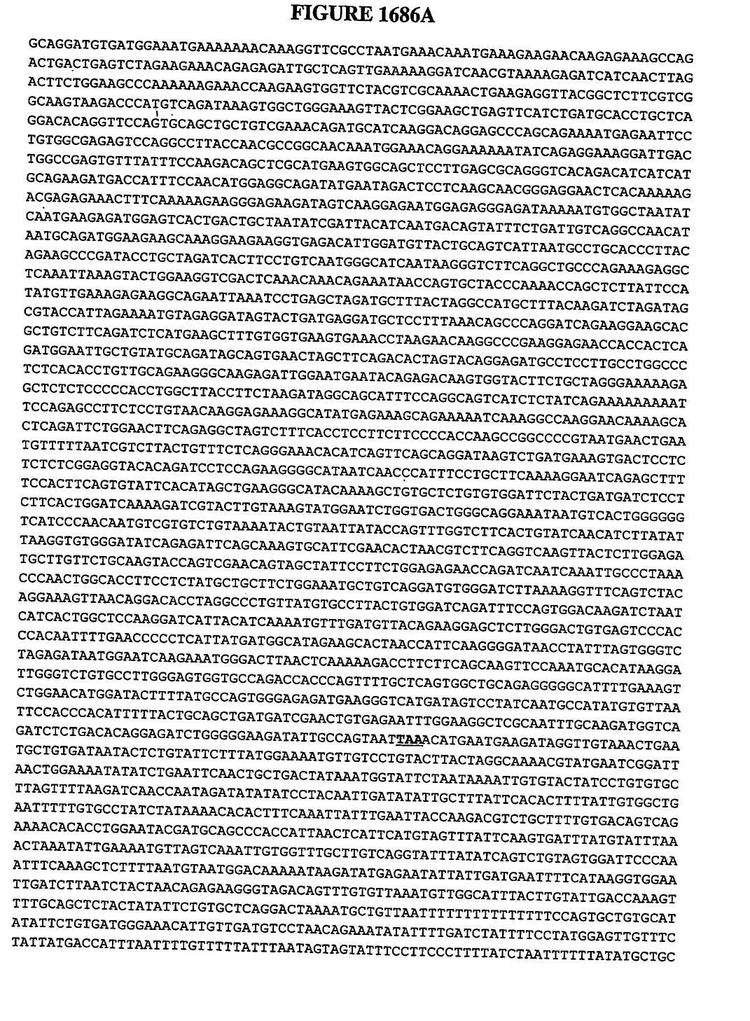

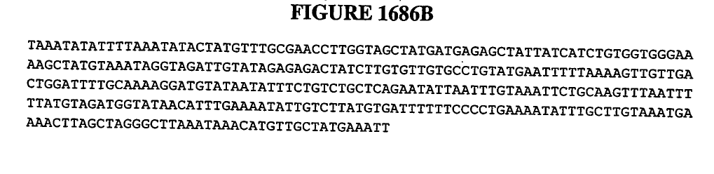

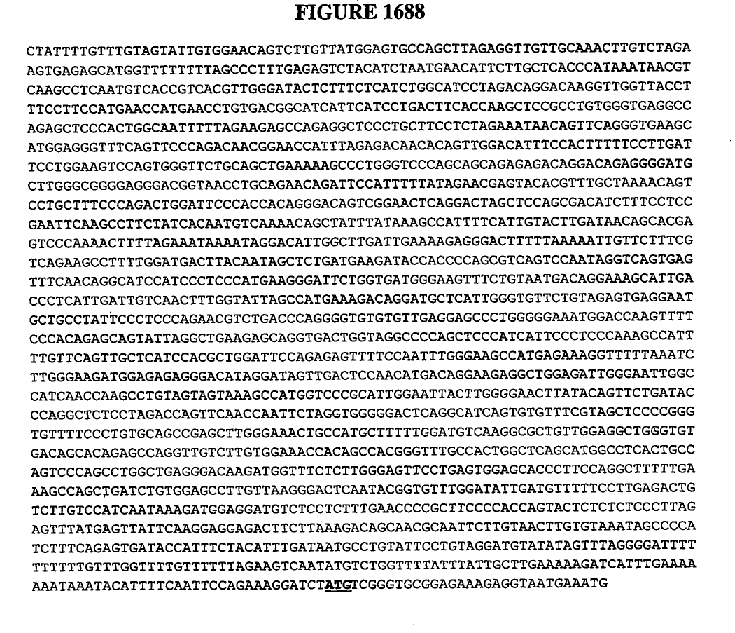

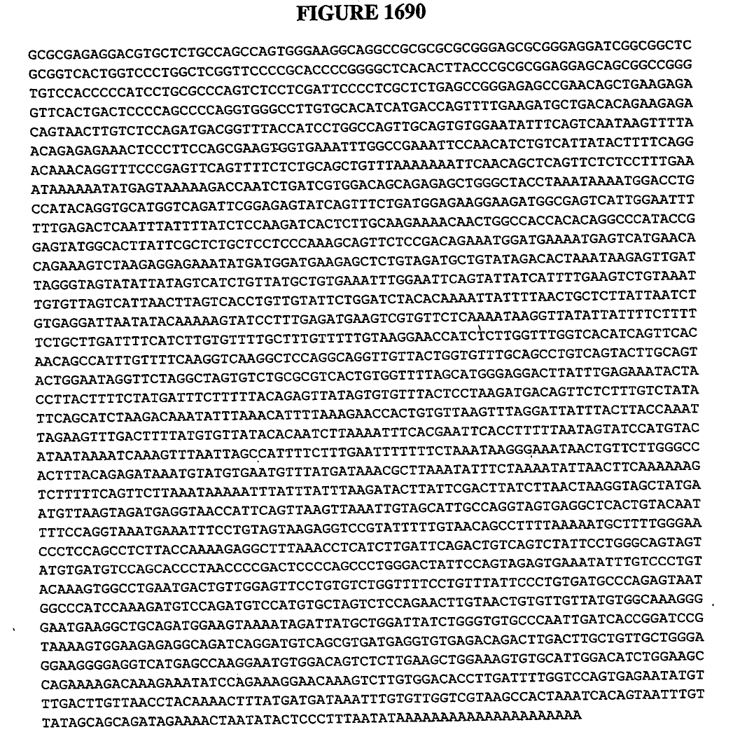

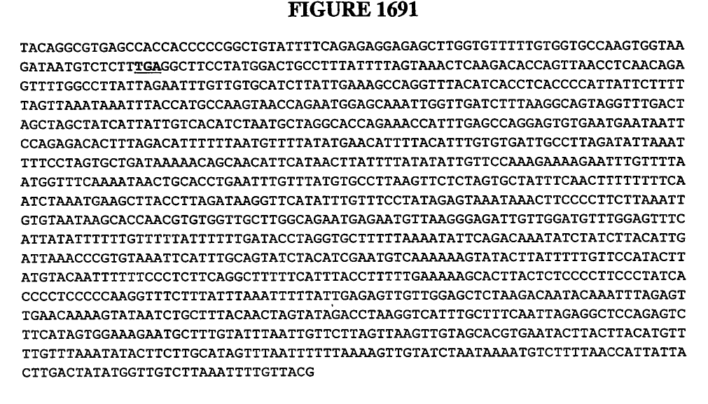

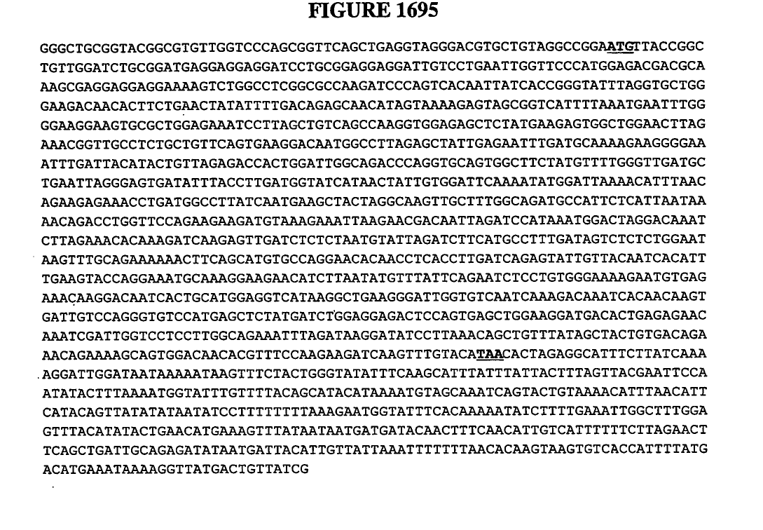

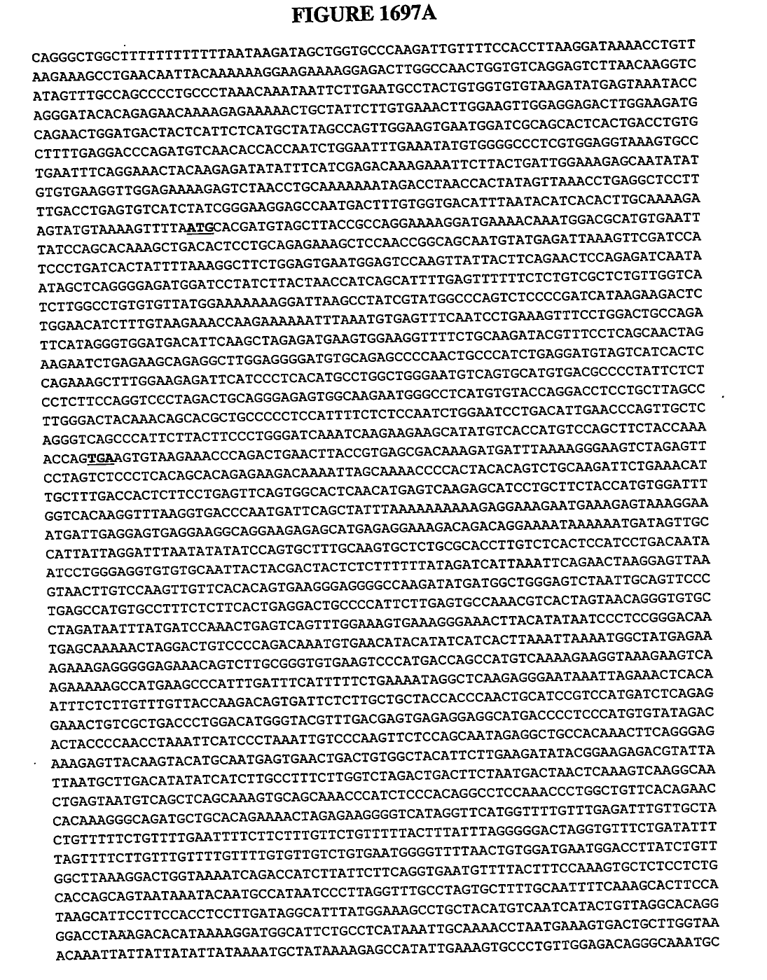

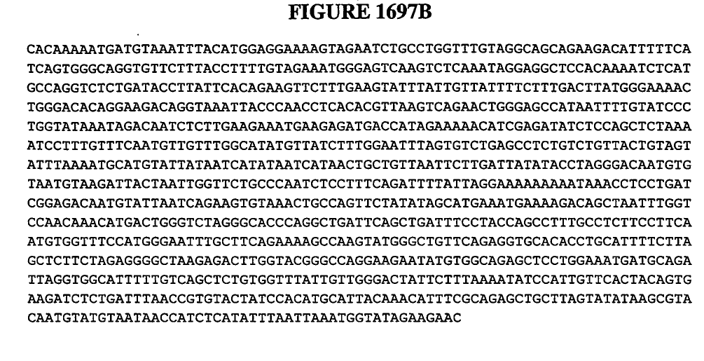

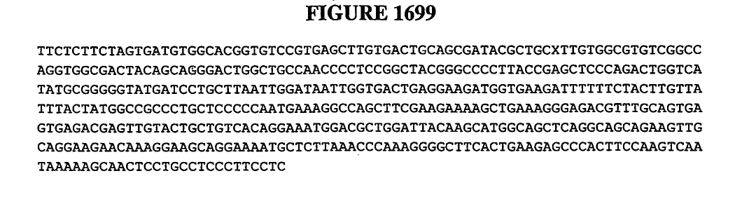

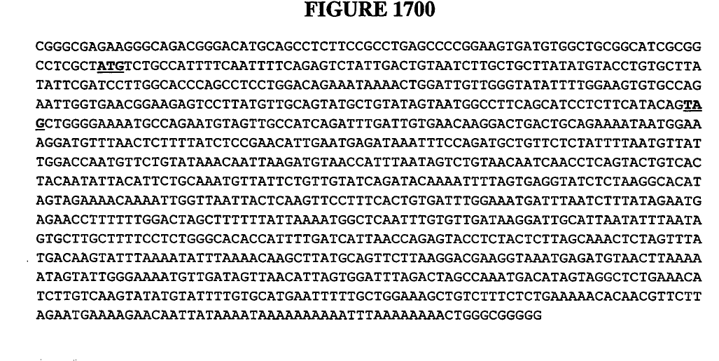

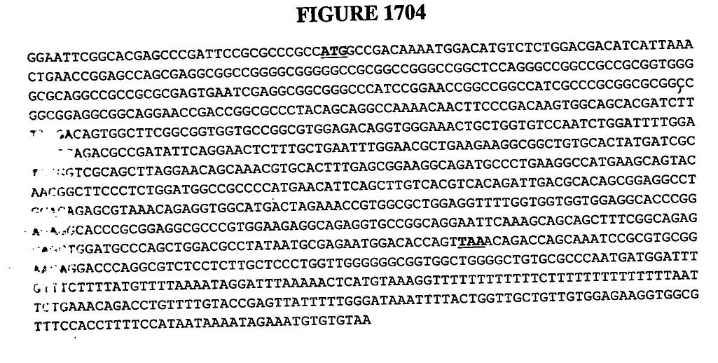

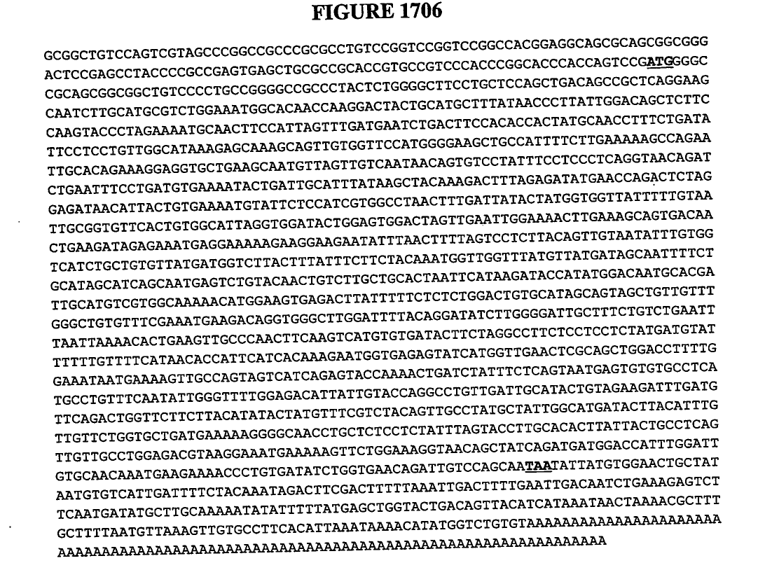

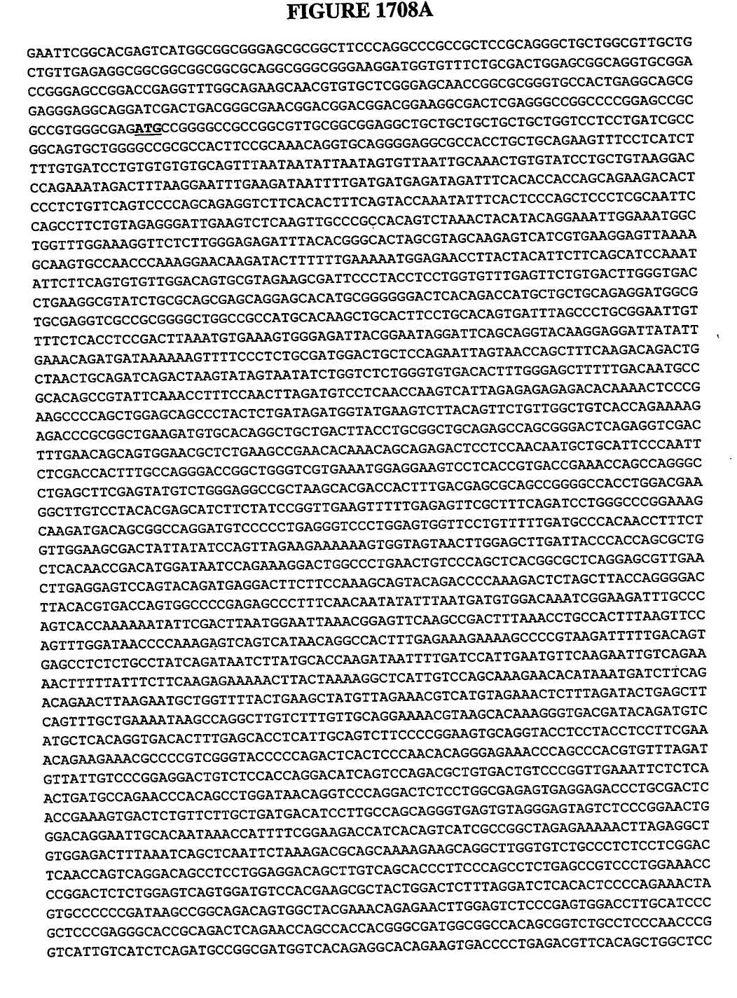

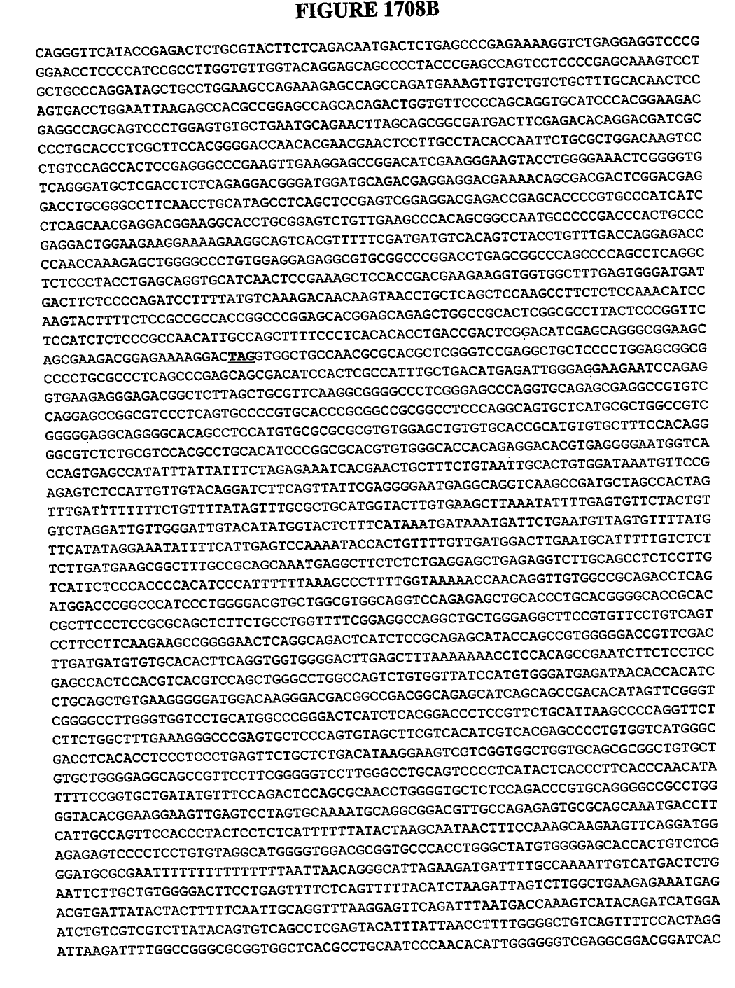

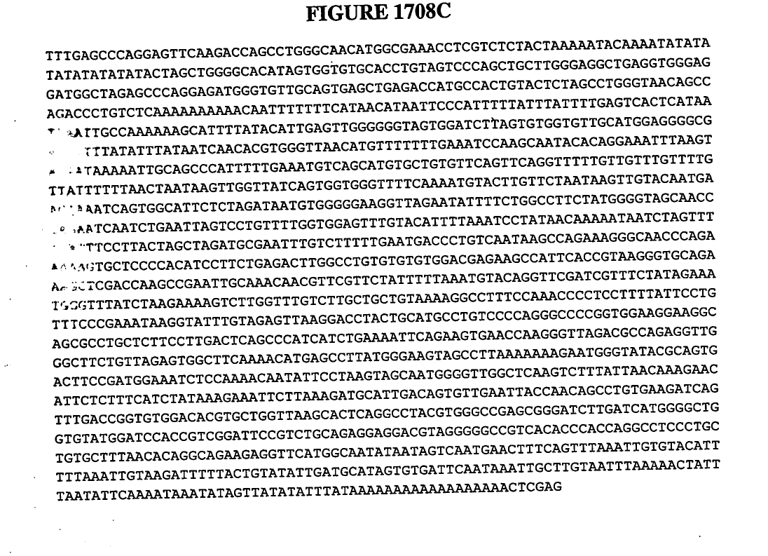

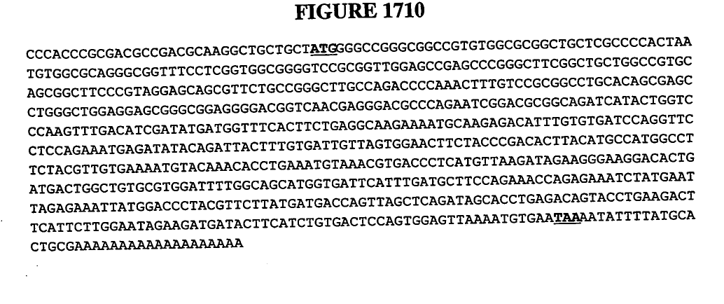

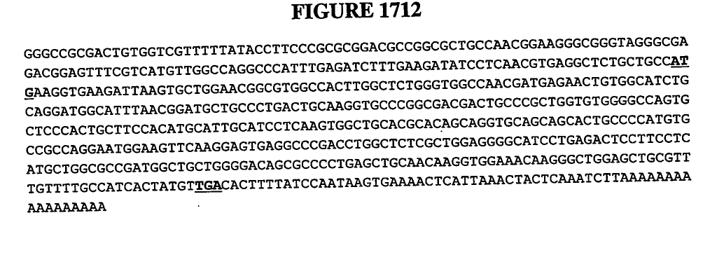

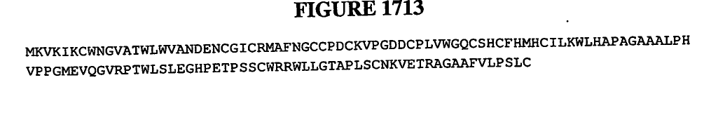

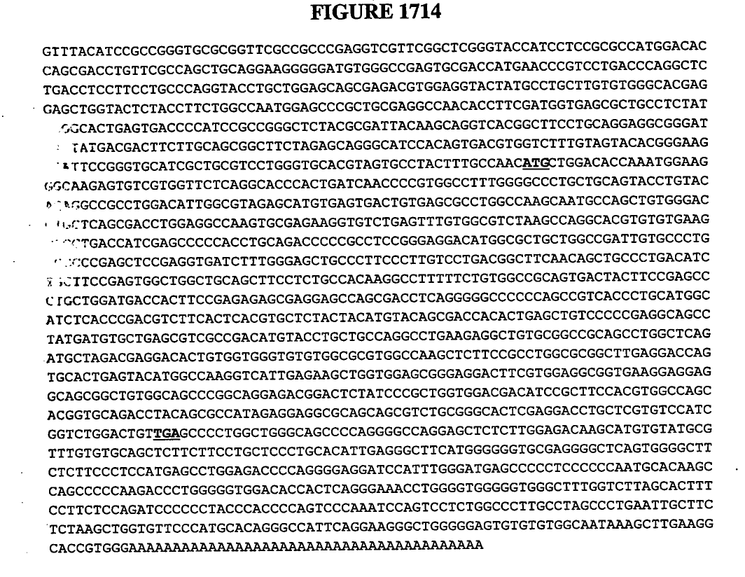

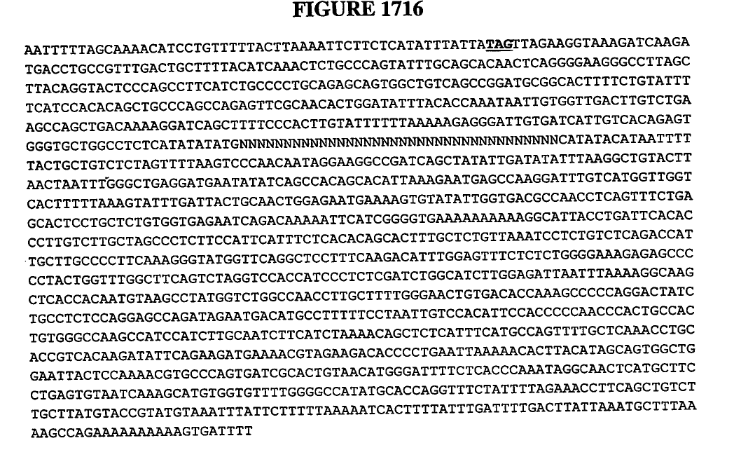

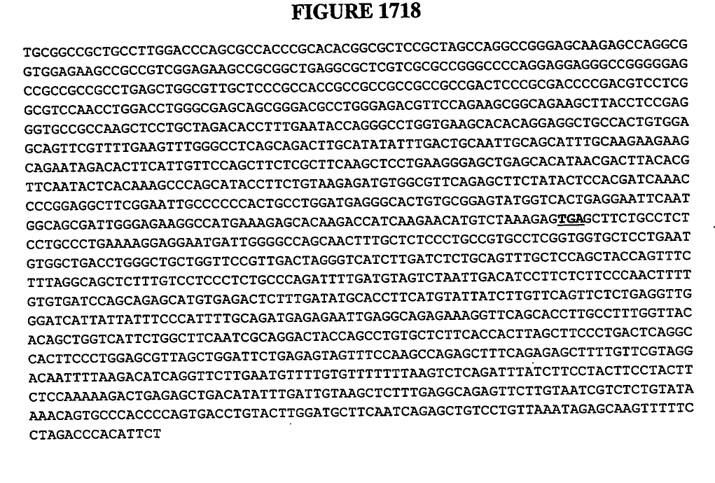

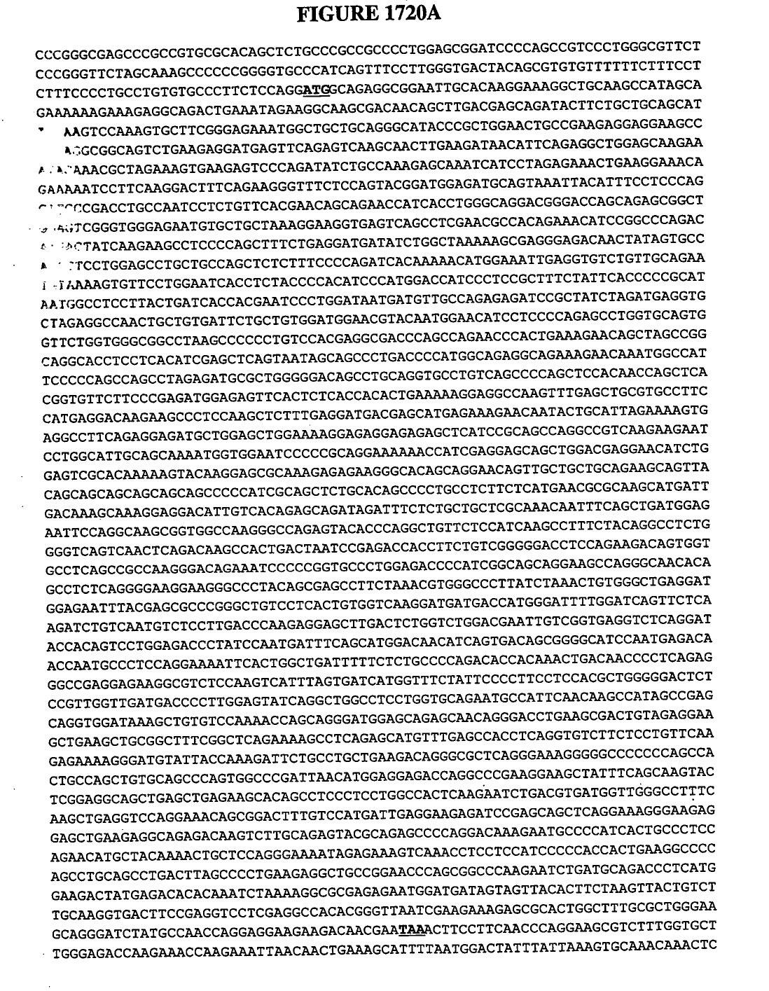

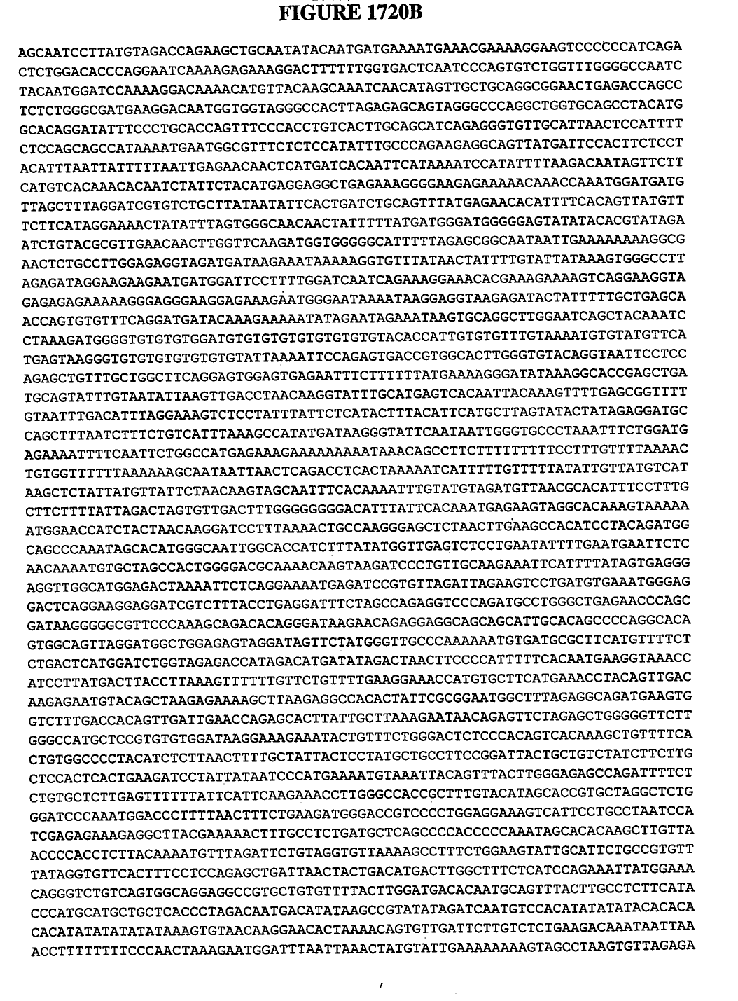

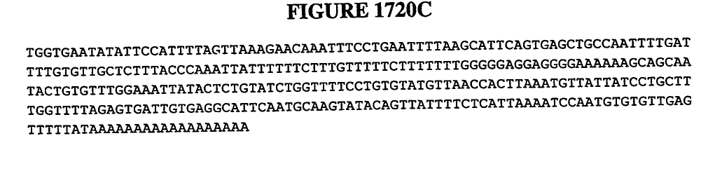

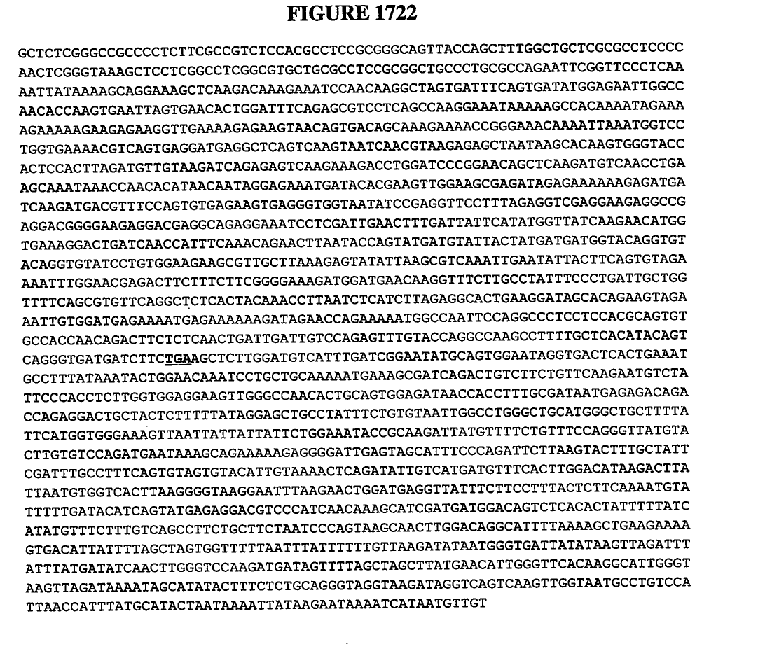

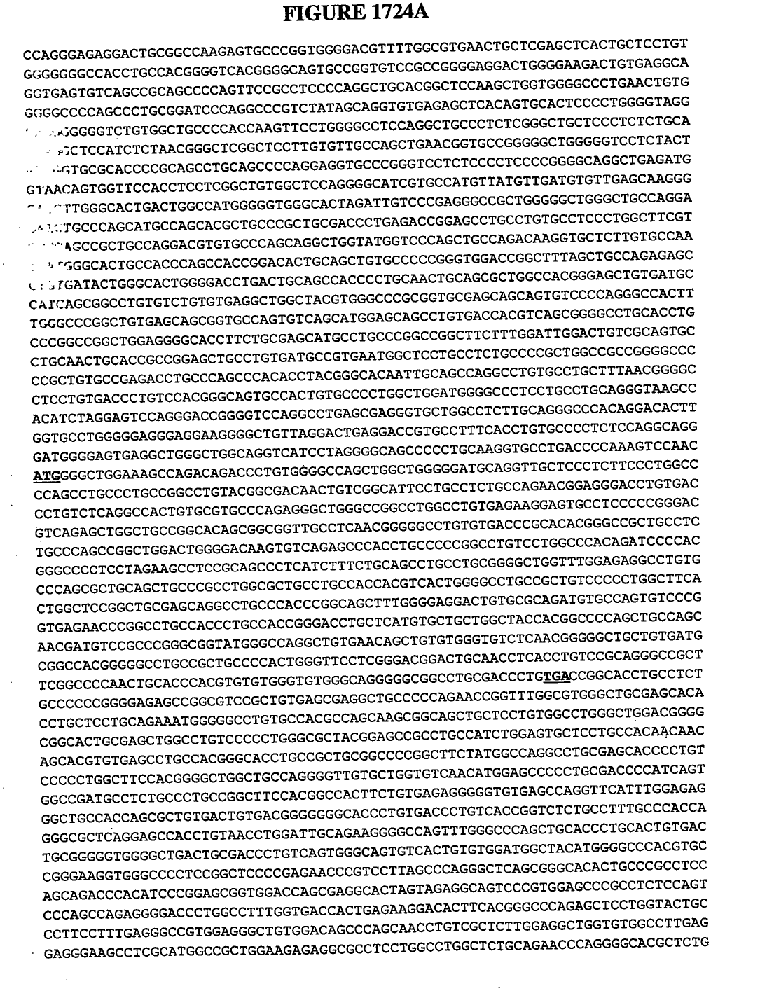

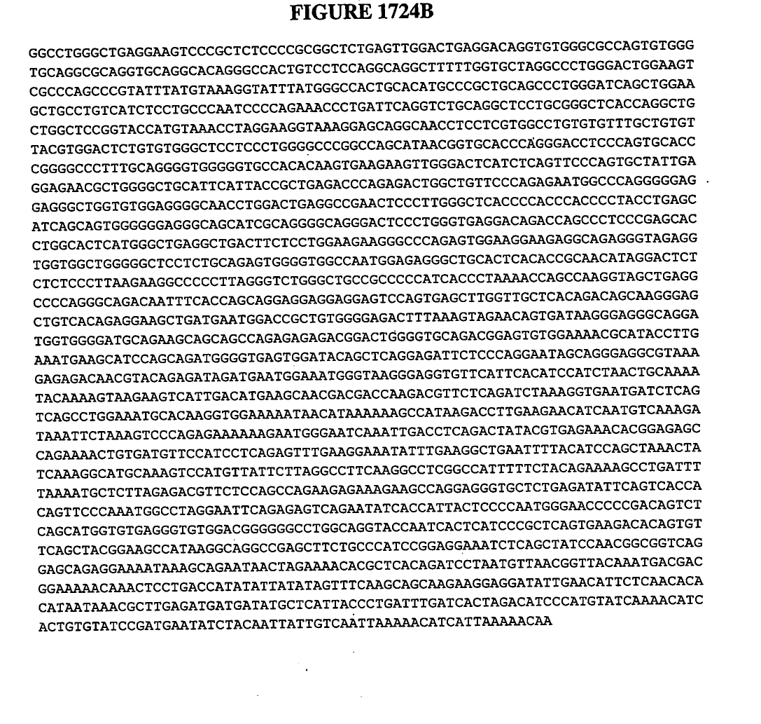

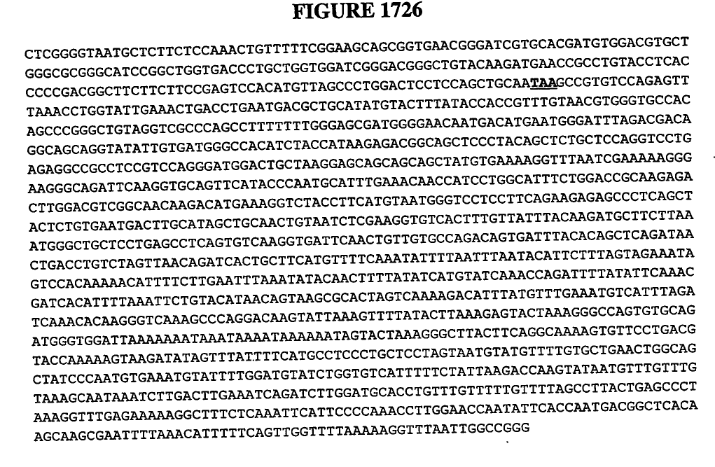

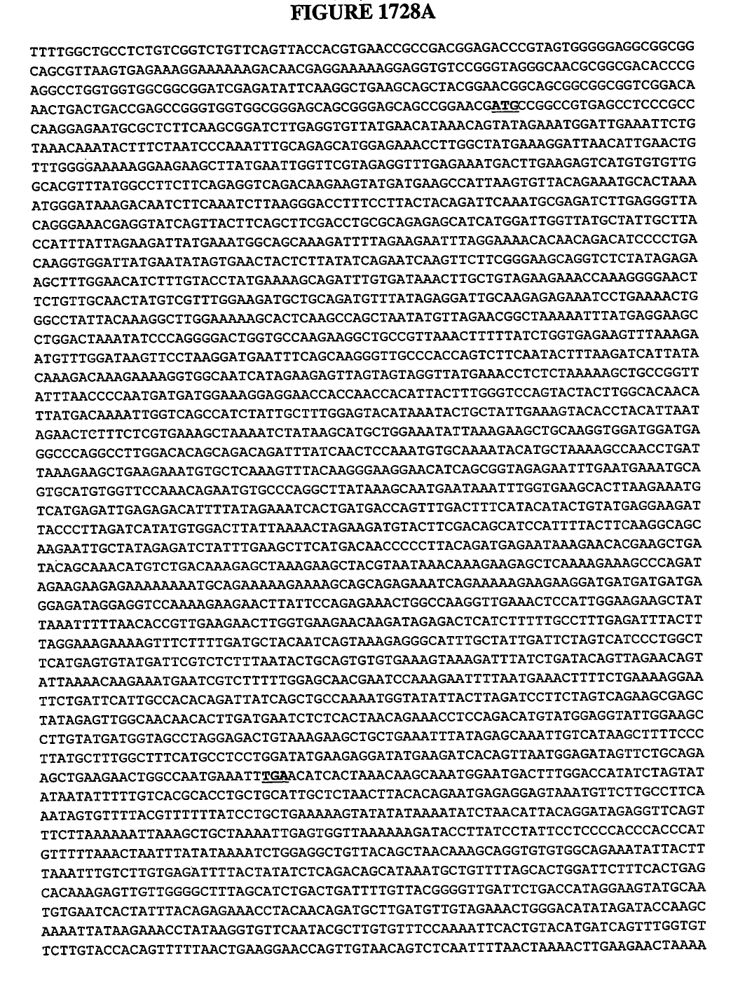

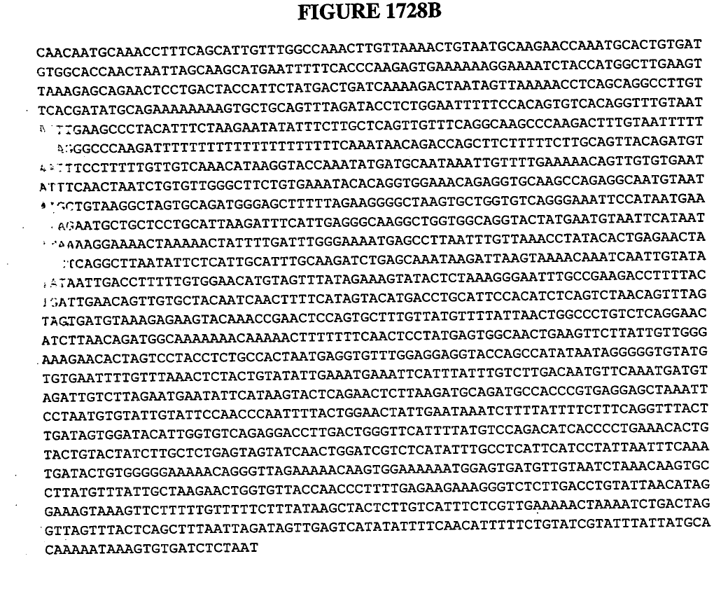

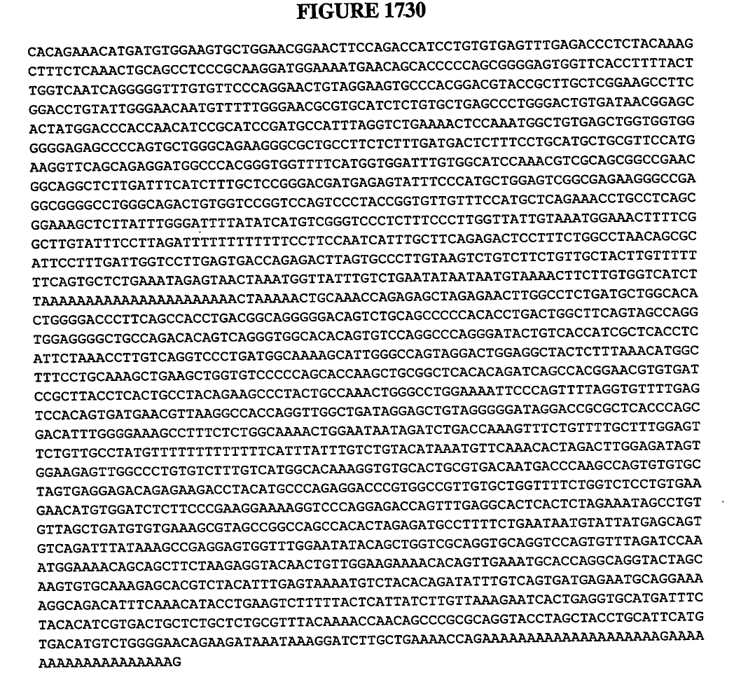

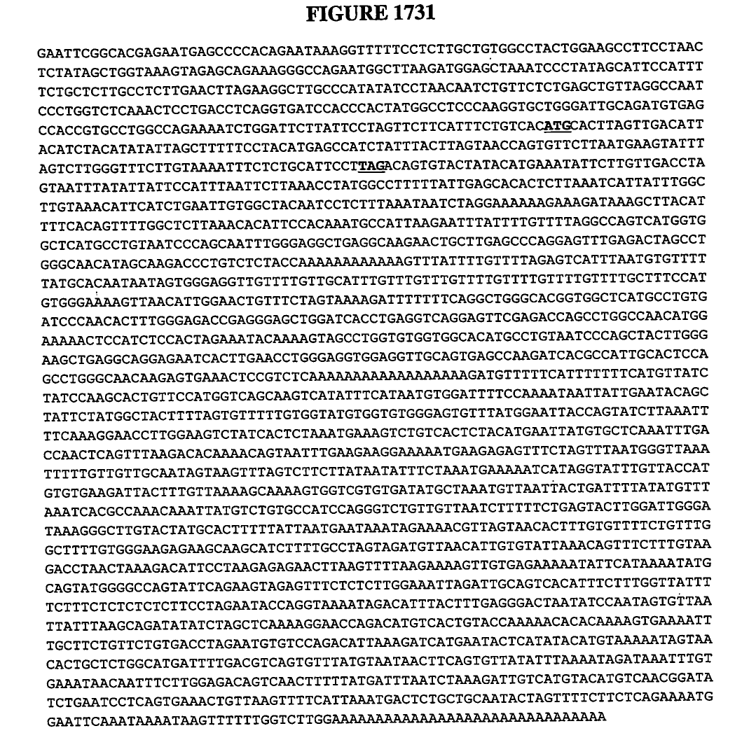

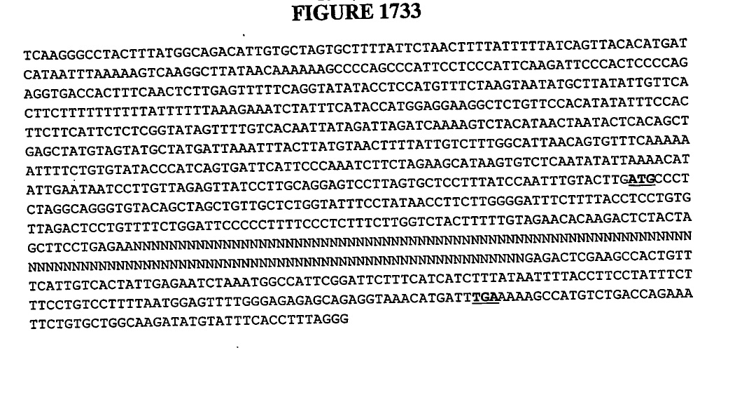

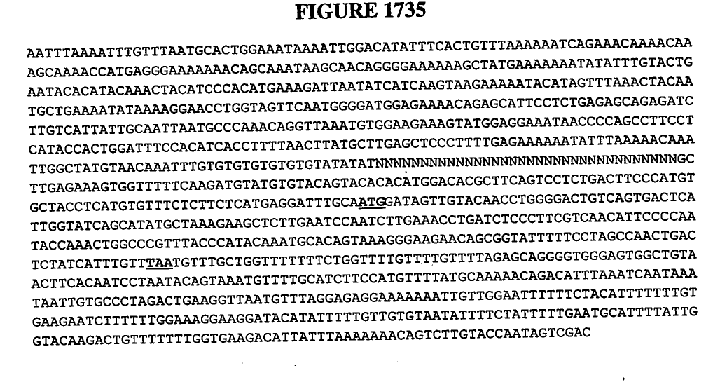

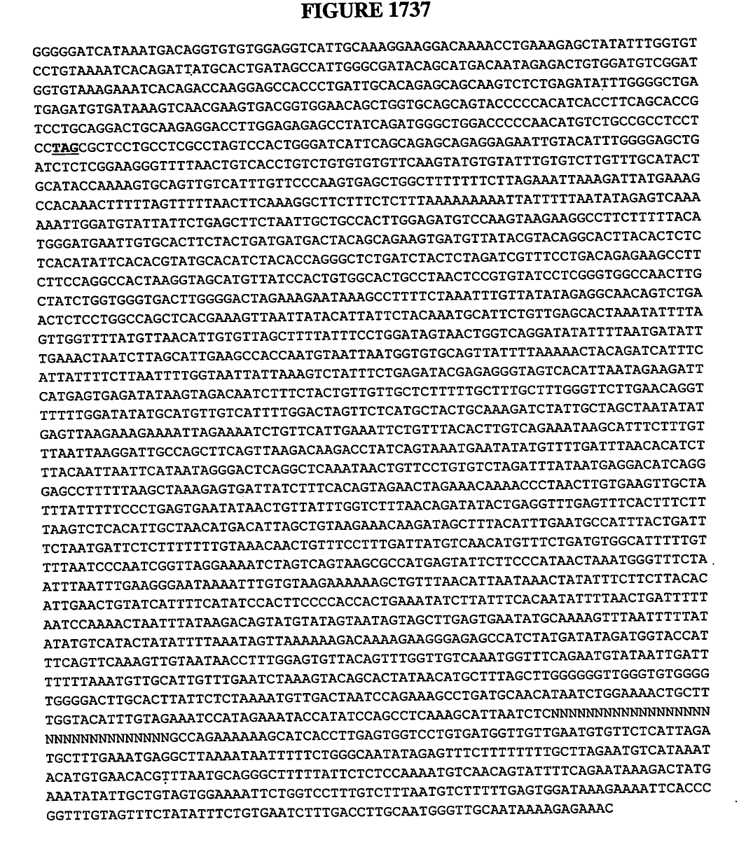

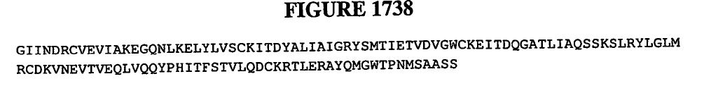

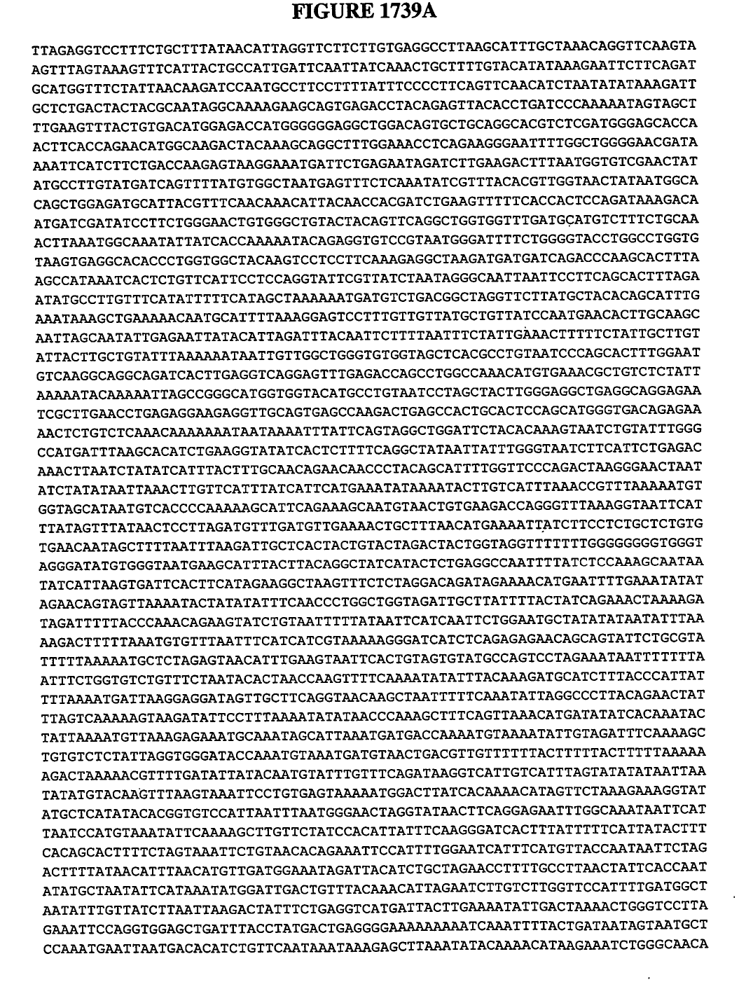

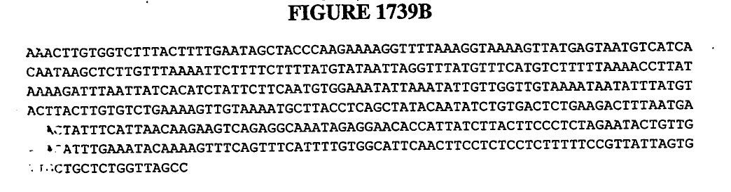

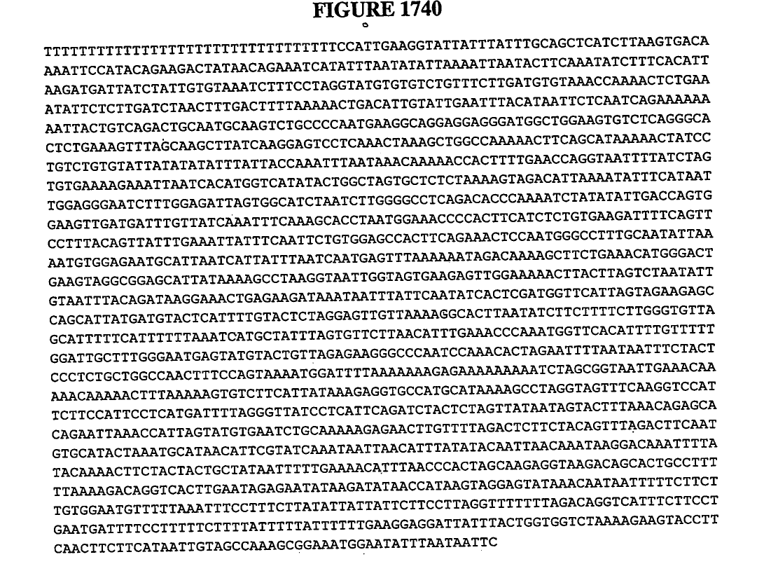

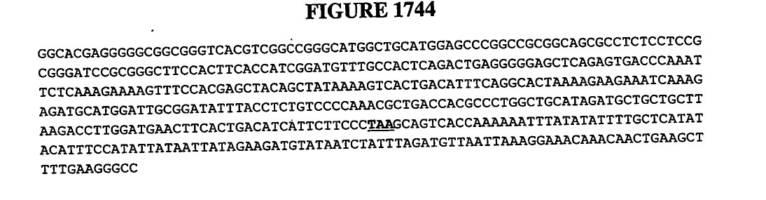

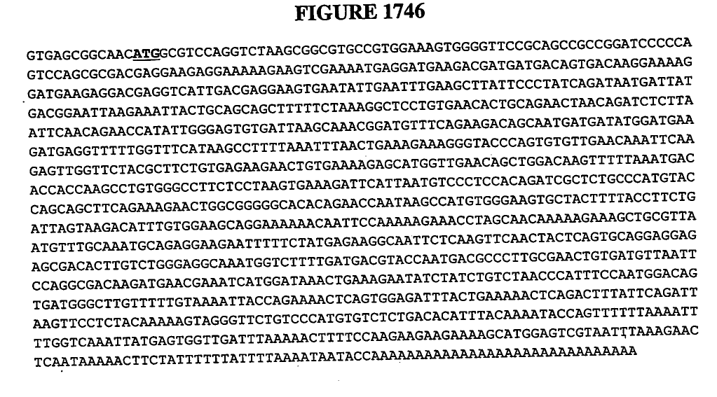

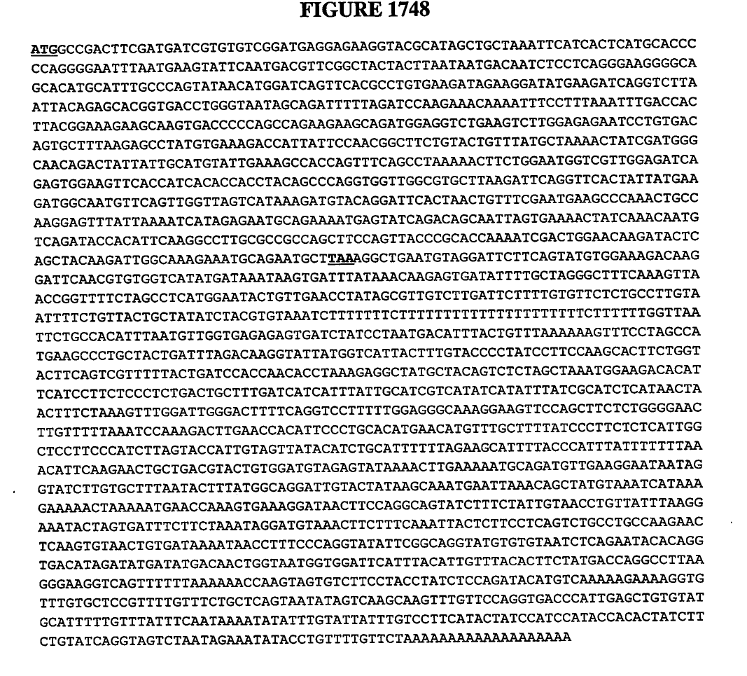

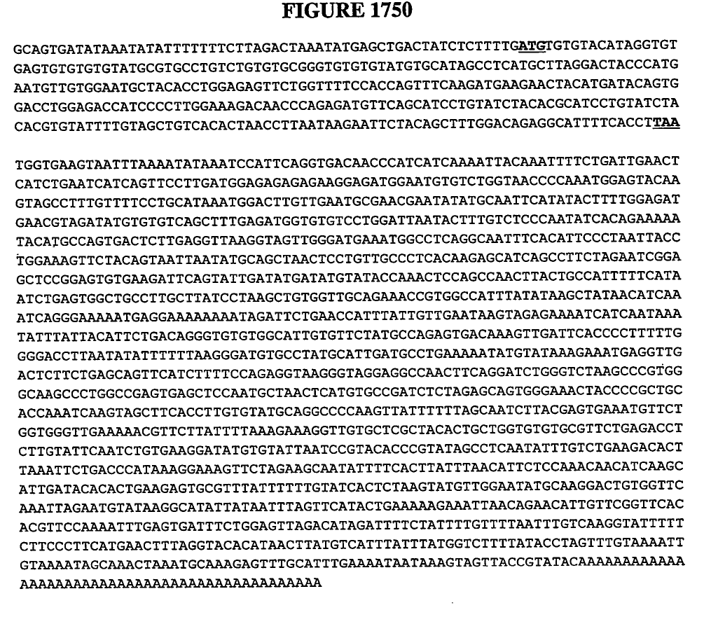

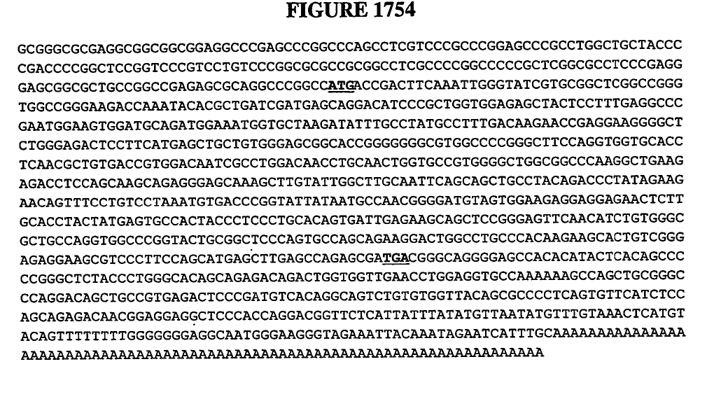

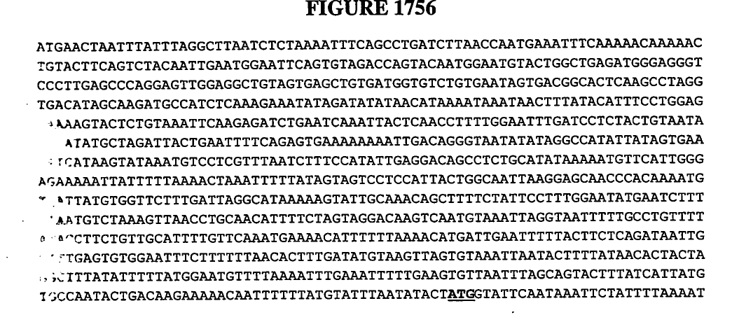

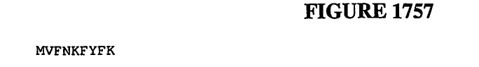

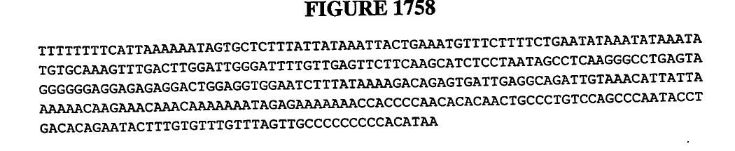

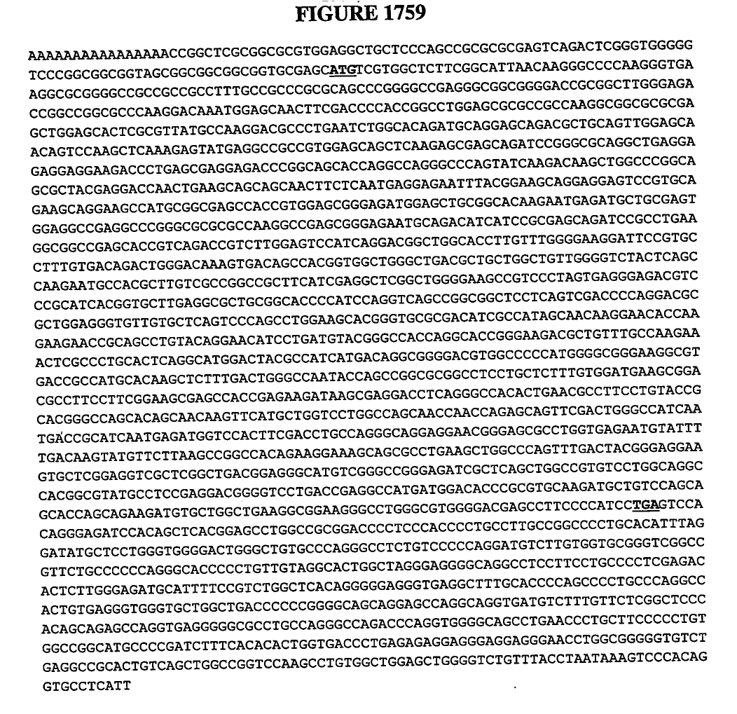

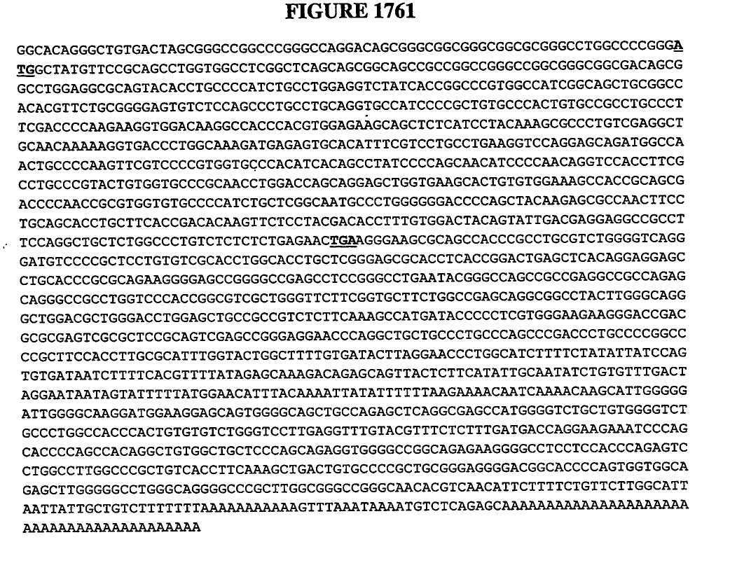

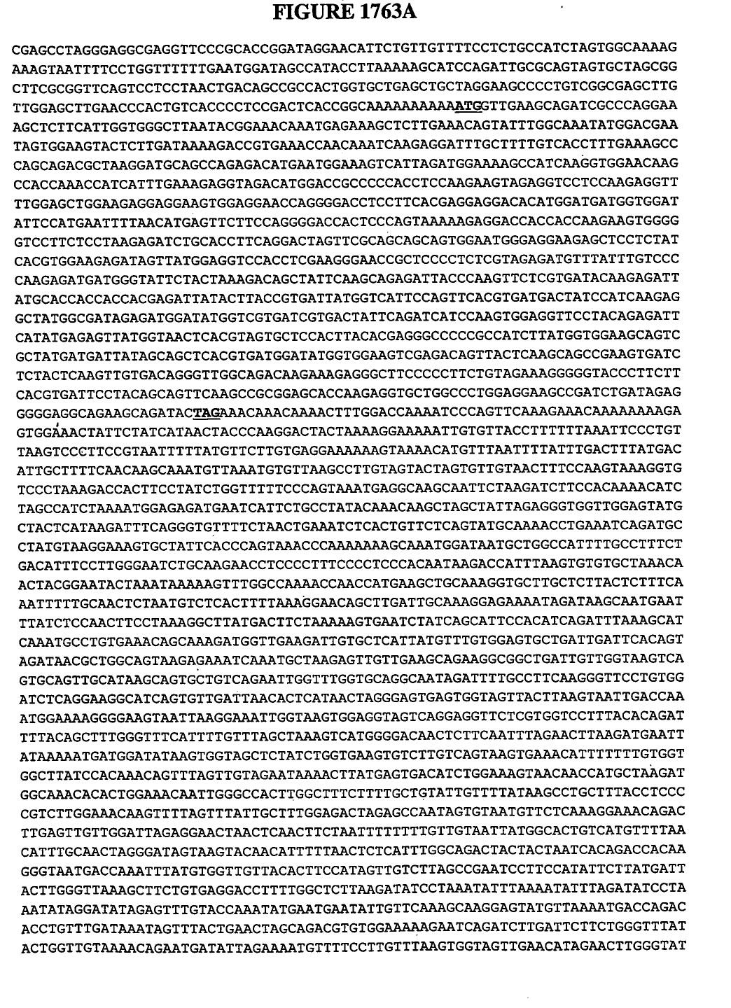

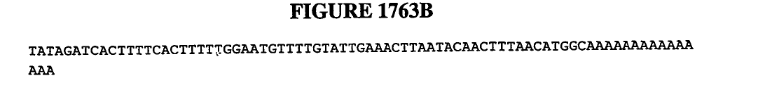

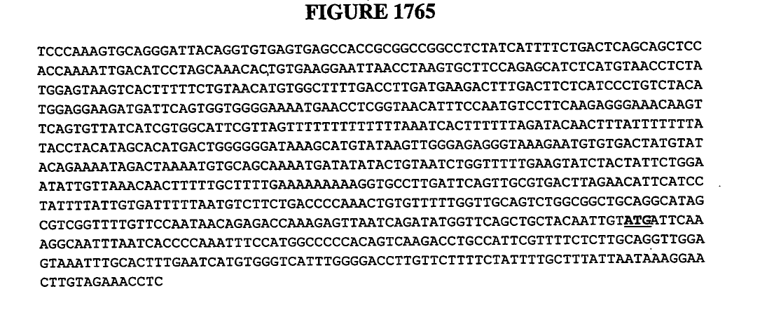

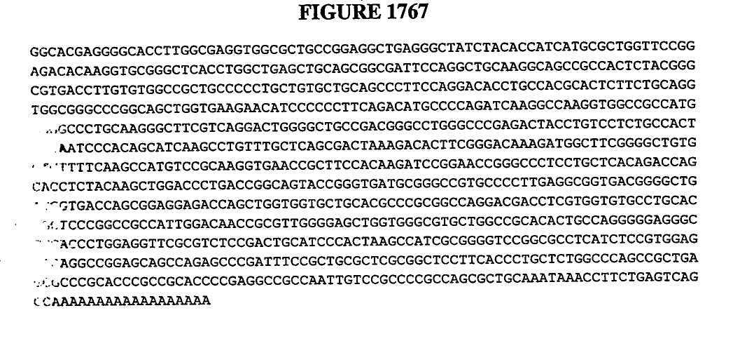

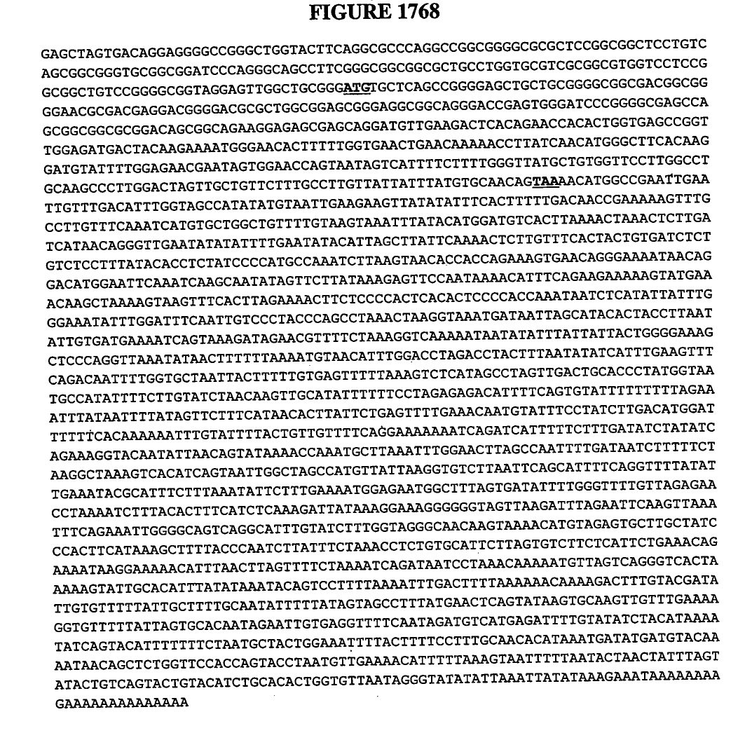

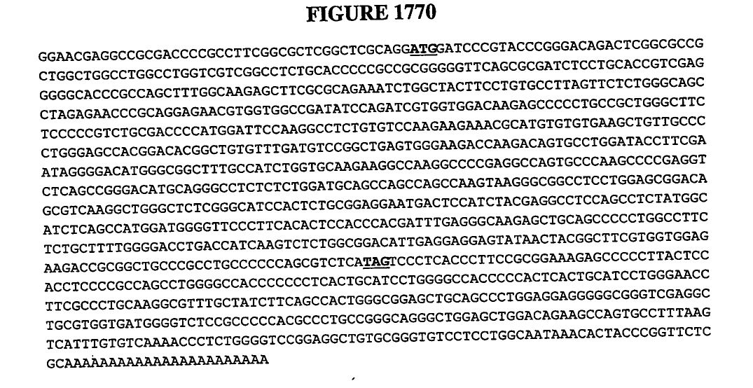

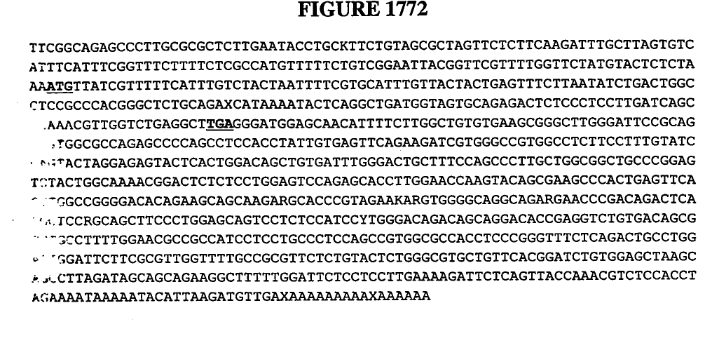

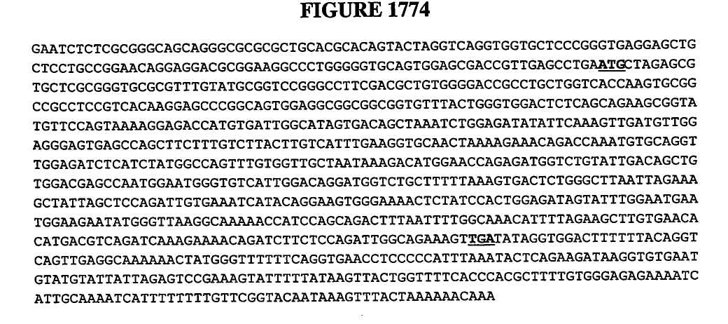

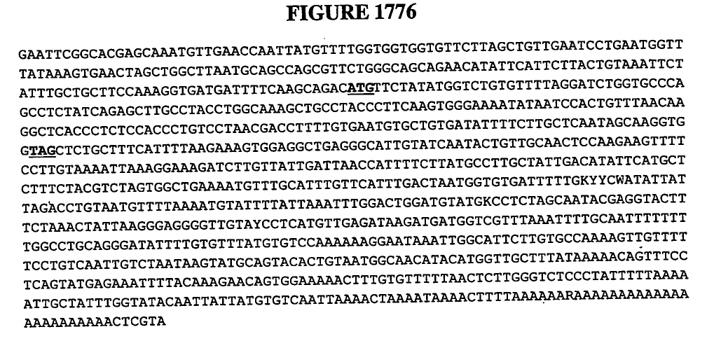

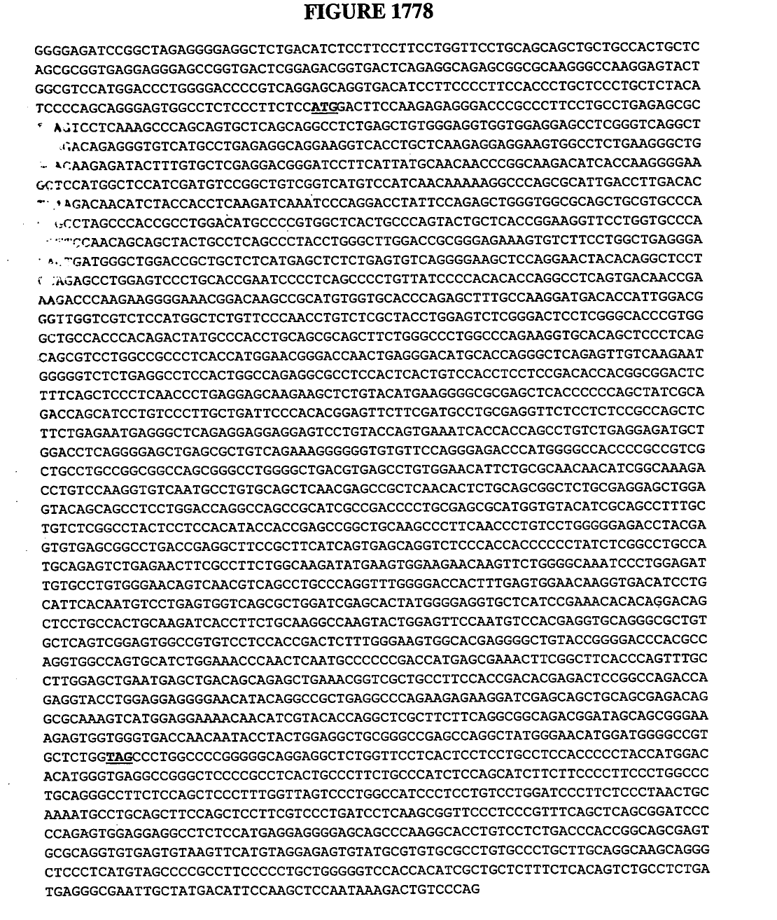

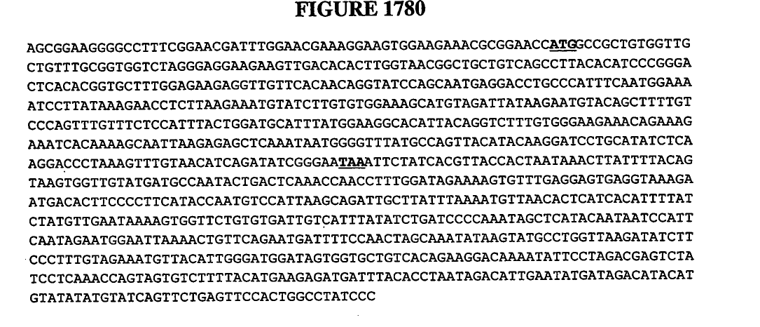

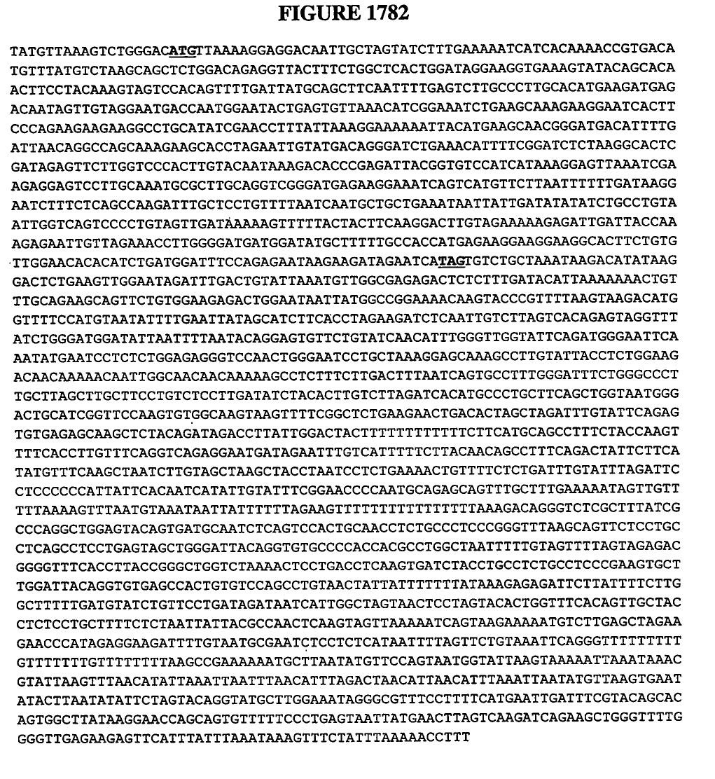

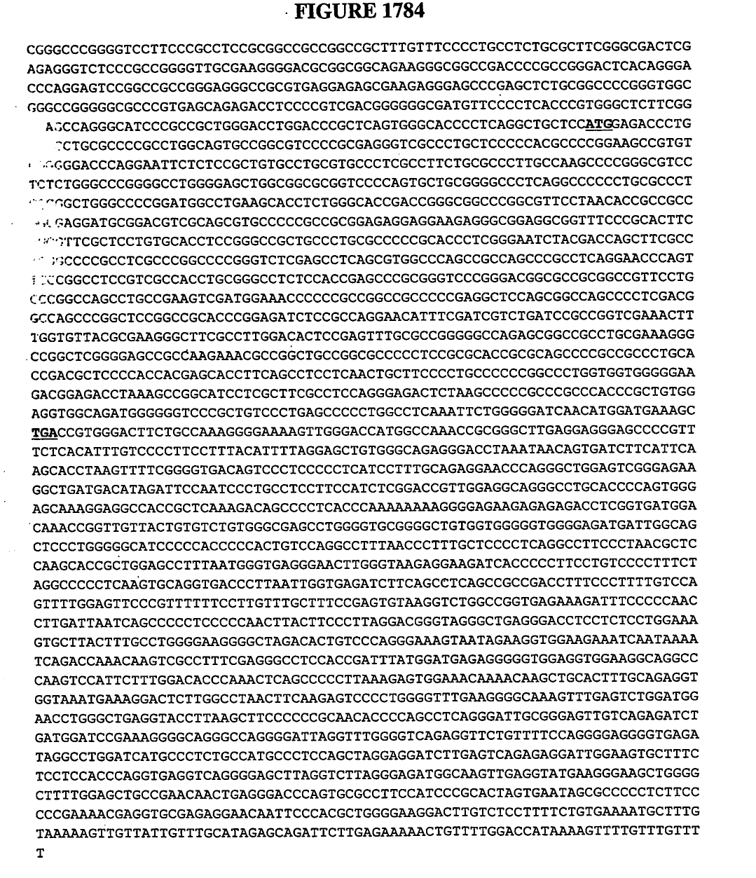

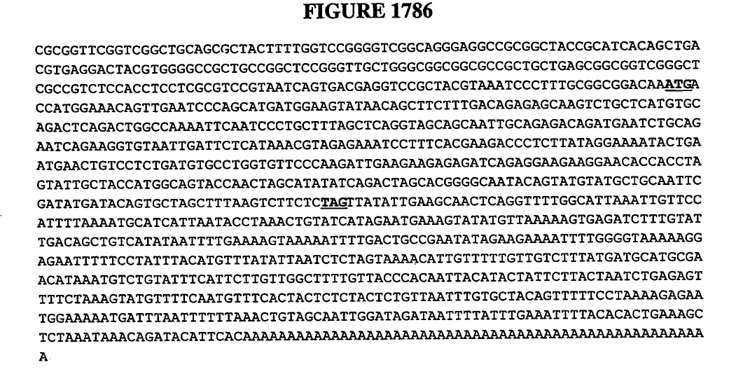

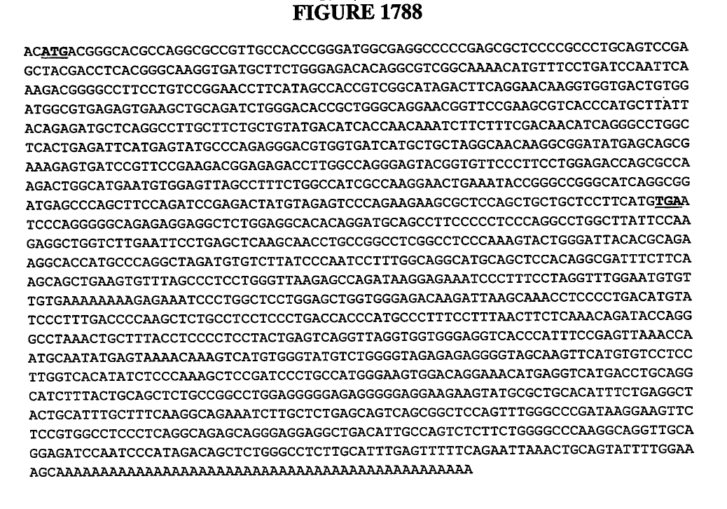

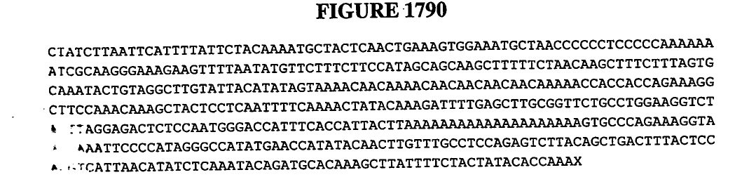

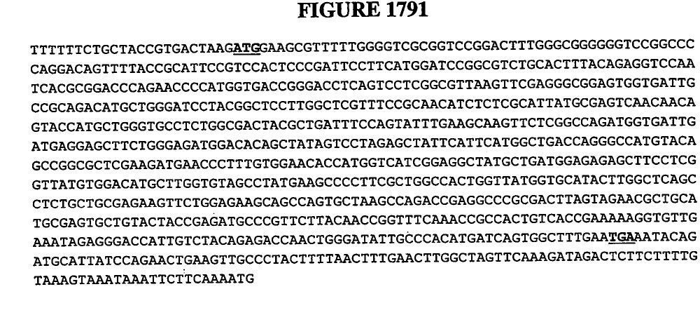

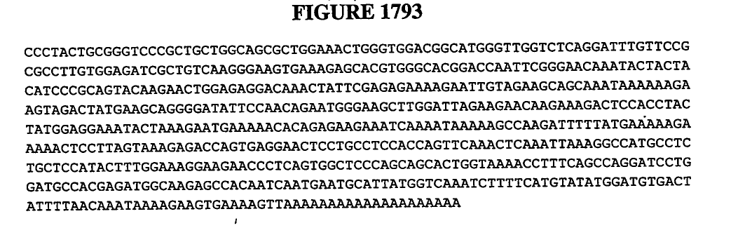

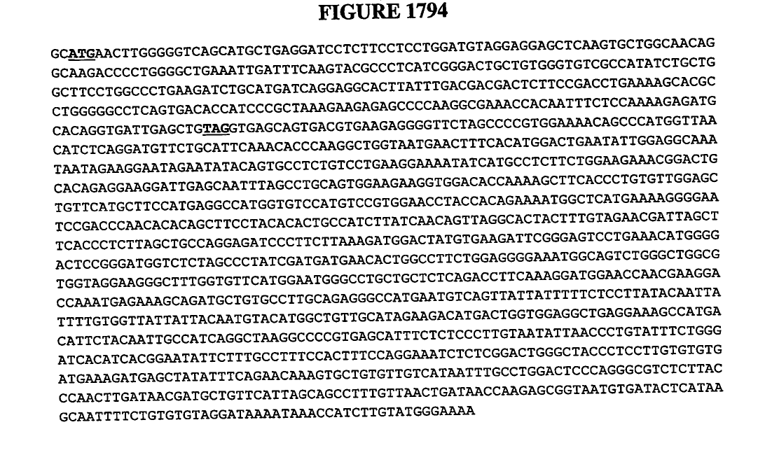

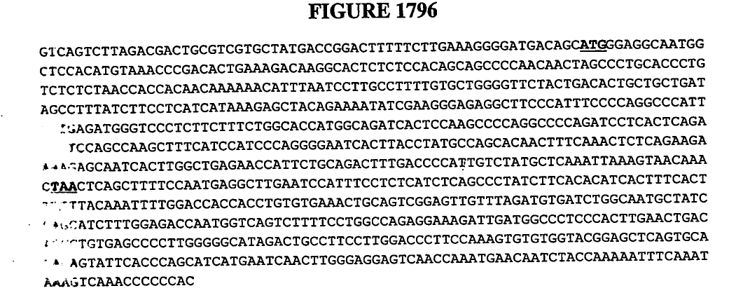

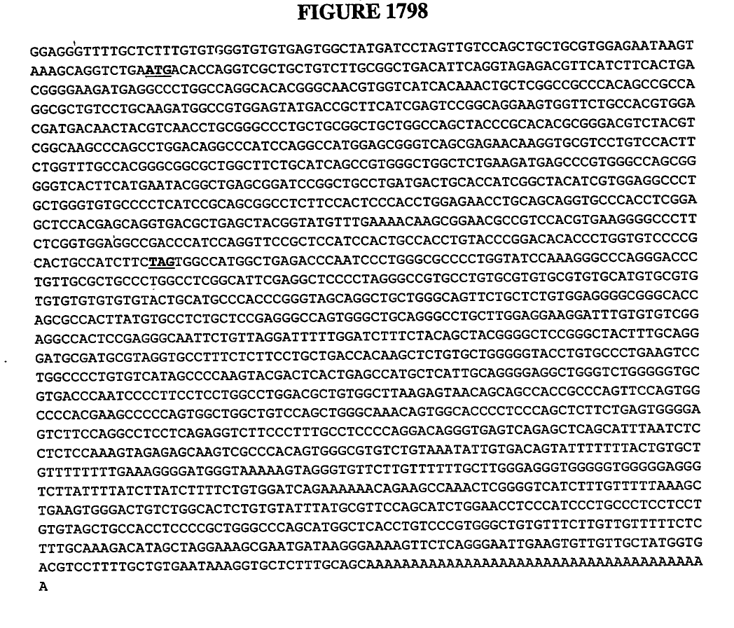

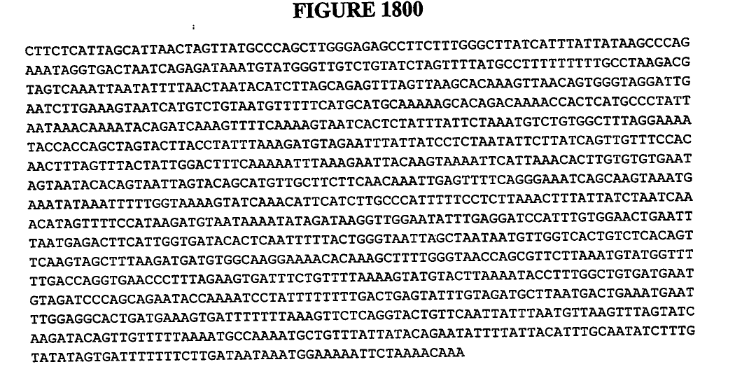

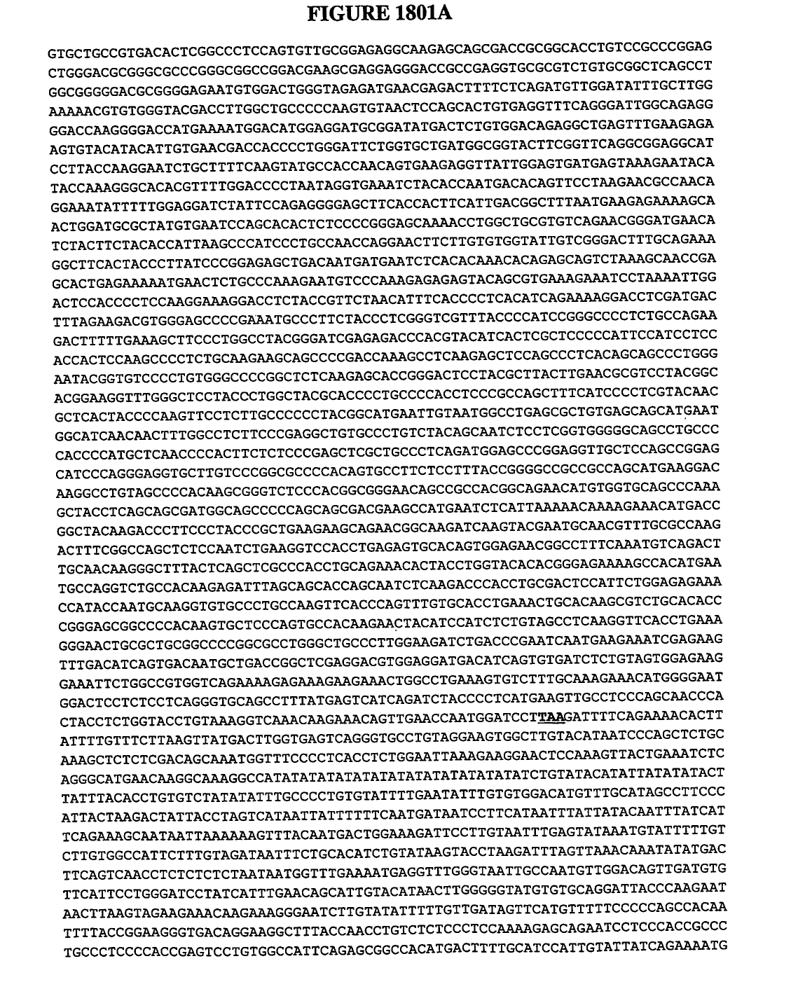

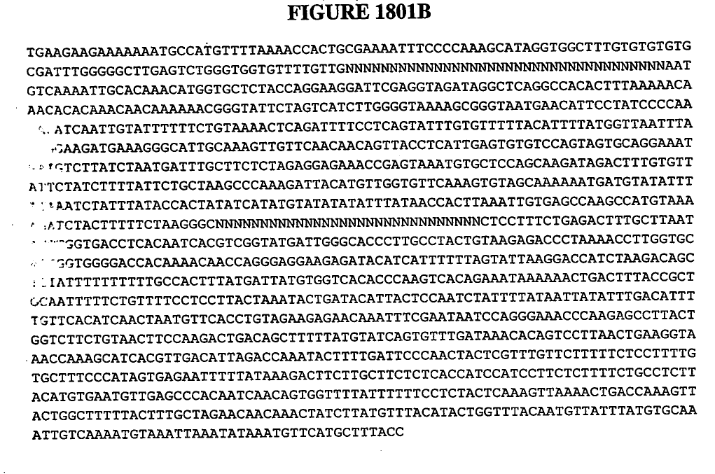

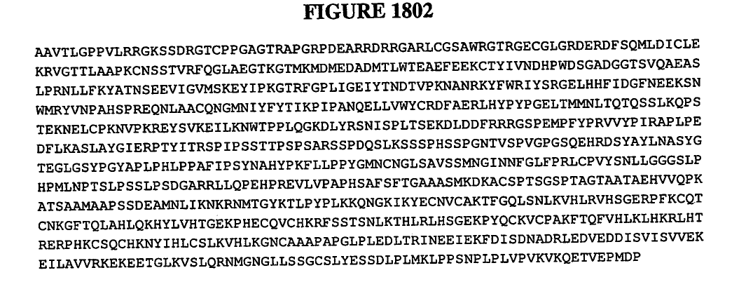

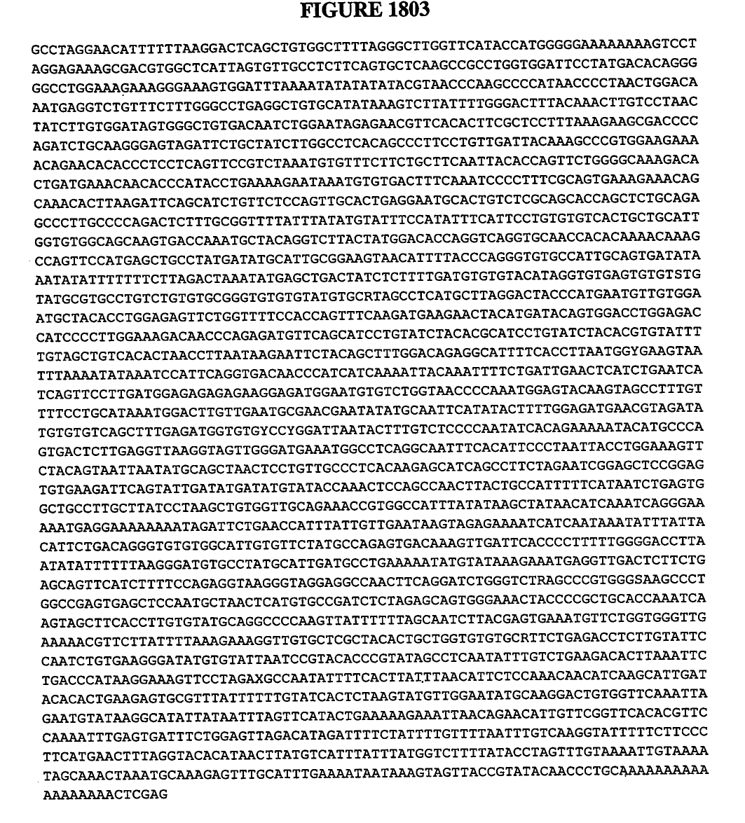

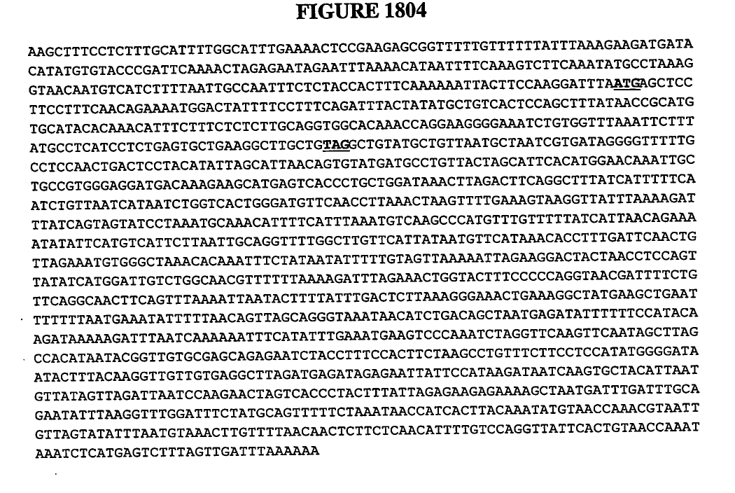

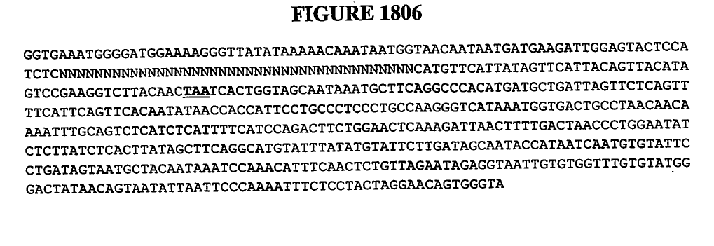

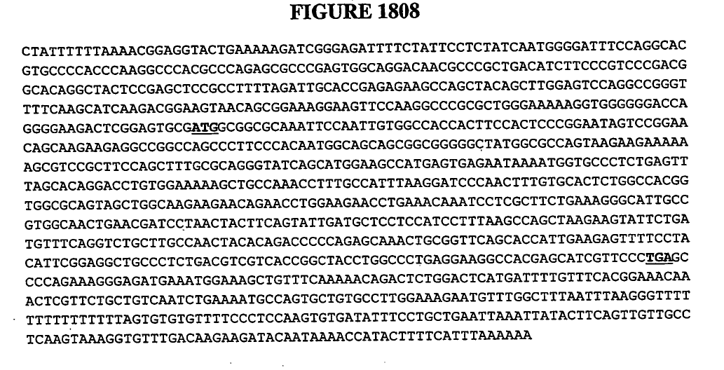

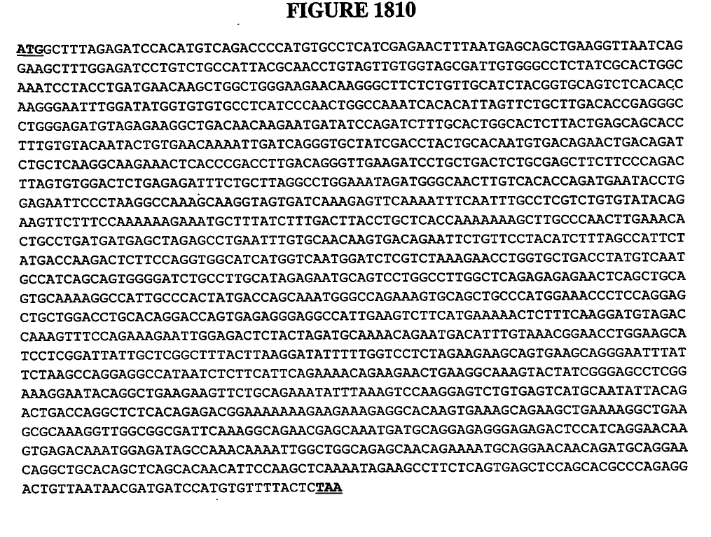

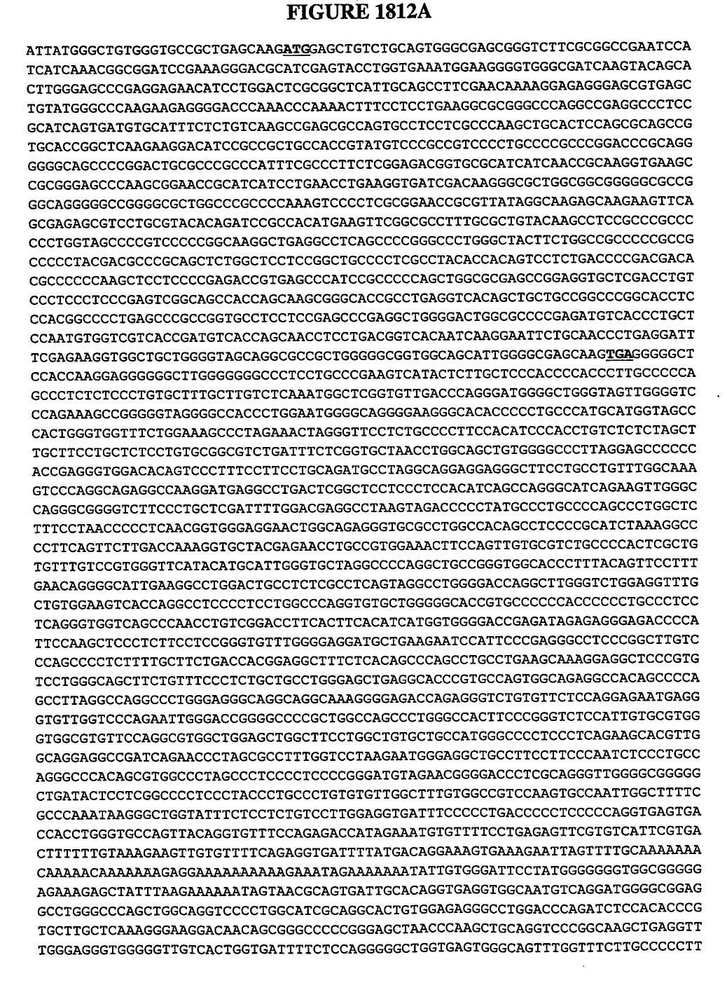

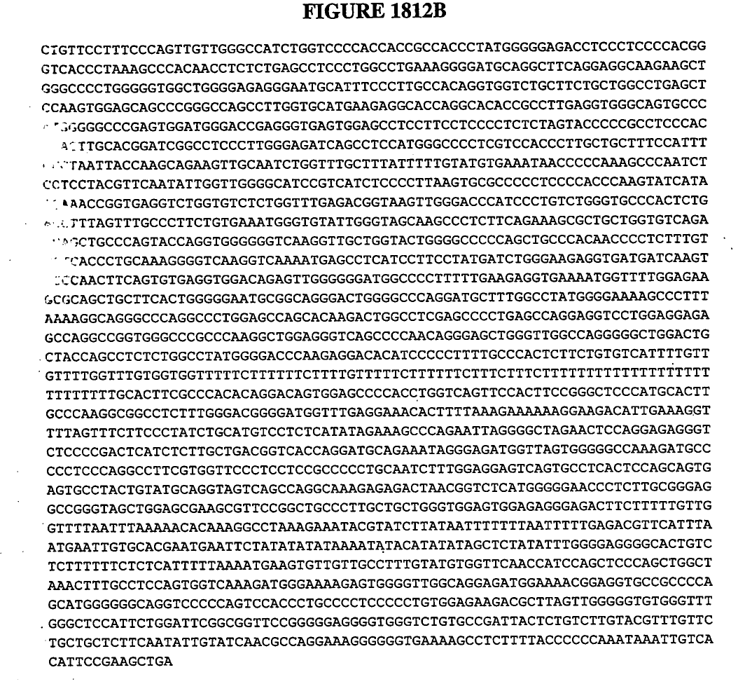

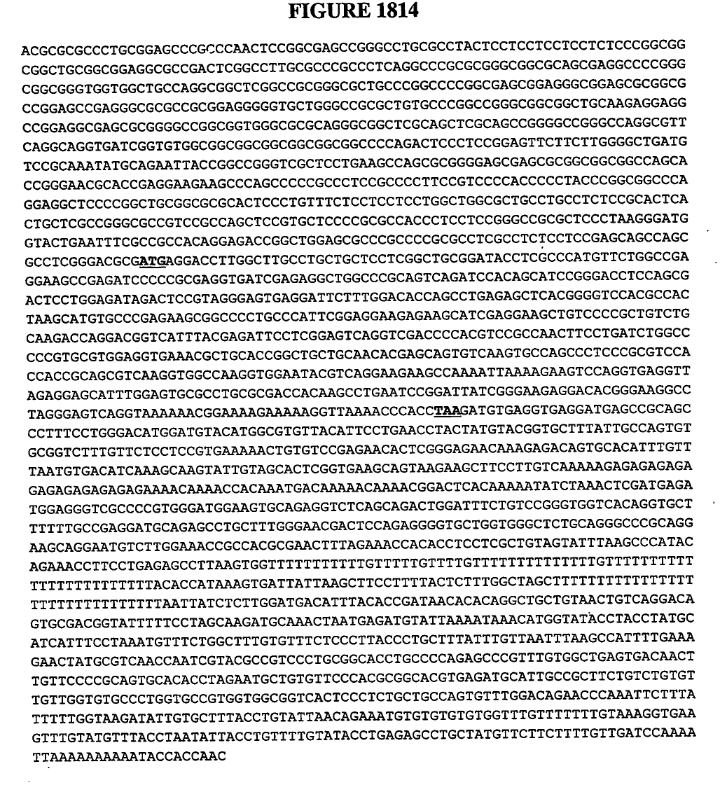

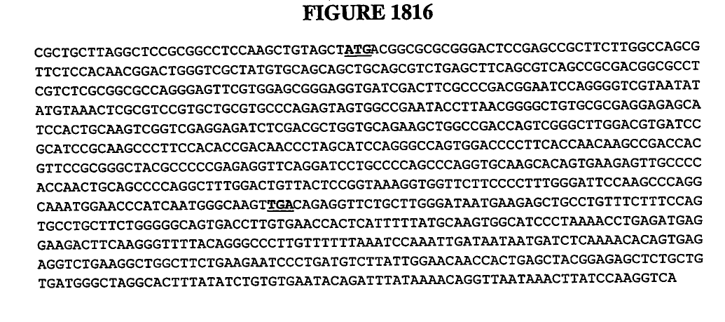

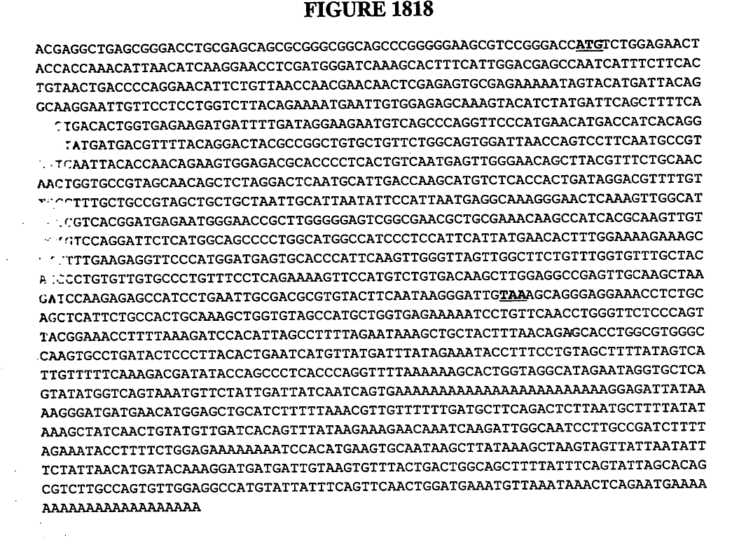

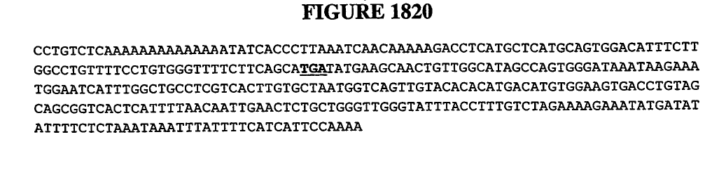

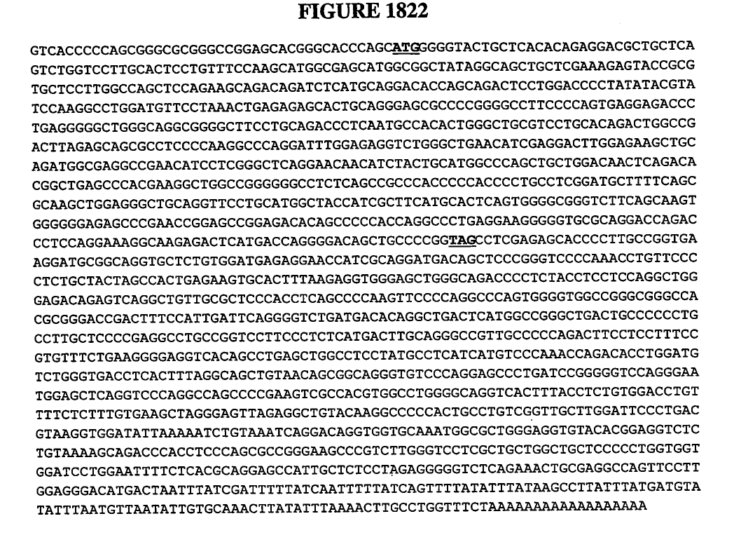

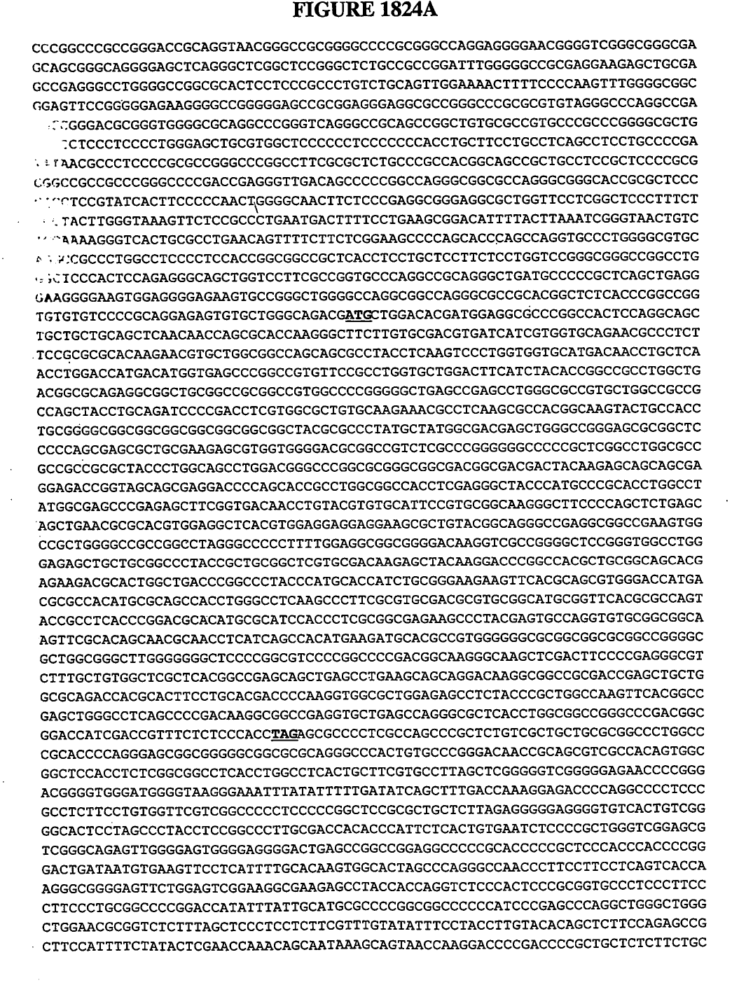

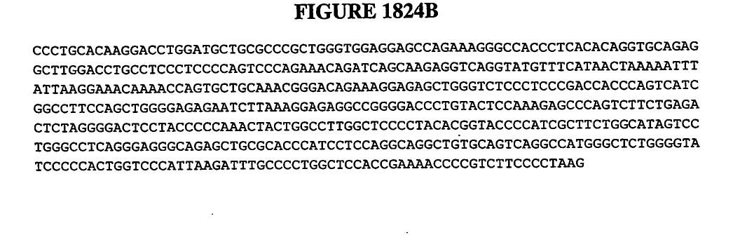

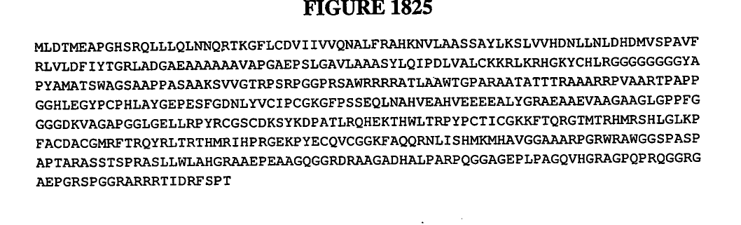

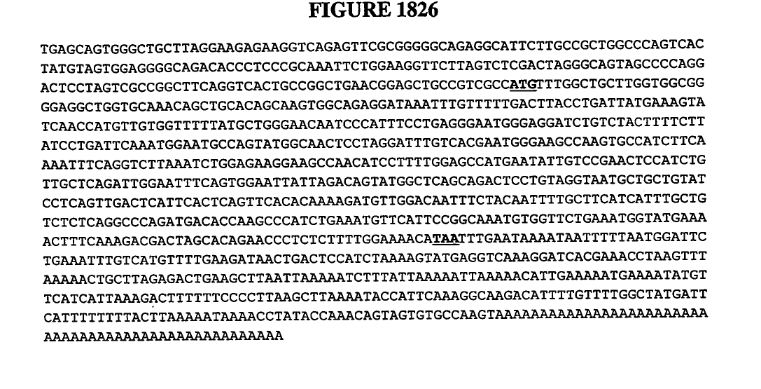

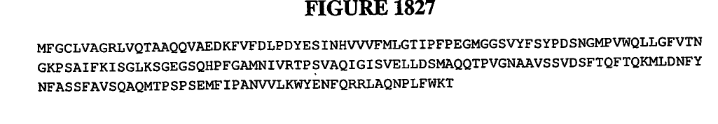

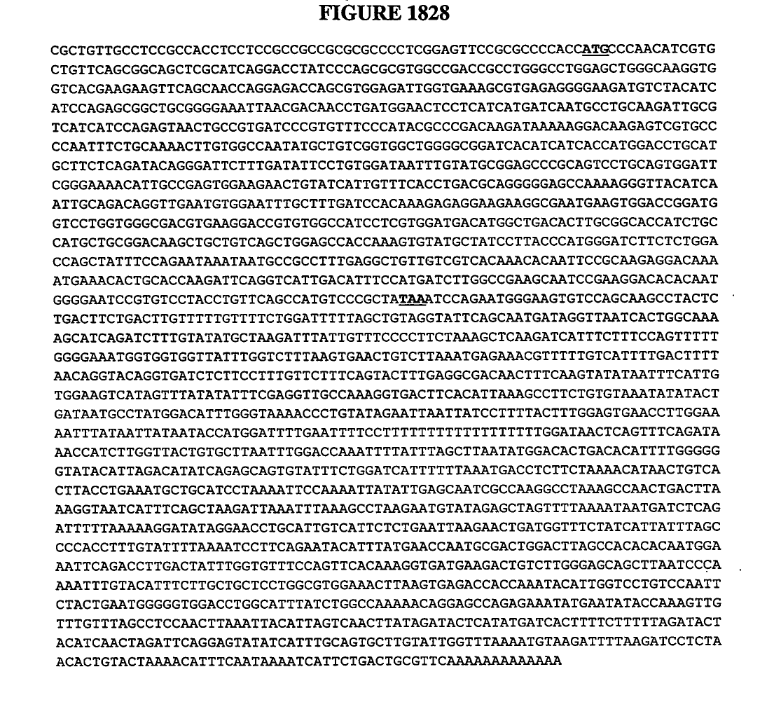

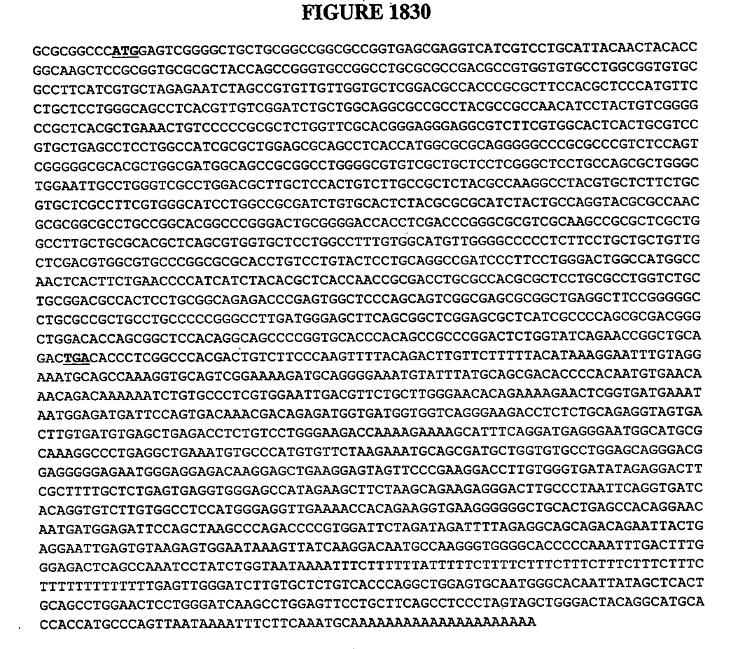

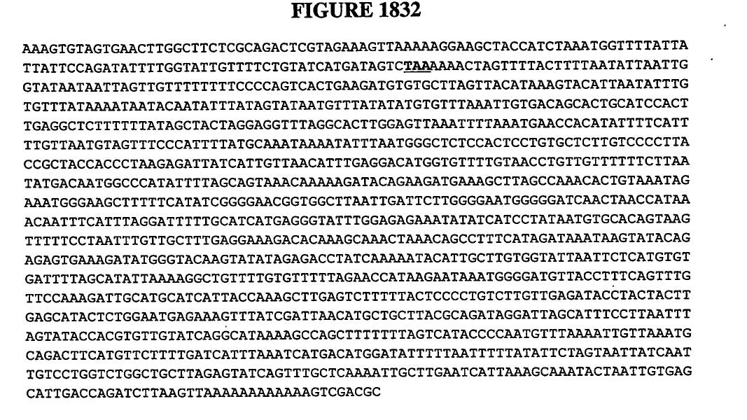

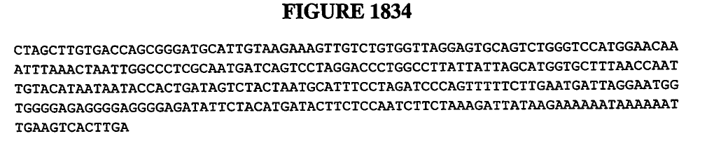

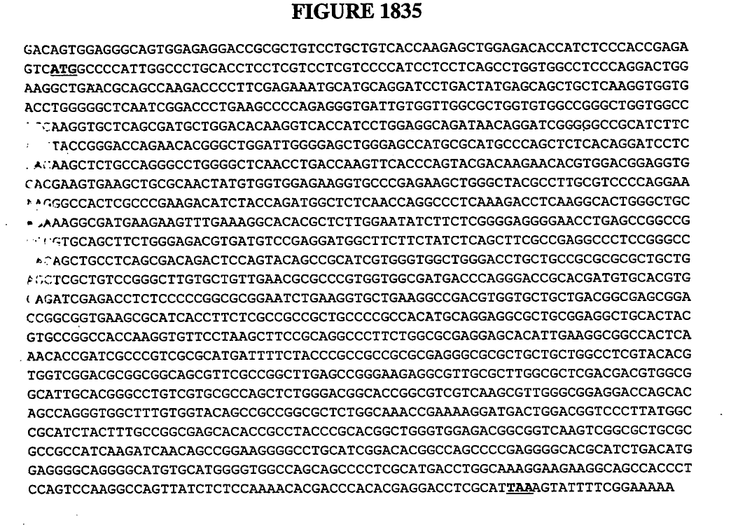

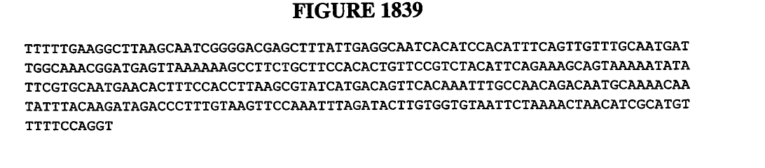

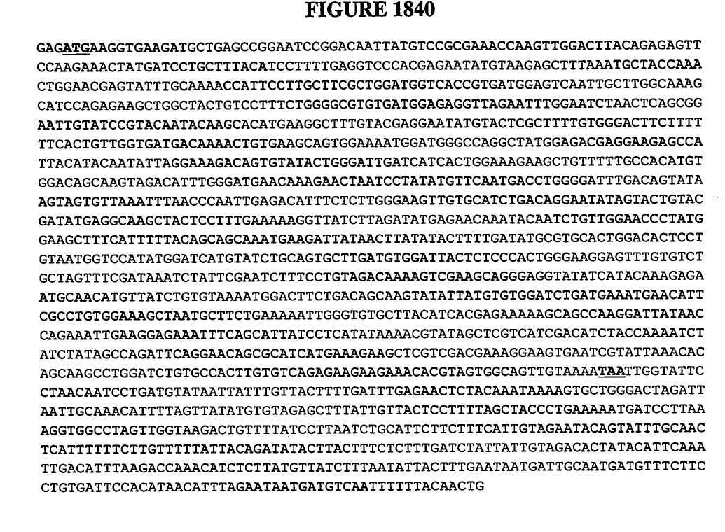

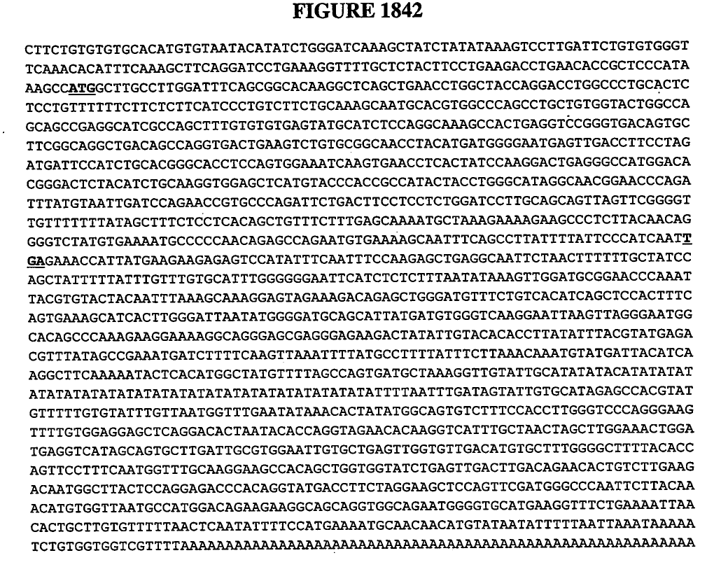

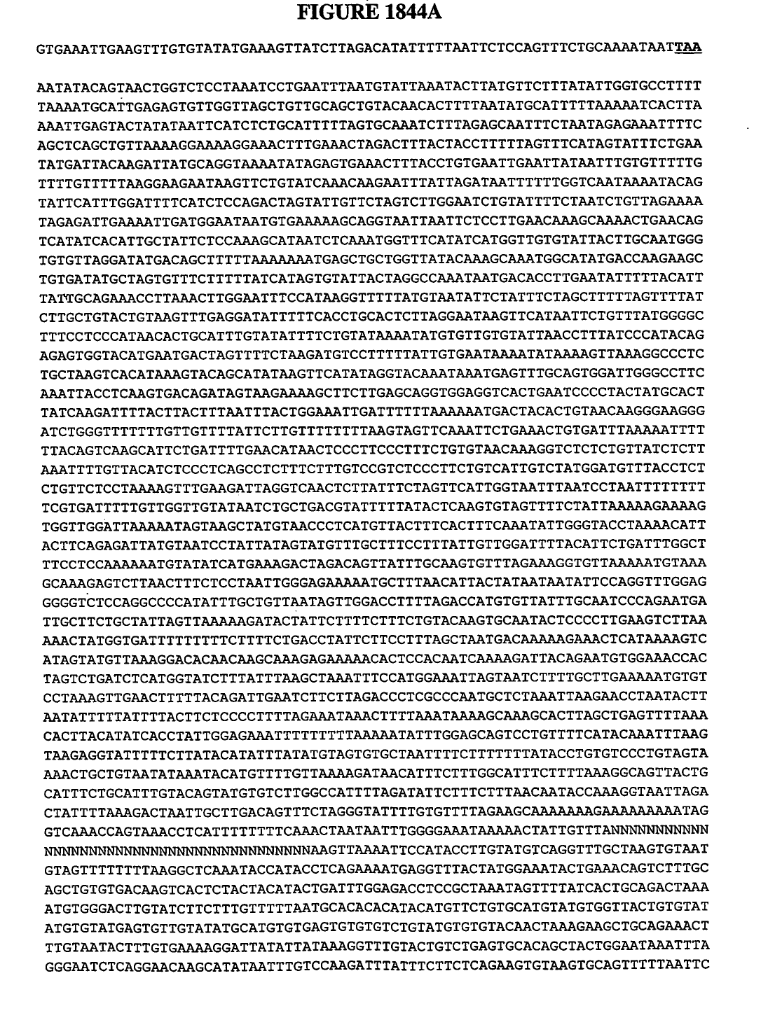

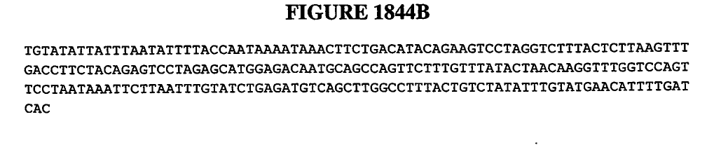

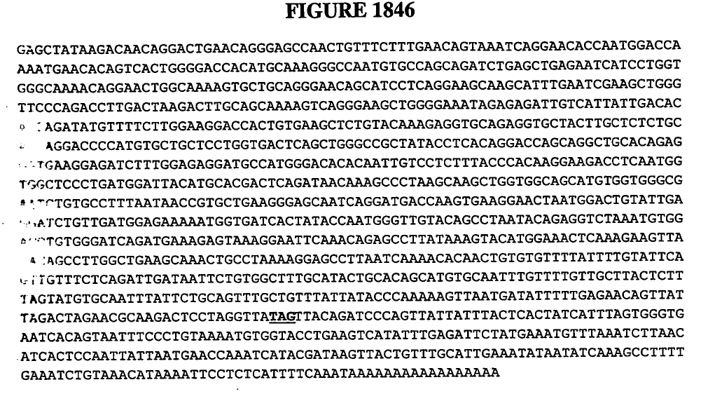

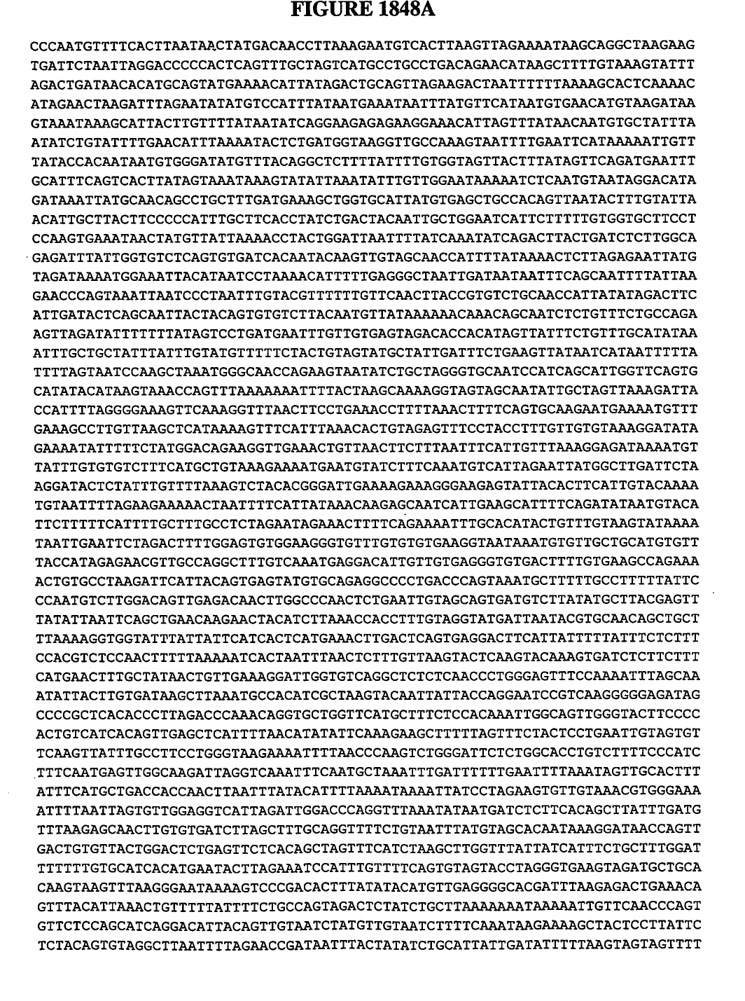

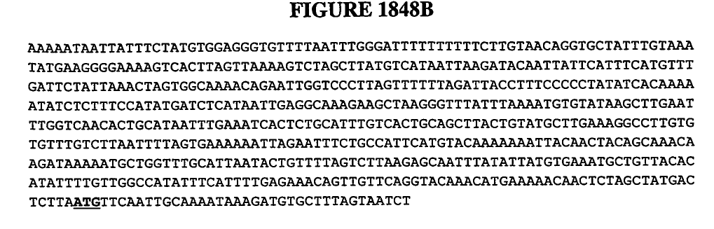

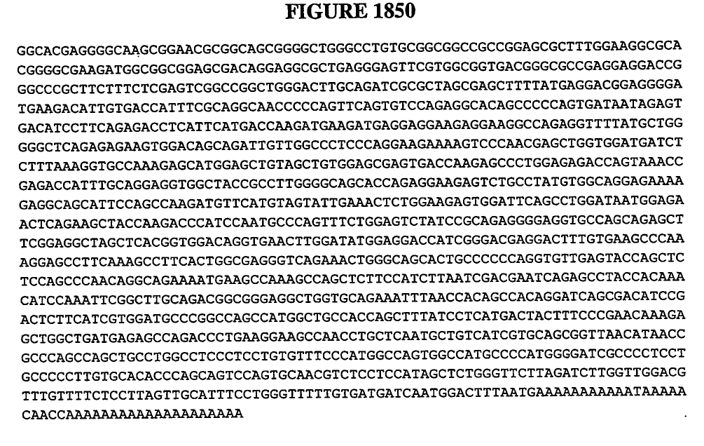

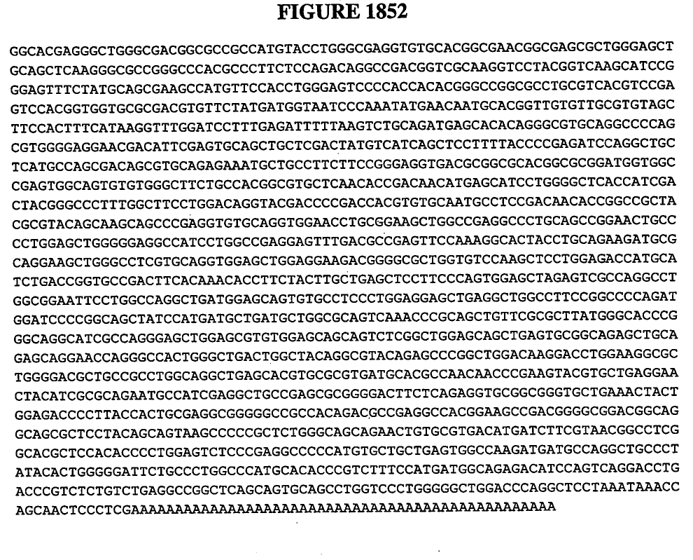

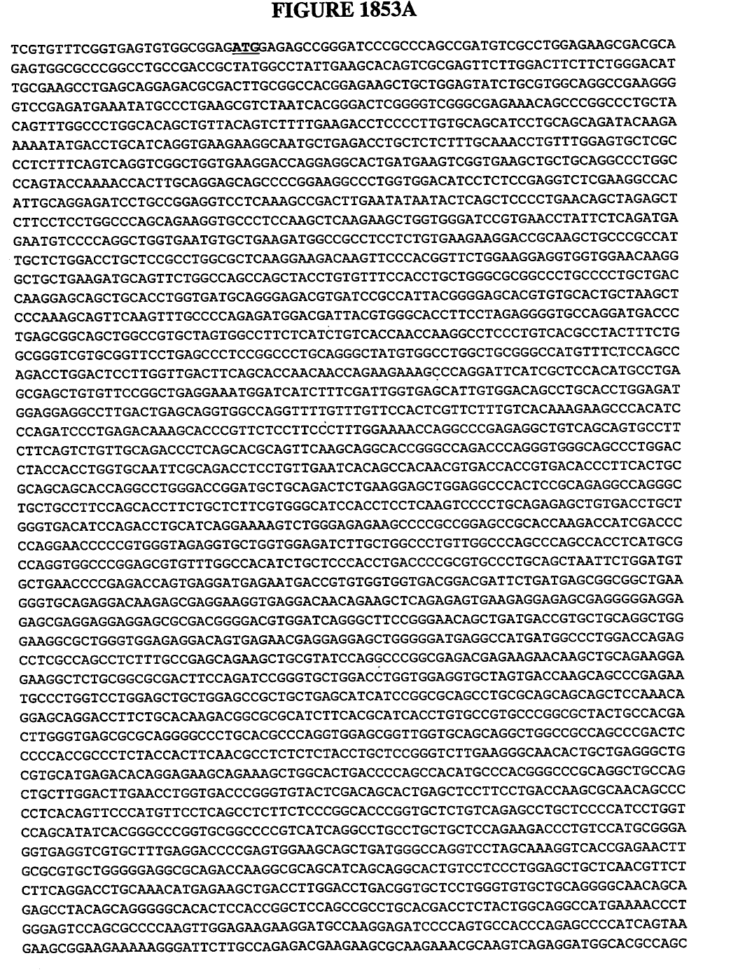

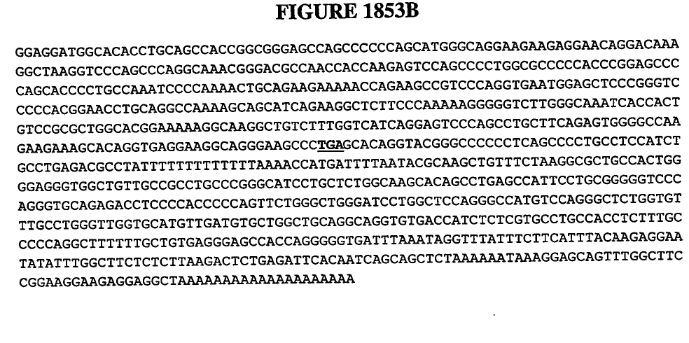

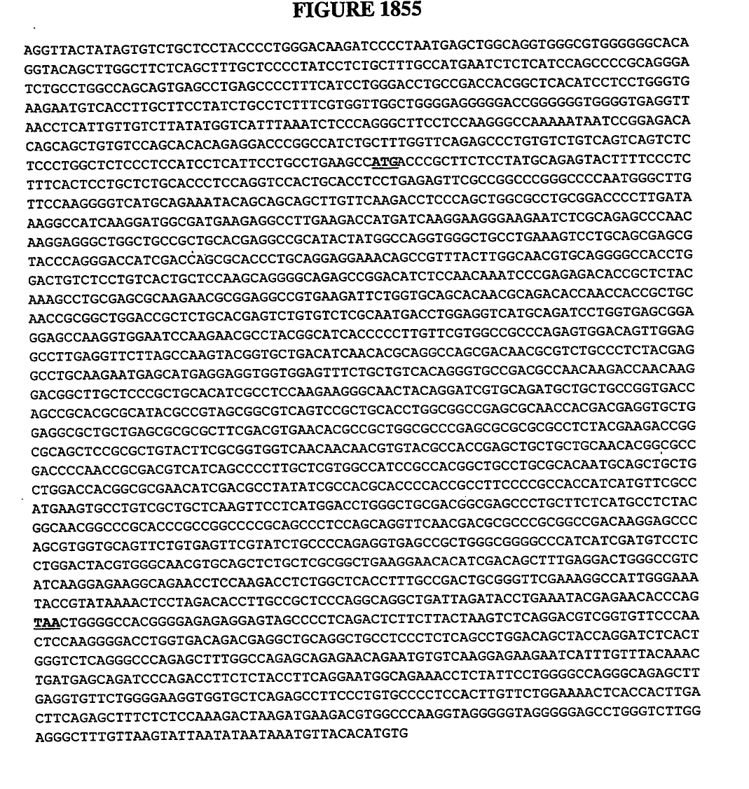

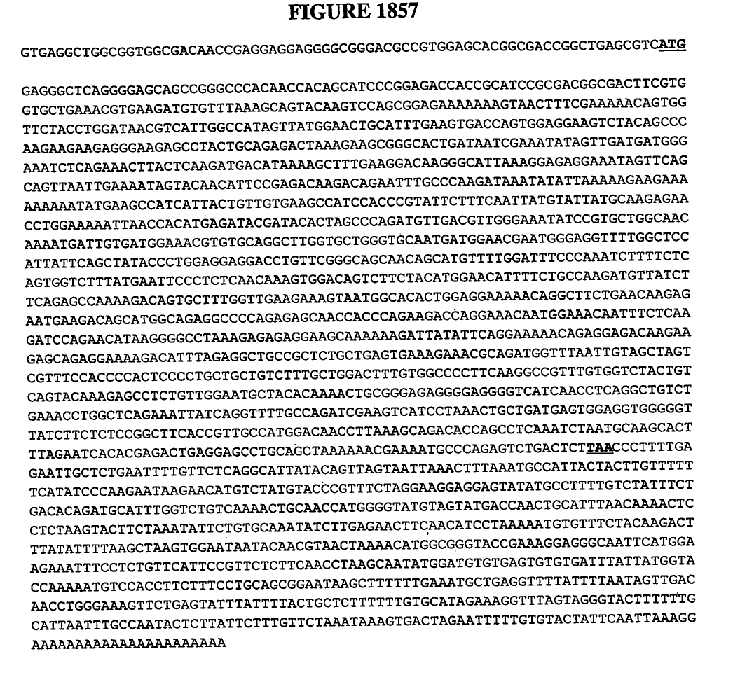

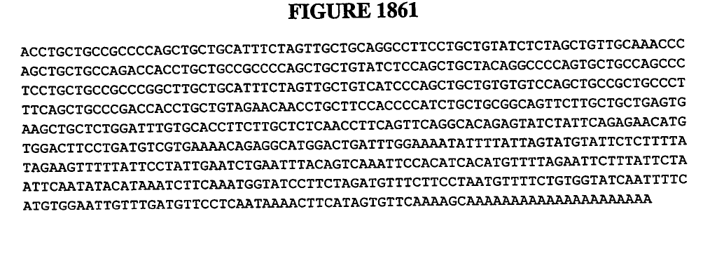

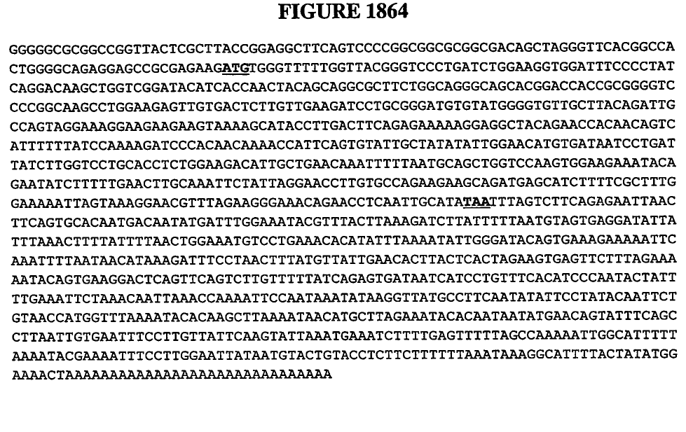

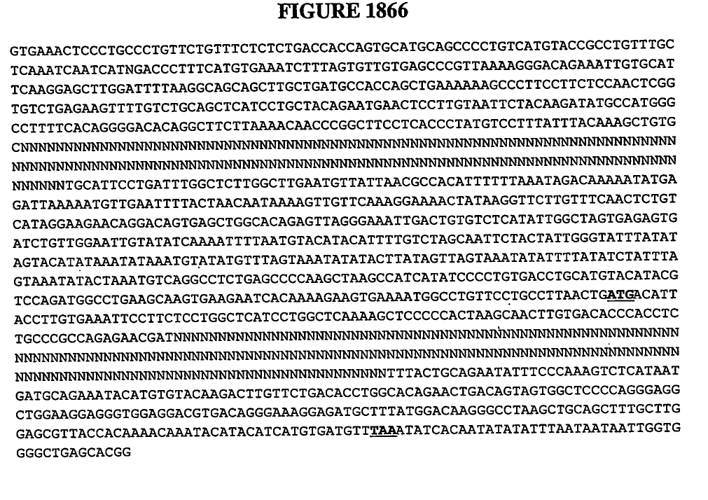

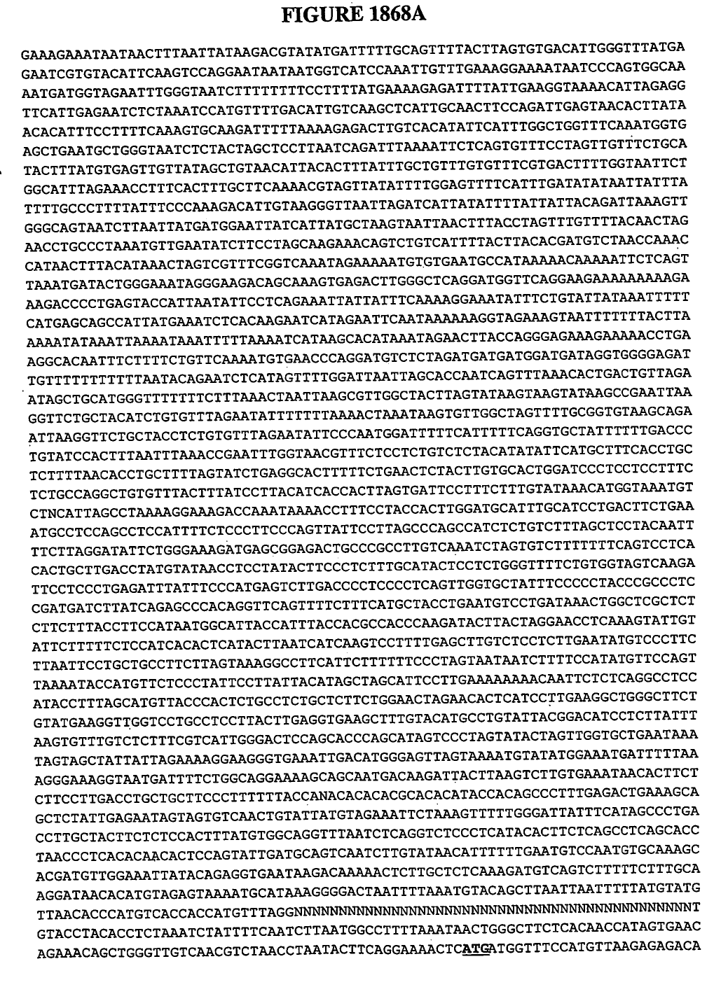

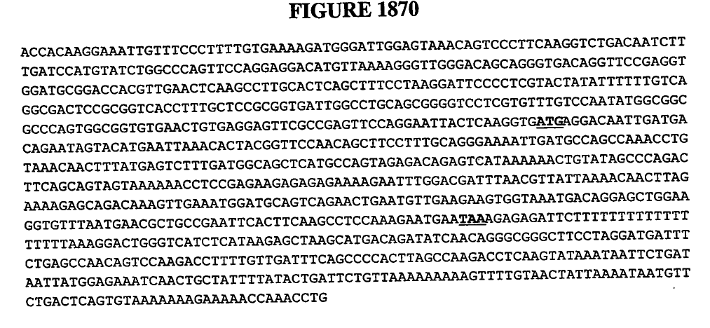

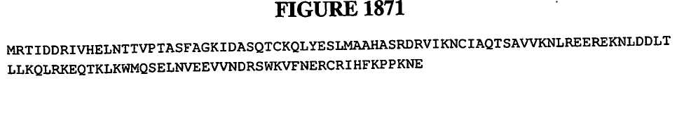

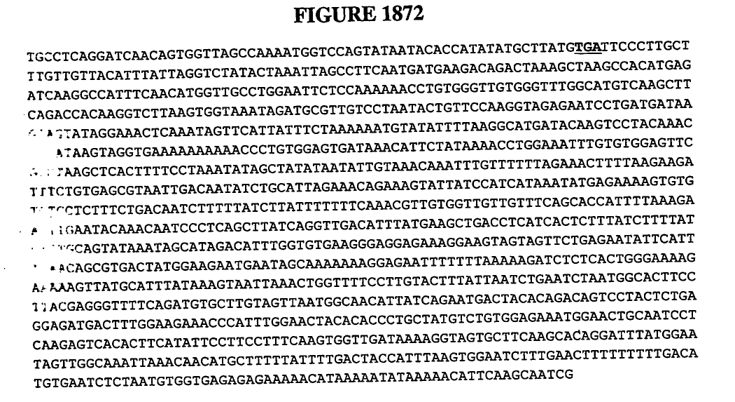

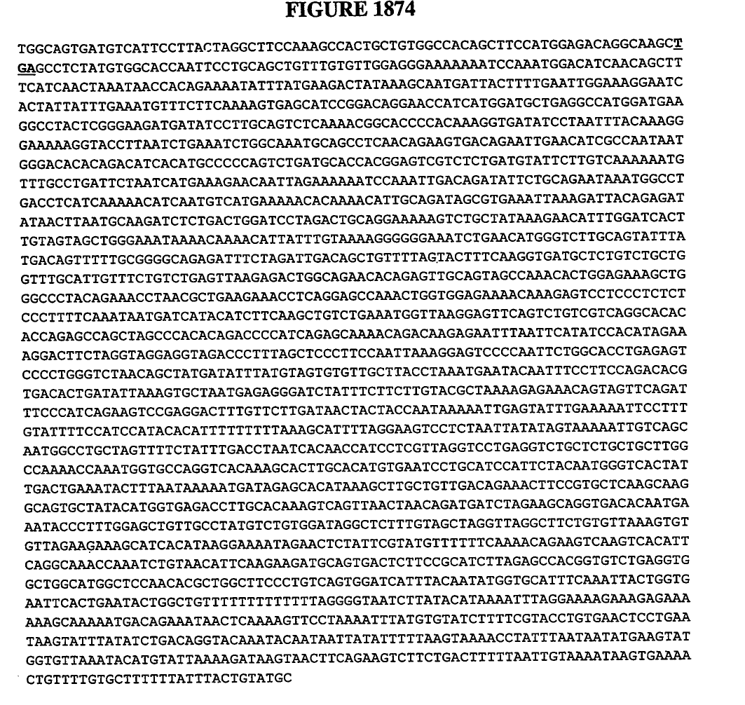

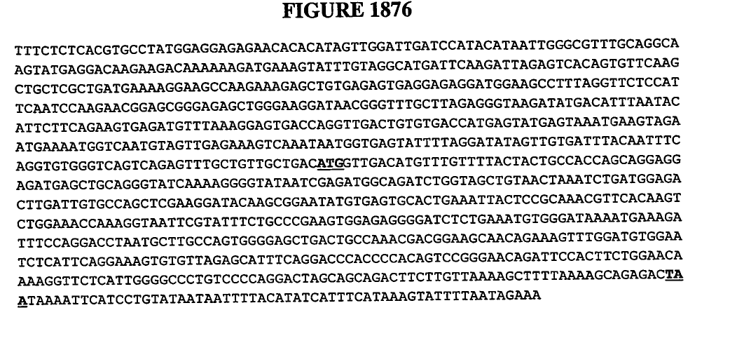

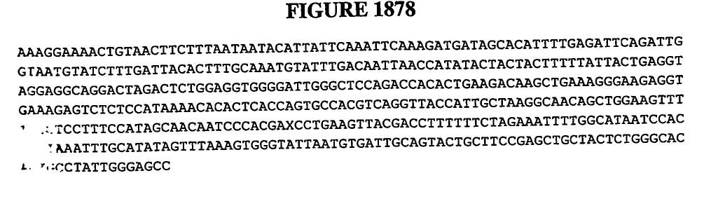

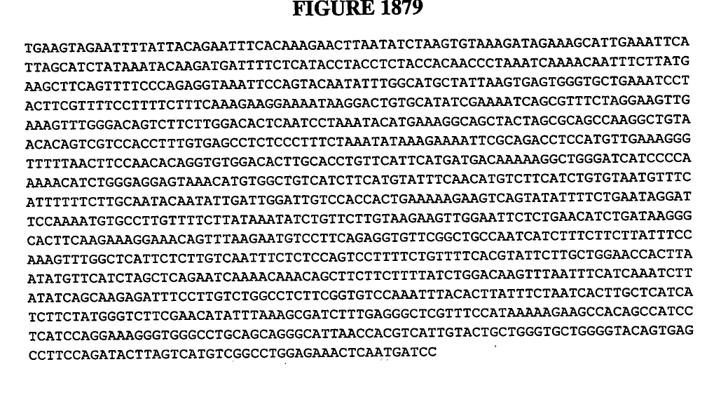

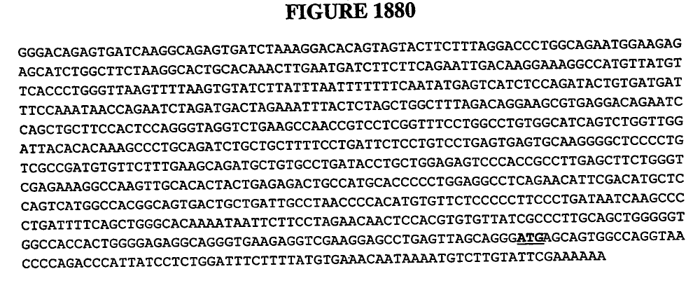

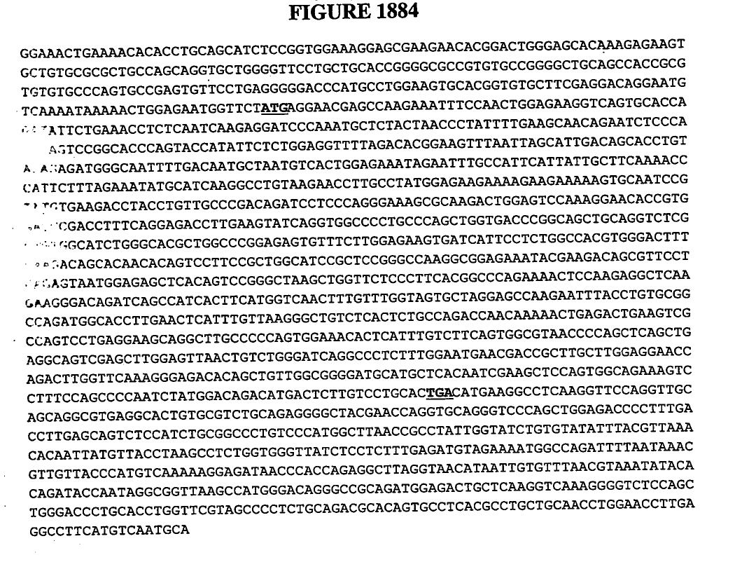

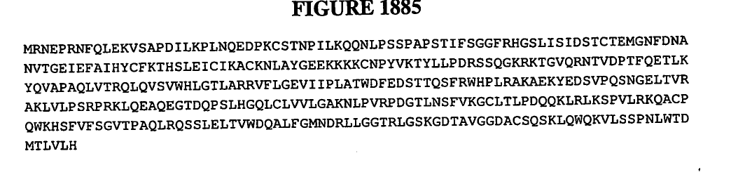

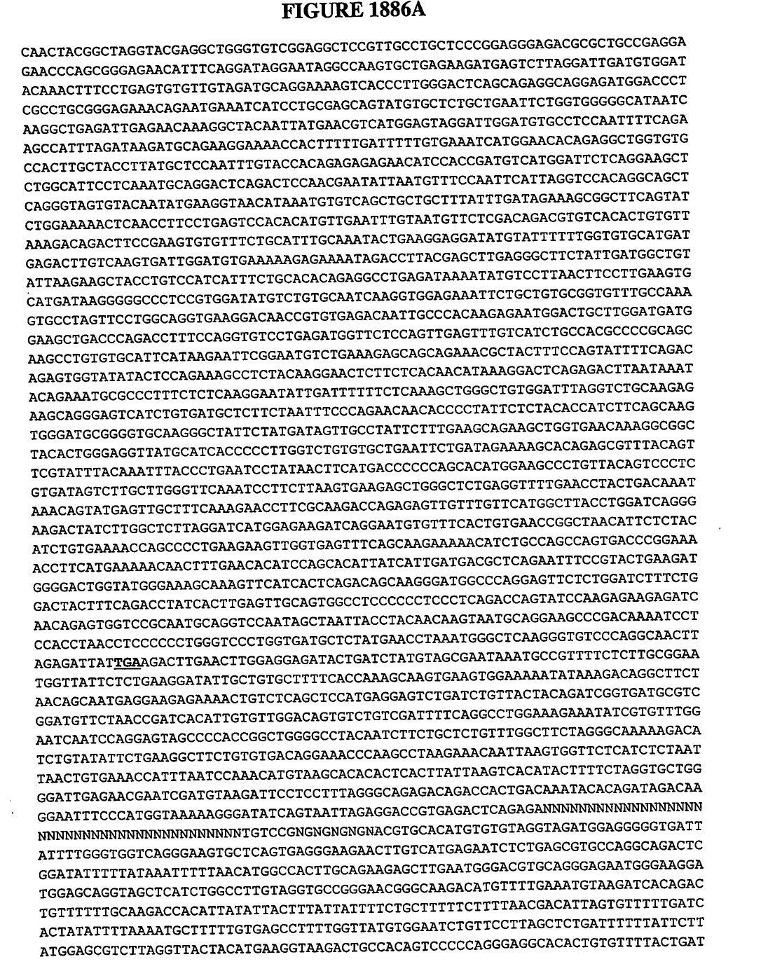

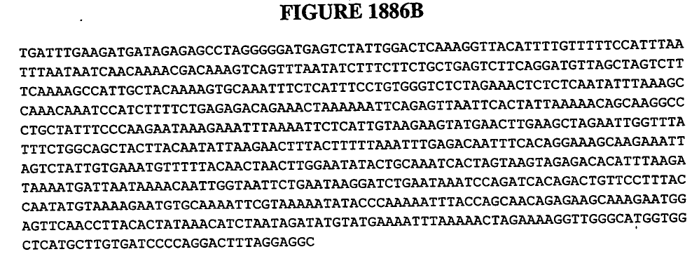

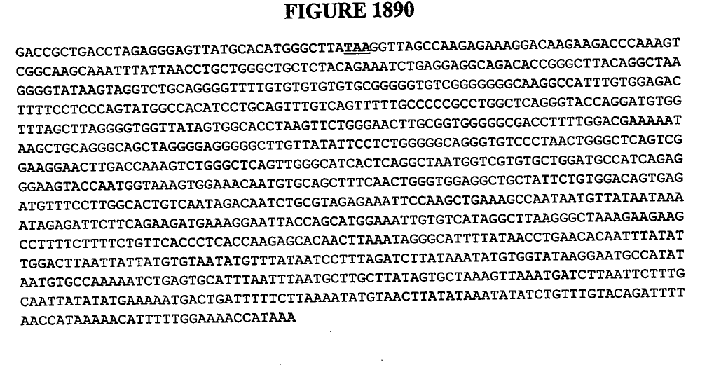

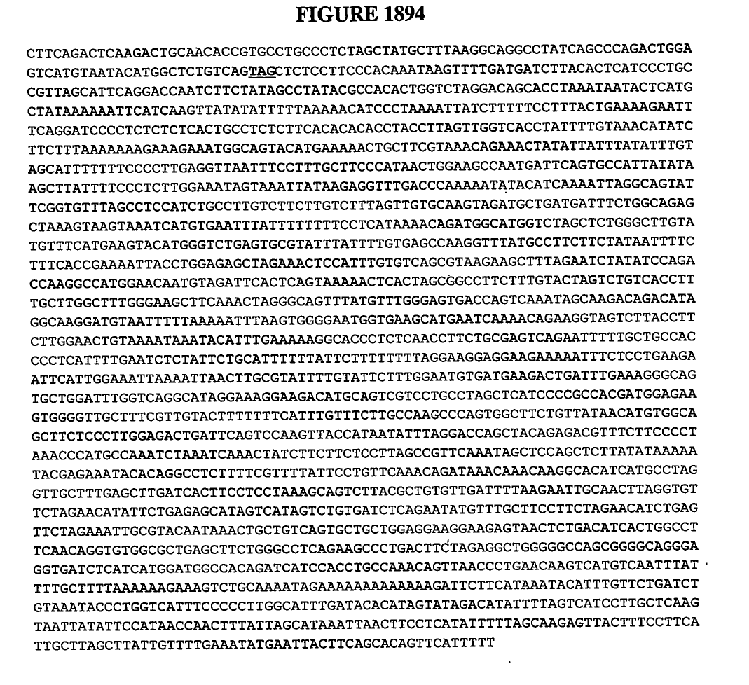

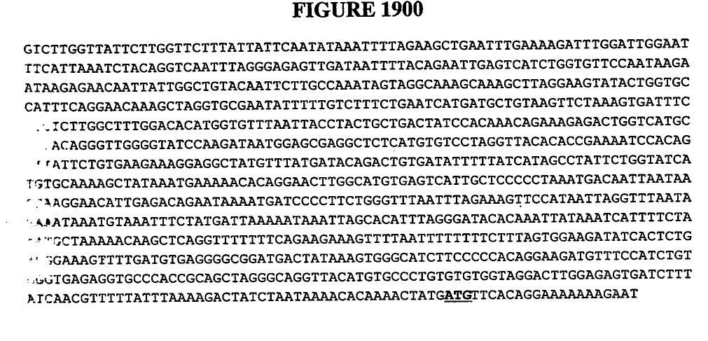

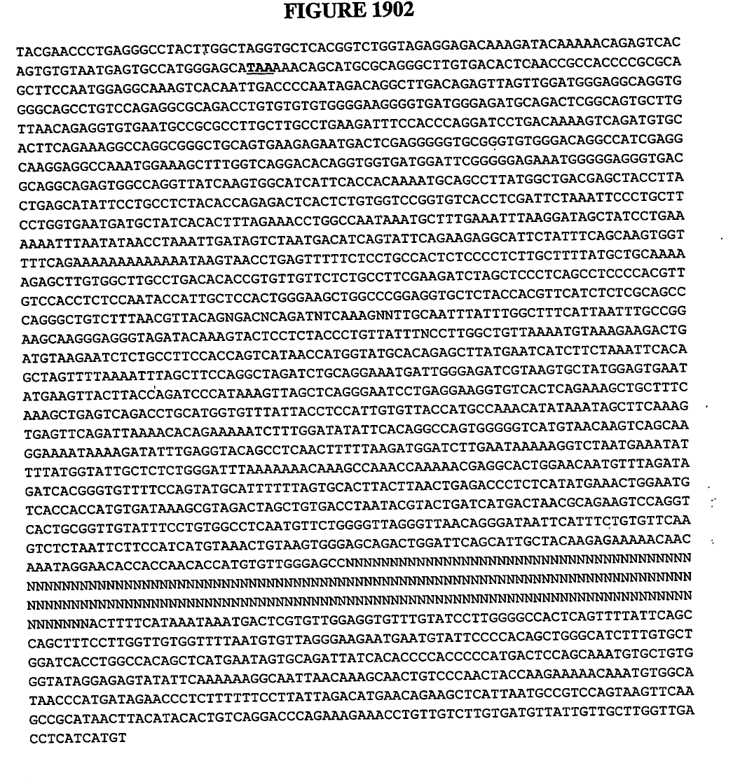

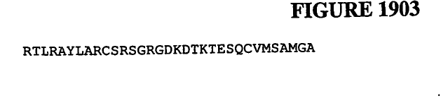

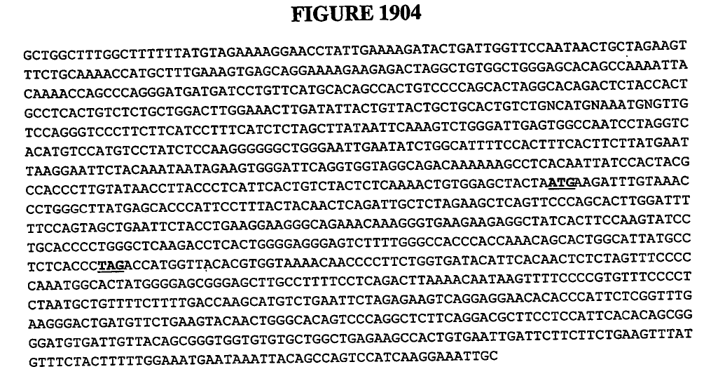

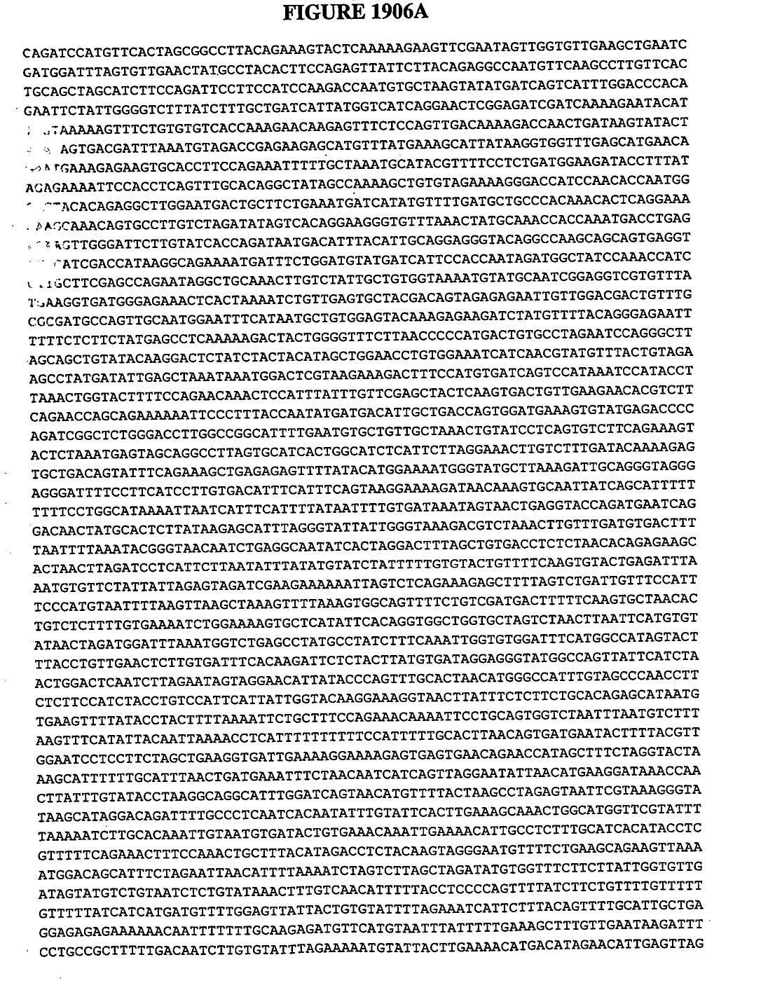

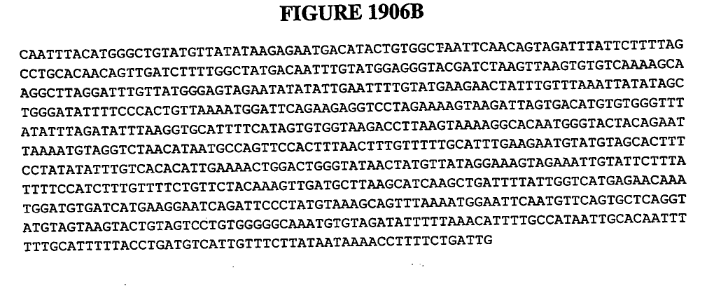

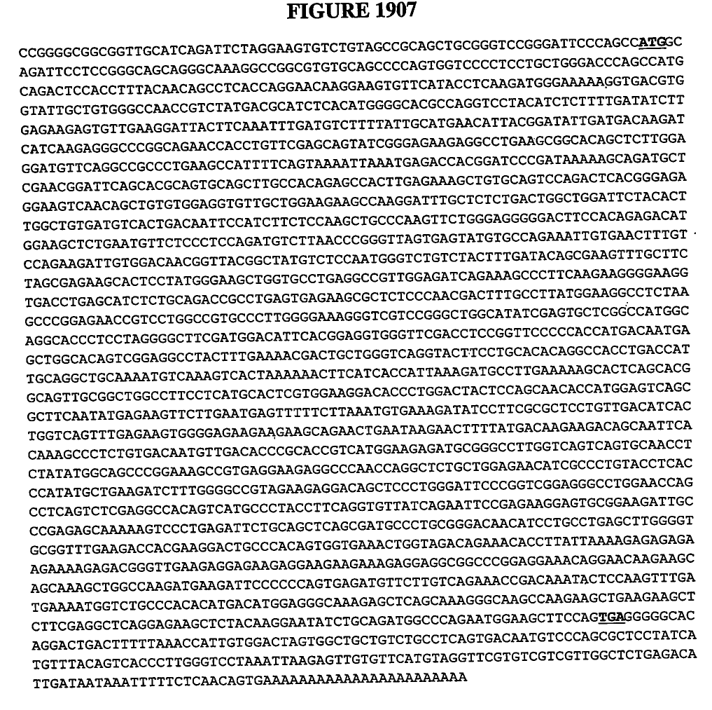

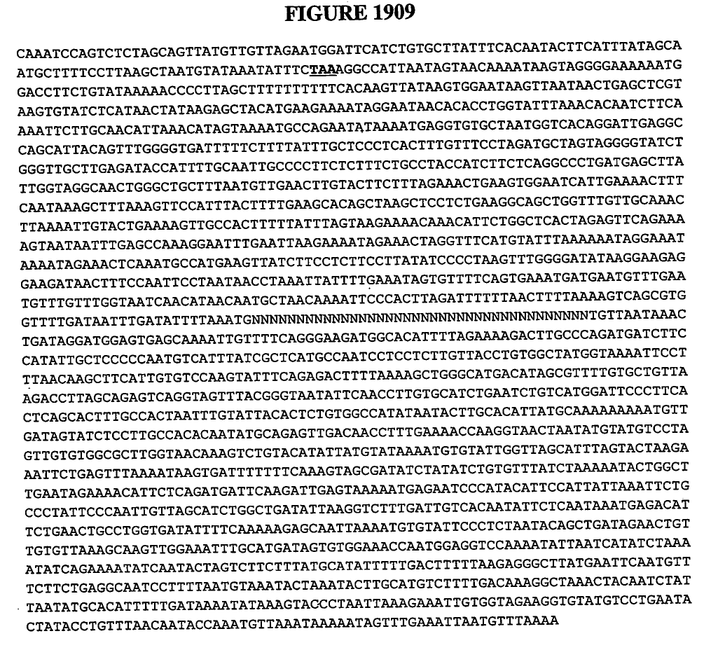

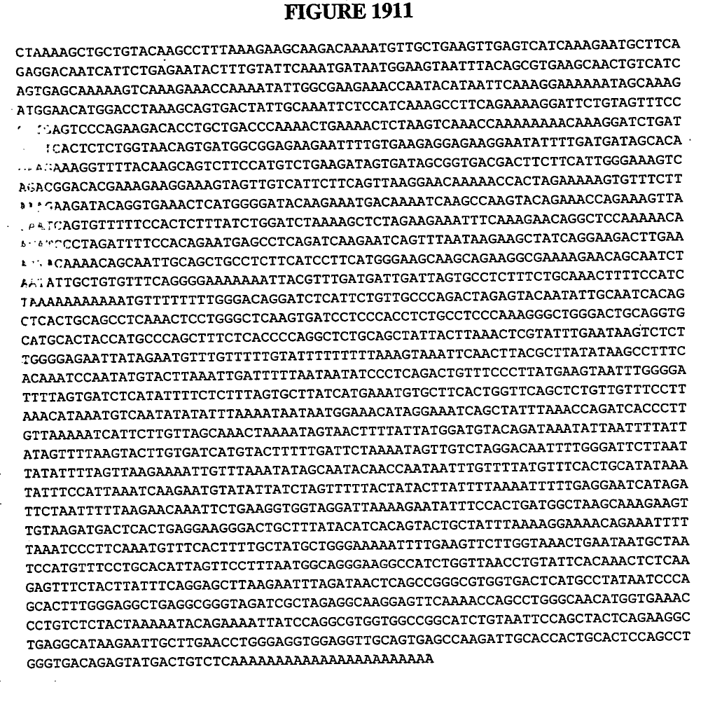

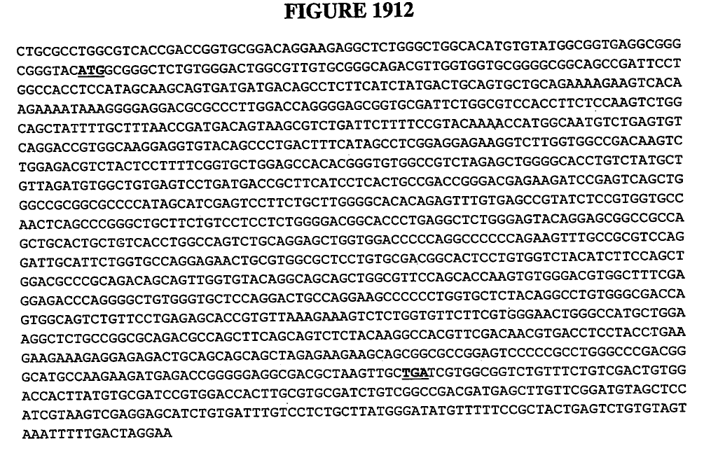

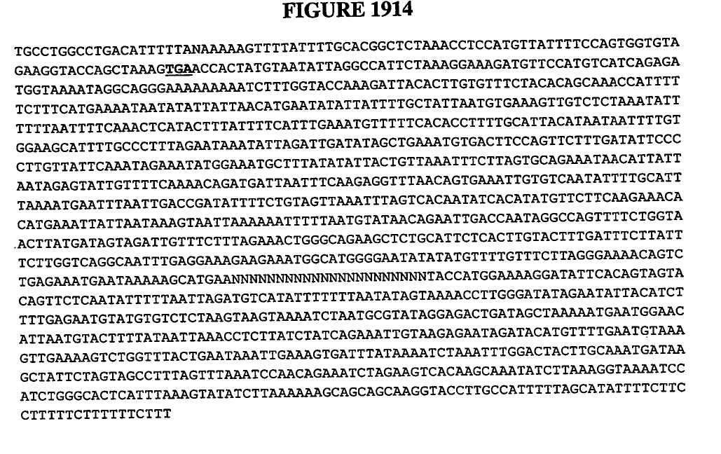

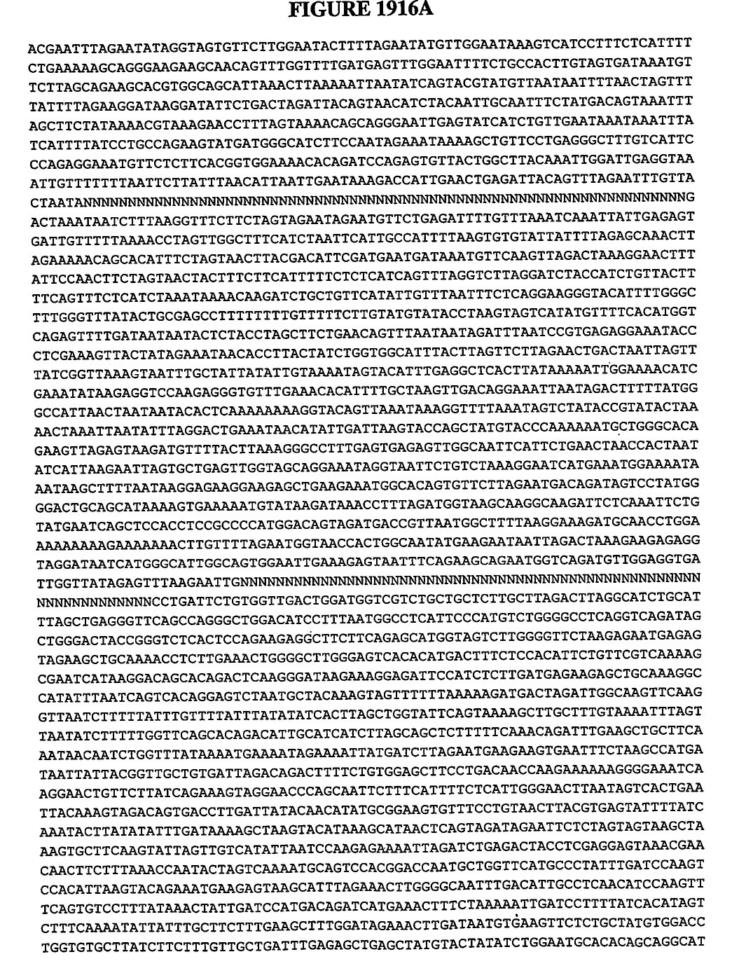

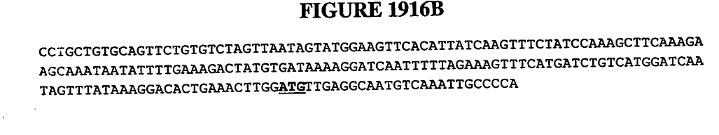

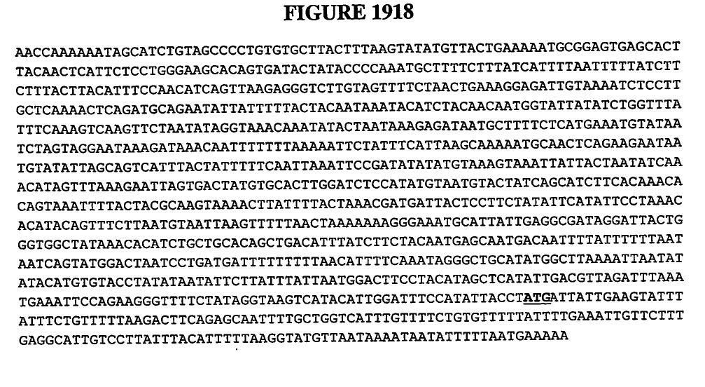

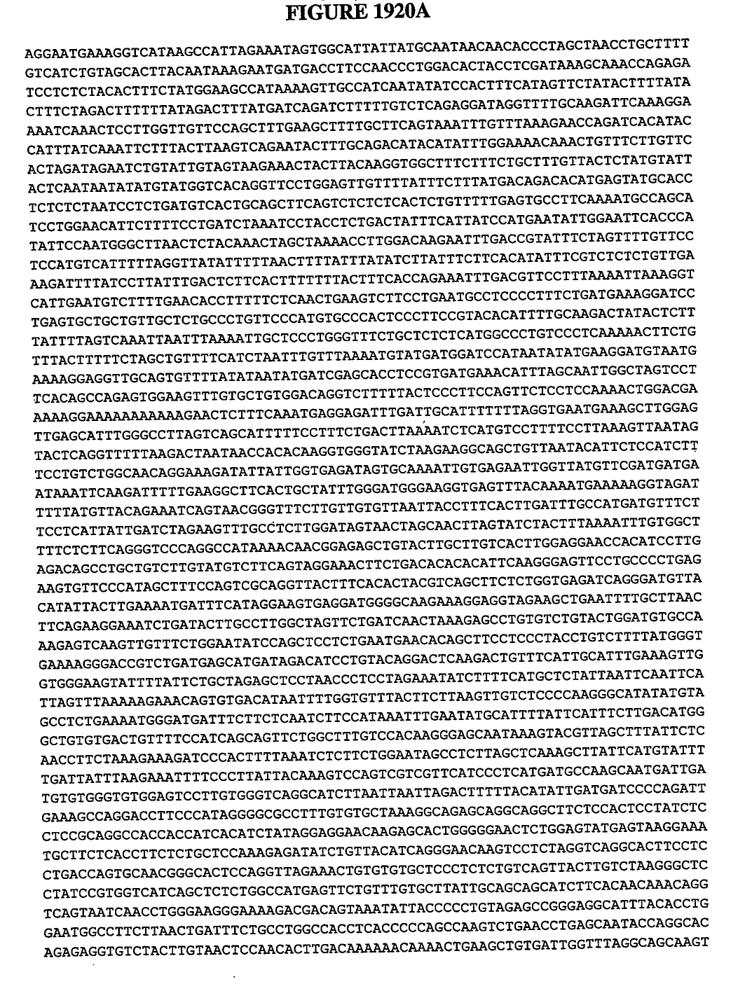

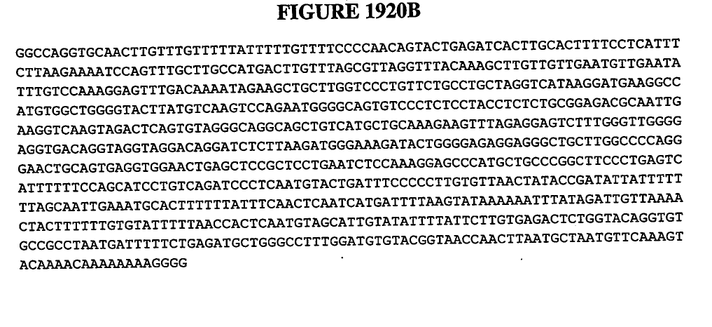

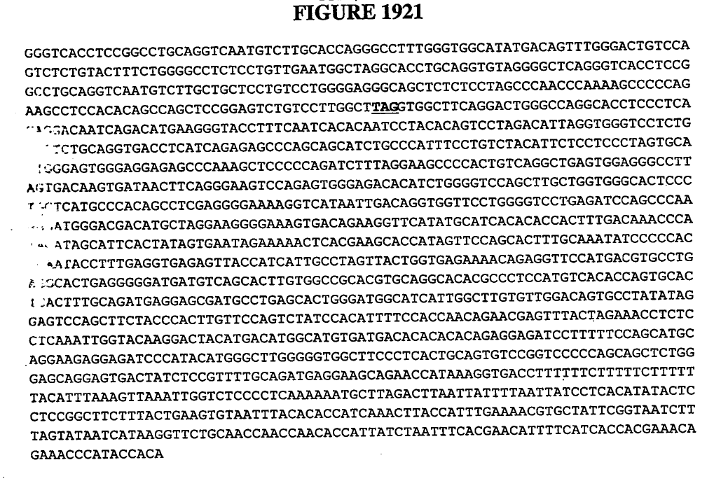

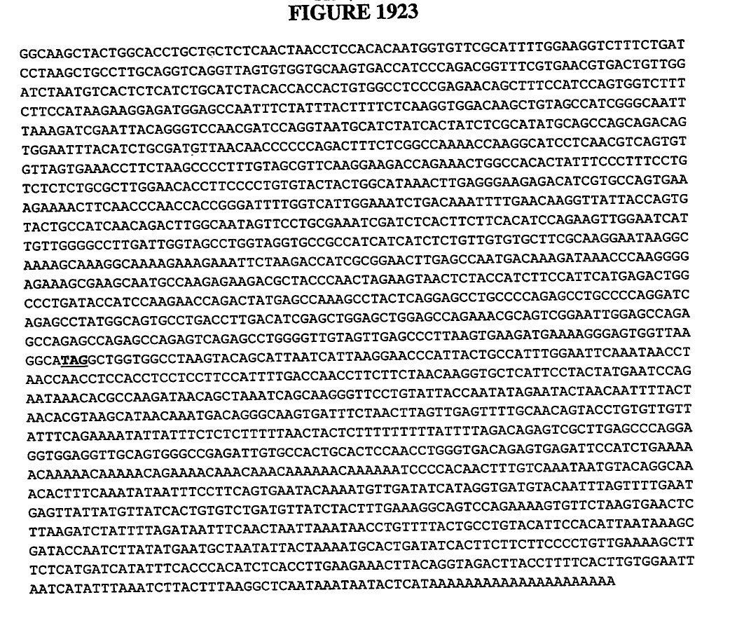

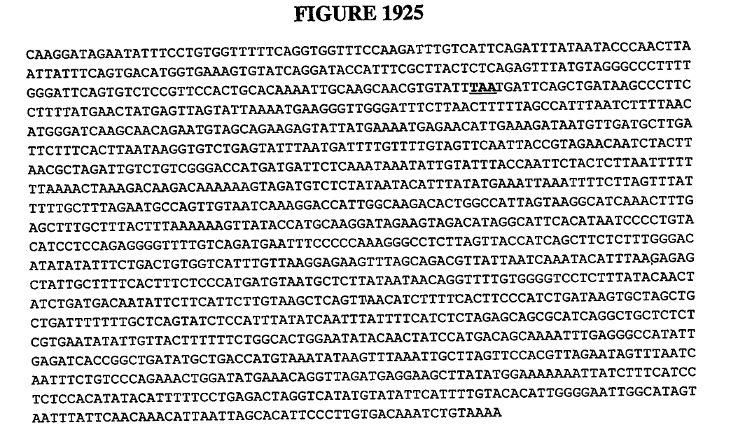

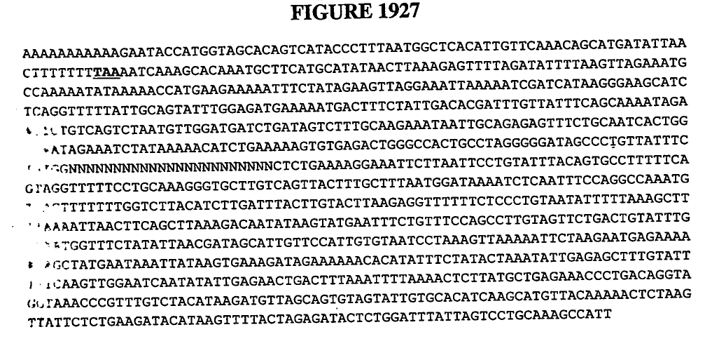

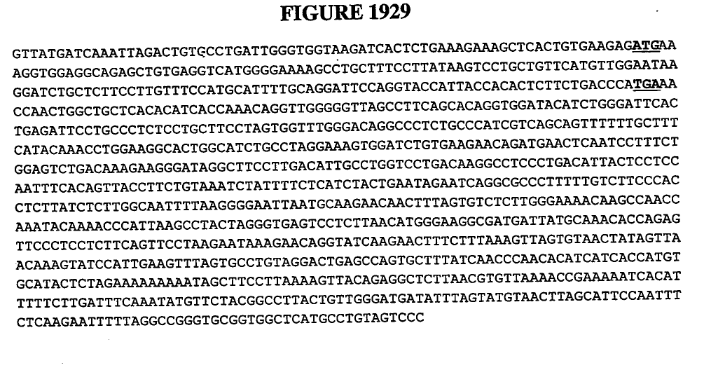

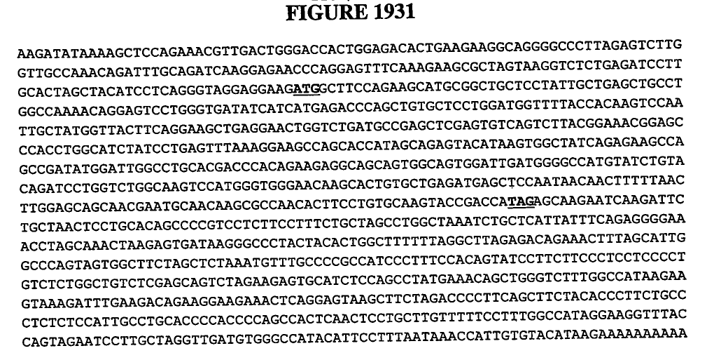

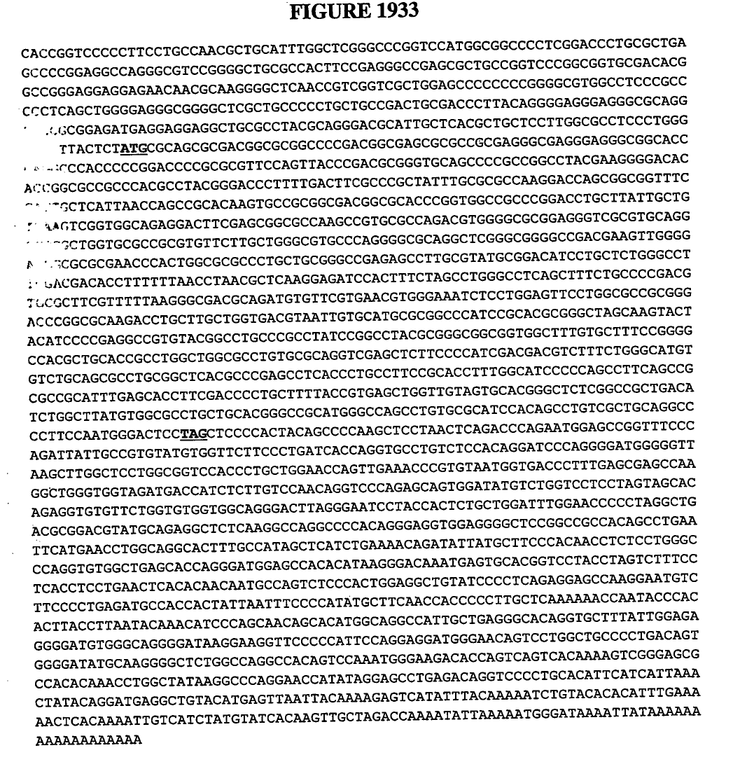

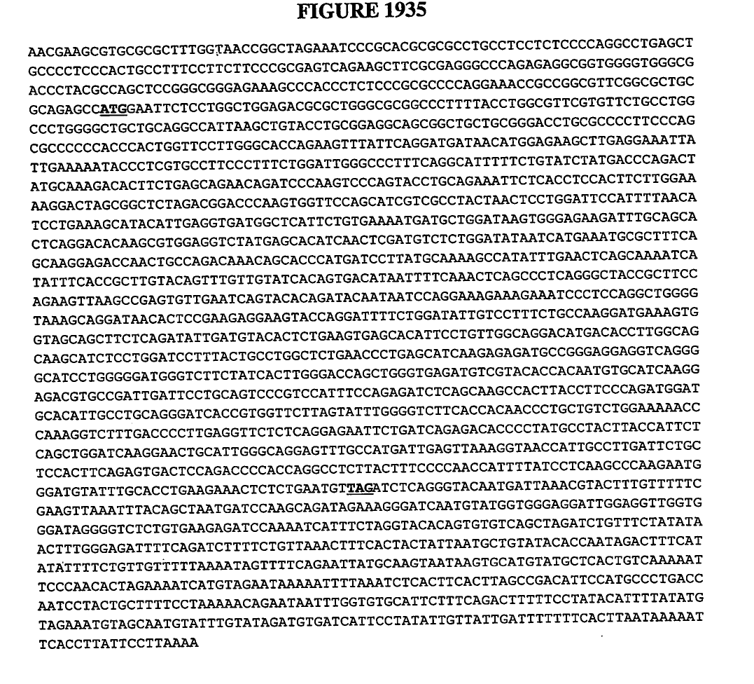

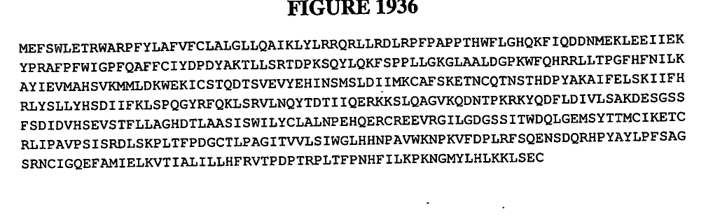

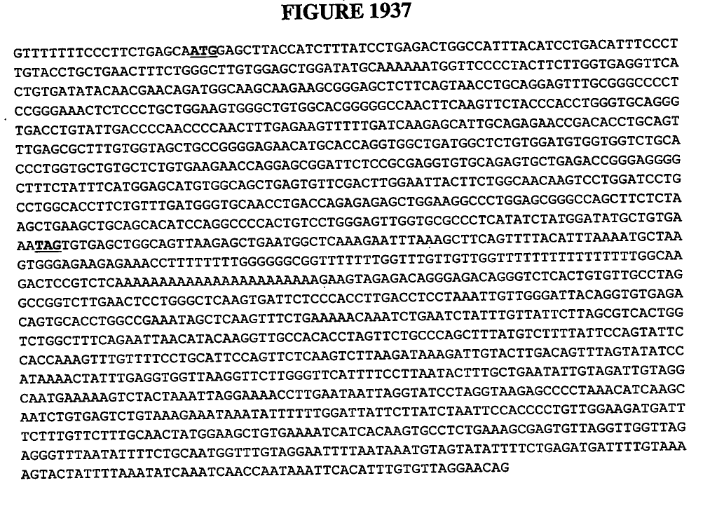

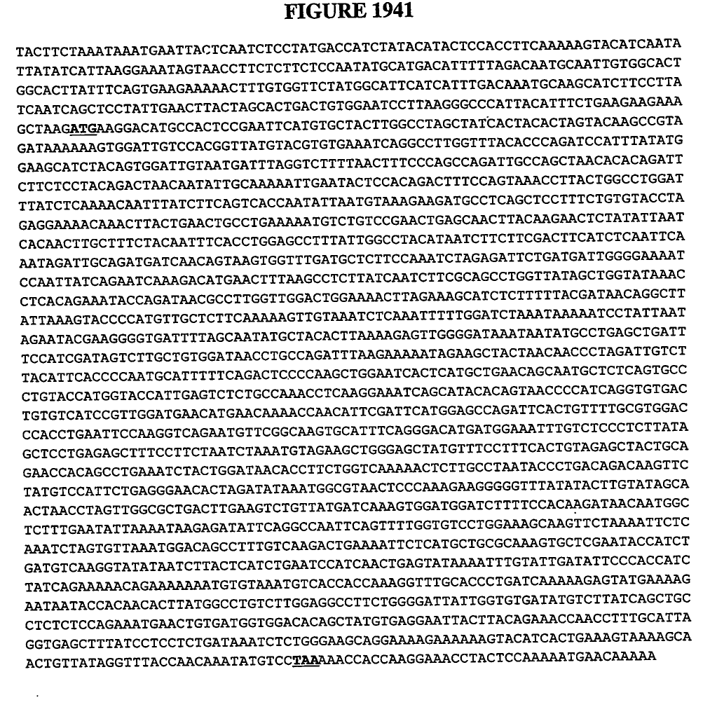

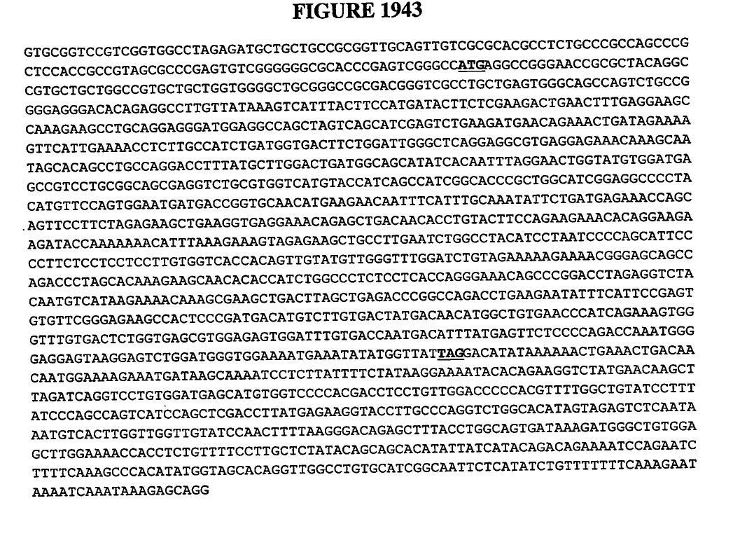

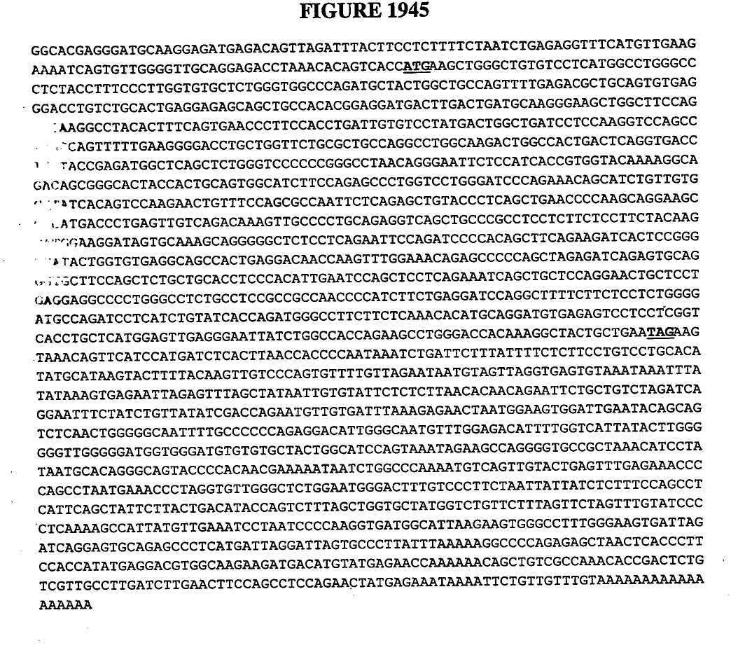

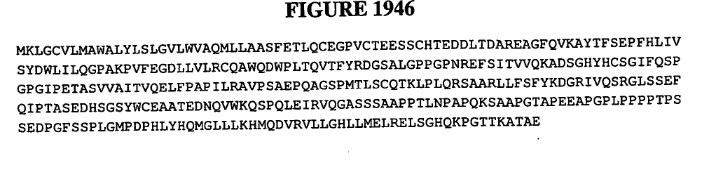

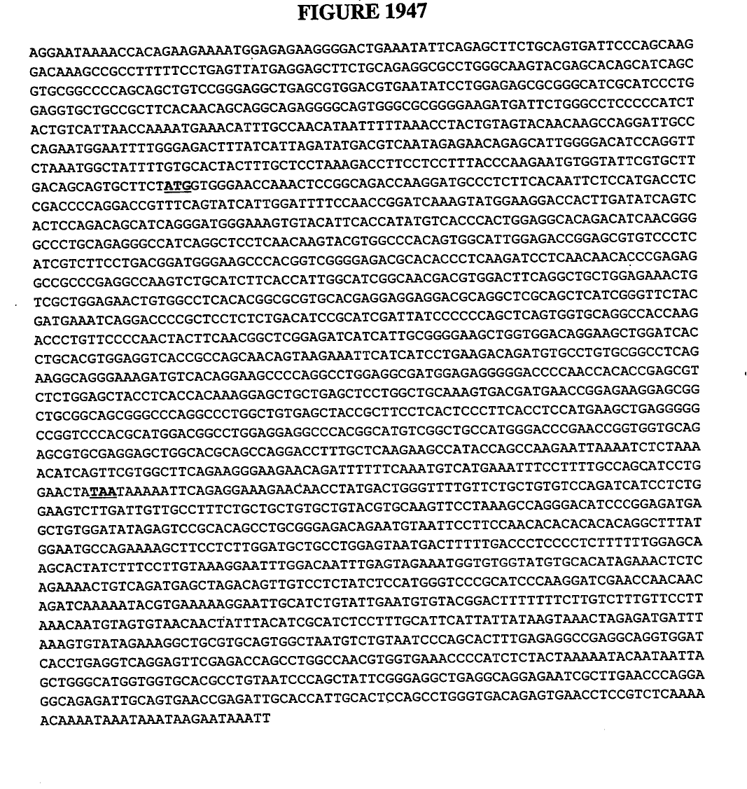

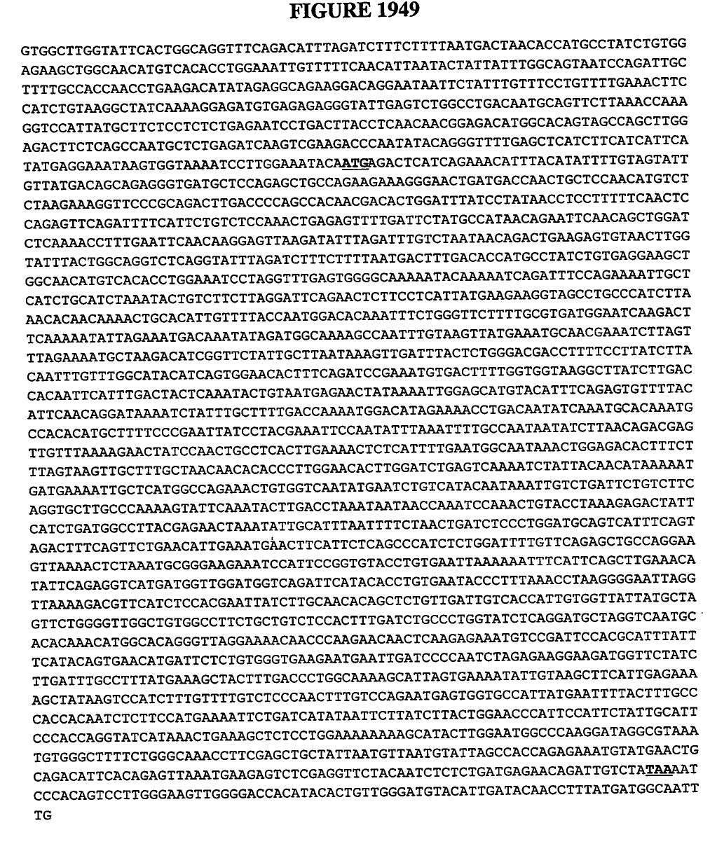

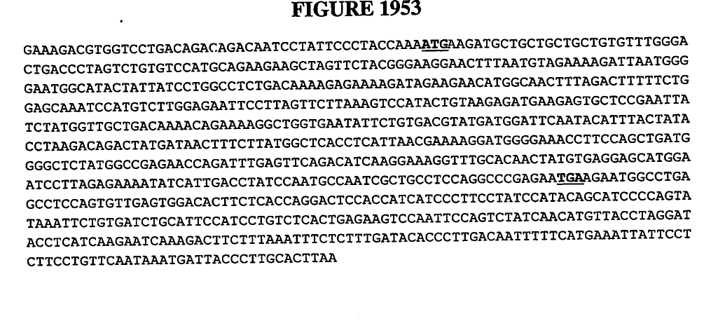

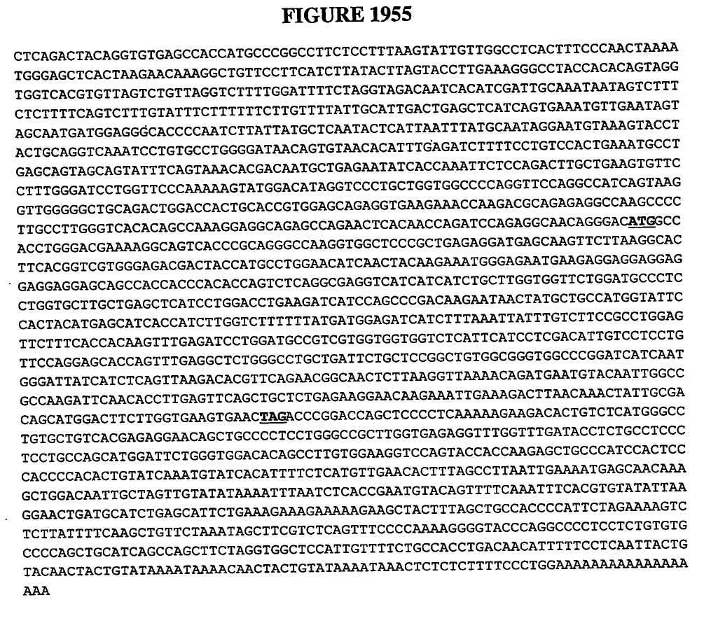

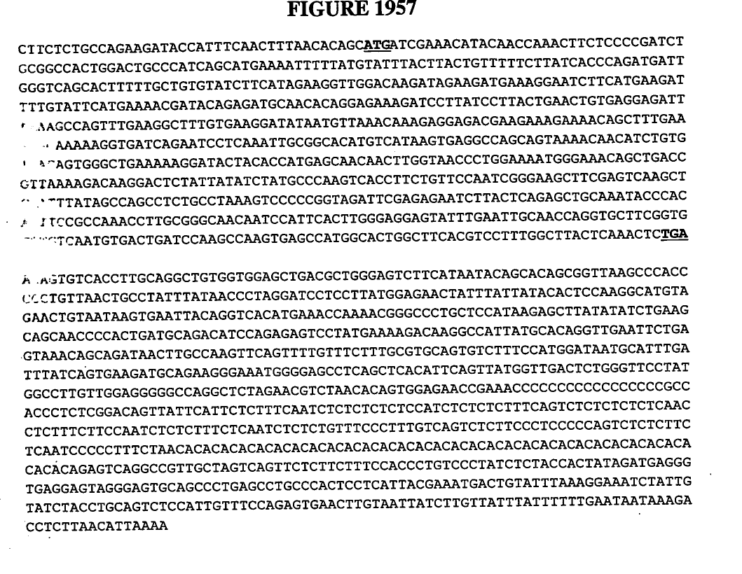

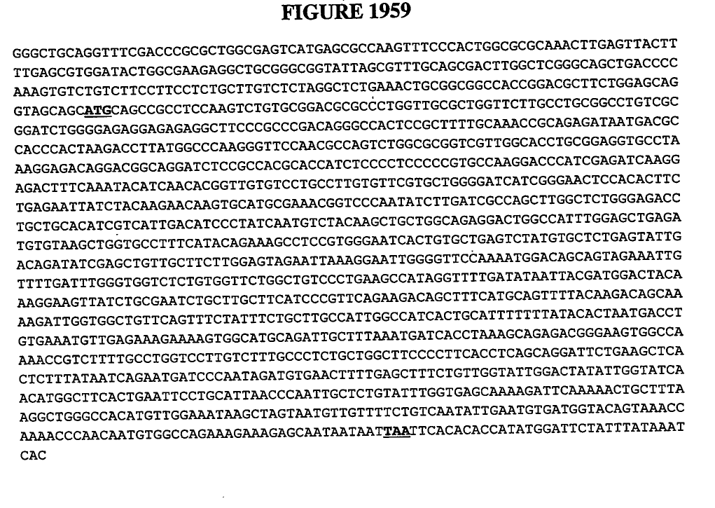

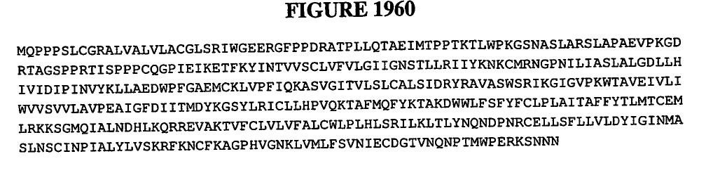

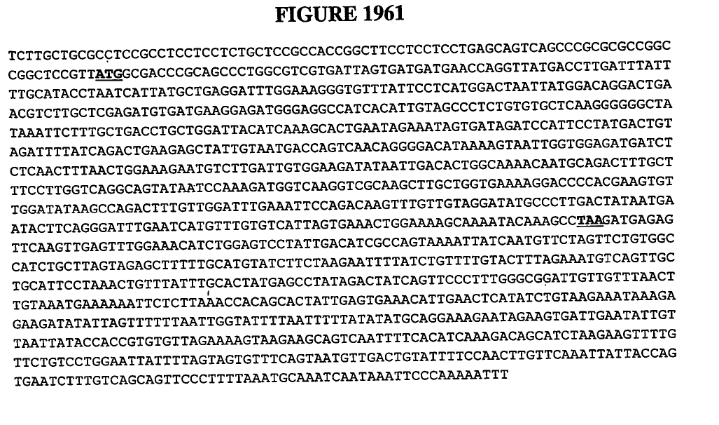

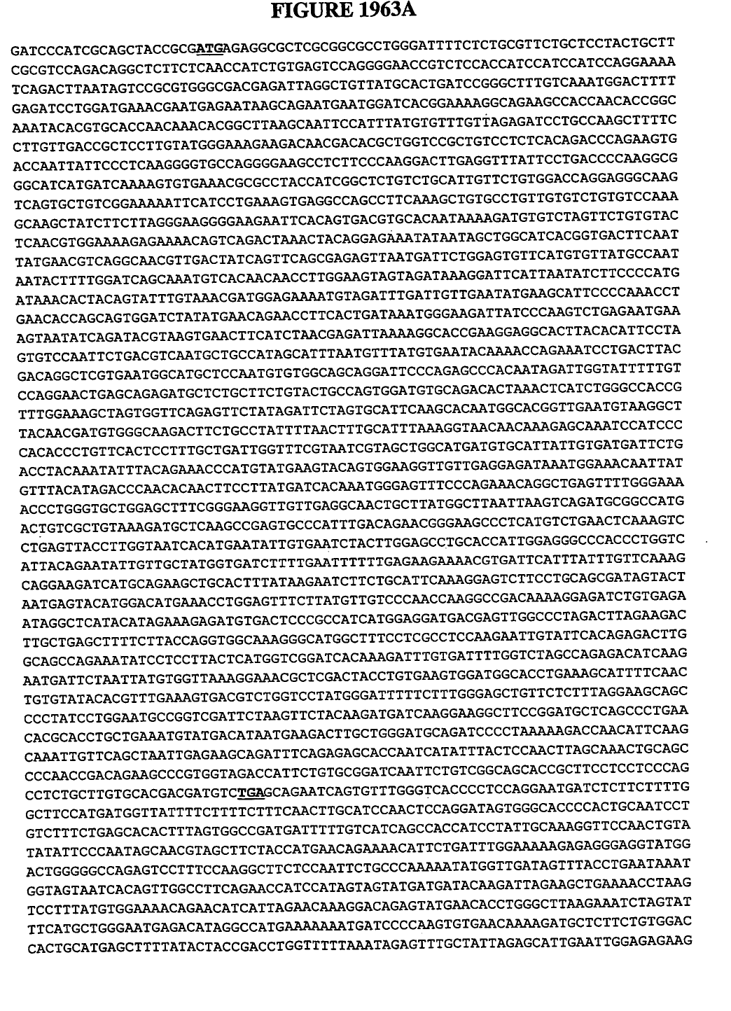

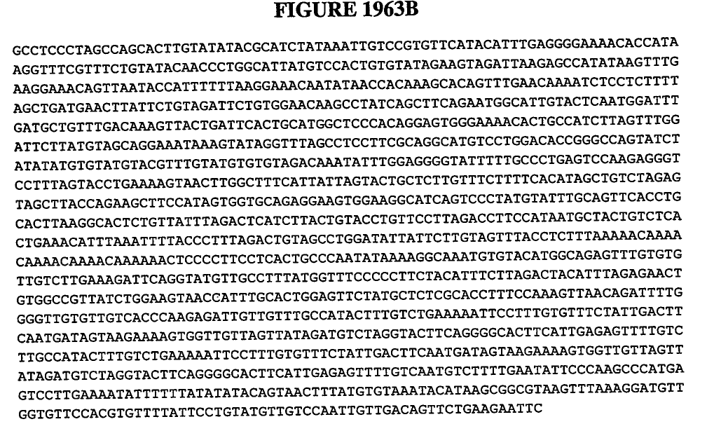

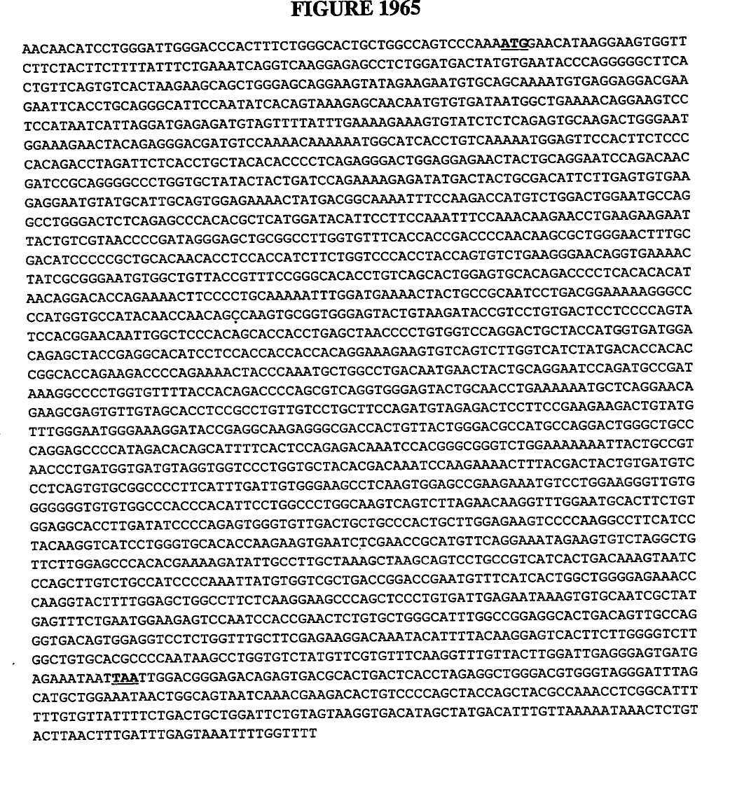

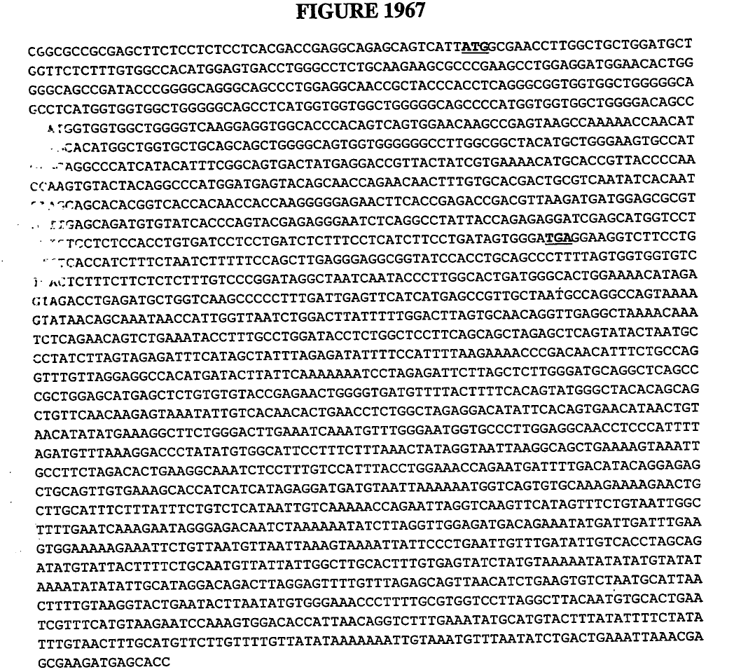

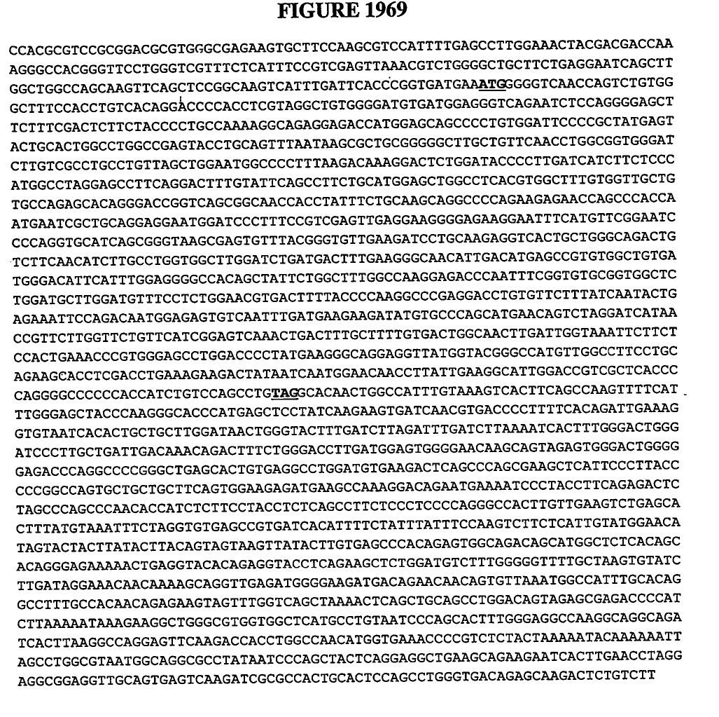

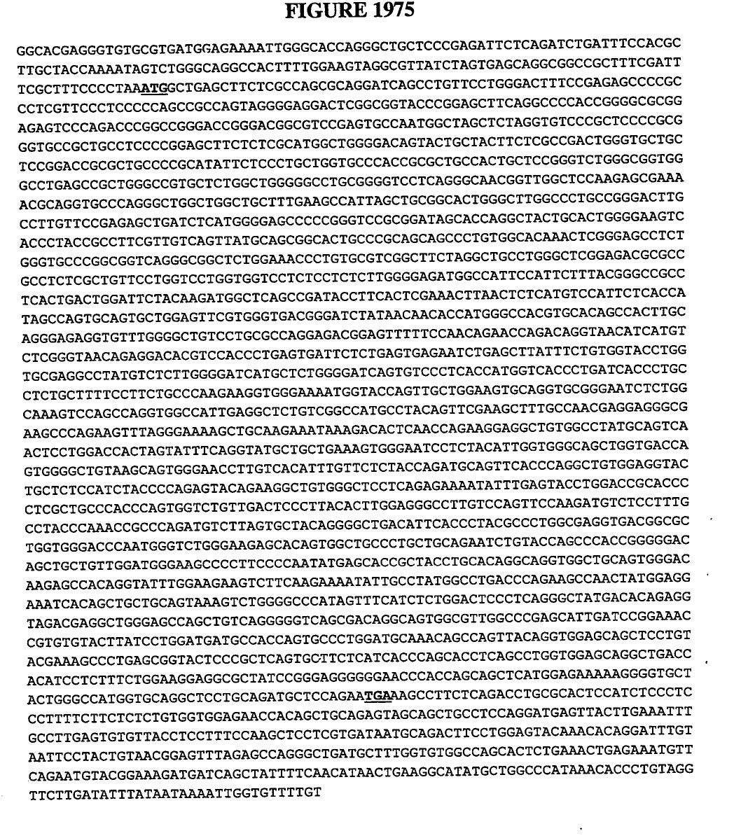

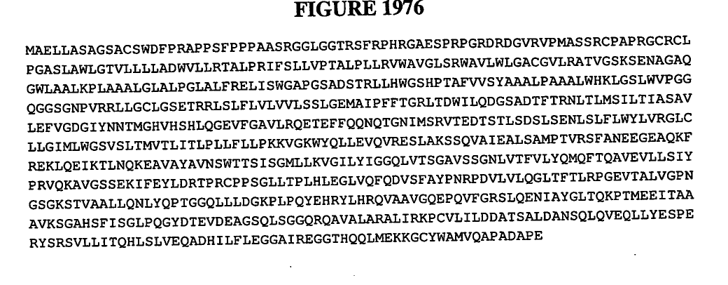

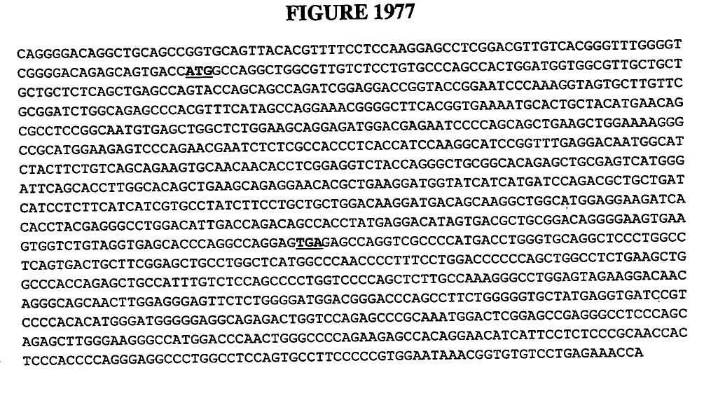

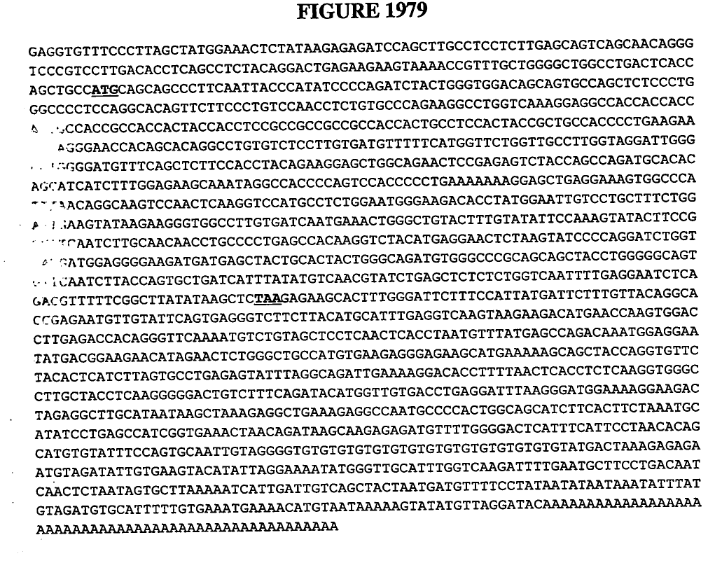

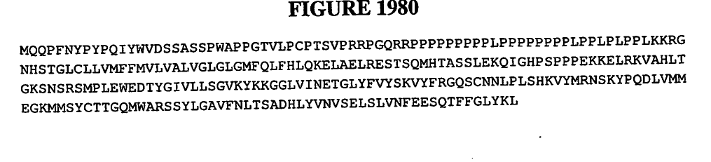

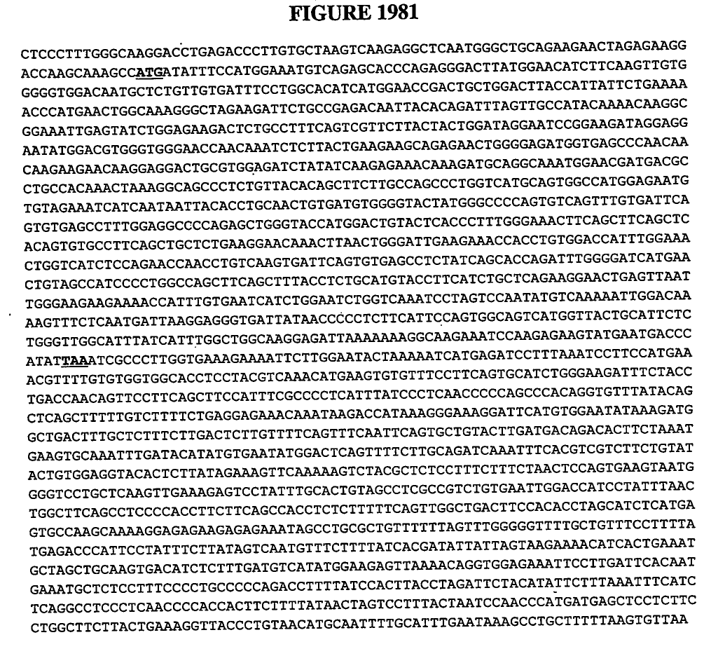

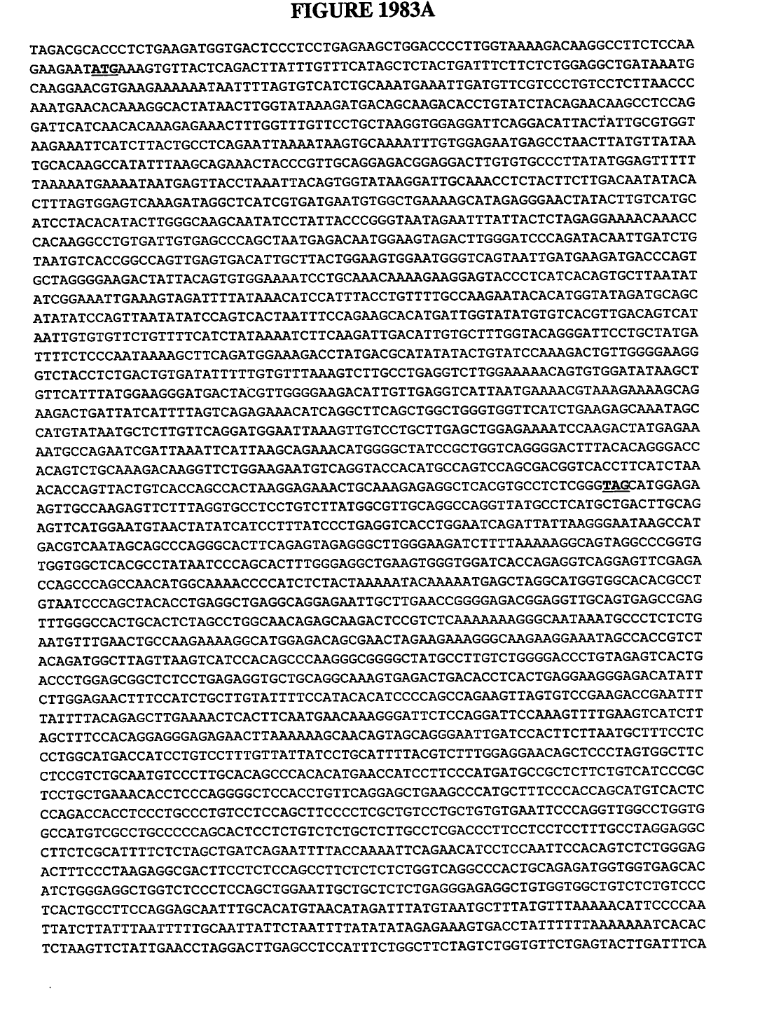

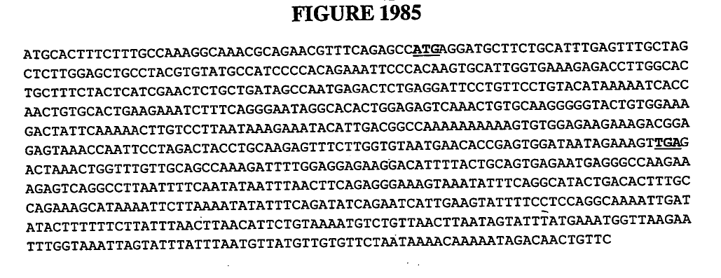

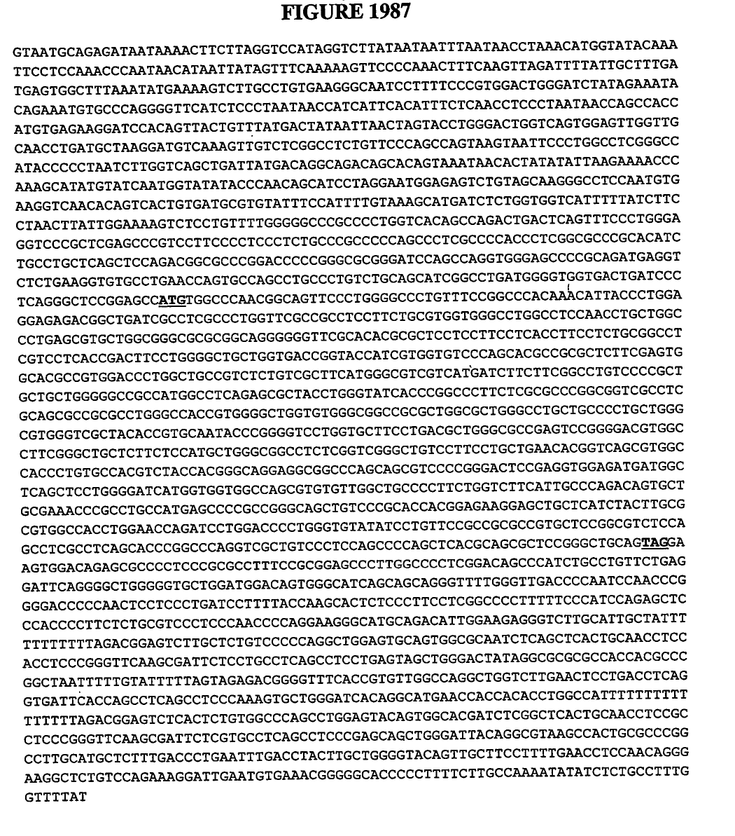

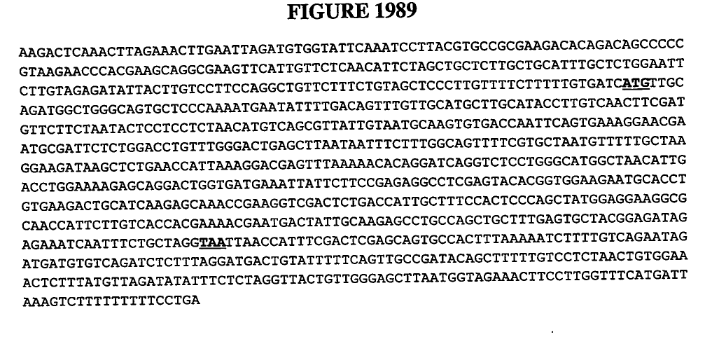

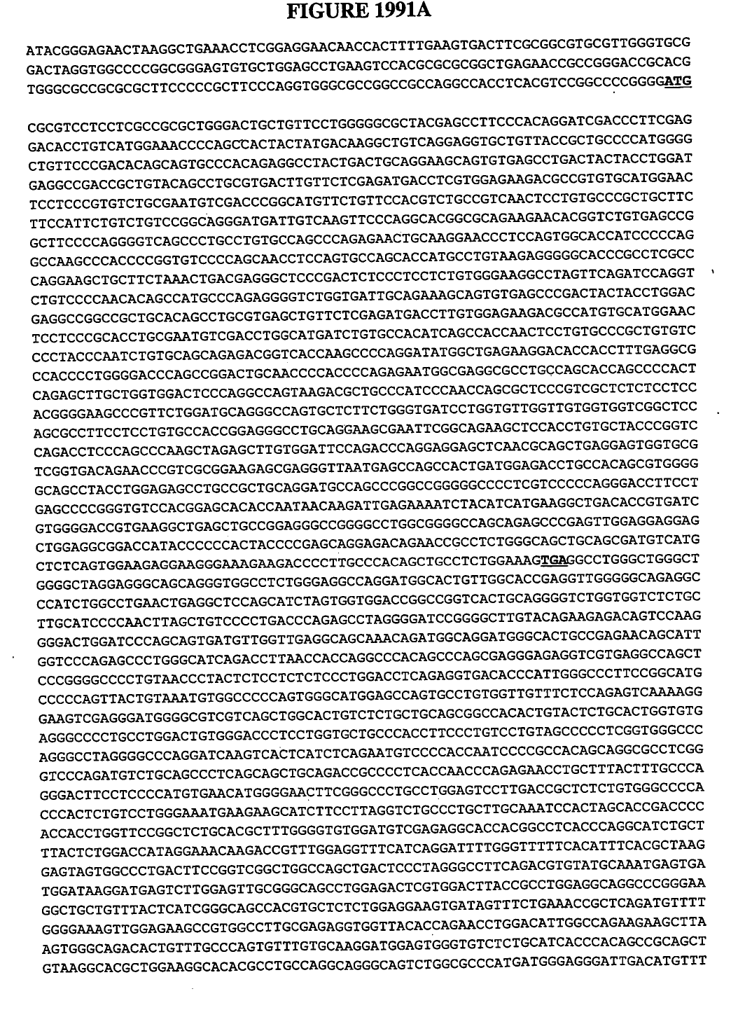

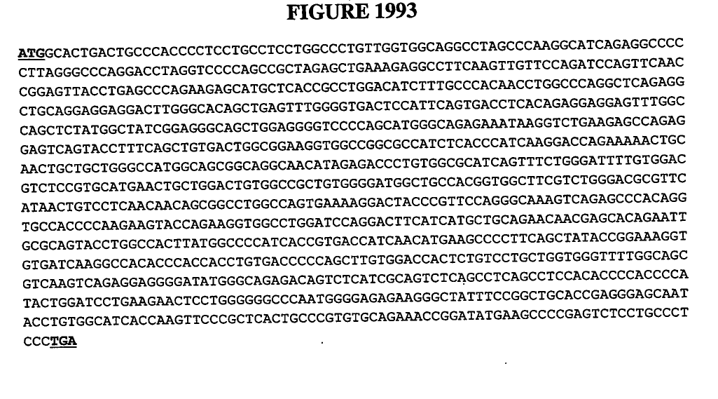

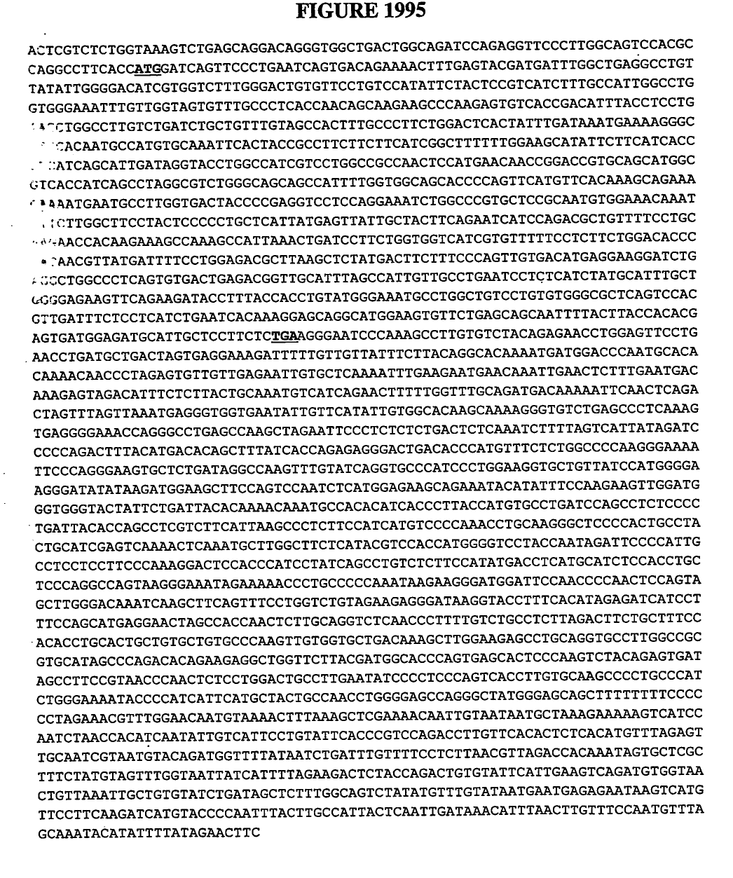

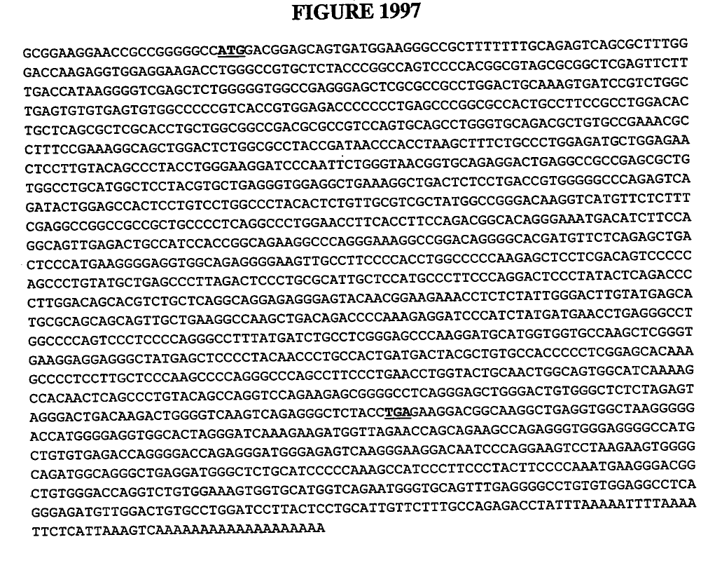

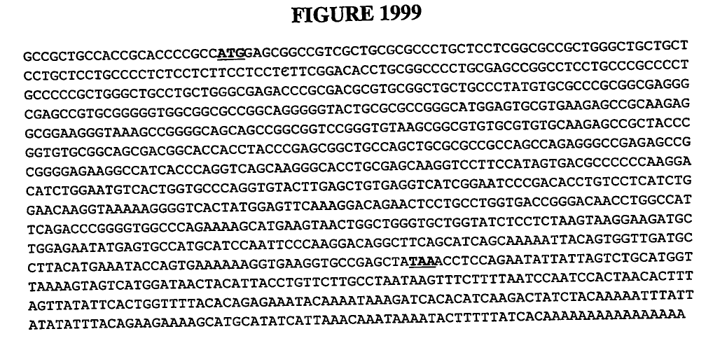

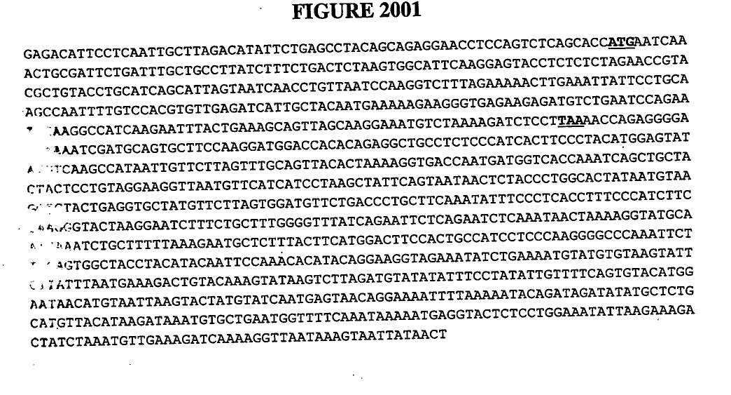

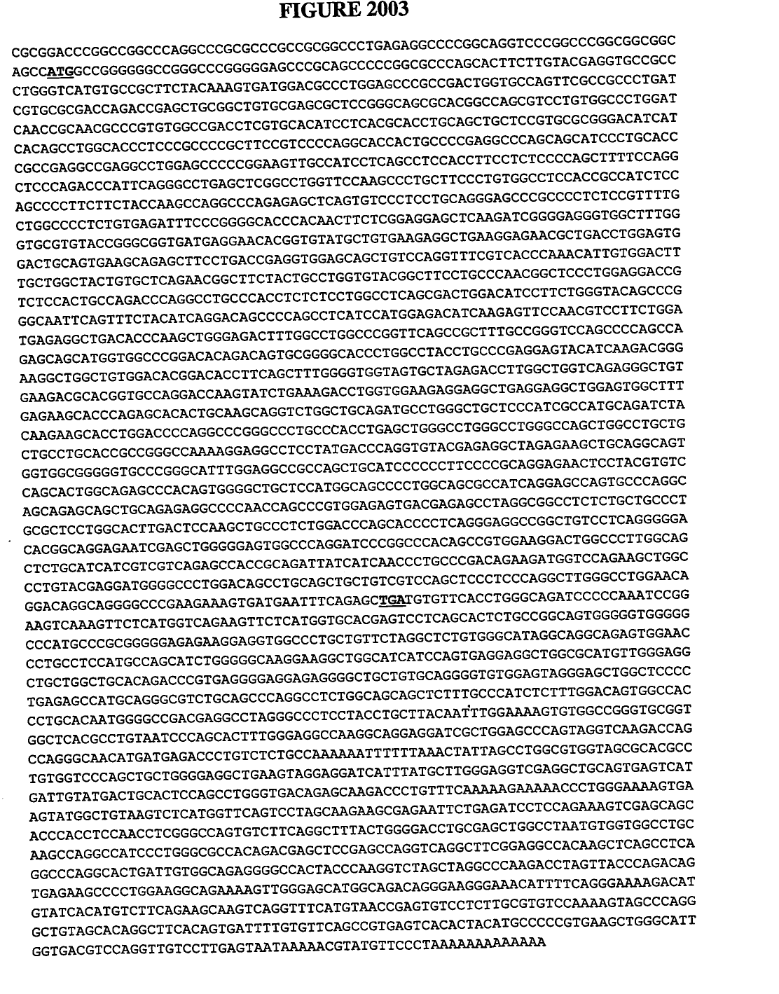

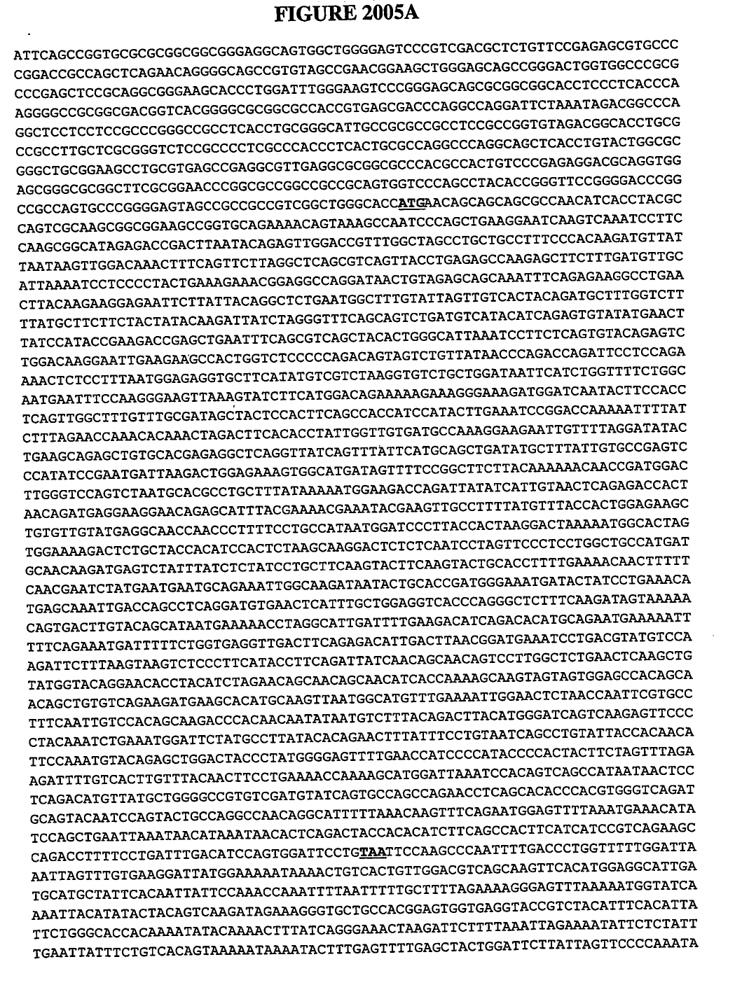

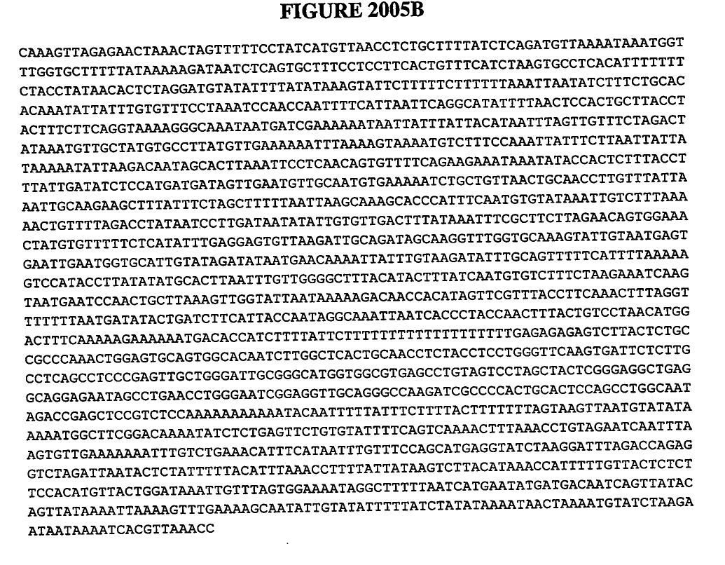

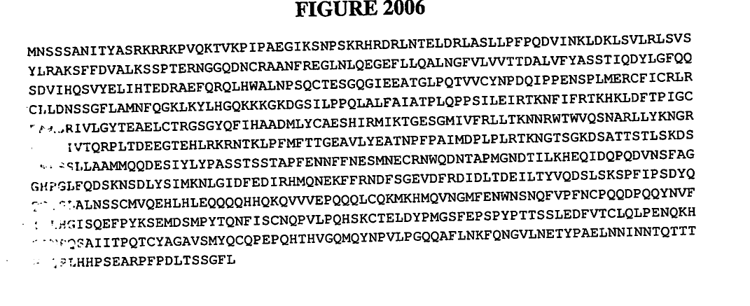

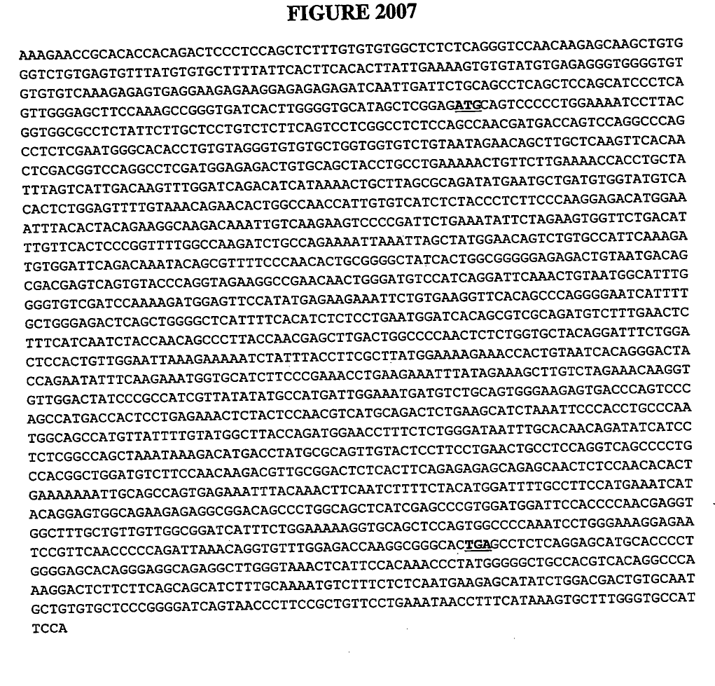

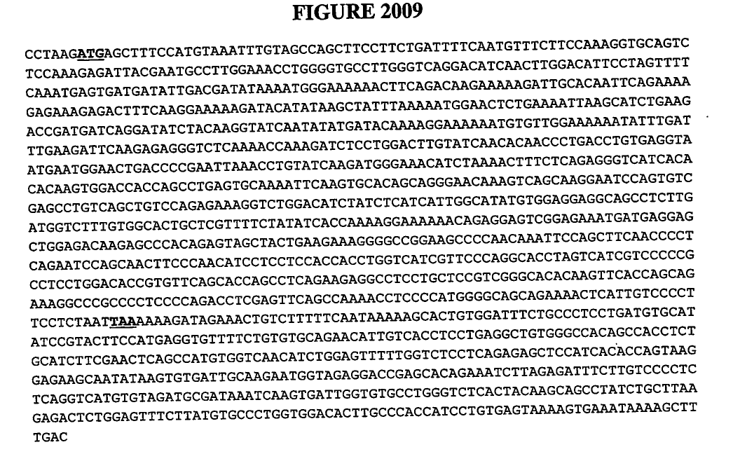

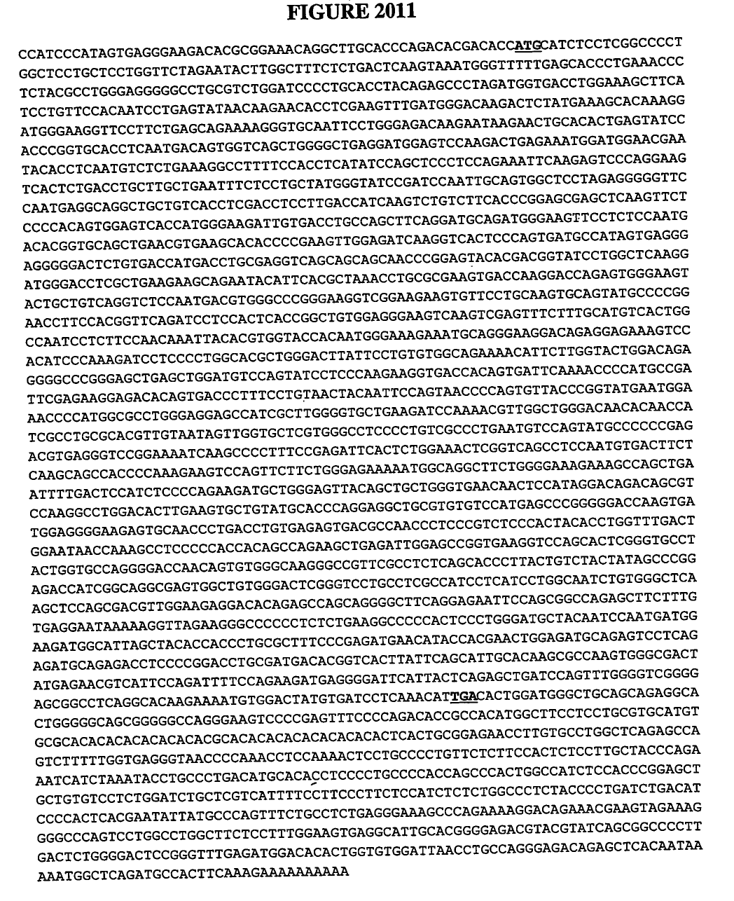

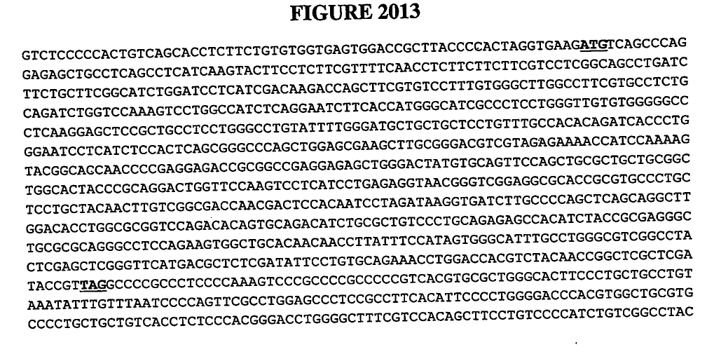

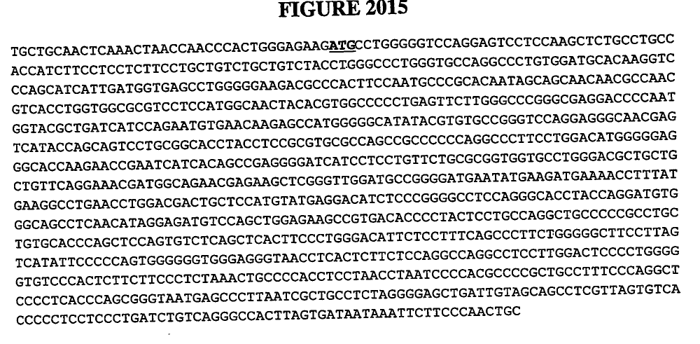

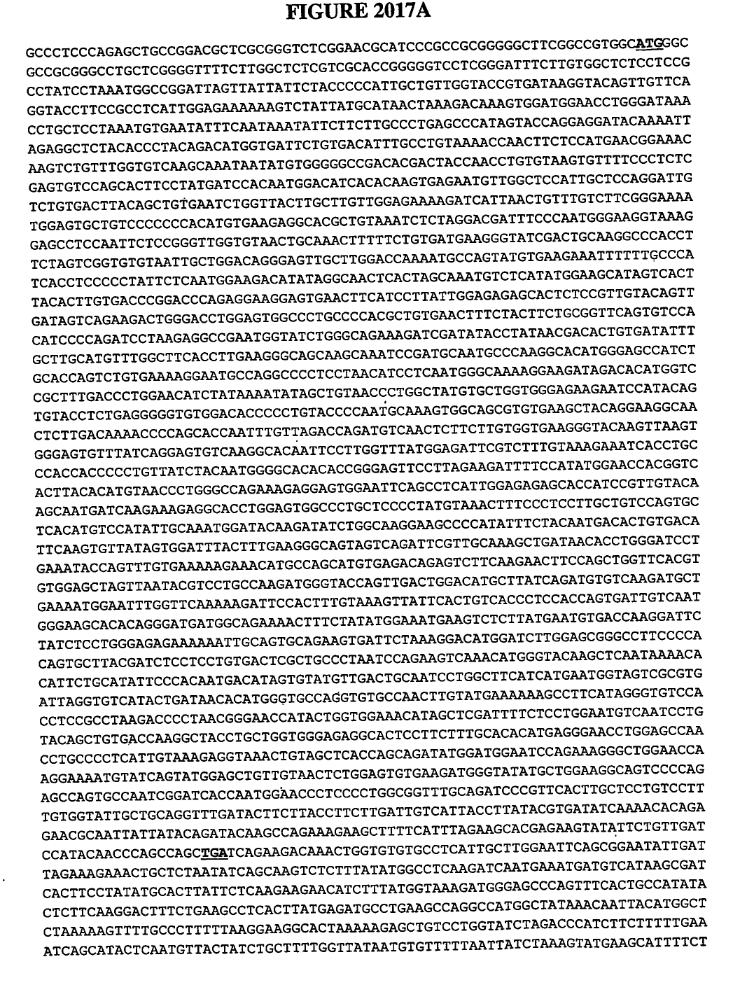

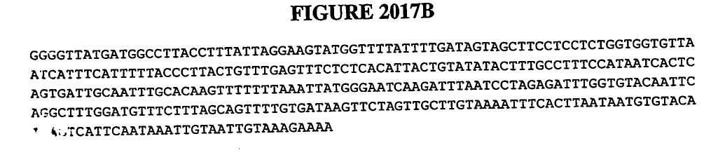

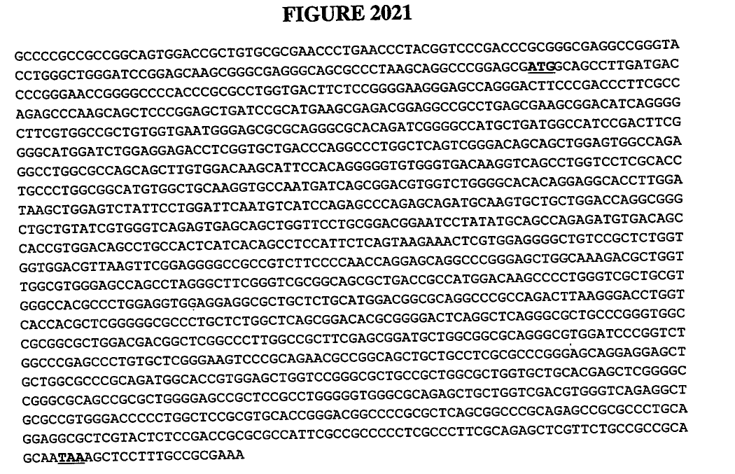

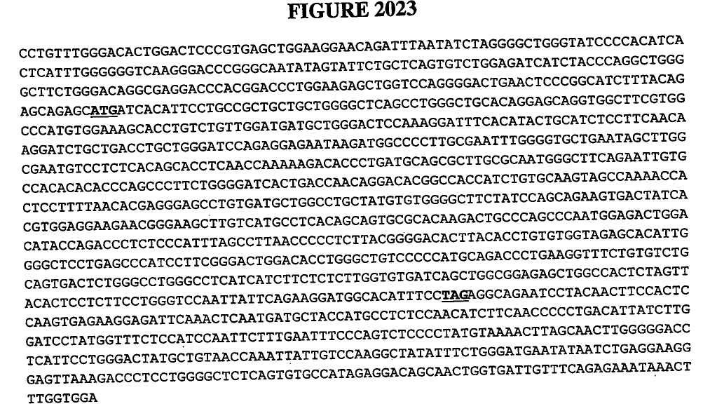

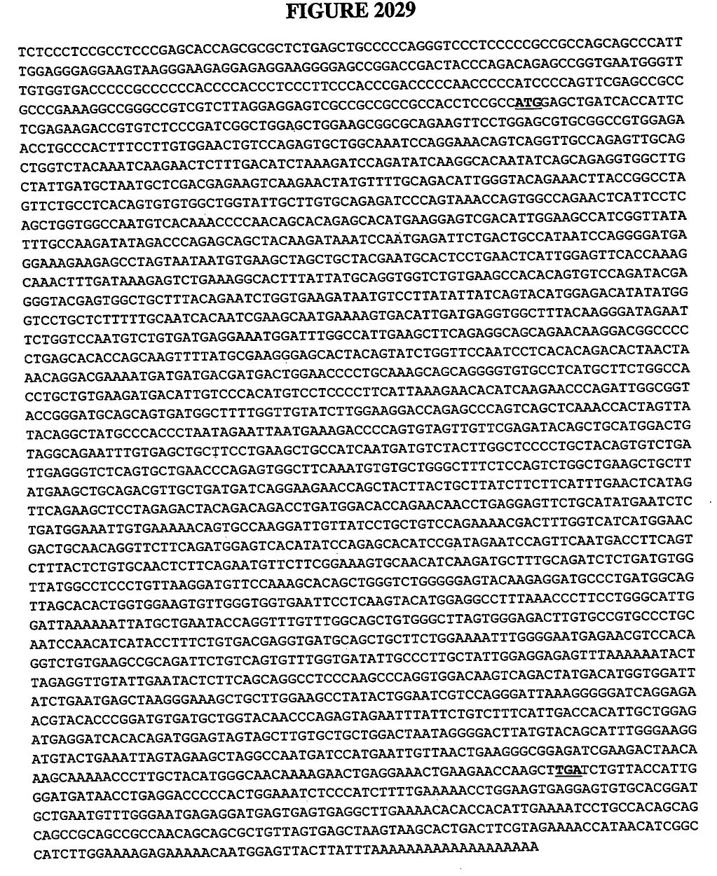

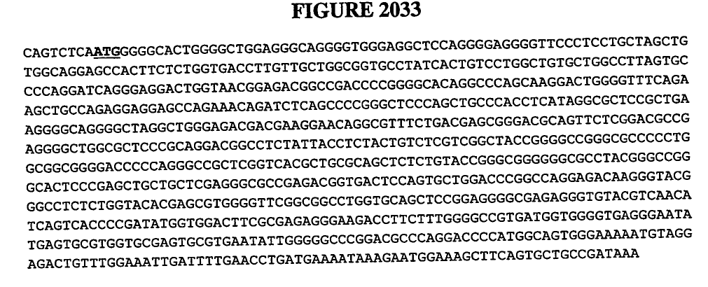

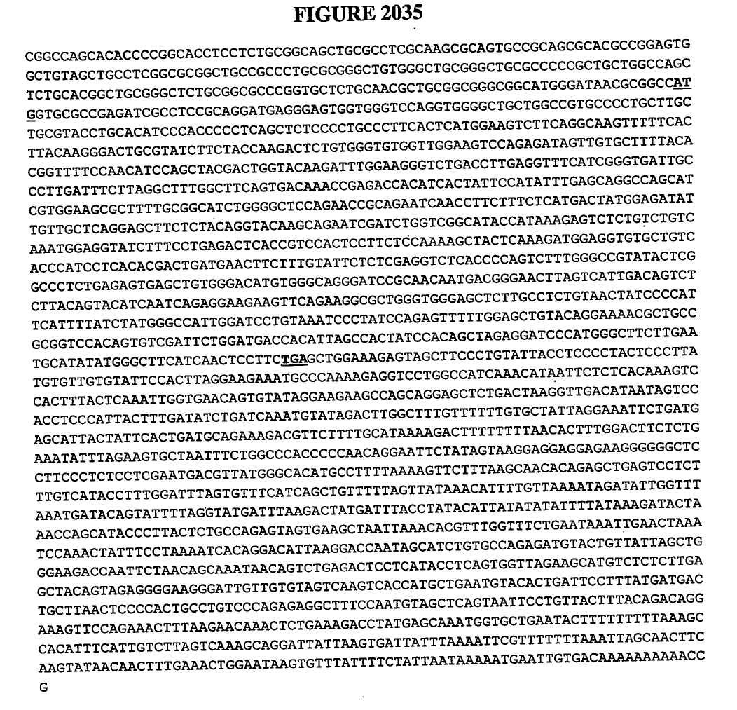

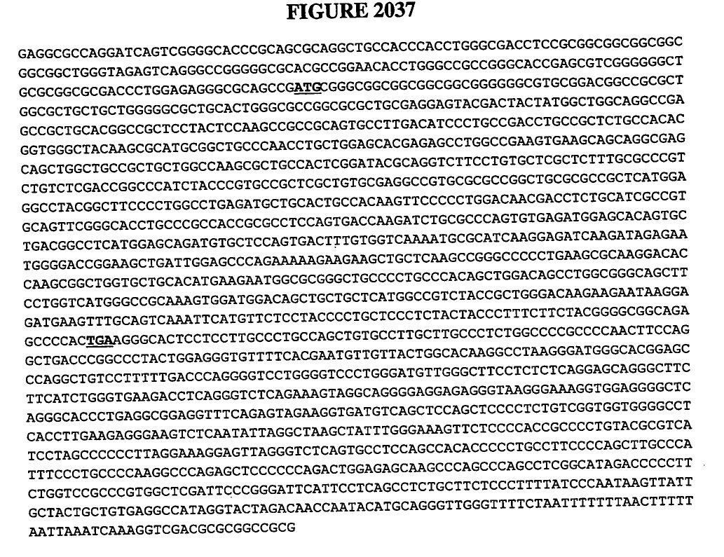

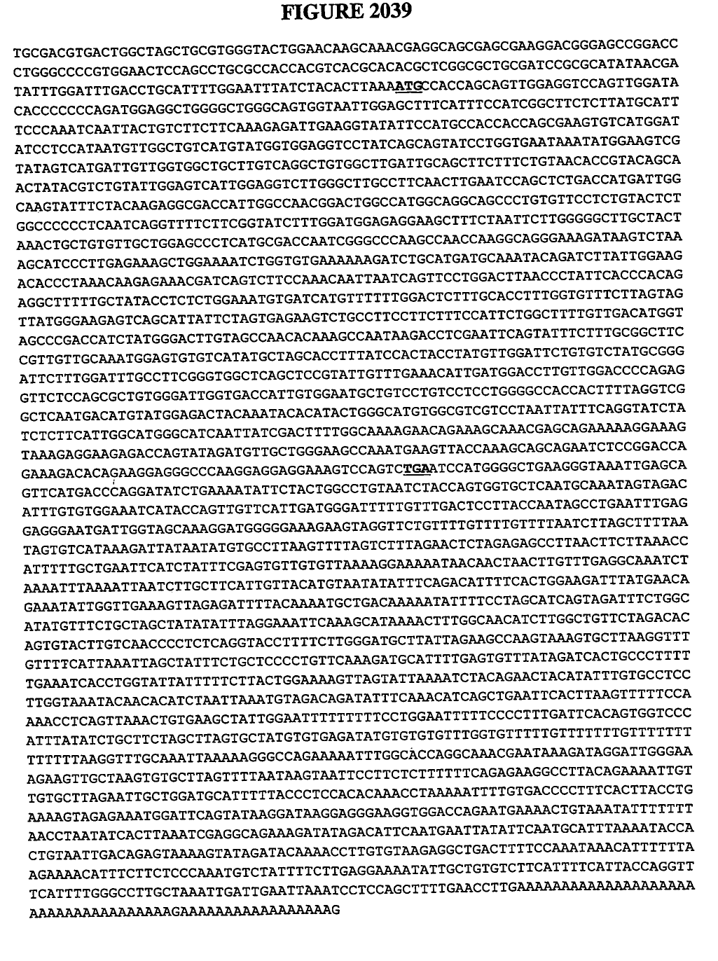

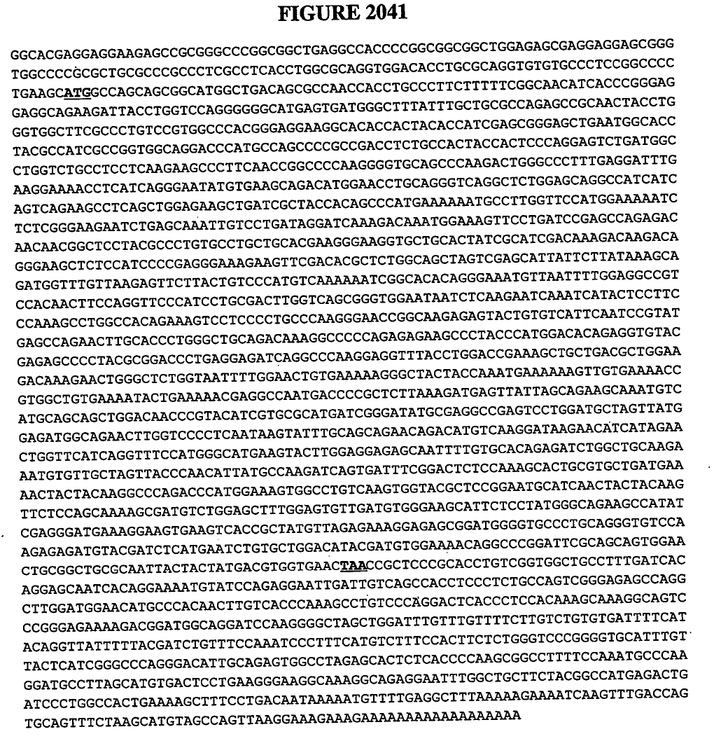

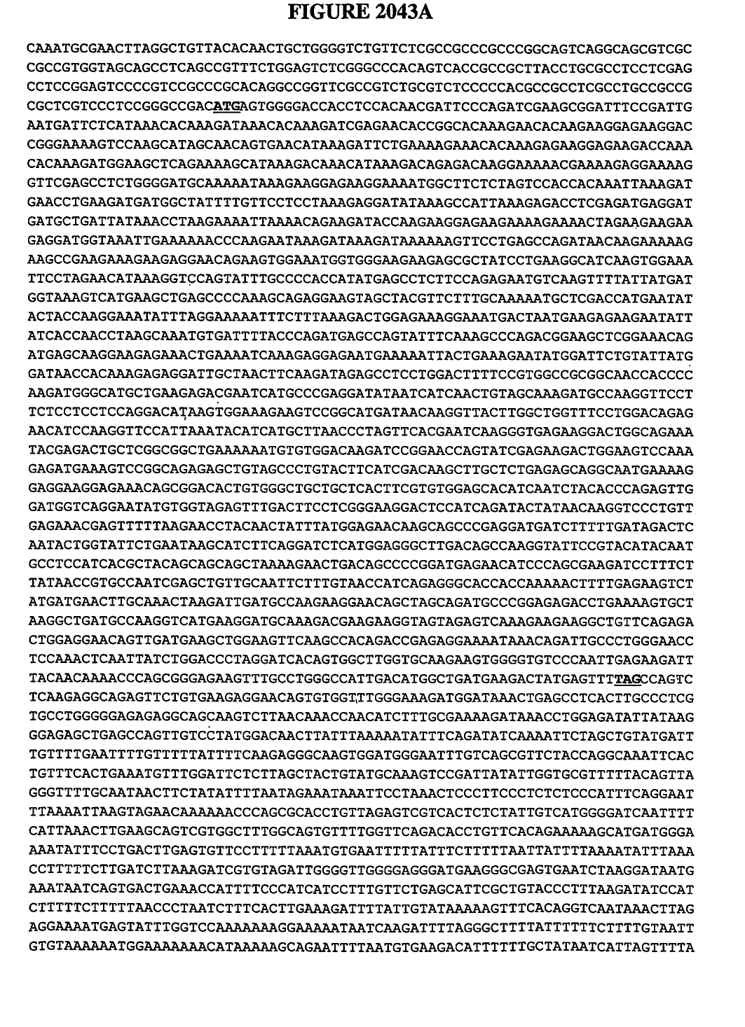

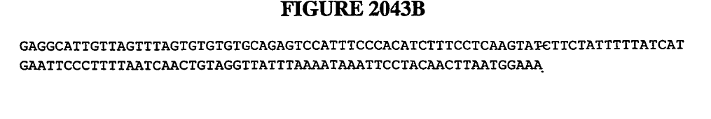

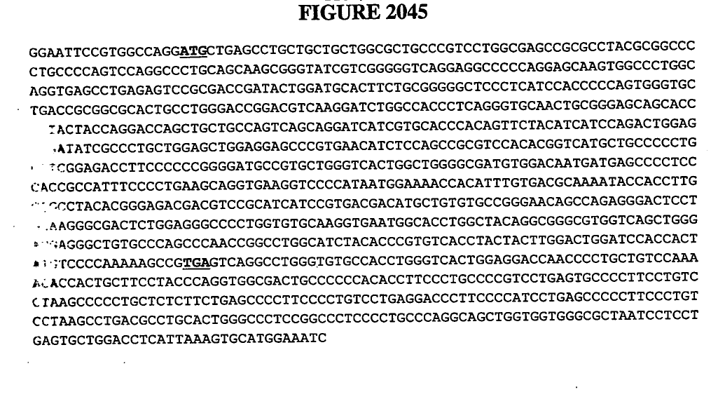

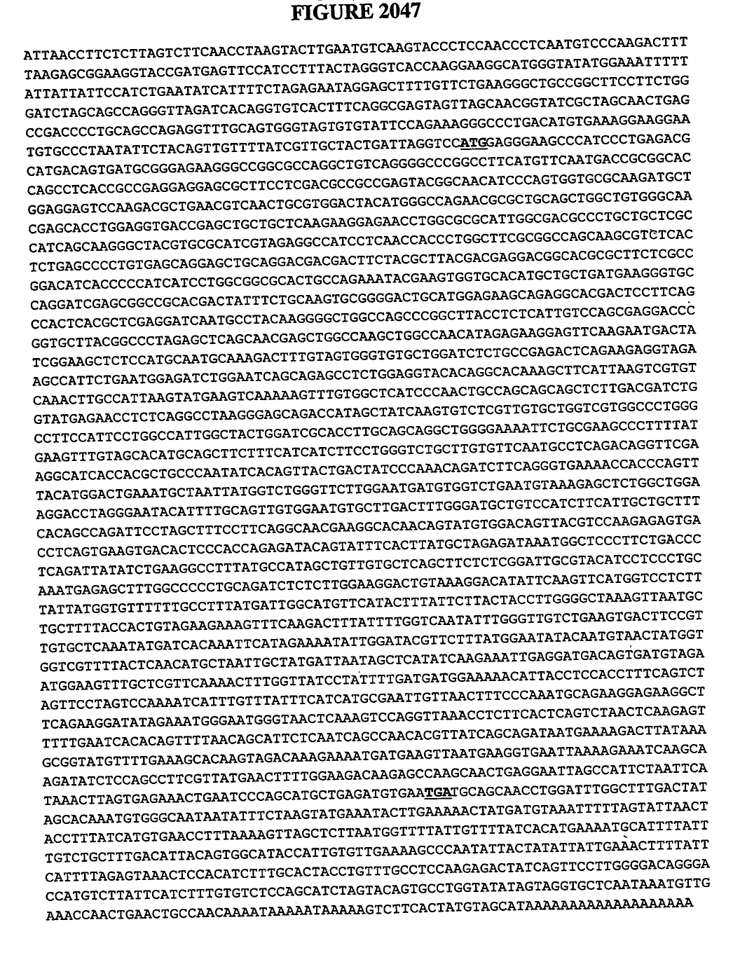

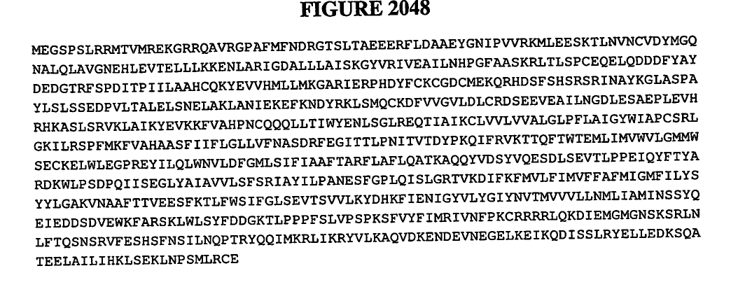

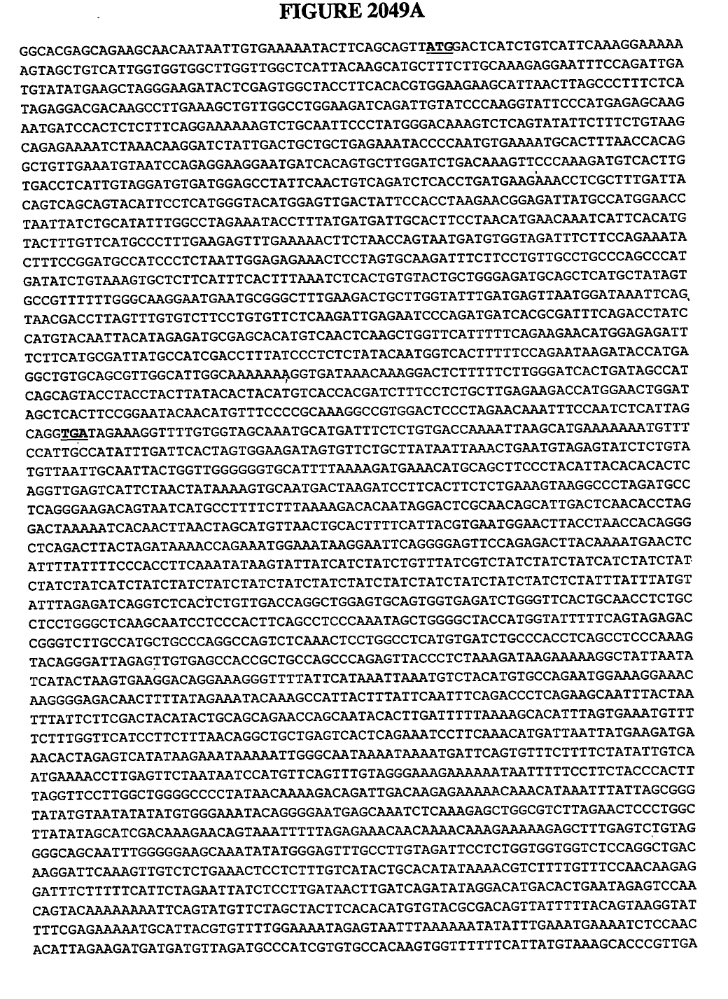

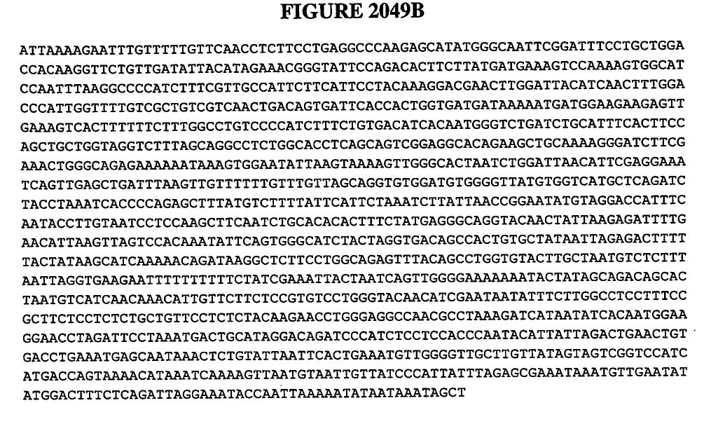

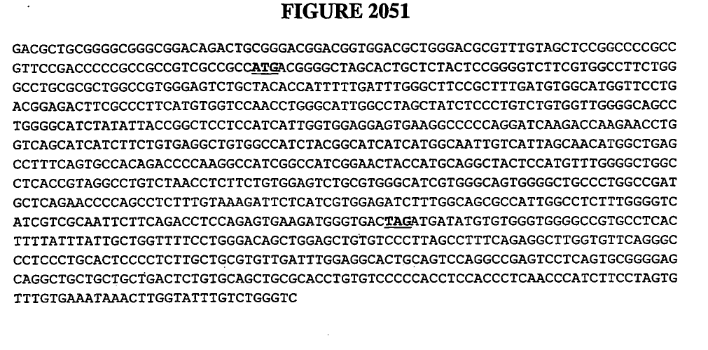

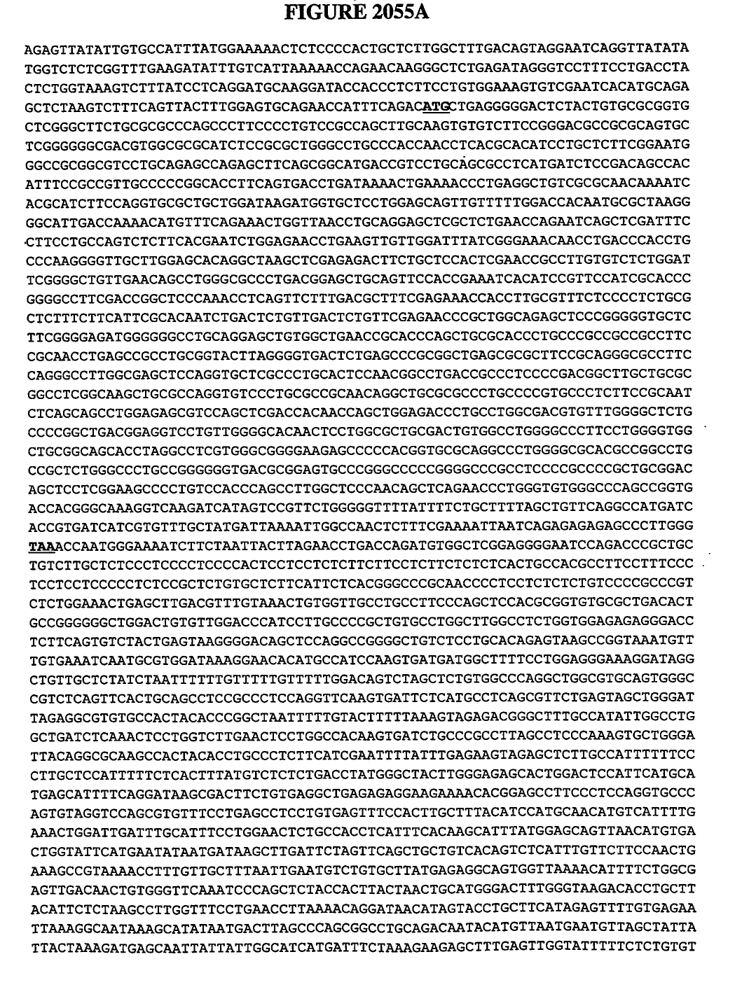

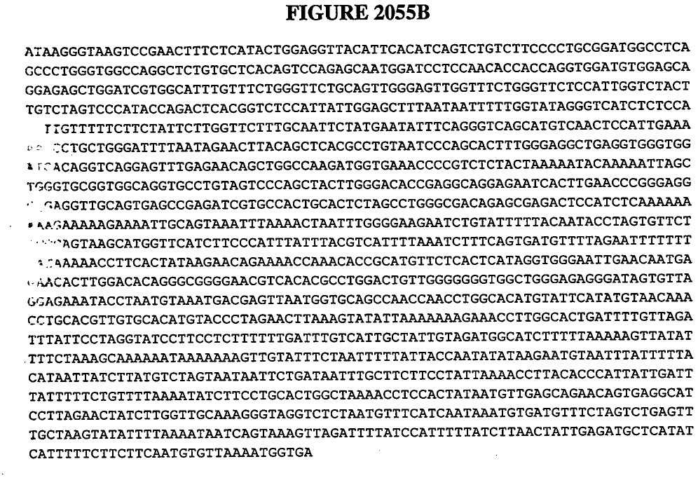

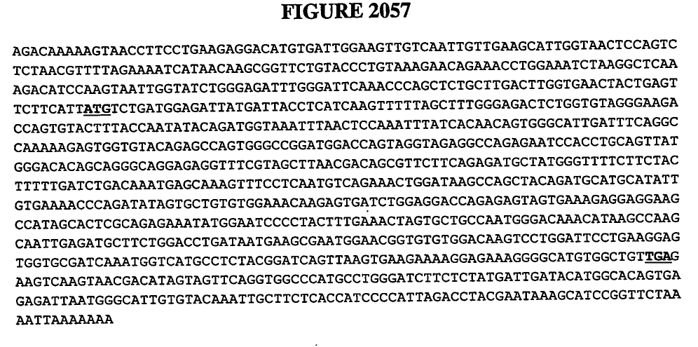

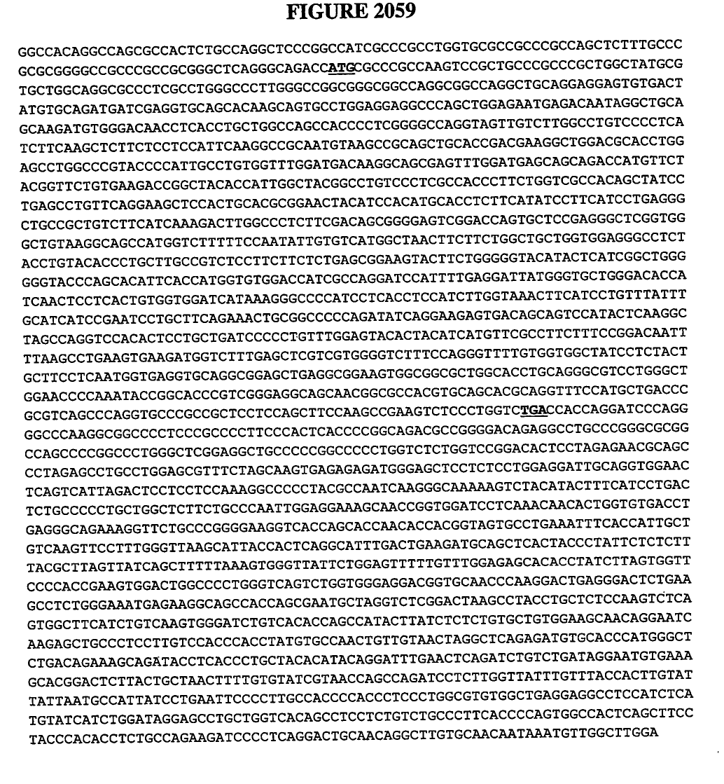

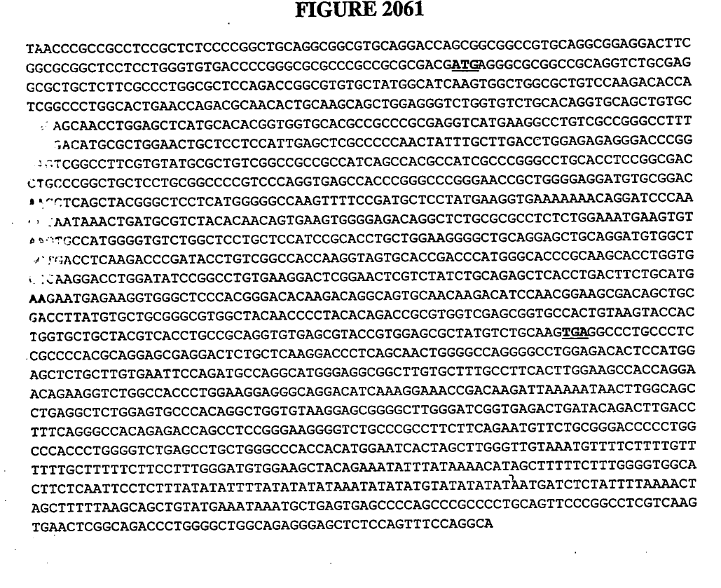

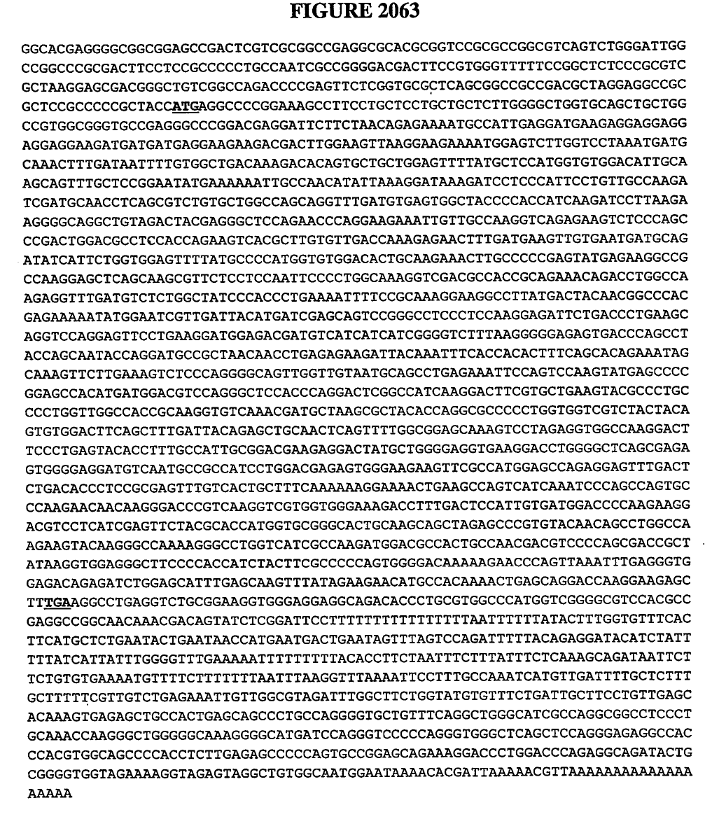

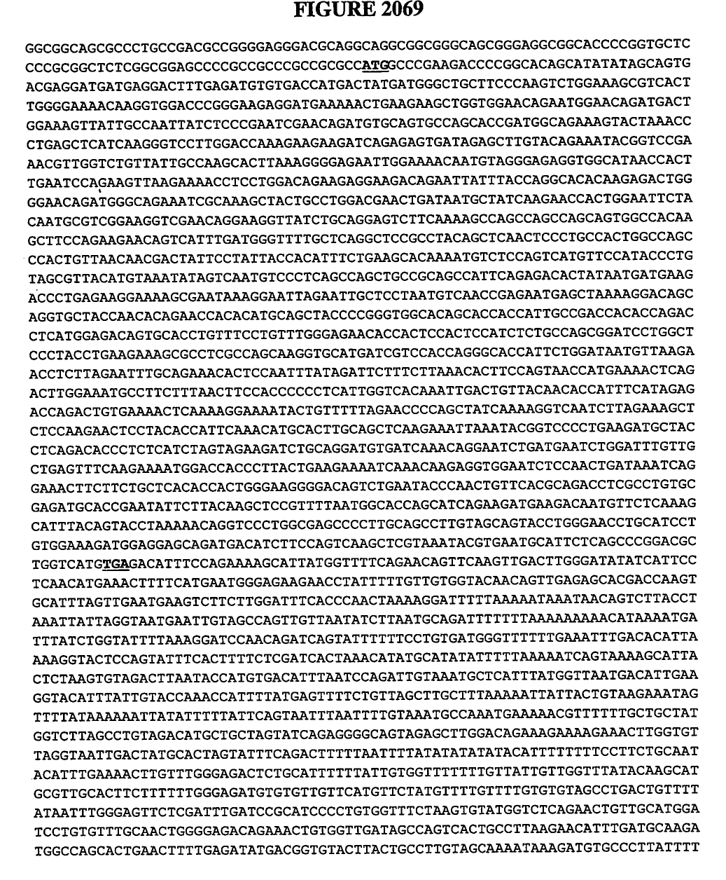

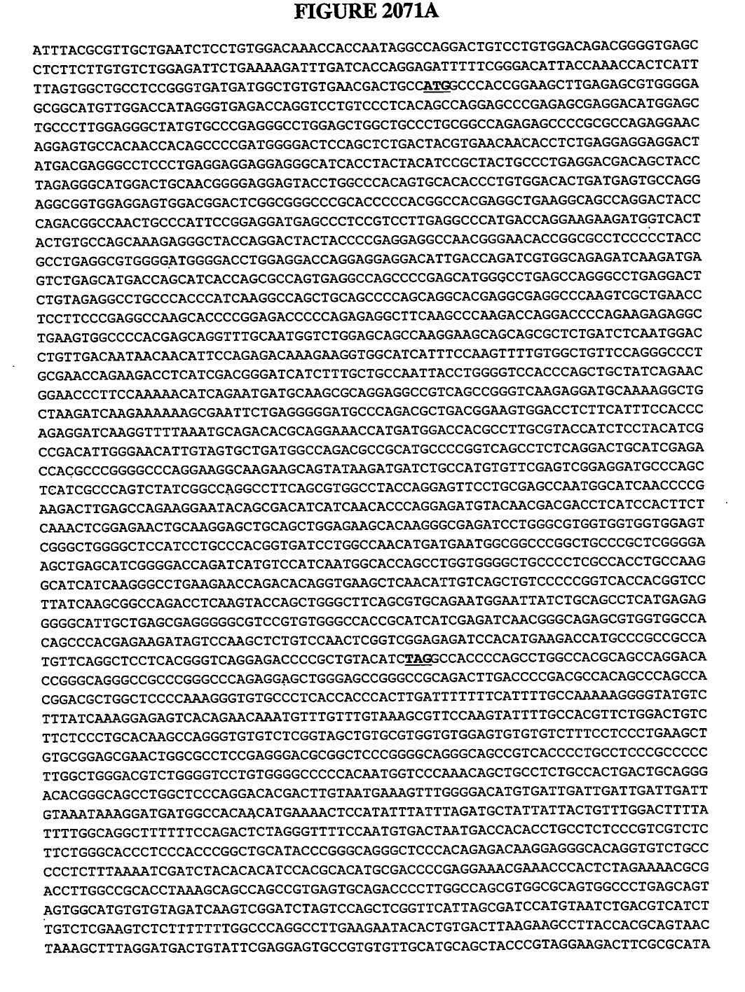

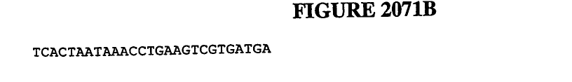

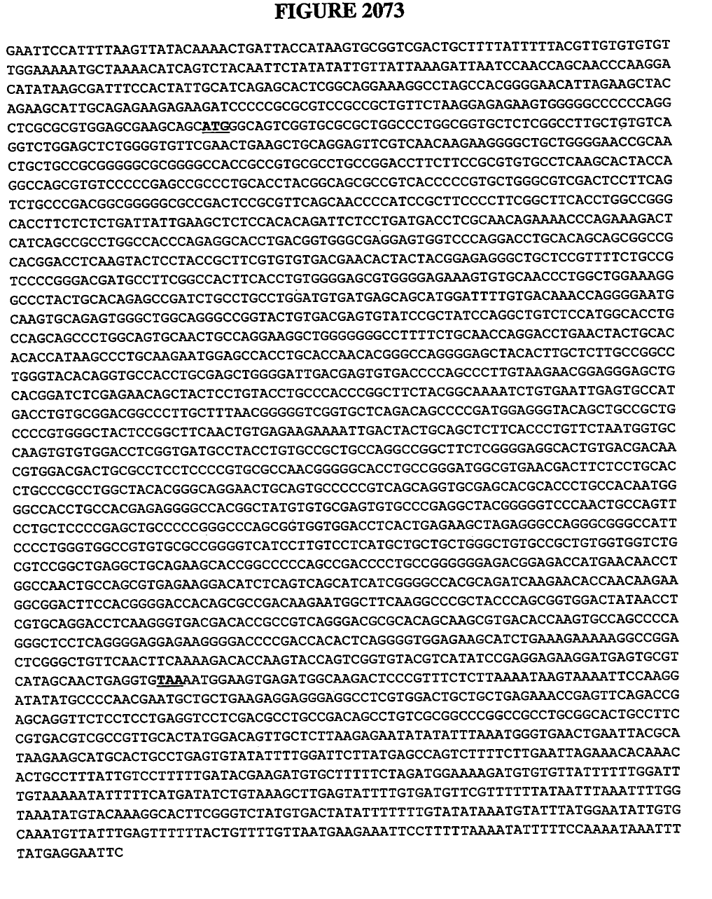

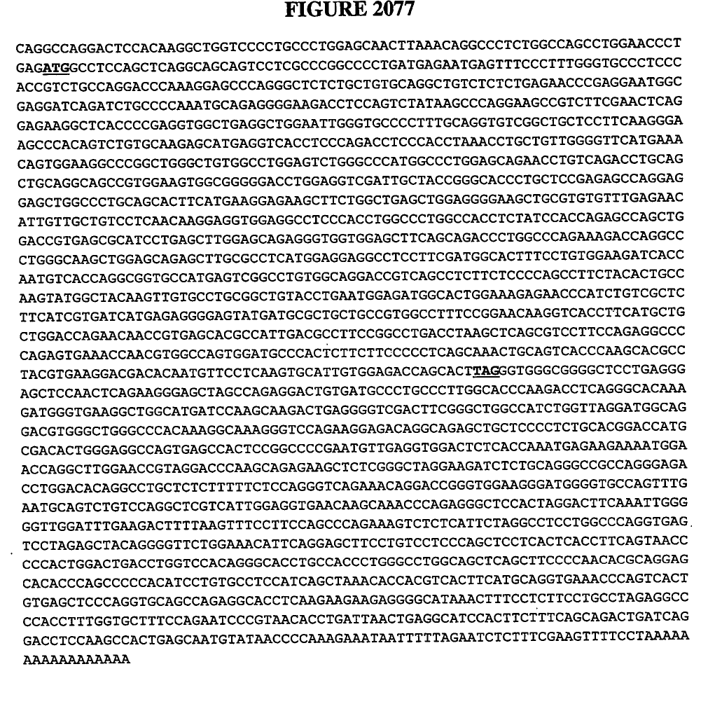

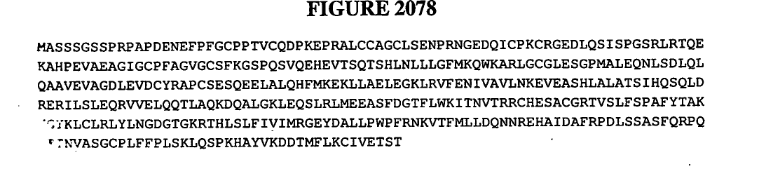

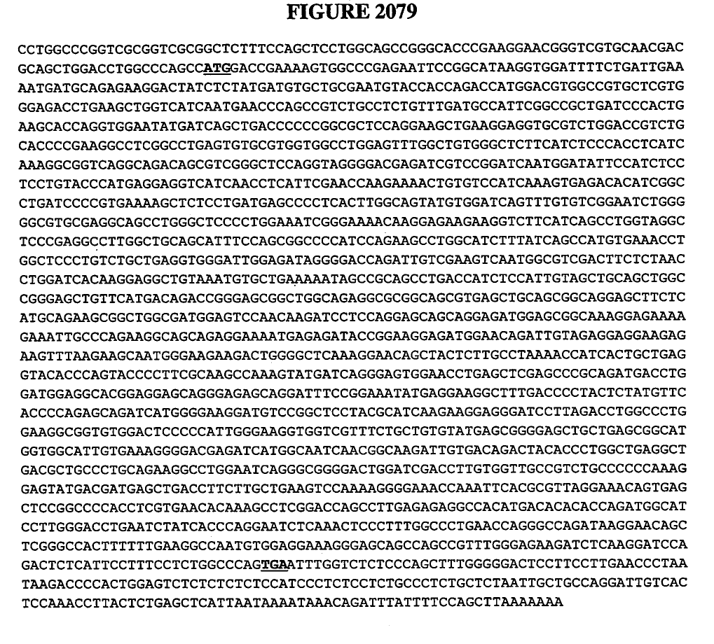

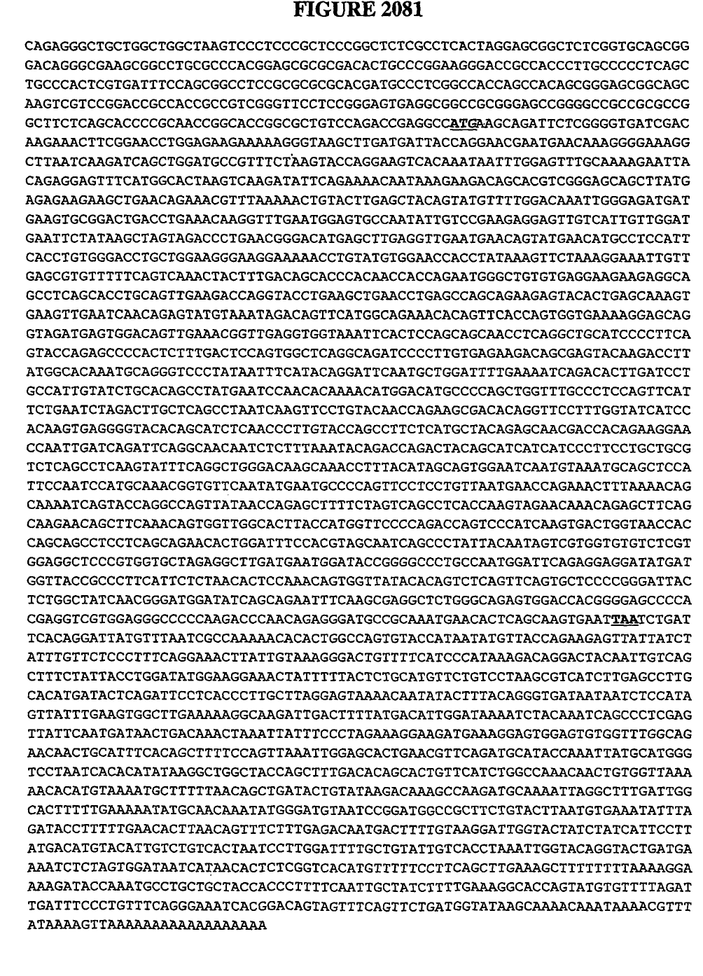

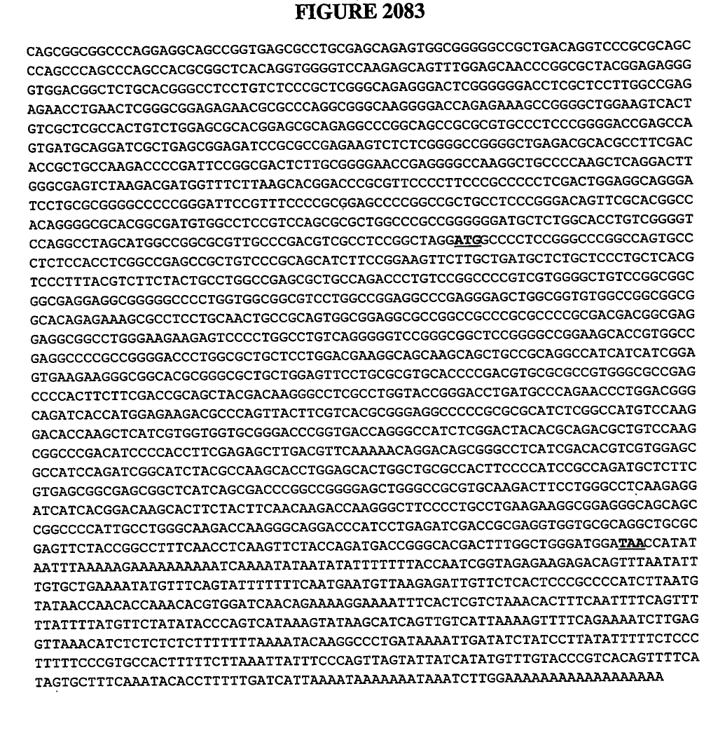

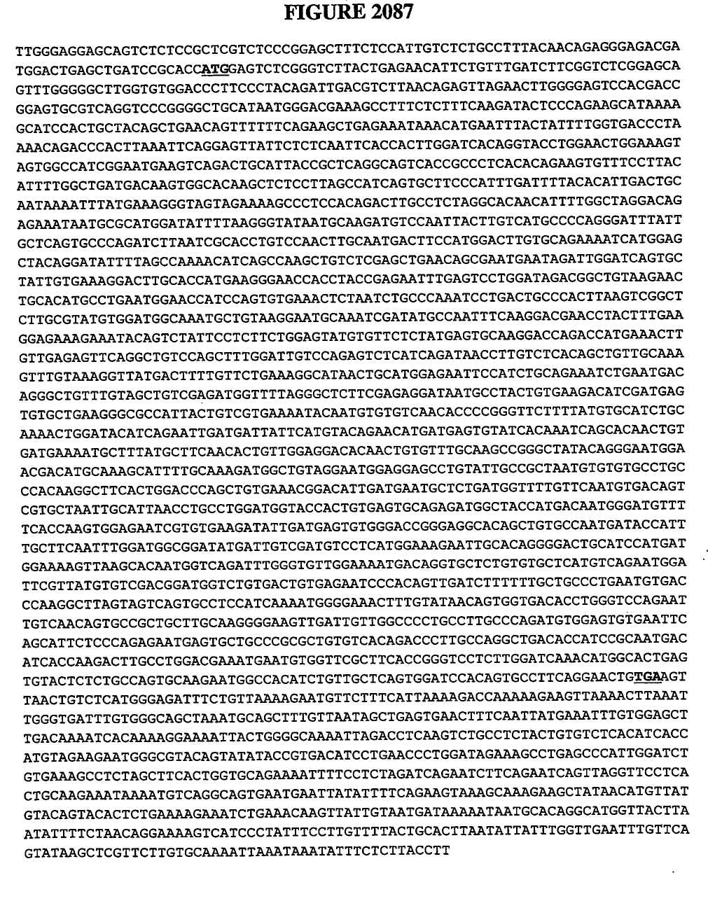

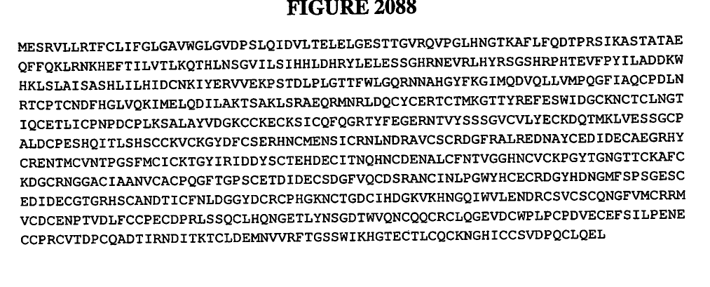

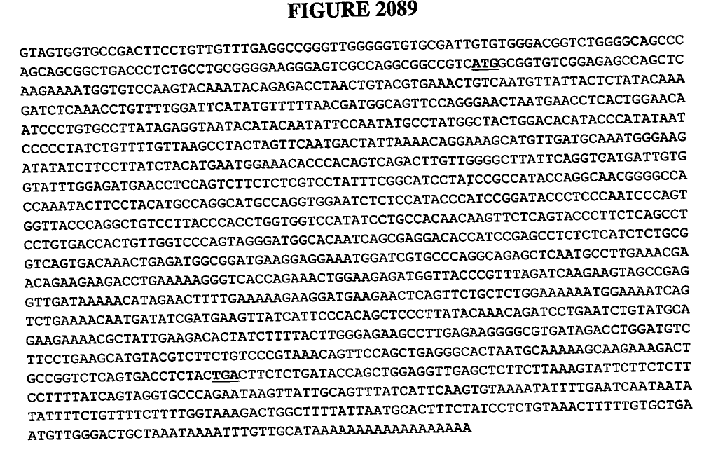

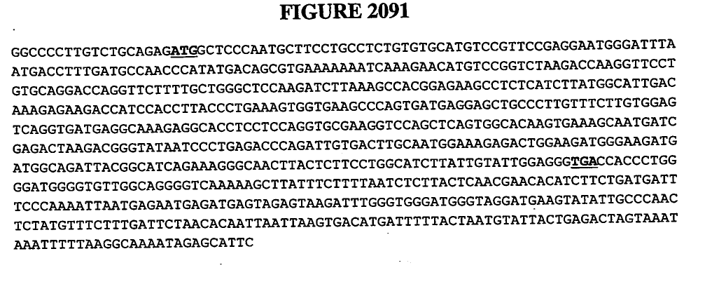

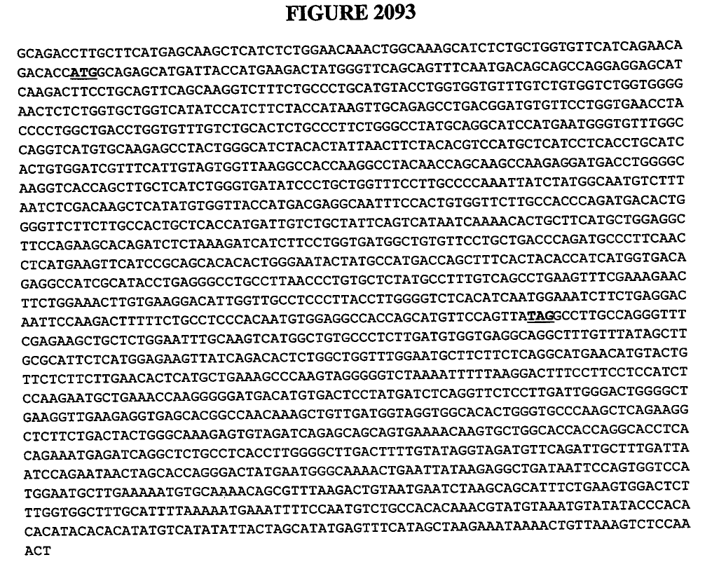

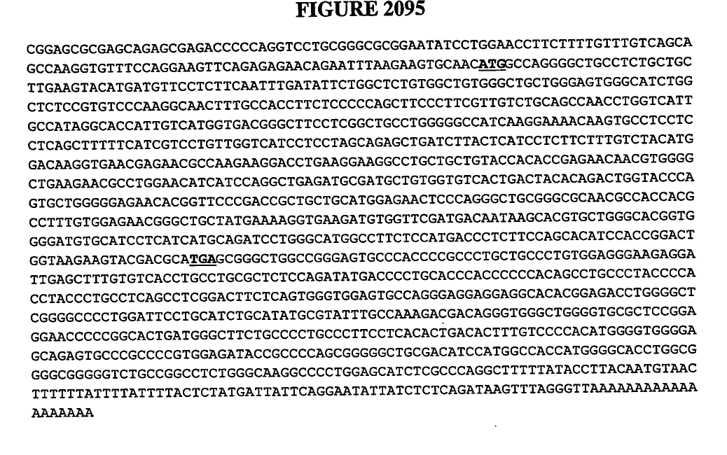

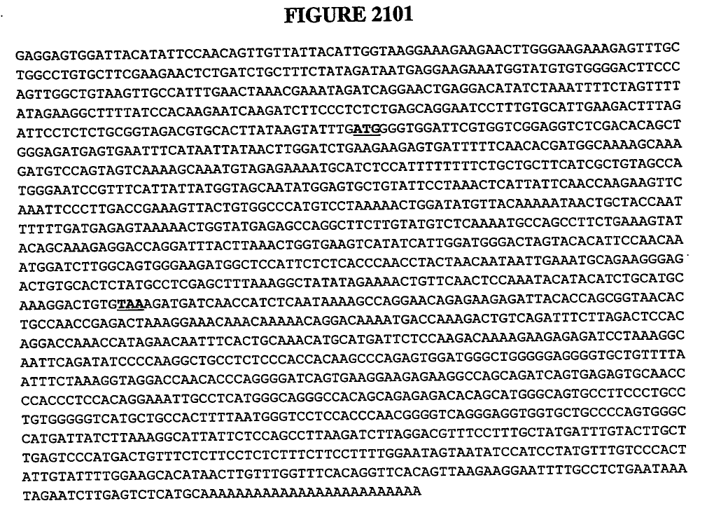

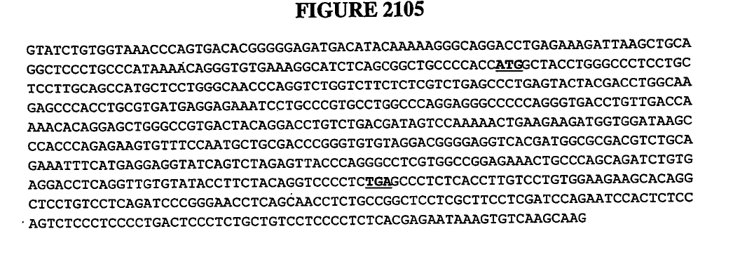

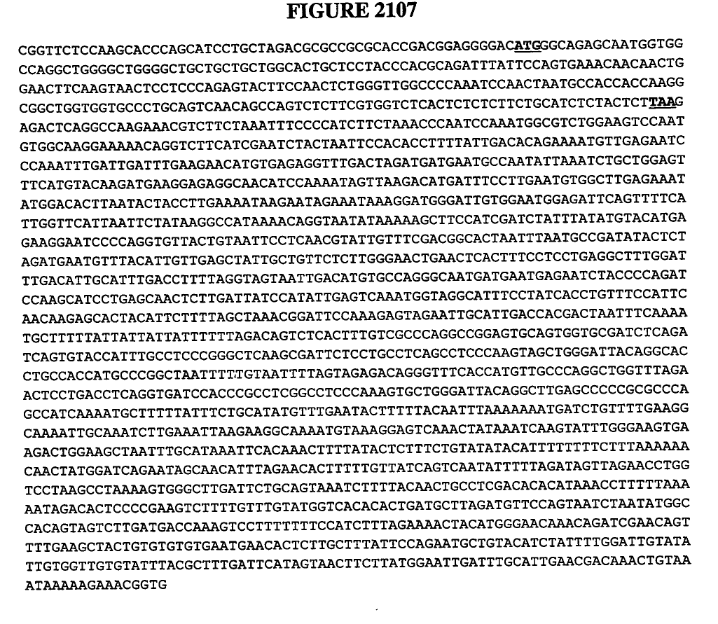

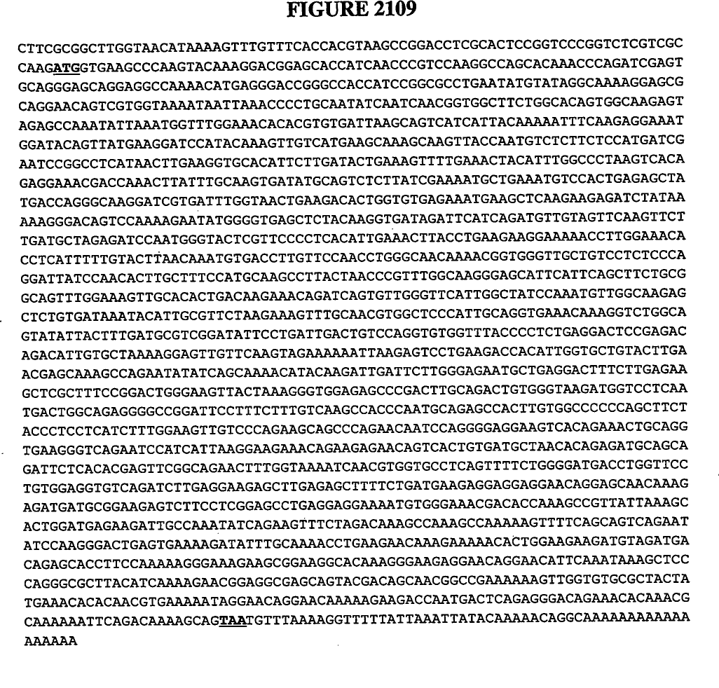

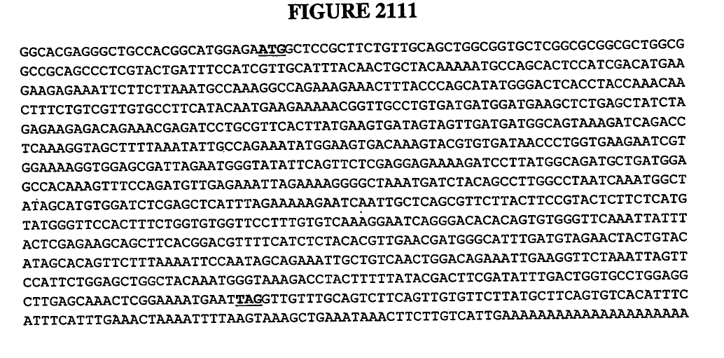

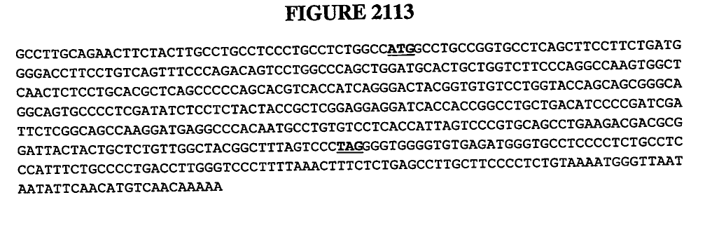

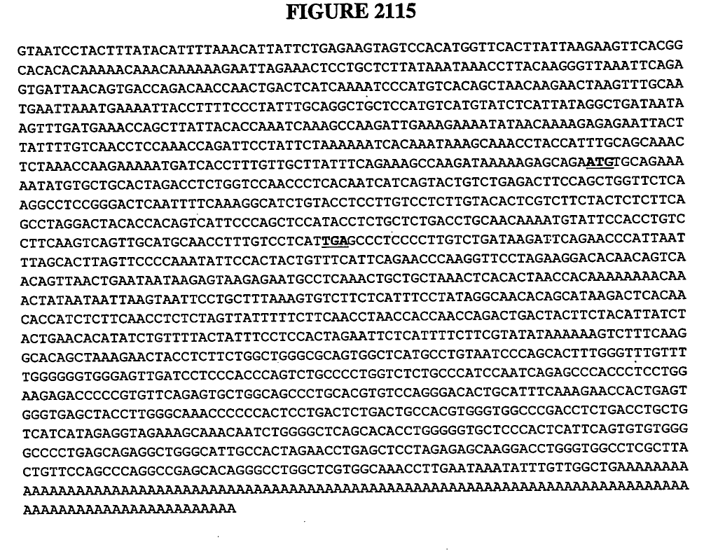

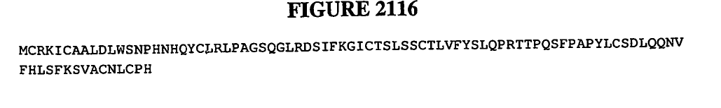

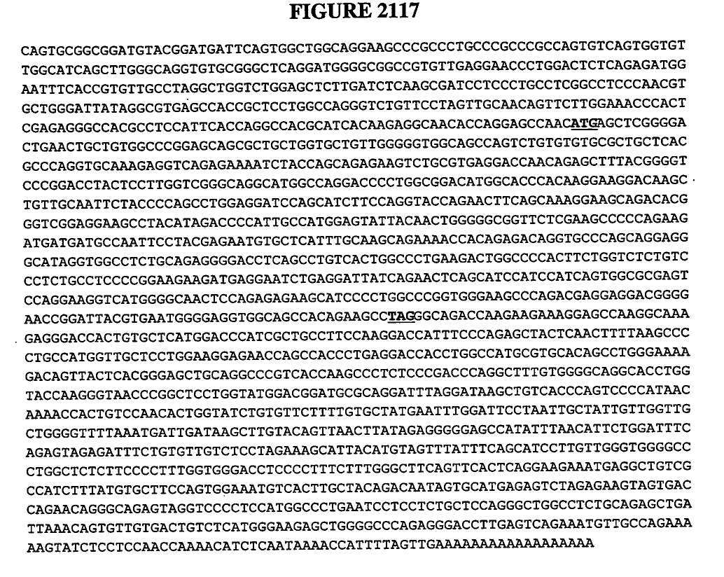

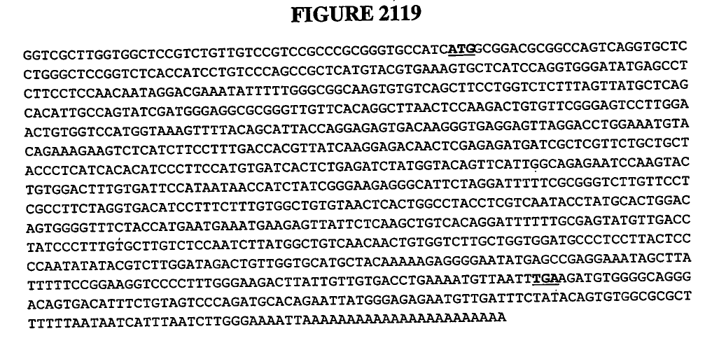

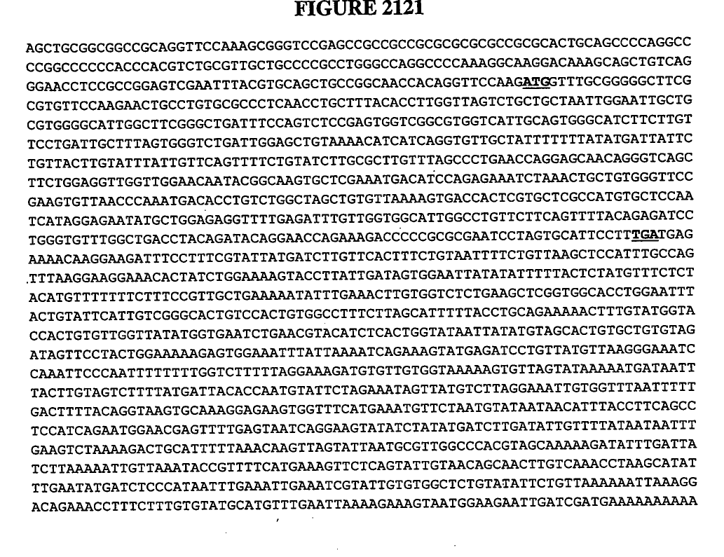

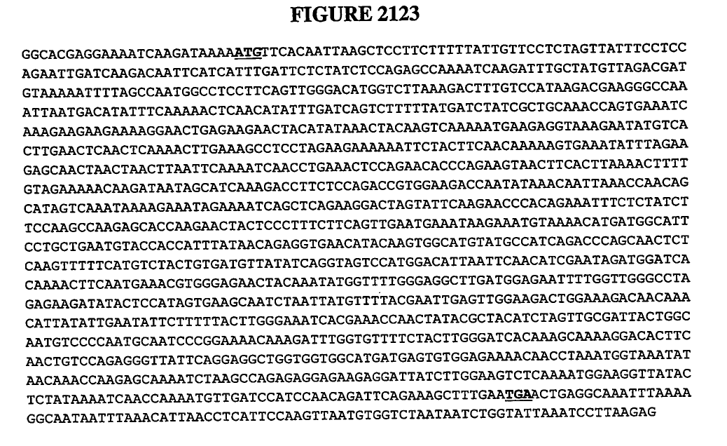

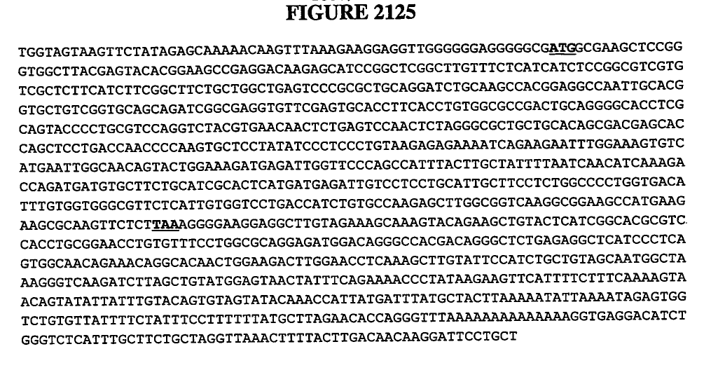

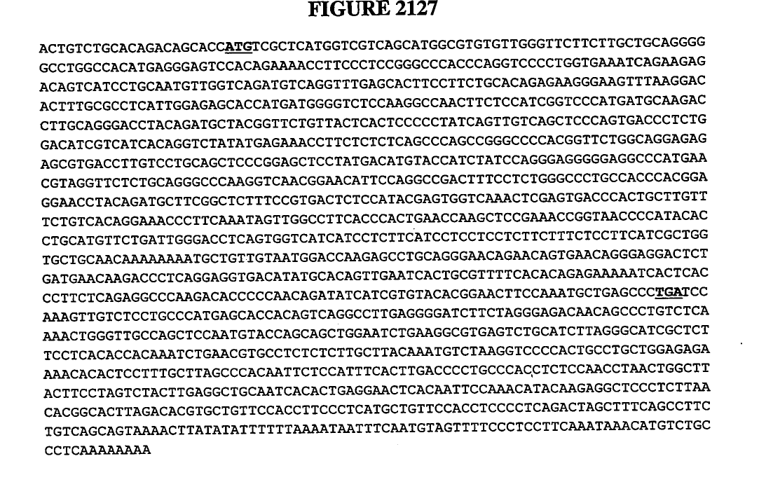

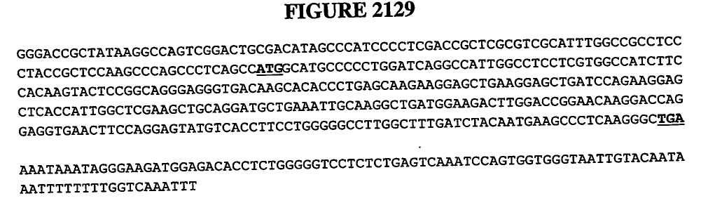

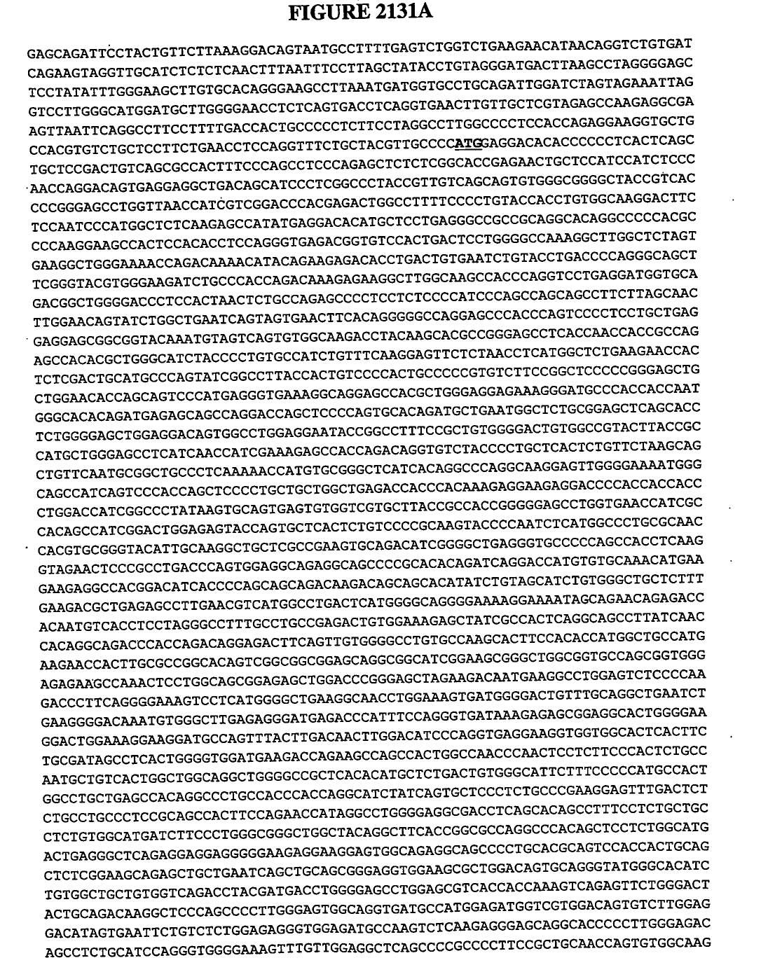

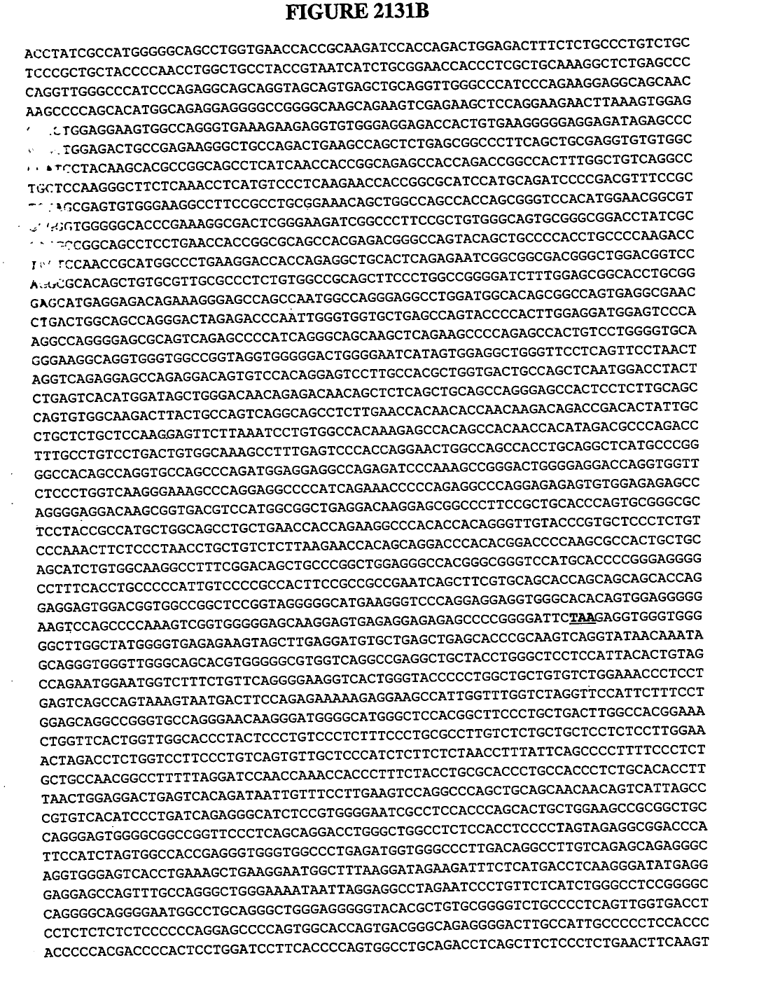

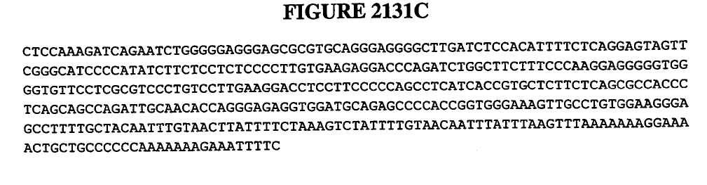

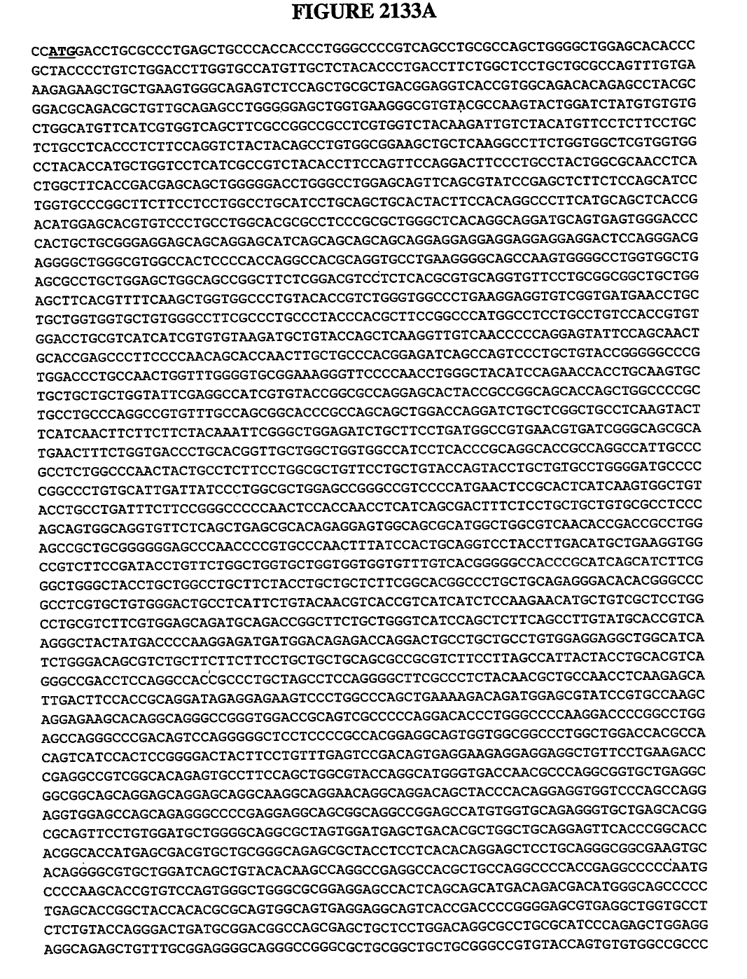

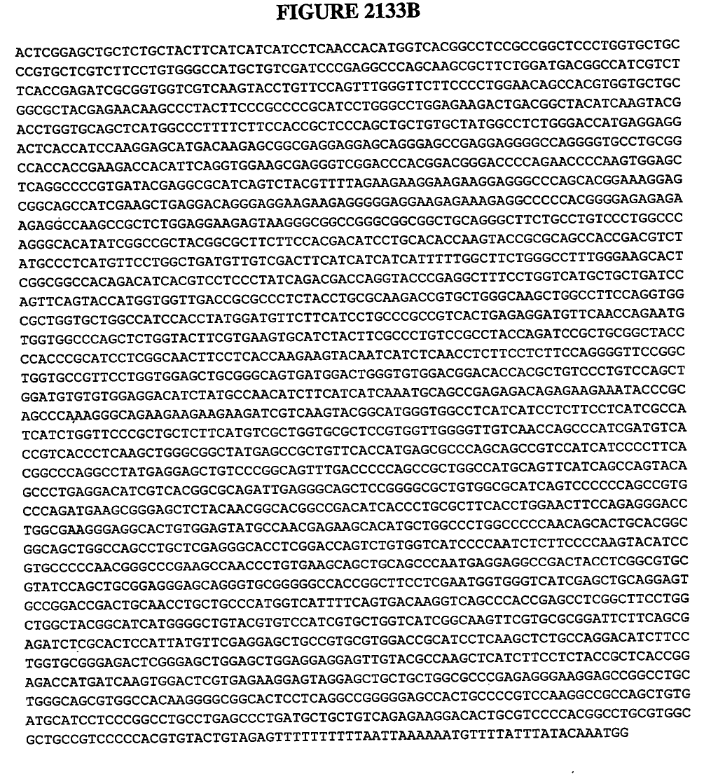

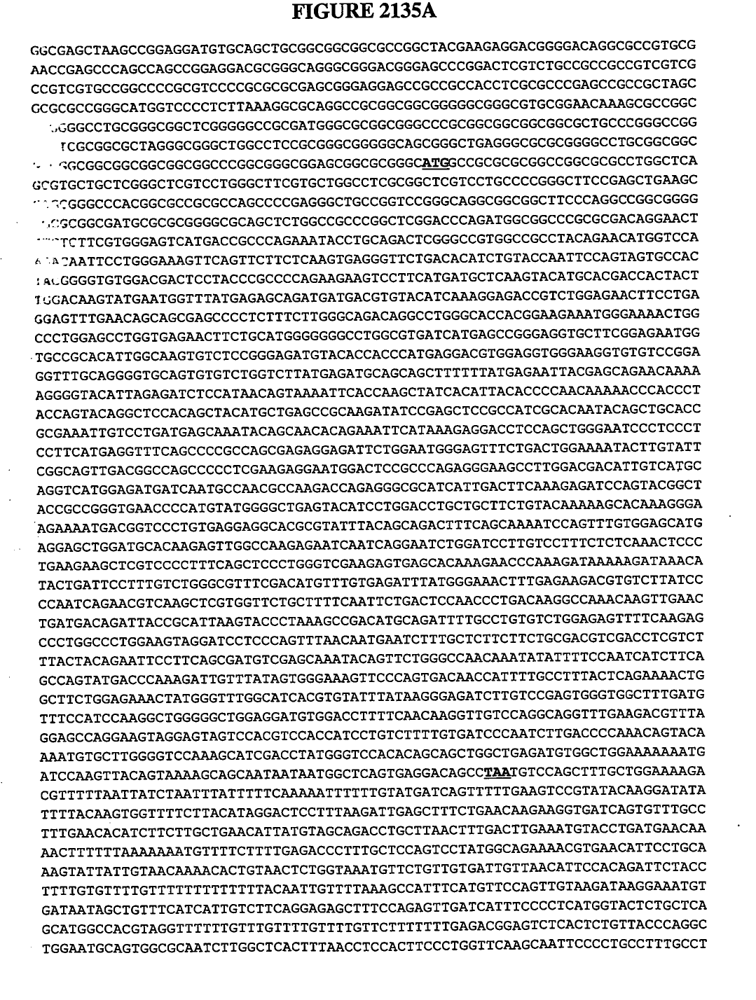

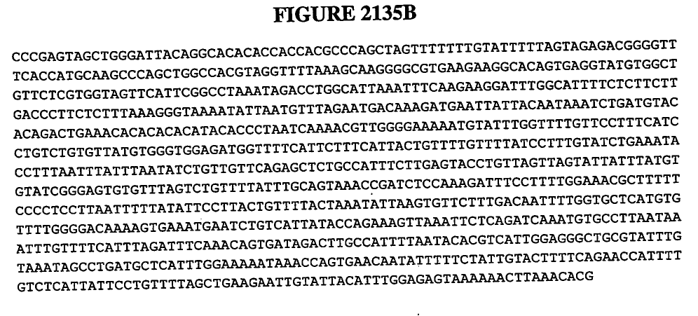

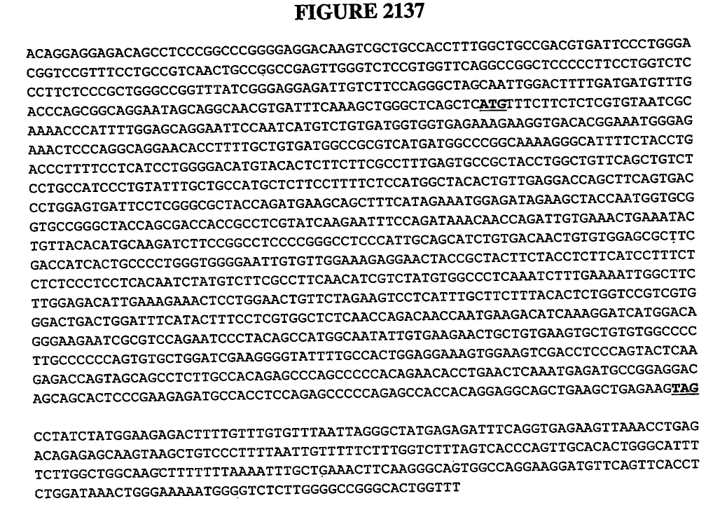

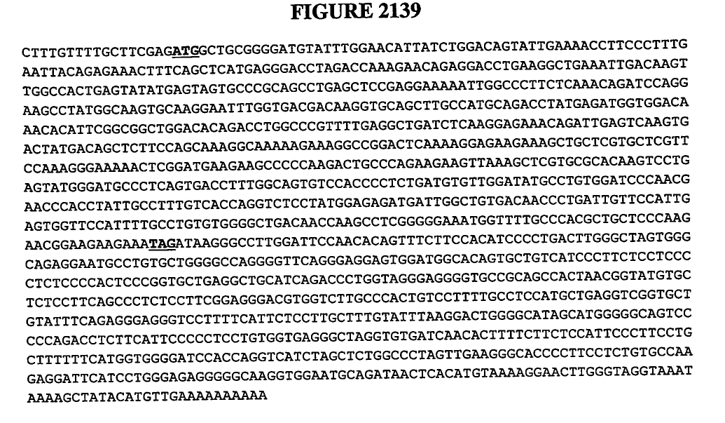

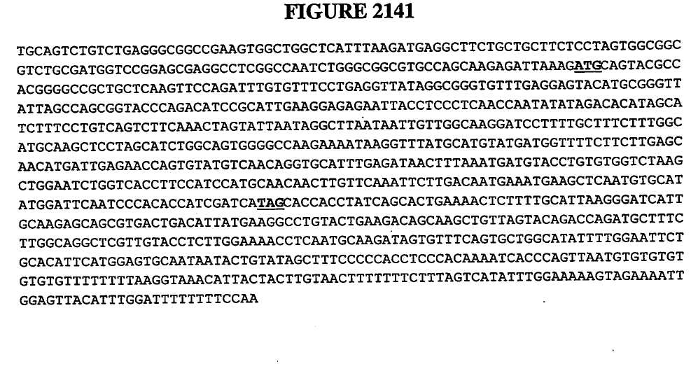

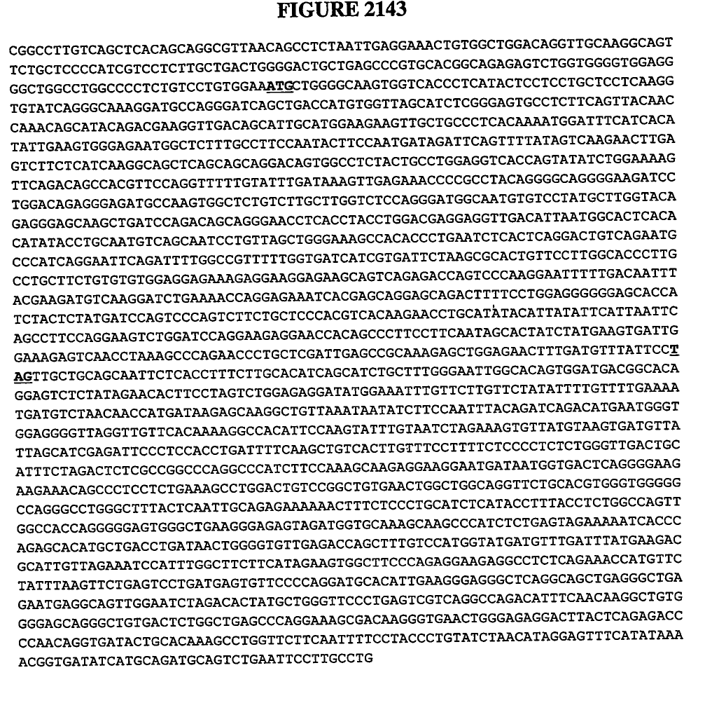

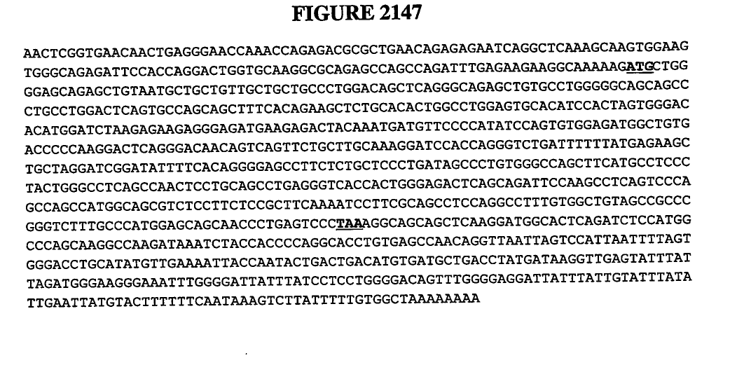

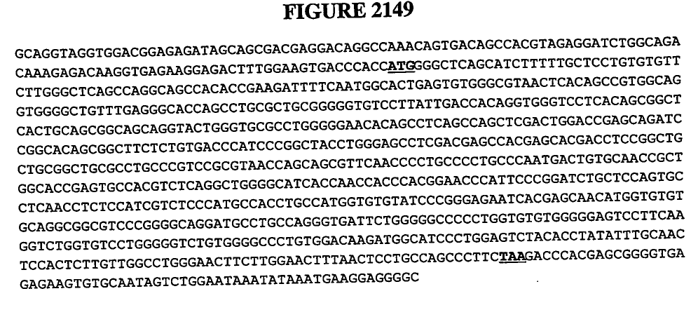

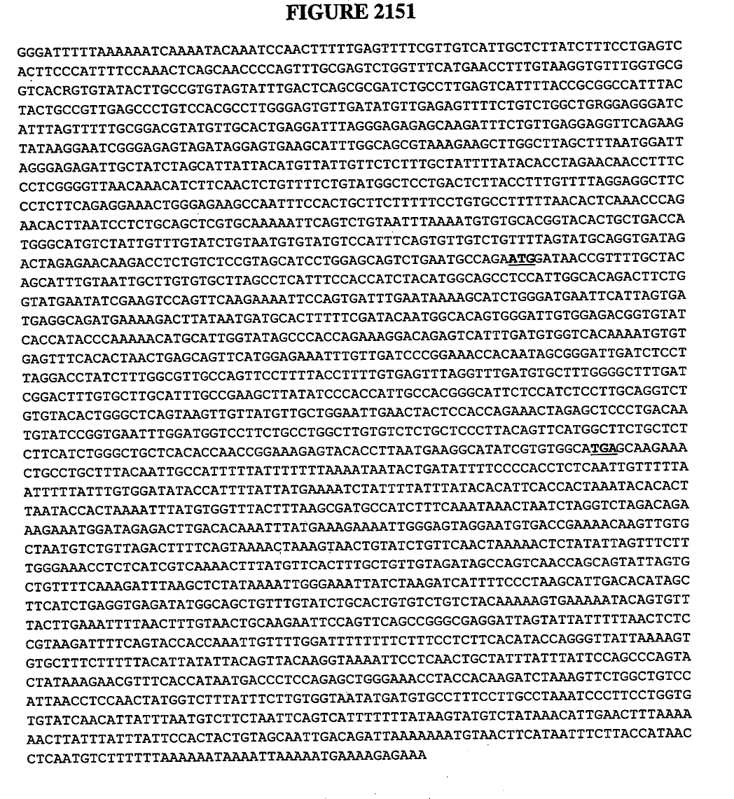

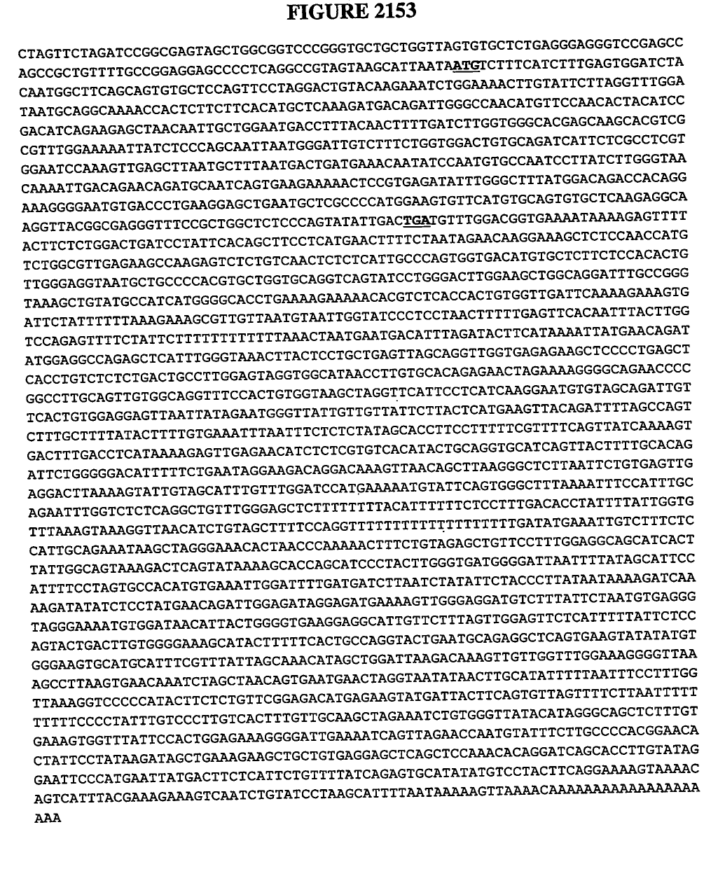

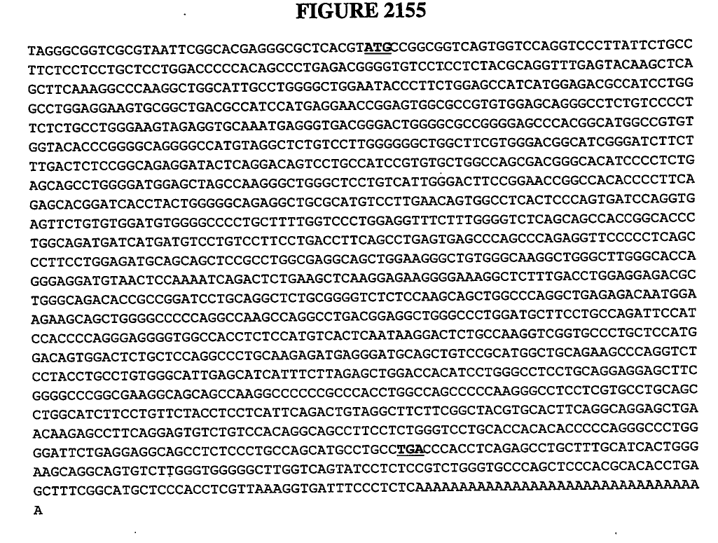

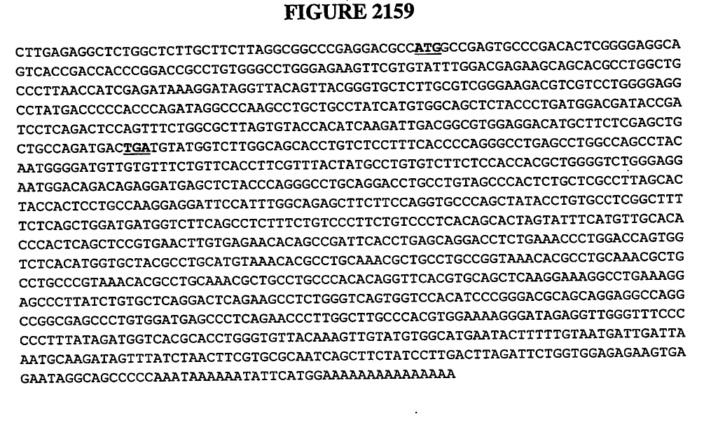

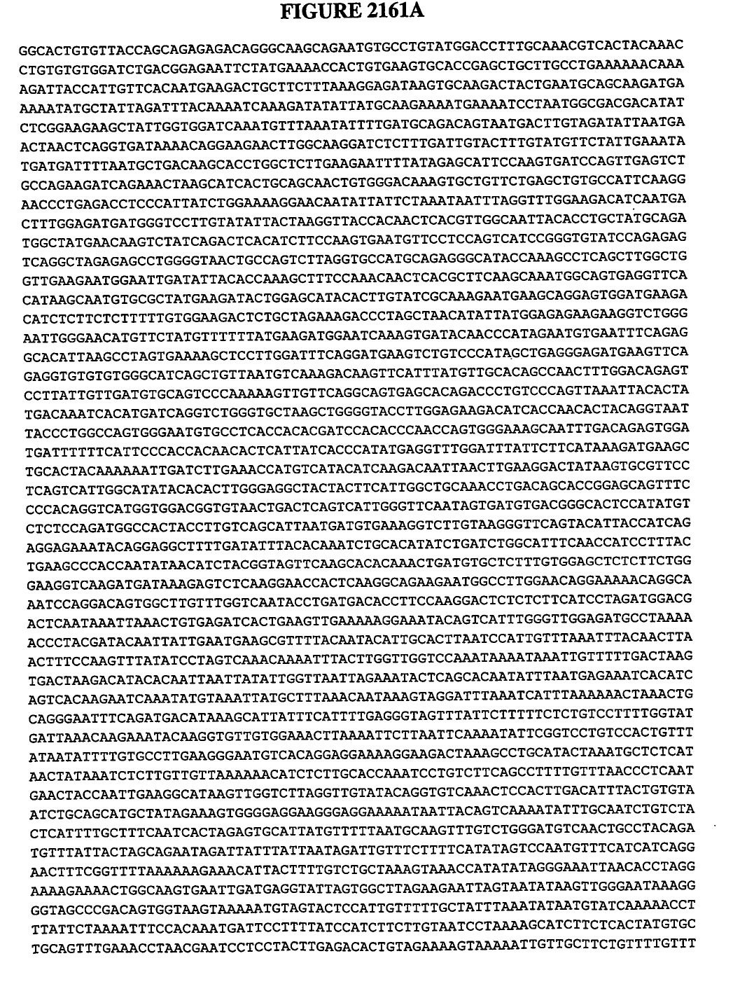

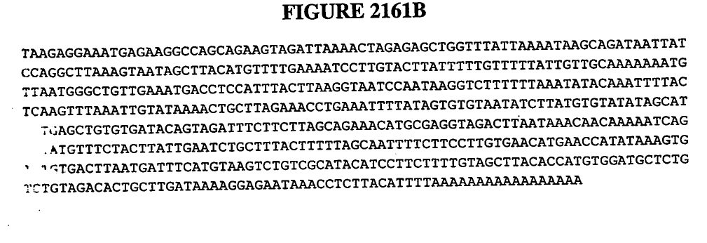

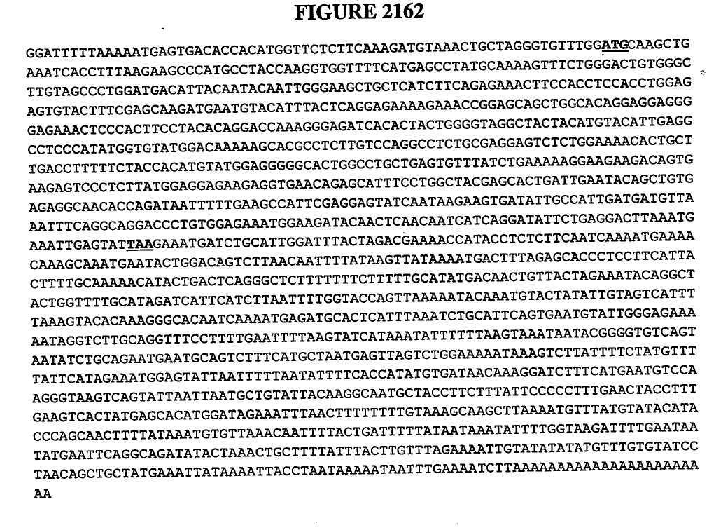

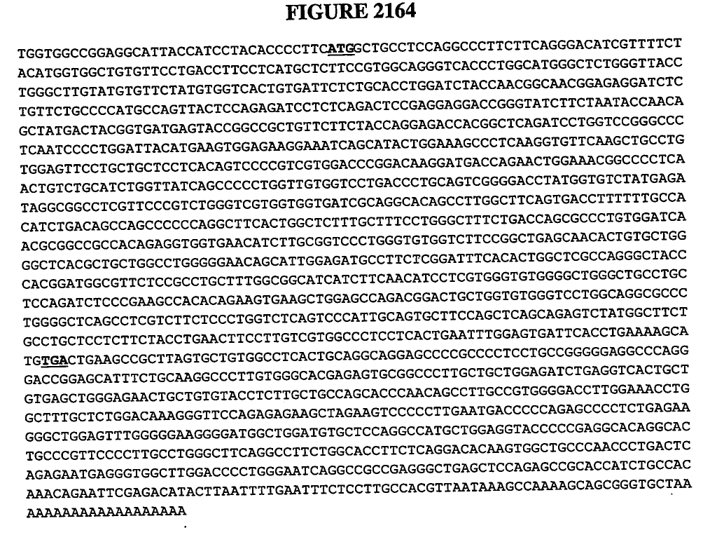

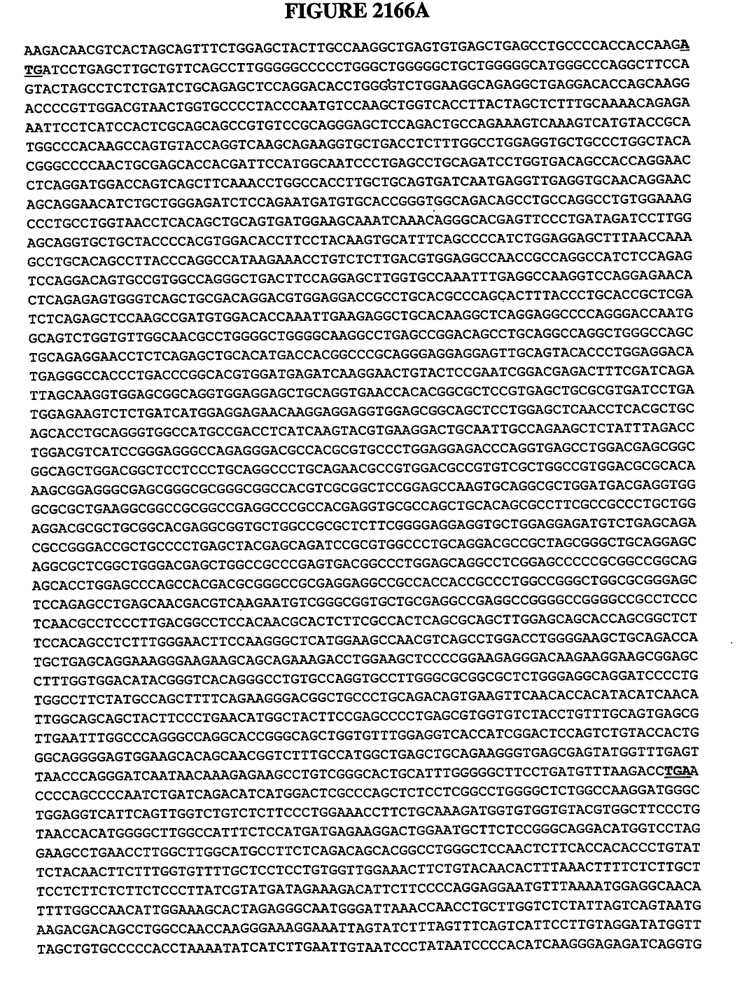

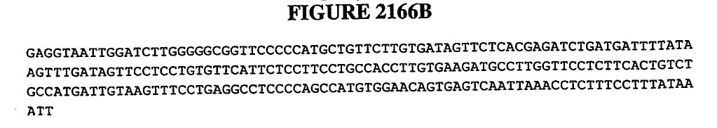

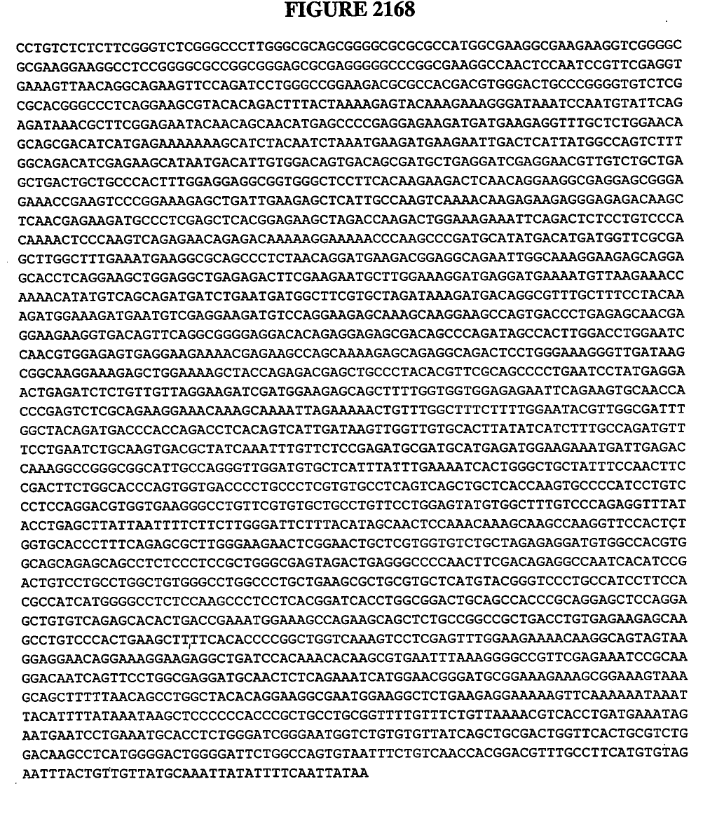

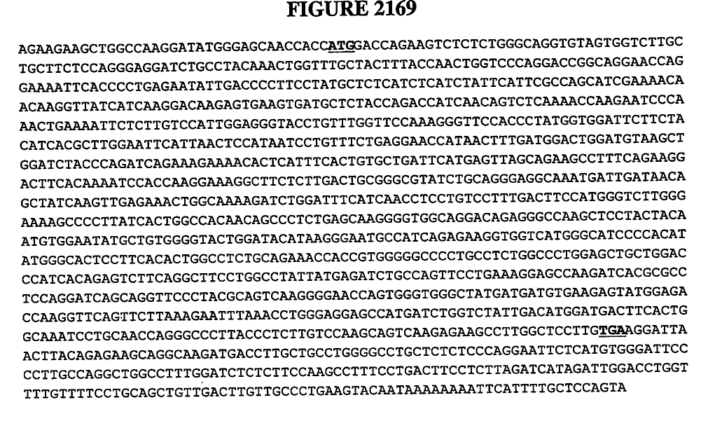

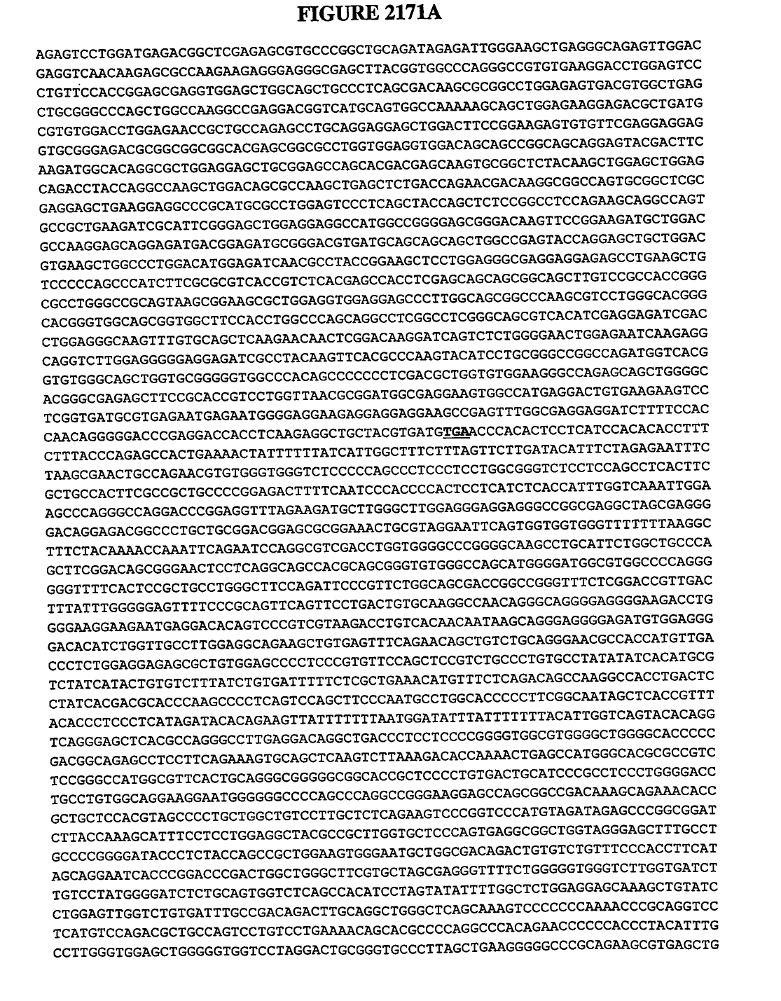

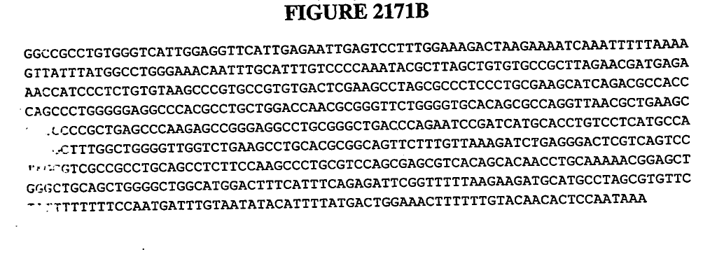

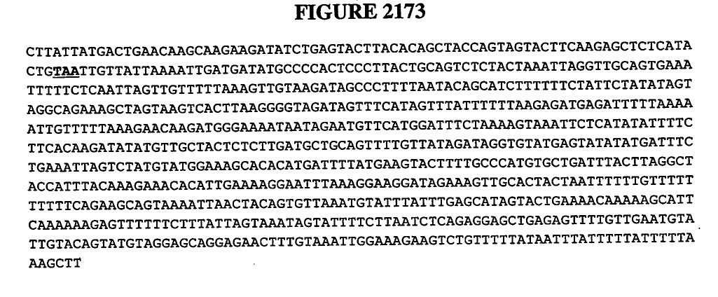

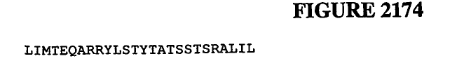

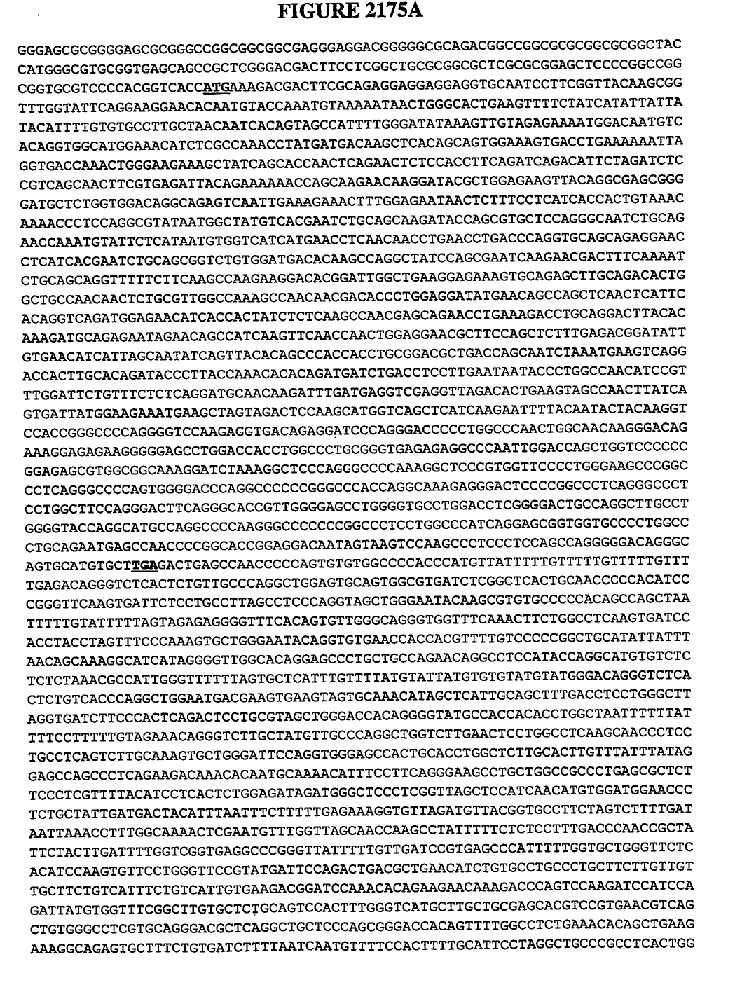

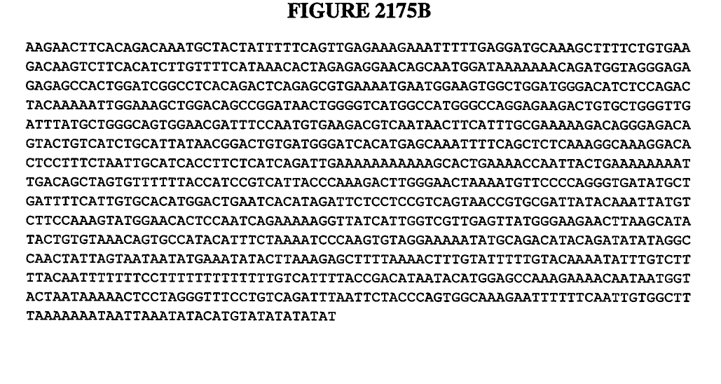

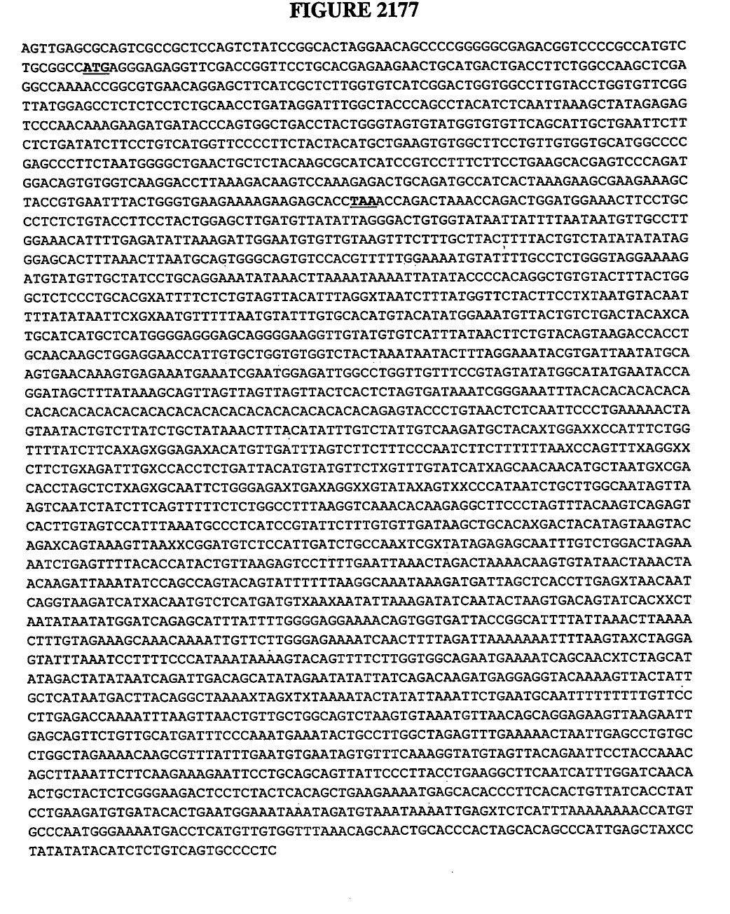

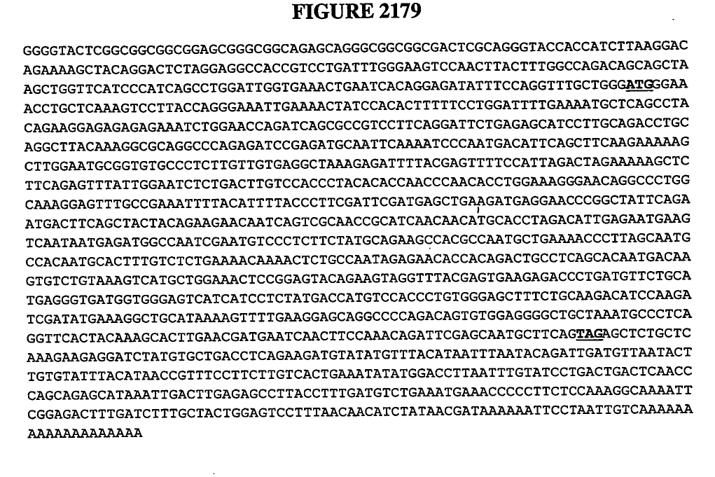

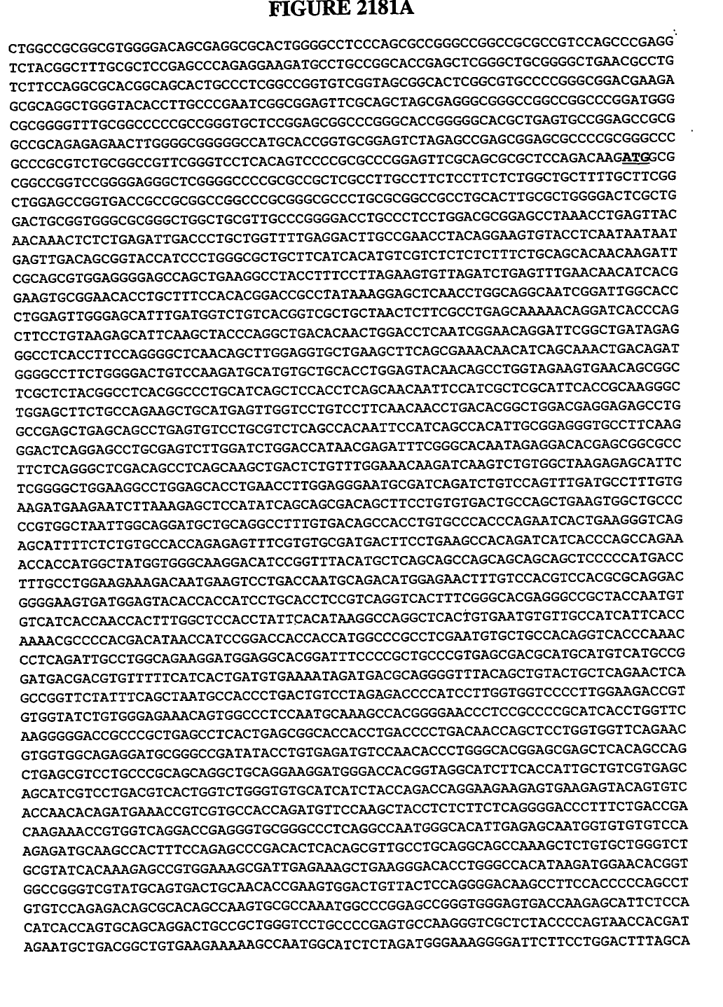

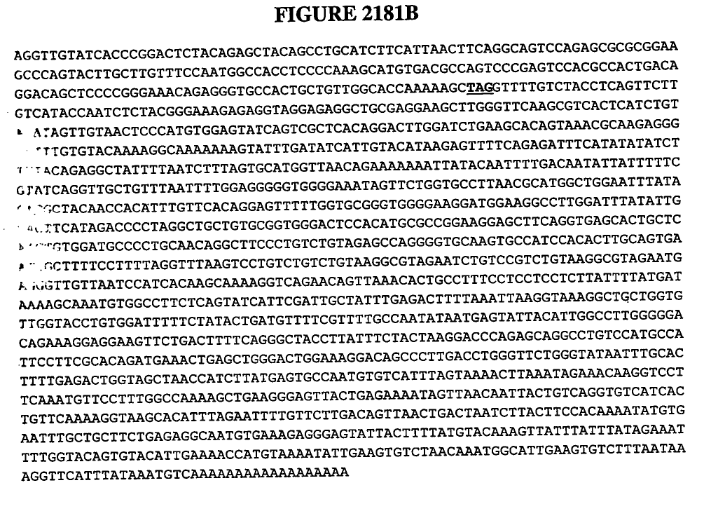

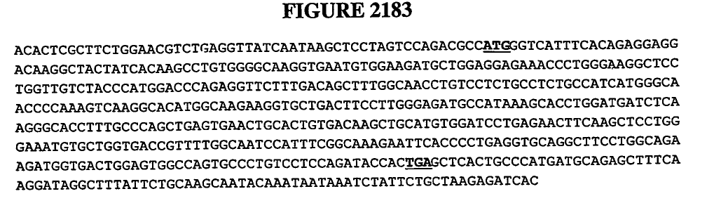

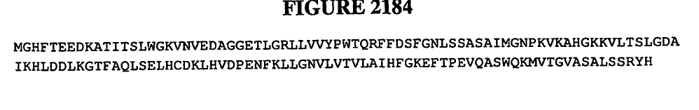

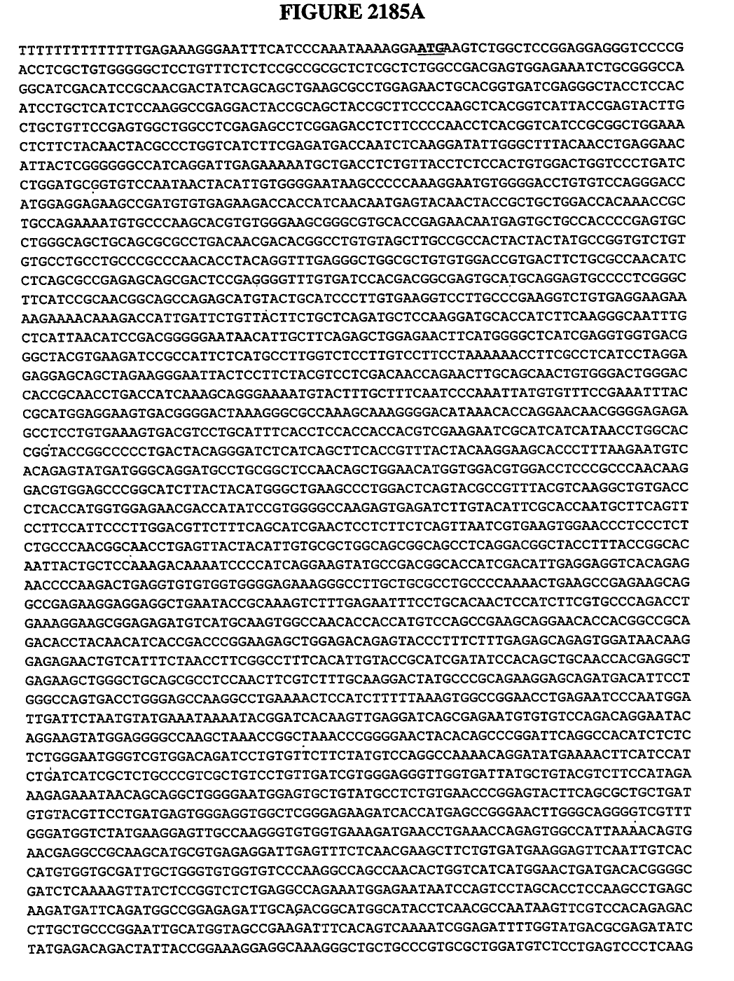

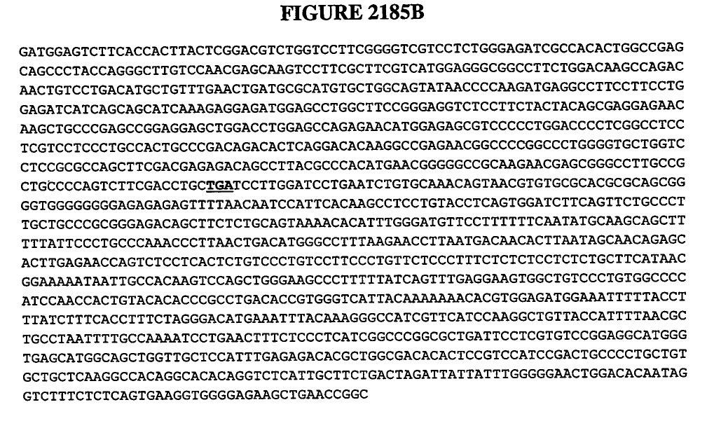

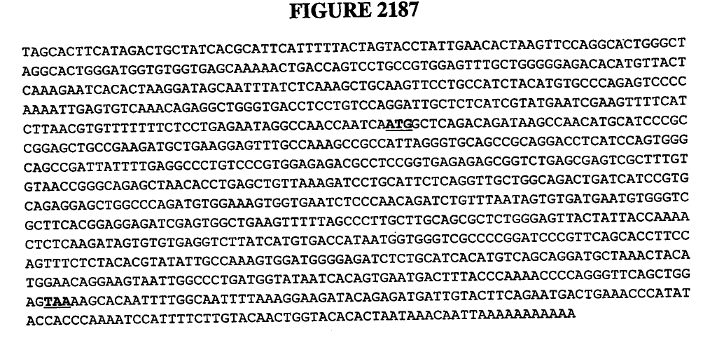

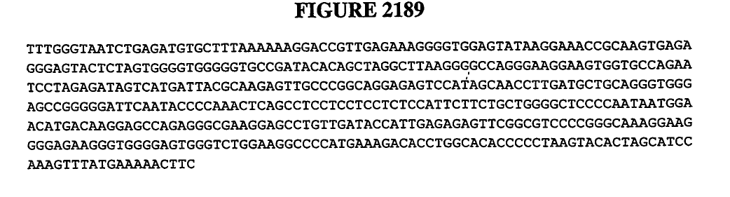

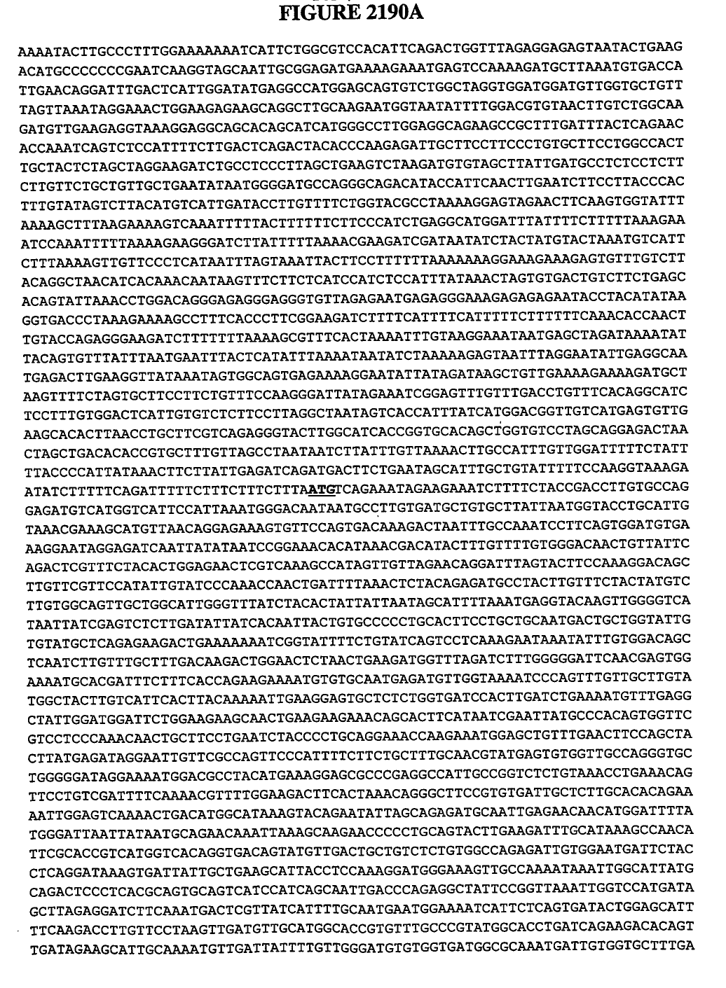

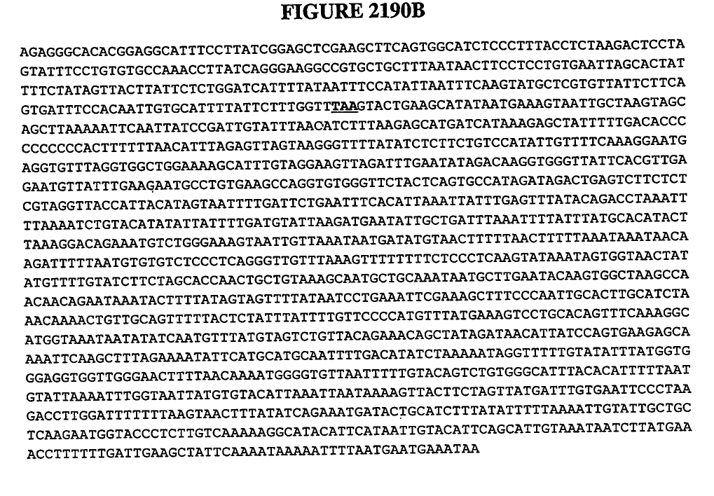

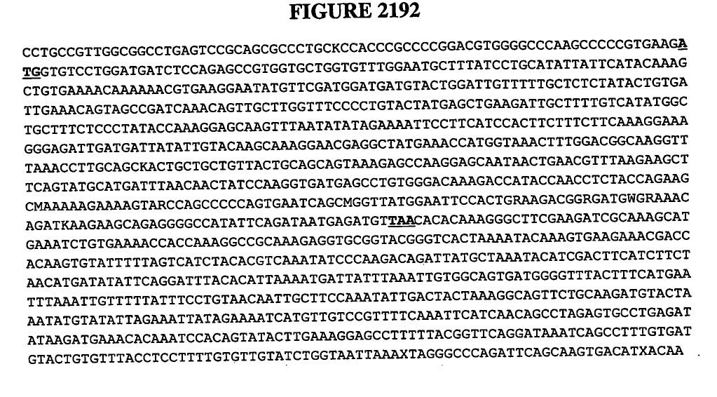

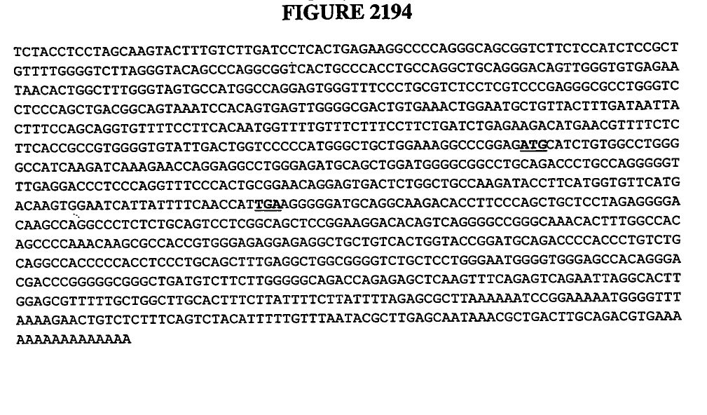

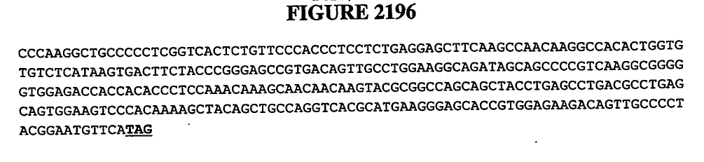

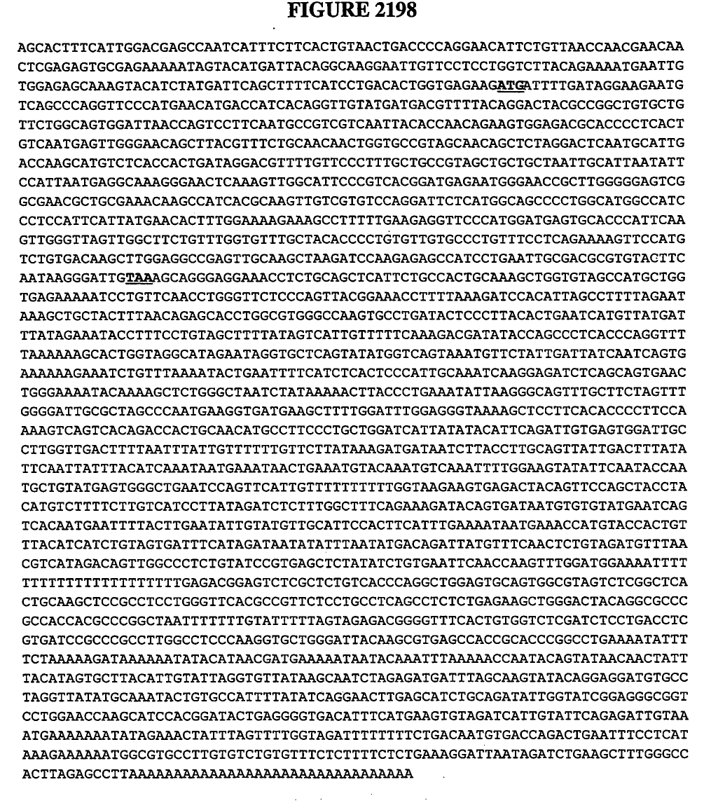

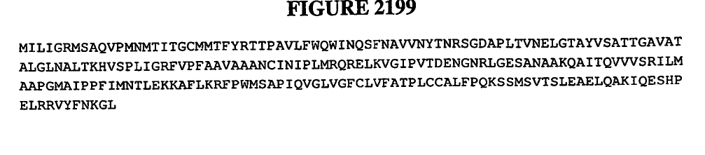

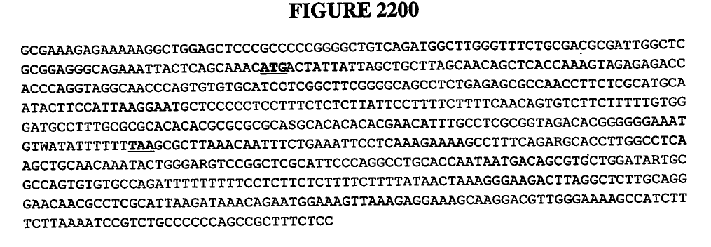

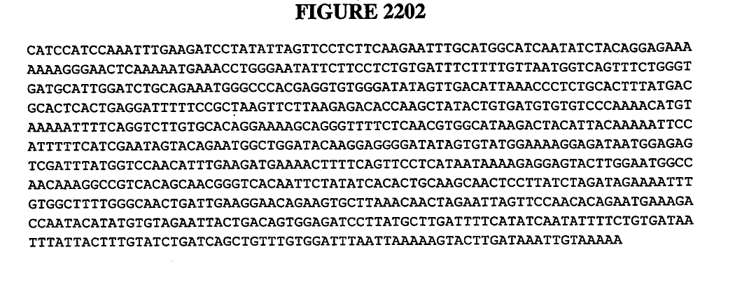

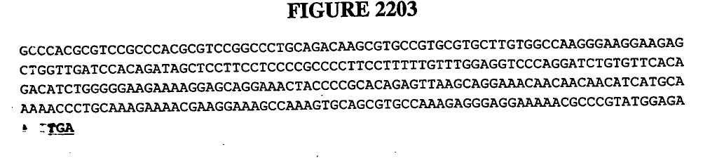

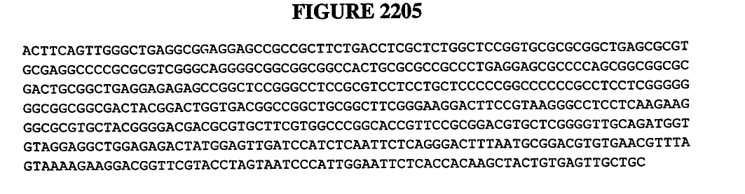

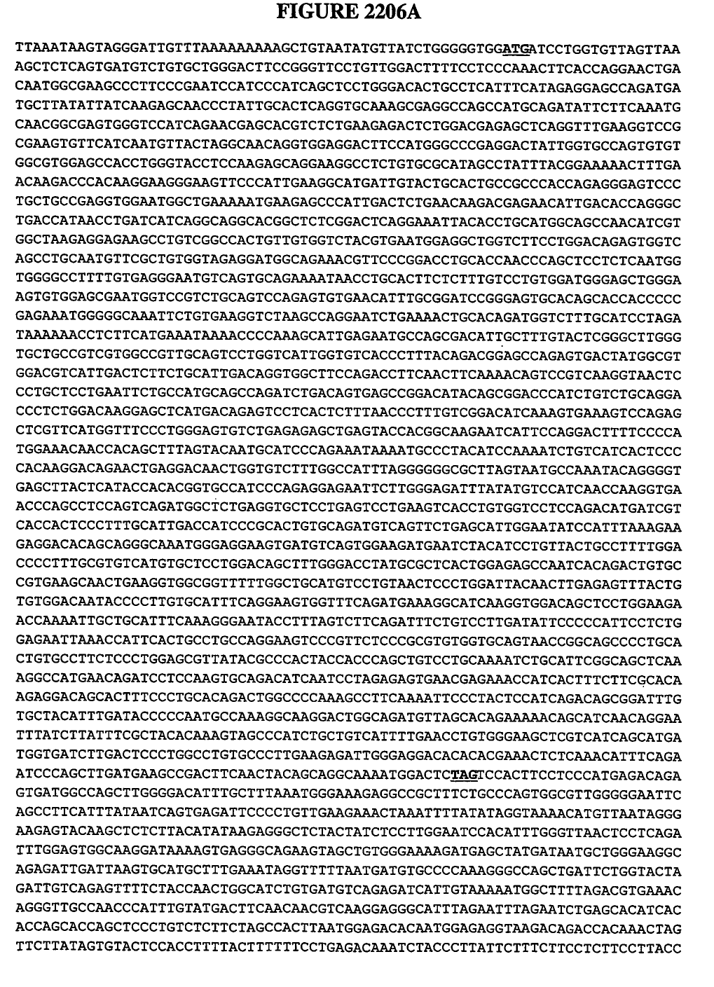

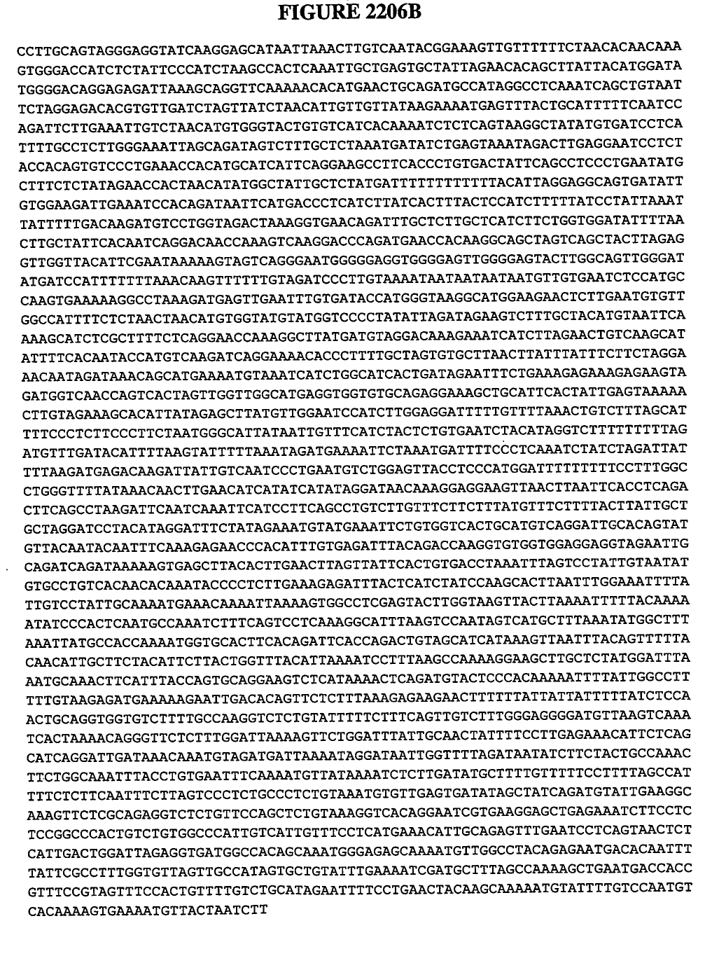

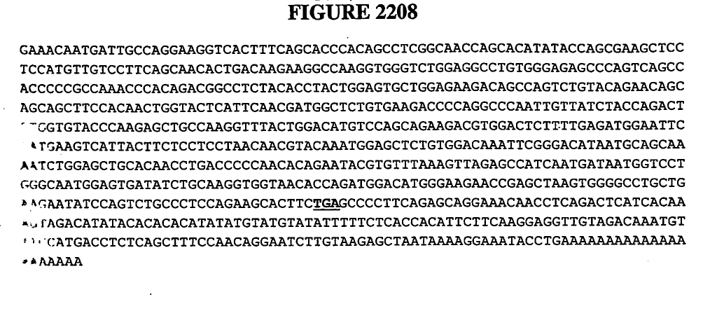

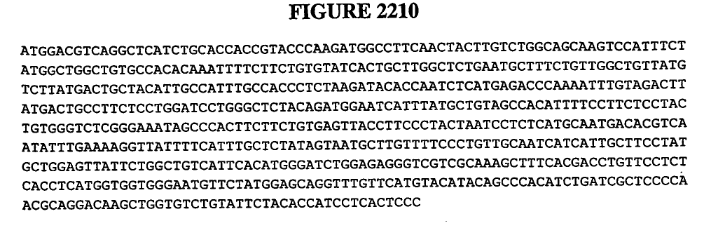

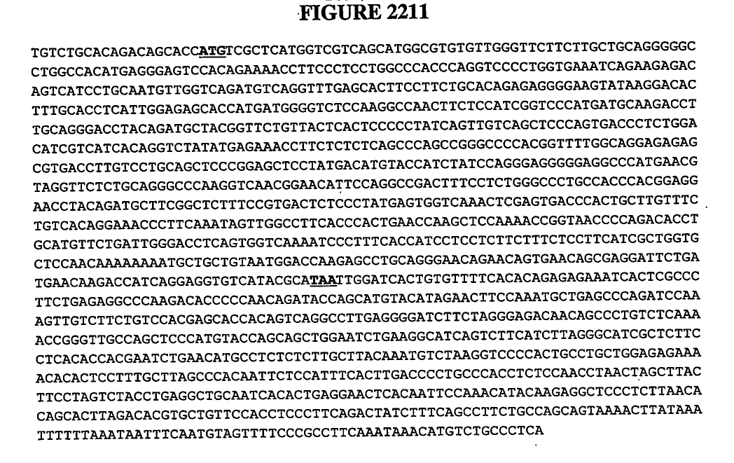

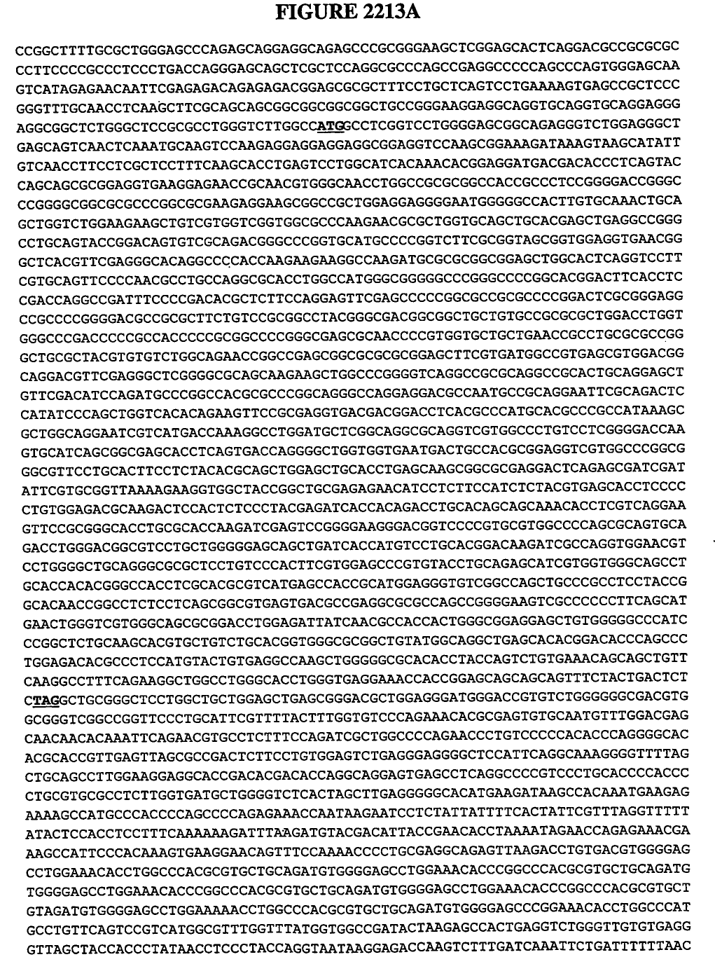

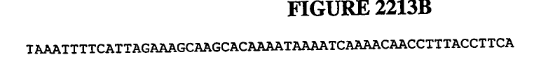

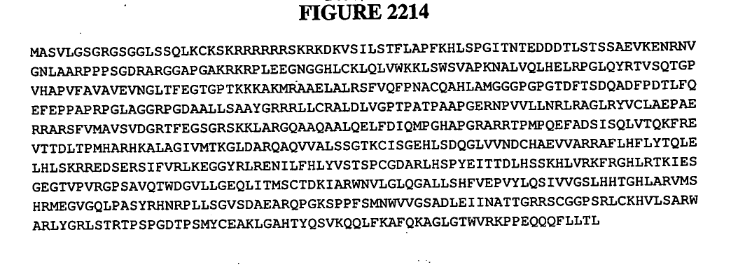

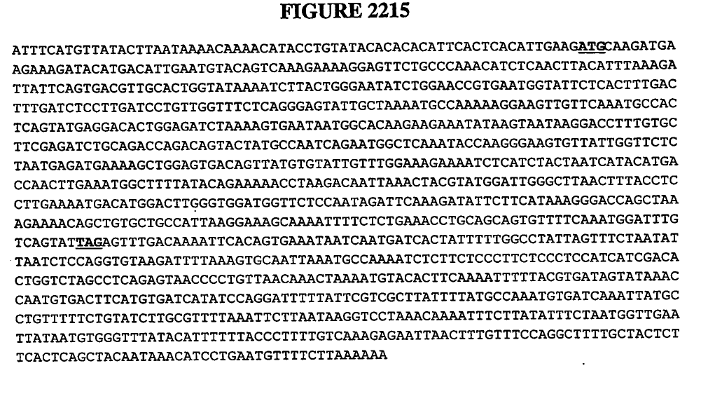

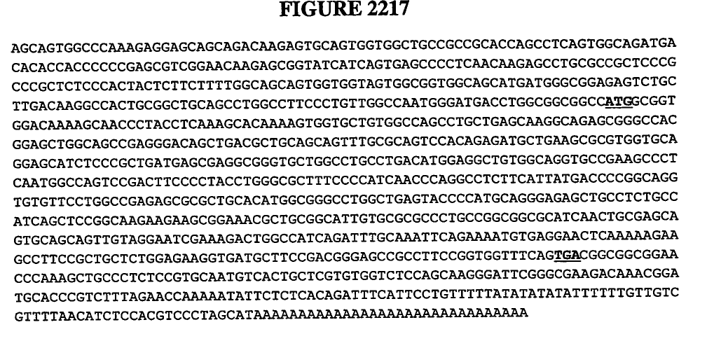

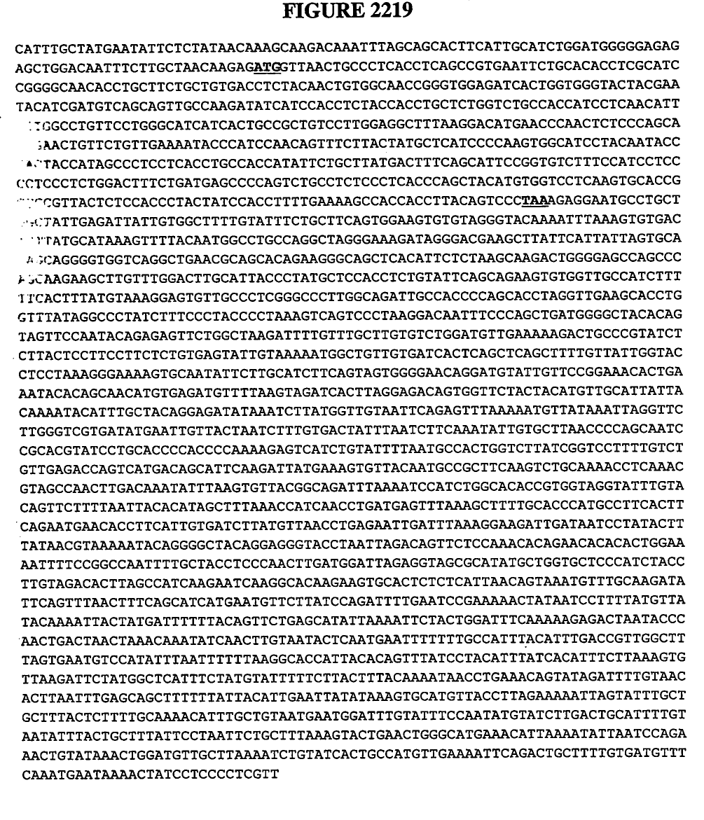

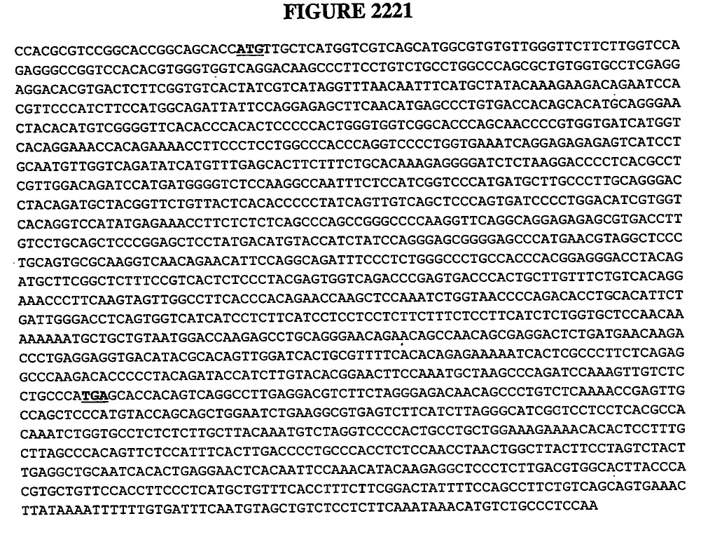

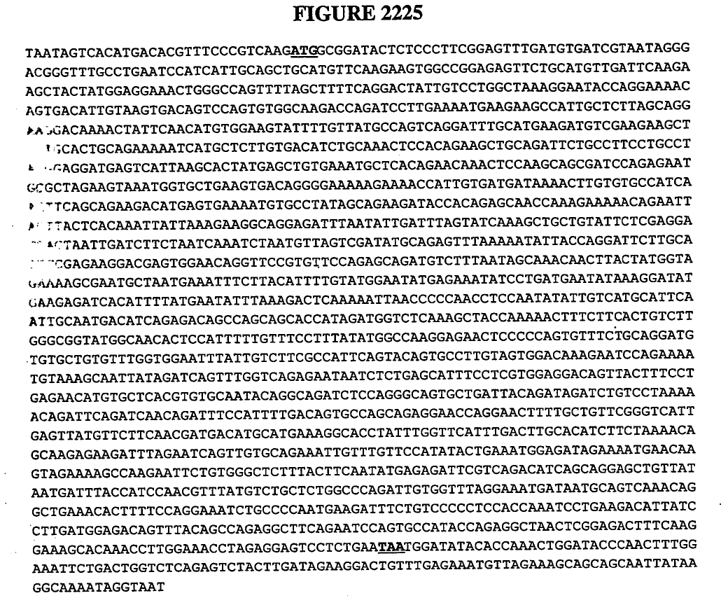

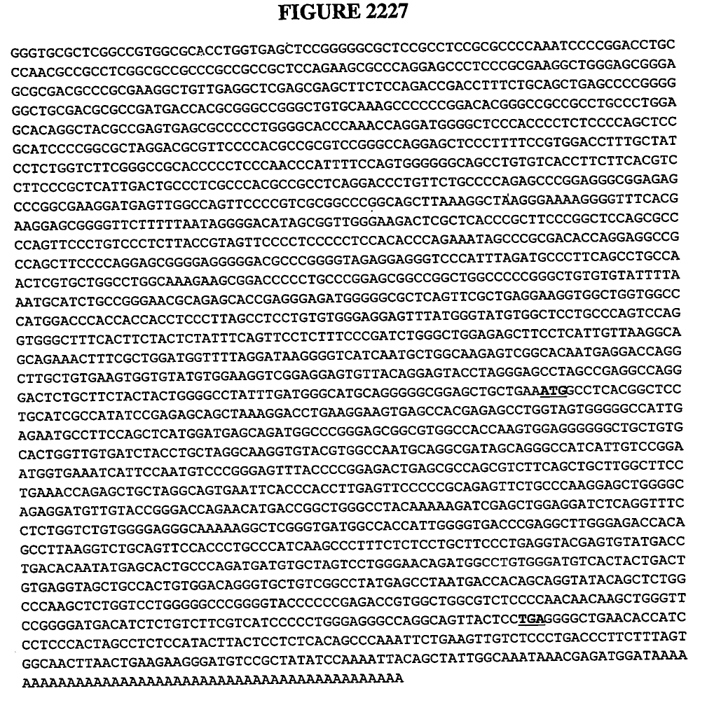

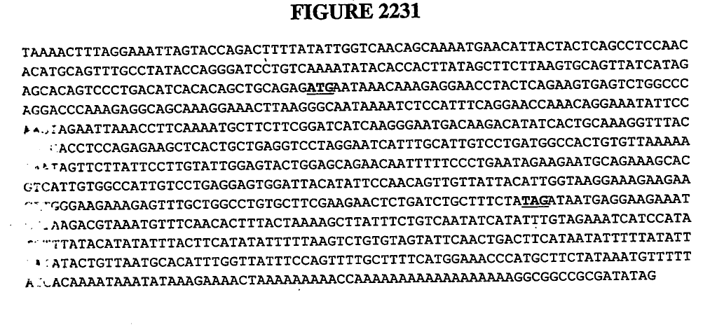

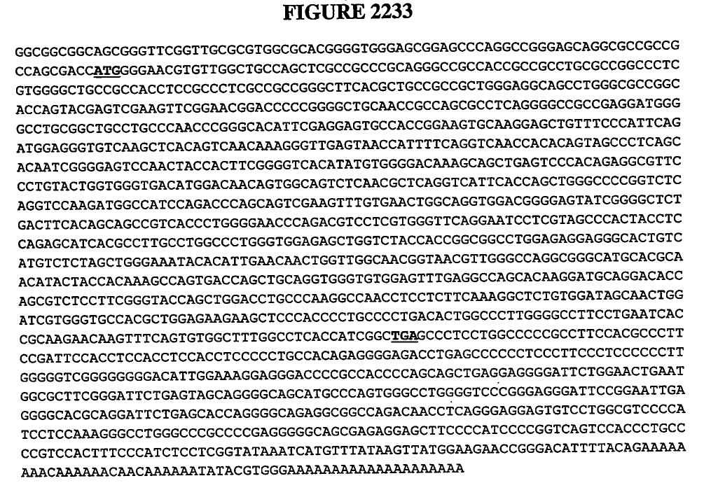

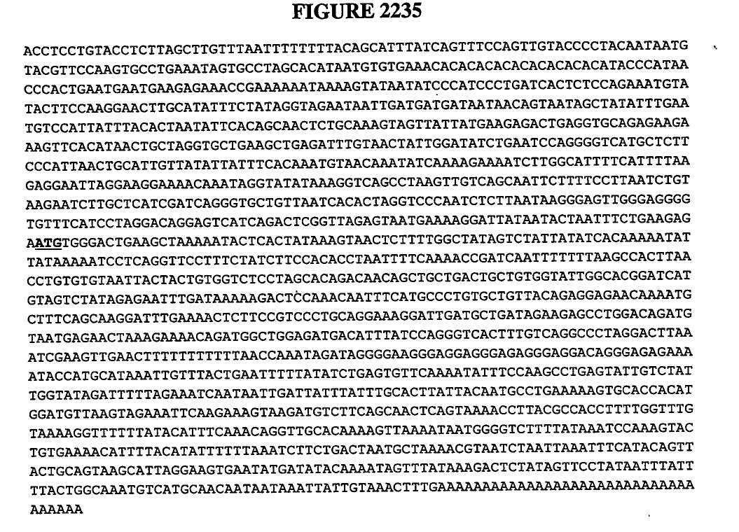

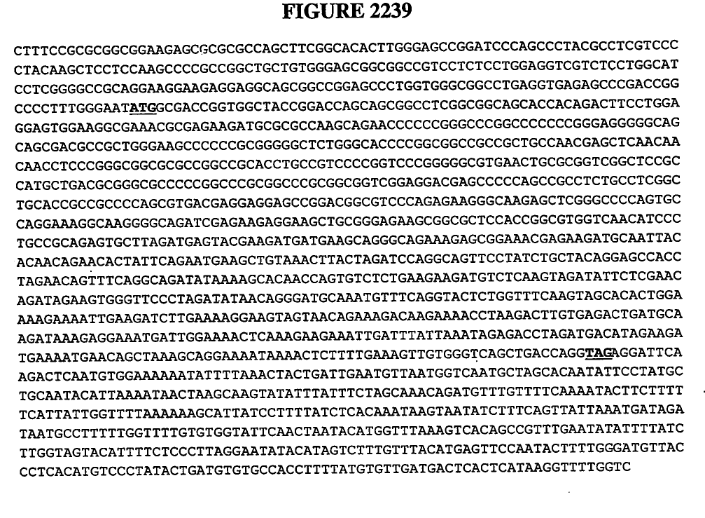

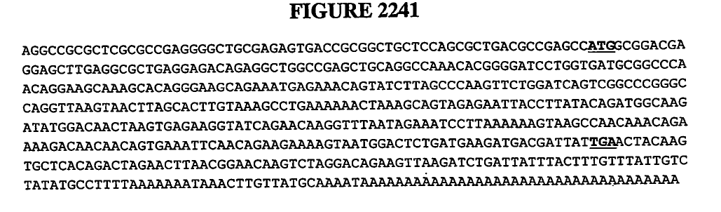

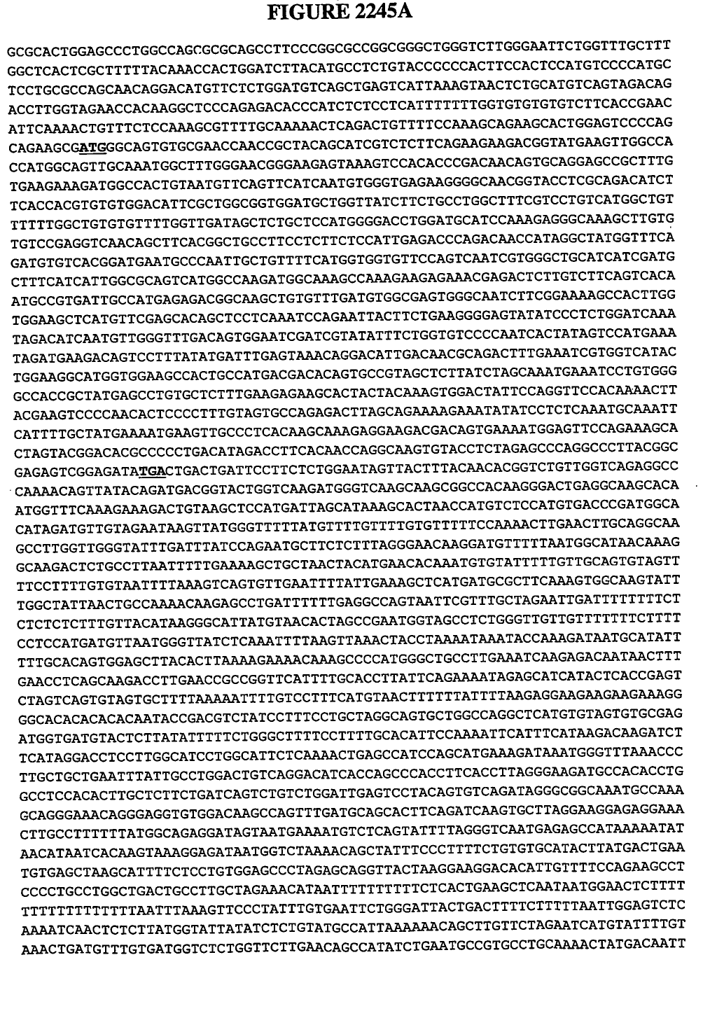

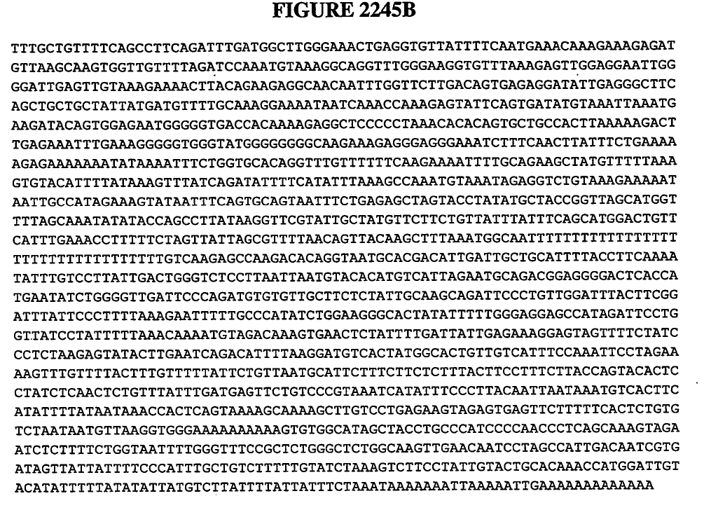

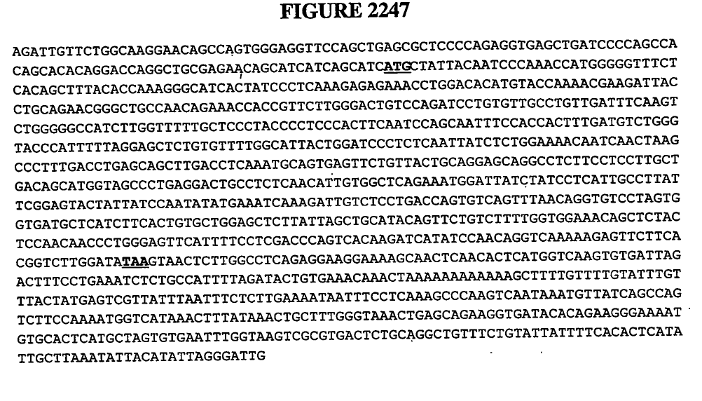

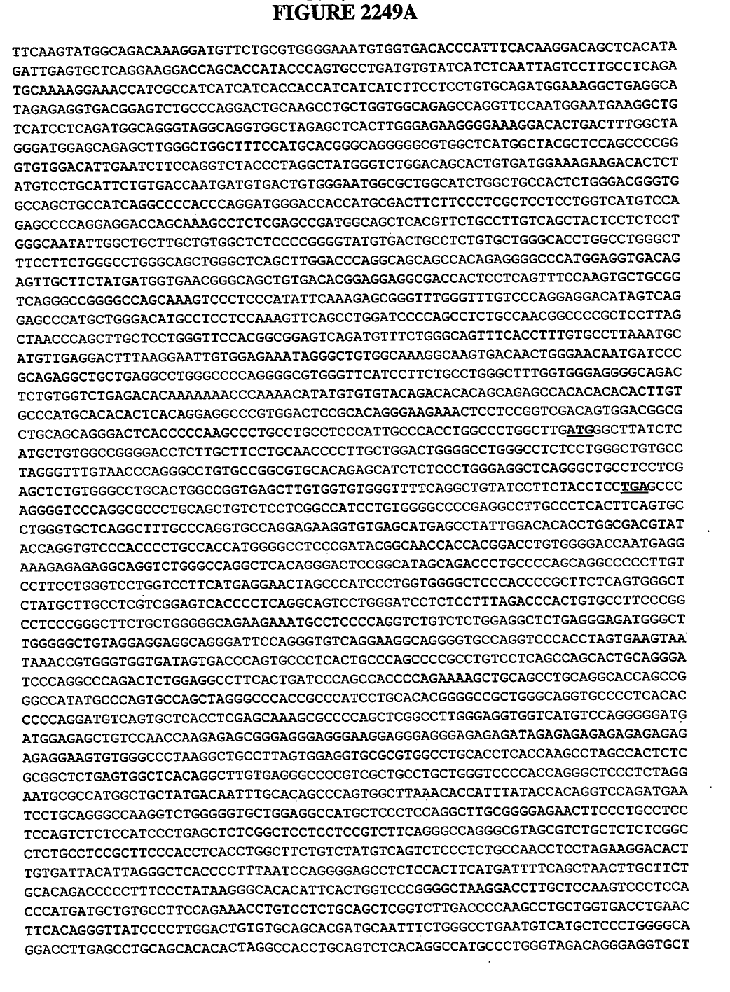

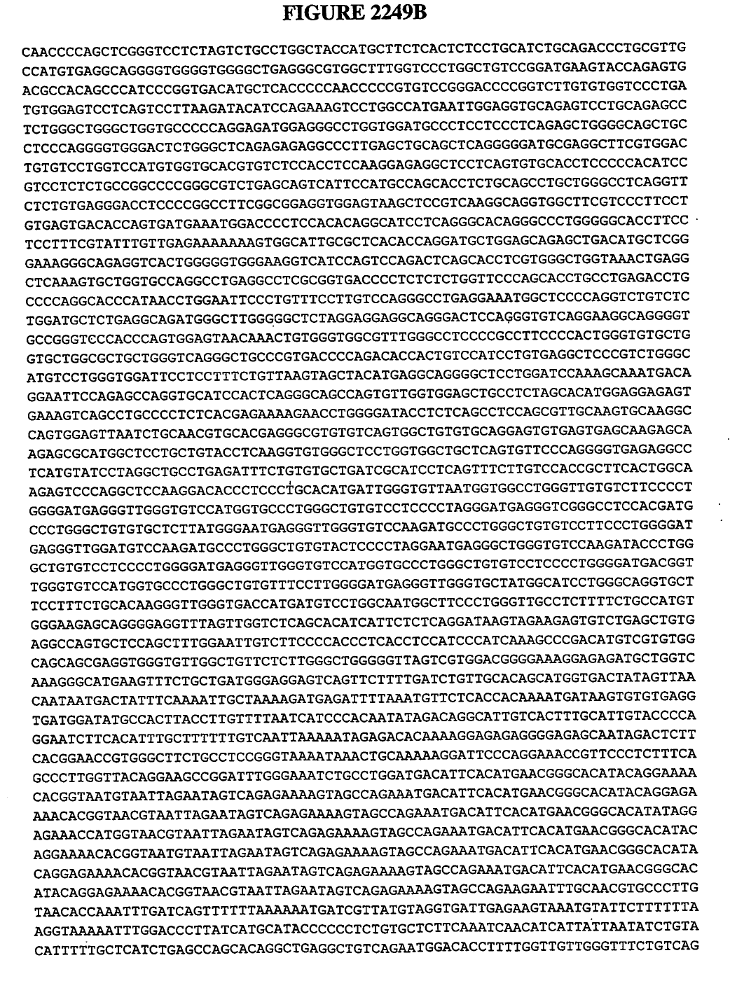

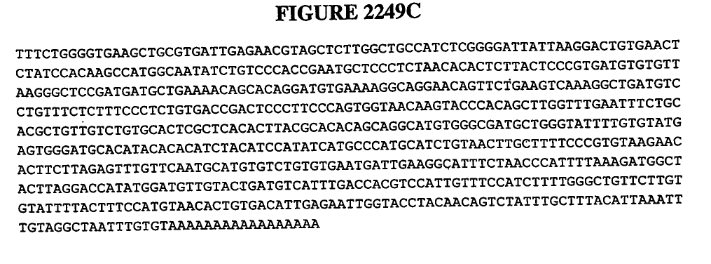

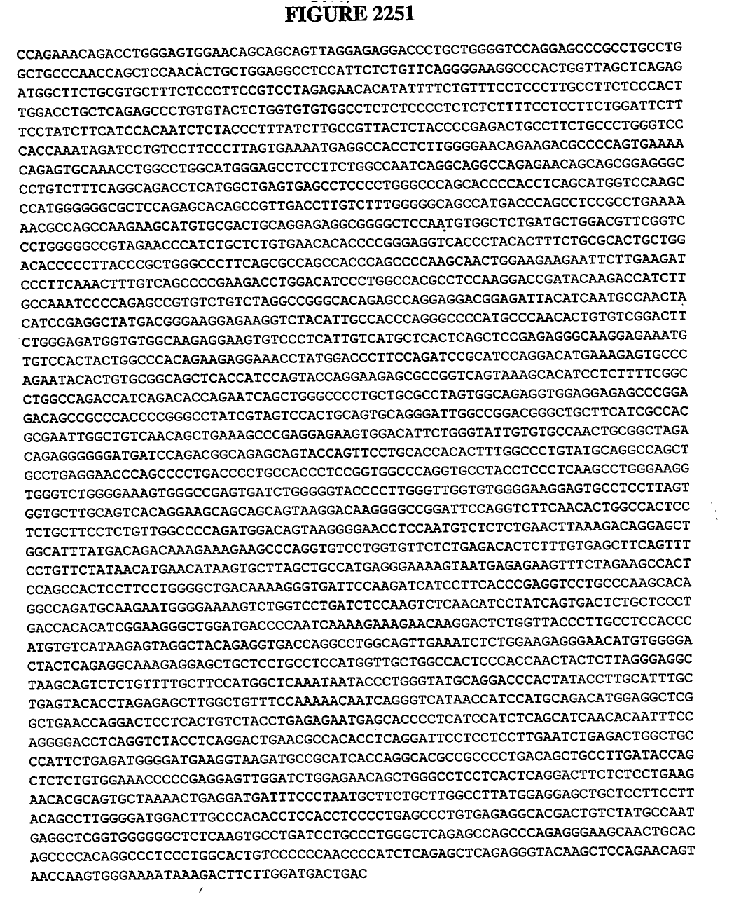

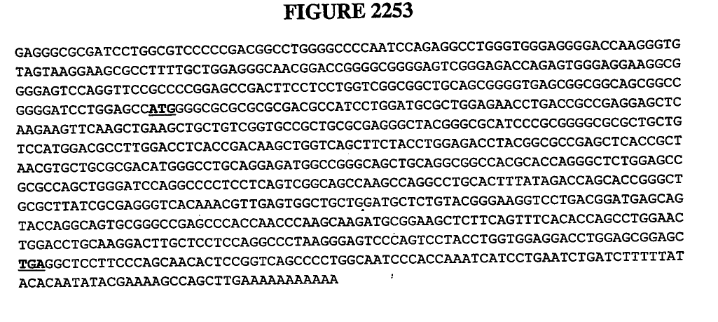

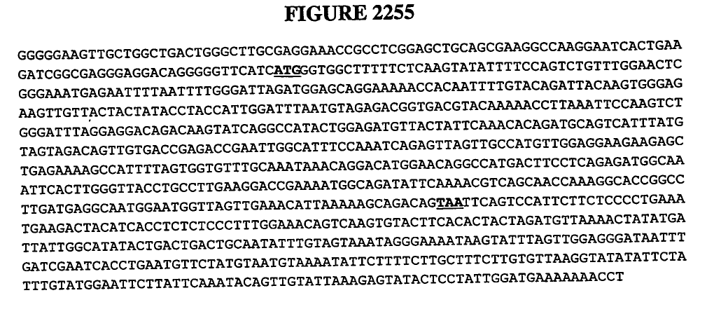

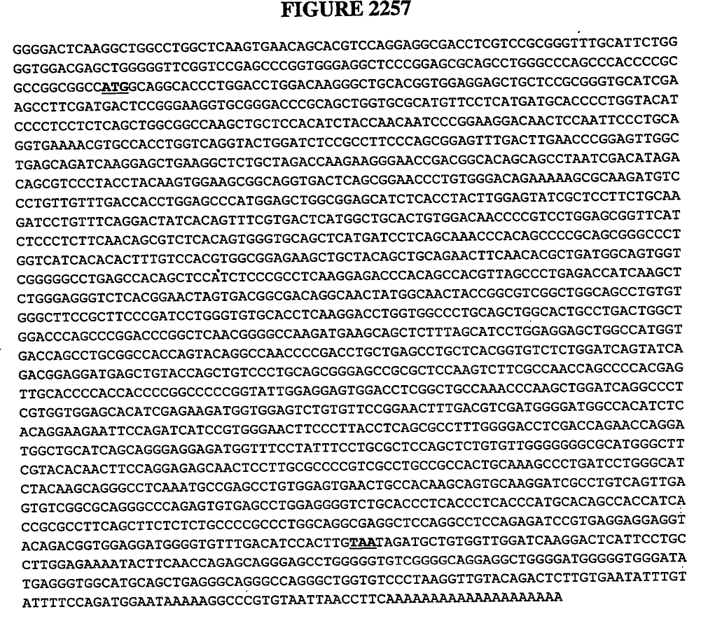

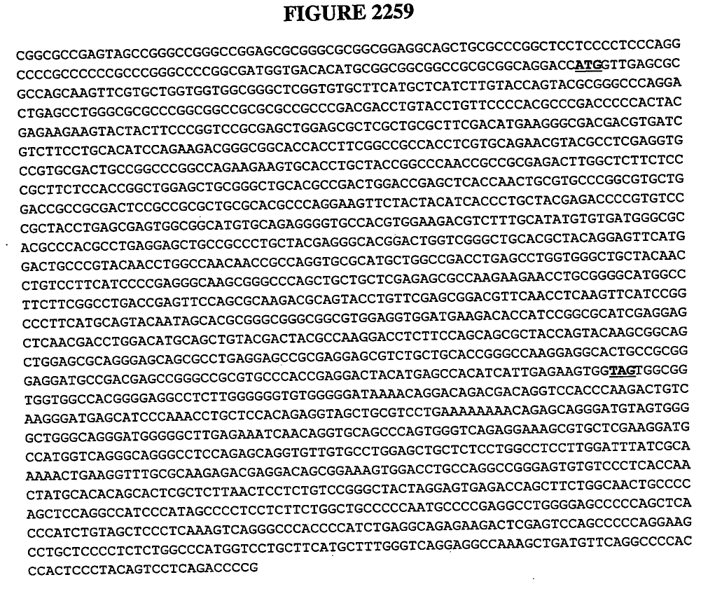

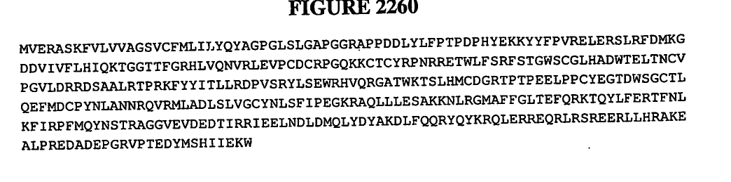

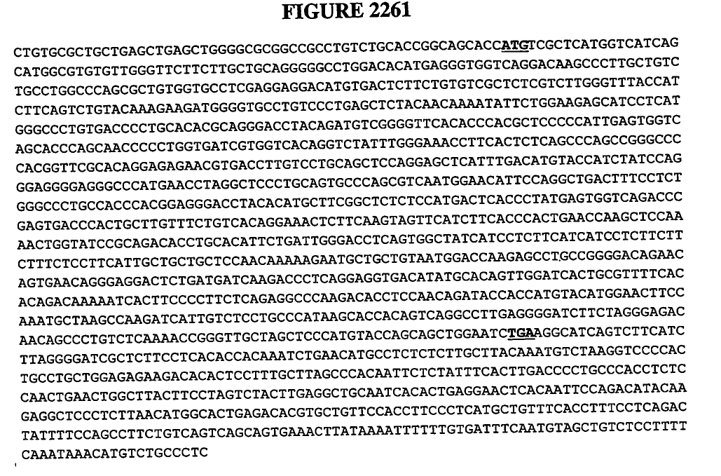

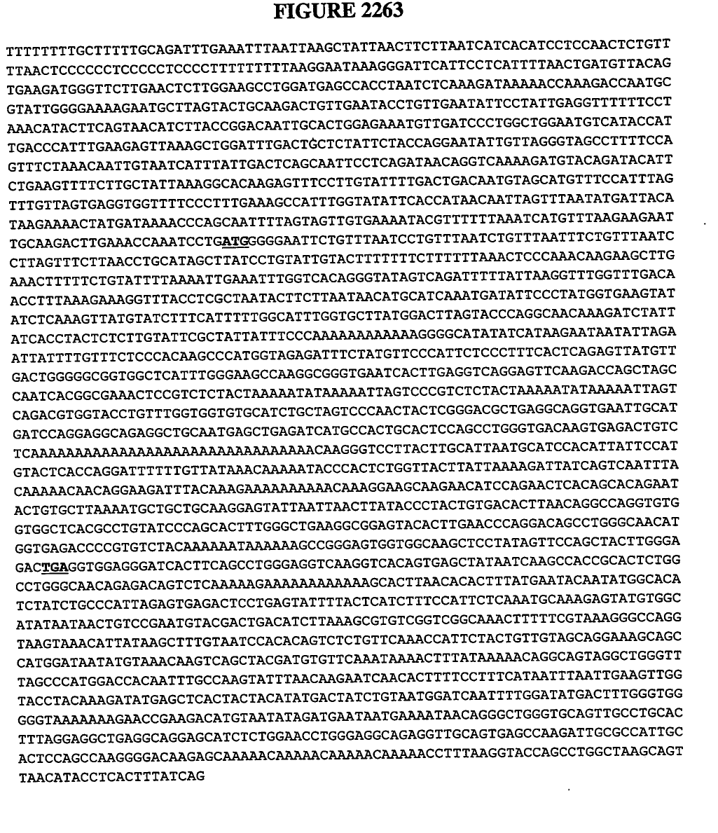

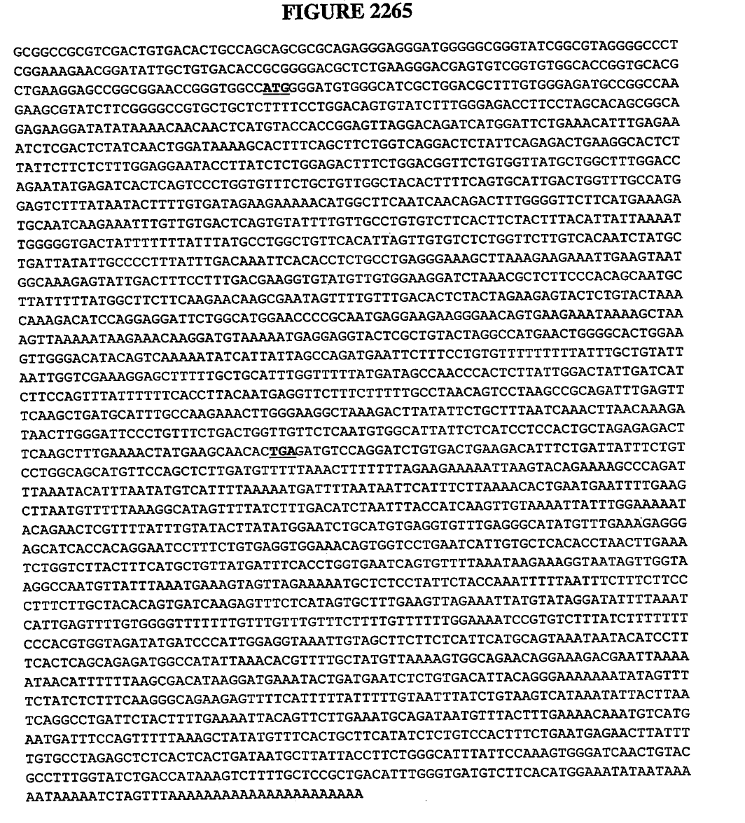

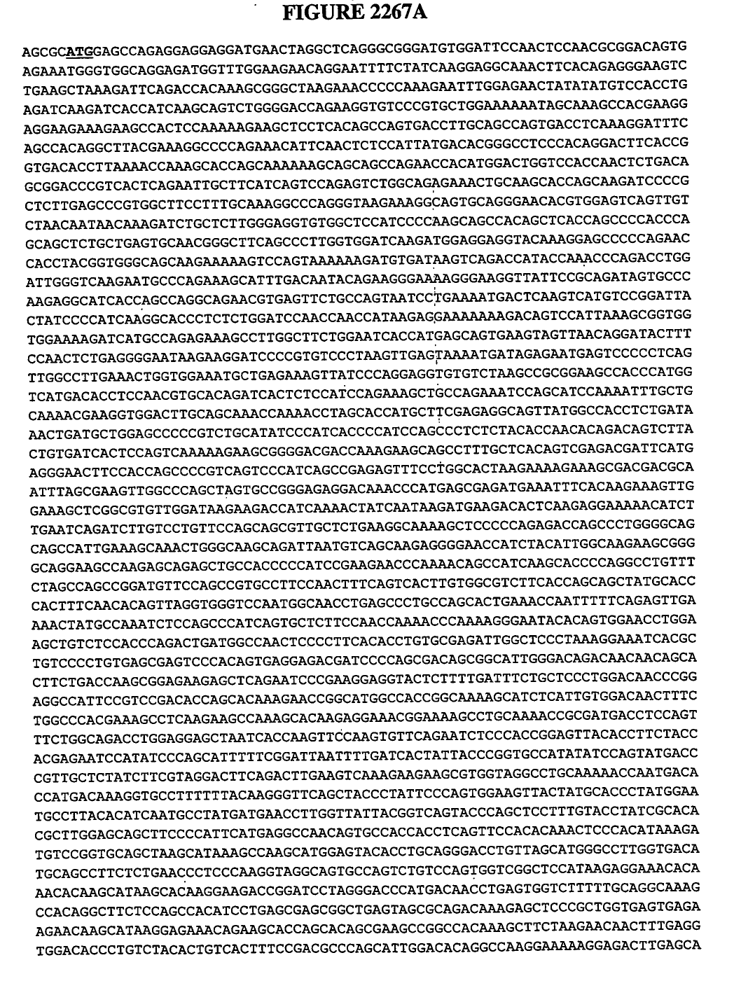

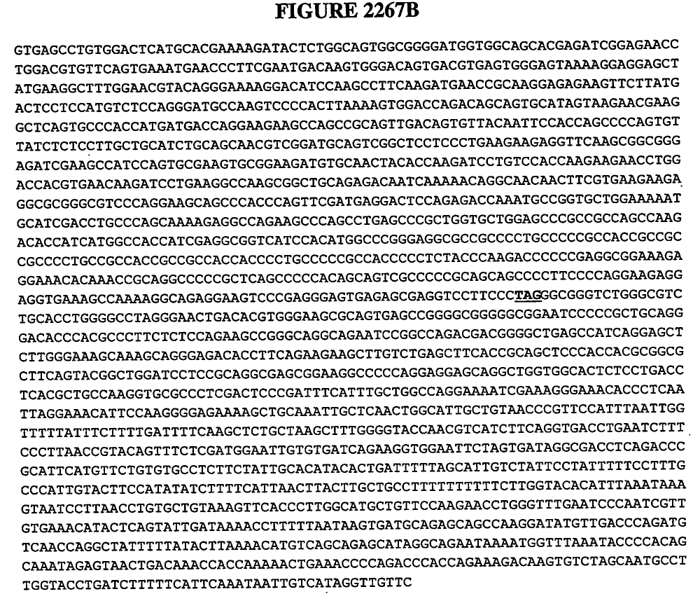

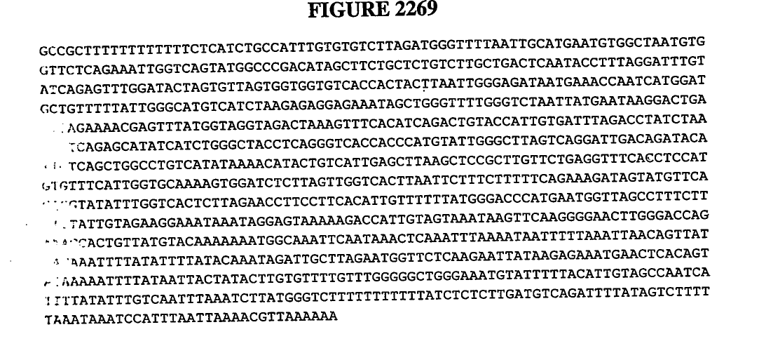

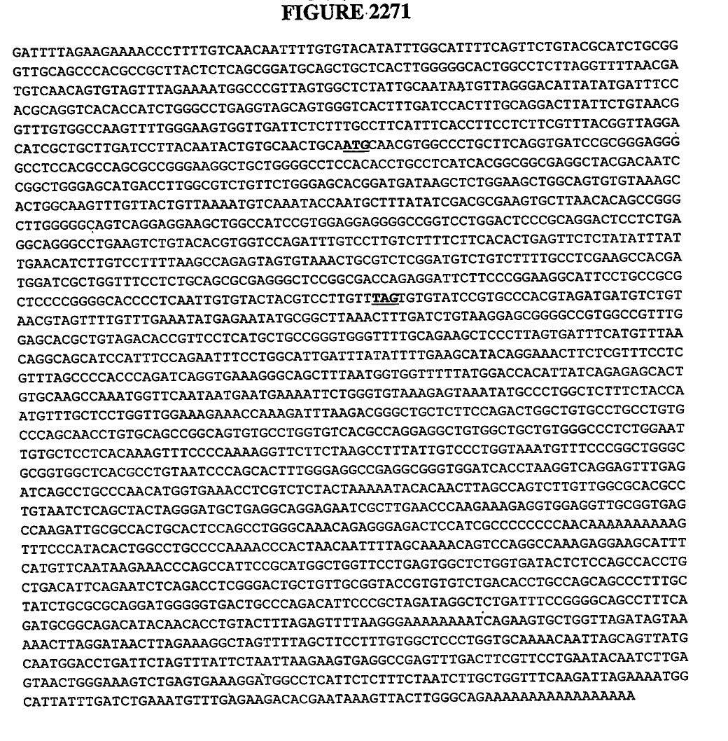

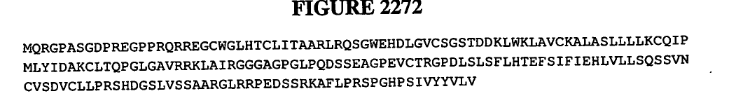

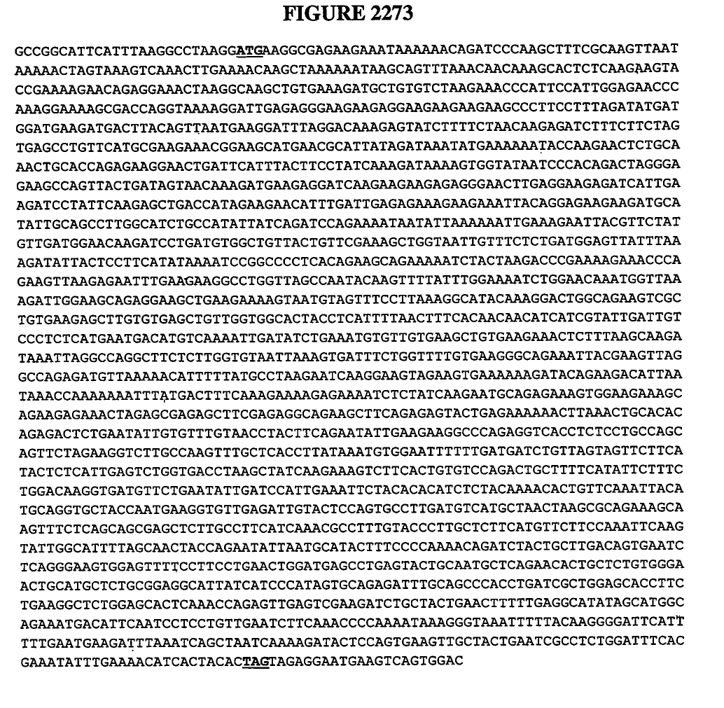

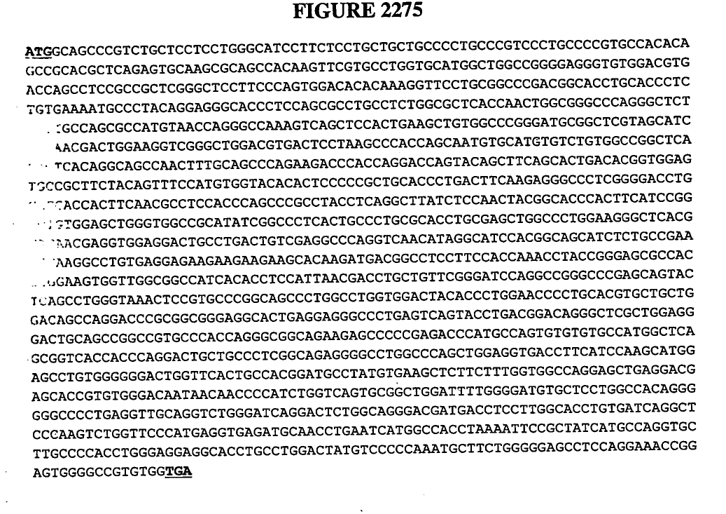

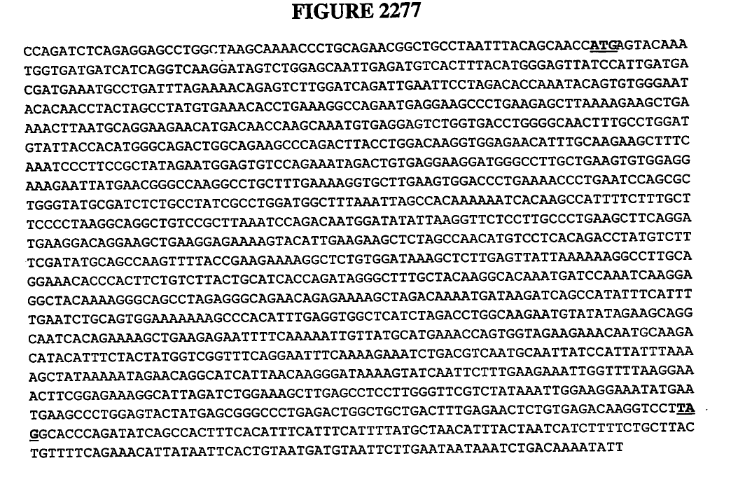

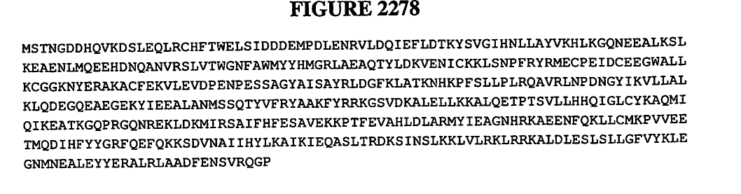

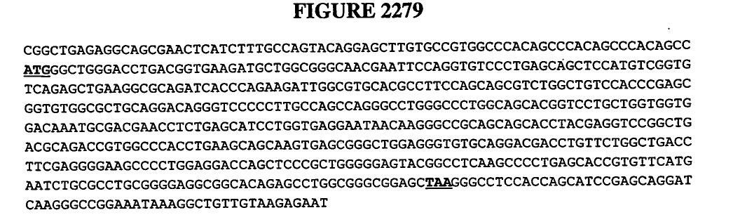

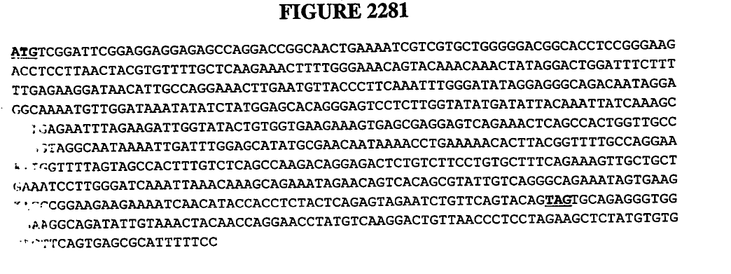

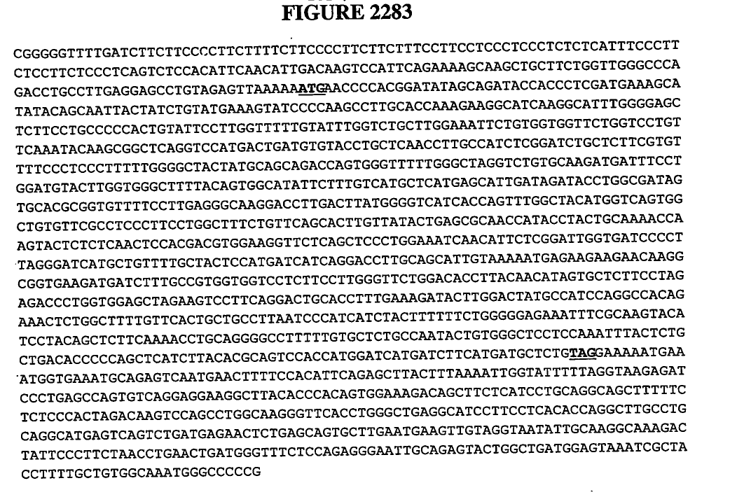

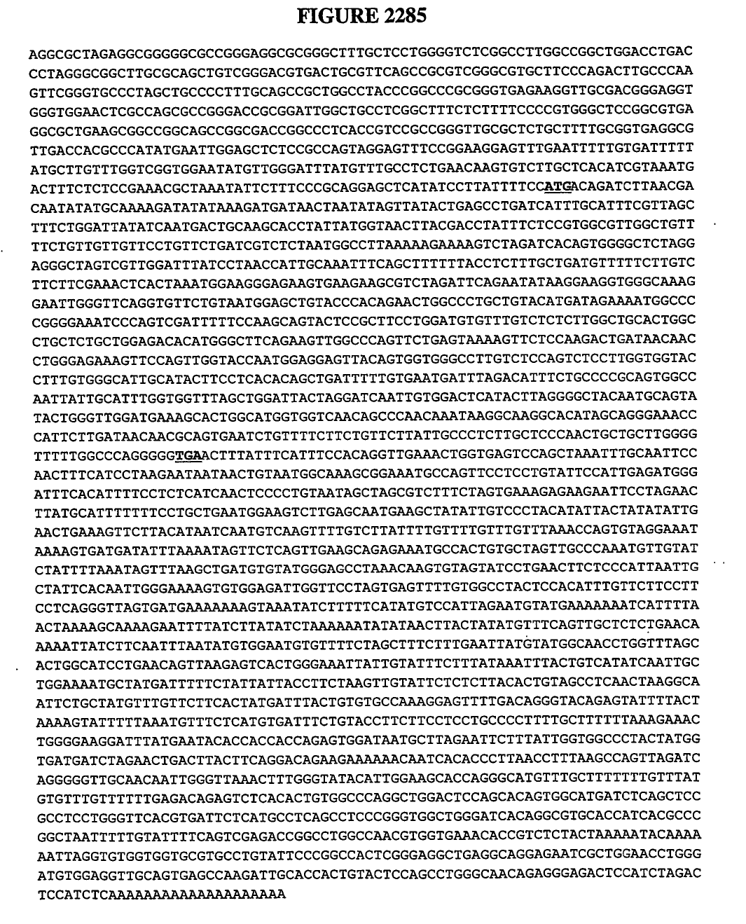

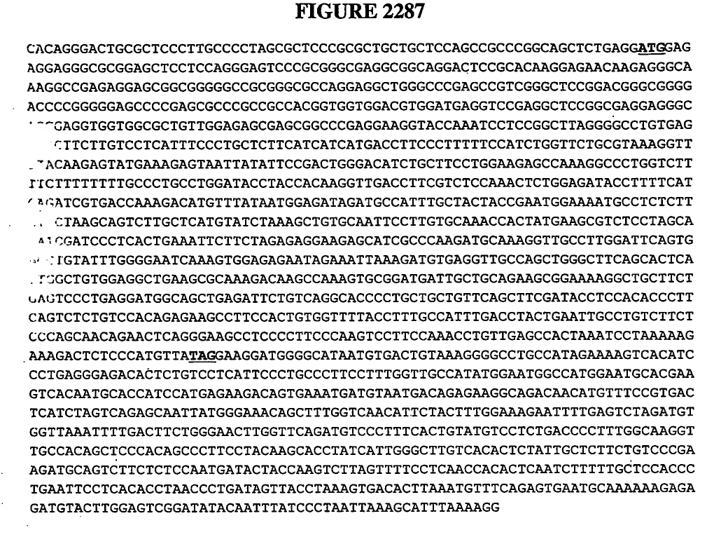

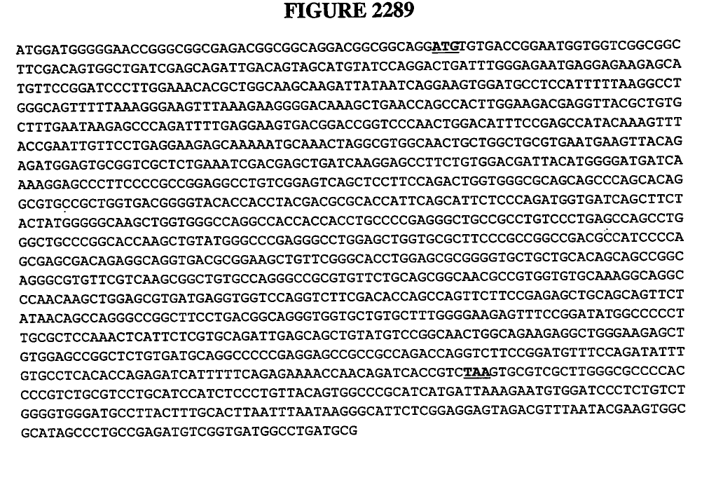

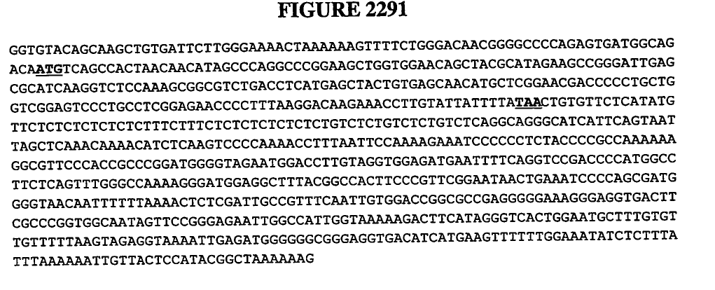

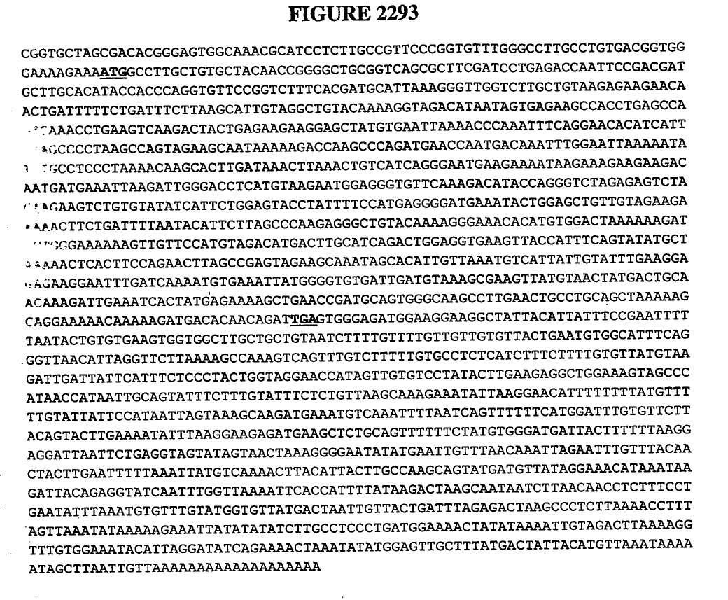

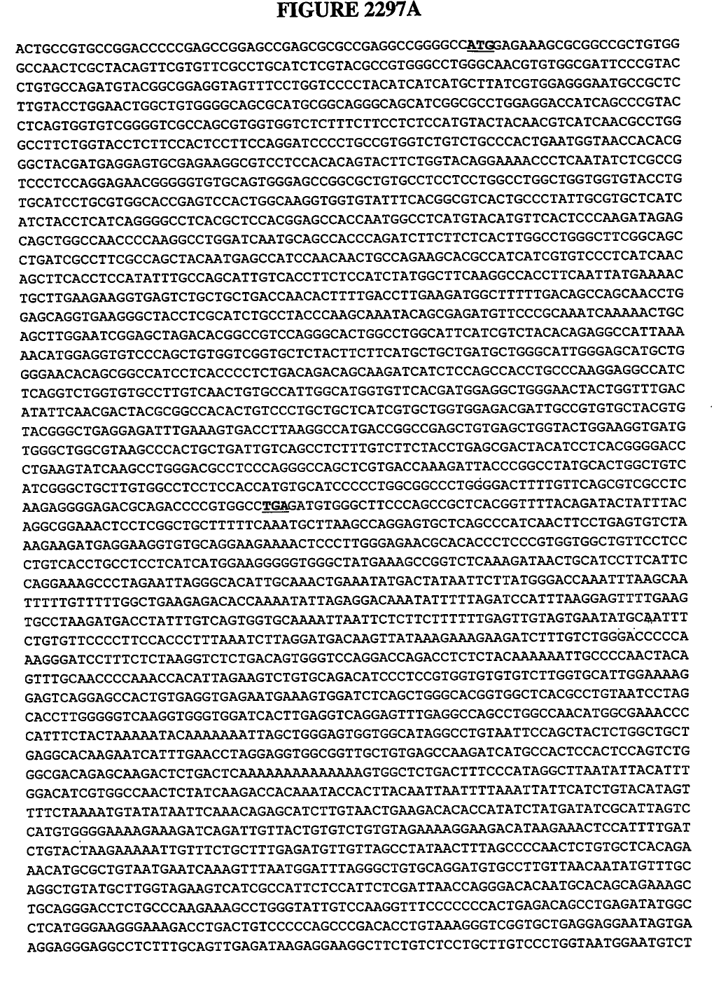

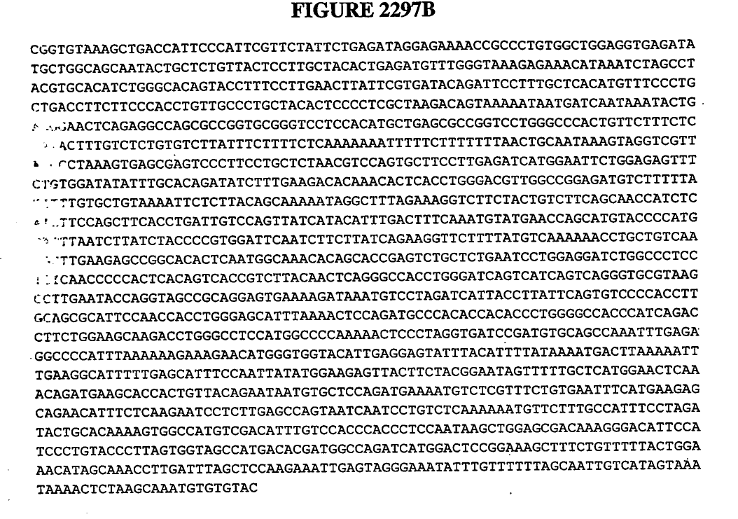

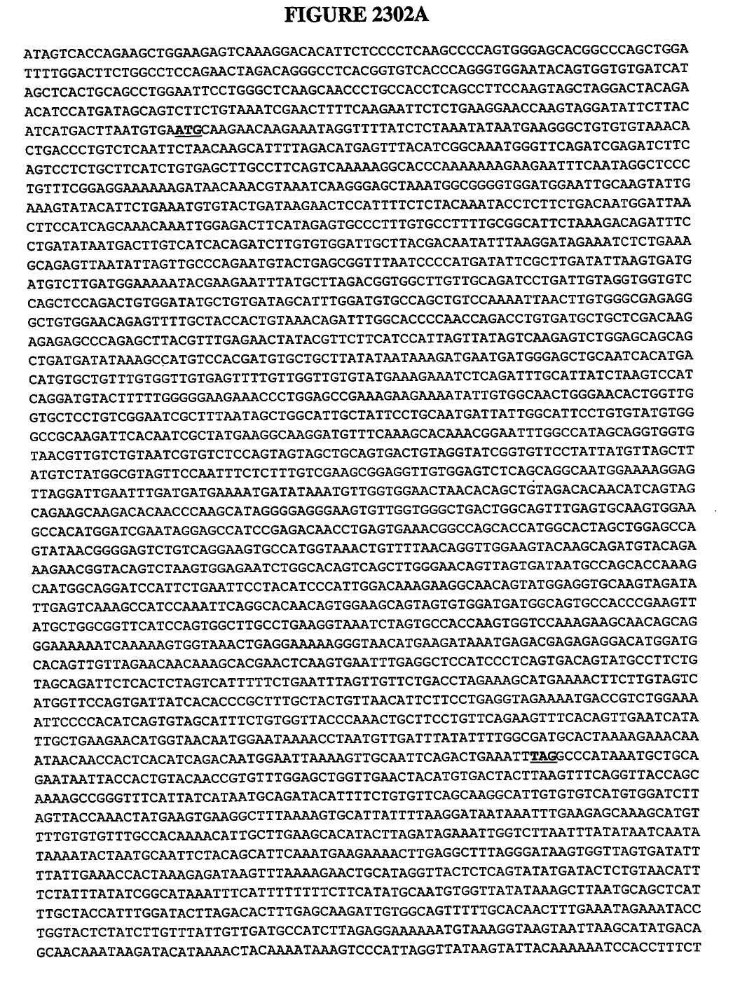

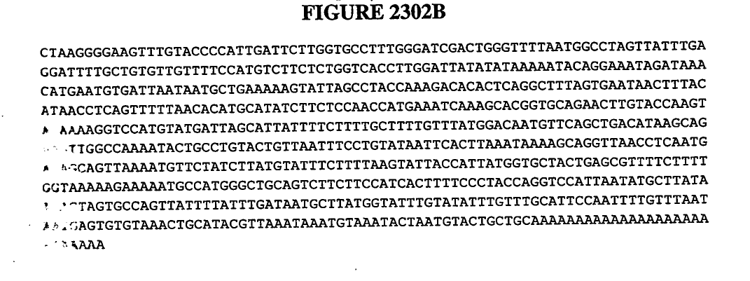

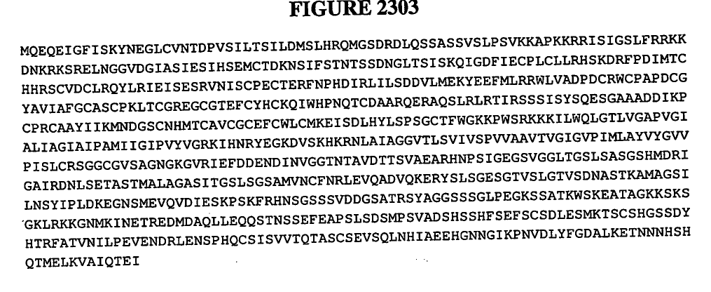

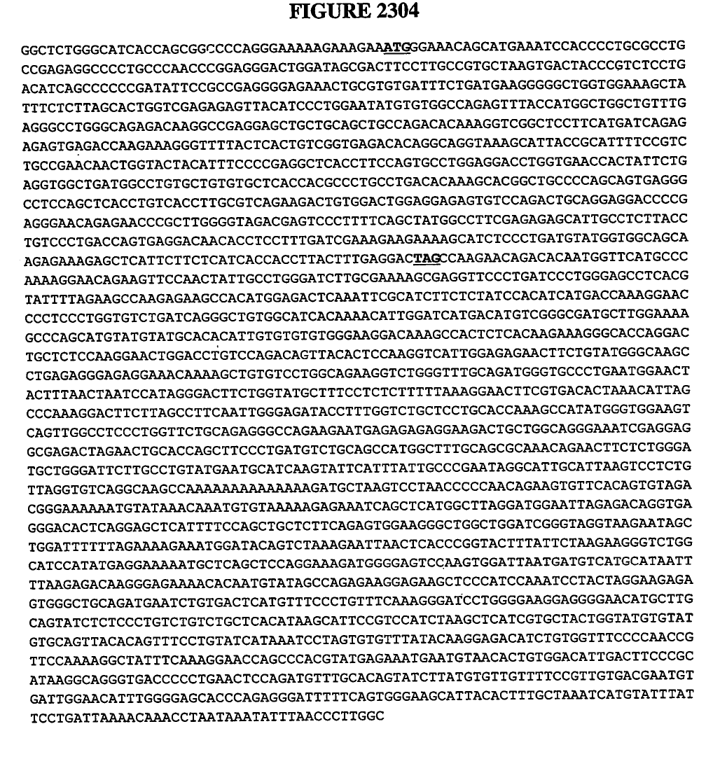

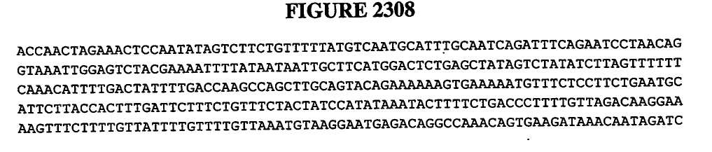

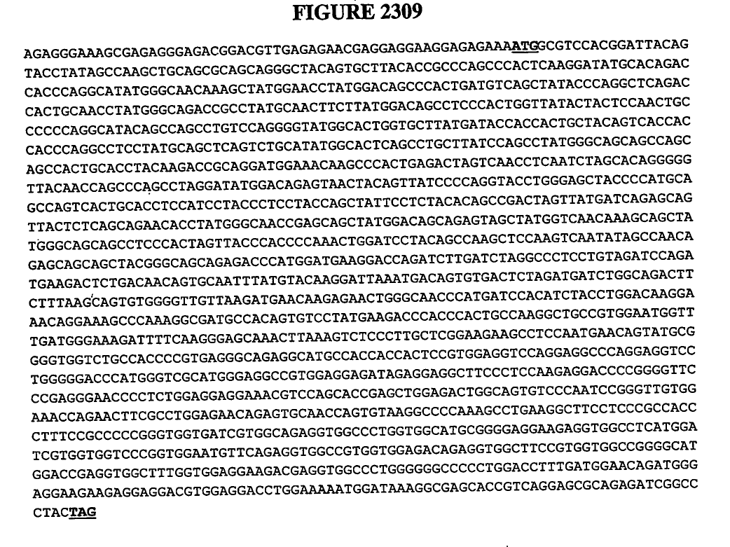

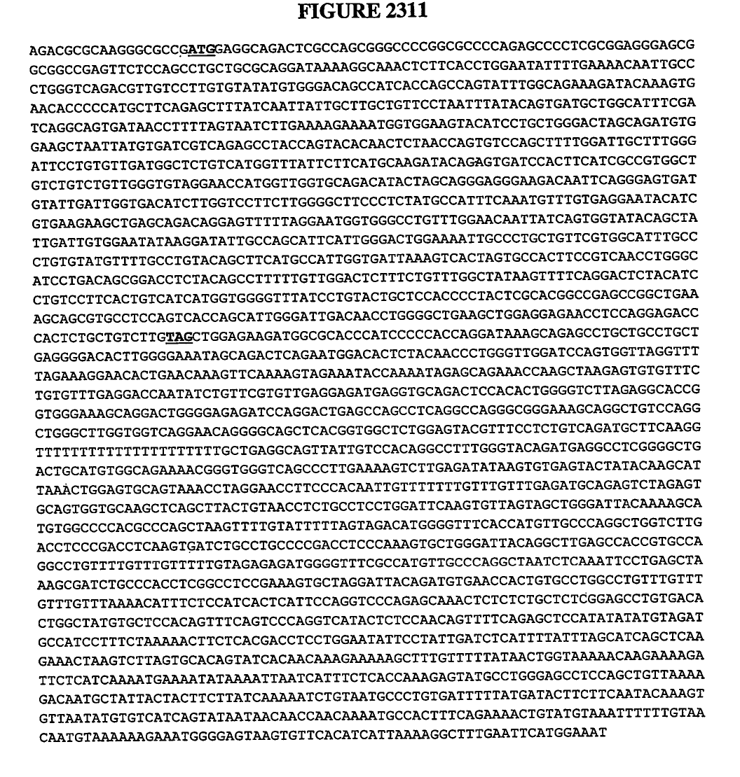

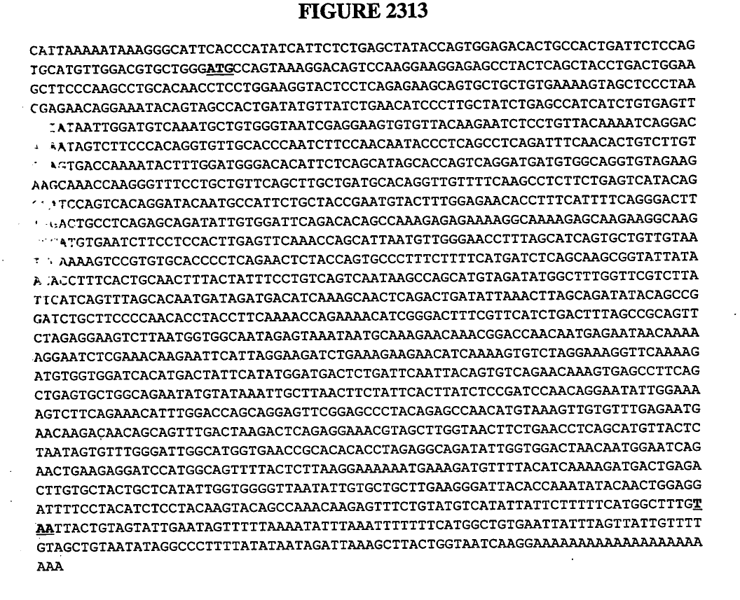

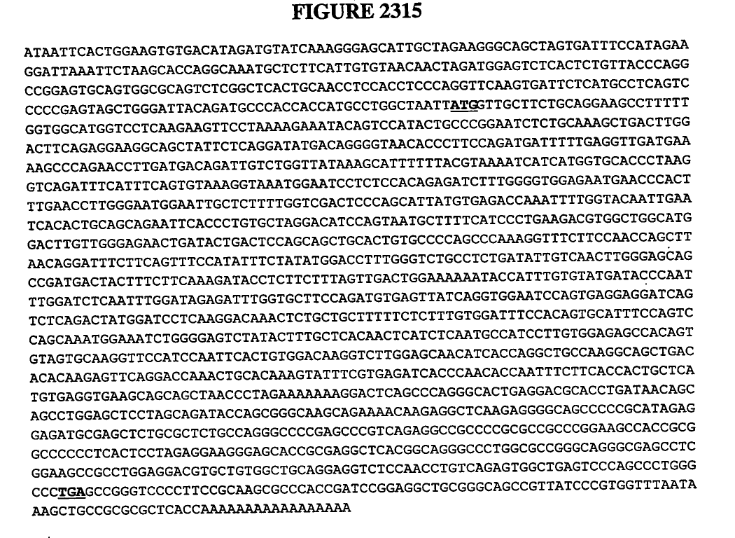

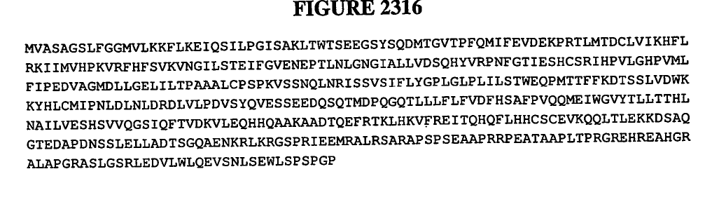

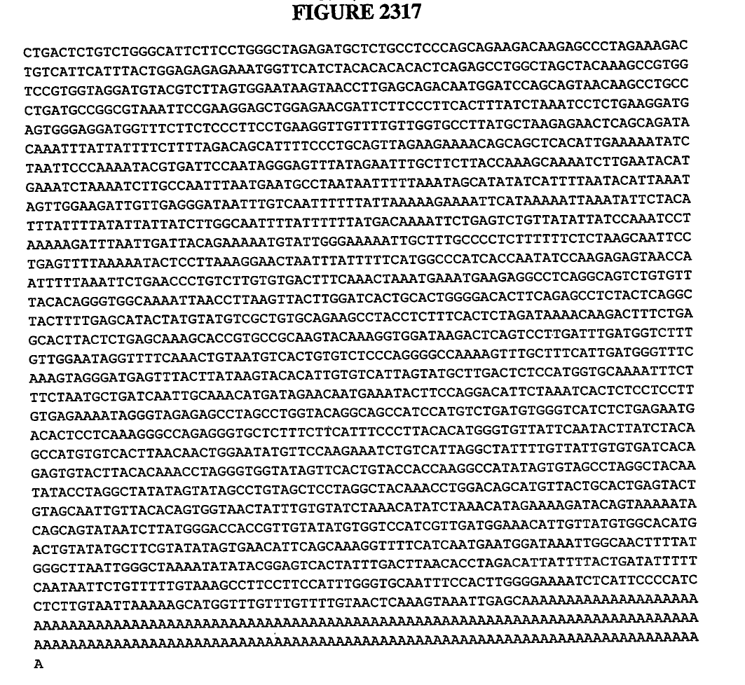

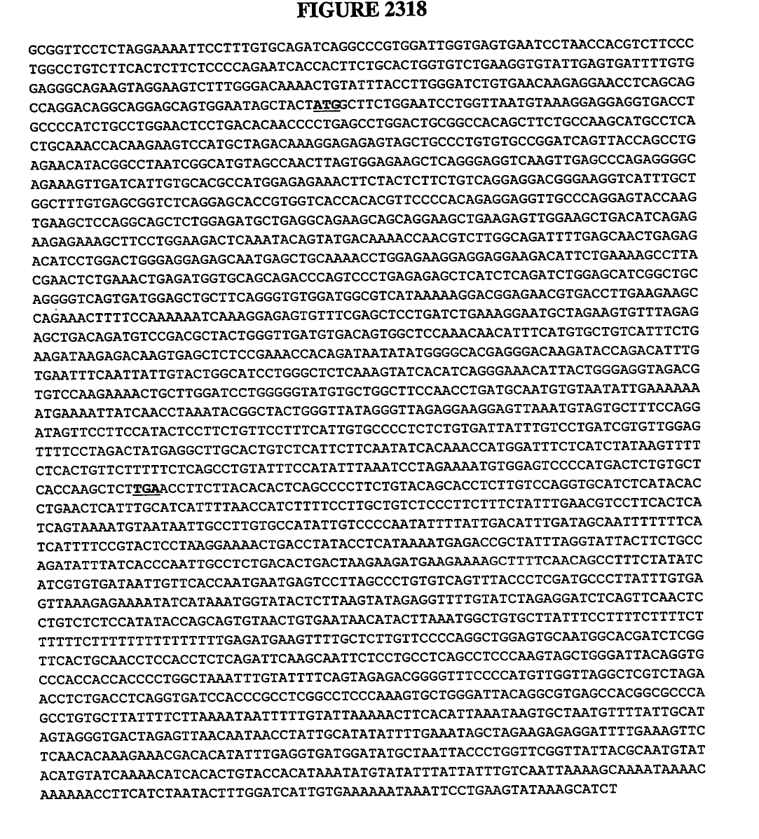

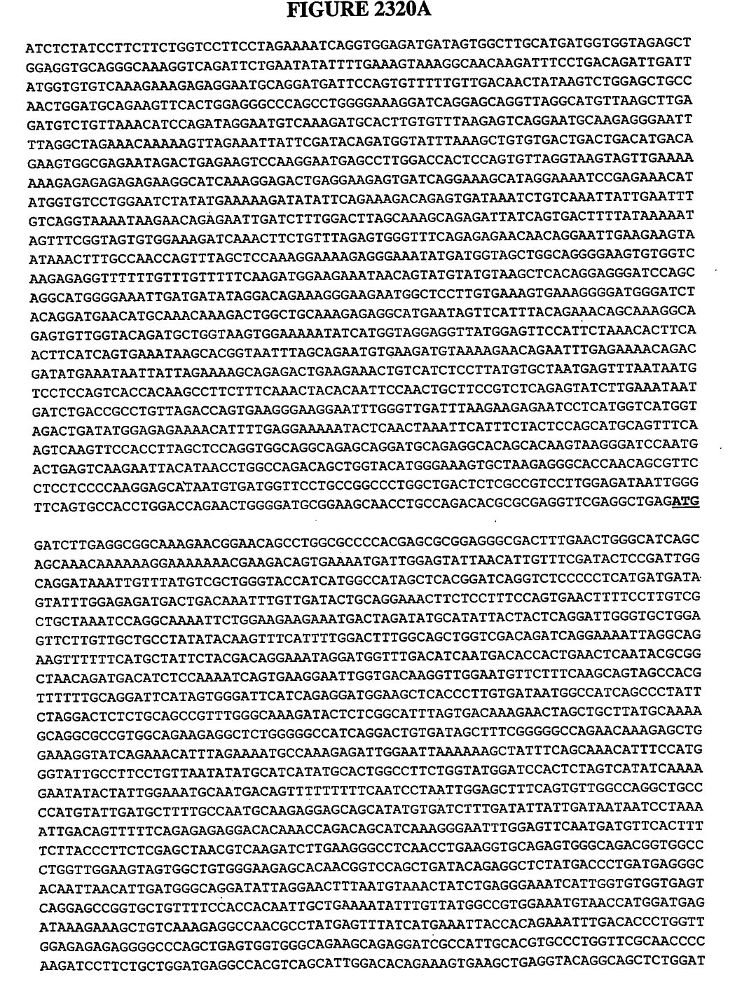

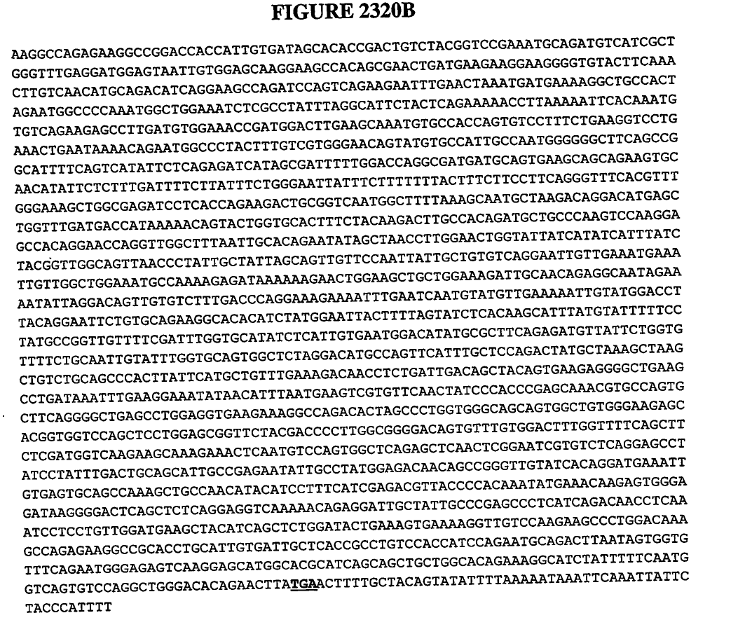

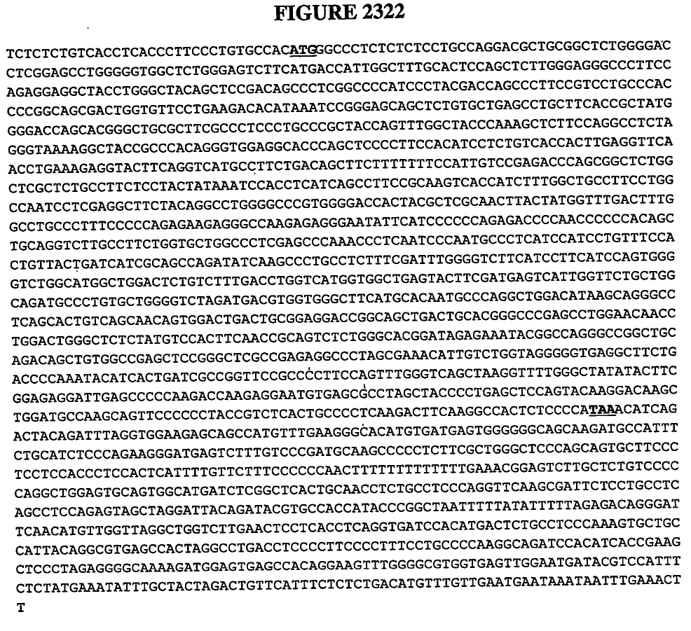

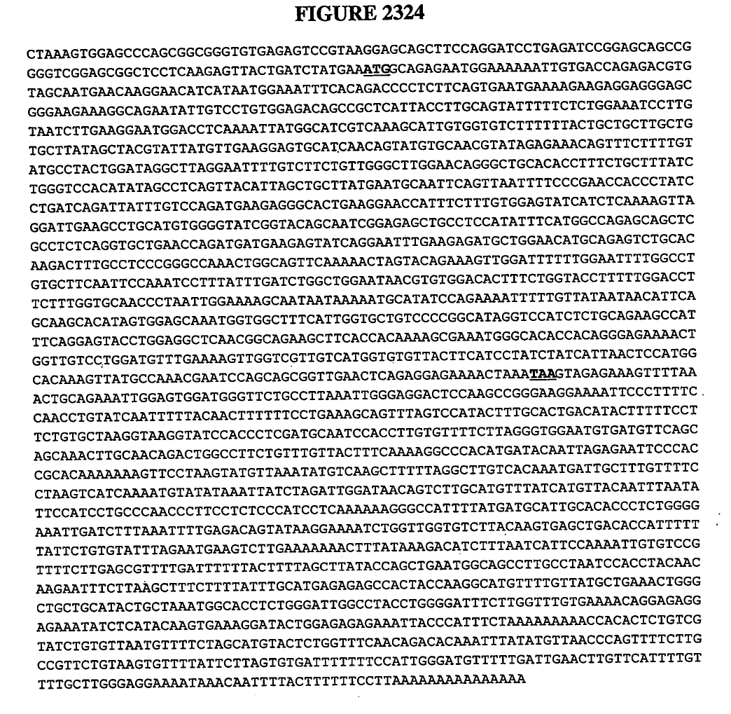

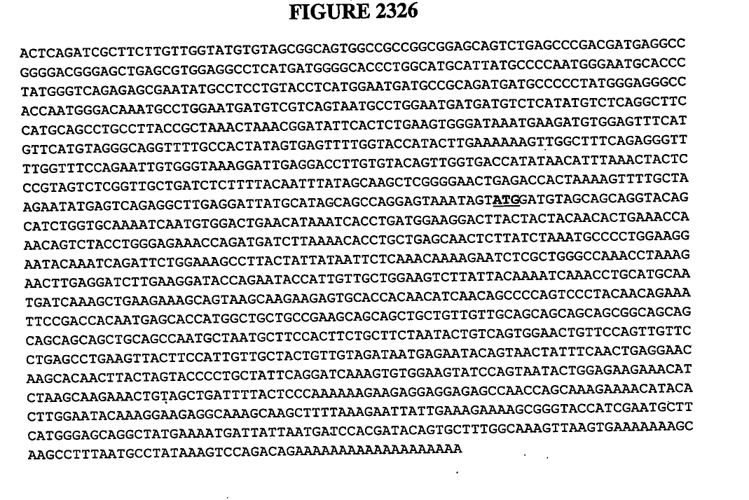

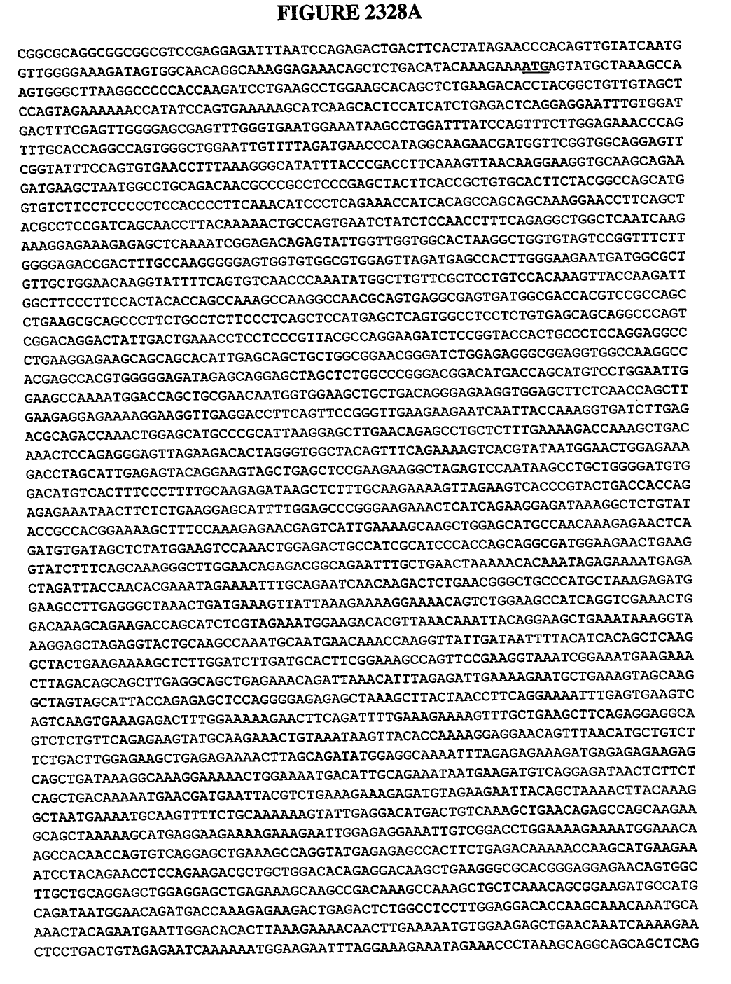

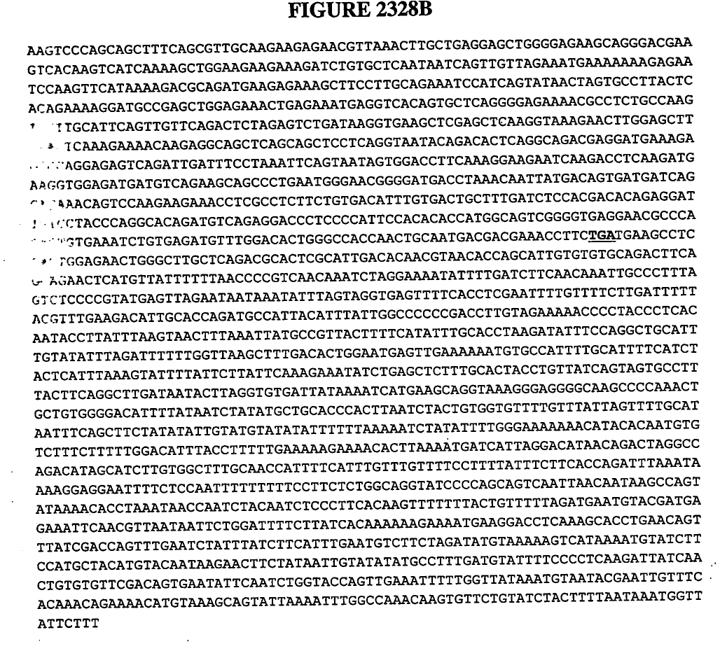

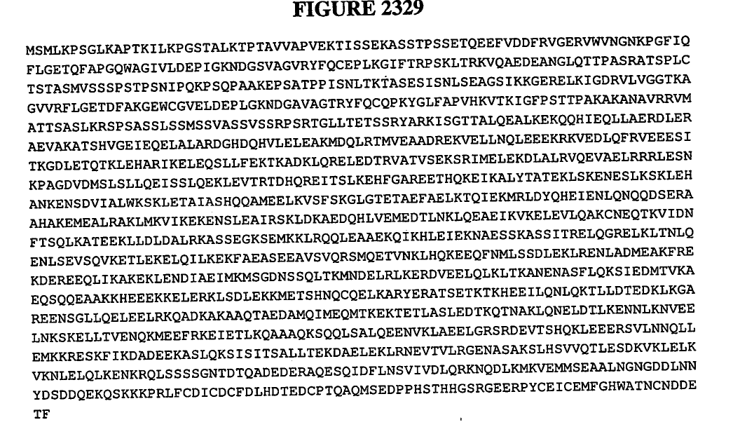

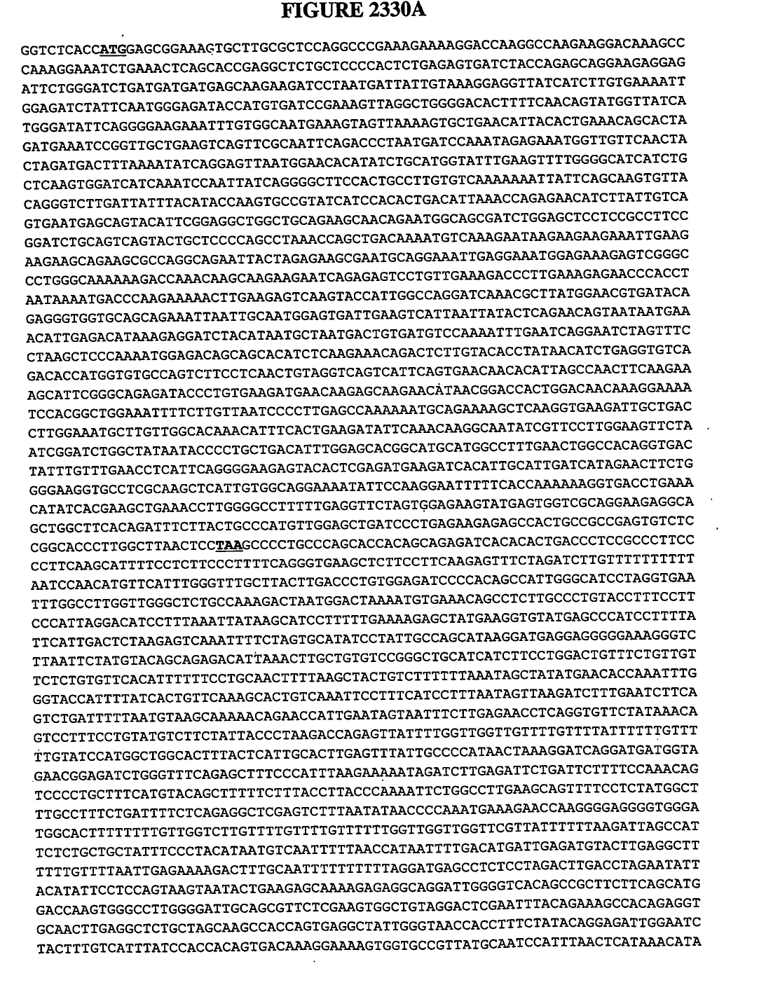

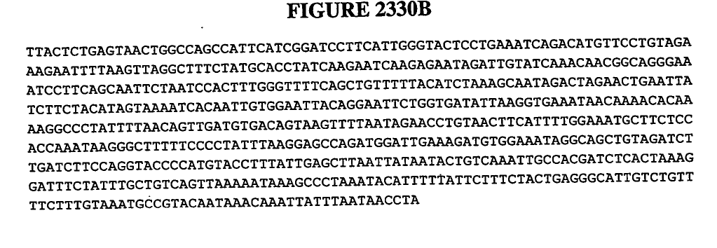

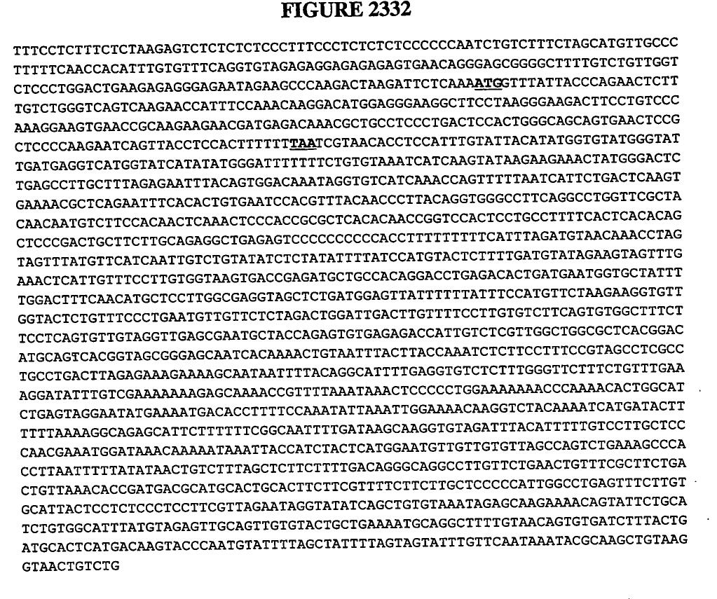

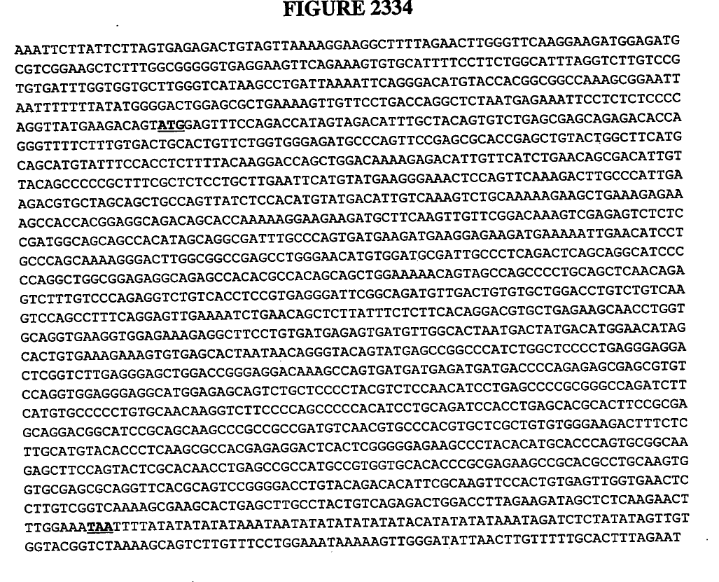

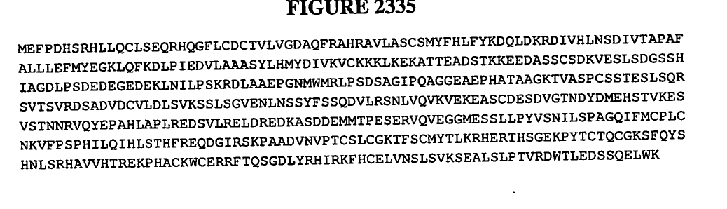

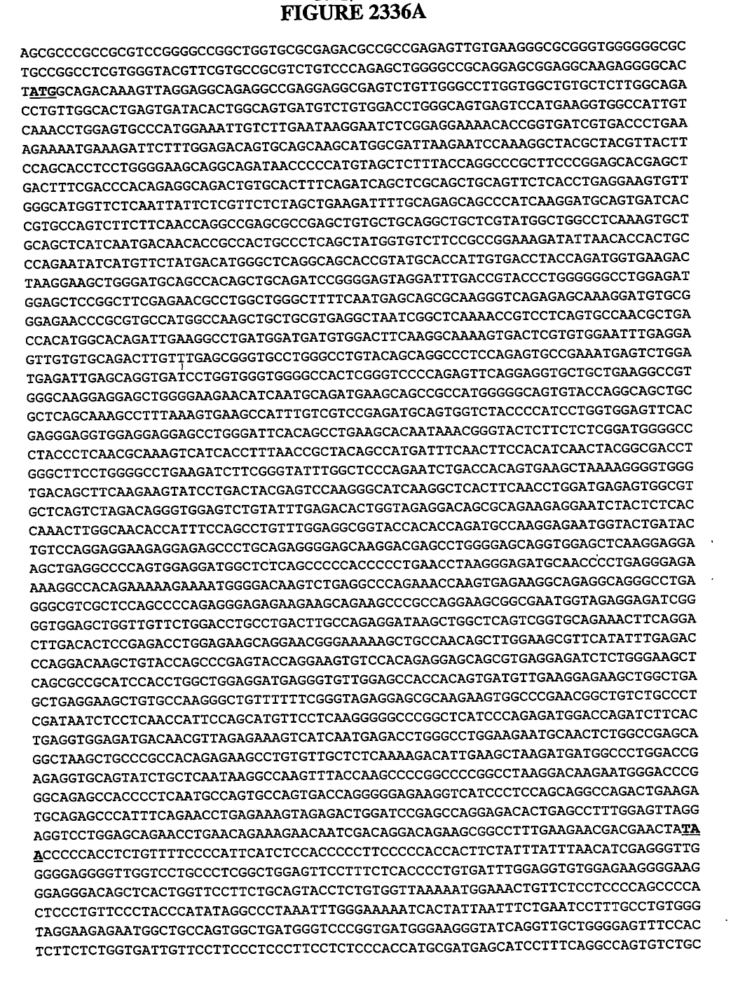

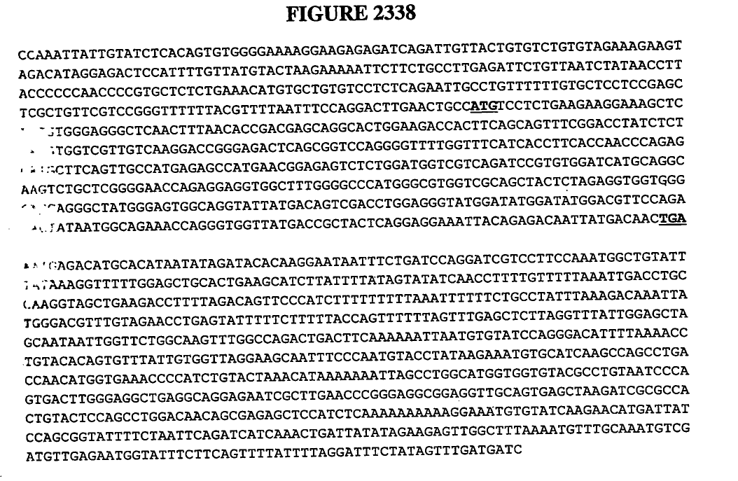

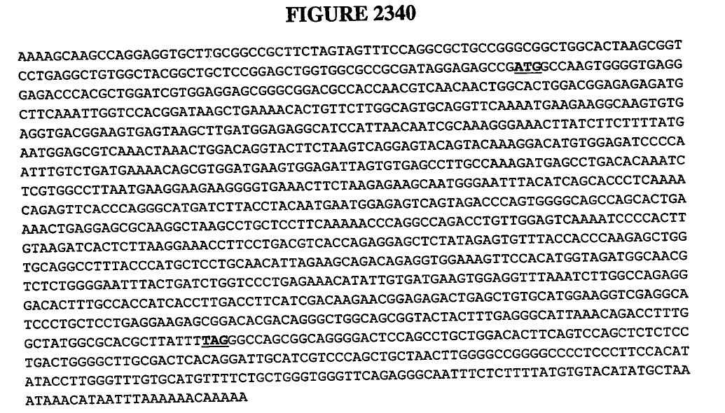

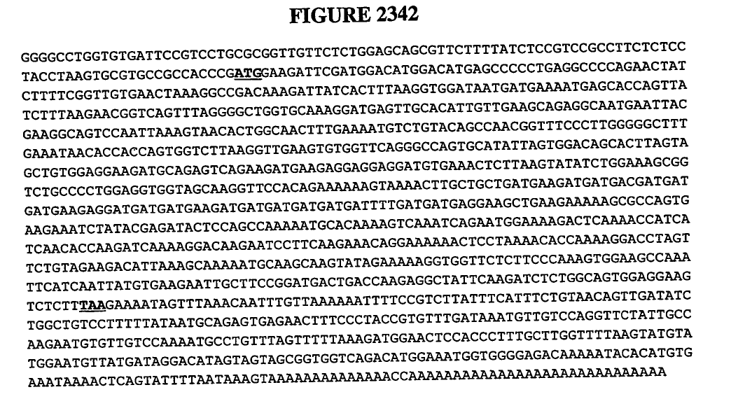

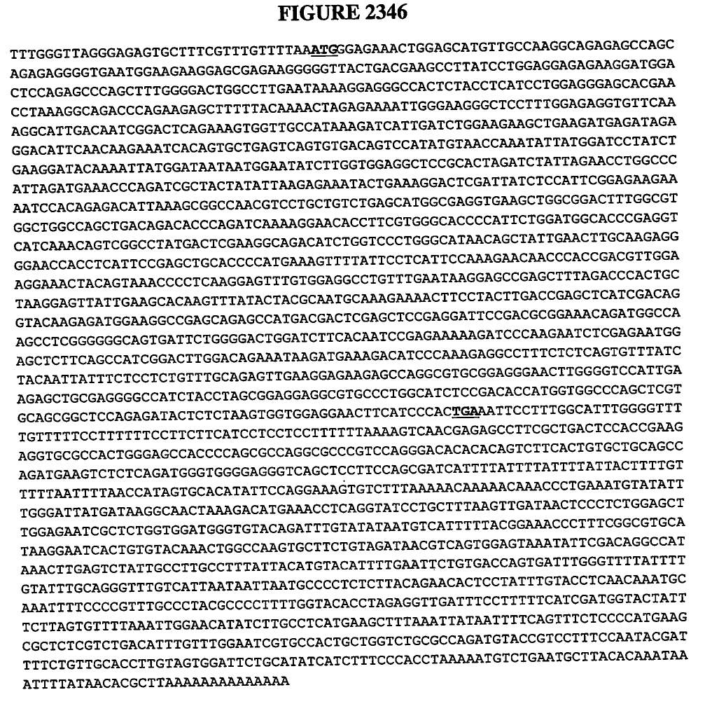

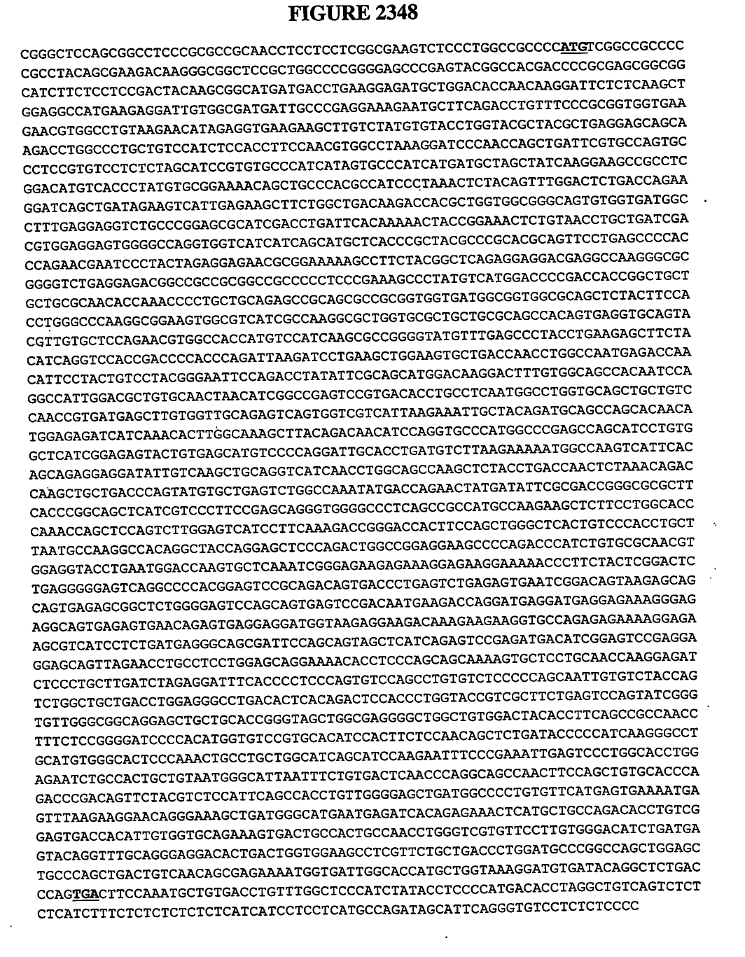

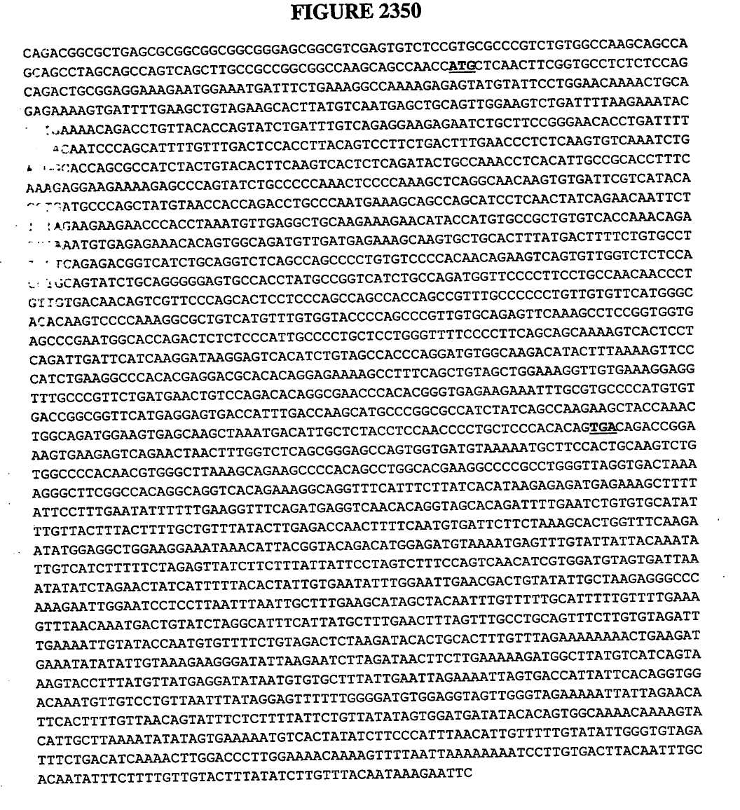

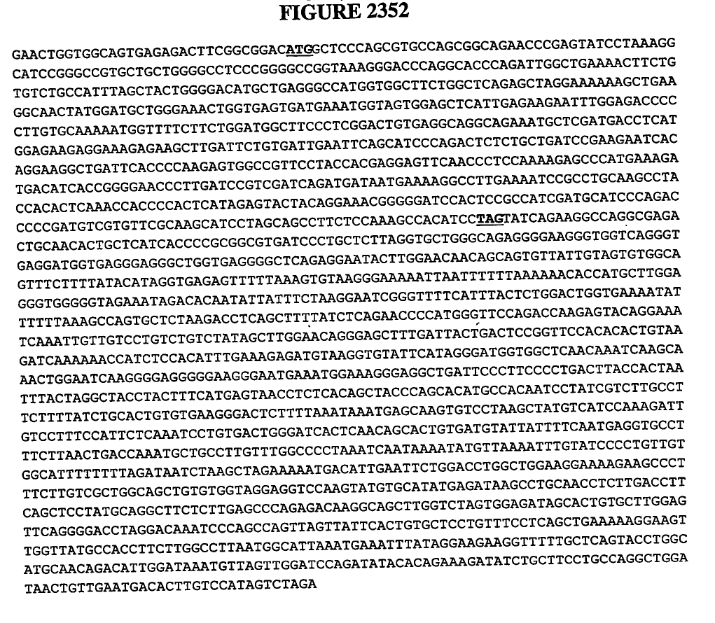

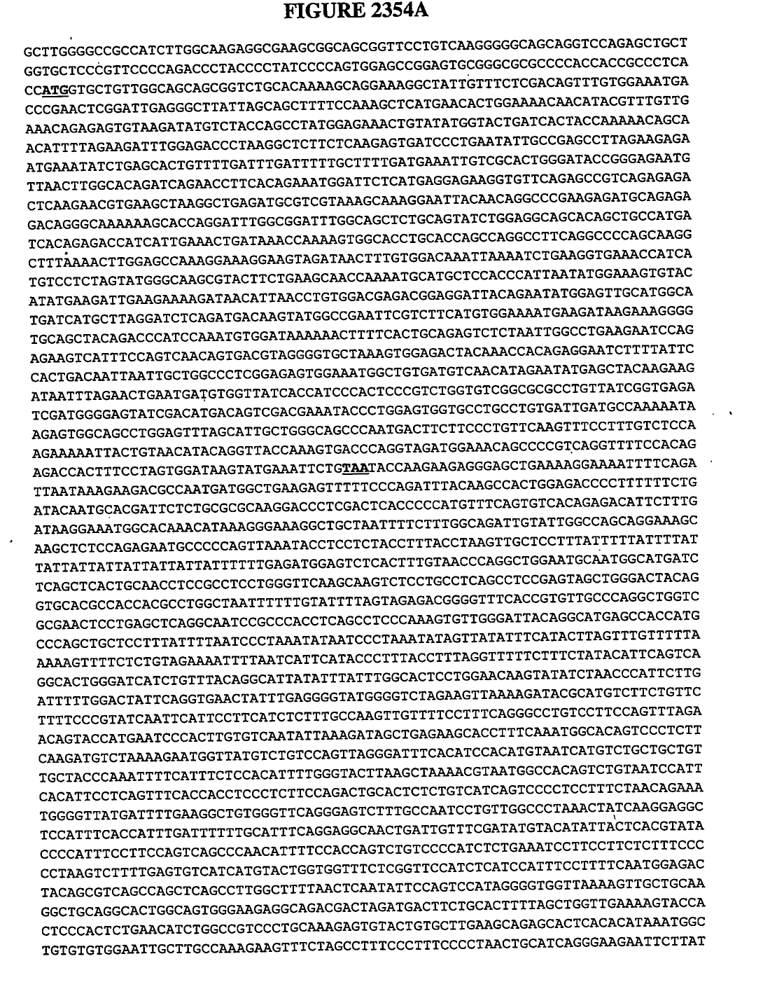

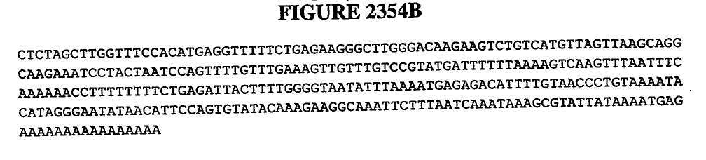

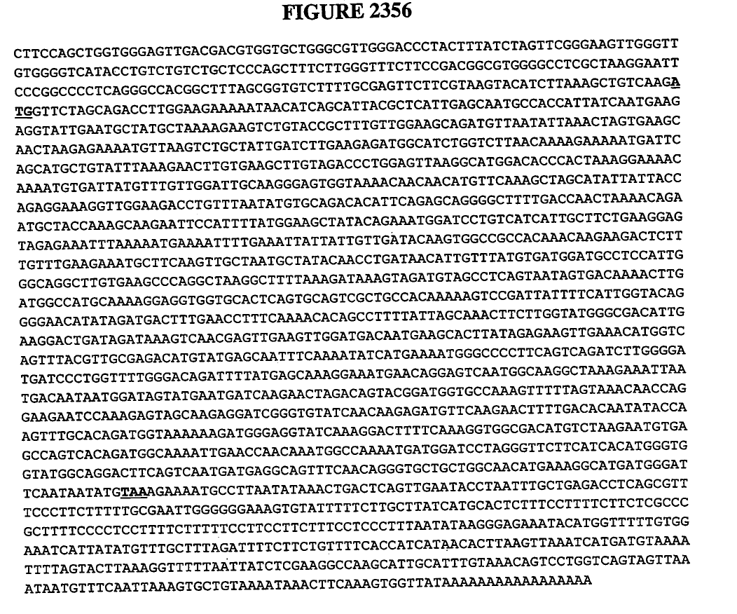

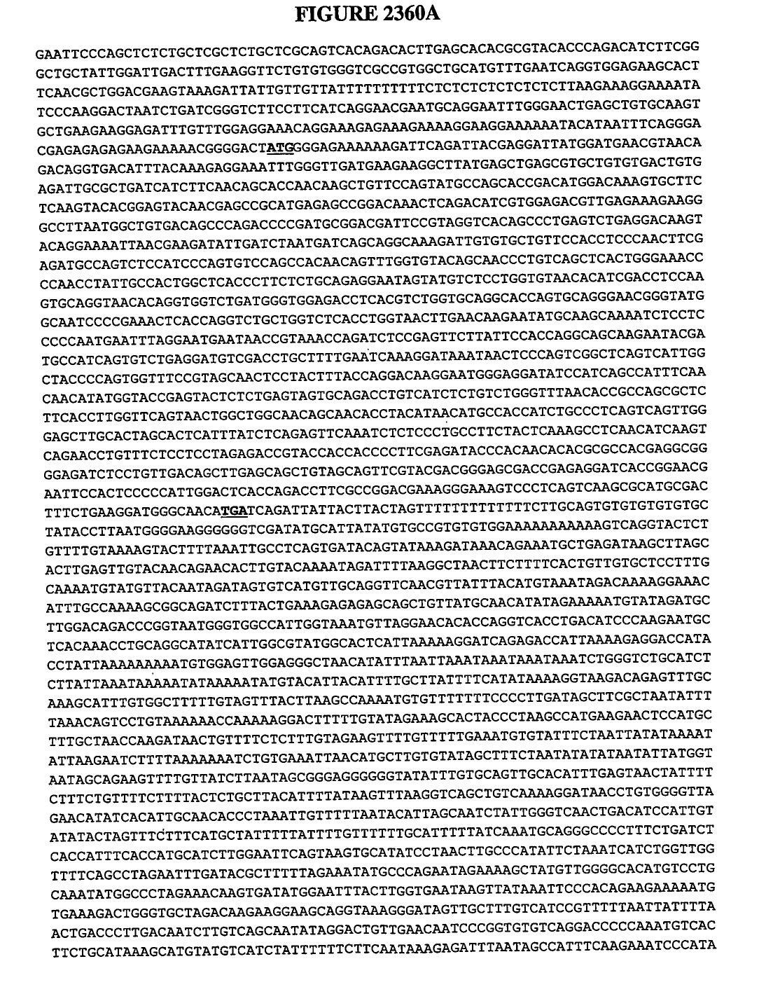

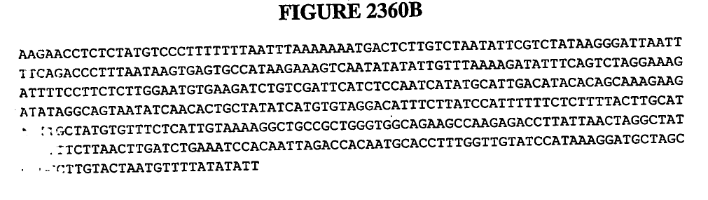

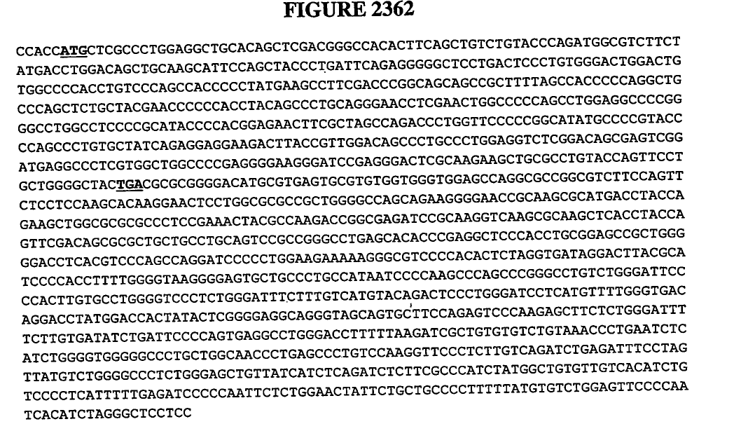

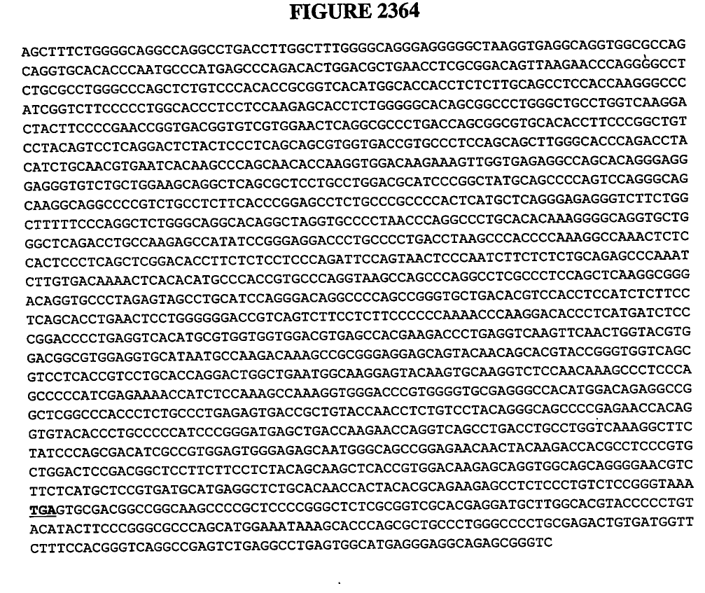

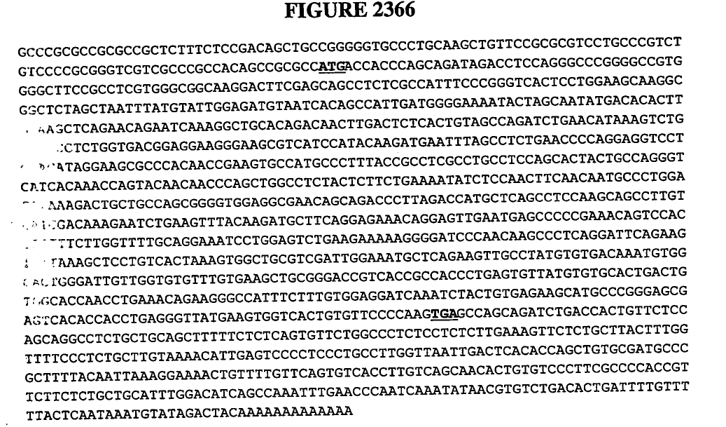

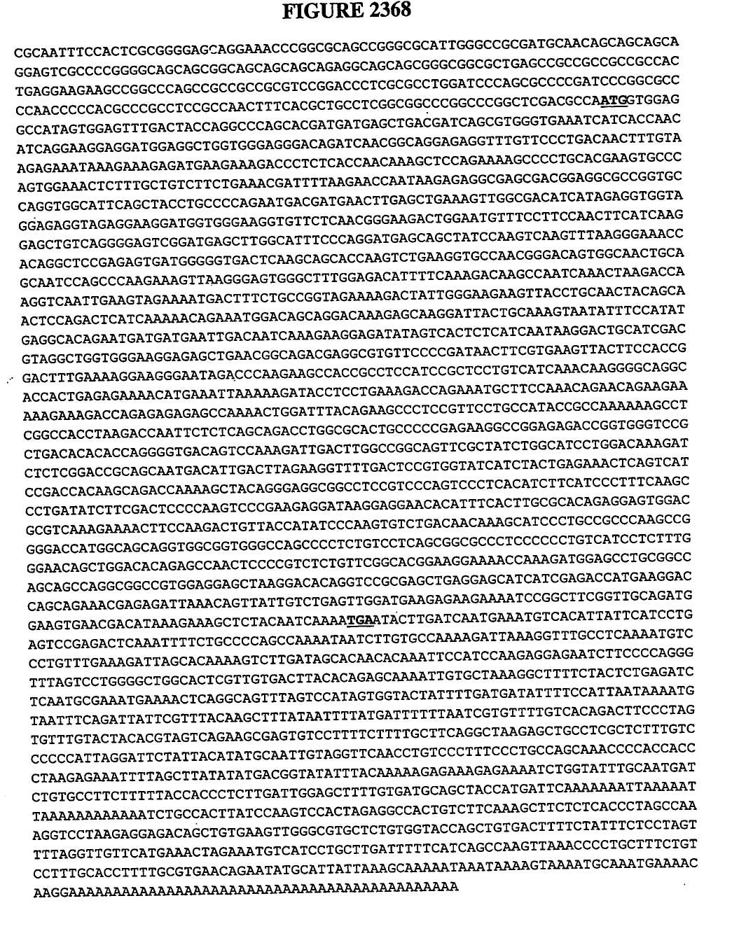

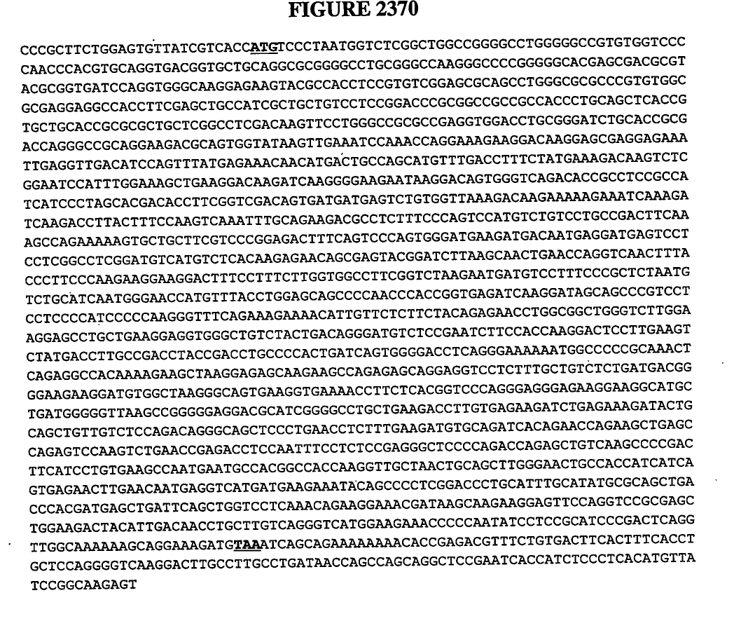

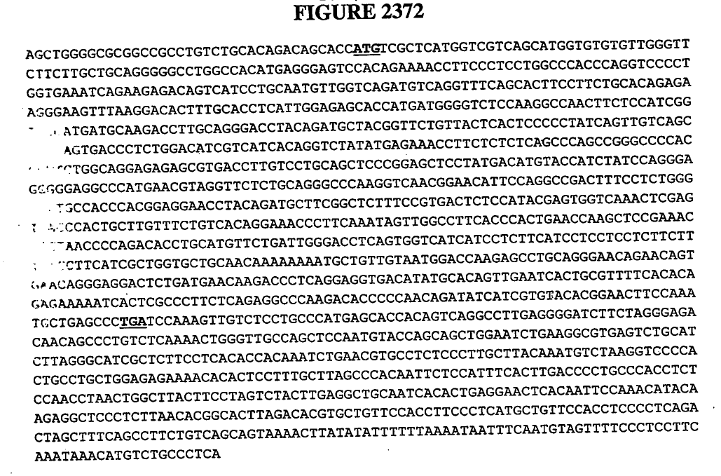

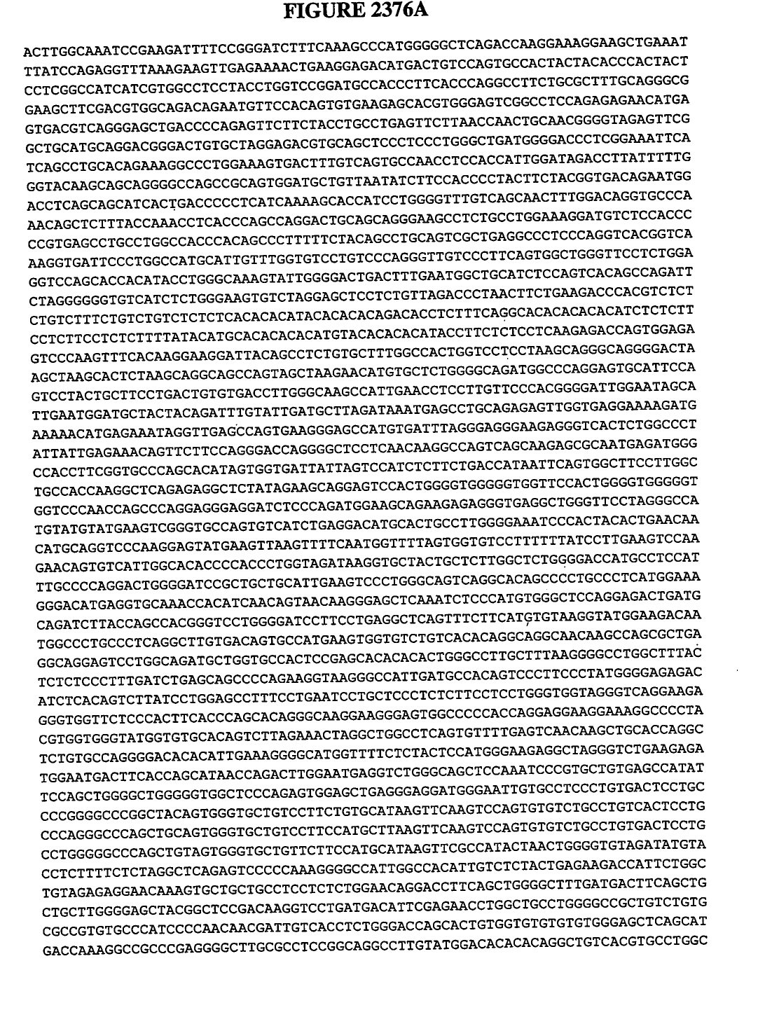

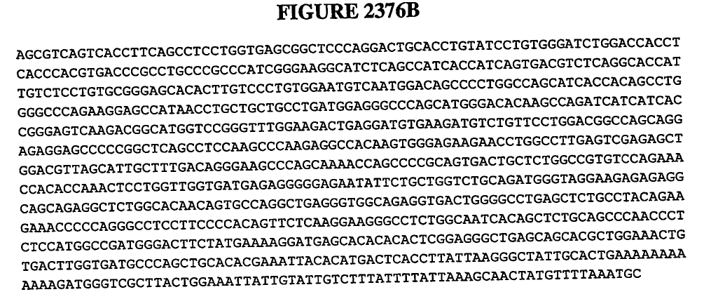

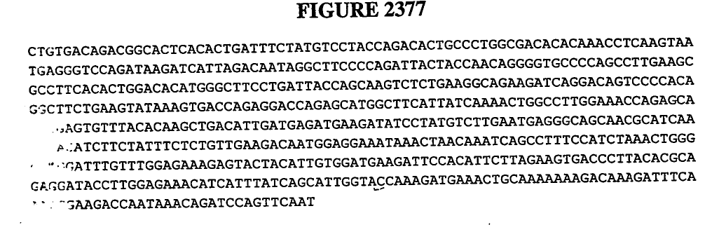

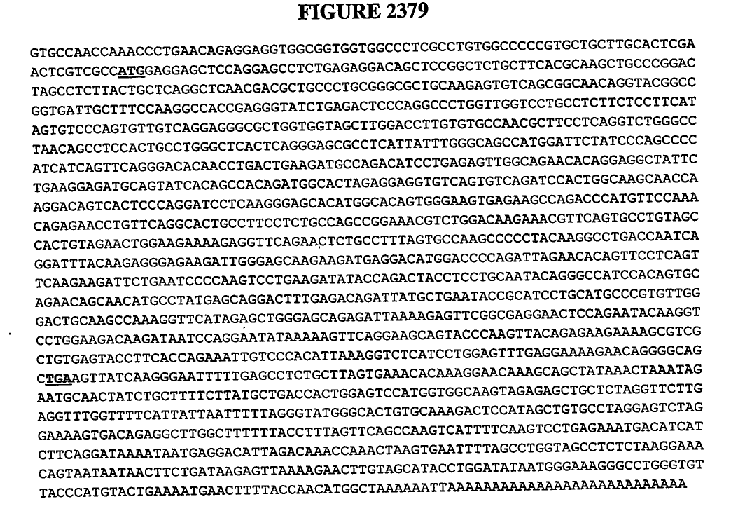

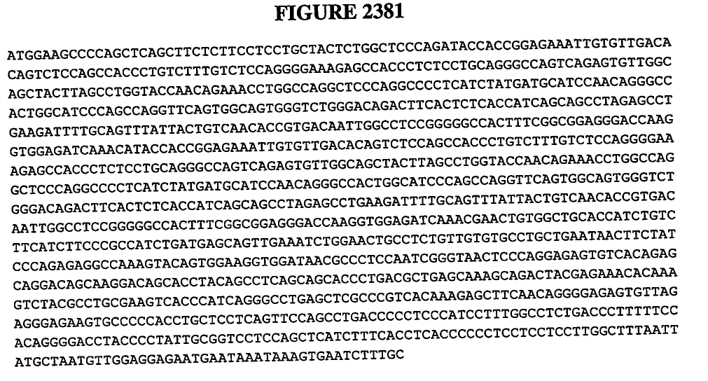

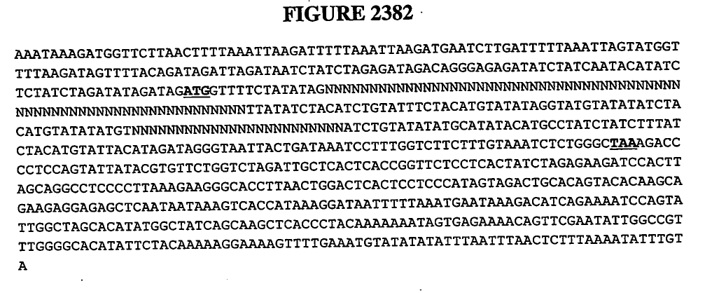

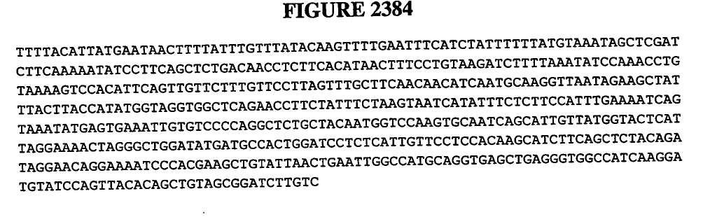

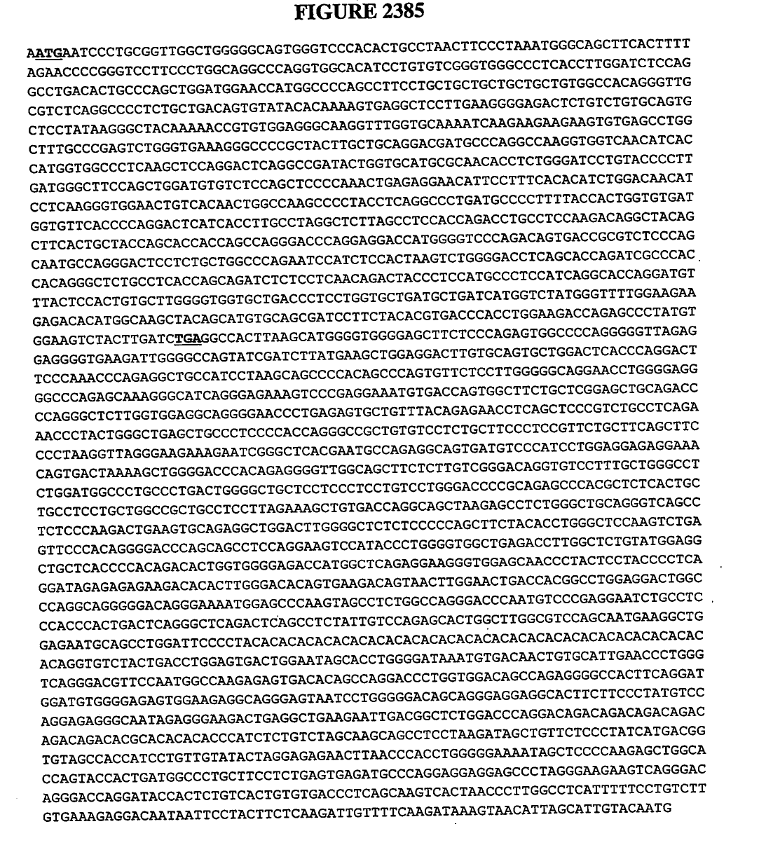

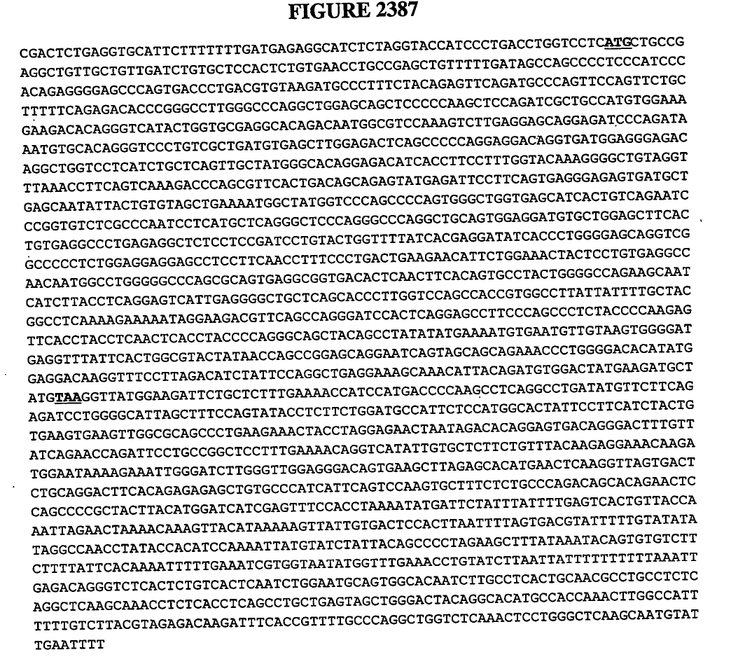

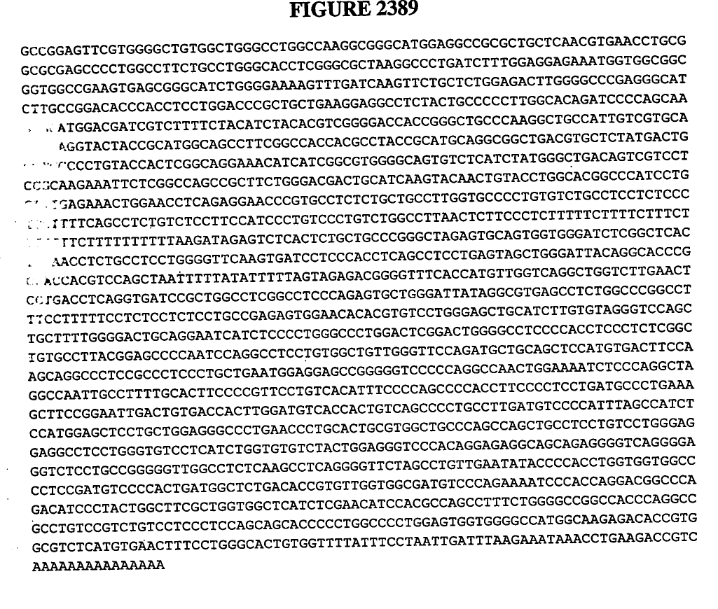

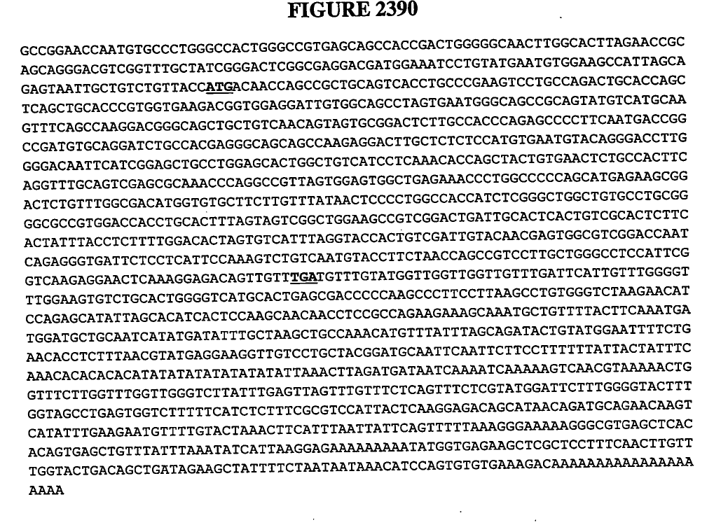

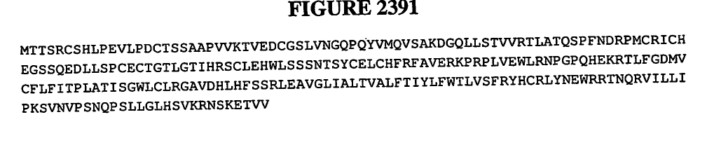

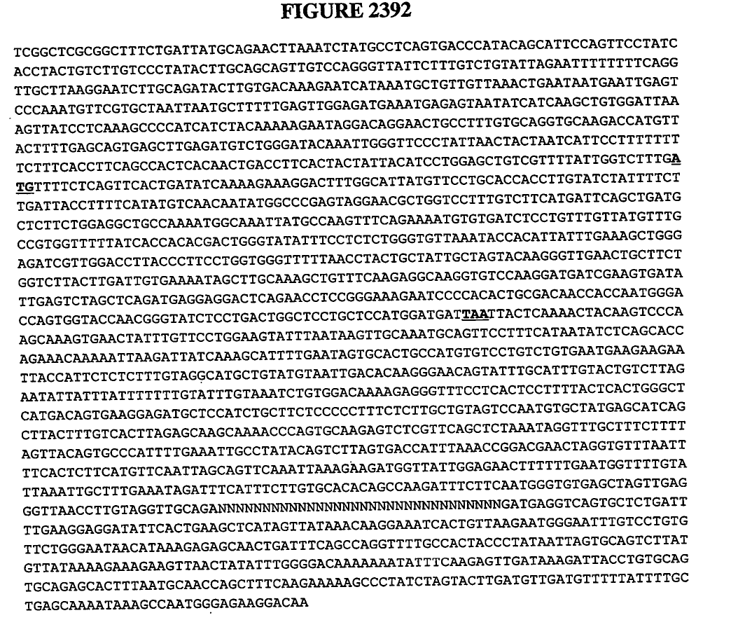

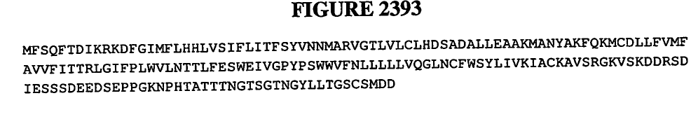

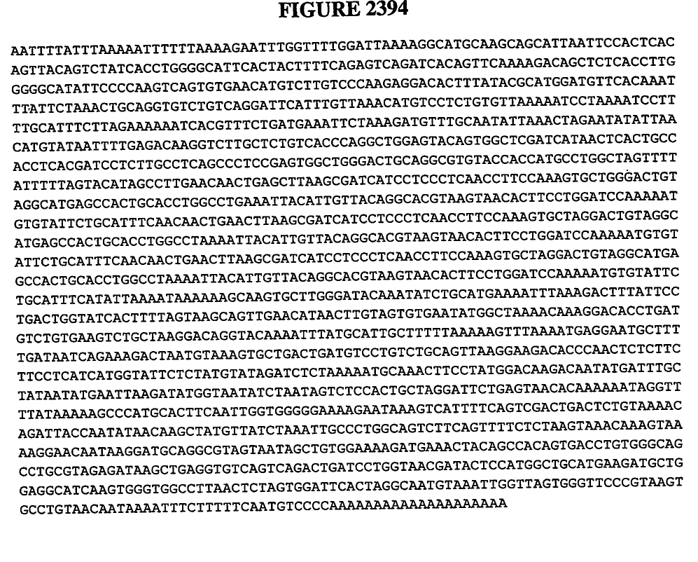

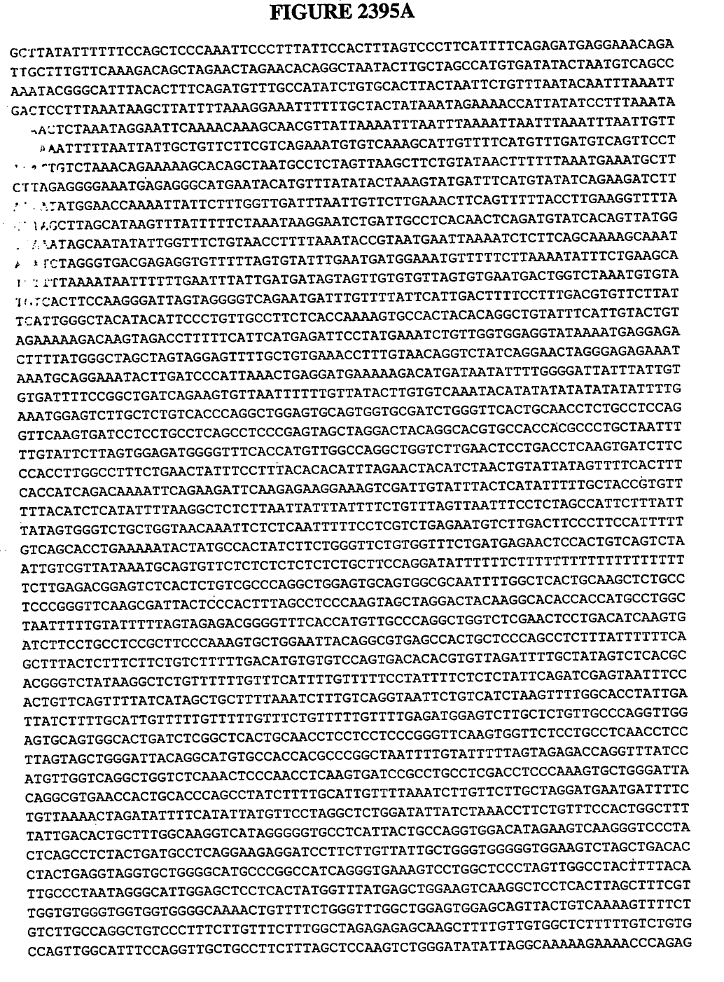

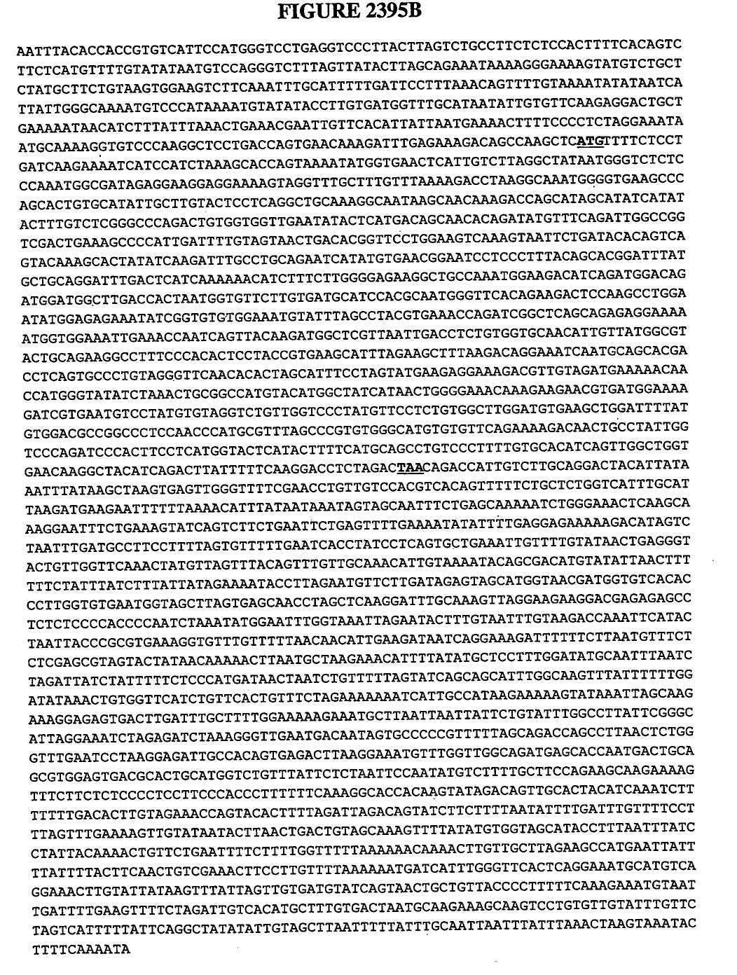

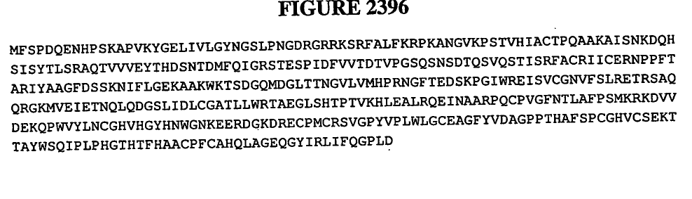

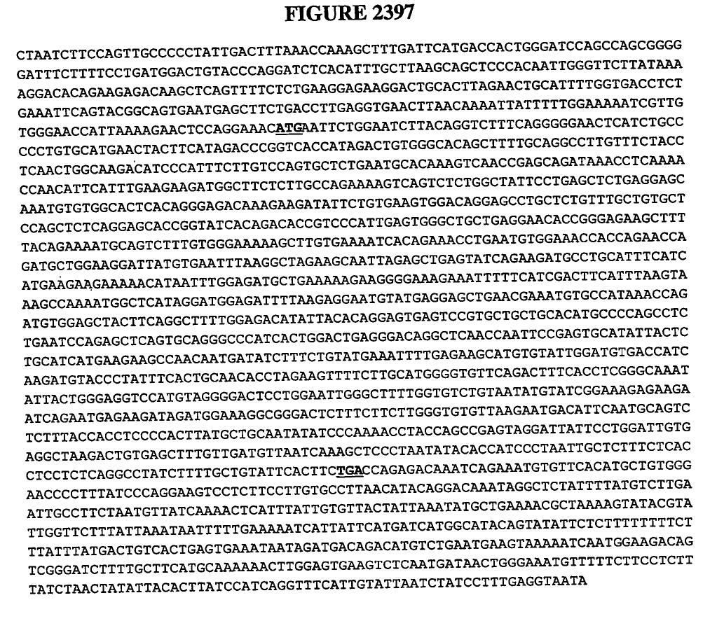

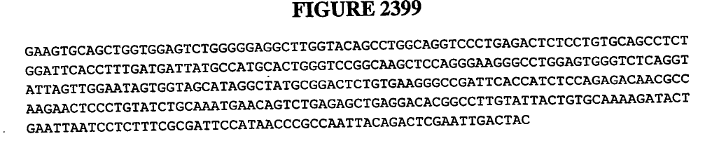

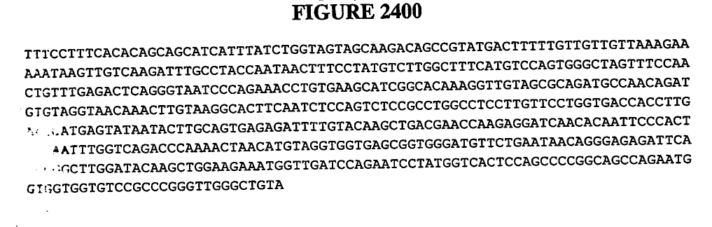

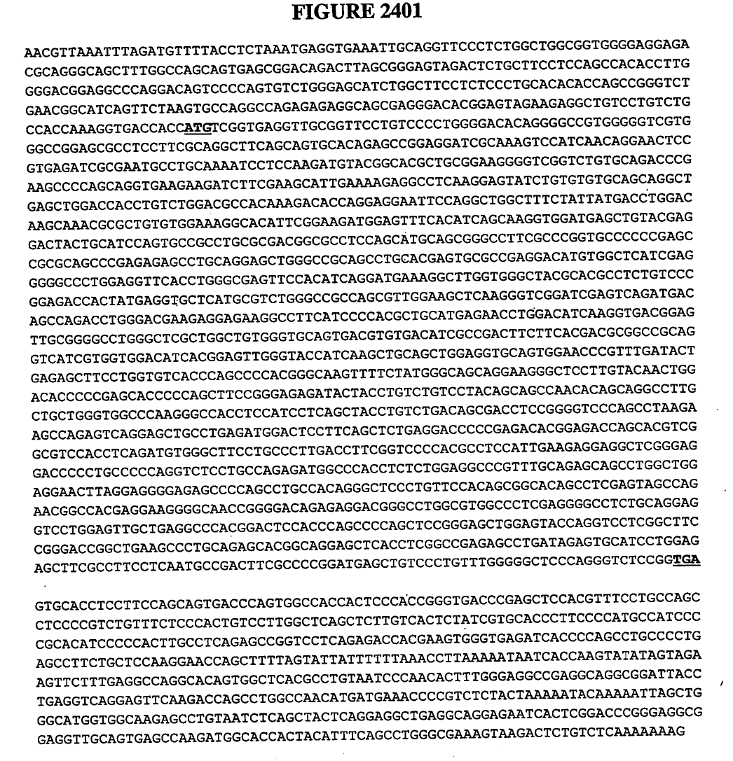

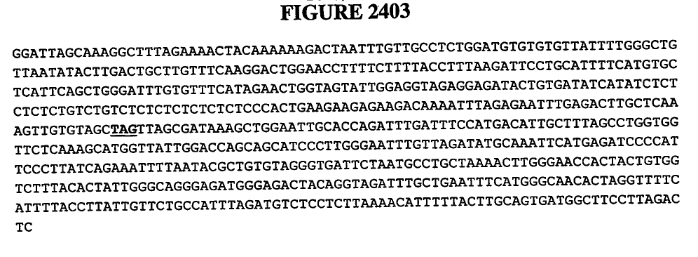

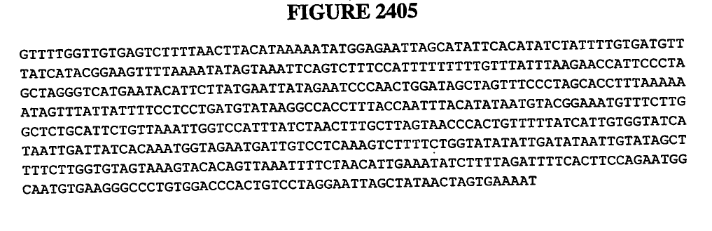

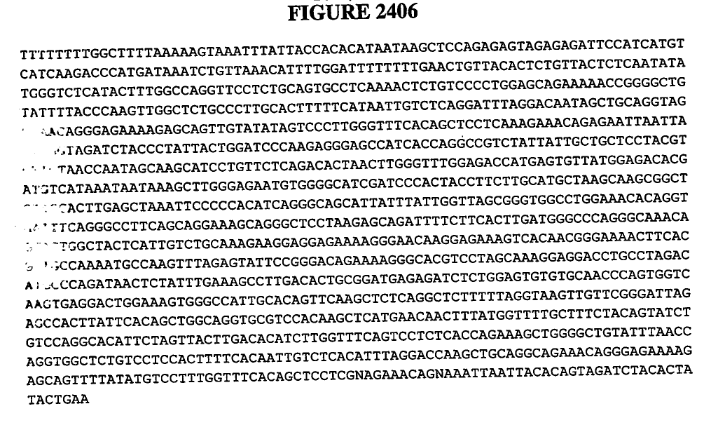

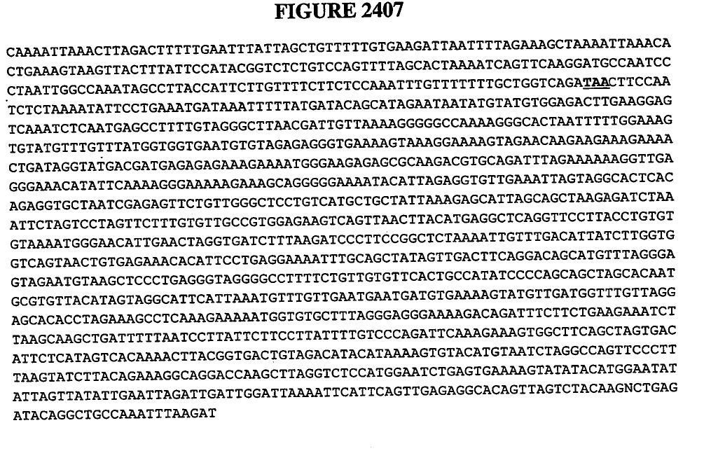

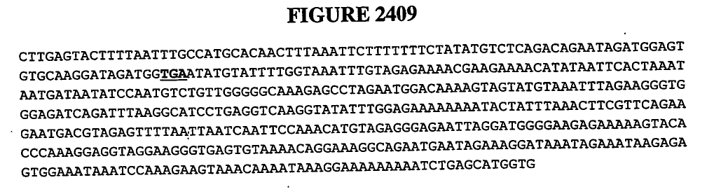

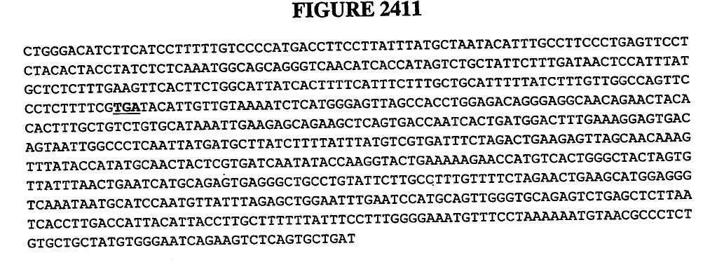

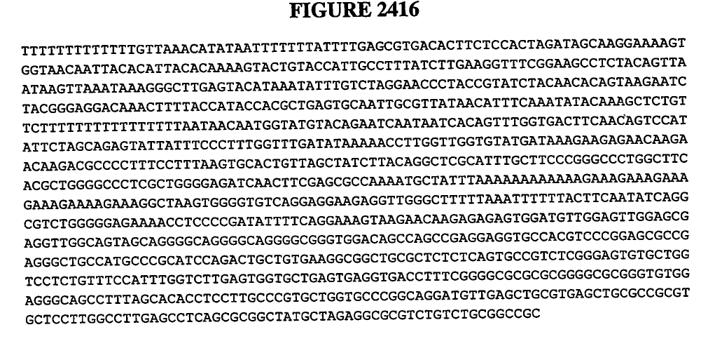

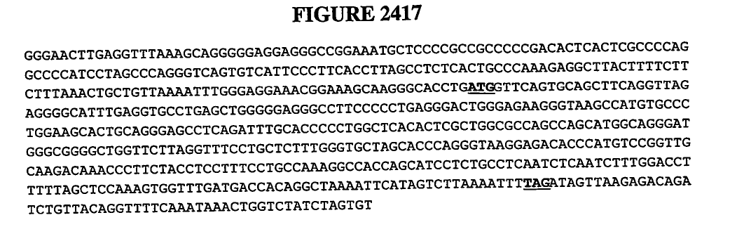

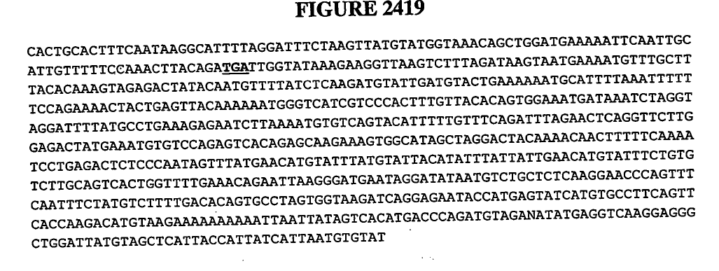

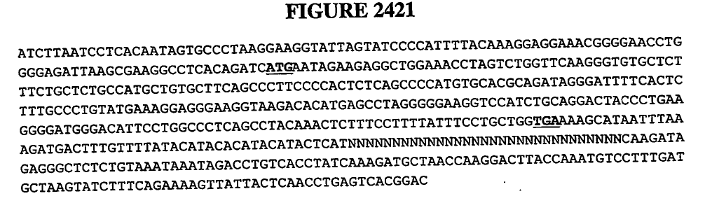

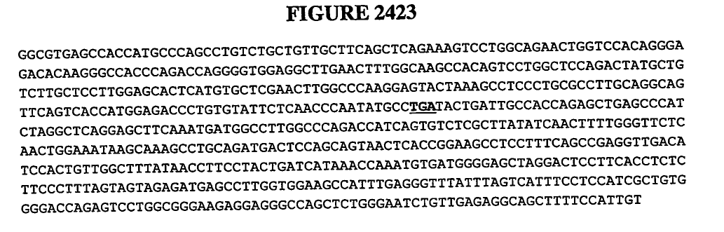

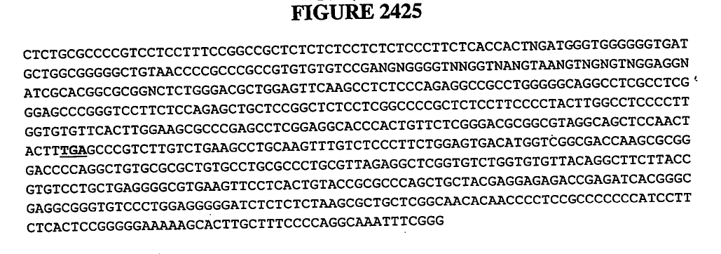

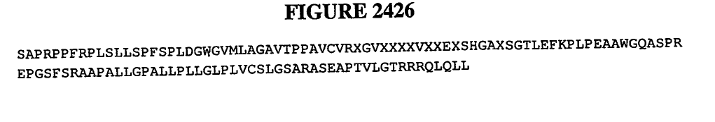

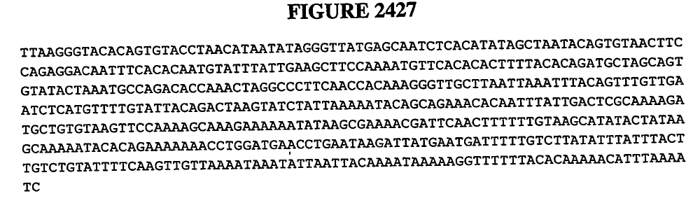

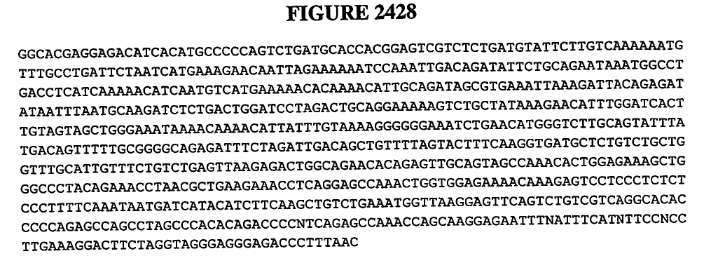

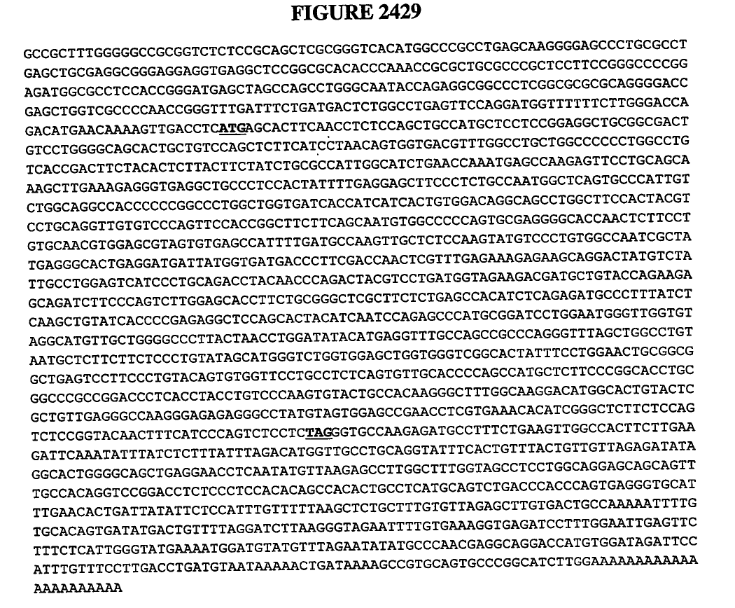

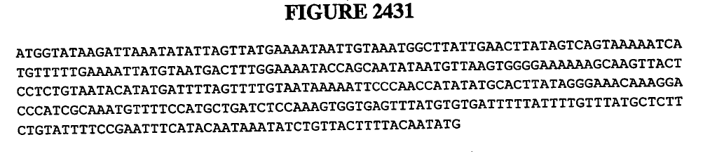

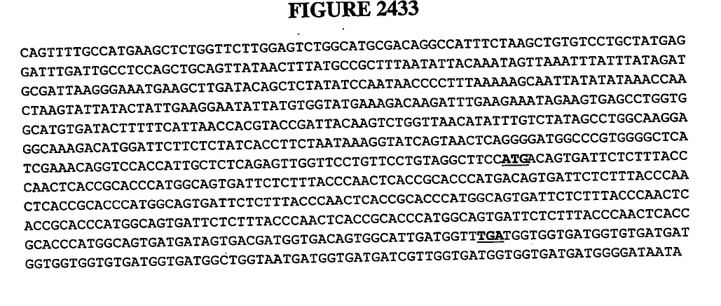

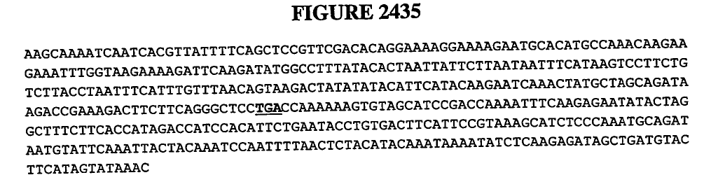

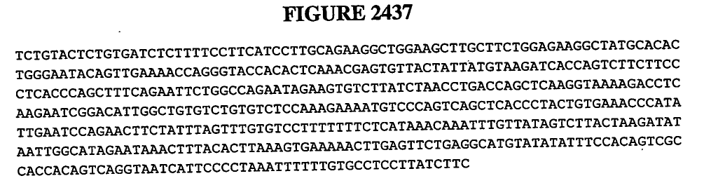

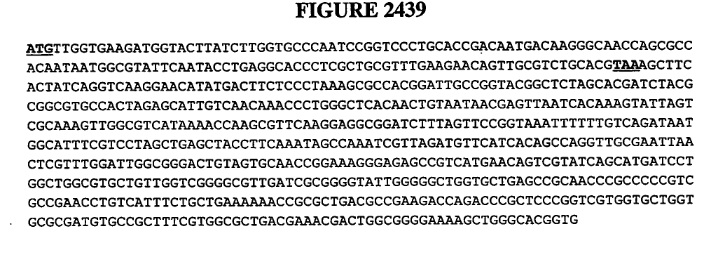

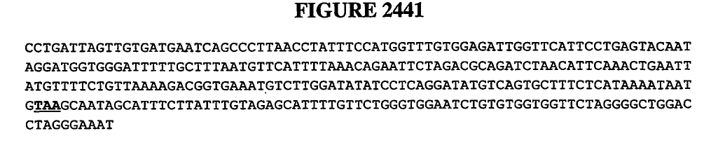

2. Isolated nucleic acid having at least 80% nucleic acid sequence identity to a nucleotide sequence selected from the group consisting of the nucleotide sequence shown in FIG. 1 (SEQ ID NO:1), FIG. 3 (SEQ ID NO:3), FIG. 5 (SEQ ID NO:5), FIG. 7 (SEQ ID NO:7), FIG. 9 (SEQ ID NO:9), FIG. 11 (SEQ ID NO:11), FIG. 13 (SEQ ID NO:13), FIG. 15 (SEQ ID NO:15), FIG. 17 (SEQ ID NO:17), FIG. 19 (SEQ ID NO:19), FIG. 21 (SEQ ID NO:21), FIG. 23 (SEQ ID NO:23), FIG. 25 (SEQ ID NO:25), FIG. 27 (SEQ ID NO:27), FIG. 29 (SEQ ID NO:29), FIG. 31 (SEQ ID NO:31), FIG. 33 (SEQ ID NO:33), FIG. 35 (SEQ ID NO:35), FIG. 37 (SEQ ID NO:37), FIG. 39 (SEQ ID NO:39), FIG. 41 (SEQ ID NO:41), FIG. 43 (SEQ ID NO:43), FIG. 45 (SEQ ID NO:45), FIG. 47 (SEQ ID NO:47), FIG. 49 (SEQ ID NO:49), FIG. 51 (SEQ ID NO:51), FIG. 53 (SEQ ID NO:53), FIG. 55 (SEQ ID NO:55), FIG. 57 (SEQ ID NO:57), FIG. 59 (SEQ ID NO:59), FIG. 61 (SEQ ID NO:61), FIG. 63 (SEQ ID NO:63), FIG. 65 (SEQ ID NO:65), FIG. 67 (SEQ ID NO:67), FIG. 69 (SEQ ID NO:69), FIG. 71 (SEQ ID NO:71), FIG. 73 (SEQ ID NO:73), FIG. 75 (SEQ ID NO:75), FIG. 77 (SEQ ID NO:77), FIG. 79 (SEQ ID NO:79), FIG. 81 (SEQ ID NO:81), FIG. 83 (SEQ ID NO:83), FIG. 85 (SEQ ID NO:85), FIG. 87 (SEQ ID NO:87), FIG. 89 (SEQ ID NO:89), FIG. 91 (SEQ ID NO:91), FIG. 93 (SEQ ID NO:93), FIG. 95 (SEQ ID NO:95), FIG. 97 (SEQ ID NO:97), FIG. 99 (SEQ ID NO:99), FIG. 101 (SEQ ID NO:101), FIG. 103 (SEQ ID NO:103), FIG. 105 (SEQ ID NO:105), FIG. 107 (SEQ ID NO:107), FIG. 109 (SEQ ID NO:109), FIG. 111 (SEQ ID NO:11), FIG. 113 (SEQ ID NO:113), FIG. 115 (SEQ ID NO:115), FIG. 117 (SEQ ID NO:117), FIG. 119 (SEQ ID NO:119), FIG. 120 (SEQ ID NO:120), FIG. 122 (SEQ ID NO:122), FIG. 124 (SEQ ID NO:124), FIG. 126 (SEQ ID NO:126), FIG. 128 (SEQ ID NO:128), FIG. 130 (SEQ ID NO:130), FIG. 132 (SEQ ID NO:132), FIG. 134 (SEQ ID NO:134), FIG. 136 (SEQ ID NO:136), FIG. 138 (SEQ ID NO:138), FIG. 140 (SEQ ID NO:140), FIG. 142 (SEQ ID NO:142), FIG. 144 (SEQ ID NO:144), FIG. 146 (SEQ ID NO:146), FIG. 148 (SEQ ID NO:148), FIG. 150 (SEQ ID NO:150), FIG. 152 (SEQ ID NO:152), FIG. 154 (SEQ ID NO:154), FIG. 156 (SEQ ID NO:156), FIG. 158 (SEQ ID NO:158), FIG. 160 (SEQ ID NO:160), FIG. 162 (SEQ ID NO:162), FIG. 164 (SEQ ID NO:164), FIG. 166 (SEQ ID NO:166), FIG. 168 (SEQ ID NO:168), FIG. 170 (SEQ ID NO:170), FIG. 172 (SEQ ID NO:172), FIG. 174 (SEQ ID NO:174), FIG. 176 (SEQ ID NO:176), FIG. 178 (SEQ ID NO:178), FIG. 180 (SEQ ID NO:180), FIG. 182 (SEQ ID NO:182), FIG. 184 (SEQ ID NO:184), FIG. 186 (SEQ ID NO:186), FIG. 188 (SEQ ID NO:188), FIG. 190 (SEQ ID NO:190), FIG. 192 (SEQ ID NO:192), FIG. 194 (SEQ ID NO:194), FIG. 196 (SEQ ID NO:196), FIG. 198 (SEQ ID NO:198), FIG. 200 (SEQ ID NO:200), FIG. 202 (SEQ ID NO:202), FIG. 204 (SEQ ID NO:204), FIG. 206 (SEQ ID NO:206), FIG. 208 (SEQ ID NO:208), FIG. 210 (SEQ ID NO:210), FIG. 212 (SEQ ID NO:212), FIG. 214 (SEQ ID NO:214), FIG. 216 (SEQ ID NO:216), FIG. 218 (SEQ ID NO:218), FIG. 220 (SEQ ID NO:220), FIG. 222 (SEQ ID NO:222), FIG. 224 (SEQ ID NO:224), FIG. 226 (SEQ ID NO:226), FIG. 228 (SEQ ID NO:228), FIG. 230 (SEQ ID NO:231), FIG. 232 (SEQ ID NO:232), FIG. 234 (SEQ ID NO:234), FIG. 236 (SEQ ID NO:236), FIG. 238 (SEQ ID NO:238), FIG. 240 (SEQ ID NO:240), FIG. 242 (SEQ ID NO:242), FIG. 244 (SEQ ID NO:244), FIG. 246 (SEQ ID NO:246), FIG. 248 (SEQ ID NO:248), FIG. 250 (SEQ ID NO:250), FIG. 252 (SEQ ID NO:252), FIG. 253 (SEQ ID NO:253), FIG. 255 (SEQ ID NO:255), FIG. 257 (SEQ ID NO:257), FIG. 259 (SEQ ID NO:259), FIG. 261 (SEQ ID NO:261), FIG. 263 (SEQ ID NO:263), FIG. 265 (SEQ ID NO:265), FIG. 267 (SEQ ID NO:267), FIG. 269 (SEQ ID NO:269), FIG. 271 (SEQ ID NO:271), FIG. 273 (SEQ ID NO:273), FIG. 275 (SEQ ID NO:275), FIG. 277 (SEQ ID NO:277), FIG. 279 (SEQ ID NO:279), FIG. 281 (SEQ ID NO:281), FIG. 283 (SEQ ID NO:283), FIG. 285 (SEQ ID NO:285), FIG. 287 (SEQ ID NO:287), FIG. 289 (SEQ ID NO:289), FIG. 291 (SEQ ID NO:291), FIG. 293 (SEQ ID NO:293), FIG. 295 (SEQ ID NO:295), FIG. 297 (SEQ ID NO:297), FIG. 299 (SEQ ID NO:299), FIG. 301 (SEQ ID NO:301), FIG. 303 (SEQ ID NO:303), FIG. 305 (SEQ ID NO:305), FIG. 307 (SEQ ID NO:307), FIG. 309 (SEQ ID NO:309), FIG. 311 (SEQ ID NO:311), FIG. 313 (SEQ ID NO:313), FIG. 315 (SEQ ID NO:315), FIG. 317 (SEQ ID NO:317), FIG. 319 (SEQ ID NO:319), FIG. 321 (SEQ ID NO:321), FIG. 323 (SEQ ID NO:323), FIG. 325 (SEQ ID NO:325), FIG. 327 (SEQ ID NO:327), FIG. 329 (SEQ ID NO:329), FIG. 331 (SEQ ID NO:331), FIG. 333 (SEQ ID NO:333), FIG. 335 (SEQ ID NO:335), FIG. 337 (SEQ ID NO:337), FIG. 339 (SEQ ID NO:339), FIG. 341 (SEQ ID NO:341), FIG. 343 (SEQ ID NO:343), FIG. 345 (SEQ ID NO:345), FIG. 347 (SEQ ID NO:347), FIG. 349 (SEQ ID NO:349), FIG. 351 (SEQ ID NO:351), FIG. 353 (SEQ ID NO:353), FIG. 355 (SEQ ID NO:355), FIG. 357 (SEQ ID NO:357), FIG. 359 (SEQ ID NO:359), FIG. 361 (SEQ ID NO:361), FIG. 363 (SEQ ID NO:363), FIG. 365 (SEQ ID NO:365), FIG. 367 (SEQ ID NO:367), FIG. 369 (SEQ ID NO:369), FIG. 371 (SEQ ID NO:371), FIG. 373 (SEQ ID NO:373), FIG. 375 (SEQ ID NO:375), FIG. 377 (SEQ ID NO:377), FIG. 379 (SEQ ID NO:379), FIG. 381 (SEQ ID NO:381), FIG. 383 (SEQ ID NO:383), FIG. 385 (SEQ ID NO:385), FIG. 387 (SEQ ID NO:387), FIG. 389 (SEQ ID NO:389), FIG. 391 (SEQ ID NO:391), FIG. 393 (SEQ ID NO:393), FIG. 395 (SEQ ID NO:395), FIG. 397 (SEQ ID NO:397), FIG. 399 (SEQ ID NO:399), FIG. 401 (SEQ ID NO:401), FIG. 403 (SEQ ID NO:403), FIG. 405 (SEQ ID NO:405), FIG. 407 (SEQ ID NO:407), FIG. 409 (SEQ ID NO:409), FIG. 411 (SEQ ID NO:411), FIG. 413 (SEQ ID NO:413), FIG. 415 (SEQ ID NO:415), FIG. 417 (SEQ ID NO:417), FIG. 419 (SEQ ID NO:419), FIG. 421 (SEQ ID NO:421), FIG. 423 (SEQ ID NO:423), FIG. 425 (SEQ ID NO:425), FIG. 427 (SEQ ID NO:427), FIG. 429 (SEQ ID NO:429), FIG. 431 (SEQ ID NO:431), FIG. 433 (SEQ ID NO:433), FIG. 435 (SEQ ID NO:435), FIG. 437 (SEQ ID NO:437), FIG. 439 (SEQ ID NO:439), FIG. 441 (SEQ ID NO:441), FIG. 443 (SEQ ID NO:443), FIG. 445 (SEQ ID NO:445), FIG. 447 (SEQ ID NO:447), FIG. 449 (SEQ ID NO:449), FIG. 451 (SEQ ID NO:451), FIG. 453 (SEQ ID NO:453), FIG. 455 (SEQ ID NO:455), FIG. 457 (SEQ ID NO:457), FIG. 459 (SEQ ID NO:459), FIG. 461 (SEQ ID NO:461), FIG. 463 (SEQ ID NO:463), FIG. 465 (SEQ ID NO:465), FIG. 467 (SEQ ID NO:467), FIG. 469 (SEQ ID NO:469), FIG. 471 (SEQ ID NO:471), FIG. 473 (SEQ ID NO:473), FIG. 475 (SEQ ID NO:475), FIG. 477 (SEQ ID NO:477), FIG. 479 (SEQ ID NO:479), FIG. 481 (SEQ ID NO:481), FIG. 483 (SEQ ID NO:483), FIG. 485 (SEQ ID NO:485), FIG. 487 (SEQ ID NO:487), FIG. 489 (SEQ ID NO:489), FIG. 491 (SEQ ID NO:491), FIG. 493 (SEQ ID NO:493), FIG. 495 (SEQ ID NO:495), FIG. 497 (SEQ ID NO:497), FIG. 499 (SEQ ID NO:499), FIG. 501 (SEQ ID NO:501), FIG. 503 (SEQ ID NO:503), FIG. 505 (SEQ ID NO:505), FIG. 507 (SEQ ID NO:507), FIG. 509 (SEQ ID NO:509), FIG. 511 (SEQ ID NO:511), FIG. 513 (SEQ ID NO:513), FIG. 515 (SEQ ID NO:515), FIG. 517 (SEQ ID NO:517), FIG. 519 (SEQ ID NO:519), FIG. 521 (SEQ ID NO:521), FIG. 523 (SEQ ID NO:523), FIG. 525 (SEQ ID NO:525), FIG. 527 (SEQ ID NO:527), FIG. 529 (SEQ ID NO:529), FIG. 531 (SEQ ID NO:531), FIG. 533 (SEQ ID NO:533), FIG. 535 (SEQ ID NO:535), FIG. 537 (SEQ ID NO:537), FIG. 539 (SEQ ID NO:539), FIG. 541 (SEQ ID NO:541), FIG. 543 (SEQ ID NO:543), FIG. 545 (SEQ ID NO:545), FIG. 547 (SEQ ID NO:547), FIG. 548 (SEQ ID NO:548), FIG. 550 (SEQ ID NO:550), FIG. 552 (SEQ ID NO:552), FIG. 554 (SEQ ID NO:554), FIG. 556 (SEQ ID NO:556), FIG. 558 (SEQ ID NO:558), FIG. 560 (SEQ ID NO:560), FIG. 562 (SEQ ID NO:562), FIG. 564 (SEQ ID NO:564), FIG. 566 (SEQ ID NO:566), FIG. 568 (SEQ ID NO:568), FIG. 570 (SEQ ID NO:570), FIG. 572 (SEQ ID NO:572), FIG. 574 (SEQ ID NO:574), FIG. 576 (SEQ ID NO:576), FIG. 578 (SEQ ID NO:578), FIG. 580 (SEQ ID NO:580), FIG. 582 (SEQ ID NO:582), FIG. 584 (SEQ ID NO:584), FIG. 586 (SEQ ID NO:586), FIG. 588 (SEQ ID NO:588), FIG. 590 (SEQ ID NO:590), FIG. 592 (SEQ ID NO:592), FIG. 594 (SEQ ID NO:594), FIG. 596 (SEQ ID NO:596), FIG. 598 (SEQ ID NO:598), FIG. 600 (SEQ ID NO:600), FIG. 602 (SEQ ID NO:602), FIG. 604 (SEQ ID NO:604), FIG. 606 (SEQ ID NO:606), FIG. 608 (SEQ ID NO:608), FIG. 610 (SEQ ID NO:610), FIG. 612 (SEQ ID NO:612), FIG. 614 (SEQ ID NO:614), FIG. 616 (SEQ ID NO:616), FIG. 618 (SEQ ID NO:618), FIG. 620 (SEQ ID NO:620), FIG. 622 (SEQ ID NO:622), FIG. 624 (SEQ ID NO:624), FIG. 626 (SEQ ID NO:626), FIG. 628 (SEQ ID NO:628), FIG. 630 (SEQ ID NO:630), FIG. 632 (SEQ ID NO:632), FIG. 634 (SEQ ID NO:634), FIG. 636 (SEQ ID NO:636), FIG. 638 (SEQ ID NO:638), FIG. 640 (SEQ ID NO:640), FIG. 642 (SEQ ID NO:642), FIG. 644 (SEQ ID NO:644), FIG. 646 (SEQ ID NO:646), FIG. 648 (SEQ ID NO:648), FIG. 650 (SEQ ID NO:650), FIG. 652 (SEQ ID NO:652), FIG. 654 (SEQ ID NO:654), FIG. 656 (SEQ ID NO:656), FIG. 658 (SEQ ID NO:658), FIG. 660 (SEQ ID NO:660), FIG. 662 (SEQ ID NO:662), FIG. 664 (SEQ ID NO:664), FIG. 666 (SEQ ID NO:666), FIG. 668 (SEQ ID NO:668), FIG. 670 (SEQ ID NO:670), FIG. 672 (SEQ ID NO:672), FIG. 674 (SEQ ID NO:674), FIG. 676 (SEQ ID NO:676), FIG. 678 (SEQ ID NO:678), FIG. 680 (SEQ ID NO:680), FIG. 682 (SEQ ID NO:682), FIG. 684 (SEQ ID NO:684), FIG. 686 (SEQ ID NO:686), FIG. 688 (SEQ ID NO:688), FIG. 690 (SEQ ID NO:690), FIG. 692 (SEQ ID NO:692), FIG. 694 (SEQ ID NO:694), FIG. 696 (SEQ ID NO:696), FIG. 698 (SEQ ID NO:698), FIG. 700 (SEQ ID NO:700), FIG. 702 (SEQ ID NO:702), FIG. 704 (SEQ ID NO:704), FIG. 706 (SEQ ID NO:706), FIG. 708 (SEQ ID NO:708), FIG. 710 (SEQ ID NO:710), FIG. 712 (SEQ ID NO:712), FIG. 714 (SEQ ID NO:714), FIG. 716 (SEQ ID NO:716), FIG. 718 (SEQ ID NO:718), FIG. 720 (SEQ ID NO:720), FIG. 722 (SEQ ID NO:722), FIG. 724 (SEQ ID NO:724), FIG. 726 (SEQ ID NO:726), FIG. 728 (SEQ ID NO:728), FIG. 730 (SEQ ID NO:730), FIG. 732 (SEQ ID NO:732), FIG. 734 (SEQ ID NO:734), FIG. 736 (SEQ ID NO:736), FIG. 738 (SEQ ID NO:738), FIG. 740 (SEQ ID NO:740), FIG. 742 (SEQ ID NO:742), FIG. 744 (SEQ ID NO:744), FIG. 746 (SEQ ID NO:746), FIG. 748 (SEQ ID NO:748), FIG. 750 (SEQ ID NO:750), FIG. 752 (SEQ ID NO:752), FIG. 754 (SEQ ID NO:754), FIG. 756 (SEQ ID NO:756), FIG. 758 (SEQ ID NO:758), FIG. 760 (SEQ ID NO:760), FIG. 762 (SEQ ID NO:762), FIG. 764 (SEQ ID NO:764), FIG. 766 (SEQ ID NO:766), FIG. 768 (SEQ ID NO:768), FIG. 770 (SEQ ID NO:770), FIG. 772 (SEQ ID NO:772), FIG. 774 (SEQ ID NO:774), FIG. 776 (SEQ ID NO:776), FIG. 778 (SEQ ID NO:778), FIG. 780 (SEQ ID NO:780), FIG. 782 (SEQ ID NO:782), FIG. 784 (SEQ ID NO:784), FIG. 786 (SEQ ID NO:786), FIG. 788 (SEQ ID NO:788), FIG. 790 (SEQ ID NO:790), FIG. 792 (SEQ ID NO:792), FIG. 794 (SEQ ID NO:794), FIG. 796 (SEQ ID NO:796), FIG. 798 (SEQ ID NO:798), FIG. 800 (SEQ ID NO:800), FIG. 802 (SEQ ID NO:802), FIG. 804 (SEQ ID NO:804), FIG. 806 (SEQ ID NO:806), FIG. 808 (SEQ ID NO:808), FIG. 810A-B (SEQ ID NO:810), FIG. 812 (SEQ ID NO:812), FIG. 814 (SEQ ID NO:814), FIG. 816 (SEQ ID NO:816), FIG. 818 (SEQ ID NO:818), FIG. 820 (SEQ ID NO:820), FIG. 822 (SEQ ID NO:822), FIG. 824 (SEQ ID NO:824), FIG. 826 (SEQ ID NO:826), FIG. 828 (SEQ ID NO:828), FIG. 830 (SEQ ID NO:830), FIG. 832 (SEQ ID NO:832), FIG. 834 (SEQ ID NO:834), FIG. 836 (SEQ ID NO:836), FIG. 838 (SEQ ID NO:838), FIG. 840 (SEQ ID NO:840), FIG. 842 (SEQ ID NO:842), FIG. 844 (SEQ ID NO:844), FIG. 846 (SEQ ID NO:846), FIG. 848 (SEQ ID NO:848), FIG. 850 (SEQ ID NO:850), FIG. 852 (SEQ ID NO:852), FIG. 854 (SEQ ID NO:854), FIG. 856 (SEQ ID NO:856), FIG. 857A-B (SEQ ID NO:857), FIG. 859A-B (SEQ ID NO:859), FIG. 861 (SEQ ID NO:861), FIG. 863 (SEQ ID NO:863), FIG. 865 (SEQ ID NO:865), FIG. 867 (SEQ ID NO:867), FIG. 869 (SEQ ID NO:869), FIG. 871 (SEQ ID NO:871), FIG. 873 (SEQ ID NO:873), FIG. 875 (SEQ ID NO:875), FIG. 877 (SEQ ID NO:877), FIG. 879 (SEQ ID NO:879), FIG. 881A-B (SEQ ID NO:881), FIG. 883 (SEQ ID NO:883), FIG. 885 (SEQ ID NO:885), FIG. 887 (SEQ ID NO:887), FIG. 889 (SEQ ID NO:889), FIG. 891 (SEQ ID NO:891), FIG. 893 (SEQ ID NO:893), FIG. 895 (SEQ ID NO:895), FIG. 897 (SEQ ID NO:897), FIG. 899 (SEQ ID NO:899), FIG. 901 (SEQ ID NO:901), FIG. 903 (SEQ ID NO:903), FIG. 905A-C (SEQ ID NO:905), FIG. 907 (SEQ ID NO:907), FIG. 909 (SEQ ID NO:909), FIG. 911 (SEQ ID NO:911), FIG. 913 (SEQ ID NO:913), FIG. 915 (SEQ ID NO:915), FIG. 917 (SEQ ID NO:917), FIG. 919A-B (SEQ ID NO:919), FIG. 921 (SEQ ID NO:921), FIG. 923 (SEQ ID NO:923), FIG. 925 (SEQ ID NO:925), FIG. 927 (SEQ ID NO:927), FIG. 929 (SEQ ID NO:929), FIG. 931 (SEQ ID NO:931), FIG. 933A-B (SEQ ID NO:933), FIG. 935 (SEQ ID NO:935), FIG. 937A-B (SEQ ID NO:937), FIG. 939A-B (SEQ ID NO:939), FIG. 941A-B (SEQ ID NO:941), FIG. 942A-B (SEQ ID NO:942), FIG. 944 (SEQ ID NO:944), FIG. 946 (SEQ ID NO:946), FIG. 948A-C (SEQ ID NO:948), FIG. 950 (SEQ ID NO:950), FIG. 952 (SEQ ID NO:952), FIG. 954A-B (SEQ ID NO:954), FIG. 956A-B (SEQ ID NO:956), FIG. 957A-B (SEQ ID NO:957), FIG. 959 (SEQ ID NO:959), FIG. 961 (SEQ ID NO:961), FIG. 962 (SEQ ID NO:962), FIG. 964 (SEQ ID NO:964), FIG. 966 (SEQ ID NO:966), FIG. 967 (SEQ ID NO:967), FIG. 969A-B (SEQ ID NO:969), FIG. 971A-B (SEQ ID NO:971), FIG. 973 (SEQ ID NO:973), FIG. 975 (SEQ ID NO:975), FIG. 977A-B (SEQ ID NO:977), FIG. 979 (SEQ ID NO:979), FIG. 981 (SEQ ID NO:981), FIG. 982 (SEQ ID NO:982), FIG. 984 (SEQ ID NO:984), FIG. 986 (SEQ ID NO:986), FIG. 988 (SEQ ID NO:988), FIG. 989A-D (SEQ ID NO:989), FIG. 991 (SEQ ID NO:991), FIG. 993A-C (SEQ ID NO:993), FIG. 995 (SEQ ID NO:995), FIG. 997 (SEQ ID NO:997), FIG. 999A-B (SEQ ID NO:999), FIG. 1001 (SEQ ID NO:1001), FIG. 1003A-B (SEQ ID NO:1003), FIG. 1004A-B (SEQ ID NO:1004), FIG. 1005A-B (SEQ ID NO:1005), FIG. 1006A-B (SEQ ID NO:1006), FIG. 1008 (SEQ ID NO:1008), FIG. 1010A-B (SEQ ID NO:1010), FIG. 1012 (SEQ ID NO:1012), FIG. 1014 (SEQ ID NO:1014), FIG. 1016A-B (SEQ ID NO:1016), FIG. 1018 (SEQ ID NO:1018), FIG. 1020A-B (SEQ ID NO:1020), FIG. 1022 (SEQ ID NO:1022), FIG. 1024A-C (SEQ ID NO:1024), FIG. 1026A-B (SEQ ID NO:1026), FIG. 1027 (SEQ ID NO:1027), FIG. 1028A-B (SEQ ID NO:1028), FIG. 1029 (SEQ ID NO:1029), FIG. 1031 (SEQ ID NO:1031), FIG. 1032 (SEQ ID NO:1032), FIG. 1034A-C (SEQ ID NO:1034), FIG. 1036A-B (SEQ ID NO:1036), FIG. A-B 1038 (SEQ ID NO:1038), FIG. 1040 (SEQ ID NO:1040), FIG. 1042A-B (SEQ ID NO:1042), FIG. 1044 A-B (SEQ ID NO:1044), FIG. 1045 (SEQ ID NO:1045), FIG. 1047 (SEQ ID NO:1047), FIG. 1049 (SEQ ID NO:1049), FIG. 1051 (SEQ ID NO:1051), FIG. 1052A-B (SEQ ID NO:1052), FIG. 1054 (SEQ ID NO:1054), FIG. 1056 (SEQ ID NO:1056), FIG. 1058 (SEQ ID NO:1058), FIG. 1059 (SEQ ID NO:1059), FIG. 1061 (SEQ ID NO:1061), FIG. 1063 (SEQ ID NO:1063), FIG. 1065 (SEQ ID NO:1065), FIG. 1067 (SEQ ID NO:1067), FIG. 1069A-B (SEQ ID NO:1069), FIG. 1071 (SEQ ID NO:1071), FIG. 1073A-B (SEQ ID NO:1073), FIG. 1075 (SEQ ID NO:1075), FIG. 1077 (SEQ ID NO:1077), FIG. 1079 (SEQ ID NO:1079), FIG. 1081 (SEQ ID NO:1081), FIG. 1083A-B (SEQ ID NO:1083), FIG. 1085 (SEQ ID NO:1085), FIG. 1087 (SEQ ID NO:1087), FIG. 1089 (SEQ ID NO:1089), FIG. 1091 (SEQ ID NO:1091), FIG. 1093A-B (SEQ ID NO:1093), FIG. 1095 (SEQ ID NO:1095), FIG. 1097 (SEQ ID NO:1097), FIG. 1099 (SEQ ID NO:1099), FIG. 1100 (SEQ ID NO:100), FIG. 1102A-B (SEQ ID NO:1102), FIG. 1104A-B (SEQ ID NO:1104), FIG. 1106 (SEQ ID NO:1106), FIG. 1108 (SEQ ID NO:1108), FIG. 1110 (SEQ ID NO:1110), FIG. 1112 (SEQ ID NO:1112), FIG. 1114A-B (SEQ ID NO:1114), FIG. 1115 (SEQ ID NO:1115), FIG. 1117A-B (SEQ ID NO:1117), FIG. 1119 (SEQ ID NO:1119), FIG. 1121 (SEQ ID NO:1121), FIG. 1122 (SEQ ID NO:1122), FIG. 1124 (SEQ ID NO:1124), FIG. 1126 (SEQ ID NO:1126), FIG. 1127 (SEQ ID NO:1127), FIG. 1129 (SEQ ID NO:1129), FIG. 1131 (SEQ ID NO:1131), FIG. 1133 (SEQ ID NO:1133), FIG. 1135 (SEQ ID NO:1135), FIG. 1137A-B (SEQ ID NO:1137), FIG. 1139 (SEQ ID NO:1139), FIG. 1140 (SEQ ID NO:1140), FIG. 1141 (SEQ ID NO:1141), FIG. 1143A-C (SEQ ID NO:1143), FIG. 1145A-B (SEQ ID NO:1145), FIG. 1147 (SEQ ID NO:1147), FIG. 1149 (SEQ ID NO:1149), FIG. 1151 (SEQ ID NO:1151), FIG. 1153 (SEQ ID NO:1153), FIG. 1155A-B (SEQ ID NO:1155), FIG. 1157 (SEQ ID NO:1157), FIG. 1159 (SEQ ID NO:1159), FIG. 1161 (SEQ ID NO:1161), FIG. 1163 (SEQ ID NO:1163), FIG. 1165 (SEQ ID NO:1165), FIG. 1167 (SEQ ID NO:1167), FIG. 1169 (SEQ ID NO:1169), FIG. 1171 (SEQ ID NO:1171), FIG. 1173 (SEQ ID NO:1173), FIG. 1175 (SEQ ID NO:1175), FIG. 1177 (SEQ ID NO:1177), FIG. 1179 (SEQ ID NO:1179), FIG. 1181 (SEQ ID NO:1181), FIG. 1183 (SEQ ID NO:1183), FIG. 1185 (SEQ ID NO:1185), FIG. 1187 (SEQ ID NO:), FIG. 1189 (SEQ ID NO:1189), FIG. 1191 (SEQ ID NO:1191), FIG. 1193 (SEQ ID NO:1193), FIG. 1195 (SEQ ID NO:1195), FIG. 1197 (SEQ ID NO:1197), FIG. 1199 (SEQ ID NO:1199), FIG. 1201 (SEQ ID NO:1201), FIG. 1203 (SEQ ID NO:1203), FIG. 1205 (SEQ ID NO:1205), FIG. 1207 (SEQ ID NO:1207), FIG. 1209 (SEQ ID NO:1209), FIG. 1211 (SEQ ID NO:1211), FIG. 1213 (SEQ ID NO:1213), FIG. 1215 (SEQ ID NO:1215), FIG. 1217 (SEQ ID NO:1217), FIG. 1219 (SEQ ID NO:1219), FIG. 1221 (SEQ ID NO:1221), FIG. 1223A-B (SEQ ID NO:1223), FIG. 1225 (SEQ ID NO:1225), FIG. 1227 (SEQ ID NO:1227), FIG. 1229 (SEQ ID NO:1229), FIG. 1231 (SEQ ID NO:1231), FIG. 1233 (SEQ ID NO:1233), FIG. 1235 (SEQ ID NO:1235), FIG. 1237 (SEQ ID NO:1237), FIG. 1239 (SEQ ID NO:1239), FIG. 1241 (SEQ ID NO:1241), FIG. 1243 (SEQ ID NO:1243), FIG. 1245 (SEQ ID NO:1245), FIG. 1247 (SEQ ID NO:1247), FIG. 1249 (SEQ ID NO:1249), FIG. 1251 (SEQ ID NO:1251), FIG. 1253 (SEQ ID NO:1253), FIG. 1255 (SEQ ID NO:1255), FIG. 1257 (SEQ ID NO:1257), FIG. 1259 (SEQ ID NO:1259), FIG. 1261 (SEQ ID NO:1261), FIG. 1263 (SEQ ID NO:1263), FIG. 1265 (SEQ ID NO:1265), FIG. 1267 (SEQ ID NO:1267), FIG. 1269 (SEQ ID NO:1269), FIG. 1271 (SEQ ID NO:1271), FIG. 1273 (SEQ ID NO:1273), FIG. 1275 (SEQ ID NO:1275), FIG. 1277A-C (SEQ ID NO:1277), FIG. 1279 (SEQ ID NO:1279), FIG. 1281 (SEQ ID NO:1281), FIG. 1283A-B (SEQ ID NO:1283), FIG. 1285 (SEQ ID NO:1285), FIG. 1287A-B (SEQ ID NO:1287), FIG. 1289 (SEQ ID NO:1289), FIG. 1291 (SEQ ID NO:1291), FIG. 1293 (SEQ ID NO:1293), FIG. 1295 (SEQ ID NO:1295), FIG. 1297A-B (SEQ ID NO:1297), FIG. 1299 (SEQ ID NO:1299), FIG. 1301A-B (SEQ ID NO:1301), FIG. 1303 (SEQ ID NO:1303), FIG. 1305 (SEQ ID NO:1305), FIG. 1307 (SEQ ID NO:1307), FIG. 1309 (SEQ ID NO:1309), FIG. 1311 (SEQ ID NO:1311), FIG. 1313 (SEQ ID NO:1313), FIG. 1315 (SEQ ID NO:1315), FIG. 1317 (SEQ ID NO:1317), FIG. 1319 (SEQ ID NO:1319), FIG. 1321 A-B (SEQ ID NO:1321), FIG. 1323 (SEQ ID NO:1323), FIG. 1325 (SEQ ID NO:1325), FIG. 1327 (SEQ ID NO:1327), FIG. 1329 (SEQ ID NO:1329), FIG. 1331 (SEQ ID NO:1331), FIG. 1333 (SEQ ID NO:1333), FIG. 1335 (SEQ ID NO:1335), FIG. 1337 (SEQ ID NO:1337), FIG. 1339 (SEQ ID NO:1339), FIG. 1341 (SEQ ID NO:1341), FIG. 1343 (SEQ ID NO:1343), FIG. 1345 (SEQ ID NO:1345), FIG. 1347 (SEQ ID NO:1347), FIG. 1349 A-B (SEQ ID NO:1349), FIG. 1351 (SEQ ID NO:1351), FIG. 1353 (SEQ ID NO:1353), FIG. 1355 A-B (SEQ ID NO:1355), FIG. 1357 (SEQ ID NO:1357), FIG. 1359 (SEQ ID NO:1359), FIG. 1360 (SEQ ID NO:1360), FIG. 1362 (SEQ ID NO:1362), FIG. 1364 (SEQ ID NO:1364), FIG. 1366 (SEQ ID NO:1366), FIG. 1368 (SEQ ID NO:1368), FIG. 1370 (SEQ ID NO:1370), FIG. 1372 (SEQ ID NO:1372), FIG. 1374 (SEQ ID NO:1374), FIG. 1376 (SEQ ID NO:1376), FIG. 1378 (SEQ ID NO:1378), FIG. 1380 A-B (SEQ ID NO:1380), FIG. 1382 (SEQ ID NO:1382), FIG. 1384 (SEQ ID NO:1384), FIG. 1386 (SEQ ID NO:1386), FIG. 1388 (SEQ ID NO:1388), FIG. 1390 A-B (SEQ ID NO:1390), FIG. 1392 (SEQ ID NO:1392), FIG. 1394 (SEQ ID NO:1394), FIG. 1396 (SEQ ID NO:1396), FIG. 1398 A-B (SEQ ID NO:1398), FIG. 1400 (SEQ ID NO:1400), FIG. 1402 (SEQ ID NO:1402), FIG. 1404 (SEQ ID NO:1404), FIG. 1406 (SEQ ID NO:1406), FIG. 1408 (SEQ ID NO:1408), FIG. 1410 (SEQ ID NO:1410), FIG. 1412 (SEQ ID NO:1412), FIG. 1414 (SEQ ID NO:1414), FIG. 1416 (SEQ ID NO:1416), FIG. 1418 (SEQ ID NO:1418), FIG. 1420 (SEQ ID NO:1420), FIG. 1422 (SEQ ID NO:1422), FIG. 1424 (SEQ ID NO:1424), FIG. 1426 (SEQ ID NO:1426), FIG. 1428 (SEQ ID NO:1428), FIG. 1430 (SEQ ID NO:1430), FIG. 1432 A-B (SEQ ID NO:1432), FIG. 1434 (SEQ ID NO:1434), FIG. 1436 A-B (SEQ ID NO:1436), FIG. 1438 (SEQ ID NO:1438), FIG. 1440 (SEQ ID NO:1440), FIG. 1442 A-B (SEQ ID NO:1442), FIG. 1444 A-B (SEQ ID NO:1444), FIG. 1446 (SEQ ID NO:1446), FIG. 1448 (SEQ ID NO:1448), FIG. 1450 (SEQ ID NO:1450), FIG. 1452 (SEQ ID NO:1452), FIG. 1454 (SEQ ID NO:1454), FIG. 1456 (SEQ ID NO:1456), FIG. 1458 (SEQ ID NO:1458), FIG. 1460 (SEQ ID NO:1460), FIG. 1462 (SEQ ID NO:1462), FIG. 1464 (SEQ ID NO:1464), FIG. 1466 (SEQ ID NO:1466), FIG. 1468 (SEQ ID NO:1468), FIG. 1470 (SEQ ID NO:1470), FIG. 1472 (SEQ ID NO:1472), FIG. 1474 (SEQ ID NO:1474), FIG. 1476 (SEQ ID NO:1476), FIG. 1478 (SEQ ID NO:1478), FIG. 1480 (SEQ ID NO:1480), FIG. 1482 (SEQ ID NO:1482), FIG. 1484 (SEQ ID NO:1484), FIG. 1486 (SEQ ID NO:1486), FIG. 1488A-B (SEQ ID NO:1488), FIG. 1490 (SEQ ID NO:1490), FIG. 1492 (SEQ ID NO:1492), FIG. 1494 (SEQ ID NO:1494), FIG. 1496 (SEQ ID NO:1496), FIG. 1498 (SEQ ID NO:1498), FIG. 1500 (SEQ ID NO:1500), FIG. 1502 (SEQ ID NO:1502), FIG. 1504 (SEQ ID NO:1504), FIG. 1506 (SEQ ID NO:1506), FIG. 1508 (SEQ ID NO:1508), FIG. 1510 (SEQ ID NO:1510), FIG. 1512 (SEQ ID NO:1512), FIG. 1514 (SEQ ID NO:1514), FIG. 1516 (SEQ ID NO:1516), FIG. 1518 (SEQ ID NO:1518), FIG. 1520 (SEQ ID NO:1520), FIG. 1522 (SEQ ID NO:1522), FIG. 1524 (SEQ ID NO:1524), FIG. 1526 (SEQ ID NO:1526), FIG. 1528 (SEQ ID NO:1528), FIG. 1530 (SEQ ID NO:1530), FIG. 1532 (SEQ ID NO:1532), FIG. 1534 (SEQ ID NO:1534), FIG. 1536 (SEQ ID NO:1536), FIG. 1538 (SEQ ID NO:1538), FIG. 1540 (SEQ ID NO:1540), FIG. 1542 (SEQ ID NO:1542), FIG. 1544 (SEQ ID NO:1544), FIG. 1546 (SEQ ID NO:1546), FIG. 1548 (SEQ ID NO:1548), FIG. 1550 (SEQ ID NO:1550), FIG. 1552 (SEQ ID NO:1552), FIG. 1554 (SEQ ID NO:1554), FIG. 1556 (SEQ ID NO:1556), FIG. 1558 A-B (SEQ ID NO:1558), FIG. 1560 (SEQ ID NO:1560), FIG. 1562 (SEQ ID NO:1562), FIG. 1564 (SEQ ID NO:1564), FIG. 1566 (SEQ ID NO:1566), FIG. 1568 (SEQ ID NO:1568), FIG. 1570 A-B (SEQ ID NO:1570), FIG. 1572A-C (SEQ ID NO:1572), FIG. 1574 (SEQ ID NO:1574), FIG. 1576 (SEQ ID NO:1576), FIG. 1577 (SEQ ID NO:1577), FIG. 1579 (SEQ ID NO:1579), FIG. 1580 (SEQ ID NO:1580), FIG. 1582 A-B (SEQ ID NO:1582), FIG. 1584 (SEQ ID NO:1584), FIG. 1586 (SEQ ID NO:1586), FIG. 1588 A-B (SEQ ID NO:1588), FIG. 1590 (SEQ ID NO:1590), FIG. 1591 (SEQ ID NO:1591), FIG. 1593 (SEQ ID NO:1593), FIG. 1595 (SEQ ID NO:1595), FIG. 1597 (SEQ ID NO:1597), FIG. 1599A-D (SEQ ID NO:1599), FIG. 1601A-E (SEQ ID NO:1601), FIG. 1603 A-B (SEQ ID NO:1603), FIG. 1605 A-B (SEQ ID NO:1605), FIG. 1607 (SEQ ID NO:1607), FIG. 1608 (SEQ ID NO:1608), FIG. 1610 (SEQ ID NO:1610), FIG. 1612 (SEQ ID NO:1612), FIG. 1613 A-B (SEQ ID NO:1613), FIG. 1615 (SEQ ID NO:1615), FIG. 1616 (SEQ ID NO:1616), FIG. 1618 (SEQ ID NO:1618), FIG. 1620 A-B (SEQ ID NO:1620), FIG. 1622 (SEQ ID NO:1622), FIG. 1624 (SEQ ID NO:1624), FIG. 1625 (SEQ ID NO:1625), FIG. 1627 (SEQ ID NO:1627), FIG. 1629 (SEQ ID NO:1629), FIG. 1631 (SEQ ID NO:1631), FIG. 1633 (SEQ ID NO:1633), FIG. 1634 (SEQ ID NO:1634), FIG. 1635 (SEQ ID NO:1635), FIG. 1637 (SEQ ID NO:1637), FIG. 1638 (SEQ ID NO:1638), FIG. 1640 (SEQ ID NO:1640), FIG. 1641 (SEQ ID NO:1641), FIG. 1642 (SEQ ID NO:1642), FIG. 1643 (SEQ ID NO:1643), FIG. 1645 (SEQ ID NO:1645), FIG. 1647 A-B (SEQ ID NO:1647), FIG. 1649 (SEQ ID NO:1649), FIG. 1651 (SEQ ID NO:1651), FIG. 1653 (SEQ ID NO:1653), FIG. 1655 (SEQ ID NO:1655), FIG. 1657 (SEQ ID NO:1657), FIG. 1659 (SEQ ID NO:1659), FIG. 1661 (SEQ ID NO:1661), FIG. 1663 (SEQ ID NO:1663), FIG. 1665 (SEQ ID NO:1665), FIG. 1667 (SEQ ID NO:1667), FIG. 1669 (SEQ ID NO:1669), FIG. 1671 (SEQ ID NO:1671), FIG. 1673 (SEQ ID NO:1673), FIG. 1675 (SEQ ID NO:1675), FIG. 1677 (SEQ ID NO:1677), FIG. 1679 A-B (SEQ ID NO:1679), FIG. 1681 (SEQ ID NO:1681), FIG. 1682A-B (SEQ ID NO:1682), FIG. 1684 A-B (SEQ ID NO:1684), FIG. 1686 A-B (SEQ ID NO:1686), FIG. 1688 (SEQ ID NO:1688), FIG. 1690 (SEQ ID NO:1690), FIG. 1691 (SEQ ID NO:1691), FIG. 1693 (SEQ ID NO:1693), FIG. 1695 (SEQ ID NO:1695), FIG. 1697 (SEQ ID NO:1697), FIG. 1699 (SEQ ID NO:1699), FIG. 1700 (SEQ ID NO:1700), FIG. 1702 (SEQ ID NO:1702), FIG. 1704 (SEQ ID NO:1704), FIG. 1706 (SEQ ID NO:1706), FIG. 1708A-C (SEQ ID NO:1708), FIG. 1710 (SEQ ID NO:1710), FIG. 1712 (SEQ ID NO:1712), FIG. 1714 (SEQ ID NO:1714), FIG. 1716 (SEQ ID NO:1716), FIG. 1718 (SEQ ID NO:1718), FIG. 1720A-C (SEQ ID NO:1720), FIG. 1722 (SEQ ID NO:1722), FIG. 1724A-B (SEQ ID NO:1724), FIG. 1726 (SEQ ID NO:1726), FIG. 1728A-B (SEQ ID NO:1728), FIG. 1730 (SEQ ID NO:1730), FIG. 1731 (SEQ ID NO:1731), FIG. 1733 (SEQ ID NO:1733), FIG. 1735 (SEQ ID NO:1735), FIG. 1737 (SEQ ID NO:1737), FIG. 1739 (SEQ ID NO:1739), FIG. 1740 (SEQ ID NO:1740), FIG. 1742 (SEQ ID NO:1742), FIG. 1744 (SEQ ID NO:1744), FIG. 1746 (SEQ ID NO:1746), FIG. 1748 (SEQ ID NO:1748), FIG. 1750 (SEQ ID NO:1750), FIG. 1752A-B (SEQ ID NO:1752), FIG. 1754 (SEQ ID NO:1754), FIG. 1756 (SEQ ID NO:1756), FIG. 1758 (SEQ ID NO:1758), FIG. 1759 (SEQ ID NO:1759), FIG. 1761 (SEQ ID NO:1761), FIG. 1763 (SEQ ID NO:1763), FIG. 1765 (SEQ ID NO:1765), FIG. 1767 (SEQ ID NO:1767), FIG. 1768 (SEQ ID NO:1768), FIG. 1770 (SEQ ID NO:1770), FIG. 1772 (SEQ ID NO:1772), FIG. 1774 (SEQ ID NO:1774), FIG. 1776 (SEQ ID NO:1776), FIG. 1778 (SEQ ID NO:1778), FIG. 1780 (SEQ ID NO:1780), FIG. 1782 (SEQ ID NO:1782), FIG. 1784 (SEQ ID NO:1784), FIG. 1786 (SEQ ID NO:1786), FIG. 1788 (SEQ ID NO:1788), FIG. 1790 (SEQ ID NO:1790), FIG. 1791 (SEQ ID NO:1791), FIG. 1793 (SEQ ID NO:1793), FIG. 1794 (SEQ ID NO:1794), FIG. 1796 (SEQ ID NO:1796), FIG. 1798 (SEQ ID NO:1798), FIG. 1800 (SEQ ID NO:1800), FIG. 1801 (SEQ ID NO:1801), FIG. 1803 (SEQ ID NO:1803), FIG. 1804 (SEQ ID NO:1804), FIG. 1806 (SEQ ID NO:1806), FIG. 1808 (SEQ ID NO:1808), FIG. 1810 (SEQ ID NO:1810), FIG. 1812A-B (SEQ ID NO:1812), FIG. 1814 (SEQ ID NO:1814), FIG. 1816 (SEQ ID NO:1816), FIG. 1818 (SEQ ID NO:1818), FIG. 1820 (SEQ ID NO:1820), FIG. 1822 (SEQ ID NO:1822), FIG. 1824A-B (SEQ ID NO:1824), FIG. 1826 (SEQ ID NO:1826), FIG. 1828 (SEQ ID NO:1828), FIG. 1830 (SEQ ID NO:1830), FIG. 1832 (SEQ ID NO:1832), FIG. 1834 (SEQ ID NO:1834), FIG. 1835 (SEQ ID NO:1835), FIG. 1837 (SEQ ID NO:1837), FIG. 1839 (SEQ ID NO:1839), FIG. 1840 (SEQ ID NO:1840), FIG. 1842 (SEQ ID NO:1842), FIG. 1844A-B (SEQ ID NO:1844), FIG. 1846 (SEQ ID NO:1846), FIG. 1848A-B (SEQ ID NO:1848), FIG. 1850 (SEQ ID NO:1850), FIG. 1852 (SEQ ID NO:1852), FIG. 1853A-B (SEQ ID NO:1853), FIG. 1855 (SEQ ID NO:1855), FIG. 1857 (SEQ ID NO:1857), FIG. 1859 (SEQ ID NO:1859), FIG. 1861 (SEQ ID NO:1861), FIG. 1862A-B (SEQ ID NO:1862), FIG. 1864 (SEQ ID NO:1864), FIG. 1866 (SEQ ID NO:1866), FIG. 1868A-B (SEQ ID NO:1868), FIG. 1870 (SEQ ID NO:1870), FIG. 1872 (SEQ ID NO:1872), FIG. 1874 (SEQ ID NO:1874), FIG. 1876 (SEQ ID NO:1876), FIG. 1878 (SEQ ID NO:1878), FIG. 1879 (SEQ ID NO:1879), FIG. 1880 (SEQ ID NO:1880), FIG. 1882 (SEQ ID NO:1882), FIG. 1884 (SEQ ID NO:1884), FIG. 1886A-B (SEQ ID NO:1886), FIG. 1888 (SEQ ID NO:1888), FIG. 1890 (SEQ ID NO:1890), FIG. 1892 (SEQ ID NO:1892), FIG. 1894 (SEQ ID NO:1894), FIG. 1896 (SEQ ID NO:1896), FIG. 1898A-B (SEQ ID NO:1898), FIG. 1900 (SEQ ID NO:1900), FIG. 1902 (SEQ ID NO:1902), FIG. 1904 (SEQ ID NO:1904), FIG. 1906A-B (SEQ ID NO:1906), FIG. 1907 (SEQ ID NO:1907), FIG. 1909 (SEQ ID NO:1909), FIG. 1911 (SEQ ID NO:1911), FIG. 1912 (SEQ ID NO:1912), FIG. 1914 (SEQ ID NO:1914), FIG. 1916A-B (SEQ ID NO:1916), FIG. 1918 (SEQ ID NO:1918), FIG. 1920A-B (SEQ ID NO:1920), FIG. 1921 (SEQ ID NO:1921), FIG. 1923 (SEQ ID NO:1923), FIG. 1925 (SEQ ID NO:1925), FIG. 1927 (SEQ ID NO:1927), FIG. 1929 (SEQ ID NO:1929), FIG. 1931 (SEQ ID NO:1931), FIG. 1933 (SEQ ID NO:1933), FIG. 1935 (SEQ ID NO:1935), FIG. 1937 (SEQ ID NO:1937), FIG. 1939 (SEQ ID NO:1939), FIG. 1941 (SEQ ID NO:1941), FIG. 1943 (SEQ ID NO:1943), FIG. 1945 (SEQ ID NO:1945), FIG. 1947 (SEQ ID NO:1947), FIG. 1949 (SEQ ID NO:1949), FIG. 1951 (SEQ ID NO:1951), FIG. 1953 (SEQ ID NO:1953), FIG. 1955 (SEQ ID NO:1955), FIG. 1957 (SEQ ID NO:1957), FIG. 1959 (SEQ ID NO:1959), FIG. 1961 (SEQ ID NO:1961), FIG. 1963A-B (SEQ ID NO:1963), FIG. 1965 (SEQ ID NO:1965), FIG. 1967 (SEQ ID NO:1967), FIG. 1969 (SEQ ID NO:1969), FIG. 1971 (SEQ ID NO:1971), FIG. 1973 (SEQ ID NO:1973), FIG. 1975 (SEQ ID NO:1975), FIG. 1977 (SEQ ID NO:1977), FIG. 1979 (SEQ ID NO:1979), FIG. 1981 (SEQ ID NO:1981), FIG. 1983 (SEQ ID NO:1983), FIG. 1985 (SEQ ID NO:1985), FIG. 1987 (SEQ ID NO:1987), FIG. 1989 (SEQ ID NO:1989), FIG. 1991 (SEQ ID NO:1991), FIG. 1993 (SEQ ID NO:1993), FIG. 1995 (SEQ ID NO:1995), FIG. 1997 (SEQ ID NO:1997), FIG. 1999 (SEQ ID NO:1999), FIG. 2001 (SEQ ID NO:2001), FIG. 2003 (SEQ ID NO:2003), FIG. 2005A-B (SEQ ID NO:2005), FIG. 2007 (SEQ ID NO:2007), FIG. 2009 (SEQ ID NO:2009), FIG. 2011 (SEQ ID NO:2011), FIG. 2013 (SEQ ID NO:2013), FIG. 2015 (SEQ ID NO:2015), FIG. 2017A-B (SEQ ID NO:2017), FIG. 2019A-B (SEQ ID NO:2019), FIG. 2021 (SEQ ID NO:2021), FIG. 2023 (SEQ ID NO:2023), FIG. 2025 (SEQ ID NO:2025), FIG. 2027 (SEQ ID NO:2027), FIG. 2029 (SEQ ID NO:2029), FIG. 2031 (SEQ ID NO:2031), FIG. 2033 (SEQ ID NO:2033), FIG. 2035 (SEQ ID NO:2035), FIG. 2037 (SEQ ID NO:2037), FIG. 2039 (SEQ ID NO:2039), FIG. 2041 (SEQ ID NO:2041), FIG. 2043 (SEQ ID NO:2043), FIG. 2045 (SEQ ID NO:2045), FIG. 2047 (SEQ ID NO:2047), FIG. 2049A-B (SEQ ID NO:2049), FIG. 2051 (SEQ ID NO:2051), FIG. 2053 (SEQ ID NO:2053), FIG. 2055A-B (SEQ ID NO:2055), FIG. 2057 (SEQ ID NO:2057), FIG. 2059 (SEQ ID NO:2059), FIG. 2061 (SEQ ID NO:2061), FIG. 2063 (SEQ ID NO:2063), FIG. 2065 (SEQ ID NO:2065), FIG. 2067 (SEQ ID NO:2067), FIG. 2069 (SEQ ID NO:2069), FIG. 2071A-B (SEQ ID NO:2071), FIG. 2073 (SEQ ID NO:2073), FIG. 2075 (SEQ ID NO:2075), FIG. 2077 (SEQ ID NO:2077), FIG. 2079 (SEQ ID NO:2079), FIG. 2081 (SEQ ID NO:2081), FIG. 2083 (SEQ ID NO:2083), FIG. 2085 (SEQ ID NO:2085), FIG. 2087 (SEQ ID NO:2087), FIG. 2089 (SEQ ID NO:2089), FIG. 2091 (SEQ ID NO:2091), FIG. 2093 (SEQ ID NO:2093), FIG. 2095 (SEQ ID NO:2095), FIG. 2097 (SEQ ID NO:2097), FIG. 2099 (SEQ ID NO:2099), FIG. 2101 (SEQ ID NO:2101), FIG. 2103 (SEQ ID NO:2103), FIG. 2105 (SEQ ID NO:2105), FIG. 2107 (SEQ ID NO:2107), FIG. 2109 (SEQ ID NO:2109), FIG. 2111 (SEQ ID NO:2111), FIG. 2113 (SEQ ID NO:2113), FIG. 2115 (SEQ ID NO:2115), FIG. 2117 (SEQ ID NO:2117), FIG. 2119 (SEQ ID NO:2119), FIG. 2121 (SEQ ID NO:2121), FIG. 2123 (SEQ ID NO:2123), FIG. 2125 (SEQ ID NO:2125), FIG. 2127 (SEQ ID NO:2127), FIG. 2129 (SEQ ID NO:2129), FIG. 2131 (SEQ ID NO:2131), FIG. 2133 (SEQ ID NO:2133), FIG. 2135 (SEQ ID NO:2135), FIG. 2137 (SEQ ID NO:2137), FIG. 2139 (SEQ ID NO:2139), FIG. 2141 (SEQ ID NO:2141), FIG. 2143 (SEQ ID NO:2143), FIG. 2145A-B (SEQ ID NO:2145), FIG. 2147 (SEQ ID NO:2147), FIG. 2149 (SEQ ID NO:2149), FIG. 2151 (SEQ ID NO:2151), FIG. 2153 (SEQ ID NO:2153), FIG. 2155 (SEQ ID NO:2155), FIG. 2157 (SEQ ID NO:2157), FIG. 2159 (SEQ ID NO:2159), FIG. 2161A-B (SEQ ID NO:2161), FIG. 2162 (SEQ ID NO:2162), FIG. 2164 (SEQ ID NO:2164), FIG. 2166A-B (SEQ ID NO:2166), FIG. 2168 (SEQ ID NO:2168), FIG. 2169 (SEQ ID NO:2169), FIG. 2171A-B (SEQ ID NO:2171), FIG. 2173 (SEQ ID NO:2173), FIG. 2175A-B (SEQ ID NO:2175), FIG. 2177 (SEQ ID NO:2177), FIG. 2179 (SEQ ID NO:2179), FIG. 2181 (SEQ ID NO:2181), FIG. 2183 (SEQ ID NO:2183), FIG. 2185A-B (SEQ ID NO:2185), FIG. 2187 (SEQ ID NO:2187), FIG. 2189 (SEQ ID NO:2189), FIG. 2190A-B (SEQ ID NO:2190), FIG. 2192 (SEQ ID NO:2192), FIG. 2194 (SEQ ID NO:2194), FIG. 2196 (SEQ ID NO:2196), FIG. 2198 (SEQ ID NO:2198), FIG. 2200 (SEQ ID NO:2200), FIG. 2202 (SEQ ID NO:2202), FIG. 2203 (SEQ ID NO:2203), FIG. 2205 (SEQ ID NO:2205), FIG. 2206A-B (SEQ ID NO:2206), FIG. 2208 (SEQ ID NO:2208), FIG. 2210 (SEQ ID NO:2210), FIG. 2211 (SEQ ID NO:2211), FIG. 2213 (SEQ ID NO:2213), FIG. 2215 (SEQ ID NO:2215), FIG. 2217 (SEQ ID NO:2217), FIG. 2219 (SEQ ID NO:2219), FIG. 2221 (SEQ ID NO:2221), FIG. 2223 (SEQ ID NO:2223), FIG. 2225 (SEQ ID NO:2225), FIG. 2227 (SEQ ID NO:2227), FIG. 2229 (SEQ ID NO:2229), FIG. 2231 (SEQ ID NO:2231), FIG. 2233 (SEQ ID NO:2233), FIG. 2235 (SEQ ID NO:2235), FIG. 2237 (SEQ ID NO:2237), FIG. 2239 (SEQ ID NO:2239), FIG. 2241 (SEQ ID NO:2241), FIG. 2243 (SEQ ID NO:2243), FIG. 2245 (SEQ ID NO:2245), FIG. 2247 (SEQ ID NO:2247), FIG. 2249 (SEQ ID NO:2249), FIG. 2251 (SEQ ID NO:2251), FIG. 2253 (SEQ ID NO:2253), FIG. 2255 (SEQ ID NO:2255), FIG. 2257 (SEQ ID NO:2257), FIG. 2259 (SEQ ID NO:2259), FIG. 2261 (SEQ ID NO:2261), FIG. 2263 (SEQ ID NO:2263), FIG. 2265 (SEQ ID NO:2265), FIG. 2267 (SEQ ID NO:2267), FIG. 2269 (SEQ ID NO:2269), FIG. 2271 (SEQ ID NO:2271), FIG. 2273 (SEQ ID NO:2273), FIG. 2275 (SEQ ID NO:2275), FIG. 2277 (SEQ ID NO:2277), FIG. 2279 (SEQ ID NO:2279), FIG. 2281 (SEQ ID NO:2281), FIG. 2283 (SEQ ID NO:2283), FIG. 2285 (SEQ ID NO:2285), FIG. 2287 (SEQ ID NO:2287), FIG. 2289 (SEQ ID NO:2289), FIG. 2291 (SEQ ID NO:2291), FIG. 2293 (SEQ ID NO:2293), FIG. 2295 (SEQ ID NO:2295), FIG. 2297 (SEQ ID NO:2297), FIG. 2299 (SEQ ID NO:2299), FIG. 2300 (SEQ ID NO:2300), FIG. 2302A-B (SEQ ID NO:2302), FIG. 2304 (SEQ ID NO:2304), FIG. 2306 (SEQ ID NO:2306), FIG. 2308 (SEQ ID NO:2308), FIG. 2309 (SEQ ID NO:2309), FIG. 2311 (SEQ ID NO:2311), FIG. 2313 (SEQ ID NO:2313), FIG. 2315 (SEQ ID NO:2315), FIG. 2317 (SEQ ID NO:2317), FIG. 2318 (SEQ ID NO:2318), FIG. 2320A-B (SEQ ID NO:2320), FIG. 2322 (SEQ ID NO:2322), FIG. 2324 (SEQ ID NO:2324), FIG. 2326 (SEQ ID NO:2326), FIG. 2328 (SEQ ID NO:2328), FIG. 2330A-B (SEQ ID NO:2330), FIG. 2332 (SEQ ID NO:2332), FIG. 2334 (SEQ ID NO:2334), FIG. 2336A-B (SEQ ID NO:2336), FIG. 2338 (SEQ ID NO:2338), FIG. 2340 (SEQ ID NO:2340), FIG. 2342 (SEQ ID NO:2342), FIG. 2344 (SEQ ID NO:2344), FIG. 2346 (SEQ ID NO:2346), FIG. 2348 (SEQ ID NO:2348), FIG. 2350 (SEQ ID NO:2350), FIG. 2352 (SEQ ID NO:2352), FIG. 2354A-B (SEQ ID NO:2354), FIG. 2356 (SEQ ID NO:2356), FIG. 2358 (SEQ ID NO:2358), FIG. 2360 (SEQ ID NO:2360), FIG. 2362 (SEQ ID NO:2362), FIG. 2364 (SEQ ID NO:2364), FIG. 2366 (SEQ ID NO:2366), FIG. 2368 (SEQ ID NO:2368), FIG. 2370 (SEQ ID NO:2370), FIG. 2372 (SEQ ID NO:2372), FIG. 2374 (SEQ ID NO:2374), FIG. 2376A-B (SEQ ID NO:2376), FIG. 2377 (SEQ ID NO:2377), FIG. 2379 (SEQ ID NO:2379), FIG. 2381 (SEQ ID NO:2381), FIG. 2382 (SEQ ID NO:2382), FIG. 2384 (SEQ ID NO:2384), FIG. 2385 (SEQ ID NO:2385), FIG. 2387 (SEQ ID NO:2387), FIG. 2389 (SEQ ID NO:2389), FIG. 2390 (SEQ ID NO:2390), FIG. 2392 (SEQ ID NO:2392), FIG. 2394 (SEQ ID NO:2394), FIG. 2395A-B (SEQ ID NO:2395), FIG. 2397 (SEQ ID NO:2397), FIG. 2399 (SEQ ID NO:2399), FIG. 2400 (SEQ ID NO:2400), FIG. 2401 (SEQ ID NO:2401), FIG. 2403 (SEQ ID NO:2403), FIG. 2405 (SEQ ID NO:2405), FIG. 2406 (SEQ ID NO:2406), FIG. 2407 (SEQ ID NO:2407), FIG. 2409 (SEQ ID NO:2409), FIG. 2411 (SEQ ID NO:2411), FIG. 2413 (SEQ ID NO:2413), FIG. 2414 (SEQ ID NO:2414), FIG. 2416 (SEQ ID NO:2416), FIG. 2417 (SEQ ID NO:2417), FIG. 2419 (SEQ ID NO:2419), FIG. 2421 (SEQ ID NO:2421), FIG. 2423 (SEQ ID NO:2423), FIG. 2425 (SEQ ID NO:2425), FIG. 2427 (SEQ ID NO:2427), FIG. 2428 (SEQ ID NO:2428), FIG. 2429 (SEQ ID NO:2429), FIG. 2431 (SEQ ID NO:2431), FIG. 2433 (SEQ ID NO:2433), FIG. 2435 (SEQ ID NO:2435), FIG. 2437 (SEQ ID NO:2437), FIG. 2439 (SEQ ID NO:2439) or FIG. 2441 (SEQ ID NO:2441).

3. Isolated nucleic acid having at least 80% nucleic acid sequence identity to a nucleotide sequence selected from the group consisting of the full-length coding sequence of the nucleotide sequence shown in FIG. 1 (SEQ ID NO:1), FIG. 3 (SEQ ID NO:3), FIG. 5 (SEQ ID NO:5), FIG. 7 (SEQ ID NO:7), FIG. 9 (SEQ ID NO:9), FIG. 11 (SEQ ID NO:11), FIG. 13 (SEQ ID NO:13), FIG. 15 (SEQ ID NO:15), FIG. 17 (SEQ ID NO:17), FIG. 19 (SEQ ID NO:19), FIG. 21 (SEQ ID NO:21), FIG. 23 (SEQ ID NO:23), FIG. 25 (SEQ ID NO:25), FIG. 27 (SEQ ID NO:27), FIG. 29 (SEQ ID NO:29), FIG. 31 (SEQ ID NO:31), FIG. 33 (SEQ ID NO:33), FIG. 35 (SEQ ID NO:35), FIG. 37 (SEQ ID NO:37), FIG. 39 (SEQ ID NO:39), FIG. 41 (SEQ ID NO:41), FIG. 43 (SEQ ID NO:43), FIG. 45 (SEQ ID NO:45), FIG. 47 (SEQ ID NO:47), FIG. 49 (SEQ ID NO:49), FIG. 51 (SEQ ID NO:51), FIG. 53 (SEQ ID NO:53), FIG. 55 (SEQ ID NO:55), FIG. 57 (SEQ ID NO:57), FIG. 59 (SEQ ID NO:59), FIG. 61 (SEQ ID NO:61), FIG. 63 (SEQ ID NO:63), FIG. 65 (SEQ ID NO:65), FIG. 67 (SEQ ID NO:67), FIG. 69 (SEQ ID NO:69), FIG. 71 (SEQ ID NO:71), FIG. 73 (SEQ ID NO:73), FIG. 75 (SEQ ID NO:75), FIG. 77 (SEQ ID NO:77), FIG. 79 (SEQ ID NO:79), FIG. 81 (SEQ ID NO:81), FIG. 83 (SEQ ID NO:83), FIG. 85 (SEQ ID NO:85), FIG. 87 (SEQ ID NO:87), FIG. 89 (SEQ ID NO:89), FIG. 91 (SEQ ID NO:91), FIG. 93 (SEQ ID NO:93), FIG. 95 (SEQ ID NO:95), FIG. 97 (SEQ ID NO:97), FIG. 99 (SEQ ID NO:99), FIG. 101 (SEQ ID NO:101), FIG. 103 (SEQ ID NO:103), FIG. 105 (SEQ ID NO:105), FIG. 107 (SEQ ID NO:107), FIG. 109 (SEQ ID NO:109), FIG. 111 (SEQ ID NO:111), FIG. 113 (SEQ ID NO:113), FIG. 115 (SEQ ID NO:115), FIG. 117 (SEQ ID NO:117), FIG. 119 (SEQ ID NO:119), FIG. 120 (SEQ ID NO:120), FIG. 122 (SEQ ID NO:122), FIG. 124 (SEQ ID NO:124), FIG. 126 (SEQ ID NO:126), FIG. 128 (SEQ ID NO:128), FIG. 130 (SEQ ID NO:130), FIG. 132 (SEQ ID NO:132), FIG. 134 (SEQ ID NO:134), FIG. 136 (SEQ ID NO:136), FIG. 138 (SEQ ID NO:138), FIG. 140 (SEQ ID NO:140), FIG. 142 (SEQ ID NO:142), FIG. 144 (SEQ ID NO:144), FIG. 146 (SEQ ID NO:146), FIG. 148 (SEQ ID NO:148), FIG. 150 (SEQ ID NO:150), FIG. 152 (SEQ ID NO:152), FIG. 154 (SEQ ID NO:154), FIG. 156 (SEQ ID NO:156), FIG. 158 (SEQ ID NO:158), FIG. 160 (SEQ ID NO:160), FIG. 162 (SEQ ID NO:162), FIG. 164 (SEQ ID NO:164), FIG. 166 (SEQ ID NO:166), FIG. 168 (SEQ ID NO:168), FIG. 170 (SEQ ID NO:170), FIG. 172 (SEQ ID NO:172), FIG. 174 (SEQ ID NO:174), FIG. 176 (SEQ ID NO:176), FIG. 178 (SEQ ID NO:178), FIG. 180 (SEQ ID NO:180), FIG. 182 (SEQ ID NO:182), FIG. 184 (SEQ ID NO:184), FIG. 186 (SEQ ID NO:186), FIG. 188 (SEQ ID NO:188), FIG. 190 (SEQ ID NO:190), FIG. 192 (SEQ ID NO:192), FIG. 194 (SEQ ID NO:194), FIG. 196 (SEQ ID NO:196), FIG. 198 (SEQ ID NO:198), FIG. 200 (SEQ ID NO:200), FIG. 202 (SEQ ID NO:202), FIG. 204 (SEQ ID NO:204), FIG. 206 (SEQ ID NO:206), FIG. 208 (SEQ ID NO:208), FIG. 210 (SEQ ID NO:210), FIG. 212 (SEQ ID NO:212), FIG. 214 (SEQ ID NO:214), FIG. 216 (SEQ ID NO:216), FIG. 218 (SEQ ID NO:218), FIG. 220 (SEQ ID NO:220), FIG. 222 (SEQ ID NO:222), FIG. 224 (SEQ ID NO:224), FIG. 226 (SEQ ID NO:226), FIG. 228 (SEQ ID NO:228), FIG. 230 (SEQ ID NO:231), FIG. 232 (SEQ ID NO:232), FIG. 234 (SEQ ID NO:234), FIG. 236 (SEQ ID NO:236), FIG. 238 (SEQ ID NO:238), FIG. 240 (SEQ ID NO:240), FIG. 242 (SEQ ID NO:242), FIG. 244 (SEQ ID NO:244), FIG. 246 (SEQ ID NO:246), FIG. 248 (SEQ ID NO:248), FIG. 250 (SEQ ID NO:250), FIG. 252 (SEQ ID NO:252), FIG. 253 (SEQ ID NO:253), FIG. 255 (SEQ ID NO:255), FIG. 257 (SEQ ID NO:257), FIG. 259 (SEQ ID NO:259), FIG. 261 (SEQ ID NO:261), FIG. 263 (SEQ ID NO:263), FIG. 265 (SEQ ID NO:265), FIG. 267 (SEQ ID NO:267), FIG. 269 (SEQ ID NO:269), FIG. 271 (SEQ ID NO:271), FIG. 273 (SEQ ID NO:273), FIG. 275 (SEQ ID NO:275), FIG. 277 (SEQ ID NO:277), FIG. 279 (SEQ ID NO:279), FIG. 281 (SEQ ID NO:281), FIG. 283 (SEQ ID NO:283), FIG. 285 (SEQ ID NO:285), FIG. 287 (SEQ ID NO:287), FIG. 289 (SEQ ID NO:289), FIG. 291 (SEQ ID NO:291), FIG. 293 (SEQ ID NO:293), FIG. 295 (SEQ ID NO:295), FIG. 297 (SEQ ID NO:297), FIG. 299 (SEQ ID NO:299), FIG. 301 (SEQ ID NO:301), FIG. 303 (SEQ ID NO:303), FIG. 305 (SEQ ID NO:305), FIG. 307 (SEQ ID NO:307), FIG. 309 (SEQ ID NO:309), FIG. 311 (SEQ ID NO:311), FIG. 313 (SEQ ID NO:313), FIG. 315 (SEQ ID NO:315), FIG. 317 (SEQ ID NO:317), FIG. 319 (SEQ ID NO:319), FIG. 321 (SEQ ID NO:321), FIG. 323 (SEQ ID NO:323), FIG. 325 (SEQ ID NO:325), FIG. 327 (SEQ ID NO:327), FIG. 329 (SEQ ID NO:329), FIG. 331 (SEQ ID NO:331), FIG. 333 (SEQ ID NO:333), FIG. 335 (SEQ ID NO:335), FIG. 337 (SEQ ID NO:337), FIG. 339 (SEQ ID NO:339), FIG. 341 (SEQ ID NO:341), FIG. 343 (SEQ ID NO:343), FIG. 345 (SEQ ID NO:345), FIG. 347 (SEQ ID NO:347), FIG. 349 (SEQ ID NO:349), FIG. 351 (SEQ ID NO:351), FIG. 353 (SEQ ID NO:353), FIG. 355 (SEQ ID NO:355), FIG. 357 (SEQ ID NO:357), FIG. 359 (SEQ ID NO:359), FIG. 361 (SEQ ID NO:361), FIG. 363 (SEQ ID NO:363), FIG. 365 (SEQ ID NO:365), FIG. 367 (SEQ ID NO:367), FIG. 369 (SEQ ID NO:369), FIG. 371 (SEQ ID NO:371), FIG. 373 (SEQ ID NO:373), FIG. 375 (SEQ ID NO:375), FIG. 377 (SEQ ID NO:377), FIG. 379 (SEQ ID NO:379), FIG. 381 (SEQ ID NO:381), FIG. 383 (SEQ ID NO:383), FIG. 385 (SEQ ID NO:385), FIG. 387 (SEQ ID NO:387), FIG. 389 (SEQ ID NO:389), FIG. 391 (SEQ ID NO:391), FIG. 393 (SEQ ID NO:393), FIG. 395 (SEQ ID NO:395), FIG. 397 (SEQ ID NO:397), FIG. 399 (SEQ ID NO:399), FIG. 401 (SEQ ID NO:401), FIG. 403 (SEQ ID NO:403), FIG. 405 (SEQ ID NO:405), FIG. 407 (SEQ ID NO:407), FIG. 409 (SEQ ID NO:409), FIG. 411 (SEQ ID NO:411), FIG. 413 (SEQ ID NO:413), FIG. 415 (SEQ ID NO:415), FIG. 417 (SEQ ID NO:417), FIG. 419 (SEQ ID NO:419), FIG. 421 (SEQ ID NO:421), FIG. 423 (SEQ ID NO:423), FIG. 425 (SEQ ID NO:425), FIG. 427 (SEQ ID NO:427), FIG. 429 (SEQ ID NO:429), FIG. 431 (SEQ ID NO:431), FIG. 433 (SEQ ID NO:433), FIG. 435 (SEQ ID NO:435), FIG. 437 (SEQ ID NO:437), FIG. 439 (SEQ ID NO:439), FIG. 441 (SEQ ID NO:441), FIG. 443 (SEQ ID NO:443), FIG. 445 (SEQ ID NO:445), FIG. 447 (SEQ ID NO:447), FIG. 449 (SEQ ID NO:449), FIG. 451 (SEQ ID NO:451), FIG. 453 (SEQ ID NO:453), FIG. 455 (SEQ ID NO:455), FIG. 457 (SEQ ID NO:457), FIG. 459 (SEQ ID NO:459), FIG. 461 (SEQ ID NO:461), FIG. 463 (SEQ ID NO:463), FIG. 465 (SEQ ID NO:465), FIG. 467 (SEQ ID NO:467), FIG. 469 (SEQ ID NO:469), FIG. 471 (SEQ ID NO:471), FIG. 473 (SEQ ID NO:473), FIG. 475 (SEQ ID NO:475), FIG. 477 (SEQ ID NO:477), FIG. 479 (SEQ ID NO:479), FIG. 481 (SEQ ID NO:481), FIG. 483 (SEQ ID NO:483), FIG. 485 (SEQ ID NO:485), FIG. 487 (SEQ ID NO:487), FIG. 489 (SEQ ID NO:489), FIG. 491 (SEQ ID NO:491), FIG. 493 (SEQ ID NO:493), FIG. 495 (SEQ ID NO:495), FIG. 497 (SEQ ID NO:497), FIG. 499 (SEQ ID NO:499), FIG. 501 (SEQ ID NO:501), FIG. 503 (SEQ ID NO:503), FIG. 505 (SEQ ID NO:505), FIG. 507 (SEQ ID NO:507), FIG. 509 (SEQ ID NO:509), FIG. 511 (SEQ ID NO:511), FIG. 513 (SEQ ID NO:513), FIG. 515 (SEQ ID NO:515), FIG. 517 (SEQ ID NO:517), FIG. 519 (SEQ ID NO:519), FIG. 521 (SEQ ID NO:521), FIG. 523 (SEQ ID NO:523), FIG. 525 (SEQ ID NO:525), FIG. 527 (SEQ ID NO:527), FIG. 529 (SEQ ID NO:529), FIG. 531 (SEQ ID NO:531), FIG. 533 (SEQ ID NO:533), FIG. 535 (SEQ ID NO:535), FIG. 537 (SEQ ID NO:537), FIG. 539 (SEQ ID NO:539), FIG. 541 (SEQ ID NO:541), FIG. 543 (SEQ ID NO:543), FIG. 545 (SEQ ID NO:545), FIG. 547 (SEQ ID NO:547), FIG. 548 (SEQ ID NO:548), FIG. 550 (SEQ ID NO:550), FIG. 552 (SEQ ID NO:552), FIG. 554 (SEQ ID NO:554), FIG. 556 (SEQ ID NO:556), FIG. 558 (SEQ ID NO:558), FIG. 560 (SEQ ID NO:560), FIG. 562 (SEQ ID NO:562), FIG. 564 (SEQ ID NO:564), FIG. 566 (SEQ ID NO:566), FIG. 568 (SEQ ID NO:568), FIG. 570 (SEQ ID NO:570), FIG. 572 (SEQ ID NO:572), FIG. 574 (SEQ ID NO:574), FIG. 576 (SEQ ID NO:576), FIG. 578 (SEQ ID NO:578), FIG. 580 (SEQ ID NO:580), FIG. 582 (SEQ ID NO:582), FIG. 584 (SEQ ID NO:584), FIG. 586 (SEQ ID NO:586), FIG. 588 (SEQ ID NO:588), FIG. 590 (SEQ ID NO:590), FIG. 592 (SEQ ID NO:592), FIG. 594 (SEQ ID NO:594), FIG. 596 (SEQ ID NO:596), FIG. 598 (SEQ ID NO:598), FIG. 600 (SEQ ID NO:600), FIG. 602 (SEQ ID NO:602), FIG. 604 (SEQ ID NO:604), FIG. 606 (SEQ ID NO:606), FIG. 608 (SEQ ID NO:608), FIG. 610 (SEQ ID NO:610), FIG. 612 (SEQ ID NO:612), FIG. 614 (SEQ ID NO:614), FIG. 616 (SEQ ID NO:616), FIG. 618 (SEQ ID NO:618), FIG. 620 (SEQ ID NO:620), FIG. 622 (SEQ ID NO:622), FIG. 624 (SEQ ID NO:624), FIG. 626 (SEQ ID NO:626), FIG. 628 (SEQ ID NO:628), FIG. 630 (SEQ ID NO:630), FIG. 632 (SEQ ID NO:632), FIG. 634 (SEQ ID NO:634), FIG. 636 (SEQ ID NO:636), FIG. 638 (SEQ ID NO:638), FIG. 640 (SEQ ID NO:640), FIG. 642 (SEQ ID NO:642), FIG. 644 (SEQ ID NO:644), FIG. 646 (SEQ ID NO:646), FIG. 648 (SEQ ID NO:648), FIG. 650 (SEQ ID NO:650), FIG. 652 (SEQ ID NO:652), FIG. 654 (SEQ ID NO:654), FIG. 656 (SEQ ID NO:656), FIG. 658 (SEQ ID NO:658), FIG. 660 (SEQ ID NO:660), FIG. 662 (SEQ ID NO:662), FIG. 664 (SEQ ID NO:664), FIG. 666 (SEQ ID NO:666), FIG. 668 (SEQ ID NO:668), FIG. 670 (SEQ ID NO:670), FIG. 672 (SEQ ID NO:672), FIG. 674 (SEQ ID NO:674), FIG. 676 (SEQ ID NO:676), FIG. 678 (SEQ ID NO:678), FIG. 680 (SEQ ID NO:680), FIG. 682 (SEQ ID NO:682), FIG. 684 (SEQ ID NO:684), FIG. 686 (SEQ ID NO:686), FIG. 688 (SEQ ID NO:688), FIG. 690 (SEQ ID NO:690), FIG. 692 (SEQ ID NO:692), FIG. 694 (SEQ ID NO:694), FIG. 696 (SEQ ID NO:696), FIG. 698 (SEQ ID NO:698), FIG. 700 (SEQ ID NO:700), FIG. 702 (SEQ ID NO:702), FIG. 704 (SEQ ID NO:704), FIG. 706 (SEQ ID NO:706), FIG. 708 (SEQ ID NO:708), FIG. 710 (SEQ ID NO:710), FIG. 712 (SEQ ID NO:712), FIG. 714 (SEQ ID NO:714), FIG. 716 (SEQ ID NO:716), FIG. 718 (SEQ ID NO:718), FIG. 720 (SEQ ID NO:720), FIG. 722 (SEQ ID NO:722), FIG. 724 (SEQ ID NO:724), FIG. 726 (SEQ ID NO:726), FIG. 728 (SEQ ID NO:728), FIG. 730 (SEQ ID NO:730), FIG. 732 (SEQ ID NO:732), FIG. 734 (SEQ ID NO:734), FIG. 736 (SEQ ID NO:736), FIG. 738 (SEQ ID NO:738), FIG. 740 (SEQ ID NO:740), FIG. 742 (SEQ ID NO:742), FIG. 744 (SEQ ID NO:744), FIG. 746 (SEQ ID NO:746), FIG. 748 (SEQ ID NO:748), FIG. 750 (SEQ ID NO:750), FIG. 752 (SEQ ID NO:752), FIG. 754 (SEQ ID NO:754), FIG. 756 (SEQ ID NO:756), FIG. 758 (SEQ ID NO:758), FIG. 760 (SEQ ID NO:760), FIG. 762 (SEQ ID NO:762), FIG. 764 (SEQ ID NO:764), FIG. 766 (SEQ ID NO:766), FIG. 768 (SEQ ID NO:768), FIG. 770 (SEQ ID NO:770), FIG. 772 (SEQ ID NO:772), FIG. 774 (SEQ ID NO:774), FIG. 776 (SEQ ID NO:776), FIG. 778 (SEQ ID NO:778), FIG. 780 (SEQ ID NO:780), FIG. 782 (SEQ ID NO:782), FIG. 784 (SEQ ID NO:784), FIG. 786 (SEQ ID NO:786), FIG. 788 (SEQ ID NO:788), FIG. 790 (SEQ ID NO:790), FIG. 792 (SEQ ID NO:792), FIG. 794 (SEQ ID NO:794), FIG. 796 (SEQ ID NO:796), FIG. 798 (SEQ ID NO:798), FIG. 800 (SEQ ID NO:800), FIG. 802 (SEQ ID NO:802), FIG. 804 (SEQ ID NO:804), FIG. 806 (SEQ ID NO:806), FIG. 808 (SEQ ID NO:808), FIG. 810A-B (SEQ ID NO:810), FIG. 812 (SEQ ID NO:812), FIG. 814 (SEQ ID NO:814), FIG. 816 (SEQ ID NO:816), FIG. 818 (SEQ ID NO:818), FIG. 820 (SEQ ID NO:820), FIG. 822 (SEQ ID NO:822), FIG. 824 (SEQ ID NO:824), FIG. 826 (SEQ ID NO:826), FIG. 828 (SEQ ID NO:828), FIG. 830 (SEQ ID NO:830), FIG. 832 (SEQ ID NO:832), FIG. 834 (SEQ ID NO:834), FIG. 836 (SEQ ID NO:836), FIG. 838 (SEQ ID NO:838), FIG. 840 (SEQ ID NO:840), FIG. 842 (SEQ ID NO:842), FIG. 844 (SEQ ID NO:844), FIG. 846 (SEQ ID NO:846), FIG. 848 (SEQ ID NO:848), FIG. 850 (SEQ ID NO:850), FIG. 852 (SEQ ID NO:852), FIG. 854 (SEQ ID NO:854), FIG. 856 (SEQ ID NO:856), FIG. 857A-B (SEQ ID NO:857), FIG. 859A-B (SEQ ID NO:859), FIG. 861 (SEQ ID NO:861), FIG. 863 (SEQ ID NO:863), FIG. 865 (SEQ ID NO:865), FIG. 867 (SEQ ID NO:867), FIG. 869 (SEQ ID NO:869), FIG. 871 (SEQ ID NO:871), FIG. 873 (SEQ ID NO:873), FIG. 875 (SEQ ID NO:875), FIG. 877 (SEQ ID NO:877), FIG. 879 (SEQ ID NO:879), FIG. 881A-B (SEQ ID NO:881), FIG. 883 (SEQ ID NO:883), FIG. 885 (SEQ ID NO:885), FIG. 887 (SEQ ID NO:887), FIG. 889 (SEQ ID NO:889), FIG. 891 (SEQ ID NO:891), FIG. 893 (SEQ ID NO:893), FIG. 895 (SEQ ID NO:895), FIG. 897 (SEQ ID NO:897), FIG. 899 (SEQ ID NO:899), FIG. 901 (SEQ ID NO:901), FIG. 903 (SEQ ID NO:903), FIG. 905A-C (SEQ ID NO:905), FIG. 907 (SEQ ID NO:907), FIG. 909 (SEQ ID NO:909), FIG. 911 (SEQ ID NO:911), FIG. 913 (SEQ ID NO:913), FIG. 915 (SEQ ID NO:915), FIG. 917 (SEQ ID NO:917), FIG. 919A-B (SEQ ID NO:919), FIG. 921 (SEQ ID NO:921), FIG. 923 (SEQ ID NO:923), FIG. 925 (SEQ ID NO:925), FIG. 927 (SEQ ID NO:927), FIG. 929 (SEQ ID NO:929), FIG. 931 (SEQ ID NO:931), FIG. 933A-B (SEQ ID NO:933), FIG. 935 (SEQ ID NO:935), FIG. 937A-B (SEQ ID NO:937), FIG. 939A-B (SEQ ID NO:939), FIG. 941A-B (SEQ ID NO:941), FIG. 942A-B (SEQ ID NO:942), FIG. 944 (SEQ ID NO:944), FIG. 946 (SEQ ID NO:946), FIG. 948A-C (SEQ ID NO:948), FIG. 950 (SEQ ID NO:950), FIG. 952 (SEQ ID NO:952), FIG. 954A-B (SEQ ID NO:954), FIG. 956A-B (SEQ ID NO:956), FIG. 957A-B (SEQ ID NO:957), FIG. 959 (SEQ ID NO:959), FIG. 961 (SEQ ID NO:961), FIG. 962 (SEQ ID NO:962), FIG. 964 (SEQ ID NO:964), FIG. 966 (SEQ ID NO:966), FIG. 967 (SEQ ID NO:967), FIG. 969A-B (SEQ ID NO:969), FIG. 971A-B (SEQ ID NO:971), FIG. 973 (SEQ ID NO:973), FIG. 975 (SEQ ID NO:975), FIG. 977A-B (SEQ ID NO:977), FIG. 979 (SEQ ID NO:979), FIG. 981 (SEQ ID NO:981), FIG. 982 (SEQ ID NO:982), FIG. 984 (SEQ ID NO:984), FIG. 986 (SEQ ID NO:986), FIG. 988 (SEQ ID NO:988), FIG. 989A-D (SEQ ID NO:989), FIG. 991 (SEQ ID NO:991), FIG. 993A-C (SEQ ID NO:993), FIG. 995 (SEQ ID NO:995), FIG. 997 (SEQ ID NO:997), FIG. 999A-B (SEQ ID NO:999), FIG. 1001 (SEQ ID NO:1001), FIG. 1003A-B (SEQ ID NO:1003), FIG. 1004A-B (SEQ ID NO:1004), FIG. 1005A-B (SEQ ID NO:1005), FIG. 1006A-B (SEQ ID NO:1006), FIG. 1008 (SEQ ID NO:1008), FIG. 1010A-B (SEQ ID NO:1010), FIG. 1012 (SEQ ID NO:1012), FIG. 1014 (SEQ ID NO:1014), FIG. 1016A-B (SEQ ID NO:1016), FIG. 1018 (SEQ ID NO:1018), FIG. 1020A-B (SEQ ID NO:1020), FIG. 1022 (SEQ ID NO:1022), FIG. 1024A-C (SEQ ID NO:1024), FIG. 1026A-B (SEQ ID NO:1026), FIG. 1027 (SEQ ID NO:1027), FIG. 1028A-B (SEQ ID NO:1028), FIG. 1029 (SEQ ID NO:1029), FIG. 1031 (SEQ ID NO:1031), FIG. 1032 (SEQ ID NO:1032), FIG. 1034A-C (SEQ ID NO:1034), FIG. 1036A-B (SEQ ID NO:1036), FIG. A-B 1038 (SEQ ID NO:1038), FIG. 1040 (SEQ ID NO:1040), FIG. 1042A-B (SEQ ID NO:1042), FIG. 1044A-B (SEQ ID NO:1044), FIG. 1045 (SEQ ID NO:1045), FIG. 1047 (SEQ ID NO:1047), FIG. 1049 (SEQ ID NO:1049), FIG. 1051 (SEQ ID NO:1051), FIG. 1052A-B (SEQ ID NO:1052), FIG. 1054 (SEQ ID NO:1054), FIG. 1056 (SEQ ID NO:1056), FIG. 1058 (SEQ ID NO:1058), FIG. 1059 (SEQ ID NO:1059), FIG. 1061 (SEQ ID NO:1061), FIG. 1063 (SEQ ID NO:1063), FIG. 1065 (SEQ ID NO:1065), FIG. 1067 (SEQ ID NO:1067), FIG. 1069A-B (SEQ ID NO:1069), FIG. 1071 (SEQ ID NO:1071), FIG. 1073A-B (SEQ ID NO:1073), FIG. 1075 (SEQ ID NO:1075), FIG. 1077 (SEQ ID NO:1077), FIG. 1079 (SEQ ID NO:1079), FIG. 1081 (SEQ ID NO:1081), FIG. 1083A-B (SEQ ID NO:1083), FIG. 1085 (SEQ ID NO:1085), FIG. 1087 (SEQ ID NO:1087), FIG. 1089 (SEQ ID NO:1089), FIG. 1091 (SEQ ID NO:1091), FIG. 1093A-B (SEQ ID NO:1093), FIG. 1095 (SEQ ID NO:1095), FIG. 1097 (SEQ ID NO:1097), FIG. 1099 (SEQ ID NO:1099), FIG. 1100 (SEQ ID NO:1100), FIG. 1102A-B (SEQ ID NO:1102), FIG. 1104A-B (SEQ ID NO:1104), FIG. 1106 (SEQ ID NO:1106), FIG. 1108 (SEQ ID NO:1108), FIG. 1110 (SEQ ID NO:1110), FIG. 1112 (SEQ ID NO:1112), FIG. 1114A-B (SEQ ID NO:1114), FIG. 1115 (SEQ ID NO:1115), FIG. 1117A-B (SEQ ID NO:1117), FIG. 1119 (SEQ ID NO:1119), FIG. 1121 (SEQ ID NO:1121), FIG. 1122 (SEQ ID NO:1122), FIG. 1124 (SEQ ID NO:1124), FIG. 1126 (SEQ ID NO:1126), FIG. 1127 (SEQ ID NO:1127), FIG. 1129 (SEQ ID NO:1129), FIG. 1131 (SEQ ID NO:1131), FIG. 1133 (SEQ ID NO:1133), FIG. 1135 (SEQ ID NO:1135), FIG. 1137A-B (SEQ ID NO:1137), FIG. 1139 (SEQ ID NO:1139), FIG. 1140 (SEQ ID NO:1140), FIG. 1141 (SEQ ID NO:1141), FIG. 1143A-C (SEQ ID NO:1143), FIG. 1145A-B (SEQ ID NO:1145), FIG. 1147 (SEQ ID NO:1147), FIG. 1149 (SEQ ID NO:1149), FIG. 1151 (SEQ ID NO:1151), FIG. 1153 (SEQ ID NO:1153), FIG. 1155A-B (SEQ ID NO:1155), FIG. 1157 (SEQ ID NO:1157), FIG. 1159 (SEQ ID NO:1159), FIG. 1161 (SEQ ID NO:1161), FIG. 1163 (SEQ ID NO:1163), FIG. 1165 (SEQ ID NO:1165), FIG. 1167 (SEQ ID NO:1167), FIG. 1169 (SEQ ID NO:1169), FIG. 1171 (SEQ ID NO:1171), FIG. 1173 (SEQ ID NO:1173), FIG. 1175 (SEQ ID NO:1175), FIG. 1177 (SEQ ID NO:1177), FIG. 1179 (SEQ ID NO:1179), FIG. 1181 (SEQ ID NO:1181), FIG. 1183 (SEQ ID NO:1183), FIG. 1185 (SEQ ID NO:1185), FIG. 1187 (SEQ ID NO:), FIG. 1189 (SEQ ID NO:1189), FIG. 1191 (SEQ ID NO:1191), FIG. 1193 (SEQ ID NO:1193), FIG. 1195 (SEQ ID NO:1195), FIG. 1197 (SEQ ID NO:1197), FIG. 1199 (SEQ ID NO:1199), FIG. 1201 (SEQ ID NO:1201), FIG. 1203 (SEQ ID NO:1203), FIG. 1205 (SEQ ID NO:1205), FIG. 1207 (SEQ ID NO:1207), FIG. 1209 (SEQ ID NO:1209), FIG. 1211 (SEQ ID NO:1211), FIG. 1213 (SEQ ID NO:1213), FIG. 1215 (SEQ ID NO:1215), FIG. 1217 (SEQ ID NO:1217), FIG. 1219 (SEQ ID NO:1219), FIG. 1221 (SEQ ID NO:1221), FIG. 1223A-B (SEQ ID NO:1223), FIG. 1225 (SEQ ID NO:1225), FIG. 1227 (SEQ ID NO:1227), FIG. 1229 (SEQ ID NO:1229), FIG. 1231 (SEQ ID NO:1231), FIG. 1233 (SEQ ID NO:1233), FIG. 1235 (SEQ ID NO:1235), FIG. 1237 (SEQ ID NO:1237), FIG. 1239 (SEQ ID NO:1239), FIG. 1241 (SEQ ID NO:1241), FIG. 1243 (SEQ ID NO:1243), FIG. 1245 (SEQ ID NO:1245), FIG. 1247 (SEQ ID NO:1247), FIG. 1249 (SEQ ID NO:1249), FIG. 1251 (SEQ ID NO:1251), FIG. 1253 (SEQ ID NO:1253), FIG. 1255 (SEQ ID NO:1255), FIG. 1257 (SEQ ID NO:1257), FIG. 1259 (SEQ ID NO:1259), FIG. 1261 (SEQ ID NO:1261), FIG. 1263 (SEQ ID NO:1263), FIG. 1265 (SEQ ID NO:1265), FIG. 1267 (SEQ ID NO:1267), FIG. 1269 (SEQ ID NO:1269), FIG. 1271 (SEQ ID NO:1271), FIG. 1273 (SEQ ID NO:1273), FIG. 1275 (SEQ ID NO:1275), FIG. 1277A-C (SEQ ID NO:1277), FIG. 1279 (SEQ ID NO:1279), FIG. 1281 (SEQ ID NO:1281), FIG. 1283A-B (SEQ ID NO:1283), FIG. 1285 (SEQ ID NO:1285), FIG. 1287A-B (SEQ ID NO:1287), FIG. 1289 (SEQ ID NO:1289), FIG. 1291 (SEQ ID NO:1291), FIG. 1293 (SEQ ID NO:1293), FIG. 1295 (SEQ ID NO:1295), FIG. 1297A-B (SEQ ID NO:1297), FIG. 1299 (SEQ ID NO:1299), FIG. 1301A-B (SEQ ID NO:1301), FIG. 1303 (SEQ ID NO:1303), FIG. 1305 (SEQ ID NO:1305), FIG. 1307 (SEQ ID NO:1307), FIG. 1309 (SEQ ID NO:1309), FIG. 1311 (SEQ ID NO:1311), FIG. 1313 (SEQ ID NO:1313), FIG. 1315 (SEQ ID NO:1315), FIG. 1317 (SEQ ID NO:1317), FIG. 1319 (SEQ ID NO:1319), FIG. 1321 A-B (SEQ ID NO:1321), FIG. 1323 (SEQ ID NO:1323), FIG. 1325 (SEQ ID NO:1325), FIG. 1327 (SEQ ID NO:1327), FIG. 1329 (SEQ ID NO:1329), FIG. 1331 (SEQ ID NO:1331), FIG. 1333 (SEQ ID NO:1333), FIG. 1335 (SEQ ID NO:1335), FIG. 1337 (SEQ ID NO:1337), FIG. 1339 (SEQ ID NO:1339), FIG. 1341 (SEQ ID NO:1341), FIG. 1343 (SEQ ID NO:1343), FIG. 1345 (SEQ ID NO:1345), FIG. 1347 (SEQ ID NO:1347), FIG. 1349 A-B (SEQ ID NO:1349), FIG. 1351 (SEQ ID NO:1351), FIG. 1353 (SEQ ID NO:1353), FIG. 1355 A-B (SEQ ID NO:1355), FIG. 1357 (SEQ ID NO:1357), FIG. 1359 (SEQ ID NO:1359), FIG. 1360 (SEQ ID NO:1360), FIG. 1362 (SEQ ID NO:1362), FIG. 1364 (SEQ ID NO:1364), FIG. 1366 (SEQ ID NO:1366), FIG. 1368 (SEQ ID NO:1368), FIG. 1370 (SEQ ID NO:1370), FIG. 1372 (SEQ ID NO:1372), FIG. 1374 (SEQ ID NO:1374), FIG. 1376 (SEQ ID NO:1376), FIG. 1378 (SEQ ID NO:1378), FIG. 1380 A-B (SEQ ID NO:1380), FIG. 1382 (SEQ ID NO:1382), FIG. 1384 (SEQ ID NO:1384), FIG. 1386 (SEQ ID NO:1386), FIG. 1388 (SEQ ID NO:1388), FIG. 1390 A-B (SEQ ID NO:1390), FIG. 1392 (SEQ ID NO:1392), FIG. 1394 (SEQ ID NO:1394), FIG. 1396 (SEQ ID NO:1396), FIG. 1398 A-B (SEQ ID NO:1398), FIG. 1400 (SEQ ID NO:1400), FIG. 1402 (SEQ ID NO:1402), FIG. 1404 (SEQ ID NO:1404), FIG. 1406 (SEQ ID NO:1406), FIG. 1408 (SEQ ID NO:1408), FIG. 1410 (SEQ ID NO:1410), FIG. 1412 (SEQ ID NO:1412), FIG. 1414 (SEQ ID NO:1414), FIG. 1416 (SEQ ID NO:1416), FIG. 1418 (SEQ ID NO:1418), FIG. 1420 (SEQ ID NO:1420), FIG. 1422 (SEQ ID NO:1422), FIG. 1424 (SEQ ID NO:1424), FIG. 1426 (SEQ ID NO:1426), FIG. 1428 (SEQ ID NO:1428), FIG. 1430 (SEQ ID NO:1430), FIG. 1432 A-B (SEQ ID NO:1432), FIG. 1434 (SEQ ID NO:1434), FIG. 1436 A-B (SEQ ID NO:1436), FIG. 1438 (SEQ ID NO:1438), FIG. 1440 (SEQ ID NO:1440), FIG. 1442 A-B (SEQ ID NO:1442), FIG. 1444 A-B (SEQ ID NO:1444), FIG. 1446 (SEQ ID NO:1446), FIG. 1448 (SEQ ID NO:1448), FIG. 1450 (SEQ ID NO:1450), FIG. 1452 (SEQ ID NO:1452), FIG. 1454 (SEQ ID NO:1454), FIG. 1456 (SEQ ID NO:1456), FIG. 1458 (SEQ ID NO:1458), FIG. 1460 (SEQ ID NO:1460), FIG. 1462 (SEQ ID NO:1462), FIG. 1464 (SEQ ID NO:1464), FIG. 1466 (SEQ ID NO:1466), FIG. 1468 (SEQ ID NO:1468), FIG. 1470 (SEQ ID NO:1470), FIG. 1472 (SEQ ID NO:1472), FIG. 1474 (SEQ ID NO:1474), FIG. 1476 (SEQ ID NO:1476), FIG. 1478 (SEQ ID NO:1478), FIG. 1480 (SEQ ID NO:1480), FIG. 1482 (SEQ ID NO:1482), FIG. 1484 (SEQ ID NO:1484), FIG. 1486 (SEQ ID NO:1486), FIG. 1488A-B (SEQ ID NO:1488), FIG. 1490 (SEQ ID NO:1490), FIG. 1492 (SEQ ID NO:1492), FIG. 1494 (SEQ ID NO:1494), FIG. 1496 (SEQ ID NO:1496), FIG. 1498 (SEQ ID NO:1498), FIG. 1500 (SEQ ID NO:1500), FIG. 1502 (SEQ ID NO:1502), FIG. 1504 (SEQ ID NO:1504), FIG. 1506 (SEQ ID NO:1506), FIG. 1508 (SEQ ID NO:1508), FIG. 1510 (SEQ ID NO:1510), FIG. 1512 (SEQ ID NO:1512), FIG. 1514 (SEQ ID NO:1514), FIG. 1516 (SEQ ID NO:1516), FIG. 1518 (SEQ ID NO:1518), FIG. 1520 (SEQ ID NO:1520), FIG. 1522 (SEQ ID NO:1522), FIG. 1524 (SEQ ID NO:1524), FIG. 1526 (SEQ ID NO:1526), FIG. 1528 (SEQ ID NO:1528), FIG. 1530 (SEQ ID NO:1530), FIG. 1532 (SEQ ID NO:1532), FIG. 1534 (SEQ ID NO:1534), FIG. 1536 (SEQ ID NO:1536), FIG. 1538 (SEQ ID NO:1538), FIG. 1540 (SEQ ID NO:1540), FIG. 1542 (SEQ ID NO:1542), FIG. 1544 (SEQ ID NO:1544), FIG. 1546 (SEQ ID NO:1546), FIG. 1548 (SEQ ID NO:1548), FIG. 1550 (SEQ ID NO:1550), FIG. 1552 (SEQ ID NO:1552), FIG. 1554 (SEQ ID NO:1554), FIG. 1556 (SEQ ID NO:1556), FIG. 1558 A-B (SEQ ID NO:1558), FIG. 1560 (SEQ ID NO:1560), FIG. 1562 (SEQ ID NO:1562), FIG. 1564 (SEQ ID NO:1564), FIG. 1566 (SEQ ID NO:1566), FIG. 1568 (SEQ ID NO:1568), FIG. 1570 A-B (SEQ ID NO:1570), FIG. 1572A-C (SEQ ID NO:1572), FIG. 1574 (SEQ ID NO:1574), FIG. 1576 (SEQ ID NO:1576), FIG. 1577 (SEQ ID NO:1577), FIG. 1579 (SEQ ID NO:1579), FIG. 1580 (SEQ ID NO:1580), FIG. 1582 A-B (SEQ ID NO:1582), FIG. 1584 (SEQ ID NO:1584), FIG. 1586 (SEQ ID NO:1586), FIG. 1588 A-B (SEQ ID NO:1588), FIG. 1590 (SEQ ID NO:1590), FIG. 1591 (SEQ ID NO:1591), FIG. 1593 (SEQ ID NO:1593), FIG. 1595 (SEQ ID NO:1595), FIG. 1597 (SEQ ID NO:1597), FIG. 1599A-D (SEQ ID NO:1599), FIG. 1601A-E (SEQ ID NO:1601), FIG. 1603 A-B (SEQ ID NO:1603), FIG. 1605 A-B (SEQ ID NO:1605), FIG. 1607 (SEQ ID NO:1607), FIG. 1608 (SEQ ID NO:1608), FIG. 1610 (SEQ ID NO:1610), FIG. 1612 (SEQ ID NO:1612), FIG. 1613 A-B (SEQ ID NO:1613), FIG. 1615 (SEQ ID NO:1615), FIG. 1616 (SEQ ID NO:1616), FIG. 1618 (SEQ ID NO:1618), FIG. 1620 A-B (SEQ ID NO:1620), FIG. 1622 (SEQ ID NO:1622), FIG. 1624 (SEQ ID NO:1624), FIG. 1625 (SEQ ID NO:1625), FIG. 1627 (SEQ ID NO:1627), FIG. 1629 (SEQ ID NO:1629), FIG. 1631 (SEQ ID NO:1631), FIG. 1633 (SEQ ID NO:1633), FIG. 1634 (SEQ ID NO:1634), FIG. 1635 (SEQ ID NO:1635), FIG. 1637 (SEQ ID NO:1637), FIG. 1638 (SEQ ID NO:1638), FIG. 1640 (SEQ ID NO:1640), FIG. 1641 (SEQ ID NO:1641), FIG. 1642 (SEQ ID NO:1642), FIG. 1643 (SEQ ID NO:1643), FIG. 1645 (SEQ ID NO:1645), FIG. 1647 A-B (SEQ ID NO:1647), FIG. 1649 (SEQ ID NO:1649), FIG. 1651 (SEQ ID NO:1651), FIG. 1653 (SEQ ID NO:1653), FIG. 1655 (SEQ ID NO:1655), FIG. 1657 (SEQ ID NO:1657), FIG. 1659 (SEQ ID NO:1659), FIG. 1661 (SEQ ID NO:1661), FIG. 1663 (SEQ ID NO:1663), FIG. 1665 (SEQ ID NO:1665), FIG. 1667 (SEQ ID NO:1667), FIG. 1669 (SEQ ID NO:1669), FIG. 1671 (SEQ ID NO:1671), FIG. 1673 (SEQ ID NO:1673), FIG. 1675 (SEQ ID NO:1675), FIG. 1677 (SEQ ID NO:1677), FIG. 1679 A-B (SEQ ID NO:1679), FIG. 1681 (SEQ ID NO:1681), FIG. 1682A-B (SEQ ID NO:1682), FIG. 1684 A-B (SEQ ID NO:1684), FIG. 1686 A-B (SEQ ID NO:1686), FIG. 1688 (SEQ ID NO:1688), FIG. 1690 (SEQ ID NO:1690), FIG. 1691 (SEQ ID NO:1691), FIG. 1693 (SEQ ID NO:1693), FIG. 1695 (SEQ ID NO:1695), FIG. 1697 (SEQ ID NO:1697), FIG. 1699 (SEQ ID NO:1699), FIG. 1700 (SEQ ID NO:1700), FIG. 1702 (SEQ ID NO:1702), FIG. 1704 (SEQ ID NO:1704), FIG. 1706 (SEQ ID NO:1706), FIG. 1708A-C (SEQ ID NO:1708), FIG. 1710 (SEQ ID NO:1710), FIG. 1712 (SEQ ID NO:1712), FIG. 1714 (SEQ ID NO:1714), FIG. 1716 (SEQ ID NO:1716), FIG. 1718 (SEQ ID NO:1718), FIG. 1720A-C (SEQ ID NO:1720), FIG. 1722 (SEQ ID NO:1722), FIG. 1724A-B (SEQ ID NO:1724), FIG. 1726 (SEQ ID NO:1726), FIG. 1728A-B (SEQ ID NO:1728), FIG. 1730 (SEQ ID NO:1730), FIG. 1731 (SEQ ID NO:1731), FIG. 1733 (SEQ ID NO:1733), FIG. 1735 (SEQ ID NO:1735), FIG. 1737 (SEQ ID NO:1737), FIG. 1739 (SEQ ID NO:1739), FIG. 1740 (SEQ ID NO:1740), FIG. 1742 (SEQ ID NO:1742), FIG. 1744 (SEQ ID NO:1744), FIG. 1746 (SEQ ID NO:1746), FIG. 1748 (SEQ ID NO:1748), FIG. 1750 (SEQ ID NO:1750), FIG. 1752A-B (SEQ ID NO:1752), FIG. 1754 (SEQ ID NO:1754), FIG. 1756 (SEQ ID NO:1756), FIG. 1758 (SEQ ID NO:1758), FIG. 1759 (SEQ ID NO:1759), FIG. 1761 (SEQ ID NO:1761), FIG. 1763 (SEQ ID NO:1763), FIG. 1765 (SEQ ID NO:1765), FIG. 1767 (SEQ ID NO:1767), FIG. 1768 (SEQ ID NO:1768), FIG. 1770 (SEQ ID NO:1770), FIG. 1772 (SEQ ID NO:1772), FIG. 1774 (SEQ ID NO:1774), FIG. 1776 (SEQ ID NO:1776), FIG. 1778 (SEQ ID NO:1778), FIG. 1780 (SEQ ID NO:1780), FIG. 1782 (SEQ ID NO:1782), FIG. 1784 (SEQ ID NO:1784), FIG. 1786 (SEQ ID NO:1786), FIG. 1788 (SEQ ID NO:1788), FIG. 1790 (SEQ ID NO:1790), FIG. 1791 (SEQ ID NO:1791), FIG. 1793 (SEQ ID NO:1793), FIG. 1794 (SEQ ID NO:1794), FIG. 1796 (SEQ ID NO:1796), FIG. 1798 (SEQ ID NO:1798), FIG. 1800 (SEQ ID NO:1800), FIG. 1801 (SEQ ID NO:1801), FIG. 1803 (SEQ ID NO:1803), FIG. 1804 (SEQ ID NO:1804), FIG. 1806 (SEQ ID NO:1806), FIG. 1808 (SEQ ID NO:1808), FIG. 1810 (SEQ ID NO:1810), FIG. 1812A-B (SEQ ID NO:1812), FIG. 1814 (SEQ ID NO:1814), FIG. 1816 (SEQ ID NO:1816), FIG. 1818 (SEQ ID NO:1818), FIG. 1820 (SEQ ID NO:1820), FIG. 1822 (SEQ ID NO:1822), FIG. 1824A-B (SEQ ID NO:1824), FIG. 1826 (SEQ ID NO:1826), FIG. 1828 (SEQ ID NO:1828), FIG. 1830 (SEQ ID NO:1830), FIG. 1832 (SEQ ID NO:1832), FIG. 1834 (SEQ ID NO:1834), FIG. 1835 (SEQ ID NO:1835), FIG. 1837 (SEQ ID NO:1837), FIG. 1839 (SEQ ID NO:1839), FIG. 1840 (SEQ ID NO:1840), FIG. 1842 (SEQ ID NO:1842), FIG. 1844A-B (SEQ ID NO:1844), FIG. 1846 (SEQ ID NO:1846), FIG. 1848A-B (SEQ ID NO:1848), FIG. 1850 (SEQ ID NO:1850), FIG. 1852 (SEQ ID NO:1852), FIG. 1853A-B (SEQ ID NO:1853), FIG. 1855 (SEQ ID NO:1855), FIG. 1857 (SEQ ID NO:1857), FIG. 1859 (SEQ ID NO:1859), FIG. 1861 (SEQ ID NO:1861), FIG. 1862A-B (SEQ ID NO:1862), FIG. 1864 (SEQ ID NO:1864), FIG. 1866 (SEQ ID NO:1866), FIG. 1868A-B (SEQ ID NO:1868), FIG. 1870 (SEQ ID NO:1870), FIG. 1872 (SEQ ID NO:1872), FIG. 1874 (SEQ ID NO:1874), FIG. 1876 (SEQ ID NO:1876), FIG. 1878 (SEQ ID NO:1878), FIG. 1879 (SEQ ID NO:1879), FIG. 1880 (SEQ ID NO:1880), FIG. 1882 (SEQ ID NO:1882), FIG. 1884 (SEQ ID NO:1884), FIG. 1886A-B (SEQ ID NO:1886), FIG. 1888 (SEQ ID NO:1888), FIG. 1890 (SEQ ID NO:1890), FIG. 1892 (SEQ ID NO:1892), FIG. 1894 (SEQ ID NO:1894), FIG. 1896 (SEQ ID NO:1896), FIG. 1898A-B (SEQ ID NO:1898), FIG. 1900 (SEQ ID NO:1900), FIG. 1902 (SEQ ID NO:1902), FIG. 1904 (SEQ ID NO:1904), FIG. 1906A-B (SEQ ID NO:1906), FIG. 1907 (SEQ ID NO:1907), FIG. 1909 (SEQ ID NO:1909), FIG. 1911 (SEQ ID NO:1911), FIG. 1912 (SEQ ID NO:1912), FIG. 1914 (SEQ ID NO:1914), FIG. 1916A-B (SEQ ID NO:1916), FIG. 1918 (SEQ ID NO:1918), FIG. 1920A-B (SEQ ID NO:1920), FIG. 1921 (SEQ ID NO:1921), FIG. 1923 (SEQ ID NO:1923), FIG. 1925 (SEQ ID NO:1925), FIG. 1927 (SEQ ID NO:1927), FIG. 1929 (SEQ ID NO:1929), FIG. 1931 (SEQ ID NO:1931), FIG. 1933 (SEQ ID NO:1933), FIG. 1935 (SEQ ID NO:1935), FIG. 1937 (SEQ ID NO:1937), FIG. 1939 (SEQ ID NO:1939), FIG. 1941 (SEQ ID NO:1941), FIG. 1943 (SEQ ID NO:1943), FIG. 1945 (SEQ ID NO:1945), FIG. 1947 (SEQ ID NO:1947), FIG. 1949 (SEQ ID NO:1949), FIG. 1951 (SEQ ID NO:1951), FIG. 1953 (SEQ ID NO:1953), FIG. 1955 (SEQ ID NO:1955), FIG. 1957 (SEQ ID NO:1957), FIG. 1959 (SEQ ID NO:1959), FIG. 1961 (SEQ ID NO:1961), FIG. 1963A-B (SEQ ID NO:1963), FIG. 1965 (SEQ ID NO:1965), FIG. 1967 (SEQ ID NO:1967), FIG. 1969 (SEQ ID NO:1969), FIG. 1971 (SEQ ID NO:1971), FIG. 1973 (SEQ ID NO:1973), FIG. 1975 (SEQ ID NO:1975), FIG. 1977 (SEQ ID NO:1977), FIG. 1979 (SEQ ID NO:1979), FIG. 1981 (SEQ ID NO:1981), FIG. 1983 (SEQ ID NO:1983), FIG. 1985 (SEQ ID NO:1985), FIG. 1987 (SEQ ID NO:1987), FIG. 1989 (SEQ ID NO:1989), FIG. 1991 (SEQ ID NO:1991), FIG. 1993 (SEQ ID NO:1993), FIG. 1995 (SEQ ID NO:1995), FIG. 1997 (SEQ ID NO:1997), FIG. 1999 (SEQ ID NO:1999), FIG. 2001 (SEQ ID NO:2001), FIG. 2003 (SEQ ID NO:2003), FIG. 2005A-B (SEQ ID NO:2005), FIG. 2007 (SEQ ID NO:2007), FIG. 2009 (SEQ ID NO:2009), FIG. 2011 (SEQ ID NO:2011), FIG. 2013 (SEQ ID NO:2013), FIG. 2015 (SEQ ID NO:2015), FIG. 2017A-B (SEQ ID NO:2017), FIG. 2019A-B (SEQ ID NO:2019), FIG. 2021 (SEQ ID NO:2021), FIG. 2023 (SEQ ID NO:2023), FIG. 2025 (SEQ ID NO:2025), FIG. 2027 (SEQ ID NO:2027), FIG. 2029 (SEQ ID NO:2029), FIG. 2031 (SEQ ID NO:2031), FIG. 2033 (SEQ ID NO:2033), FIG. 2035 (SEQ ID NO:2035), FIG. 2037 (SEQ ID NO:2037), FIG. 2039 (SEQ ID NO:2039), FIG. 2041 (SEQ ID NO:2041), FIG. 2043 (SEQ ID NO:2043), FIG. 2045 (SEQ ID NO:2045), FIG. 2047 (SEQ ID NO:2047), FIG. 2049A-B (SEQ ID NO:2049), FIG. 2051 (SEQ ID NO:2051), FIG. 2053 (SEQ ID NO:2053), FIG. 2055A-B (SEQ ID NO:2055), FIG. 2057 (SEQ ID NO:2057), FIG. 2059 (SEQ ID NO:2059), FIG. 2061 (SEQ ID NO:2061), FIG. 2063 (SEQ ID NO:2063), FIG. 2065 (SEQ ID NO:2065), FIG. 2067 (SEQ ID NO:2067), FIG. 2069 (SEQ ID NO:2069), FIG. 2071A-B (SEQ ID NO:2071), FIG. 2073 (SEQ ID NO:2073), FIG. 2075 (SEQ ID NO:2075), FIG. 2077 (SEQ ID NO:2077), FIG. 2079 (SEQ ID NO:2079), FIG. 2081 (SEQ ID NO:2081), FIG. 2083 (SEQ ID NO:2083), FIG. 2085 (SEQ ID NO:2085), FIG. 2087 (SEQ ID NO:2087), FIG. 2089 (SEQ ID NO:2089), FIG. 2091 (SEQ ID NO:2091), FIG. 2093 (SEQ ID NO:2093), FIG. 2095 (SEQ ID NO:2095), FIG. 2097 (SEQ ID NO:2097), FIG. 2099 (SEQ ID NO:2099), FIG. 2101 (SEQ ID NO:2101), FIG. 2103 (SEQ ID NO:2103), FIG. 2105 (SEQ ID NO:2105), FIG. 2107 (SEQ ID NO:2107), FIG. 2109 (SEQ ID NO:2109), FIG. 2111 (SEQ ID NO:2111), FIG. 2113 (SEQ ID NO:2113), FIG. 2115 (SEQ ID NO:2115), FIG. 2117 (SEQ ID NO:2117), FIG. 2119 (SEQ ID NO:2119), FIG. 2121 (SEQ ID NO:2121), FIG. 2123 (SEQ ID NO:2123), FIG. 2125 (SEQ ID NO:2125), FIG. 2127 (SEQ ID NO:2127), FIG. 2129 (SEQ ID NO:2129), FIG. 2131 (SEQ ID NO:2131), FIG. 2133 (SEQ ID NO:2133), FIG. 2135 (SEQ ID NO:2135), FIG. 2137 (SEQ ID NO:2137), FIG. 2139 (SEQ ID NO:2139), FIG. 2141 (SEQ ID NO:2141), FIG. 2143 (SEQ ID NO:2143), FIG. 2145A-B (SEQ ID NO:2145), FIG. 2147 (SEQ ID NO:2147), FIG. 2149 (SEQ ID NO:2149), FIG. 2151 (SEQ ID NO:2151), FIG. 2153 (SEQ ID NO:2153), FIG. 2155 (SEQ ID NO:2155), FIG. 2157 (SEQ ID NO:2157), FIG. 2159 (SEQ ID NO:2159), FIG. 2161A-B (SEQ ID NO:2161), FIG. 2162 (SEQ ID NO:2162), FIG. 2164 (SEQ ID NO:2164), FIG. 2166A-B (SEQ ID NO:2166), FIG. 2168 (SEQ ID NO:2168), FIG. 2169 (SEQ ID NO:2169), FIG. 2171A-B (SEQ ID NO:2171), FIG. 2173 (SEQ ID NO:2173), FIG. 2175A-B (SEQ ID NO:2175), FIG. 2177 (SEQ ID NO:2177), FIG. 2179 (SEQ ID NO:2179), FIG. 2181 (SEQ ID NO:2181), FIG. 2183 (SEQ ID NO:2183), FIG. 2185A-B (SEQ ID NO:2185), FIG. 2187 (SEQ ID NO:2187), FIG. 2189 (SEQ ID NO:2189), FIG. 2190A-B (SEQ ID NO:2190), FIG. 2192 (SEQ ID NO:2192), FIG. 2194 (SEQ ID NO:2194), FIG. 2196 (SEQ ID NO:2196), FIG. 2198 (SEQ ID NO:2198), FIG. 2200 (SEQ ID NO:2200), FIG. 2202 (SEQ ID NO:2202), FIG. 2203 (SEQ ID NO:2203), FIG. 2205 (SEQ ID NO:2205), FIG. 2206A-B (SEQ ID NO:2206), FIG. 2208 (SEQ ID NO:2208), FIG. 2210 (SEQ ID NO:2210), FIG. 2211 (SEQ ID NO:2211), FIG. 2213 (SEQ ID NO:2213), FIG. 2215 (SEQ ID NO:2215), FIG. 2217 (SEQ ID NO:2217), FIG. 2219 (SEQ ID NO:2219), FIG. 2221 (SEQ ID NO:2221), FIG. 2223 (SEQ ID NO:2223), FIG. 2225 (SEQ ID NO:2225), FIG. 2227 (SEQ ID NO:2227), FIG. 2229 (SEQ ID NO:2229), FIG. 2231 (SEQ ID NO:2231), FIG. 2233 (SEQ ID NO:2233), FIG. 2235 (SEQ ID NO:2235), FIG. 2237 (SEQ ID NO:2237), FIG. 2239 (SEQ ID NO:2239), FIG. 2241 (SEQ ID NO:2241), FIG. 2243 (SEQ ID NO:2243), FIG. 2245 (SEQ ID NO:2245), FIG. 2247 (SEQ ID NO:2247), FIG. 2249 (SEQ ID NO:2249), FIG. 2251 (SEQ ID NO:2251), FIG. 2253 (SEQ ID NO:2253), FIG. 2255 (SEQ ID NO:2255), FIG. 2257 (SEQ ID NO:2257), FIG. 2259 (SEQ ID NO:2259), FIG. 2261 (SEQ ID NO:2261), FIG. 2263 (SEQ ID NO:2263), FIG. 2265 (SEQ ID NO:2265), FIG. 2267 (SEQ ID NO:2267), FIG. 2269 (SEQ ID NO:2269), FIG. 2271 (SEQ ID NO:2271), FIG. 2273 (SEQ ID NO:2273), FIG. 2275 (SEQ ID NO:2275), FIG. 2277 (SEQ ID NO:2277), FIG. 2279 (SEQ ID NO:2279), FIG. 2281 (SEQ ID NO:2281), FIG. 2283 (SEQ ID NO:2283), FIG. 2285 (SEQ ID NO:2285), FIG. 2287 (SEQ ID NO:2287), FIG. 2289 (SEQ ID NO:2289), FIG. 2291 (SEQ ID NO:2291), FIG. 2293 (SEQ ID NO:2293), FIG. 2295 (SEQ ID NO:2295), FIG. 2297 (SEQ ID NO:2297), FIG. 2299 (SEQ ID NO:2299), FIG. 2300 (SEQ ID NO:2300), FIG. 2302A-B (SEQ ID NO:2302), FIG. 2304 (SEQ ID NO:2304), FIG. 2306 (SEQ ID NO:2306), FIG. 2308 (SEQ ID NO:2308), FIG. 2309 (SEQ ID NO:2309), FIG. 2311 (SEQ ID NO:2311), FIG. 2313 (SEQ ID NO:2313), FIG. 2315 (SEQ ID NO:2315), FIG. 2317 (SEQ ID NO:2317), FIG. 2318 (SEQ ID NO:2318), FIG. 2320A-B (SEQ ID NO:2320), FIG. 2322 (SEQ ID NO:2322), FIG. 2324 (SEQ ID NO:2324), FIG. 2326 (SEQ ID NO:2326), FIG. 2328 (SEQ ID NO:2328), FIG. 2330A-B (SEQ ID NO:2330), FIG. 2332 (SEQ ID NO:2332), FIG. 2334 (SEQ ID NO:2334), FIG. 2336A-B (SEQ ID NO:2336), FIG. 2338 (SEQ ID NO:2338), FIG. 2340 (SEQ ID NO:2340), FIG. 2342 (SEQ ID NO:2342), FIG. 2344 (SEQ ID NO:2344), FIG. 2346 (SEQ ID NO:2346), FIG. 2348 (SEQ ID NO:2348), FIG. 2350 (SEQ ID NO:2350), FIG. 2352 (SEQ ID NO:2352), FIG. 2354A-B (SEQ ID NO:2354), FIG. 2356 (SEQ ID NO:2356), FIG. 2358 (SEQ ID NO:2358), FIG. 2360 (SEQ ID NO:2360), FIG. 2362 (SEQ ID NO:2362), FIG. 2364 (SEQ ID NO:2364), FIG. 2366 (SEQ ID NO:2366), FIG. 2368 (SEQ ID NO:2368), FIG. 2370 (SEQ ID NO:2370), FIG. 2372 (SEQ ID NO:2372), FIG. 2374 (SEQ ID NO:2374), FIG. 2376A-B (SEQ ID NO:2376), FIG. 2377 (SEQ ID NO:2377), FIG. 2379 (SEQ ID NO:2379), FIG. 2381 (SEQ ID NO:2381), FIG. 2382 (SEQ ID NO:2382), FIG. 2384 (SEQ ID NO:2384), FIG. 2385 (SEQ ID NO:2385), FIG. 2387 (SEQ ID NO:2387), FIG. 2389 (SEQ ID NO:2389), FIG. 2390 (SEQ ID NO:2390), FIG. 2392 (SEQ ID NO:2392), FIG. 2394 (SEQ ID NO:2394), FIG. 2395A-B (SEQ ID NO:2395), FIG. 2397 (SEQ ID NO:2397), FIG. 2399 (SEQ ID NO:2399), FIG. 2400 (SEQ ID NO:2400), FIG. 2401 (SEQ ID NO:2401), FIG. 2403 (SEQ ID NO:2403), FIG. 2405 (SEQ ID NO:2405), FIG. 2406 (SEQ ID NO:2406), FIG. 2407 (SEQ ID NO:2407), FIG. 2409 (SEQ ID NO:2409), FIG. 2411 (SEQ ID NO:2411), FIG. 2413 (SEQ ID NO:2413), FIG. 2414 (SEQ ID NO:2414), FIG. 2416 (SEQ ID NO:2416), FIG. 2417 (SEQ ID NO:2417), FIG. 2419 (SEQ ID NO:2419), FIG. 2421 (SEQ ID NO:2421), FIG. 2423 (SEQ ID NO:2423), FIG. 2425 (SEQ ID NO:2425), FIG. 2427 (SEQ ID NO:2427), FIG. 2428 (SEQ ID NO:2428), FIG. 2429 (SEQ ID NO:2429), FIG. 2431 (SEQ ID NO:2431), FIG. 2433 (SEQ ID NO:2433), FIG. 2435 (SEQ ID NO:2435), FIG. 2437 (SEQ ID NO:2437), FIG. 2439 (SEQ ID NO:2439) or FIG. 2441 (SEQ ID NO:2441).

4. A vector comprising the nucleic acid of claim 1.

5. The vector of claim 4 operably linked to control sequences recognized by a host cell transformed with the vector.

6. A host cell comprising the vector of claim 4.

7. The host cell of claim 6, wherein said cell is a CHO cell, an E. coli cell or a yeast cell.

8. A process for producing a PRO polypeptide comprising culturing the host cell of claim 7 under conditions suitable for expression of said PRO polypeptide and recovering said PRO polypeptide from the cell culture.

9. An isolated polypeptide having at least 80% amino acid sequence identity to an amino acid sequence of the polypeptide shown in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440) or FIG. 2442 (SEQ ID NO:2442).

10. A chimeric molecule comprising a polypeptide according to claim 9 fused to a heterologous amino acid sequence.

11. The chimeric molecule of claim 10, wherein said heterologous amino acid sequence is an epitope tag sequence or an Fc region of an immunoglobulin.

12. An antibody which specifically binds to a polypeptide according to claim 9.

13. The antibody of claim 12, wherein said antibody is a monoclonal antibody, a humanized antibody or a single-chain antibody.

14. A composition of matter comprising (a) a polypeptide of claim 9, (b) an agonist of said polypeptide, (c) an antagonist of said polypeptide, or (d) an antibody that binds to said polypeptide, in combination with a carrier.

15. The composition of matter of claim 14, wherein said carrier is a pharmaceutically acceptable carrier.

16. The composition of matter of claim 15 comprising a therapeutically effective amount of (a), (b), (c) or (d).

17. An article of manufacture, comprising:
a container;
a label on said container; and
a composition of matter comprising (a) a polypeptide of claim 9, (b) an agonist of said polypeptide, (c) an antagonist of said polypeptide, or (d) an antibody that binds to said polypeptide, contained within said container, wherein label on said container indicates that said composition of matter can be used for treating an immune related disease.

18. A method of treating an immune related disorder in a mammal in need thereof comprising administering to said mammal a therapeutically effective amount of (a) a polypeptide of claim 9, (b) an agonist of said polypeptide, (c) an antagonist of said polypeptide, or (d) an antibody that binds to said polypeptide.

19. The method of claim 18, wherein the immune related disorder is systemic lupus erythematosis, rheumatoid arthritis, osteoarthritis, juvenile chronic arthritis, a spondyloarthropathy, systemic sclerosis, an idiopathic inflammatory myopathy, Sjögren's syndrome, systemic vasculitis, sarcoidosis, autoimmune hemolytic anemia, autoimmune thrombocytopenia, thyroiditis, diabetes mellitus, immune-mediated renal disease, a demyelinating disease of the central or peripheral nervous system, idiopathic demyelinating polyneuropathy, Guillain-Barré syndrome, a chronic inflammatory demyelinating polyneuropathy, a hepatobiliary disease, infectious or autoimmune chronic active hepatitis, primary biliary cirrhosis, granulomatous hepatitis, sclerosing cholangitis, inflammatory bowel disease, gluten-sensitive enteropathy, Whipple's disease, an autoimmune or immune-mediated skin disease, a bullous skin disease, erythema multiforme, contact dermatitis, psoriasis, an allergic disease, asthma, allergic rhinitis, atopic dermatitis, food hypersensitivity, urticaria, an immunologic disease of the lung, eosinophilic pneumonias, idiopathic pulmonary fibrosis, hypersensitivity pneumonitis, a transplantation associated disease, graft rejection or graft-versus-host-disease.

20. A method for determining the presence of a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:310), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130). FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442), in a sample suspected of containing said polypeptide, said method comprising exposing said sample to an anti-PRO antibody, and determining binding of said antibody to a component of said sample.

21. A method of diagnosing an immune related disease in a mammal, said method comprising detecting the level of expression of a gene encoding a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947). FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442), (a) in a test sample of tissue cells obtained from the mammal, and (b) in a control sample of known normal tissue cells of the same cell type, wherein a higher or lower level of expression of said gene in the test sample as compared to the control sample is indicative of the presence of an immune related disease in the mammal from which the test tissue cells were obtained.

22. A method of diagnosing an immune related disease in a mammal, said method comprising (a) contacting a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442) anti-PRO antibody with a test sample of tissue cells obtained from said mammal and (b) detecting the formation of a complex between the antibody and the polypeptide in the test sample, wherein formation of said complex is indicative of the presence of an immune related disease in the mammal from which the test tissue cells were obtained.

23. A method of identifying a compound that inhibits the activity of a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2774), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442) said method comprising contacting cells which normally respond to said polypeptide with (a) said polypeptide and (b) a candidate compound, and determining the lack responsiveness by said cell to (a).

24. A method of identifying a compound that inhibits the expression of a gene encoding a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 A (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440) or FIG. 2442 (SEQ ID NO:2442) said method comprising contacting cells which normally express said polypeptide with a candidate compound, and determining the lack of expression said gene.

25. The method of claim 24, wherein said candidate compound is an antisense nucleic acid.

26. A method of identifying a compound that mimics the activity of a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1155), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442) said method comprising contacting cells which normally respond to said polypeptide with a candidate compound, and determining the responsiveness by said cell to said candidate compound.

27. A method of stimulating the immune response in a mammal, said method comprising administering to said mammal an effective amount of a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767), FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:982), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442) antagonist, wherein said immune response is stimulated.

28. A method of diagnosing an inflammatory immune response in a mammal, said method comprising detecting the level of expression of a gene encoding a PRO polypeptide of the invention as described in FIG. 2 (SEQ ID NO:2), FIG. 4 (SEQ ID NO:4), FIG. 6 (SEQ ID NO:6), FIG. 8 (SEQ ID NO:8), FIG. 10 (SEQ ID NO:10), FIG. 12 (SEQ ID NO:12), FIG. 14 (SEQ ID NO:14), FIG. 16 (SEQ ID NO:16), FIG. 18 (SEQ ID NO:18), FIG. 20 (SEQ ID NO:20), FIG. 22 (SEQ ID NO:22), FIG. 24 (SEQ ID NO:24), FIG. 26 (SEQ ID NO:26), FIG. 28 (SEQ ID NO:28), FIG. 30 (SEQ ID NO:30), FIG. 32 (SEQ ID NO:32), FIG. 34 (SEQ ID NO:34), FIG. 36 (SEQ ID NO:36), FIG. 38 (SEQ ID NO:38), FIG. 40 (SEQ ID NO:40), FIG. 42 (SEQ ID NO:42), FIG. 44 (SEQ ID NO:44), FIG. 46 (SEQ ID NO:46), FIG. 48 (SEQ ID NO:48), FIG. 50 (SEQ ID NO:50), FIG. 52 (SEQ ID NO:52), FIG. 54 (SEQ ID NO:54), FIG. 56 (SEQ ID NO:56), FIG. 58 (SEQ ID NO:58), FIG. 60 (SEQ ID NO:60), FIG. 62 (SEQ ID NO:62), FIG. 64 (SEQ ID NO:64), FIG. 66 (SEQ ID NO:66), FIG. 68 (SEQ ID NO:68), FIG. 70 (SEQ ID NO:70), FIG. 72 (SEQ ID NO:72), FIG. 74 (SEQ ID NO:74), FIG. 76 (SEQ ID NO:76), FIG. 78 (SEQ ID NO:78), FIG. 80 (SEQ ID NO:80), FIG. 82 (SEQ ID NO:82), FIG. 84 (SEQ ID NO:84), FIG. 86 (SEQ ID NO:86), FIG. 88 (SEQ ID NO:88), FIG. 90 (SEQ ID NO:90), FIG. 92 (SEQ ID NO:92), FIG. 94 (SEQ ID NO:94), FIG. 96 (SEQ ID NO:96), FIG. 98 (SEQ ID NO:98), FIG. 100 (SEQ ID NO:100), FIG. 102 (SEQ ID NO:102), FIG. 104 (SEQ ID NO:104), FIG. 106 (SEQ ID NO:106), FIG. 108 (SEQ ID NO:108), FIG. 110 (SEQ ID NO:110), FIG. 112 (SEQ ID NO:112), FIG. 114 (SEQ ID NO:114), FIG. 116 (SEQ ID NO:116), FIG. 118 (SEQ ID NO:118), FIG. 121 (SEQ ID NO:121), FIG. 123 (SEQ ID NO:123), FIG. 125 (SEQ ID NO:125), FIG. 127 (SEQ ID NO:127), FIG. 129 (SEQ ID NO:129), FIG. 131 (SEQ ID NO:131), FIG. 133 (SEQ ID NO:133), FIG. 135 (SEQ ID NO:135), FIG. 137 (SEQ ID NO:137), FIG. 139 (SEQ ID NO:139), FIG. 141 (SEQ ID NO:141), FIG. 143 (SEQ ID NO:143), FIG. 145 (SEQ ID NO:145), FIG. 147 (SEQ ID NO:147), FIG. 149 (SEQ ID NO:149), FIG. 151 (SEQ ID NO:151), FIG. 153 (SEQ ID NO:153), FIG. 155 (SEQ ID NO:155), FIG. 157 (SEQ ID NO:157), FIG. 159 (SEQ ID NO:159), FIG. 161 (SEQ ID NO:161), FIG. 163 (SEQ ID NO:163), FIG. 165 (SEQ ID NO:165), FIG. 167 (SEQ ID NO:167), FIG. 169 (SEQ ID NO:169), FIG. 171 (SEQ ID NO:171), FIG. 173 (SEQ ID NO:173), FIG. 175 (SEQ ID NO:175), FIG. 177 (SEQ ID NO:177), FIG. 179 (SEQ ID NO:179), FIG. 181 (SEQ ID NO:181), FIG. 183 (SEQ ID NO:183), FIG. 185 (SEQ ID NO:185), FIG. 187 (SEQ ID NO:187), FIG. 189 (SEQ ID NO:189), FIG. 191 (SEQ ID NO:191), FIG. 193 (SEQ ID NO:193), FIG. 195 (SEQ ID NO:195), FIG. 197 (SEQ ID NO:197), FIG. 199 (SEQ ID NO:199), FIG. 201 (SEQ ID NO:201), FIG. 203 (SEQ ID NO:203), FIG. 205 (SEQ ID NO:205), FIG. 207 (SEQ ID NO:207), FIG. 209 (SEQ ID NO:209), FIG. 211 (SEQ ID NO:211), FIG. 213 (SEQ ID NO:213), FIG. 215 (SEQ ID NO:215), FIG. 217 (SEQ ID NO:217), FIG. 219 (SEQ ID NO:219), FIG. 221 (SEQ ID NO:221), FIG. 223 (SEQ ID NO:223), FIG. 225 (SEQ ID NO:225), FIG. 227 (SEQ ID NO:227), FIG. 229 (SEQ ID NO:229), FIG. 231 (SEQ ID NO:231), FIG. 233 (SEQ ID NO:233), FIG. 235 (SEQ ID NO:235), FIG. 237 (SEQ ID NO:237), FIG. 239 (SEQ ID NO:239), FIG. 241 (SEQ ID NO:241), FIG. 243 (SEQ ID NO:243), FIG. 245 (SEQ ID NO:245), FIG. 247 (SEQ ID NO:247), FIG. 249 (SEQ ID NO:249), FIG. 251 (SEQ ID NO:251), FIG. 254 (SEQ ID NO:254), FIG. 256 (SEQ ID NO:256), FIG. 258 (SEQ ID NO:258), FIG. 260 (SEQ ID NO:260), FIG. 262 (SEQ ID NO:262), FIG. 264 (SEQ ID NO:264), FIG. 266 (SEQ ID NO:266), FIG. 268 (SEQ ID NO:268), FIG. 270 (SEQ ID NO:270), FIG. 272 (SEQ ID NO:272), FIG. 274 (SEQ ID NO:274), FIG. 276 (SEQ ID NO:276), FIG. 278 (SEQ ID NO:278), FIG. 280 (SEQ ID NO:280), FIG. 282 (SEQ ID NO:282), FIG. 284 (SEQ ID NO:284), FIG. 286 (SEQ ID NO:286), FIG. 288 (SEQ ID NO:288), FIG. 290 (SEQ ID NO:290), FIG. 292 (SEQ ID NO:292), FIG. 294 (SEQ ID NO:294), FIG. 296 (SEQ ID NO:296), FIG. 298 (SEQ ID NO:298), FIG. 300 (SEQ ID NO:300), FIG. 302 (SEQ ID NO:302), FIG. 304 (SEQ ID NO:304), FIG. 306 (SEQ ID NO:306), FIG. 308 (SEQ ID NO:308), FIG. 310 (SEQ ID NO:310), FIG. 312 (SEQ ID NO:312), FIG. 314 (SEQ ID NO:314), FIG. 316 (SEQ ID NO:316), FIG. 318 (SEQ ID NO:318), FIG. 320 (SEQ ID NO:320), FIG. 322 (SEQ ID NO:322), FIG. 324 (SEQ ID NO:324), FIG. 326 (SEQ ID NO:326), FIG. 328 (SEQ ID NO:328), FIG. 330 (SEQ ID NO:330), FIG. 332 (SEQ ID NO:332), FIG. 334 (SEQ ID NO:334), FIG. 336 (SEQ ID NO:336), FIG. 338 (SEQ ID NO:338), FIG. 340 (SEQ ID NO:340), FIG. 342 (SEQ ID NO:342), FIG. 344 (SEQ ID NO:344), FIG. 346 (SEQ ID NO:346), FIG. 348 (SEQ ID NO:348), FIG. 350 (SEQ ID NO:350), FIG. 352 (SEQ ID NO:352), FIG. 354 (SEQ ID NO:354), FIG. 356 (SEQ ID NO:356), FIG. 358 (SEQ ID NO:358), FIG. 360 (SEQ ID NO:360), FIG. 362 (SEQ ID NO:362), FIG. 364 (SEQ ID NO:364), FIG. 366 (SEQ ID NO:366), FIG. 368 (SEQ ID NO:368), FIG. 370 (SEQ ID NO:370), FIG. 372 (SEQ ID NO:372), FIG. 374 (SEQ ID NO:374), FIG. 376 (SEQ ID NO:376), FIG. 378 (SEQ ID NO:378), FIG. 380 (SEQ ID NO:380), FIG. 382 (SEQ ID NO:382), FIG. 384 (SEQ ID NO:384), FIG. 386 (SEQ ID NO:386), FIG. 388 (SEQ ID NO:388), FIG. 390 (SEQ ID NO:390), FIG. 392 (SEQ ID NO:392), FIG. 394 (SEQ ID NO:394), FIG. 396 (SEQ ID NO:396), FIG. 398 (SEQ ID NO:398), FIG. 400 (SEQ ID NO:400), FIG. 402 (SEQ ID NO:402), FIG. 404 (SEQ ID NO:404), FIG. 406 (SEQ ID NO:406), FIG. 408 (SEQ ID NO:408), FIG. 410 (SEQ ID NO:410), FIG. 412 (SEQ ID NO:412), FIG. 414 (SEQ ID NO:414), FIG. 416 (SEQ ID NO:416), FIG. 418 (SEQ ID NO:418), FIG. 420 (SEQ ID NO:420), FIG. 422 (SEQ ID NO:422), FIG. 424 (SEQ ID NO:424), FIG. 426 (SEQ ID NO:426), FIG. 428 (SEQ ID NO:428), FIG. 430 (SEQ ID NO:430), FIG. 432 (SEQ ID NO:432), FIG. 434 (SEQ ID NO:434), FIG. 436 (SEQ ID NO:436), FIG. 438 (SEQ ID NO:438), FIG. 440 (SEQ ID NO:440), FIG. 442 (SEQ ID NO:442), FIG. 444 (SEQ ID NO:444), FIG. 446 (SEQ ID NO:446), FIG. 448 (SEQ ID NO:448), FIG. 450 (SEQ ID NO:450), FIG. 452 (SEQ ID NO:452), FIG. 454 (SEQ ID NO:454), FIG. 456 (SEQ ID NO:456), FIG. 458 (SEQ ID NO:458), FIG. 460 (SEQ ID NO:460), FIG. 462 (SEQ ID NO:462), FIG. 464 (SEQ ID NO:464), FIG. 466 (SEQ ID NO:466), FIG. 468 (SEQ ID NO:468), FIG. 470 (SEQ ID NO:470), FIG. 472 (SEQ ID NO:472), FIG. 474 (SEQ ID NO:474), FIG. 476 (SEQ ID NO:476), FIG. 478 (SEQ ID NO:478), FIG. 480 (SEQ ID NO:480), FIG. 482 (SEQ ID NO:482), FIG. 484 (SEQ ID NO:484), FIG. 486 (SEQ ID NO:486), FIG. 488 (SEQ ID NO:488), FIG. 490 (SEQ ID NO:490), FIG. 492 (SEQ ID NO:492), FIG. 494 (SEQ ID NO:494), FIG. 496 (SEQ ID NO:496), FIG. 498 (SEQ ID NO:498), FIG. 500 (SEQ ID NO:500), FIG. 502 (SEQ ID NO:502), FIG. 504 (SEQ ID NO:504), FIG. 506 (SEQ ID NO:506), FIG. 508 (SEQ ID NO:508), FIG. 510 (SEQ ID NO:510), FIG. 512 (SEQ ID NO:512), FIG. 514 (SEQ ID NO:514), FIG. 516 (SEQ ID NO:516), FIG. 518 (SEQ ID NO:518), FIG. 520 (SEQ ID NO:520), FIG. 522 (SEQ ID NO:522), FIG. 524 (SEQ ID NO:524), FIG. 526 (SEQ ID NO:526), FIG. 528 (SEQ ID NO:528), FIG. 530 (SEQ ID NO:530), FIG. 532 (SEQ ID NO:532), FIG. 534 (SEQ ID NO:534), FIG. 536 (SEQ ID NO:536), FIG. 538 (SEQ ID NO:538), FIG. 540 (SEQ ID NO:540), FIG. 542 (SEQ ID NO:542), FIG. 544 (SEQ ID NO:544), FIG. 546 (SEQ ID NO:546), FIG. 549 (SEQ ID NO:549), FIG. 551 (SEQ ID NO:551), FIG. 553 (SEQ ID NO:553), FIG. 555 (SEQ ID NO:555), FIG. 557 (SEQ ID NO:557), FIG. 559 (SEQ ID NO:559), FIG. 561 (SEQ ID NO:561), FIG. 563 (SEQ ID NO:563), FIG. 565 (SEQ ID NO:565), FIG. 567 (SEQ ID NO:567), FIG. 569 (SEQ ID NO:569), FIG. 571 (SEQ ID NO:571), FIG. 573 (SEQ ID NO:573), FIG. 575 (SEQ ID NO:575), FIG. 577 (SEQ ID NO:577), FIG. 579 (SEQ ID NO:579), FIG. 581 (SEQ ID NO:581), FIG. 583 (SEQ ID NO:583), FIG. 585 (SEQ ID NO:585), FIG. 587 (SEQ ID NO:587), FIG. 589 (SEQ ID NO:589), FIG. 591 (SEQ ID NO:591), FIG. 593 (SEQ ID NO:593), FIG. 595 (SEQ ID NO:595), FIG. 597 (SEQ ID NO:597), FIG. 599 (SEQ ID NO:599), FIG. 601 (SEQ ID NO:601), FIG. 603 (SEQ ID NO:603), FIG. 605 (SEQ ID NO:605), FIG. 607 (SEQ ID NO:607), FIG. 609 (SEQ ID NO:609), FIG. 611 (SEQ ID NO:611), FIG. 613 (SEQ ID NO:613), FIG. 615 (SEQ ID NO:615), FIG. 617 (SEQ ID NO:617), FIG. 619 (SEQ ID NO:619), FIG. 621 (SEQ ID NO:621), FIG. 623 (SEQ ID NO:623), FIG. 625 (SEQ ID NO:625), FIG. 627 (SEQ ID NO:627), FIG. 629 (SEQ ID NO:629), FIG. 631 (SEQ ID NO:631), FIG. 633 (SEQ ID NO:633), FIG. 635 (SEQ ID NO:635), FIG. 637 (SEQ ID NO:637), FIG. 639 (SEQ ID NO:639), FIG. 641 (SEQ ID NO:641), FIG. 643 (SEQ ID NO:643), FIG. 645 (SEQ ID NO:645), FIG. 647 (SEQ ID NO:647), FIG. 649 (SEQ ID NO:649), FIG. 651 (SEQ ID NO:651), FIG. 653 (SEQ ID NO:653), FIG. 655 (SEQ ID NO:655), FIG. 657 (SEQ ID NO:657), FIG. 659 (SEQ ID NO:659), FIG. 661 (SEQ ID NO:661), FIG. 663 (SEQ ID NO:663), FIG. 665 (SEQ ID NO:665), FIG. 667 (SEQ ID NO:667), FIG. 669 (SEQ ID NO:669), FIG. 671 (SEQ ID NO:671), FIG. 673 (SEQ ID NO:673), FIG. 675 (SEQ ID NO:675), FIG. 677 (SEQ ID NO:377), FIG. 679 (SEQ ID NO:679), FIG. 681 (SEQ ID NO:681), FIG. 683 (SEQ ID NO:683), FIG. 685 (SEQ ID NO:685), FIG. 687 (SEQ ID NO:687), FIG. 689 (SEQ ID NO:689), FIG. 691 (SEQ ID NO:691), FIG. 693 (SEQ ID NO:693), FIG. 695 (SEQ ID NO:695), FIG. 697 (SEQ ID NO:697), FIG. 699 (SEQ ID NO:699), FIG. 701 (SEQ ID NO:701), FIG. 703 (SEQ ID NO:703), FIG. 705 (SEQ ID NO:705), FIG. 707 (SEQ ID NO:707), FIG. 709 (SEQ ID NO:709), FIG. 711 (SEQ ID NO:711), FIG. 713 (SEQ ID NO:713), FIG. 715 (SEQ ID NO:715), FIG. 717 (SEQ ID NO:717), FIG. 719 (SEQ ID NO:719), FIG. 721 (SEQ ID NO:721), FIG. 723 (SEQ ID NO:723), FIG. 725 (SEQ ID NO:725), FIG. 727 (SEQ ID NO:727), FIG. 729 (SEQ ID NO:729), FIG. 731 (SEQ ID NO:731), FIG. 733 (SEQ ID NO:733), FIG. 735 (SEQ ID NO:735), FIG. 737 (SEQ ID NO:737), FIG. 739 (SEQ ID NO:739), FIG. 741 (SEQ ID NO:741), FIG. 743 (SEQ ID NO:743), FIG. 745 (SEQ ID NO:745), FIG. 747 (SEQ ID NO:747), FIG. 749 (SEQ ID NO:749), FIG. 751 (SEQ ID NO:751), FIG. 753 (SEQ ID NO:753), FIG. 755 (SEQ ID NO:755), FIG. 757 (SEQ ID NO:757), FIG. 759 (SEQ ID NO:759), FIG. 761 (SEQ ID NO:761), FIG. 763 (SEQ ID NO:763), FIG. 765 (SEQ ID NO:765), FIG. 767 (SEQ ID NO:767); FIG. 769 (SEQ ID NO:769), FIG. 771 (SEQ ID NO:771), FIG. 773 (SEQ ID NO:773), FIG. 775 (SEQ ID NO:775), FIG. 777 (SEQ ID NO:777), FIG. 779 (SEQ ID NO:779), FIG. 781 (SEQ ID NO:781), FIG. 783 (SEQ ID NO:783), FIG. 785 (SEQ ID NO:785), FIG. 787 (SEQ ID NO:787), FIG. 789 (SEQ ID NO:789), FIG. 791 (SEQ ID NO:791), FIG. 793 (SEQ ID NO:793), FIG. 795 (SEQ ID NO:795), FIG. 797 (SEQ ID NO:797), FIG. 799 (SEQ ID NO:799), FIG. 801 (SEQ ID NO:801), FIG. 803 (SEQ ID NO:803), FIG. 805 (SEQ ID NO:805), FIG. 807 (SEQ ID NO:807), FIG. 809 (SEQ ID NO:809), FIG. 811 (SEQ ID NO:811), FIG. 813 (SEQ ID NO:813), FIG. 815 (SEQ ID NO:815), FIG. 817 (SEQ ID NO:817), FIG. 819 (SEQ ID NO:819), FIG. 821 (SEQ ID NO:821), FIG. 823 (SEQ ID NO:823), FIG. 825 (SEQ ID NO:825), FIG. 827 (SEQ ID NO:827), FIG. 829 (SEQ ID NO:829), FIG. 831 (SEQ ID NO:831), FIG. 833 (SEQ ID NO:833), FIG. 835 (SEQ ID NO:835), FIG. 837 (SEQ ID NO:837), FIG. 839 (SEQ ID NO:839), FIG. 841 (SEQ ID NO:841), FIG. 843 (SEQ ID NO:843), FIG. 845 (SEQ ID NO:845), FIG. 847 (SEQ ID NO:847), FIG. 849 (SEQ ID NO:849), FIG. 851 (SEQ ID NO:851), FIG. 853 (SEQ ID NO:853), FIG. 855 (SEQ ID NO:855), FIG. 858 (SEQ ID NO:858), FIG. 860 (SEQ ID NO:860), FIG. 862 (SEQ ID NO:862), FIG. 864 (SEQ ID NO:864), FIG. 866 (SEQ ID NO:866), FIG. 868 (SEQ ID NO:868), FIG. 870 (SEQ ID NO:870), FIG. 872 (SEQ ID NO:872), FIG. 874 (SEQ ID NO:874), FIG. 876 (SEQ ID NO:876), FIG. 878 (SEQ ID NO:878), FIG. 880 (SEQ ID NO:880), FIG. 882 (SEQ ID NO:882), FIG. 884 (SEQ ID NO:884), FIG. 886 (SEQ ID NO:886), FIG. 888 (SEQ ID NO:888), FIG. 890 (SEQ ID NO:890), FIG. 892 (SEQ ID NO:892), FIG. 894 (SEQ ID NO:894), FIG. 896 (SEQ ID NO:896), FIG. 898 (SEQ ID NO:898), FIG. 900 (SEQ ID NO:900), FIG. 902 (SEQ ID NO:902), FIG. 904 (SEQ ID NO:904), FIG. 906 (SEQ ID NO:906), FIG. 908 (SEQ ID NO:908), FIG. 910 (SEQ ID NO:910), FIG. 912 (SEQ ID NO:912), FIG. 914 (SEQ ID NO:914), FIG. 916 (SEQ ID NO:916), FIG. 918 (SEQ ID NO:918), FIG. 920 (SEQ ID NO:920), FIG. 922 (SEQ ID NO:922), FIG. 924 (SEQ ID NO:924), FIG. 926 (SEQ ID NO:926), FIG. 928 (SEQ ID NO:928), FIG. 930 (SEQ ID NO:930), FIG. 932 (SEQ ID NO:932), FIG. 934 (SEQ ID NO:934), FIG. 936 (SEQ ID NO:936), FIG. 938 (SEQ ID NO:938), FIG. 940 (SEQ ID NO:940), FIG. 943 (SEQ ID NO:943), FIG. 945 (SEQ ID NO:945), FIG. 947 (SEQ ID NO:947), FIG. 949 (SEQ ID NO:949), FIG. 951 (SEQ ID NO:951), FIG. 953 (SEQ ID NO:953), FIG. 955 (SEQ ID NO:955), FIG. 958 (SEQ ID NO:958), FIG. 960 (SEQ ID NO:960), FIG. 963 (SEQ ID NO:963), FIG. 965 (SEQ ID NO:965), FIG. 968 (SEQ ID NO:968), FIG. 970 (SEQ ID NO:970), FIG. 972 (SEQ ID NO:972), FIG. 974 (SEQ ID NO:974), FIG. 976 (SEQ ID NO:976), FIG. 978 (SEQ ID NO:978), FIG. 980 (SEQ ID NO:980), FIG. 983 (SEQ ID NO:983), FIG. 985 (SEQ ID NO:985), FIG. 987 (SEQ ID NO:987), FIG. 990 (SEQ ID NO:990), FIG. 992 (SEQ ID NO:992), FIG. 994 (SEQ ID NO:994), FIG. 996 (SEQ ID NO:996), FIG. 998 (SEQ ID NO:998), FIG. 1000 (SEQ ID NO:1000), FIG. 1002 (SEQ ID NO:1002), FIG. 1007 (SEQ ID NO:1007), FIG. 1009 (SEQ ID NO:1009), FIG. 1011 (SEQ ID NO:1011), FIG. 1013 (SEQ ID NO:1013), FIG. 1015 (SEQ ID NO:1015), FIG. 1017 (SEQ ID NO:1017), FIG. 1019 (SEQ ID NO:1019), FIG. 1021 (SEQ ID NO:1021), FIG. 1023 (SEQ ID NO:1023), FIG. 1025 (SEQ ID NO:1025), FIG. 1030 (SEQ ID NO:1030), FIG. 1033 (SEQ ID NO:1033), FIG. 1035 (SEQ ID NO:1035), FIG. 1037 (SEQ ID NO:1037), FIG. 1039 (SEQ ID NO:1039), FIG. 1041 (SEQ ID NO:1041), FIG. 1043 (SEQ ID NO:1043), FIG. 1046 (SEQ ID NO:1046), FIG. 1048 (SEQ ID NO:1048), FIG. 1050 (SEQ ID NO:1050), FIG. 1053 (SEQ ID NO:1053), FIG. 1055 (SEQ ID NO:1055), FIG. 1057 (SEQ ID NO:1057), FIG. 1060 (SEQ ID NO:1060), FIG. 1062 (SEQ ID NO:1062), FIG. 1064 (SEQ ID NO:1064), FIG. 1066 (SEQ ID NO:1066), FIG. 1068 (SEQ ID NO:1068), FIG. 1070 (SEQ ID NO:1070), FIG. 1072 (SEQ ID NO:1072), FIG. 1074 (SEQ ID NO:1074), FIG. 1076 (SEQ ID NO:1076), FIG. 1078 (SEQ ID NO:1078), FIG. 1080 (SEQ ID NO:1080), FIG. 1082 (SEQ ID NO:1082), FIG. 1084 (SEQ ID NO:1084), FIG. 1086 (SEQ ID NO:1086), FIG. 1088 (SEQ ID NO:1088), FIG. 1090 (SEQ ID NO:1090), FIG. 1092 (SEQ ID NO:1092), FIG. 1094 (SEQ ID NO:1094), FIG. 1096 (SEQ ID NO:1096), FIG. 1098 (SEQ ID NO:1098), FIG. 1101 (SEQ ID NO:1101), FIG. 1103 (SEQ ID NO:1103), FIG. 1105 (SEQ ID NO:1105), FIG. 1107 (SEQ ID NO:1107), FIG. 1109 (SEQ ID NO:1109), FIG. 1111 (SEQ ID NO:1111), FIG. 1113 (SEQ ID NO:1113), FIG. 1116 (SEQ ID NO:1116), FIG. 1118 (SEQ ID NO:1118), FIG. 1120 (SEQ ID NO:1120), FIG. 1123 (SEQ ID NO:1123), FIG. 1125 (SEQ ID NO:1125), FIG. 1128 (SEQ ID NO:1128), FIG. 1130 (SEQ ID NO:1130), FIG. 1132 (SEQ ID NO:1132), FIG. 1134 (SEQ ID NO:1134), FIG. 1136 (SEQ ID NO:1136), FIG. 1138 (SEQ ID NO:1138), FIG. 1142 (SEQ ID NO:1142), FIG. 1144 (SEQ ID NO:1144), FIG. 1146 (SEQ ID NO:1146), FIG. 1148 (SEQ ID NO:1148), FIG. 1150 (SEQ ID NO:1150), FIG. 1152 (SEQ ID NO:1152), FIG. 1154 (SEQ ID NO:1154), FIG. 1156 (SEQ ID NO:1156), FIG. 1158 (SEQ ID NO:1158), FIG. 1160 (SEQ ID NO:1160), FIG. 1162 (SEQ ID NO:1162), FIG. 1164 (SEQ ID NO:1164), FIG. 1166 (SEQ ID NO:1166), FIG. 1168 (SEQ ID NO:1168), FIG. 1170 (SEQ ID NO:1170), FIG. 1172 (SEQ ID NO:1172), FIG. 1174 (SEQ ID NO:1174), FIG. 1176 (SEQ ID NO:1176), FIG. 1178 (SEQ ID NO:1178), FIG. 1180 (SEQ ID NO:1180), FIG. 1182 (SEQ ID NO:1182), FIG. 1184 (SEQ ID NO:1184), FIG. 1186 (SEQ ID NO:1186), FIG. 1188 (SEQ ID NO:1188), FIG. 1190 (SEQ ID NO:1190), FIG. 1192 (SEQ ID NO:1192), FIG. 1194 (SEQ ID NO:1194), FIG. 1196 (SEQ ID NO:1196), FIG. 1198 (SEQ ID NO:1198), FIG. 1200 (SEQ ID NO:1200), FIG. 1202 (SEQ ID NO:1202), FIG. 1204 (SEQ ID NO:1204), FIG. 1206 (SEQ ID NO:1206), FIG. 1208 (SEQ ID NO:1208), FIG. 1210 (SEQ ID NO:1210), FIG. 1212 (SEQ ID NO:1212), FIG. 1214 (SEQ ID NO:1214), FIG. 1216 (SEQ ID NO:1216), FIG. 1218 (SEQ ID NO:1218), FIG. 1220 (SEQ ID NO:1220), FIG. 1222 (SEQ ID NO:1222), FIG. 1224 (SEQ ID NO:1224), FIG. 1226 (SEQ ID NO:1226), FIG. 1228 (SEQ ID NO:1228), FIG. 1230 (SEQ ID NO:1230), FIG. 1232 (SEQ ID NO:1232), FIG. 1234 (SEQ ID NO:1234), FIG. 1236 (SEQ ID NO:1236), FIG. 1238 (SEQ ID NO:1238), FIG. 1240 (SEQ ID NO:1240), FIG. 1242 (SEQ ID NO:1242), FIG. 1244 (SEQ ID NO:1244), FIG. 1246 (SEQ ID NO:1246), FIG. 1248 (SEQ ID NO:1248), FIG. 1250 (SEQ ID NO:1250), FIG. 1252 (SEQ ID NO:1252), FIG. 1254 (SEQ ID NO:1254), FIG. 1256 (SEQ ID NO:1256), FIG. 1258 (SEQ ID NO:1258), FIG. 1260 (SEQ ID NO:1260), FIG. 1262 (SEQ ID NO:1262), FIG. 1264 (SEQ ID NO:1264), FIG. 1266 (SEQ ID NO:1266), FIG. 1268 (SEQ ID NO:1268), FIG. 1270 (SEQ ID NO:1270), FIG. 1272 (SEQ ID NO:1272), FIG. 1274 (SEQ ID NO:1274), FIG. 1276 (SEQ ID NO:1276), FIG. 1278 (SEQ ID NO:1278), FIG. 1280 (SEQ ID NO:1280), FIG. 1282 (SEQ ID NO:1282), FIG. 1284 (SEQ ID NO:1284), FIG. 1286 (SEQ ID NO:1286), FIG. 1288 (SEQ ID NO:1288), FIG. 1290 (SEQ ID NO:1290), FIG. 1292 (SEQ ID NO:1292), FIG. 1294 (SEQ ID NO:1294), FIG. 1296 (SEQ ID NO:1296), FIG. 1298 (SEQ ID NO:1298), FIG. 1300 (SEQ ID NO:1300), FIG. 1302 (SEQ ID NO:1302), FIG. 1304 (SEQ ID NO:1304), FIG. 1306 (SEQ ID NO:1306), FIG. 1308 (SEQ ID NO:1308), FIG. 1310 (SEQ ID NO:1310), FIG. 1312 (SEQ ID NO:1312), FIG. 1314 (SEQ ID NO:1314), FIG. 1316 (SEQ ID NO:1316), FIG. 1318 (SEQ ID NO:1318), FIG. 1320 (SEQ ID NO:1320), FIG. 1322 (SEQ ID NO:1322), FIG. 1324 (SEQ ID NO:1324), FIG. 1326 (SEQ ID NO:1326), FIG. 1328 (SEQ ID NO:1328), FIG. 1330 (SEQ ID NO:1330), FIG. 1332 (SEQ ID NO:1332), FIG. 1334 (SEQ ID NO:1334), FIG. 1336 (SEQ ID NO:1336), FIG. 1338 (SEQ ID NO:1338), FIG. 1340 (SEQ ID NO:1340), FIG. 1342 (SEQ ID NO:1342), FIG. 1344 (SEQ ID NO:1344), FIG. 1346 (SEQ ID NO:1346), FIG. 1348 (SEQ ID NO:1348), FIG. 1350 (SEQ ID NO:1350), FIG. 1352 (SEQ ID NO:1352), FIG. 1354 (SEQ ID NO:1354), FIG. 1356 (SEQ ID NO:1356), FIG. 1358 (SEQ ID NO:1358), FIG. 1361 (SEQ ID NO:1361), FIG. 1363 (SEQ ID NO:1363), FIG. 1365 (SEQ ID NO:1365), FIG. 1367 (SEQ ID NO:1367), FIG. 1369 (SEQ ID NO:1369), FIG. 1371 (SEQ ID NO:1371), FIG. 1373 (SEQ ID NO:1373), FIG. 1375 (SEQ ID NO:1375), FIG. 1377 (SEQ ID NO:1377), FIG. 1379 (SEQ ID NO:1379), FIG. 1381 (SEQ ID NO:1381), FIG. 1383 (SEQ ID NO:1383), FIG. 1385 (SEQ ID NO:1385), FIG. 1387 (SEQ ID NO:1387), FIG. 1389 (SEQ ID NO:1389), FIG. 1391 (SEQ ID NO:1391), FIG. 1393 (SEQ ID NO:1393), FIG. 1395 (SEQ ID NO:1395), FIG. 1397 (SEQ ID NO:1397), FIG. 1399 (SEQ ID NO:1399), FIG. 1401 (SEQ ID NO:1401), FIG. 1403 (SEQ ID NO:1403), FIG. 1405 (SEQ ID NO:1405), FIG. 1407 (SEQ ID NO:1407), FIG. 1409 (SEQ ID NO:1409), FIG. 1411 (SEQ ID NO:1411), FIG. 1413 (SEQ ID NO:1413), FIG. 1415 (SEQ ID NO:1415), FIG. 1417 (SEQ ID NO:1417), FIG. 1419 (SEQ ID NO:1419), FIG. 1421 (SEQ ID NO:1421), FIG. 1423 (SEQ ID NO:1423), FIG. 1425 (SEQ ID NO:1425), FIG. 1427 (SEQ ID NO:1427), FIG. 1429 (SEQ ID NO:1429), FIG. 1431 (SEQ ID NO:1431), FIG. 1433 (SEQ ID NO:1433), FIG. 1435 (SEQ ID NO:1435), FIG. 1437 (SEQ ID NO:1437), FIG. 1439 (SEQ ID NO:1439), FIG. 1441 (SEQ ID NO:1441), FIG. 1443 (SEQ ID NO:1443), FIG. 1445 (SEQ ID NO:1445), FIG. 1447 (SEQ ID NO:1447), FIG. 1449 (SEQ ID NO:1449), FIG. 1451 (SEQ ID NO:1451), FIG. 1453 (SEQ ID NO:1453), FIG. 1455 (SEQ ID NO:1455), FIG. 1457 (SEQ ID NO:1457), FIG. 1459 (SEQ ID NO:1459), FIG. 1461 (SEQ ID NO:1461), FIG. 1463 (SEQ ID NO:1463), FIG. 1465 (SEQ ID NO:1465), FIG. 1467 (SEQ ID NO:1467), FIG. 1469 (SEQ ID NO:1469), FIG. 1471 (SEQ ID NO:1471), FIG. 1473 (SEQ ID NO:1473), FIG. 1475 (SEQ ID NO:1475), FIG. 1477 (SEQ ID NO:1477), FIG. 1479 (SEQ ID NO:1479), FIG. 1481 (SEQ ID NO:1481), FIG. 1483 (SEQ ID NO:1483), FIG. 1485 (SEQ ID NO:1485), FIG. 1487 (SEQ ID NO:1487), FIG. 1489 (SEQ ID NO:1489), FIG. 1491 (SEQ ID NO:1491), FIG. 1493 (SEQ ID NO:1493), FIG. 1495 (SEQ ID NO:1495), FIG. 1497 (SEQ ID NO:1497), FIG. 1499 (SEQ ID NO:1499), FIG. 1501 (SEQ ID NO:1501), FIG. 1503 (SEQ ID NO:1503), FIG. 1505 (SEQ ID NO:1505), FIG. 1507 (SEQ ID NO:1507), FIG. 1509 (SEQ ID NO:1509), FIG. 1511 (SEQ ID NO:1511), FIG. 1513 (SEQ ID NO:1513), FIG. 1515 (SEQ ID NO:1515), FIG. 1517 (SEQ ID NO:1517), FIG. 1519 (SEQ ID NO:1519), FIG. 1521 (SEQ ID NO:1521), FIG. 1523 (SEQ ID NO:1523), FIG. 1525 (SEQ ID NO:1525), FIG. 1527 (SEQ ID NO:1527), FIG. 1529 (SEQ ID NO:1529), FIG. 1531 (SEQ ID NO:1531), FIG. 1533 (SEQ ID NO:1533), FIG. 1535 (SEQ ID NO:1535), FIG. 1537 (SEQ ID NO:1537), FIG. 1539 (SEQ ID NO:1539), FIG. 1541 (SEQ ID NO:1541), FIG. 1543 (SEQ ID NO:1543), FIG. 1545 (SEQ ID NO:1545), FIG. 1547 (SEQ ID NO:1547), FIG. 1549 (SEQ ID NO:1549), FIG. 1551 (SEQ ID NO:1551), FIG. 1553 (SEQ ID NO:1553), FIG. 1555 (SEQ ID NO:1555), FIG. 1557 (SEQ ID NO:1557), FIG. 1559 (SEQ ID NO:1559), FIG. 1561 (SEQ ID NO:1561), FIG. 1563 (SEQ ID NO:1563), FIG. 1565 (SEQ ID NO:1565), FIG. 1567 (SEQ ID NO:1567), FIG. 1569 (SEQ ID NO:1569), FIG. 1571 (SEQ ID NO:1571), FIG. 1573 (SEQ ID NO:1573), FIG. 1575 (SEQ ID NO:1575), FIG. 1578 (SEQ ID NO:1578), FIG. 1581 (SEQ ID NO:1581), FIG. 1583 (SEQ ID NO:1583), FIG. 1585 (SEQ ID NO:1585), FIG. 1587 (SEQ ID NO:1587), FIG. 1589 (SEQ ID NO:1589), FIG. 1592 (SEQ ID NO:1592), FIG. 1594 (SEQ ID NO:1594), FIG. 1596 (SEQ ID NO:1596), FIG. 1598 (SEQ ID NO:1598), FIG. 1600 (SEQ ID NO:1600), FIG. 1602 (SEQ ID NO:1602), FIG. 1604 (SEQ ID NO:1604), FIG. 1606 (SEQ ID NO:1606), FIG. 1609 (SEQ ID NO:1609), FIG. 1611 (SEQ ID NO:1611), FIG. 1614 (SEQ ID NO:1614), FIG. 1617 (SEQ ID NO:1617), FIG. 1619 (SEQ ID NO:1619), FIG. 1621 (SEQ ID NO:1621), FIG. 1623 (SEQ ID NO:1623), FIG. 1626 (SEQ ID NO:1626), FIG. 1628 (SEQ ID NO:1628), FIG. 1630 (SEQ ID NO:1630), FIG. 1632 (SEQ ID NO:1632), FIG. 1636 (SEQ ID NO:1636), FIG. 1639 (SEQ ID NO:1639), FIG. 1644 (SEQ ID NO:1644), FIG. 1646 (SEQ ID NO:1646), FIG. 1648 (SEQ ID NO:1648), FIG. 1650 (SEQ ID NO:1650), FIG. 1652 (SEQ ID NO:1652), FIG. 1654 (SEQ ID NO:1654), FIG. 1656 (SEQ ID NO:1656), FIG. 1658 (SEQ ID NO:1658), FIG. 1660 (SEQ ID NO:1660), FIG. 1662 (SEQ ID NO:1662), FIG. 1664 (SEQ ID NO:1664), FIG. 1666 (SEQ ID NO:1666), FIG. 1668 (SEQ ID NO:1668), FIG. 1670 (SEQ ID NO:1670), FIG. 1672 (SEQ ID NO:1672), FIG. 1674 (SEQ ID NO:1674), FIG. 1676 (SEQ ID NO:1676), FIG. 1678 (SEQ ID NO:1678), FIG. 1680 (SEQ ID NO:1680), FIG. 1683 (SEQ ID NO:1683), FIG. 1685 (SEQ ID NO:1685), FIG. 1687 (SEQ ID NO:1687), FIG. 1689 (SEQ ID NO:1689), FIG. 1692 (SEQ ID NO:1692), FIG. 1694 (SEQ ID NO:1694), FIG. 1696 (SEQ ID NO:1696), FIG. 1698 (SEQ ID NO:1698), FIG. 1701 (SEQ ID NO:1701), FIG. 1703 (SEQ ID NO:1703), FIG. 1705 (SEQ ID NO:1705), FIG. 1707 (SEQ ID NO:1707), FIG. 1709 (SEQ ID NO:1709), FIG. 1711 (SEQ ID NO:1711), FIG. 1713 (SEQ ID NO:1713), FIG. 1715 (SEQ ID NO:1715), FIG. 1717 (SEQ ID NO:1717), FIG. 1719 (SEQ ID NO:1719), FIG. 1721 (SEQ ID NO:1721), FIG. 1723 (SEQ ID NO:1723), FIG. 1725 (SEQ ID NO:1725), FIG. 1727 (SEQ ID NO:1727), FIG. 1729 (SEQ ID NO:1729), FIG. 1732 (SEQ ID NO:1732), FIG. 1734 (SEQ ID NO:1734), FIG. 1736 (SEQ ID NO:1736), FIG. 1738 (SEQ ID NO:1738), FIG. 1741 (SEQ ID NO:1741), FIG. 1743 (SEQ ID NO:1743), FIG. 1745 (SEQ ID NO:1745), FIG. 1747 (SEQ ID NO:1747), FIG. 1749 (SEQ ID NO:1749), FIG. 1751 (SEQ ID NO:1751), FIG. 1753 (SEQ ID NO:1753), FIG. 1755 (SEQ ID NO:1755), FIG. 1757 (SEQ ID NO:1757), FIG. 1760 (SEQ ID NO:1760), FIG. 1762 (SEQ ID NO:1762), FIG. 1764 (SEQ ID NO:1764), FIG. 1766 (SEQ ID NO:1766), FIG. 1769 (SEQ ID NO:1769), FIG. 1771 (SEQ ID NO:1771), FIG. 1773 (SEQ ID NO:1773), FIG. 1775 (SEQ ID NO:1775), FIG. 1777 (SEQ ID NO:1777), FIG. 1779 (SEQ ID NO:1779), FIG. 1781 (SEQ ID NO:1781), FIG. 1783 (SEQ ID NO:1783), FIG. 1785 (SEQ ID NO:1785), FIG. 1787 (SEQ ID NO:1787), FIG. 1789 (SEQ ID NO:1789), FIG. 1792 (SEQ ID NO:1792), FIG. 1795 (SEQ ID NO:1795), FIG. 1797 (SEQ ID NO:1797), FIG. 1799 (SEQ ID NO:1799), FIG. 1802 (SEQ ID NO:1802), FIG. 1805 (SEQ ID NO:1805), FIG. 1807 (SEQ ID NO:1807), FIG. 1809 (SEQ ID NO:1809), FIG. 1811 (SEQ ID NO:1811), FIG. 1813 (SEQ ID NO:1813), FIG. 1815 (SEQ ID NO:1815), FIG. 1817 (SEQ ID NO:1817), FIG. 1819 (SEQ ID NO:1819), FIG. 1821 (SEQ ID NO:1821), FIG. 1823 (SEQ ID NO:1823), FIG. 1825 (SEQ ID NO:1825), FIG. 1827 (SEQ ID NO:1827), FIG. 1829 (SEQ ID NO:1829), FIG. 1831 (SEQ ID NO:1831), FIG. 1833 (SEQ ID NO:1833), FIG. 1836 (SEQ ID NO:1836), FIG. 1838 (SEQ ID NO:1838), FIG. 1841 (SEQ ID NO:1841), FIG. 1843 (SEQ ID NO:1843), FIG. 1845 (SEQ ID NO:1845), FIG. 1847 (SEQ ID NO:1847), FIG. 1849 (SEQ ID NO:1849), FIG. 1851 (SEQ ID NO:1851), FIG. 1854 (SEQ ID NO:1854), FIG. 1856 (SEQ ID NO:1856), FIG. 1858 (SEQ ID NO:1858), FIG. 1860 (SEQ ID NO:1860), FIG. 1863 (SEQ ID NO:1863), FIG. 1865 (SEQ ID NO:1865), FIG. 1867 (SEQ ID NO:1867), FIG. 1869 (SEQ ID NO:1869), FIG. 1871 (SEQ ID NO:1871), FIG. 1873 (SEQ ID NO:1873), FIG. 1875 (SEQ ID NO:1875), FIG. 1877 (SEQ ID NO:1877), FIG. 1881 (SEQ ID NO:1881), FIG. 1883 (SEQ ID NO:1883), FIG. 1885 (SEQ ID NO:1885), FIG. 1887 (SEQ ID NO:1887), FIG. 1889 (SEQ ID NO:1889), FIG. 1891 (SEQ ID NO:1891), FIG. 1893 (SEQ ID NO:1893), FIG. 1895 (SEQ ID NO:1895), FIG. 1897 (SEQ ID NO:1897), FIG. 1899 (SEQ ID NO:1899), FIG. 1901 (SEQ ID NO:1901), FIG. 1903 (SEQ ID NO:1903), FIG. 1905 (SEQ ID NO:1905), FIG. 1908 (SEQ ID NO:1908), FIG. 1910 (SEQ ID NO:1910), FIG. 1913 (SEQ ID NO:1913), FIG. 1915 (SEQ ID NO:1915), FIG. 1917 (SEQ ID NO:1917), FIG. 1919 (SEQ ID NO:1919), FIG. 1922 (SEQ ID NO:1922), FIG. 1924 (SEQ ID NO:1924), FIG. 1926 (SEQ ID NO:1926), FIG. 1928 (SEQ ID NO:1928), FIG. 1930 (SEQ ID NO:1930), FIG. 1932 (SEQ ID NO:1932), FIG. 1934 (SEQ ID NO:1934), FIG. 1936 (SEQ ID NO:1936), FIG. 1938 (SEQ ID NO:1938), FIG. 1940 (SEQ ID NO:1940), FIG. 1942 (SEQ ID NO:1942), FIG. 1944 (SEQ ID NO:1944), FIG. 1946 (SEQ ID NO:1946), FIG. 1948 (SEQ ID NO:1948), FIG. 1950 (SEQ ID NO:1950), FIG. 1952 (SEQ ID NO:1952), FIG. 1954 (SEQ ID NO:1954), FIG. 1956 (SEQ ID NO:1956), FIG. 1958 (SEQ ID NO:1958), FIG. 1960 (SEQ ID NO:1960), FIG. 1962 (SEQ ID NO:1962), FIG. 1964 (SEQ ID NO:1964), FIG. 1966 (SEQ ID NO:1966), FIG. 1968 (SEQ ID NO:1968), FIG. 1970 (SEQ ID NO:1970), FIG. 1972 (SEQ ID NO:1972), FIG. 1974 (SEQ ID NO:1974), FIG. 1976 (SEQ ID NO:1976), FIG. 1978 (SEQ ID NO:1978), FIG. 1980 (SEQ ID NO:1980), FIG. 1982 (SEQ ID NO:1982), FIG. 1984 (SEQ ID NO:1984), FIG. 1986 (SEQ ID NO:1986), FIG. 1988 (SEQ ID NO:1988), FIG. 1990 (SEQ ID NO:1990), FIG. 1992 (SEQ ID NO:1992), FIG. 1994 (SEQ ID NO:1994), FIG. 1996 (SEQ ID NO:1996), FIG. 1998 (SEQ ID NO:1998), FIG. 2000 (SEQ ID NO:2000), FIG. 2002 (SEQ ID NO:2002), FIG. 2004 (SEQ ID NO:2004), FIG. 2006 (SEQ ID NO:2006), FIG. 2008 (SEQ ID NO:2008), FIG. 2010 (SEQ ID NO:2010), FIG. 2012 (SEQ ID NO:2012), FIG. 2014 (SEQ ID NO:2014), FIG. 2016 (SEQ ID NO:2016), FIG. 2018 (SEQ ID NO:2018), FIG. 2020 (SEQ ID NO:2020), FIG. 2022 (SEQ ID NO:2022), FIG. 2024 (SEQ ID NO:2024), FIG. 2026 (SEQ ID NO:2026), FIG. 2028 (SEQ ID NO:2028), FIG. 2030 (SEQ ID NO:2030), FIG. 2032 (SEQ ID NO:2032), FIG. 2034 (SEQ ID NO:2034), FIG. 2036 (SEQ ID NO:2036), FIG. 2038 (SEQ ID NO:2038), FIG. 2040 (SEQ ID NO:2040), FIG. 2042 (SEQ ID NO:2042), FIG. 2044 (SEQ ID NO:2044), FIG. 2046 (SEQ ID NO:2046), FIG. 2048 (SEQ ID NO:2048), FIG. 2050 (SEQ ID NO:2050), FIG. 2052 (SEQ ID NO:2052), FIG. 2054 (SEQ ID NO:2054), FIG. 2056 (SEQ ID NO:2056), FIG. 2058 (SEQ ID NO:2058), FIG. 2060 (SEQ ID NO:2060), FIG. 2062 (SEQ ID NO:2062), FIG. 2064 (SEQ ID NO:2064), FIG. 2066 (SEQ ID NO:2066), FIG. 2068 (SEQ ID NO:2068), FIG. 2070 (SEQ ID NO:2070), FIG. 2072 (SEQ ID NO:2072), FIG. 2074 (SEQ ID NO:2074), FIG. 2076 (SEQ ID NO:2076), FIG. 2078 (SEQ ID NO:2078), FIG. 2080 (SEQ ID NO:2080), FIG. 2082 (SEQ ID NO:2082), FIG. 2084 (SEQ ID NO:2084), FIG. 2086 (SEQ ID NO:2086), FIG. 2088 (SEQ ID NO:2088), FIG. 2090 (SEQ ID NO:2090), FIG. 2092 (SEQ ID NO:2092), FIG. 2094 (SEQ ID NO:2094), FIG. 2096 (SEQ ID NO:2096), FIG. 2098 (SEQ ID NO:2098), FIG. 2100 (SEQ ID NO:2100), FIG. 2102 (SEQ ID NO:2102), FIG. 2104 (SEQ ID NO:2104), FIG. 2106 (SEQ ID NO:2106), FIG. 2108 (SEQ ID NO:2108), FIG. 2110 (SEQ ID NO:2110), FIG. 2112 (SEQ ID NO:2112), FIG. 2114 (SEQ ID NO:2114), FIG. 2116 (SEQ ID NO:2116), FIG. 2118 (SEQ ID NO:2118), FIG. 2120 (SEQ ID NO:2120), FIG. 2122 (SEQ ID NO:2122), FIG. 2124 (SEQ ID NO:2124), FIG. 2126 (SEQ ID NO:2126), FIG. 2128 (SEQ ID NO:2128), FIG. 2130 (SEQ ID NO:2130), FIG. 2132 (SEQ ID NO:2132), FIG. 2134 (SEQ ID NO:2134), FIG. 2136 (SEQ ID NO:2136), FIG. 2138 (SEQ ID NO:2138), FIG. 2140 (SEQ ID NO:2140), FIG. 2142 (SEQ ID NO:2142), FIG. 2144 (SEQ ID NO:2144), FIG. 2146 (SEQ ID NO:2146), FIG. 2148 (SEQ ID NO:2148), FIG. 2150 (SEQ ID NO:2150), FIG. 2152 (SEQ ID NO:2152), FIG. 2154 (SEQ ID NO:2154), FIG. 2156 (SEQ ID NO:2156), FIG. 2158 (SEQ ID NO:2158), FIG. 2160 (SEQ ID NO:2160), FIG. 2163 (SEQ ID NO:2163), FIG. 2165 (SEQ ID NO:2165), FIG. 2167 (SEQ ID NO:2167), FIG. 2170 (SEQ ID NO:2170), FIG. 2172 (SEQ ID NO:2172), FIG. 2174 (SEQ ID NO:2174), FIG. 2176 (SEQ ID NO:2176), FIG. 2178 (SEQ ID NO:2178), FIG. 2180 (SEQ ID NO:2180), FIG. 2182 (SEQ ID NO:2182), FIG. 2184 (SEQ ID NO:2184), FIG. 2186 (SEQ ID NO:2186), FIG. 2188 (SEQ ID NO:2188), FIG. 2191 (SEQ ID NO:2191), FIG. 2193 (SEQ ID NO:2193), FIG. 2195 (SEQ ID NO:2195), FIG. 2197 (SEQ ID NO:2197), FIG. 2199 (SEQ ID NO:2199), FIG. 2201 (SEQ ID NO:2201), FIG. 2204 (SEQ ID NO:2204), FIG. 2207 (SEQ ID NO:2207), FIG. 2209 (SEQ ID NO:2209), FIG. 2212 (SEQ ID NO:2212), FIG. 2214 (SEQ ID NO:2214), FIG. 2216 (SEQ ID NO:2216), FIG. 2218 (SEQ ID NO:2218), FIG. 2220 (SEQ ID NO:2220), FIG. 2222 (SEQ ID NO:2222), FIG. 2224 (SEQ ID NO:2224), FIG. 2226 (SEQ ID NO:2226), FIG. 2228 (SEQ ID NO:2228), FIG. 2230 (SEQ ID NO:2230), FIG. 2232 (SEQ ID NO:2232), FIG. 2234 (SEQ ID NO:2234), FIG. 2236 (SEQ ID NO:2236), FIG. 2238 (SEQ ID NO:2238), FIG. 2240 (SEQ ID NO:2240), FIG. 2242 (SEQ ID NO:2242), FIG. 2244 (SEQ ID NO:2244), FIG. 2246 (SEQ ID NO:2246), FIG. 2248 (SEQ ID NO:2248), FIG. 2250 (SEQ ID NO:2250), FIG. 2252 (SEQ ID NO:2252), FIG. 2254 (SEQ ID NO:2254), FIG. 2256 (SEQ ID NO:2256), FIG. 2258 (SEQ ID NO:2258), FIG. 2260 (SEQ ID NO:2260), FIG. 2262 (SEQ ID NO:2262), FIG. 2264 (SEQ ID NO:2264), FIG. 2266 (SEQ ID NO:2266), FIG. 2268 (SEQ ID NO:2268), FIG. 2270 (SEQ ID NO:2270), FIG. 2272 (SEQ ID NO:2272), FIG. 2274 (SEQ ID NO:2274), FIG. 2276 (SEQ ID NO:2276), FIG. 2278 (SEQ ID NO:2278), FIG. 2280 (SEQ ID NO:2280), FIG. 2282 (SEQ ID NO:2282), FIG. 2284 (SEQ ID NO:2284), FIG. 2286 (SEQ ID NO:2286), FIG. 2288 (SEQ ID NO:2288), FIG. 2290 (SEQ ID NO:2290), FIG. 2292 (SEQ ID NO:2292), FIG. 2294 (SEQ ID NO:2294), FIG. 2296 (SEQ ID NO:2296), FIG. 2298 (SEQ ID NO:2298), FIG. 2301 (SEQ ID NO:2301), FIG. 2303 (SEQ ID NO:2303), FIG. 2305 (SEQ ID NO:2305), FIG. 2307 (SEQ ID NO:2307), FIG. 2310 (SEQ ID NO:2310), FIG. 2312 (SEQ ID NO:2312), FIG. 2314 (SEQ ID NO:2314), FIG. 2316 (SEQ ID NO:2316), FIG. 2319 (SEQ ID NO:2319), FIG. 2321 (SEQ ID NO:2321), FIG. 2323 (SEQ ID NO:2323), FIG. 2325 (SEQ ID NO:2325), FIG. 2327 (SEQ ID NO:2327), FIG. 2329 (SEQ ID NO:2329), FIG. 2331 (SEQ ID NO:2331), FIG. 2333 (SEQ ID NO:2333), FIG. 2335 (SEQ ID NO:2335), FIG. 2337 (SEQ ID NO:2337), FIG. 2339 (SEQ ID NO:2339), FIG. 2341 (SEQ ID NO:2341), FIG. 2343 (SEQ ID NO:2343), FIG. 2345 (SEQ ID NO:2345), FIG. 2347 (SEQ ID NO:2347), FIG. 2349 (SEQ ID NO:2349), FIG. 2351 (SEQ ID NO:2351), FIG. 2353 (SEQ ID NO:2353), FIG. 2355 (SEQ ID NO:2355), FIG. 2357 (SEQ ID NO:2357), FIG. 2359 (SEQ ID NO:2359), FIG. 2361 (SEQ ID NO:2361), FIG. 2363 (SEQ ID NO:2363), FIG. 2365 (SEQ ID NO:2365), FIG. 2367 (SEQ ID NO:2367), FIG. 2369 (SEQ ID NO:2369), FIG. 2371 (SEQ ID NO:2371), FIG. 2373 (SEQ ID NO:2373), FIG. 2375 (SEQ ID NO:2375), FIG. 2378 (SEQ ID NO:2378), FIG. 2380 (SEQ ID NO:2380), FIG. 2383 (SEQ ID NO:2383), FIG. 2386 (SEQ ID NO:2386), FIG. 2388 (SEQ ID NO:2388), FIG. 2391 (SEQ ID NO:2391), FIG. 2393 (SEQ ID NO:2393), FIG. 2396 (SEQ ID NO:2396), FIG. 2398 (SEQ ID NO:2398), FIG. 2402 (SEQ ID NO:2402), FIG. 2404 (SEQ ID NO:2404), FIG. 2408 (SEQ ID NO:2408), FIG. 2410 (SEQ ID NO:2410), FIG. 2412 (SEQ ID NO:2412), FIG. 2415 (SEQ ID NO:2415), FIG. 2418 (SEQ ID NO:2418), FIG. 2420 (SEQ ID NO:2420), FIG. 2422 (SEQ ID NO:2422), FIG. 2424 (SEQ ID NO:2424), FIG. 2426 (SEQ ID NO:2426), FIG. 2430 (SEQ ID NO:2430), FIG. 2432 (SEQ ID NO:2432), FIG. 2434 (SEQ ID NO:2434), FIG. 2436 (SEQ ID NO:2436), FIG. 2438 (SEQ ID NO:2438), FIG. 2440 (SEQ ID NO:2440), or FIG. 2442 (SEQ ID NO:2442) (a) in a test sample of tissue cells obtained from the mammal, and (b) in a control sample of known normal tissue cells of the same cell type, wherein a higher or lower level of expression of said gene in the test sample as compared to the control sample is indicative of the presence of an inflammatory immune response in the mammal from which the test tissue cells were obtained.
